(19) United States
(12) Patent Application Publication
(10) Pub. No.: US 2024/0206311 A1
(43) Pub. Date: Jun. 20, 2024

(54) ORGANIC ELECTROLUMINESCENT DEVICE

(71) Applicant: ROHM AND HAAS ELECTRONIC MATERIALS KOREA LTD., Chungcheongnam-do (KR)

(72) Inventors: Eun-Joung CHOI, Gyeonggi-do (KR); Hae-Yeon KIM, Gyeonggi-do (KR); Jin-Man KIM, Gyeonggi-do (KR); Chi-Sik KIM, Gyeonggi-do (KR); Soo-Yong LEE, Gyeonggi-do (KR); So-Mi PARK, Gyeonggi-do (KR); Jeong-Hwan JEON, Gyeonggi-do (KR); Dong-Hyung LEE, Gyeonggi-do (KR); Dominea RATHWELL, Gyeonggi-do (KR); Yoo-Jin DOH, Gyeonggi-do (KR); Mi-Ran SEO, Gyeonggi-do (KR)

(21) Appl. No.: 18/488,148

(22) Filed: Oct. 17, 2023

(30) Foreign Application Priority Data
Oct. 20, 2022 (KR) 10-2022-0135448
Oct. 16, 2023 (KR) 10-2023-0138090

(51) Int. Cl. H10K 85/20; C09K 11/06; H10K 85/10; H10K 85/40; H10K 85/60

(52) U.S. Cl. CPC H10K 85/20; C09K 11/06; H10K 85/113; H10K 85/40; H10K 85/622; H10K 85/633; H10K 85/654; H10K 85/6572; H10K 85/6574; H10K 85/6576; C07B 2200/05

(57) ABSTRACT

The present disclosure relates to organic electroluminescent devices. The organic electroluminescent device according to the present disclosure exhibits improved lifespan characteristics and progressive driving voltage by including at least one deuterated compound(s).

### TECHNICAL FIELD

The present disclosure relates to an organic electroluminescent device.

### BACKGROUND ART

An organic electroluminescent device (OLED) is a self-light-emitting display device which has advantages in that it provides a wider viewing angle, a greater contrast ratio, and a faster response time. In such an OLED, holes from the anode and electrons from the cathode are injected into a light-emitting layer by the application of electric voltage, and excitons having high energy are produced by the recombination of the holes and electrons. The organic light-emitting compound moves into an excited state by the energy and emits light from energy when the organic light-emitting compound returns to the ground state from the excited state.

In recent years, due to the potential of flat panel displays and general lighting devices, the development of new materials for this is continuously required. The development of excellent high-performance materials and more desirable device structures is required in order to improve the performance required in medium and large-sized OLED panels.

Unlike red and green high-efficiency phosphorescent materials which have already been commercialized among the light-emitting materials of the OLED, it has been pointed out that a blue phosphorescent material is not suitable for long-term use such as several years or more, since the blue phosphorescent material has short lifespan and high driving voltage, and thus, a fluorescent material is used. As such, conventional materials have not been able to satisfy the light-emitting characteristics of the OLED, and thus development of an OLED including an organic electroluminescent material having excellent performance is required.

Korean Patent Application Laid-open No. 2020-0037654 A discloses an OLED in that a phenanthroline derivative is comprised in a charge generation layer material. However, said reference does not specifically disclose an OLED including a combination of a deuterated organic electroluminescent materials specified herein.

### DISCLOSURE OF THE INVENTION

### Technical Problem

The object of the present disclosure is to provide an organic electroluminescent device having improved progressive driving voltage characteristics and long lifespan characteristics.

### Solution to Problem

As a result of intensive studies to solve the technical problem above, the present inventors found that the aforementioned objective can be achieved by an organic electroluminescent device comprising a plurality of light-emitting units positioned between the first electrode and the second electrode; and at least one charge generation layer positioned between the adjacent light-emitting units, wherein the light-emitting units comprise at least one light-emitting layer, and at least one of the light-emitting layer and the charge generation layer comprises a deuterated compound, so that the present invention was completed.

### Advantageous Effects of Invention

The organic electroluminescent device according to the present disclosure exhibits improved lifespan and progressive driving voltage characteristics by comprising a deuterated compound.

### BRIEF DESCRIPTION OF THE DRAWINGS

FIG. 1 shows an example of an organic electroluminescent device according to one embodiment of the present disclosure.

FIG. 2 shows an example of an organic electroluminescent device according to another embodiment of the present disclosure.

### MODE FOR THE INVENTION

Hereinafter, the present disclosure will be described in detail. However, the following description is intended to explain the invention, and is not meant in any way to restrict the scope of the invention.

The present disclosure relates to an organic electroluminescent device comprising a plurality of light-emitting units positioned between the first electrode and the second electrode; and at least one charge generation layer positioned between the adjacent light-emitting units.

In the organic electroluminescent device according to the present disclosure, the light-emitting units comprise at least one light-emitting layer and at least one of the light-emitting layer and the charge generation layer comprises a deuterated compound.

The term “organic electroluminescent compound” in the present disclosure means a compound that may be used in an organic electroluminescent device, and may be comprised in any material layer constituting an organic electroluminescent device, as necessary.

Herein, the term “organic electroluminescent material” means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron blocking material, a light-emitting material (containing host and dopant materials), an electron buffer material, a hole blocking material, an electron transport material, or an electron injection material, etc.

The term “deuterated” in the present disclosure means that one or more hydrogen atoms of a compound or functional group are replaced with deuterium, and includes replacement of some or all of the hydrogen atoms with deuterium.

The term “(C1-C30)alkyl(ene)” in the present disclosure is meant to be a linear or branched alkyl having 1 to 30 carbon atoms constituting the chain, in which the number of carbon atoms is preferably 1 to 20, and more preferably 1 to 10. The above alkyl may include methyl, ethyl, n-propyl, isopropyl, n-butyl, isobutyl, tert-butyl, sec-butyl, etc. The term “(C3-C30)cycloalkyl(ene)” in the present disclosure is meant to be a mono- or polycyclic hydrocarbon having 3 to 30 ring backbone carbon atoms, in which the number of carbon atoms is preferably 3 to 20, and more preferably 3 to 7. The above cycloalkyl may include cyclopropyl, cyclobutyl, cyclopentyl, cyclohexyl, cyclopentylmethyl, cyclohexylmethyl, etc. The term “(C6-C30)aryl(ene)” in the present disclosure is a monocyclic or fused ring radical derived from an aromatic hydrocarbon having 6 to 30 ring backbone carbon atoms, in which the number of the ring backbone carbon atoms is preferably 6 to 20, more preferably 6 to 15, and may be partially saturated. The aryl may comprise a spiro structure. Examples of the aryl specifically include phenyl, biphenyl, terphenyl, quaterphenyl, naphthyl, binaphthyl, phenyinaphthyl, naphthylphenyl, fluorenyl, phenylfluorenyl, dimethyfluorenyl, diphenylfluorenyl, benzofluorenyl, diphenylbenzofluorenyl, dibenzofluorenyl, phenanthrenyl, benzophenanthrenyl, phenylphenanthrenyl, anthracenyl, benzanthracenyl, indenyl, triphenylenyl, pyrenyl, tetracenyl, perylenyl, chrysenyl, benzochrysenyl, naphthacenyl, fluoranthenyl, benzofluoranthenyl, tolyl, xylyl, mesityl, cumenyl, spiro[fluorene-fluorene]yl, spiro[fluorene-benzofluorene]yl, azulenyl, tetramethyl-dihydrophenanthrenyl, etc. More specifically, the aryl may be o-tolyl, m-tolyl, p-tolyl, 2,3-xylyl, 3,4-xylyl, 2,5-xylyl, mesityl, o-cumenyl, m-cumenyl, p-cumenyl, p-t-butylphenyl, p-(2-phenylpropyl)phenyl, 4′-methylbiphenyl, 4″-t-butyl-p-terphenyl-4-yl, o-biphenyl, m-biphenyl, p-biphenyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 1-naphthyl, 2-naphthyl, 1-fluorenyl, 2-fluorenyl, 3-fluorenyl, 4-fluorenyl, 9-fluorenyl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 1-anthryl, 2-anthryl, 9-anthryl, 1-phenanthryl, 2-phenanthryl, 3-phenanthryl, 4-phenanthryl, 9-phenanthryl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 3-fluoranthenyl, 4-fluoranthenyl, 8-fluoranthenyl, 9-fluoranthenyl, benzofluoranthenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, 9,9,10,10-tetramethyl-9,10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc. The term “(3- to 30-membered)heteroaryl(ene)” in the present disclosure is an aryl having 3 to 30 ring backbone atoms including at least one, preferably 1 to 4 heteroatoms selected from the group consisting of B, N, O, S, Si, P, Se, and Ge, in which the number of ring backbone atoms is preferably 3 to 30, more preferably 5 to 20. The above heteroaryl or heteroarylene may be a monocyclic ring, or a fused ring condensed with at least one benzene ring; and may be partially saturated. Also, the above heteroaryl or heteroarylene in the present disclosure may be one formed by linking at least one heteroaryl or aryl group to a heteroaryl group via a single bond(s) and may comprise a spiro structure. Examples of the heteroaryl specifically may include a monocyclic ring-type heteroaryl including furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl including benzofuranyl, benzothiophenyl, isobenzofuranyl, dibenzofuranyl, dibenzothiophenyl, dibenzoselenophenyl, benzofuroquinolinyl, benzofuroquinazolinyl, benzofuronaphthiridinyl, benzofuropyrimidinyl, naphthofuropyrimidinyl, benzothienoquinolinyl, benzothienoquinazolinyl, benzothienonaphthiridinyl, benzothienopyrimidinyl, naphthothienopyrimidinyl, pyrimidoindolyl, benzopyrimidoindolyl, benzofuropyrazinyl, naphthofuropyrazinyl, benzothienopyrazinyl, naphthothienopyrazinyl, pyrazinoindolyl, benzopyrazinoindolyl, benzoimidazolyl, benzothiazolyl, benzoisothiazolyl, benzoisoxazolyl, benzoxazolyl, imidazopyridinyl, isoindolyl, indolyl, benzoindolyl, indazolyl, benzothiadiazolyl, quinolyl, isoquinolyl, cinnolinyl, quinazolinyl, quinoxalinyl, carbazolyl, azacarbazolyl, benzocarbazolyl, dibenzocarbazolyl, phenoxazinyl, phenanthridinyl, benzodioxolyl, indolizidinyl, acridinyl, silafluorenyl, germafluorenyl, benzotriazolyl, phenazinyl, imidazopyridinyl, chromenoquinazolinyl, thiochromenoquinazolinyl, dimethylbenzopyrimidinyl, indolocarbazolyl, indenocarbazolyl, etc. More specifically, the heteroaryl may be 1-pyrrolyl, 2-pyrrolyl, 3-pyrrolyl, 2-pyridinyl, 3-pyridinyl, 4-pyridinyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolizidinyl, 2-indolizidinyl, 3-indolizidinyl, 5-indolizidinyl, 6-indolizidinyl, 7-indolizidinyl, 8-indolizidinyl, 2-imidazopyridinyl, 3-imidazopyridinyl, 5-imidazopyridinyl, 6-imidazopyridinyl, 7-imidazopyridinyl, 8-imidazopyridinyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazol-1-yl, azacarbazol-2-yl, azacarbazol-3-yl, azacarbazol-4-yl, azacarbazol-5-yl, azacarbazol-6-yl, azacarbazol-7-yl, azacarbazol-8-yl, azacarbazol-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-t-butylpyrrol-4-yl, 3-(2-phenylpropyl)pyrrol-1-yl, 2-methyl-1-indolyl, 4-methyl-1-indolyl, 2-methyl-3-indolyl, 4-methyl-3-indolyl, 2-t-butyl-1-indolyl, 4-t-butyl-1-indolyl, 2-t-butyl-3-indolyl, 4-t-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtho-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2,3-b]-benzofuranyl, 10-naphtho-[2,3-b]-benzofuranyl, 1-naphtho-[2,1-b]-benzofuranyl, 2-naphtho-[2,1-b]-benzofuranyl, 3-naphtho-[2,1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2,1-b]-benzothiophenyl, 3-naphtho-[2,1-b]-benzothiophenyl, 4-naphtho-[2,1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 2-benzofuro[3,2-d]pyrimidinyl, 6-benzofuro[3,2-d]pyrmidinyl, 7-benzofuro[3,2-d]pyrmidinyl, 8-benzofuro[3,2-d]pyrimidinyl, 9-benzofuro[3,2-d]pyrimidinyl, 2-benzothio[3,2-d]pyrimidinyl, 6-benzothio[3,2-d]pyrimidinyl, 7-benzothio[3,2-d]pyrimidinyl, 8-benzothio[3,2-d]pyrmidinyl, 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-benzothio[3,2-d]pyrazinyl, 6-benzothio[3,2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc. The term “a fused ring of (C3-C30) aliphatic ring and (C6-C30) aromatic ring” in the present disclosure means a ring formed by fusing at least one aliphatic ring having 3 to 30 ring backbone carbon atoms in which the number of the carbon atoms is preferably 3 to 25, more preferably 3 to 18, and at least one aromatic ring having 6 to 30 ring backbone carbon atoms in which the number of the carbon atoms is preferably 6 to 25, more preferably 6 to 18. For example, the fused ring may be a fused ring of at least one benzene and at least one cyclohexane, or a fused ring of at least one naphthalene and at least one cyclopentane, etc. The carbon atoms in the fused ring of (C3-C30) aliphatic ring and (C6-C30) aromatic ring of the present disclosure may be replaced with at least one heteroatoms selected from B, N, O, S, Si, and P, preferably at least one heteroatoms selected from N, O and S. The term “Halogen” in the present disclosure includes F, Cl, Br, and I.

In addition. “ortho (o),” “meta (m),” and “para (p)” in the present disclosure are meant to signify the substitution position of all substituents. Ortho position is a compound with substituents, which are adjacent to each other, i.e., at the 1 and 2 positions on benzene. Meta position is the next substitution position of the immediately adjacent substitution position, i.e., a compound with substituents at the 1 and 3 positions on benzene. Para position is the next substitution position of the meta position, i.e., a compound with substituents at the 1 and 4 positions on benzene.

The term “a ring formed in linking to an adjacent substituent” in the present disclosure means a substituted or unsubstituted (3- to 50-membered) mono- or polycyclic, alicyclic, aromatic ring, or a combination thereof, formed by linking or fusing two or more adjacent substituents, preferably, may be a substituted or unsubstituted (5- to 40-membered) mono- or polycyclic, alicyclic, aromatic ring, or a combination thereof. Further, the formed ring may include at least one heteroatom selected from the group consisting of B, N, O, S, Si, and P, preferably, at least one heteroatom selected from N, O, and S. According to one embodiment of the present disclosure, the number of atoms in the ring skeleton is 5 to 35; according to another embodiment of the present disclosure, the number of atoms in the ring skeleton is 5 to 30. In one embodiment, the fused ring may be, for example, a substituted or unsubstituted fluorene ring, a substituted or unsubstituted dibenzothiophene ring, a substituted or unsubstituted dibenzofuran ring, a substituted or unsubstituted naphthalene ring, a substituted or unsubstituted phenanthrene ring, a substituted or unsubstituted benzofluorene ring, a substituted or unsubstituted benzothiophene ring, a substituted or unsubstituted benzofuran ring, a substituted or unsubstituted indole ring, a substituted or unsubstituted indene ring, a substituted or unsubstituted benzene ring, or a substituted or unsubstituted carbazole ring, etc.

In addition, ‘substituted’ in the expression ‘substituted or unsubstituted’ means that a hydrogen atom in a certain functional group is replaced with another atom or functional group, i.e., a substituent, and substituted with a group to which two or more substituents are connected among the substituents. For example, “a substituent to which two or more substituents are connected” may be pyridine-triazine. That is, pyridine-triazine may be heteroaryl or may be interpreted as a substituent in which two heteroaryls are connected. Preferably, the substituted alkyl, the substituted alkenyl, the substituted aryl(ene), the substituted heteroaryl(ene), the substituted cycloalkyl, the substituted cycloalkenyl, the substituted heterocycloalkyl, the substituted alkoxy, the substituted trialkylsilyl, the substituted dialkylarylsilyl, the substituted alkyldiarylsilyl, the substituted triarylsilyl, the substituted fused ring of aliphatic ring and aromatic ring, the substituted mono- or di-alkylamino, the substituted mono- or di-alkenylamino, the substituted mono- or di-arylamino, the substituted mono- or di-heteroarylamino, the substituted alkylalkenylamino, the substituted alkylarylamino, the substituted alkylheteroarylamino, the substituted alkenylarylamino, the substituted alkenylheteroarylamino, or the substituted arylheteroarylamino in the formulas of the present disclosure, each independently are substituted by at least one selected from the group consisting of deuterium: halogen; cyano; carboxyl; nitro; hydroxy; phosphine oxide; (C1-C30)alkyl, halo(C1-C30)alkyl, (C2-C30)alkenyl, (C2-C30)alkynyl, (C1-C30)alkoxy, (C1-C30)alkylthio, (C3-C30)cycloalkyl, (C3-C30)cycloalkenyl, (3- to 7-membered)heterocycloalkyl, (C6-C30)aryloxy, (C6-C30)arylthio, (5- to 30-membered)heteroaryl unsubstituted or substituted with (C6-C30)aryl, (C6-C30)aryl unsubstituted or substituted with (5- to 30-membered)heteroaryl, tri(C1-C30)alkylsilyl, tri(C6-C30)arylsilyl, di(C1-C30)alkyl(C6-C30)arylsilyl, (C1-C30)alkyldi(C6-C30)arylsilyl, amino, mono- or di-(C1-C30)alkylamino, mono- or di-(C6-C30)arylamino unsubstituted or substituted with (C1-C30)alkyl, (C1-C30)alkyl(C6-C30)arylamino, (C1-C30)alkylcarbonyl, (C1-C30)alkoxycarbonyl, (C6-C30)arylcarbonyl, (C6-C30)arylphosphinyl, di(C6-C30)arylboronyl, di(C1-C30)alkylboronyl, (C1-C30)alkyl(C6-C30)arylboronyl, (C6-C30)ar(C1-C30)alkyl, and (C1-C30)alkyl(C6-C30)aryl. For example, the substituted group may be substituted with at least one of deuterium, phenyl unsubstituted or substituted with phenanthroline, naphthyl unsubstituted or substituted with phenyl, o-biphenyl, m-biphenyl, p-biphenyl, dimethylfluorenyl, diphenyfluorenyl, pyridyl, dibenzofuranyl, or dibenzothiophenyl, etc.

In formulas of the present disclosure, when a plurality of substituents represented by the same symbol are present, each of the substituents represented by the same symbol may be the same or different.

The same reference numerals refer to the same elements herein. In addition, in the drawings, the thickness, ratio, and dimensions of the elements are only exaggerated for effective description of technical content, and this does not change the technical content.

Hereinafter, an organic electroluminescent device according to one embodiment will be described.

According to one embodiment, an organic electroluminescent device comprising a deuterated compound is provided. Specifically, an organic electroluminescent device according to one embodiment comprises a plurality of light-emitting units positioned between the first electrode and the second electrode; and at least one charge generation layer positioned between the adjacent light-emitting units, wherein the light-emitting units comprise at least one light-emitting layer, and at least one of the light-emitting layer and the charge generation layer comprises a deuterated compound.

According to one embodiment, the degree of deuteriumization of the deuterated compounds contained in at least one of the light-emitting layer and the charge generation layer may be 30% to 100%, preferably 40% to 100%, more preferably 50% to 100%, even more preferably 60% to 100%. When deuterated with a number equal to or higher than the lower limit, the bond dissociation energy according to deuteration increases, thereby increasing the stability of the compound. When such a compound is used in an organic electroluminescent device, improved lifespan characteristics may be exhibited. Generally, the degree of improvement in lifespan characteristics increases as the degree of deuteriumization increases. However, if the degree of deuteriumization exceeds a certain percentage, the degree of improvement in lifespan characteristics may no longer increase.

The light-emitting layer according to one embodiment includes an organic electroluminescent compound represented by the following Formula 1-1.

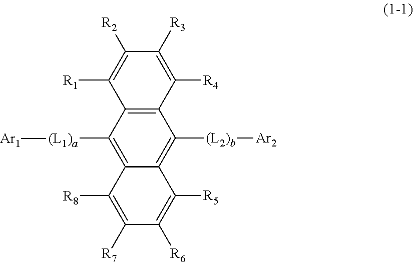

In Formula 1-1,
L₁ and L₂ each independently represent, a single bond, a substituted or unsubstituted (C6-C30)arylene, or a substituted or unsubstituted (3- to 30-membered)heteroarylene;
Ar₁ and Ar₂ each independently represent, a substituted or unsubstituted (C6-C30)aryl or a substituted or unsubstituted (3- to 30-membered)heteroaryl;
R₁ to R₆ each independently represent, hydrogen, deuterium, halogen, cyano, a substituted or unsubstituted (C1-C30)alkyl, a substituted or unsubstituted (C2-C30)alkenyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring of (C3-C30) aliphatic ring and (C6-C30) aromatic ring, a substituted or unsubstituted mono- or di-(C1-C30)alkylamino, a substituted or unsubstituted mono- or di-(C2-C30)alkenylamino, a substituted or unsubstituted mono- or di-(C6-C30)arylamino, a substituted or unsubstituted mono- or di-(3- to 30-membered)heteroarylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino; and
a and b each independently represent, an integer of 1 or 2;
when a and b are 2 or more, each of L₁ and L₂ may be the same or different.

In one embodiment, L₁ and L₂ each independently may be, a single bond, a substituted or unsubstituted (C6-C30)arylene, or a substituted or unsubstituted (5- to 30-membered)heteroarylene, preferably a single bond, a substituted or unsubstituted (C6-C25)arylene, or a substituted or unsubstituted (5- to 25-membered)heteroarylene, more preferably a single bond, a substituted or unsubstituted (C6-C18)arylene, or a substituted or unsubstituted (5- to 18-membered)heteroarylene. For example, L₁ and L₂ each independently may be, a single bond, phenylene unsubstituted or substituted with phenyl, naphthylene unsubstituted or substituted with phenyl, a substituted or unsubstituted phenanthrenylene, or a substituted or unsubstituted carbazolylene. Wherein, the substituted L₁ and L₂ may be substituted with deuterium.

In one embodiment, Ar₁ and Ar₂ each independently may be, a substituted or unsubstituted (C6-C30)aryl or a substituted or unsubstituted (5- to 30-membered)heteroaryl, preferably a substituted or unsubstituted (C6-C25)aryl or a substituted or unsubstituted (5- to 25-membered)heteroaryl. For example, Ar₁ and Ar₂ each independently may be, a substituted or unsubstituted phenyl, a substituted or unsubstituted naphthyl, a substituted or unsubstituted p-biphenyl, a substituted or unsubstituted m-biphenyl, a substituted or unsubstituted o-biphenyl, a substituted or unsubstituted p-terphenyl, a substituted or unsubstituted m-terphenyl, a substituted or unsubstituted phenanthrenyl, fluorenyl unsubstituted or substituted with at least one of methyl and phenyl, benzofluorenyl unsubstituted or substituted with at least one of methyl and phenyl, a substituted or unsubstituted spirobifluorenyl, a substituted or unsubstituted triphenylenyl, a substituted or unsubstituted C17 aryl, carbazolyl unsubstituted or substituted with phenyl, a substituted or unsubstituted dibenzofuranyl, a substituted or unsubstituted dibenzothiophenyl, a substituted or unsubstituted naphthobenzofuranyl, a substituted or unsubstituted naphthobenzothiophenyl, a substituted or unsubstituted epoxyphenanthrenyl, or a substituted or unsubstituted phenanthrothiophenyl. Wherein, the substituted Ar₁ and Ar₂ may be substituted with deuterium.

In one embodiment, R₁ to R₈ each independently may be, hydrogen or deuterium.

According to one embodiment, if one compound represented by Formula 1-1 comprises deuterium, the compound comprises at least one deuterium, preferably at least one of R₁ to R₆ may comprise deuterium, more preferably each of R₁ to R₈, and -(L₁)ₐ-Ar₁ may comprise at least one deuterium, even more preferably each of R₁ to R₈, -(L₁)ₐ-Ar₁, and -(L₂)_{b}-Ar₂ may comprise at least one deuterium. According to one embodiment, if one compound represented by Formula 1-1 comprises deuterium, the degree of deuteriumization is preferably at least 30% of the total hydrogen, more preferably at least 40% of the total hydrogen, even more preferably at least 50% of the total hydrogen. When deuterated the compound of Formula 1-1 with a number equal to or higher than the lower limit, the bond dissociation energy according to deuteration increases, thereby increasing the stability of the compound. When such a compound is used in an organic electroluminescent device, improved lifespan characteristics may be exhibited.

According to one embodiment, the compound represented by Formula 1-1 may be more specifically illustrated by the following compounds, but is not limited thereto.

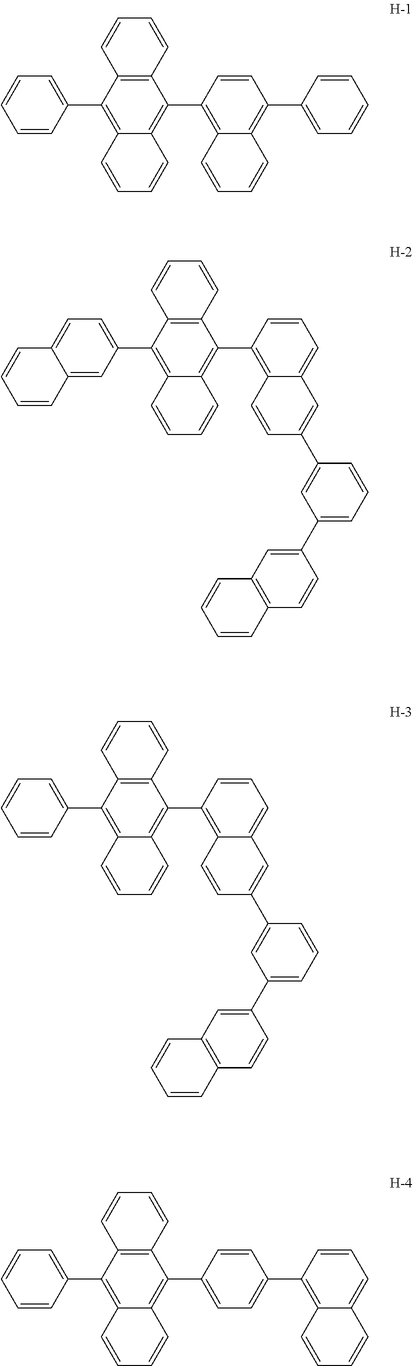

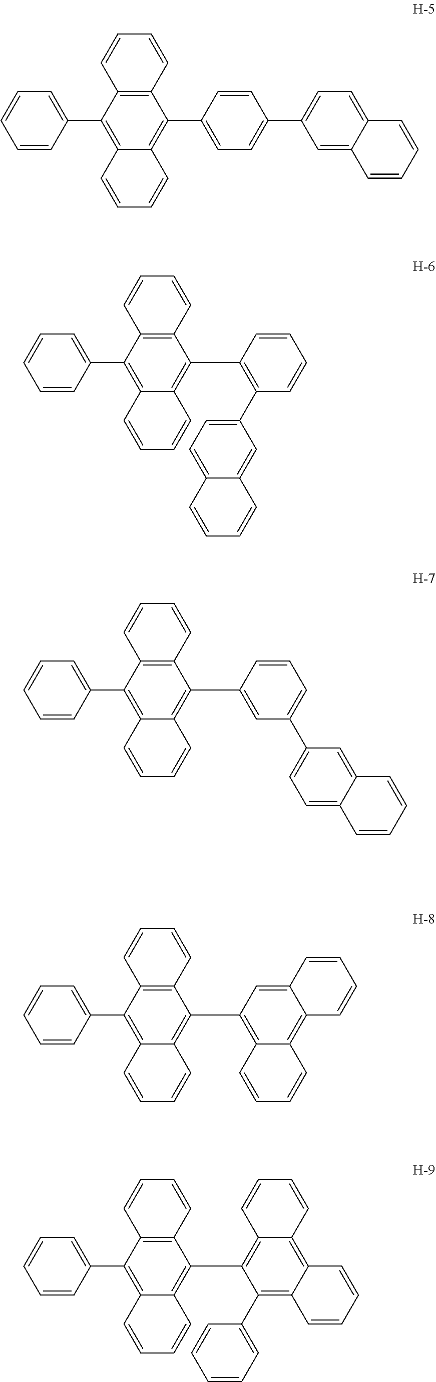

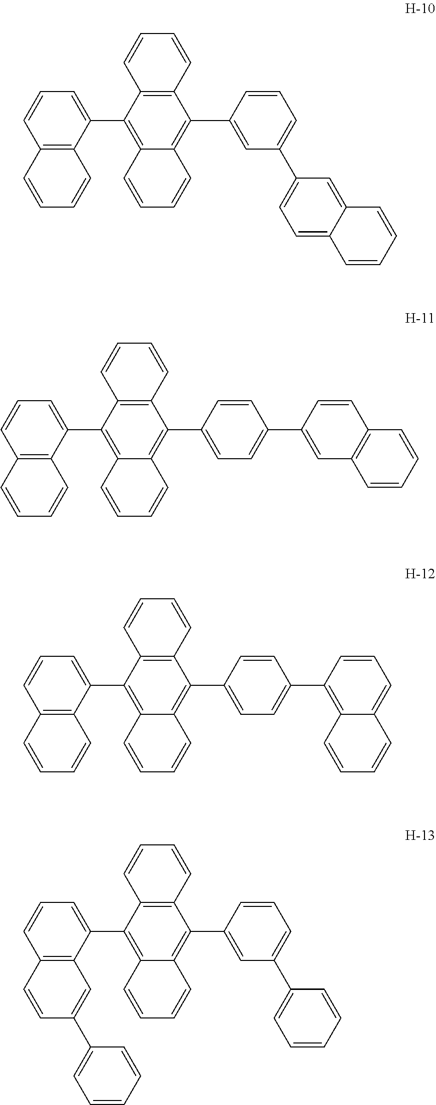

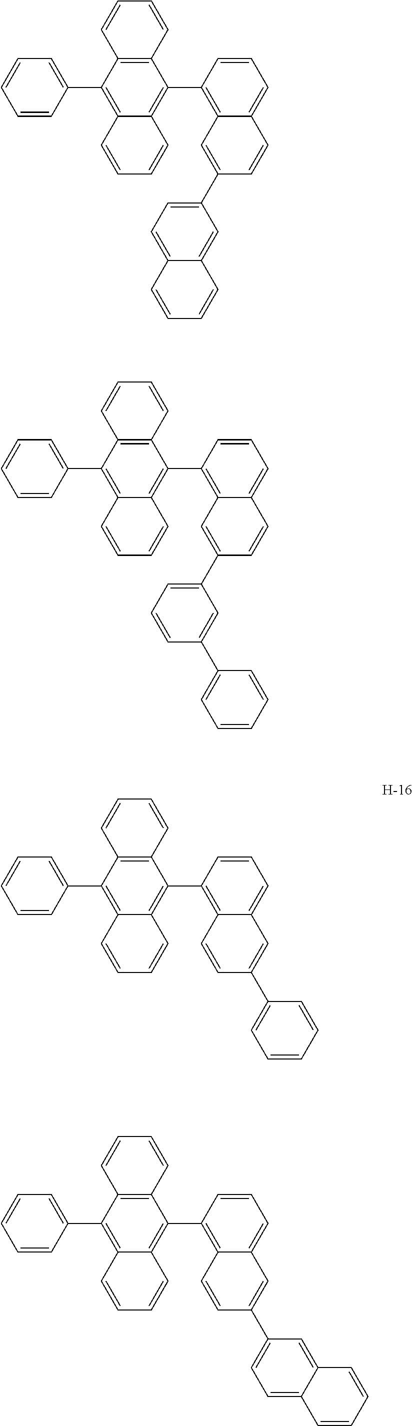

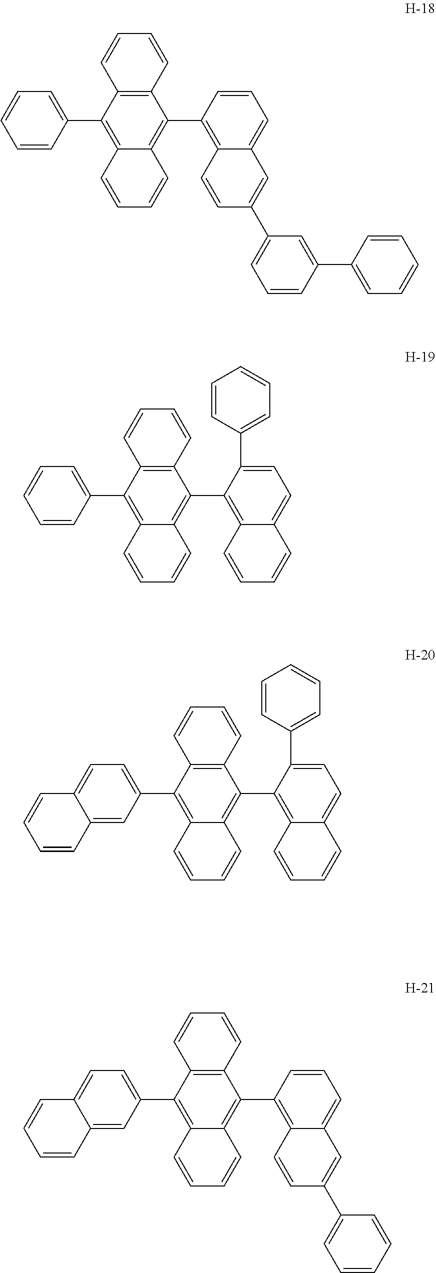

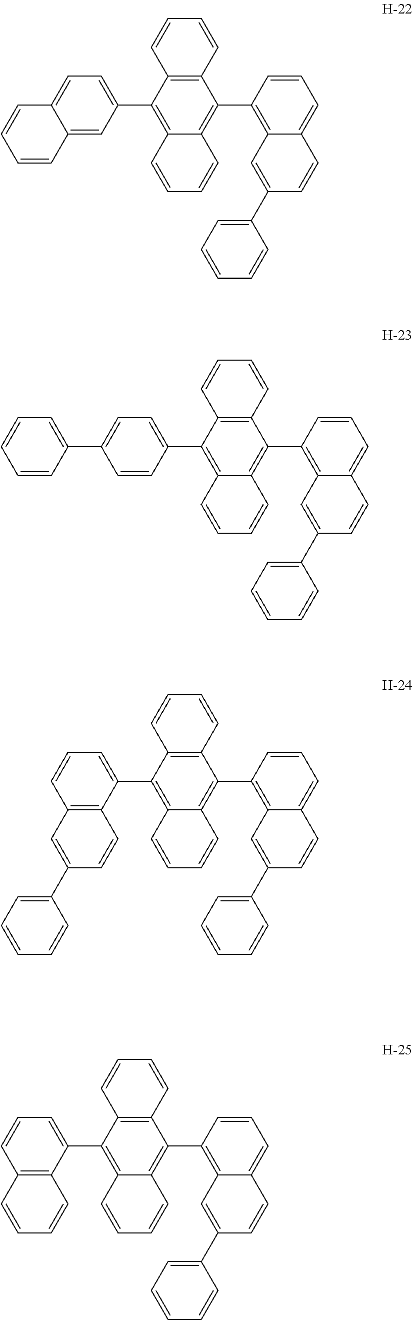

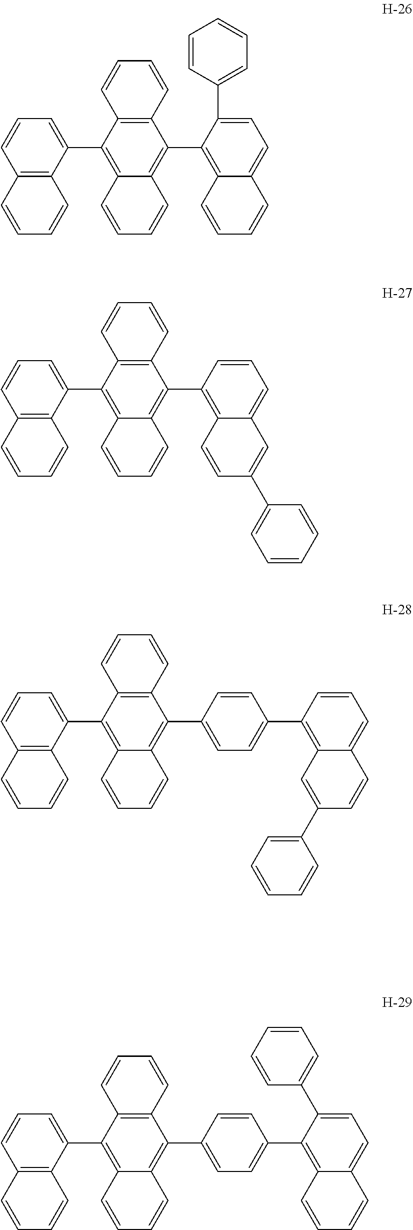

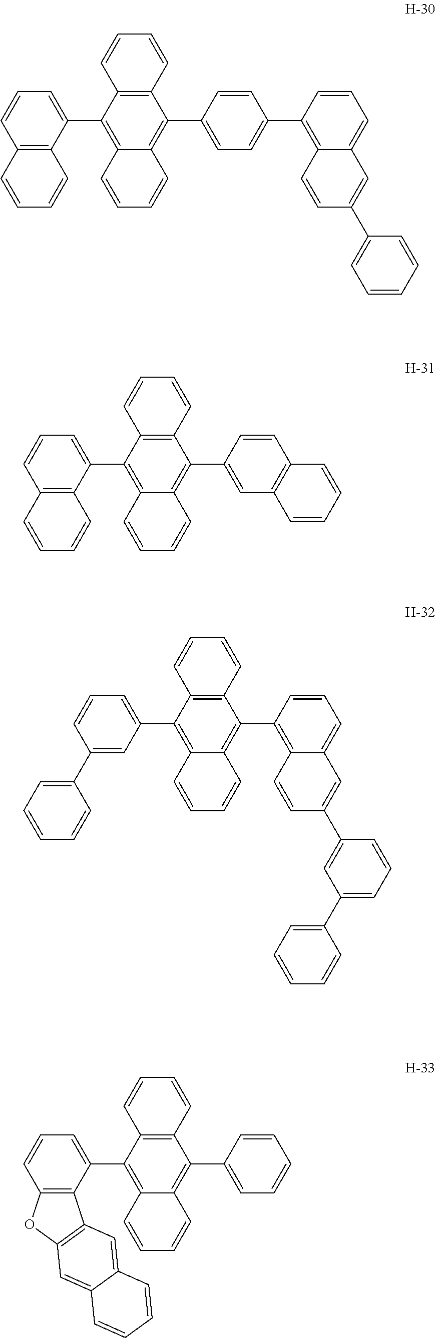

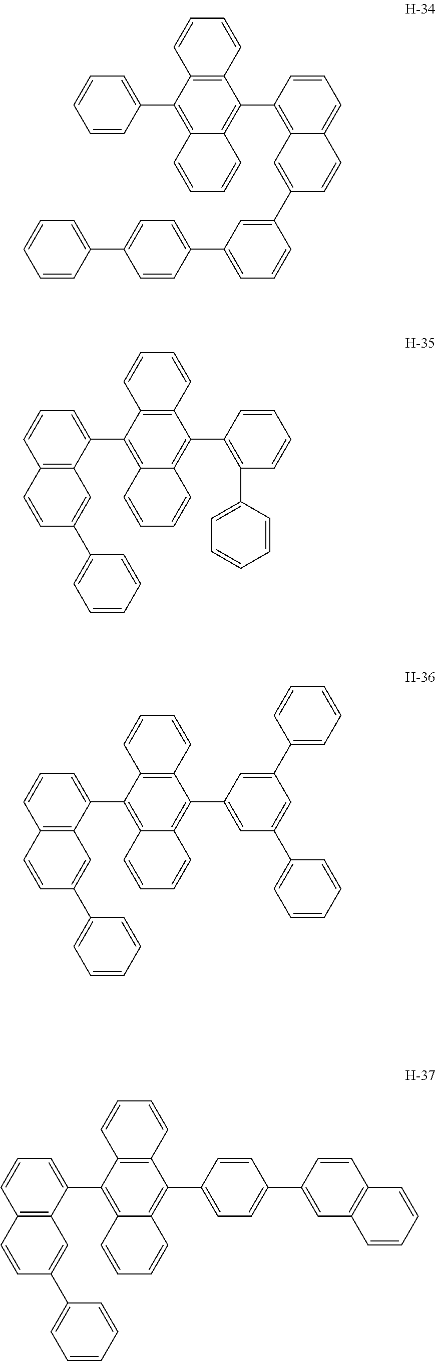

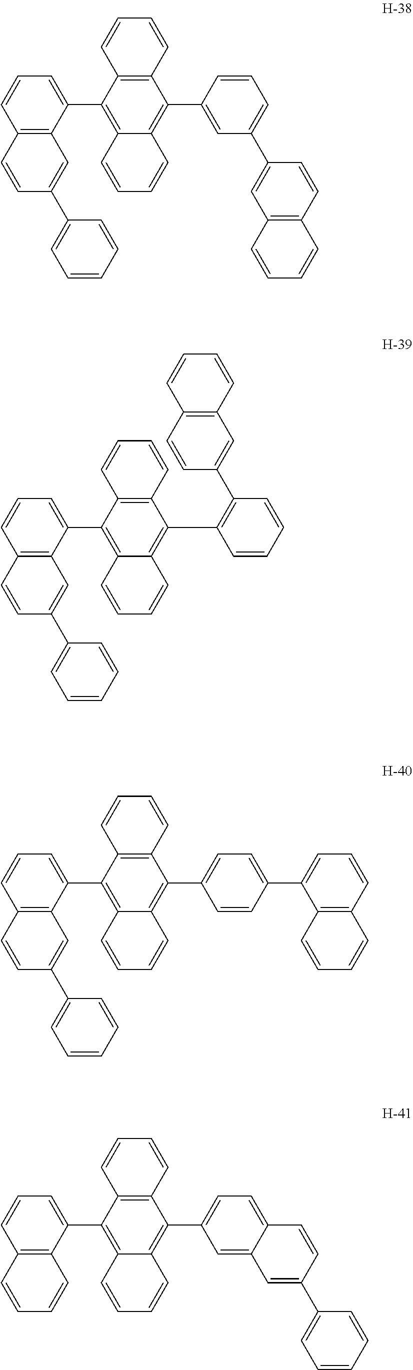

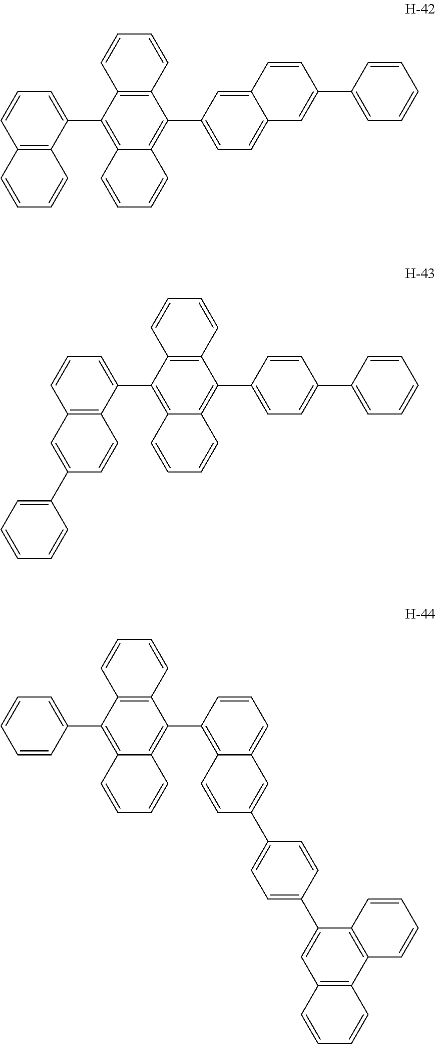

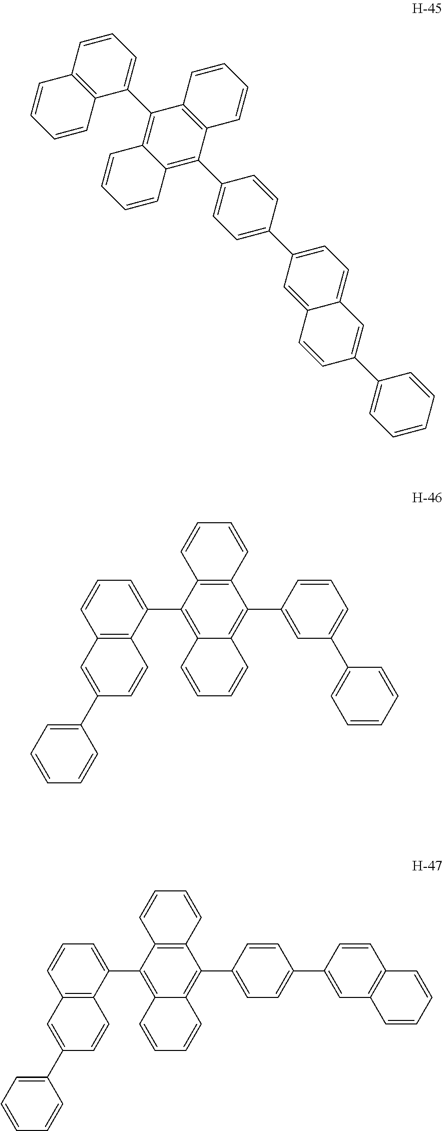

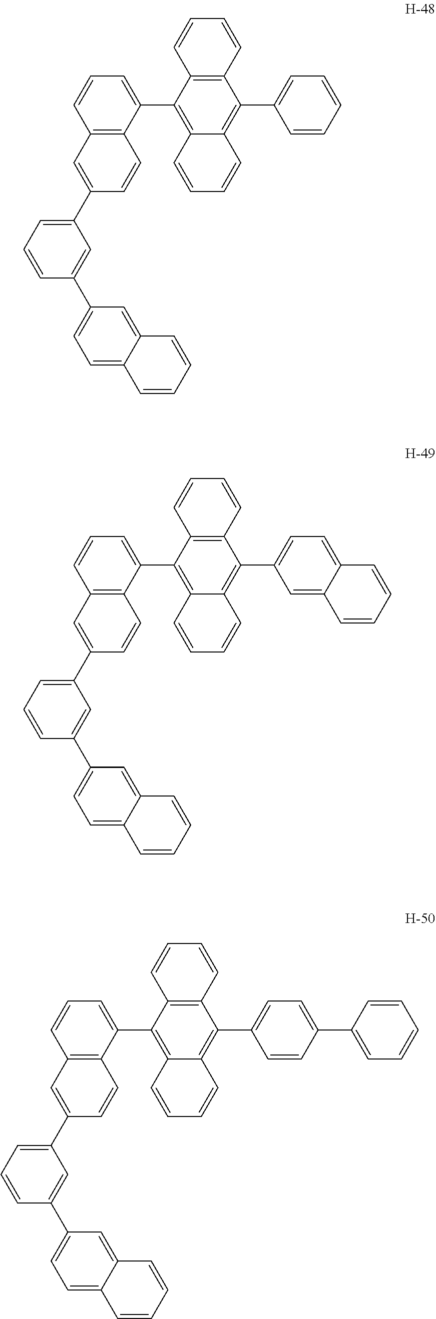

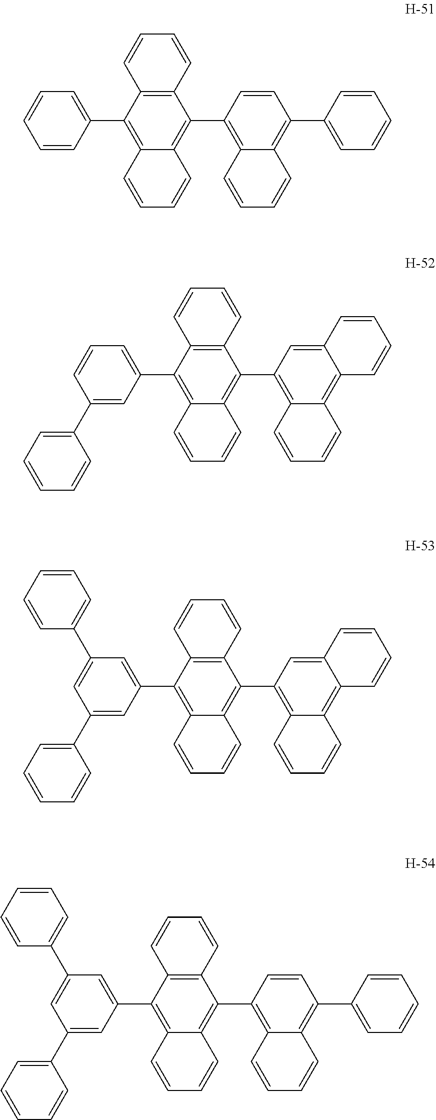

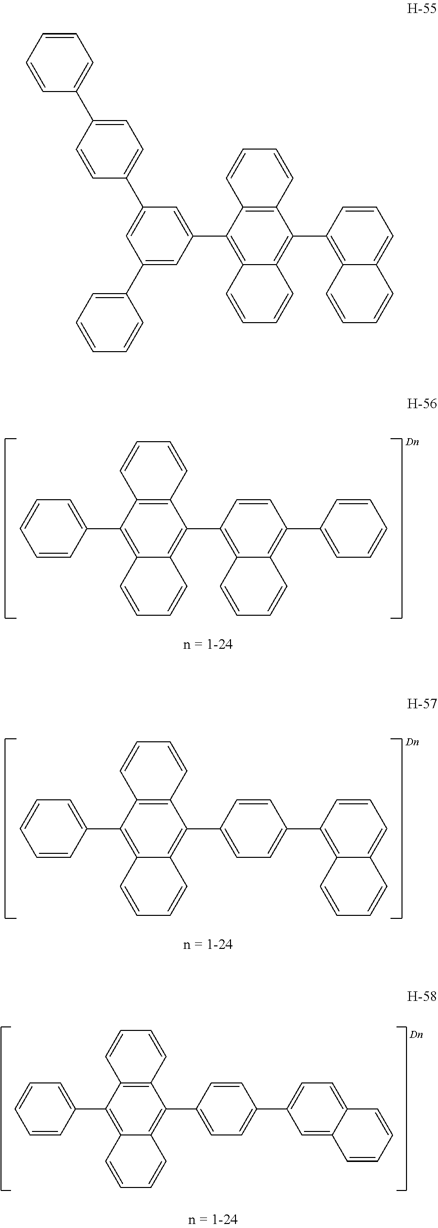

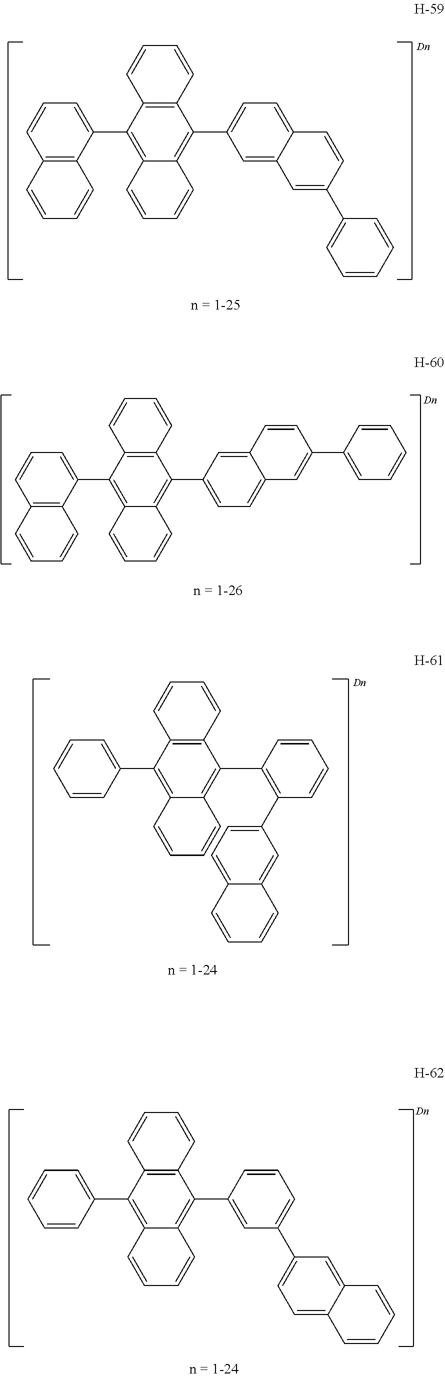

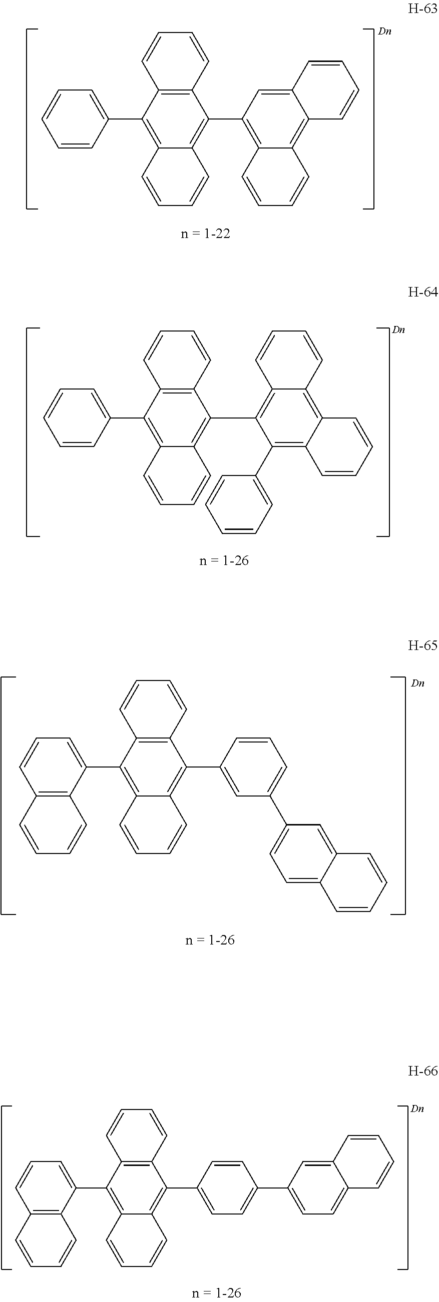

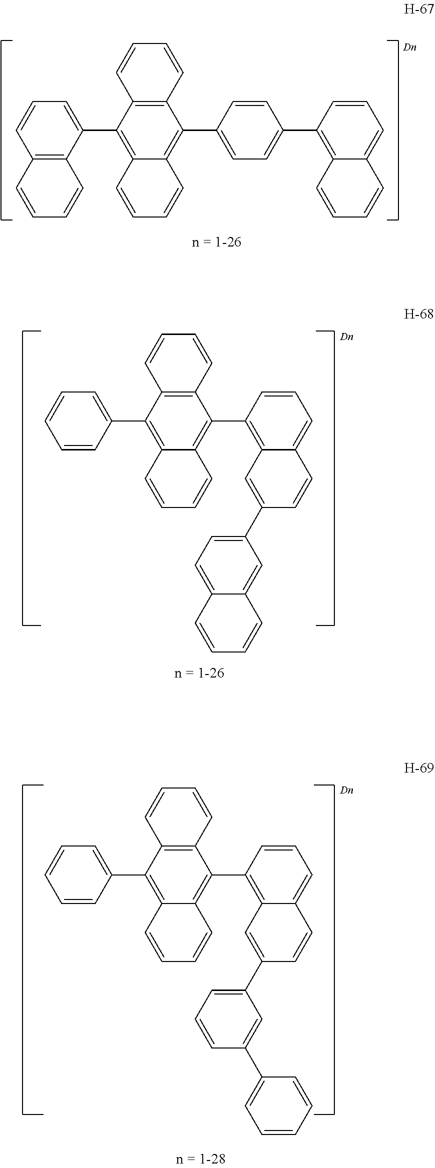

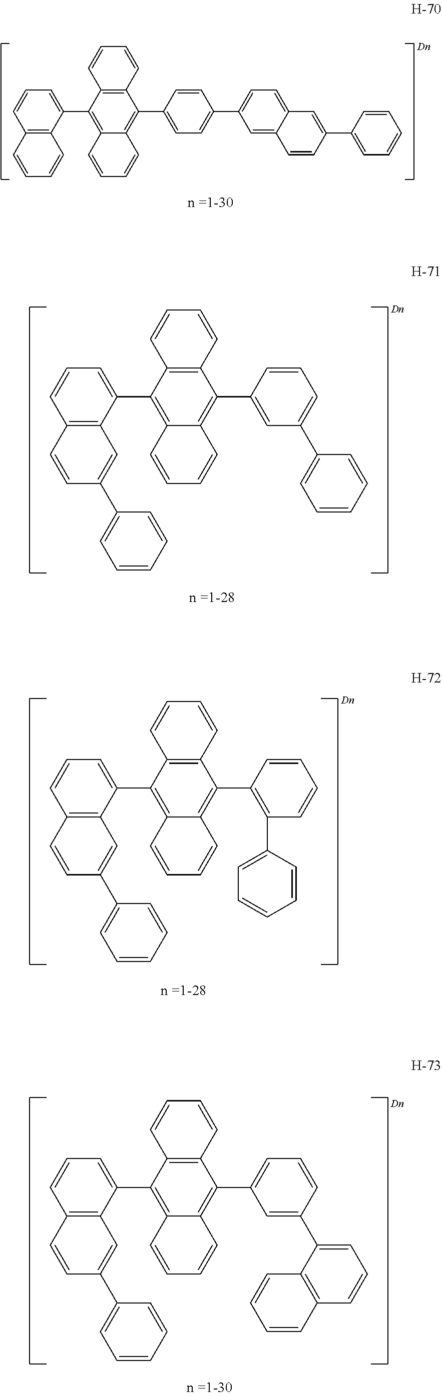

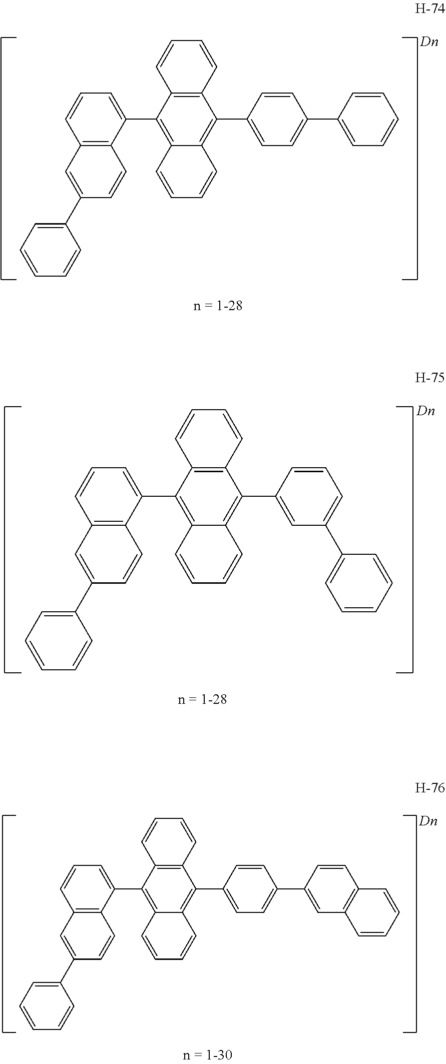

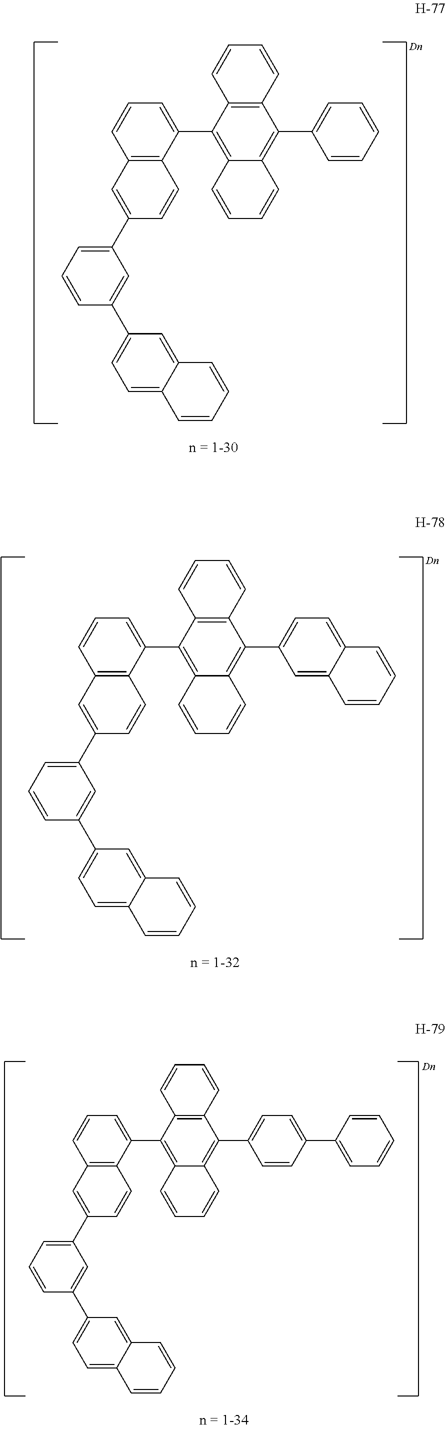

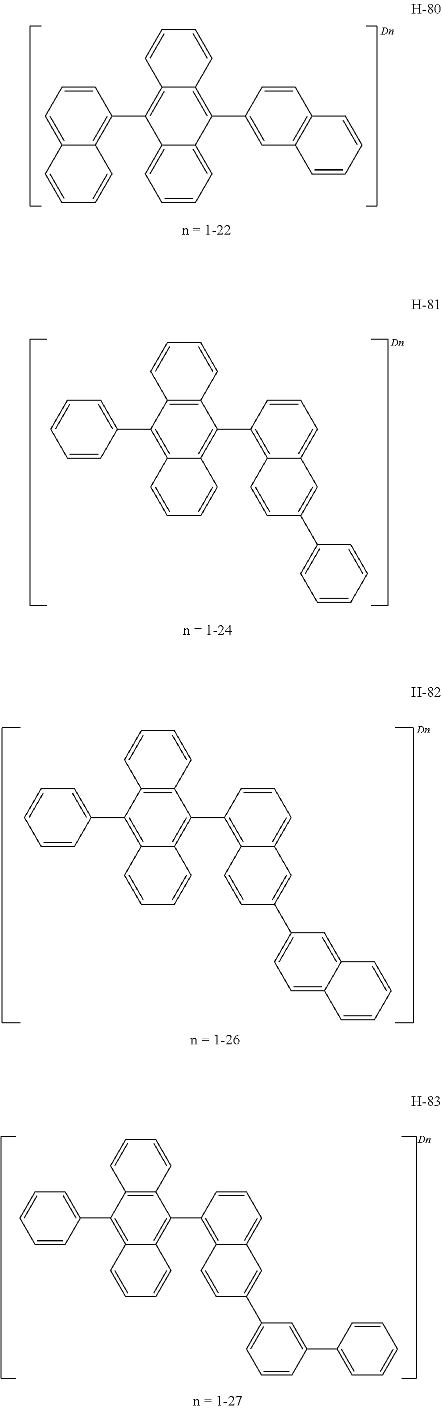

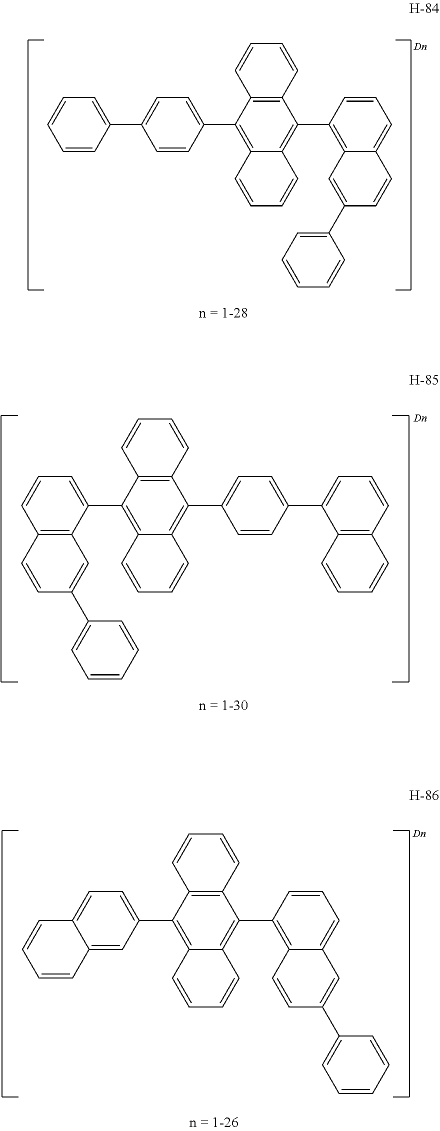

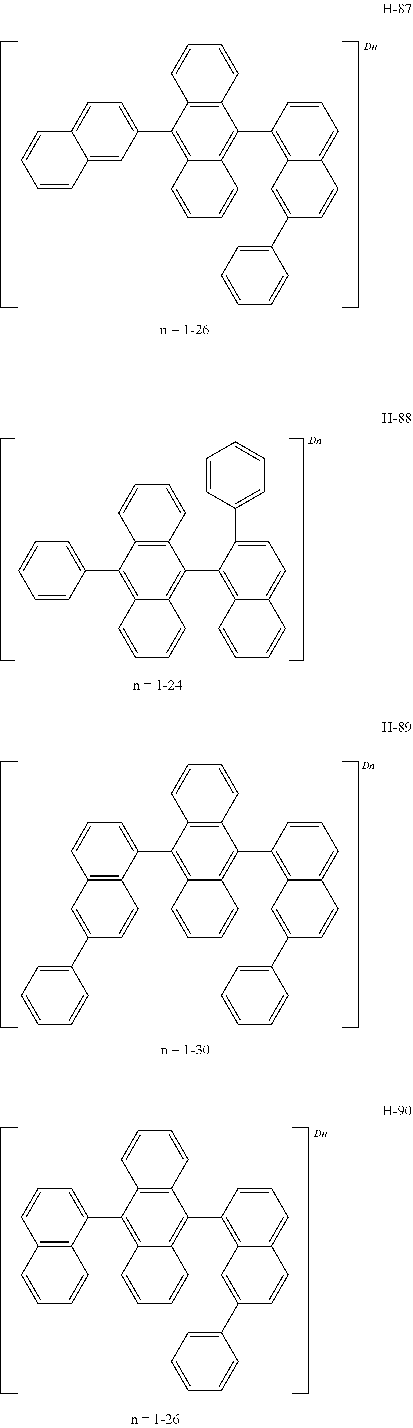

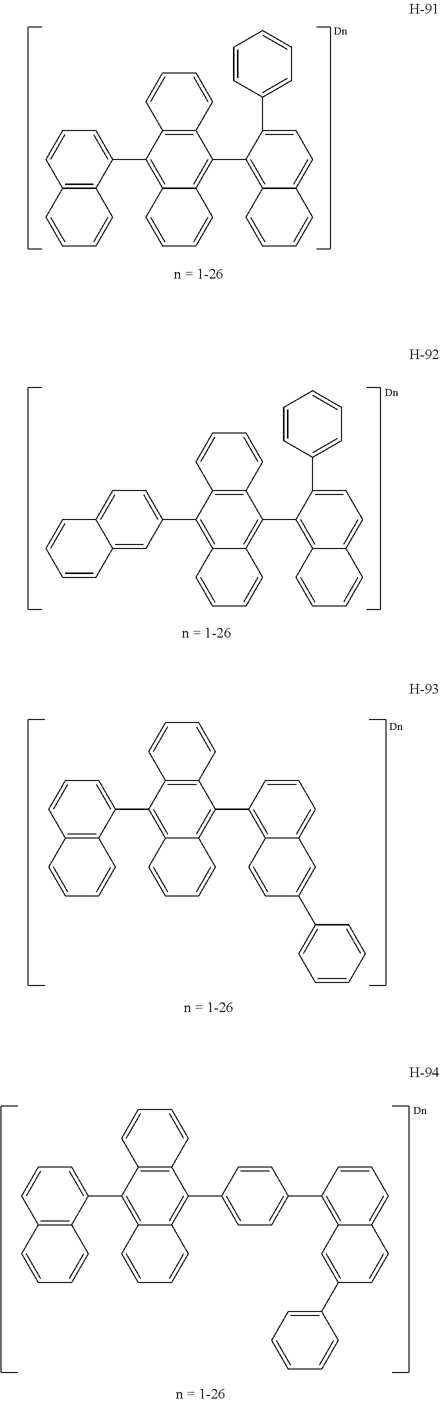

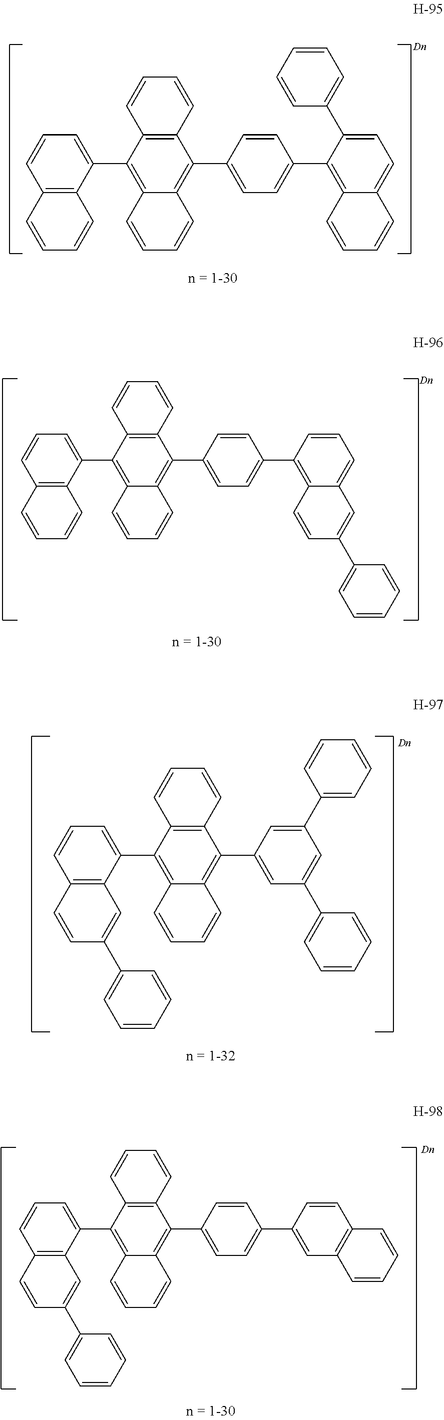

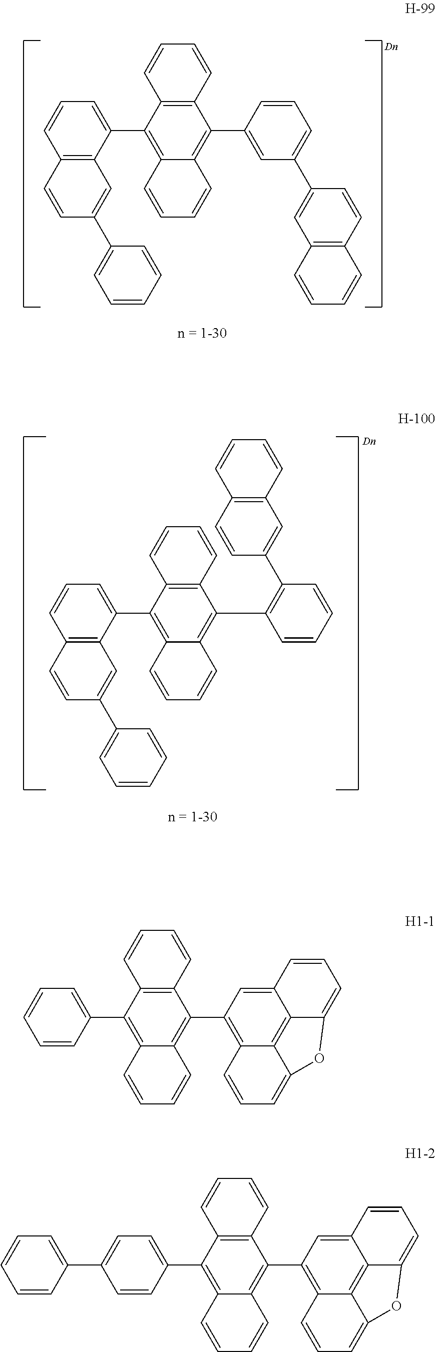

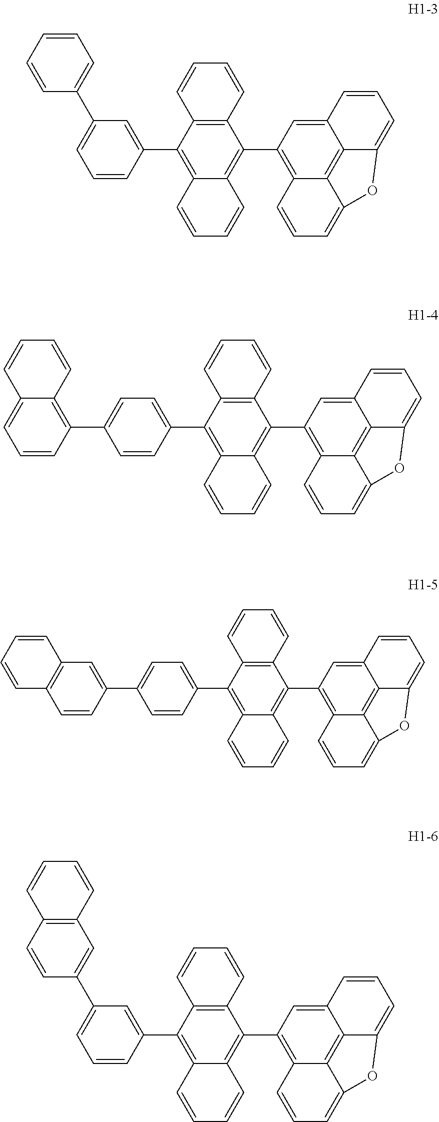 [SMILES: c1ccc(-c2cccc(-c3c4ccccc4c(-c4cc5cccc6oc7cccc4c7c56)c4ccccc34)c2)cc1 ; c1ccc2c(-c3ccc(-c4c5ccccc5c(-c5cc6cccc7oc8cccc5c8c67)c5ccccc45)cc3)cccc2c1 ; c1ccc2cc(-c3ccc(-c4c5ccccc5c(-c5cc6cccc7oc8cccc5c8c67)c5ccccc45)cc3)ccc2c1 ; c1cc(-c2ccc3ccccc3c2)cc(-c2c3ccccc3c(-c3cc4cccc5oc6cccc3c6c45)c3ccccc23)c1]

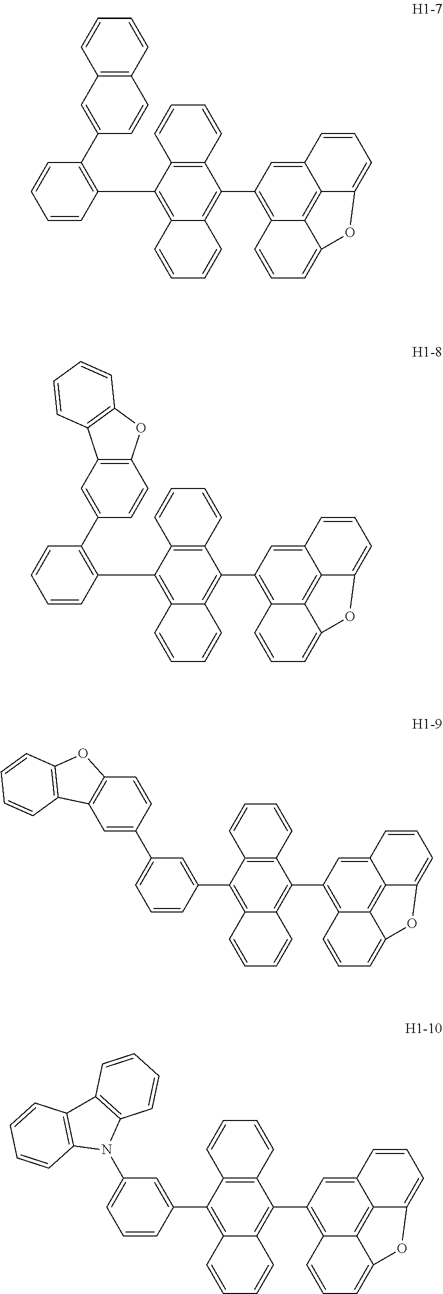 [SMILES: c1ccc(-c2c3ccccc3c(-c3cc4cccc5oc6cccc3c6c45)c3ccccc23)c(-c2ccc3ccccc3c2)c1 ; c1ccc(-c2c3ccccc3c(-c3cc4cccc5oc6cccc3c6c45)c3ccccc23)c(-c2ccc3oc4ccccc4c3c2)c1 ; c1cc(-c2ccc3oc4ccccc4c3c2)cc(-c2c3ccccc3c(-c3cc4cccc5oc6cccc3c6c45)c3ccccc23)c1 ; c1cc(-c2c3ccccc3c(-c3cc4cccc5oc6cccc3c6c45)c3ccccc23)cc(-n2c3ccccc3c3ccccc32)c1]

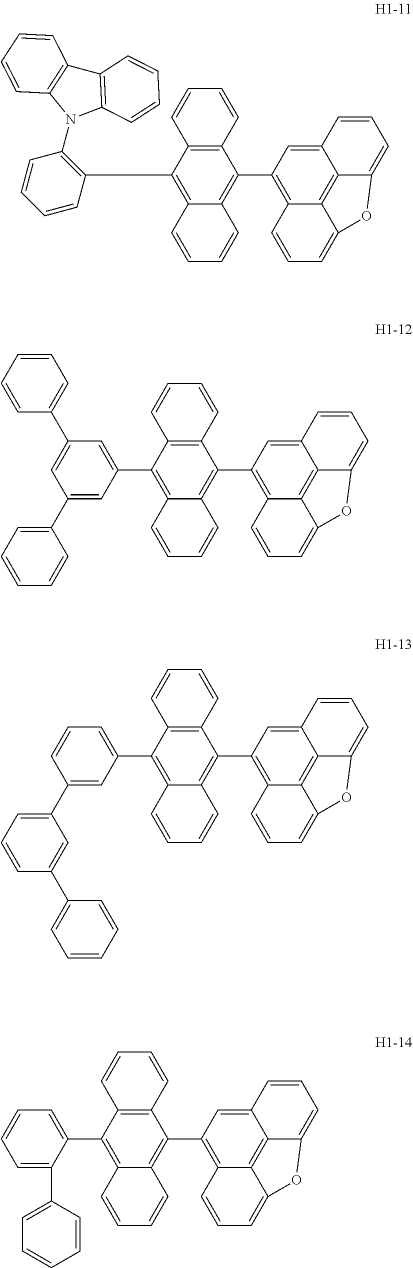 [SMILES: c1ccc(-n2c3ccccc3c3ccccc32)c(-c2c3ccccc3c(-c3cc4cccc5oc6cccc3c6c45)c3ccccc23)c1 ; c1ccc(-c2cc(-c3ccccc3)cc(-c3c4ccccc4c(-c4cc5cccc6oc7cccc4c7c56)c4ccccc34)c2)cc1 ; c1ccc(-c2cccc(-c3cccc(-c4c5ccccc5c(-c5cc6cccc7oc8cccc5c8c67)c5ccccc45)c3)c2)cc1 ; c1ccc(-c2ccccc2-c2c3ccccc3c(-c3cc4cccc5oc6cccc3c6c45)c3ccccc23)cc1]

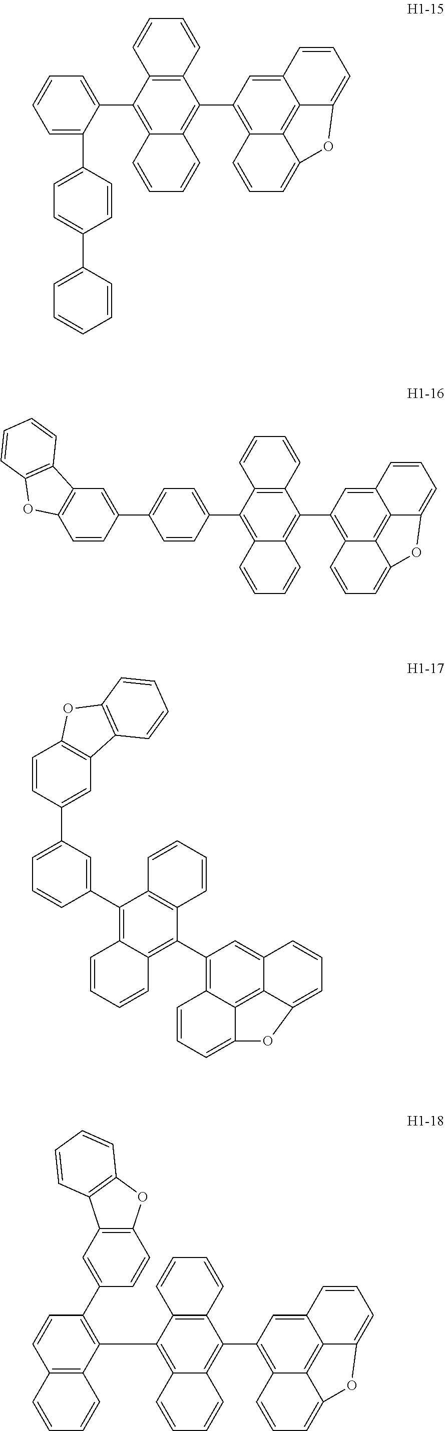 [SMILES: c1ccc(-c2ccc(-c3ccccc3-c3c4ccccc4c(-c4cc5cccc6oc7cccc4c7c56)c4ccccc34)cc2)cc1 ; c1ccc2c(c1)oc1ccc(-c3ccc(-c4c5ccccc5c(-c5cc6cccc7oc8cccc5c8c67)c5ccccc45)cc3)cc12 ; c1cc(-c2ccc3oc4ccccc4c3c2)cc(-c2c3ccccc3c(-c3cc4cccc5oc6cccc3c6c45)c3ccccc23)c1 ; c1ccc2c(-c3c4ccccc4c(-c4cc5cccc6oc7cccc4c7c56)c4ccccc34)c(-c3ccc4oc5ccccc5c4c3)ccc2c1]

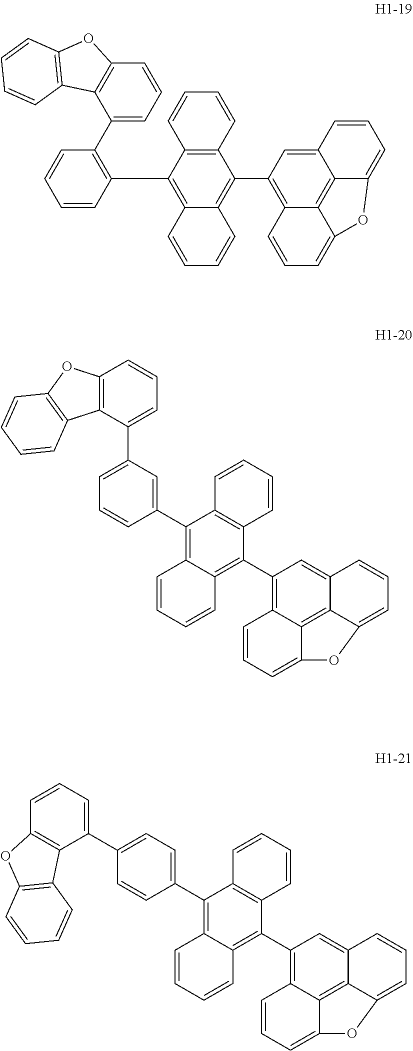 [SMILES: c1ccc(-c2cccc3oc4ccccc4c23)c(-c2c3ccccc3c(-c3cc4cccc5oc6cccc3c6c45)c3ccccc23)c1 ; c1cc(-c2c3ccccc3c(-c3cc4cccc5oc6cccc3c6c45)c3ccccc23)cc(-c2cccc3oc4ccccc4c23)c1 ; c1ccc2c(c1)oc1cccc(-c3ccc(-c4c5ccccc5c(-c5cc6cccc7oc8cccc5c8c67)c5ccccc45)cc3)c12]

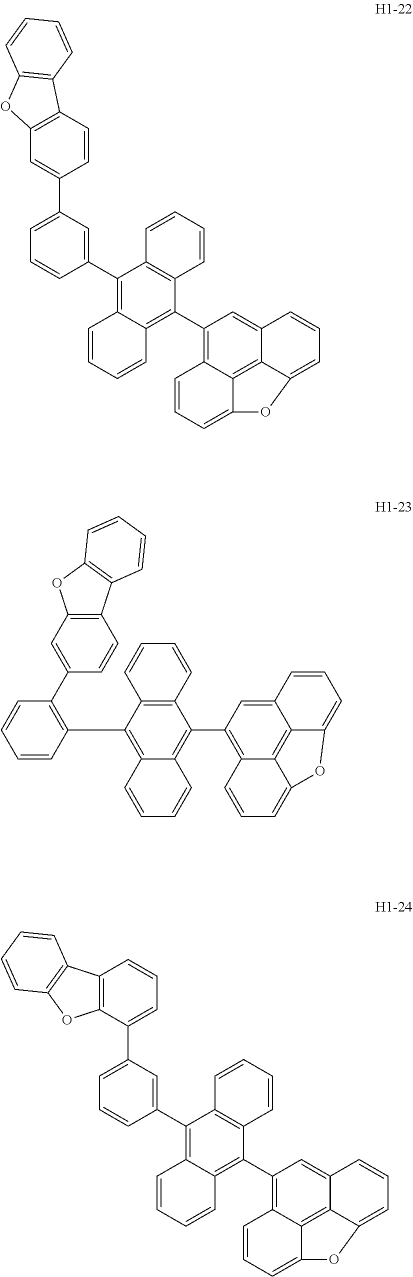 [SMILES: c1cc(-c2ccc3c(c2)oc2ccccc23)cc(-c2c3ccccc3c(-c3cc4cccc5oc6cccc3c6c45)c3ccccc23)c1 ; c1ccc(-c2c3ccccc3c(-c3cc4cccc5oc6cccc3c6c45)c3ccccc23)c(-c2ccc3c(c2)oc2ccccc23)c1 ; c1cc(-c2c3ccccc3c(-c3cc4cccc5oc6cccc3c6c45)c3ccccc23)cc(-c2cccc3c2oc2ccccc23)c1]

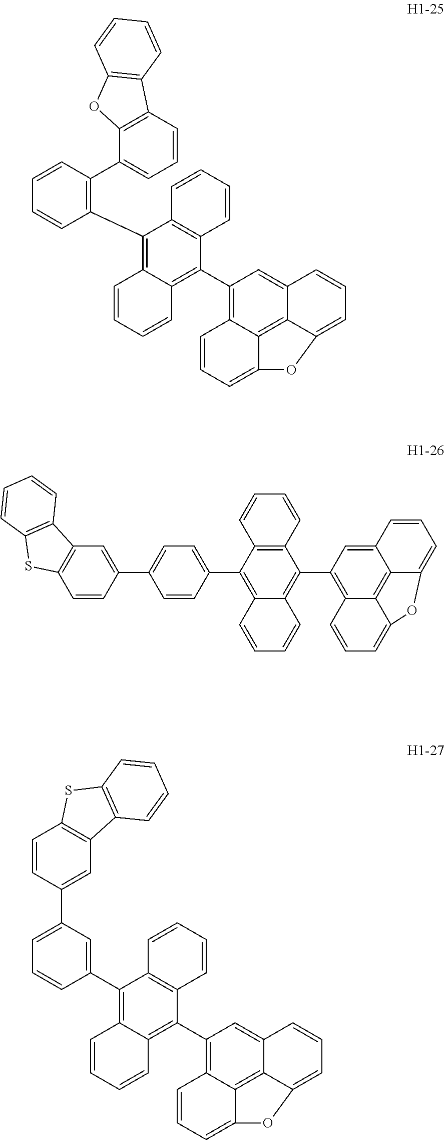 [SMILES: c1ccc(-c2cccc3c2oc2ccccc23)c(-c2c3ccccc3c(-c3cc4cccc5oc6cccc3c6c45)c3ccccc23)c1 ; c1cc2cc(-c3c4ccccc4c(-c4ccc(-c5ccc6sc7ccccc7c6c5)cc4)c4ccccc34)c3cccc4oc(c1)c2c43 ; c1cc(-c2ccc3sc4ccccc4c3c2)cc(-c2c3ccccc3c(-c3cc4cccc5oc6cccc3c6c45)c3ccccc23)c1]

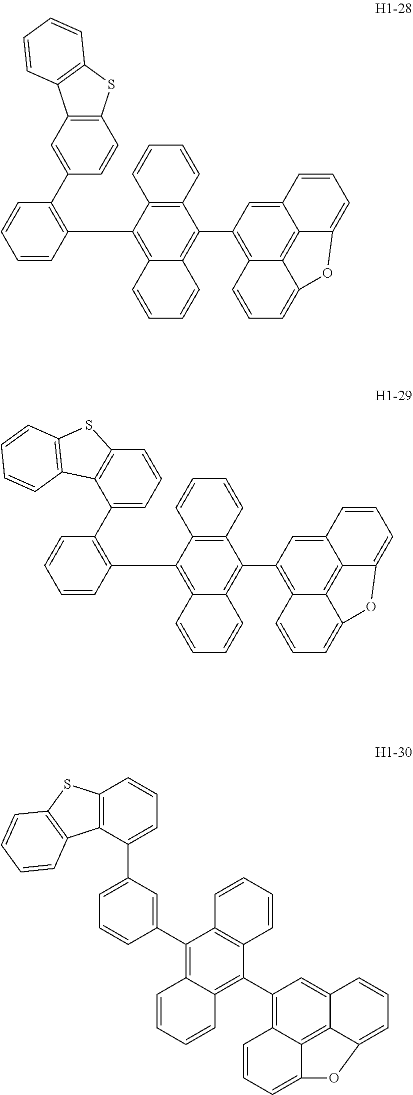 [SMILES: c1ccc(-c2c3ccccc3c(-c3cc4cccc5oc6cccc3c6c45)c3ccccc23)c(-c2ccc3sc4ccccc4c3c2)c1 ; c1ccc(-c2cccc3sc4ccccc4c23)c(-c2c3ccccc3c(-c3cc4cccc5oc6cccc3c6c45)c3ccccc23)c1 ; c1cc(-c2c3ccccc3c(-c3cc4cccc5oc6cccc3c6c45)c3ccccc23)cc(-c2cccc3sc4ccccc4c23)c1]

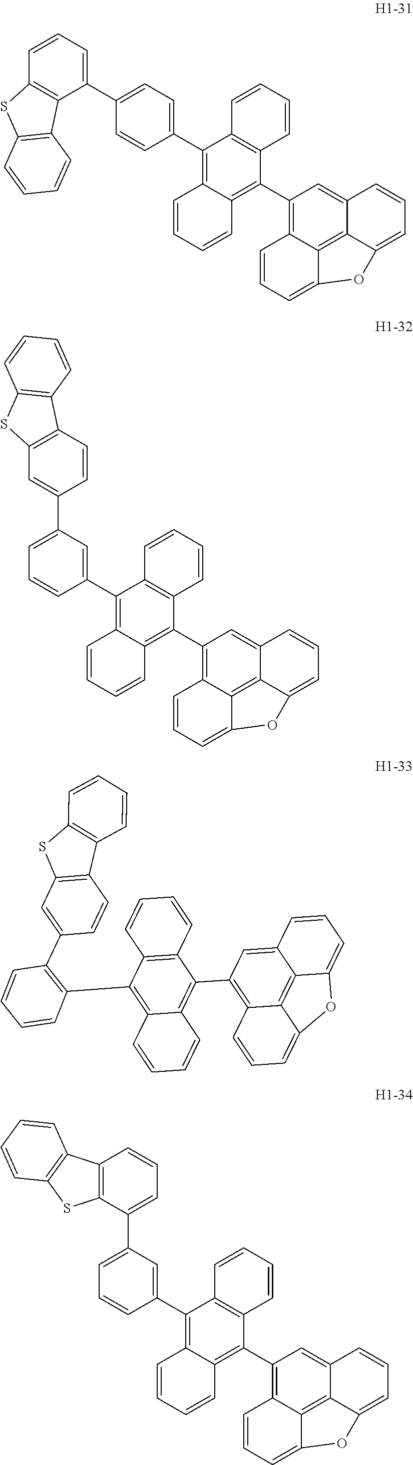 [SMILES: c1cc2cc(-c3c4ccccc4c(-c4ccc(-c5cccc6sc7ccccc7c56)cc4)c4ccccc34)c3cccc4oc(c1)c2c43 ; c1cc(-c2ccc3c(c2)sc2ccccc23)cc(-c2c3ccccc3c(-c3cc4cccc5oc6cccc3c6c45)c3ccccc23)c1 ; c1ccc(-c2c3ccccc3c(-c3cc4cccc5oc6cccc3c6c45)c3ccccc23)c(-c2ccc3c(c2)sc2ccccc23)c1 ; c1cc(-c2c3ccccc3c(-c3cc4cccc5oc6cccc3c6c45)c3ccccc23)cc(-c2cccc3c2sc2ccccc23)c1]

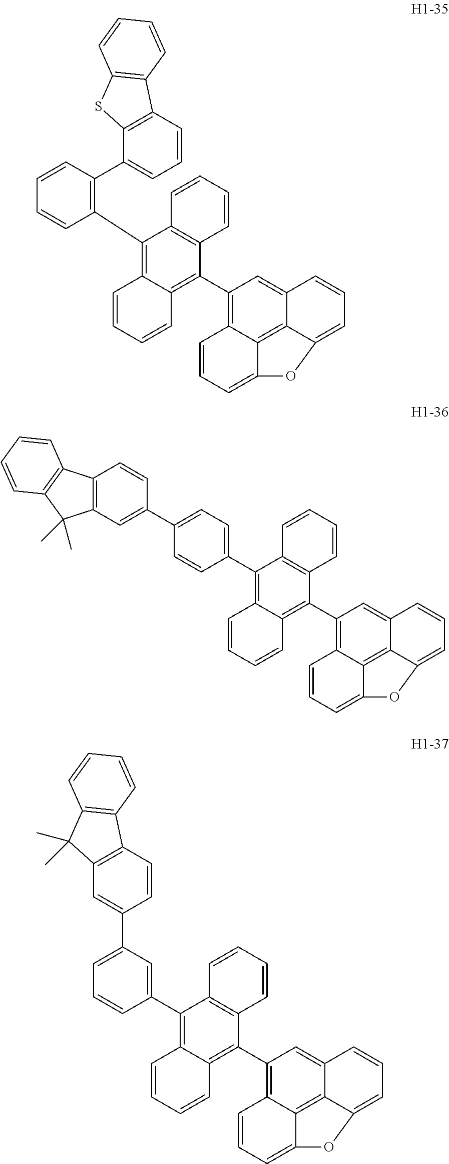 [SMILES: c1ccc(-c2cccc3c2sc2ccccc23)c(-c2c3ccccc3c(-c3cc4cccc5oc6cccc3c6c45)c3ccccc23)c1 ; CC1(C)c2ccccc2-c2ccc(-c3ccc(-c4c5ccccc5c(-c5cc6cccc7oc8cccc5c8c67)c5ccccc45)cc3)cc21 ; CC1(C)c2ccccc2-c2ccc(-c3cccc(-c4c5ccccc5c(-c5cc6cccc7oc8cccc5c8c67)c5ccccc45)c3)cc21]

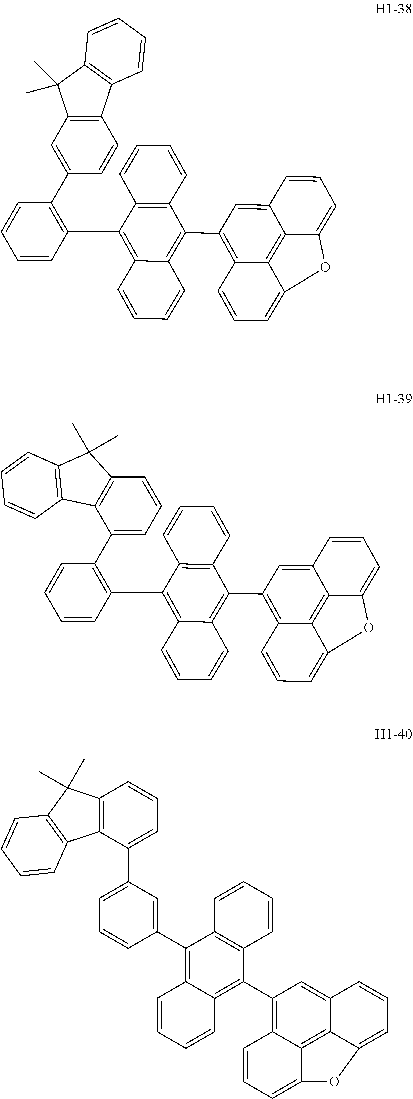 [SMILES: CC1(C)c2ccccc2-c2ccc(-c3ccccc3-c3c4ccccc4c(-c4cc5cccc6oc7cccc4c7c56)c4ccccc34)cc21 ; CC1(C)c2ccccc2-c2c(-c3ccccc3-c3c4ccccc4c(-c4cc5cccc6oc7cccc4c7c56)c4ccccc34)cccc21 ; CC1(C)c2ccccc2-c2c(-c3cccc(-c4c5ccccc5c(-c5cc6cccc7oc8cccc5c8c67)c5ccccc45)c3)cccc21]

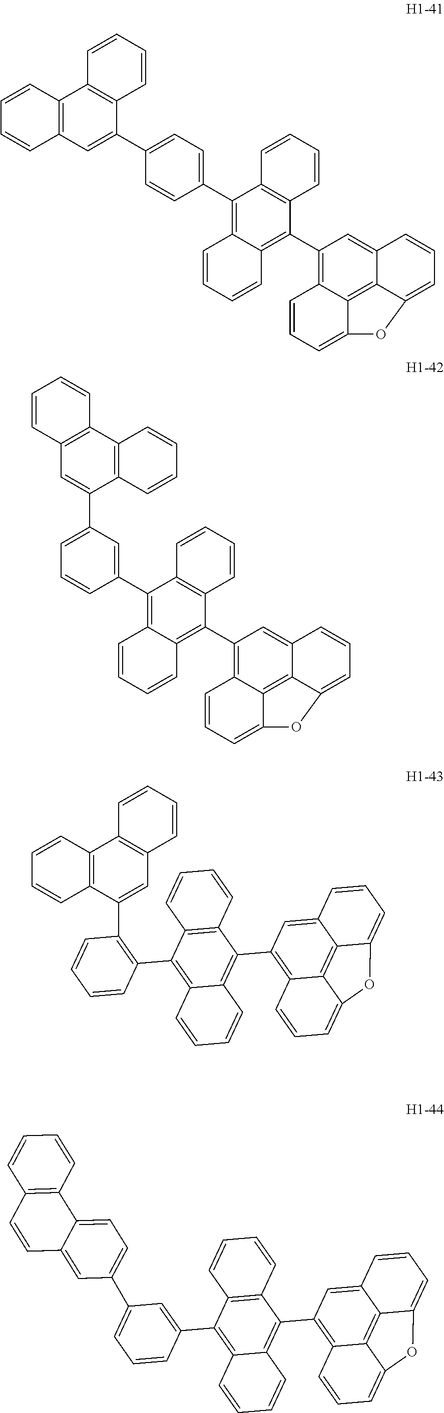 [SMILES: c1ccc2c(c1)cc(-c1ccc(-c3c4ccccc4c(-c4cc5cccc6oc7cccc4c7c56)c4ccccc34)cc1)c1ccccc12 ; c1cc(-c2c3ccccc3c(-c3cc4cccc5oc6cccc3c6c45)c3ccccc23)cc(-c2cc3ccccc3c3ccccc23)c1 ; c1ccc(-c2cc3ccccc3c3ccccc23)c(-c2c3ccccc3c(-c3cc4cccc5oc6cccc3c6c45)c3ccccc23)c1 ; c1cc(-c2ccc3c(ccc4ccccc43)c2)cc(-c2c3ccccc3c(-c3cc4cccc5oc6cccc3c6c45)c3ccccc23)c1]

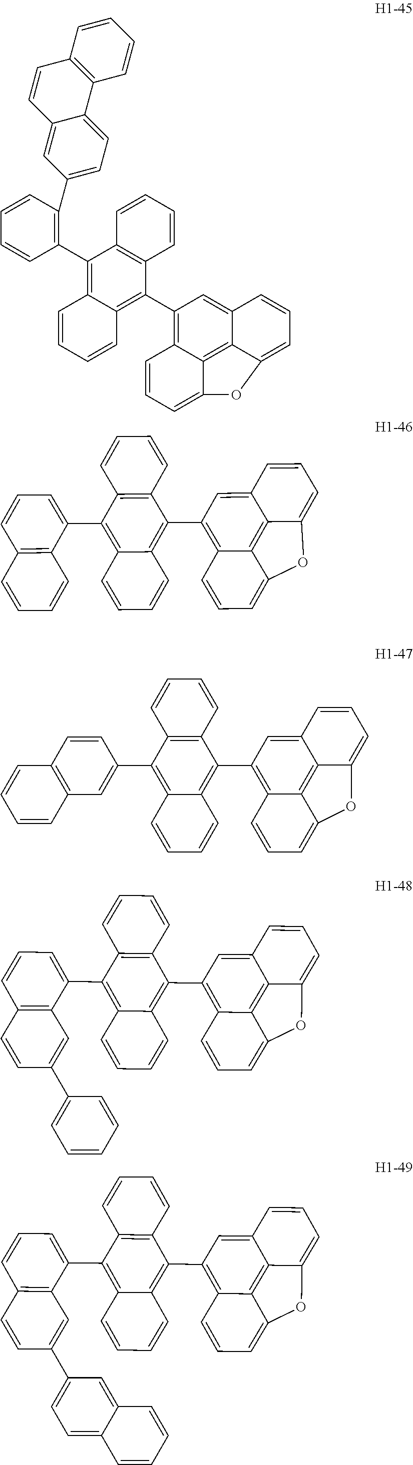

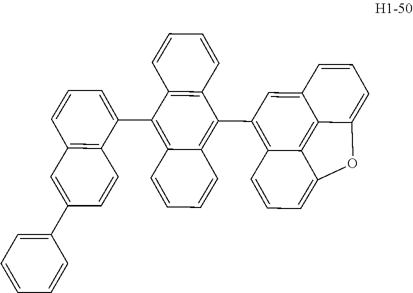 [SMILES: c1ccc(-c2ccc3c(-c4c5ccccc5c(-c5cc6cccc7oc8cccc5c8c67)c5ccccc45)cccc3c2)cc1]

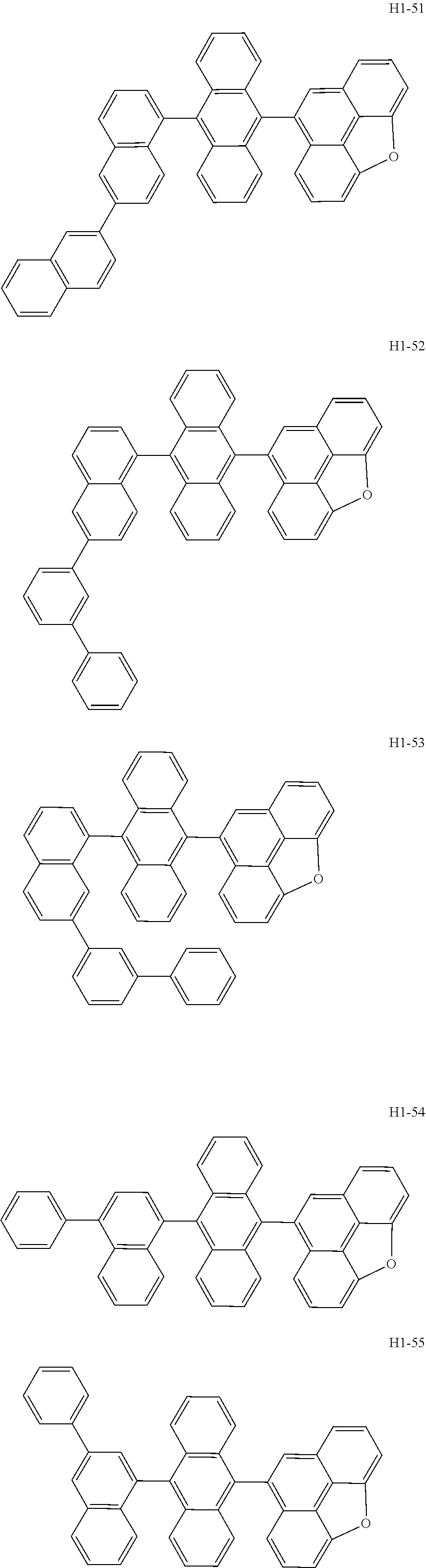 [SMILES: c1ccc2cc(-c3ccc4c(-c5c6ccccc6c(-c6cc7cccc8oc9cccc6c9c78)c6ccccc56)cccc4c3)ccc2c1 ; c1ccc(-c2cccc(-c3ccc4c(-c5c6ccccc6c(-c6cc7cccc8oc9cccc6c9c78)c6ccccc56)cccc4c3)c2)cc1 ; c1ccc(-c2cccc(-c3ccc4cccc(-c5c6ccccc6c(-c6cc7cccc8oc9cccc6c9c78)c6ccccc56)c4c3)c2)cc1 ; c1ccc(-c2ccc(-c3c4ccccc4c(-c4cc5cccc6oc7cccc4c7c56)c4ccccc34)c3ccccc23)cc1 ; c1ccc(-c2cc(-c3c4ccccc4c(-c4cc5cccc6oc7cccc4c7c56)c4ccccc34)c3ccccc3c2)cc1]

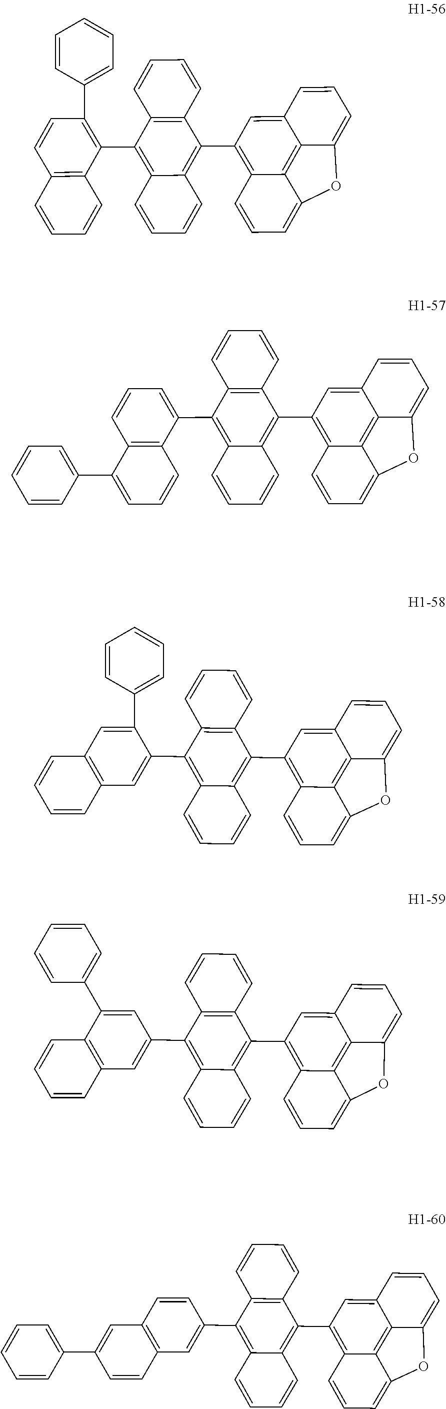 [SMILES: c1ccc(-c2ccc3ccccc3c2-c2c3ccccc3c(-c3cc4cccc5oc6cccc3c6c45)c3ccccc23)cc1 ; c1ccc(-c2cccc3c(-c4c5ccccc5c(-c5cc6cccc7oc8cccc5c8c67)c5ccccc45)cccc23)cc1 ; c1ccc(-c2cc3ccccc3cc2-c2c3ccccc3c(-c3cc4cccc5oc6cccc3c6c45)c3ccccc23)cc1 ; c1ccc(-c2cc(-c3c4ccccc4c(-c4cc5cccc6oc7cccc4c7c56)c4ccccc34)cc3ccccc23)cc1 ; c1ccc(-c2ccc3cc(-c4c5ccccc5c(-c5cc6cccc7oc8cccc5c8c67)c5ccccc45)ccc3c2)cc1]

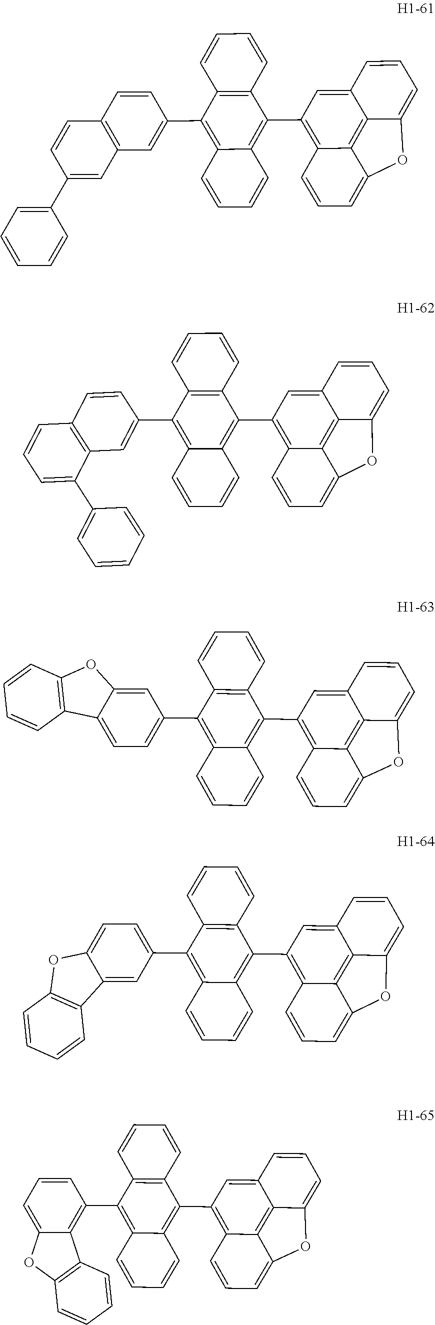 [SMILES: c1ccc(-c2ccc3ccc(-c4c5ccccc5c(-c5cc6cccc7oc8cccc5c8c67)c5ccccc45)cc3c2)cc1 ; c1ccc(-c2cccc3ccc(-c4c5ccccc5c(-c5cc6cccc7oc8cccc5c8c67)c5ccccc45)cc23)cc1 ; c1ccc2c(c1)oc1cc(-c3c4ccccc4c(-c4cc5cccc6oc7cccc4c7c56)c4ccccc34)ccc12 ; c1ccc2c(c1)oc1ccc(-c3c4ccccc4c(-c4cc5cccc6oc7cccc4c7c56)c4ccccc34)cc12 ; c1ccc2c(c1)oc1cccc(-c3c4ccccc4c(-c4cc5cccc6oc7cccc4c7c56)c4ccccc34)c12]

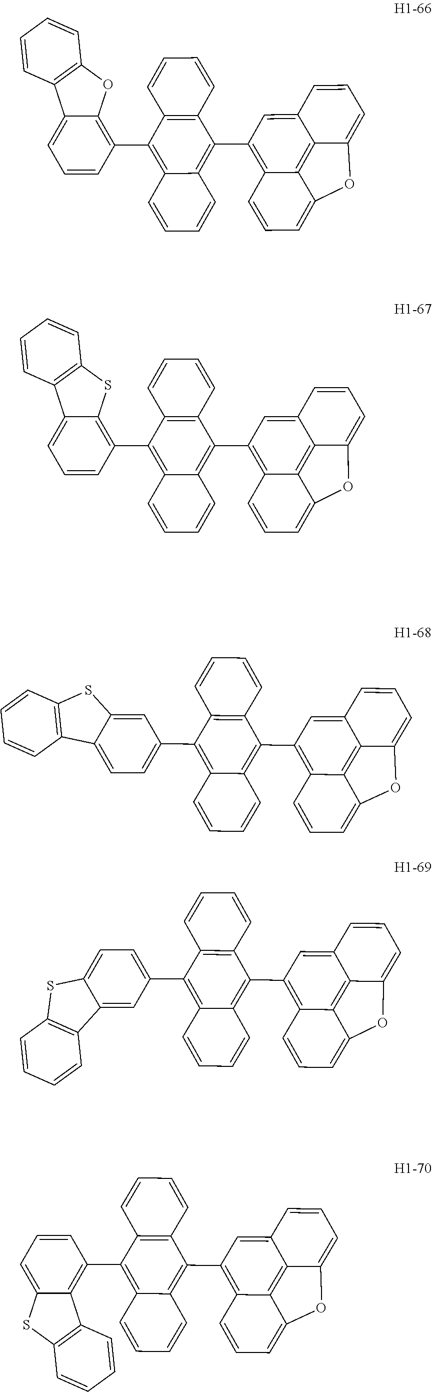 [SMILES: c1ccc2c(c1)oc1c(-c3c4ccccc4c(-c4cc5cccc6oc7cccc4c7c56)c4ccccc34)cccc12 ; c1cc2cc(-c3c4ccccc4c(-c4cccc5c4sc4ccccc45)c4ccccc34)c3cccc4oc(c1)c2c43 ; c1cc2cc(-c3c4ccccc4c(-c4ccc5c(c4)sc4ccccc45)c4ccccc34)c3cccc4oc(c1)c2c43 ; c1cc2cc(-c3c4ccccc4c(-c4ccc5sc6ccccc6c5c4)c4ccccc34)c3cccc4oc(c1)c2c43 ; c1cc2cc(-c3c4ccccc4c(-c4cccc5sc6ccccc6c45)c4ccccc34)c3cccc4oc(c1)c2c43]

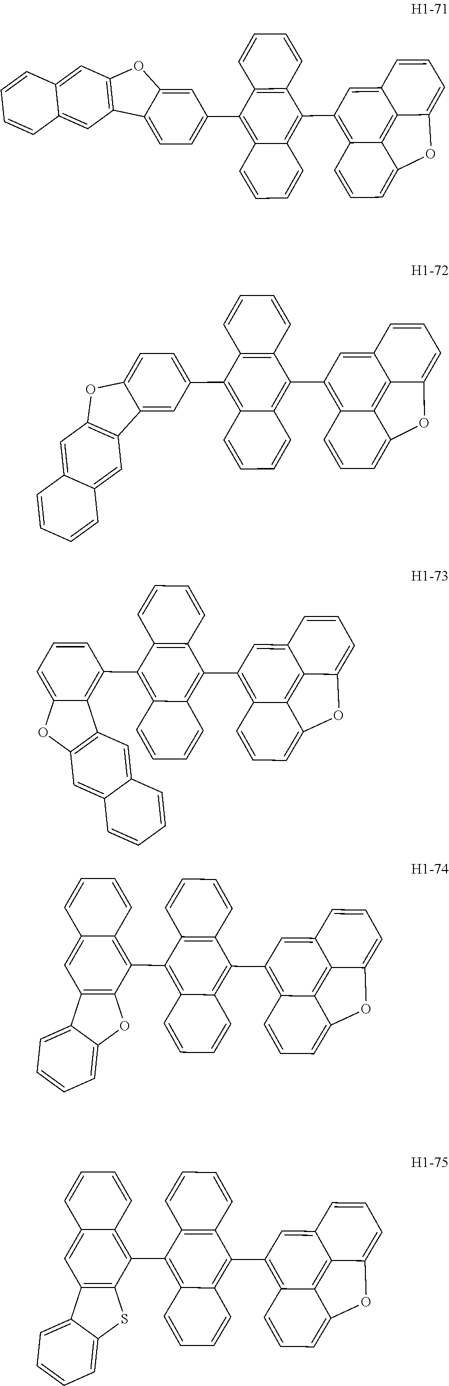

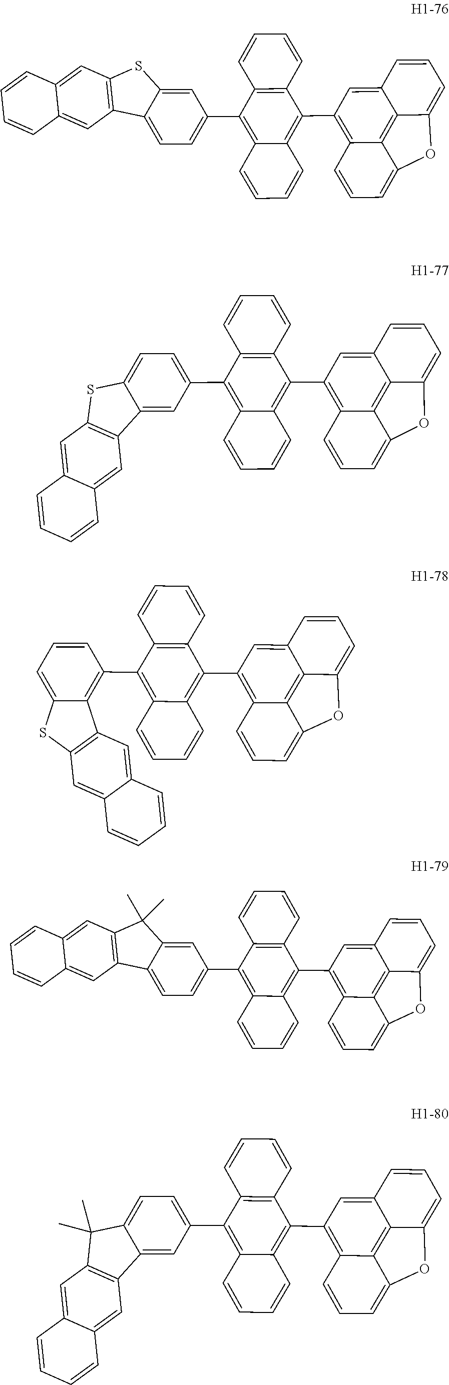 [SMILES: c1ccc2cc3c(cc2c1)sc1cc(-c2c4ccccc4c(-c4cc5cccc6oc7cccc4c7c56)c4ccccc24)ccc13 ; c1ccc2cc3c(cc2c1)sc1ccc(-c2c4ccccc4c(-c4cc5cccc6oc7cccc4c7c56)c4ccccc24)cc13 ; c1ccc2cc3c(cc2c1)sc1cccc(-c2c4ccccc4c(-c4cc5cccc6oc7cccc4c7c56)c4ccccc24)c13 ; CC1(C)c2cc(-c3c4ccccc4c(-c4cc5cccc6oc7cccc4c7c56)c4ccccc34)ccc2-c2cc3ccccc3cc21 ; CC1(C)c2ccc(-c3c4ccccc4c(-c4cc5cccc6oc7cccc4c7c56)c4ccccc34)cc2-c2cc3ccccc3cc21]

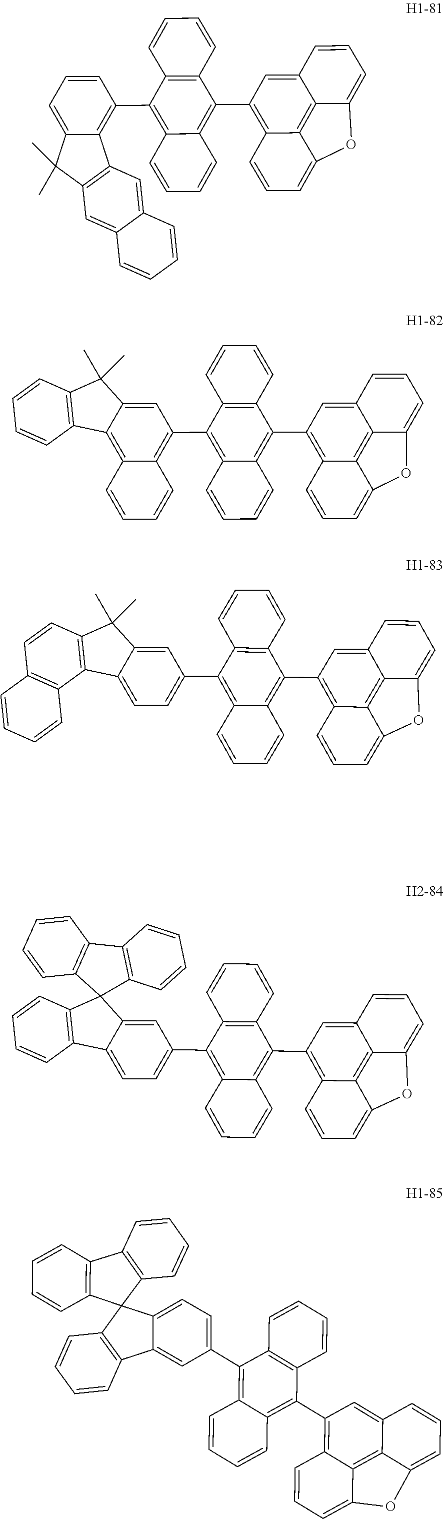 [SMILES: CC1(C)c2cc3ccccc3cc2-c2c(-c3c4ccccc4c(-c4cc5cccc6oc7cccc4c7c56)c4ccccc34)cccc21 ; CC1(C)c2ccccc2-c2c1cc(-c1c3ccccc3c(-c3cc4cccc5oc6cccc3c6c45)c3ccccc13)c1ccccc21 ; CC1(C)c2cc(-c3c4ccccc4c(-c4cc5cccc6oc7cccc4c7c56)c4ccccc34)ccc2-c2c1ccc1ccccc21 ; c1ccc2c(c1)-c1ccccc1C21c2ccccc2-c2ccc(-c3c4ccccc4c(-c4cc5cccc6oc7cccc4c7c56)c4ccccc34)cc21 ; c1ccc2c(c1)-c1ccccc1C21c2ccccc2-c2cc(-c3c4ccccc4c(-c4cc5cccc6oc7cccc4c7c56)c4ccccc34)ccc21]

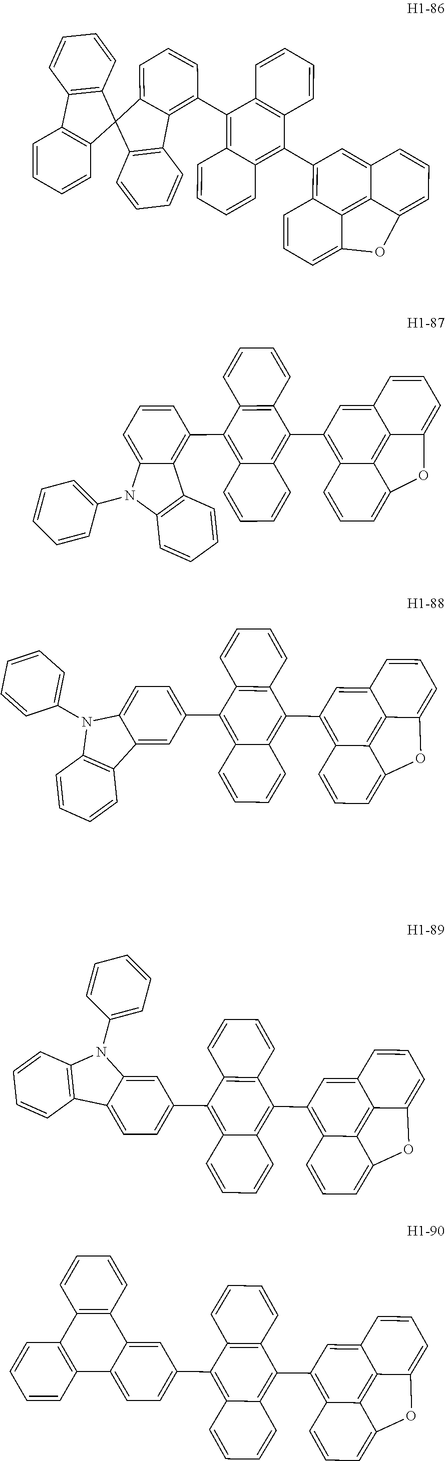 [SMILES: c1ccc2c(c1)-c1ccccc1C21c2ccccc2-c2c(-c3c4ccccc4c(-c4cc5cccc6oc7cccc4c7c56)c4ccccc34)cccc21 ; c1ccc(-n2c3ccccc3c3c(-c4c5ccccc5c(-c5cc6cccc7oc8cccc5c8c67)c5ccccc45)cccc32)cc1 ; c1ccc(-n2c3ccccc3c3cc(-c4c5ccccc5c(-c5cc6cccc7oc8cccc5c8c67)c5ccccc45)ccc32)cc1 ; c1ccc(-n2c3ccccc3c3ccc(-c4c5ccccc5c(-c5cc6cccc7oc8cccc5c8c67)c5ccccc45)cc32)cc1 ; c1cc2cc(-c3c4ccccc4c(-c4ccc5c6ccccc6c6ccccc6c5c4)c4ccccc34)c3cccc4oc(c1)c2c43]

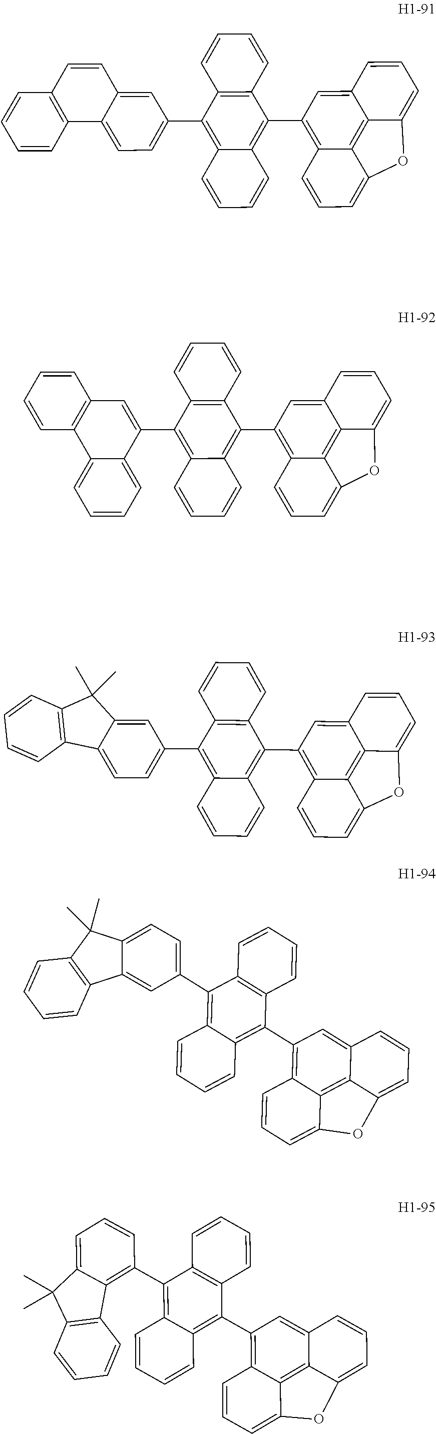 [SMILES: c1ccc2c(c1)ccc1cc(-c3c4ccccc4c(-c4cc5cccc6oc7cccc4c7c56)c4ccccc34)ccc12 ; c1ccc2c(c1)cc(-c1c3ccccc3c(-c3cc4cccc5oc6cccc3c6c45)c3ccccc13)c1ccccc12 ; CC1(C)c2ccccc2-c2ccc(-c3c4ccccc4c(-c4cc5cccc6oc7cccc4c7c56)c4ccccc34)cc21 ; CC1(C)c2ccccc2-c2cc(-c3c4ccccc4c(-c4cc5cccc6oc7cccc4c7c56)c4ccccc34)ccc21 ; CC1(C)c2ccccc2-c2c(-c3c4ccccc4c(-c4cc5cccc6oc7cccc4c7c56)c4ccccc34)cccc21]

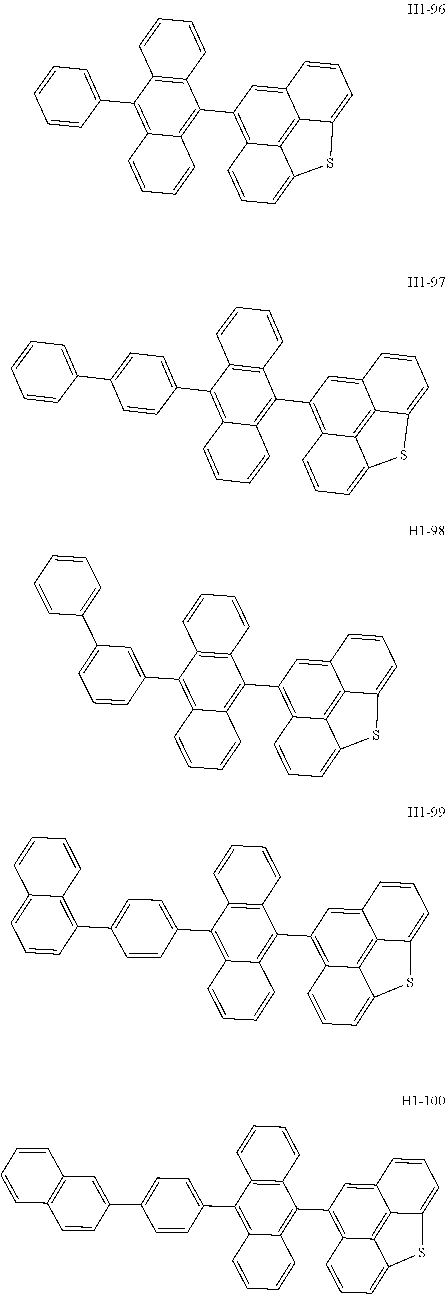 [SMILES: c1ccc(-c2c3ccccc3c(-c3cc4cccc5sc6cccc3c6c45)c3ccccc23)cc1 ; c1ccc(-c2ccc(-c3c4ccccc4c(-c4cc5cccc6sc7cccc4c7c56)c4ccccc34)cc2)cc1 ; c1ccc(-c2cccc(-c3c4ccccc4c(-c4cc5cccc6sc7cccc4c7c56)c4ccccc34)c2)cc1 ; c1ccc2c(-c3ccc(-c4c5ccccc5c(-c5cc6cccc7sc8cccc5c8c67)c5ccccc45)cc3)cccc2c1 ; c1ccc2cc(-c3ccc(-c4c5ccccc5c(-c5cc6cccc7sc8cccc5c8c67)c5ccccc45)cc3)ccc2c1]

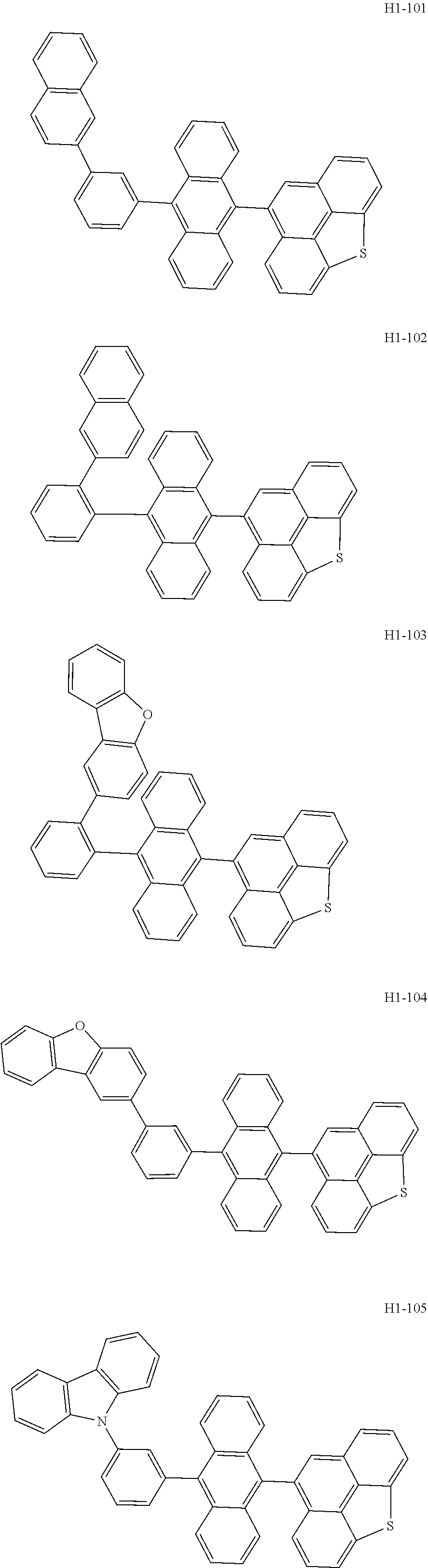 [SMILES: c1cc(-c2ccc3ccccc3c2)cc(-c2c3ccccc3c(-c3cc4cccc5sc6cccc3c6c45)c3ccccc23)c1 ; c1ccc(-c2c3ccccc3c(-c3cc4cccc5sc6cccc3c6c45)c3ccccc23)c(-c2ccc3ccccc3c2)c1 ; c1ccc(-c2c3ccccc3c(-c3cc4cccc5sc6cccc3c6c45)c3ccccc23)c(-c2ccc3oc4ccccc4c3c2)c1 ; c1cc(-c2ccc3oc4ccccc4c3c2)cc(-c2c3ccccc3c(-c3cc4cccc5sc6cccc3c6c45)c3ccccc23)c1 ; c1cc(-c2c3ccccc3c(-c3cc4cccc5sc6cccc3c6c45)c3ccccc23)cc(-n2c3ccccc3c3ccccc32)c1]

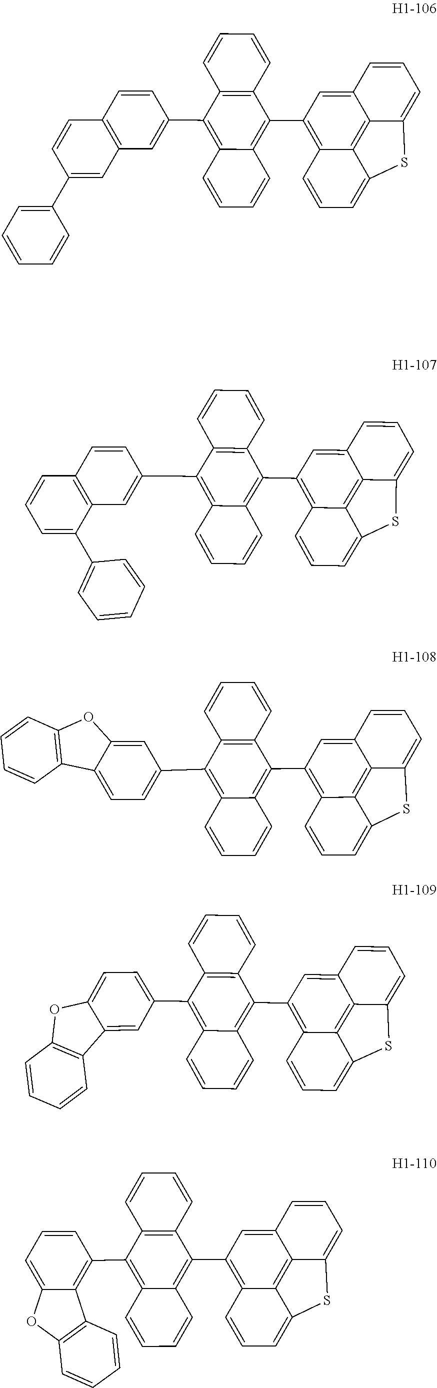 [SMILES: c1ccc(-c2ccc3ccc(-c4c5ccccc5c(-c5cc6cccc7sc8cccc5c8c67)c5ccccc45)cc3c2)cc1 ; c1ccc(-c2cccc3ccc(-c4c5ccccc5c(-c5cc6cccc7sc8cccc5c8c67)c5ccccc45)cc23)cc1 ; c1ccc2c(c1)oc1cc(-c3c4ccccc4c(-c4cc5cccc6sc7cccc4c7c56)c4ccccc34)ccc12 ; c1ccc2c(c1)oc1ccc(-c3c4ccccc4c(-c4cc5cccc6sc7cccc4c7c56)c4ccccc34)cc12 ; c1ccc2c(c1)oc1cccc(-c3c4ccccc4c(-c4cc5cccc6sc7cccc4c7c56)c4ccccc34)c12]

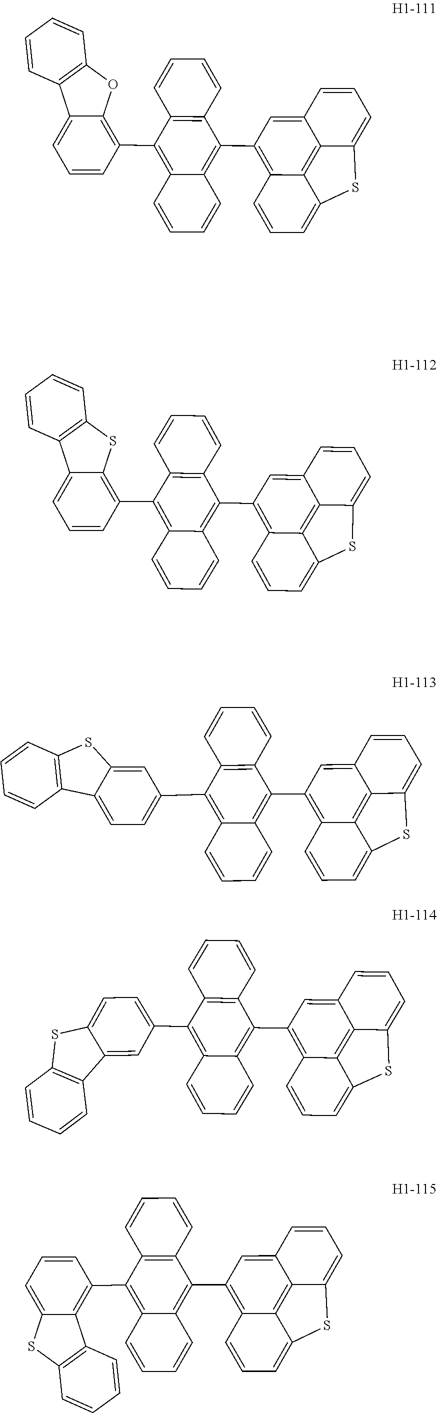 [SMILES: c1ccc2c(c1)oc1c(-c3c4ccccc4c(-c4cc5cccc6sc7cccc4c7c56)c4ccccc34)cccc12 ; c1ccc2c(c1)sc1c(-c3c4ccccc4c(-c4cc5cccc6sc7cccc4c7c56)c4ccccc34)cccc12 ; c1ccc2c(c1)sc1cc(-c3c4ccccc4c(-c4cc5cccc6sc7cccc4c7c56)c4ccccc34)ccc12 ; c1ccc2c(c1)sc1ccc(-c3c4ccccc4c(-c4cc5cccc6sc7cccc4c7c56)c4ccccc34)cc12 ; c1ccc2c(c1)sc1cccc(-c3c4ccccc4c(-c4cc5cccc6sc7cccc4c7c56)c4ccccc34)c12]

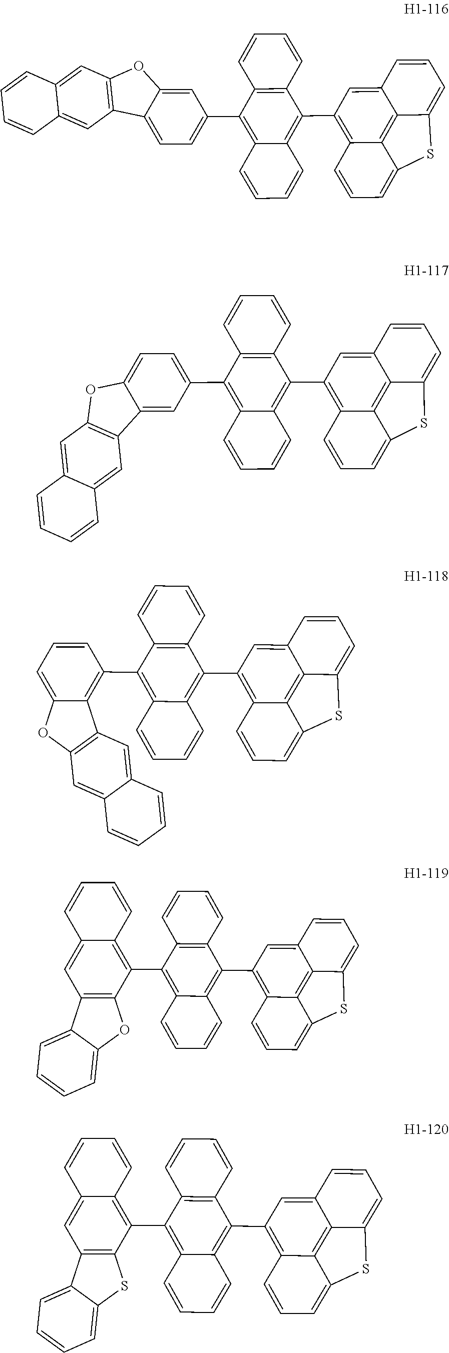

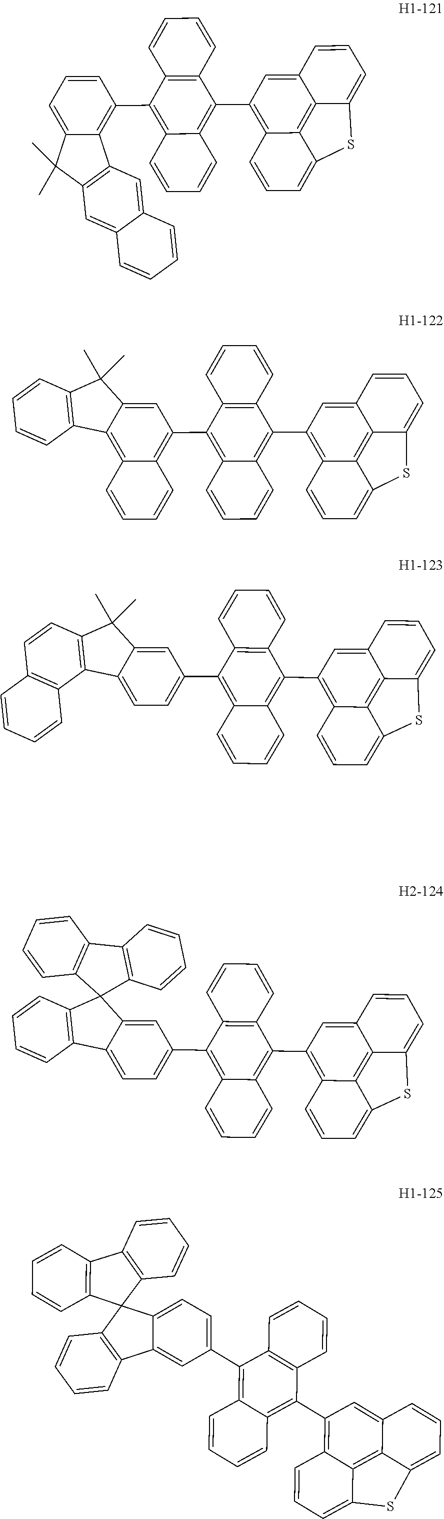 [SMILES: CC1(C)c2cc3ccccc3cc2-c2c(-c3c4ccccc4c(-c4cc5cccc6sc7cccc4c7c56)c4ccccc34)cccc21 ; CC1(C)c2ccccc2-c2c1cc(-c1c3ccccc3c(-c3cc4cccc5sc6cccc3c6c45)c3ccccc13)c1ccccc21 ; CC1(C)c2cc(-c3c4ccccc4c(-c4cc5cccc6sc7cccc4c7c56)c4ccccc34)ccc2-c2c1ccc1ccccc21 ; c1ccc2c(c1)-c1ccccc1C21c2ccccc2-c2ccc(-c3c4ccccc4c(-c4cc5cccc6sc7cccc4c7c56)c4ccccc34)cc21 ; c1ccc2c(c1)-c1ccccc1C21c2ccccc2-c2cc(-c3c4ccccc4c(-c4cc5cccc6sc7cccc4c7c56)c4ccccc34)ccc21]

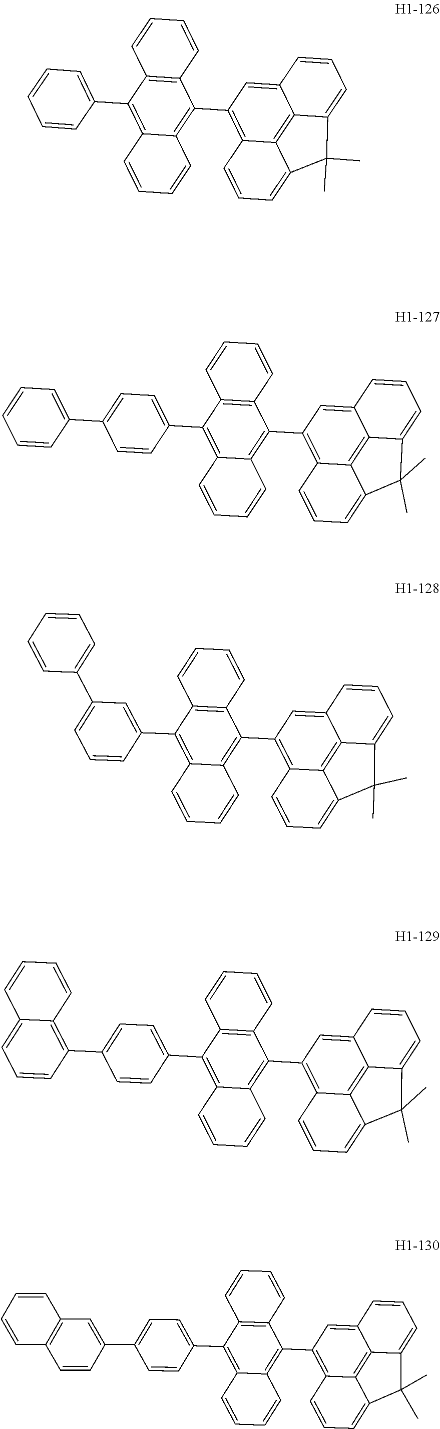

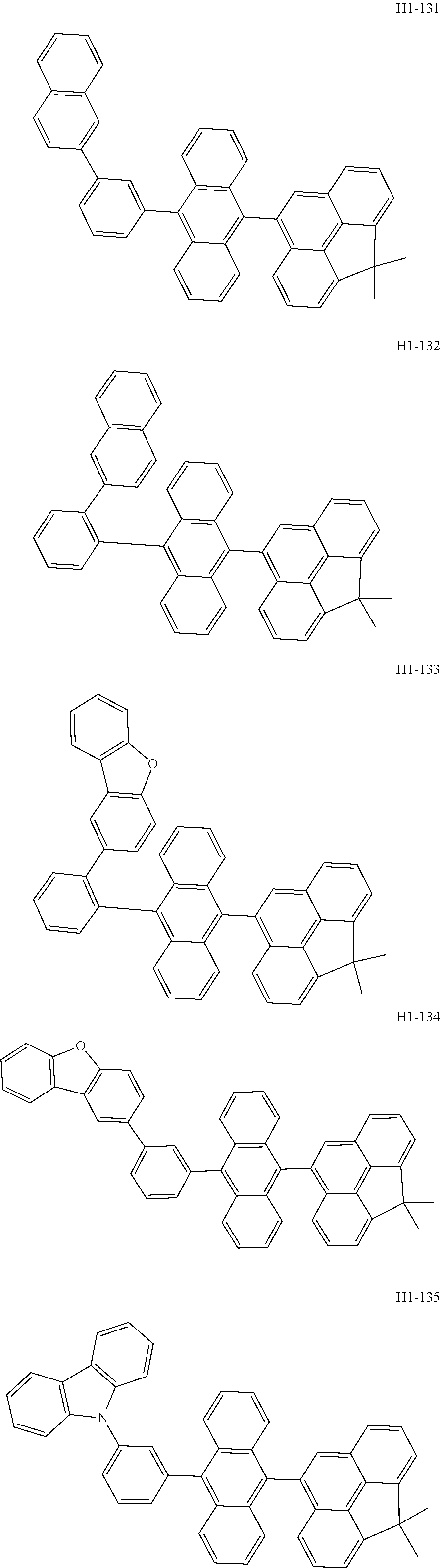

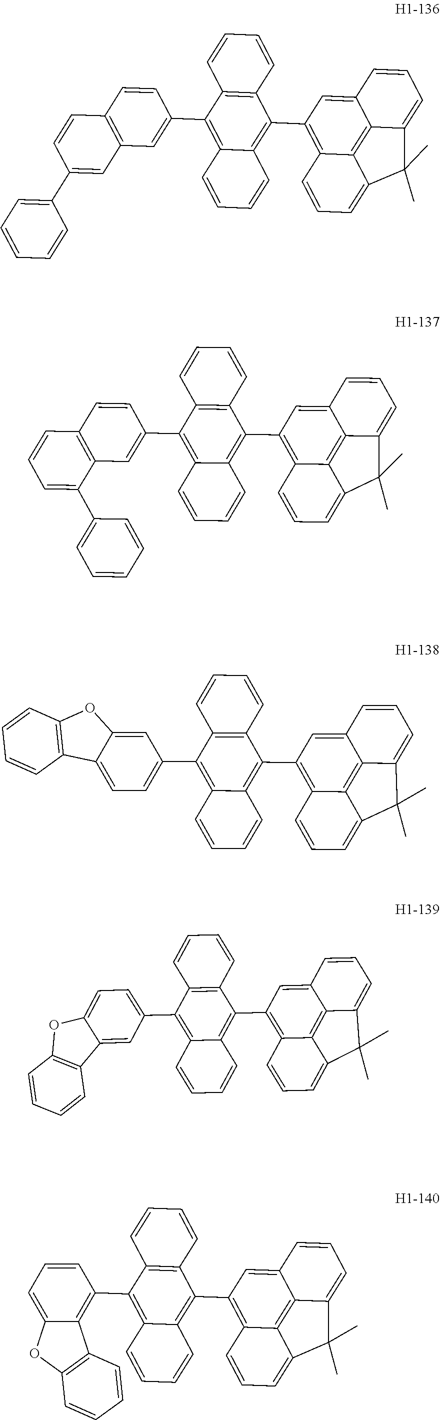

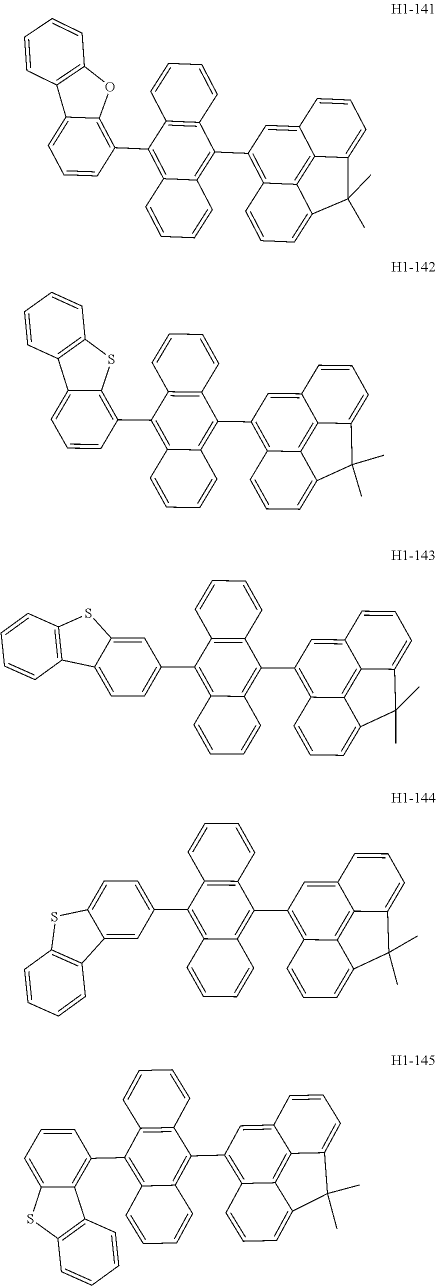 [SMILES: CC1(C)c2cccc3cc(-c4c5ccccc5c(-c5cccc6c5oc5ccccc56)c5ccccc45)c4cccc1c4c23 ; CC1(C)c2cccc3cc(-c4c5ccccc5c(-c5cccc6c5sc5ccccc56)c5ccccc45)c4cccc1c4c23 ; CC1(C)c2cccc3cc(-c4c5ccccc5c(-c5ccc6c(c5)sc5ccccc56)c5ccccc45)c4cccc1c4c23 ; CC1(C)c2cccc3cc(-c4c5ccccc5c(-c5ccc6sc7ccccc7c6c5)c5ccccc45)c4cccc1c4c23 ; CC1(C)c2cccc3cc(-c4c5ccccc5c(-c5cccc6sc7ccccc7c56)c5ccccc45)c4cccc1c4c23]

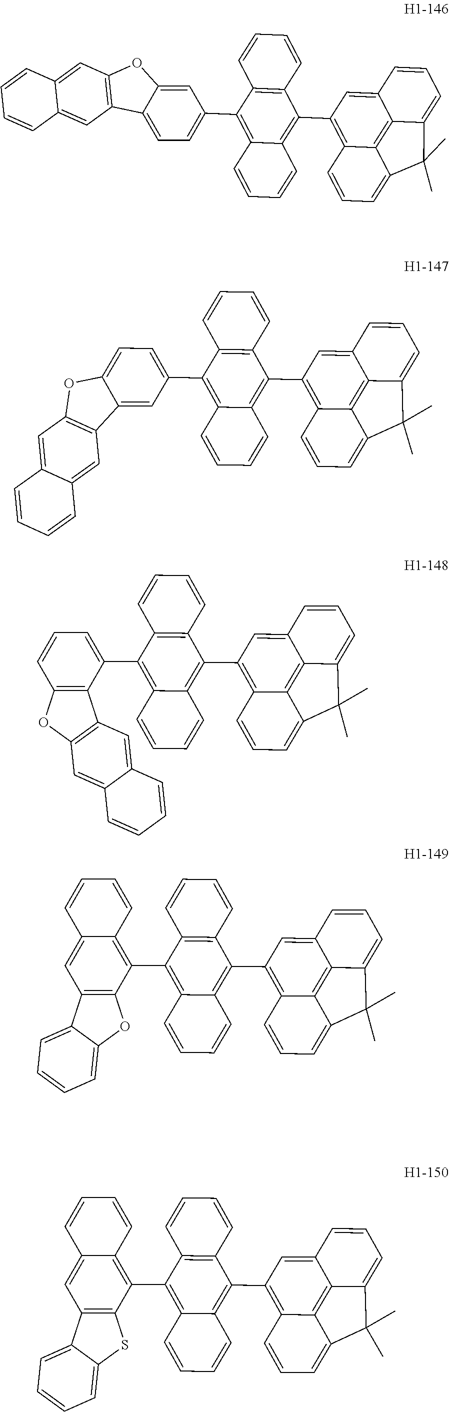 [SMILES: CC1(C)c2cccc3cc(-c4c5ccccc5c(-c5ccc6c(c5)oc5cc7ccccc7cc56)c5ccccc45)c4cccc1c4c23 ; CC1(C)c2cccc3cc(-c4c5ccccc5c(-c5ccc6oc7cc8ccccc8cc7c6c5)c5ccccc45)c4cccc1c4c23 ; CC1(C)c2cccc3cc(-c4c5ccccc5c(-c5cccc6oc7cc8ccccc8cc7c56)c5ccccc45)c4cccc1c4c23 ; CC1(C)c2cccc3cc(-c4c5ccccc5c(-c5c6ccccc6cc6c5oc5ccccc56)c5ccccc45)c4cccc1c4c23 ; CC1(C)c2cccc3cc(-c4c5ccccc5c(-c5c6ccccc6cc6c5sc5ccccc56)c5ccccc45)c4cccc1c4c23]

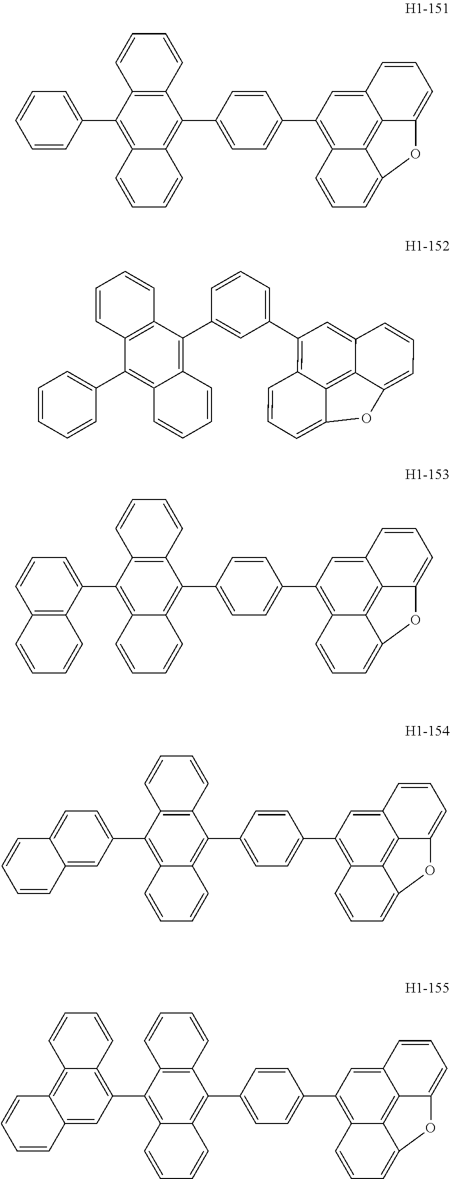 [SMILES: c1ccc(-c2c3ccccc3c(-c3ccc(-c4cc5cccc6oc7cccc4c7c56)cc3)c3ccccc23)cc1 ; c1ccc(-c2c3ccccc3c(-c3cccc(-c4cc5cccc6oc7cccc4c7c56)c3)c3ccccc23)cc1 ; c1ccc2c(-c3c4ccccc4c(-c4ccc(-c5cc6cccc7oc8cccc5c8c67)cc4)c4ccccc34)cccc2c1 ; c1ccc2cc(-c3c4ccccc4c(-c4ccc(-c5cc6cccc7oc8cccc5c8c67)cc4)c4ccccc34)ccc2c1 ; c1ccc2c(c1)cc(-c1c3ccccc3c(-c3ccc(-c4cc5cccc6oc7cccc4c7c56)cc3)c3ccccc13)c1ccccc12]

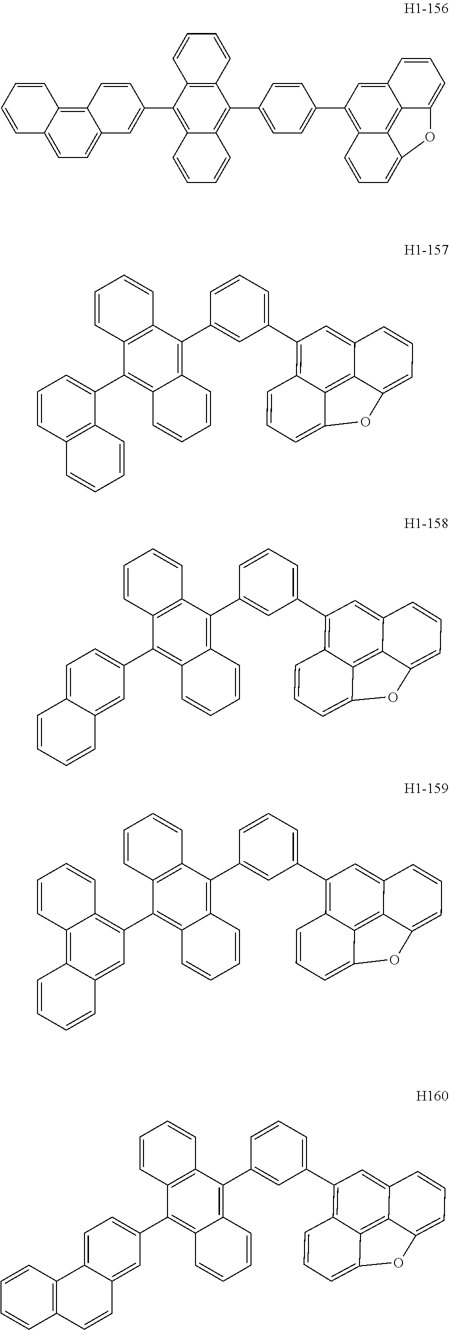 [SMILES: c1ccc2c(c1)ccc1cc(-c3c4ccccc4c(-c4ccc(-c5cc6cccc7oc8cccc5c8c67)cc4)c4ccccc34)ccc12 ; c1cc(-c2c3ccccc3c(-c3cccc4ccccc34)c3ccccc23)cc(-c2cc3cccc4oc5cccc2c5c34)c1 ; c1cc(-c2c3ccccc3c(-c3ccc4ccccc4c3)c3ccccc23)cc(-c2cc3cccc4oc5cccc2c5c34)c1 ; c1cc(-c2c3ccccc3c(-c3cc4ccccc4c4ccccc34)c3ccccc23)cc(-c2cc3cccc4oc5cccc2c5c34)c1 ; c1cc(-c2c3ccccc3c(-c3ccc4c(ccc5ccccc54)c3)c3ccccc23)cc(-c2cc3cccc4oc5cccc2c5c34)c1]

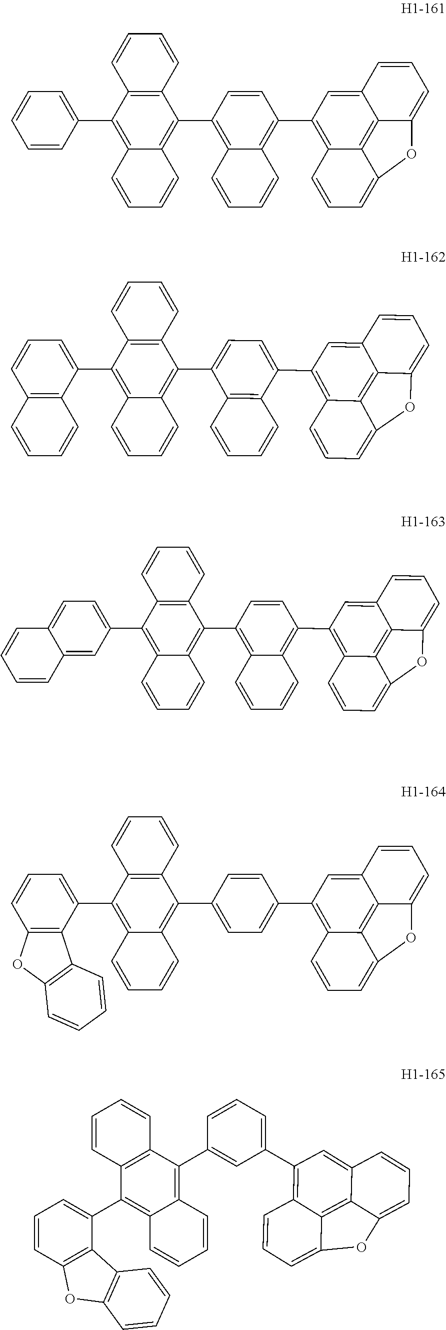 [SMILES: c1ccc(-c2c3ccccc3c(-c3ccc(-c4cc5cccc6oc7cccc4c7c56)c4ccccc34)c3ccccc23)cc1 ; c1ccc2c(-c3c4ccccc4c(-c4ccc(-c5cc6cccc7oc8cccc5c8c67)c5ccccc45)c4ccccc34)cccc2c1 ; c1ccc2cc(-c3c4ccccc4c(-c4ccc(-c5cc6cccc7oc8cccc5c8c67)c5ccccc45)c4ccccc34)ccc2c1 ; c1ccc2c(c1)oc1cccc(-c3c4ccccc4c(-c4ccc(-c5cc6cccc7oc8cccc5c8c67)cc4)c4ccccc34)c12 ; c1cc(-c2c3ccccc3c(-c3cccc4oc5ccccc5c34)c3ccccc23)cc(-c2cc3cccc4oc5cccc2c5c34)c1]

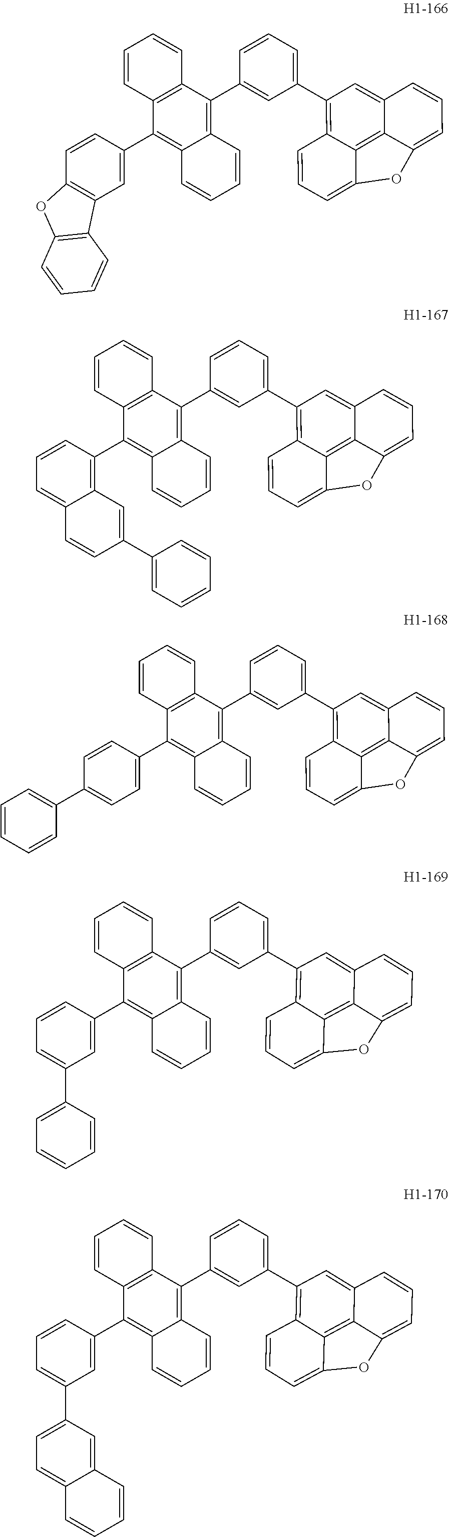

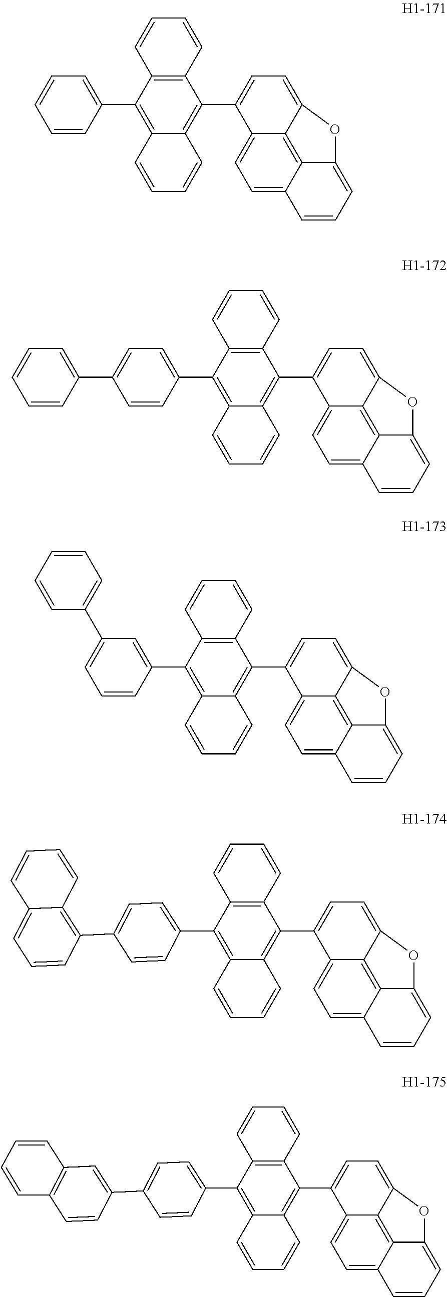 [SMILES: c1ccc(-c2c3ccccc3c(-c3ccc4oc5cccc6ccc3c4c65)c3ccccc23)cc1 ; c1ccc(-c2ccc(-c3c4ccccc4c(-c4ccc5oc6cccc7ccc4c5c76)c4ccccc34)cc2)cc1 ; c1ccc(-c2cccc(-c3c4ccccc4c(-c4ccc5oc6cccc7ccc4c5c76)c4ccccc34)c2)cc1 ; c1ccc2c(-c3ccc(-c4c5ccccc5c(-c5ccc6oc7cccc8ccc5c6c87)c5ccccc45)cc3)cccc2c1 ; c1ccc2cc(-c3ccc(-c4c5ccccc5c(-c5ccc6oc7cccc8ccc5c6c87)c5ccccc45)cc3)ccc2c1]

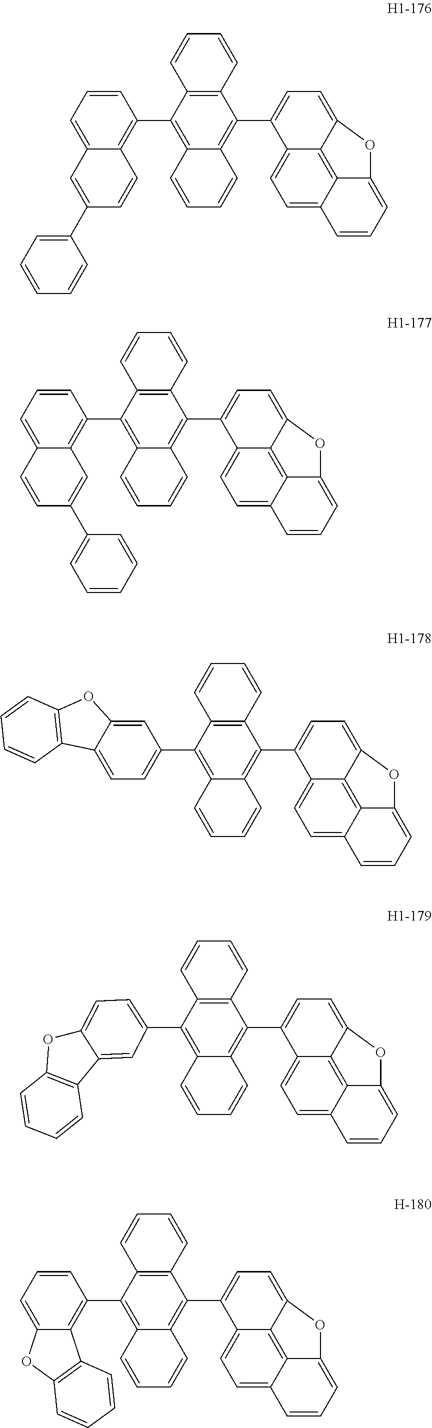 [SMILES: c1ccc(-c2ccc3c(-c4c5ccccc5c(-c5ccc6oc7cccc8ccc5c6c87)c5ccccc45)cccc3c2)cc1 ; c1ccc(-c2ccc3cccc(-c4c5ccccc5c(-c5ccc6oc7cccc8ccc5c6c87)c5ccccc45)c3c2)cc1 ; c1ccc2c(c1)oc1cc(-c3c4ccccc4c(-c4ccc5oc6cccc7ccc4c5c76)c4ccccc34)ccc12 ; c1ccc2c(c1)oc1ccc(-c3c4ccccc4c(-c4ccc5oc6cccc7ccc4c5c76)c4ccccc34)cc12 ; c1ccc2c(c1)oc1cccc(-c3c4ccccc4c(-c4ccc5oc6cccc7ccc4c5c76)c4ccccc34)c12]

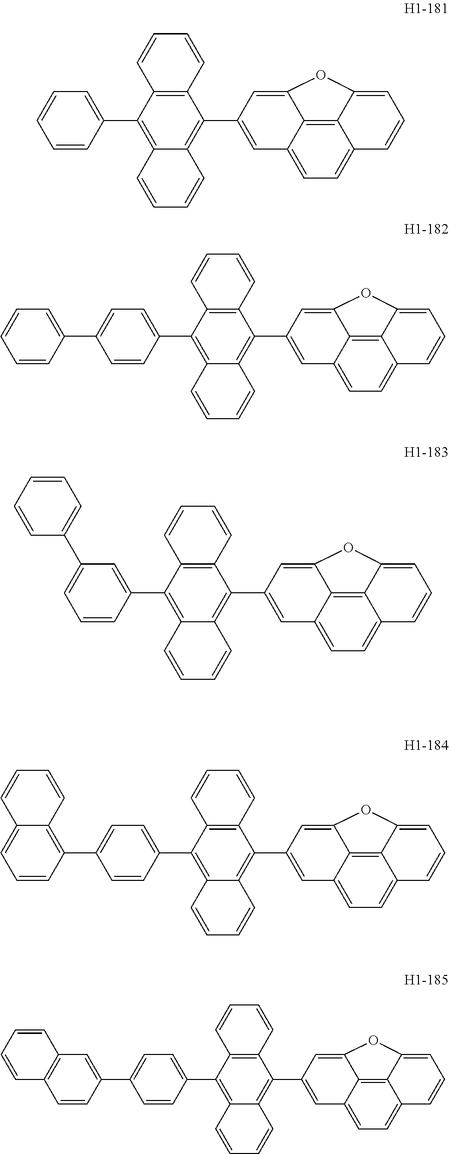 [SMILES: c1ccc(-c2c3ccccc3c(-c3cc4ccc5cccc6oc(c3)c4c56)c3ccccc23)cc1 ; c1ccc(-c2ccc(-c3c4ccccc4c(-c4cc5ccc6cccc7oc(c4)c5c67)c4ccccc34)cc2)cc1 ; c1ccc(-c2cccc(-c3c4ccccc4c(-c4cc5ccc6cccc7oc(c4)c5c67)c4ccccc34)c2)cc1 ; c1ccc2c(-c3ccc(-c4c5ccccc5c(-c5cc6ccc7cccc8oc(c5)c6c78)c5ccccc45)cc3)cccc2c1 ; c1ccc2cc(-c3ccc(-c4c5ccccc5c(-c5cc6ccc7cccc8oc(c5)c6c78)c5ccccc45)cc3)ccc2c1]

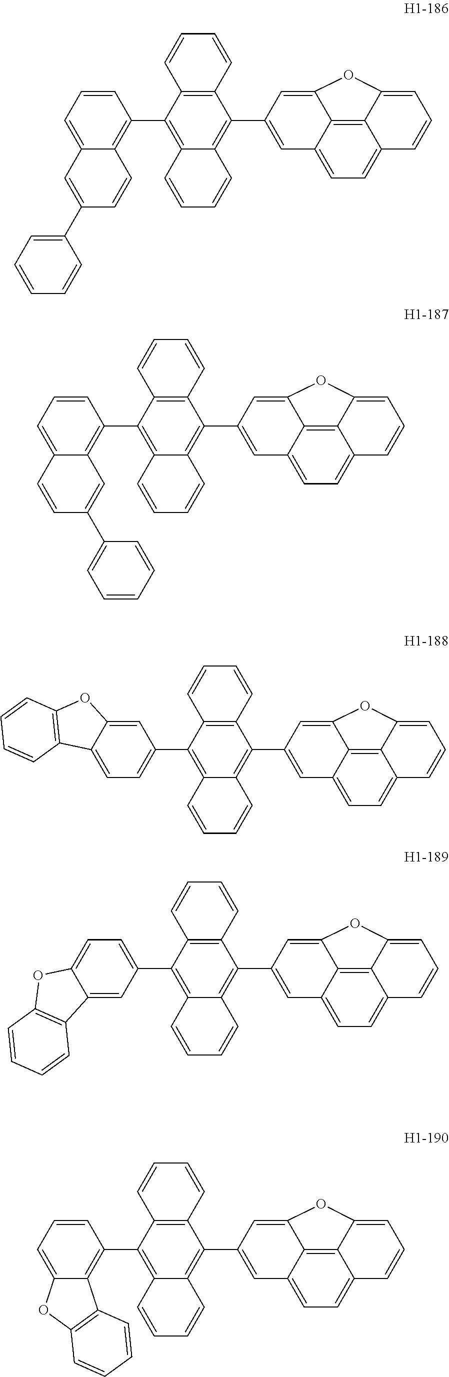 [SMILES: c1ccc(-c2ccc3c(-c4c5ccccc5c(-c5cc6ccc7cccc8oc(c5)c6c78)c5ccccc45)cccc3c2)cc1 ; c1ccc(-c2ccc3cccc(-c4c5ccccc5c(-c5cc6ccc7cccc8oc(c5)c6c78)c5ccccc45)c3c2)cc1 ; c1ccc2c(c1)oc1cc(-c3c4ccccc4c(-c4cc5ccc6cccc7oc(c4)c5c67)c4ccccc34)ccc12 ; c1ccc2c(c1)oc1ccc(-c3c4ccccc4c(-c4cc5ccc6cccc7oc(c4)c5c67)c4ccccc34)cc12 ; c1ccc2c(c1)oc1cccc(-c3c4ccccc4c(-c4cc5ccc6cccc7oc(c4)c5c67)c4ccccc34)c12]

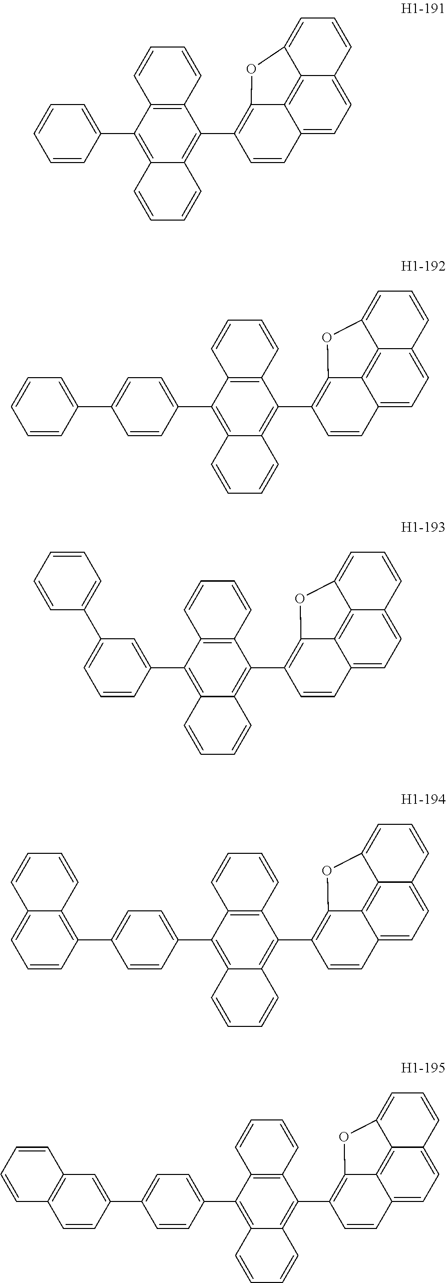 [SMILES: c1ccc(-c2c3ccccc3c(-c3ccc4ccc5cccc6oc3c4c56)c3ccccc23)cc1 ; c1ccc(-c2ccc(-c3c4ccccc4c(-c4ccc5ccc6cccc7oc4c5c67)c4ccccc34)cc2)cc1 ; c1ccc(-c2cccc(-c3c4ccccc4c(-c4ccc5ccc6cccc7oc4c5c67)c4ccccc34)c2)cc1 ; c1ccc2c(-c3ccc(-c4c5ccccc5c(-c5ccc6ccc7cccc8oc5c6c78)c5ccccc45)cc3)cccc2c1 ; c1ccc2cc(-c3ccc(-c4c5ccccc5c(-c5ccc6ccc7cccc8oc5c6c78)c5ccccc45)cc3)ccc2c1]

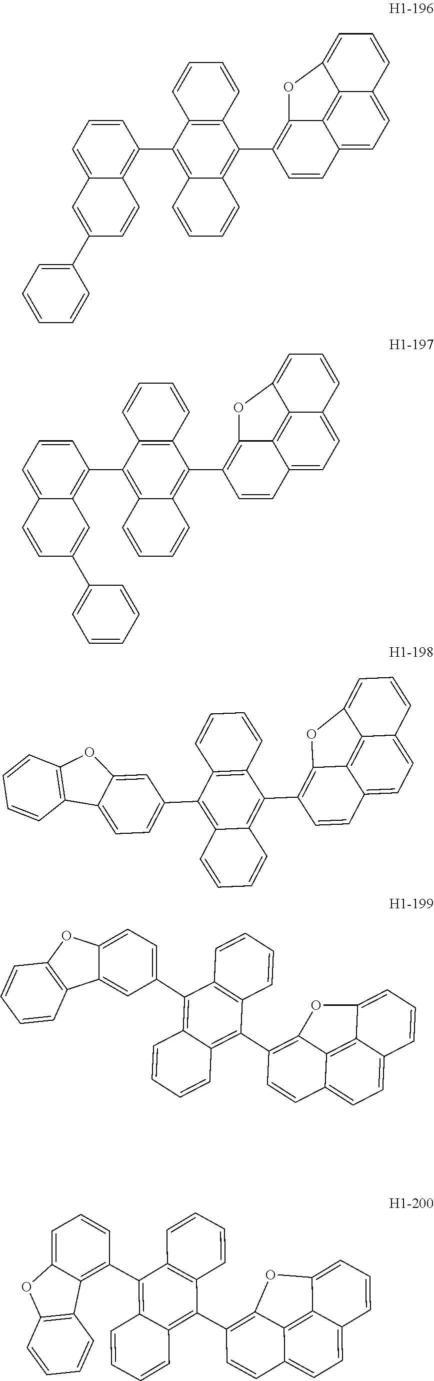 [SMILES: c1ccc(-c2ccc3c(-c4c5ccccc5c(-c5ccc6ccc7cccc8oc5c6c78)c5ccccc45)cccc3c2)cc1 ; c1ccc(-c2ccc3cccc(-c4c5ccccc5c(-c5ccc6ccc7cccc8oc5c6c78)c5ccccc45)c3c2)cc1 ; c1ccc2c(c1)oc1cc(-c3c4ccccc4c(-c4ccc5ccc6cccc7oc4c5c67)c4ccccc34)ccc12 ; c1ccc2c(c1)oc1ccc(-c3c4ccccc4c(-c4ccc5ccc6cccc7oc4c5c67)c4ccccc34)cc12 ; c1ccc2c(c1)oc1cccc(-c3c4ccccc4c(-c4ccc5ccc6cccc7oc4c5c67)c4ccccc34)c12]

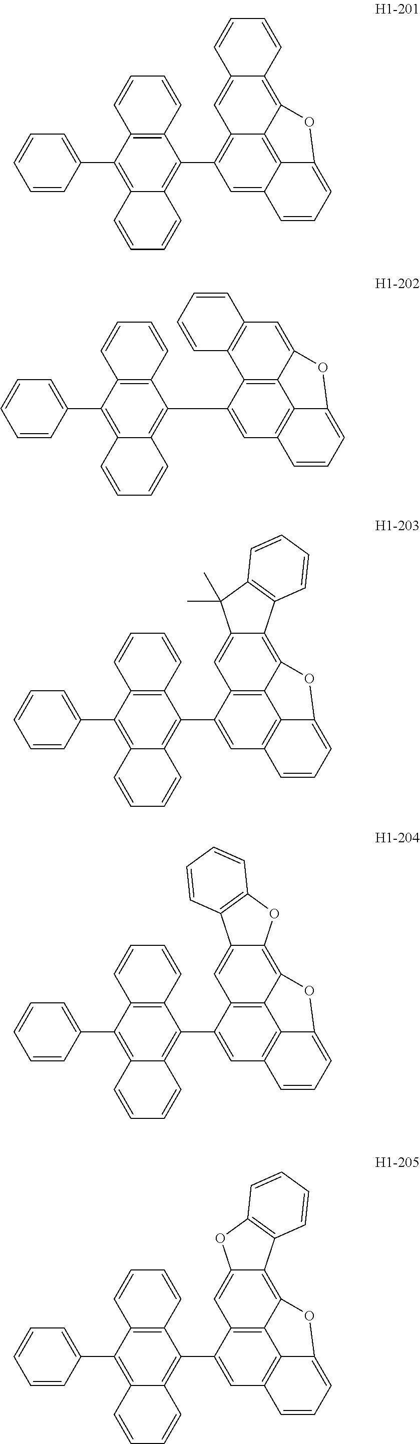 [SMILES: c1ccc(-c2c3ccccc3c(-c3cc4cccc5oc6c7ccccc7cc3c6c45)c3ccccc23)cc1 ; c1ccc(-c2c3ccccc3c(-c3cc4cccc5oc6cc7ccccc7c3c6c45)c3ccccc23)cc1 ; CC1(C)c2ccccc2-c2c1cc1c(-c3c4ccccc4c(-c4ccccc4)c4ccccc34)cc3cccc4oc2c1c34 ; c1ccc(-c2c3ccccc3c(-c3cc4cccc5oc6c7oc8ccccc8c7cc3c6c45)c3ccccc23)cc1 ; c1ccc(-c2c3ccccc3c(-c3cc4cccc5oc6c7c(cc3c6c45)oc3ccccc37)c3ccccc23)cc1]

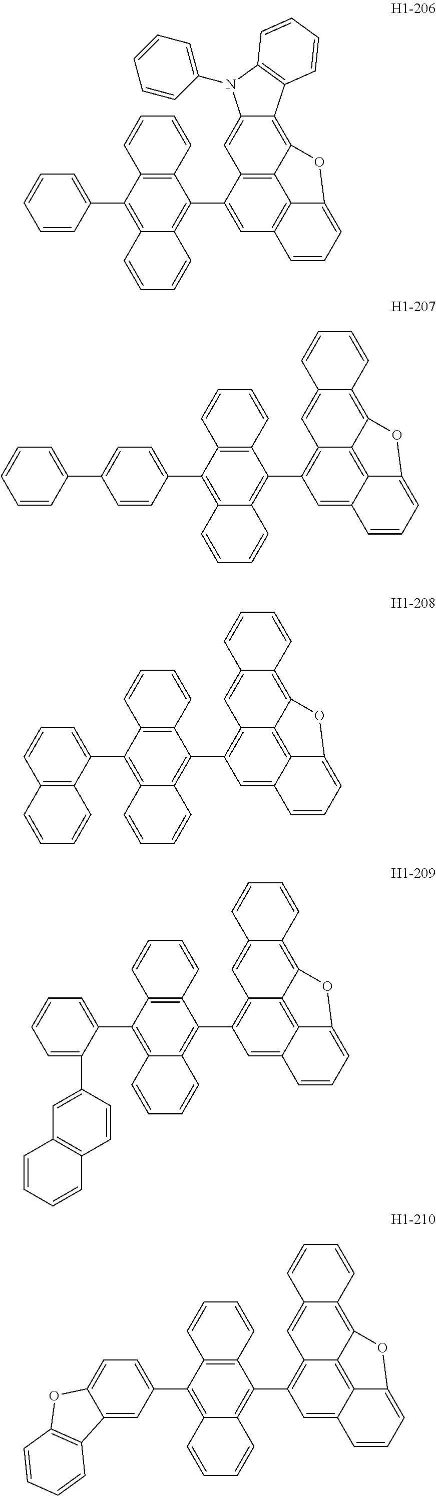 [SMILES: c1ccc(-c2c3ccccc3c(-c3cc4cccc5oc6c(c3cc3c6c6ccccc6n3-c3ccccc3)c45)c3ccccc23)cc1 ; c1ccc(-c2ccc(-c3c4ccccc4c(-c4cc5cccc6oc7c8ccccc8cc4c7c56)c4ccccc34)cc2)cc1 ; c1ccc2c(-c3c4ccccc4c(-c4cc5cccc6oc7c8ccccc8cc4c7c56)c4ccccc34)cccc2c1 ; c1ccc(-c2c3ccccc3c(-c3cc4cccc5oc6c7ccccc7cc3c6c45)c3ccccc23)c(-c2ccc3ccccc3c2)c1 ; c1ccc2c(c1)cc1c(-c3c4ccccc4c(-c4ccc5oc6ccccc6c5c4)c4ccccc34)cc3cccc4oc2c1c34]

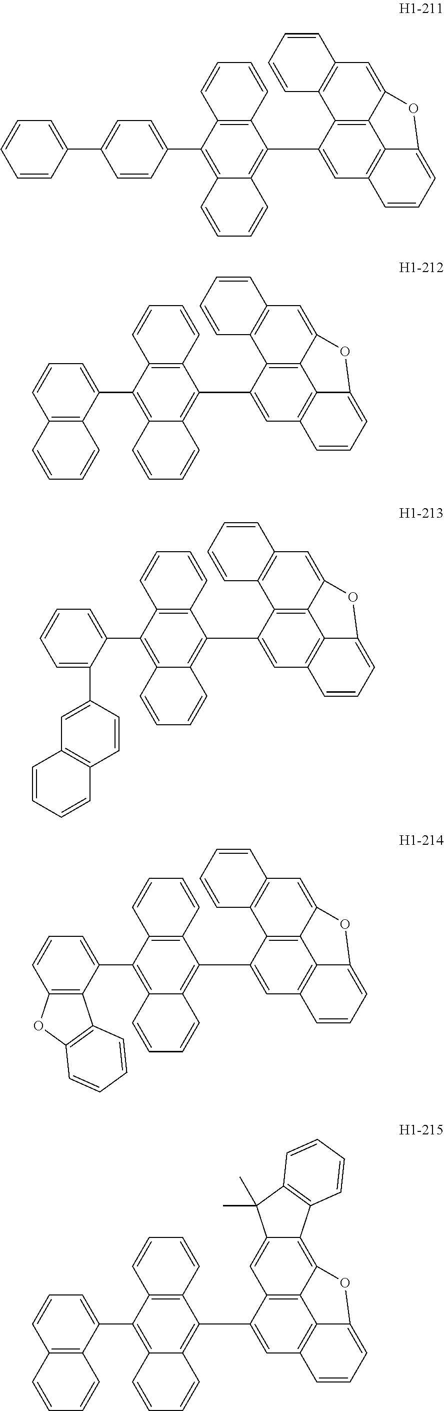

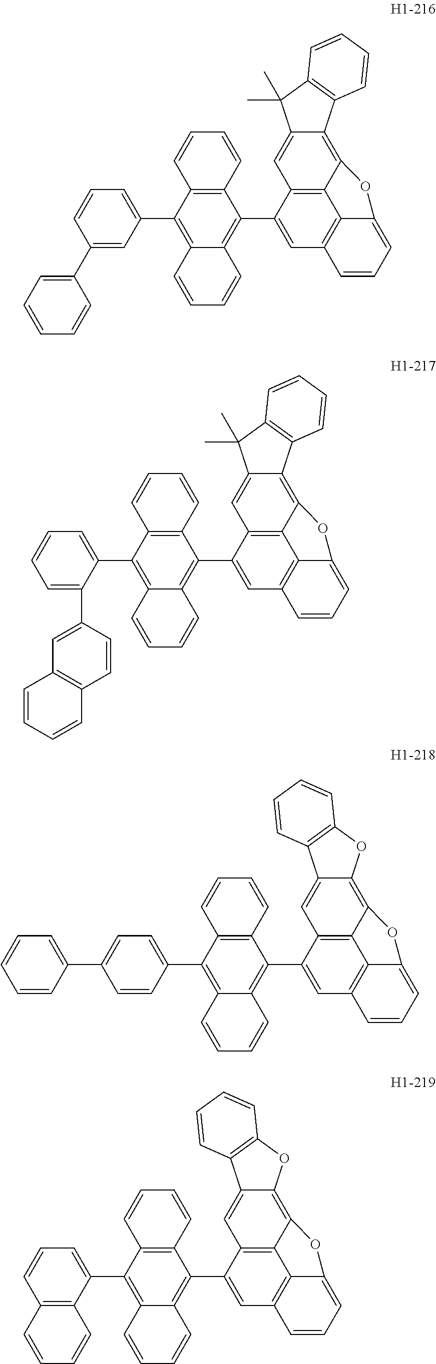

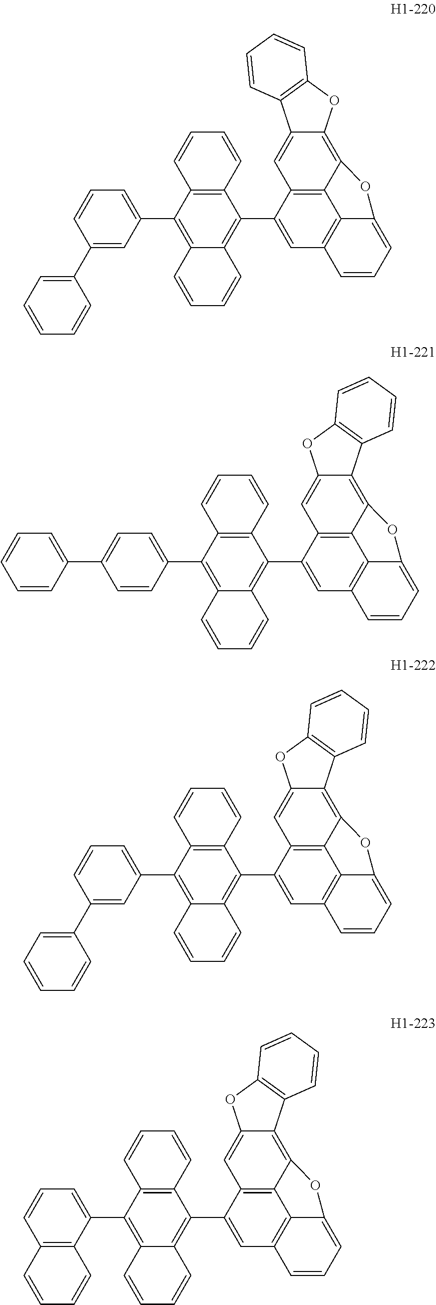 [SMILES: c1ccc(-c2cccc(-c3c4ccccc4c(-c4cc5cccc6oc7c8oc9ccccc9c8cc4c7c56)c4ccccc34)c2)cc1 ; c1ccc(-c2ccc(-c3c4ccccc4c(-c4cc5cccc6oc7c8c(cc4c7c56)oc4ccccc48)c4ccccc34)cc2)cc1 ; c1ccc(-c2cccc(-c3c4ccccc4c(-c4cc5cccc6oc7c8c(cc4c7c56)oc4ccccc48)c4ccccc34)c2)cc1 ; c1ccc2c(-c3c4ccccc4c(-c4cc5cccc6oc7c8c(cc4c7c56)oc4ccccc48)c4ccccc34)cccc2c1]

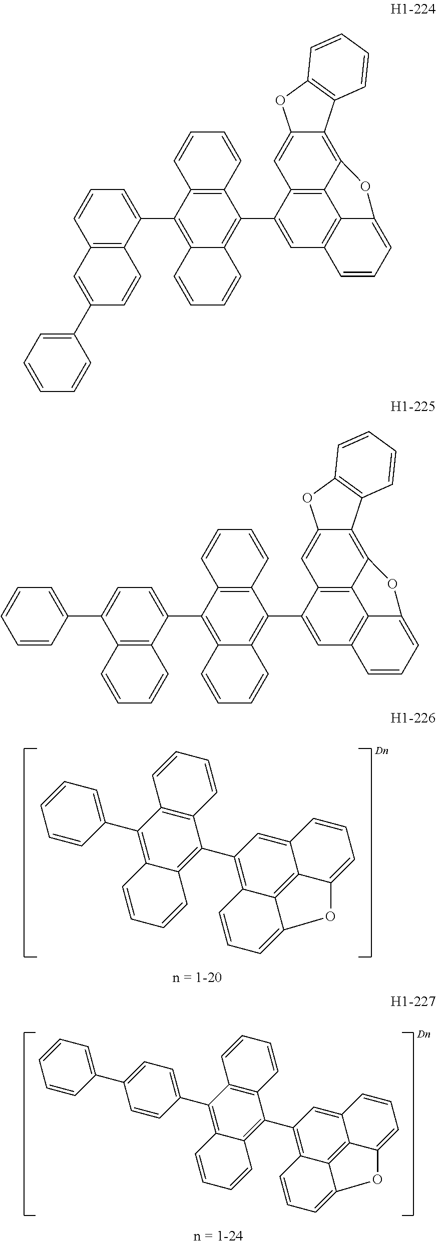

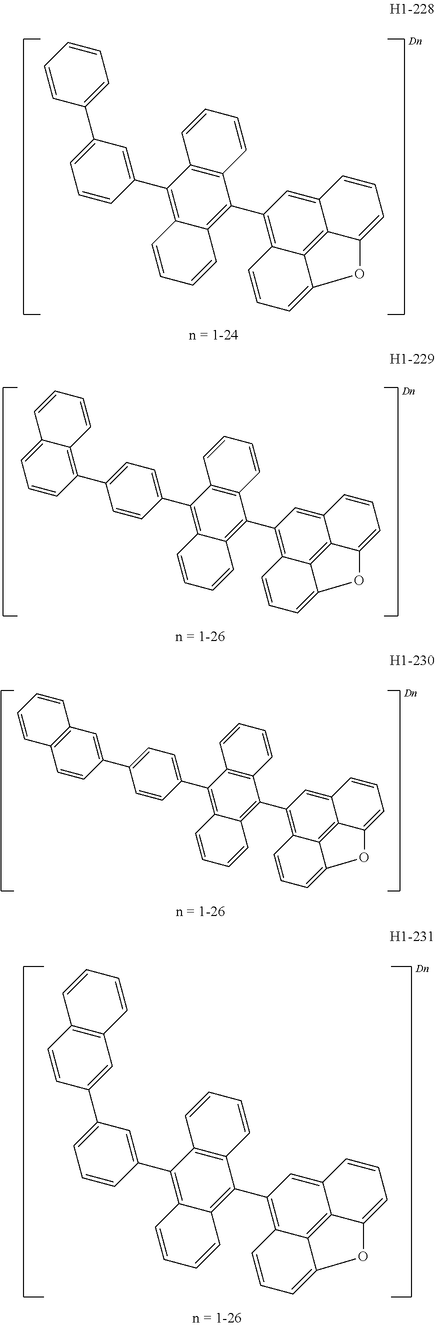

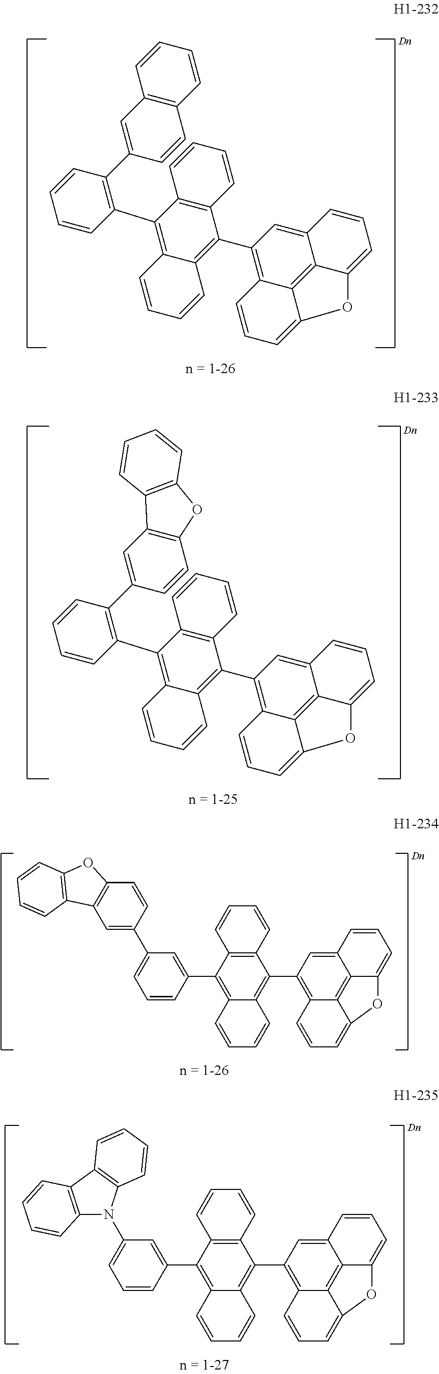

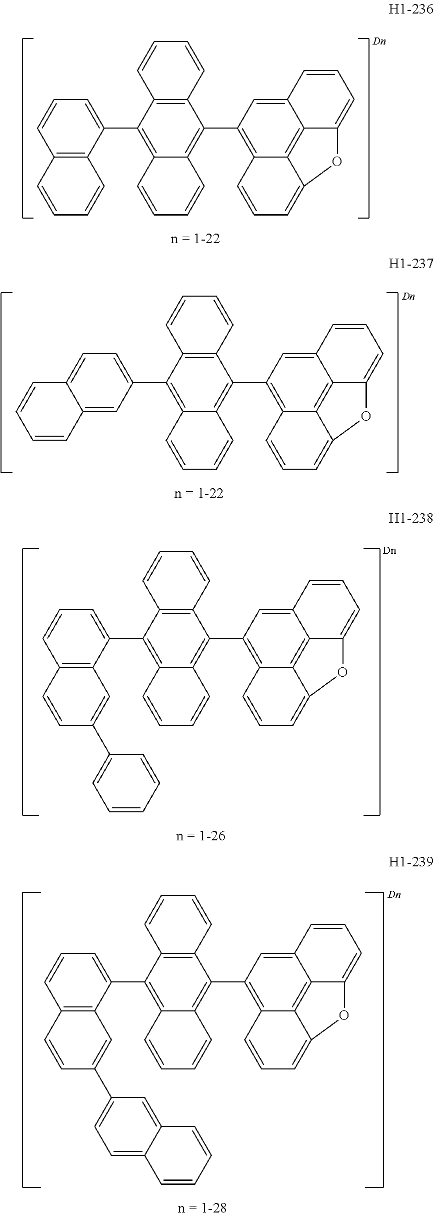

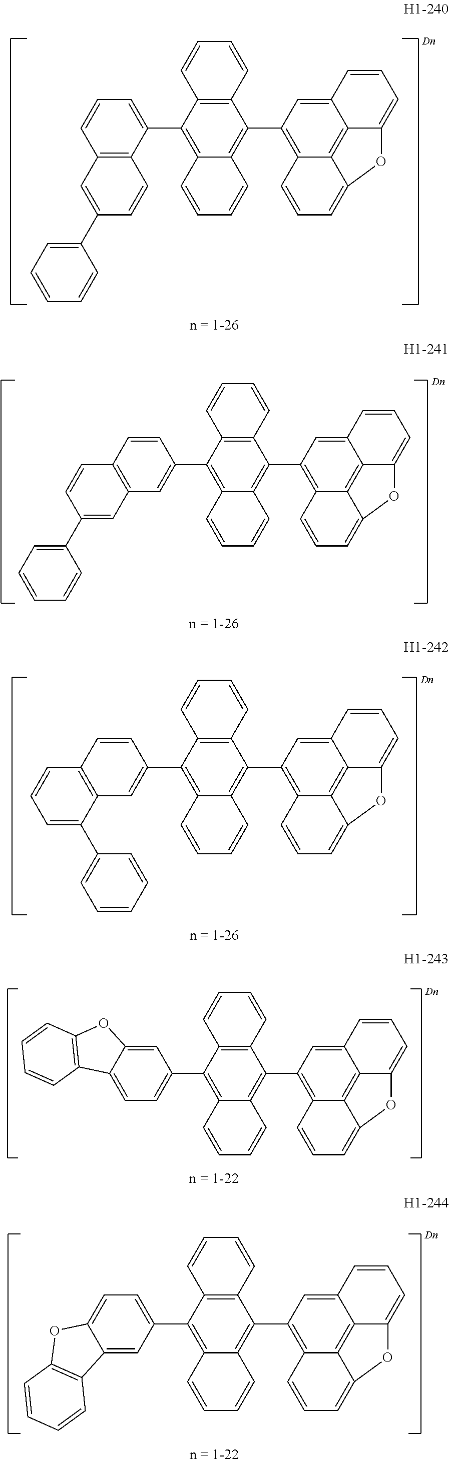

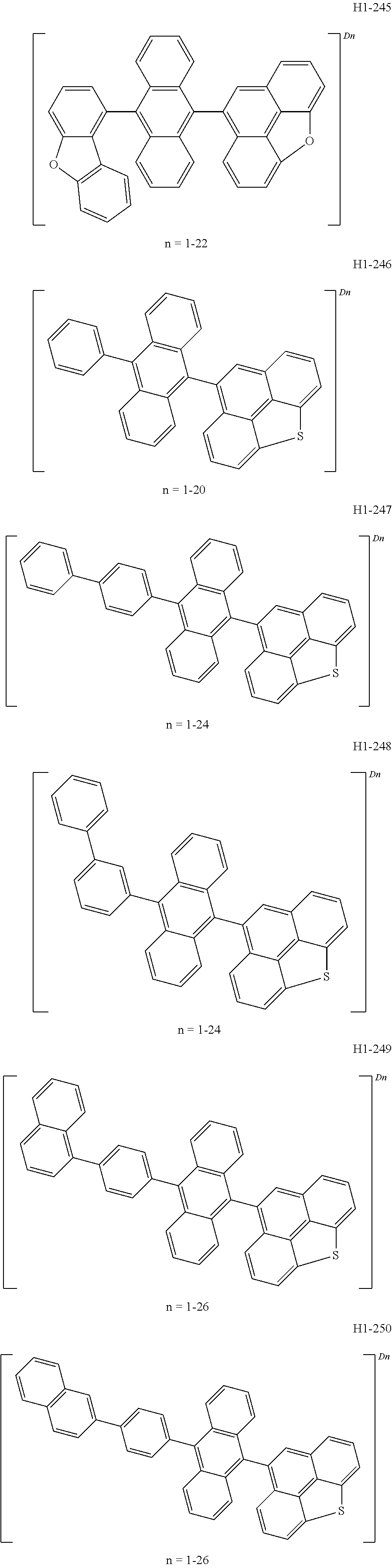

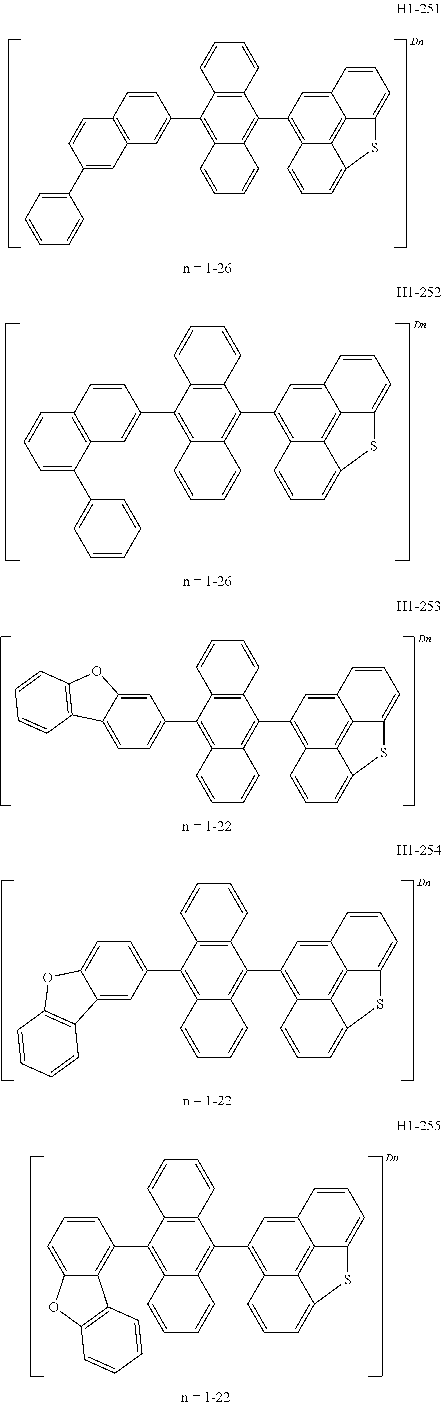

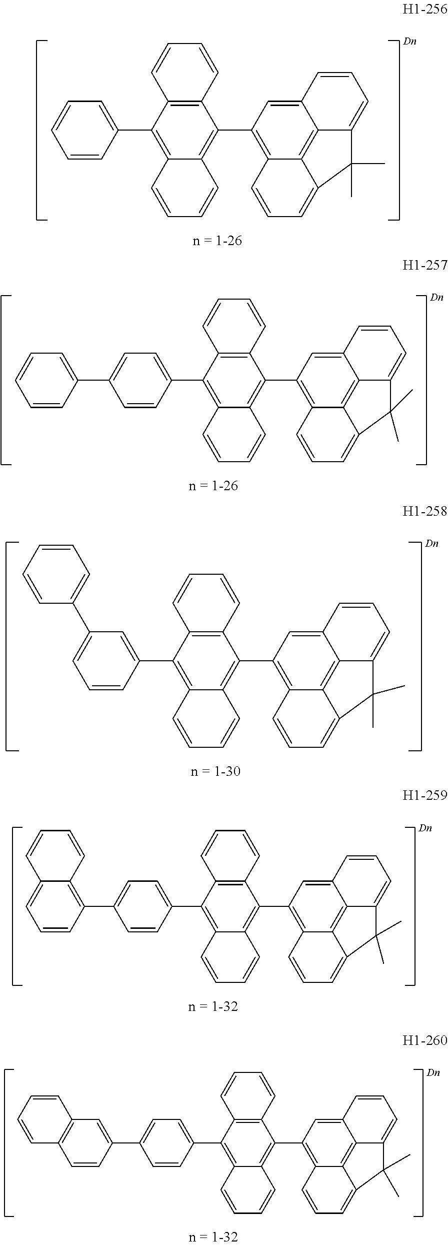

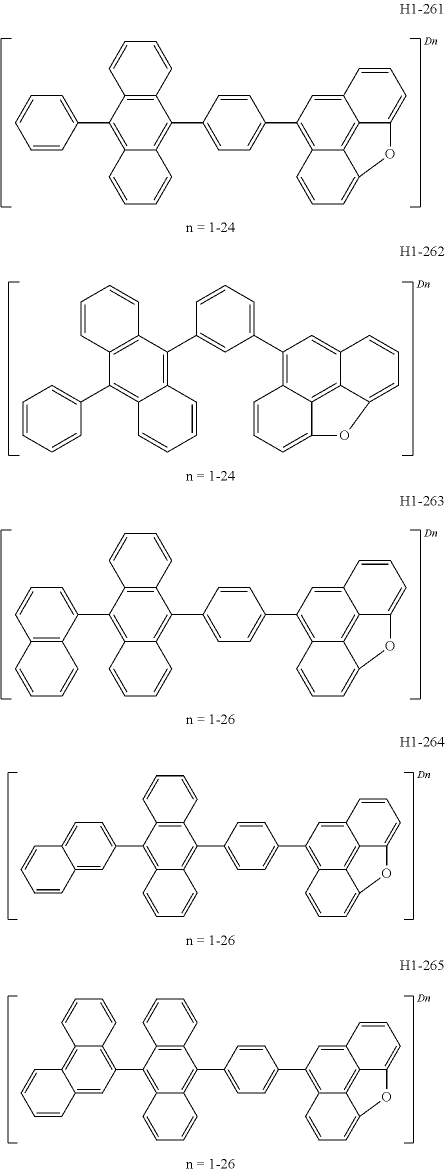

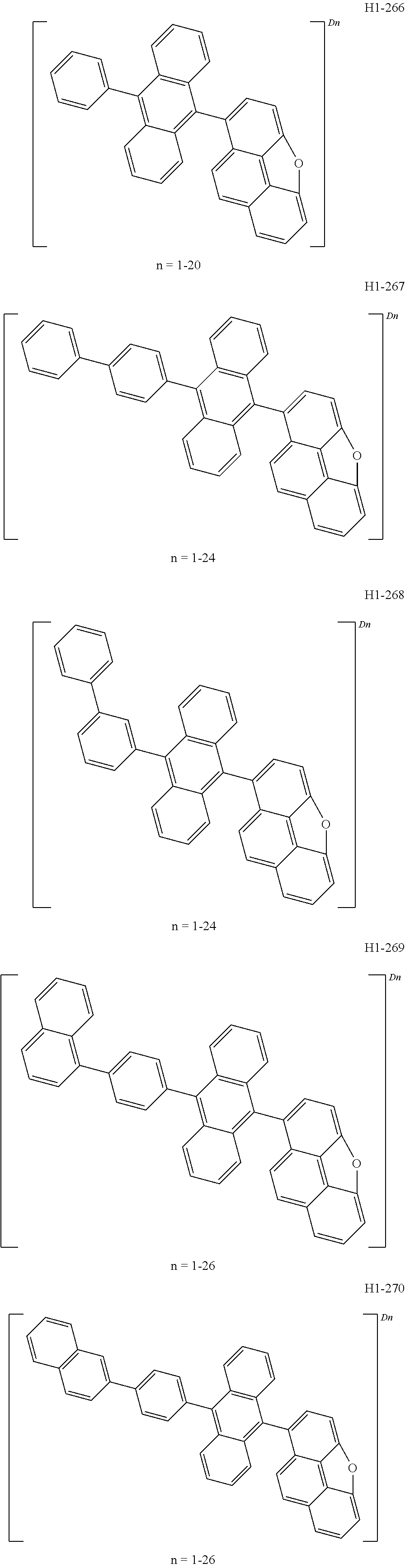

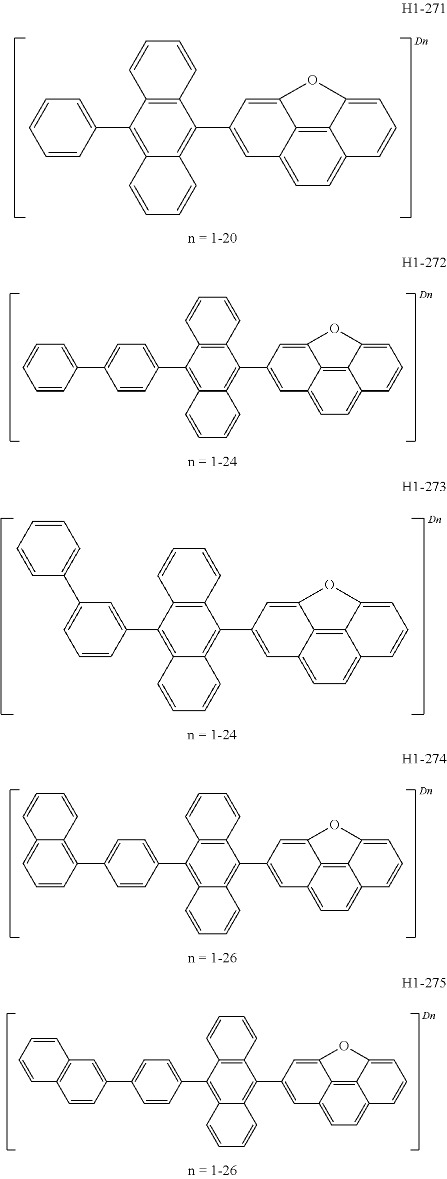

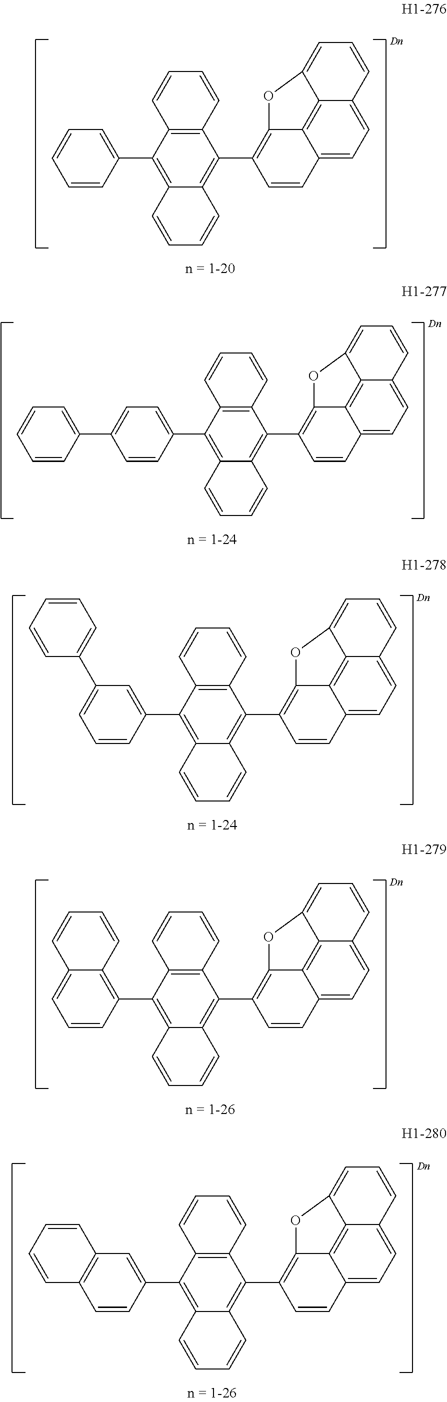

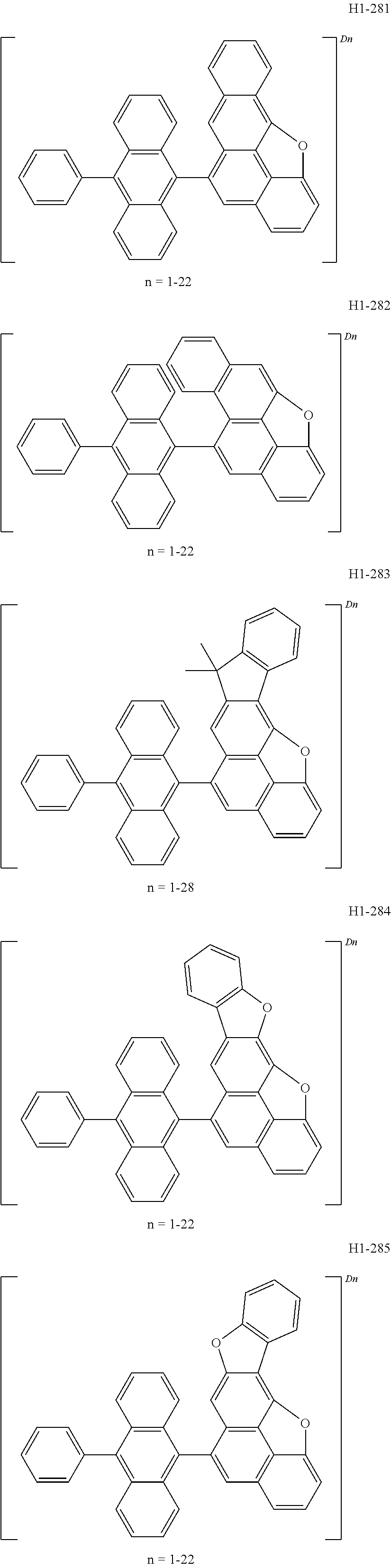

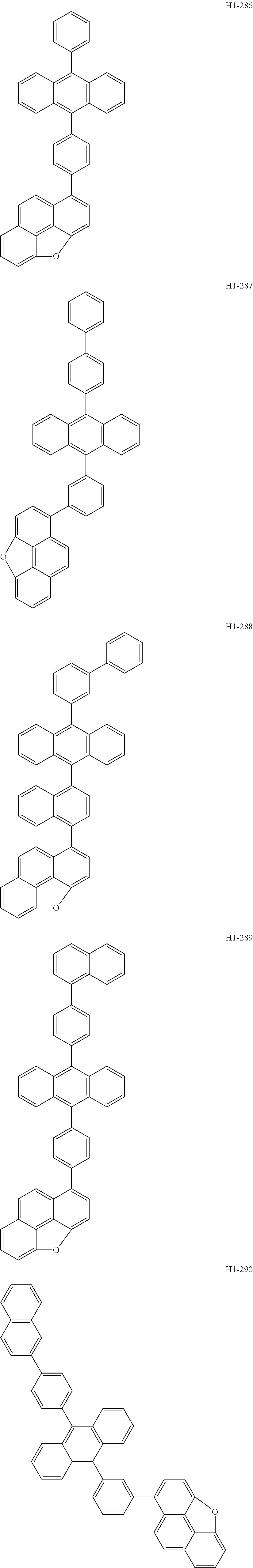

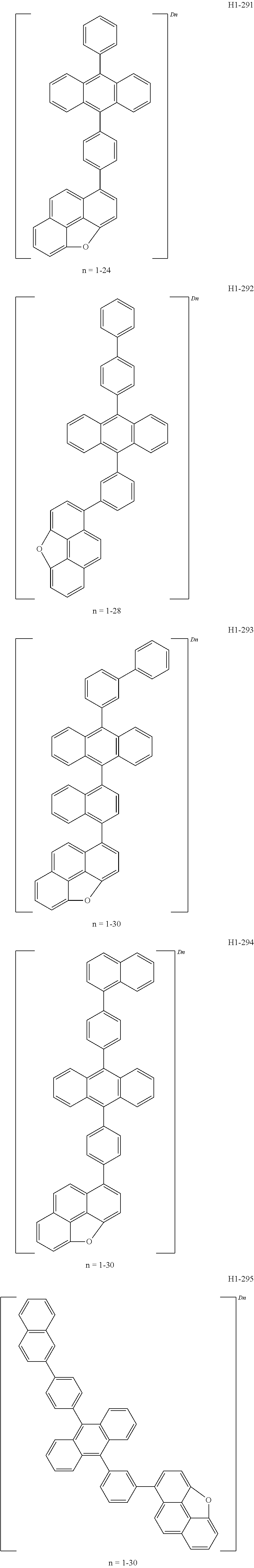

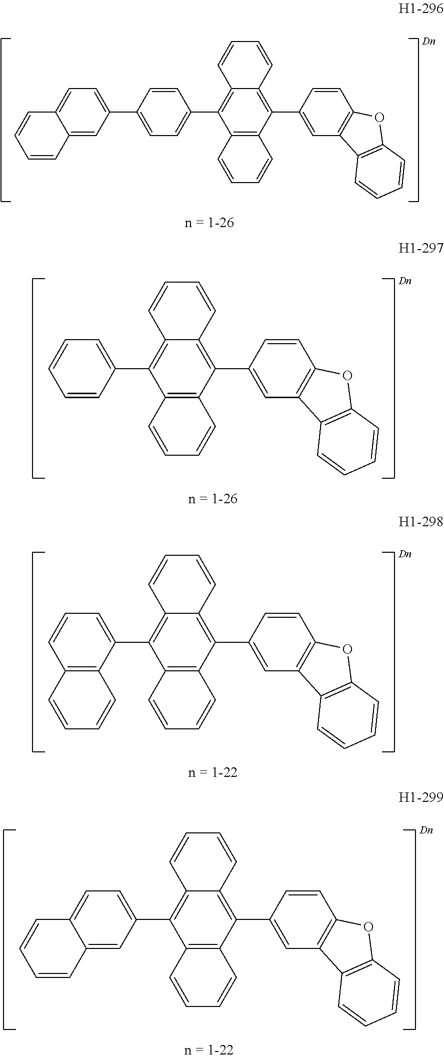

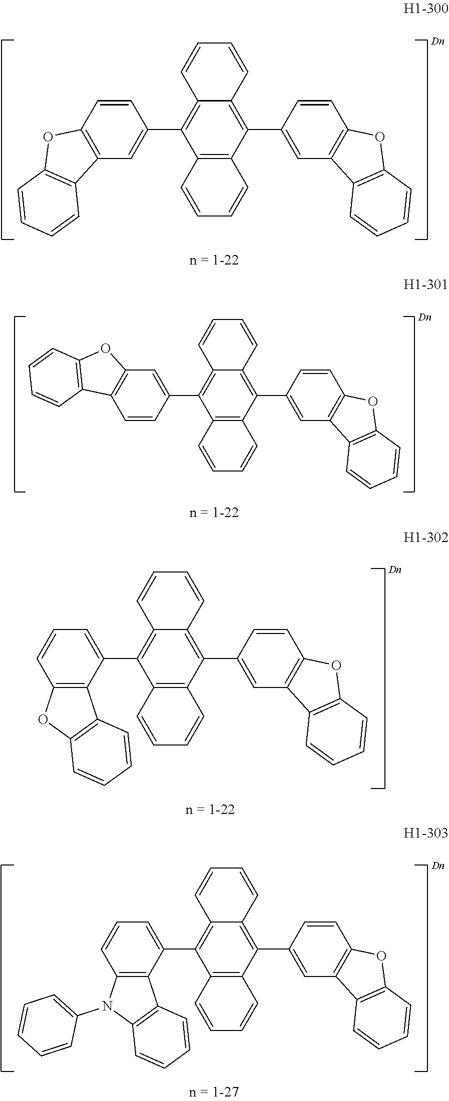

In the compounds H-56 to H-100, H1-226 to H1-285, and H1-291 to H1-303, Dn means that n number of hydrogens is replaced with deuterium, wherein n represents an integer of 1 or more, wherein the upper limit of n is determined by the number of hydrogens that can be substituted in each compound.

The light-emitting layer according to another embodiment includes at least two compounds selected from the compounds represented by the following Formulas 1-2 to 1-4.

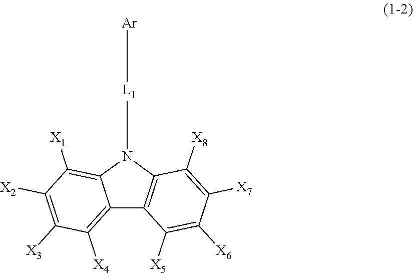

In Formula 1-2,
Ar represents a substituted or unsubstituted (C6-C30)aryl or a substituted or unsubstituted (3- to 30-membered)heteroaryl;
L₁ represents a single bond, a substituted or unsubstituted (C6-C30)arylene, or a substituted or unsubstituted (3- to 30-membered)heteroarylene;
X₁ to X₅ each independently represent, hydrogen, deuterium, halogen, cyano, carboxyl, nitro, hydroxy, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, a substituted or unsubstituted (3- to 30-membered)heteroaryl, —NX₁₁X₁₂ or —SiX₁₃X₁₄X₁₅; or the adjacent at least two of X₁ to X₈ may be linked to each other to form a ring(s); and
X₁₁ to X₁₅ each independently represent, hydrogen, deuterium, halogen, cyano, carboxyl, nitro, hydroxy, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, or a substituted or unsubstituted (3- to 30-membered)heteroaryl; or may be linked to the adjacent substituents to form a ring(s);

HAr-(L₂-Ar₂)_{d} (1-3)

In Formula 1-3.
HAr represents a substituted or unsubstituted, nitrogen-containing (3- to 20-membered)heteroaryl;
L₂ represents a single bond or a substituted or unsubstituted (C6-C30)arylene;
Ar₂ represents a substituted or unsubstituted (C6-C30)aryl or a substituted or unsubstituted (C3-C30)heteroaryl; and
d is an integer of 1 to 3, when d is 2 or more, each of (L₂-Ar₂) may be the same or different;

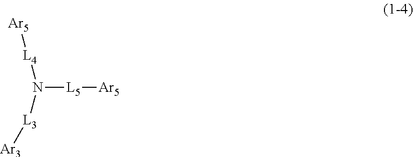

In Formula 1-4,
Ar₃ to Ar₈ each independently represent, hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or —N(Ar₁₁)(Ar₁₂);
Ar₁₁ and Ar₁₂ each independently represent, a substituted or unsubstituted (C1-C30)alkyl, a substituted or unsubstituted (C2-C30)alkenyl, a substituted or unsubstituted (C6-C30)aryl, or a substituted or unsubstituted (3- to 30-membered)heteroaryl; and
L₃ to L₅ each independently represent, a single bond, a substituted or unsubstituted (C1-C30)alkylene, a substituted or unsubstituted (C6-C30)arylene, a substituted or unsubstituted (3- to 30-membered)heteroarylene, or a substituted or unsubstituted (C3-C30)cycloalkylene;
provided that the cases where all of L₃ to L₅ are a single bond, while all of Ar₃ to Ar₅ are hydrogen are excluded.

The compound represented by Formula 1-2 according to one embodiment may be represented by the following Formula 1-2-1 or 1-2-2.

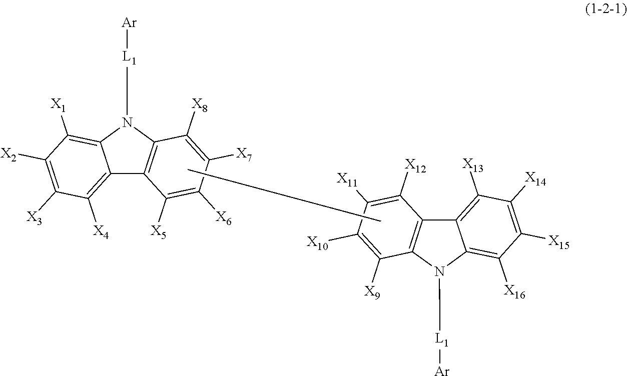

In Formula 1-2-1,
X₁ to X₅ and L₁ are as defined in Formula 1-2;
Ar and Ar′ each independently represent, a substituted or unsubstituted (C6-C30)aryl, a substituted or unsubstituted dibenzofuranyl, a substituted or unsubstituted dibenzothiophenyl, or a substituted or unsubstituted carbazolyl;
L₁′ is as defined in L₁ of Formula 1-2;
X₉ to X₁₈ are as defined in X₁ to X₈ of Formula 1-2; and
one of X₁ to X₈, and one of X₉ to X₁₆ are linked to each other to form a single bond;

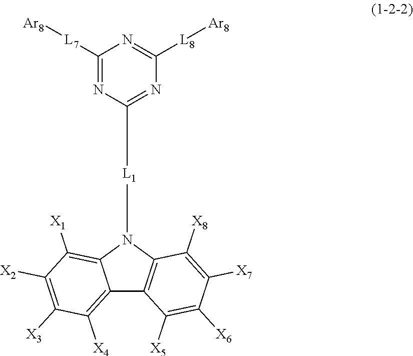

In Formula 1-2-2,
X₁ to X₅ and L₁ are as defined in Formula 1-2;
L₇ and L₈ are as defined in L₁ of Formula 1-2;
Ar₇ and Ar₈ each independently represent, a substituted or unsubstituted (C1-C30)alkyl, a substituted or unsubstituted (C6-C30)aryl, a substituted or unsubstituted (C3-C30)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino.

In Formula 1-2-1, X₁ to X₁₆ each independently may be, hydrogen, deuterium, a substituted or unsubstituted (C6-C30)aryl, or a substituted or unsubstituted (5- to 30-membered)heteroaryl; or the adjacent at least two of X₁ to X₅ may be linked to each other to form a substituted or unsubstituted polycylic aromatic ring(s). For example, X₁ to X₁₆ each independently may be hydrogen, a substituted or unsubstituted phenyl, a substituted or unsubstituted dibenzofuranyl, or a substituted or unsubstituted dibenzothiophenyl; or may be linked to the adjacent substituents to form a substituted or unsubstituted polycylic aromatic ring(s) fused with carbazole.

In Formula 1-2-1, L₁ and L₁′ each independently may be, a single bond, a substituted or unsubstituted (C6-C30)arylene, or a substituted or unsubstituted (5- to 30-membered)heteroarylene, for example, a single bond, phenylene unsubstituted or substituted with phenyl, a substituted or unsubstituted m-biphenylene, a substituted or unsubstituted naphthylene, a substituted or unsubstituted pyrimidylene, a substituted or unsubstituted carbazolylene, or a substituted or unsubstituted dibenzofuranylene.

In Formula 1-2-1, Ar and Ar′ each independently may be a substituted or unsubstituted (C6-C30)aryl or a substituted or unsubstituted (5- to 30-membered)heteroaryl, for example, phenyl unsubstituted or substituted with methyl, triphenylsilyl, dibenzofuranyl, or dibenzothiophenyl, a substituted or unsubstituted p-biphenyl, a substituted or unsubstituted m-biphenyl, a substituted or unsubstituted o-biphenyl, naphthyl unsubstituted or substituted with phenyl, a substituted or unsubstituted dimethylfluorenyl, a substituted or unsubstituted diphenylfluorenyl, a substituted or unsubstituted dimethylbenzofluorenyl, pyridyl unsubstituted or substituted with phenyl, a substituted or unsubstituted o-terphenyl, a substituted or unsubstituted p-terphenyl, a substituted or unsubstituted m-terphenyl, a substituted or unsubstituted triphenylenyl, dibenzofuranyl unsubstituted or substituted with phenyl, dibenzothiophenyl unsubstituted or substituted with phenyl, or carbazolyl unsubstituted or substituted with phenyl, naphthyl, or m-biphenyl.

In Formula 1-2-2, L₇ and L₈ each independently may be, a single bond or a substituted or unsubstituted (C6-C30)arylene, for example, a single bond or a substituted or unsubstituted phenylene.

In Formula 1-2-2, Ar₇ and Ar₈ each independently may be, a substituted or unsubstituted (C6-C30)aryl or a substituted or unsubstituted tri(C6-C30)arylsilyl, for example, a substituted or unsubstituted phenyl, a substituted or unsubstituted biphenyl, or a substituted or unsubstituted triphenylsilyl.

In Formula 1-3, HAr may be a substituted or unsubstituted nitrogen-containing (5- to 20-membered)heteroaryl, for example, triazinyl.

In Formula 1-3, L₂ may be a single bond, or a substituted or unsubstituted (C6-C30)arylene, for example, a single bond, a substituted or unsubstituted phenylene, a substituted or unsubstituted biphenylene, or a substituted or unsubstituted naphthylene.

In Formula 1-3, Ar₂ may be a substituted or unsubstituted (C6-C30)aryl or a substituted or unsubstituted (C5-C30)heteroaryl, for example, phenyl unsubstituted or substituted with naphthyl, naphthyl unsubstituted or substituted with phenyl or biphenyl, a substituted or unsubstituted m-biphenyl, a substituted or unsubstituted p-biphenyl, a substituted or unsubstituted p-terphenyl, a substituted or unsubstituted phenanthrenyl, a substituted or unsubstituted chrysenyl, a substituted or unsubstituted benzophenanthrenyl, or dibenzofuranyl unsubstituted or substituted with at least one of phenyl, naphthyl, phenylnaphthyl, naphthylphenyl, and dibenzofuranyl. Wherein, the substituted group may be further substituted with at least one deuterium.

In Formula 1-4, L₃ to L₅ each independently may be, a single bond, a substituted or unsubstituted (C6-C30)arylene, or a substituted or unsubstituted (5- to 30-membered)heteroarylene. For example, L₃ to L₅ each independently may be, a single bon, phenylene unsubstituted or substituted with phenyl or pyridyl, a substituted or unsubstituted naphthylene, a substituted or unsubstituted phenanthrenylene, a substituted or unsubstituted o-biphenylene, a substituted or unsubstituted m-biphenylene, a substituted or unsubstituted p-biphenylene, a substituted or unsubstituted pyridylene, or a substituted or unsubstituted carbazolylene.

In Formula 1-4, Ar₃ to Ar₅ each independently may be, a substituted or unsubstituted (C1-C30)alkyl, a substituted or unsubstituted (C6-C30)aryl, a substituted or unsubstituted (5- to 30-membered)heteroaryl, a substituted or unsubstituted tri(C6-C30)arylsilyl, or —N(Ar₁₁)(Ar₁₂). Wherein, Ar₁₁ and Ar₁₂ each independently may be, a substituted or unsubstituted (C6-C30)aryl or a substituted or unsubstituted (5- to 30-membered)heteroaryl.

For example, Ar₃ to Ar₅ each independently may be, phenyl unsubstituted or substituted with deuterium or cyano, isopropyl unsubstituted or substituted with phenyl, a substituted or unsubstituted naphthyl, a substituted or unsubstituted m-biphenyl, a substituted or unsubstituted p-biphenyl, a substituted or unsubstituted o-terphenyl, a substituted or unsubstituted m-terphenyl, a substituted or unsubstituted p-terphenyl, a substituted or unsubstituted o-quarterphenyl, a substituted or unsubstituted phenanthrenyl, a substituted or unsubstituted chrysenyl, fluorenyl unsubstituted or substituted with at least one methyl, fluorenyl unsubstituted or substituted with at least one phenyl, a substituted or unsubstituted 9,9,10,10-teteramethylphenanthrenyl, a substituted or unsubstituted triphenylsilyl, a substituted or unsubstituted pyridyl, a substituted or unsubstituted carbazolyl, dibenzofuranyl unsubstituted or substituted with deuterium, a substituted or unsubstituted dibenzothiophenyl, a substituted or unsubstituted phenanthrolinyl, a substituted or unsubstituted dibenzoselenophenyl, or a substituted or unsubstituted naphthobenzoselenophenyl. Wherein, the substituted groups may be further substituted with at least one of deuterium, methyl, and phenyl.

For example, Ar₁ and Ar₁₂ each independently may be, phenyl unsubstituted or substituted with diphenylamino, a substituted or unsubstituted naphthyl, a substituted or unsubstituted phenanthrenyl, a substituted or unsubstituted o-biphenyl, a substituted or unsubstituted m-biphenyl, a substituted or unsubstituted p-biphenyl, a substituted or unsubstituted o-terphenyl, a substituted or unsubstituted m-terphenyl, a substituted or unsubstituted p-terphenyl, a substituted or unsubstituted pyridyl, a substituted or unsubstituted dibenzothiophenyl, or a substituted or unsubstituted dibenzofuranyl.

According to one embodiment. Ar₂ of Formula 1-3, and at least one of Ar₃ to Ar₅ of Formula 1-4 may be represented by any one of the following Formulas 1-3-1 to 1-3-3.

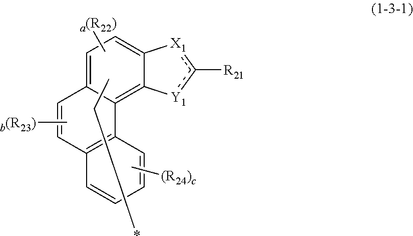

In Formula 1-3-1,
X₁ and Y₁ each independently represent, —N═, —NR₂₅—, —O— or —S—; provided that any one of X₁ and Y₁ is —N═, and other of X₁ and Y₁ is —NR₂₅—, —O— or —S—;
R₂₁ represents a substituted or unsubstituted (C6-C30)aryl or a substituted or unsubstituted (3- to 30-membered)heteroaryl;
R₂₂ to R₂₅ each independently represent, hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or may be linked to the adjacent substituents to form a ring(s);
a and b each independently represent, an integer of 1 or 2, and c is an integer of 1 to 4, when a to c are 2 or more, each of R₂₂ to each of R₂₄ may be the same or different; and
* represents a linking site with L₂ of Formula 1-3, or a linking site with L₃ to L₅ of Formula 1-4;

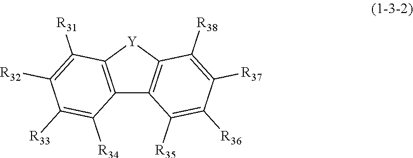

In Formula 1-3-2,
Y represents —O—, —S, or —NR₃₉;
R₃₉ represents a substituted or unsubstituted (C6-C30)aryl; and
R₃₁ to Ram each independently represent, a linking site with L₂ of Formula 1-3, or a linking site with L₈ to L₅ of Formula 1-4; or represent, hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or may be linked to the adjacent substituents to form a ring(s);

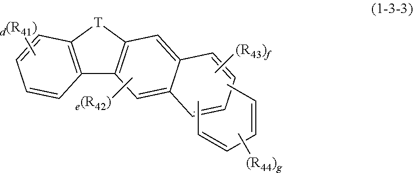

In Formula 1-3-3,
T represents —O—, —S—, —CR₄₅R₄₆, —NR₄₇, or —Se—;
R₄₅ to R₄₇ each independently represent, hydrogen, deuterium, halogen, a substituted or unsubstituted (C1-C30)alkyl, or a substituted or unsubstituted (C6-C30)aryl;
R₄₁ to R₄₄ each independently represent hydrogen, deuterium, halogen, a substituted or unsubstituted (C1-C30)alkyl, a substituted or unsubstituted (C6-C30)aryl, a substituted or unsubstituted (3- to 30-membered)heteroaryl, or —N(Ar₂₁)(Ar₂₂); or may be linked to the adjacent substituents to form a ring(s);
Ar₂₁ and Ar₂₂ each independently represent, a substituted or unsubstituted (C1-C30)alkyl, a substituted or unsubstituted (C2-C30)alkenyl, a substituted or unsubstituted (C6-C30)aryl, or a substituted or unsubstituted (3- to 30-membered)heteroaryl;
d and g each independently represent, an integer of 1 to 4, and e and f each independently represent, an integer of 1 or 2;
when d to g are 2 or more, each of R₄₁ to R₄₄ may be the same or different; and
* represent a linking site with L₂ of Formula 1-3, or a linking site with L₃ to L₅ of Formula 1-4.

In Formula 1-3-1, R₂₁ may be a substituted or unsubstituted (C6-C30)aryl or a substituted or unsubstituted (5- to 30-membered)heteroaryl, for example, phenyl unsubstituted or substituted with deuterium, a substituted or unsubstituted naphthyl, a substituted or unsubstituted o-biphenyl, a substituted or unsubstituted m-biphenyl, a substituted or unsubstituted p-biphenyl, or a substituted or unsubstituted pyridyl.

In Formula 1-3-1, R₂₂ to R₂₅ each independently may be hydrogen, deuterium, halogen, cyano, a substituted or unsubstituted (C6-C30)aryl or a substituted or unsubstituted (5- to 30-membered)heteroaryl, for example, hydrogen, deuterium, phenyl unsubstituted or substituted with naphthyl or triphenylsilyl, a substituted or unsubstituted naphthyl, a substituted or unsubstituted o-biphenyl, a substituted or unsubstituted m-biphenyl, a substituted or unsubstituted p-biphenyl, a substituted or unsubstituted p-terphenyl, fluorenyl unsubstituted or substituted with at least one methyl, a substituted or unsubstituted dibenzofuranyl, a substituted or unsubstituted dibenzothiophenyl, a substituted or unsubstituted benzonaphthofuranyl, or a substituted or unsubstituted benzonaphthothiophenyl.

In Formula 1-3-2, one of R₃₁ to R₃₆ may be a linking site with L₂ of Formula 1-3 or a linking site with L to L₅ of Formula 1-4.

In Formula 1-3-3, T may be —O— or —S—.

In Formula 1-3-3, R₄₁ to R₄₄ each independently may be, hydrogen, deuterium, a substituted or unsubstituted (C6-C30)aryl, or —N(Ar₂₁)(Ar₂₂), for example, hydrogen, deuterium, a substituted or unsubstituted phenyl, a substituted or unsubstituted p-biphenyl, a substituted or unsubstituted m-biphenyl, a substituted or unsubstituted naphthyl, or a substituted or unsubstituted diphenylamine. For example, Ar₂₁ and Ar₂ each independently may be, a substituted or unsubstituted phenyl.

Formula 1-3-3 according to one embodiment may be represented by any one of the following Formulas 1-3-4 to 1-3-7.

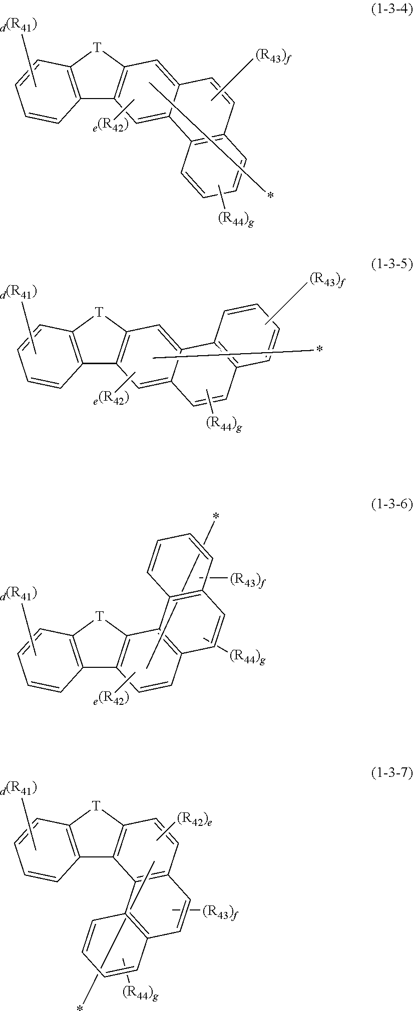

In Formulas 1-3-4 to 1-3-7.

T, R₄₁ to R₄₄, and d to g are as defined in Formula 1-3-3.

According to one embodiment, the compound represented by Formulas 1-2 to 1-4 may comprise at least one deuterium.

According to one embodiment, the compound represented by Formulas 1-2 to 1-4 may be more specifically illustrated by the following compounds, but is not limited thereto.

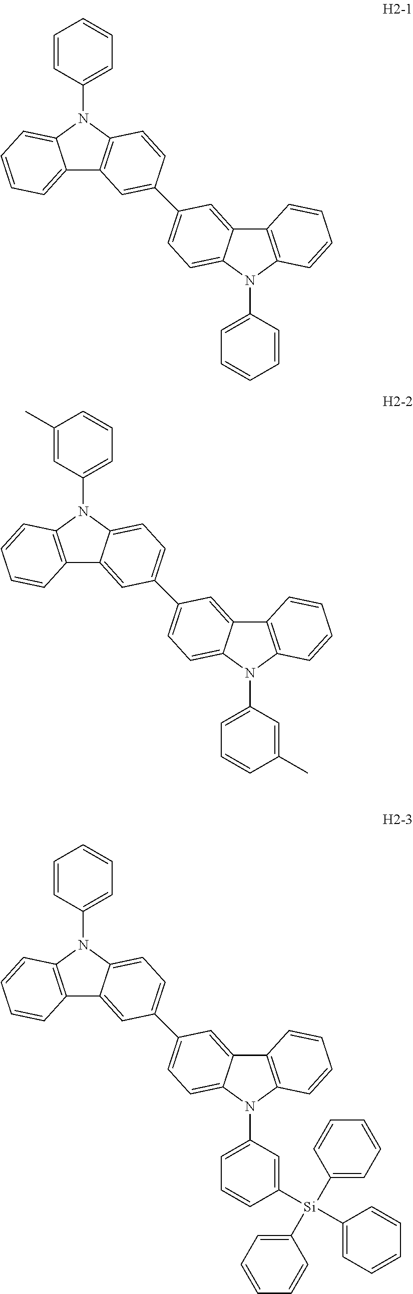 [SMILES: c1ccc(-n2c3ccccc3c3cc(-c4ccc5c(c4)c4ccccc4n5-c4ccccc4)ccc32)cc1 ; Cc1cccc(-n2c3ccccc3c3cc(-c4ccc5c(c4)c4ccccc4n5-c4cccc(C)c4)ccc32)c1 ; c1ccc(-n2c3ccccc3c3cc(-c4ccc5c(c4)c4ccccc4n5-c4cccc([Si](c5ccccc5)(c5ccccc5)c5ccccc5)c4)ccc32)cc1]

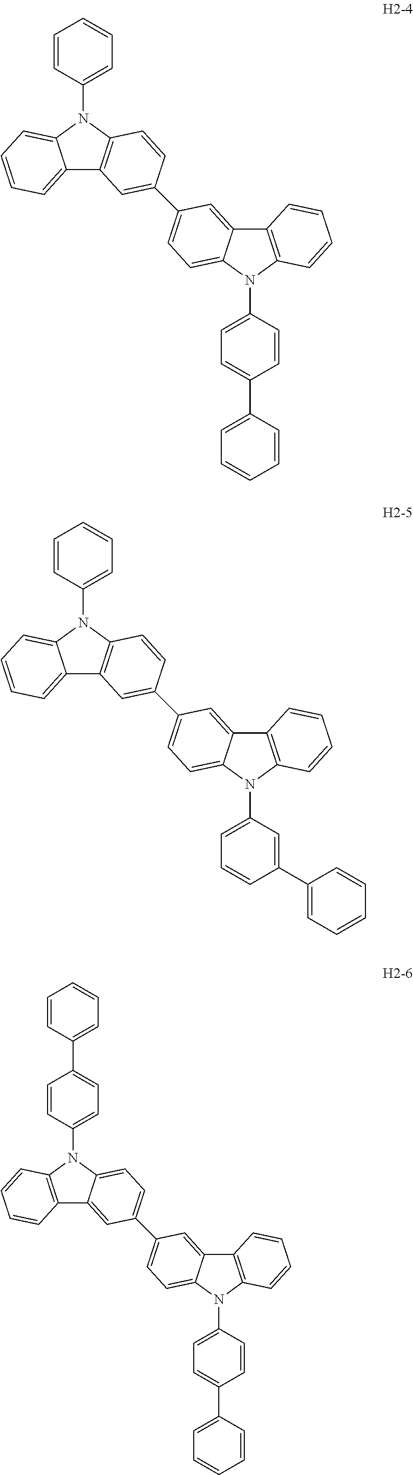 [SMILES: c1ccc(-c2ccc(-n3c4ccccc4c4cc(-c5ccc6c(c5)c5ccccc5n6-c5ccccc5)ccc43)cc2)cc1 ; c1ccc(-c2cccc(-n3c4ccccc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5ccc(-c6ccccc6)cc5)ccc43)cc2)cc1]

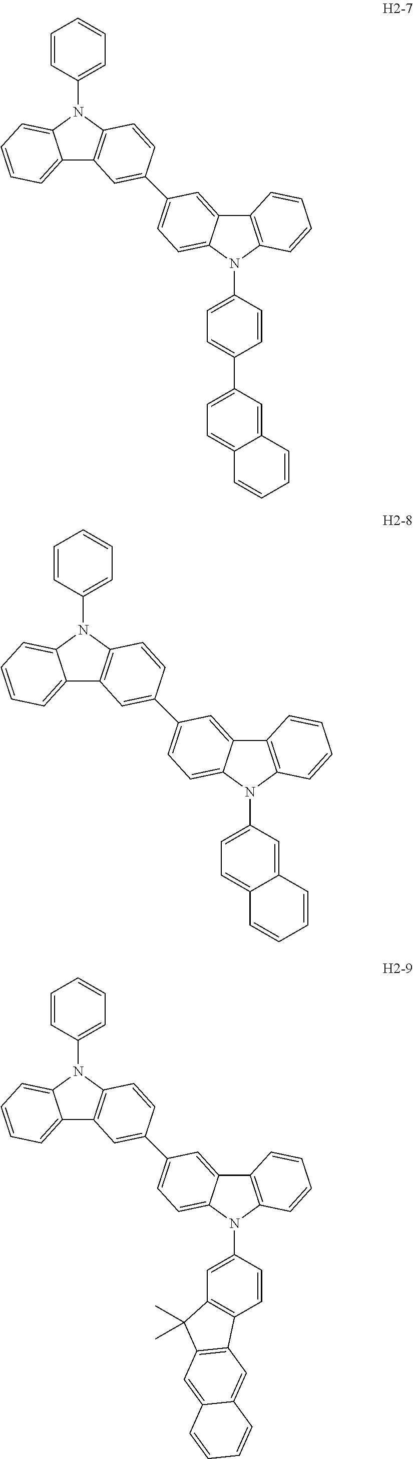 [SMILES: c1ccc(-n2c3ccccc3c3cc(-c4ccc5c(c4)c4ccccc4n5-c4ccc(-c5ccc6ccccc6c5)cc4)ccc32)cc1 ; c1ccc(-n2c3ccccc3c3cc(-c4ccc5c(c4)c4ccccc4n5-c4ccc5ccccc5c4)ccc32)cc1 ; CC1(C)c2cc(-n3c4ccccc4c4cc(-c5ccc6c(c5)c5ccccc5n6-c5ccccc5)ccc43)ccc2-c2cc3ccccc3cc21]

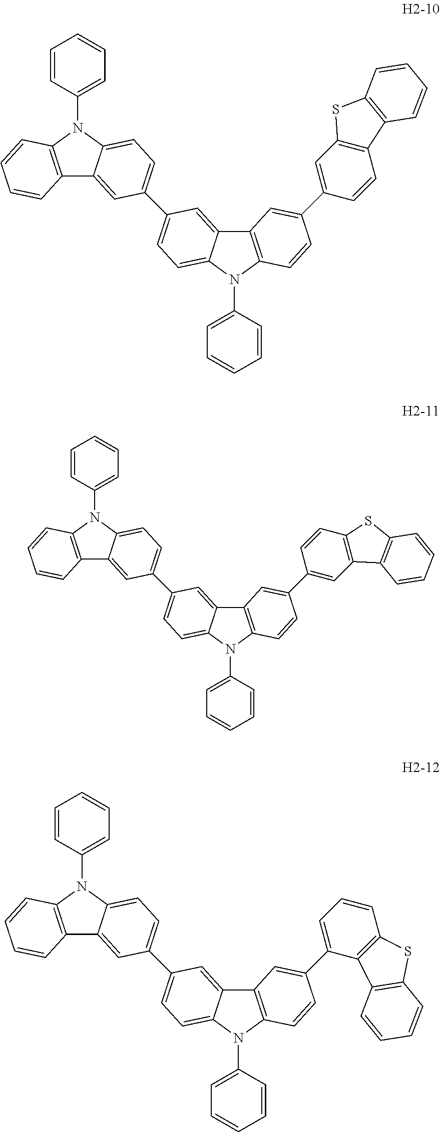 [SMILES: c1ccc(-n2c3ccccc3c3cc(-c4ccc5c(c4)c4cc(-c6ccc7c(c6)sc6ccccc67)ccc4n5-c4ccccc4)ccc32)cc1 ; c1ccc(-n2c3ccccc3c3cc(-c4ccc5c(c4)c4cc(-c6ccc7sc8ccccc8c7c6)ccc4n5-c4ccccc4)ccc32)cc1 ; c1ccc(-n2c3ccccc3c3cc(-c4ccc5c(c4)c4cc(-c6cccc7sc8ccccc8c67)ccc4n5-c4ccccc4)ccc32)cc1]

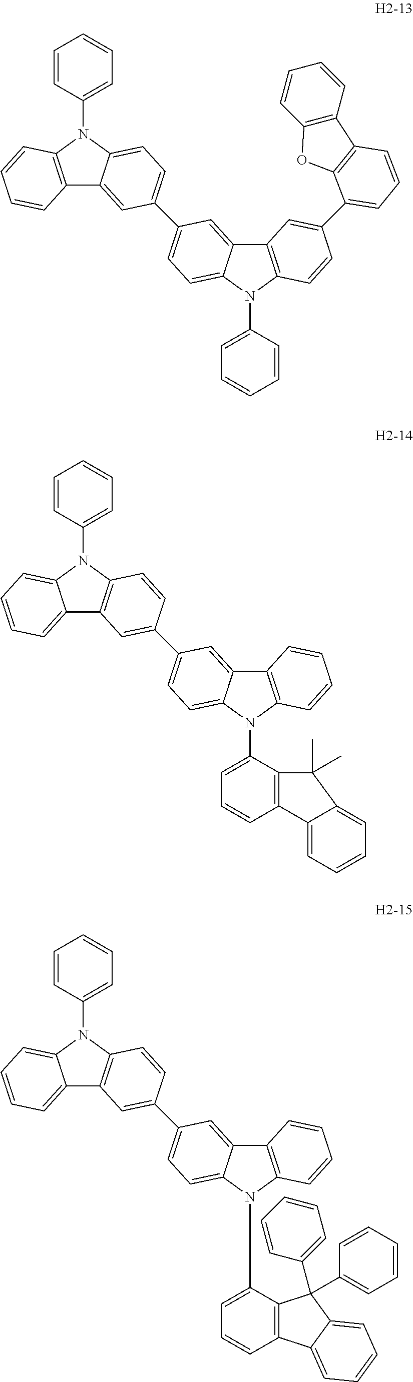 [SMILES: c1ccc(-n2c3ccccc3c3cc(-c4ccc5c(c4)c4cc(-c6cccc7c6oc6ccccc67)ccc4n5-c4ccccc4)ccc32)cc1 ; CC1(C)c2ccccc2-c2cccc(-n3c4ccccc4c4cc(-c5ccc6c(c5)c5ccccc5n6-c5ccccc5)ccc43)c21 ; c1ccc(-n2c3ccccc3c3cc(-c4ccc5c(c4)c4ccccc4n5-c4cccc5c4C(c4ccccc4)(c4ccccc4)c4ccccc4-5)ccc32)cc1]

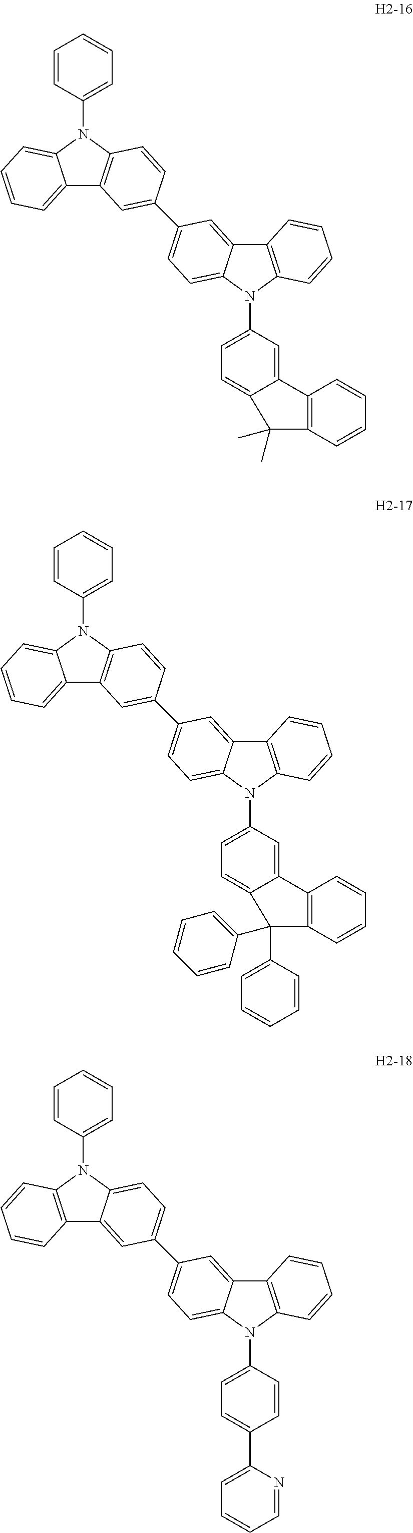 [SMILES: CC1(C)c2ccccc2-c2cc(-n3c4ccccc4c4cc(-c5ccc6c(c5)c5ccccc5n6-c5ccccc5)ccc43)ccc21 ; c1ccc(-n2c3ccccc3c3cc(-c4ccc5c(c4)c4ccccc4n5-c4ccc5c(c4)-c4ccccc4C5(c4ccccc4)c4ccccc4)ccc32)cc1 ; c1ccc(-n2c3ccccc3c3cc(-c4ccc5c(c4)c4ccccc4n5-c4ccc(-c5ccccn5)cc4)ccc32)cc1]

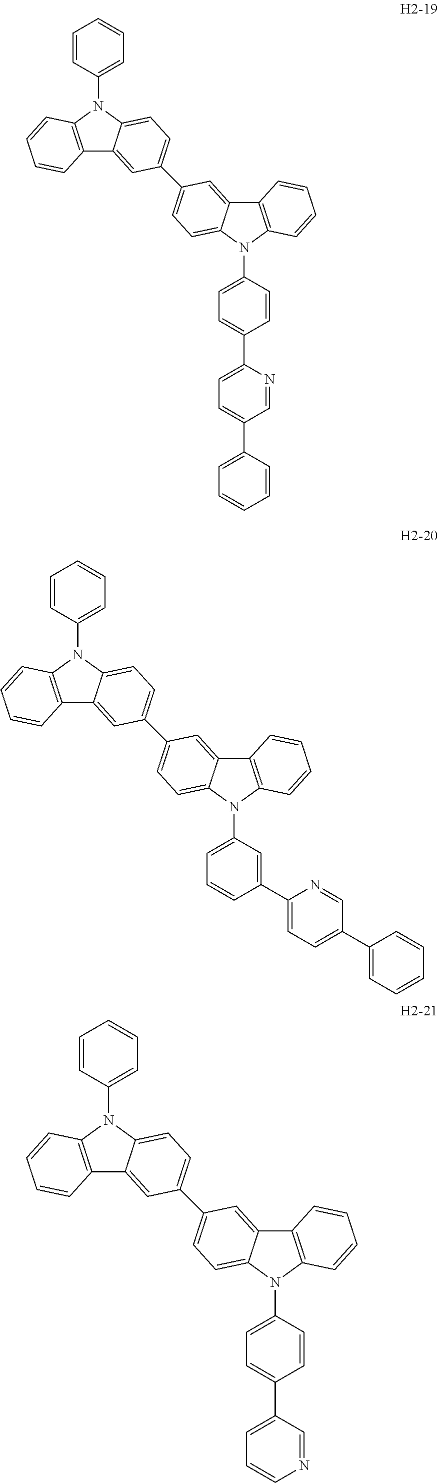 [SMILES: c1ccc(-c2ccc(-c3ccc(-n4c5ccccc5c5cc(-c6ccc7c(c6)c6ccccc6n7-c6ccccc6)ccc54)cc3)nc2)cc1 ; c1ccc(-c2ccc(-c3cccc(-n4c5ccccc5c5cc(-c6ccc7c(c6)c6ccccc6n7-c6ccccc6)ccc54)c3)nc2)cc1 ; c1ccc(-n2c3ccccc3c3cc(-c4ccc5c(c4)c4ccccc4n5-c4ccc(-c5cccnc5)cc4)ccc32)cc1]

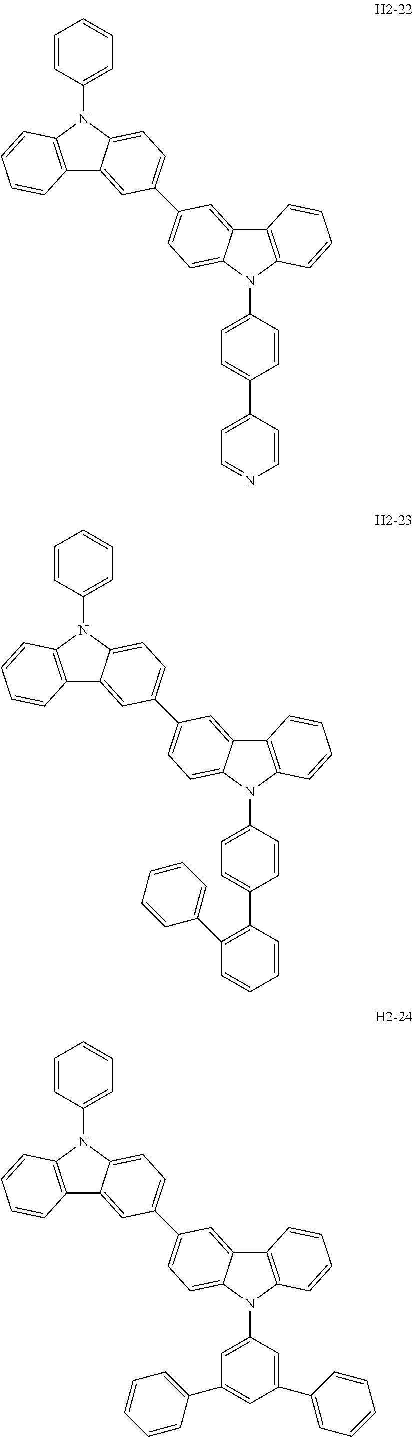 [SMILES: c1ccc(-n2c3ccccc3c3cc(-c4ccc5c(c4)c4ccccc4n5-c4ccc(-c5ccncc5)cc4)ccc32)cc1 ; c1ccc(-c2ccccc2-c2ccc(-n3c4ccccc4c4cc(-c5ccc6c(c5)c5ccccc5n6-c5ccccc5)ccc43)cc2)cc1 ; c1ccc(-c2cc(-c3ccccc3)cc(-n3c4ccccc4c4cc(-c5ccc6c(c5)c5ccccc5n6-c5ccccc5)ccc43)c2)cc1]

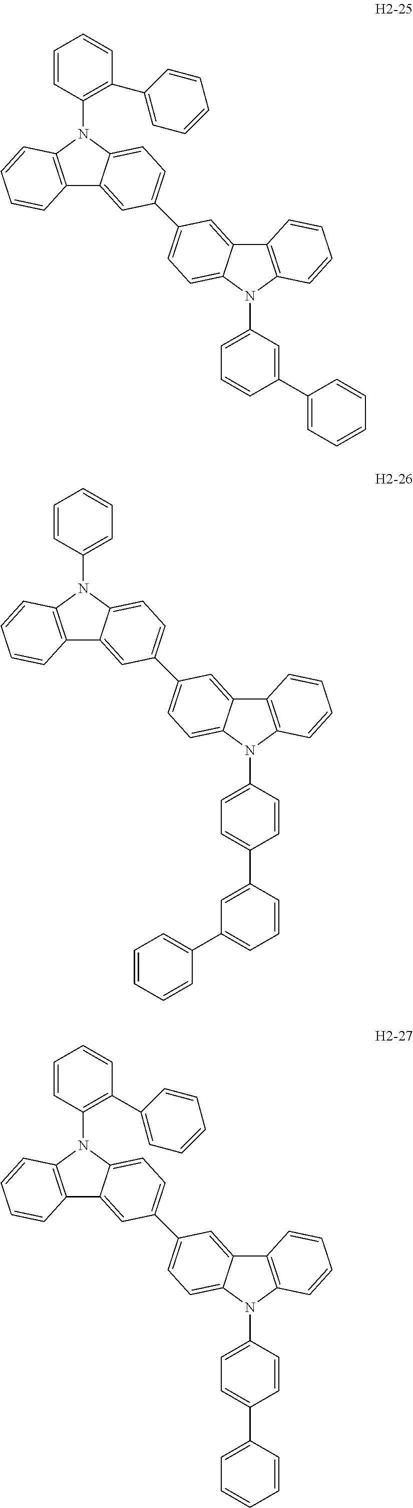 [SMILES: c1ccc(-c2cccc(-n3c4ccccc4c4cc(-c5ccc6c(c5)c5ccccc5n6-c5ccccc5-c5ccccc5)ccc43)c2)cc1 ; c1ccc(-c2cccc(-c3ccc(-n4c5ccccc5c5cc(-c6ccc7c(c6)c6ccccc6n7-c6ccccc6)ccc54)cc3)c2)cc1 ; c1ccc(-c2ccc(-n3c4ccccc4c4cc(-c5ccc6c(c5)c5ccccc5n6-c5ccccc5-c5ccccc5)ccc43)cc2)cc1]

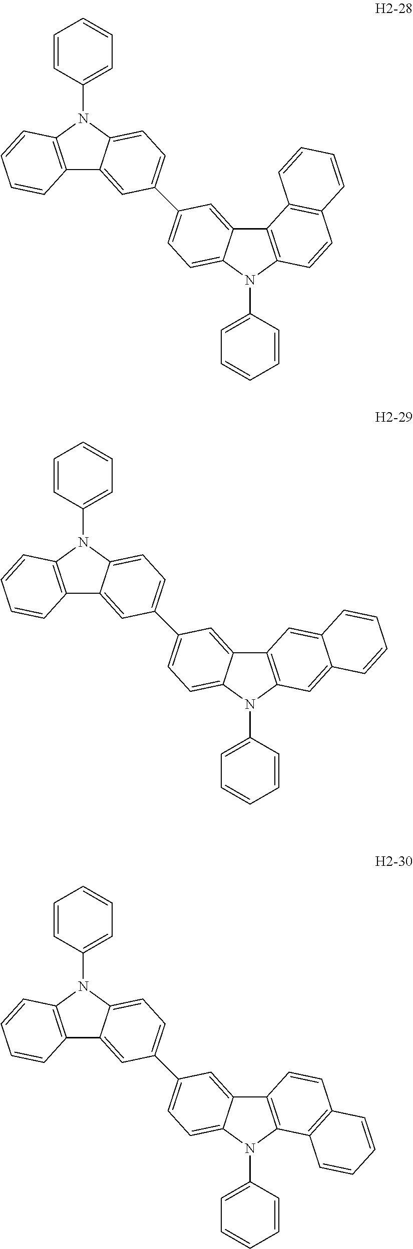 [SMILES: c1ccc(-n2c3ccccc3c3cc(-c4ccc5c(c4)c4c6ccccc6ccc4n5-c4ccccc4)ccc32)cc1 ; c1ccc(-n2c3ccccc3c3cc(-c4ccc5c(c4)c4cc6ccccc6cc4n5-c4ccccc4)ccc32)cc1 ; c1ccc(-n2c3ccccc3c3cc(-c4ccc5c(c4)c4ccc6ccccc6c4n5-c4ccccc4)ccc32)cc1]

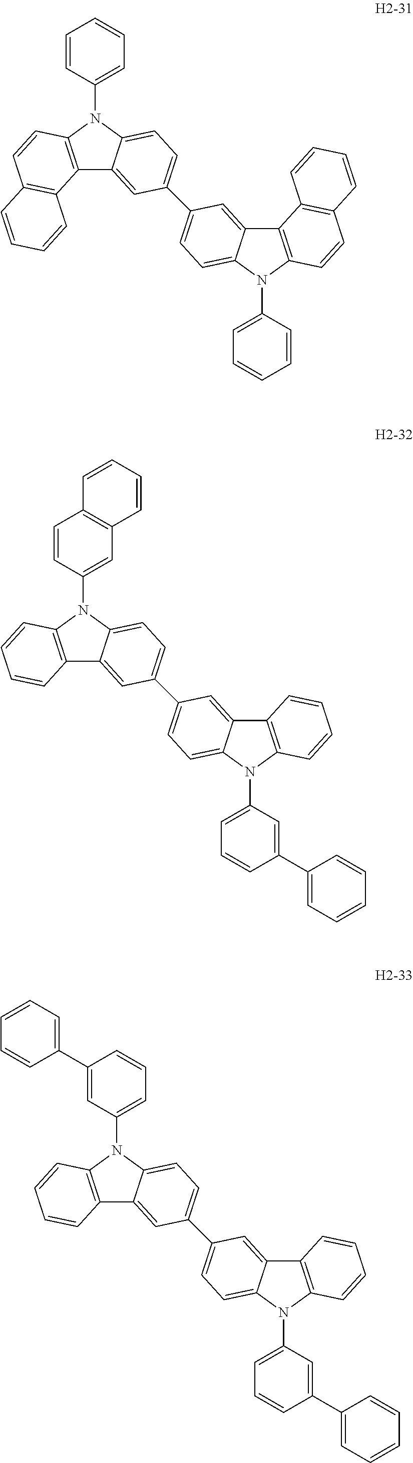 [SMILES: c1ccc(-n2c3ccc(-c4ccc5c(c4)c4c6ccccc6ccc4n5-c4ccccc4)cc3c3c4ccccc4ccc32)cc1 ; c1ccc(-c2cccc(-n3c4ccccc4c4cc(-c5ccc6c(c5)c5ccccc5n6-c5ccc6ccccc6c5)ccc43)c2)cc1 ; c1ccc(-c2cccc(-n3c4ccccc4c4cc(-c5ccc6c(c5)c5ccccc5n6-c5cccc(-c6ccccc6)c5)ccc43)c2)cc1]

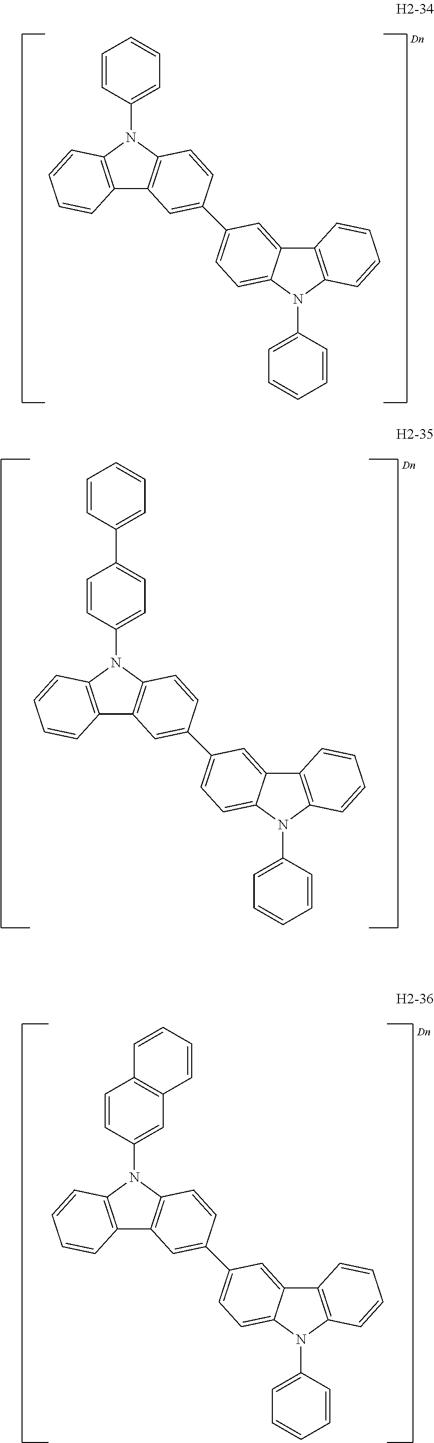

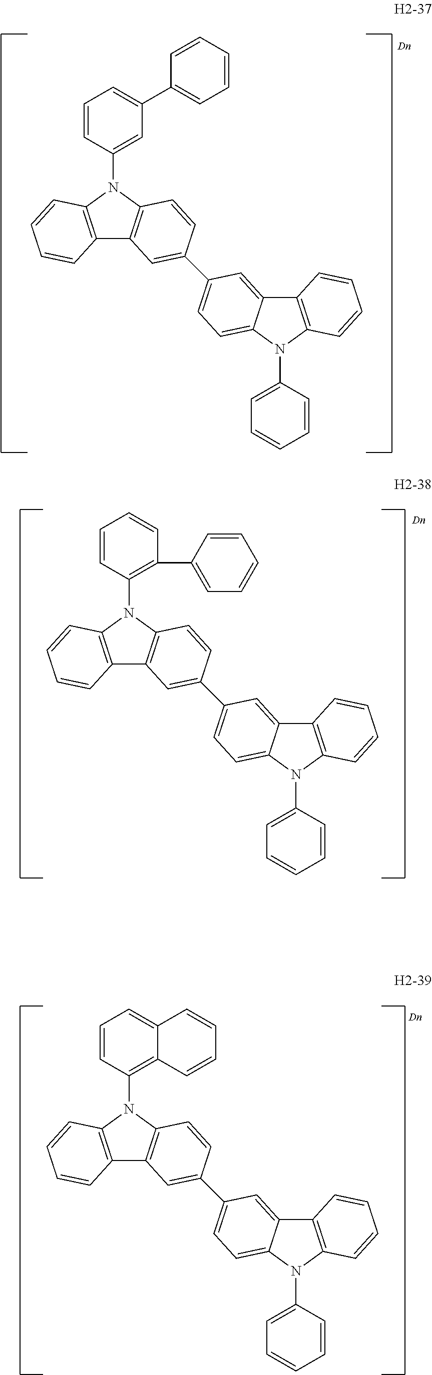

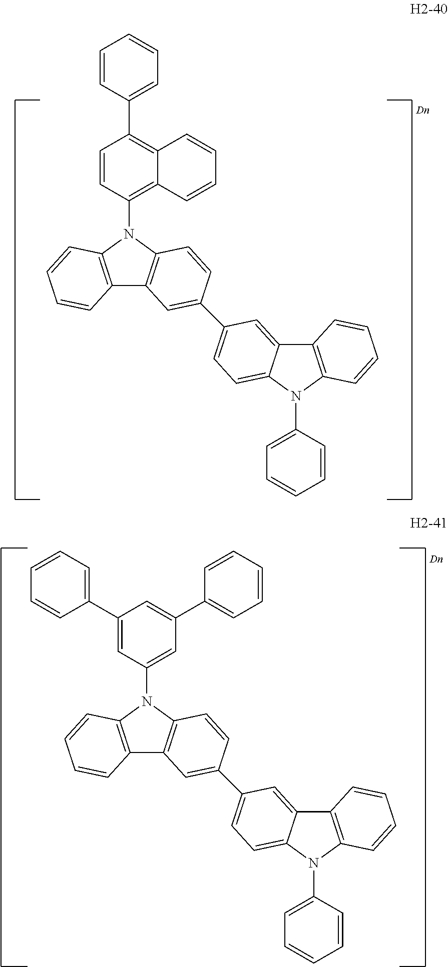

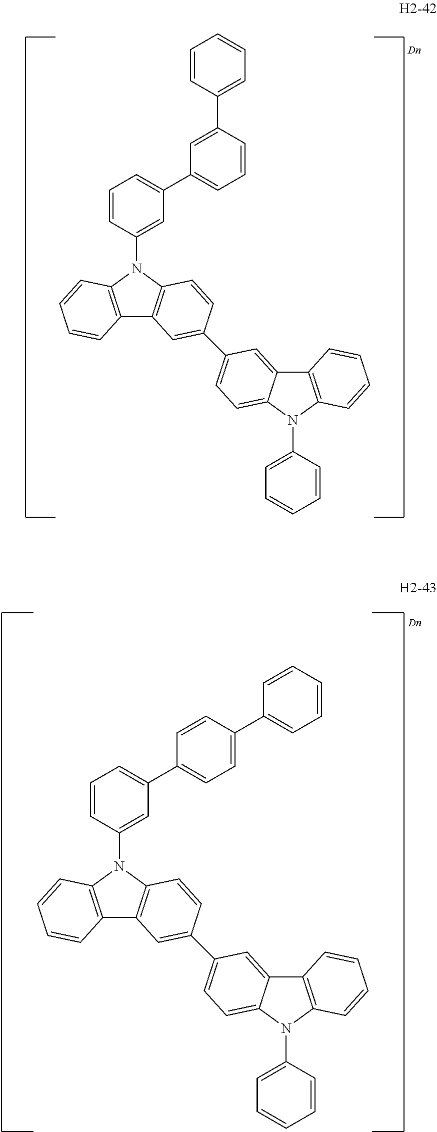

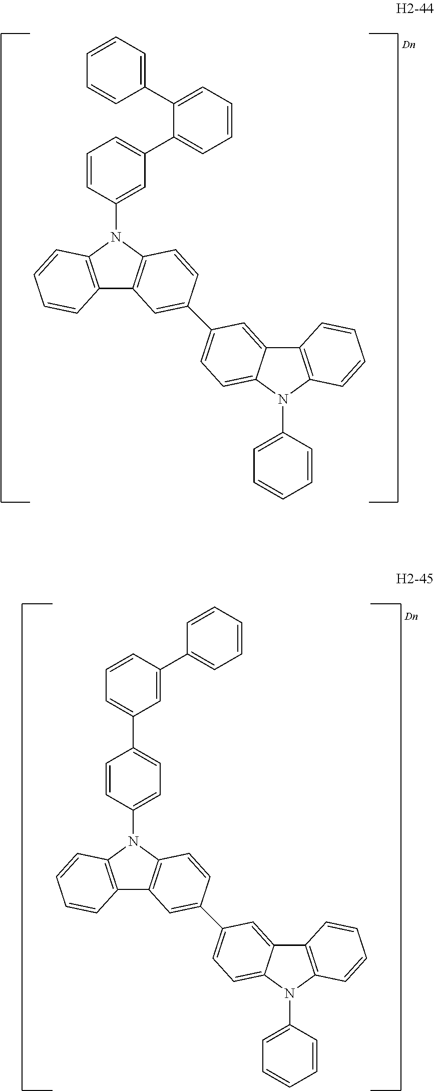

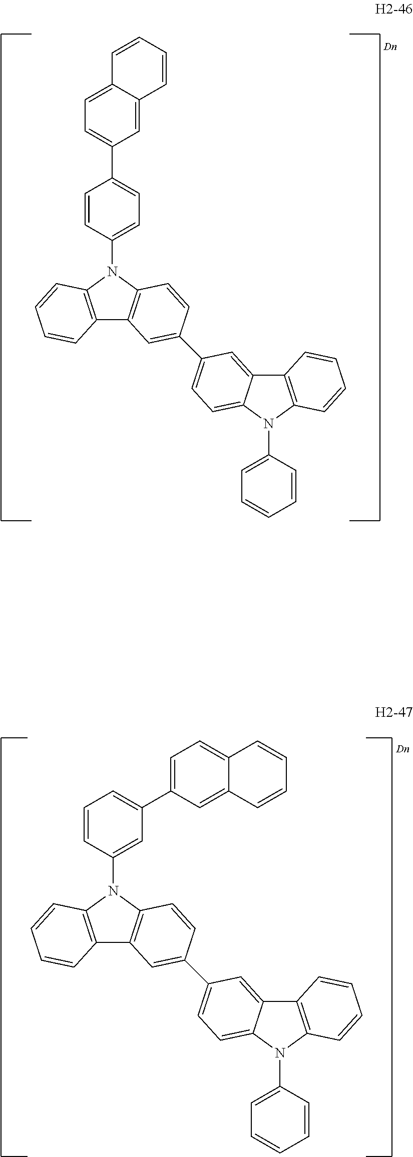

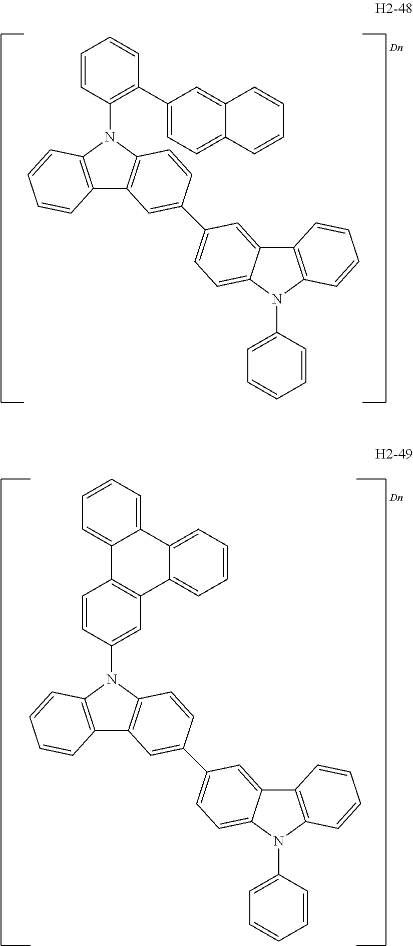

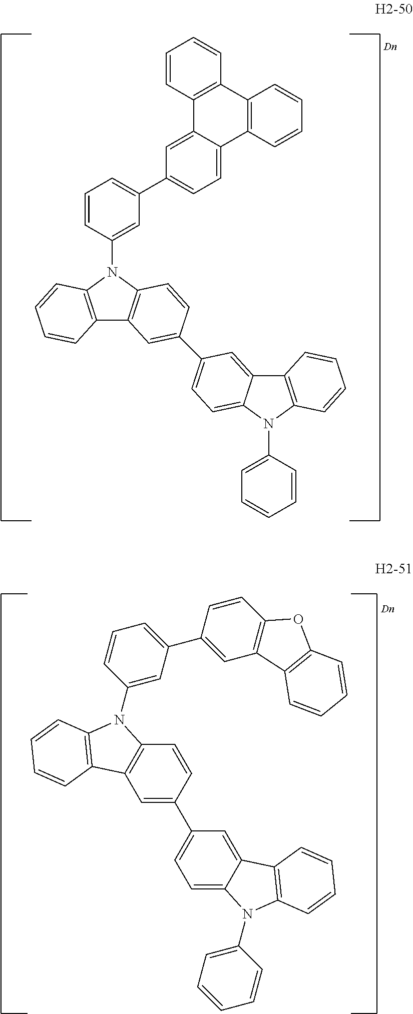

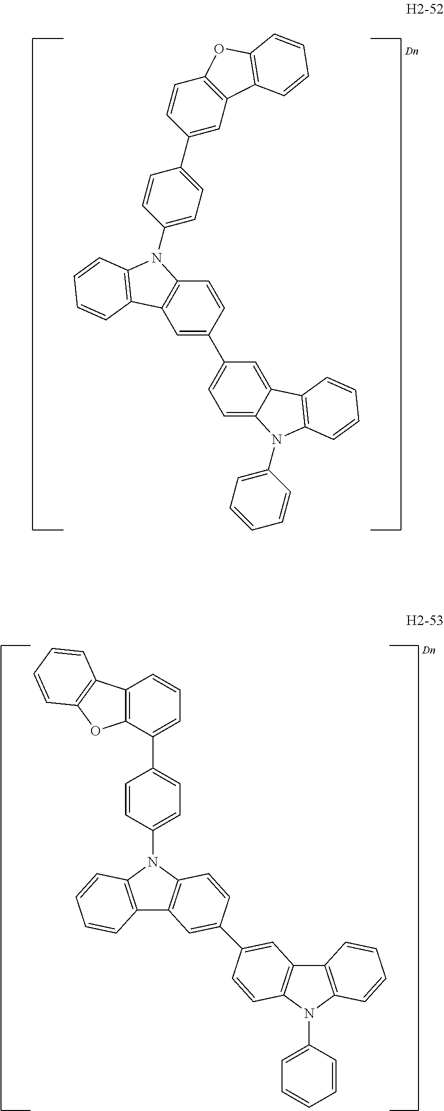

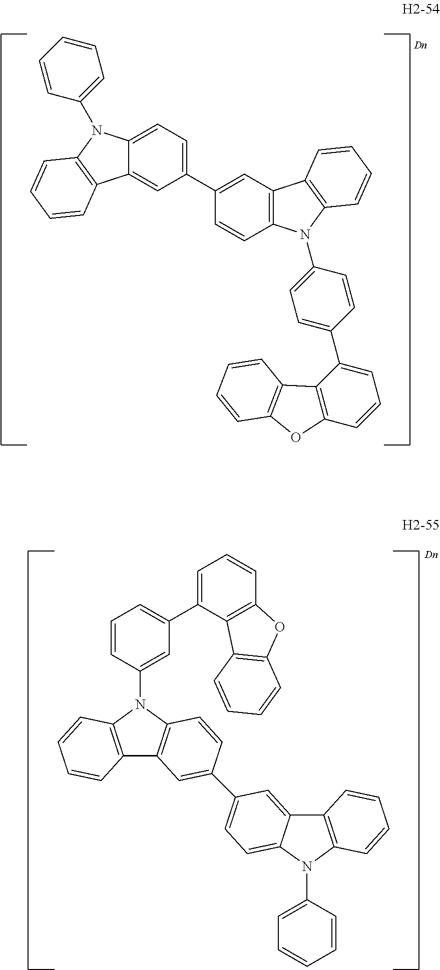

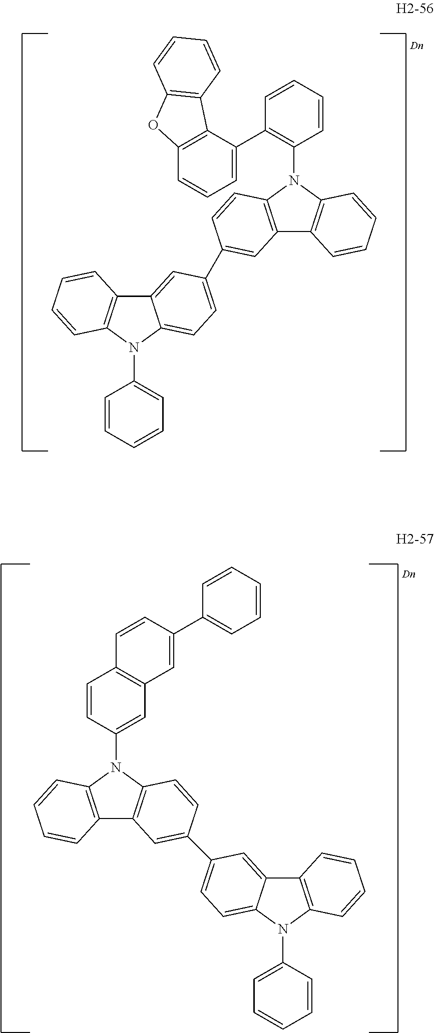

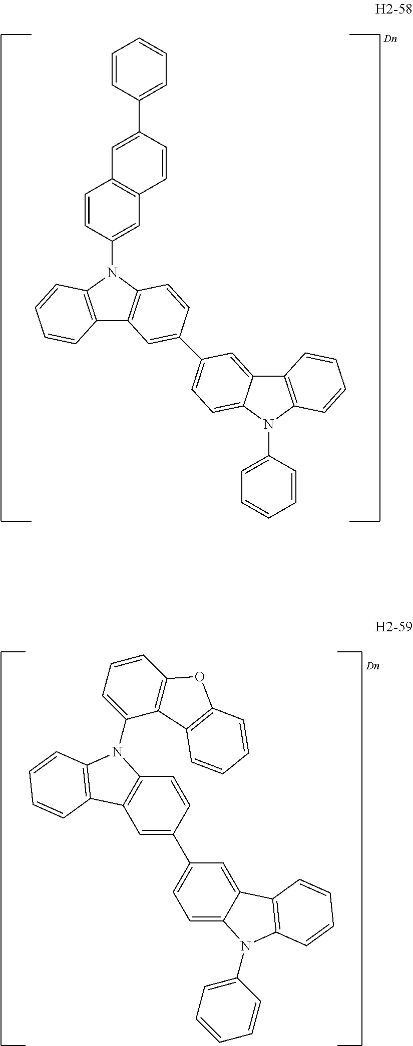

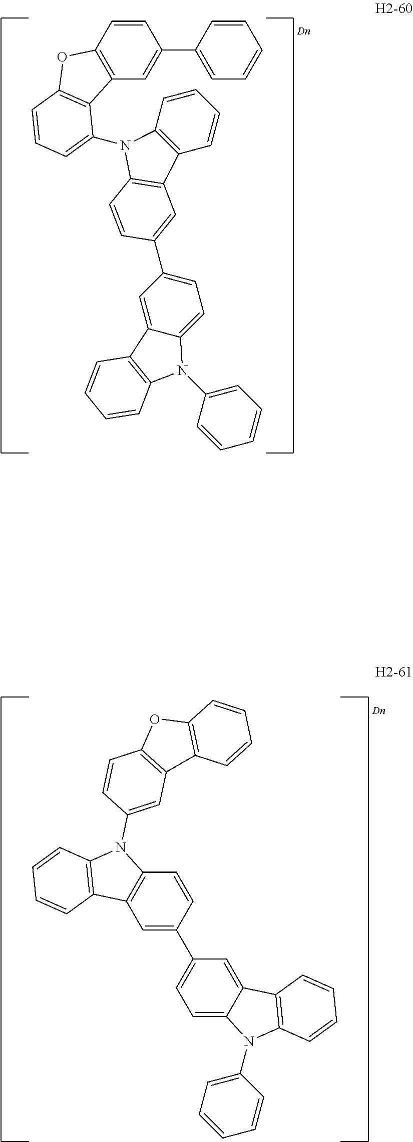

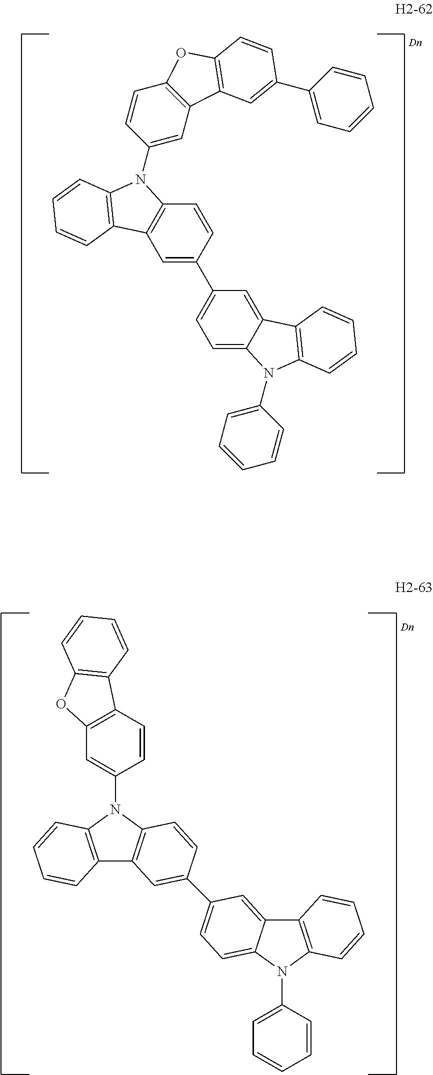

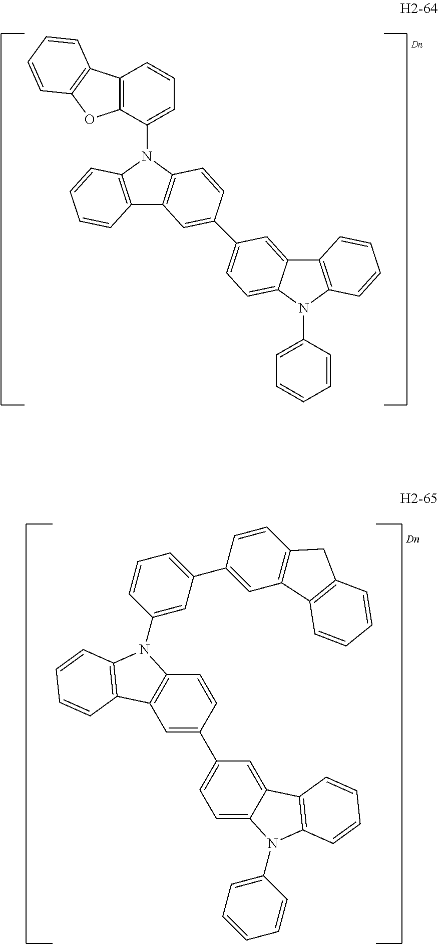

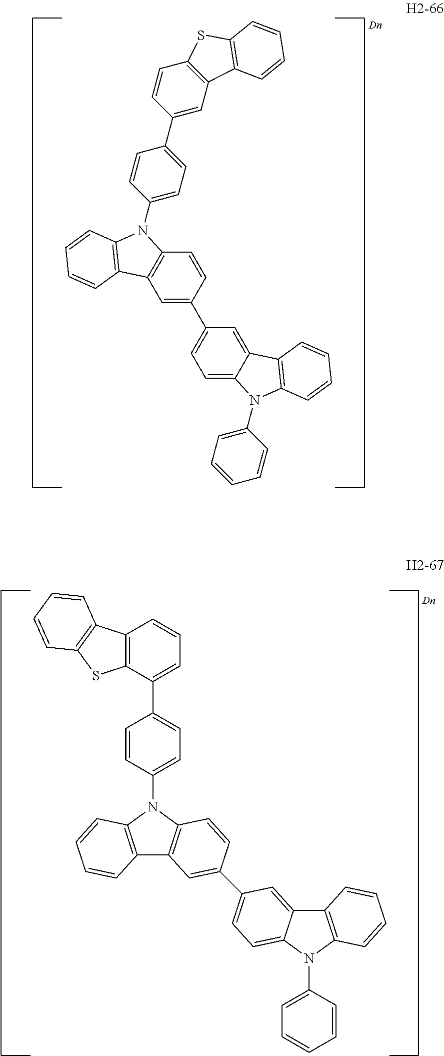

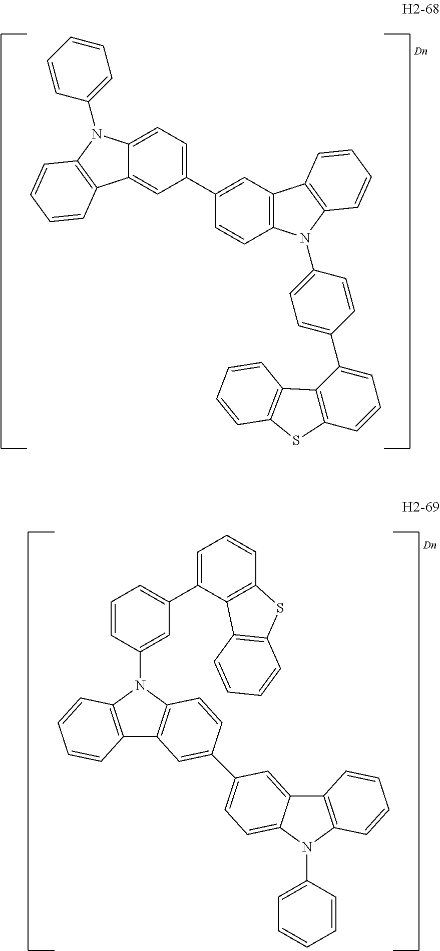

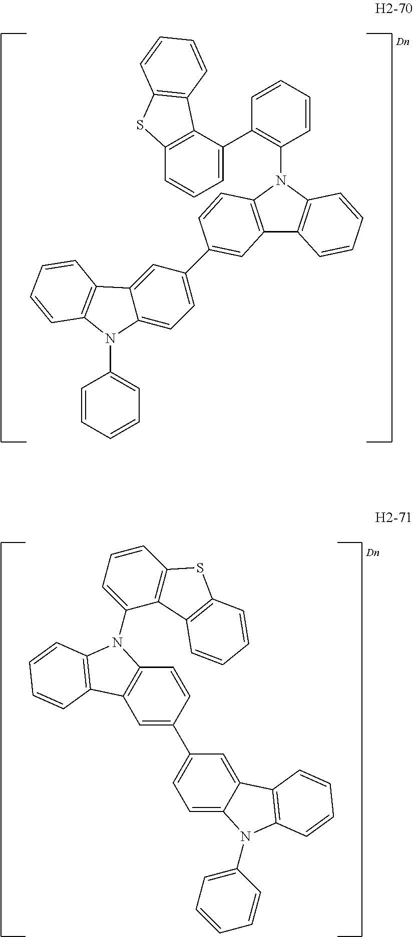

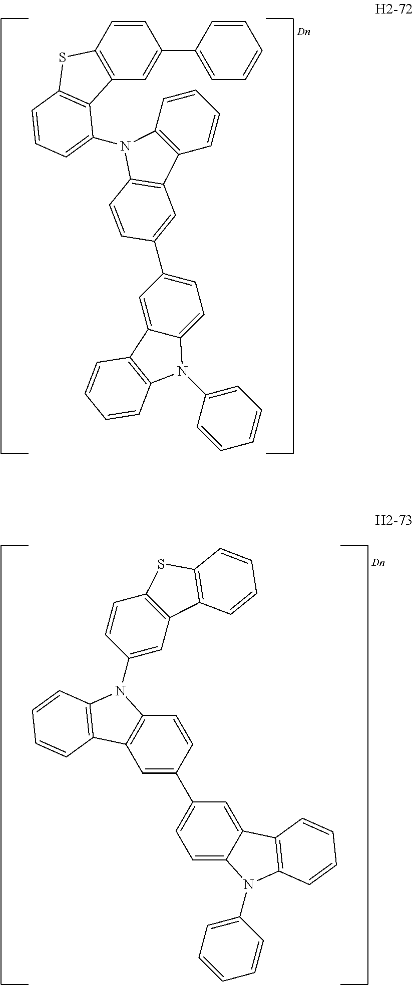

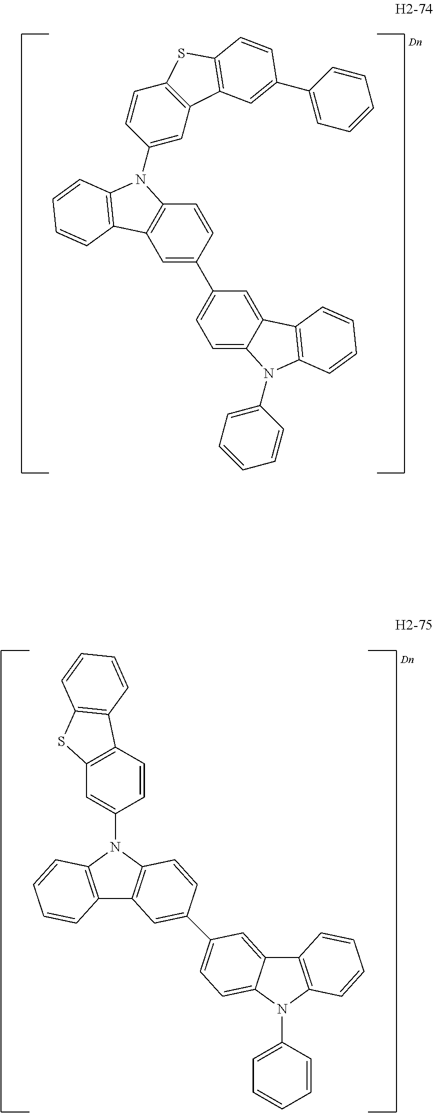

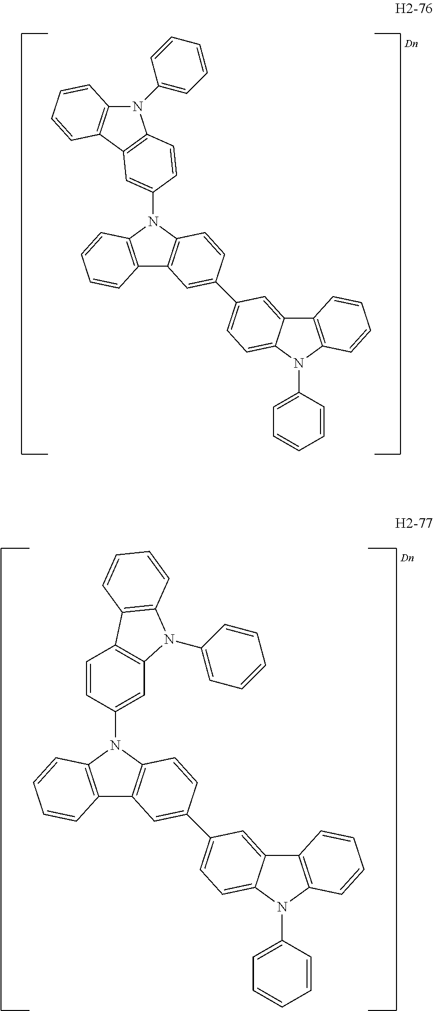

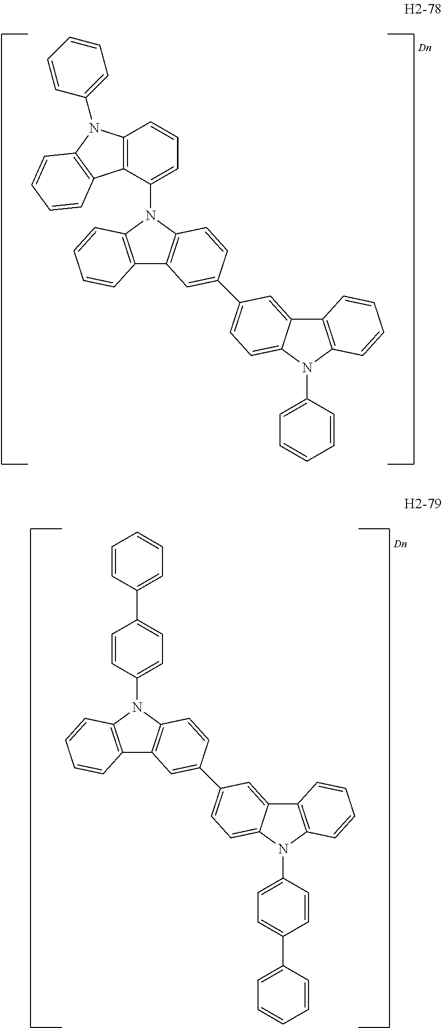

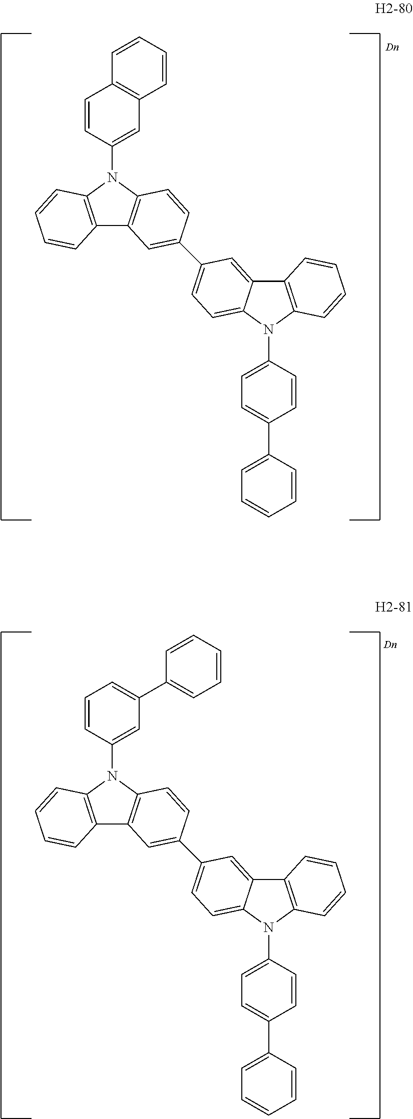

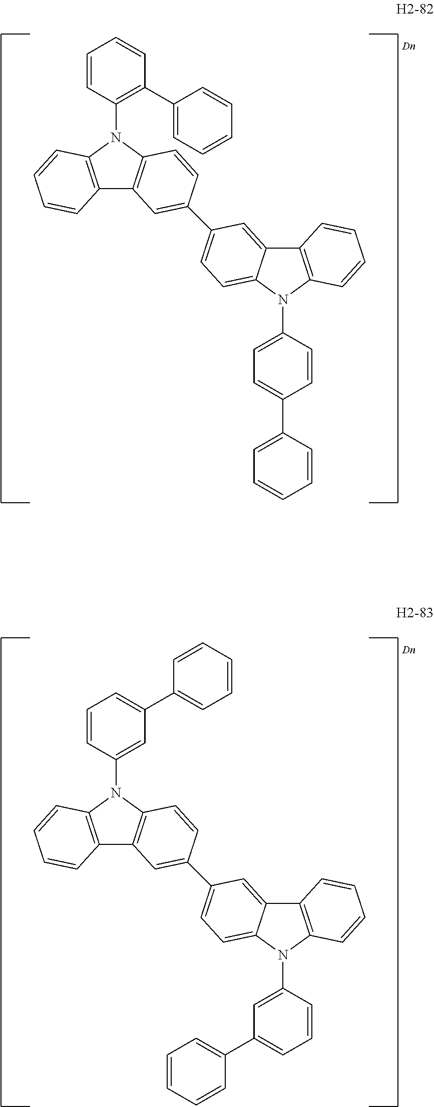

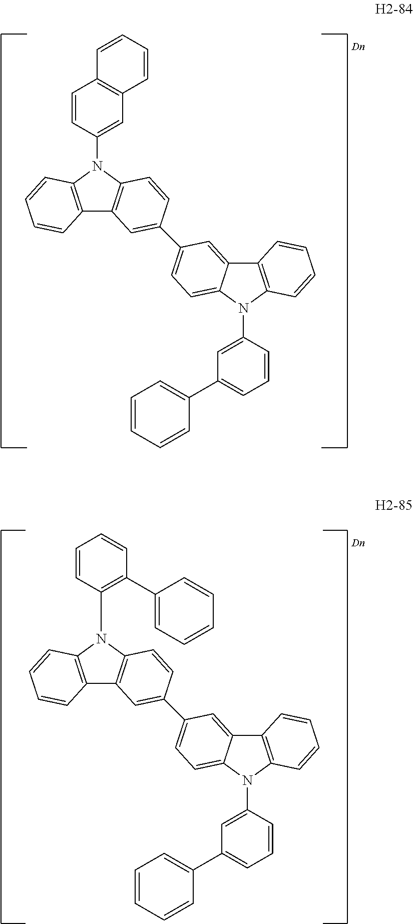

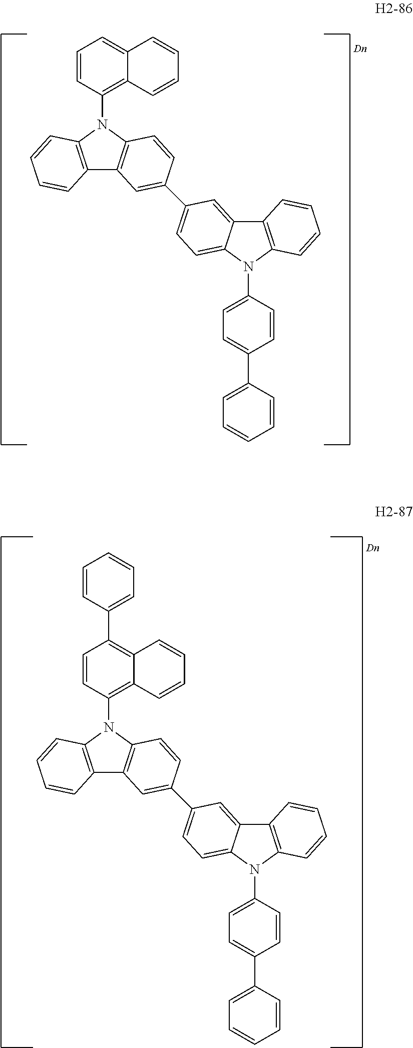

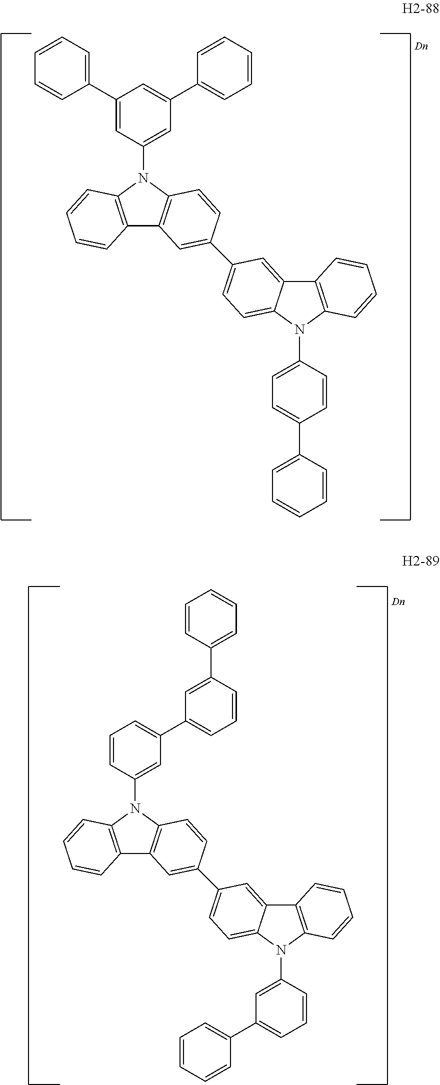

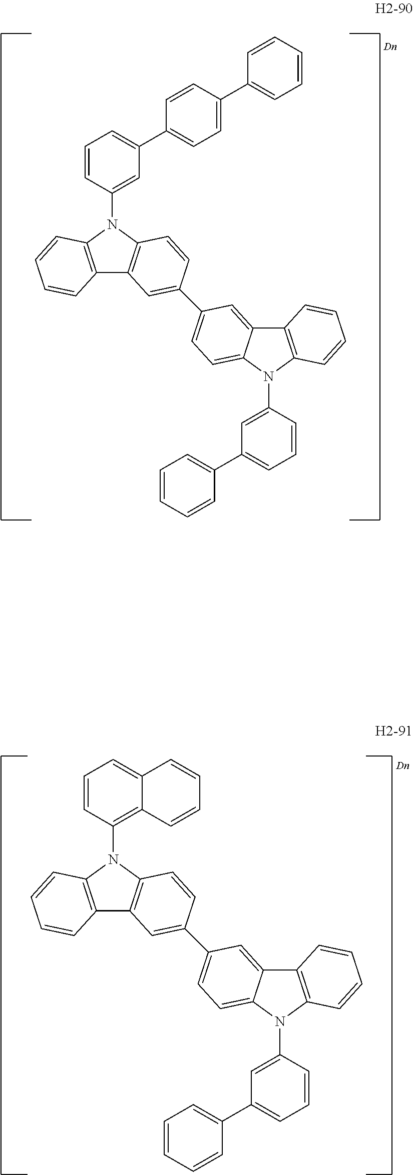

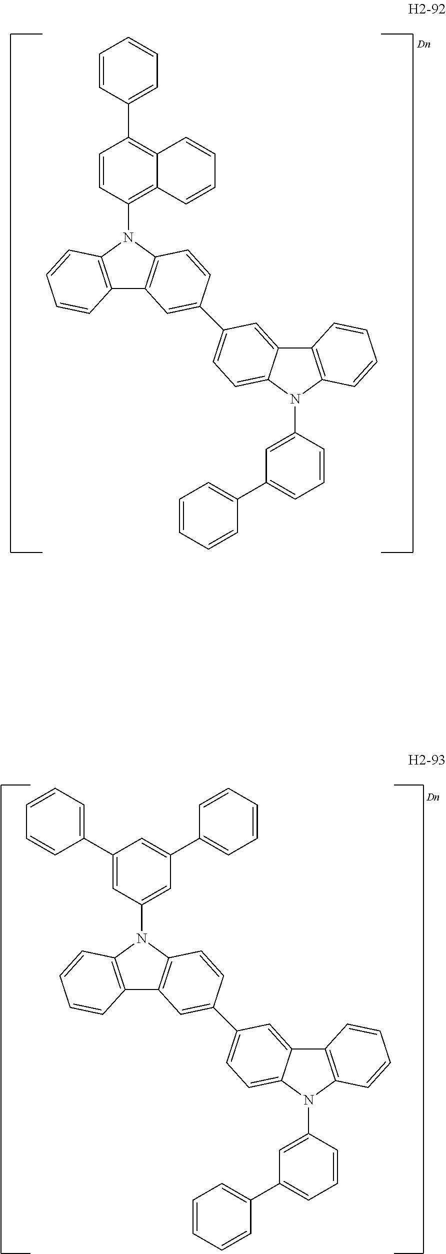

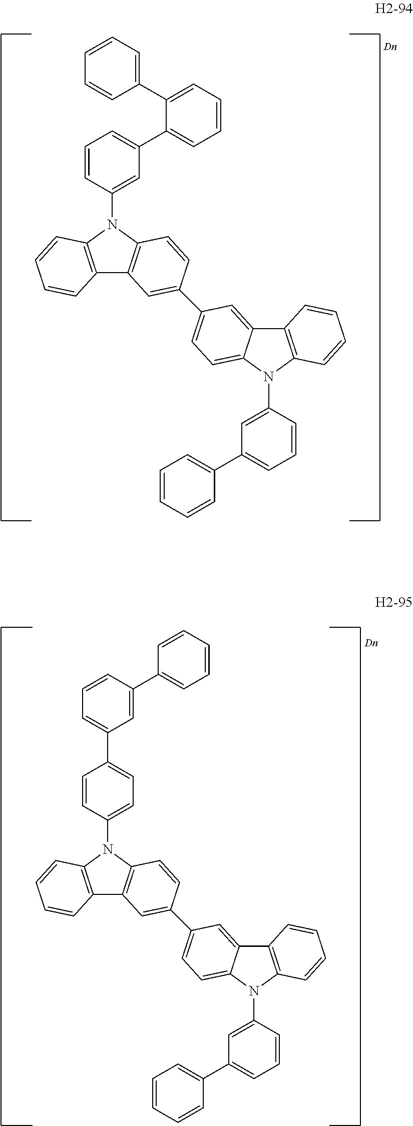

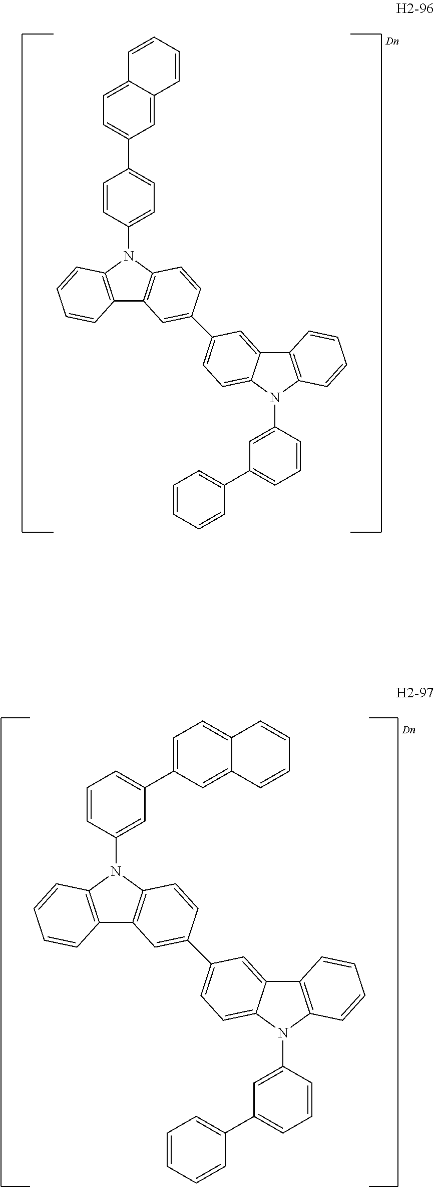

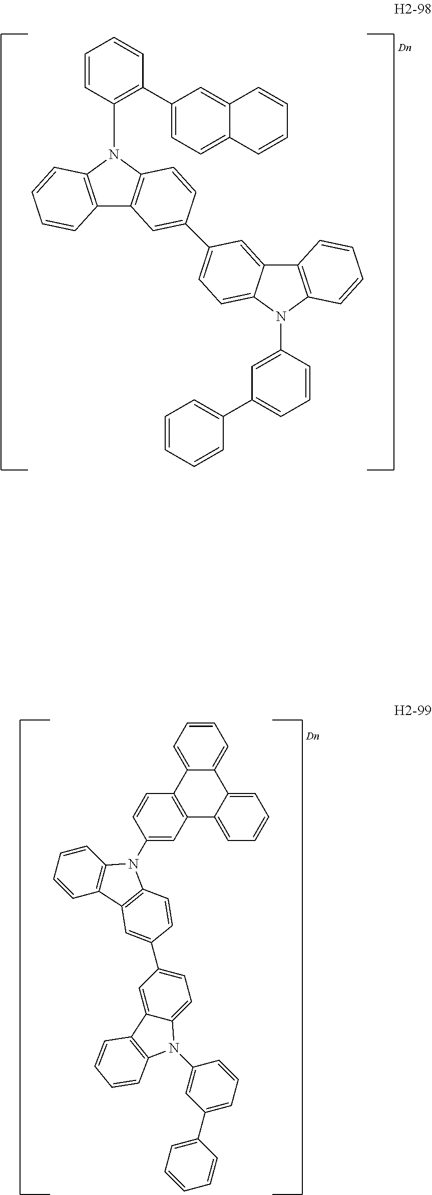

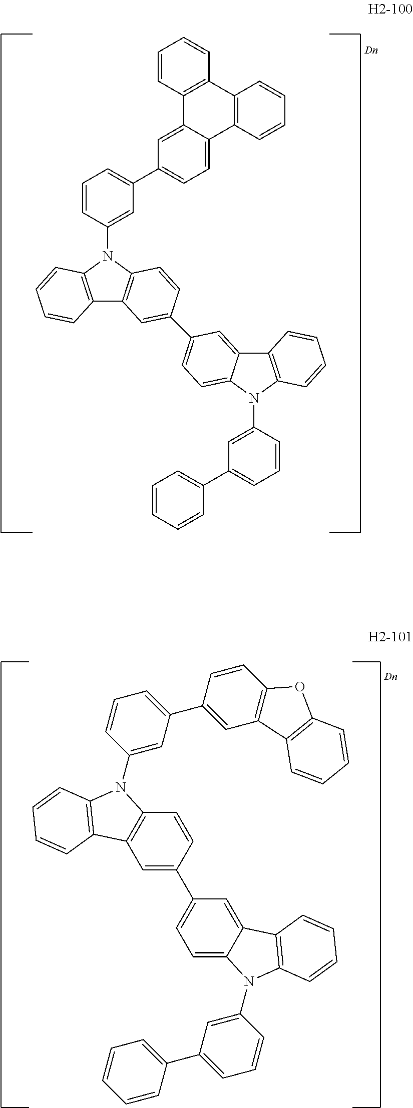

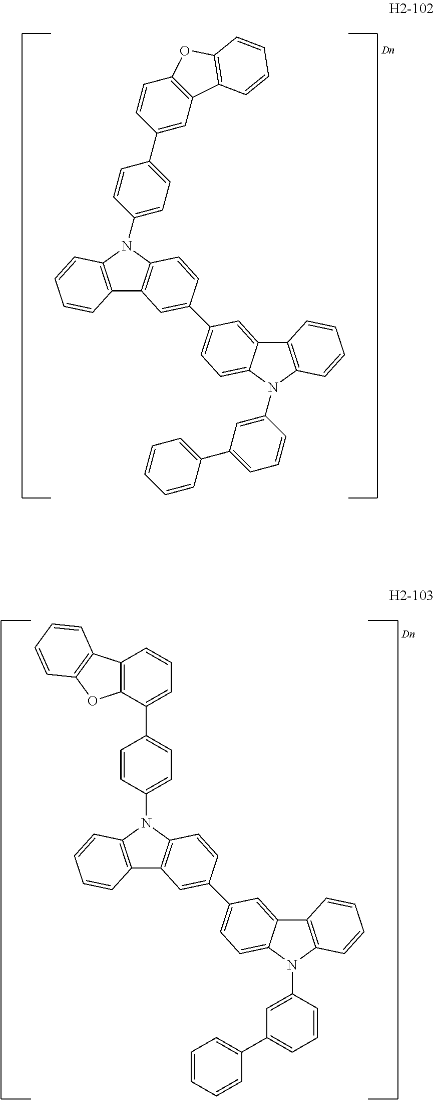

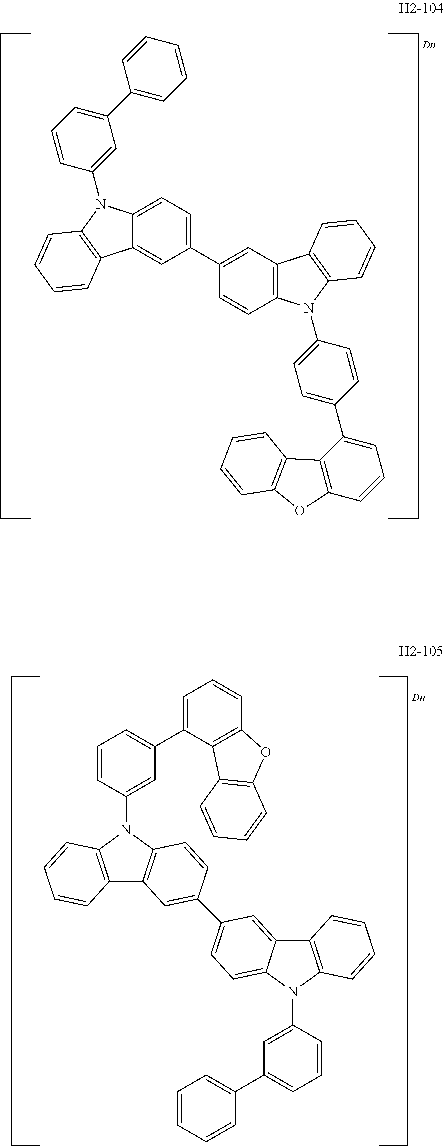

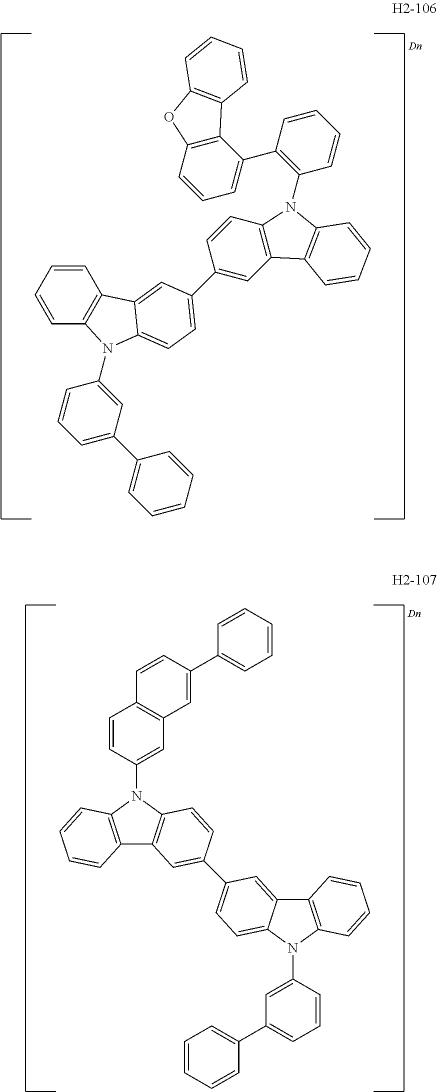

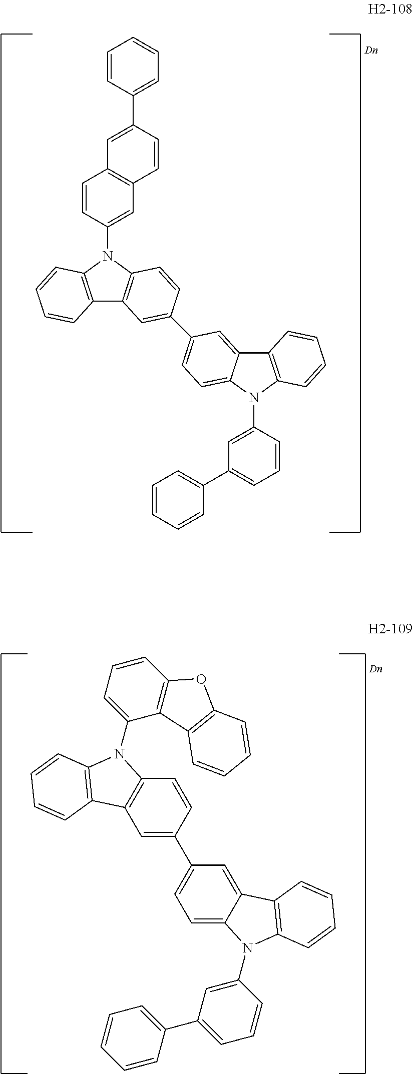

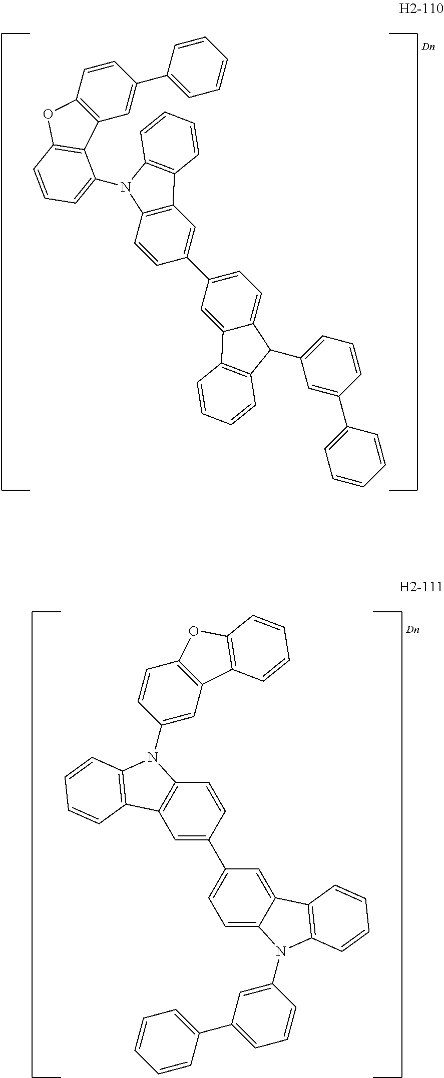

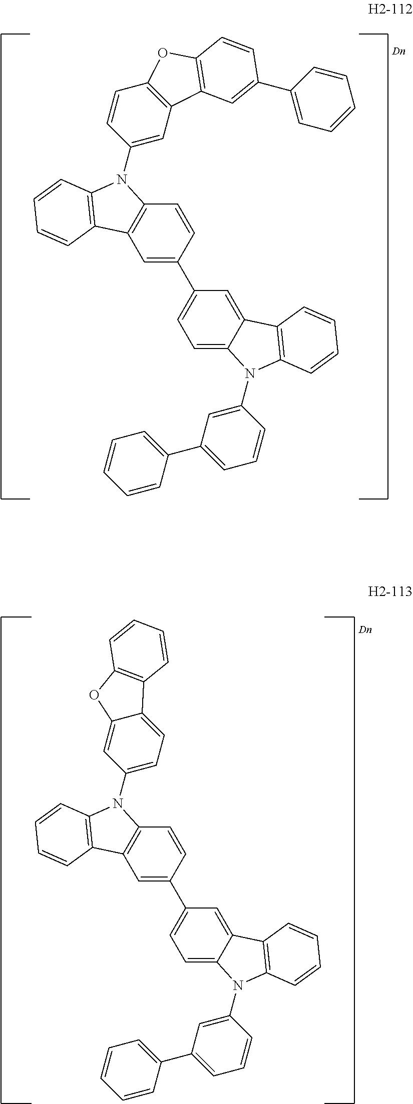

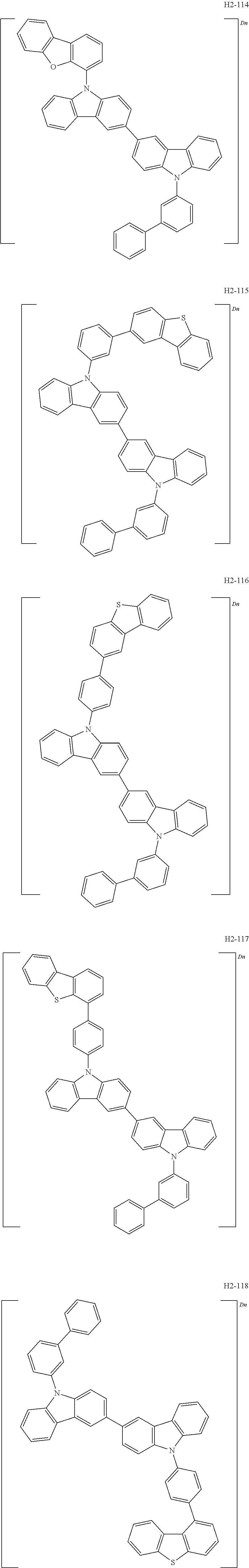

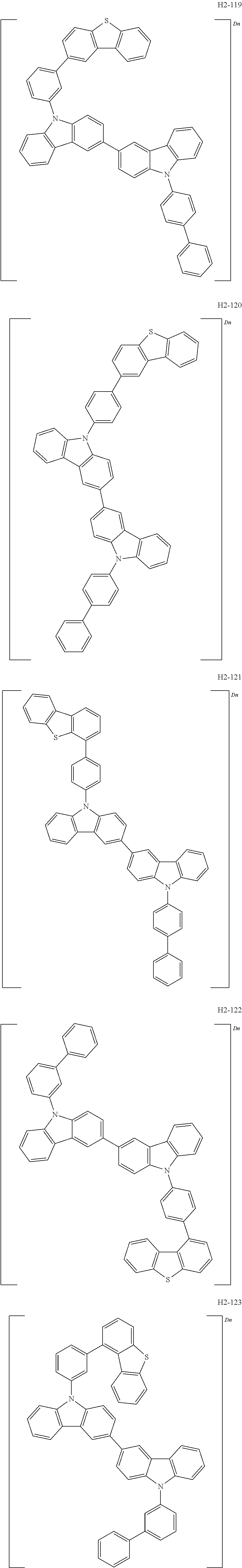

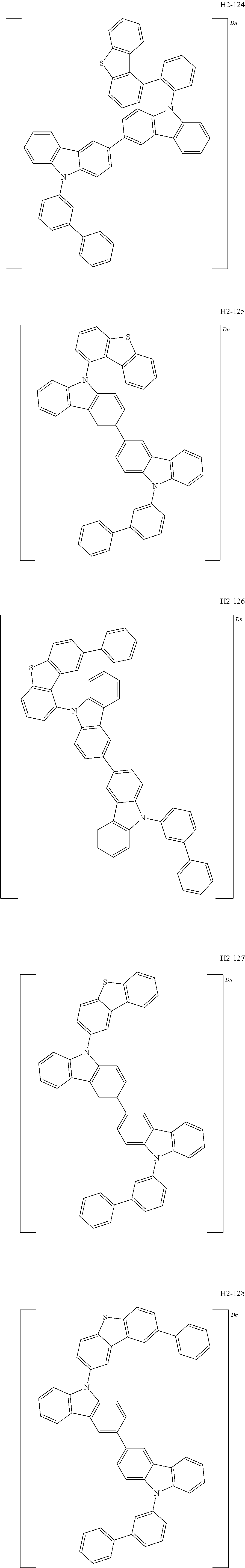

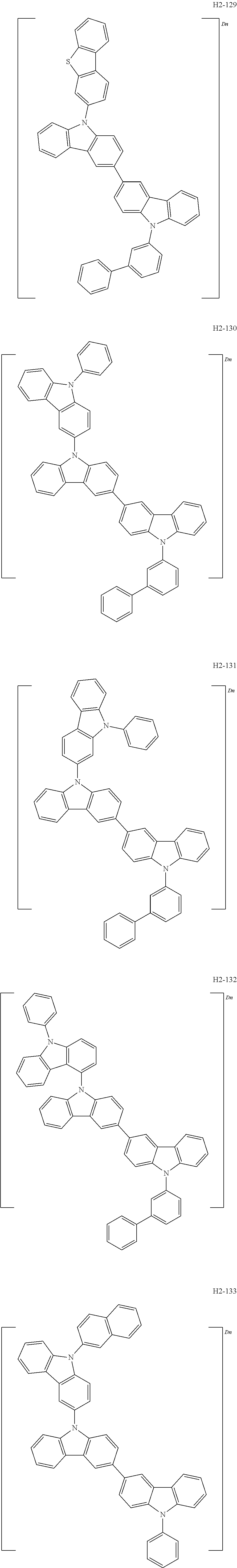

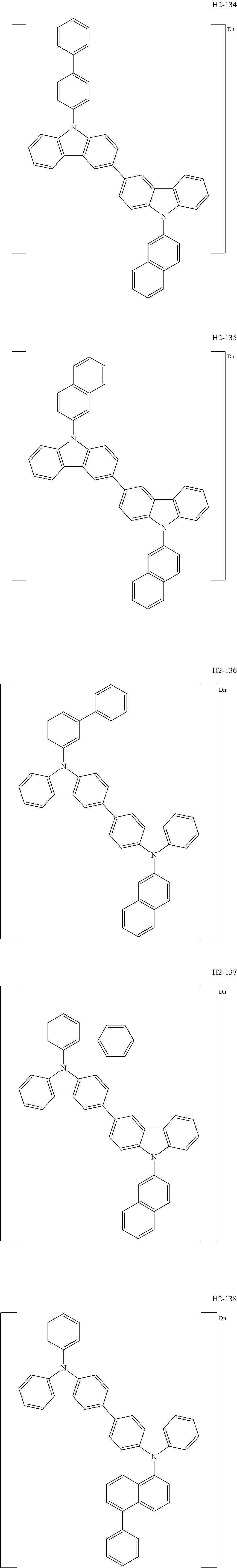

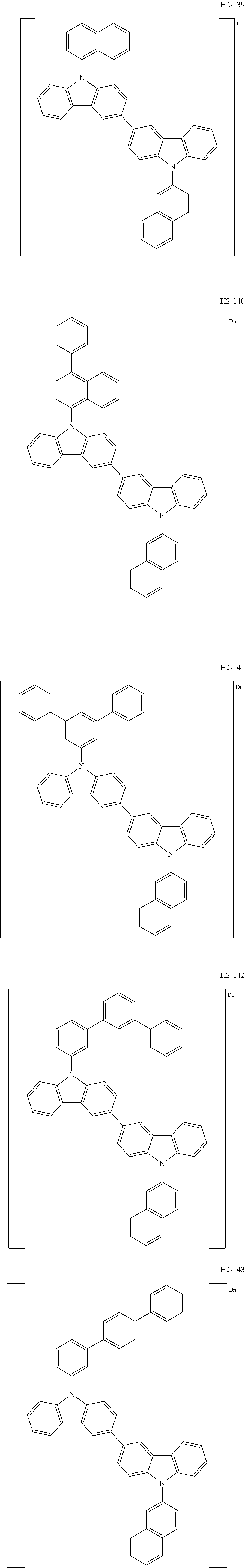

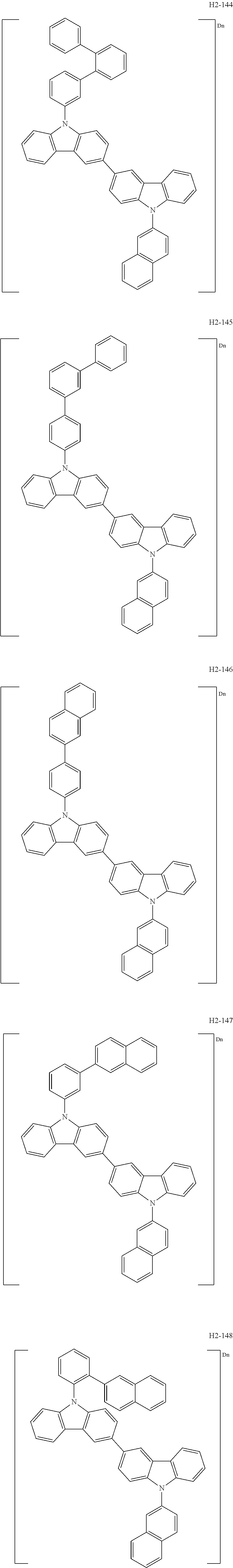

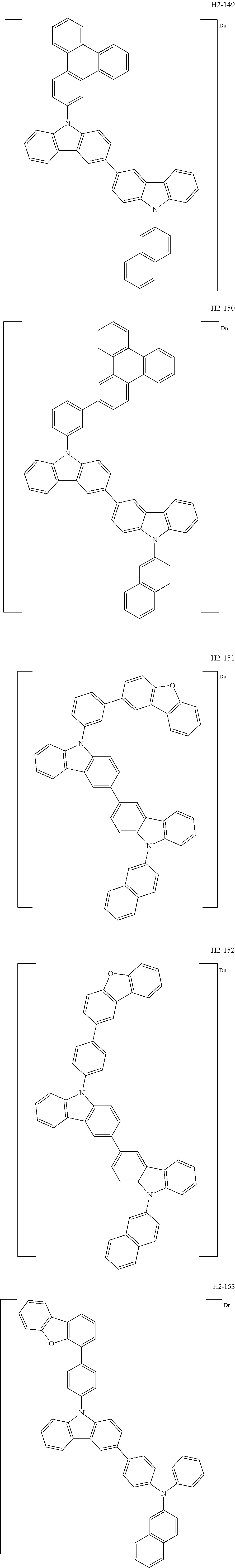

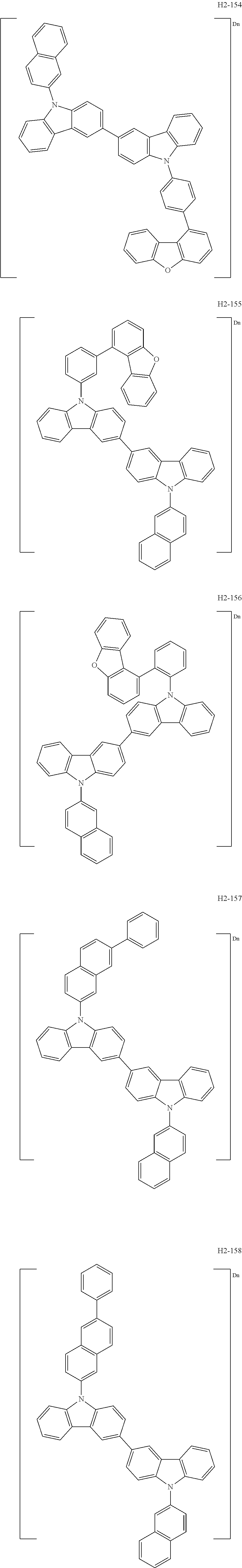

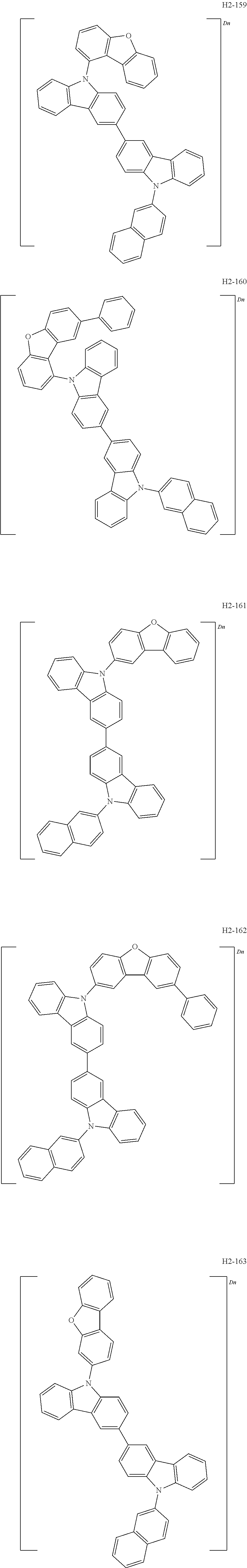

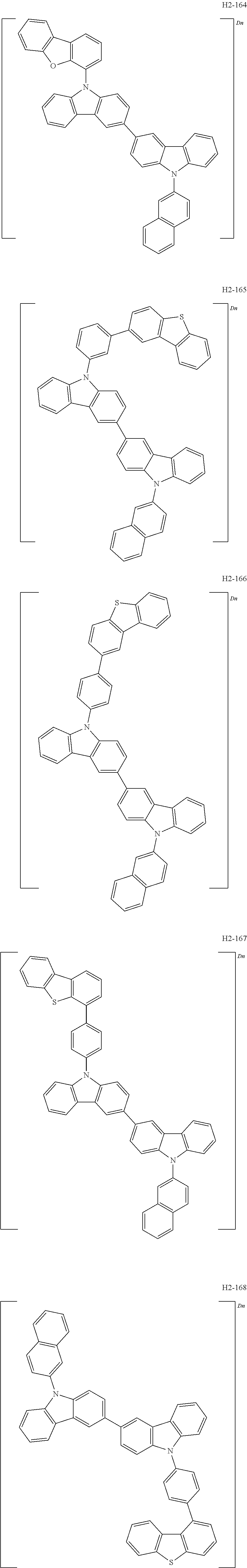

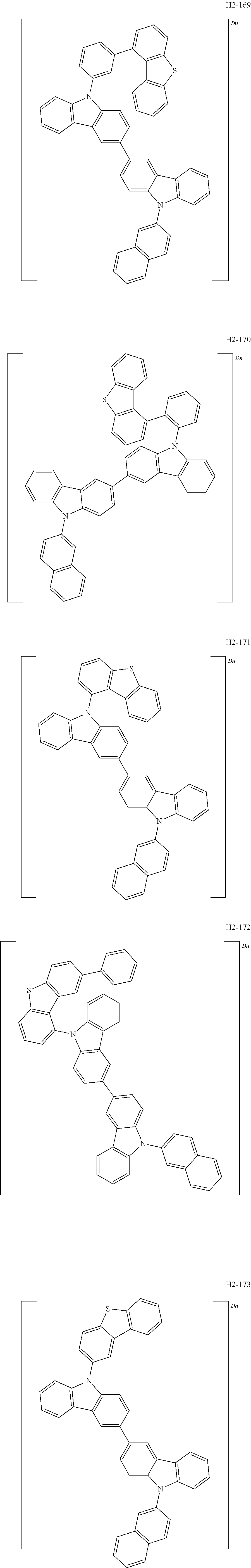

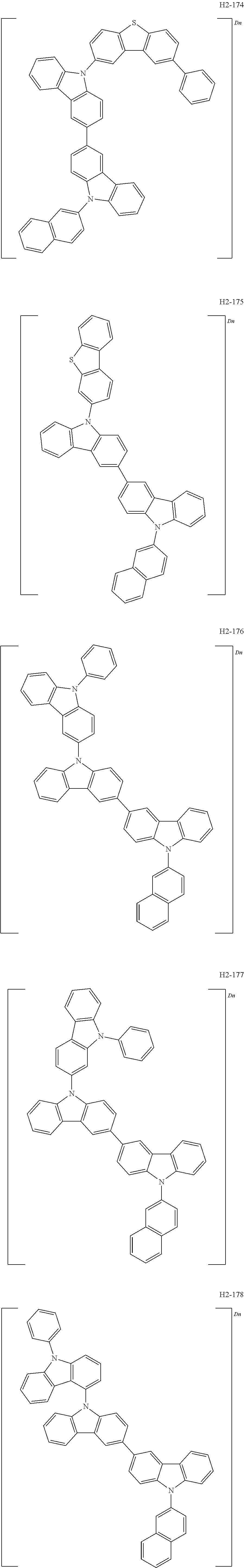

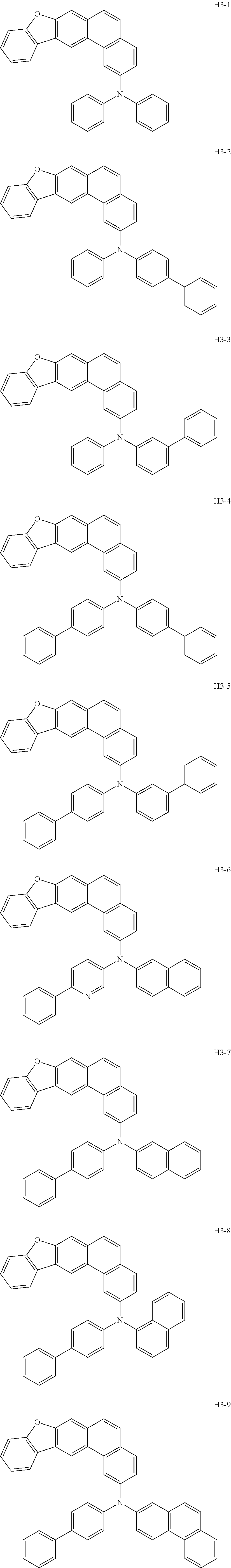

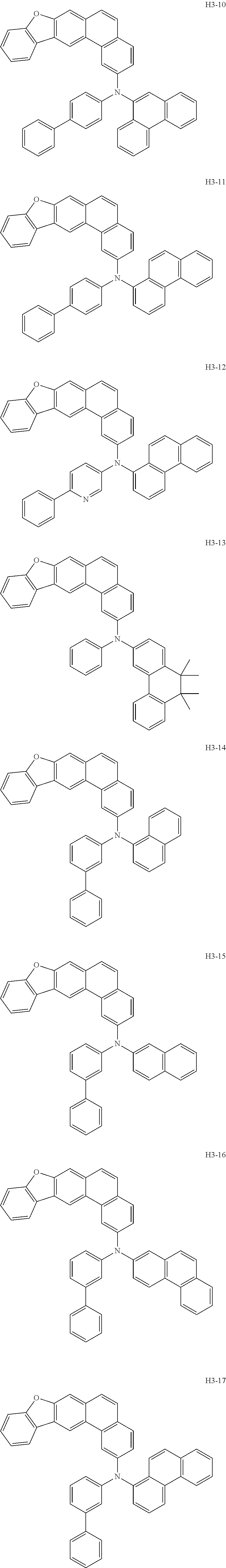

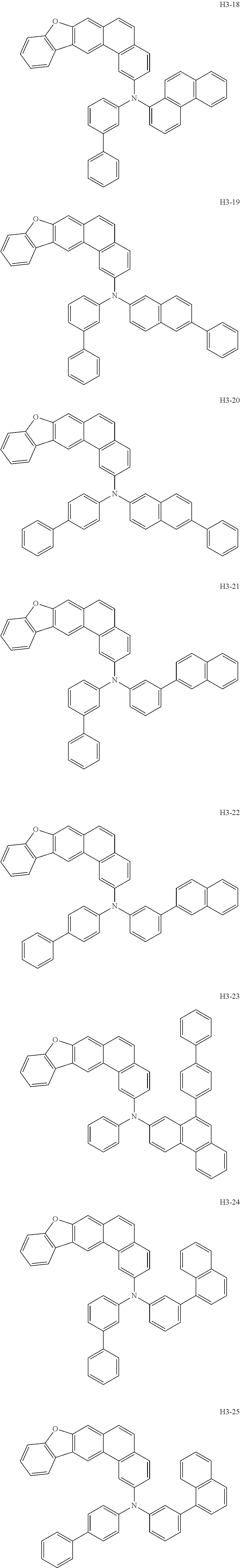

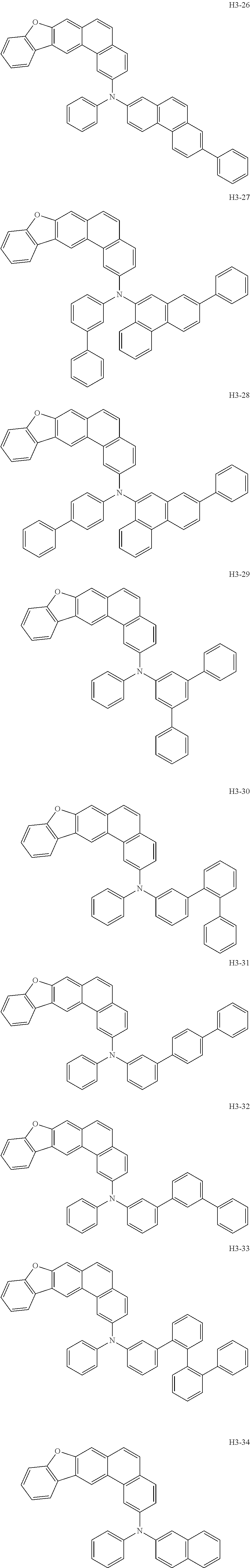

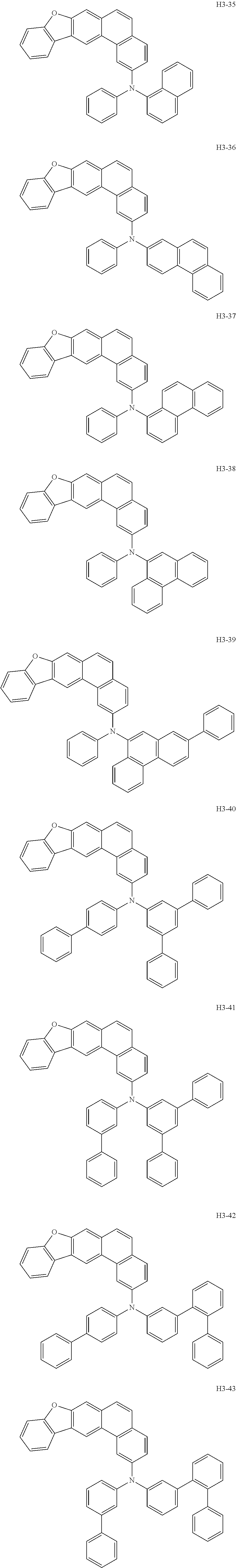

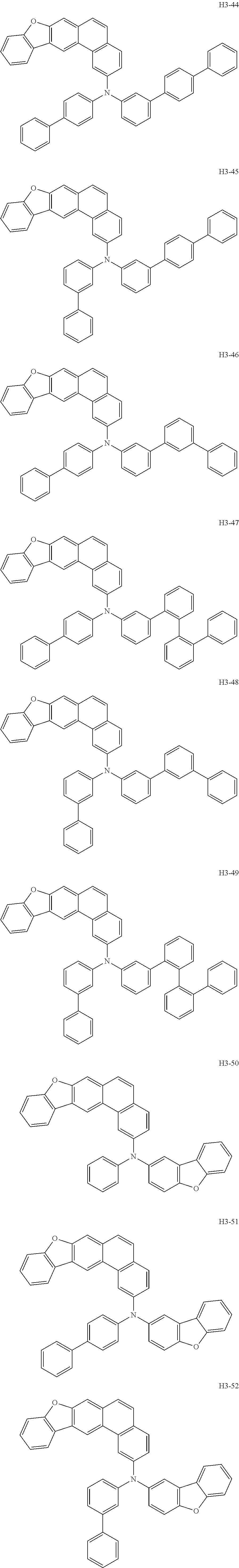

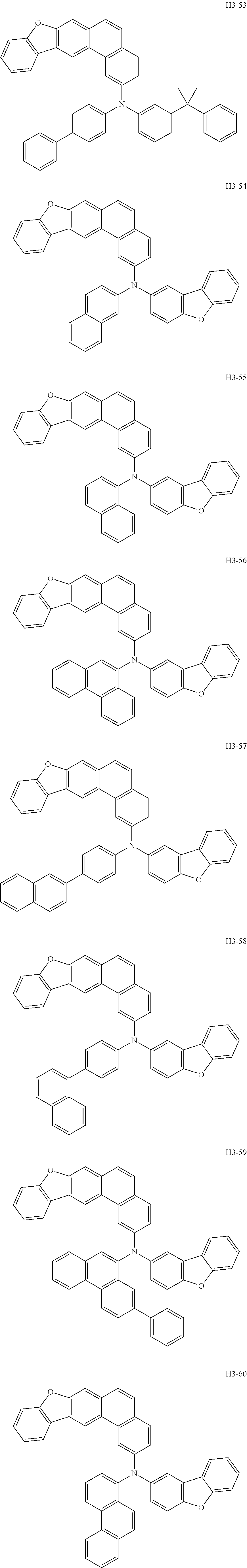

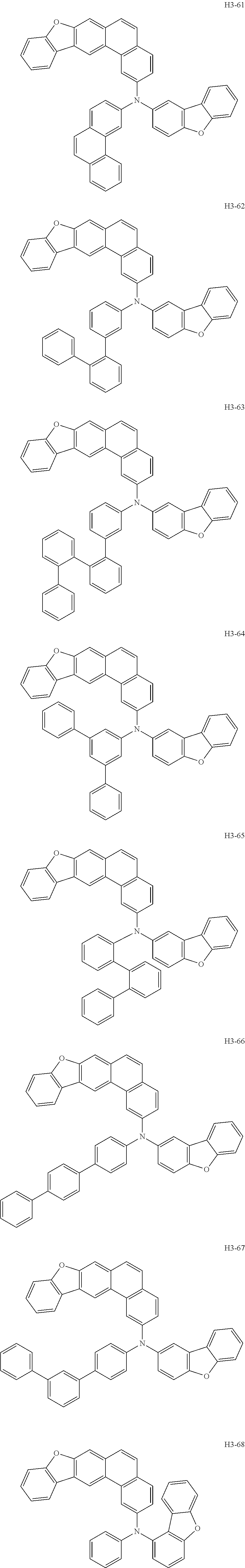

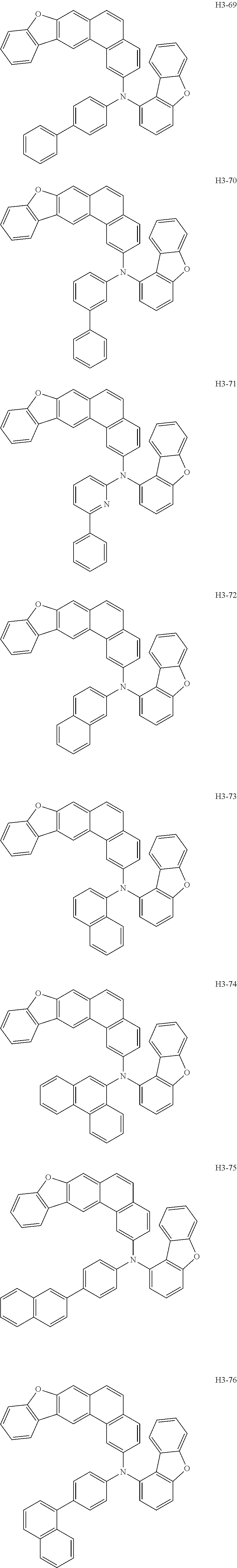

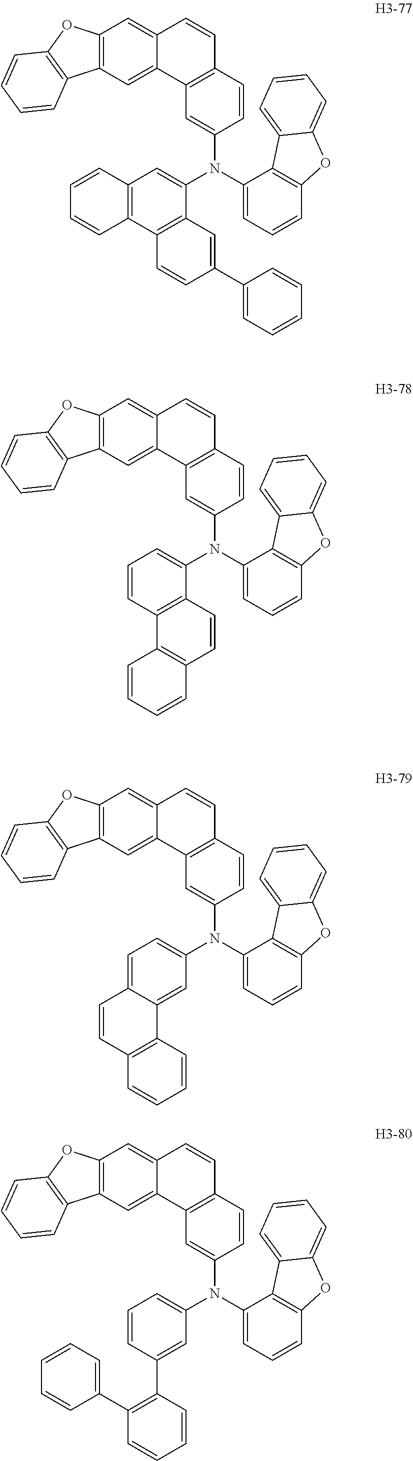 [SMILES: c1ccc(-c2ccc3c(c2)c(N(c2ccc4ccc5cc6oc7ccccc7c6cc5c4c2)c2cccc4oc5ccccc5c24)cc2ccccc23)cc1 ; c1ccc2c(c1)ccc1c(N(c3ccc4ccc5cc6oc7ccccc7c6cc5c4c3)c3cccc4oc5ccccc5c34)cccc12 ; c1ccc2c(c1)ccc1ccc(N(c3ccc4ccc5cc6oc7ccccc7c6cc5c4c3)c3cccc4oc5ccccc5c34)cc12 ; c1ccc(-c2ccccc2-c2cccc(N(c3ccc4ccc5cc6oc7ccccc7c6cc5c4c3)c3cccc4oc5ccccc5c34)c2)cc1]

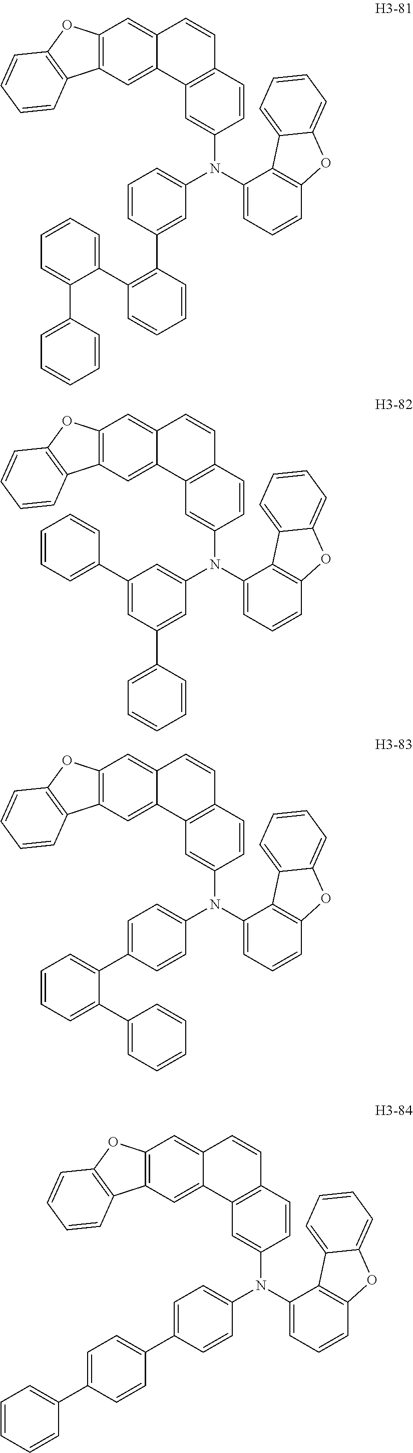

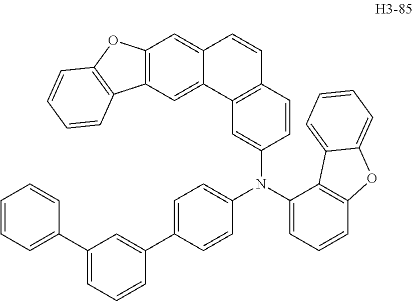 [SMILES: c1ccc(-c2cccc(-c3ccc(N(c4ccc5ccc6cc7oc8ccccc8c7cc6c5c4)c4cccc5oc6ccccc6c45)cc3)c2)cc1]

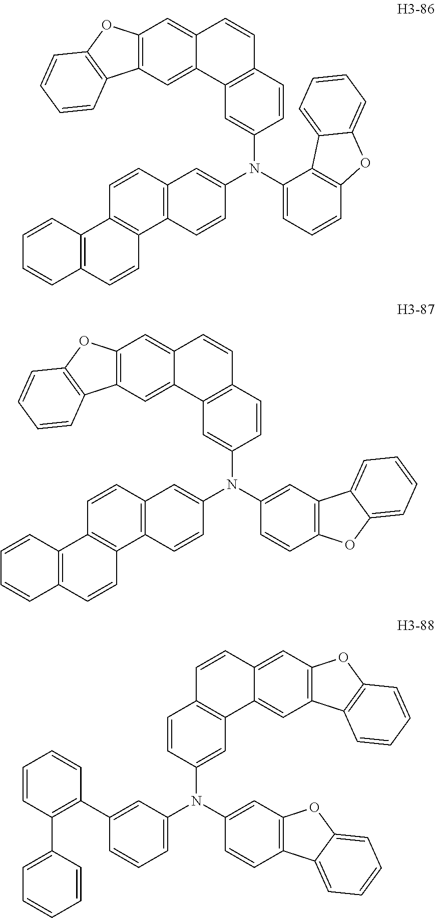 [SMILES: c1ccc2c(c1)ccc1c3ccc(N(c4ccc5ccc6cc7oc8ccccc8c7cc6c5c4)c4cccc5oc6ccccc6c45)cc3ccc21 ; c1ccc2c(c1)ccc1c3ccc(N(c4ccc5ccc6cc7oc8ccccc8c7cc6c5c4)c4ccc5oc6ccccc6c5c4)cc3ccc21 ; c1ccc(-c2ccccc2-c2cccc(N(c3ccc4c(c3)oc3ccccc34)c3ccc4ccc5cc6oc7ccccc7c6cc5c4c3)c2)cc1]

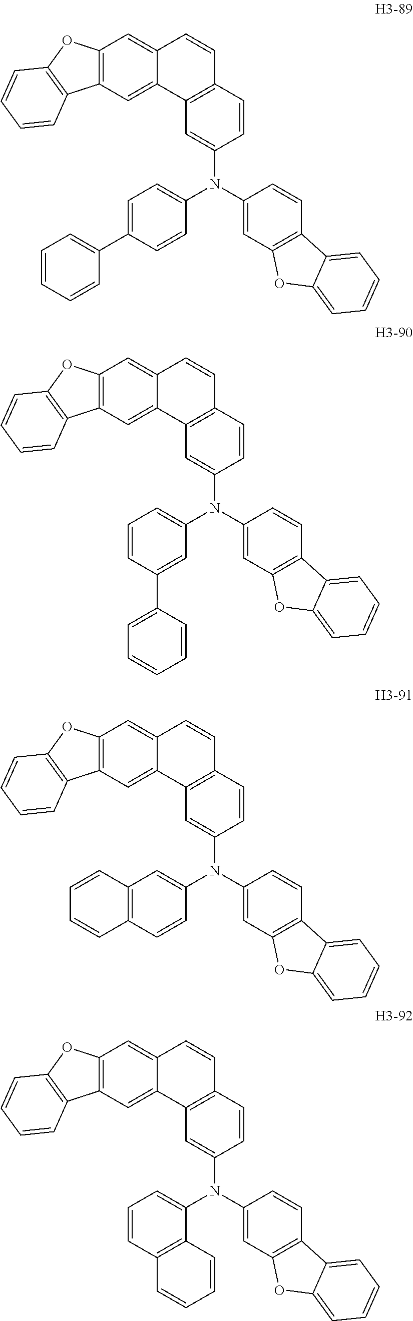 [SMILES: c1ccc(-c2ccc(N(c3ccc4c(c3)oc3ccccc34)c3ccc4ccc5cc6oc7ccccc7c6cc5c4c3)cc2)cc1 ; c1ccc(-c2cccc(N(c3ccc4c(c3)oc3ccccc34)c3ccc4ccc5cc6oc7ccccc7c6cc5c4c3)c2)cc1 ; c1ccc2cc(N(c3ccc4c(c3)oc3ccccc34)c3ccc4ccc5cc6oc7ccccc7c6cc5c4c3)ccc2c1 ; c1ccc2c(N(c3ccc4c(c3)oc3ccccc34)c3ccc4ccc5cc6oc7ccccc7c6cc5c4c3)cccc2c1]

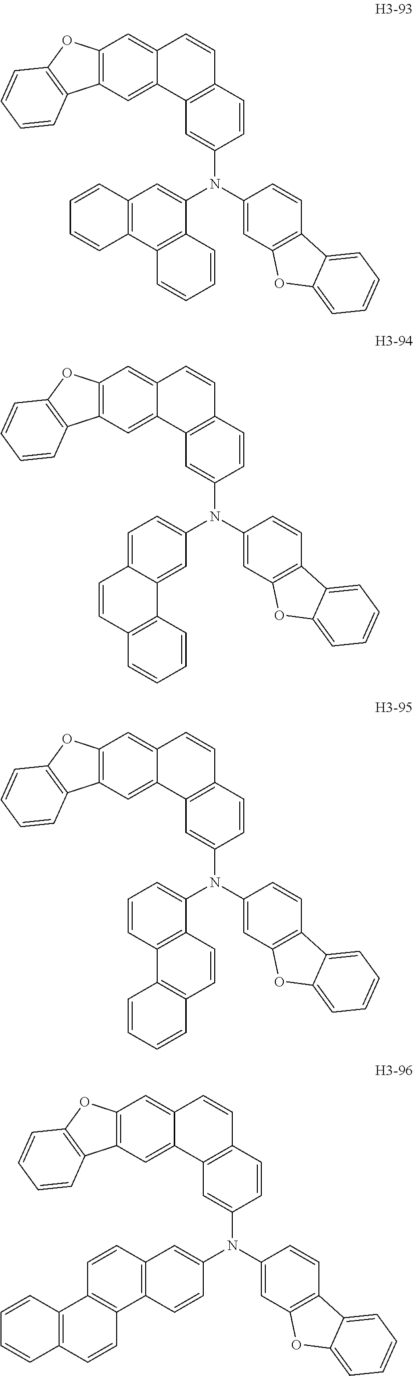 [SMILES: c1ccc2c(c1)cc(N(c1ccc3c(c1)oc1ccccc13)c1ccc3ccc4cc5oc6ccccc6c5cc4c3c1)c1ccccc12 ; c1ccc2c(c1)ccc1ccc(N(c3ccc4c(c3)oc3ccccc34)c3ccc4ccc5cc6oc7ccccc7c6cc5c4c3)cc12 ; c1ccc2c(c1)ccc1c(N(c3ccc4c(c3)oc3ccccc34)c3ccc4ccc5cc6oc7ccccc7c6cc5c4c3)cccc12 ; c1ccc2c(c1)ccc1c3ccc(N(c4ccc5c(c4)oc4ccccc45)c4ccc5ccc6cc7oc8ccccc8c7cc6c5c4)cc3ccc21]

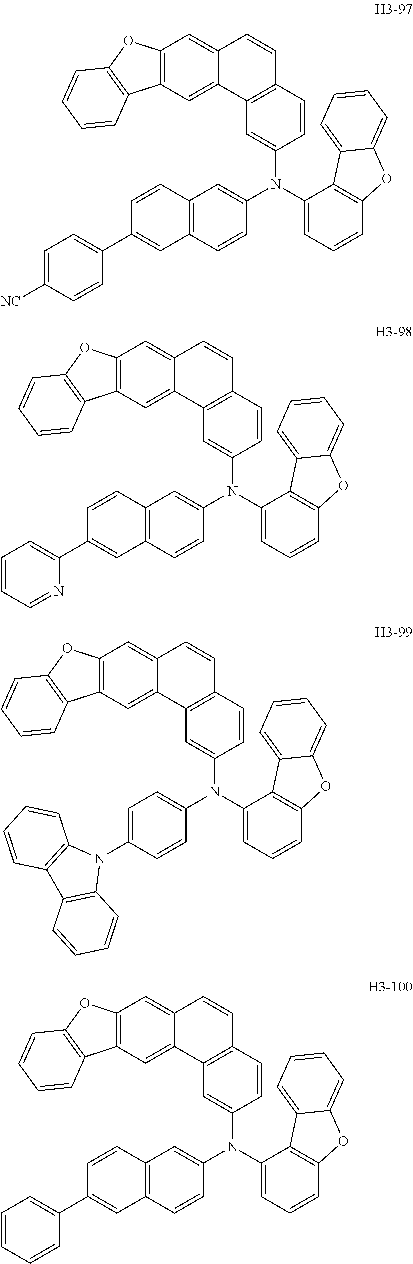 [SMILES: N#Cc1ccc(-c2ccc3cc(N(c4ccc5ccc6cc7oc8ccccc8c7cc6c5c4)c4cccc5oc6ccccc6c45)ccc3c2)cc1 ; c1ccc(-c2ccc3cc(N(c4ccc5ccc6cc7oc8ccccc8c7cc6c5c4)c4cccc5oc6ccccc6c45)ccc3c2)nc1 ; c1ccc2c(c1)oc1cc3ccc4ccc(N(c5ccc(-n6c7ccccc7c7ccccc76)cc5)c5cccc6oc7ccccc7c56)cc4c3cc12 ; c1ccc(-c2ccc3cc(N(c4ccc5ccc6cc7oc8ccccc8c7cc6c5c4)c4cccc5oc6ccccc6c45)ccc3c2)cc1]

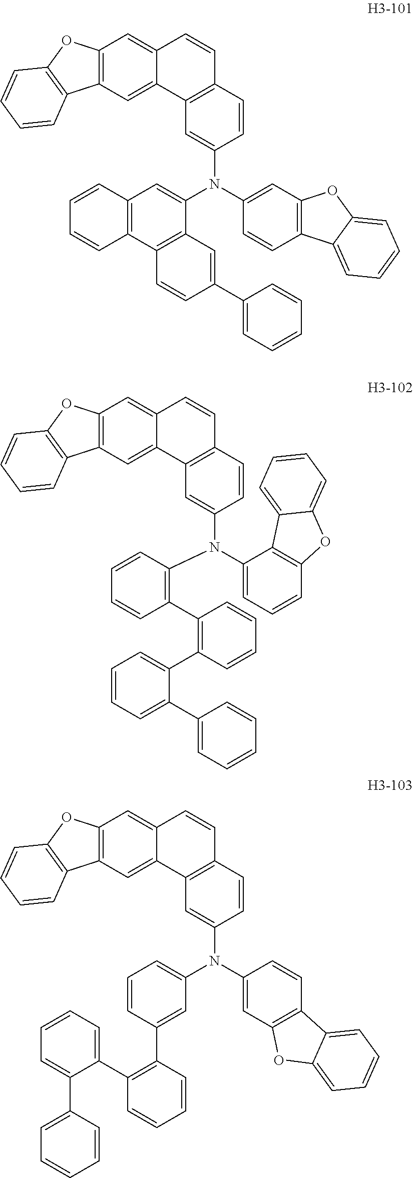 [SMILES: c1ccc(-c2ccc3c(c2)c(N(c2ccc4c(c2)oc2ccccc24)c2ccc4ccc5cc6oc7ccccc7c6cc5c4c2)cc2ccccc23)cc1 ; c1ccc(-c2ccccc2-c2ccccc2-c2ccccc2N(c2ccc3ccc4cc5oc6ccccc6c5cc4c3c2)c2cccc3oc4ccccc4c23)cc1 ; c1ccc(-c2ccccc2-c2ccccc2-c2cccc(N(c3ccc4c(c3)oc3ccccc34)c3ccc4ccc5cc6oc7ccccc7c6cc5c4c3)c2)cc1]

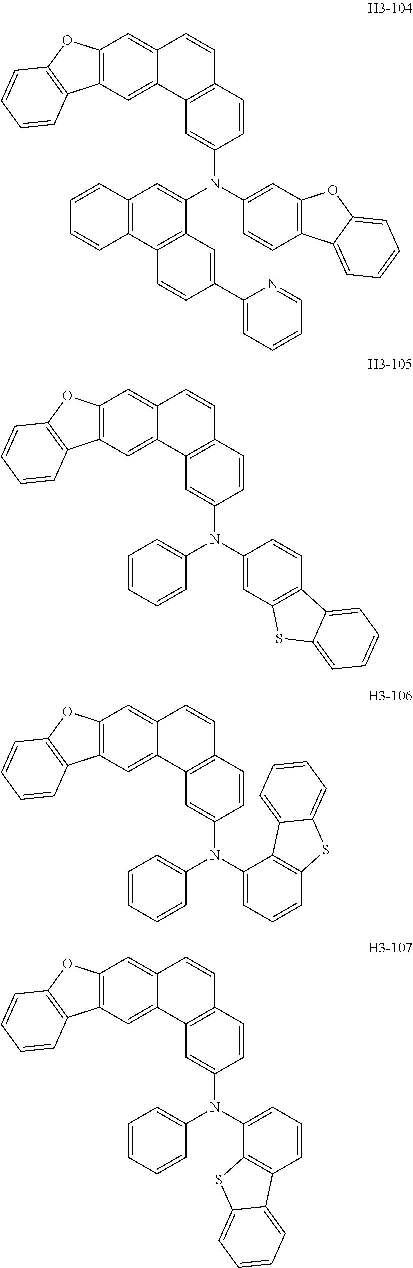

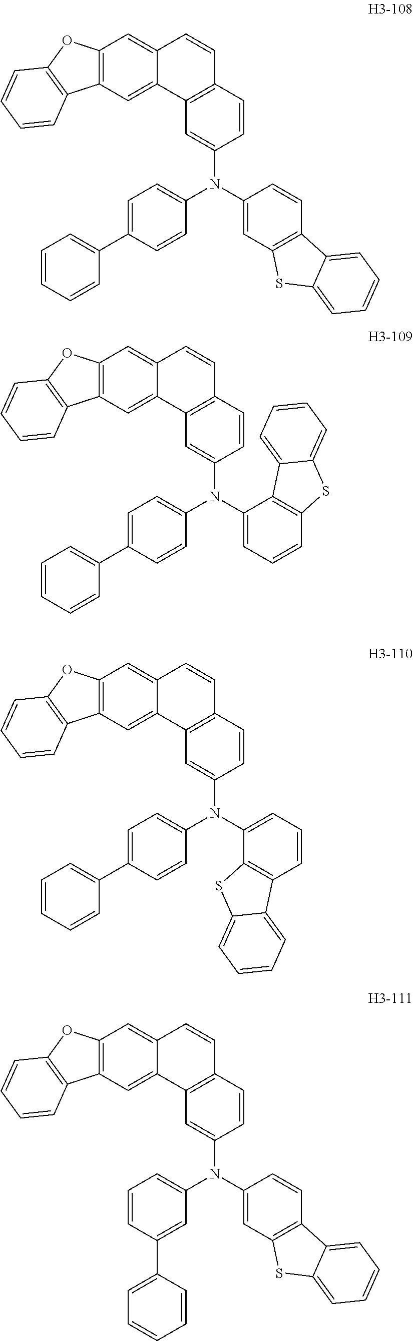 [SMILES: c1ccc(-c2ccc(N(c3ccc4c(c3)sc3ccccc34)c3ccc4ccc5cc6oc7ccccc7c6cc5c4c3)cc2)cc1 ; c1ccc(-c2ccc(N(c3ccc4ccc5cc6oc7ccccc7c6cc5c4c3)c3cccc4sc5ccccc5c34)cc2)cc1 ; c1ccc(-c2ccc(N(c3ccc4ccc5cc6oc7ccccc7c6cc5c4c3)c3cccc4c3sc3ccccc34)cc2)cc1 ; c1ccc(-c2cccc(N(c3ccc4c(c3)sc3ccccc34)c3ccc4ccc5cc6oc7ccccc7c6cc5c4c3)c2)cc1]

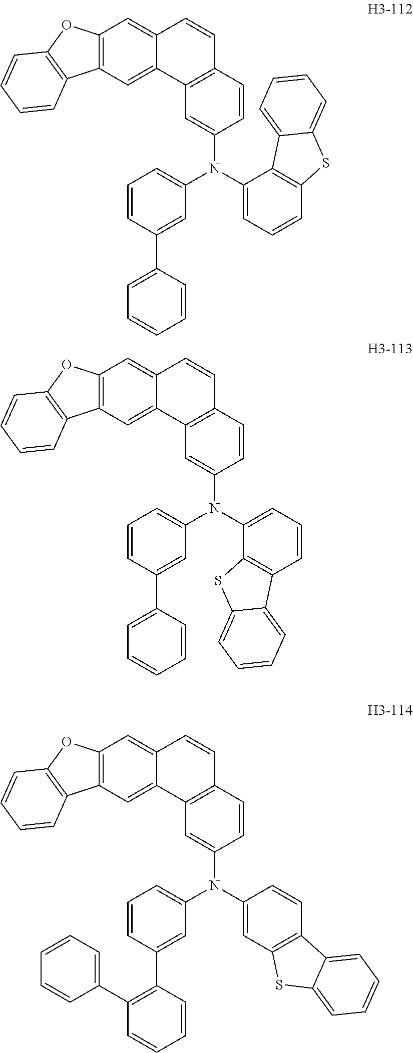

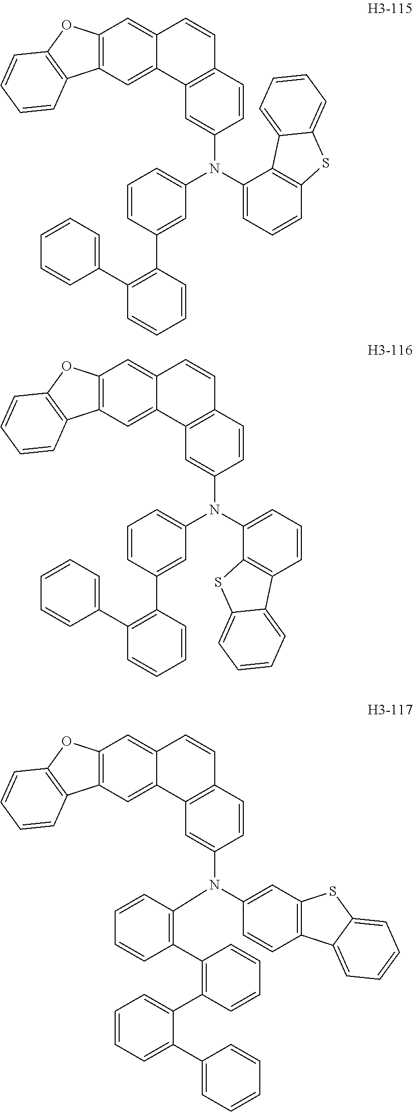

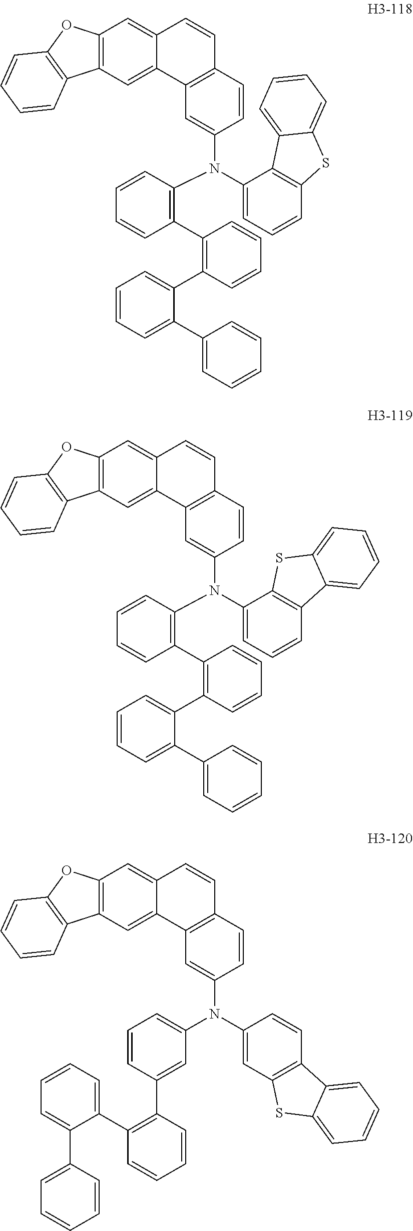 [SMILES: c1ccc(-c2ccccc2-c2ccccc2-c2ccccc2N(c2ccc3ccc4cc5oc6ccccc6c5cc4c3c2)c2cccc3sc4ccccc4c23)cc1 ; c1ccc(-c2ccccc2-c2ccccc2-c2ccccc2N(c2ccc3ccc4cc5oc6ccccc6c5cc4c3c2)c2cccc3c2sc2ccccc23)cc1 ; c1ccc(-c2ccccc2-c2ccccc2-c2cccc(N(c3ccc4c(c3)sc3ccccc34)c3ccc4ccc5cc6oc7ccccc7c6cc5c4c3)c2)cc1]

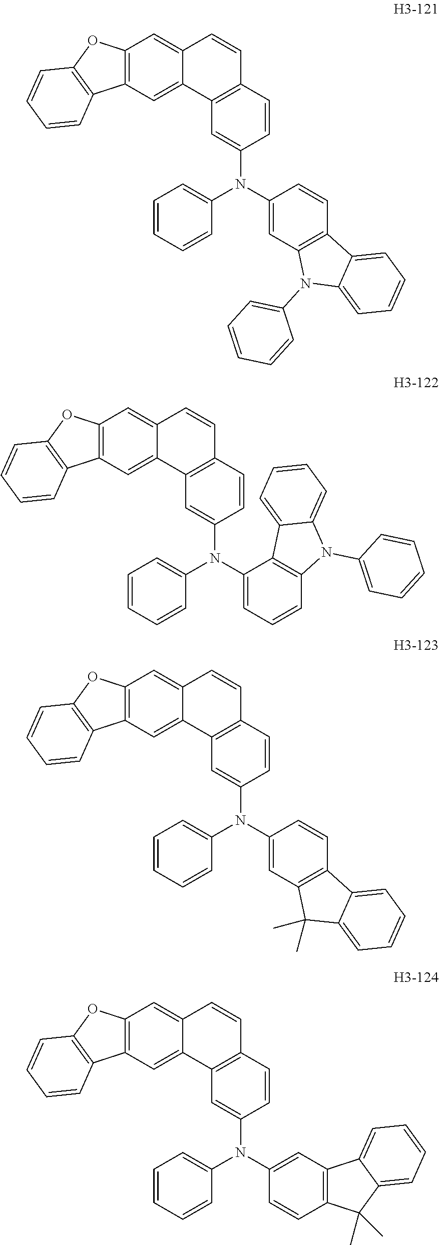

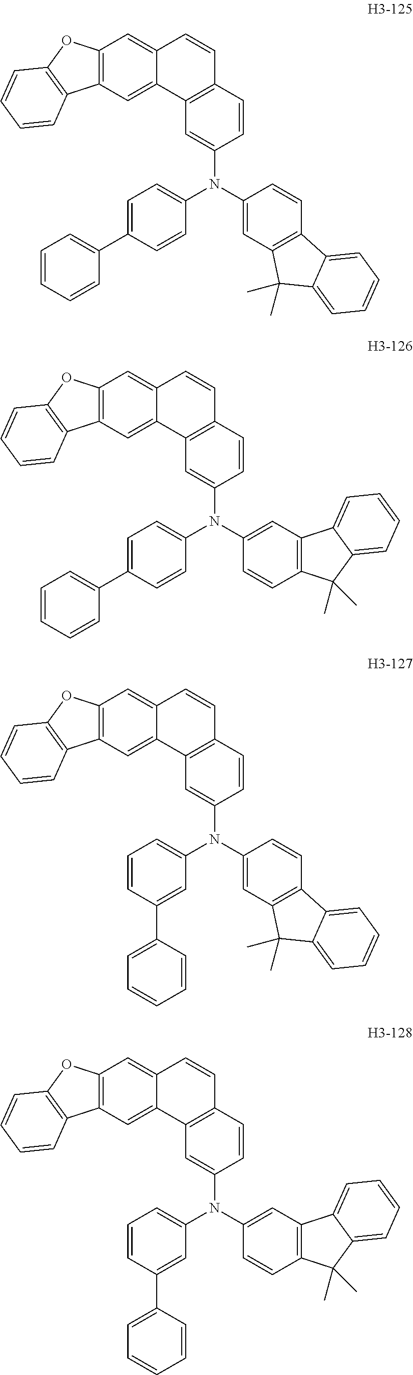 [SMILES: CC1(C)c2ccccc2-c2ccc(N(c3ccc(-c4ccccc4)cc3)c3ccc4ccc5cc6oc7ccccc7c6cc5c4c3)cc21 ; CC1(C)c2ccccc2-c2cc(N(c3ccc(-c4ccccc4)cc3)c3ccc4ccc5cc6oc7ccccc7c6cc5c4c3)ccc21 ; CC1(C)c2ccccc2-c2ccc(N(c3cccc(-c4ccccc4)c3)c3ccc4ccc5cc6oc7ccccc7c6cc5c4c3)cc21 ; CC1(C)c2ccccc2-c2cc(N(c3cccc(-c4ccccc4)c3)c3ccc4ccc5cc6oc7ccccc7c6cc5c4c3)ccc21]

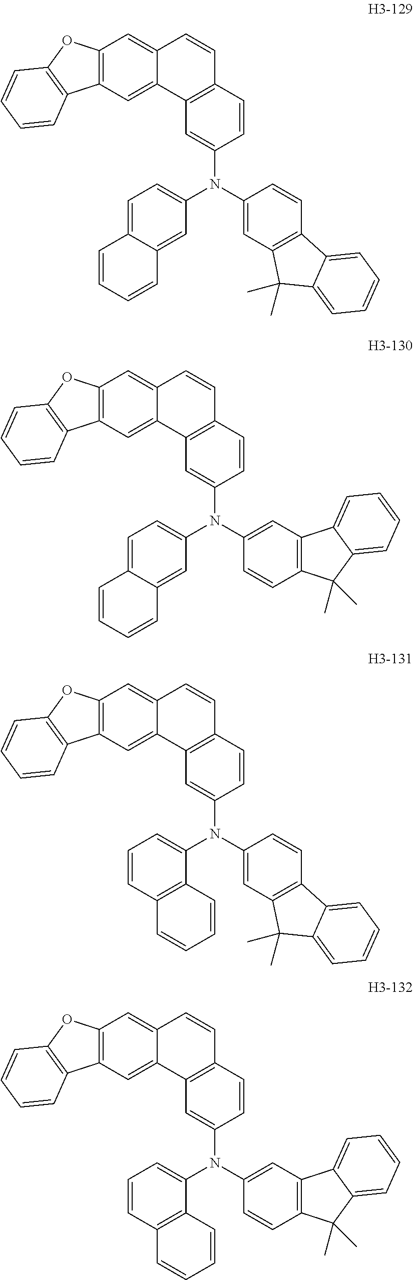

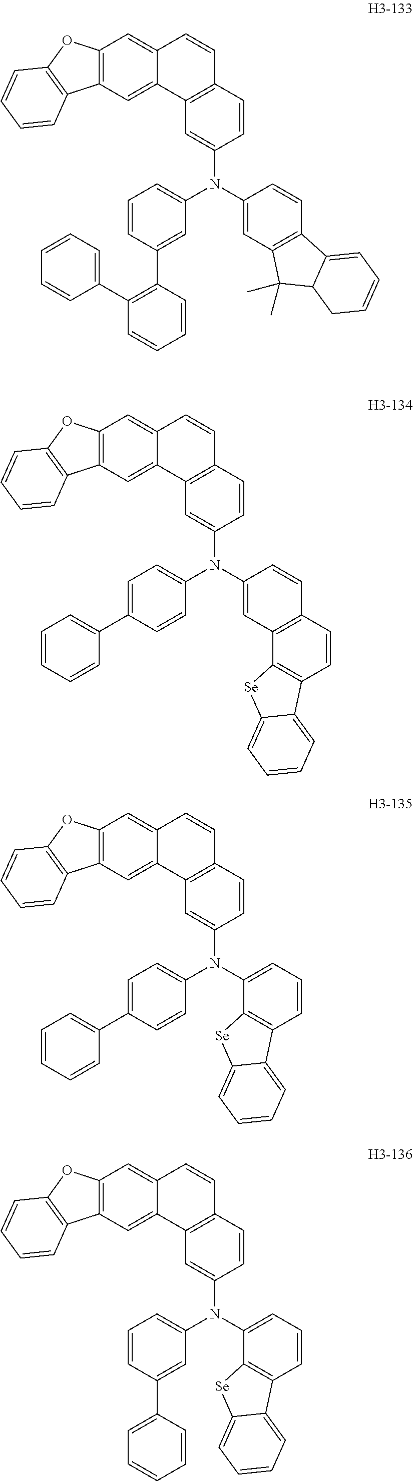 [SMILES: CC1(C)c2cc(N(c3cccc(-c4ccccc4-c4ccccc4)c3)c3ccc4ccc5cc6oc7ccccc7c6cc5c4c3)ccc2C2=CC=CCC21 ; c1ccc(-c2ccc(N(c3ccc4ccc5cc6oc7ccccc7c6cc5c4c3)c3ccc4ccc5c6ccccc6[se]c5c4c3)cc2)cc1 ; c1ccc(-c2ccc(N(c3ccc4ccc5cc6oc7ccccc7c6cc5c4c3)c3cccc4c3[se]c3ccccc34)cc2)cc1 ; c1ccc(-c2cccc(N(c3ccc4ccc5cc6oc7ccccc7c6cc5c4c3)c3cccc4c3[se]c3ccccc34)c2)cc1]

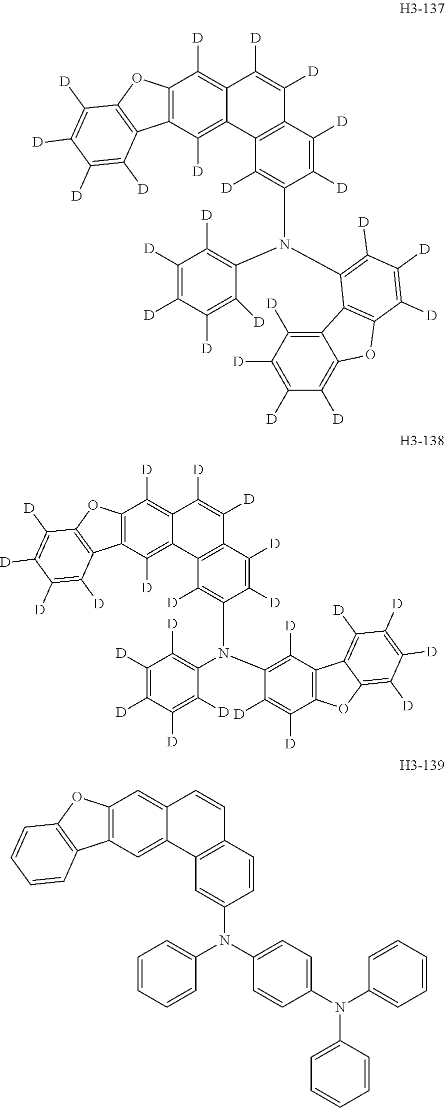 [SMILES: [2H]c1c([2H])c([2H])c(N(c2c([2H])c([2H])c3c([2H])c([2H])c4c([2H])c5oc6c([2H])c([2H])c([2H])c([2H])c6c5c([2H])c4c3c2[2H])c2c([2H])c([2H])c([2H])c3oc4c([2H])c([2H])c([2H])c([2H])c4c23)c([2H])c1[2H] ; [2H]c1c([2H])c([2H])c(N(c2c([2H])c([2H])c3oc4c([2H])c([2H])c([2H])c([2H])c4c3c2[2H])c2c([2H])c([2H])c3c([2H])c([2H])c4c([2H])c5oc6c([2H])c([2H])c([2H])c([2H])c6c5c([2H])c4c3c2[2H])c([2H])c1[2H] ; c1ccc(N(c2ccccc2)c2ccc(N(c3ccccc3)c3ccc4ccc5cc6oc7ccccc7c6cc5c4c3)cc2)cc1]

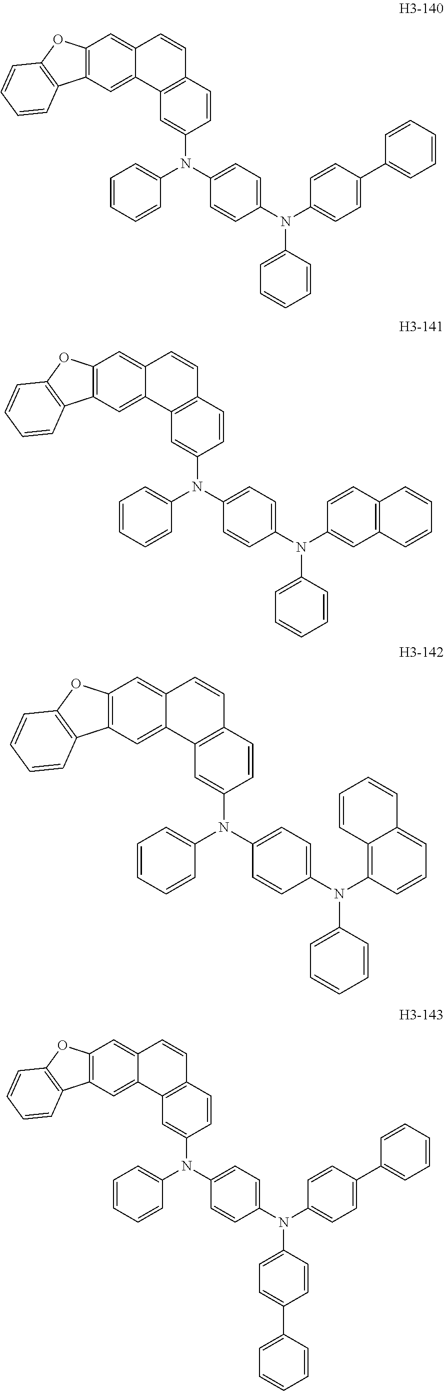 [SMILES: c1ccc(-c2ccc(N(c3ccccc3)c3ccc(N(c4ccccc4)c4ccc5ccc6cc7oc8ccccc8c7cc6c5c4)cc3)cc2)cc1 ; c1ccc(N(c2ccc(N(c3ccccc3)c3ccc4ccc5cc6oc7ccccc7c6cc5c4c3)cc2)c2ccc3ccccc3c2)cc1 ; c1ccc(N(c2ccc(N(c3ccccc3)c3cccc4ccccc34)cc2)c2ccc3ccc4cc5oc6ccccc6c5cc4c3c2)cc1 ; c1ccc(-c2ccc(N(c3ccc(-c4ccccc4)cc3)c3ccc(N(c4ccccc4)c4ccc5ccc6cc7oc8ccccc8c7cc6c5c4)cc3)cc2)cc1]

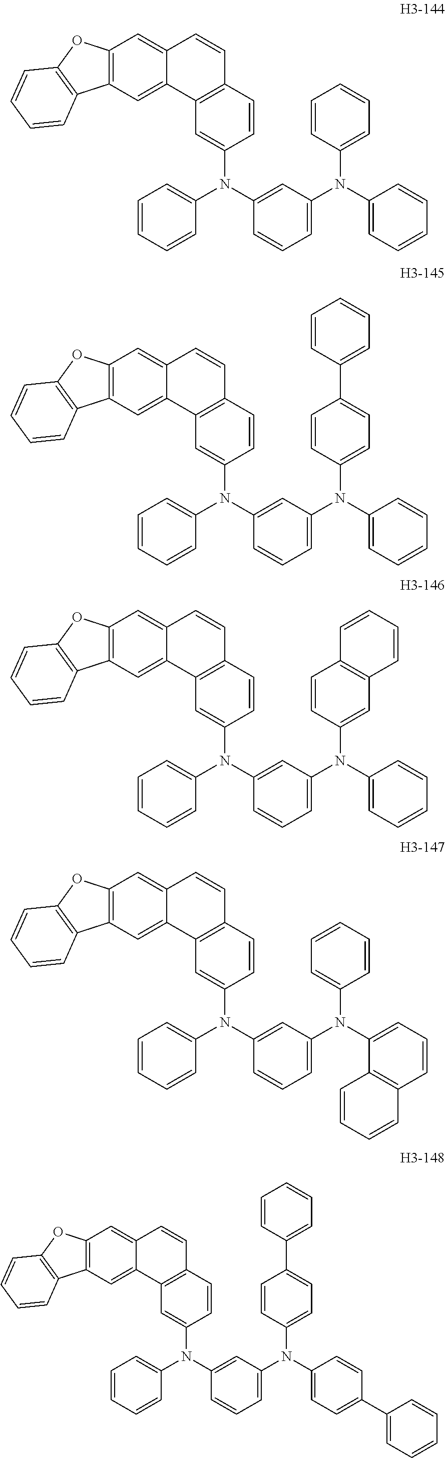 [SMILES: c1ccc(N(c2ccccc2)c2cccc(N(c3ccccc3)c3ccc4ccc5cc6oc7ccccc7c6cc5c4c3)c2)cc1 ; c1ccc(-c2ccc(N(c3ccccc3)c3cccc(N(c4ccccc4)c4ccc5ccc6cc7oc8ccccc8c7cc6c5c4)c3)cc2)cc1 ; c1ccc(N(c2cccc(N(c3ccccc3)c3ccc4ccc5cc6oc7ccccc7c6cc5c4c3)c2)c2ccc3ccccc3c2)cc1 ; c1ccc(N(c2cccc(N(c3ccccc3)c3cccc4ccccc34)c2)c2ccc3ccc4cc5oc6ccccc6c5cc4c3c2)cc1 ; c1ccc(-c2ccc(N(c3ccc(-c4ccccc4)cc3)c3cccc(N(c4ccccc4)c4ccc5ccc6cc7oc8ccccc8c7cc6c5c4)c3)cc2)cc1]

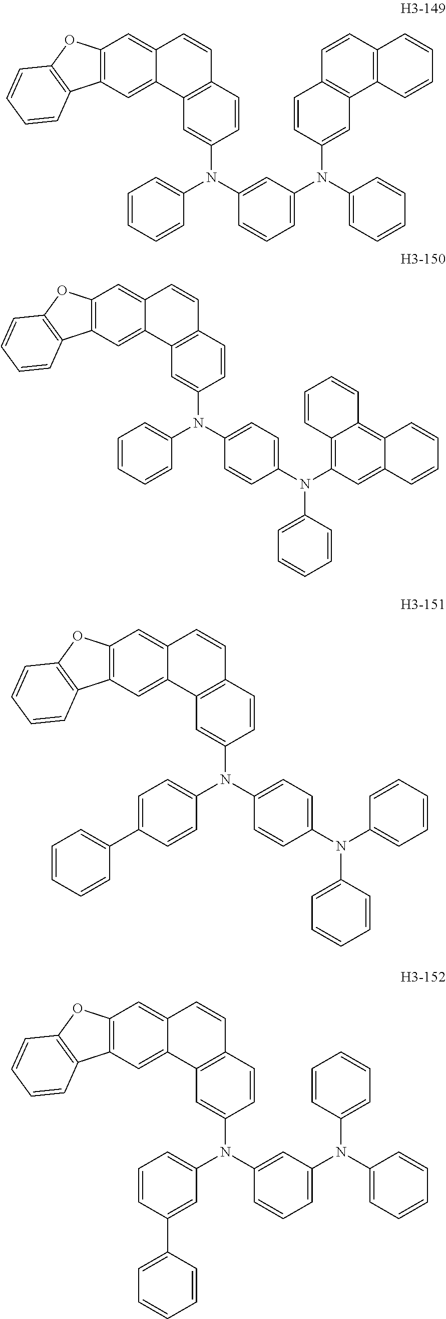 [SMILES: c1ccc(N(c2cccc(N(c3ccccc3)c3ccc4ccc5cc6oc7ccccc7c6cc5c4c3)c2)c2ccc3ccc4ccccc4c3c2)cc1 ; c1ccc(N(c2ccc(N(c3ccccc3)c3cc4ccccc4c4ccccc34)cc2)c2ccc3ccc4cc5oc6ccccc6c5cc4c3c2)cc1 ; c1ccc(-c2ccc(N(c3ccc(N(c4ccccc4)c4ccccc4)cc3)c3ccc4ccc5cc6oc7ccccc7c6cc5c4c3)cc2)cc1 ; c1ccc(-c2cccc(N(c3cccc(N(c4ccccc4)c4ccccc4)c3)c3ccc4ccc5cc6oc7ccccc7c6cc5c4c3)c2)cc1]

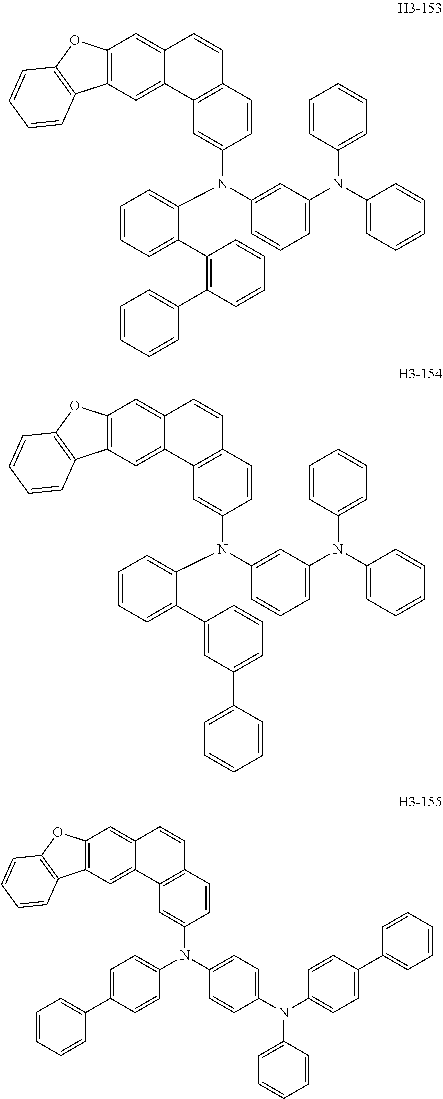

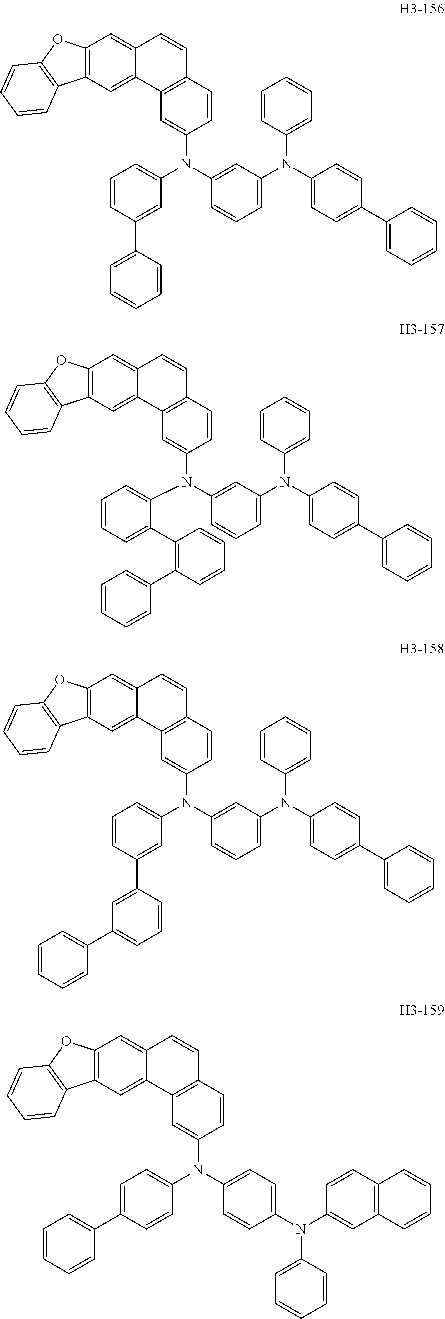 [SMILES: c1ccc(-c2ccc(N(c3ccccc3)c3cccc(N(c4cccc(-c5ccccc5)c4)c4ccc5ccc6cc7oc8ccccc8c7cc6c5c4)c3)cc2)cc1 ; c1ccc(-c2ccc(N(c3ccccc3)c3cccc(N(c4ccc5ccc6cc7oc8ccccc8c7cc6c5c4)c4ccccc4-c4ccccc4-c4ccccc4)c3)cc2)cc1 ; c1ccc(-c2ccc(N(c3ccccc3)c3cccc(N(c4cccc(-c5cccc(-c6ccccc6)c5)c4)c4ccc5ccc6cc7oc8ccccc8c7cc6c5c4)c3)cc2)cc1 ; c1ccc(-c2ccc(N(c3ccc(N(c4ccccc4)c4ccc5ccccc5c4)cc3)c3ccc4ccc5cc6oc7ccccc7c6cc5c4c3)cc2)cc1]

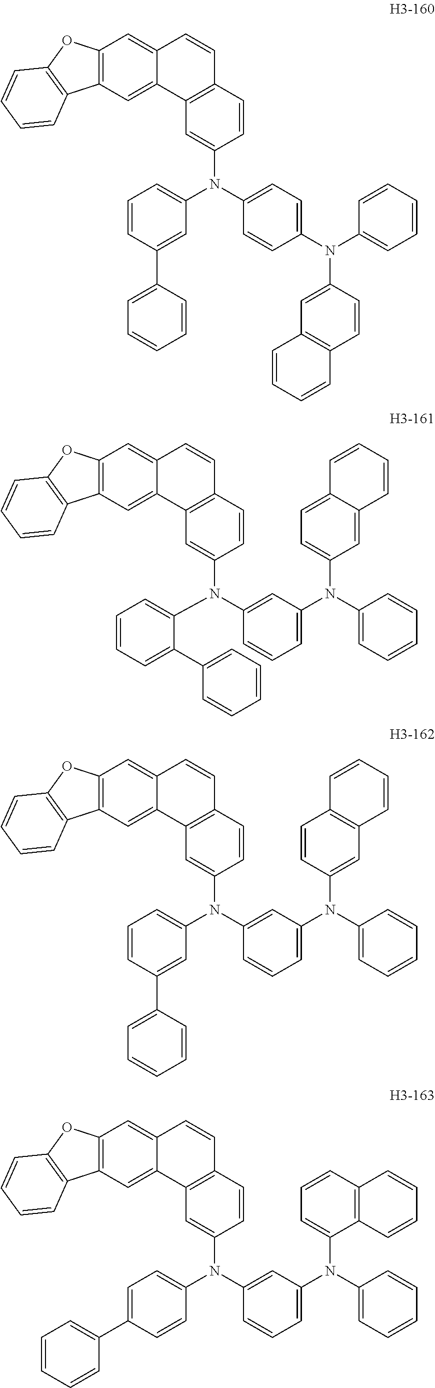 [SMILES: c1ccc(-c2cccc(N(c3ccc(N(c4ccccc4)c4ccc5ccccc5c4)cc3)c3ccc4ccc5cc6oc7ccccc7c6cc5c4c3)c2)cc1 ; c1ccc(-c2ccccc2N(c2cccc(N(c3ccccc3)c3ccc4ccccc4c3)c2)c2ccc3ccc4cc5oc6ccccc6c5cc4c3c2)cc1 ; c1ccc(-c2cccc(N(c3cccc(N(c4ccccc4)c4ccc5ccccc5c4)c3)c3ccc4ccc5cc6oc7ccccc7c6cc5c4c3)c2)cc1 ; c1ccc(-c2ccc(N(c3cccc(N(c4ccccc4)c4cccc5ccccc45)c3)c3ccc4ccc5cc6oc7ccccc7c6cc5c4c3)cc2)cc1]

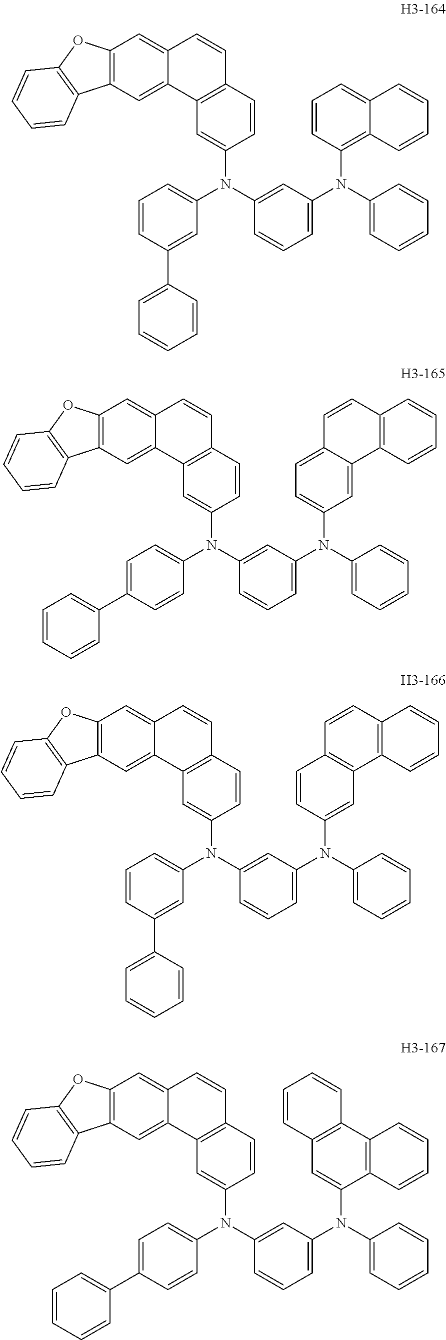 [SMILES: c1ccc(-c2cccc(N(c3cccc(N(c4ccccc4)c4cccc5ccccc45)c3)c3ccc4ccc5cc6oc7ccccc7c6cc5c4c3)c2)cc1 ; c1ccc(-c2ccc(N(c3cccc(N(c4ccccc4)c4ccc5ccc6ccccc6c5c4)c3)c3ccc4ccc5cc6oc7ccccc7c6cc5c4c3)cc2)cc1 ; c1ccc(-c2cccc(N(c3cccc(N(c4ccccc4)c4ccc5ccc6ccccc6c5c4)c3)c3ccc4ccc5cc6oc7ccccc7c6cc5c4c3)c2)cc1 ; c1ccc(-c2ccc(N(c3cccc(N(c4ccccc4)c4cc5ccccc5c5ccccc45)c3)c3ccc4ccc5cc6oc7ccccc7c6cc5c4c3)cc2)cc1]

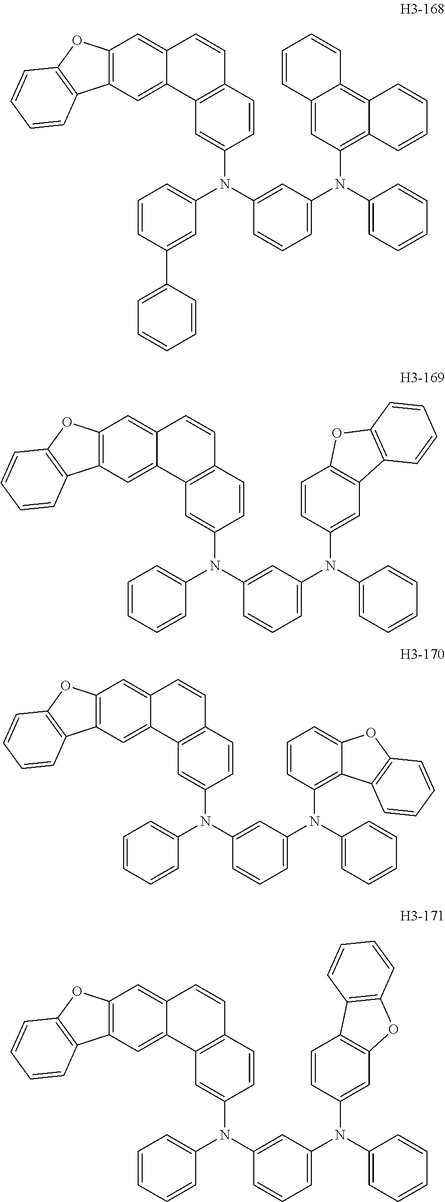 [SMILES: c1ccc(-c2cccc(N(c3cccc(N(c4ccccc4)c4cc5ccccc5c5ccccc45)c3)c3ccc4ccc5cc6oc7ccccc7c6cc5c4c3)c2)cc1 ; c1ccc(N(c2cccc(N(c3ccccc3)c3ccc4oc5ccccc5c4c3)c2)c2ccc3ccc4cc5oc6ccccc6c5cc4c3c2)cc1 ; c1ccc(N(c2cccc(N(c3ccccc3)c3cccc4oc5ccccc5c34)c2)c2ccc3ccc4cc5oc6ccccc6c5cc4c3c2)cc1 ; c1ccc(N(c2cccc(N(c3ccccc3)c3ccc4ccc5cc6oc7ccccc7c6cc5c4c3)c2)c2ccc3c(c2)oc2ccccc23)cc1]

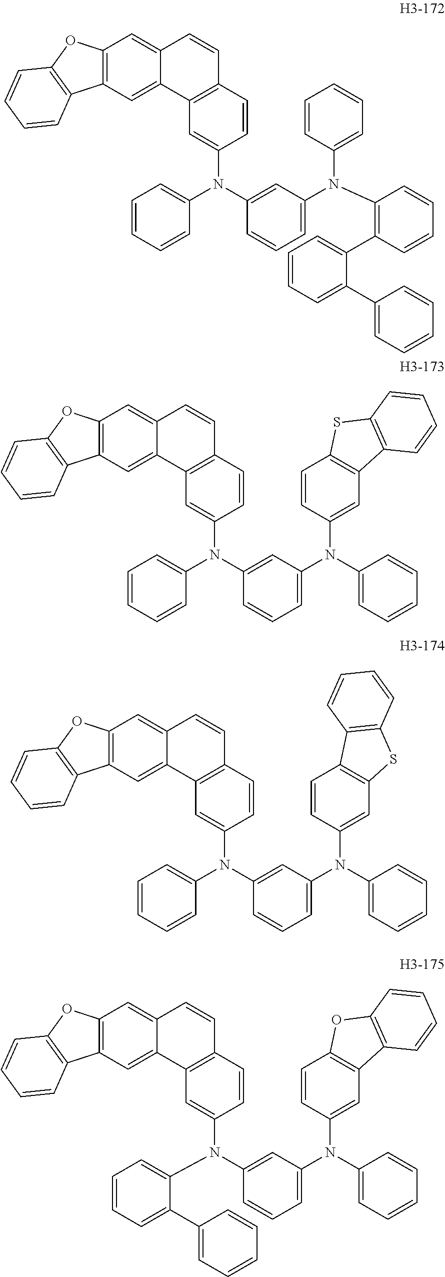

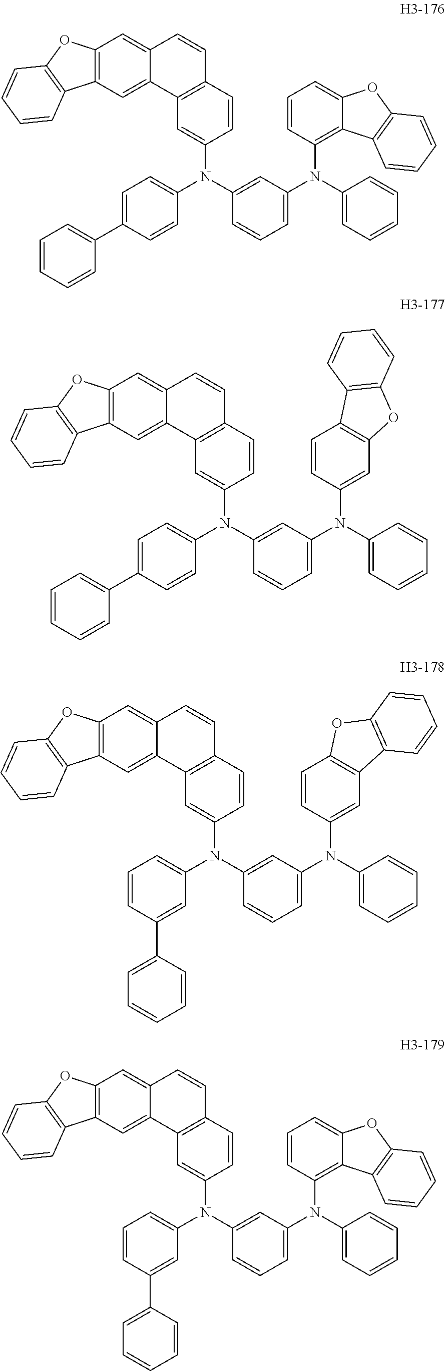 [SMILES: c1ccc(-c2ccc(N(c3cccc(N(c4ccccc4)c4cccc5oc6ccccc6c45)c3)c3ccc4ccc5cc6oc7ccccc7c6cc5c4c3)cc2)cc1 ; c1ccc(-c2ccc(N(c3cccc(N(c4ccccc4)c4ccc5c(c4)oc4ccccc45)c3)c3ccc4ccc5cc6oc7ccccc7c6cc5c4c3)cc2)cc1 ; c1ccc(-c2cccc(N(c3cccc(N(c4ccccc4)c4ccc5oc6ccccc6c5c4)c3)c3ccc4ccc5cc6oc7ccccc7c6cc5c4c3)c2)cc1 ; c1ccc(-c2cccc(N(c3cccc(N(c4ccccc4)c4cccc5oc6ccccc6c45)c3)c3ccc4ccc5cc6oc7ccccc7c6cc5c4c3)c2)cc1]

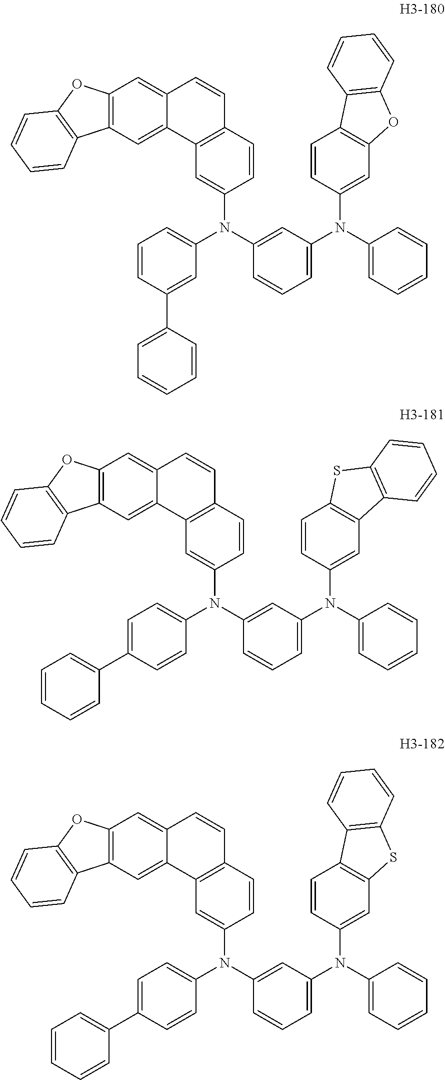 [SMILES: c1ccc(-c2cccc(N(c3cccc(N(c4ccccc4)c4ccc5c(c4)oc4ccccc45)c3)c3ccc4ccc5cc6oc7ccccc7c6cc5c4c3)c2)cc1 ; c1ccc(-c2ccc(N(c3cccc(N(c4ccccc4)c4ccc5sc6ccccc6c5c4)c3)c3ccc4ccc5cc6oc7ccccc7c6cc5c4c3)cc2)cc1 ; c1ccc(-c2ccc(N(c3cccc(N(c4ccccc4)c4ccc5c(c4)sc4ccccc45)c3)c3ccc4ccc5cc6oc7ccccc7c6cc5c4c3)cc2)cc1]

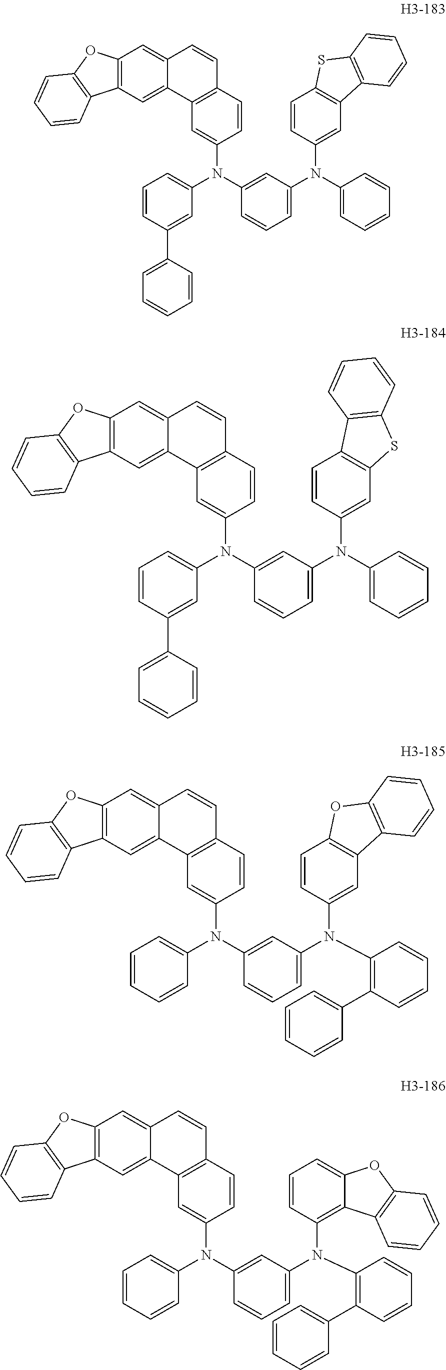 [SMILES: c1ccc(-c2cccc(N(c3cccc(N(c4ccccc4)c4ccc5sc6ccccc6c5c4)c3)c3ccc4ccc5cc6oc7ccccc7c6cc5c4c3)c2)cc1 ; c1ccc(-c2cccc(N(c3cccc(N(c4ccccc4)c4ccc5c(c4)sc4ccccc45)c3)c3ccc4ccc5cc6oc7ccccc7c6cc5c4c3)c2)cc1 ; c1ccc(-c2ccccc2N(c2cccc(N(c3ccccc3)c3ccc4ccc5cc6oc7ccccc7c6cc5c4c3)c2)c2ccc3oc4ccccc4c3c2)cc1 ; c1ccc(-c2ccccc2N(c2cccc(N(c3ccccc3)c3ccc4ccc5cc6oc7ccccc7c6cc5c4c3)c2)c2cccc3oc4ccccc4c23)cc1]

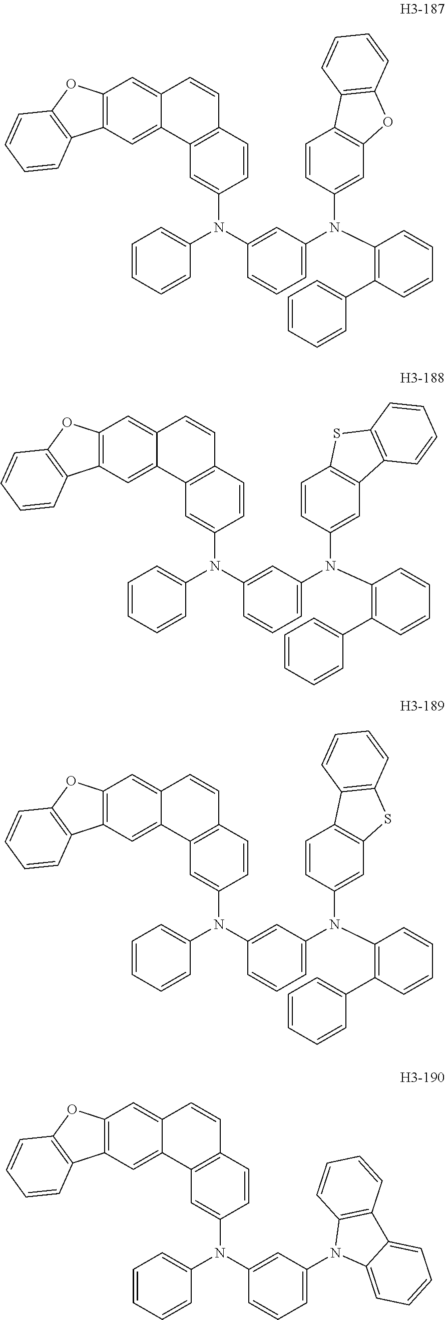 [SMILES: c1ccc(-c2ccccc2N(c2cccc(N(c3ccccc3)c3ccc4ccc5cc6oc7ccccc7c6cc5c4c3)c2)c2ccc3c(c2)oc2ccccc23)cc1 ; c1ccc(-c2ccccc2N(c2cccc(N(c3ccccc3)c3ccc4ccc5cc6oc7ccccc7c6cc5c4c3)c2)c2ccc3sc4ccccc4c3c2)cc1 ; c1ccc(-c2ccccc2N(c2cccc(N(c3ccccc3)c3ccc4ccc5cc6oc7ccccc7c6cc5c4c3)c2)c2ccc3c(c2)sc2ccccc23)cc1 ; c1ccc(N(c2cccc(-n3c4ccccc4c4ccccc43)c2)c2ccc3ccc4cc5oc6ccccc6c5cc4c3c2)cc1]

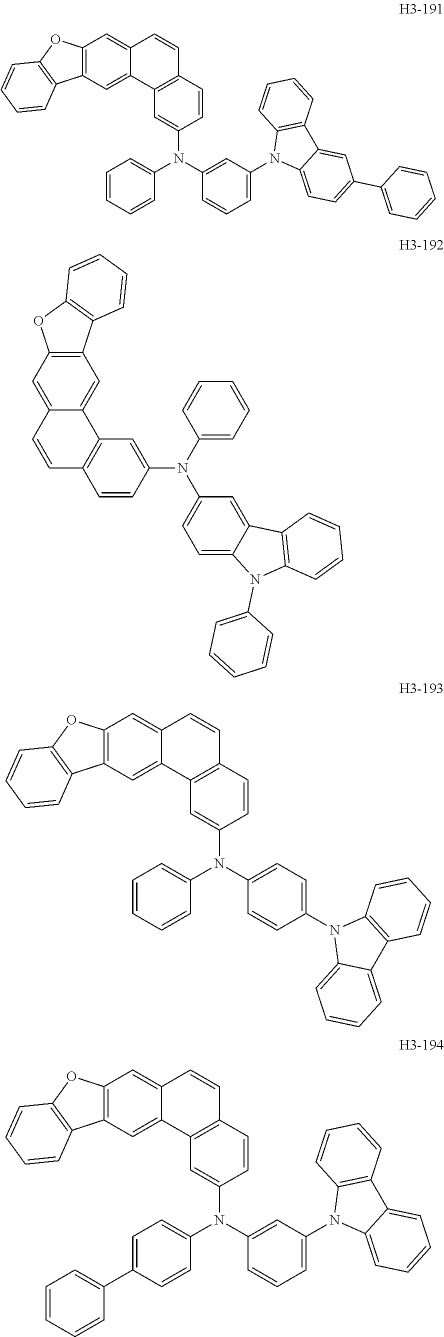 [SMILES: c1ccc(-c2ccc3c(c2)c2ccccc2n3-c2cccc(N(c3ccccc3)c3ccc4ccc5cc6oc7ccccc7c6cc5c4c3)c2)cc1 ; c1ccc(N(c2ccc3ccc4cc5oc6ccccc6c5cc4c3c2)c2ccc3c(c2)c2ccccc2n3-c2ccccc2)cc1 ; c1ccc(N(c2ccc(-n3c4ccccc4c4ccccc43)cc2)c2ccc3ccc4cc5oc6ccccc6c5cc4c3c2)cc1 ; c1ccc(-c2ccc(N(c3cccc(-n4c5ccccc5c5ccccc54)c3)c3ccc4ccc5cc6oc7ccccc7c6cc5c4c3)cc2)cc1]

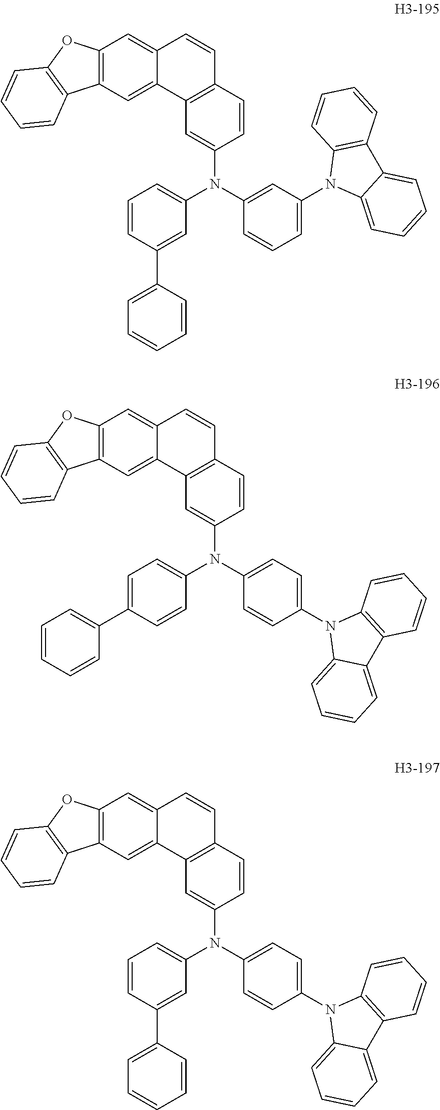 [SMILES: c1ccc(-c2cccc(N(c3cccc(-n4c5ccccc5c5ccccc54)c3)c3ccc4ccc5cc6oc7ccccc7c6cc5c4c3)c2)cc1 ; c1ccc(-c2ccc(N(c3ccc(-n4c5ccccc5c5ccccc54)cc3)c3ccc4ccc5cc6oc7ccccc7c6cc5c4c3)cc2)cc1 ; c1ccc(-c2cccc(N(c3ccc(-n4c5ccccc5c5ccccc54)cc3)c3ccc4ccc5cc6oc7ccccc7c6cc5c4c3)c2)cc1]

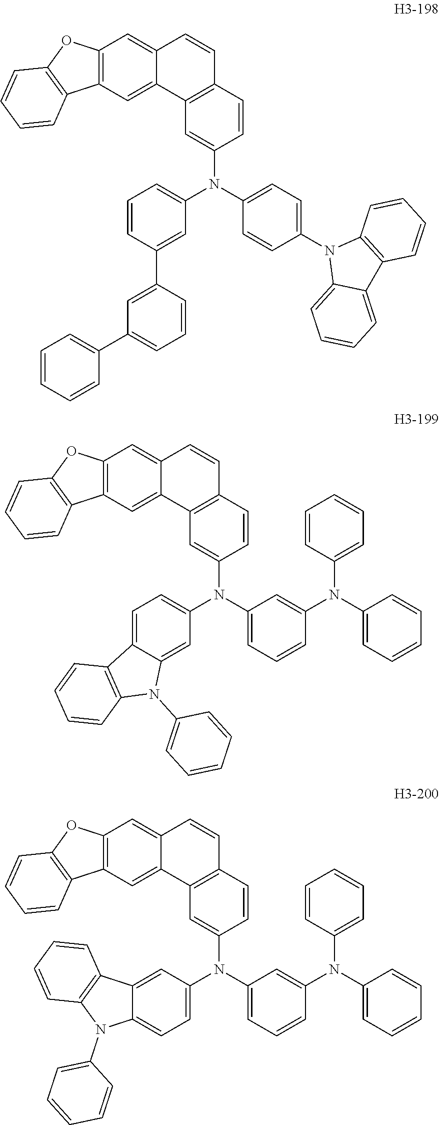

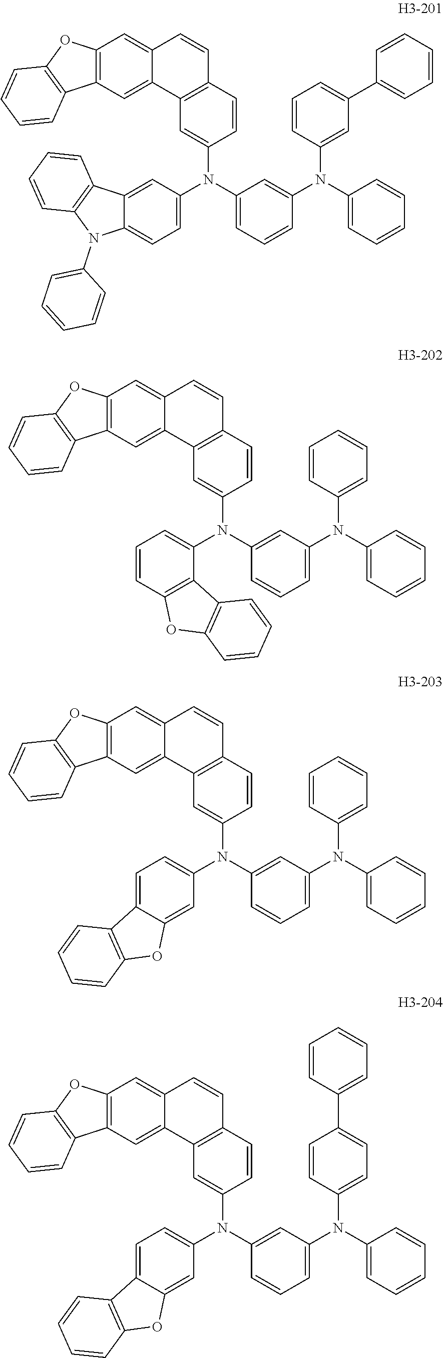 [SMILES: c1ccc(-c2cccc(N(c3ccccc3)c3cccc(N(c4ccc5ccc6cc7oc8ccccc8c7cc6c5c4)c4ccc5c(c4)c4ccccc4n5-c4ccccc4)c3)c2)cc1 ; c1ccc(N(c2ccccc2)c2cccc(N(c3ccc4ccc5cc6oc7ccccc7c6cc5c4c3)c3cccc4oc5ccccc5c34)c2)cc1 ; c1ccc(N(c2ccccc2)c2cccc(N(c3ccc4c(c3)oc3ccccc34)c3ccc4ccc5cc6oc7ccccc7c6cc5c4c3)c2)cc1 ; c1ccc(-c2ccc(N(c3ccccc3)c3cccc(N(c4ccc5c(c4)oc4ccccc45)c4ccc5ccc6cc7oc8ccccc8c7cc6c5c4)c3)cc2)cc1]

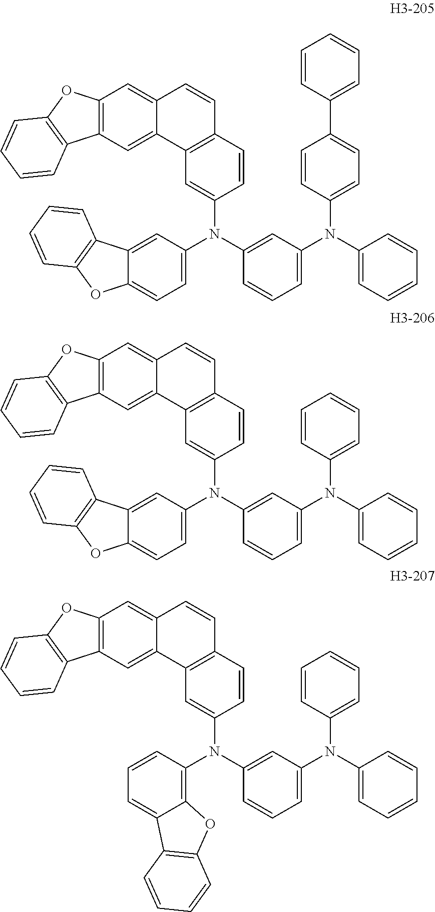 [SMILES: c1ccc(-c2ccc(N(c3ccccc3)c3cccc(N(c4ccc5ccc6cc7oc8ccccc8c7cc6c5c4)c4ccc5oc6ccccc6c5c4)c3)cc2)cc1 ; c1ccc(N(c2ccccc2)c2cccc(N(c3ccc4ccc5cc6oc7ccccc7c6cc5c4c3)c3ccc4oc5ccccc5c4c3)c2)cc1 ; c1ccc(N(c2ccccc2)c2cccc(N(c3ccc4ccc5cc6oc7ccccc7c6cc5c4c3)c3cccc4c3oc3ccccc34)c2)cc1]

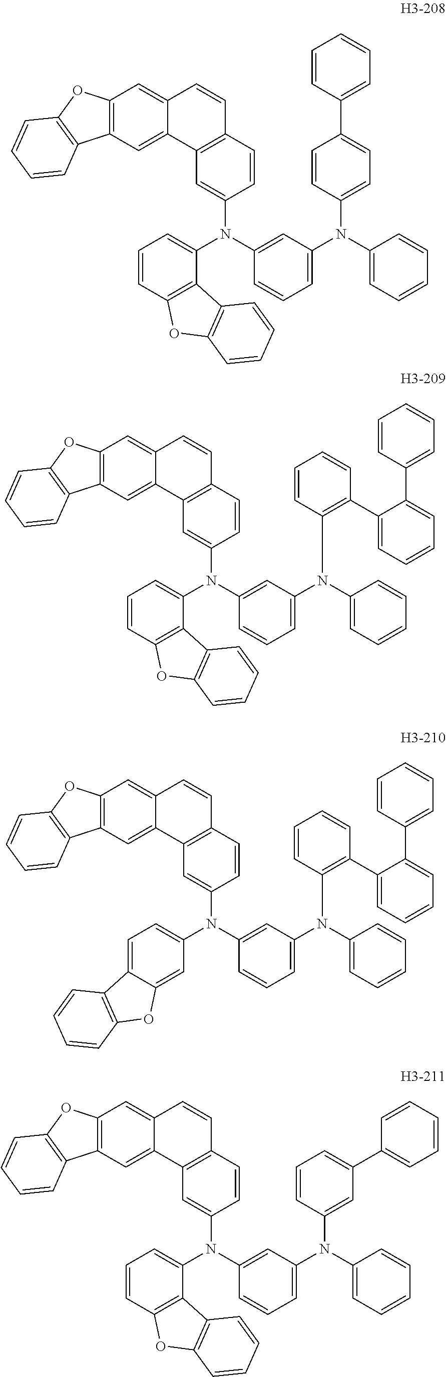 [SMILES: c1ccc(-c2ccc(N(c3ccccc3)c3cccc(N(c4ccc5ccc6cc7oc8ccccc8c7cc6c5c4)c4cccc5oc6ccccc6c45)c3)cc2)cc1 ; c1ccc(-c2ccccc2-c2ccccc2N(c2ccccc2)c2cccc(N(c3ccc4ccc5cc6oc7ccccc7c6cc5c4c3)c3cccc4oc5ccccc5c34)c2)cc1 ; c1ccc(-c2ccccc2-c2ccccc2N(c2ccccc2)c2cccc(N(c3ccc4c(c3)oc3ccccc34)c3ccc4ccc5cc6oc7ccccc7c6cc5c4c3)c2)cc1 ; c1ccc(-c2cccc(N(c3ccccc3)c3cccc(N(c4ccc5ccc6cc7oc8ccccc8c7cc6c5c4)c4cccc5oc6ccccc6c45)c3)c2)cc1]

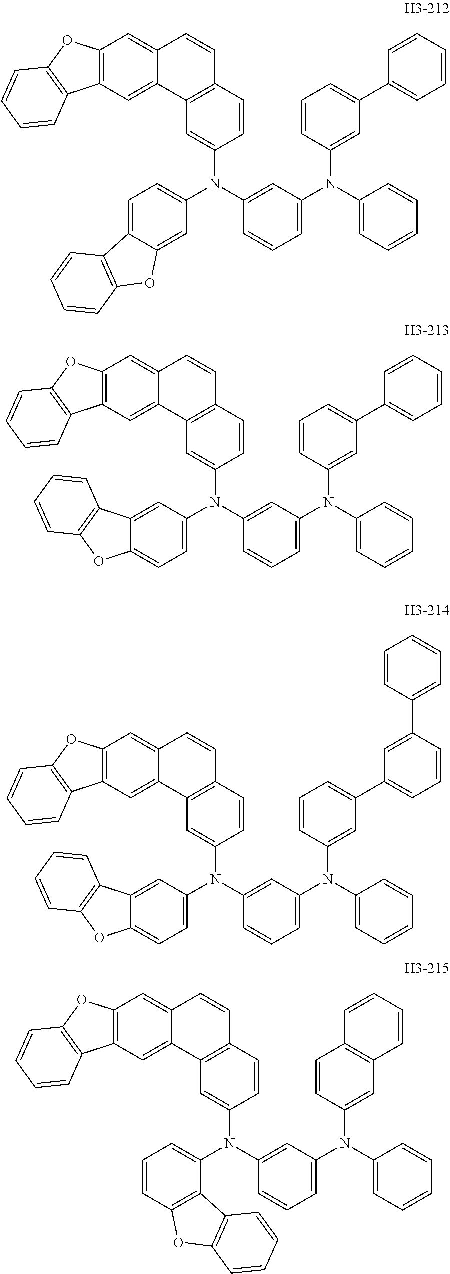 [SMILES: c1ccc(-c2cccc(N(c3ccccc3)c3cccc(N(c4ccc5c(c4)oc4ccccc45)c4ccc5ccc6cc7oc8ccccc8c7cc6c5c4)c3)c2)cc1 ; c1ccc(-c2cccc(N(c3ccccc3)c3cccc(N(c4ccc5ccc6cc7oc8ccccc8c7cc6c5c4)c4ccc5oc6ccccc6c5c4)c3)c2)cc1 ; c1ccc(-c2cccc(-c3cccc(N(c4ccccc4)c4cccc(N(c5ccc6ccc7cc8oc9ccccc9c8cc7c6c5)c5ccc6oc7ccccc7c6c5)c4)c3)c2)cc1 ; c1ccc(N(c2cccc(N(c3ccc4ccc5cc6oc7ccccc7c6cc5c4c3)c3cccc4oc5ccccc5c34)c2)c2ccc3ccccc3c2)cc1]

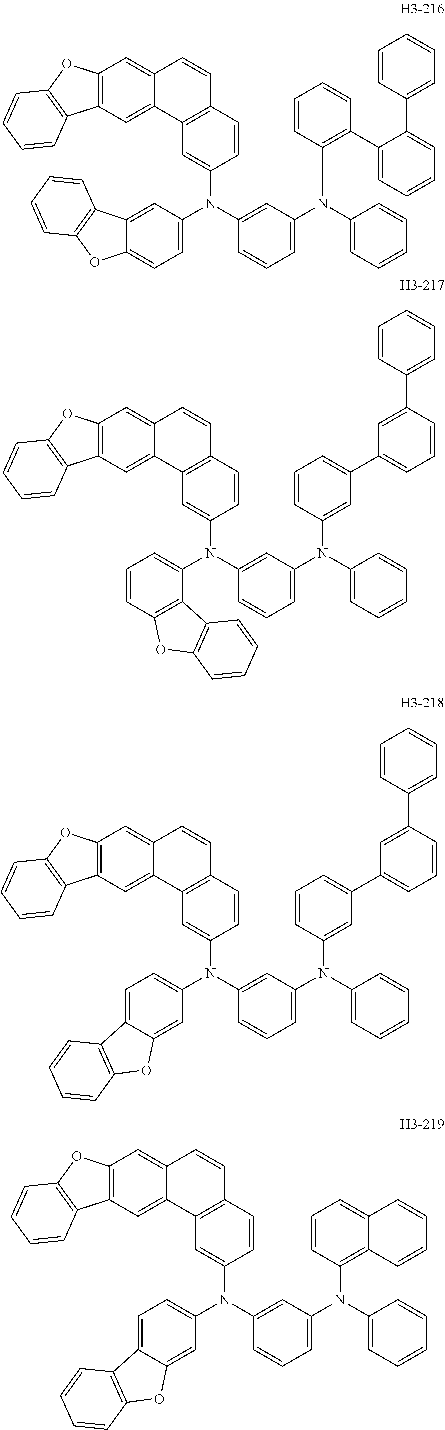 [SMILES: c1ccc(-c2ccccc2-c2ccccc2N(c2ccccc2)c2cccc(N(c3ccc4ccc5cc6oc7ccccc7c6cc5c4c3)c3ccc4oc5ccccc5c4c3)c2)cc1 ; c1ccc(-c2cccc(-c3cccc(N(c4ccccc4)c4cccc(N(c5ccc6ccc7cc8oc9ccccc9c8cc7c6c5)c5cccc6oc7ccccc7c56)c4)c3)c2)cc1 ; c1ccc(-c2cccc(-c3cccc(N(c4ccccc4)c4cccc(N(c5ccc6c(c5)oc5ccccc56)c5ccc6ccc7cc8oc9ccccc9c8cc7c6c5)c4)c3)c2)cc1 ; c1ccc(N(c2cccc(N(c3ccc4c(c3)oc3ccccc34)c3ccc4ccc5cc6oc7ccccc7c6cc5c4c3)c2)c2cccc3ccccc23)cc1]

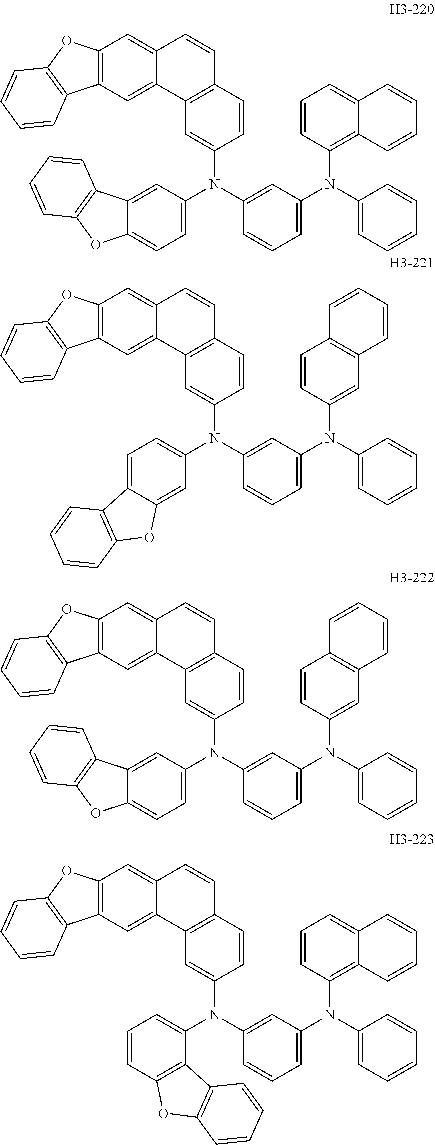

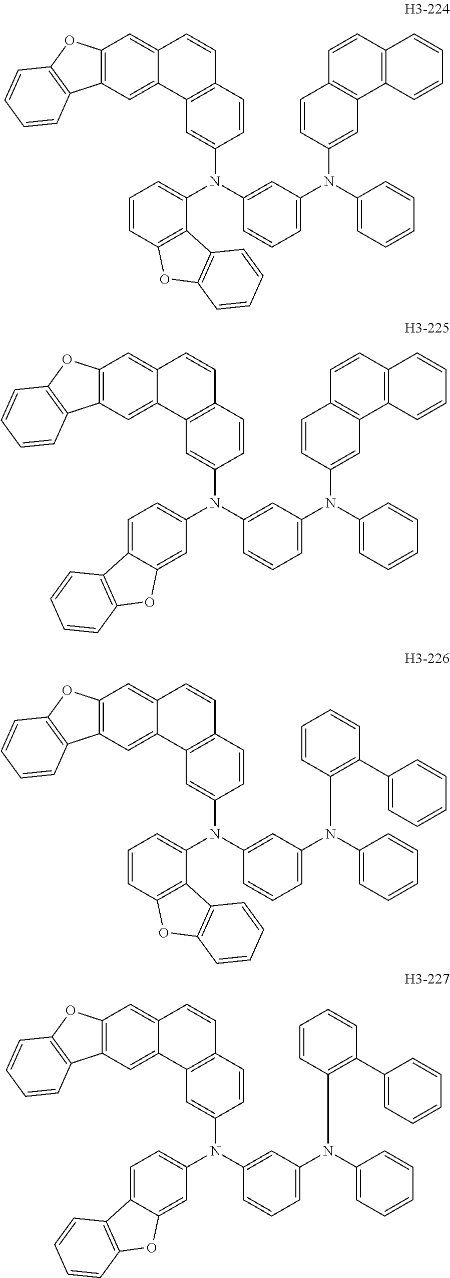 [SMILES: c1ccc(N(c2cccc(N(c3ccc4ccc5cc6oc7ccccc7c6cc5c4c3)c3cccc4oc5ccccc5c34)c2)c2ccc3ccc4ccccc4c3c2)cc1 ; c1ccc(N(c2cccc(N(c3ccc4c(c3)oc3ccccc34)c3ccc4ccc5cc6oc7ccccc7c6cc5c4c3)c2)c2ccc3ccc4ccccc4c3c2)cc1 ; c1ccc(-c2ccccc2N(c2ccccc2)c2cccc(N(c3ccc4ccc5cc6oc7ccccc7c6cc5c4c3)c3cccc4oc5ccccc5c34)c2)cc1 ; c1ccc(-c2ccccc2N(c2ccccc2)c2cccc(N(c3ccc4c(c3)oc3ccccc34)c3ccc4ccc5cc6oc7ccccc7c6cc5c4c3)c2)cc1]

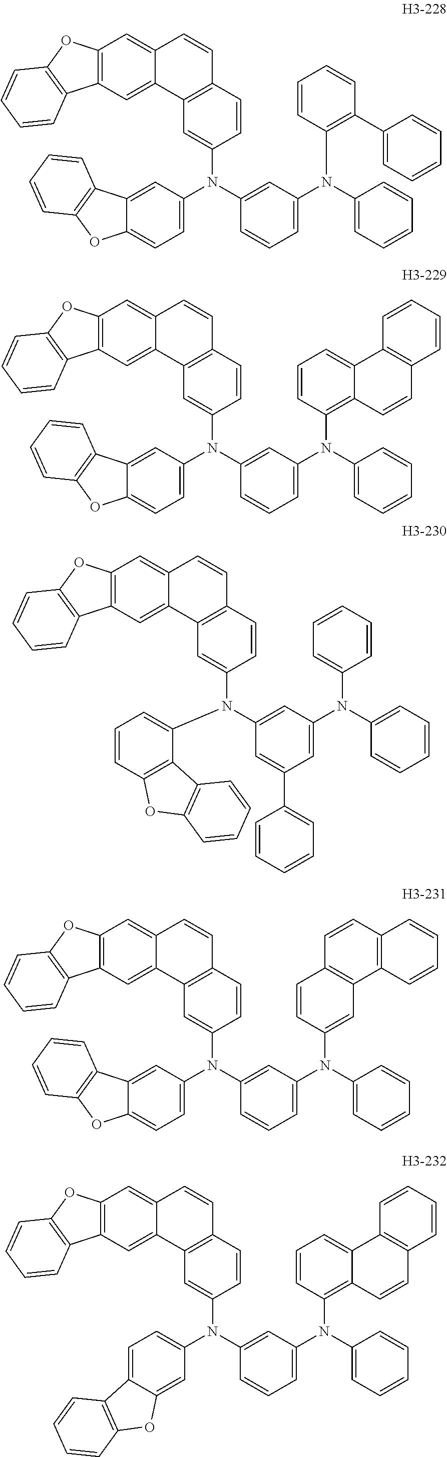

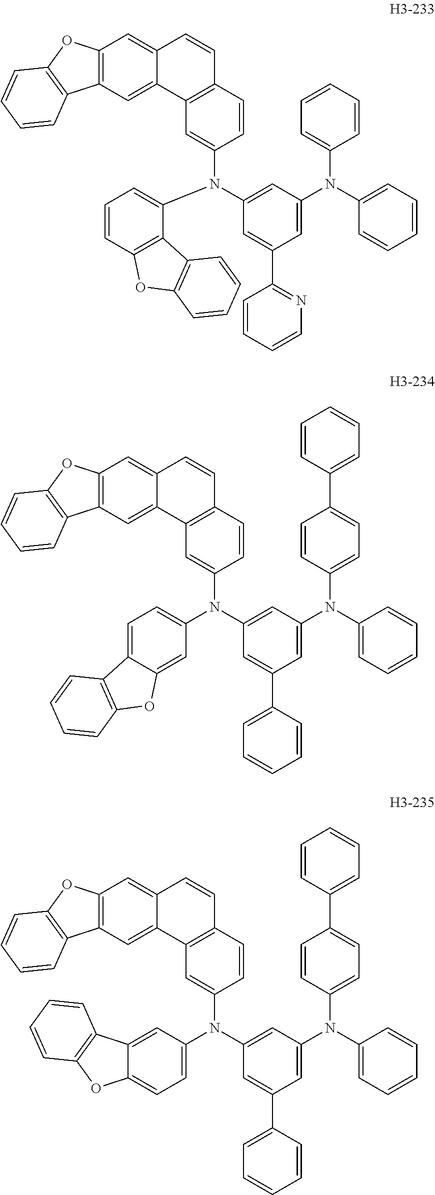 [SMILES: c1ccc(N(c2ccccc2)c2cc(-c3ccccn3)cc(N(c3ccc4ccc5cc6oc7ccccc7c6cc5c4c3)c3cccc4oc5ccccc5c34)c2)cc1 ; c1ccc(-c2ccc(N(c3ccccc3)c3cc(-c4ccccc4)cc(N(c4ccc5c(c4)oc4ccccc45)c4ccc5ccc6cc7oc8ccccc8c7cc6c5c4)c3)cc2)cc1 ; c1ccc(-c2ccc(N(c3ccccc3)c3cc(-c4ccccc4)cc(N(c4ccc5ccc6cc7oc8ccccc8c7cc6c5c4)c4ccc5oc6ccccc6c5c4)c3)cc2)cc1]

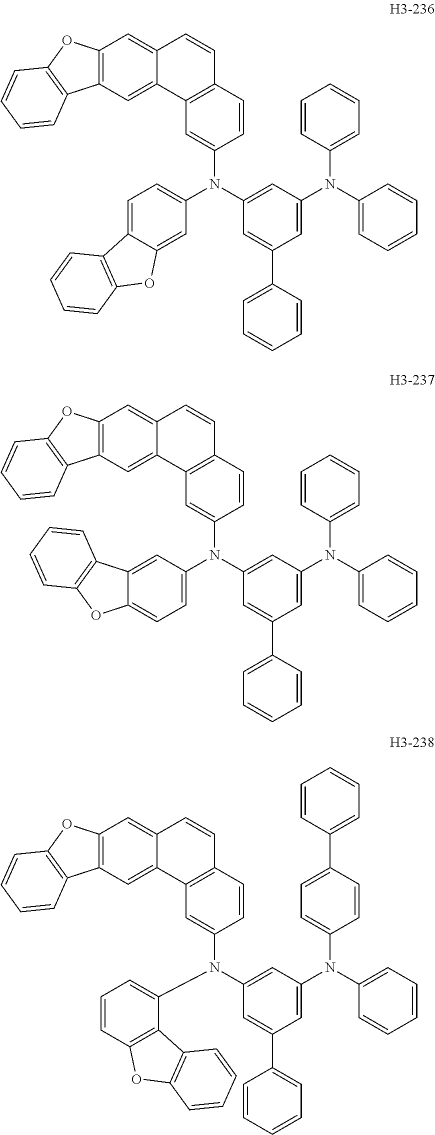 [SMILES: c1ccc(-c2cc(N(c3ccccc3)c3ccccc3)cc(N(c3ccc4c(c3)oc3ccccc34)c3ccc4ccc5cc6oc7ccccc7c6cc5c4c3)c2)cc1 ; c1ccc(-c2cc(N(c3ccccc3)c3ccccc3)cc(N(c3ccc4ccc5cc6oc7ccccc7c6cc5c4c3)c3ccc4oc5ccccc5c4c3)c2)cc1 ; c1ccc(-c2ccc(N(c3ccccc3)c3cc(-c4ccccc4)cc(N(c4ccc5ccc6cc7oc8ccccc8c7cc6c5c4)c4cccc5oc6ccccc6c45)c3)cc2)cc1]

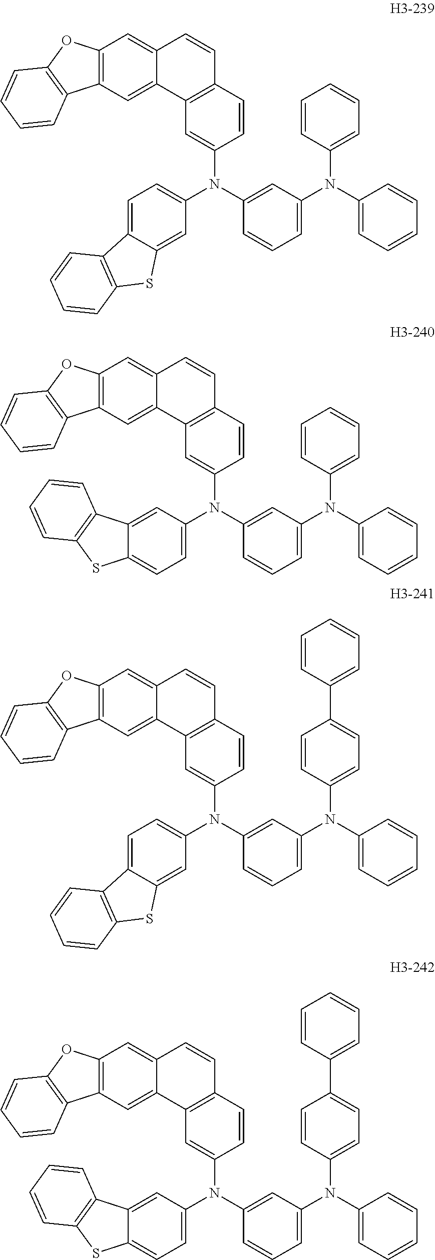 [SMILES: c1ccc(N(c2ccccc2)c2cccc(N(c3ccc4c(c3)sc3ccccc34)c3ccc4ccc5cc6oc7ccccc7c6cc5c4c3)c2)cc1 ; c1ccc(N(c2ccccc2)c2cccc(N(c3ccc4ccc5cc6oc7ccccc7c6cc5c4c3)c3ccc4sc5ccccc5c4c3)c2)cc1 ; c1ccc(-c2ccc(N(c3ccccc3)c3cccc(N(c4ccc5c(c4)sc4ccccc45)c4ccc5ccc6cc7oc8ccccc8c7cc6c5c4)c3)cc2)cc1 ; c1ccc(-c2ccc(N(c3ccccc3)c3cccc(N(c4ccc5ccc6cc7oc8ccccc8c7cc6c5c4)c4ccc5sc6ccccc6c5c4)c3)cc2)cc1]

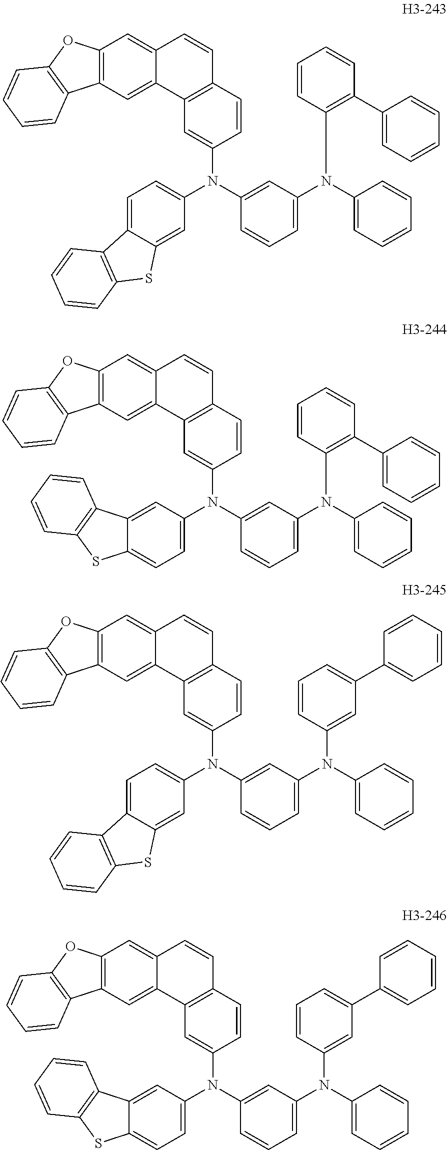 [SMILES: c1ccc(-c2ccccc2N(c2ccccc2)c2cccc(N(c3ccc4c(c3)sc3ccccc34)c3ccc4ccc5cc6oc7ccccc7c6cc5c4c3)c2)cc1 ; c1ccc(-c2ccccc2N(c2ccccc2)c2cccc(N(c3ccc4ccc5cc6oc7ccccc7c6cc5c4c3)c3ccc4sc5ccccc5c4c3)c2)cc1 ; c1ccc(-c2cccc(N(c3ccccc3)c3cccc(N(c4ccc5c(c4)sc4ccccc45)c4ccc5ccc6cc7oc8ccccc8c7cc6c5c4)c3)c2)cc1 ; c1ccc(-c2cccc(N(c3ccccc3)c3cccc(N(c4ccc5ccc6cc7oc8ccccc8c7cc6c5c4)c4ccc5sc6ccccc6c5c4)c3)c2)cc1]

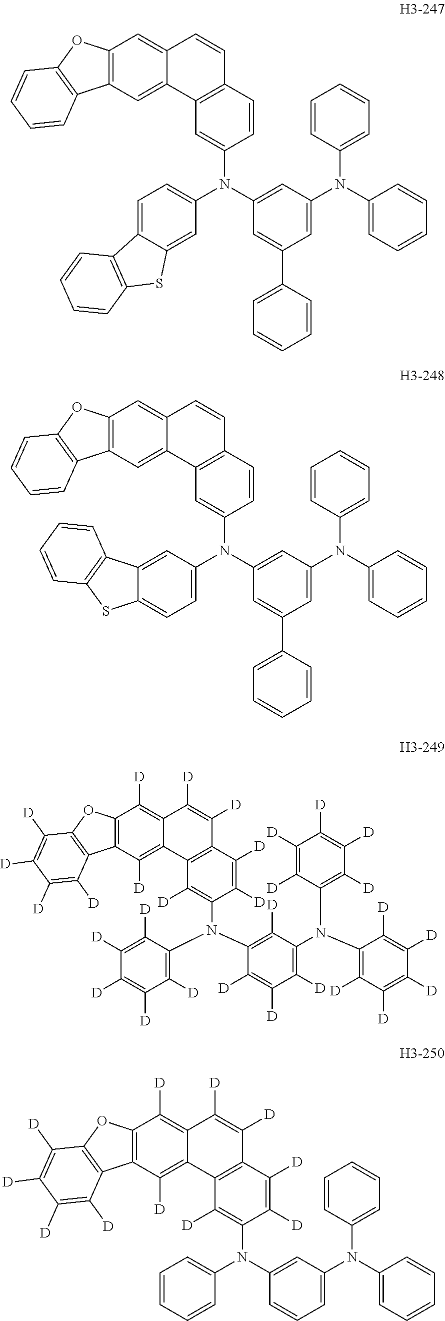

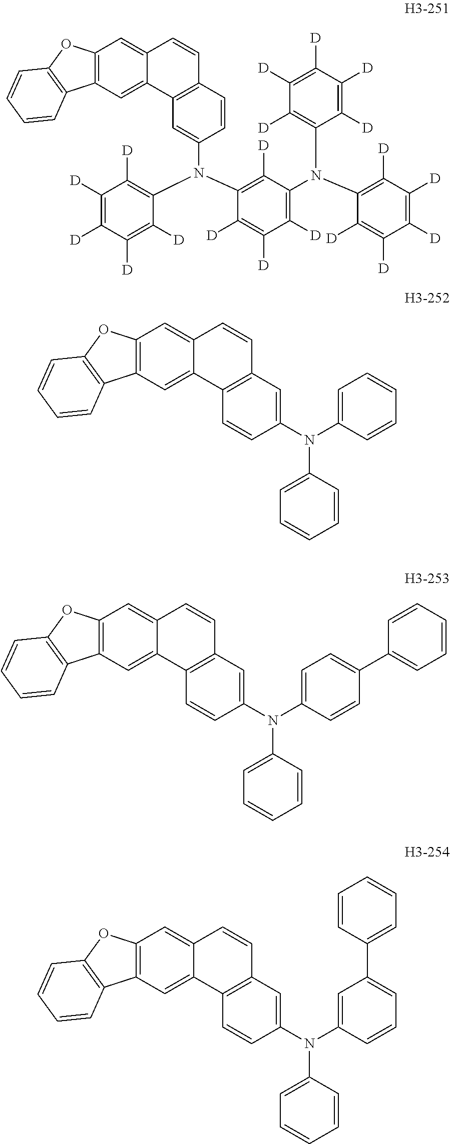 [SMILES: [2H]c1c([2H])c([2H])c(N(c2ccc3ccc4cc5oc6ccccc6c5cc4c3c2)c2c([2H])c([2H])c([2H])c(N(c3c([2H])c([2H])c([2H])c([2H])c3[2H])c3c([2H])c([2H])c([2H])c([2H])c3[2H])c2[2H])c([2H])c1[2H] ; c1ccc(N(c2ccccc2)c2ccc3c(ccc4cc5oc6ccccc6c5cc43)c2)cc1 ; c1ccc(-c2ccc(N(c3ccccc3)c3ccc4c(ccc5cc6oc7ccccc7c6cc54)c3)cc2)cc1 ; c1ccc(-c2cccc(N(c3ccccc3)c3ccc4c(ccc5cc6oc7ccccc7c6cc54)c3)c2)cc1]

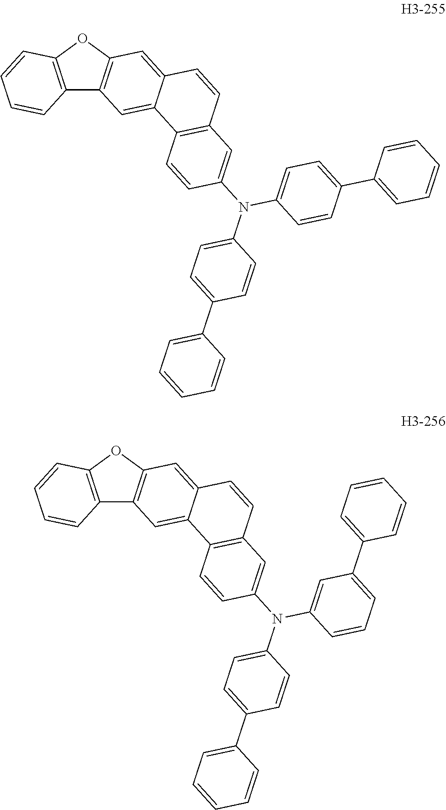 [SMILES: c1ccc(-c2ccc(N(c3ccc(-c4ccccc4)cc3)c3ccc4c(ccc5cc6oc7ccccc7c6cc54)c3)cc2)cc1 ; c1ccc(-c2ccc(N(c3cccc(-c4ccccc4)c3)c3ccc4c(ccc5cc6oc7ccccc7c6cc54)c3)cc2)cc1]

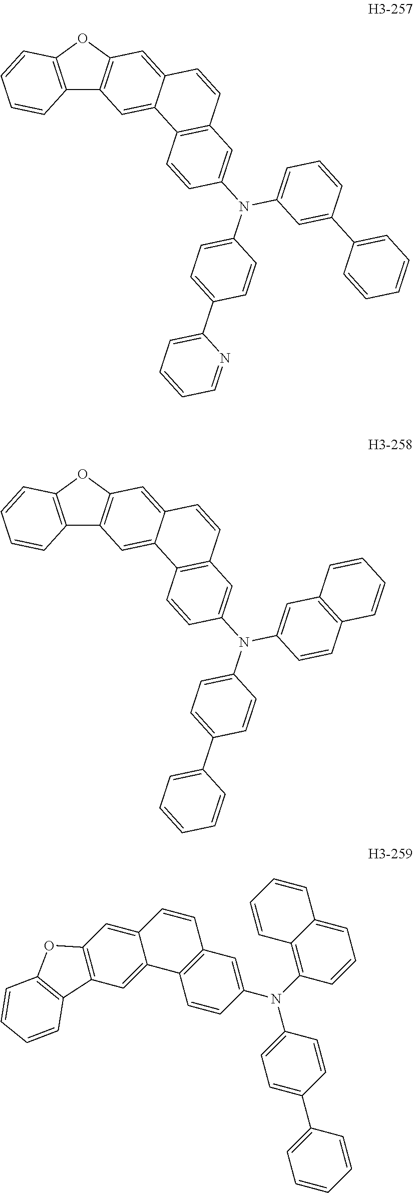 [SMILES: c1ccc(-c2cccc(N(c3ccc(-c4ccccn4)cc3)c3ccc4c(ccc5cc6oc7ccccc7c6cc54)c3)c2)cc1 ; c1ccc(-c2ccc(N(c3ccc4ccccc4c3)c3ccc4c(ccc5cc6oc7ccccc7c6cc54)c3)cc2)cc1 ; c1ccc(-c2ccc(N(c3ccc4c(ccc5cc6oc7ccccc7c6cc54)c3)c3cccc4ccccc34)cc2)cc1]

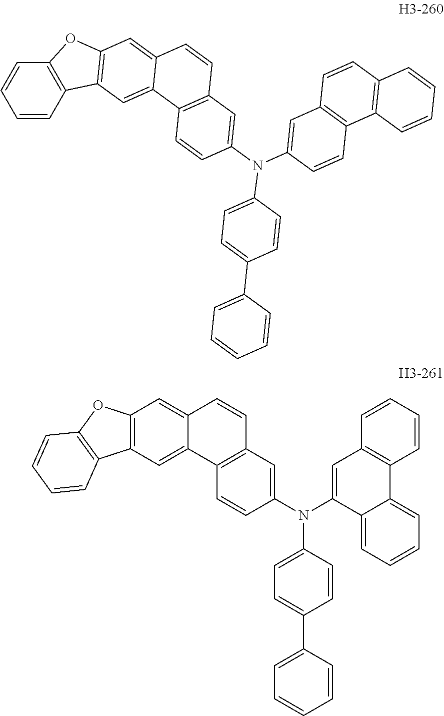

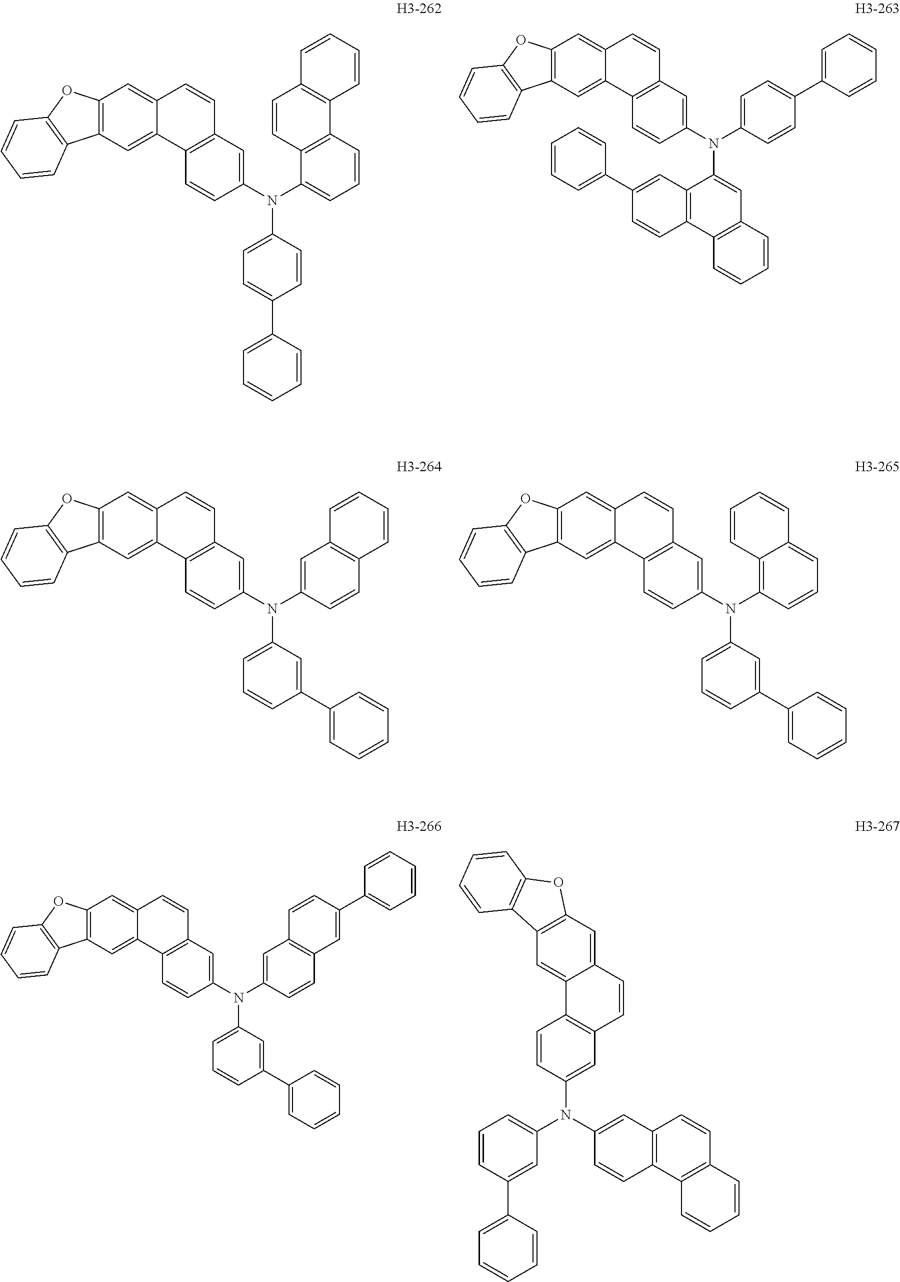

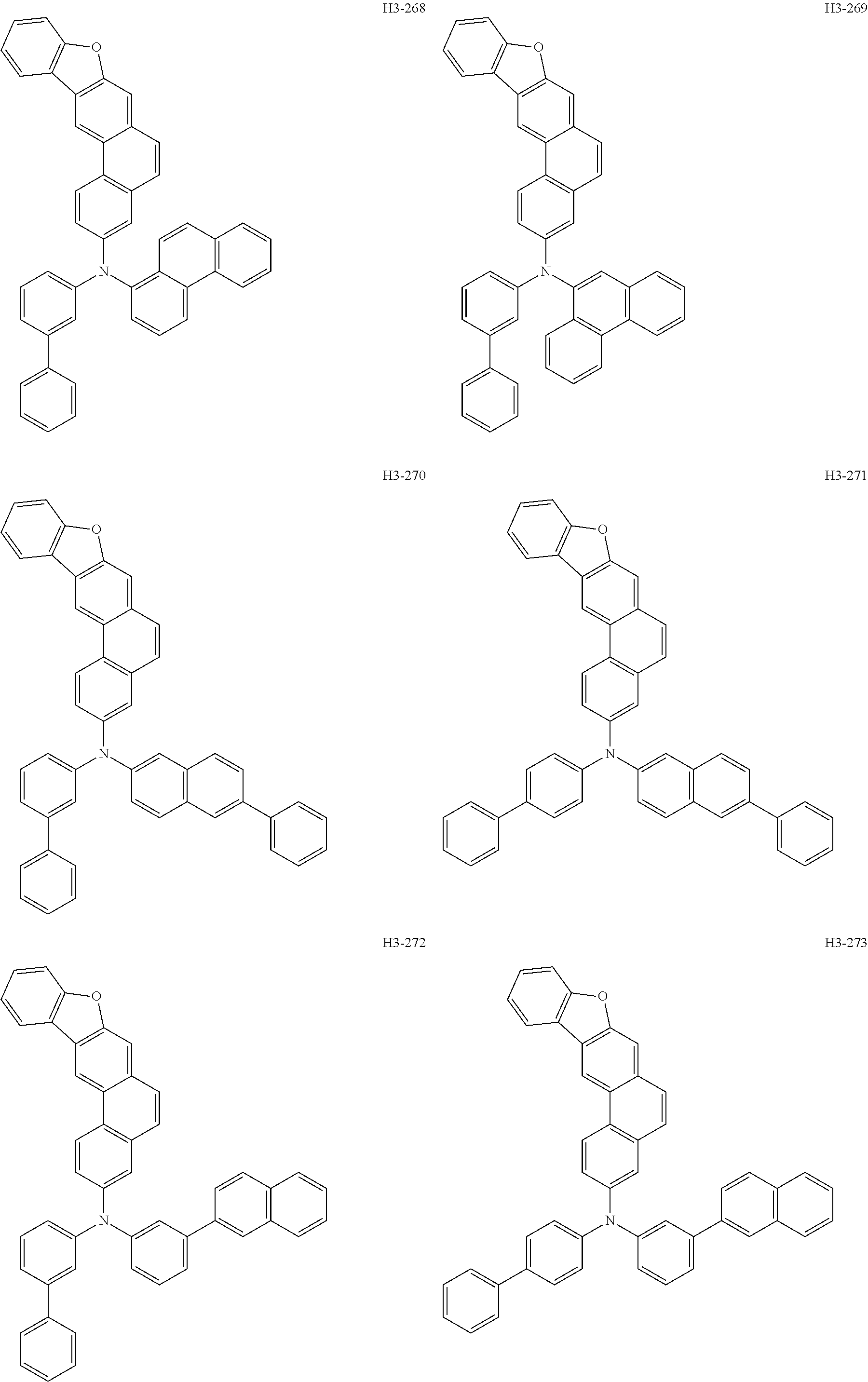 [SMILES: c1ccc(-c2cccc(N(c3ccc4c(ccc5cc6oc7ccccc7c6cc54)c3)c3cccc4c3ccc3ccccc34)c2)cc1 ; c1ccc(-c2cccc(N(c3ccc4c(ccc5cc6oc7ccccc7c6cc54)c3)c3cc4ccccc4c4ccccc34)c2)cc1 ; c1ccc(-c2cccc(N(c3ccc4cc(-c5ccccc5)ccc4c3)c3ccc4c(ccc5cc6oc7ccccc7c6cc54)c3)c2)cc1 ; c1ccc(-c2ccc(N(c3ccc4cc(-c5ccccc5)ccc4c3)c3ccc4c(ccc5cc6oc7ccccc7c6cc54)c3)cc2)cc1 ; c1ccc(-c2cccc(N(c3cccc(-c4ccc5ccccc5c4)c3)c3ccc4c(ccc5cc6oc7ccccc7c6cc54)c3)c2)cc1 ; c1ccc(-c2ccc(N(c3cccc(-c4ccc5ccccc5c4)c3)c3ccc4c(ccc5cc6oc7ccccc7c6cc54)c3)cc2)cc1]

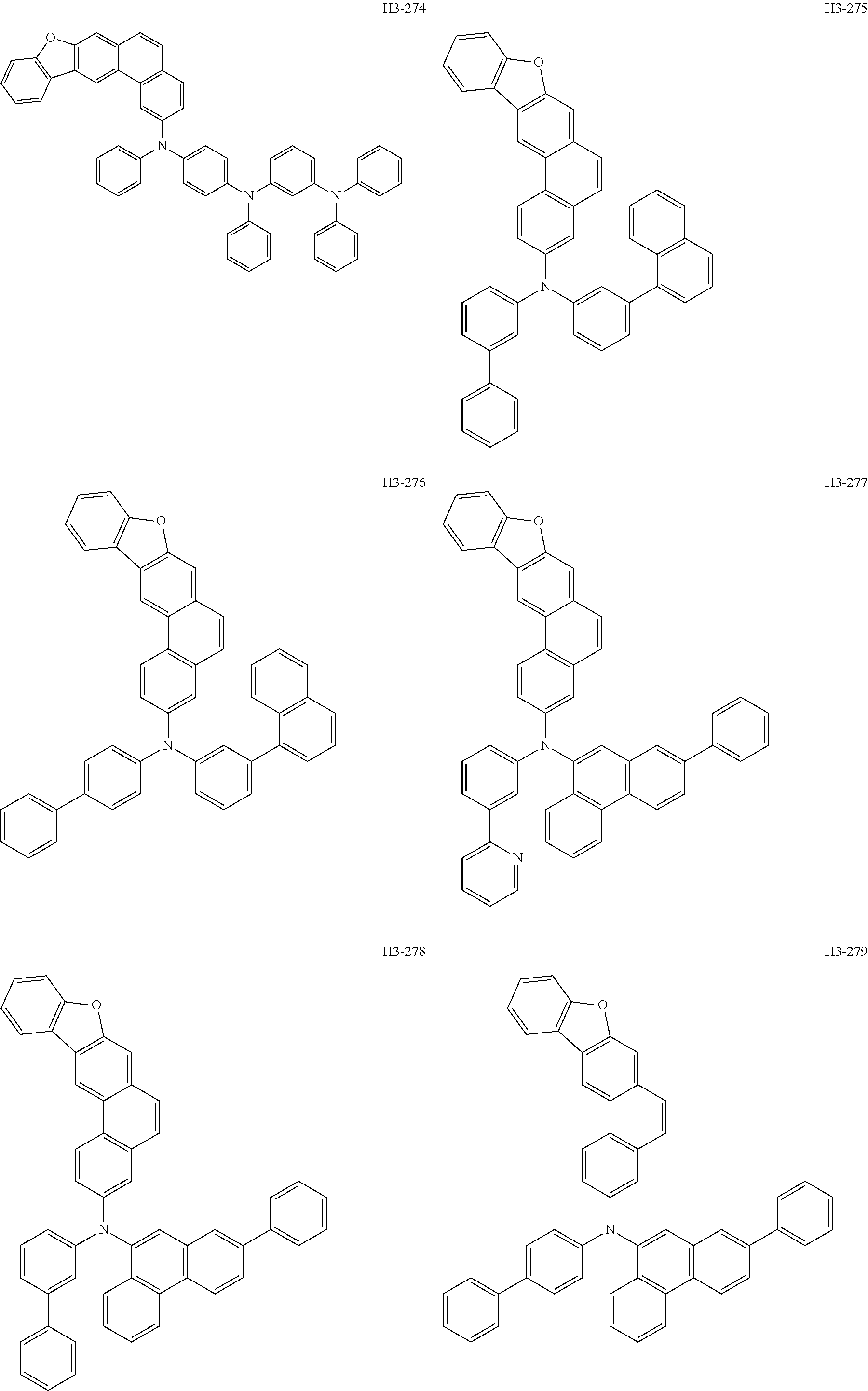

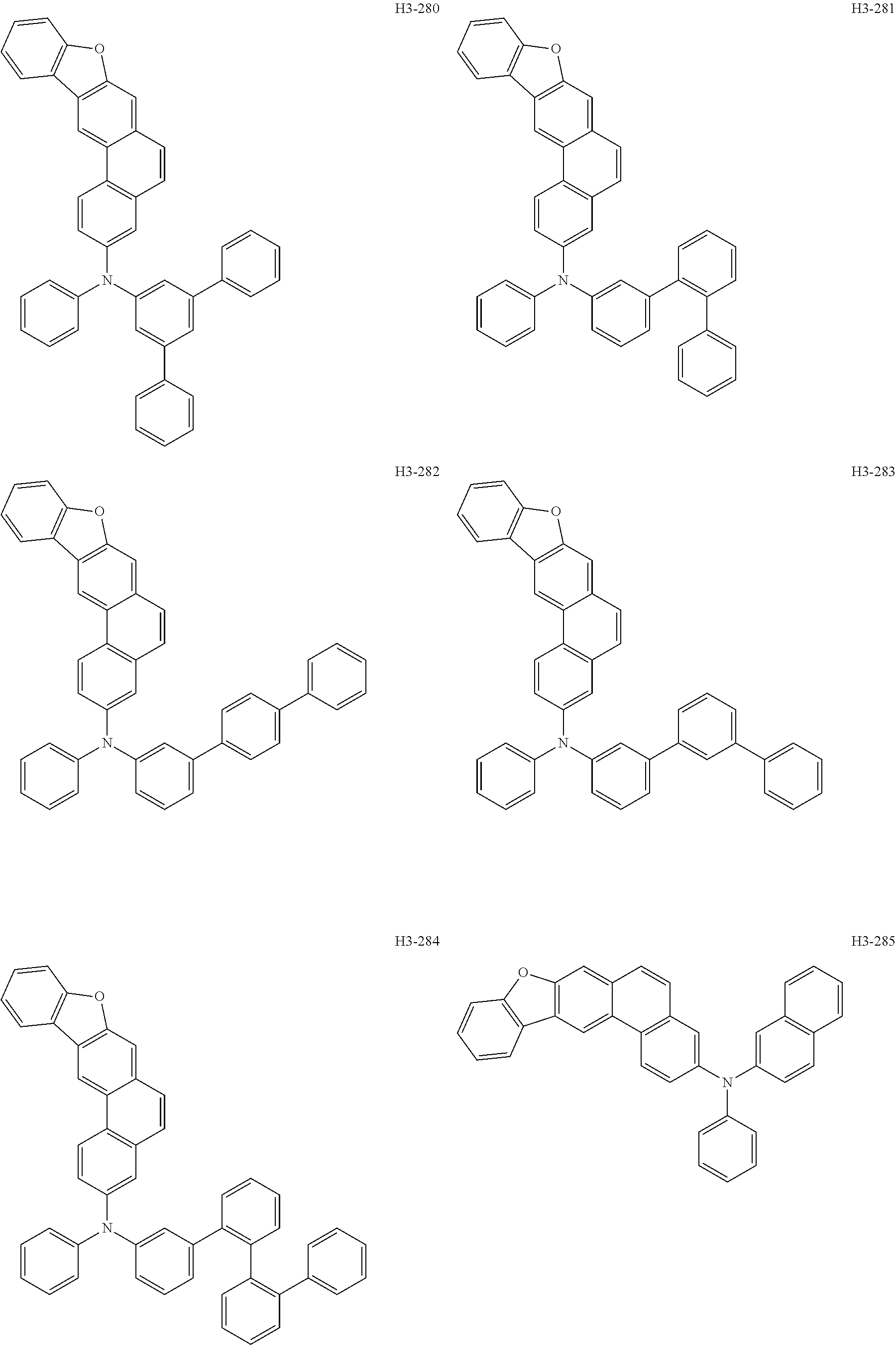 [SMILES: c1ccc(-c2cc(-c3ccccc3)cc(N(c3ccccc3)c3ccc4c(ccc5cc6oc7ccccc7c6cc54)c3)c2)cc1 ; c1ccc(-c2ccccc2-c2cccc(N(c3ccccc3)c3ccc4c(ccc5cc6oc7ccccc7c6cc54)c3)c2)cc1 ; c1ccc(-c2ccc(-c3cccc(N(c4ccccc4)c4ccc5c(ccc6cc7oc8ccccc8c7cc65)c4)c3)cc2)cc1 ; c1ccc(-c2cccc(-c3cccc(N(c4ccccc4)c4ccc5c(ccc6cc7oc8ccccc8c7cc65)c4)c3)c2)cc1 ; c1ccc(-c2ccccc2-c2ccccc2-c2cccc(N(c3ccccc3)c3ccc4c(ccc5cc6oc7ccccc7c6cc54)c3)c2)cc1 ; c1ccc(N(c2ccc3ccccc3c2)c2ccc3c(ccc4cc5oc6ccccc6c5cc43)c2)cc1]

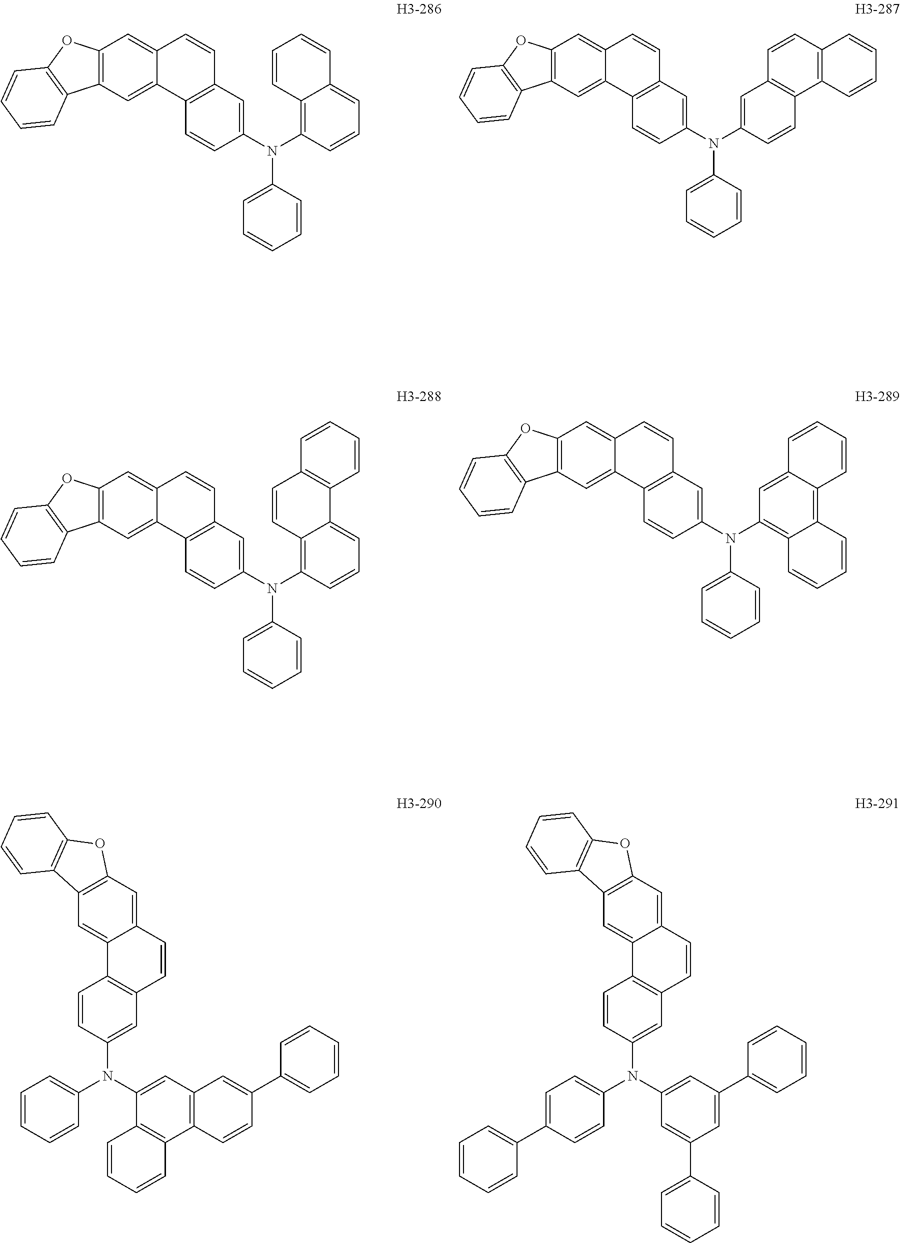 [SMILES: c1ccc(N(c2ccc3c(ccc4cc5oc6ccccc6c5cc43)c2)c2cccc3ccccc23)cc1 ; c1ccc(N(c2ccc3c(ccc4ccccc43)c2)c2ccc3c(ccc4cc5oc6ccccc6c5cc43)c2)cc1 ; c1ccc(N(c2ccc3c(ccc4cc5oc6ccccc6c5cc43)c2)c2cccc3c2ccc2ccccc23)cc1 ; c1ccc(N(c2ccc3c(ccc4cc5oc6ccccc6c5cc43)c2)c2cc3ccccc3c3ccccc23)cc1 ; c1ccc(-c2ccc3c(c2)cc(N(c2ccccc2)c2ccc4c(ccc5cc6oc7ccccc7c6cc54)c2)c2ccccc23)cc1 ; c1ccc(-c2ccc(N(c3cc(-c4ccccc4)cc(-c4ccccc4)c3)c3ccc4c(ccc5cc6oc7ccccc7c6cc54)c3)cc2)cc1]

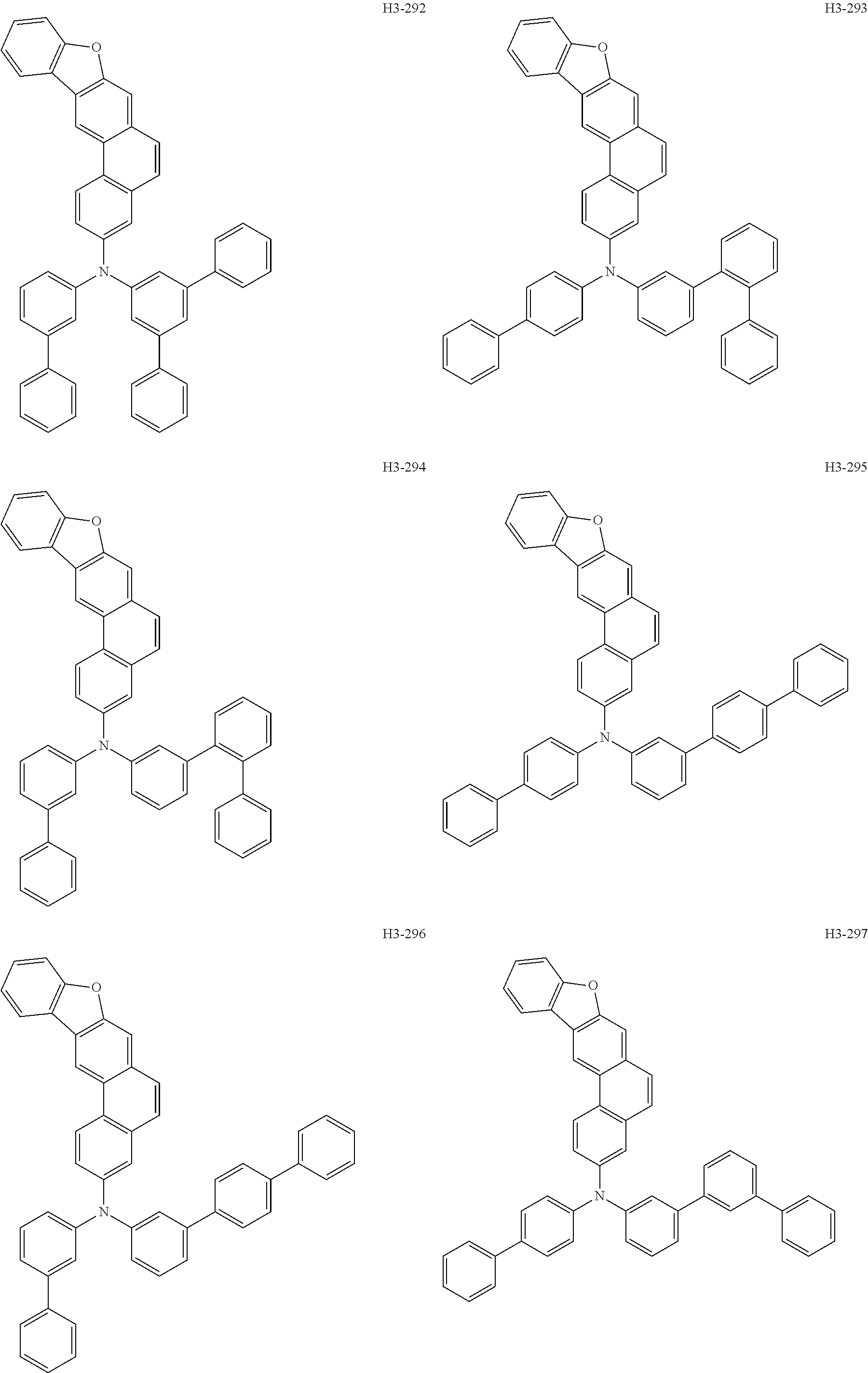

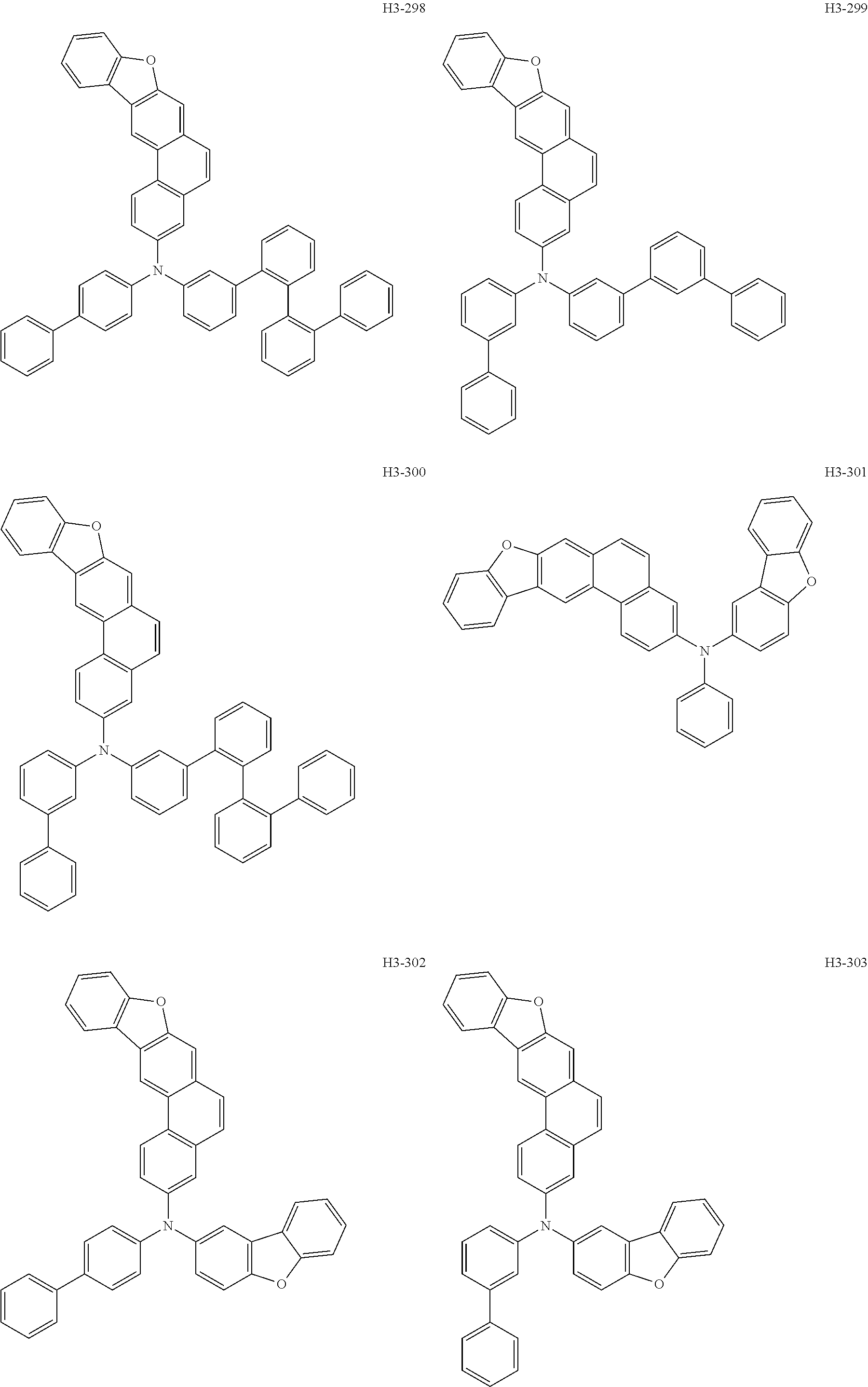

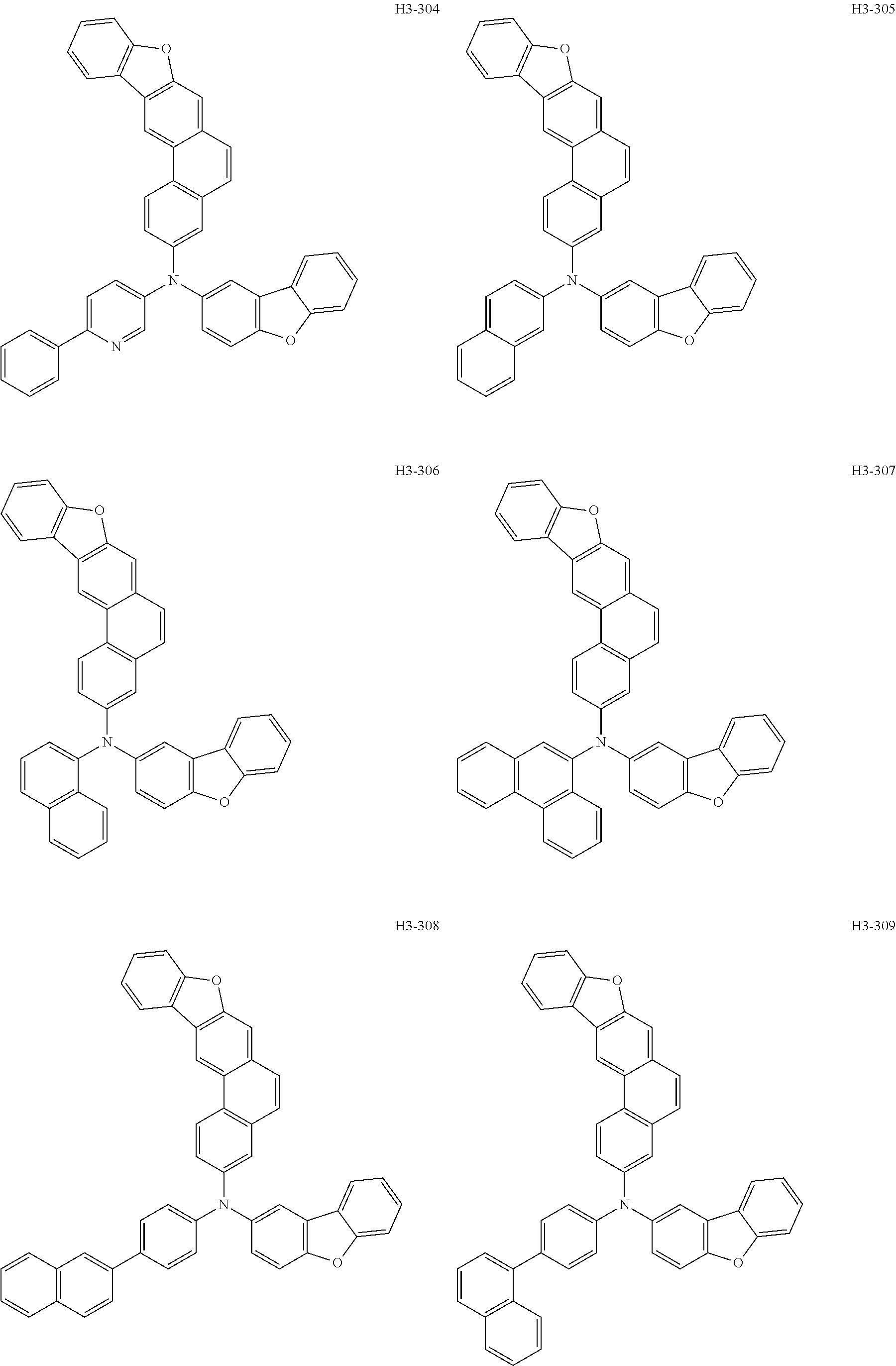 [SMILES: c1ccc(-c2ccc(N(c3ccc4c(ccc5cc6oc7ccccc7c6cc54)c3)c3ccc4oc5ccccc5c4c3)cn2)cc1 ; c1ccc2cc(N(c3ccc4c(ccc5cc6oc7ccccc7c6cc54)c3)c3ccc4oc5ccccc5c4c3)ccc2c1 ; c1ccc2c(N(c3ccc4c(ccc5cc6oc7ccccc7c6cc54)c3)c3ccc4oc5ccccc5c4c3)cccc2c1 ; c1ccc2c(c1)cc(N(c1ccc3c(ccc4cc5oc6ccccc6c5cc43)c1)c1ccc3oc4ccccc4c3c1)c1ccccc12 ; c1ccc2cc(-c3ccc(N(c4ccc5c(ccc6cc7oc8ccccc8c7cc65)c4)c4ccc5oc6ccccc6c5c4)cc3)ccc2c1 ; c1ccc2c(-c3ccc(N(c4ccc5c(ccc6cc7oc8ccccc8c7cc65)c4)c4ccc5oc6ccccc6c5c4)cc3)cccc2c1]

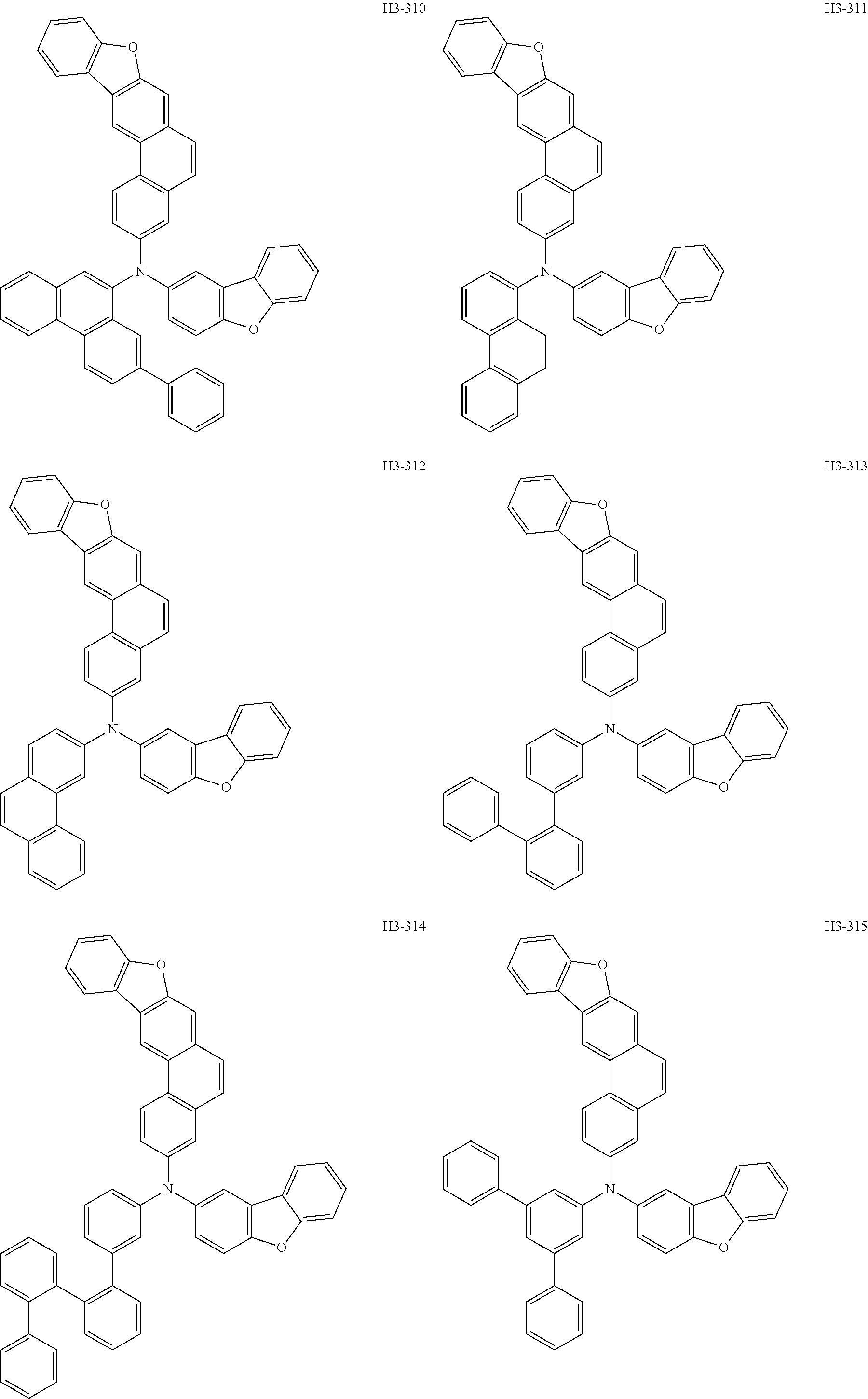

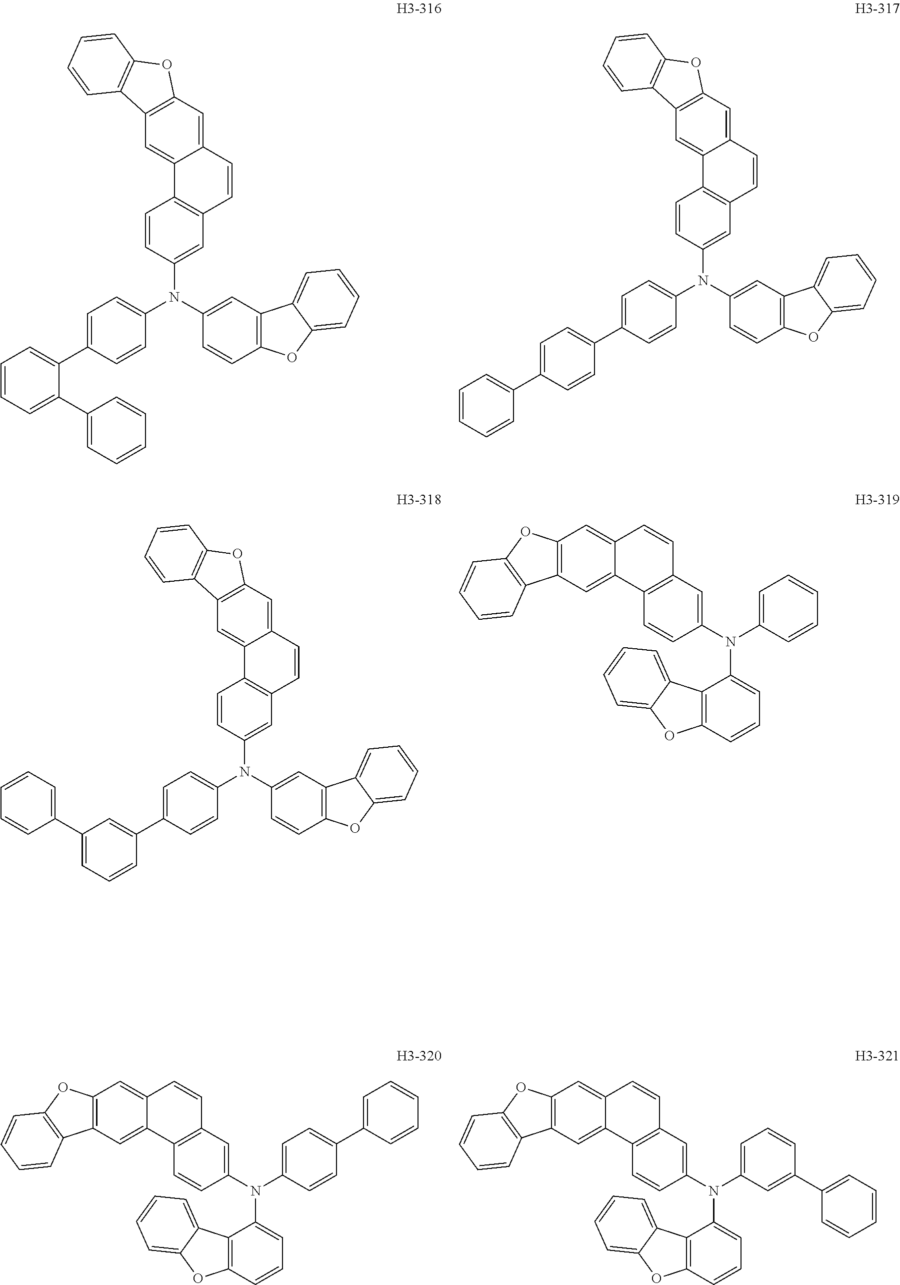 [SMILES: c1ccc(-c2ccccc2-c2ccc(N(c3ccc4c(ccc5cc6oc7ccccc7c6cc54)c3)c3ccc4oc5ccccc5c4c3)cc2)cc1 ; c1ccc(-c2ccc(-c3ccc(N(c4ccc5c(ccc6cc7oc8ccccc8c7cc65)c4)c4ccc5oc6ccccc6c5c4)cc3)cc2)cc1 ; c1ccc(-c2cccc(-c3ccc(N(c4ccc5c(ccc6cc7oc8ccccc8c7cc65)c4)c4ccc5oc6ccccc6c5c4)cc3)c2)cc1 ; c1ccc(N(c2ccc3c(ccc4cc5oc6ccccc6c5cc43)c2)c2cccc3oc4ccccc4c23)cc1 ; c1ccc(-c2ccc(N(c3ccc4c(ccc5cc6oc7ccccc7c6cc54)c3)c3cccc4oc5ccccc5c34)cc2)cc1 ; c1ccc(-c2cccc(N(c3ccc4c(ccc5cc6oc7ccccc7c6cc54)c3)c3cccc4oc5ccccc5c34)c2)cc1]

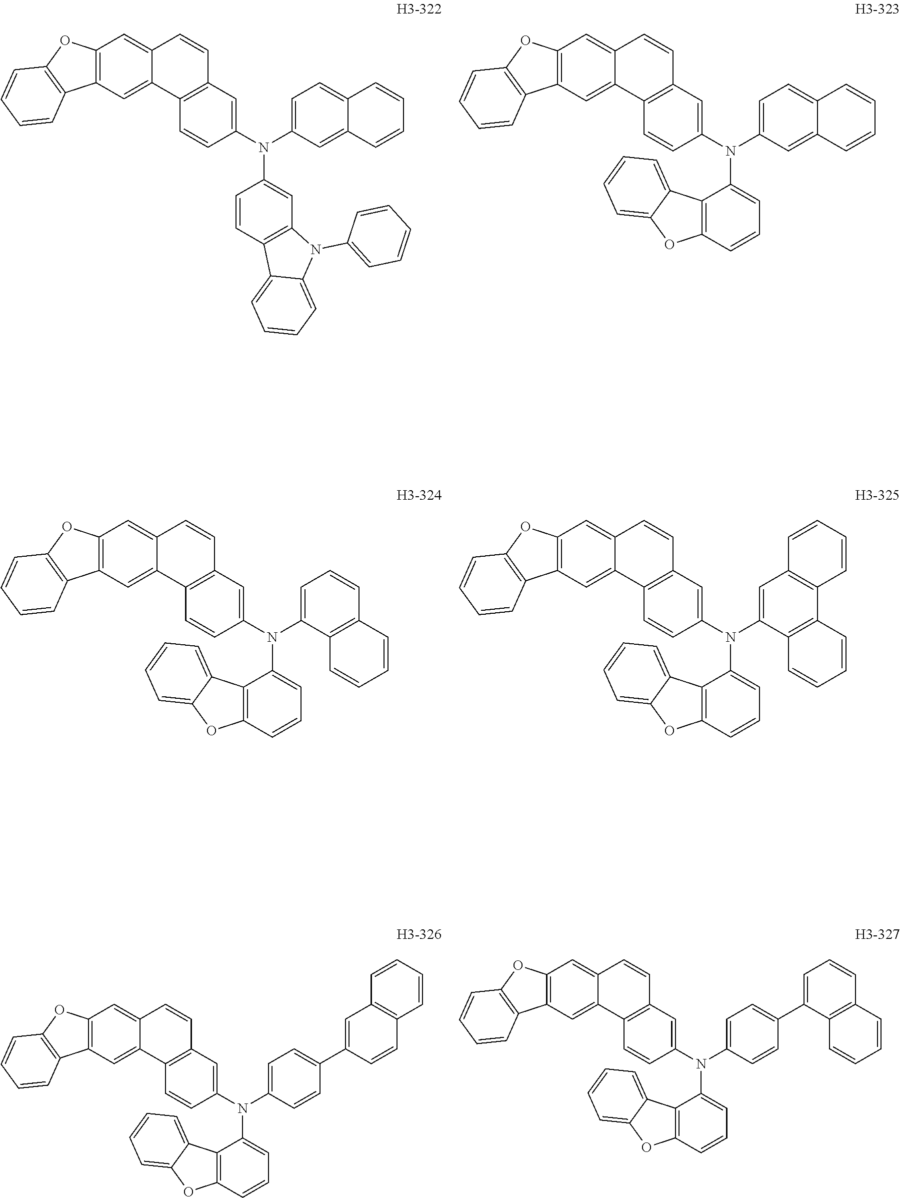

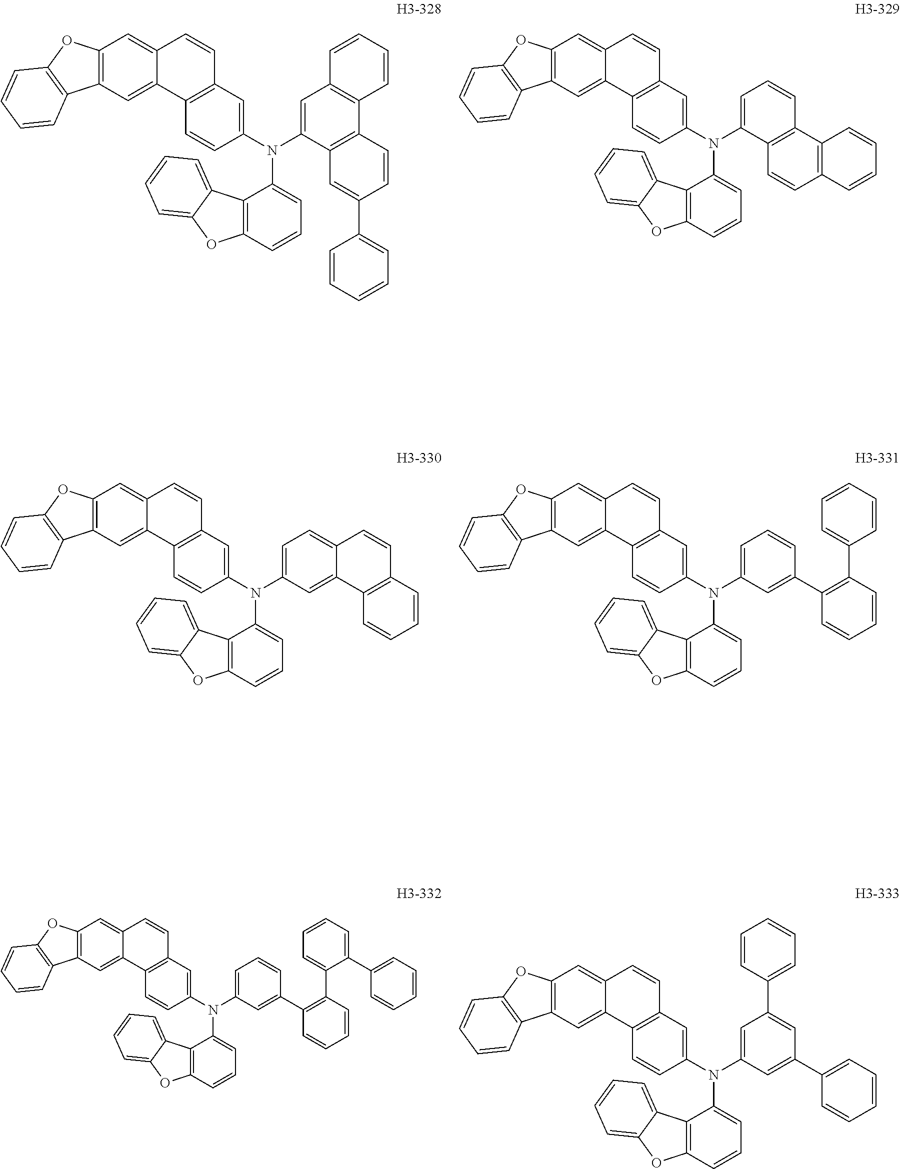

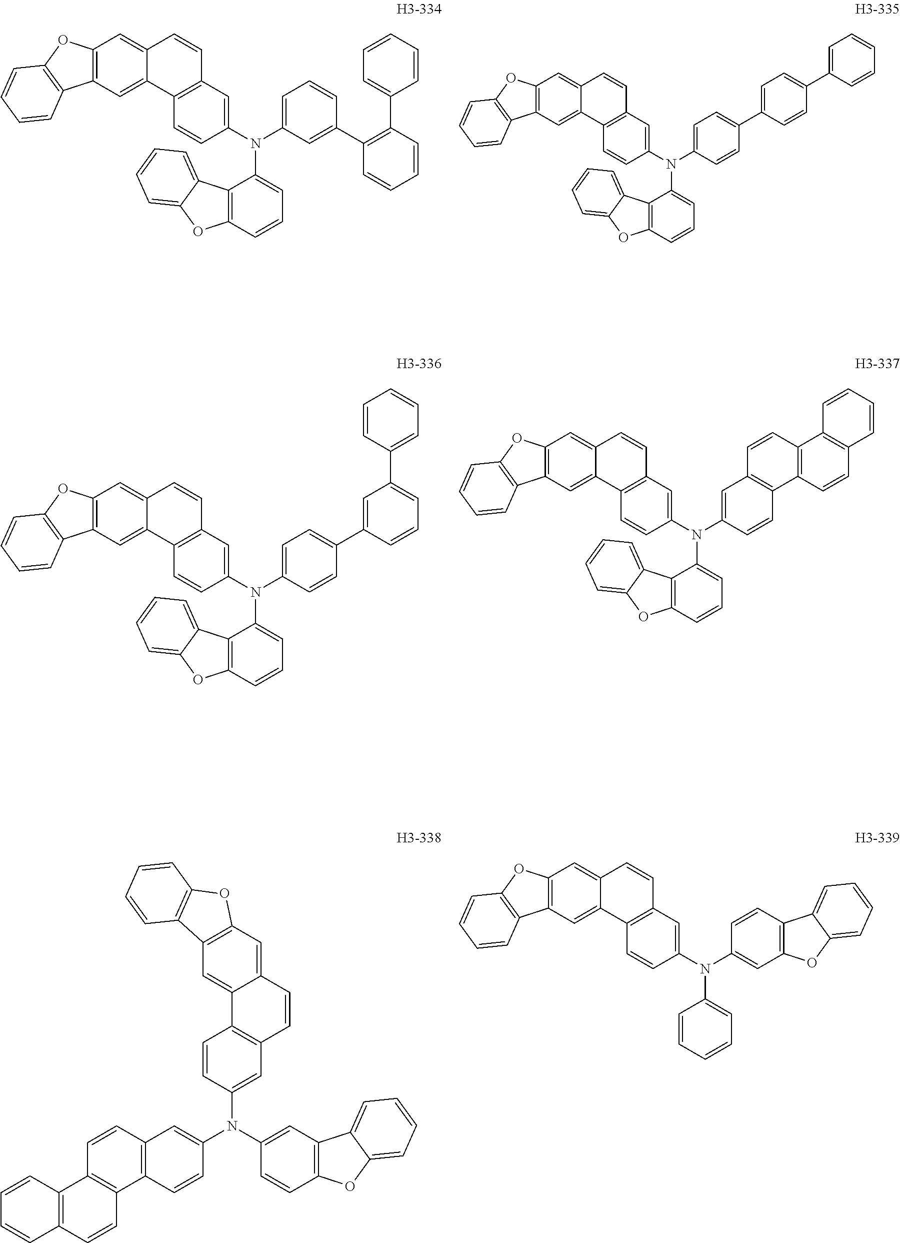

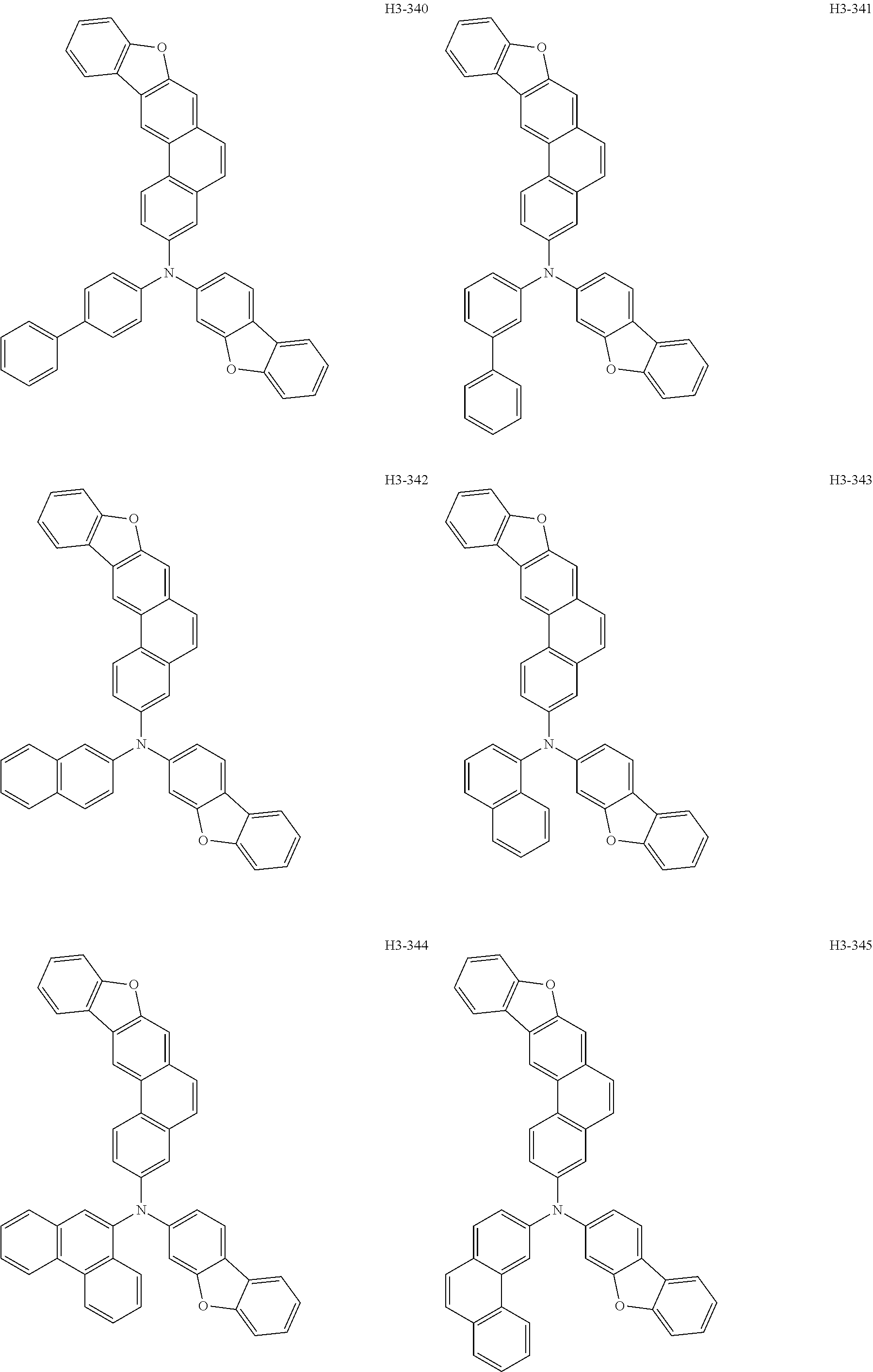 [SMILES: c1ccc(-c2ccc(N(c3ccc4c(ccc5cc6oc7ccccc7c6cc54)c3)c3ccc4c(c3)oc3ccccc34)cc2)cc1 ; c1ccc(-c2cccc(N(c3ccc4c(ccc5cc6oc7ccccc7c6cc54)c3)c3ccc4c(c3)oc3ccccc34)c2)cc1 ; c1ccc2cc(N(c3ccc4c(ccc5cc6oc7ccccc7c6cc54)c3)c3ccc4c(c3)oc3ccccc34)ccc2c1 ; c1ccc2c(N(c3ccc4c(ccc5cc6oc7ccccc7c6cc54)c3)c3ccc4c(c3)oc3ccccc34)cccc2c1 ; c1ccc2c(c1)cc(N(c1ccc3c(ccc4cc5oc6ccccc6c5cc43)c1)c1ccc3c(c1)oc1ccccc13)c1ccccc12 ; c1ccc2c(c1)ccc1ccc(N(c3ccc4c(ccc5cc6oc7ccccc7c6cc54)c3)c3ccc4c(c3)oc3ccccc34)cc12]

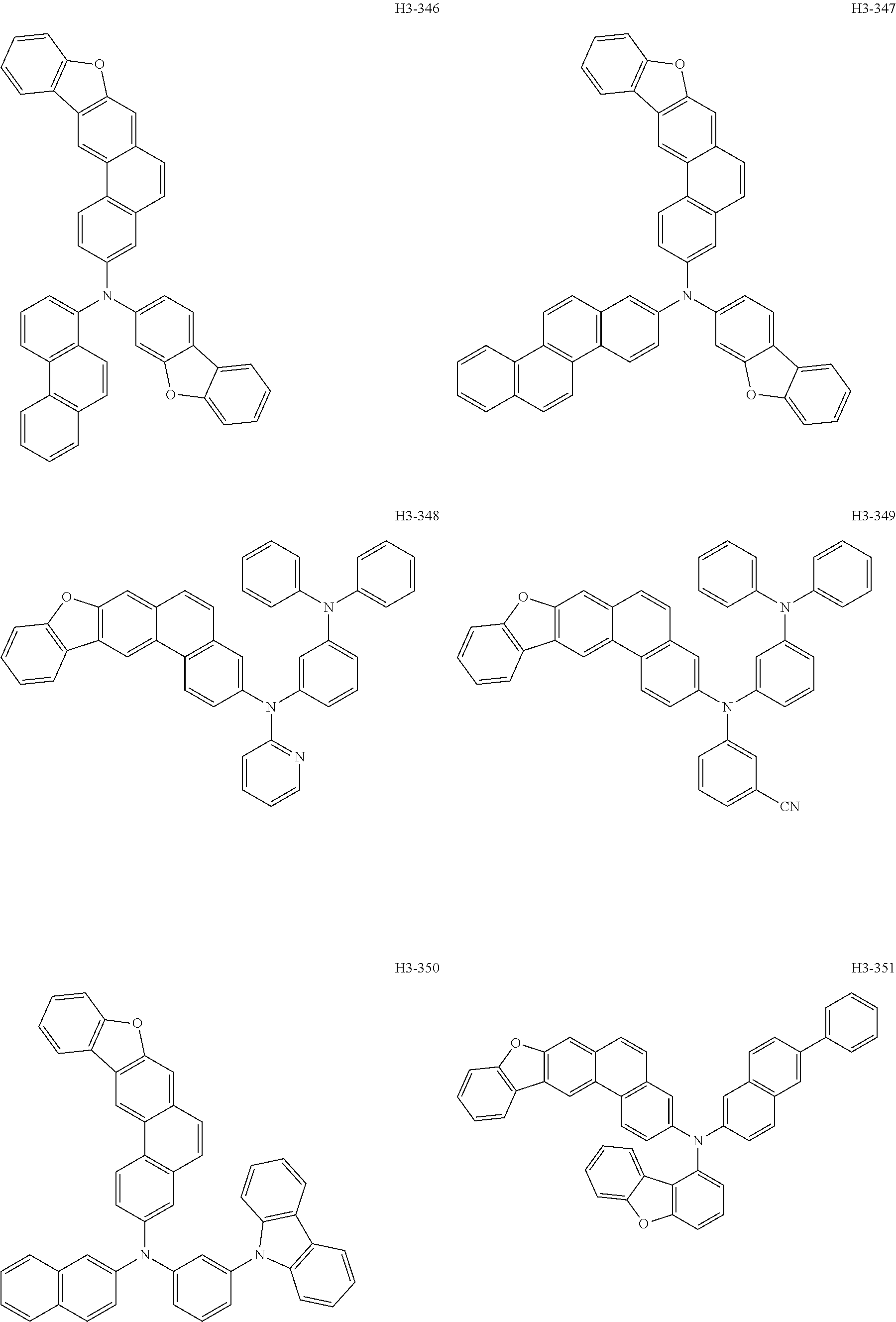 [SMILES: c1ccc2c(c1)ccc1c(N(c3ccc4c(ccc5cc6oc7ccccc7c6cc54)c3)c3ccc4c(c3)oc3ccccc34)cccc12 ; c1ccc2c(c1)ccc1c3ccc(N(c4ccc5c(ccc6cc7oc8ccccc8c7cc65)c4)c4ccc5c(c4)oc4ccccc45)cc3ccc21 ; c1ccc(N(c2ccccc2)c2cccc(N(c3ccc4c(ccc5cc6oc7ccccc7c6cc54)c3)c3ccccn3)c2)cc1 ; N#Cc1cccc(N(c2cccc(N(c3ccccc3)c3ccccc3)c2)c2ccc3c(ccc4cc5oc6ccccc6c5cc43)c2)c1 ; c1cc(N(c2ccc3ccccc3c2)c2ccc3c(ccc4cc5oc6ccccc6c5cc43)c2)cc(-n2c3ccccc3c3ccccc32)c1 ; c1ccc(-c2ccc3cc(N(c4ccc5c(ccc6cc7oc8ccccc8c7cc65)c4)c4cccc5oc6ccccc6c45)ccc3c2)cc1]

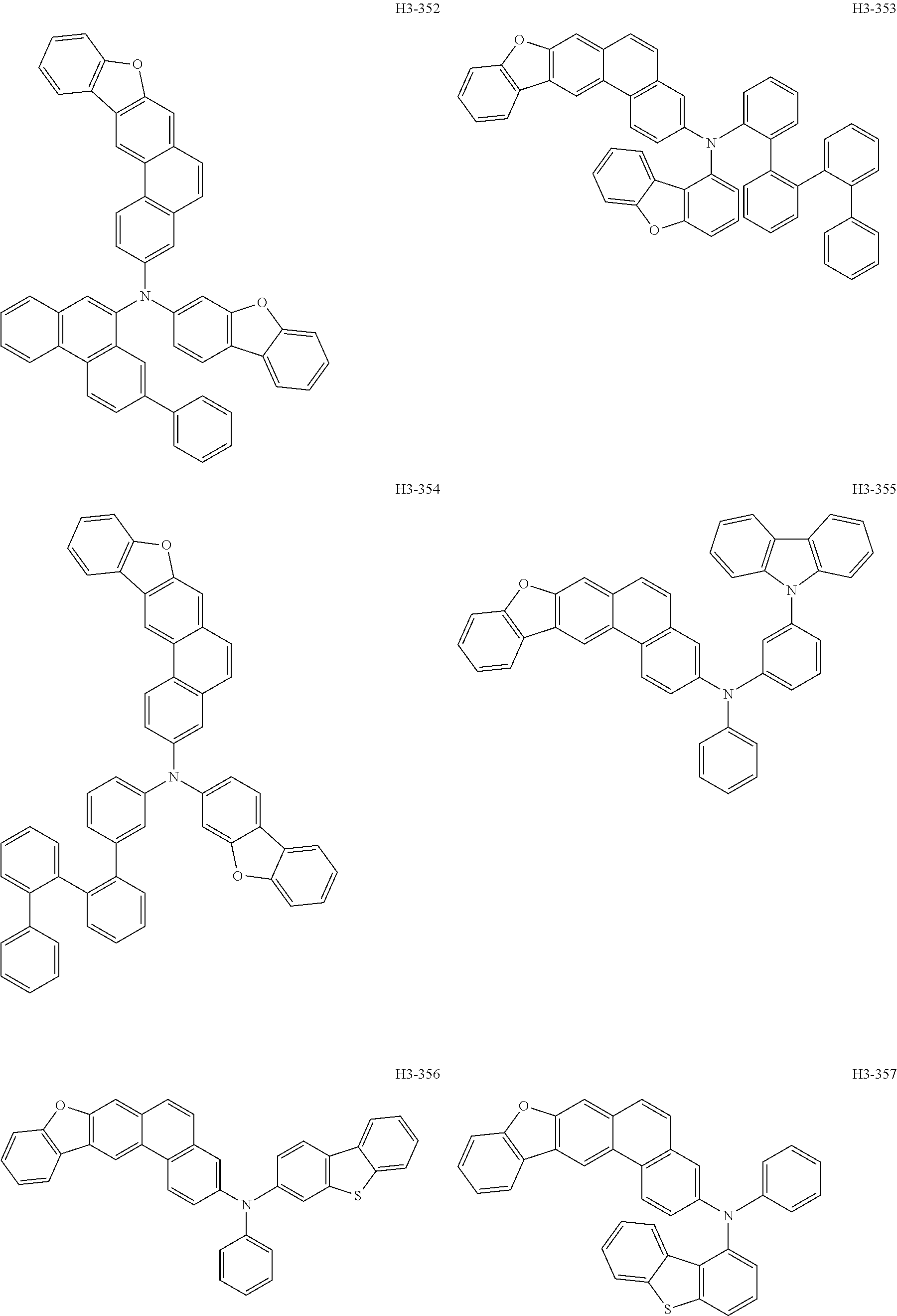

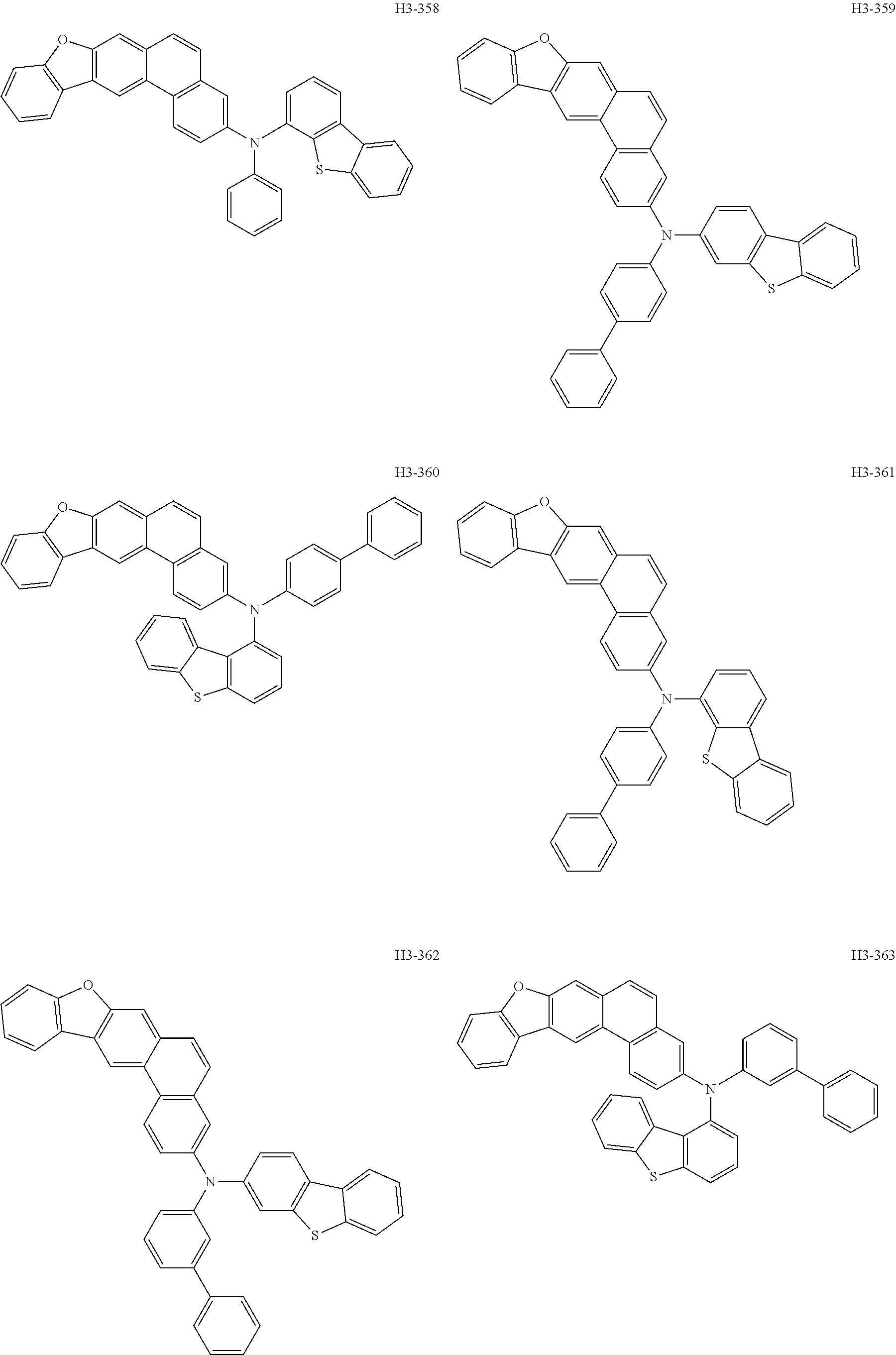

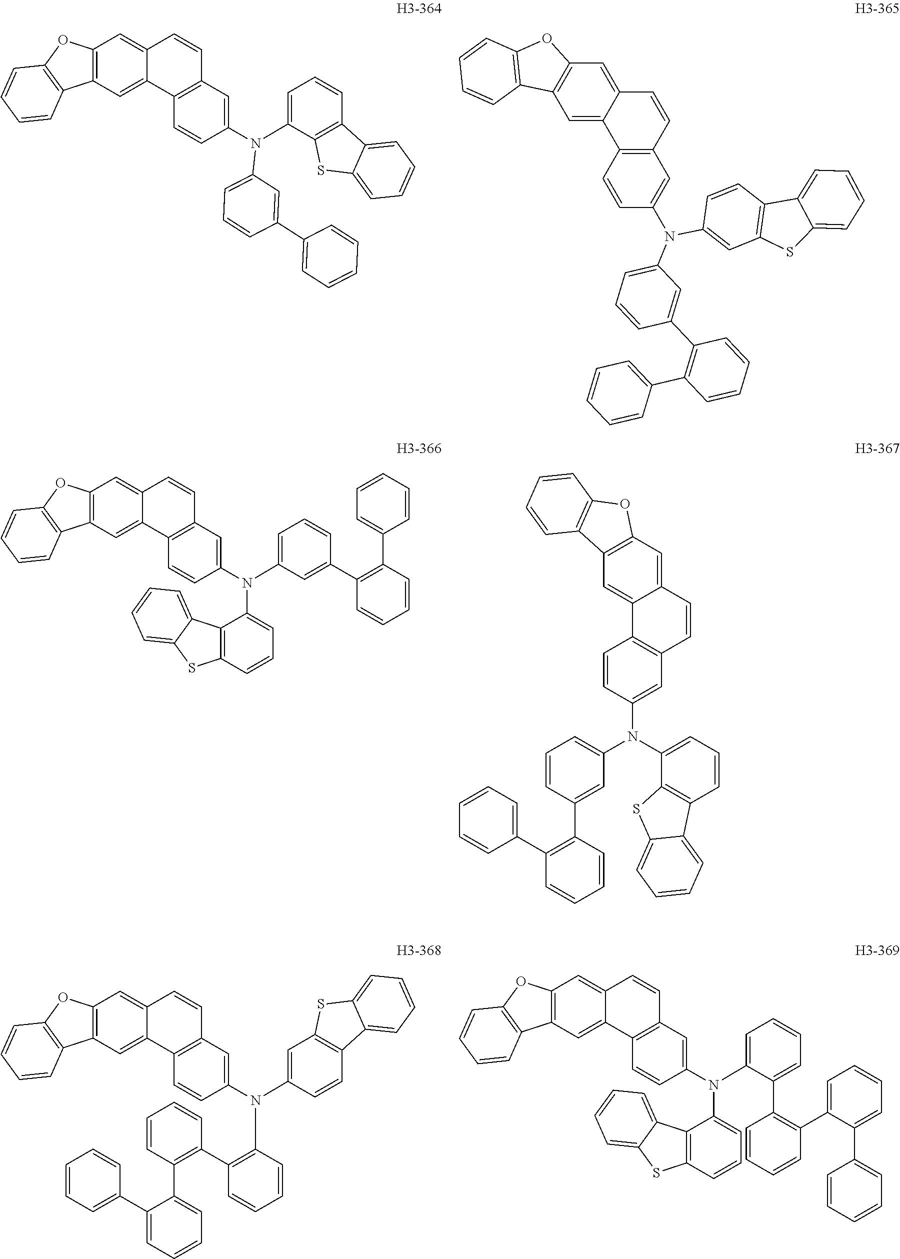

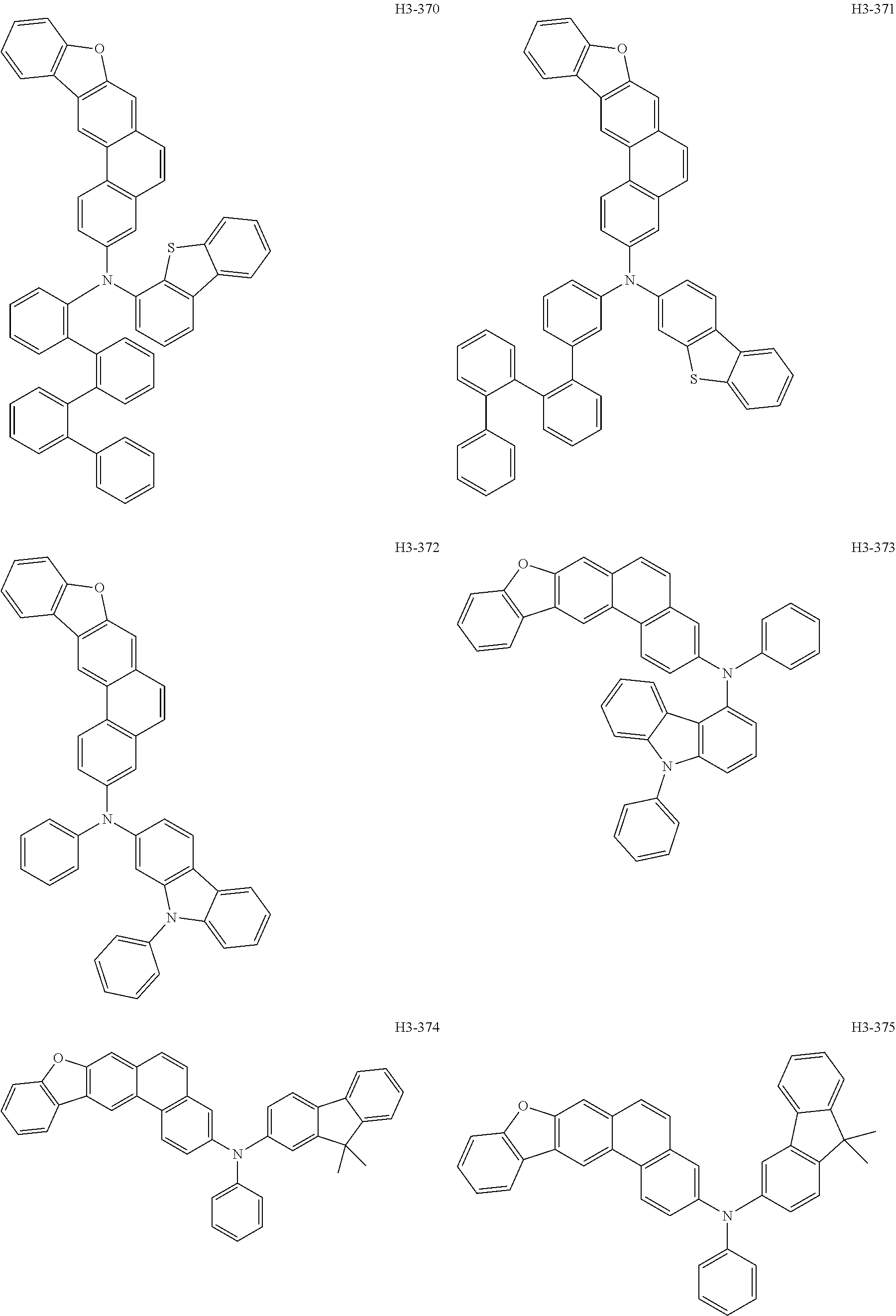 [SMILES: c1ccc(-c2ccccc2-c2ccccc2-c2ccccc2N(c2ccc3c(ccc4cc5oc6ccccc6c5cc43)c2)c2cccc3c2sc2ccccc23)cc1 ; c1ccc(-c2ccccc2-c2ccccc2-c2cccc(N(c3ccc4c(ccc5cc6oc7ccccc7c6cc54)c3)c3ccc4c(c3)sc3ccccc34)c2)cc1 ; c1ccc(N(c2ccc3c(ccc4cc5oc6ccccc6c5cc43)c2)c2ccc3c4ccccc4n(-c4ccccc4)c3c2)cc1 ; c1ccc(N(c2ccc3c(ccc4cc5oc6ccccc6c5cc43)c2)c2cccc3c2c2ccccc2n3-c2ccccc2)cc1 ; CC1(C)c2ccccc2-c2ccc(N(c3ccccc3)c3ccc4c(ccc5cc6oc7ccccc7c6cc54)c3)cc21 ; CC1(C)c2ccccc2-c2cc(N(c3ccccc3)c3ccc4c(ccc5cc6oc7ccccc7c6cc54)c3)ccc21]

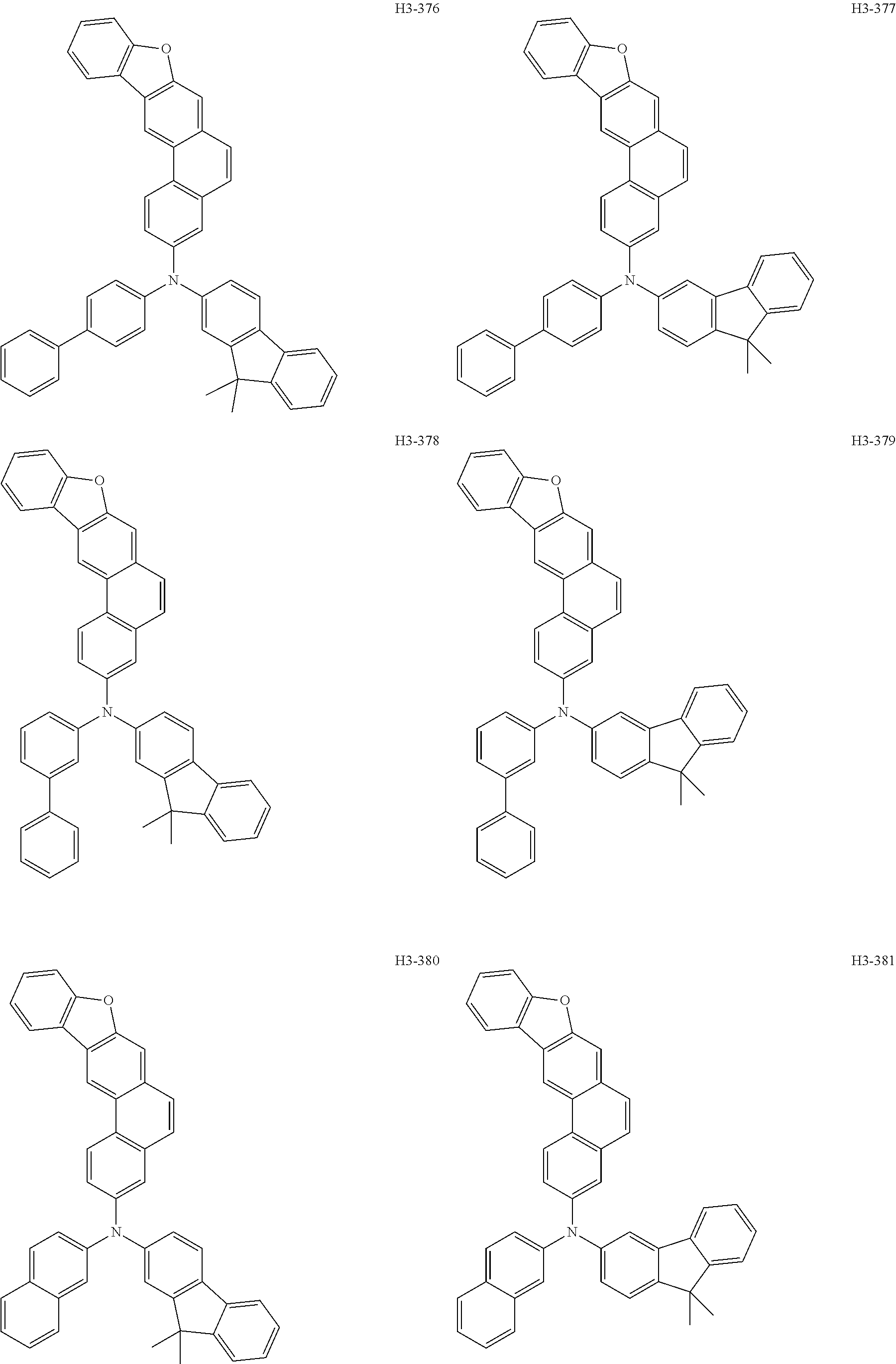 [SMILES: CC1(C)c2ccccc2-c2ccc(N(c3ccc(-c4ccccc4)cc3)c3ccc4c(ccc5cc6oc7ccccc7c6cc54)c3)cc21 ; CC1(C)c2ccccc2-c2cc(N(c3ccc(-c4ccccc4)cc3)c3ccc4c(ccc5cc6oc7ccccc7c6cc54)c3)ccc21 ; CC1(C)c2ccccc2-c2ccc(N(c3cccc(-c4ccccc4)c3)c3ccc4c(ccc5cc6oc7ccccc7c6cc54)c3)cc21 ; CC1(C)c2ccccc2-c2cc(N(c3cccc(-c4ccccc4)c3)c3ccc4c(ccc5cc6oc7ccccc7c6cc54)c3)ccc21 ; CC1(C)c2ccccc2-c2ccc(N(c3ccc4ccccc4c3)c3ccc4c(ccc5cc6oc7ccccc7c6cc54)c3)cc21 ; CC1(C)c2ccccc2-c2cc(N(c3ccc4ccccc4c3)c3ccc4c(ccc5cc6oc7ccccc7c6cc54)c3)ccc21]

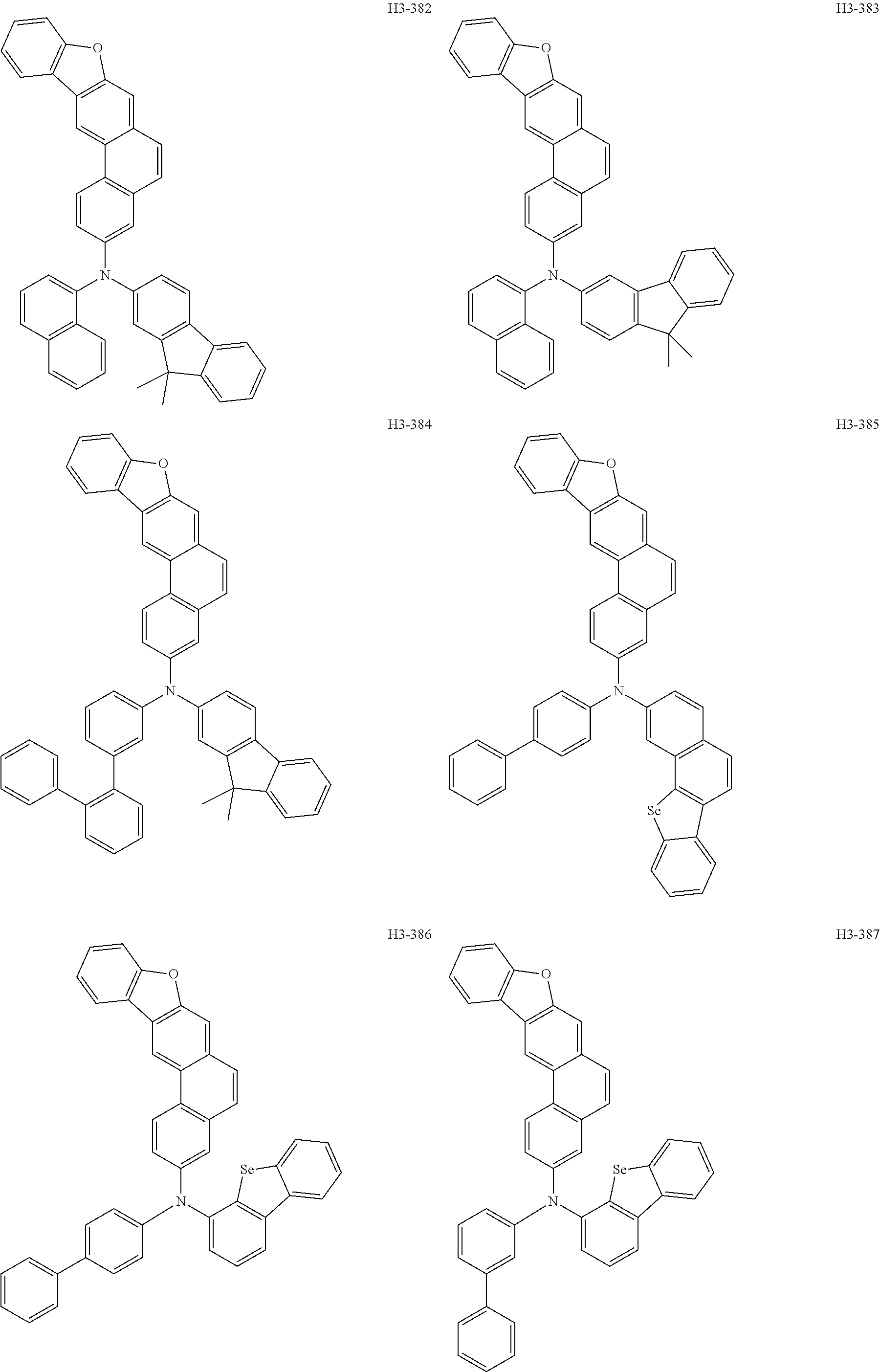 [SMILES: CC1(C)c2ccccc2-c2ccc(N(c3ccc4c(ccc5cc6oc7ccccc7c6cc54)c3)c3cccc4ccccc34)cc21 ; CC1(C)c2ccccc2-c2cc(N(c3ccc4c(ccc5cc6oc7ccccc7c6cc54)c3)c3cccc4ccccc34)ccc21 ; CC1(C)c2ccccc2-c2ccc(N(c3cccc(-c4ccccc4-c4ccccc4)c3)c3ccc4c(ccc5cc6oc7ccccc7c6cc54)c3)cc21 ; c1ccc(-c2ccc(N(c3ccc4c(ccc5cc6oc7ccccc7c6cc54)c3)c3ccc4ccc5c6ccccc6[se]c5c4c3)cc2)cc1 ; c1ccc(-c2ccc(N(c3ccc4c(ccc5cc6oc7ccccc7c6cc54)c3)c3cccc4c3[se]c3ccccc34)cc2)cc1 ; c1ccc(-c2cccc(N(c3ccc4c(ccc5cc6oc7ccccc7c6cc54)c3)c3cccc4c3[se]c3ccccc34)c2)cc1]

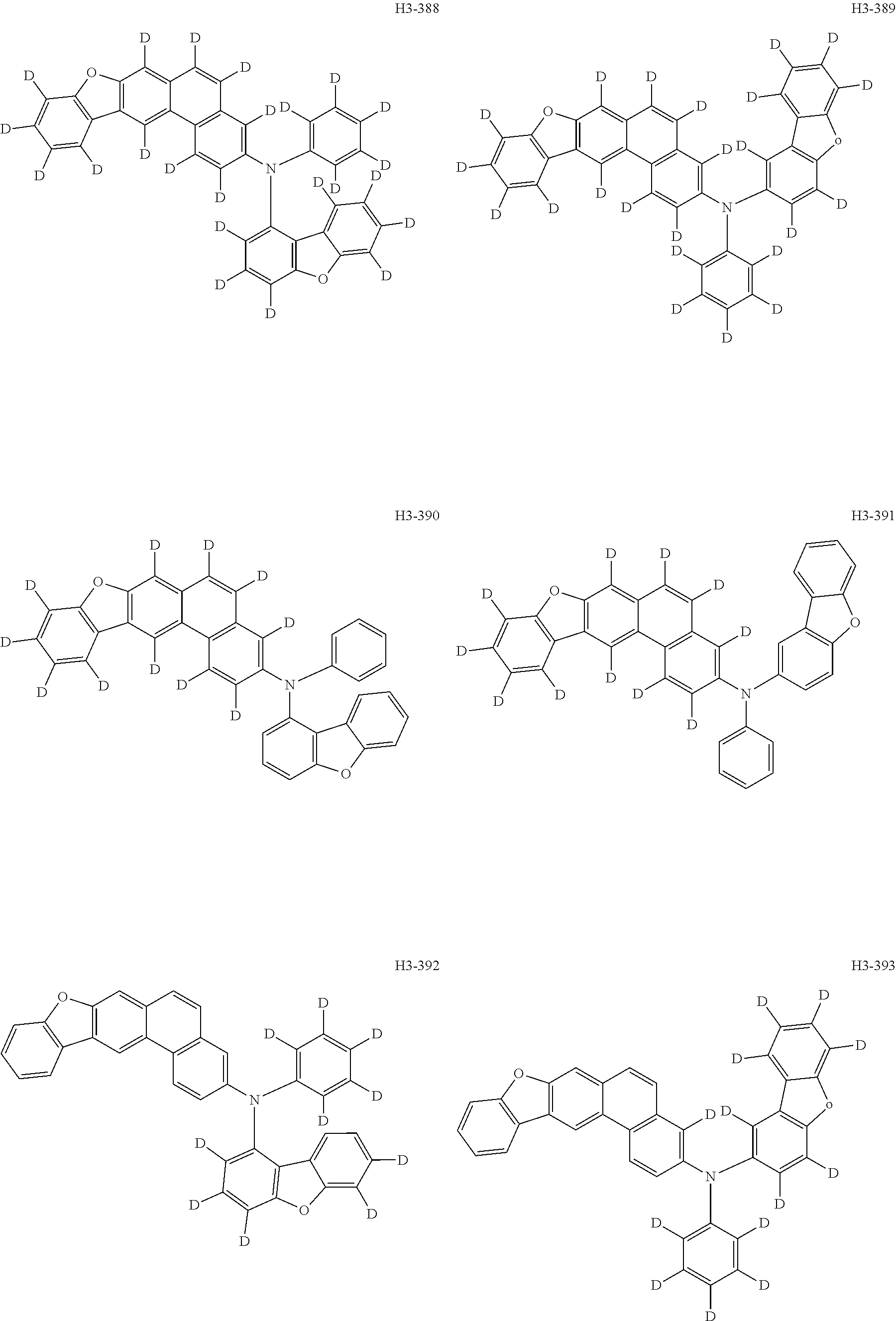

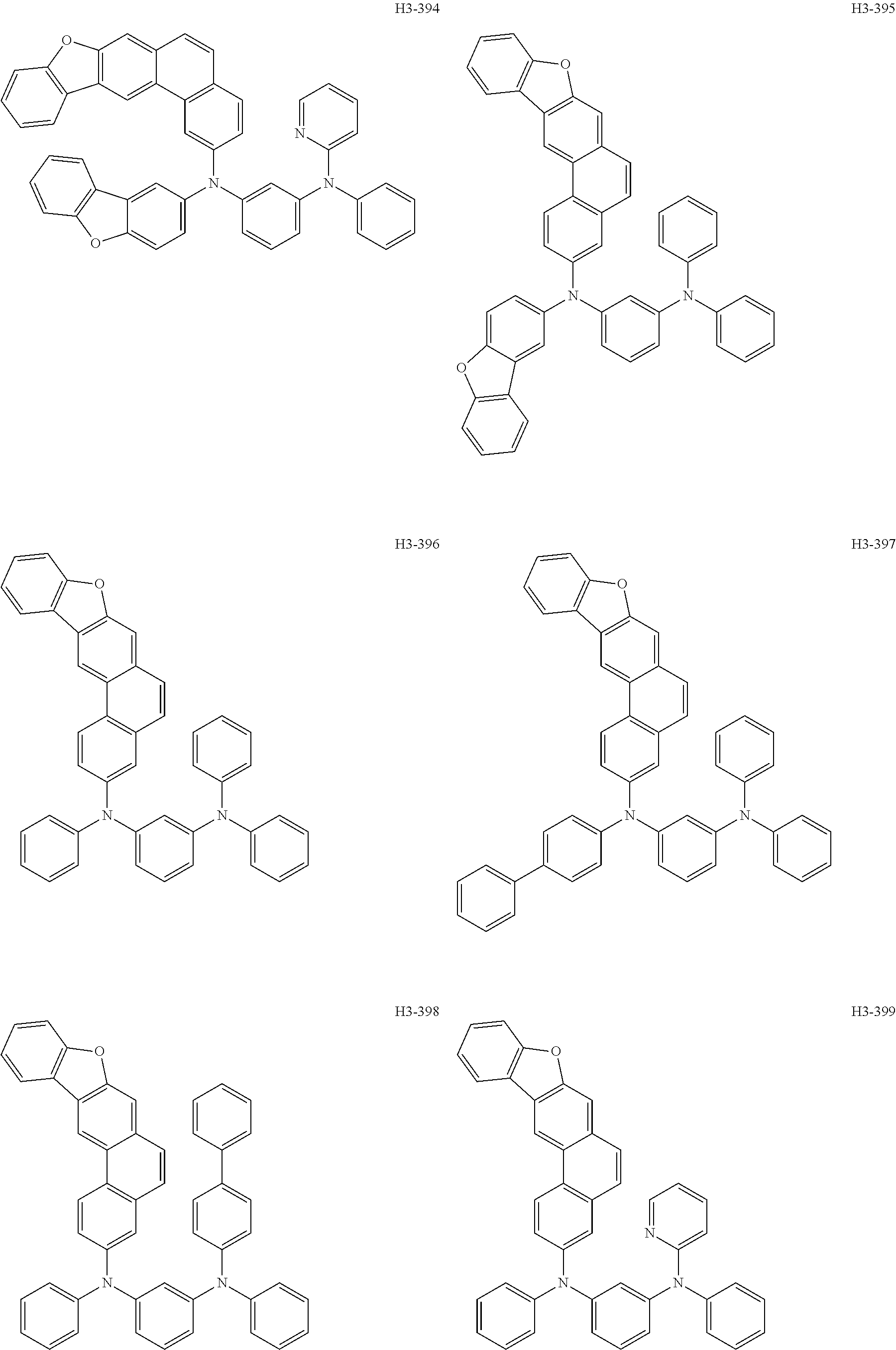

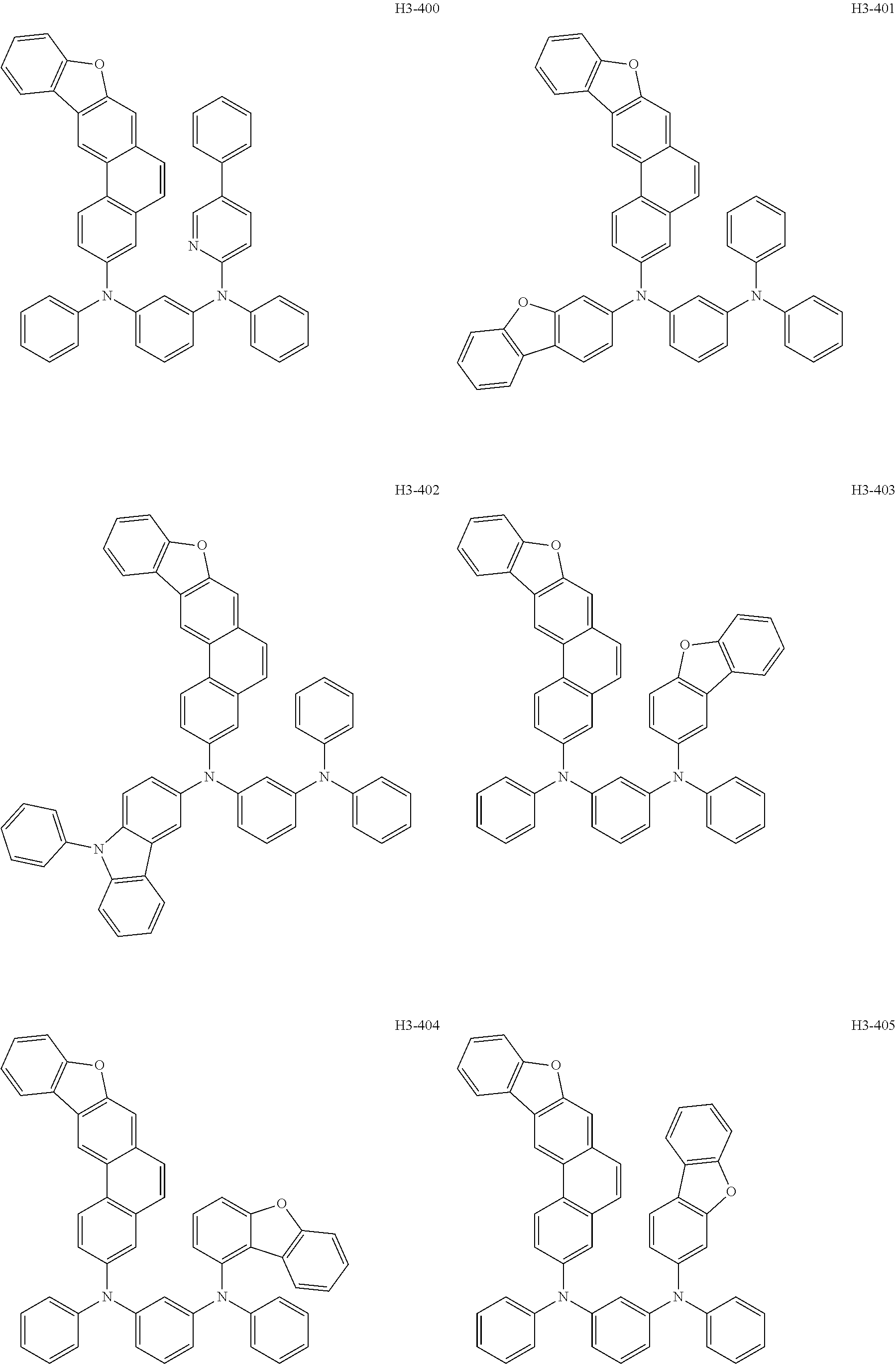

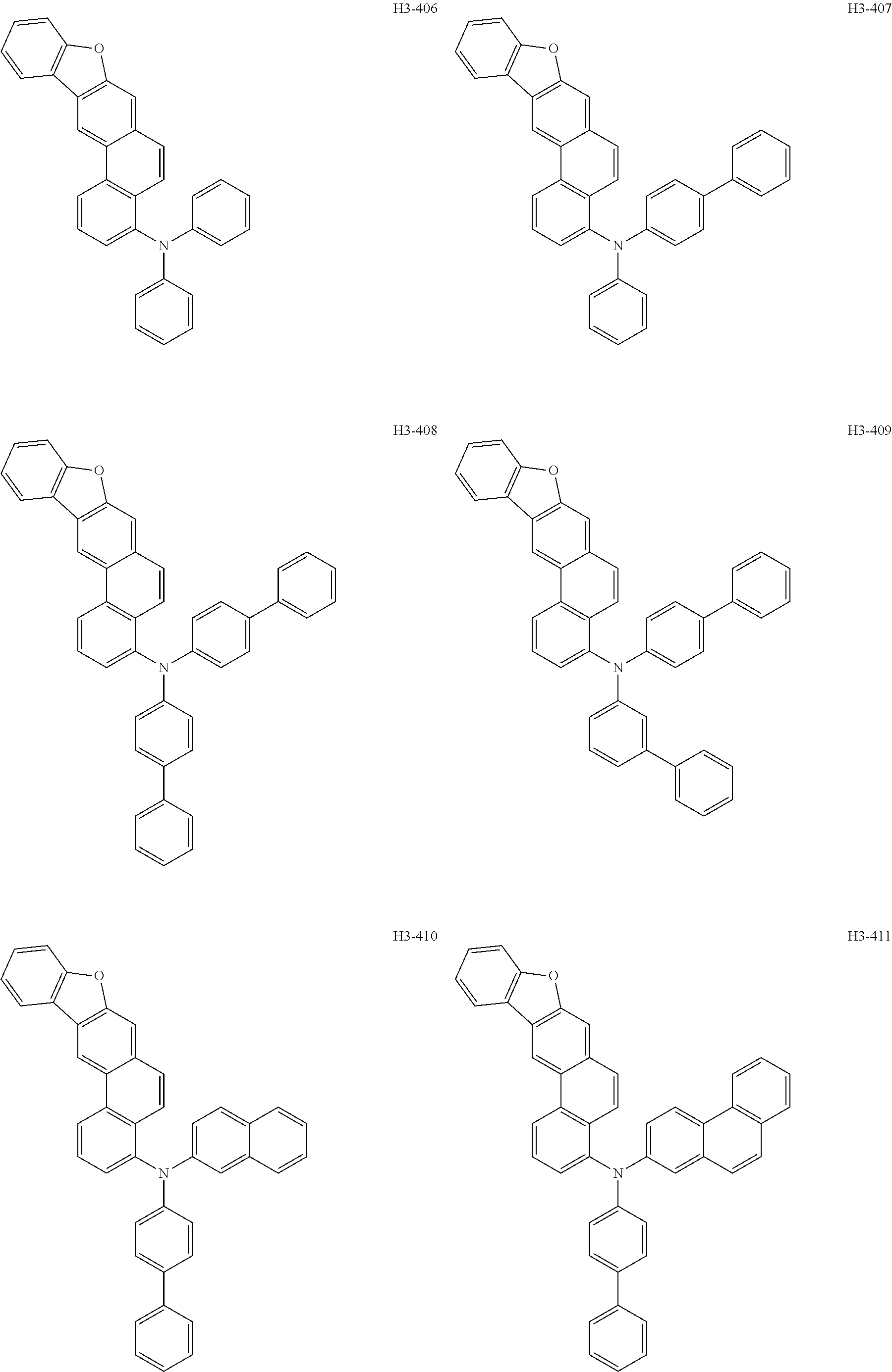 [SMILES: c1ccc(N(c2ccccc2)c2cccc3c2ccc2cc4oc5ccccc5c4cc23)cc1 ; c1ccc(-c2ccc(N(c3ccccc3)c3cccc4c3ccc3cc5oc6ccccc6c5cc34)cc2)cc1 ; c1ccc(-c2ccc(N(c3ccc(-c4ccccc4)cc3)c3cccc4c3ccc3cc5oc6ccccc6c5cc34)cc2)cc1 ; c1ccc(-c2ccc(N(c3cccc(-c4ccccc4)c3)c3cccc4c3ccc3cc5oc6ccccc6c5cc34)cc2)cc1 ; c1ccc(-c2ccc(N(c3ccc4ccccc4c3)c3cccc4c3ccc3cc5oc6ccccc6c5cc34)cc2)cc1 ; c1ccc(-c2ccc(N(c3ccc4c(ccc5ccccc54)c3)c3cccc4c3ccc3cc5oc6ccccc6c5cc34)cc2)cc1]

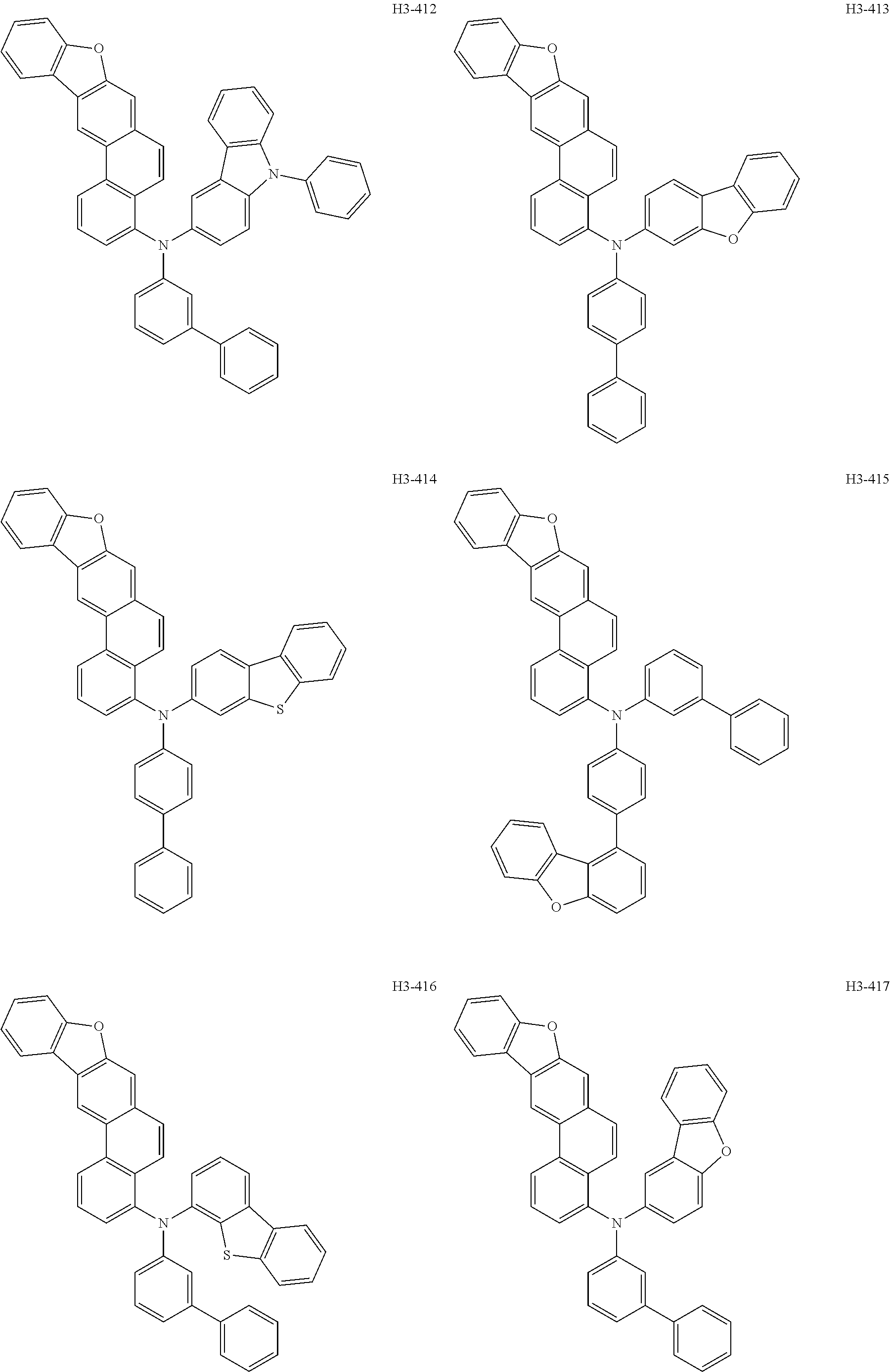 [SMILES: c1ccc(-c2cccc(N(c3ccc4c(c3)c3ccccc3n4-c3ccccc3)c3cccc4c3ccc3cc5oc6ccccc6c5cc34)c2)cc1 ; c1ccc(-c2ccc(N(c3ccc4c(c3)oc3ccccc34)c3cccc4c3ccc3cc5oc6ccccc6c5cc34)cc2)cc1 ; c1ccc(-c2ccc(N(c3ccc4c(c3)sc3ccccc34)c3cccc4c3ccc3cc5oc6ccccc6c5cc34)cc2)cc1 ; c1ccc(-c2cccc(N(c3ccc(-c4cccc5oc6ccccc6c45)cc3)c3cccc4c3ccc3cc5oc6ccccc6c5cc34)c2)cc1 ; c1ccc(-c2cccc(N(c3cccc4c3ccc3cc5oc6ccccc6c5cc34)c3cccc4c3sc3ccccc34)c2)cc1 ; c1ccc(-c2cccc(N(c3ccc4oc5ccccc5c4c3)c3cccc4c3ccc3cc5oc6ccccc6c5cc34)c2)cc1]

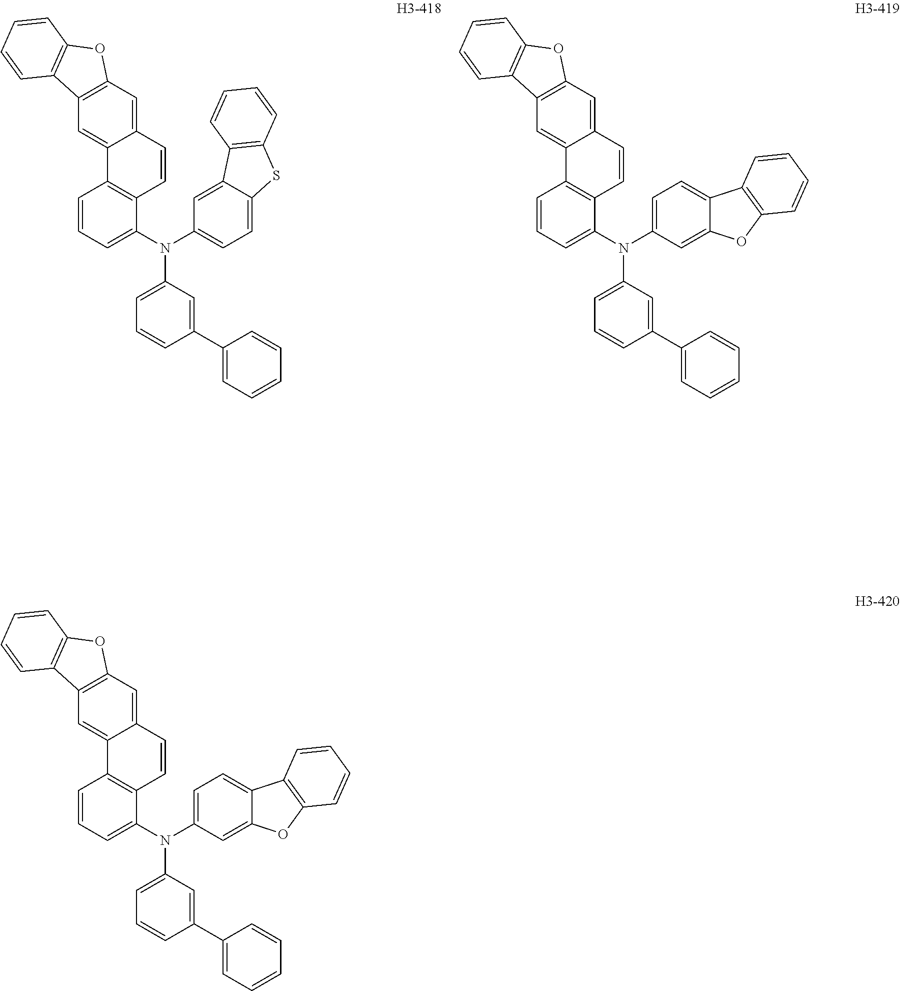 [SMILES: c1ccc(-c2cccc(N(c3ccc4sc5ccccc5c4c3)c3cccc4c3ccc3cc5oc6ccccc6c5cc34)c2)cc1 ; c1ccc(-c2cccc(N(c3ccc4c(c3)oc3ccccc34)c3cccc4c3ccc3cc5oc6ccccc6c5cc34)c2)cc1 ; c1ccc(-c2cccc(N(c3ccc4c(c3)oc3ccccc34)c3cccc4c3ccc3cc5oc6ccccc6c5cc34)c2)cc1]

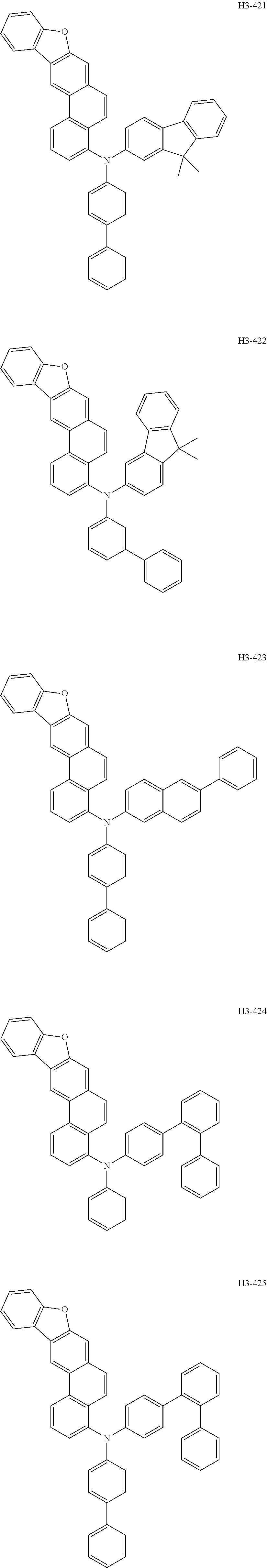 [SMILES: CC1(C)c2ccccc2-c2ccc(N(c3ccc(-c4ccccc4)cc3)c3cccc4c3ccc3cc5oc6ccccc6c5cc34)cc21 ; CC1(C)c2ccccc2-c2cc(N(c3cccc(-c4ccccc4)c3)c3cccc4c3ccc3cc5oc6ccccc6c5cc34)ccc21 ; c1ccc(-c2ccc(N(c3ccc4cc(-c5ccccc5)ccc4c3)c3cccc4c3ccc3cc5oc6ccccc6c5cc34)cc2)cc1 ; c1ccc(-c2ccccc2-c2ccc(N(c3ccccc3)c3cccc4c3ccc3cc5oc6ccccc6c5cc34)cc2)cc1 ; c1ccc(-c2ccc(N(c3ccc(-c4ccccc4-c4ccccc4)cc3)c3cccc4c3ccc3cc5oc6ccccc6c5cc34)cc2)cc1]

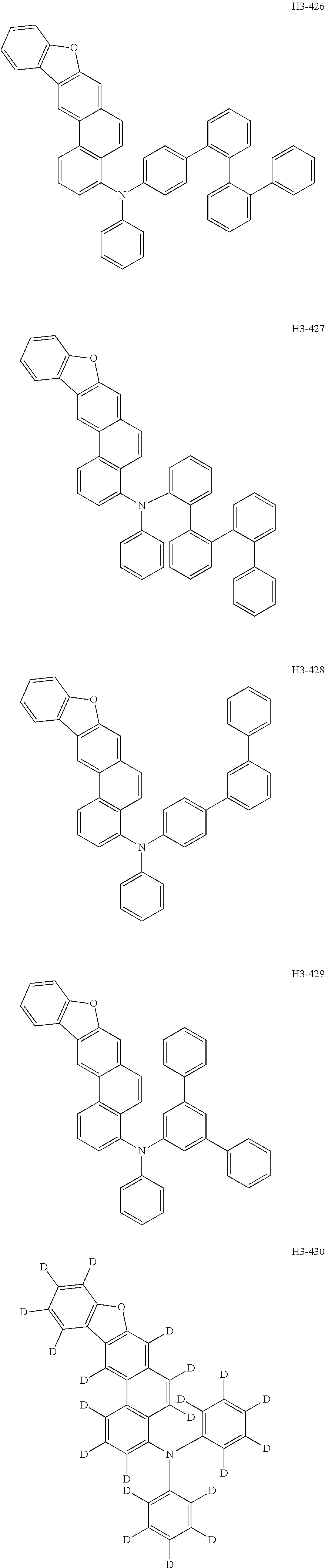 [SMILES: c1ccc(-c2ccccc2-c2ccccc2-c2ccc(N(c3ccccc3)c3cccc4c3ccc3cc5oc6ccccc6c5cc34)cc2)cc1 ; c1ccc(-c2ccccc2-c2ccccc2-c2ccccc2N(c2ccccc2)c2cccc3c2ccc2cc4oc5ccccc5c4cc23)cc1 ; c1ccc(-c2cccc(-c3ccc(N(c4ccccc4)c4cccc5c4ccc4cc6oc7ccccc7c6cc45)cc3)c2)cc1 ; c1ccc(-c2cc(-c3ccccc3)cc(N(c3ccccc3)c3cccc4c3ccc3cc5oc6ccccc6c5cc34)c2)cc1 ; [2H]c1c([2H])c([2H])c(N(c2c([2H])c([2H])c([2H])c([2H])c2[2H])c2c([2H])c([2H])c([2H])c3c2c([2H])c([2H])c2c([2H])c4oc5c([2H])c([2H])c([2H])c([2H])c5c4c([2H])c23)c([2H])c1[2H]]

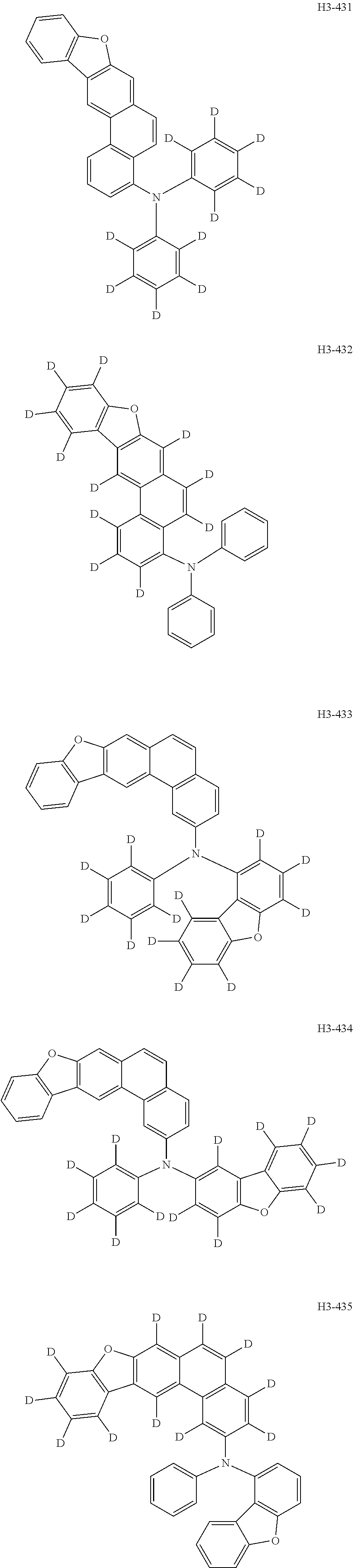

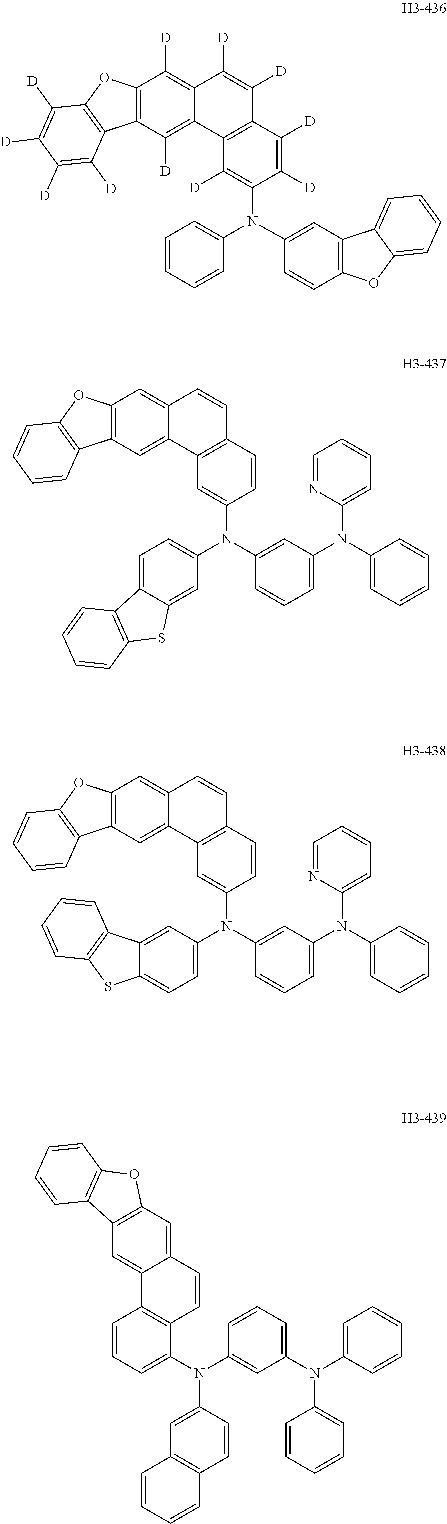 [SMILES: [2H]c1c([2H])c([2H])c2c(oc3c([2H])c4c([2H])c([2H])c5c([2H])c([2H])c(N(c6ccccc6)c6ccc7oc8ccccc8c7c6)c([2H])c5c4c([2H])c32)c1[2H] ; c1ccc(N(c2cccc(N(c3ccc4c(c3)sc3ccccc34)c3ccc4ccc5cc6oc7ccccc7c6cc5c4c3)c2)c2ccccn2)cc1 ; c1ccc(N(c2cccc(N(c3ccc4ccc5cc6oc7ccccc7c6cc5c4c3)c3ccc4sc5ccccc5c4c3)c2)c2ccccn2)cc1 ; c1ccc(N(c2ccccc2)c2cccc(N(c3ccc4ccccc4c3)c3cccc4c3ccc3cc5oc6ccccc6c5cc34)c2)cc1]

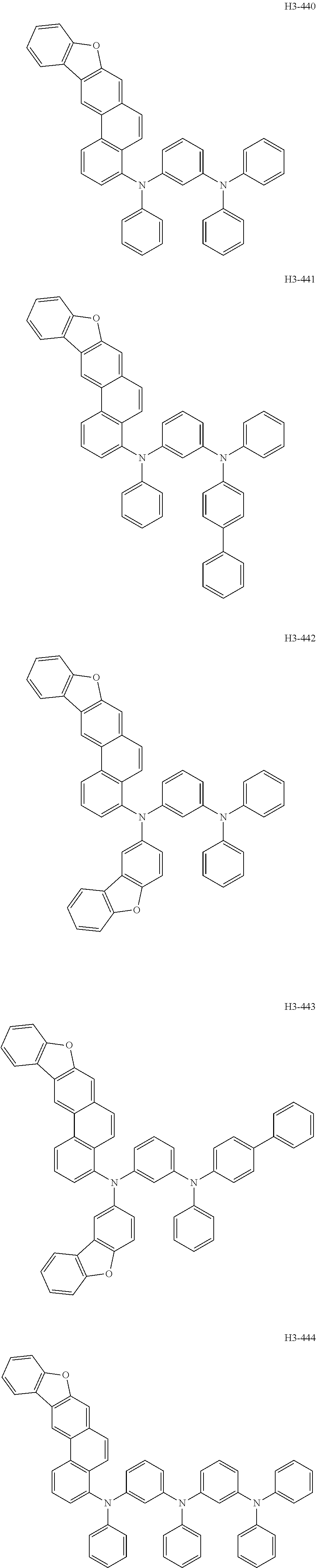 [SMILES: c1ccc(N(c2ccccc2)c2cccc(N(c3ccccc3)c3cccc4c3ccc3cc5oc6ccccc6c5cc34)c2)cc1 ; c1ccc(-c2ccc(N(c3ccccc3)c3cccc(N(c4ccccc4)c4cccc5c4ccc4cc6oc7ccccc7c6cc45)c3)cc2)cc1 ; c1ccc(N(c2ccccc2)c2cccc(N(c3ccc4oc5ccccc5c4c3)c3cccc4c3ccc3cc5oc6ccccc6c5cc34)c2)cc1 ; c1ccc(-c2ccc(N(c3ccccc3)c3cccc(N(c4ccc5oc6ccccc6c5c4)c4cccc5c4ccc4cc6oc7ccccc7c6cc45)c3)cc2)cc1 ; c1ccc(N(c2ccccc2)c2cccc(N(c3ccccc3)c3cccc(N(c4ccccc4)c4cccc5c4ccc4cc6oc7ccccc7c6cc45)c3)c2)cc1]

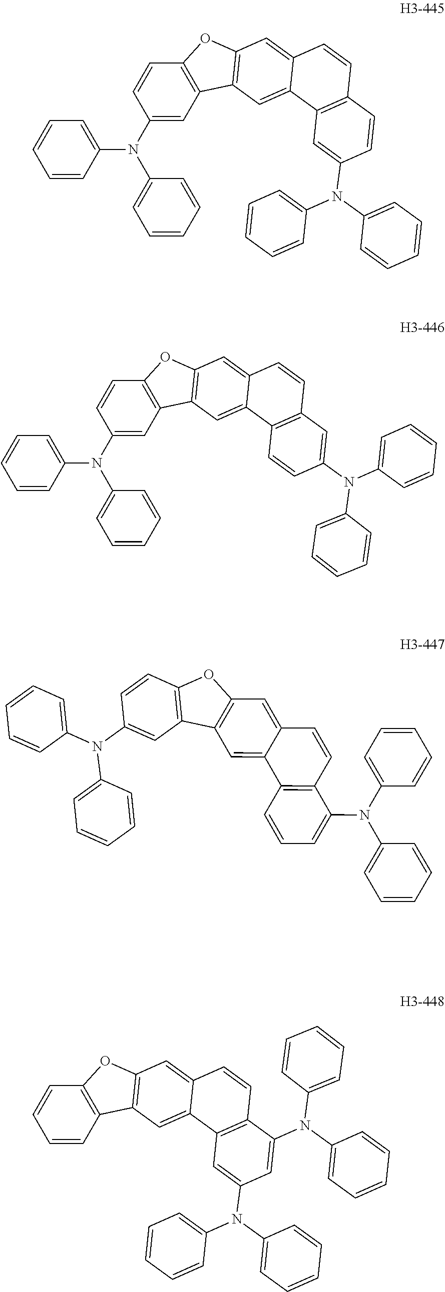 [SMILES: c1ccc(N(c2ccccc2)c2ccc3ccc4cc5oc6ccc(N(c7ccccc7)c7ccccc7)cc6c5cc4c3c2)cc1 ; c1ccc(N(c2ccccc2)c2ccc3c(ccc4cc5oc6ccc(N(c7ccccc7)c7ccccc7)cc6c5cc43)c2)cc1 ; c1ccc(N(c2ccccc2)c2ccc3oc4cc5ccc6c(N(c7ccccc7)c7ccccc7)cccc6c5cc4c3c2)cc1 ; c1ccc(N(c2ccccc2)c2cc(N(c3ccccc3)c3ccccc3)c3ccc4cc5oc6ccccc6c5cc4c3c2)cc1]

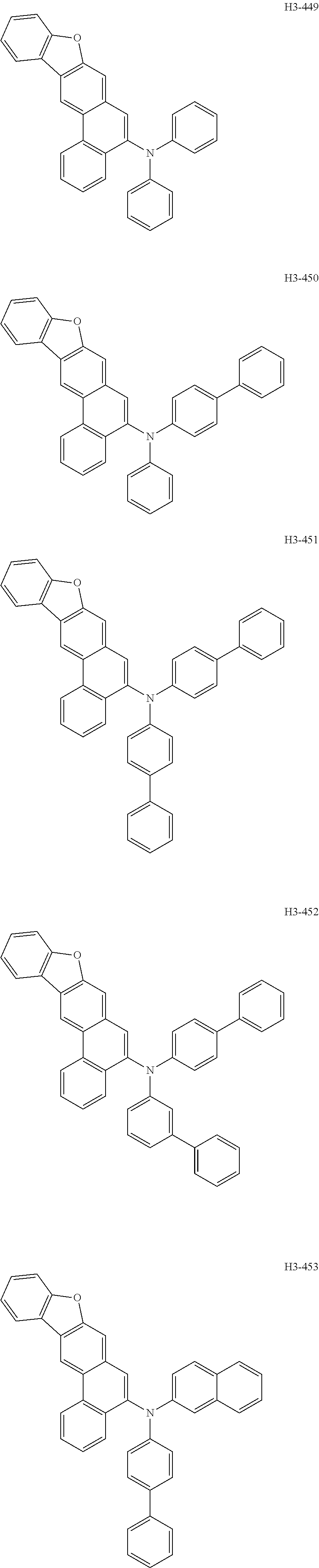 [SMILES: c1ccc(N(c2ccccc2)c2cc3cc4oc5ccccc5c4cc3c3ccccc23)cc1 ; c1ccc(-c2ccc(N(c3ccccc3)c3cc4cc5oc6ccccc6c5cc4c4ccccc34)cc2)cc1 ; c1ccc(-c2ccc(N(c3ccc(-c4ccccc4)cc3)c3cc4cc5oc6ccccc6c5cc4c4ccccc34)cc2)cc1 ; c1ccc(-c2ccc(N(c3cccc(-c4ccccc4)c3)c3cc4cc5oc6ccccc6c5cc4c4ccccc34)cc2)cc1 ; c1ccc(-c2ccc(N(c3ccc4ccccc4c3)c3cc4cc5oc6ccccc6c5cc4c4ccccc34)cc2)cc1]

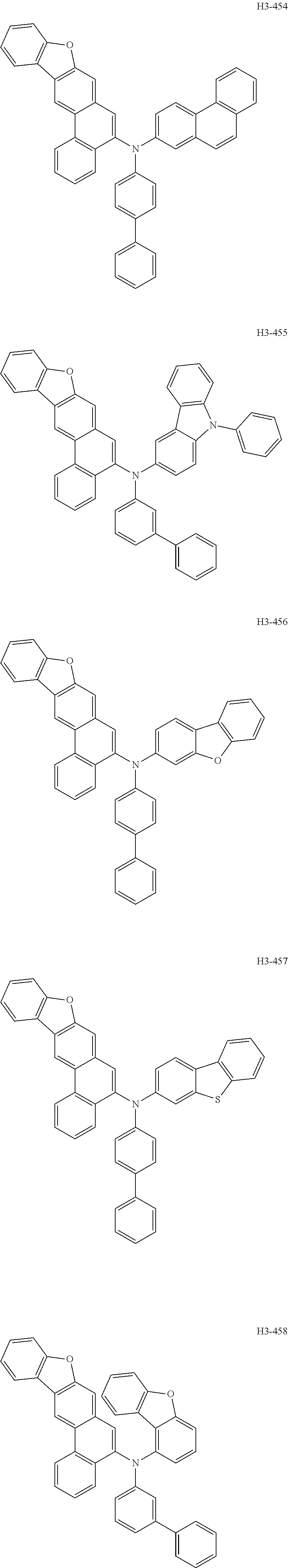 [SMILES: c1ccc(-c2ccc(N(c3ccc4c(ccc5ccccc54)c3)c3cc4cc5oc6ccccc6c5cc4c4ccccc34)cc2)cc1 ; c1ccc(-c2cccc(N(c3ccc4c(c3)c3ccccc3n4-c3ccccc3)c3cc4cc5oc6ccccc6c5cc4c4ccccc34)c2)cc1 ; c1ccc(-c2ccc(N(c3ccc4c(c3)oc3ccccc34)c3cc4cc5oc6ccccc6c5cc4c4ccccc34)cc2)cc1 ; c1ccc(-c2ccc(N(c3ccc4c(c3)sc3ccccc34)c3cc4cc5oc6ccccc6c5cc4c4ccccc34)cc2)cc1 ; c1ccc(-c2cccc(N(c3cc4cc5oc6ccccc6c5cc4c4ccccc34)c3cccc4oc5ccccc5c34)c2)cc1]

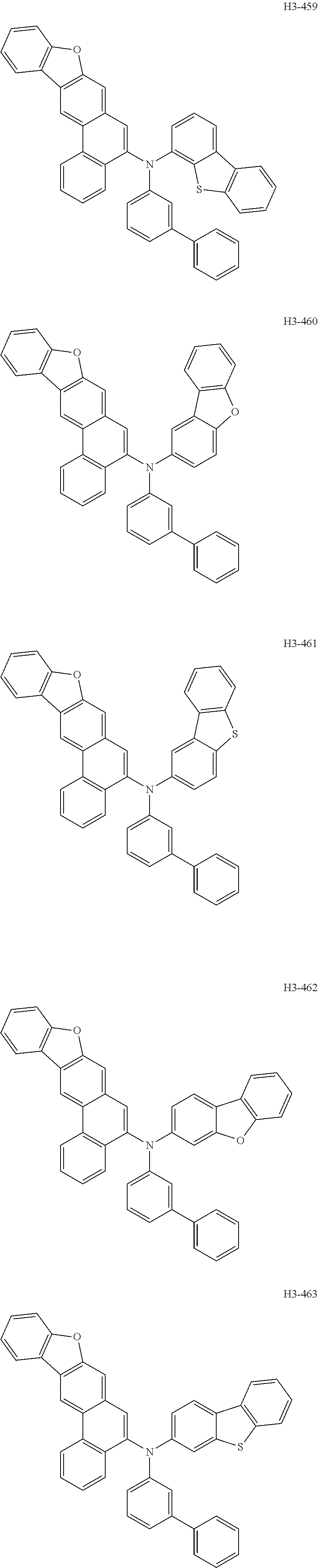 [SMILES: c1ccc(-c2cccc(N(c3cc4cc5oc6ccccc6c5cc4c4ccccc34)c3cccc4c3sc3ccccc34)c2)cc1 ; c1ccc(-c2cccc(N(c3ccc4oc5ccccc5c4c3)c3cc4cc5oc6ccccc6c5cc4c4ccccc34)c2)cc1 ; c1ccc(-c2cccc(N(c3ccc4sc5ccccc5c4c3)c3cc4cc5oc6ccccc6c5cc4c4ccccc34)c2)cc1 ; c1ccc(-c2cccc(N(c3ccc4c(c3)oc3ccccc34)c3cc4cc5oc6ccccc6c5cc4c4ccccc34)c2)cc1 ; c1ccc(-c2cccc(N(c3ccc4c(c3)sc3ccccc34)c3cc4cc5oc6ccccc6c5cc4c4ccccc34)c2)cc1]

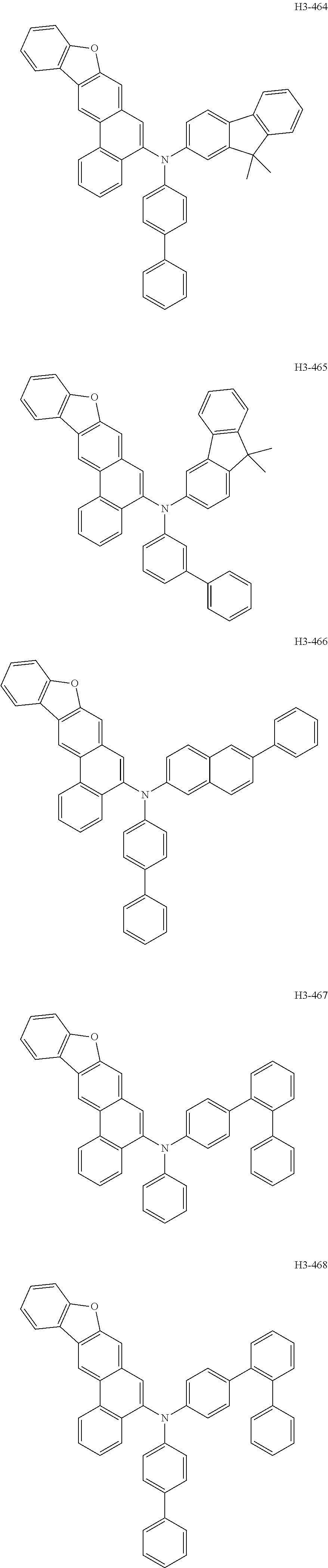 [SMILES: CC1(C)c2ccccc2-c2ccc(N(c3ccc(-c4ccccc4)cc3)c3cc4cc5oc6ccccc6c5cc4c4ccccc34)cc21 ; CC1(C)c2ccccc2-c2cc(N(c3cccc(-c4ccccc4)c3)c3cc4cc5oc6ccccc6c5cc4c4ccccc34)ccc21 ; c1ccc(-c2ccc(N(c3ccc4cc(-c5ccccc5)ccc4c3)c3cc4cc5oc6ccccc6c5cc4c4ccccc34)cc2)cc1 ; c1ccc(-c2ccccc2-c2ccc(N(c3ccccc3)c3cc4cc5oc6ccccc6c5cc4c4ccccc34)cc2)cc1 ; c1ccc(-c2ccc(N(c3ccc(-c4ccccc4-c4ccccc4)cc3)c3cc4cc5oc6ccccc6c5cc4c4ccccc34)cc2)cc1]

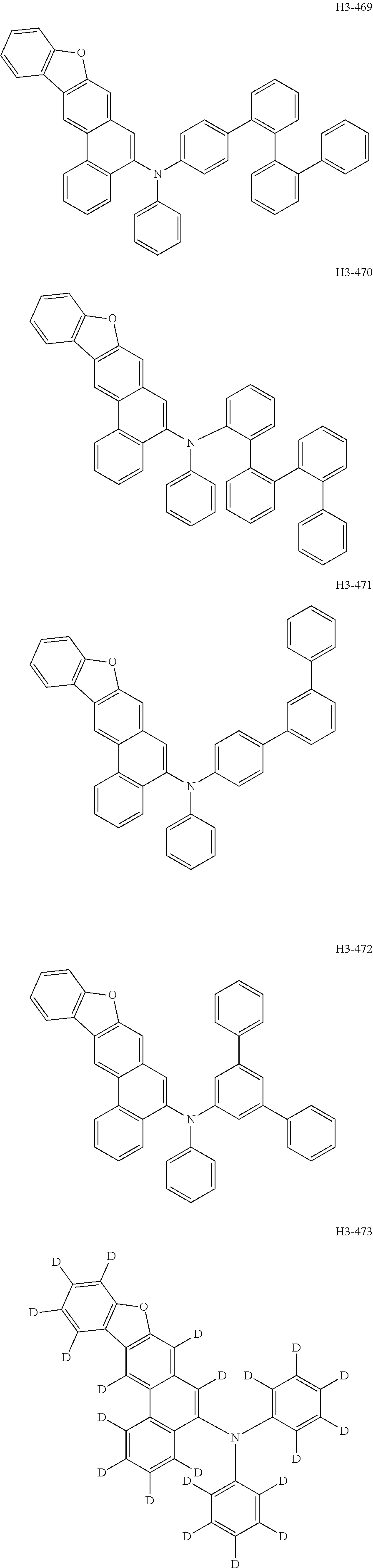 [SMILES: c1ccc(-c2ccccc2-c2ccccc2-c2ccc(N(c3ccccc3)c3cc4cc5oc6ccccc6c5cc4c4ccccc34)cc2)cc1 ; c1ccc(-c2ccccc2-c2ccccc2-c2ccccc2N(c2ccccc2)c2cc3cc4oc5ccccc5c4cc3c3ccccc23)cc1 ; c1ccc(-c2cccc(-c3ccc(N(c4ccccc4)c4cc5cc6oc7ccccc7c6cc5c5ccccc45)cc3)c2)cc1 ; c1ccc(-c2cc(-c3ccccc3)cc(N(c3ccccc3)c3cc4cc5oc6ccccc6c5cc4c4ccccc34)c2)cc1 ; [2H]c1c([2H])c([2H])c(N(c2c([2H])c([2H])c([2H])c([2H])c2[2H])c2c([2H])c3c([2H])c4oc5c([2H])c([2H])c([2H])c([2H])c5c4c([2H])c3c3c([2H])c([2H])c([2H])c([2H])c23)c([2H])c1[2H]]

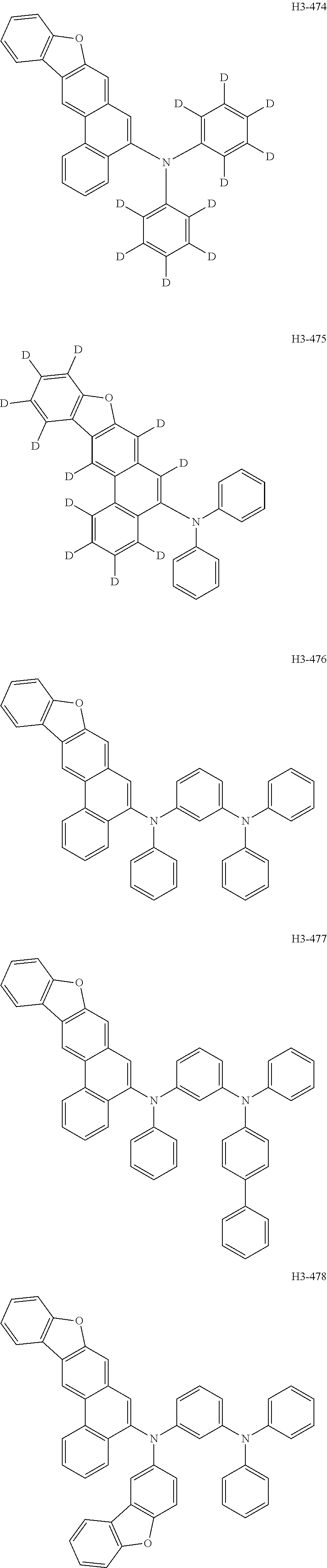 [SMILES: [2H]c1c([2H])c([2H])c(N(c2c([2H])c([2H])c([2H])c([2H])c2[2H])c2cc3cc4oc5ccccc5c4cc3c3ccccc23)c([2H])c1[2H] ; [2H]c1c([2H])c([2H])c2c(oc3c([2H])c4c([2H])c(N(c5ccccc5)c5ccccc5)c5c([2H])c([2H])c([2H])c([2H])c5c4c([2H])c32)c1[2H] ; c1ccc(N(c2ccccc2)c2cccc(N(c3ccccc3)c3cc4cc5oc6ccccc6c5cc4c4ccccc34)c2)cc1 ; c1ccc(-c2ccc(N(c3ccccc3)c3cccc(N(c4ccccc4)c4cc5cc6oc7ccccc7c6cc5c5ccccc45)c3)cc2)cc1 ; c1ccc(N(c2ccccc2)c2cccc(N(c3ccc4oc5ccccc5c4c3)c3cc4cc5oc6ccccc6c5cc4c4ccccc34)c2)cc1]

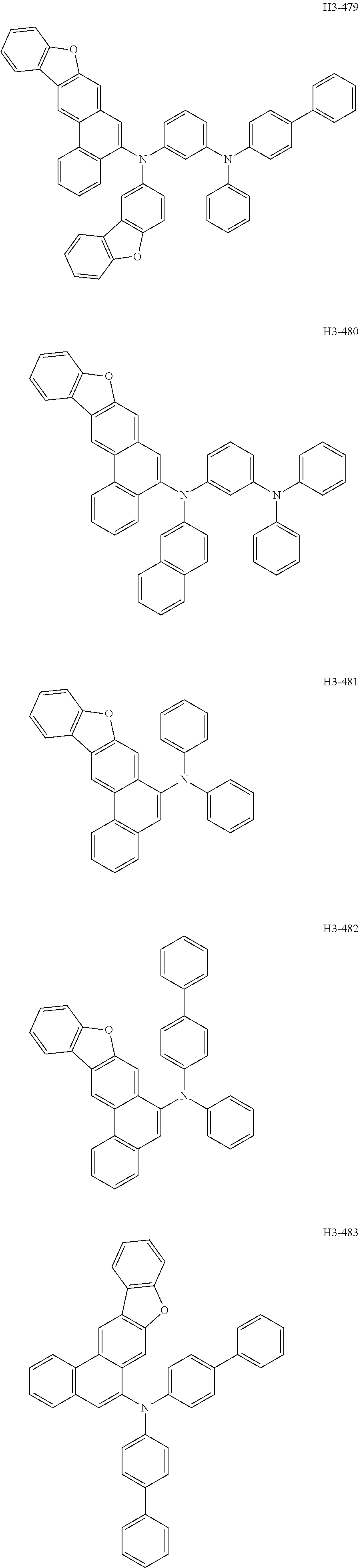 [SMILES: c1ccc(-c2ccc(N(c3ccccc3)c3cccc(N(c4ccc5oc6ccccc6c5c4)c4cc5cc6oc7ccccc7c6cc5c5ccccc45)c3)cc2)cc1 ; c1ccc(N(c2ccccc2)c2cccc(N(c3ccc4ccccc4c3)c3cc4cc5oc6ccccc6c5cc4c4ccccc34)c2)cc1 ; c1ccc(N(c2ccccc2)c2cc3ccccc3c3cc4c(cc23)oc2ccccc24)cc1 ; c1ccc(-c2ccc(N(c3ccccc3)c3cc4ccccc4c4cc5c(cc34)oc3ccccc35)cc2)cc1 ; c1ccc(-c2ccc(N(c3ccc(-c4ccccc4)cc3)c3cc4ccccc4c4cc5c(cc34)oc3ccccc35)cc2)cc1]

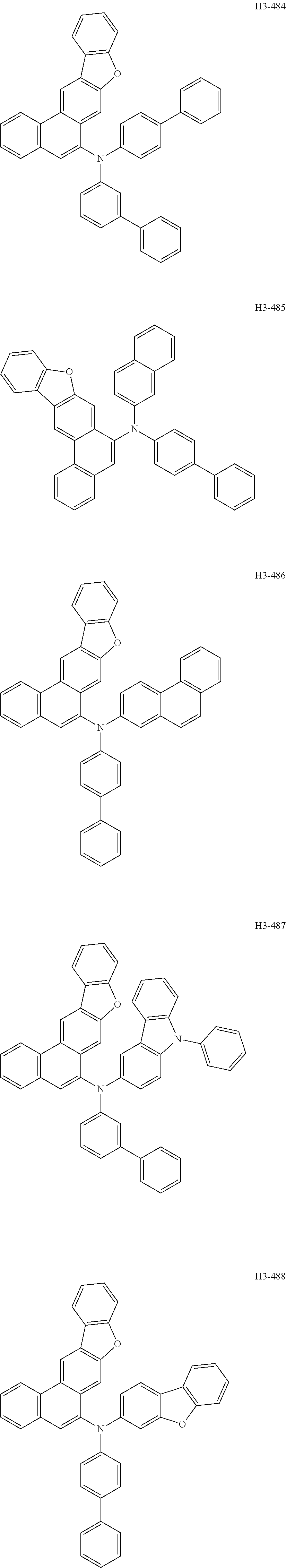 [SMILES: c1ccc(-c2ccc(N(c3cccc(-c4ccccc4)c3)c3cc4ccccc4c4cc5c(cc34)oc3ccccc35)cc2)cc1 ; c1ccc(-c2ccc(N(c3ccc4ccccc4c3)c3cc4ccccc4c4cc5c(cc34)oc3ccccc35)cc2)cc1 ; c1ccc(-c2ccc(N(c3ccc4c(ccc5ccccc54)c3)c3cc4ccccc4c4cc5c(cc34)oc3ccccc35)cc2)cc1 ; c1ccc(-c2cccc(N(c3ccc4c(c3)c3ccccc3n4-c3ccccc3)c3cc4ccccc4c4cc5c(cc34)oc3ccccc35)c2)cc1 ; c1ccc(-c2ccc(N(c3ccc4c(c3)oc3ccccc34)c3cc4ccccc4c4cc5c(cc34)oc3ccccc35)cc2)cc1]

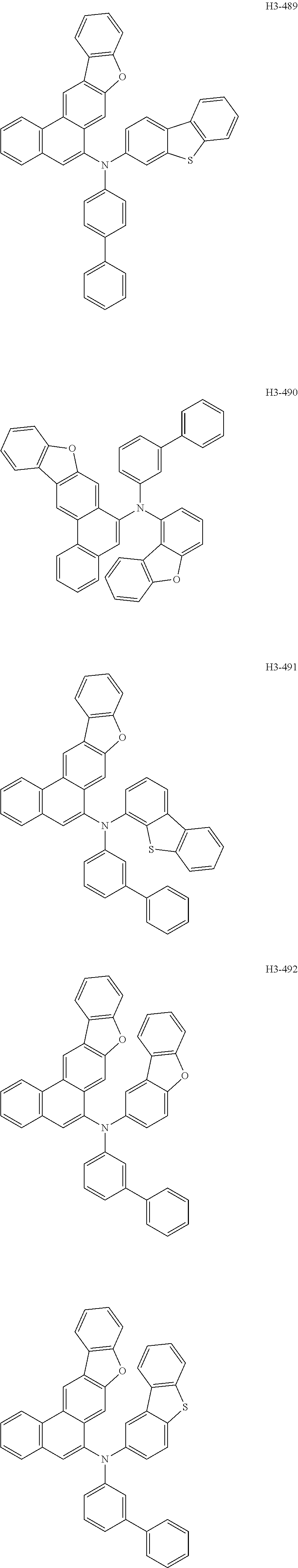 [SMILES: c1ccc(-c2ccc(N(c3ccc4c(c3)sc3ccccc34)c3cc4ccccc4c4cc5c(cc34)oc3ccccc35)cc2)cc1 ; c1ccc(-c2cccc(N(c3cc4ccccc4c4cc5c(cc34)oc3ccccc35)c3cccc4oc5ccccc5c34)c2)cc1 ; c1ccc(-c2cccc(N(c3cc4ccccc4c4cc5c(cc34)oc3ccccc35)c3cccc4c3sc3ccccc34)c2)cc1 ; c1ccc(-c2cccc(N(c3ccc4oc5ccccc5c4c3)c3cc4ccccc4c4cc5c(cc34)oc3ccccc35)c2)cc1 ; c1ccc(-c2cccc(N(c3ccc4sc5ccccc5c4c3)c3cc4ccccc4c4cc5c(cc34)oc3ccccc35)c2)cc1]

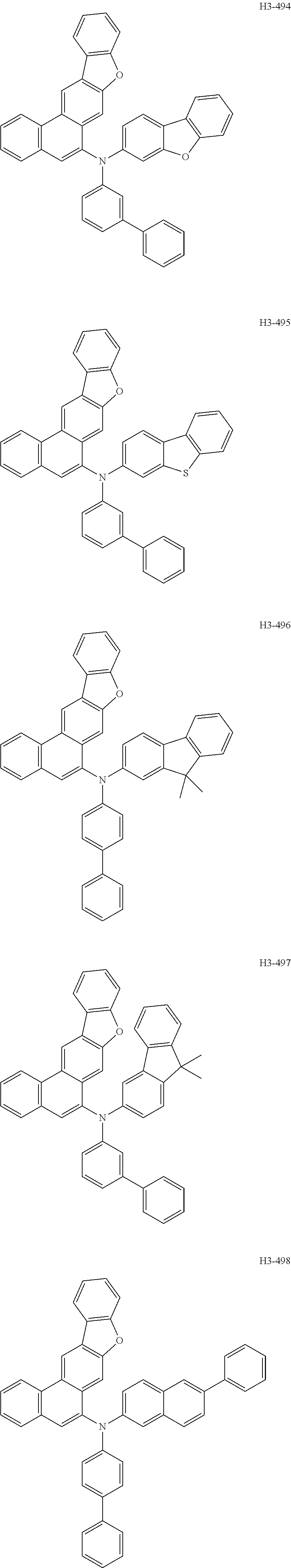 [SMILES: c1ccc(-c2cccc(N(c3ccc4c(c3)oc3ccccc34)c3cc4ccccc4c4cc5c(cc34)oc3ccccc35)c2)cc1 ; c1ccc(-c2cccc(N(c3ccc4c(c3)sc3ccccc34)c3cc4ccccc4c4cc5c(cc34)oc3ccccc35)c2)cc1 ; CC1(C)c2ccccc2-c2ccc(N(c3ccc(-c4ccccc4)cc3)c3cc4ccccc4c4cc5c(cc34)oc3ccccc35)cc21 ; CC1(C)c2ccccc2-c2cc(N(c3cccc(-c4ccccc4)c3)c3cc4ccccc4c4cc5c(cc34)oc3ccccc35)ccc21 ; c1ccc(-c2ccc(N(c3ccc4cc(-c5ccccc5)ccc4c3)c3cc4ccccc4c4cc5c(cc34)oc3ccccc35)cc2)cc1]

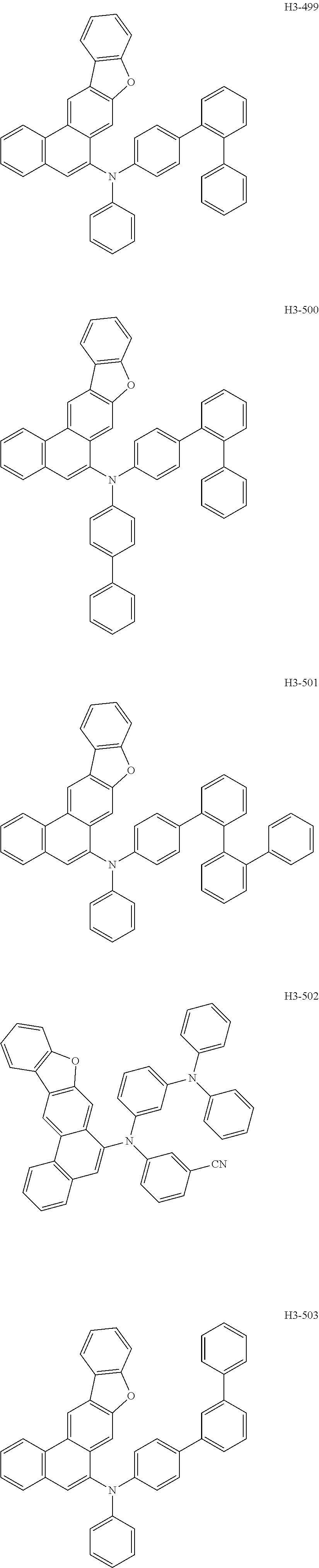 [SMILES: c1ccc(-c2ccccc2-c2ccc(N(c3ccccc3)c3cc4ccccc4c4cc5c(cc34)oc3ccccc35)cc2)cc1 ; c1ccc(-c2ccc(N(c3ccc(-c4ccccc4-c4ccccc4)cc3)c3cc4ccccc4c4cc5c(cc34)oc3ccccc35)cc2)cc1 ; c1ccc(-c2ccccc2-c2ccccc2-c2ccc(N(c3ccccc3)c3cc4ccccc4c4cc5c(cc34)oc3ccccc35)cc2)cc1 ; N#Cc1cccc(N(c2cccc(N(c3ccccc3)c3ccccc3)c2)c2cc3ccccc3c3cc4c(cc23)oc2ccccc24)c1 ; c1ccc(-c2cccc(-c3ccc(N(c4ccccc4)c4cc5ccccc5c5cc6c(cc45)oc4ccccc46)cc3)c2)cc1]

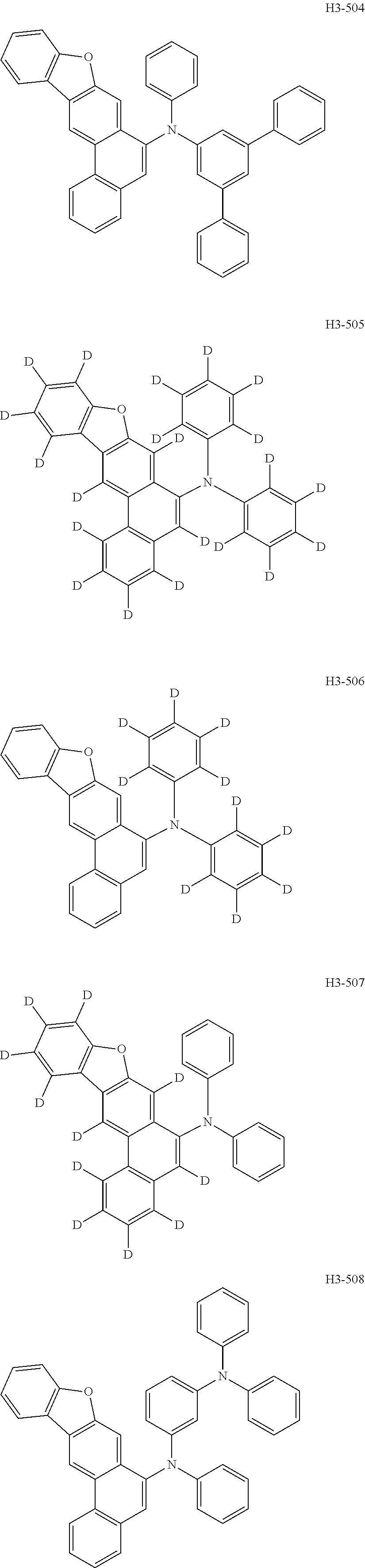

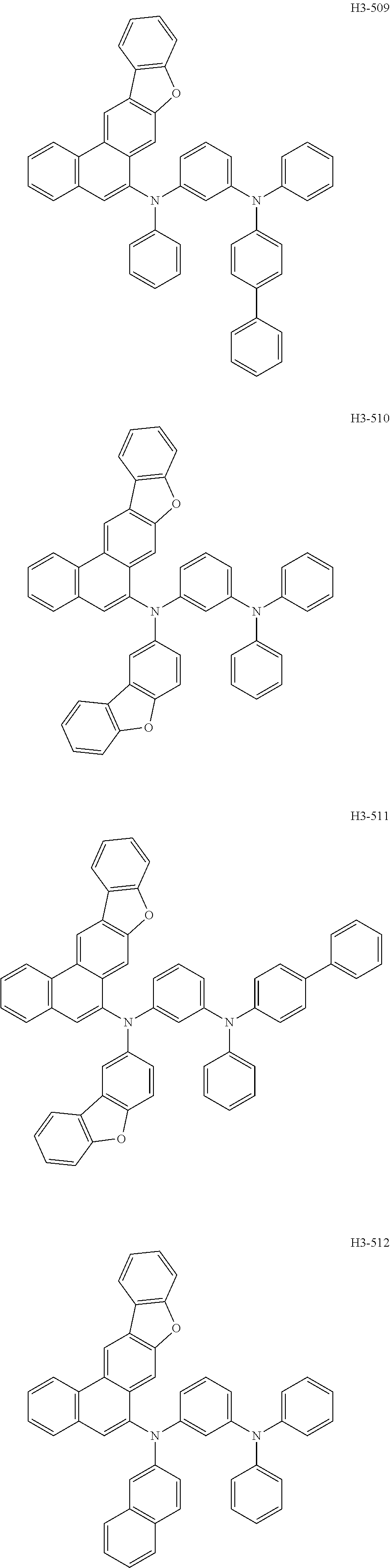 [SMILES: c1ccc(-c2ccc(N(c3ccccc3)c3cccc(N(c4ccccc4)c4cc5ccccc5c5cc6c(cc45)oc4ccccc46)c3)cc2)cc1 ; c1ccc(N(c2ccccc2)c2cccc(N(c3ccc4oc5ccccc5c4c3)c3cc4ccccc4c4cc5c(cc34)oc3ccccc35)c2)cc1 ; c1ccc(-c2ccc(N(c3ccccc3)c3cccc(N(c4ccc5oc6ccccc6c5c4)c4cc5ccccc5c5cc6c(cc45)oc4ccccc46)c3)cc2)cc1 ; c1ccc(N(c2ccccc2)c2cccc(N(c3ccc4ccccc4c3)c3cc4ccccc4c4cc5c(cc34)oc3ccccc35)c2)cc1]

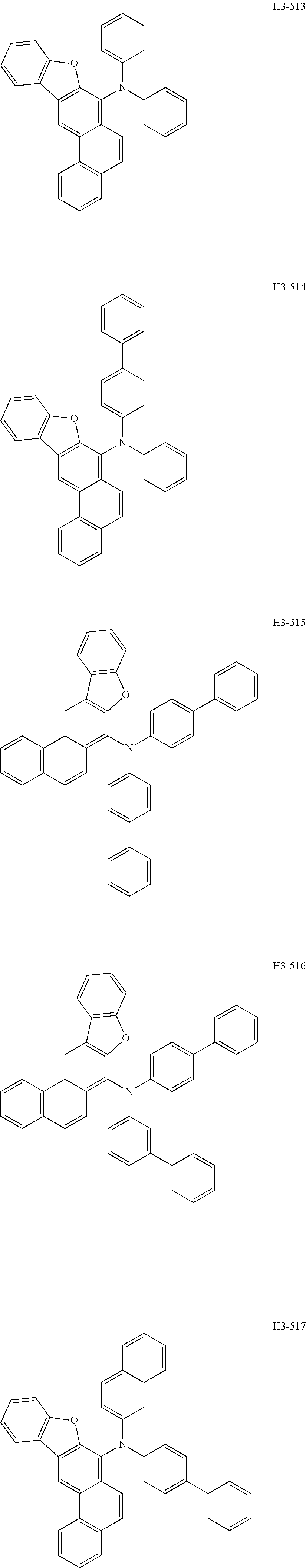 [SMILES: c1ccc(N(c2ccccc2)c2c3ccc4ccccc4c3cc3c2oc2ccccc23)cc1 ; c1ccc(-c2ccc(N(c3ccccc3)c3c4ccc5ccccc5c4cc4c3oc3ccccc34)cc2)cc1 ; c1ccc(-c2ccc(N(c3ccc(-c4ccccc4)cc3)c3c4ccc5ccccc5c4cc4c3oc3ccccc34)cc2)cc1 ; c1ccc(-c2ccc(N(c3cccc(-c4ccccc4)c3)c3c4ccc5ccccc5c4cc4c3oc3ccccc34)cc2)cc1 ; c1ccc(-c2ccc(N(c3ccc4ccccc4c3)c3c4ccc5ccccc5c4cc4c3oc3ccccc34)cc2)cc1]

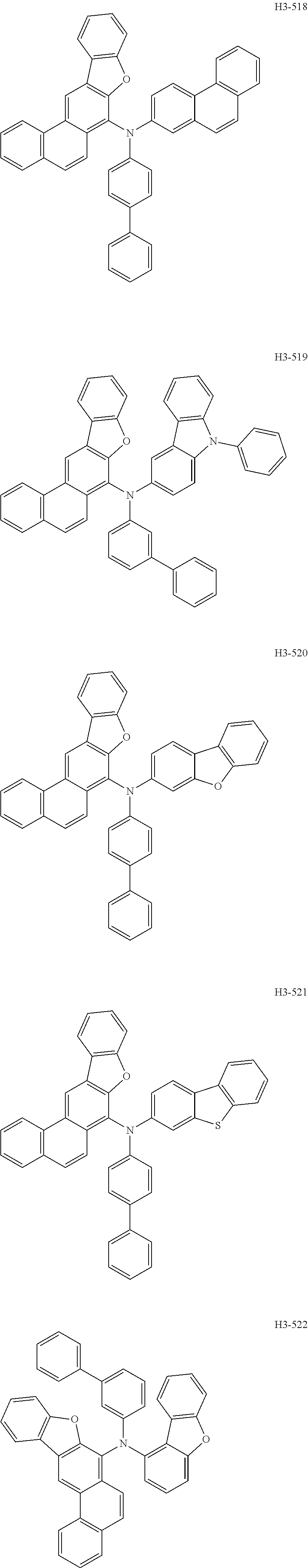 [SMILES: c1ccc(-c2ccc(N(c3ccc4c(ccc5ccccc54)c3)c3c4ccc5ccccc5c4cc4c3oc3ccccc34)cc2)cc1 ; c1ccc(-c2cccc(N(c3ccc4c(c3)c3ccccc3n4-c3ccccc3)c3c4ccc5ccccc5c4cc4c3oc3ccccc34)c2)cc1 ; c1ccc(-c2ccc(N(c3ccc4c(c3)oc3ccccc34)c3c4ccc5ccccc5c4cc4c3oc3ccccc34)cc2)cc1 ; c1ccc(-c2ccc(N(c3ccc4c(c3)sc3ccccc34)c3c4ccc5ccccc5c4cc4c3oc3ccccc34)cc2)cc1 ; c1ccc(-c2cccc(N(c3c4ccc5ccccc5c4cc4c3oc3ccccc34)c3cccc4oc5ccccc5c34)c2)cc1]

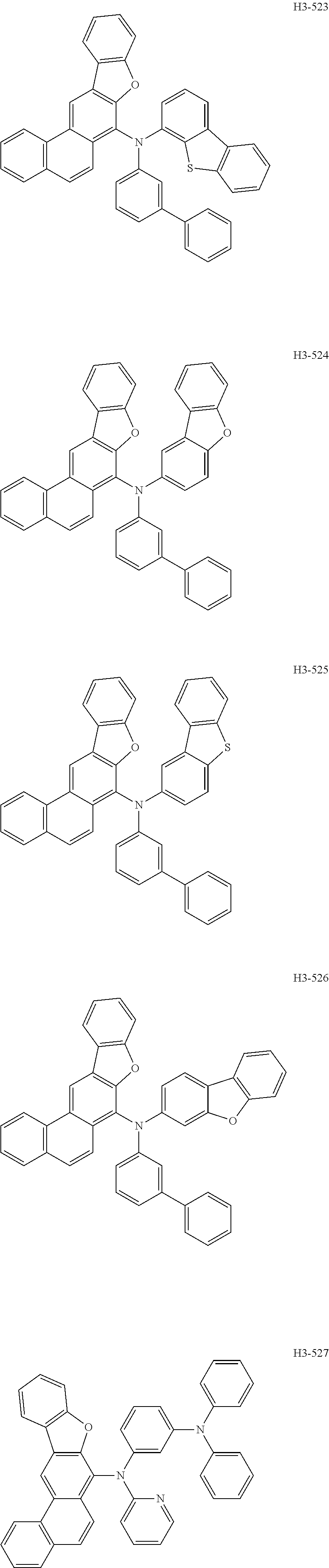 [SMILES: c1ccc(-c2cccc(N(c3c4ccc5ccccc5c4cc4c3oc3ccccc34)c3cccc4c3sc3ccccc34)c2)cc1 ; c1ccc(-c2cccc(N(c3ccc4oc5ccccc5c4c3)c3c4ccc5ccccc5c4cc4c3oc3ccccc34)c2)cc1 ; c1ccc(-c2cccc(N(c3ccc4sc5ccccc5c4c3)c3c4ccc5ccccc5c4cc4c3oc3ccccc34)c2)cc1 ; c1ccc(-c2cccc(N(c3ccc4c(c3)oc3ccccc34)c3c4ccc5ccccc5c4cc4c3oc3ccccc34)c2)cc1 ; c1ccc(N(c2ccccc2)c2cccc(N(c3ccccn3)c3c4ccc5ccccc5c4cc4c3oc3ccccc34)c2)cc1]

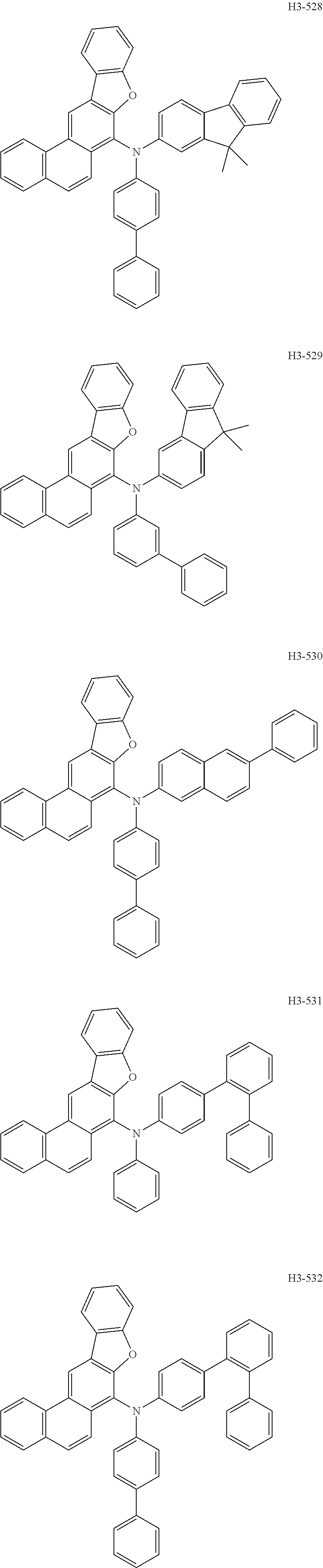 [SMILES: CC1(C)c2ccccc2-c2ccc(N(c3ccc(-c4ccccc4)cc3)c3c4ccc5ccccc5c4cc4c3oc3ccccc34)cc21 ; CC1(C)c2ccccc2-c2cc(N(c3cccc(-c4ccccc4)c3)c3c4ccc5ccccc5c4cc4c3oc3ccccc34)ccc21 ; c1ccc(-c2ccc(N(c3ccc4cc(-c5ccccc5)ccc4c3)c3c4ccc5ccccc5c4cc4c3oc3ccccc34)cc2)cc1 ; c1ccc(-c2ccccc2-c2ccc(N(c3ccccc3)c3c4ccc5ccccc5c4cc4c3oc3ccccc34)cc2)cc1 ; c1ccc(-c2ccc(N(c3ccc(-c4ccccc4-c4ccccc4)cc3)c3c4ccc5ccccc5c4cc4c3oc3ccccc34)cc2)cc1]

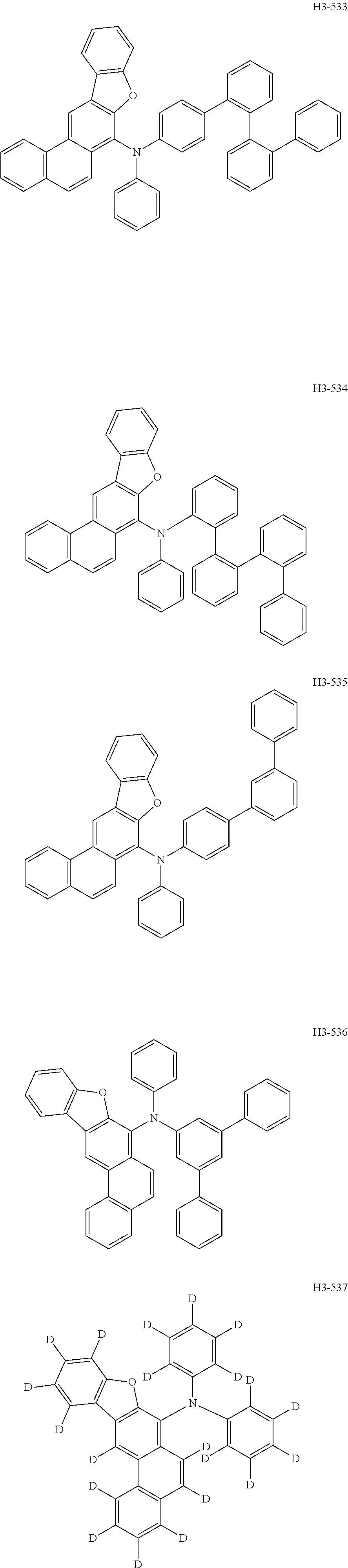 [SMILES: c1ccc(-c2ccccc2-c2ccccc2-c2ccc(N(c3ccccc3)c3c4ccc5ccccc5c4cc4c3oc3ccccc34)cc2)cc1 ; c1ccc(-c2ccccc2-c2ccccc2-c2ccccc2N(c2ccccc2)c2c3ccc4ccccc4c3cc3c2oc2ccccc23)cc1 ; c1ccc(-c2cccc(-c3ccc(N(c4ccccc4)c4c5ccc6ccccc6c5cc5c4oc4ccccc45)cc3)c2)cc1 ; c1ccc(-c2cc(-c3ccccc3)cc(N(c3ccccc3)c3c4ccc5ccccc5c4cc4c3oc3ccccc34)c2)cc1 ; [2H]c1c([2H])c([2H])c(N(c2c([2H])c([2H])c([2H])c([2H])c2[2H])c2c3oc4c([2H])c([2H])c([2H])c([2H])c4c3c([2H])c3c2c([2H])c([2H])c2c([2H])c([2H])c([2H])c([2H])c23)c([2H])c1[2H]]

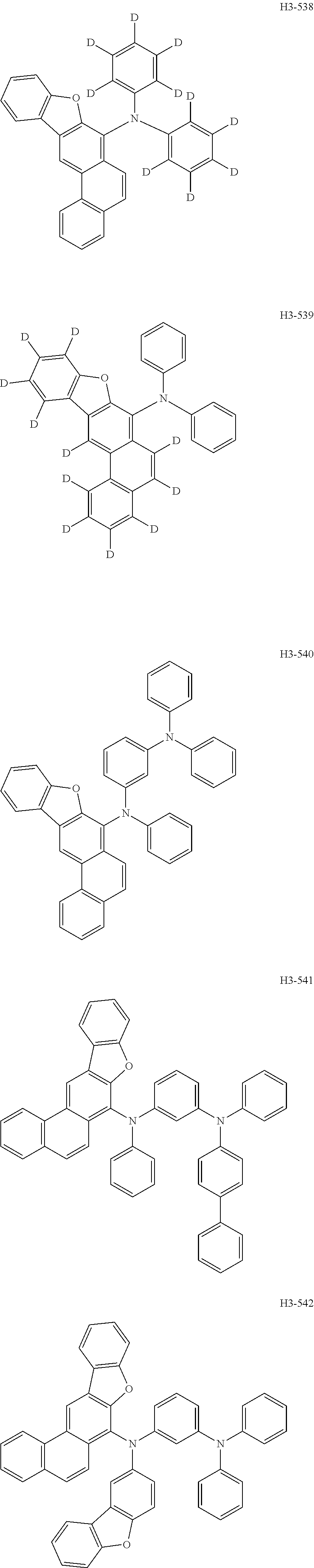 [SMILES: [2H]c1c([2H])c([2H])c(N(c2c([2H])c([2H])c([2H])c([2H])c2[2H])c2c3ccc4ccccc4c3cc3c2oc2ccccc23)c([2H])c1[2H] ; [2H]c1c([2H])c([2H])c2c(oc3c(N(c4ccccc4)c4ccccc4)c4c([2H])c([2H])c5c([2H])c([2H])c([2H])c([2H])c5c4c([2H])c32)c1[2H] ; c1ccc(N(c2ccccc2)c2cccc(N(c3ccccc3)c3c4ccc5ccccc5c4cc4c3oc3ccccc34)c2)cc1 ; c1ccc(-c2ccc(N(c3ccccc3)c3cccc(N(c4ccccc4)c4c5ccc6ccccc6c5cc5c4oc4ccccc45)c3)cc2)cc1 ; c1ccc(N(c2ccccc2)c2cccc(N(c3ccc4oc5ccccc5c4c3)c3c4ccc5ccccc5c4cc4c3oc3ccccc34)c2)cc1]

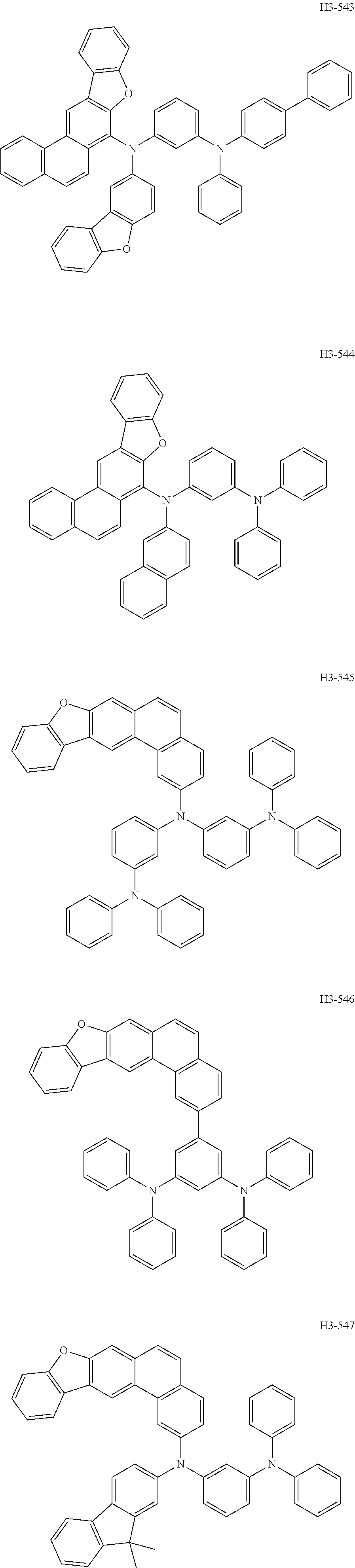 [SMILES: c1ccc(-c2ccc(N(c3ccccc3)c3cccc(N(c4ccc5oc6ccccc6c5c4)c4c5ccc6ccccc6c5cc5c4oc4ccccc45)c3)cc2)cc1 ; c1ccc(N(c2ccccc2)c2cccc(N(c3ccc4ccccc4c3)c3c4ccc5ccccc5c4cc4c3oc3ccccc34)c2)cc1 ; c1ccc(N(c2ccccc2)c2cccc(N(c3cccc(N(c4ccccc4)c4ccccc4)c3)c3ccc4ccc5cc6oc7ccccc7c6cc5c4c3)c2)cc1 ; c1ccc(N(c2ccccc2)c2cc(-c3ccc4ccc5cc6oc7ccccc7c6cc5c4c3)cc(N(c3ccccc3)c3ccccc3)c2)cc1 ; CC1(C)c2ccccc2-c2ccc(N(c3cccc(N(c4ccccc4)c4ccccc4)c3)c3ccc4ccc5cc6oc7ccccc7c6cc5c4c3)cc21]

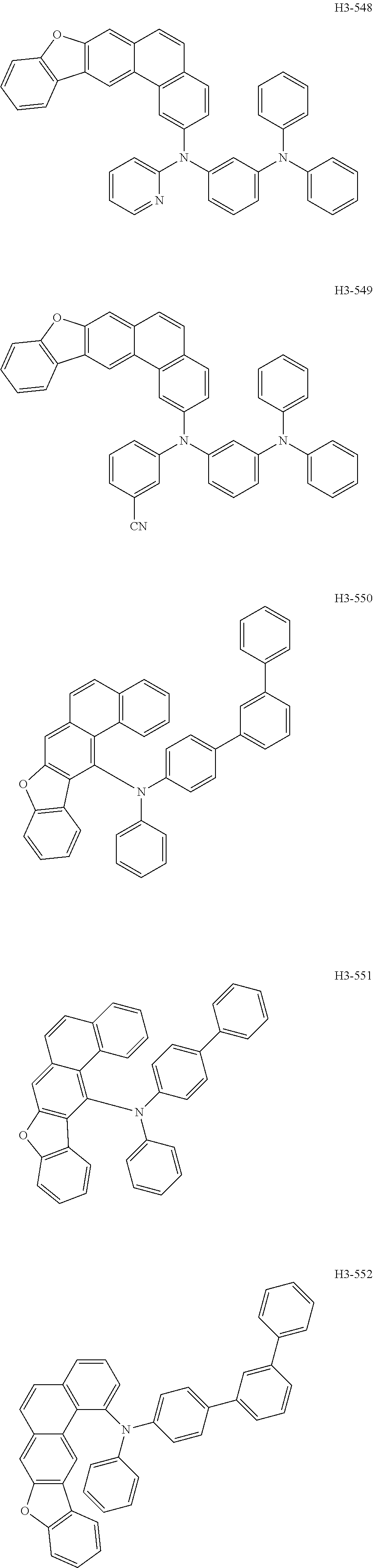 [SMILES: c1ccc(N(c2ccccc2)c2cccc(N(c3ccc4ccc5cc6oc7ccccc7c6cc5c4c3)c3ccccn3)c2)cc1 ; N#Cc1cccc(N(c2cccc(N(c3ccccc3)c3ccccc3)c2)c2ccc3ccc4cc5oc6ccccc6c5cc4c3c2)c1 ; c1ccc(-c2cccc(-c3ccc(N(c4ccccc4)c4c5c(ccc6ccccc65)cc5oc6ccccc6c45)cc3)c2)cc1 ; c1ccc(-c2ccc(N(c3ccccc3)c3c4c(ccc5ccccc54)cc4oc5ccccc5c34)cc2)cc1 ; c1ccc(-c2cccc(-c3ccc(N(c4ccccc4)c4cccc5ccc6cc7oc8ccccc8c7cc6c45)cc3)c2)cc1]

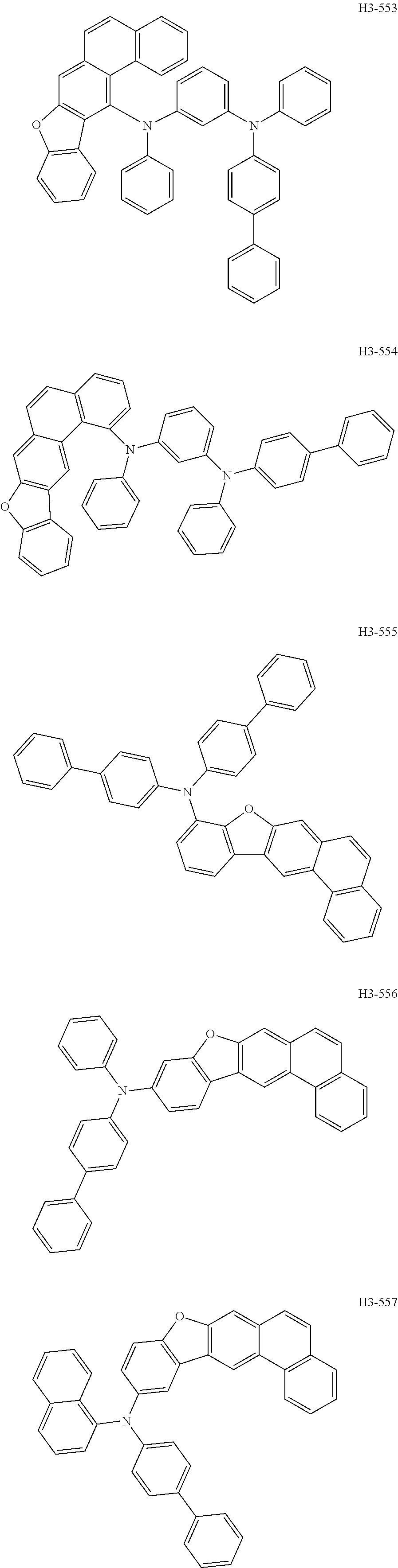 [SMILES: c1ccc(-c2ccc(N(c3ccccc3)c3cccc(N(c4ccccc4)c4c5c(ccc6ccccc65)cc5oc6ccccc6c45)c3)cc2)cc1 ; c1ccc(-c2ccc(N(c3ccccc3)c3cccc(N(c4ccccc4)c4cccc5ccc6cc7oc8ccccc8c7cc6c45)c3)cc2)cc1 ; c1ccc(-c2ccc(N(c3ccc(-c4ccccc4)cc3)c3cccc4c3oc3cc5ccc6ccccc6c5cc34)cc2)cc1 ; c1ccc(-c2ccc(N(c3ccccc3)c3ccc4c(c3)oc3cc5ccc6ccccc6c5cc34)cc2)cc1 ; c1ccc(-c2ccc(N(c3ccc4oc5cc6ccc7ccccc7c6cc5c4c3)c3cccc4ccccc34)cc2)cc1]

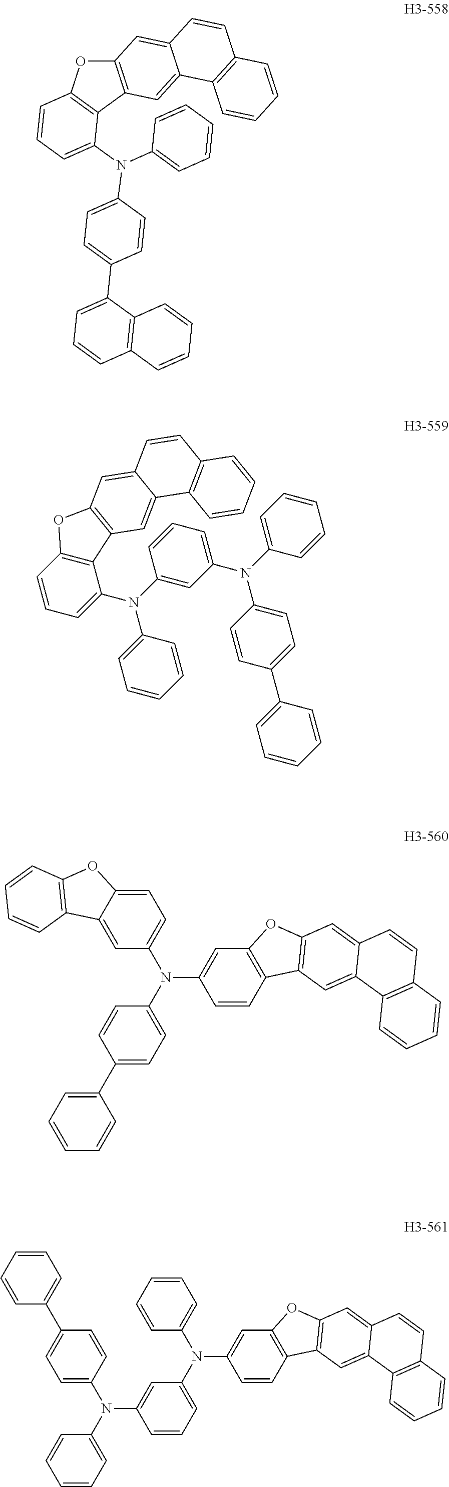 [SMILES: c1ccc(N(c2ccc(-c3cccc4ccccc34)cc2)c2cccc3oc4cc5ccc6ccccc6c5cc4c23)cc1 ; c1ccc(-c2ccc(N(c3ccccc3)c3cccc(N(c4ccccc4)c4cccc5oc6cc7ccc8ccccc8c7cc6c45)c3)cc2)cc1 ; c1ccc(-c2ccc(N(c3ccc4c(c3)oc3cc5ccc6ccccc6c5cc34)c3ccc4oc5ccccc5c4c3)cc2)cc1 ; c1ccc(-c2ccc(N(c3ccccc3)c3cccc(N(c4ccccc4)c4ccc5c(c4)oc4cc6ccc7ccccc7c6cc45)c3)cc2)cc1]

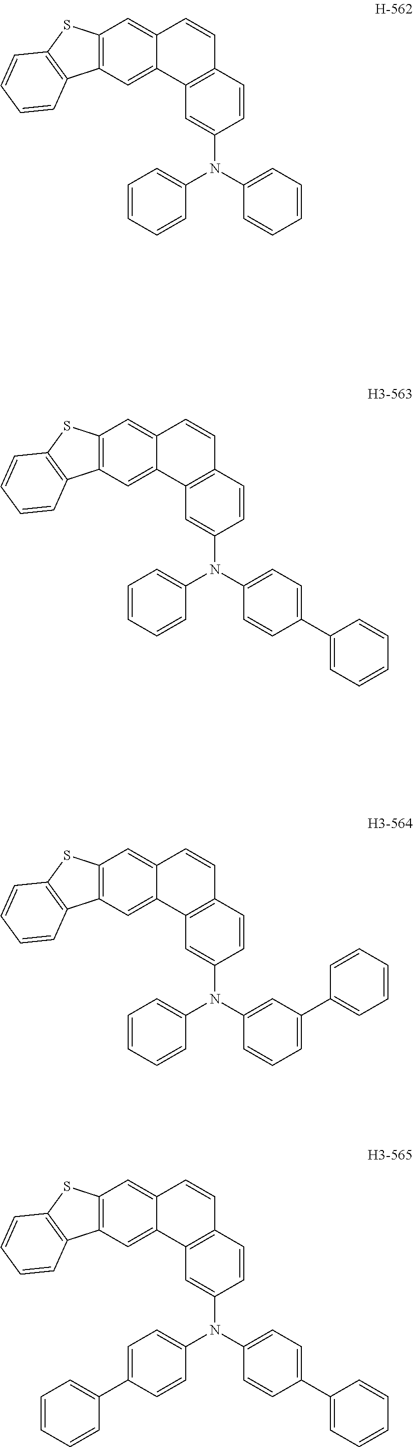 [SMILES: c1ccc(N(c2ccccc2)c2ccc3ccc4cc5sc6ccccc6c5cc4c3c2)cc1 ; c1ccc(-c2ccc(N(c3ccccc3)c3ccc4ccc5cc6sc7ccccc7c6cc5c4c3)cc2)cc1 ; c1ccc(-c2cccc(N(c3ccccc3)c3ccc4ccc5cc6sc7ccccc7c6cc5c4c3)c2)cc1 ; c1ccc(-c2ccc(N(c3ccc(-c4ccccc4)cc3)c3ccc4ccc5cc6sc7ccccc7c6cc5c4c3)cc2)cc1]

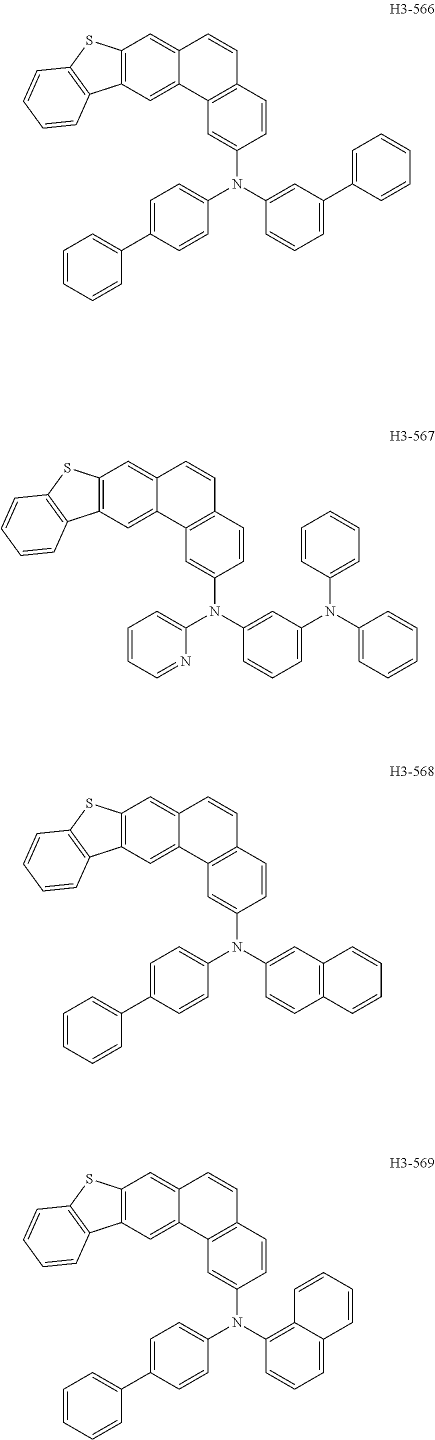 [SMILES: c1ccc(-c2ccc(N(c3cccc(-c4ccccc4)c3)c3ccc4ccc5cc6sc7ccccc7c6cc5c4c3)cc2)cc1 ; c1ccc(N(c2ccccc2)c2cccc(N(c3ccc4ccc5cc6sc7ccccc7c6cc5c4c3)c3ccccn3)c2)cc1 ; c1ccc(-c2ccc(N(c3ccc4ccccc4c3)c3ccc4ccc5cc6sc7ccccc7c6cc5c4c3)cc2)cc1 ; c1ccc(-c2ccc(N(c3ccc4ccc5cc6sc7ccccc7c6cc5c4c3)c3cccc4ccccc34)cc2)cc1]

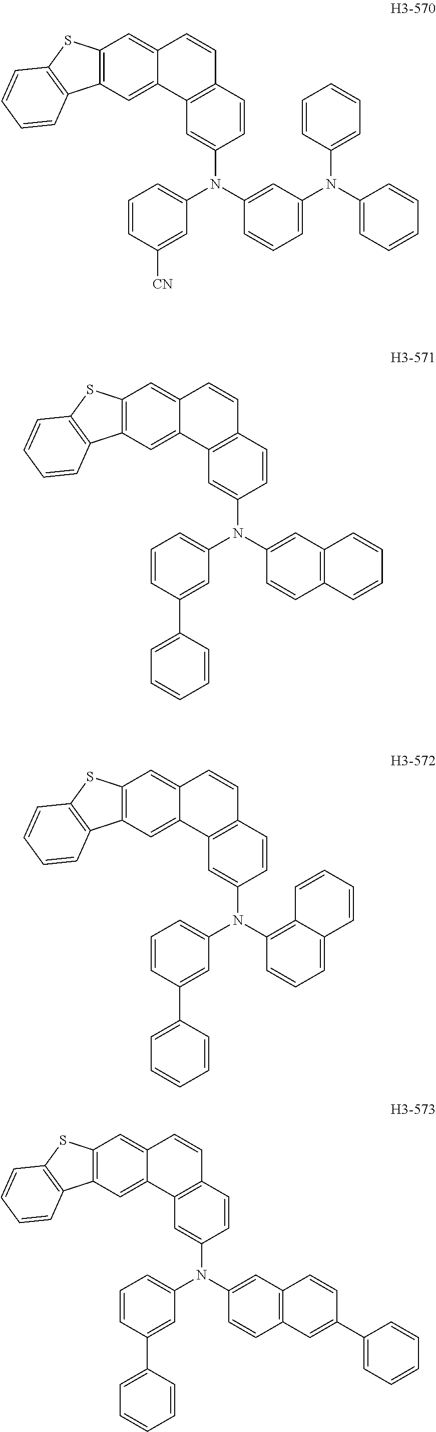 [SMILES: N#Cc1cccc(N(c2cccc(N(c3ccccc3)c3ccccc3)c2)c2ccc3ccc4cc5sc6ccccc6c5cc4c3c2)c1 ; c1ccc(-c2cccc(N(c3ccc4ccccc4c3)c3ccc4ccc5cc6sc7ccccc7c6cc5c4c3)c2)cc1 ; c1ccc(-c2cccc(N(c3ccc4ccc5cc6sc7ccccc7c6cc5c4c3)c3cccc4ccccc34)c2)cc1 ; c1ccc(-c2cccc(N(c3ccc4cc(-c5ccccc5)ccc4c3)c3ccc4ccc5cc6sc7ccccc7c6cc5c4c3)c2)cc1]

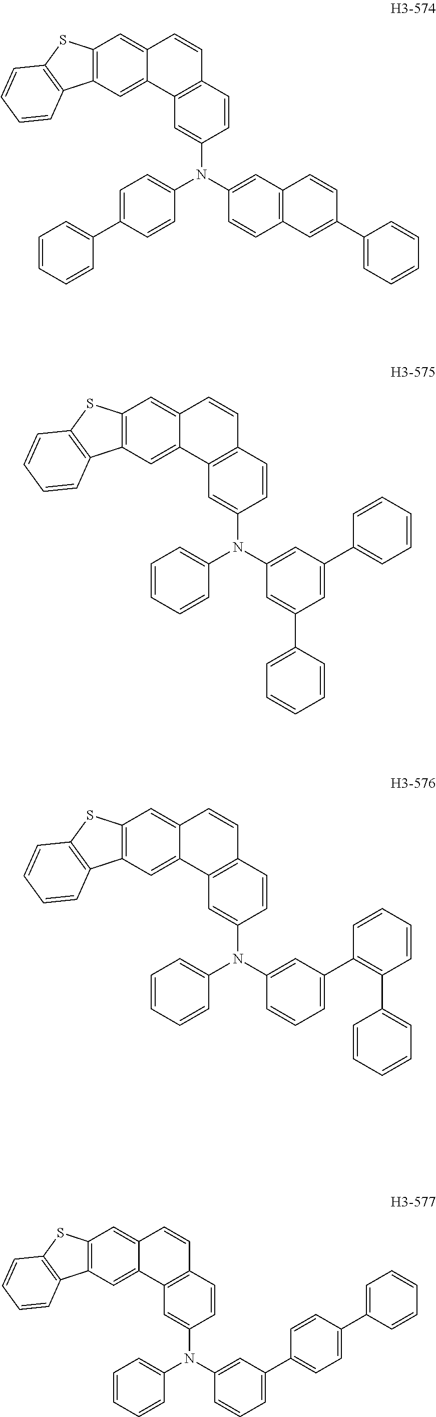 [SMILES: c1ccc(-c2ccc(N(c3ccc4cc(-c5ccccc5)ccc4c3)c3ccc4ccc5cc6sc7ccccc7c6cc5c4c3)cc2)cc1 ; c1ccc(-c2cc(-c3ccccc3)cc(N(c3ccccc3)c3ccc4ccc5cc6sc7ccccc7c6cc5c4c3)c2)cc1 ; c1ccc(-c2ccccc2-c2cccc(N(c3ccccc3)c3ccc4ccc5cc6sc7ccccc7c6cc5c4c3)c2)cc1 ; c1ccc(-c2ccc(-c3cccc(N(c4ccccc4)c4ccc5ccc6cc7sc8ccccc8c7cc6c5c4)c3)cc2)cc1]

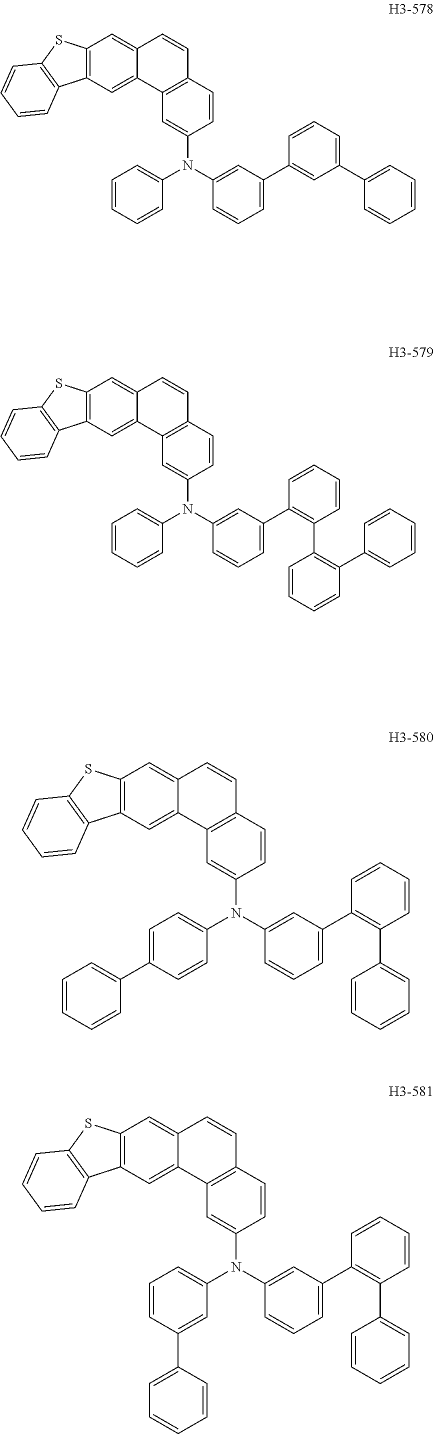 [SMILES: c1ccc(-c2cccc(-c3cccc(N(c4ccccc4)c4ccc5ccc6cc7sc8ccccc8c7cc6c5c4)c3)c2)cc1 ; c1ccc(-c2ccccc2-c2ccccc2-c2cccc(N(c3ccccc3)c3ccc4ccc5cc6sc7ccccc7c6cc5c4c3)c2)cc1 ; c1ccc(-c2ccc(N(c3cccc(-c4ccccc4-c4ccccc4)c3)c3ccc4ccc5cc6sc7ccccc7c6cc5c4c3)cc2)cc1 ; c1ccc(-c2cccc(N(c3cccc(-c4ccccc4-c4ccccc4)c3)c3ccc4ccc5cc6sc7ccccc7c6cc5c4c3)c2)cc1]

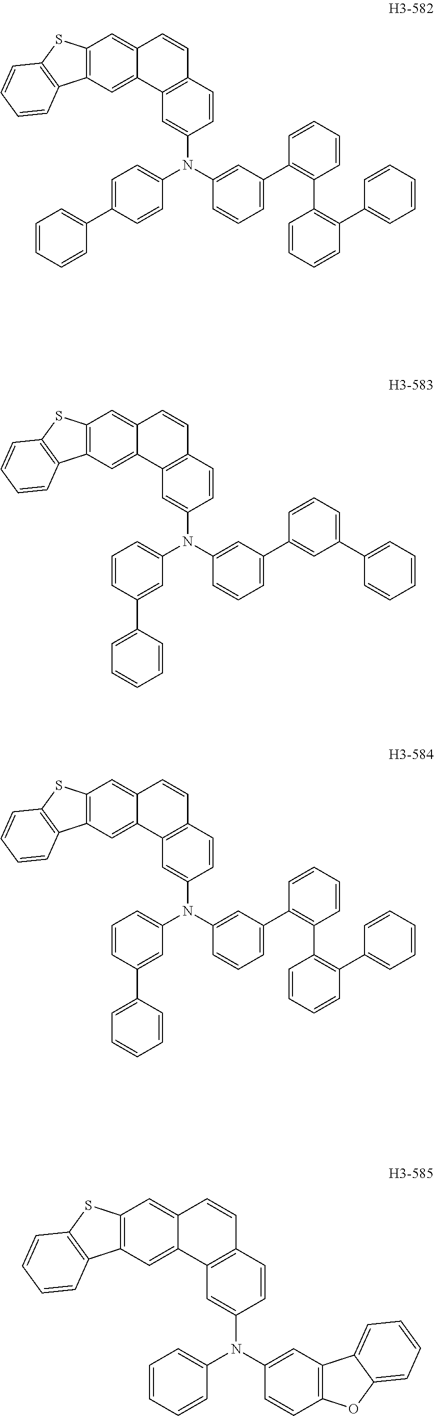 [SMILES: c1ccc(-c2ccc(N(c3cccc(-c4ccccc4-c4ccccc4-c4ccccc4)c3)c3ccc4ccc5cc6sc7ccccc7c6cc5c4c3)cc2)cc1 ; c1ccc(-c2cccc(-c3cccc(N(c4cccc(-c5ccccc5)c4)c4ccc5ccc6cc7sc8ccccc8c7cc6c5c4)c3)c2)cc1 ; c1ccc(-c2cccc(N(c3cccc(-c4ccccc4-c4ccccc4-c4ccccc4)c3)c3ccc4ccc5cc6sc7ccccc7c6cc5c4c3)c2)cc1 ; c1ccc(N(c2ccc3ccc4cc5sc6ccccc6c5cc4c3c2)c2ccc3oc4ccccc4c3c2)cc1]

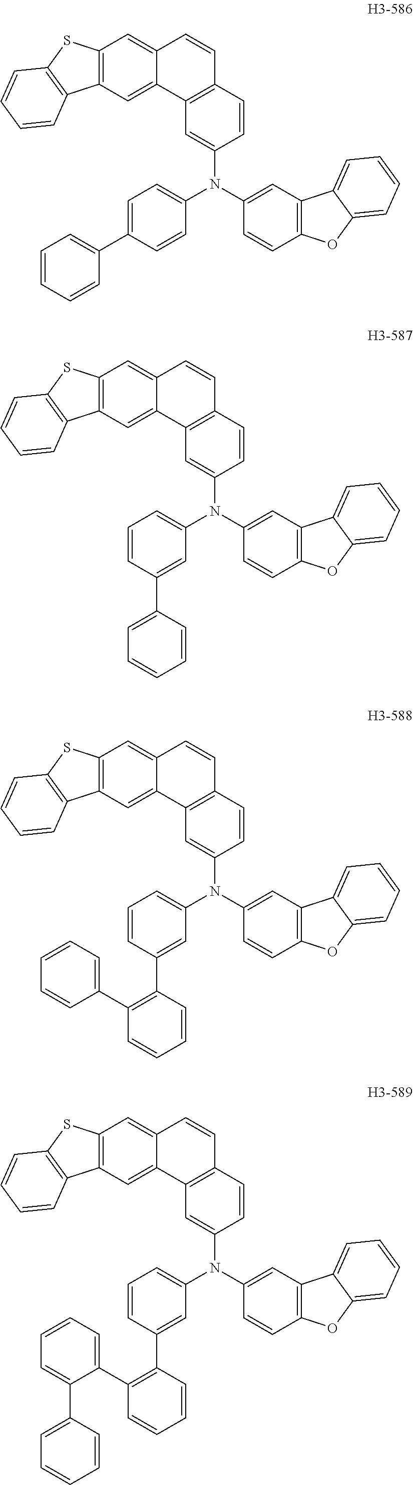 [SMILES: c1ccc(-c2ccc(N(c3ccc4ccc5cc6sc7ccccc7c6cc5c4c3)c3ccc4oc5ccccc5c4c3)cc2)cc1 ; c1ccc(-c2cccc(N(c3ccc4ccc5cc6sc7ccccc7c6cc5c4c3)c3ccc4oc5ccccc5c4c3)c2)cc1 ; c1ccc(-c2ccccc2-c2cccc(N(c3ccc4ccc5cc6sc7ccccc7c6cc5c4c3)c3ccc4oc5ccccc5c4c3)c2)cc1 ; c1ccc(-c2ccccc2-c2ccccc2-c2cccc(N(c3ccc4ccc5cc6sc7ccccc7c6cc5c4c3)c3ccc4oc5ccccc5c4c3)c2)cc1]

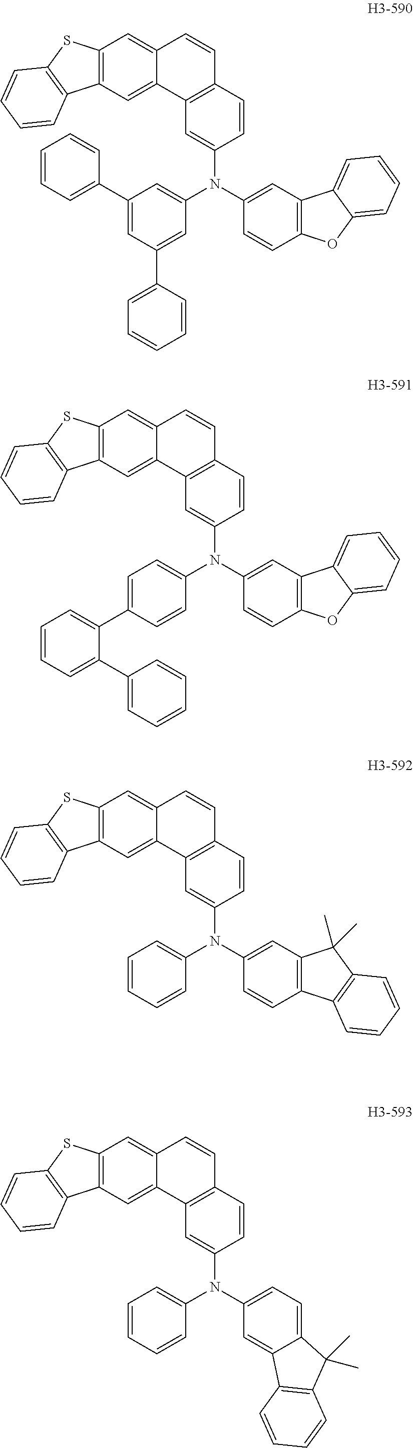 [SMILES: c1ccc(-c2cc(-c3ccccc3)cc(N(c3ccc4ccc5cc6sc7ccccc7c6cc5c4c3)c3ccc4oc5ccccc5c4c3)c2)cc1 ; c1ccc(-c2ccccc2-c2ccc(N(c3ccc4ccc5cc6sc7ccccc7c6cc5c4c3)c3ccc4oc5ccccc5c4c3)cc2)cc1 ; CC1(C)c2ccccc2-c2ccc(N(c3ccccc3)c3ccc4ccc5cc6sc7ccccc7c6cc5c4c3)cc21 ; CC1(C)c2ccccc2-c2cc(N(c3ccccc3)c3ccc4ccc5cc6sc7ccccc7c6cc5c4c3)ccc21]

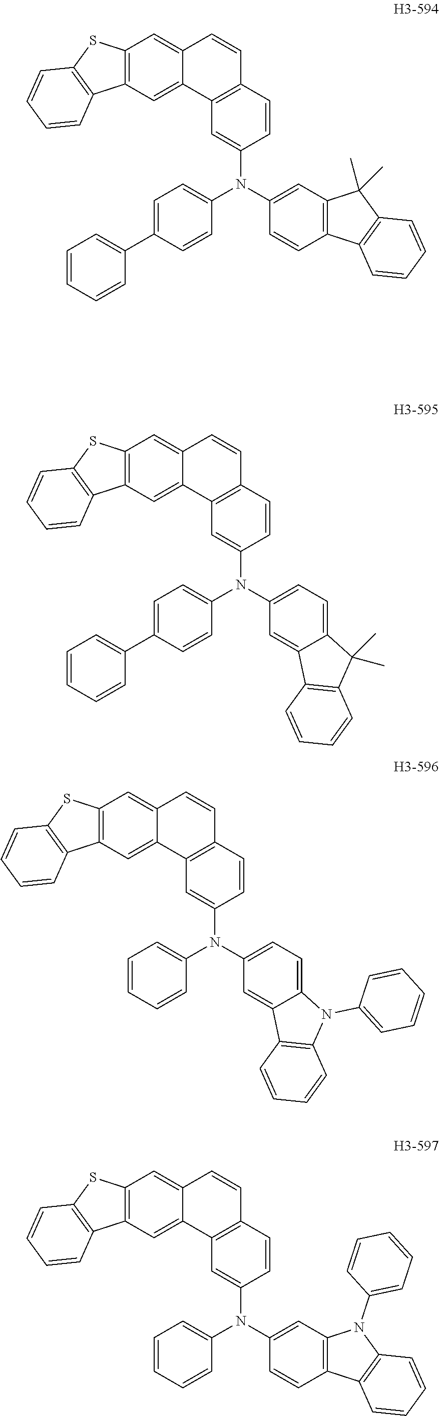 [SMILES: CC1(C)c2ccccc2-c2ccc(N(c3ccc(-c4ccccc4)cc3)c3ccc4ccc5cc6sc7ccccc7c6cc5c4c3)cc21 ; CC1(C)c2ccccc2-c2cc(N(c3ccc(-c4ccccc4)cc3)c3ccc4ccc5cc6sc7ccccc7c6cc5c4c3)ccc21 ; c1ccc(N(c2ccc3ccc4cc5sc6ccccc6c5cc4c3c2)c2ccc3c(c2)c2ccccc2n3-c2ccccc2)cc1 ; c1ccc(N(c2ccc3ccc4cc5sc6ccccc6c5cc4c3c2)c2ccc3c4ccccc4n(-c4ccccc4)c3c2)cc1]

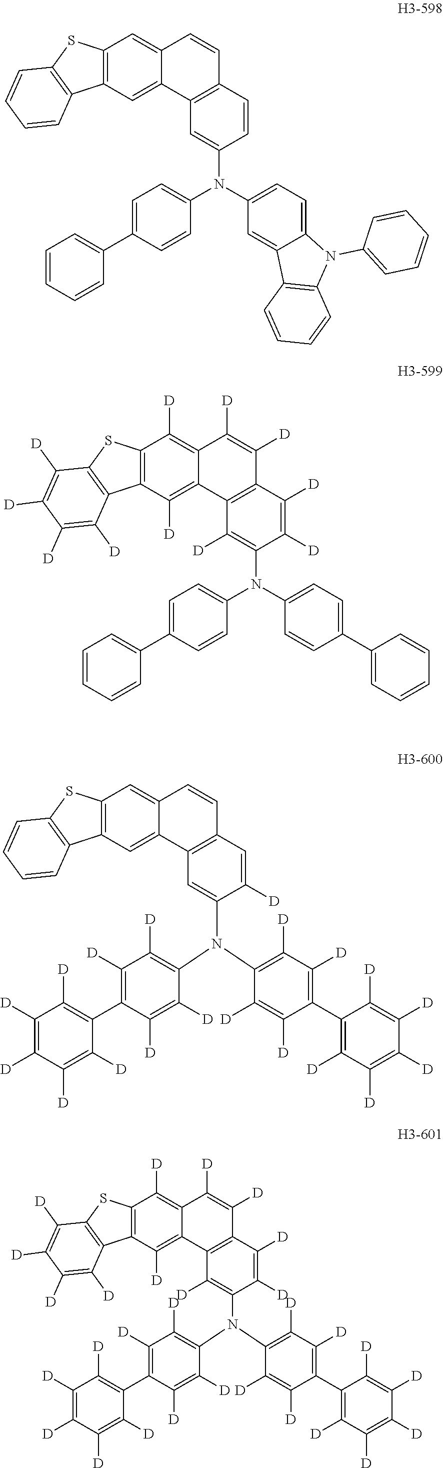

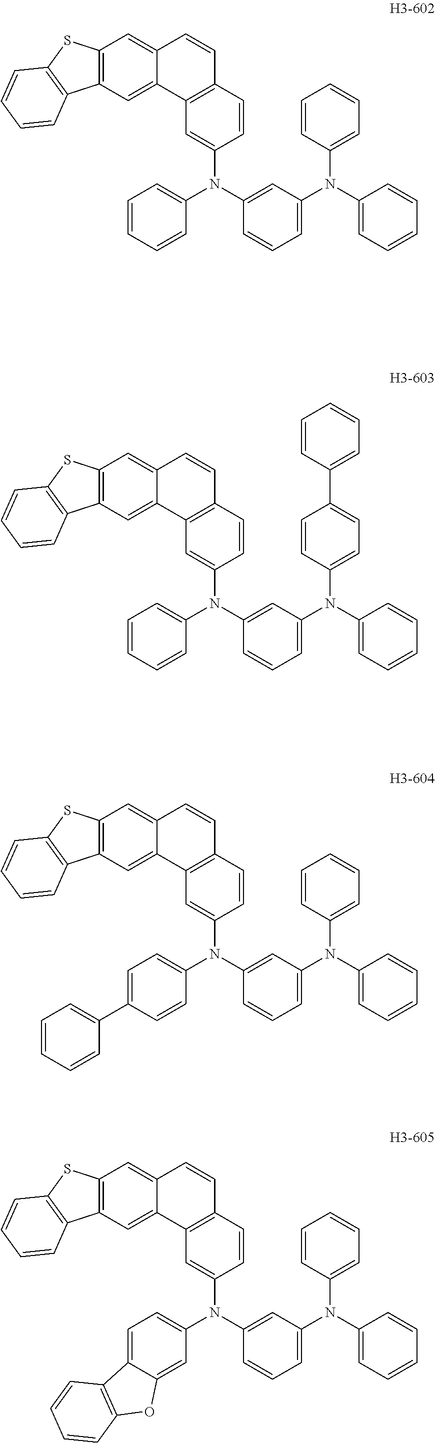 [SMILES: c1ccc(N(c2ccccc2)c2cccc(N(c3ccccc3)c3ccc4ccc5cc6sc7ccccc7c6cc5c4c3)c2)cc1 ; c1ccc(-c2ccc(N(c3ccccc3)c3cccc(N(c4ccccc4)c4ccc5ccc6cc7sc8ccccc8c7cc6c5c4)c3)cc2)cc1 ; c1ccc(-c2ccc(N(c3cccc(N(c4ccccc4)c4ccccc4)c3)c3ccc4ccc5cc6sc7ccccc7c6cc5c4c3)cc2)cc1 ; c1ccc(N(c2ccccc2)c2cccc(N(c3ccc4c(c3)oc3ccccc34)c3ccc4ccc5cc6sc7ccccc7c6cc5c4c3)c2)cc1]

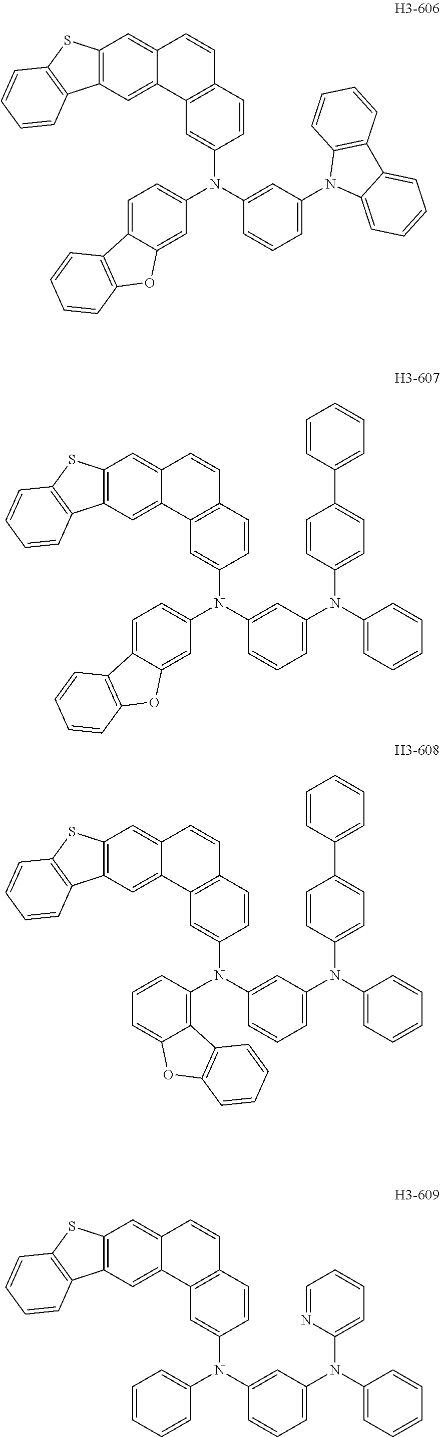 [SMILES: c1cc(N(c2ccc3c(c2)oc2ccccc23)c2ccc3ccc4cc5sc6ccccc6c5cc4c3c2)cc(-n2c3ccccc3c3ccccc32)c1 ; c1ccc(-c2ccc(N(c3ccccc3)c3cccc(N(c4ccc5c(c4)oc4ccccc45)c4ccc5ccc6cc7sc8ccccc8c7cc6c5c4)c3)cc2)cc1 ; c1ccc(-c2ccc(N(c3ccccc3)c3cccc(N(c4ccc5ccc6cc7sc8ccccc8c7cc6c5c4)c4cccc5oc6ccccc6c45)c3)cc2)cc1 ; c1ccc(N(c2cccc(N(c3ccccc3)c3ccccn3)c2)c2ccc3ccc4cc5sc6ccccc6c5cc4c3c2)cc1]

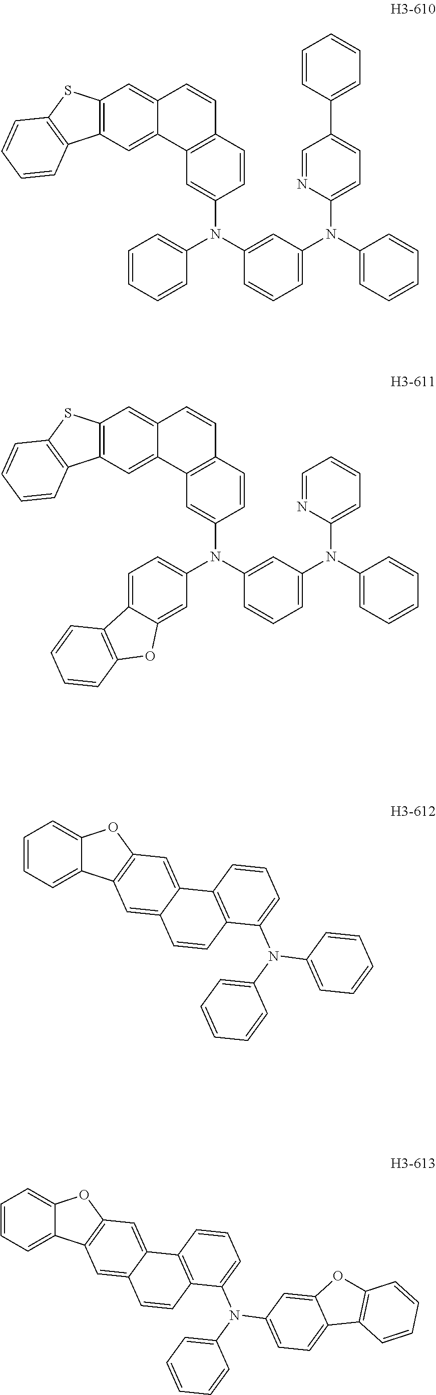 [SMILES: c1ccc(-c2ccc(N(c3ccccc3)c3cccc(N(c4ccccc4)c4ccc5ccc6cc7sc8ccccc8c7cc6c5c4)c3)nc2)cc1 ; c1ccc(N(c2cccc(N(c3ccc4c(c3)oc3ccccc34)c3ccc4ccc5cc6sc7ccccc7c6cc5c4c3)c2)c2ccccn2)cc1 ; c1ccc(N(c2ccccc2)c2cccc3c2ccc2cc4c(cc23)oc2ccccc24)cc1 ; c1ccc(N(c2ccc3c(c2)oc2ccccc23)c2cccc3c2ccc2cc4c(cc23)oc2ccccc24)cc1]

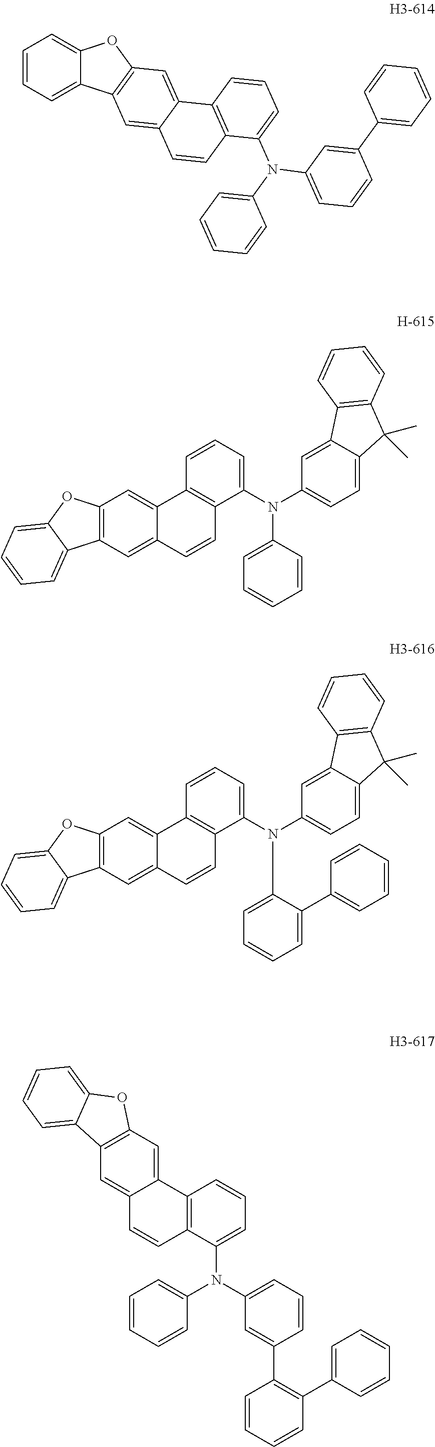 [SMILES: c1ccc(-c2cccc(N(c3ccccc3)c3cccc4c3ccc3cc5c(cc34)oc3ccccc35)c2)cc1 ; CC1(C)c2ccccc2-c2cc(N(c3ccccc3)c3cccc4c3ccc3cc5c(cc34)oc3ccccc35)ccc21 ; CC1(C)c2ccccc2-c2cc(N(c3ccccc3-c3ccccc3)c3cccc4c3ccc3cc5c(cc34)oc3ccccc35)ccc21 ; c1ccc(-c2ccccc2-c2cccc(N(c3ccccc3)c3cccc4c3ccc3cc5c(cc34)oc3ccccc35)c2)cc1]

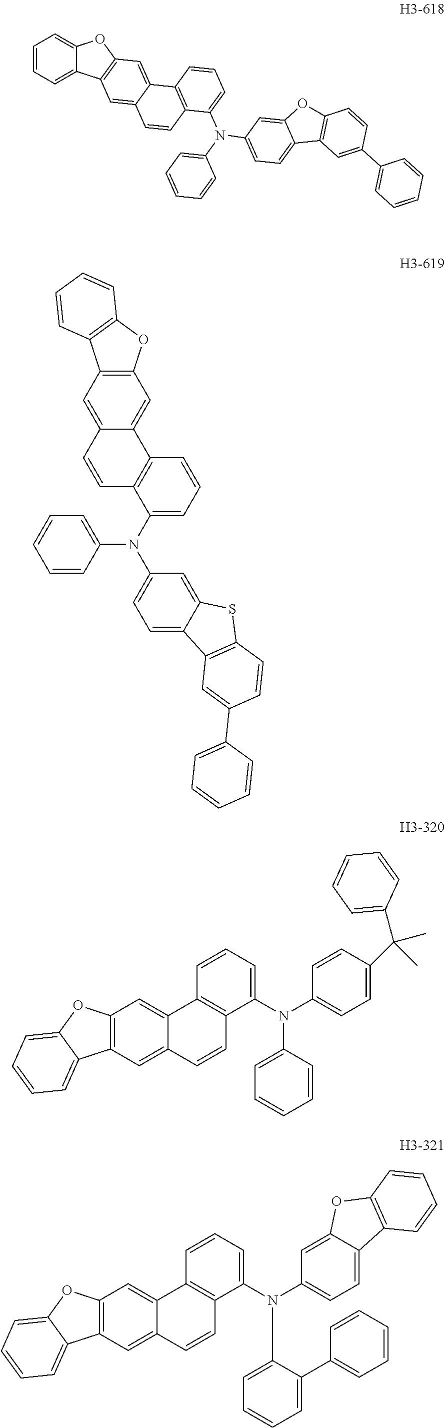 [SMILES: c1ccc(-c2ccc3oc4cc(N(c5ccccc5)c5cccc6c5ccc5cc7c(cc56)oc5ccccc57)ccc4c3c2)cc1 ; c1ccc(-c2ccc3sc4cc(N(c5ccccc5)c5cccc6c5ccc5cc7c(cc56)oc5ccccc57)ccc4c3c2)cc1 ; CC(C)(c1ccccc1)c1ccc(N(c2ccccc2)c2cccc3c2ccc2cc4c(cc23)oc2ccccc24)cc1 ; c1ccc(-c2ccccc2N(c2ccc3c(c2)oc2ccccc23)c2cccc3c2ccc2cc4c(cc23)oc2ccccc24)cc1]

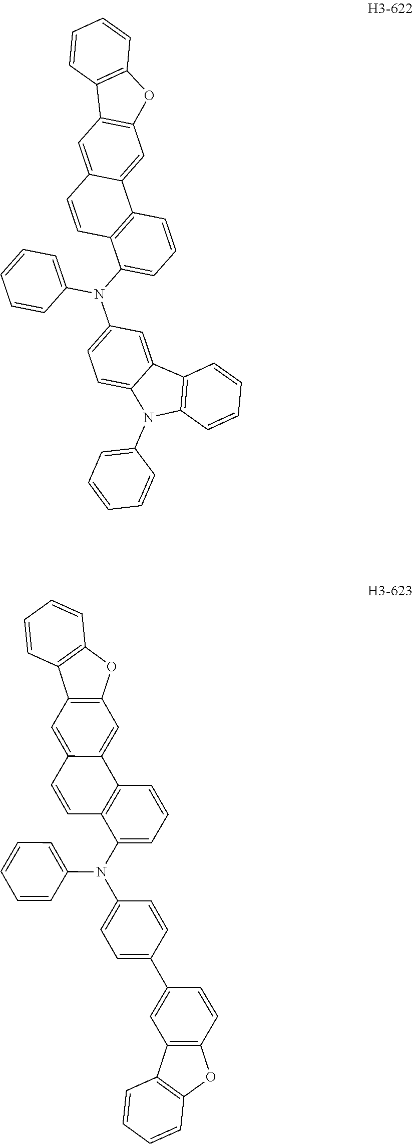 [SMILES: c1ccc(N(c2ccc3c(c2)c2ccccc2n3-c2ccccc2)c2cccc3c2ccc2cc4c(cc23)oc2ccccc24)cc1 ; c1ccc(N(c2ccc(-c3ccc4oc5ccccc5c4c3)cc2)c2cccc3c2ccc2cc4c(cc23)oc2ccccc24)cc1]

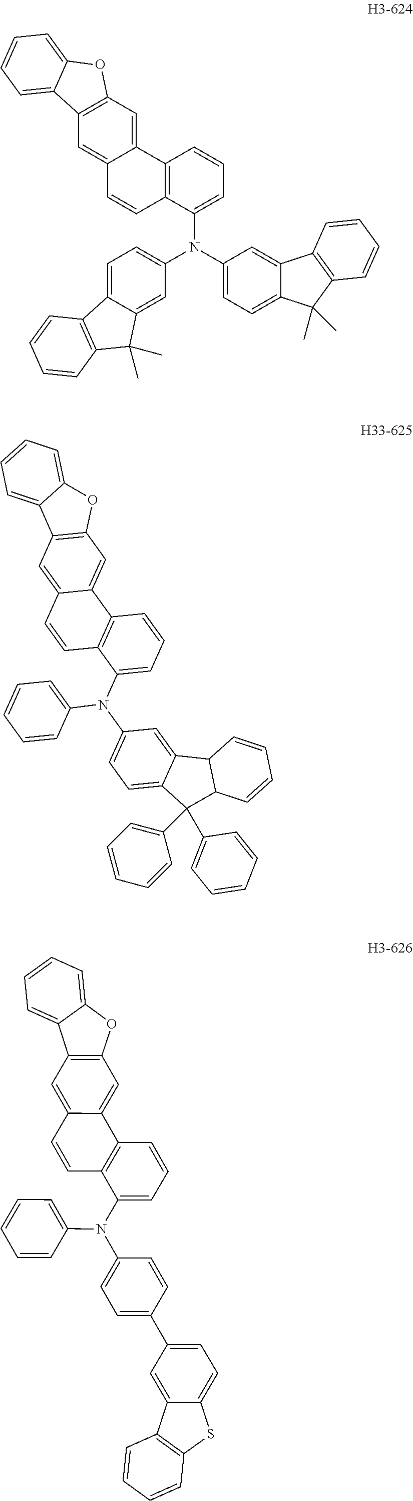 [SMILES: CC1(C)c2ccccc2-c2cc(N(c3ccc4c(c3)C(C)(C)c3ccccc3-4)c3cccc4c3ccc3cc5c(cc34)oc3ccccc35)ccc21 ; C1=CC2c3cc(N(c4ccccc4)c4cccc5c4ccc4cc6c(cc45)oc4ccccc46)ccc3C(c3ccccc3)(c3ccccc3)C2C=C1 ; c1ccc(N(c2ccc(-c3ccc4sc5ccccc5c4c3)cc2)c2cccc3c2ccc2cc4c(cc23)oc2ccccc24)cc1]

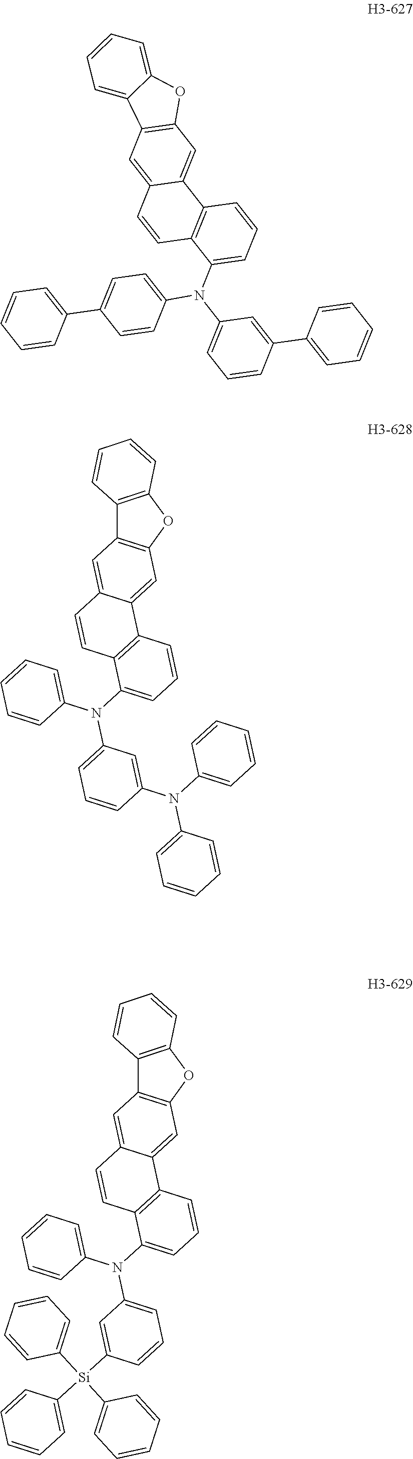 [SMILES: c1ccc(-c2ccc(N(c3cccc(-c4ccccc4)c3)c3cccc4c3ccc3cc5c(cc34)oc3ccccc35)cc2)cc1 ; c1ccc(N(c2ccccc2)c2cccc(N(c3ccccc3)c3cccc4c3ccc3cc5c(cc34)oc3ccccc35)c2)cc1 ; c1ccc(N(c2cccc([Si](c3ccccc3)(c3ccccc3)c3ccccc3)c2)c2cccc3c2ccc2cc4c(cc23)oc2ccccc24)cc1]

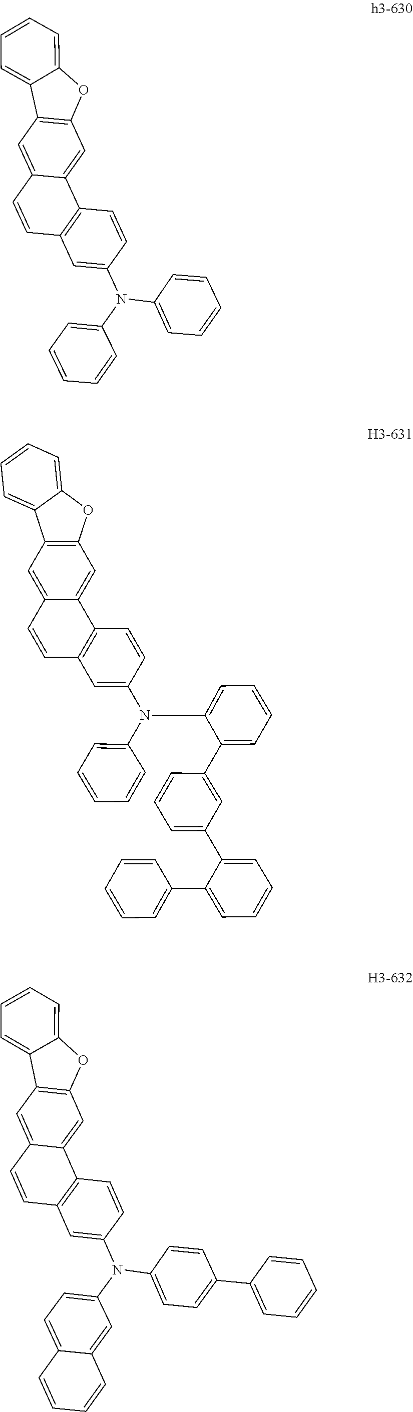 [SMILES: c1ccc(N(c2ccccc2)c2ccc3c(ccc4cc5c(cc43)oc3ccccc35)c2)cc1 ; c1ccc(-c2ccccc2-c2cccc(-c3ccccc3N(c3ccccc3)c3ccc4c(ccc5cc6c(cc54)oc4ccccc46)c3)c2)cc1 ; c1ccc(-c2ccc(N(c3ccc4ccccc4c3)c3ccc4c(ccc5cc6c(cc54)oc4ccccc46)c3)cc2)cc1]

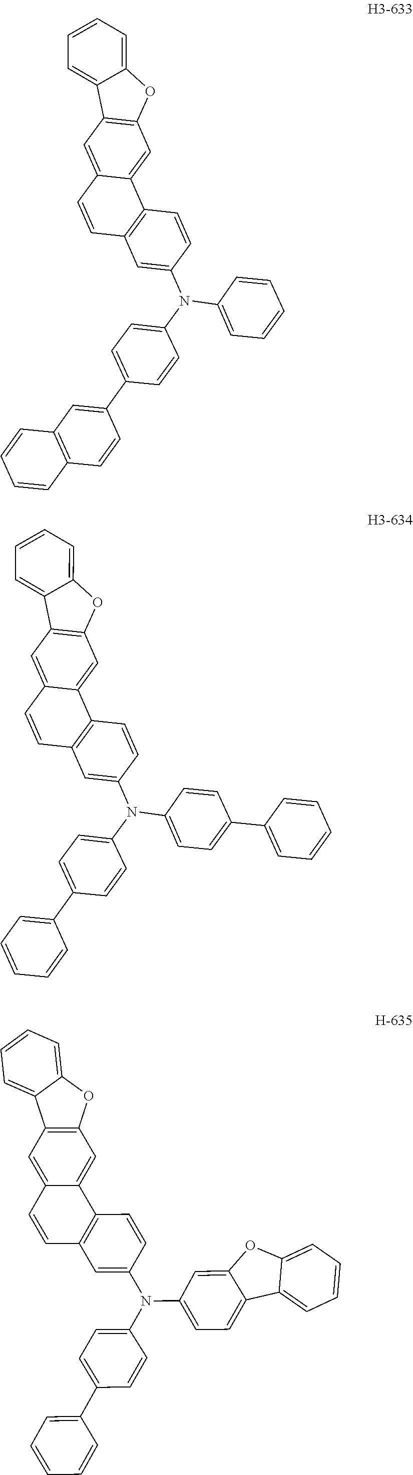

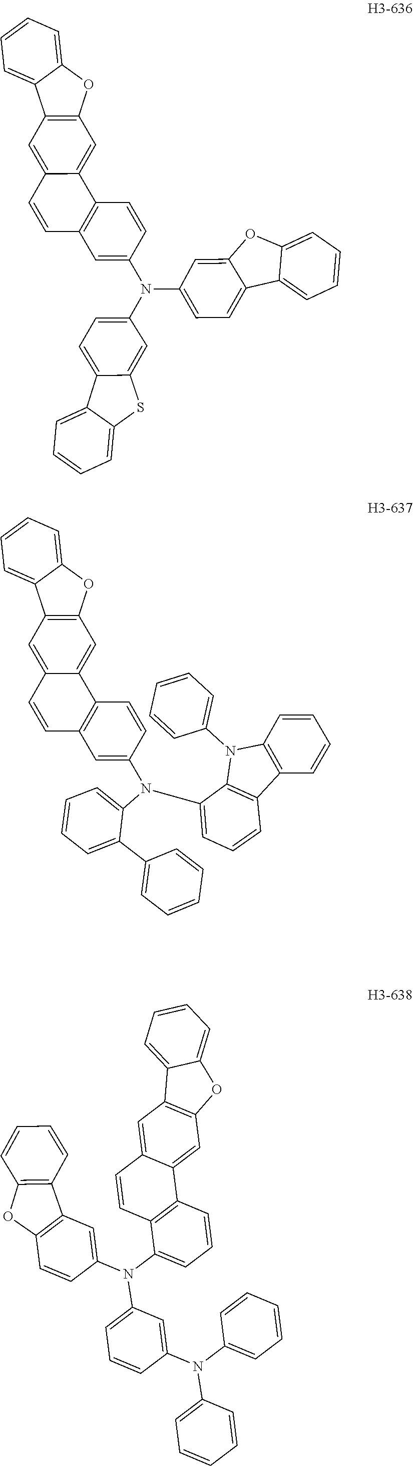 [SMILES: c1ccc2c(c1)oc1cc(N(c3ccc4c(ccc5cc6c(cc54)oc4ccccc46)c3)c3ccc4c(c3)sc3ccccc34)ccc12 ; c1ccc(-c2ccccc2N(c2ccc3c(ccc4cc5c(cc43)oc3ccccc35)c2)c2cccc3c4ccccc4n(-c4ccccc4)c23)cc1 ; c1ccc(N(c2ccccc2)c2cccc(N(c3ccc4oc5ccccc5c4c3)c3cccc4c3ccc3cc5c(cc34)oc3ccccc35)c2)cc1]

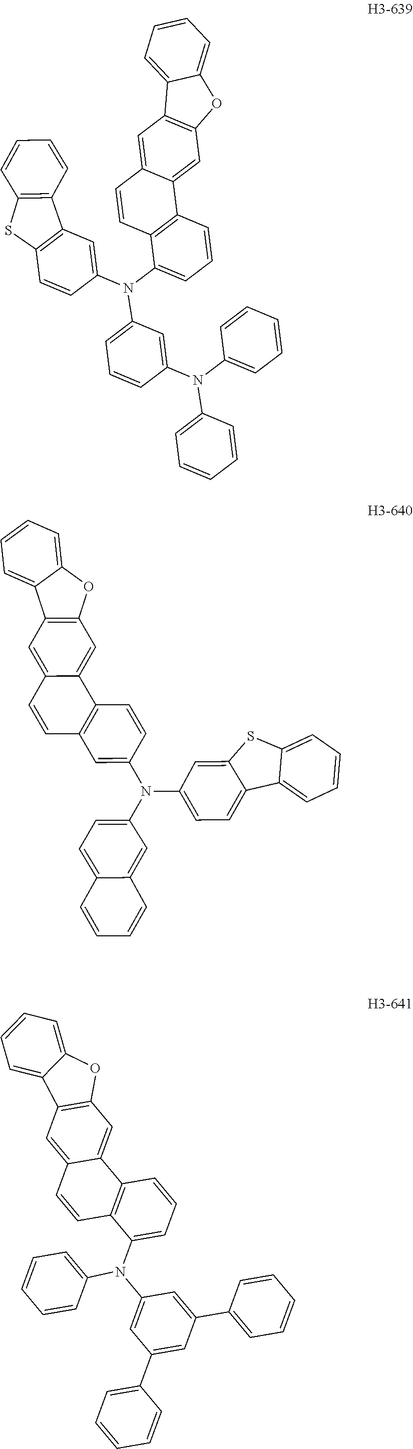 [SMILES: c1ccc(N(c2ccccc2)c2cccc(N(c3ccc4sc5ccccc5c4c3)c3cccc4c3ccc3cc5c(cc34)oc3ccccc35)c2)cc1 ; c1ccc2cc(N(c3ccc4c(ccc5cc6c(cc54)oc4ccccc46)c3)c3ccc4c(c3)sc3ccccc34)ccc2c1 ; c1ccc(-c2cc(-c3ccccc3)cc(N(c3ccccc3)c3cccc4c3ccc3cc5c(cc34)oc3ccccc35)c2)cc1]

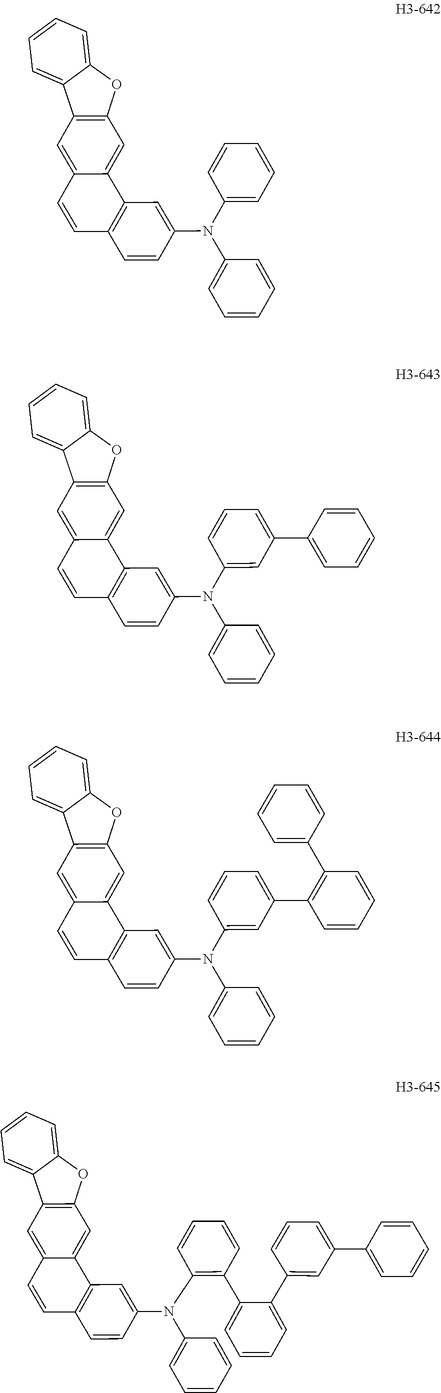 [SMILES: c1ccc(N(c2ccccc2)c2ccc3ccc4cc5c(cc4c3c2)oc2ccccc25)cc1 ; c1ccc(-c2cccc(N(c3ccccc3)c3ccc4ccc5cc6c(cc5c4c3)oc3ccccc36)c2)cc1 ; c1ccc(-c2ccccc2-c2cccc(N(c3ccccc3)c3ccc4ccc5cc6c(cc5c4c3)oc3ccccc36)c2)cc1 ; c1ccc(-c2cccc(-c3ccccc3-c3ccccc3N(c3ccccc3)c3ccc4ccc5cc6c(cc5c4c3)oc3ccccc36)c2)cc1]

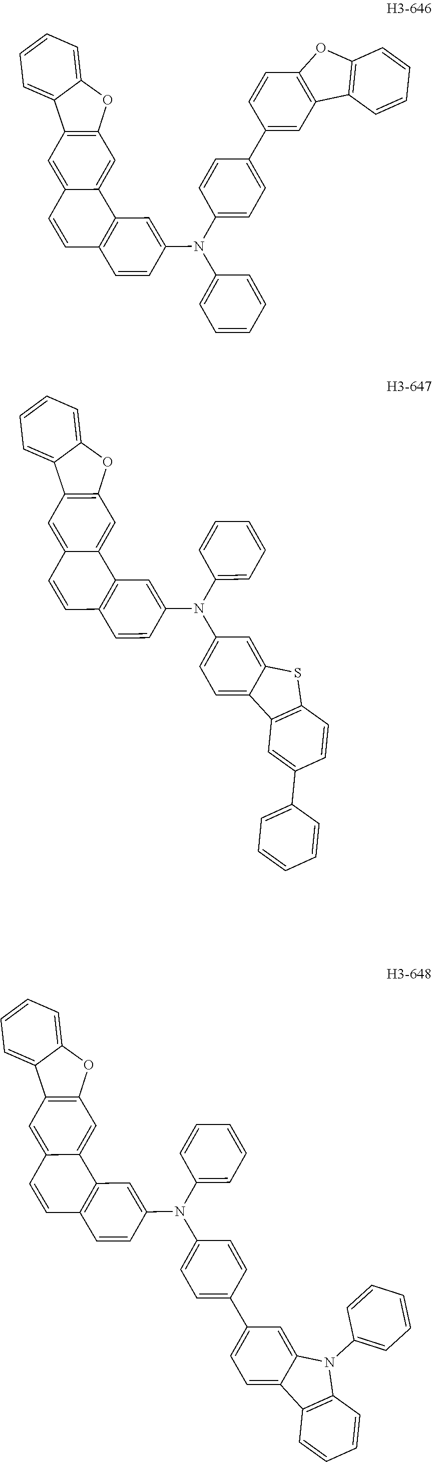 [SMILES: c1ccc(N(c2ccc(-c3ccc4oc5ccccc5c4c3)cc2)c2ccc3ccc4cc5c(cc4c3c2)oc2ccccc25)cc1 ; c1ccc(-c2ccc3sc4cc(N(c5ccccc5)c5ccc6ccc7cc8c(cc7c6c5)oc5ccccc58)ccc4c3c2)cc1 ; c1ccc(N(c2ccc(-c3ccc4c5ccccc5n(-c5ccccc5)c4c3)cc2)c2ccc3ccc4cc5c(cc4c3c2)oc2ccccc25)cc1]

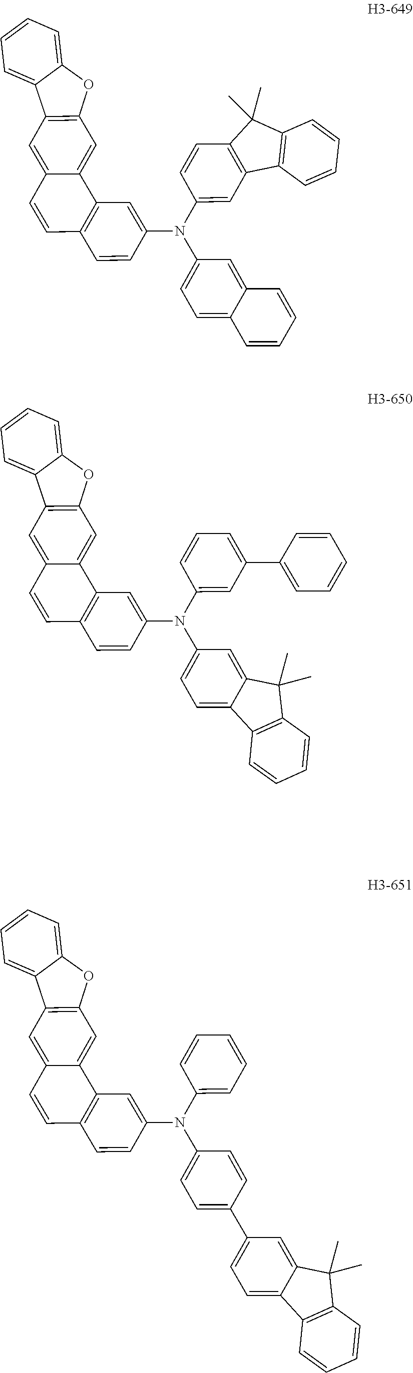 [SMILES: CC1(C)c2ccccc2-c2cc(N(c3ccc4ccccc4c3)c3ccc4ccc5cc6c(cc5c4c3)oc3ccccc36)ccc21 ; CC1(C)c2ccccc2-c2ccc(N(c3cccc(-c4ccccc4)c3)c3ccc4ccc5cc6c(cc5c4c3)oc3ccccc36)cc21 ; CC1(C)c2ccccc2-c2ccc(-c3ccc(N(c4ccccc4)c4ccc5ccc6cc7c(cc6c5c4)oc4ccccc47)cc3)cc21]

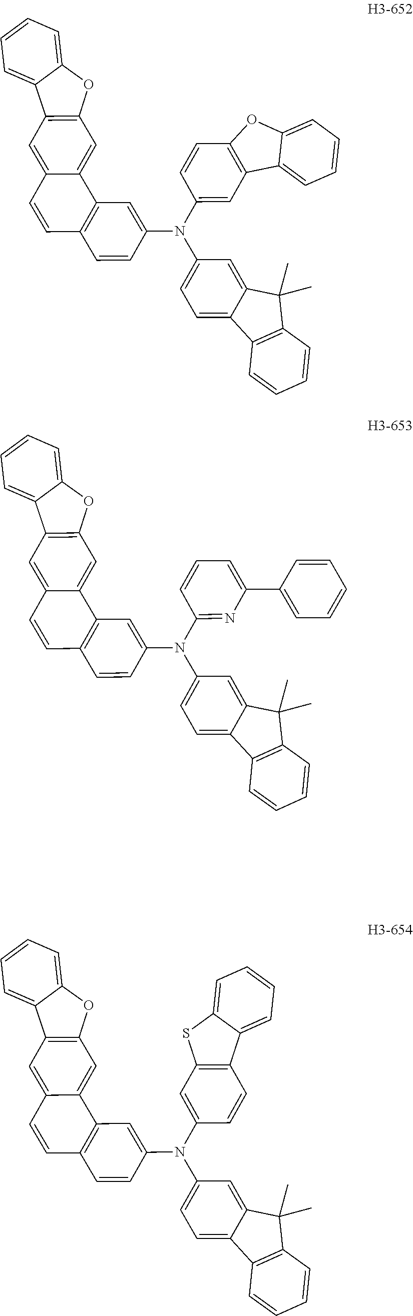 [SMILES: CC1(C)c2ccccc2-c2ccc(N(c3ccc4ccc5cc6c(cc5c4c3)oc3ccccc36)c3ccc4oc5ccccc5c4c3)cc21 ; CC1(C)c2ccccc2-c2ccc(N(c3ccc4ccc5cc6c(cc5c4c3)oc3ccccc36)c3cccc(-c4ccccc4)n3)cc21 ; CC1(C)c2ccccc2-c2ccc(N(c3ccc4c(c3)sc3ccccc34)c3ccc4ccc5cc6c(cc5c4c3)oc3ccccc36)cc21]

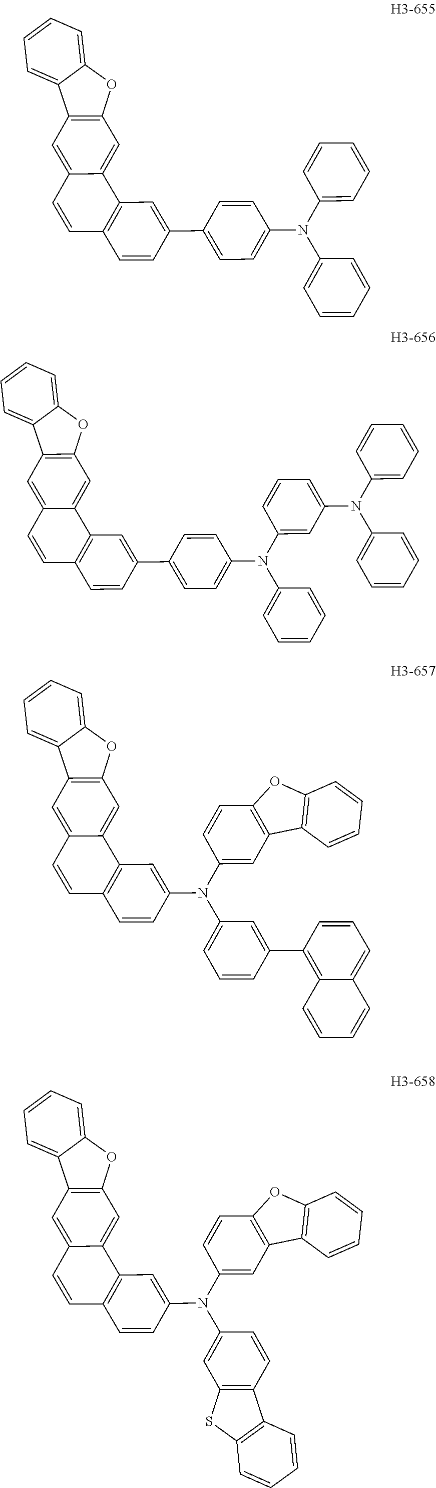 [SMILES: c1ccc(N(c2ccccc2)c2ccc(-c3ccc4ccc5cc6c(cc5c4c3)oc3ccccc36)cc2)cc1 ; c1ccc(N(c2ccccc2)c2cccc(N(c3ccccc3)c3ccc(-c4ccc5ccc6cc7c(cc6c5c4)oc4ccccc47)cc3)c2)cc1 ; c1cc(-c2cccc3ccccc23)cc(N(c2ccc3ccc4cc5c(cc4c3c2)oc2ccccc25)c2ccc3oc4ccccc4c3c2)c1 ; c1ccc2c(c1)oc1ccc(N(c3ccc4c(c3)sc3ccccc34)c3ccc4ccc5cc6c(cc5c4c3)oc3ccccc36)cc12]

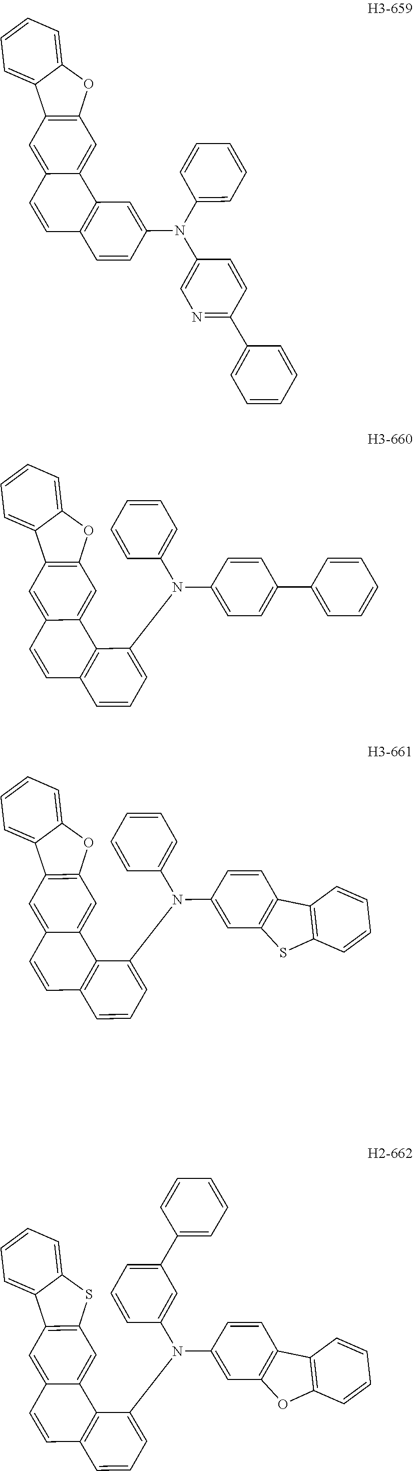 [SMILES: c1ccc(-c2ccc(N(c3ccccc3)c3ccc4ccc5cc6c(cc5c4c3)oc3ccccc36)cn2)cc1 ; c1ccc(-c2ccc(N(c3ccccc3)c3cccc4ccc5cc6c(cc5c34)oc3ccccc36)cc2)cc1 ; c1ccc(N(c2ccc3c(c2)sc2ccccc23)c2cccc3ccc4cc5c(cc4c23)oc2ccccc25)cc1 ; c1ccc(-c2cccc(N(c3ccc4c(c3)oc3ccccc34)c3cccc4ccc5cc6c(cc5c34)sc3ccccc36)c2)cc1]

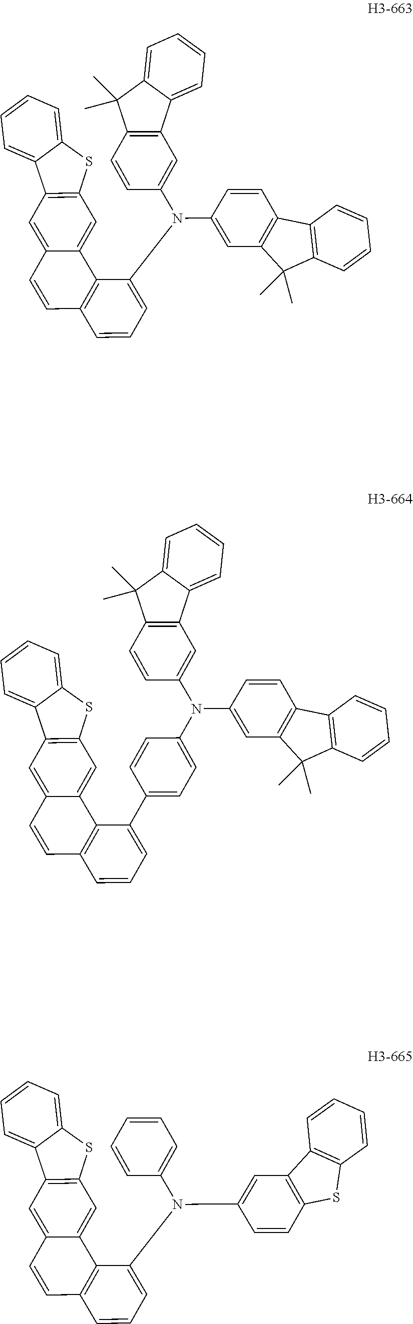 [SMILES: CC1(C)c2ccccc2-c2cc(N(c3ccc4c(c3)C(C)(C)c3ccccc3-4)c3cccc4ccc5cc6c(cc5c34)sc3ccccc36)ccc21 ; CC1(C)c2ccccc2-c2cc(N(c3ccc(-c4cccc5ccc6cc7c(cc6c45)sc4ccccc47)cc3)c3ccc4c(c3)C(C)(C)c3ccccc3-4)ccc21 ; c1ccc(N(c2ccc3sc4ccccc4c3c2)c2cccc3ccc4cc5c(cc4c23)sc2ccccc25)cc1]

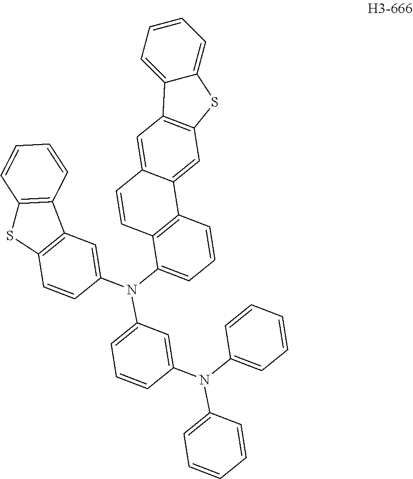 [SMILES: c1ccc(N(c2ccccc2)c2cccc(N(c3ccc4sc5ccccc5c4c3)c3cccc4c3ccc3cc5c(cc34)sc3ccccc35)c2)cc1]

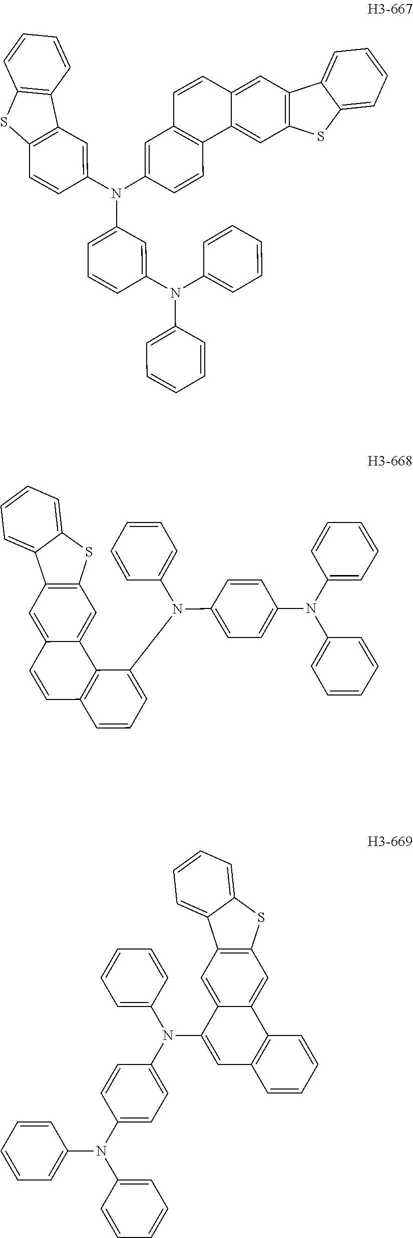 [SMILES: c1ccc(N(c2ccccc2)c2cccc(N(c3ccc4c(ccc5cc6c(cc54)sc4ccccc46)c3)c3ccc4sc5ccccc5c4c3)c2)cc1 ; c1ccc(N(c2ccccc2)c2ccc(N(c3ccccc3)c3cccc4ccc5cc6c(cc5c34)sc3ccccc36)cc2)cc1 ; c1ccc(N(c2ccccc2)c2ccc(N(c3ccccc3)c3cc4ccccc4c4cc5sc6ccccc6c5cc34)cc2)cc1]

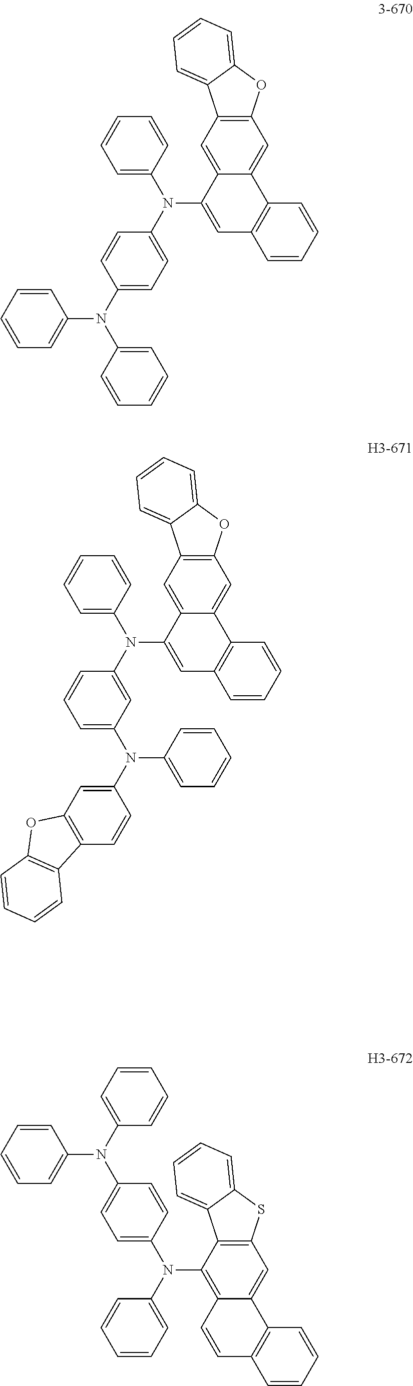 [SMILES: c1ccc(N(c2ccccc2)c2ccc(N(c3ccccc3)c3cc4ccccc4c4cc5oc6ccccc6c5cc34)cc2)cc1 ; c1ccc(N(c2cccc(N(c3ccccc3)c3cc4ccccc4c4cc5oc6ccccc6c5cc34)c2)c2ccc3c(c2)oc2ccccc23)cc1 ; c1ccc(N(c2ccccc2)c2ccc(N(c3ccccc3)c3c4ccc5ccccc5c4cc4sc5ccccc5c34)cc2)cc1]

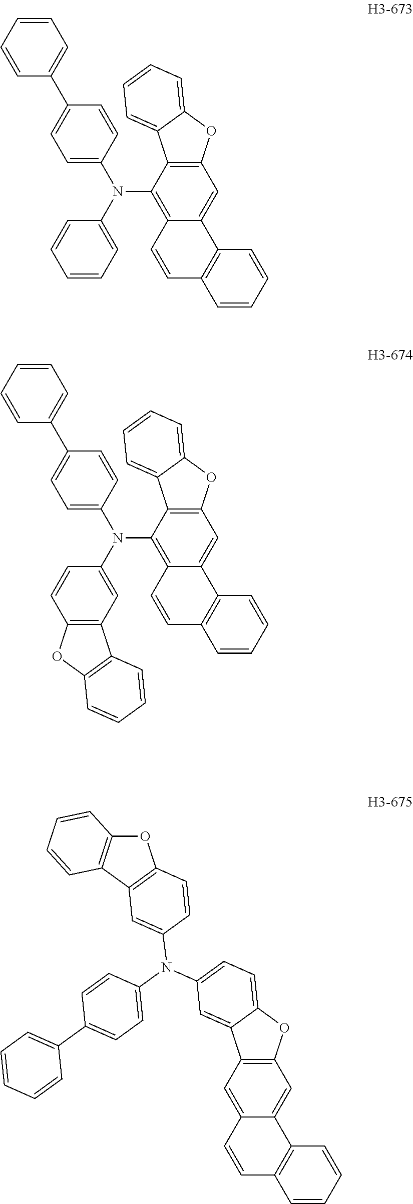 [SMILES: c1ccc(-c2ccc(N(c3ccccc3)c3c4ccc5ccccc5c4cc4oc5ccccc5c34)cc2)cc1 ; c1ccc(-c2ccc(N(c3ccc4oc5ccccc5c4c3)c3c4ccc5ccccc5c4cc4oc5ccccc5c34)cc2)cc1 ; c1ccc(-c2ccc(N(c3ccc4oc5ccccc5c4c3)c3ccc4oc5cc6c(ccc7ccccc76)cc5c4c3)cc2)cc1]

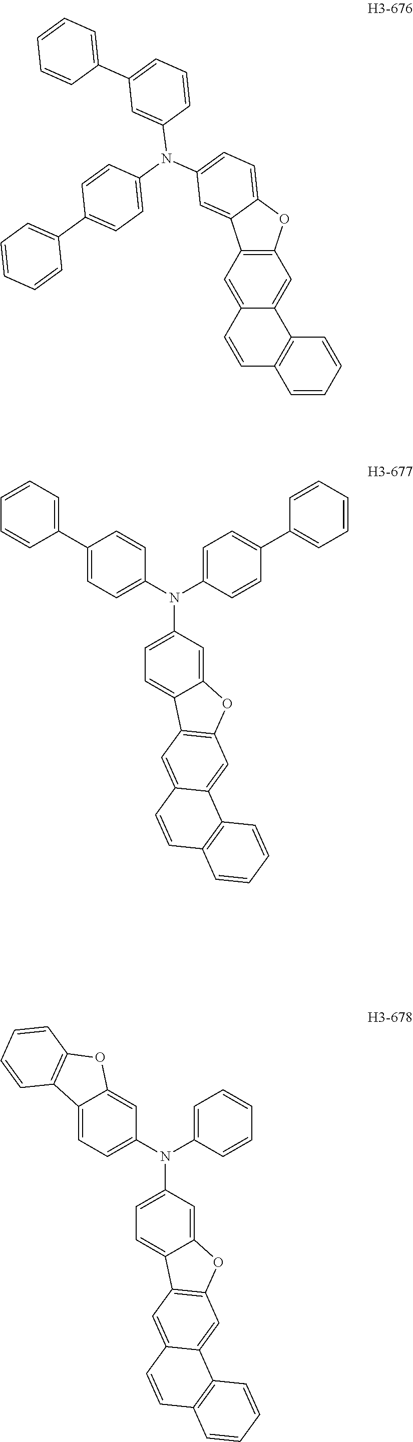 [SMILES: c1ccc(-c2ccc(N(c3cccc(-c4ccccc4)c3)c3ccc4oc5cc6c(ccc7ccccc76)cc5c4c3)cc2)cc1 ; c1ccc(-c2ccc(N(c3ccc(-c4ccccc4)cc3)c3ccc4c(c3)oc3cc5c(ccc6ccccc65)cc34)cc2)cc1 ; c1ccc(N(c2ccc3c(c2)oc2ccccc23)c2ccc3c(c2)oc2cc4c(ccc5ccccc54)cc23)cc1]

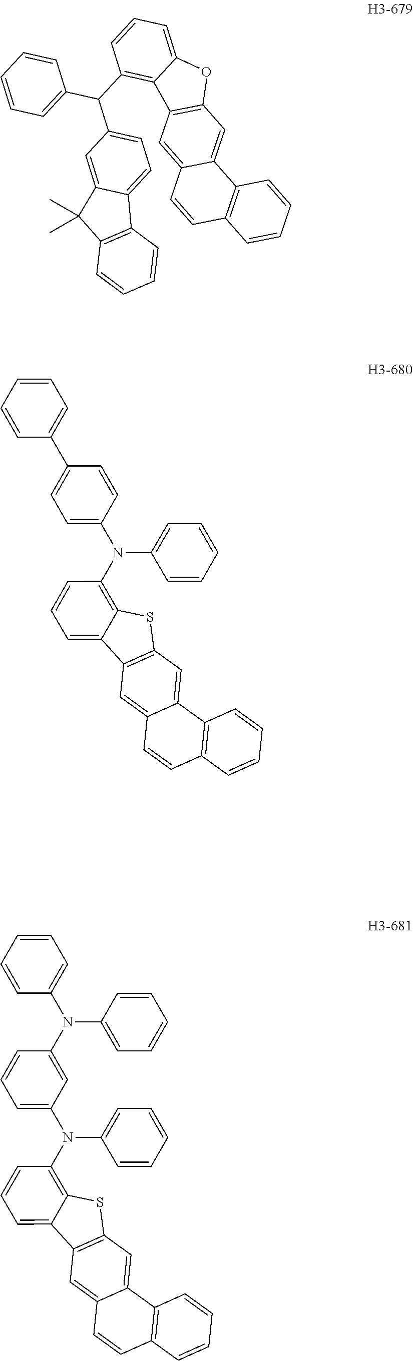 [SMILES: CC1(C)c2ccccc2-c2ccc(C(c3ccccc3)c3cccc4oc5cc6c(ccc7ccccc76)cc5c34)cc21 ; c1ccc(-c2ccc(N(c3ccccc3)c3cccc4c3sc3cc5c(ccc6ccccc65)cc34)cc2)cc1 ; c1ccc(N(c2ccccc2)c2cccc(N(c3ccccc3)c3cccc4c3sc3cc5c(ccc6ccccc65)cc34)c2)cc1]

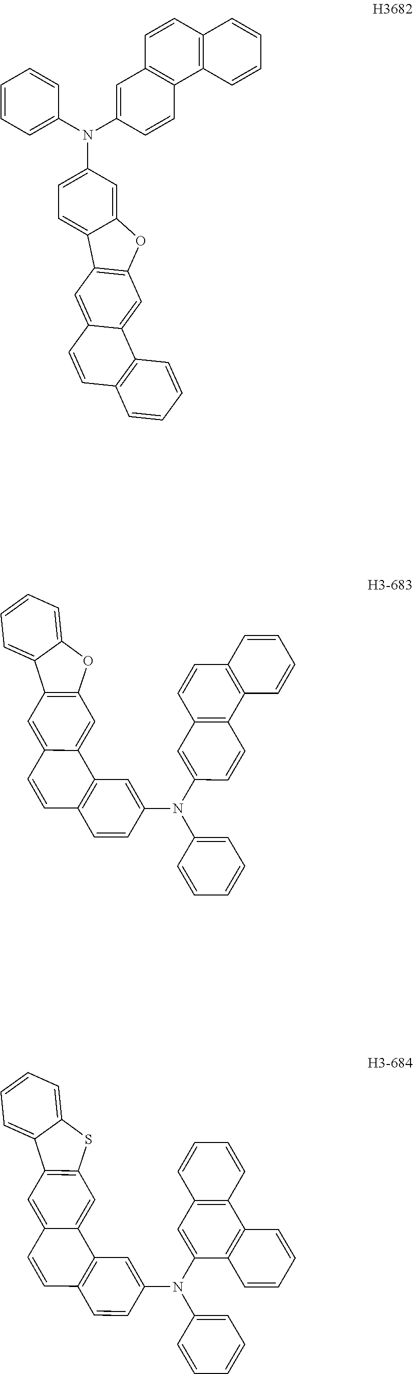 [SMILES: c1ccc(N(c2ccc3c(ccc4ccccc43)c2)c2ccc3c(c2)oc2cc4c(ccc5ccccc54)cc23)cc1 ; c1ccc(N(c2ccc3c(ccc4ccccc43)c2)c2ccc3ccc4cc5c(cc4c3c2)oc2ccccc25)cc1 ; c1ccc(N(c2ccc3ccc4cc5c(cc4c3c2)sc2ccccc25)c2cc3ccccc3c3ccccc23)cc1]

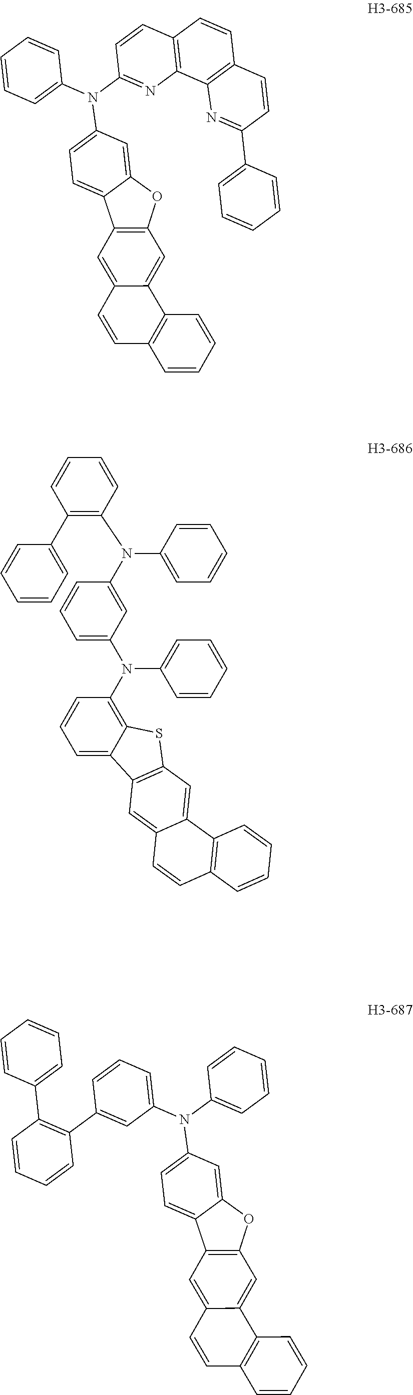 [SMILES: c1ccc(-c2ccc3ccc4ccc(N(c5ccccc5)c5ccc6c(c5)oc5cc7c(ccc8ccccc87)cc56)nc4c3n2)cc1 ; c1ccc(-c2ccccc2N(c2ccccc2)c2cccc(N(c3ccccc3)c3cccc4c3sc3cc5c(ccc6ccccc65)cc34)c2)cc1 ; c1ccc(-c2ccccc2-c2cccc(N(c3ccccc3)c3ccc4c(c3)oc3cc5c(ccc6ccccc65)cc34)c2)cc1]

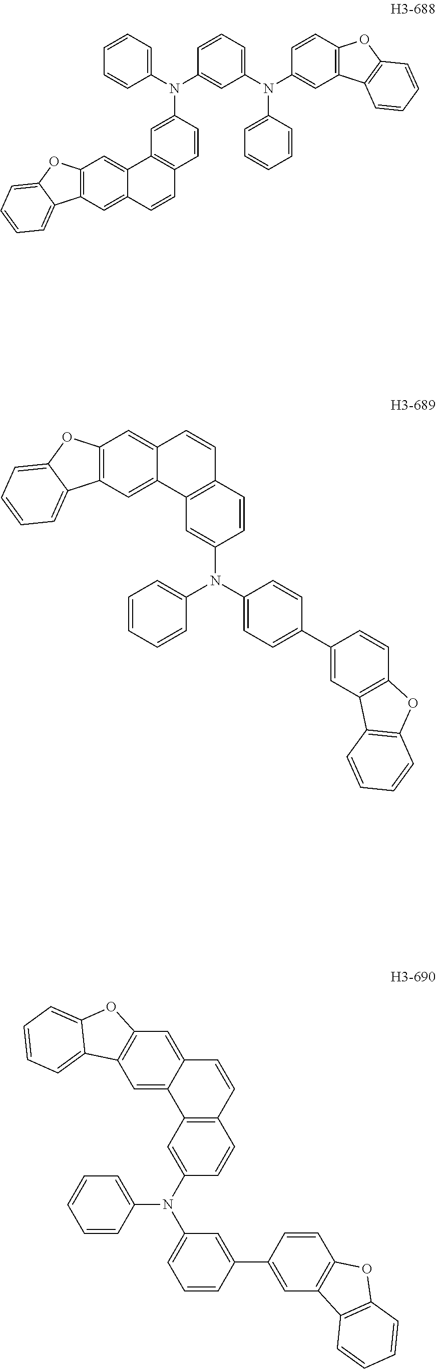 [SMILES: c1ccc(N(c2cccc(N(c3ccccc3)c3ccc4oc5ccccc5c4c3)c2)c2ccc3ccc4cc5c(cc4c3c2)oc2ccccc25)cc1 ; c1ccc(N(c2ccc(-c3ccc4oc5ccccc5c4c3)cc2)c2ccc3ccc4cc5oc6ccccc6c5cc4c3c2)cc1 ; c1ccc(N(c2cccc(-c3ccc4oc5ccccc5c4c3)c2)c2ccc3ccc4cc5oc6ccccc6c5cc4c3c2)cc1]

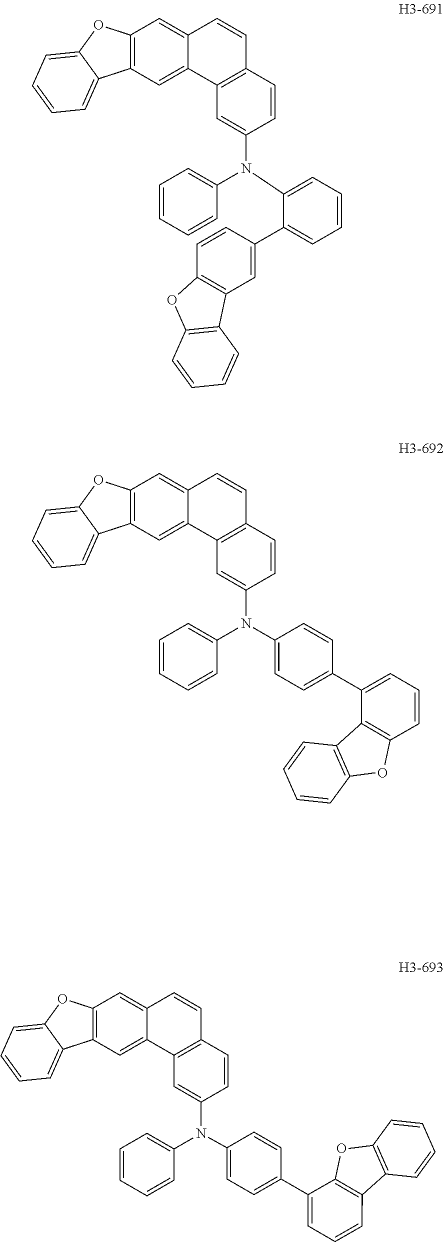 [SMILES: c1ccc(N(c2ccc3ccc4cc5oc6ccccc6c5cc4c3c2)c2ccccc2-c2ccc3oc4ccccc4c3c2)cc1 ; c1ccc(N(c2ccc(-c3cccc4oc5ccccc5c34)cc2)c2ccc3ccc4cc5oc6ccccc6c5cc4c3c2)cc1 ; c1ccc(N(c2ccc(-c3cccc4c3oc3ccccc34)cc2)c2ccc3ccc4cc5oc6ccccc6c5cc4c3c2)cc1]

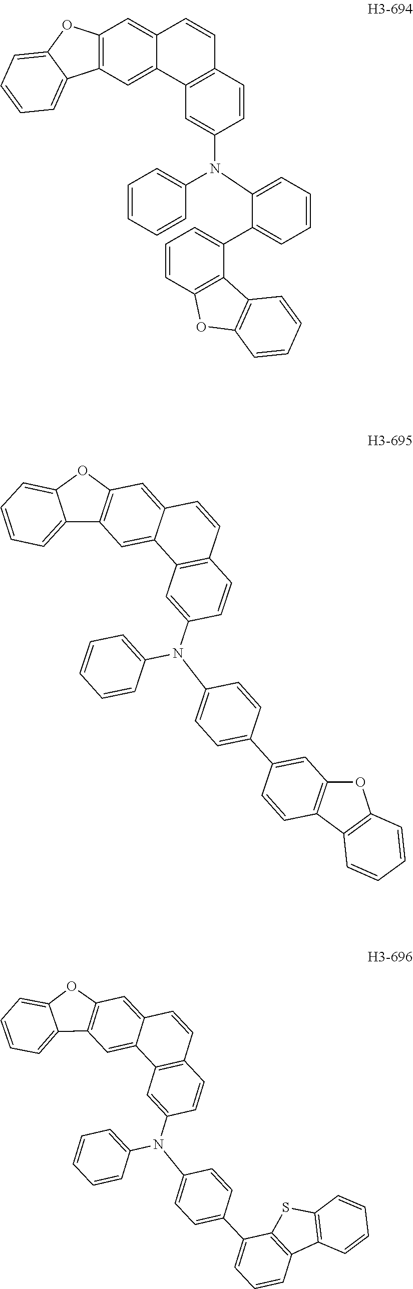 [SMILES: c1ccc(N(c2ccc3ccc4cc5oc6ccccc6c5cc4c3c2)c2ccccc2-c2cccc3oc4ccccc4c23)cc1 ; c1ccc(N(c2ccc(-c3ccc4c(c3)oc3ccccc34)cc2)c2ccc3ccc4cc5oc6ccccc6c5cc4c3c2)cc1 ; c1ccc(N(c2ccc(-c3cccc4c3sc3ccccc34)cc2)c2ccc3ccc4cc5oc6ccccc6c5cc4c3c2)cc1]

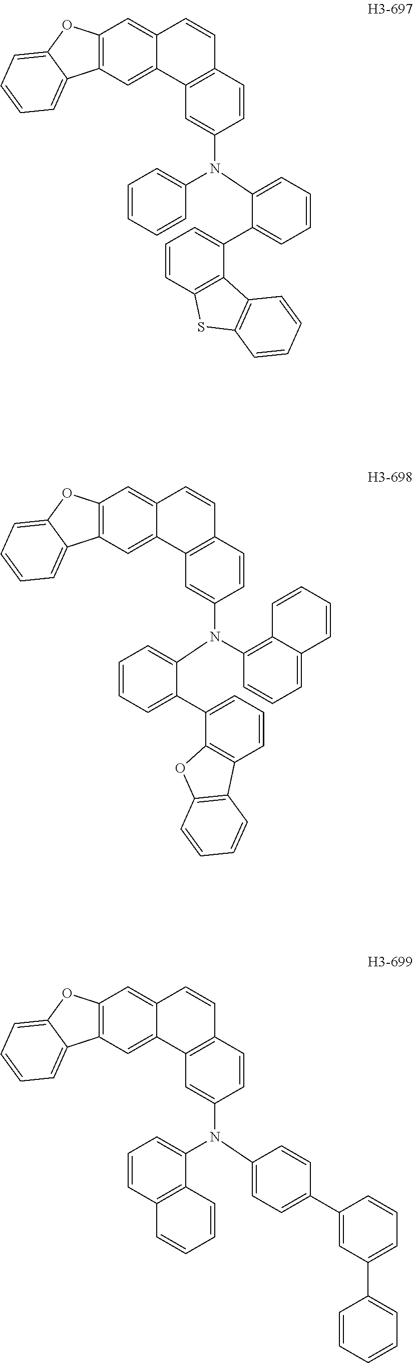

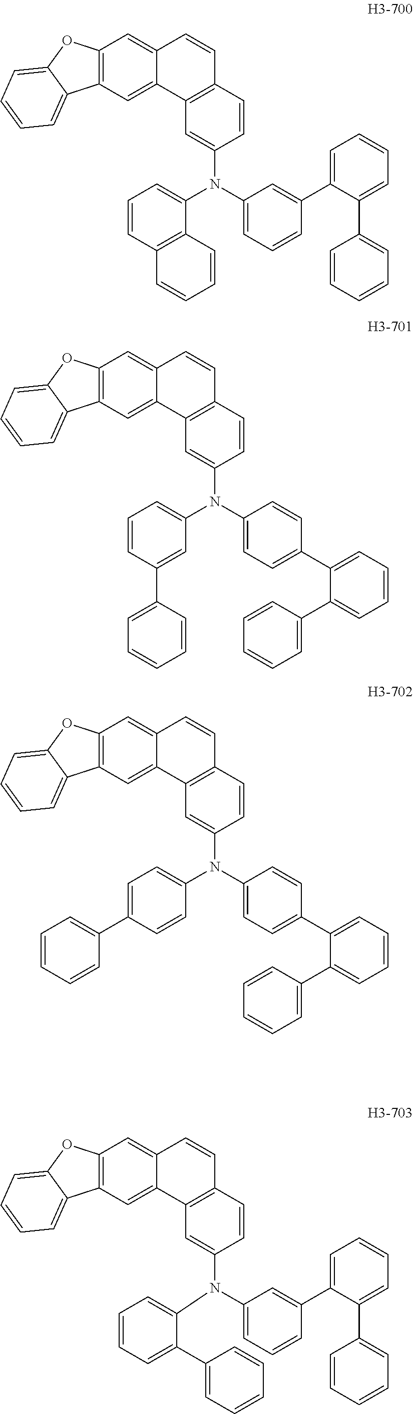

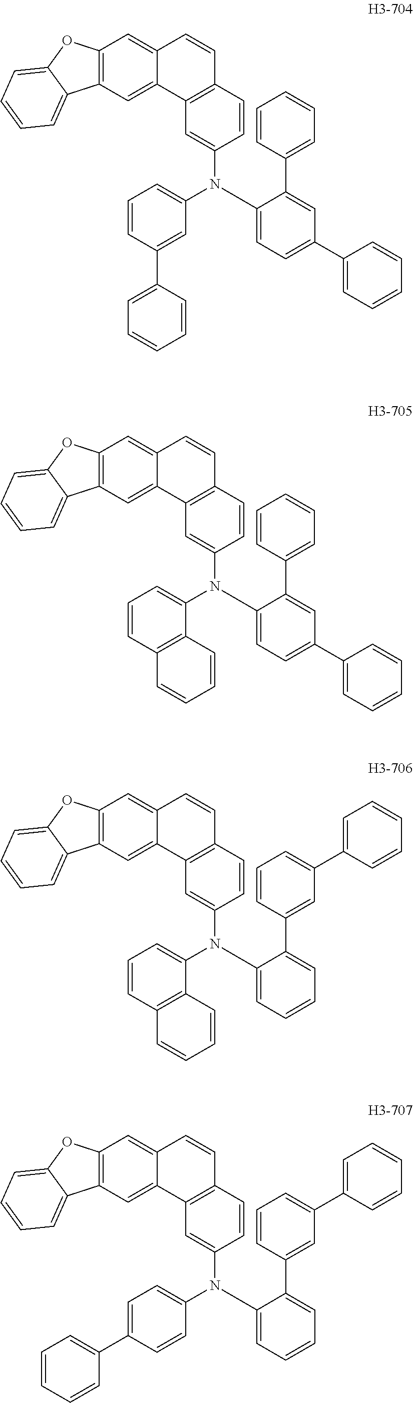 [SMILES: c1ccc(-c2cccc(N(c3ccc4ccc5cc6oc7ccccc7c6cc5c4c3)c3ccc(-c4ccccc4)cc3-c3ccccc3)c2)cc1 ; c1ccc(-c2ccc(N(c3ccc4ccc5cc6oc7ccccc7c6cc5c4c3)c3cccc4ccccc34)c(-c3ccccc3)c2)cc1 ; c1ccc(-c2cccc(-c3ccccc3N(c3ccc4ccc5cc6oc7ccccc7c6cc5c4c3)c3cccc4ccccc34)c2)cc1 ; c1ccc(-c2ccc(N(c3ccc4ccc5cc6oc7ccccc7c6cc5c4c3)c3ccccc3-c3cccc(-c4ccccc4)c3)cc2)cc1]

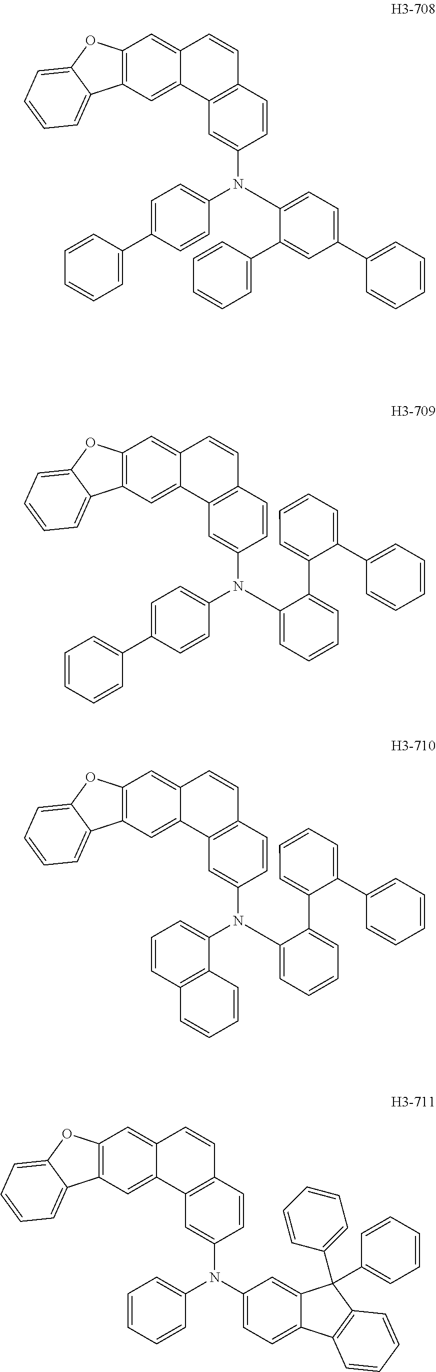 [SMILES: c1ccc(-c2ccc(N(c3ccc4ccc5cc6oc7ccccc7c6cc5c4c3)c3ccc(-c4ccccc4)cc3-c3ccccc3)cc2)cc1 ; c1ccc(-c2ccc(N(c3ccc4ccc5cc6oc7ccccc7c6cc5c4c3)c3ccccc3-c3ccccc3-c3ccccc3)cc2)cc1 ; c1ccc(-c2ccccc2-c2ccccc2N(c2ccc3ccc4cc5oc6ccccc6c5cc4c3c2)c2cccc3ccccc23)cc1 ; c1ccc(N(c2ccc3c(c2)C(c2ccccc2)(c2ccccc2)c2ccccc2-3)c2ccc3ccc4cc5oc6ccccc6c5cc4c3c2)cc1]

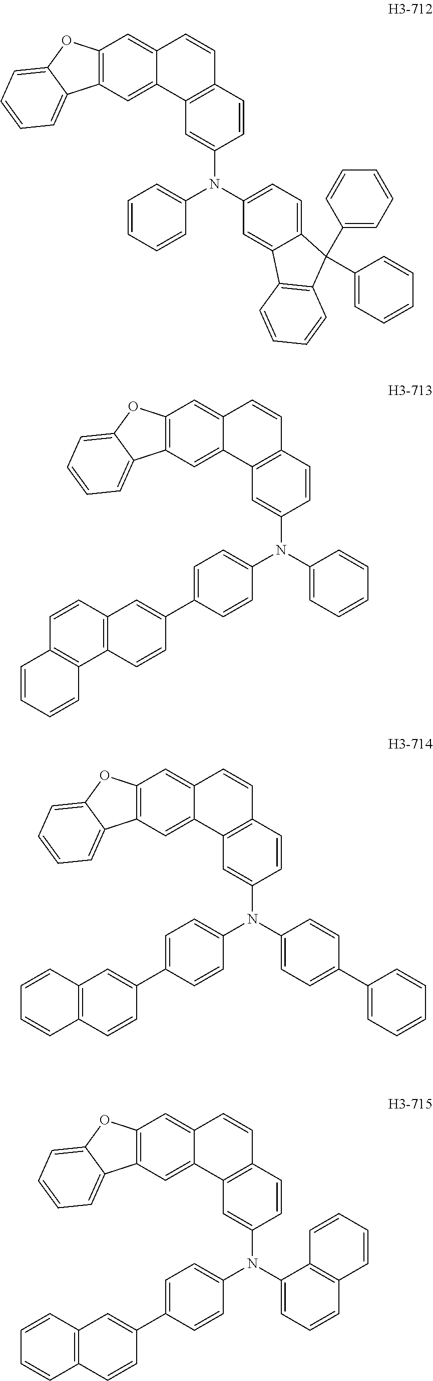 [SMILES: c1ccc(N(c2ccc3c(c2)-c2ccccc2C3(c2ccccc2)c2ccccc2)c2ccc3ccc4cc5oc6ccccc6c5cc4c3c2)cc1 ; c1ccc(N(c2ccc(-c3ccc4c(ccc5ccccc54)c3)cc2)c2ccc3ccc4cc5oc6ccccc6c5cc4c3c2)cc1 ; c1ccc(-c2ccc(N(c3ccc(-c4ccc5ccccc5c4)cc3)c3ccc4ccc5cc6oc7ccccc7c6cc5c4c3)cc2)cc1 ; c1ccc2cc(-c3ccc(N(c4ccc5ccc6cc7oc8ccccc8c7cc6c5c4)c4cccc5ccccc45)cc3)ccc2c1]

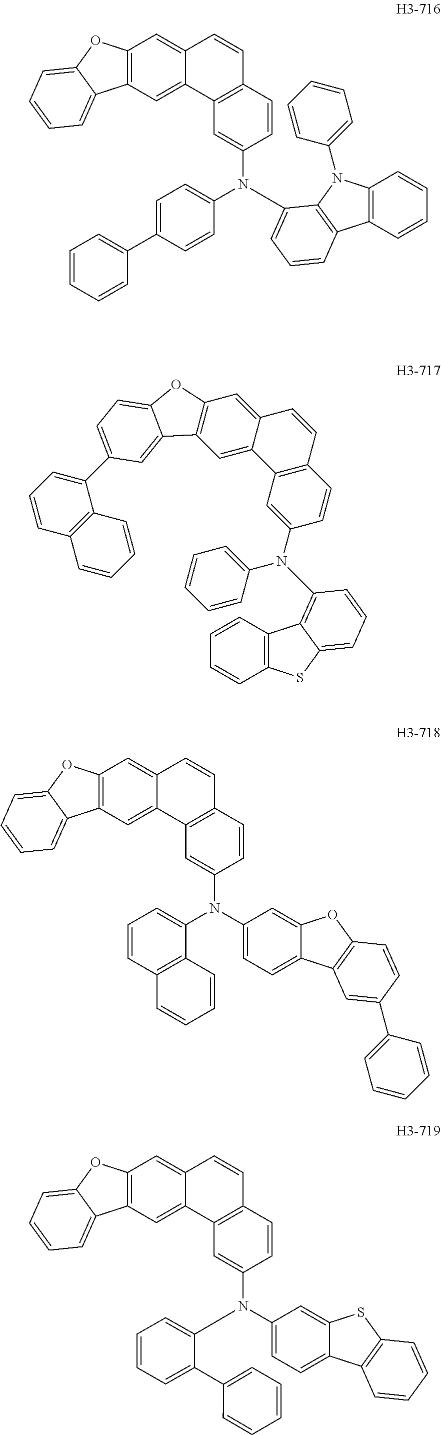 [SMILES: c1ccc(-c2ccc(N(c3ccc4ccc5cc6oc7ccccc7c6cc5c4c3)c3cccc4c5ccccc5n(-c5ccccc5)c34)cc2)cc1 ; c1ccc(N(c2ccc3ccc4cc5oc6ccc(-c7cccc8ccccc78)cc6c5cc4c3c2)c2cccc3sc4ccccc4c23)cc1 ; c1ccc(-c2ccc3oc4cc(N(c5ccc6ccc7cc8oc9ccccc9c8cc7c6c5)c5cccc6ccccc56)ccc4c3c2)cc1 ; c1ccc(-c2ccccc2N(c2ccc3c(c2)sc2ccccc23)c2ccc3ccc4cc5oc6ccccc6c5cc4c3c2)cc1]

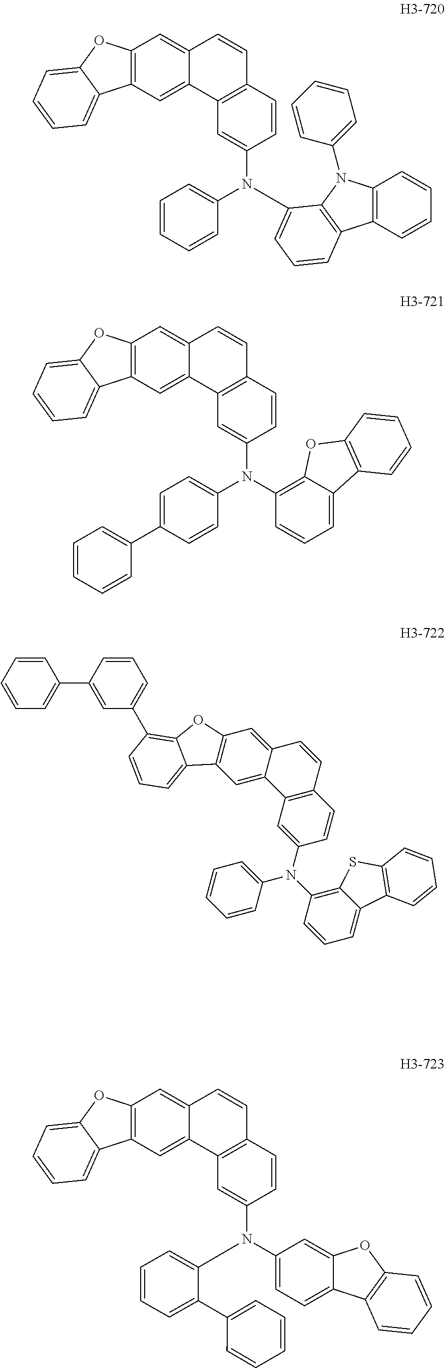 [SMILES: c1ccc(N(c2ccc3ccc4cc5oc6ccccc6c5cc4c3c2)c2cccc3c4ccccc4n(-c4ccccc4)c23)cc1 ; c1ccc(-c2ccc(N(c3ccc4ccc5cc6oc7ccccc7c6cc5c4c3)c3cccc4c3oc3ccccc34)cc2)cc1 ; c1ccc(-c2cccc(-c3cccc4c3oc3cc5ccc6ccc(N(c7ccccc7)c7cccc8c7sc7ccccc78)cc6c5cc34)c2)cc1 ; c1ccc(-c2ccccc2N(c2ccc3c(c2)oc2ccccc23)c2ccc3ccc4cc5oc6ccccc6c5cc4c3c2)cc1]

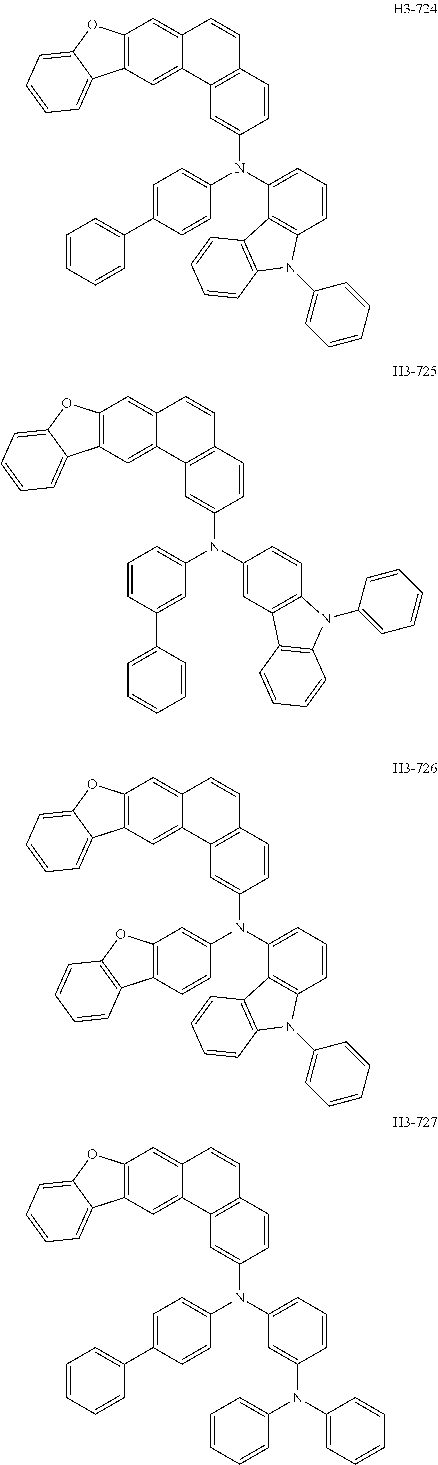 [SMILES: c1ccc(-c2ccc(N(c3ccc4ccc5cc6oc7ccccc7c6cc5c4c3)c3cccc4c3c3ccccc3n4-c3ccccc3)cc2)cc1 ; c1ccc(-c2cccc(N(c3ccc4ccc5cc6oc7ccccc7c6cc5c4c3)c3ccc4c(c3)c3ccccc3n4-c3ccccc3)c2)cc1 ; c1ccc(-n2c3ccccc3c3c(N(c4ccc5c(c4)oc4ccccc45)c4ccc5ccc6cc7oc8ccccc8c7cc6c5c4)cccc32)cc1 ; c1ccc(-c2ccc(N(c3cccc(N(c4ccccc4)c4ccccc4)c3)c3ccc4ccc5cc6oc7ccccc7c6cc5c4c3)cc2)cc1]

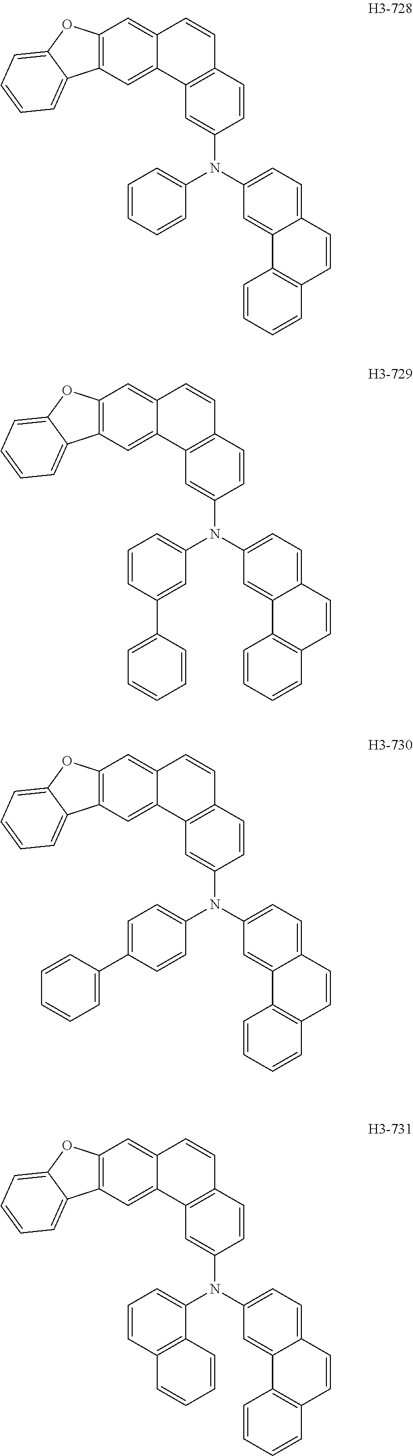 [SMILES: c1ccc(N(c2ccc3ccc4ccccc4c3c2)c2ccc3ccc4cc5oc6ccccc6c5cc4c3c2)cc1 ; c1ccc(-c2cccc(N(c3ccc4ccc5ccccc5c4c3)c3ccc4ccc5cc6oc7ccccc7c6cc5c4c3)c2)cc1 ; c1ccc(-c2ccc(N(c3ccc4ccc5ccccc5c4c3)c3ccc4ccc5cc6oc7ccccc7c6cc5c4c3)cc2)cc1 ; c1ccc2c(N(c3ccc4ccc5ccccc5c4c3)c3ccc4ccc5cc6oc7ccccc7c6cc5c4c3)cccc2c1]

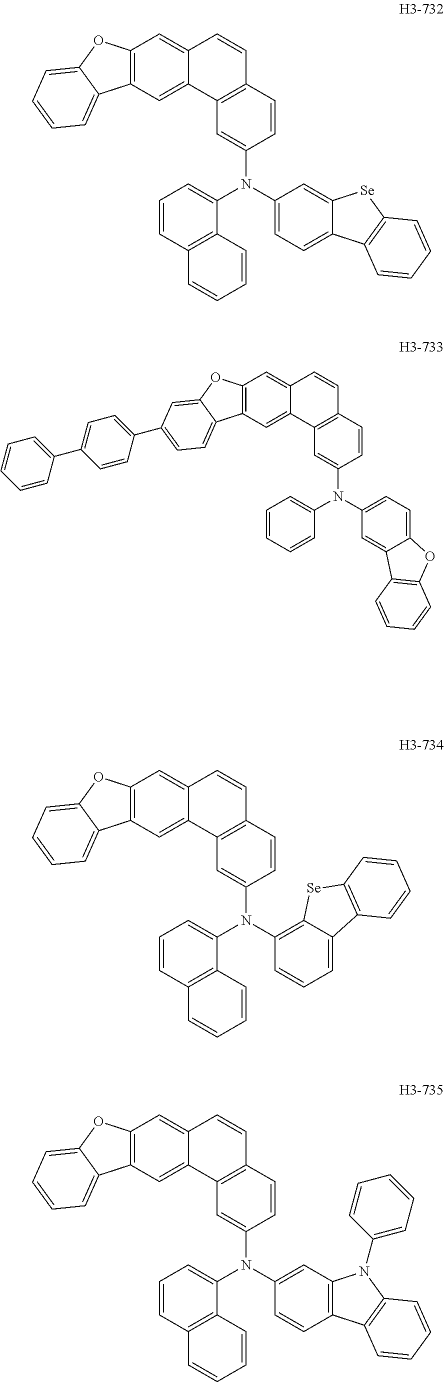 [SMILES: c1ccc2c(N(c3ccc4c(c3)[se]c3ccccc34)c3ccc4ccc5cc6oc7ccccc7c6cc5c4c3)cccc2c1 ; c1ccc(-c2ccc(-c3ccc4c(c3)oc3cc5ccc6ccc(N(c7ccccc7)c7ccc8oc9ccccc9c8c7)cc6c5cc34)cc2)cc1 ; c1ccc2c(N(c3ccc4ccc5cc6oc7ccccc7c6cc5c4c3)c3cccc4c3[se]c3ccccc34)cccc2c1 ; c1ccc(-n2c3ccccc3c3ccc(N(c4ccc5ccc6cc7oc8ccccc8c7cc6c5c4)c4cccc5ccccc45)cc32)cc1]

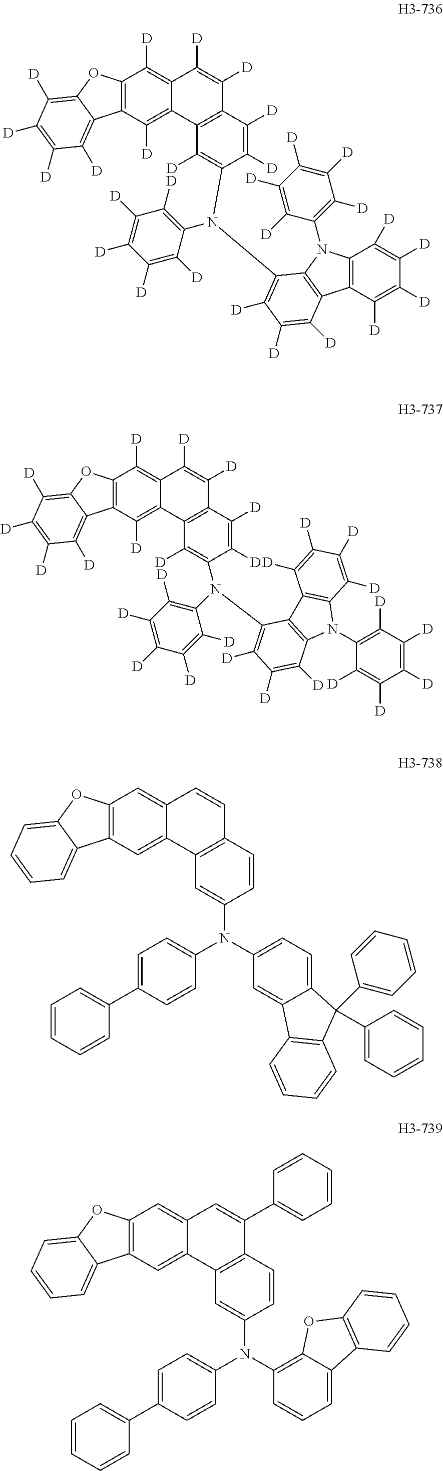

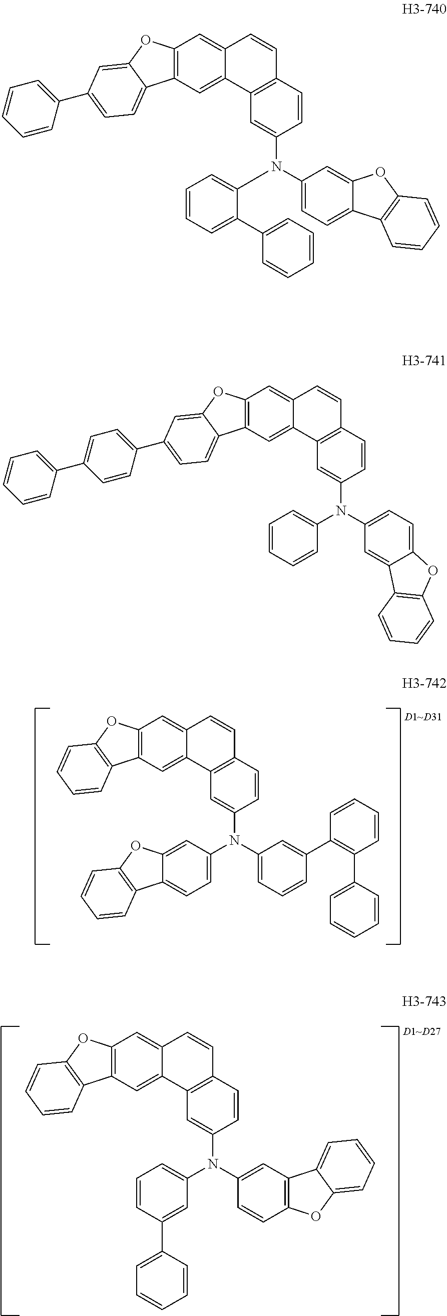

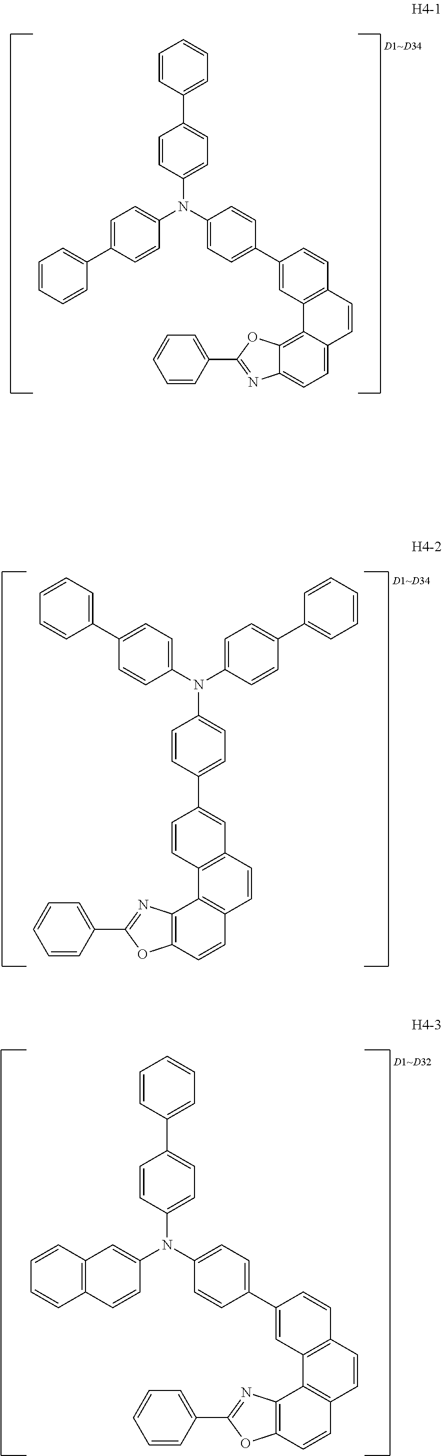

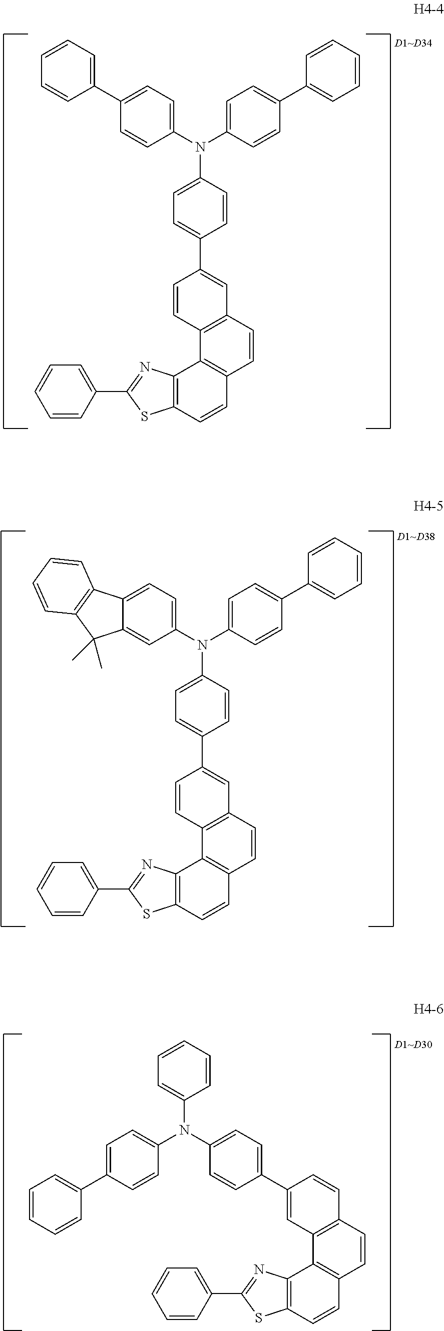

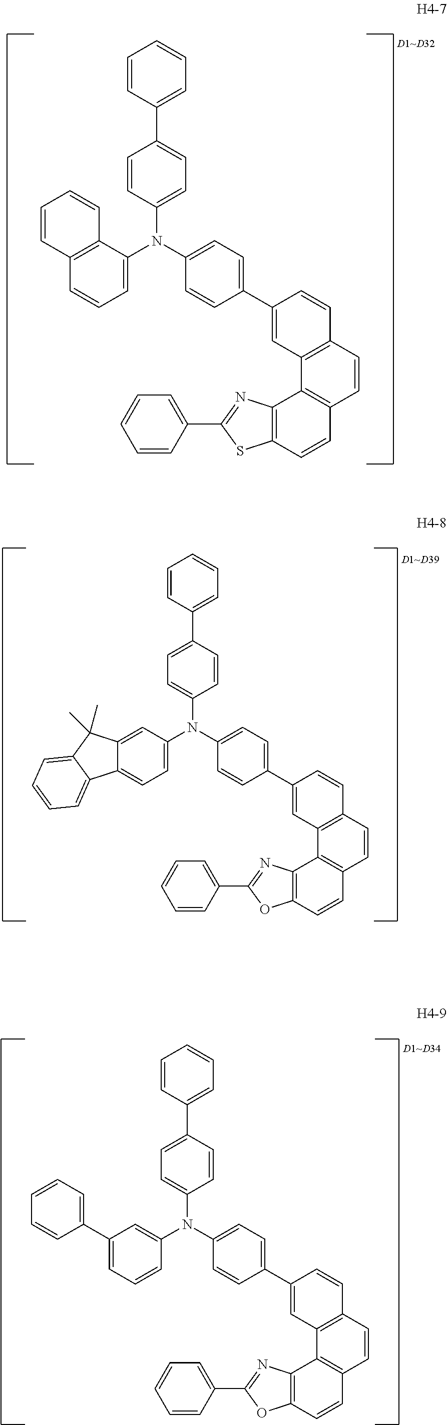

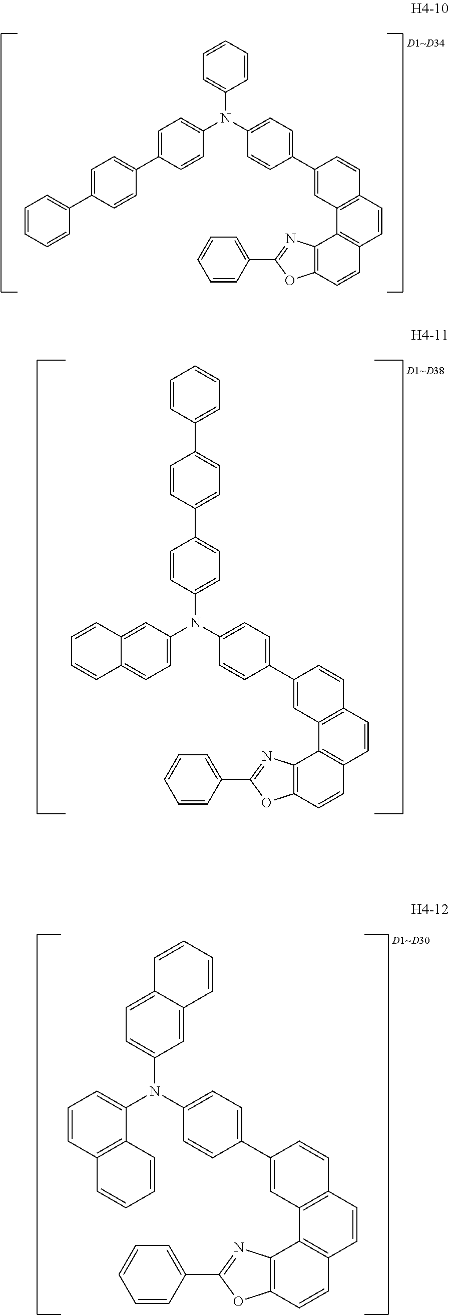

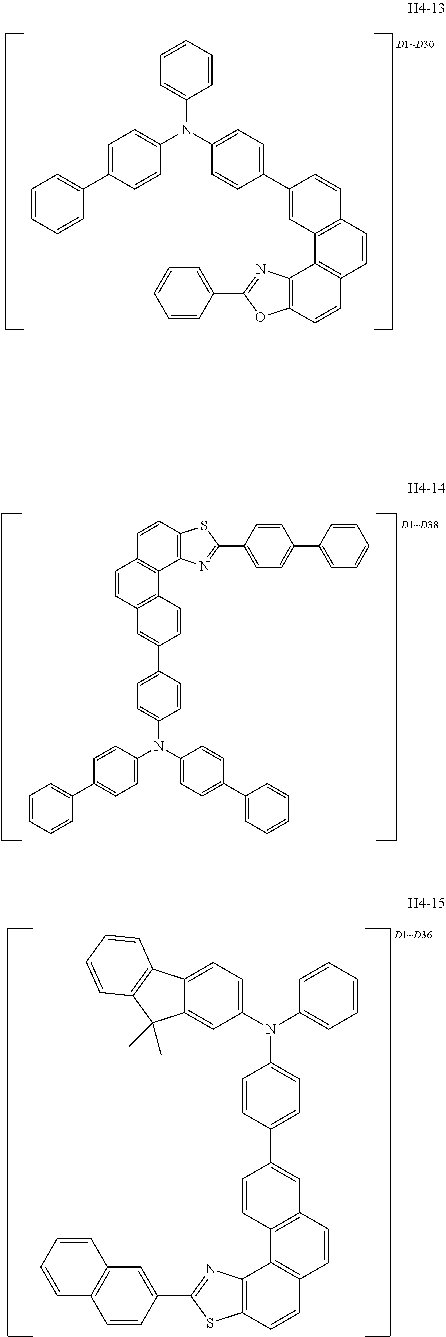

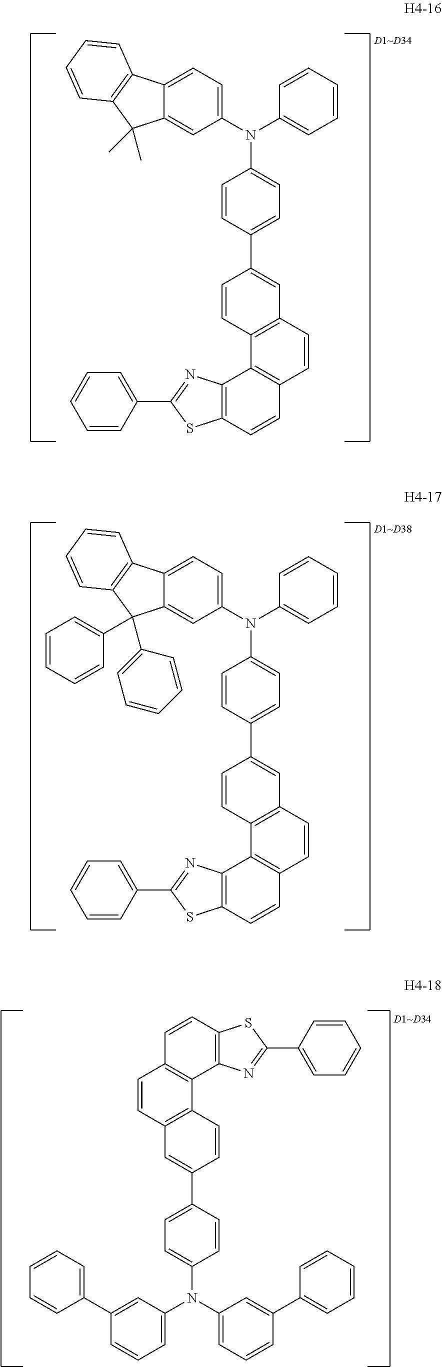

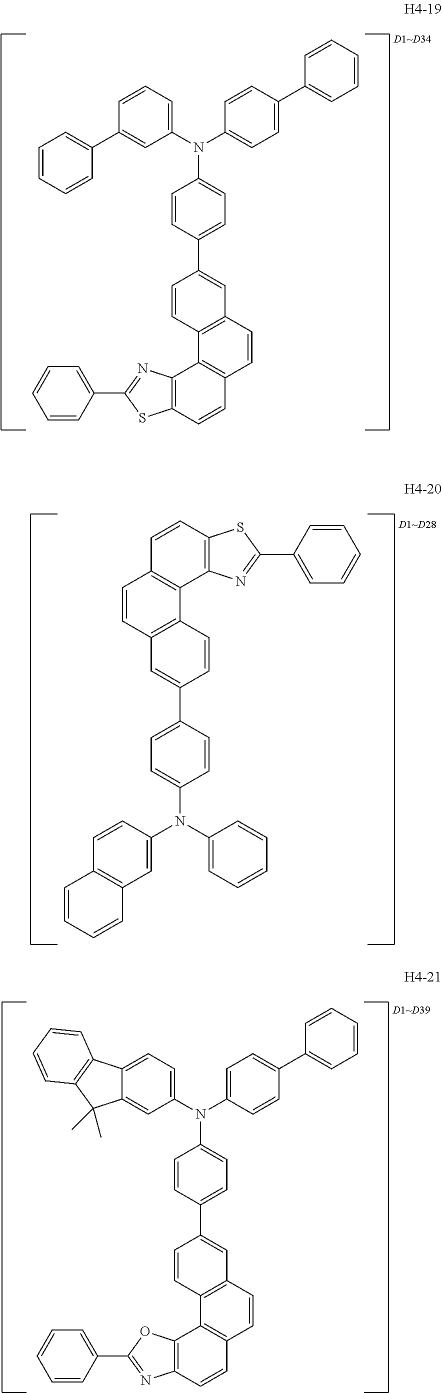

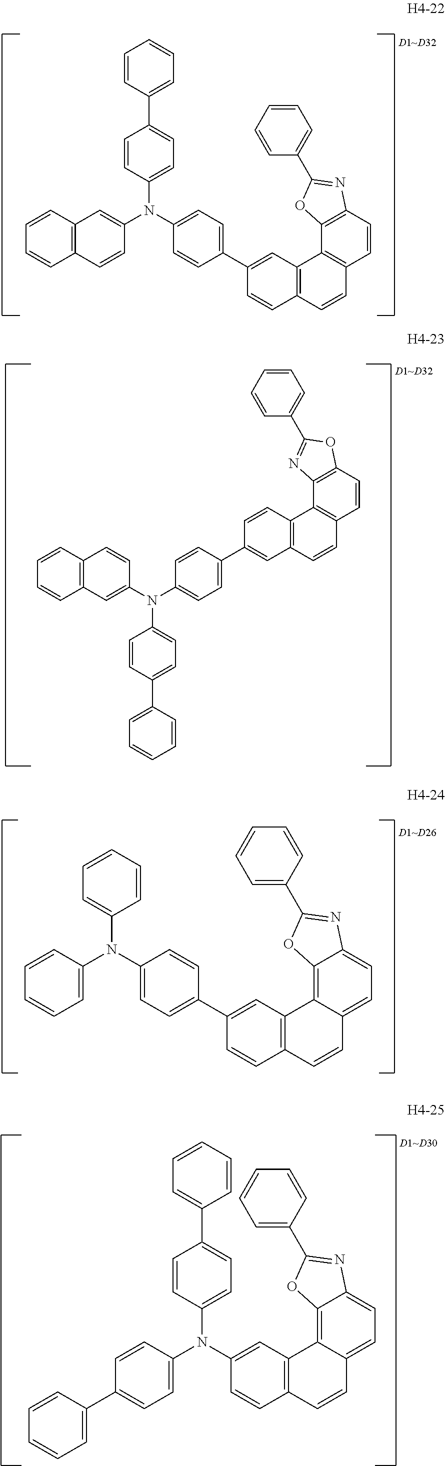

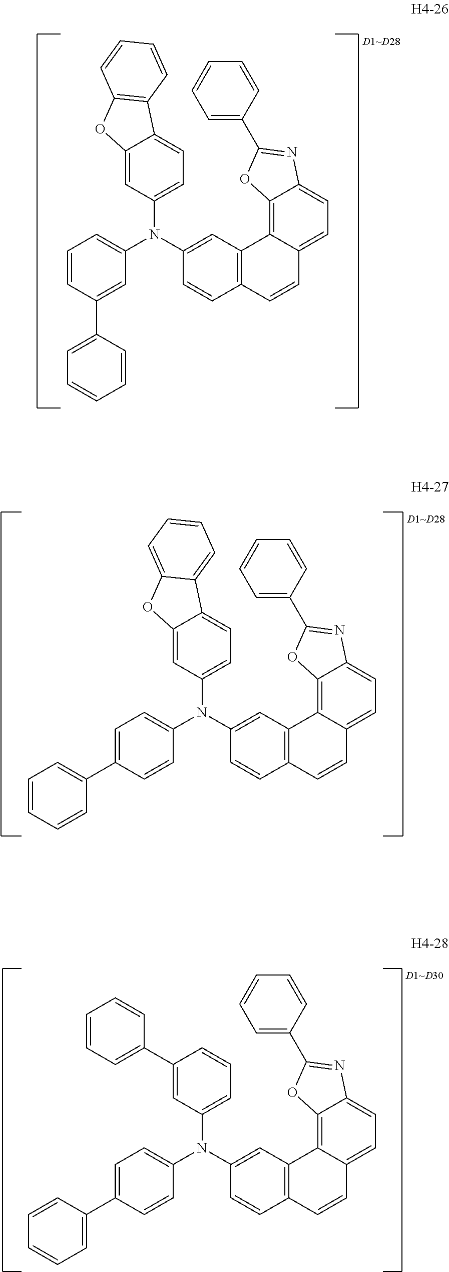

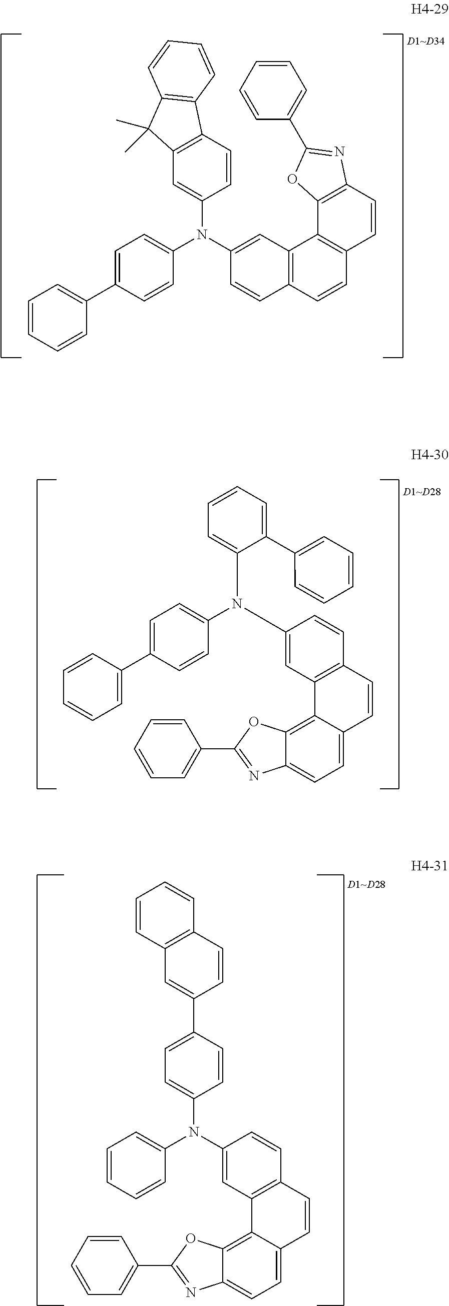

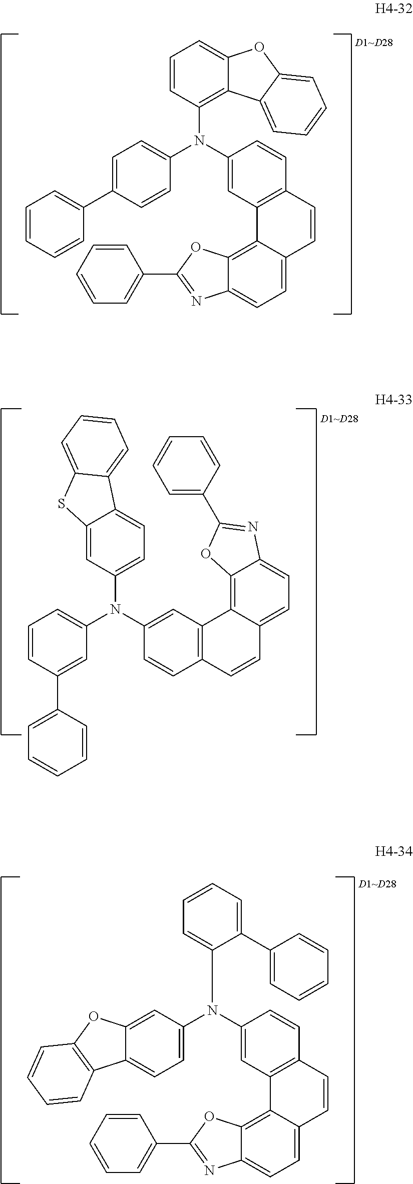

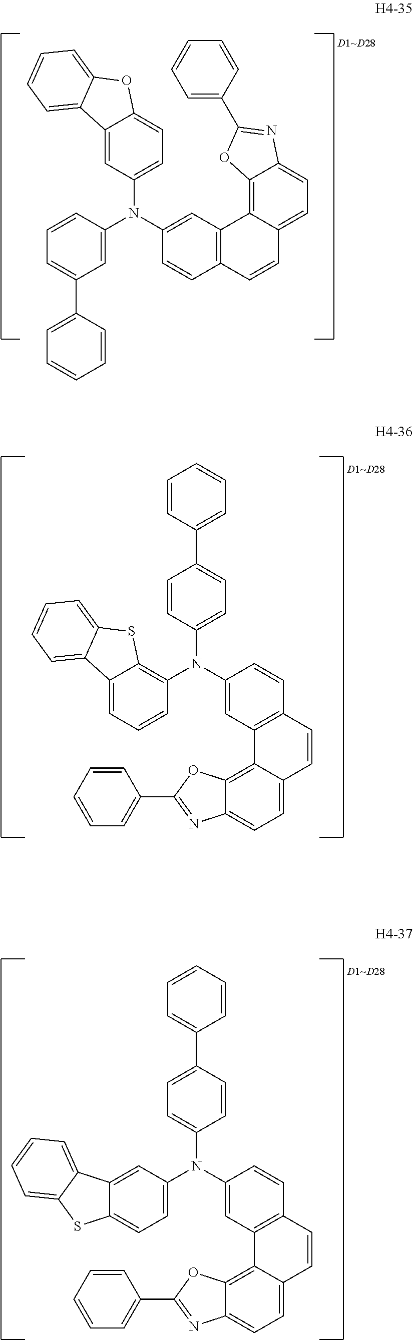

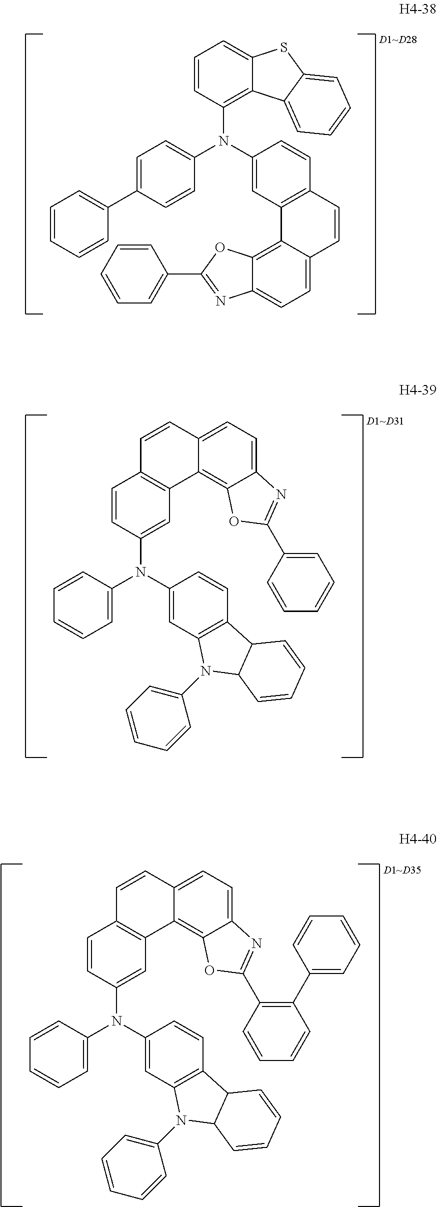

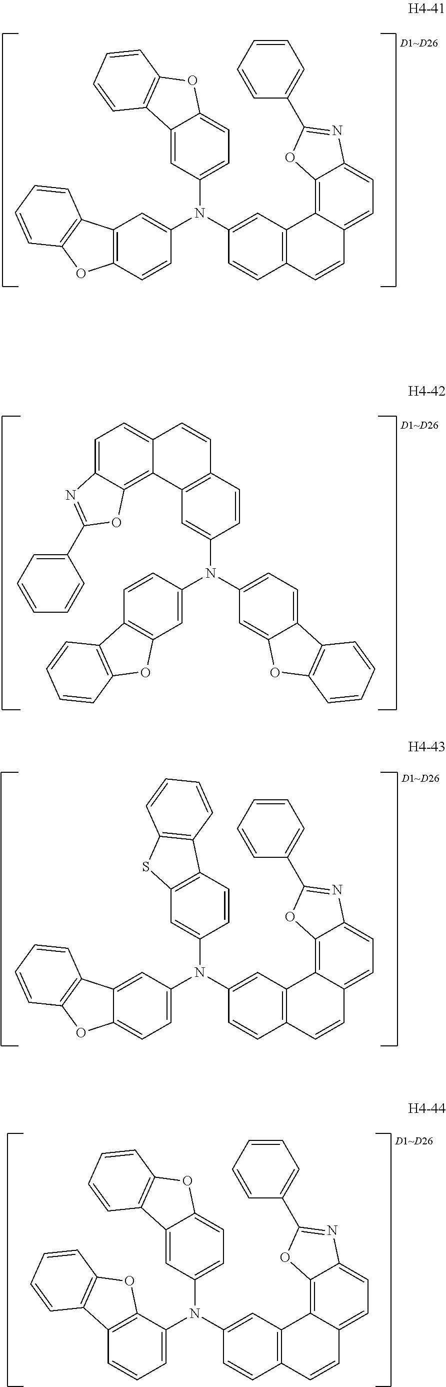

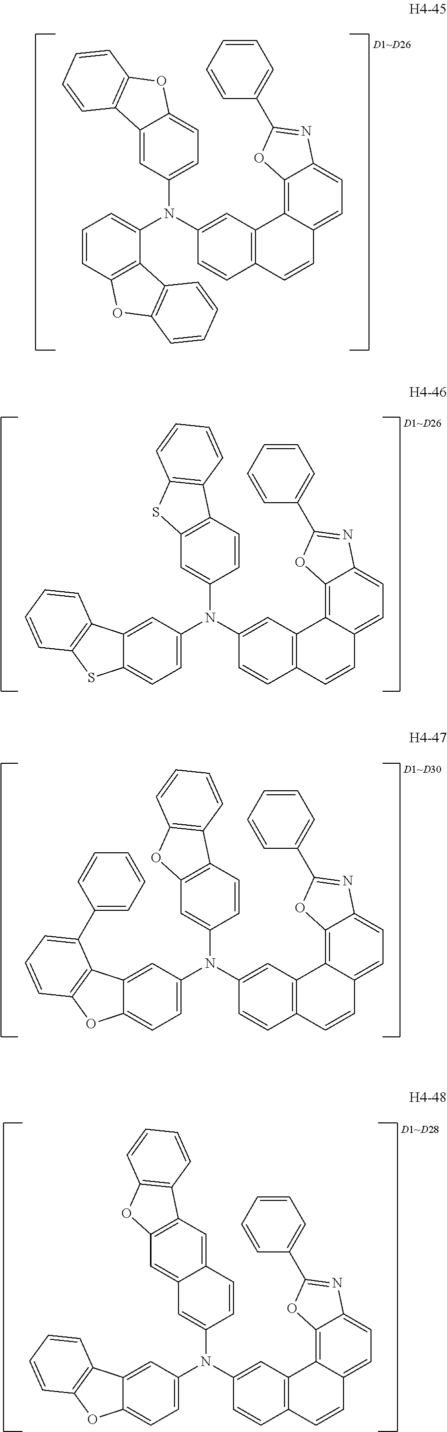

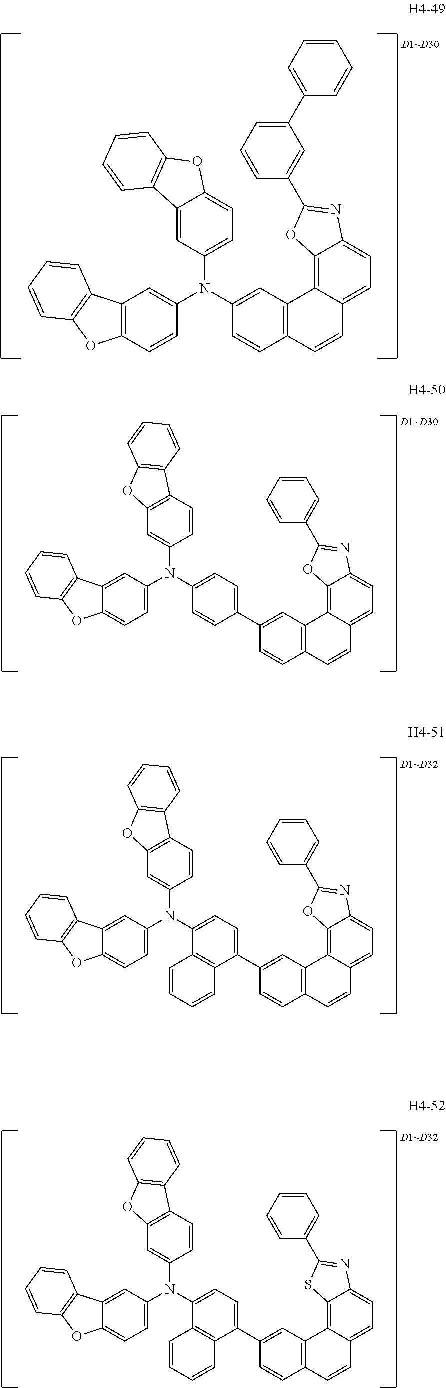

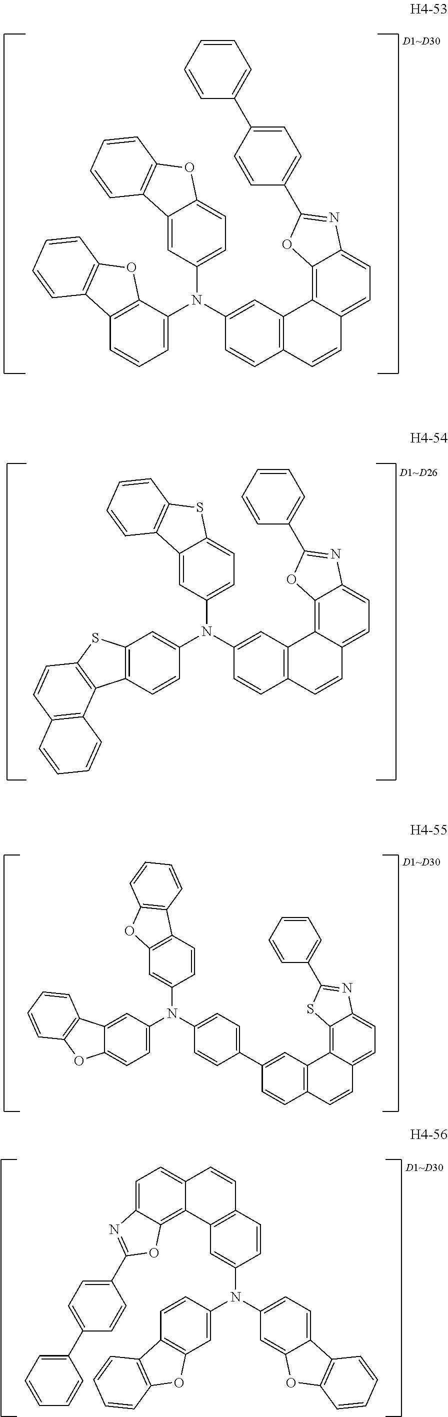

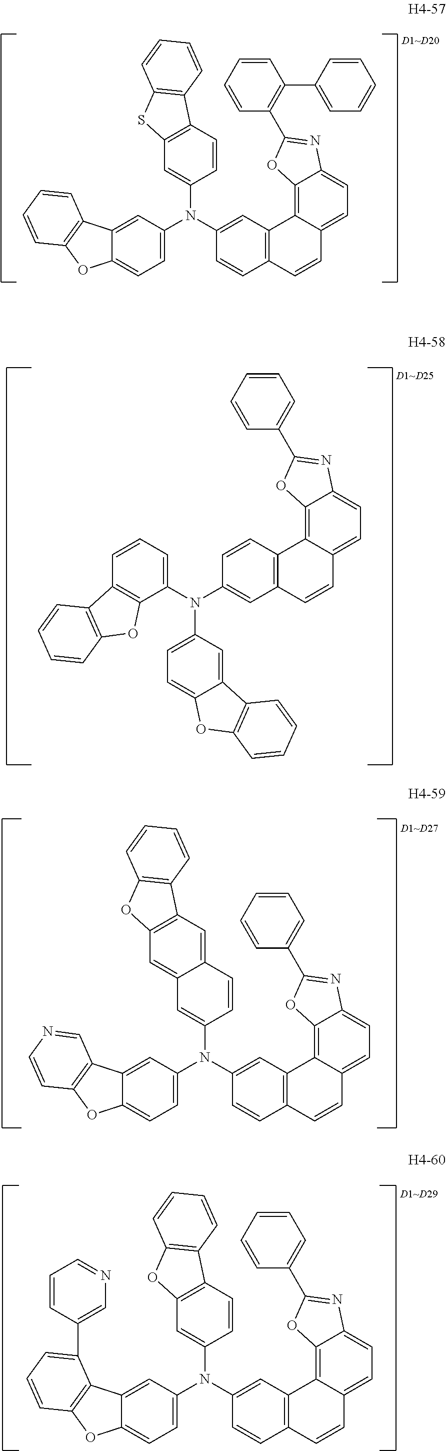

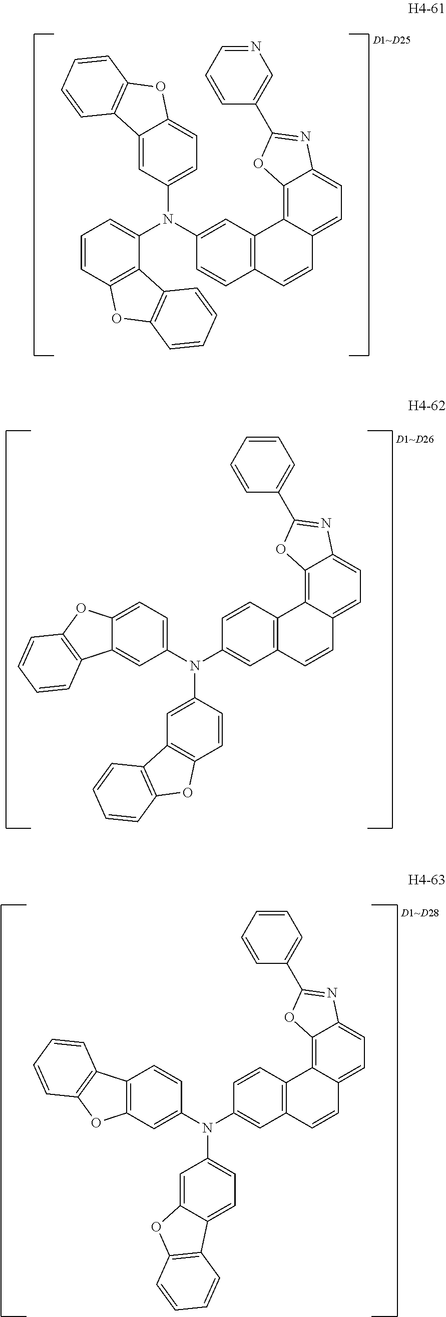

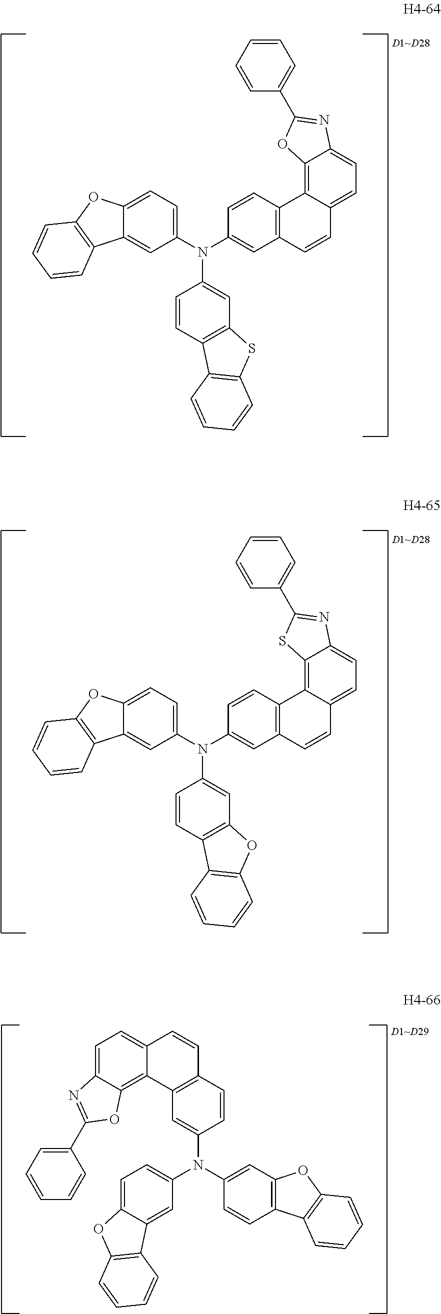

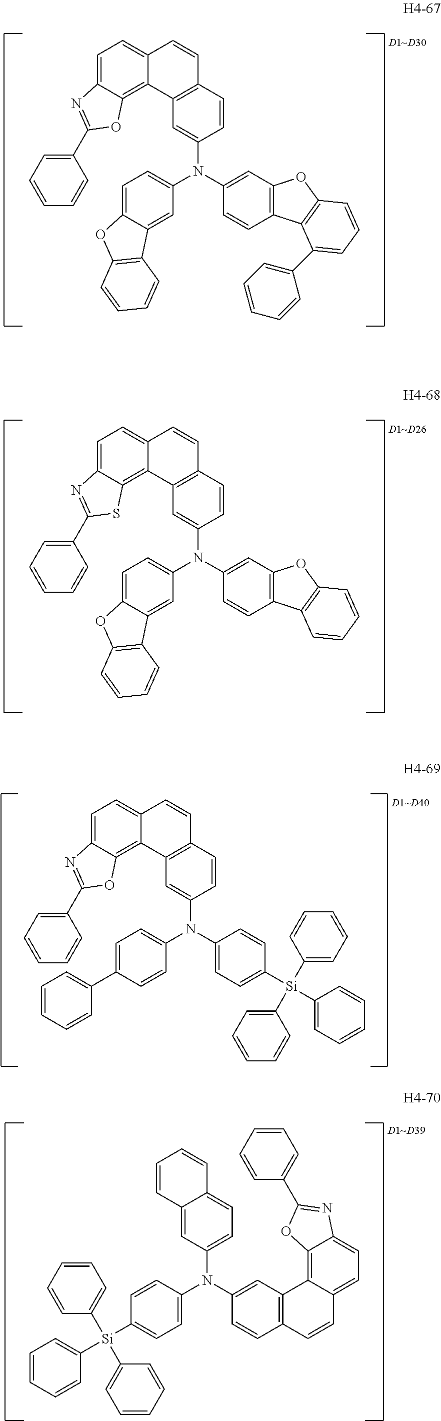

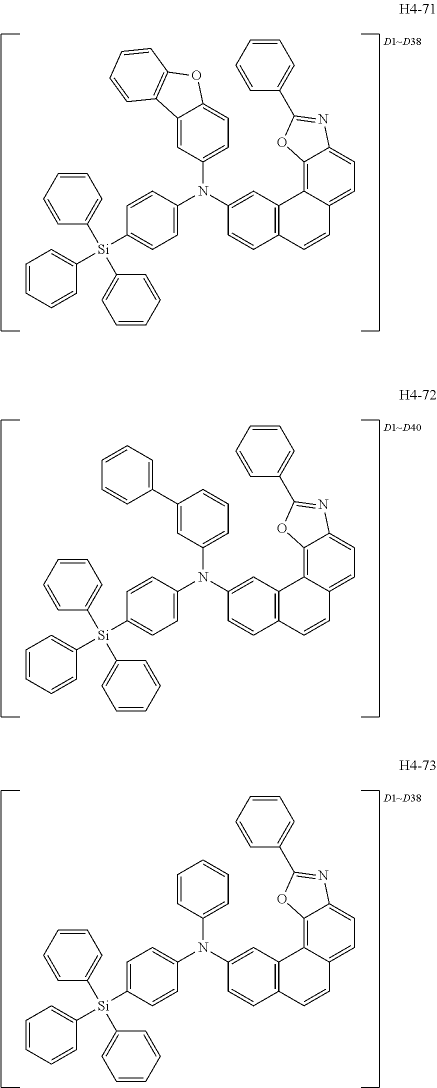

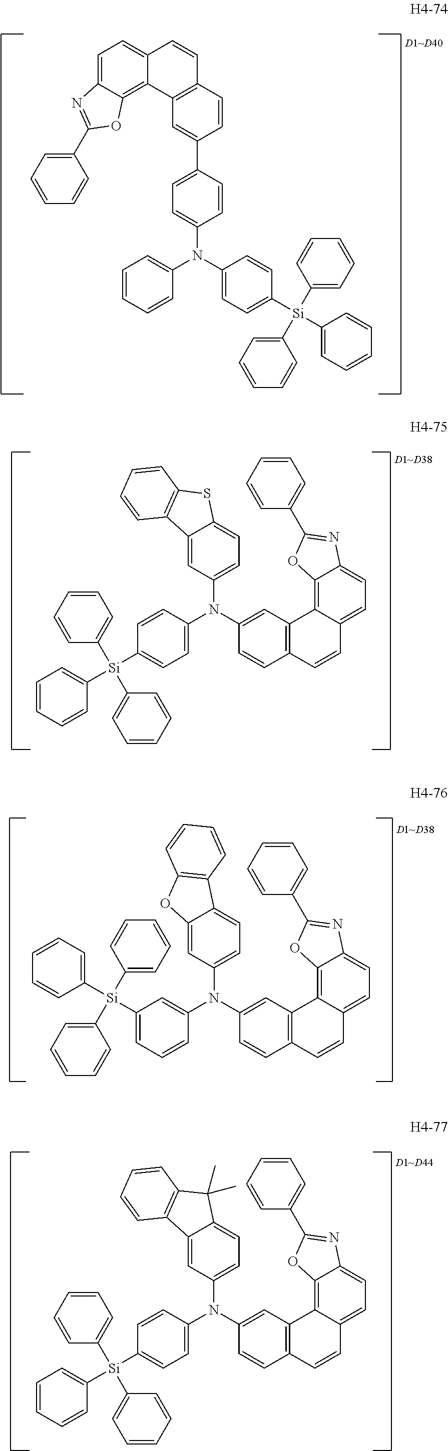

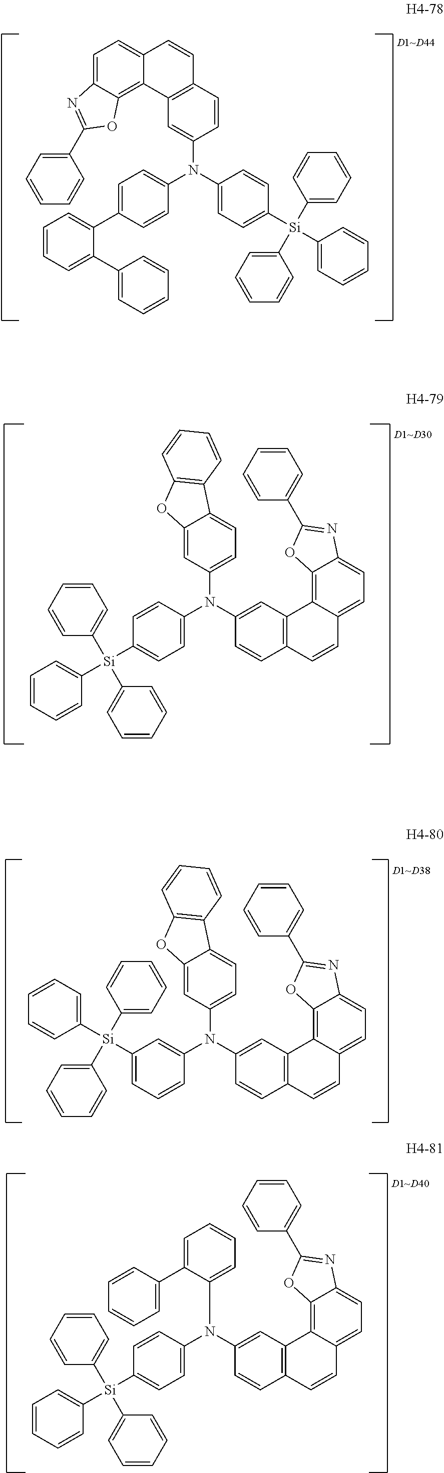

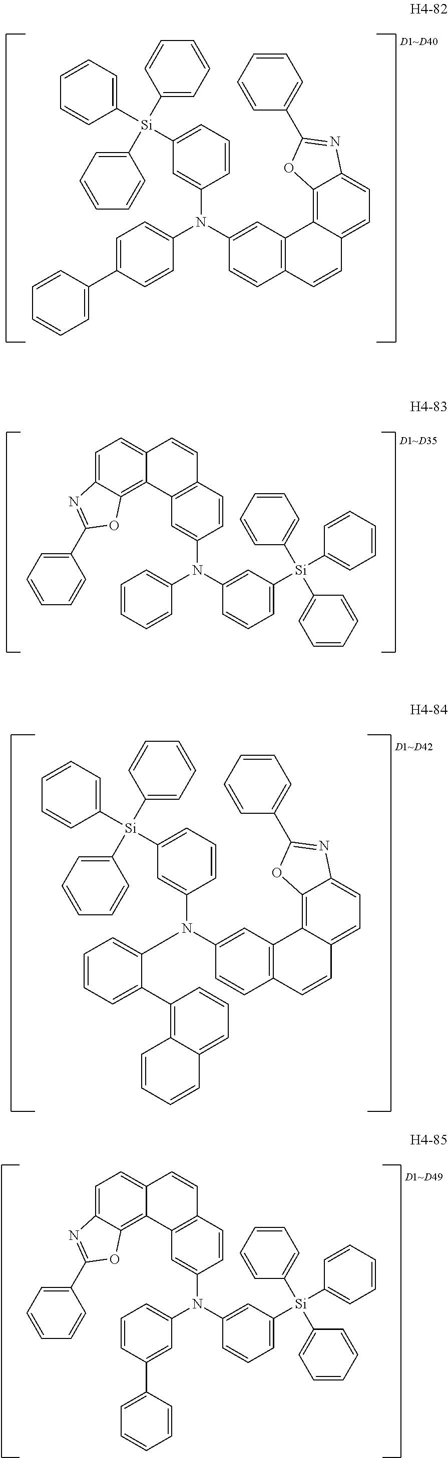

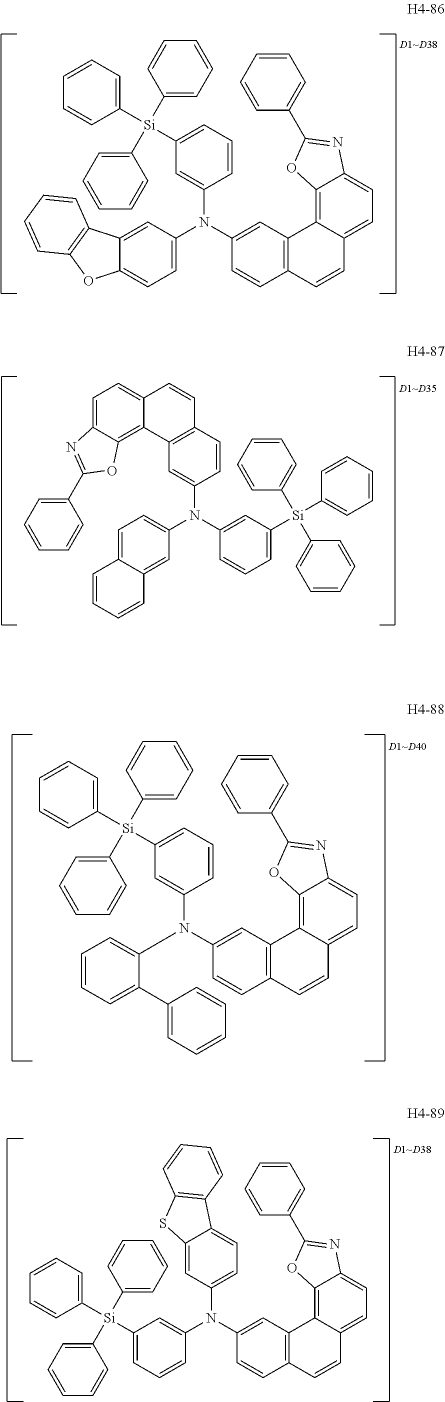

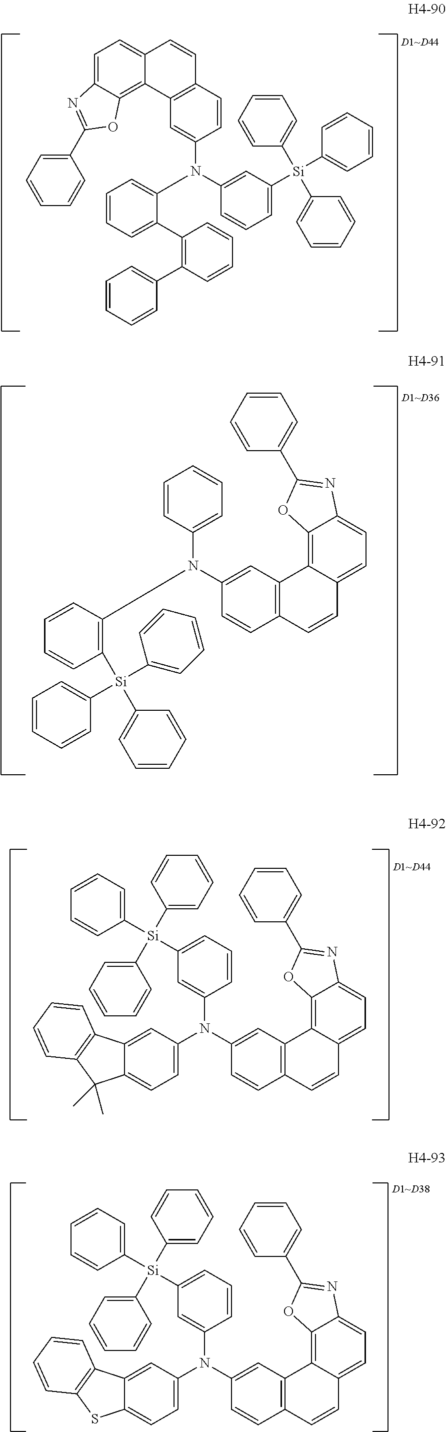

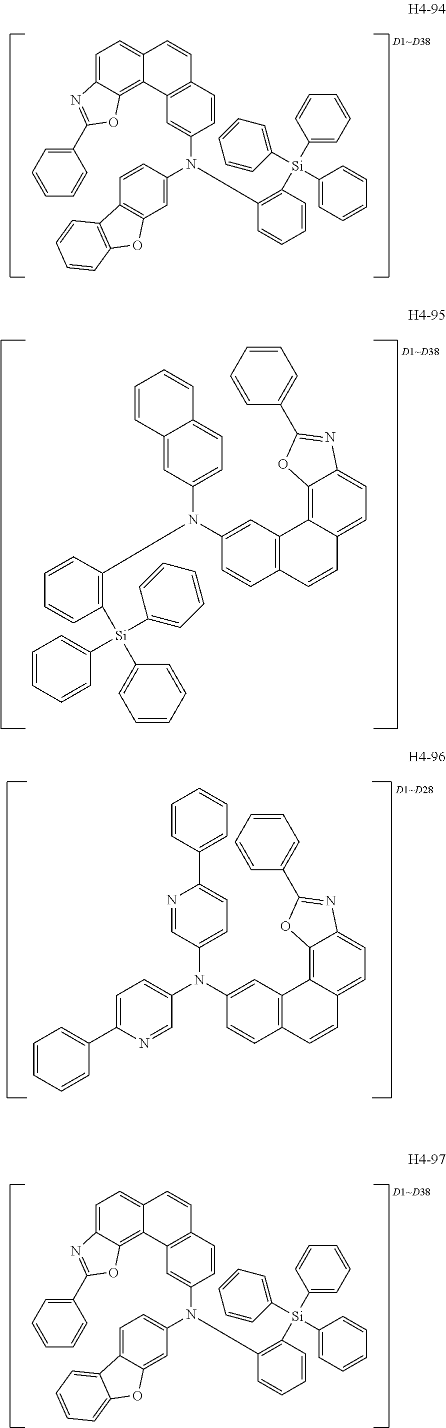

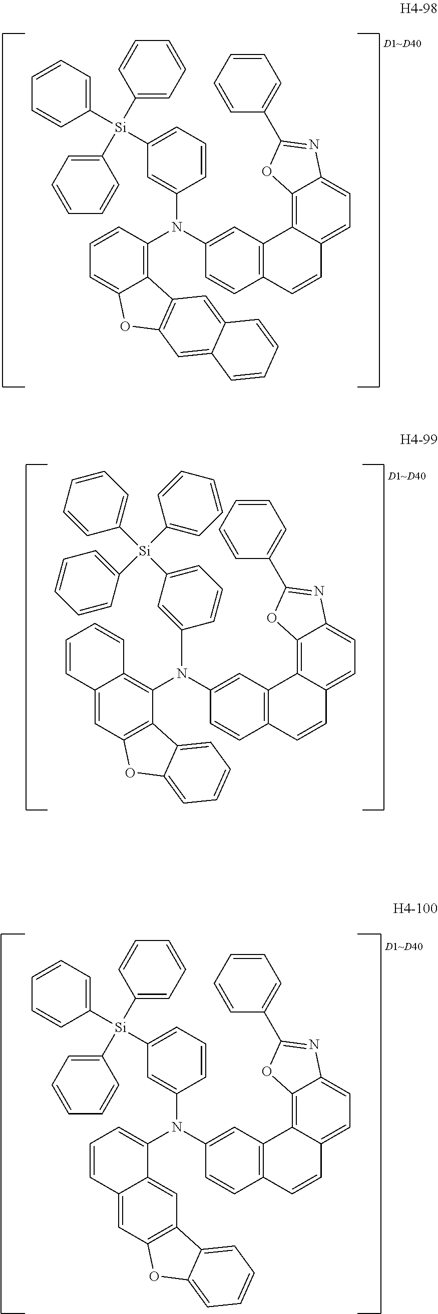

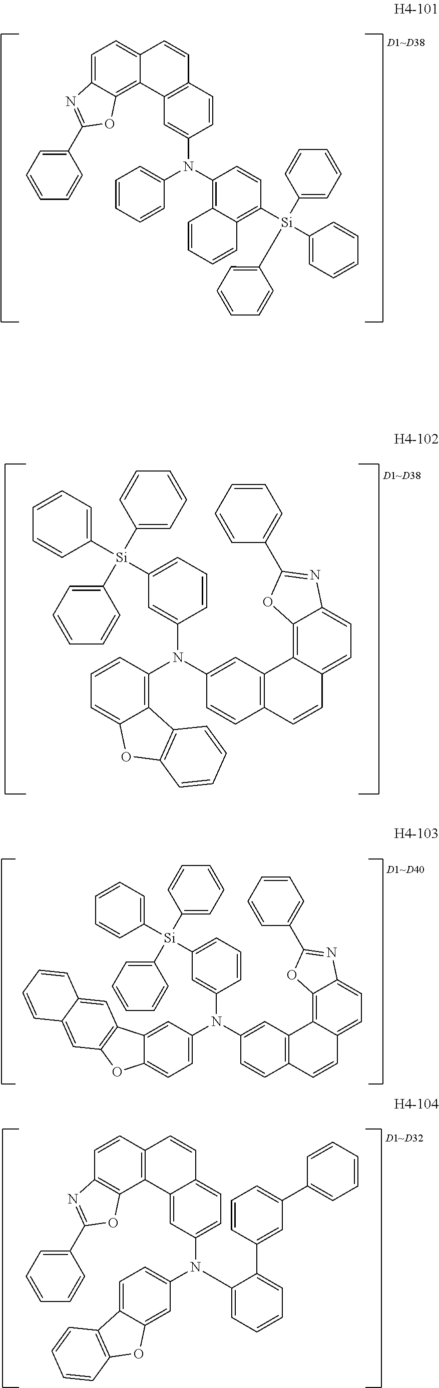

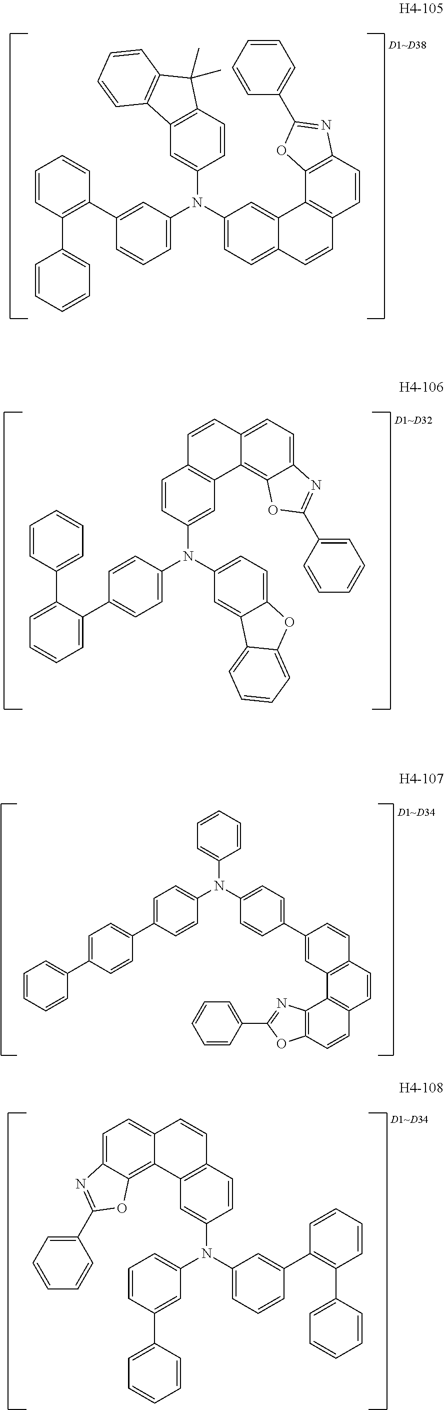

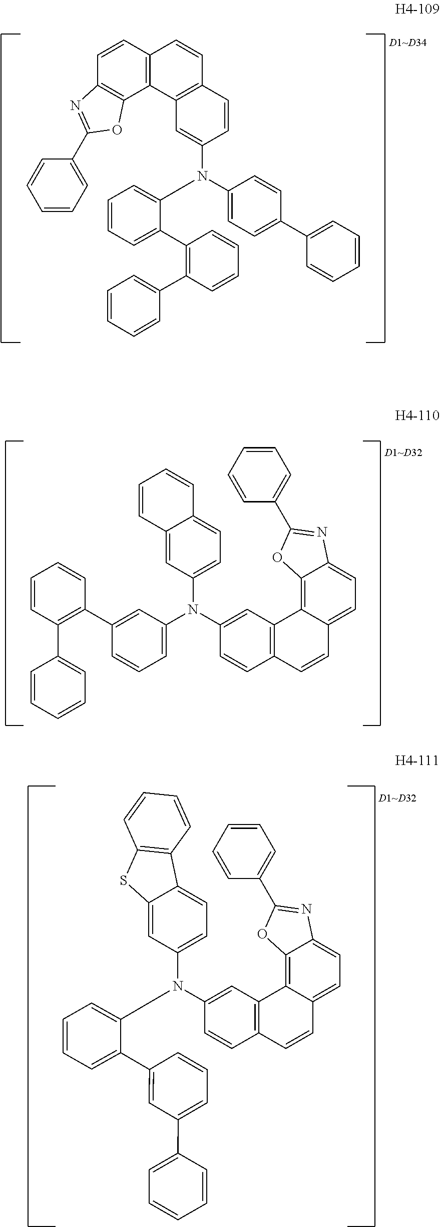

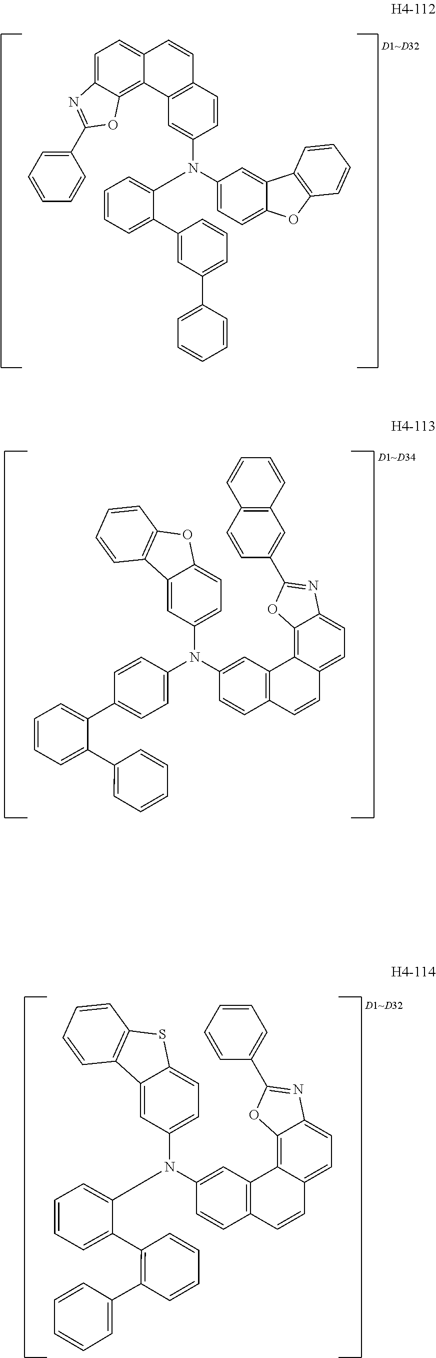

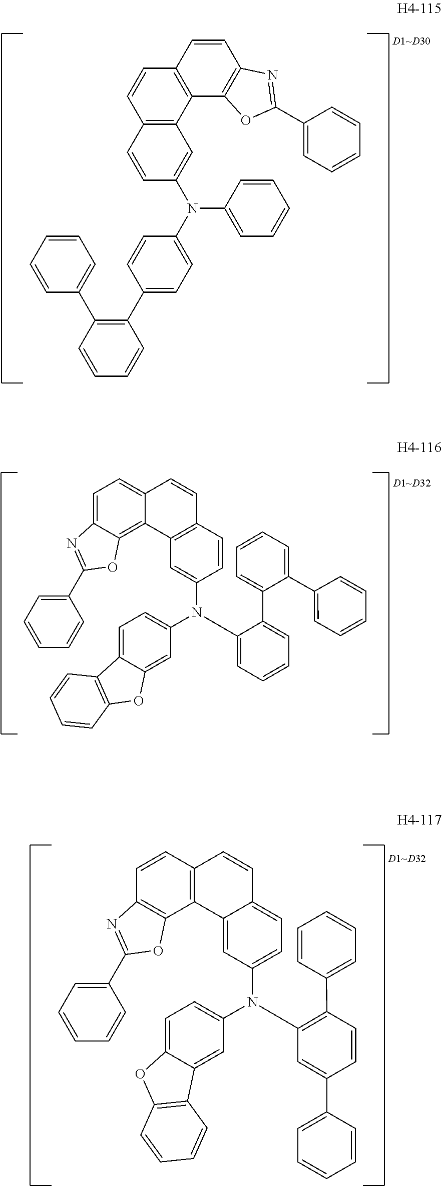

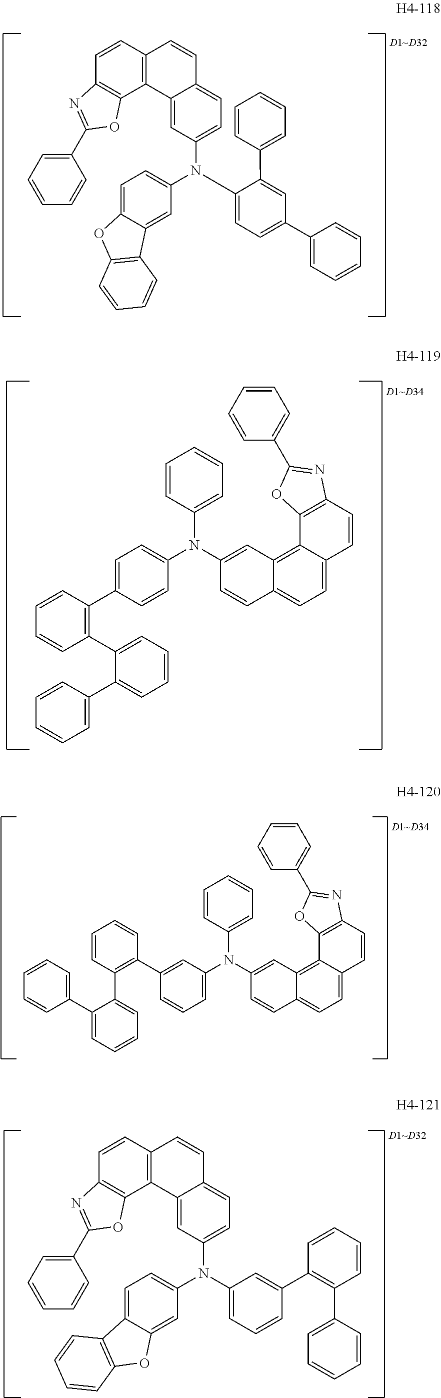

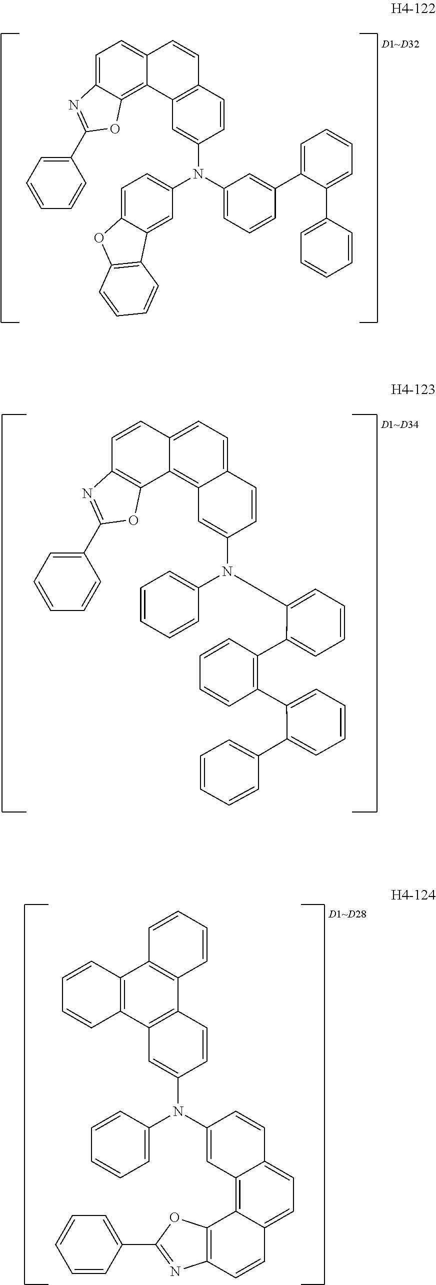

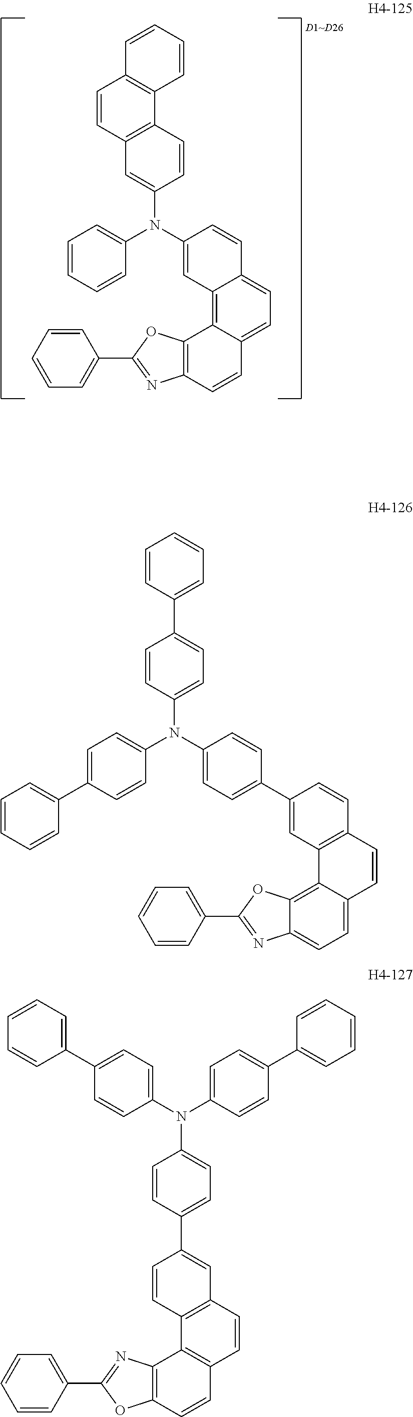

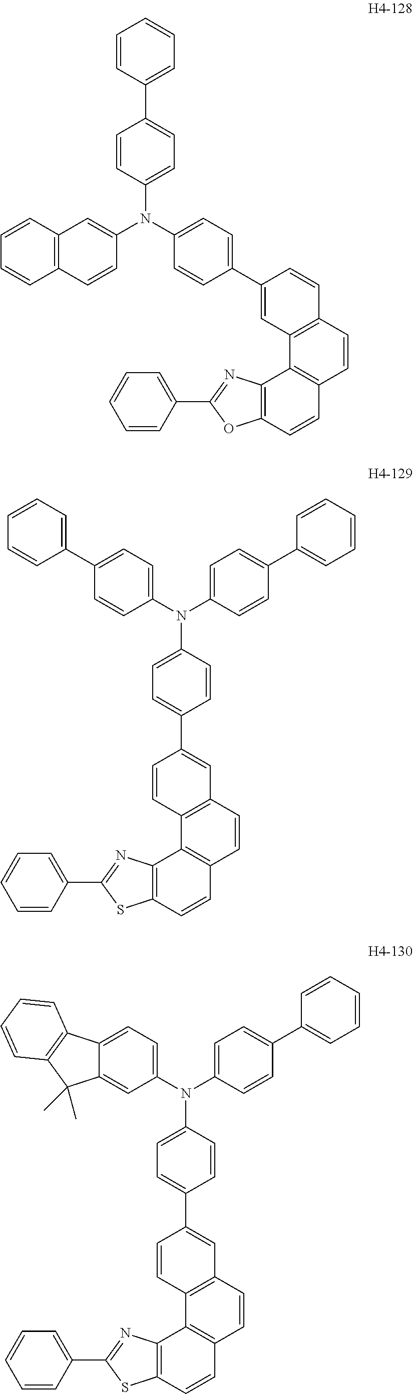 [SMILES: c1ccc(-c2ccc(N(c3ccc(-c4ccc5ccc6ccc7oc(-c8ccccc8)nc7c6c5c4)cc3)c3ccc4ccccc4c3)cc2)cc1 ; c1ccc(-c2ccc(N(c3ccc(-c4ccccc4)cc3)c3ccc(-c4ccc5c(ccc6ccc7sc(-c8ccccc8)nc7c65)c4)cc3)cc2)cc1 ; CC1(C)c2ccccc2-c2ccc(N(c3ccc(-c4ccccc4)cc3)c3ccc(-c4ccc5c(ccc6ccc7sc(-c8ccccc8)nc7c65)c4)cc3)cc21]

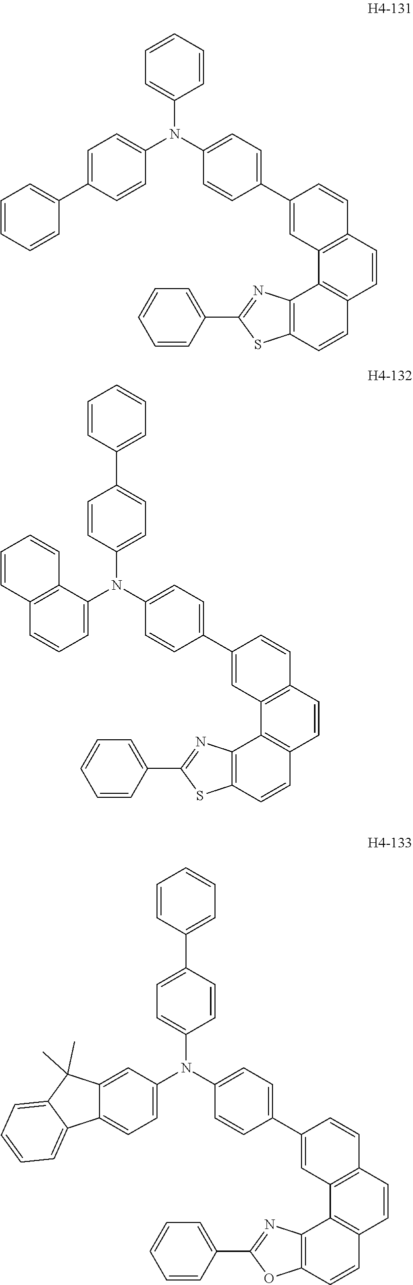 [SMILES: c1ccc(-c2ccc(N(c3ccccc3)c3ccc(-c4ccc5ccc6ccc7sc(-c8ccccc8)nc7c6c5c4)cc3)cc2)cc1 ; c1ccc(-c2ccc(N(c3ccc(-c4ccc5ccc6ccc7sc(-c8ccccc8)nc7c6c5c4)cc3)c3cccc4ccccc34)cc2)cc1 ; CC1(C)c2ccccc2-c2ccc(N(c3ccc(-c4ccccc4)cc3)c3ccc(-c4ccc5ccc6ccc7oc(-c8ccccc8)nc7c6c5c4)cc3)cc21]

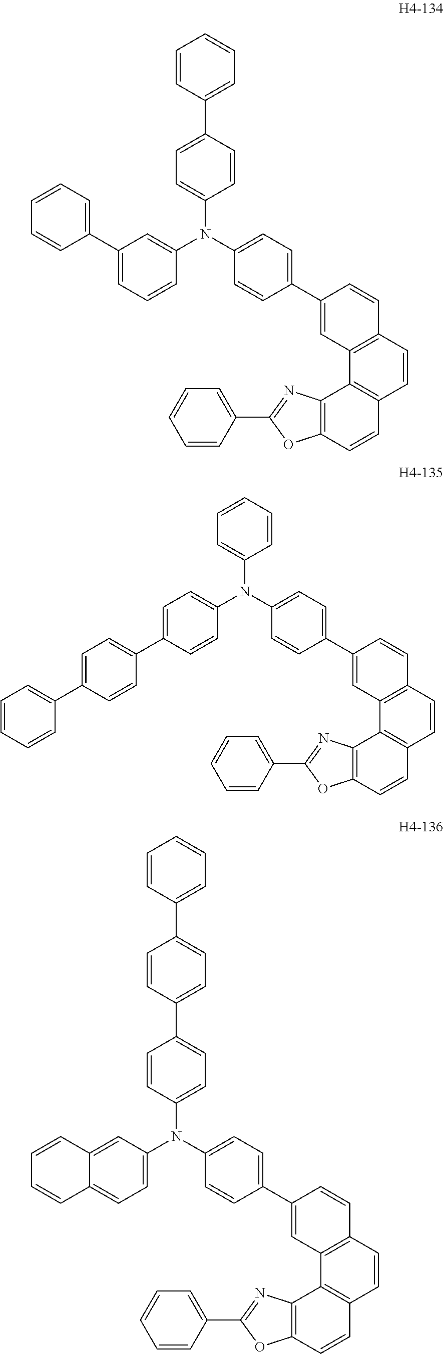 [SMILES: c1ccc(-c2ccc(N(c3ccc(-c4ccc5ccc6ccc7oc(-c8ccccc8)nc7c6c5c4)cc3)c3cccc(-c4ccccc4)c3)cc2)cc1 ; c1ccc(-c2ccc(-c3ccc(N(c4ccccc4)c4ccc(-c5ccc6ccc7ccc8oc(-c9ccccc9)nc8c7c6c5)cc4)cc3)cc2)cc1 ; c1ccc(-c2ccc(-c3ccc(N(c4ccc(-c5ccc6ccc7ccc8oc(-c9ccccc9)nc8c7c6c5)cc4)c4ccc5ccccc5c4)cc3)cc2)cc1]

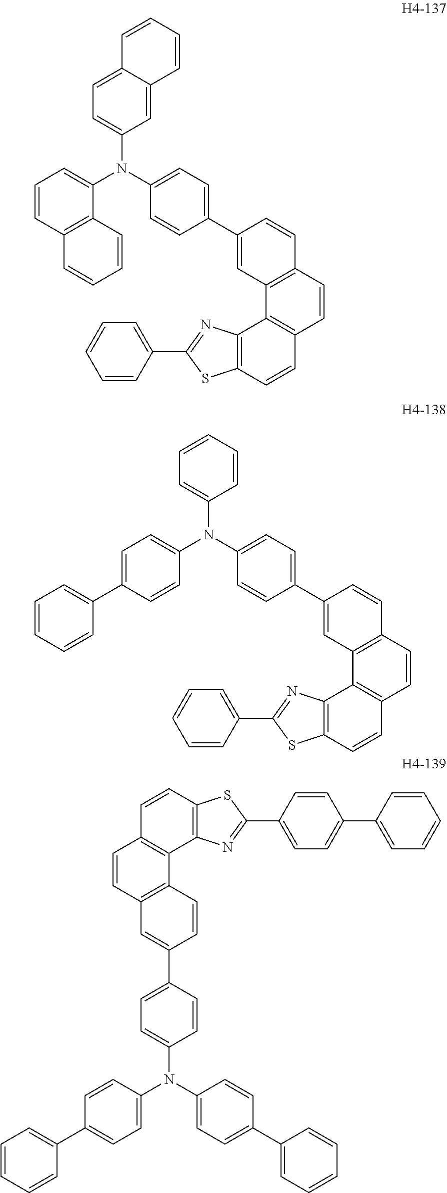

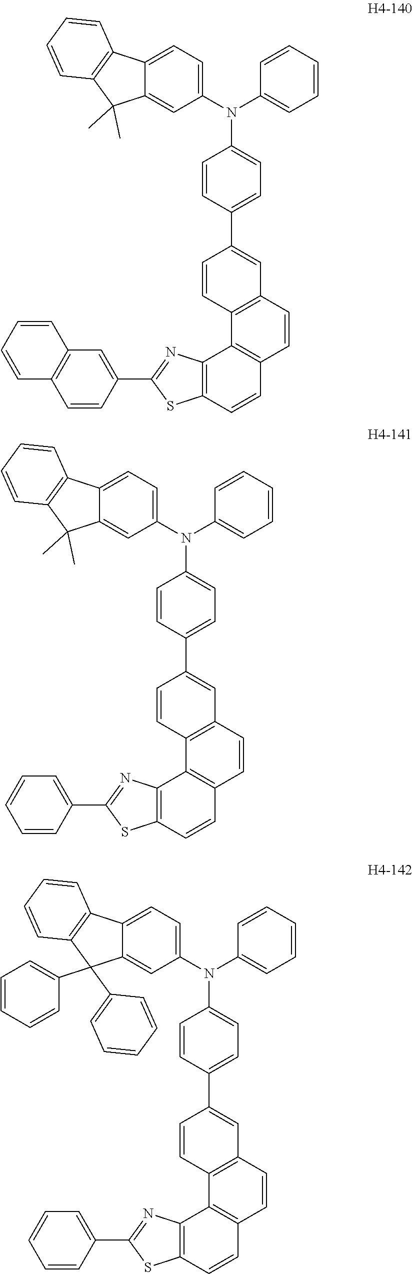

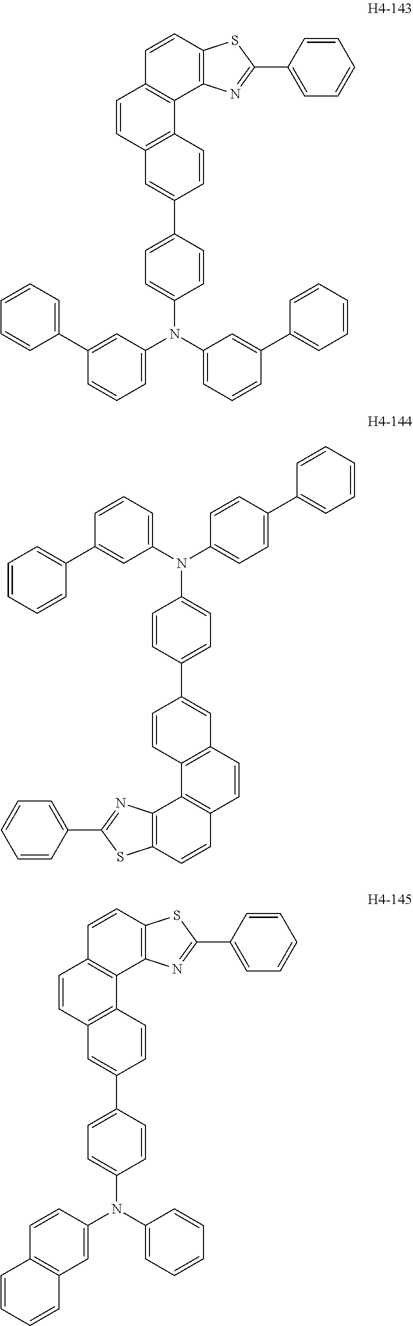 [SMILES: c1ccc(-c2cccc(N(c3ccc(-c4ccc5c(ccc6ccc7sc(-c8ccccc8)nc7c65)c4)cc3)c3cccc(-c4ccccc4)c3)c2)cc1 ; c1ccc(-c2ccc(N(c3ccc(-c4ccc5c(ccc6ccc7sc(-c8ccccc8)nc7c65)c4)cc3)c3cccc(-c4ccccc4)c3)cc2)cc1 ; c1ccc(-c2nc3c(ccc4ccc5cc(-c6ccc(N(c7ccccc7)c7ccc8ccccc8c7)cc6)ccc5c43)s2)cc1]

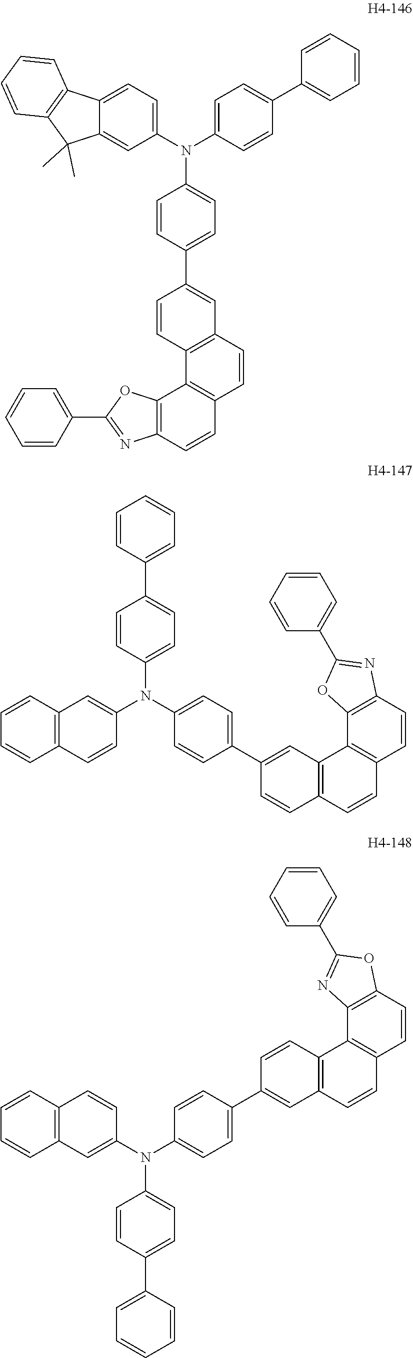 [SMILES: CC1(C)c2ccccc2-c2ccc(N(c3ccc(-c4ccccc4)cc3)c3ccc(-c4ccc5c(ccc6ccc7nc(-c8ccccc8)oc7c65)c4)cc3)cc21 ; c1ccc(-c2ccc(N(c3ccc(-c4ccc5ccc6ccc7nc(-c8ccccc8)oc7c6c5c4)cc3)c3ccc4ccccc4c3)cc2)cc1 ; c1ccc(-c2ccc(N(c3ccc(-c4ccc5c(ccc6ccc7oc(-c8ccccc8)nc7c65)c4)cc3)c3ccc4ccccc4c3)cc2)cc1]

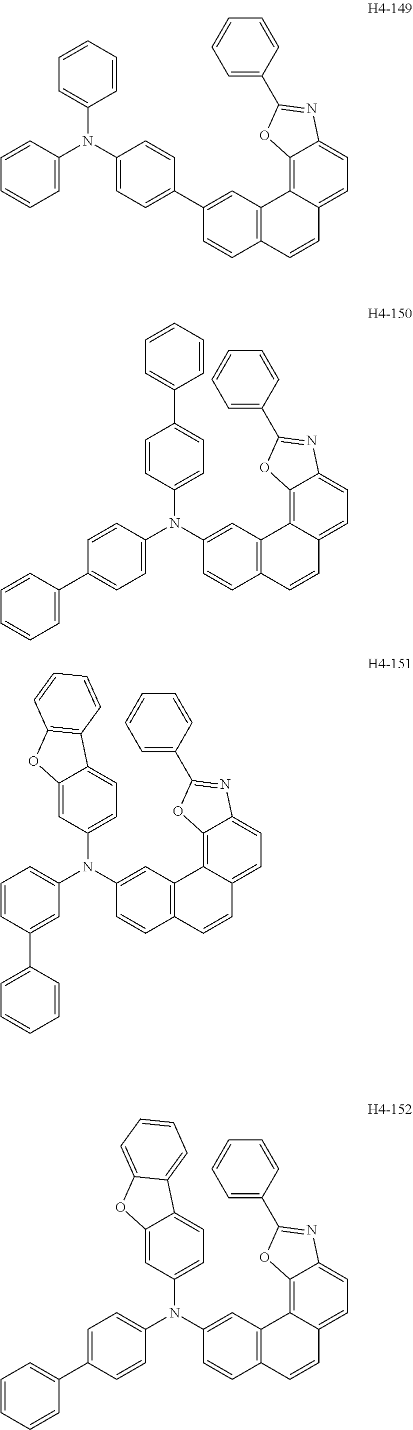

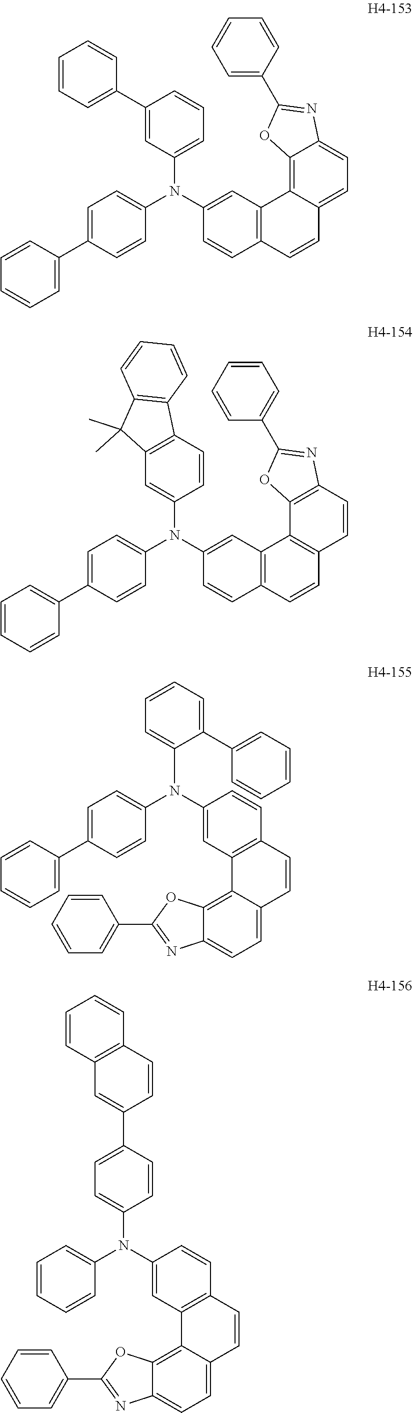

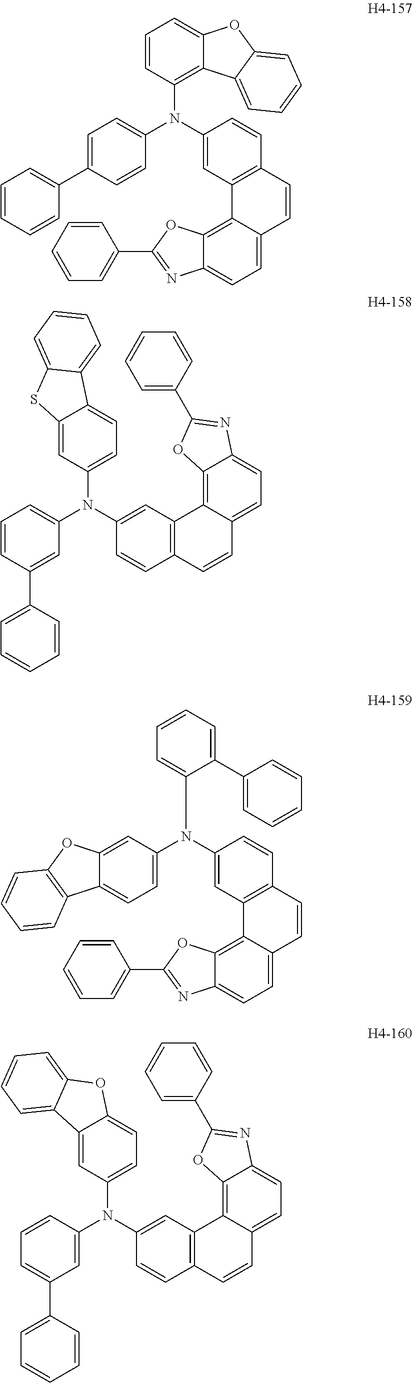

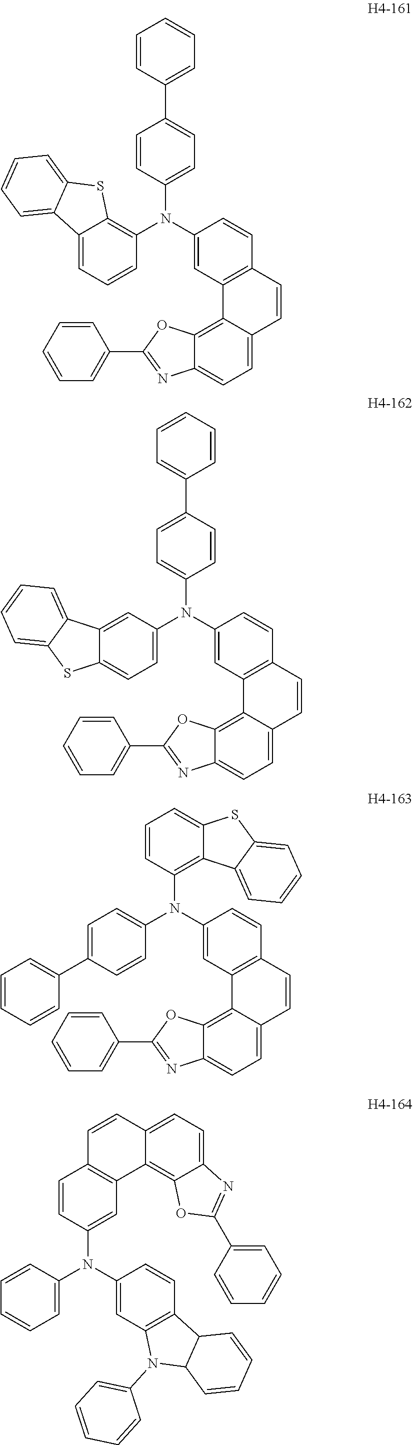

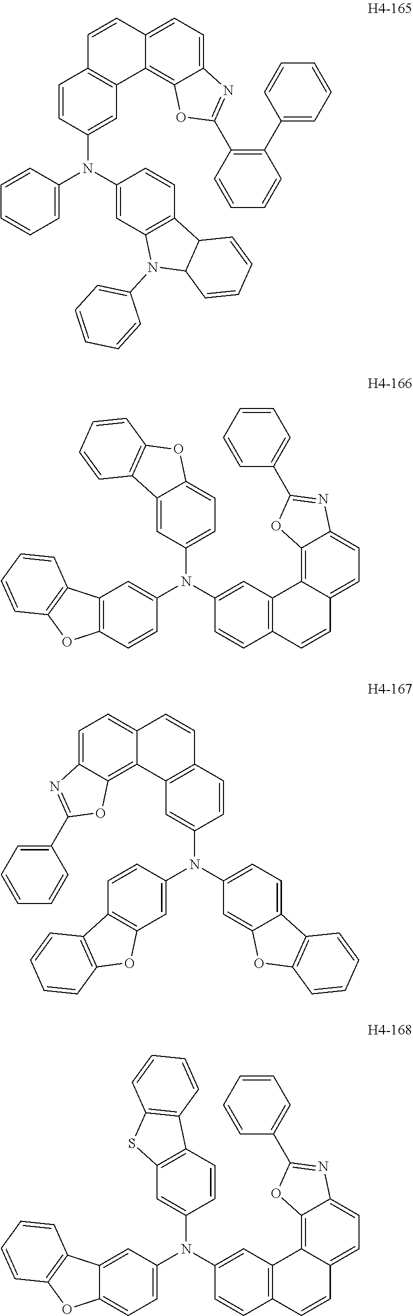 [SMILES: C1=CC2c3ccc(N(c4ccccc4)c4ccc5ccc6ccc7nc(-c8ccccc8-c8ccccc8)oc7c6c5c4)cc3N(c3ccccc3)C2C=C1 ; c1ccc(-c2nc3ccc4ccc5ccc(N(c6ccc7oc8ccccc8c7c6)c6ccc7oc8ccccc8c7c6)cc5c4c3o2)cc1 ; c1ccc(-c2nc3ccc4ccc5ccc(N(c6ccc7c(c6)oc6ccccc67)c6ccc7c(c6)oc6ccccc67)cc5c4c3o2)cc1 ; c1ccc(-c2nc3ccc4ccc5ccc(N(c6ccc7c(c6)sc6ccccc67)c6ccc7oc8ccccc8c7c6)cc5c4c3o2)cc1]

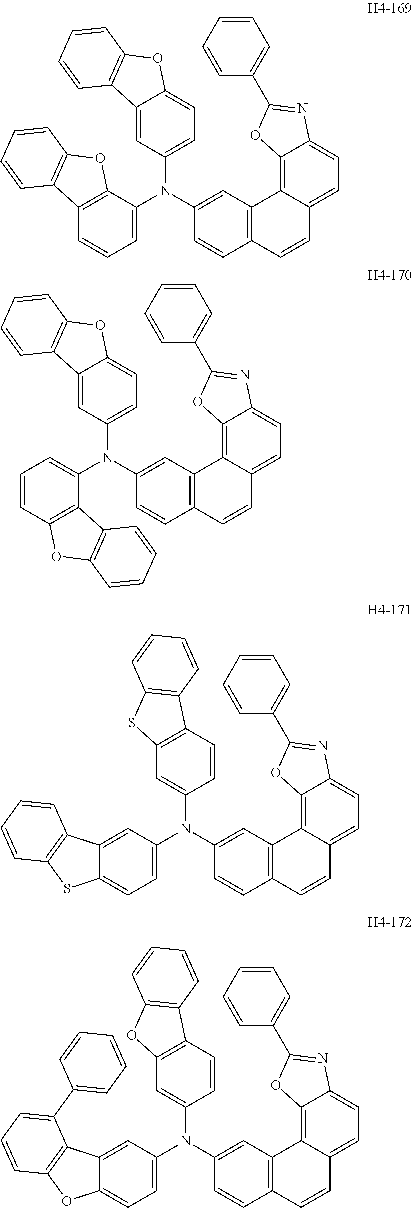

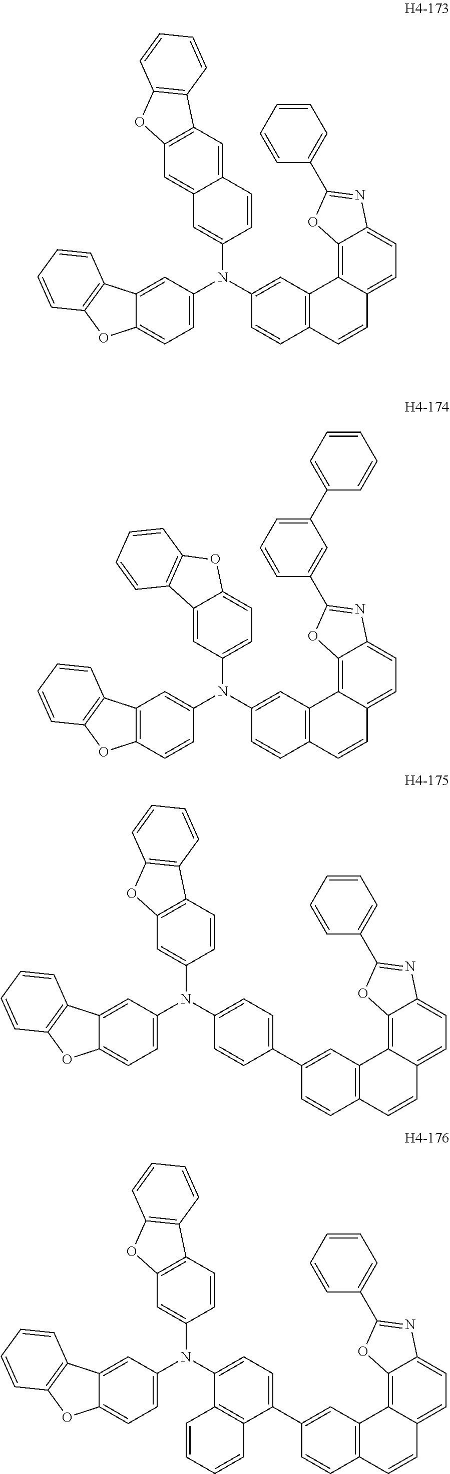 [SMILES: c1ccc(-c2nc3ccc4ccc5ccc(N(c6ccc7cc8c(cc7c6)oc6ccccc68)c6ccc7oc8ccccc8c7c6)cc5c4c3o2)cc1 ; c1ccc(-c2cccc(-c3nc4ccc5ccc6ccc(N(c7ccc8oc9ccccc9c8c7)c7ccc8oc9ccccc9c8c7)cc6c5c4o3)c2)cc1 ; c1ccc(-c2nc3ccc4ccc5ccc(-c6ccc(N(c7ccc8c(c7)oc7ccccc78)c7ccc8oc9ccccc9c8c7)cc6)cc5c4c3o2)cc1 ; c1ccc(-c2nc3ccc4ccc5ccc(-c6ccc(N(c7ccc8c(c7)oc7ccccc78)c7ccc8oc9ccccc9c8c7)c7ccccc67)cc5c4c3o2)cc1]

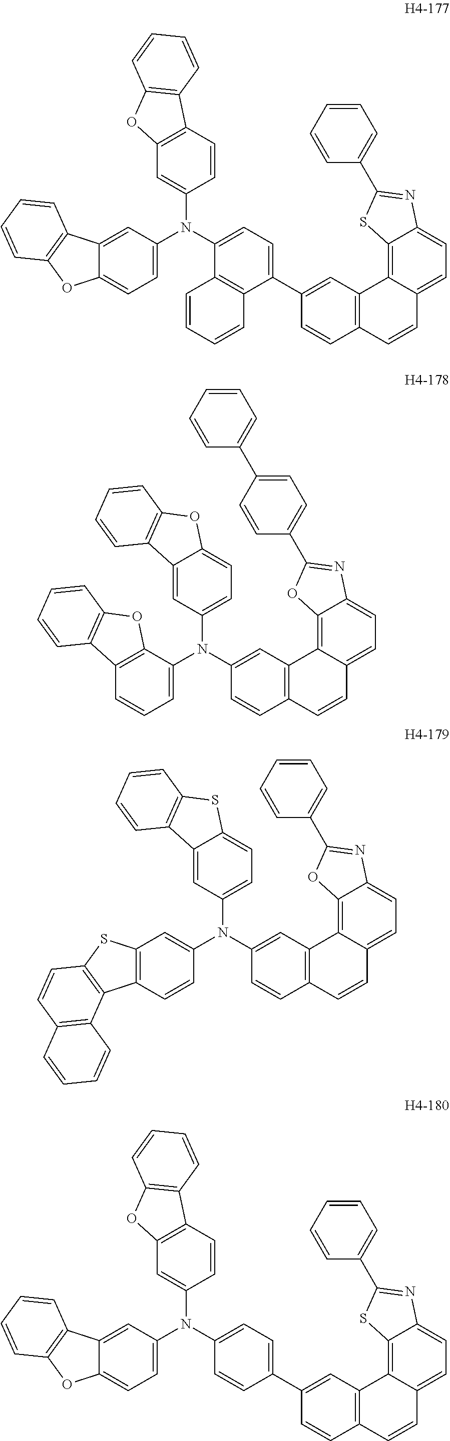

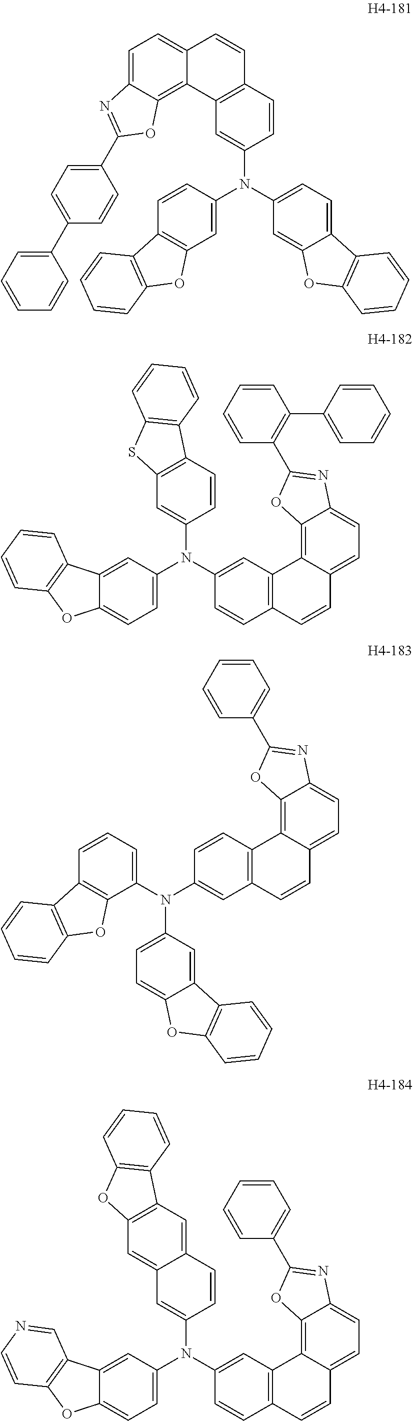

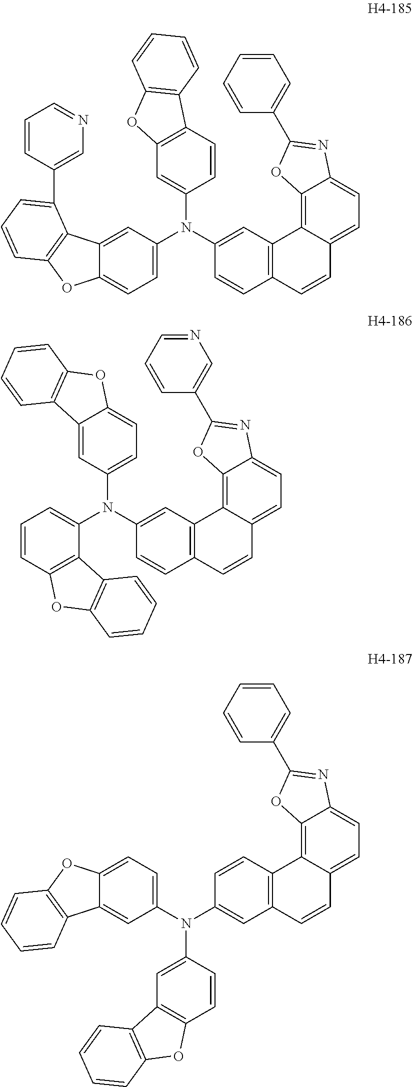 [SMILES: c1ccc(-c2nc3ccc4ccc5ccc(N(c6ccc7c(c6)oc6ccccc67)c6ccc7oc8cccc(-c9cccnc9)c8c7c6)cc5c4c3o2)cc1 ; c1cncc(-c2nc3ccc4ccc5ccc(N(c6ccc7oc8ccccc8c7c6)c6cccc7oc8ccccc8c67)cc5c4c3o2)c1 ; c1ccc(-c2nc3ccc4ccc5cc(N(c6ccc7oc8ccccc8c7c6)c6ccc7oc8ccccc8c7c6)ccc5c4c3o2)cc1]

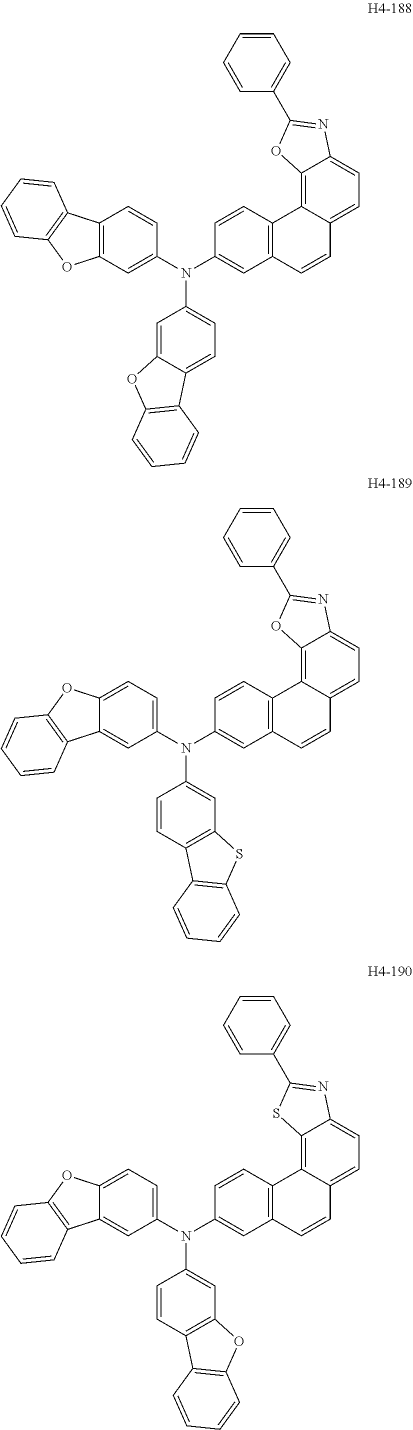 [SMILES: c1ccc(-c2nc3ccc4ccc5cc(N(c6ccc7c(c6)oc6ccccc67)c6ccc7c(c6)oc6ccccc67)ccc5c4c3o2)cc1 ; c1ccc(-c2nc3ccc4ccc5cc(N(c6ccc7c(c6)sc6ccccc67)c6ccc7oc8ccccc8c7c6)ccc5c4c3o2)cc1 ; c1ccc(-c2nc3ccc4ccc5cc(N(c6ccc7c(c6)oc6ccccc67)c6ccc7oc8ccccc8c7c6)ccc5c4c3s2)cc1]

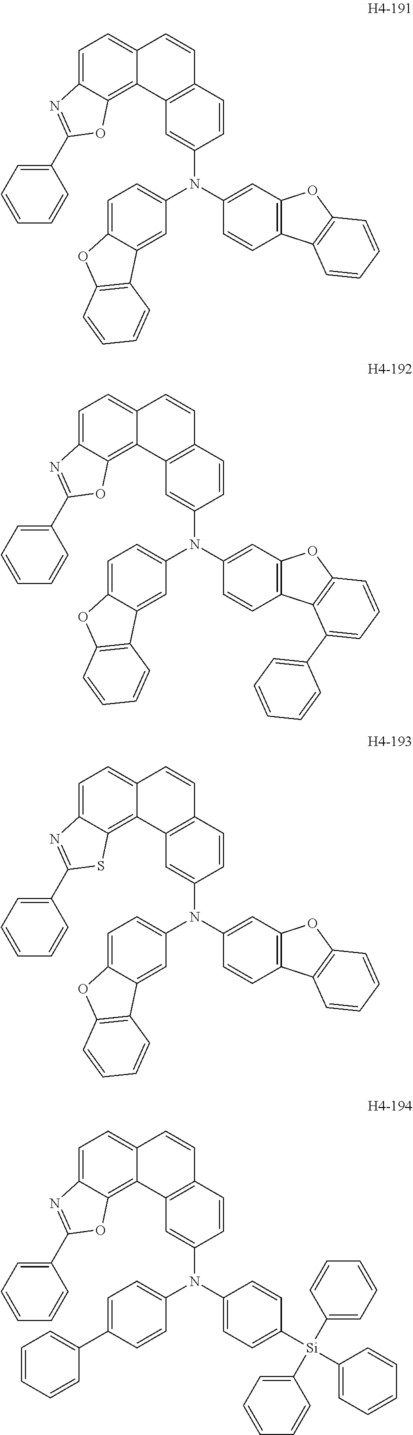 [SMILES: c1ccc(-c2nc3ccc4ccc5ccc(N(c6ccc7c(c6)oc6ccccc67)c6ccc7oc8ccccc8c7c6)cc5c4c3o2)cc1 ; c1ccc(-c2nc3ccc4ccc5ccc(N(c6ccc7c(c6)oc6cccc(-c8ccccc8)c67)c6ccc7oc8ccccc8c7c6)cc5c4c3o2)cc1 ; c1ccc(-c2nc3ccc4ccc5ccc(N(c6ccc7c(c6)oc6ccccc67)c6ccc7oc8ccccc8c7c6)cc5c4c3s2)cc1 ; c1ccc(-c2ccc(N(c3ccc([Si](c4ccccc4)(c4ccccc4)c4ccccc4)cc3)c3ccc4ccc5ccc6nc(-c7ccccc7)oc6c5c4c3)cc2)cc1]

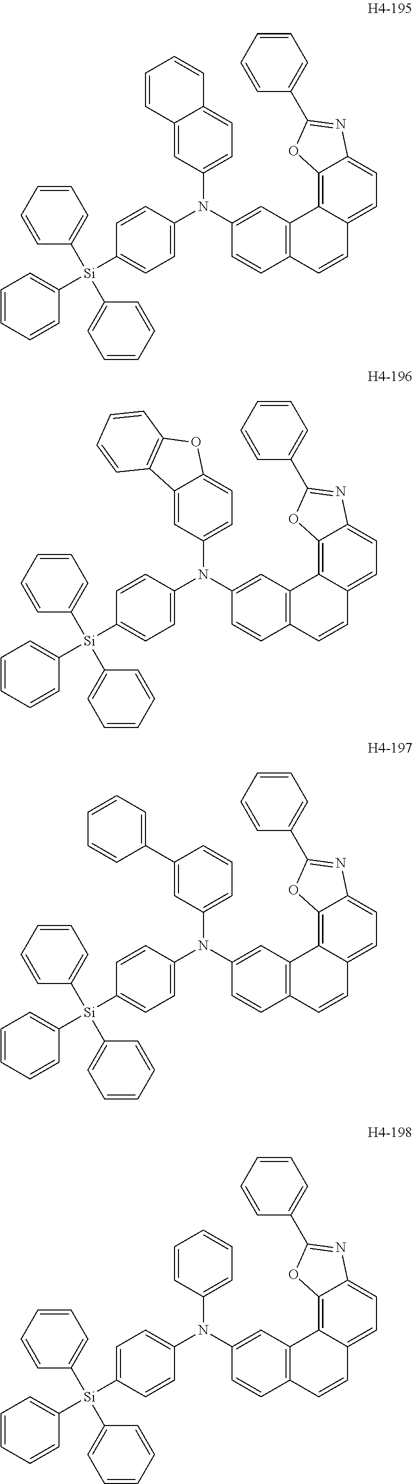 [SMILES: c1ccc(-c2nc3ccc4ccc5ccc(N(c6ccc([Si](c7ccccc7)(c7ccccc7)c7ccccc7)cc6)c6ccc7ccccc7c6)cc5c4c3o2)cc1 ; c1ccc(-c2nc3ccc4ccc5ccc(N(c6ccc([Si](c7ccccc7)(c7ccccc7)c7ccccc7)cc6)c6ccc7oc8ccccc8c7c6)cc5c4c3o2)cc1 ; c1ccc(-c2cccc(N(c3ccc([Si](c4ccccc4)(c4ccccc4)c4ccccc4)cc3)c3ccc4ccc5ccc6nc(-c7ccccc7)oc6c5c4c3)c2)cc1 ; c1ccc(-c2nc3ccc4ccc5ccc(N(c6ccccc6)c6ccc([Si](c7ccccc7)(c7ccccc7)c7ccccc7)cc6)cc5c4c3o2)cc1]

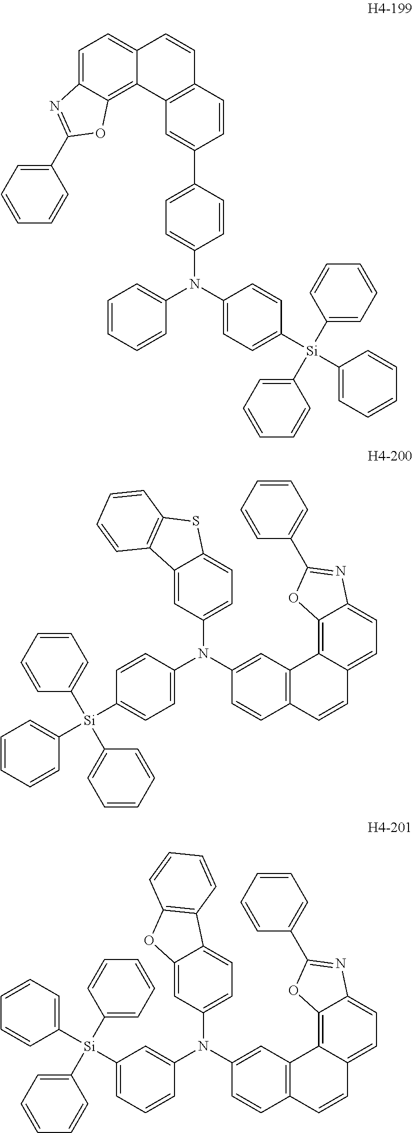

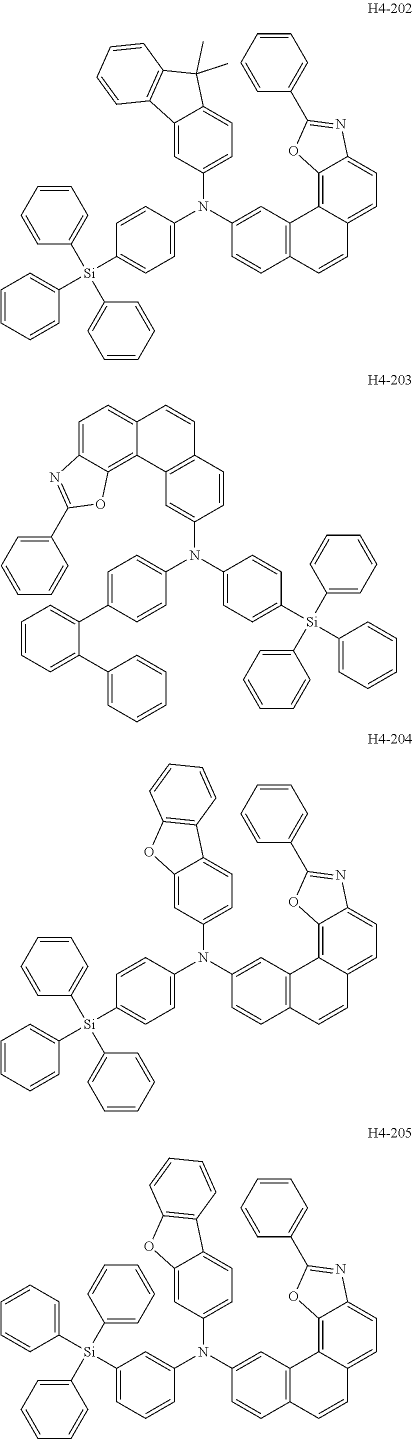

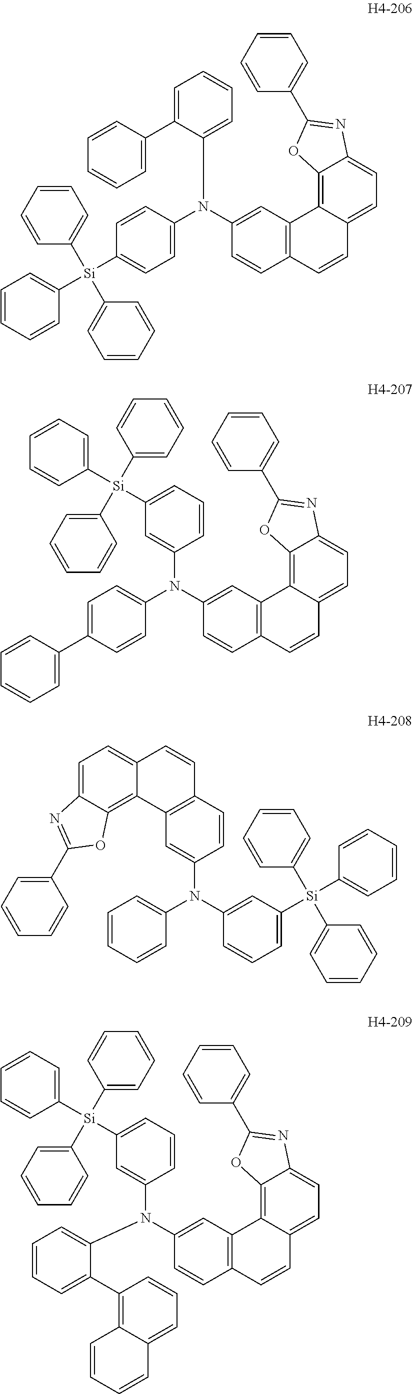

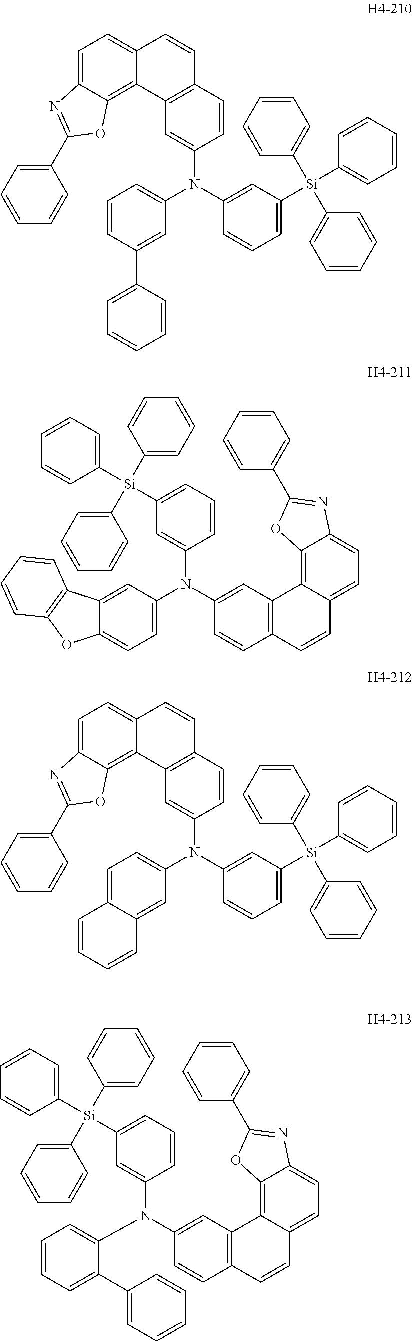 [SMILES: c1ccc(-c2cccc(N(c3cccc([Si](c4ccccc4)(c4ccccc4)c4ccccc4)c3)c3ccc4ccc5ccc6nc(-c7ccccc7)oc6c5c4c3)c2)cc1 ; c1ccc(-c2nc3ccc4ccc5ccc(N(c6cccc([Si](c7ccccc7)(c7ccccc7)c7ccccc7)c6)c6ccc7oc8ccccc8c7c6)cc5c4c3o2)cc1 ; c1ccc(-c2nc3ccc4ccc5ccc(N(c6cccc([Si](c7ccccc7)(c7ccccc7)c7ccccc7)c6)c6ccc7ccccc7c6)cc5c4c3o2)cc1 ; c1ccc(-c2nc3ccc4ccc5ccc(N(c6cccc([Si](c7ccccc7)(c7ccccc7)c7ccccc7)c6)c6ccccc6-c6ccccc6)cc5c4c3o2)cc1]

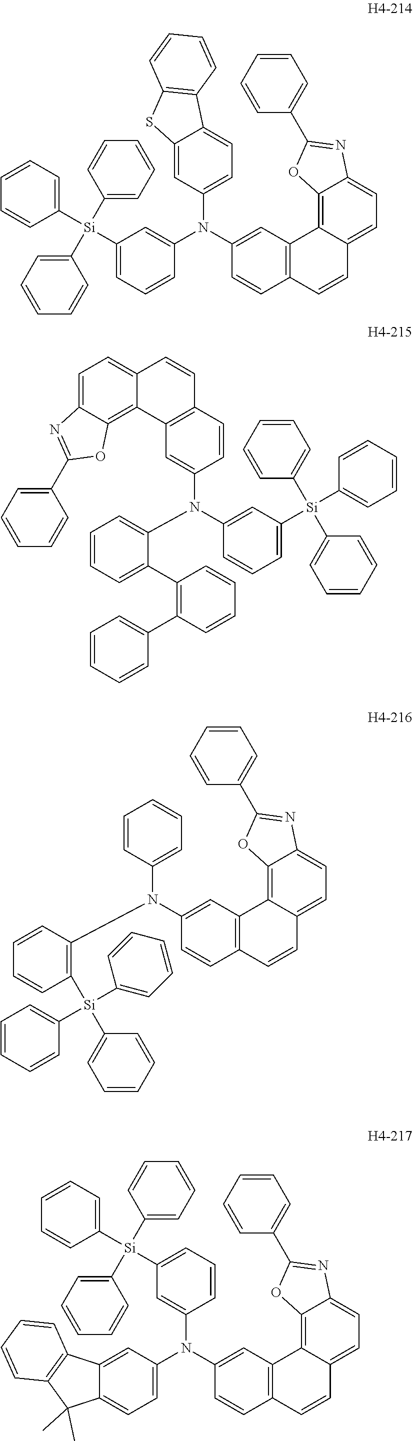 [SMILES: c1ccc(-c2nc3ccc4ccc5ccc(N(c6cccc([Si](c7ccccc7)(c7ccccc7)c7ccccc7)c6)c6ccc7c(c6)sc6ccccc67)cc5c4c3o2)cc1 ; c1ccc(-c2nc3ccc4ccc5ccc(N(c6cccc([Si](c7ccccc7)(c7ccccc7)c7ccccc7)c6)c6ccccc6-c6ccccc6-c6ccccc6)cc5c4c3o2)cc1 ; c1ccc(-c2nc3ccc4ccc5ccc(N(c6ccccc6)c6ccccc6[Si](c6ccccc6)(c6ccccc6)c6ccccc6)cc5c4c3o2)cc1 ; CC1(C)c2ccccc2-c2cc(N(c3cccc([Si](c4ccccc4)(c4ccccc4)c4ccccc4)c3)c3ccc4ccc5ccc6nc(-c7ccccc7)oc6c5c4c3)ccc21]

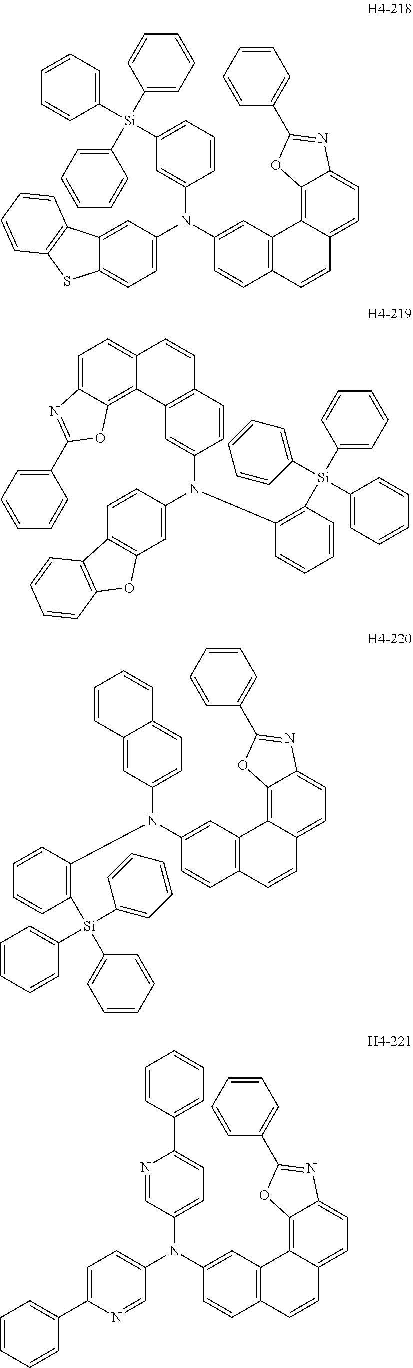 [SMILES: c1ccc(-c2nc3ccc4ccc5ccc(N(c6cccc([Si](c7ccccc7)(c7ccccc7)c7ccccc7)c6)c6ccc7sc8ccccc8c7c6)cc5c4c3o2)cc1 ; c1ccc(-c2nc3ccc4ccc5ccc(N(c6ccc7c(c6)oc6ccccc67)c6ccccc6[Si](c6ccccc6)(c6ccccc6)c6ccccc6)cc5c4c3o2)cc1 ; c1ccc(-c2nc3ccc4ccc5ccc(N(c6ccc7ccccc7c6)c6ccccc6[Si](c6ccccc6)(c6ccccc6)c6ccccc6)cc5c4c3o2)cc1 ; c1ccc(-c2ccc(N(c3ccc(-c4ccccc4)nc3)c3ccc4ccc5ccc6nc(-c7ccccc7)oc6c5c4c3)cn2)cc1]

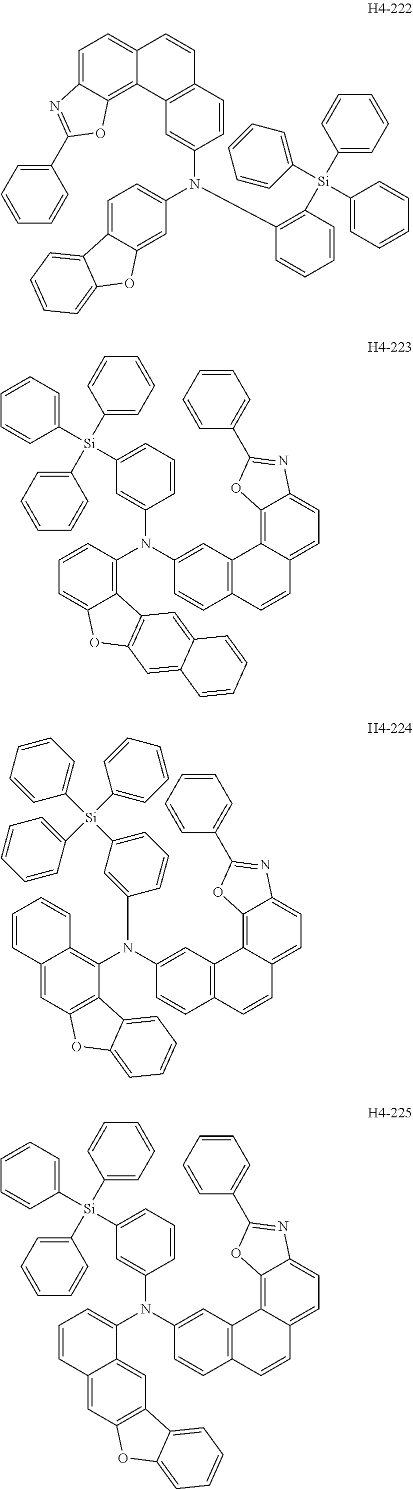

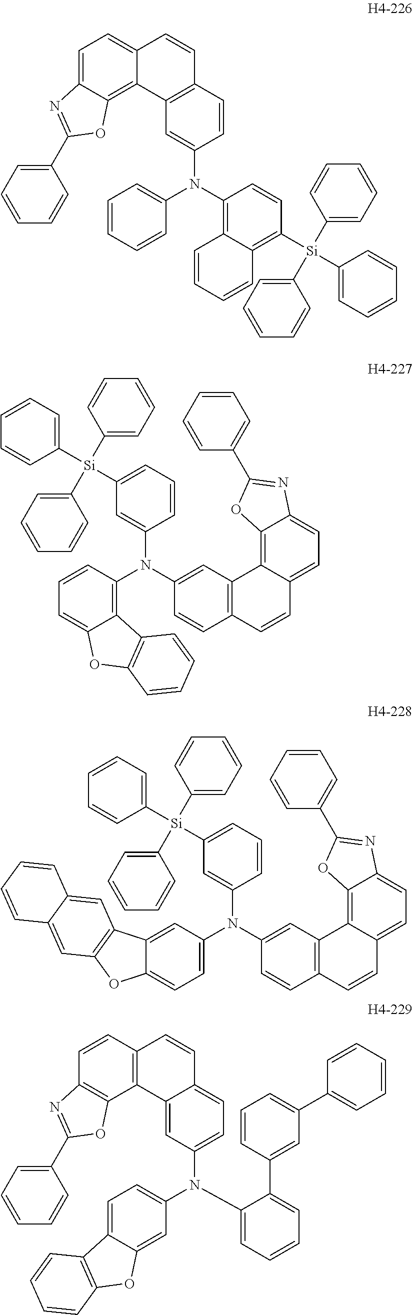

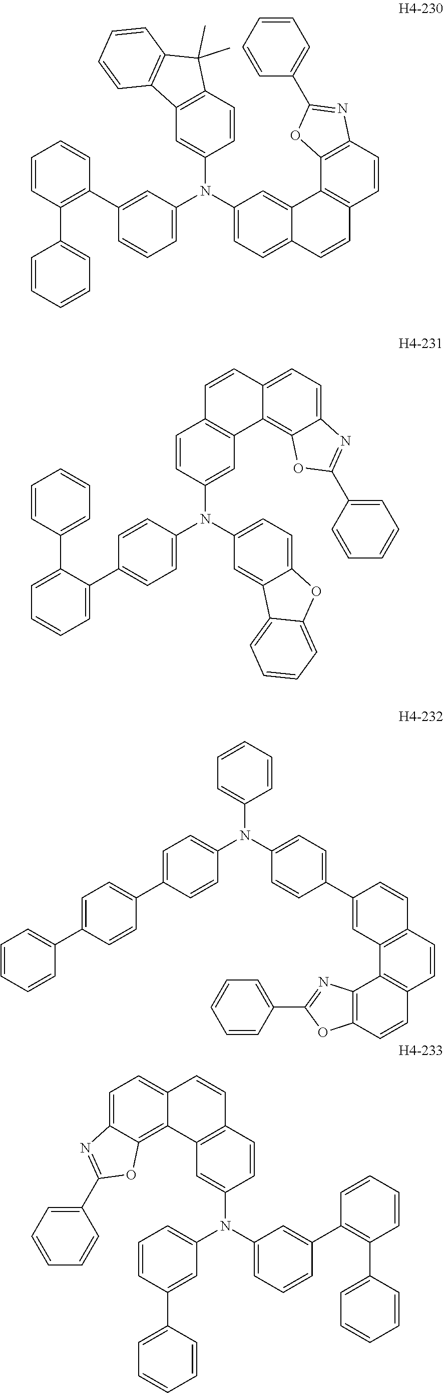 [SMILES: CC1(C)c2ccccc2-c2cc(N(c3cccc(-c4ccccc4-c4ccccc4)c3)c3ccc4ccc5ccc6nc(-c7ccccc7)oc6c5c4c3)ccc21 ; c1ccc(-c2nc3ccc4ccc5ccc(N(c6ccc(-c7ccccc7-c7ccccc7)cc6)c6ccc7oc8ccccc8c7c6)cc5c4c3o2)cc1 ; c1ccc(-c2ccc(-c3ccc(N(c4ccccc4)c4ccc(-c5ccc6ccc7ccc8oc(-c9ccccc9)nc8c7c6c5)cc4)cc3)cc2)cc1 ; c1ccc(-c2cccc(N(c3cccc(-c4ccccc4-c4ccccc4)c3)c3ccc4ccc5ccc6nc(-c7ccccc7)oc6c5c4c3)c2)cc1]

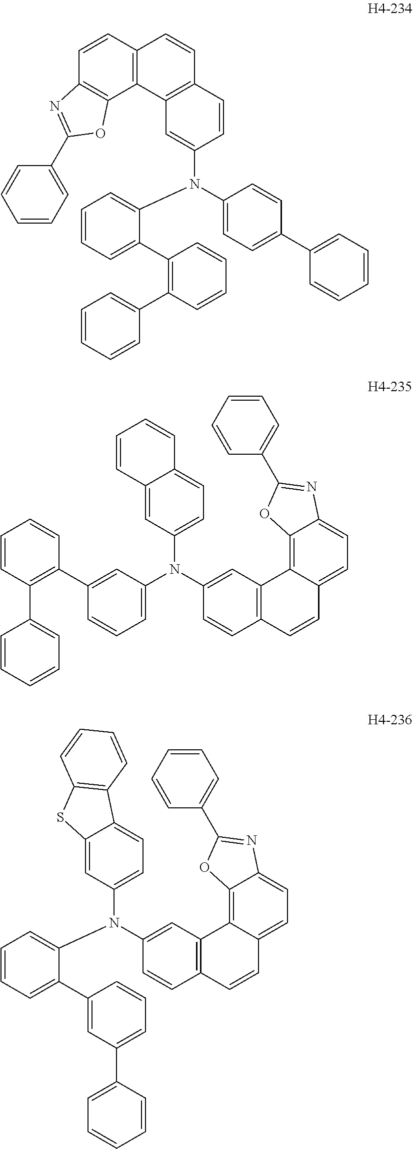 [SMILES: c1ccc(-c2ccc(N(c3ccc4ccc5ccc6nc(-c7ccccc7)oc6c5c4c3)c3ccccc3-c3ccccc3-c3ccccc3)cc2)cc1 ; c1ccc(-c2nc3ccc4ccc5ccc(N(c6cccc(-c7ccccc7-c7ccccc7)c6)c6ccc7ccccc7c6)cc5c4c3o2)cc1 ; c1ccc(-c2cccc(-c3ccccc3N(c3ccc4c(c3)sc3ccccc34)c3ccc4ccc5ccc6nc(-c7ccccc7)oc6c5c4c3)c2)cc1]

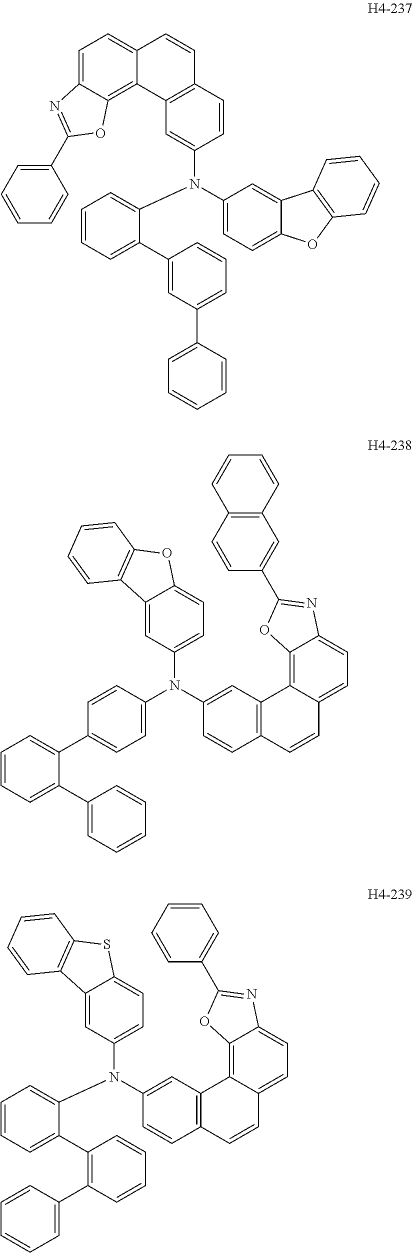 [SMILES: c1ccc(-c2cccc(-c3ccccc3N(c3ccc4oc5ccccc5c4c3)c3ccc4ccc5ccc6nc(-c7ccccc7)oc6c5c4c3)c2)cc1 ; c1ccc(-c2ccccc2-c2ccc(N(c3ccc4oc5ccccc5c4c3)c3ccc4ccc5ccc6nc(-c7ccc8ccccc8c7)oc6c5c4c3)cc2)cc1 ; c1ccc(-c2nc3ccc4ccc5ccc(N(c6ccc7sc8ccccc8c7c6)c6ccccc6-c6ccccc6-c6ccccc6)cc5c4c3o2)cc1]

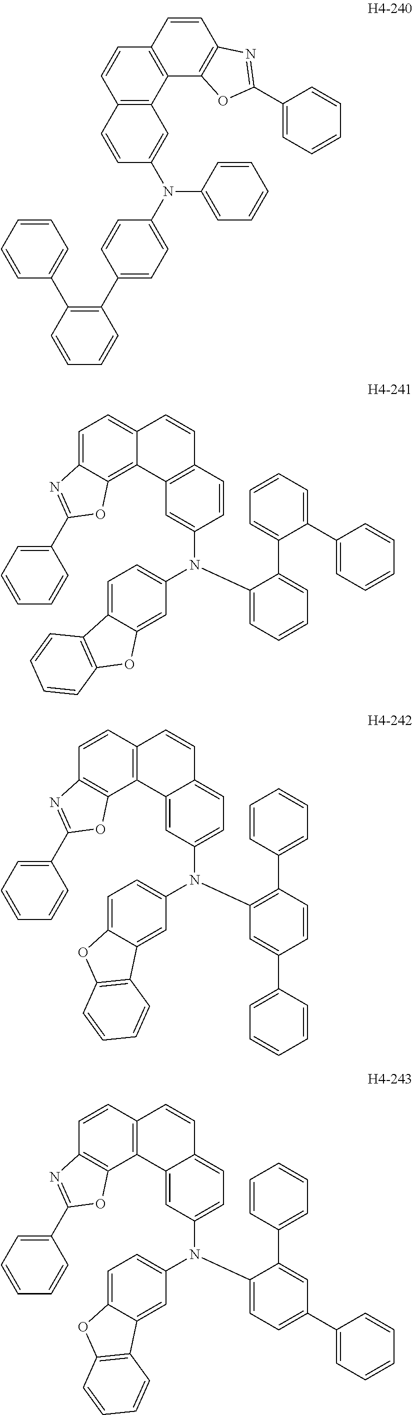 [SMILES: c1ccc(-c2nc3ccc4ccc5ccc(N(c6ccccc6)c6ccc(-c7ccccc7-c7ccccc7)cc6)cc5c4c3o2)cc1 ; c1ccc(-c2nc3ccc4ccc5ccc(N(c6ccc7c(c6)oc6ccccc67)c6ccccc6-c6ccccc6-c6ccccc6)cc5c4c3o2)cc1 ; c1ccc(-c2ccc(-c3ccccc3)c(N(c3ccc4oc5ccccc5c4c3)c3ccc4ccc5ccc6nc(-c7ccccc7)oc6c5c4c3)c2)cc1 ; c1ccc(-c2ccc(N(c3ccc4oc5ccccc5c4c3)c3ccc4ccc5ccc6nc(-c7ccccc7)oc6c5c4c3)c(-c3ccccc3)c2)cc1]

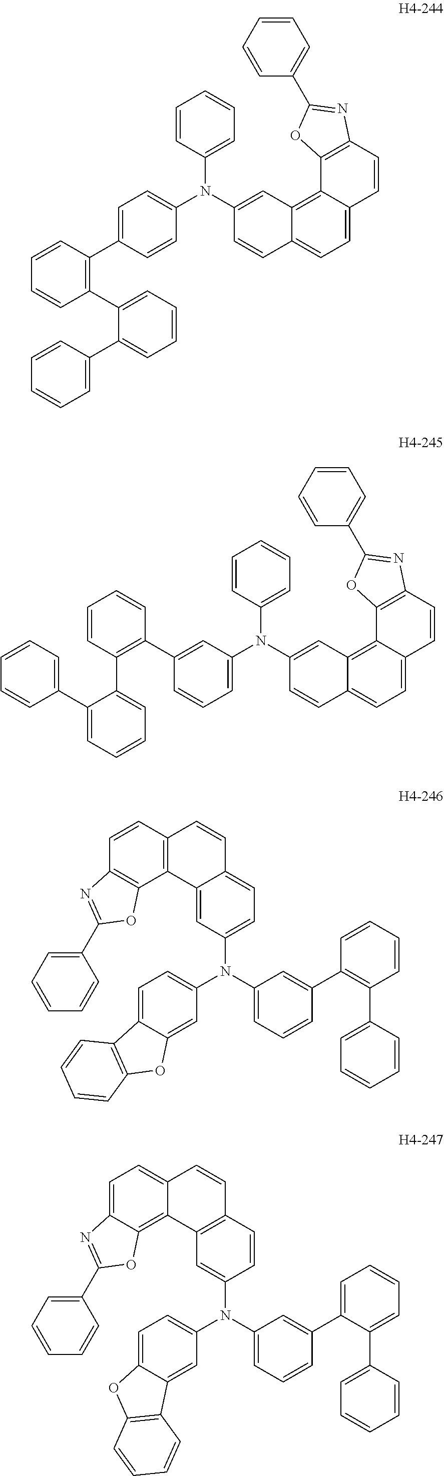 [SMILES: c1ccc(-c2nc3ccc4ccc5ccc(N(c6ccccc6)c6ccc(-c7ccccc7-c7ccccc7-c7ccccc7)cc6)cc5c4c3o2)cc1 ; c1ccc(-c2nc3ccc4ccc5ccc(N(c6ccccc6)c6cccc(-c7ccccc7-c7ccccc7-c7ccccc7)c6)cc5c4c3o2)cc1 ; c1ccc(-c2nc3ccc4ccc5ccc(N(c6cccc(-c7ccccc7-c7ccccc7)c6)c6ccc7c(c6)oc6ccccc67)cc5c4c3o2)cc1 ; c1ccc(-c2nc3ccc4ccc5ccc(N(c6cccc(-c7ccccc7-c7ccccc7)c6)c6ccc7oc8ccccc8c7c6)cc5c4c3o2)cc1]

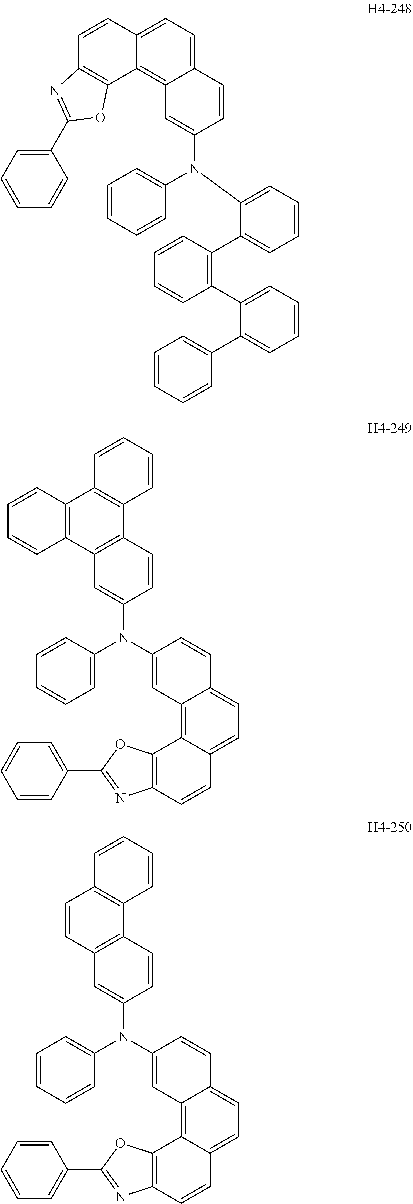

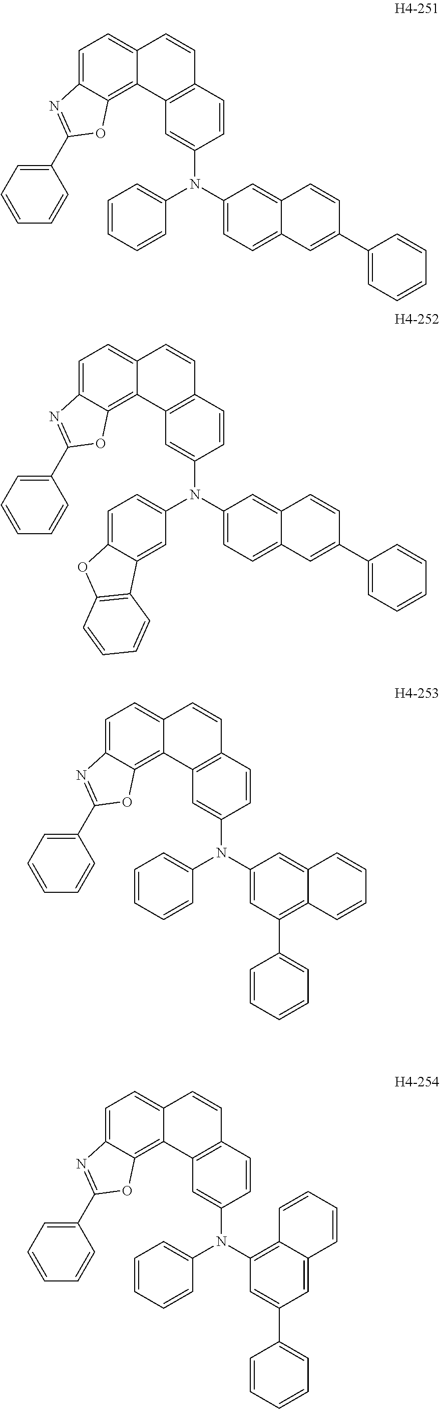 [SMILES: c1ccc(-c2ccc3cc(N(c4ccccc4)c4ccc5ccc6ccc7nc(-c8ccccc8)oc7c6c5c4)ccc3c2)cc1 ; c1ccc(-c2ccc3cc(N(c4ccc5oc6ccccc6c5c4)c4ccc5ccc6ccc7nc(-c8ccccc8)oc7c6c5c4)ccc3c2)cc1 ; c1ccc(-c2nc3ccc4ccc5ccc(N(c6ccccc6)c6cc(-c7ccccc7)c7ccccc7c6)cc5c4c3o2)cc1 ; c1ccc(-c2cc(N(c3ccccc3)c3ccc4ccc5ccc6nc(-c7ccccc7)oc6c5c4c3)c3ccccc3c2)cc1]

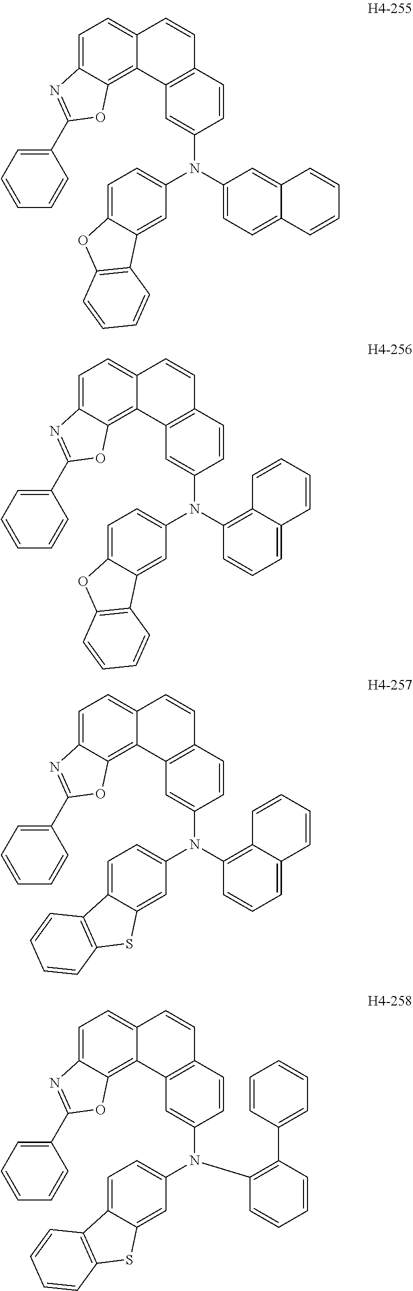

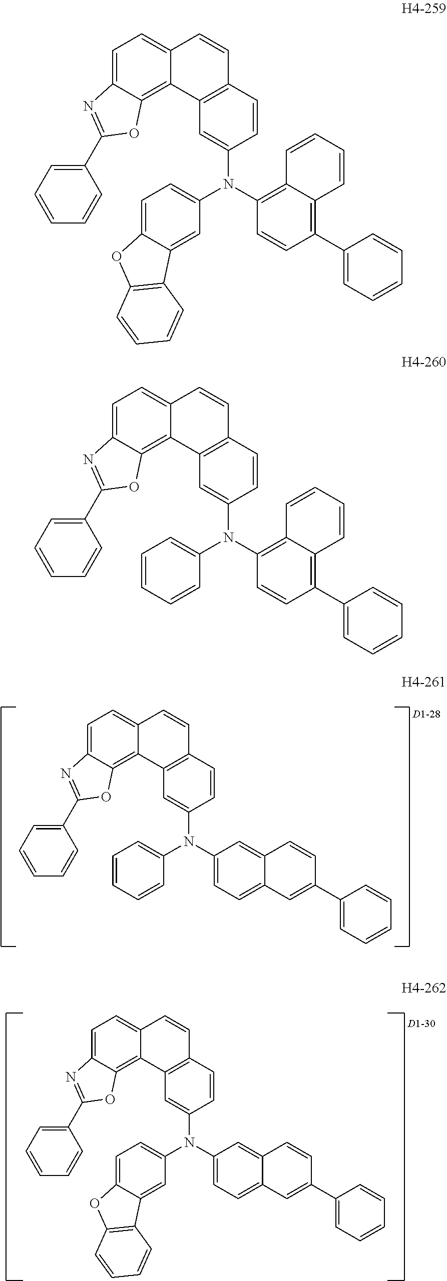

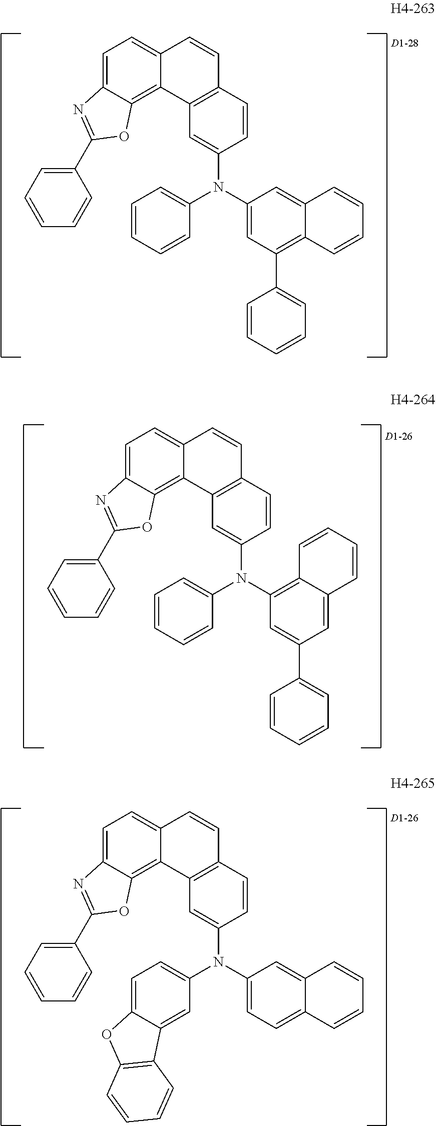

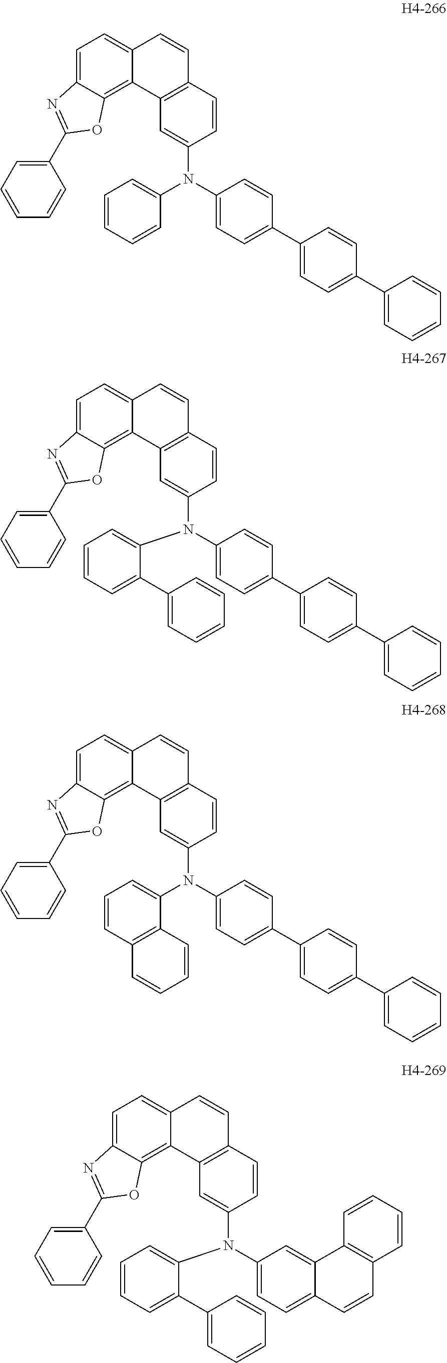 [SMILES: c1ccc(-c2ccc(-c3ccc(N(c4ccccc4)c4ccc5ccc6ccc7nc(-c8ccccc8)oc7c6c5c4)cc3)cc2)cc1 ; c1ccc(-c2ccc(-c3ccc(N(c4ccc5ccc6ccc7nc(-c8ccccc8)oc7c6c5c4)c4ccccc4-c4ccccc4)cc3)cc2)cc1 ; c1ccc(-c2ccc(-c3ccc(N(c4ccc5ccc6ccc7nc(-c8ccccc8)oc7c6c5c4)c4cccc5ccccc45)cc3)cc2)cc1 ; c1ccc(-c2nc3ccc4ccc5ccc(N(c6ccc7ccc8ccccc8c7c6)c6ccccc6-c6ccccc6)cc5c4c3o2)cc1]

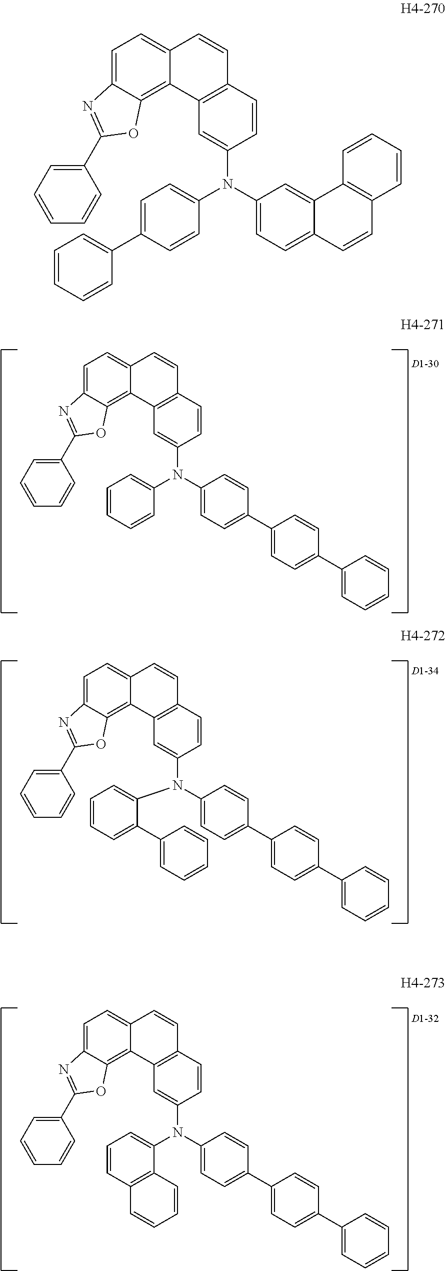

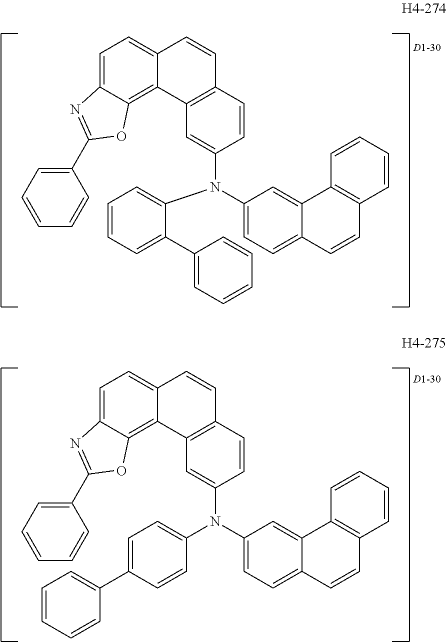

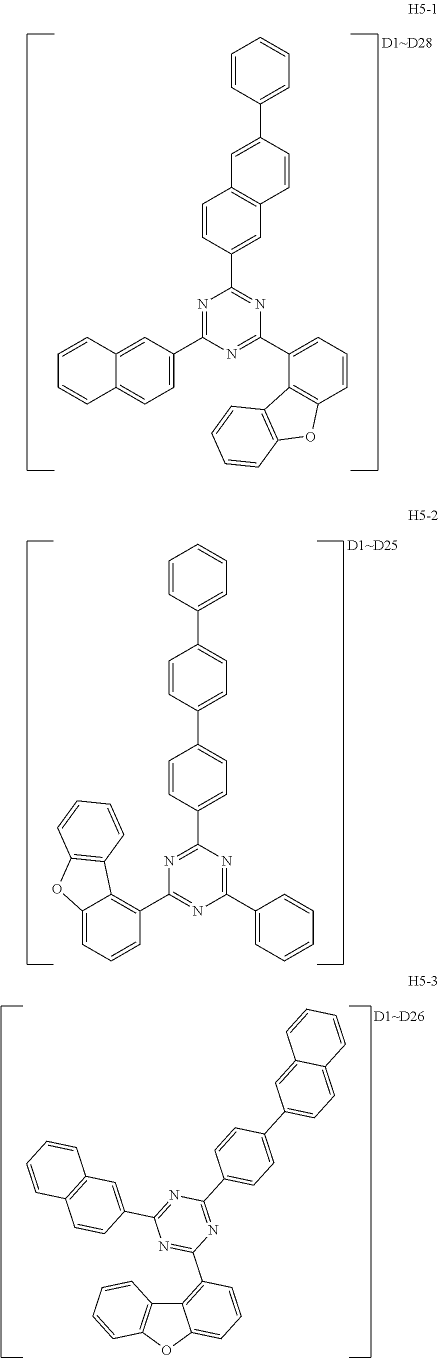

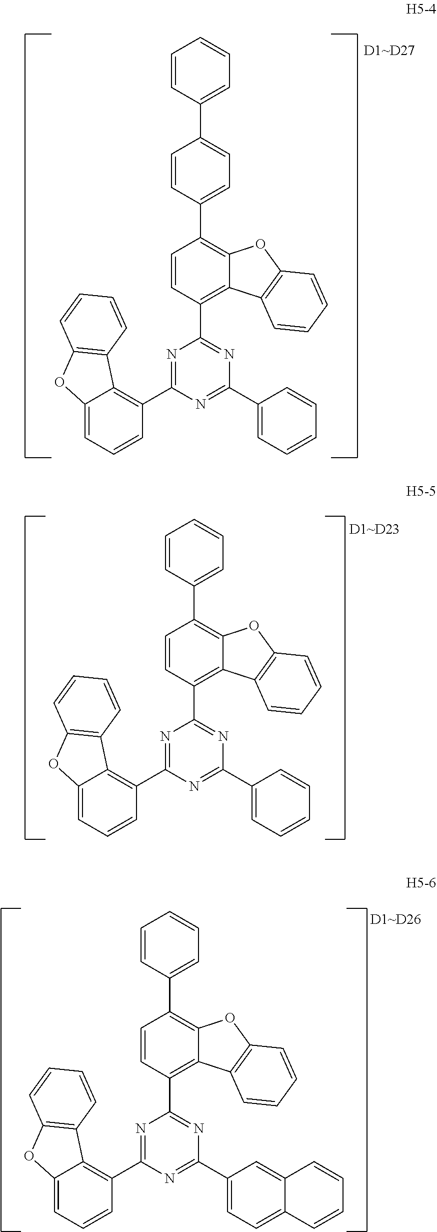

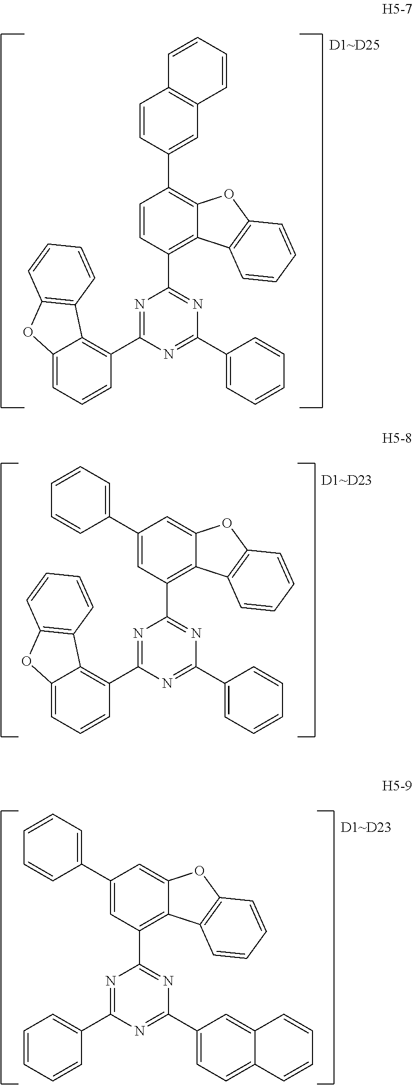

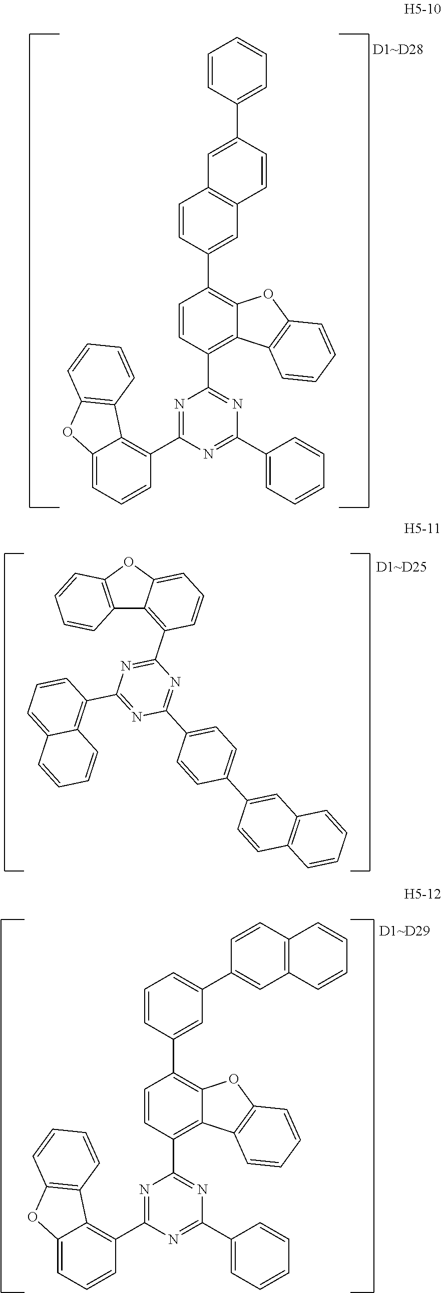

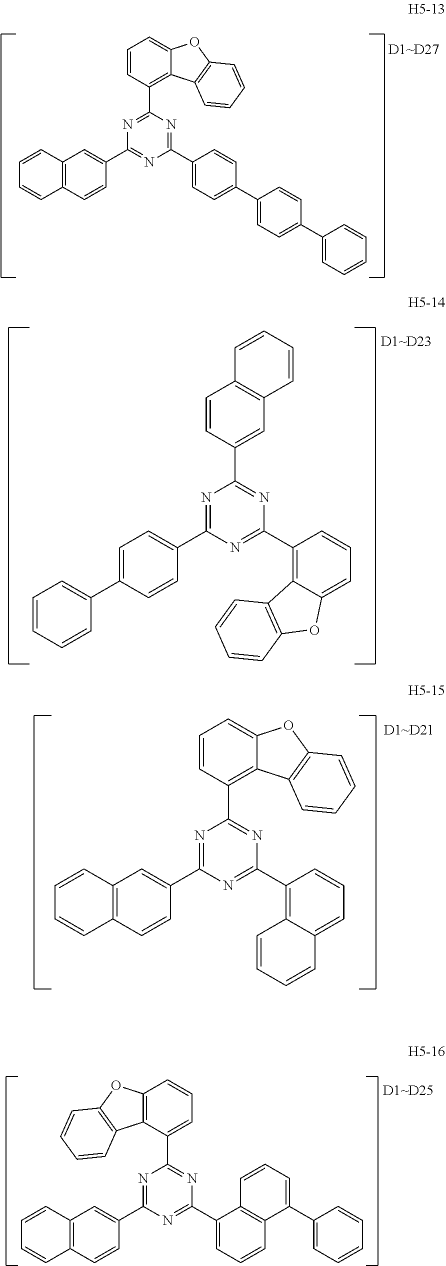

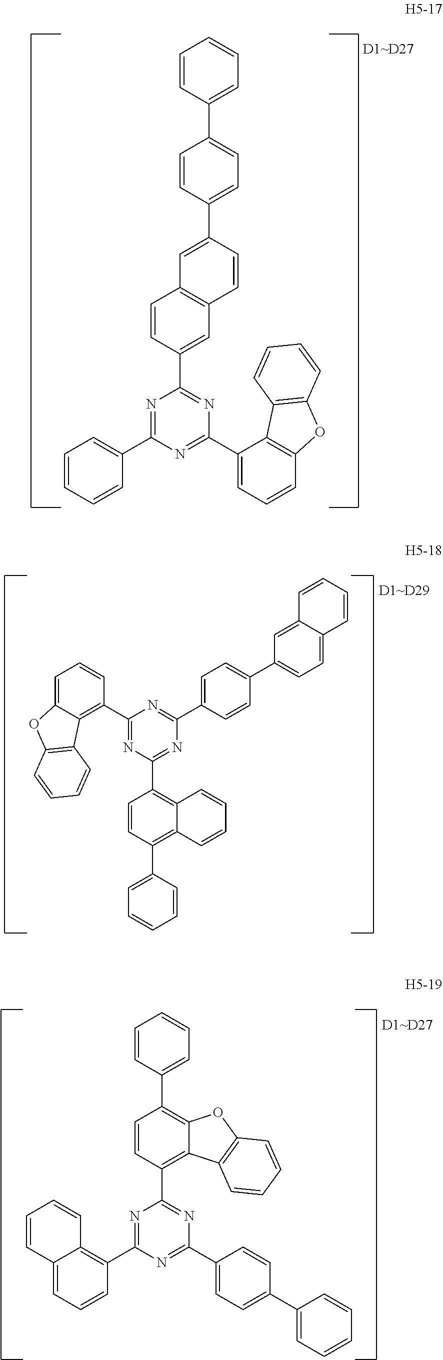

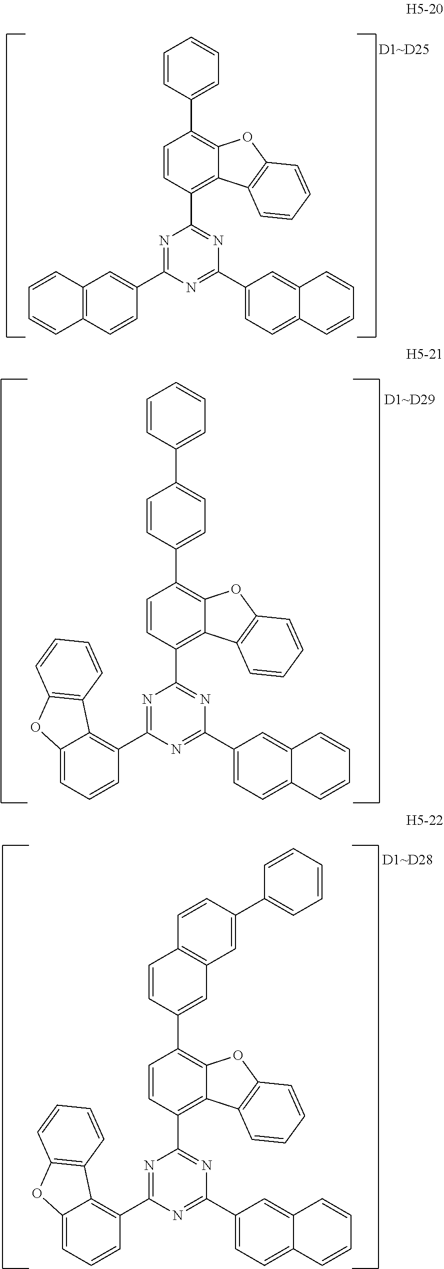

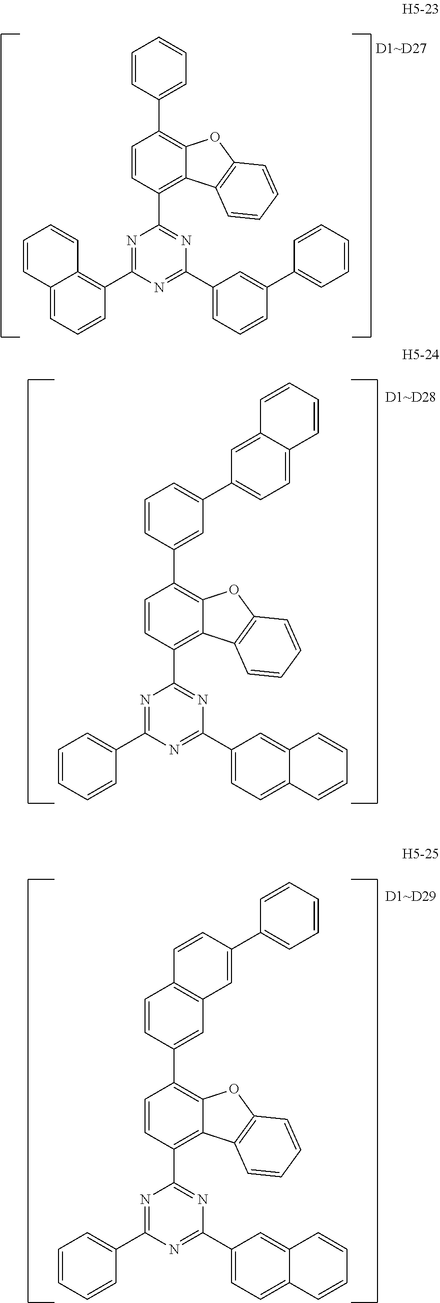

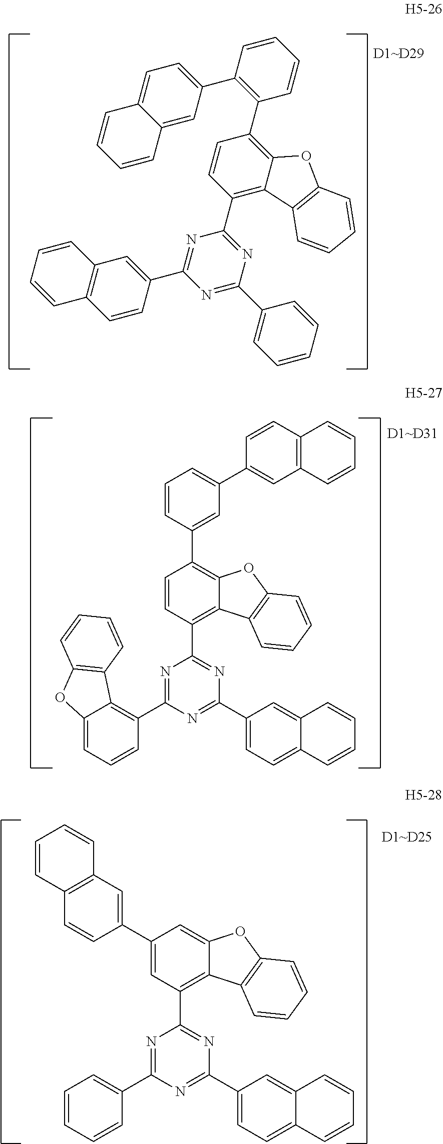

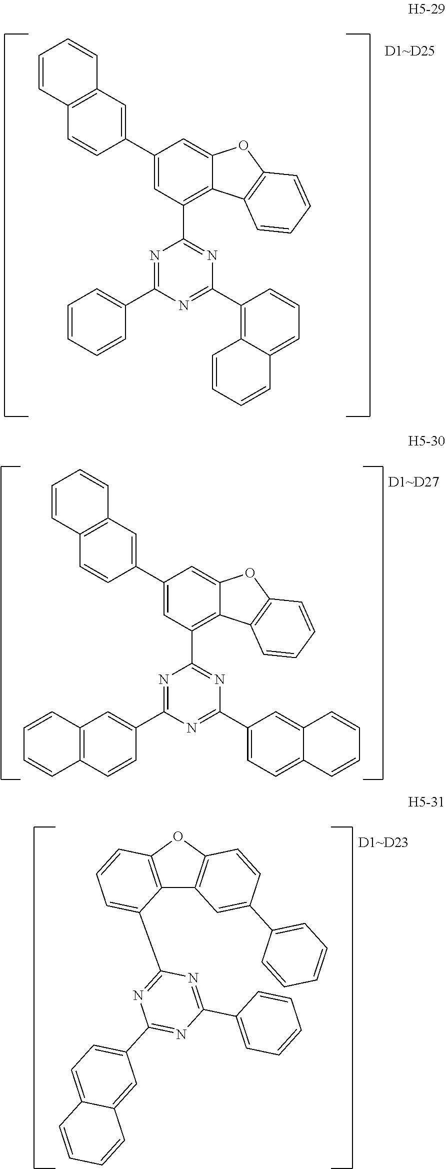

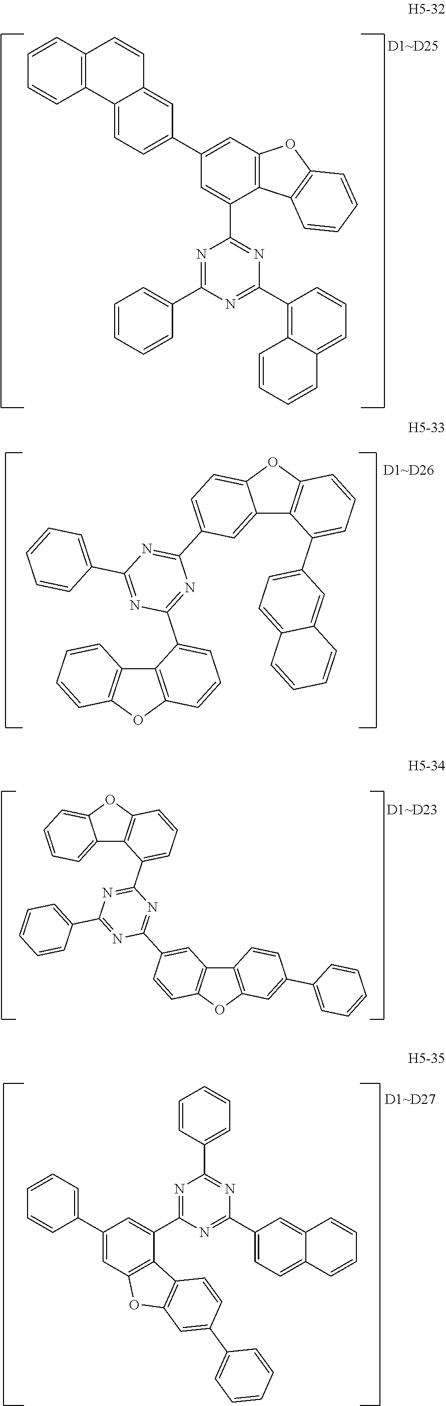

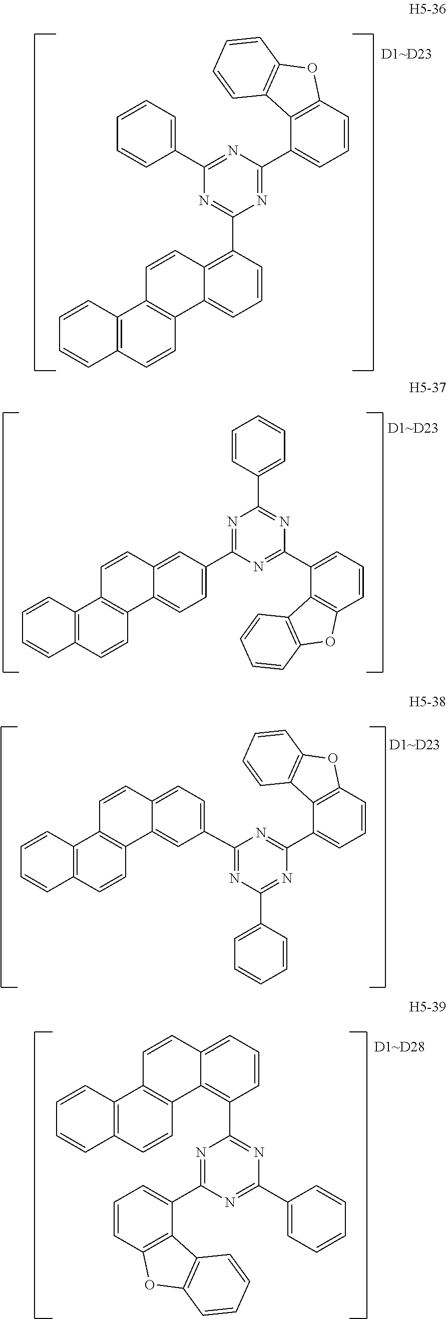

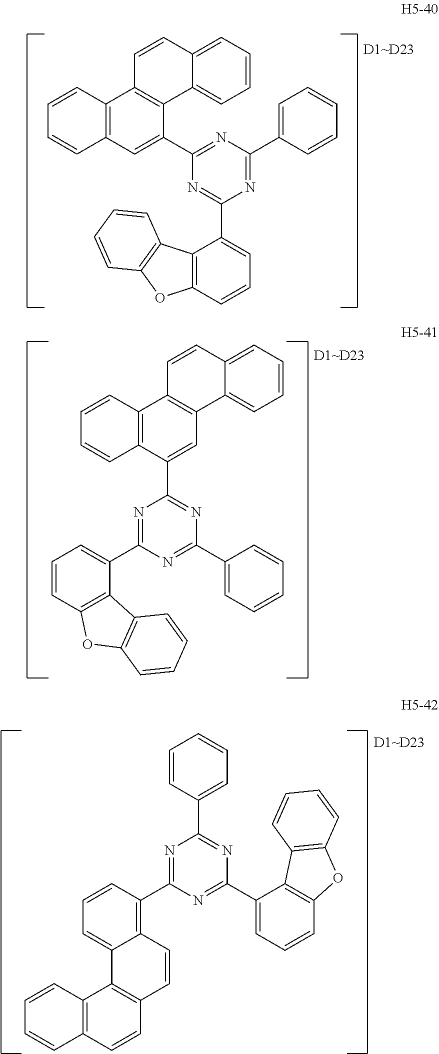

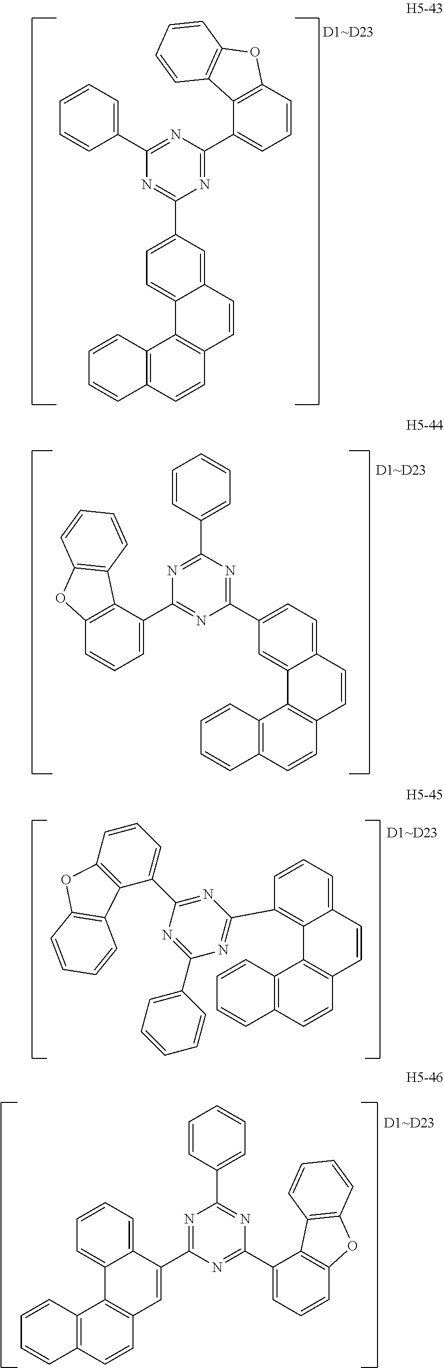

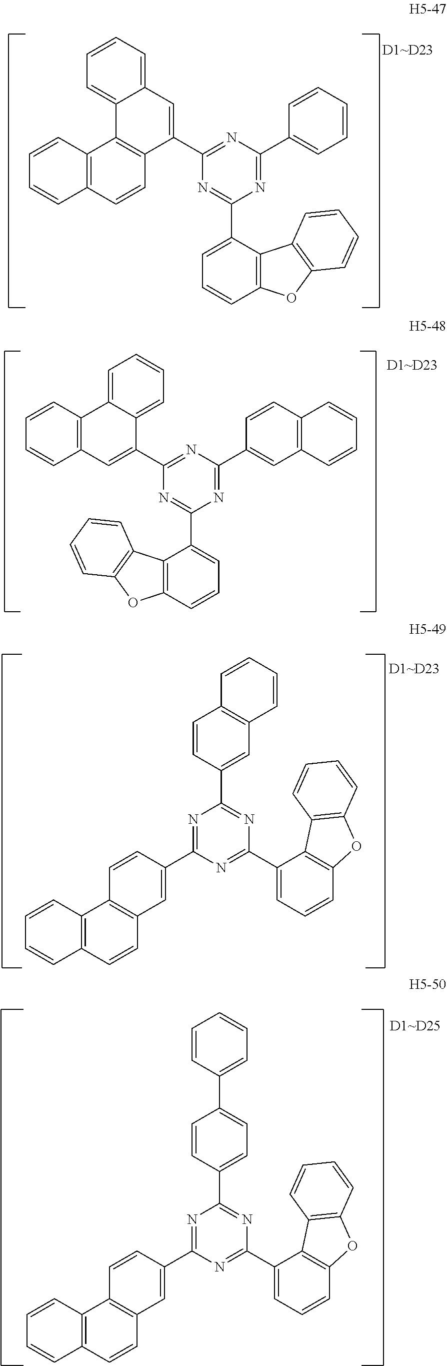

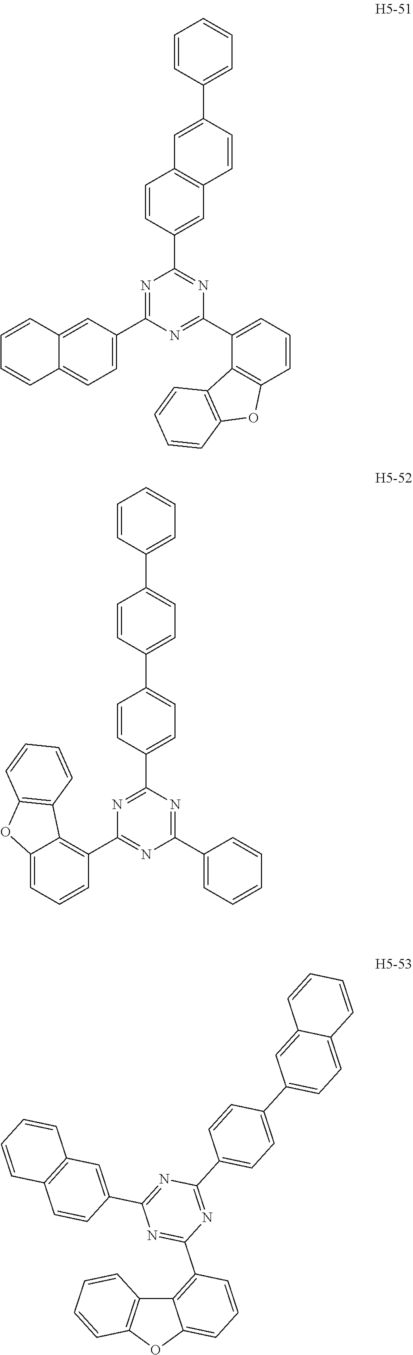 [SMILES: c1ccc(-c2ccc3cc(-c4nc(-c5ccc6ccccc6c5)nc(-c5cccc6oc7ccccc7c56)n4)ccc3c2)cc1 ; c1ccc(-c2ccc(-c3ccc(-c4nc(-c5ccccc5)nc(-c5cccc6oc7ccccc7c56)n4)cc3)cc2)cc1 ; c1ccc2cc(-c3ccc(-c4nc(-c5ccc6ccccc6c5)nc(-c5cccc6oc7ccccc7c56)n4)cc3)ccc2c1]

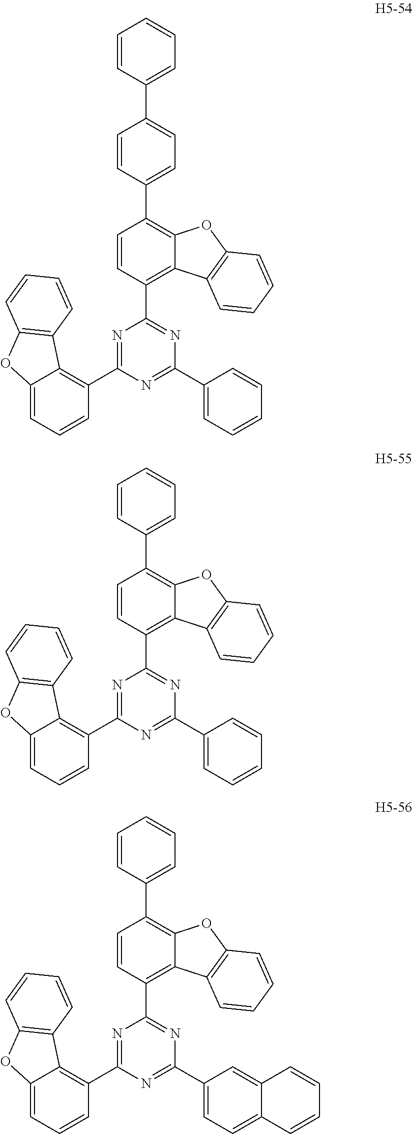 [SMILES: c1ccc(-c2ccc(-c3ccc(-c4nc(-c5ccccc5)nc(-c5cccc6oc7ccccc7c56)n4)c4c3oc3ccccc34)cc2)cc1 ; c1ccc(-c2nc(-c3cccc4oc5ccccc5c34)nc(-c3ccc(-c4ccccc4)c4oc5ccccc5c34)n2)cc1 ; c1ccc(-c2ccc(-c3nc(-c4ccc5ccccc5c4)nc(-c4cccc5oc6ccccc6c45)n3)c3c2oc2ccccc23)cc1]

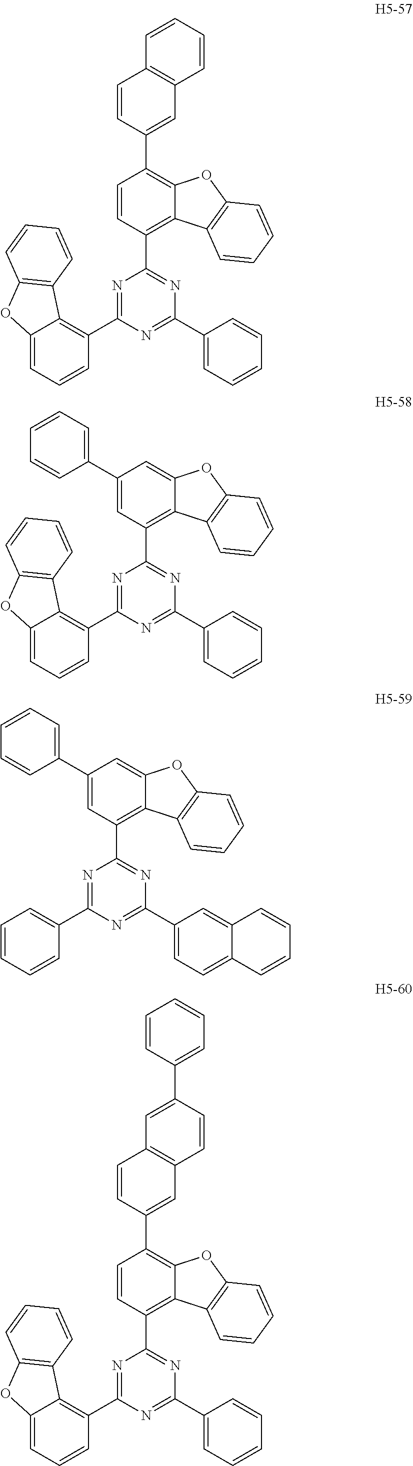

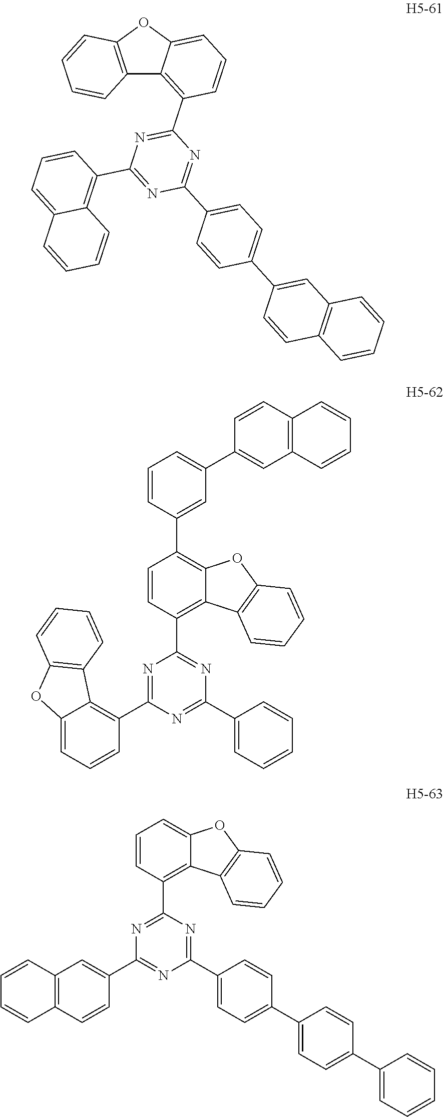

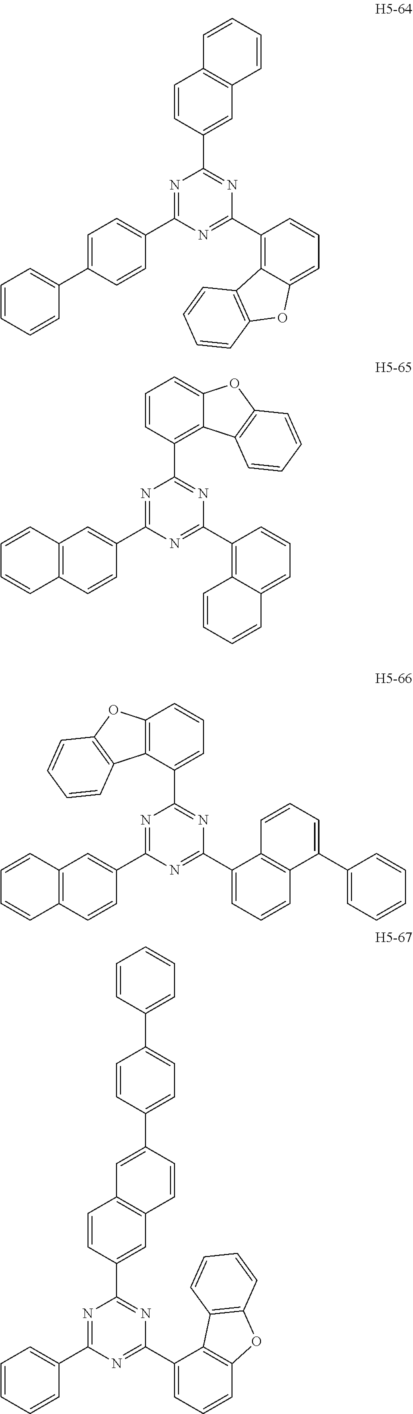

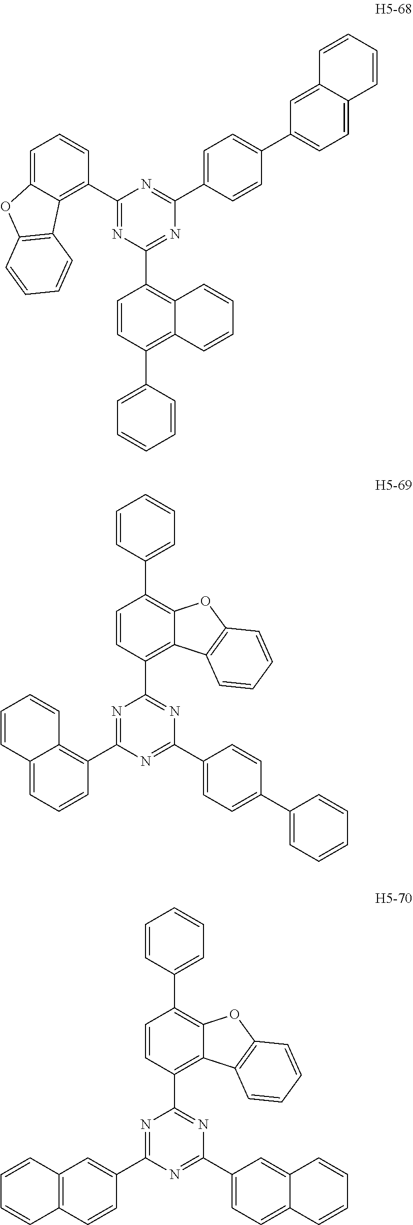

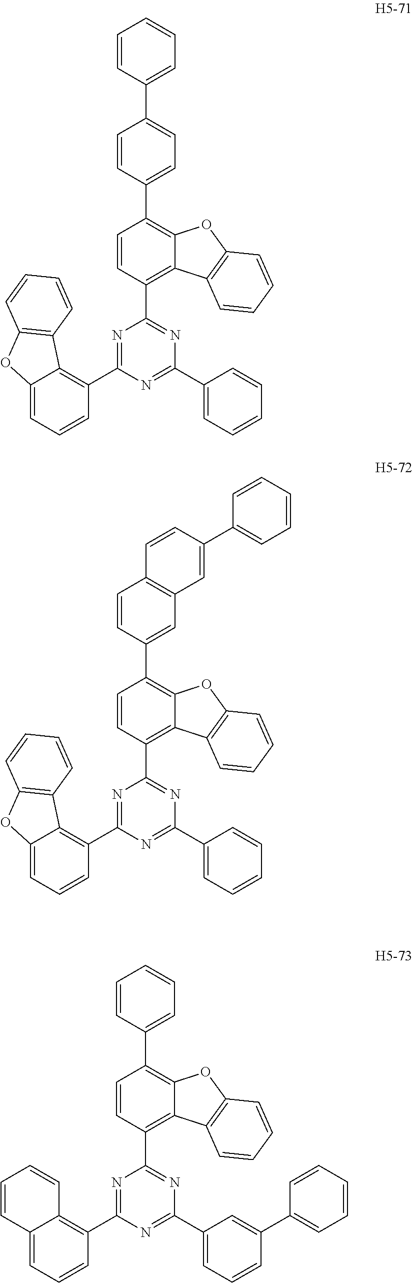 [SMILES: c1ccc(-c2ccc(-c3ccc(-c4nc(-c5ccccc5)nc(-c5cccc6oc7ccccc7c56)n4)c4c3oc3ccccc34)cc2)cc1 ; c1ccc(-c2ccc3ccc(-c4ccc(-c5nc(-c6ccccc6)nc(-c6cccc7oc8ccccc8c67)n5)c5c4oc4ccccc45)cc3c2)cc1 ; c1ccc(-c2cccc(-c3nc(-c4cccc5ccccc45)nc(-c4ccc(-c5ccccc5)c5oc6ccccc6c45)n3)c2)cc1]

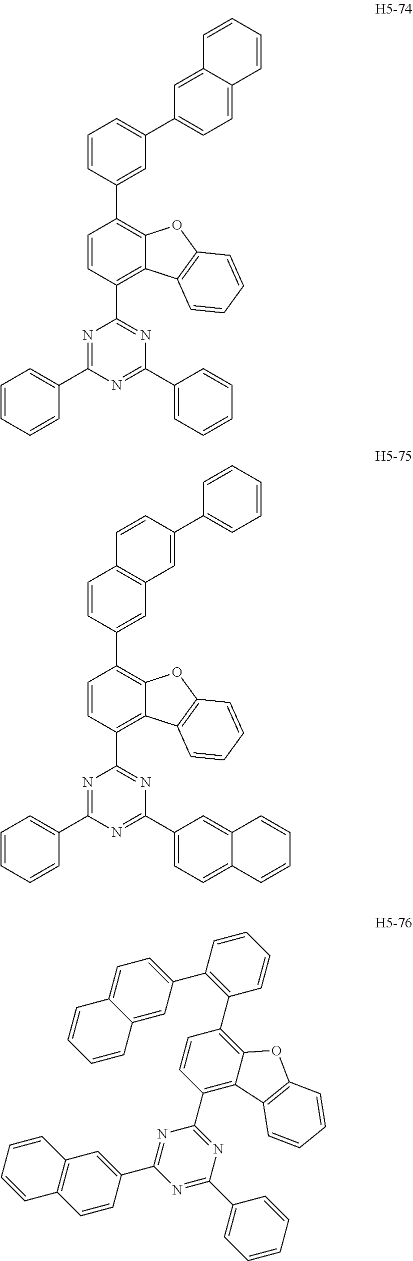

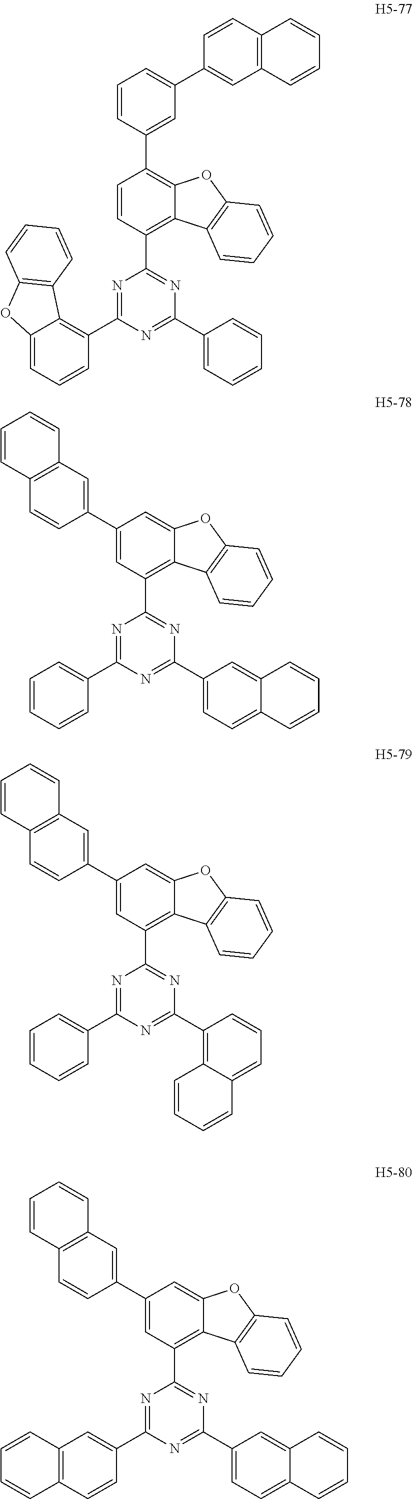

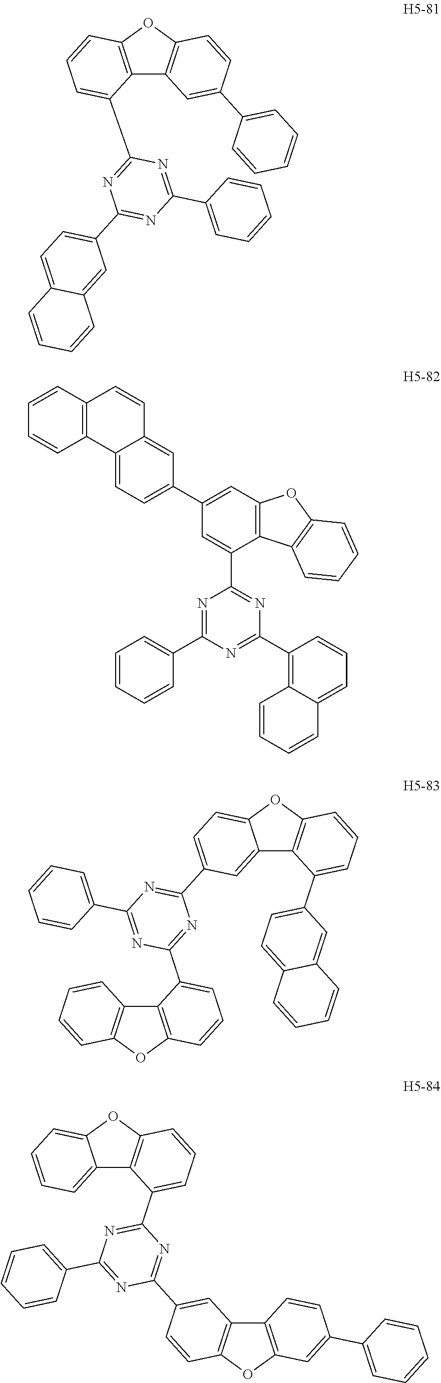

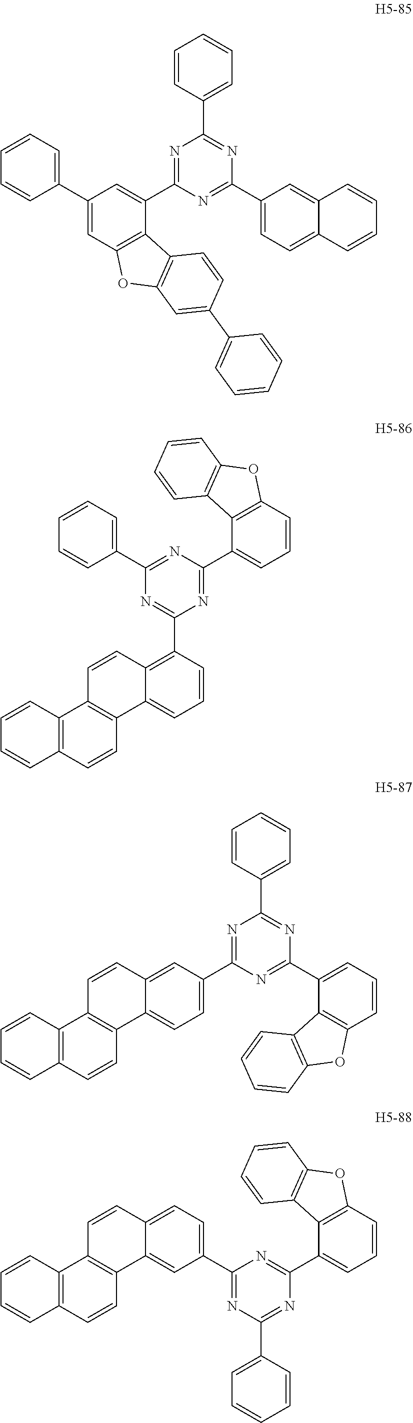 [SMILES: c1ccc(-c2ccc3c(c2)oc2cc(-c4ccccc4)cc(-c4nc(-c5ccccc5)nc(-c5ccc6ccccc6c5)n4)c23)cc1 ; c1ccc(-c2nc(-c3cccc4c3ccc3c5ccccc5ccc43)nc(-c3cccc4oc5ccccc5c34)n2)cc1 ; c1ccc(-c2nc(-c3ccc4c(ccc5c6ccccc6ccc45)c3)nc(-c3cccc4oc5ccccc5c34)n2)cc1 ; c1ccc(-c2nc(-c3ccc4ccc5c6ccccc6ccc5c4c3)nc(-c3cccc4oc5ccccc5c34)n2)cc1]

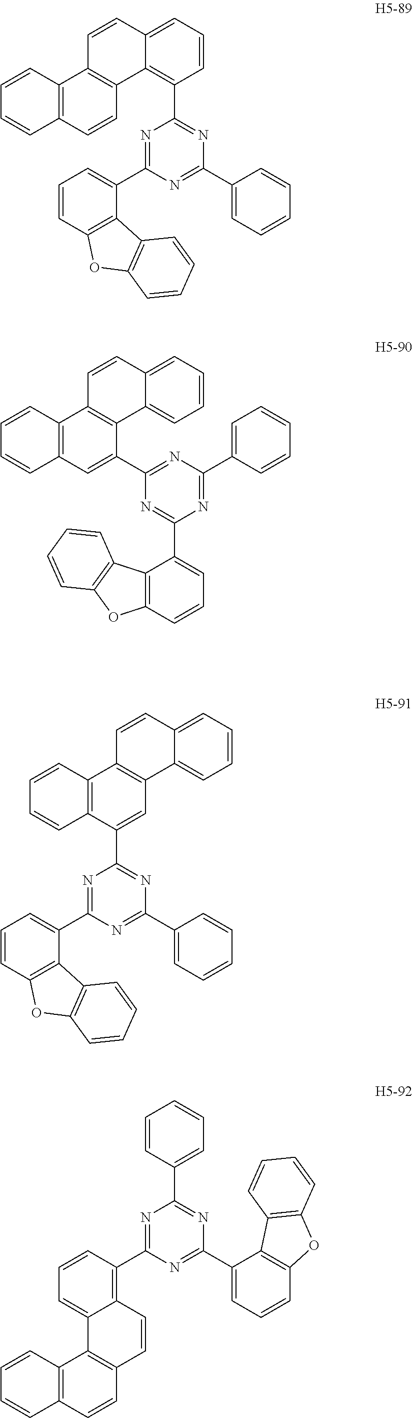 [SMILES: c1ccc(-c2nc(-c3cccc4ccc5c6ccccc6ccc5c34)nc(-c3cccc4oc5ccccc5c34)n2)cc1 ; c1ccc(-c2nc(-c3cccc4oc5ccccc5c34)nc(-c3cc4ccccc4c4ccc5ccccc5c34)n2)cc1 ; c1ccc(-c2nc(-c3cc4c5ccccc5ccc4c4ccccc34)nc(-c3cccc4oc5ccccc5c34)n2)cc1 ; c1ccc(-c2nc(-c3cccc4c3ccc3ccc5ccccc5c34)nc(-c3cccc4oc5ccccc5c34)n2)cc1]

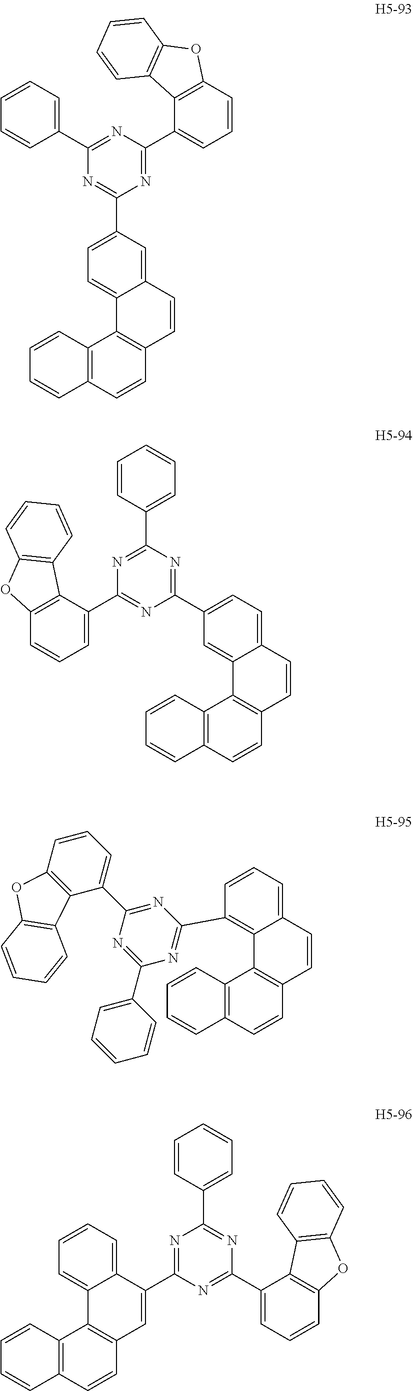 [SMILES: c1ccc(-c2nc(-c3ccc4c(ccc5ccc6ccccc6c54)c3)nc(-c3cccc4oc5ccccc5c34)n2)cc1 ; c1ccc(-c2nc(-c3ccc4ccc5ccc6ccccc6c5c4c3)nc(-c3cccc4oc5ccccc5c34)n2)cc1 ; c1ccc(-c2nc(-c3cccc4oc5ccccc5c34)nc(-c3cccc4ccc5ccc6ccccc6c5c34)n2)cc1 ; c1ccc(-c2nc(-c3cc4ccc5ccccc5c4c4ccccc34)nc(-c3cccc4oc5ccccc5c34)n2)cc1]

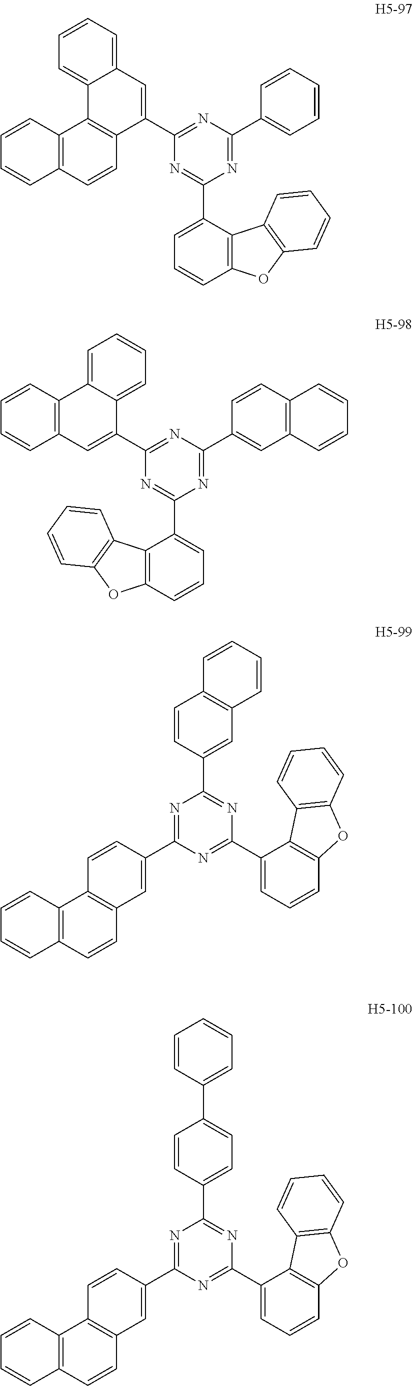 [SMILES: c1ccc(-c2nc(-c3cc4ccccc4c4c3ccc3ccccc34)nc(-c3cccc4oc5ccccc5c34)n2)cc1 ; c1ccc2cc(-c3nc(-c4cc5ccccc5c5ccccc45)nc(-c4cccc5oc6ccccc6c45)n3)ccc2c1 ; c1ccc2cc(-c3nc(-c4ccc5c(ccc6ccccc65)c4)nc(-c4cccc5oc6ccccc6c45)n3)ccc2c1 ; c1ccc(-c2ccc(-c3nc(-c4ccc5c(ccc6ccccc65)c4)nc(-c4cccc5oc6ccccc6c45)n3)cc2)cc1]

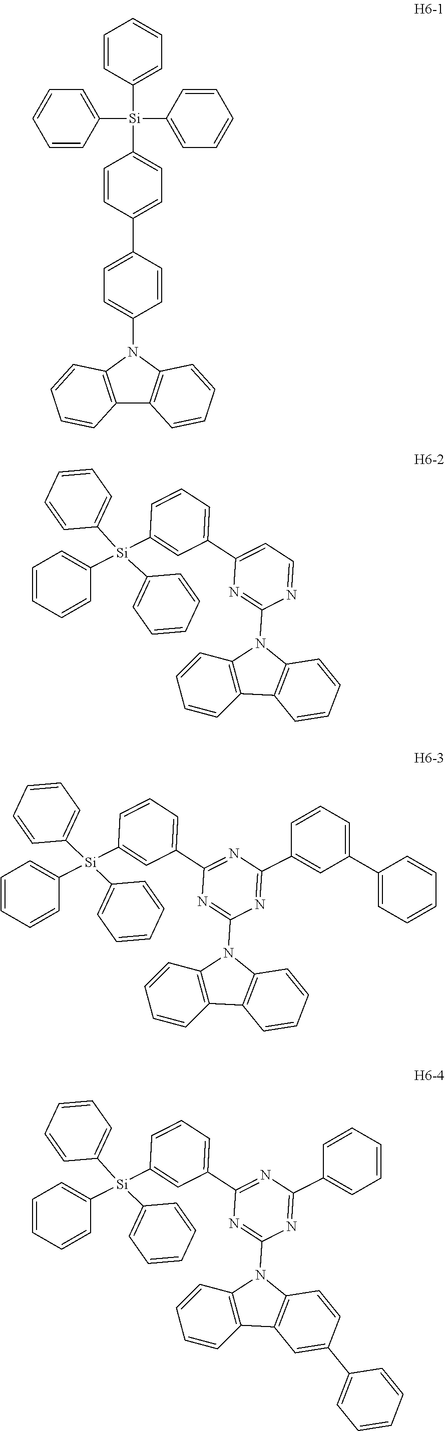

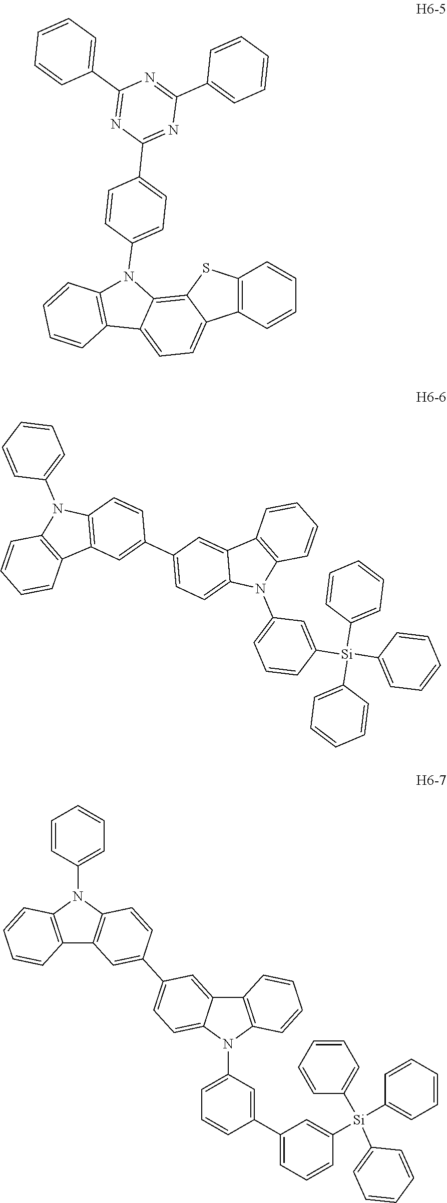 [SMILES: c1ccc(-c2nc(-c3ccccc3)nc(-c3ccc(-n4c5ccccc5c5ccc6c7ccccc7sc6c54)cc3)n2)cc1 ; c1ccc(-n2c3ccccc3c3cc(-c4ccc5c(c4)c4ccccc4n5-c4cccc([Si](c5ccccc5)(c5ccccc5)c5ccccc5)c4)ccc32)cc1 ; c1ccc(-n2c3ccccc3c3cc(-c4ccc5c(c4)c4ccccc4n5-c4cccc(-c5cccc([Si](c6ccccc6)(c6ccccc6)c6ccccc6)c5)c4)ccc32)cc1]

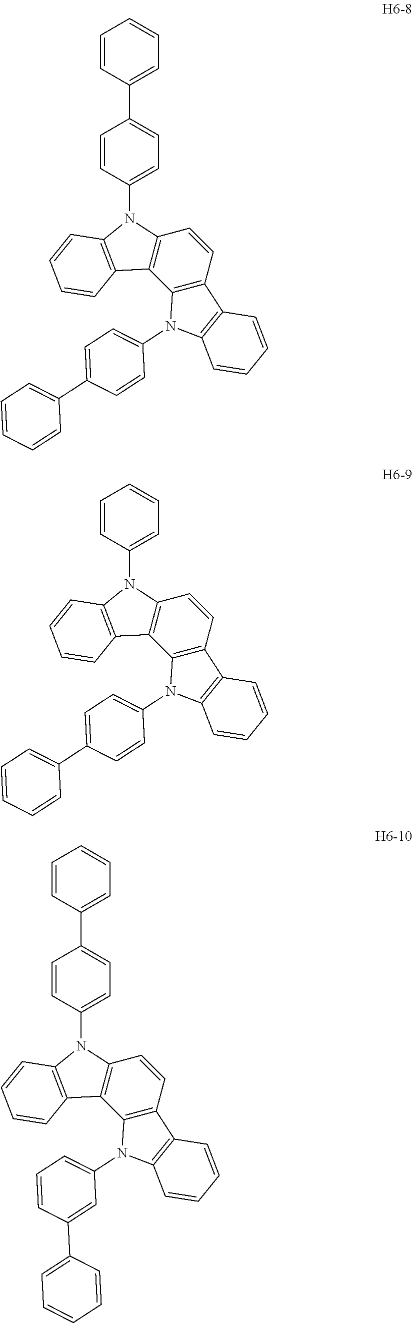 [SMILES: c1ccc(-c2ccc(-n3c4ccccc4c4c3ccc3c5ccccc5n(-c5ccc(-c6ccccc6)cc5)c34)cc2)cc1 ; c1ccc(-c2ccc(-n3c4ccccc4c4ccc5c(c6ccccc6n5-c5ccccc5)c43)cc2)cc1 ; c1ccc(-c2ccc(-n3c4ccccc4c4c3ccc3c5ccccc5n(-c5cccc(-c6ccccc6)c5)c34)cc2)cc1]

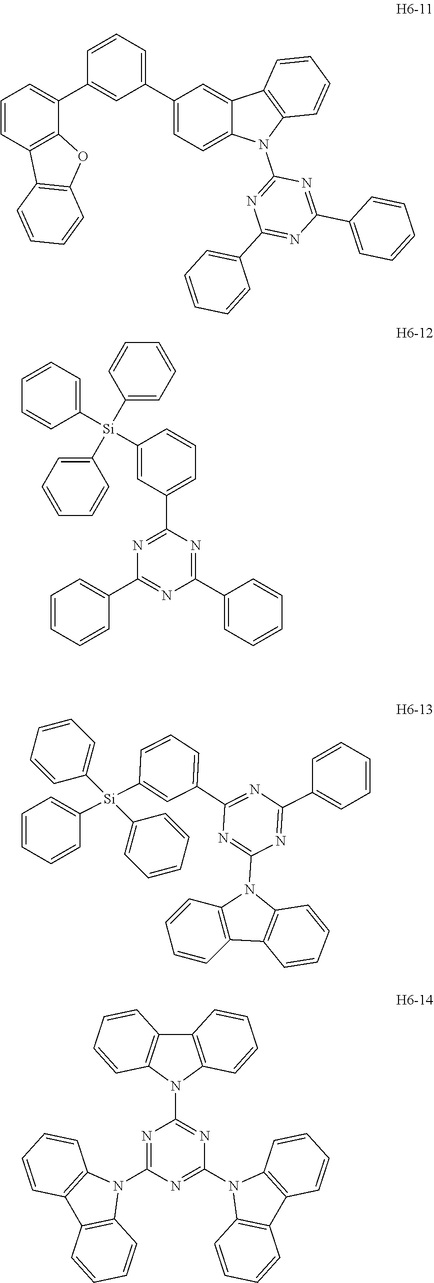 [SMILES: c1ccc(-c2nc(-c3ccccc3)nc(-n3c4ccccc4c4cc(-c5cccc(-c6cccc7c6oc6ccccc67)c5)ccc43)n2)cc1 ; c1ccc(-c2nc(-c3ccccc3)nc(-c3cccc([Si](c4ccccc4)(c4ccccc4)c4ccccc4)c3)n2)cc1 ; c1ccc(-c2nc(-c3cccc([Si](c4ccccc4)(c4ccccc4)c4ccccc4)c3)nc(-n3c4ccccc4c4ccccc43)n2)cc1 ; c1ccc2c(c1)c1ccccc1n2-c1nc(-n2c3ccccc3c3ccccc32)nc(-n2c3ccccc3c3ccccc32)n1]

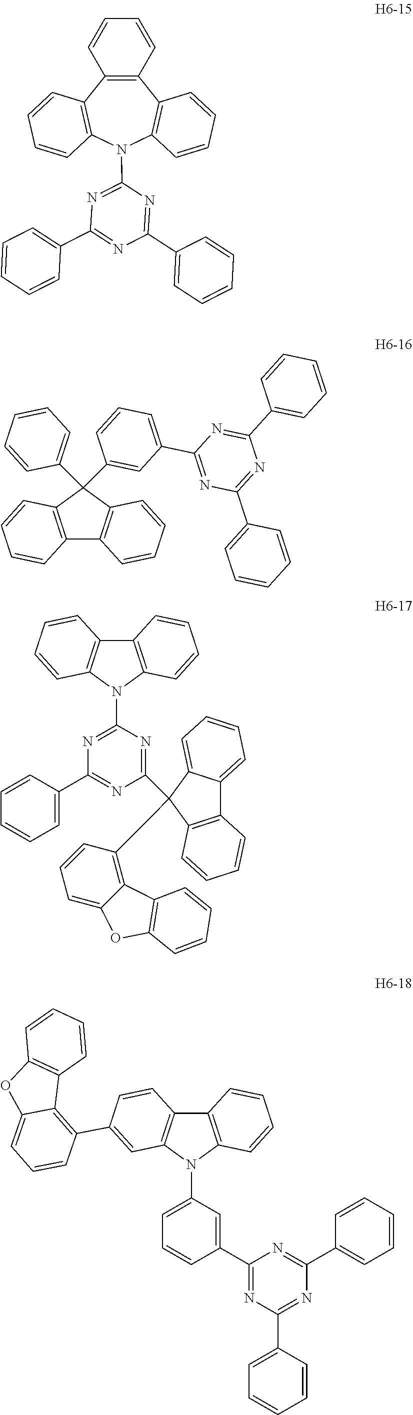 [SMILES: c1ccc(-c2nc(-c3ccccc3)nc(N3c4ccccc4-c4ccccc4-c4ccccc43)n2)cc1 ; c1ccc(-c2nc(-c3ccccc3)nc(-c3cccc(C4(c5ccccc5)c5ccccc5-c5ccccc54)c3)n2)cc1 ; c1ccc(-c2nc(-n3c4ccccc4c4ccccc43)nc(C3(c4cccc5oc6ccccc6c45)c4ccccc4-c4ccccc43)n2)cc1 ; c1ccc(-c2nc(-c3ccccc3)nc(-c3cccc(-n4c5ccccc5c5ccc(-c6cccc7oc8ccccc8c67)cc54)c3)n2)cc1]

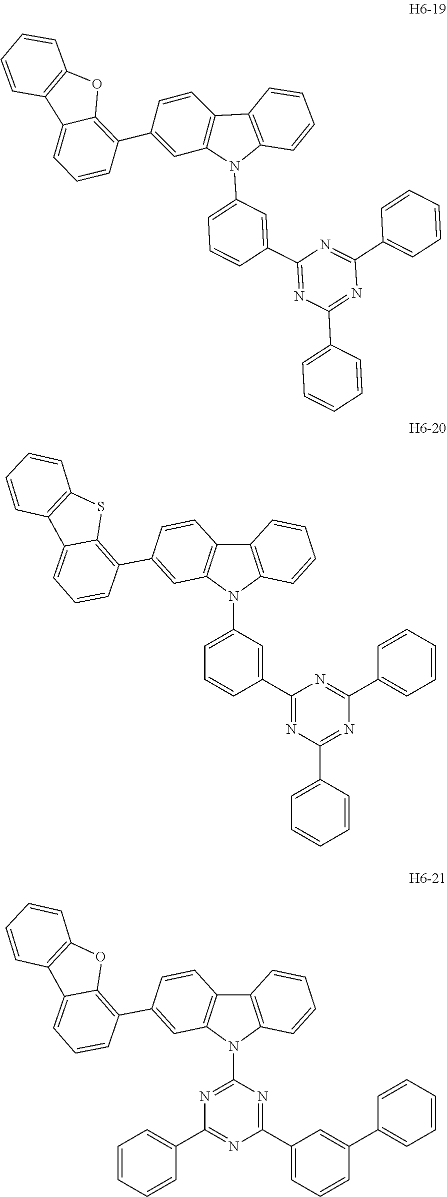 [SMILES: c1ccc(-c2nc(-c3ccccc3)nc(-c3cccc(-n4c5ccccc5c5ccc(-c6cccc7c6oc6ccccc67)cc54)c3)n2)cc1 ; c1ccc(-c2nc(-c3ccccc3)nc(-c3cccc(-n4c5ccccc5c5ccc(-c6cccc7c6sc6ccccc67)cc54)c3)n2)cc1 ; c1ccc(-c2cccc(-c3nc(-c4ccccc4)nc(-n4c5ccccc5c5ccc(-c6cccc7c6oc6ccccc67)cc54)n3)c2)cc1]

The charge generation layer according to one embodiment includes an organic electroluminescent compound represented by the following Formula 2.

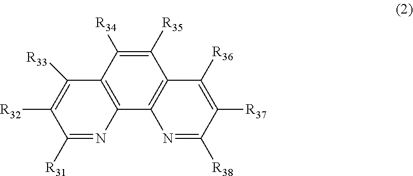

In Formula 2,
R₃₁ to R₃₆ each independently represent, hydrogen, deuterium, halogen, cyano, a substituted or unsubstituted (C1-C30)alkyl, a substituted or unsubstituted (C2-C30)alkenyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring of (C3-C30) aliphatic ring and (C6-C30) aromatic ring, a substituted or unsubstituted mono- or di-(C1-C30)alkylamino, a substituted or unsubstituted mono- or di-(C2-C30)alkenylamino, a substituted or unsubstituted mono- or di-(C6-C30)arylamino, a substituted or unsubstituted mono- or di-(3- to 30-membered)heteroarylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino; or may be linked to the adjacent substituents to form a ring(s);
provided that at least one of R₃₁ to R₃₅ is -(L₃)_{c}-(HAr)_{d};
L₃ represents a single bond, a substituted or unsubstituted (C6-C30)arylene, or a substituted or unsubstituted (3- to 30-membered)heteroarylene;
HAr represents a substituted or unsubstituted (C6-C30)aryl or a substituted or unsubstituted (3- to 30-membered)heteroaryl; and
c is an integer of 1 to 3, and d is an integer of 1 or 2;
when c and d are 2 or more, each of L₃ and each of HAr may be the same or different;
provided that if it contains at least one deuterium in Formula 2, the compound where L₃ and HAr contain anthracene is excluded.

The compound represented by Formula 2 according to one embodiment may be included in the N-type charge generation layer. In the compound of Formula 2 according to the present disclosure, the phenanthroline moiety includes a sp2 hybrid orbital nitrogen that is relatively rich in electrons. In particular, the phenanthroline moiety has a structure in which two nitrogens are next to each other, and thus it can covalently bond with surrounding hydrogen or coordinate with alkaline metals or alkaline earth metals such as Li and Yb. When applying the organic electroluminescent compound of Formula 2 having such a phenanthroline moiety to an N-type charge generation layer, the phenanthroline moiety can improve electron injection and transfer capabilities by trapping doped alkali metals or alkaline earth metals and increasing the electron density within the molecule. In addition, when the compound of Formula 2 is applied to the N-type charge generation layer of an organic electroluminescent device, the nitrogen of the phenanthroline moiety can form a gap state by binding with an alkali metal or alkaline earth metal, which is a dopant of the N-type charge generation layer.

In one embodiment, L may be a single bond, a substituted or unsubstituted (C6-C30)arylene, or a substituted or unsubstituted (5- to 30-membered)heteroarylene, preferably a single bond, a substituted or unsubstituted (C6-C25)arylene, or a substituted or unsubstituted (5- to 25-membered)heteroarylene, more preferably a single bond, a substituted or unsubstituted (C6-C18)arylene, or a substituted or unsubstituted (5- to 18-membered)heteroarylene. For example, L₃ may be a single bond, phenylene unsubstituted or substituted with phenyl or phenanthroline, a substituted or unsubstituted naphthylene, a substituted or unsubstituted anthracenylene, a substituted or unsubstituted pyridylene, or pyrimidylene unsubstituted or substituted with phenyl.

In one embodiment, HAr may be a substituted or unsubstituted (C6-C30)aryl or a substituted or unsubstituted (5- to 30-membered)heteroaryl, preferably a substituted or unsubstituted (C6-C25)aryl or a substituted or unsubstituted (5- to 25-membered)heteroaryl, more preferably a substituted or unsubstituted (C6-C18)aryl or a substituted or unsubstituted nitrogen-containing (5- to 25-membered)heteroaryl. For example, HAr may be a substituted or unsubstituted pyridyl, a substituted or unsubstituted pyrazinyl, pyrimidyl unsubstituted or substituted with pyridyl, triazinyl unsubstituted or substituted with at least one phenyl, a substituted or unsubstituted isoquinolinyl, a substituted or unsubstituted quinolinyl, a substituted or unsubstituted quinazolyl, a substituted or unsubstituted quinoxalinyl, a substituted or unsubstituted phenanthrolinyl, a substituted or unsubstituted pyridoindolyl, phenyl unsubstituted or substituted with pyrenyl, fluorenyl unsubstituted or substituted with diethyl, a substituted or unsubstituted phenanthrenyl, or a substituted or unsubstituted anthracenyl. For example, the substituted group may be substituted with phenyl unsubstituted or substituted with phenanthroline or diphenylphosphine oxide, naphthyl unsubstituted or substituted with phenyl, o-biphenyl, m-biphenyl, p-biphenyl, dimethylfluorenyl, diphenylfluorenyl, pyridyl, dibenzofuranyl, or dibenzothiophenyl, etc.

In one embodiment, at least one of R₃₁ to R₃₈ may be -(L₃)_{c}-(HAr)_{d}, preferably at least one of R₃₁ and R₃₈ may be -(L)_{c}-(HAr)_{d}, and R₃₁ or R₃₈ which does not represent -(L₃)_{c}-(HAr)_{d} and R₃₂ to R₃₇ each independently may be, hydrogen, deuterium, a substituted or unsubstituted (C1-C30)alkyl, a substituted or unsubstituted (C6-C30)aryl; or may be linked to the adjacent substituents to form a ring(s). For example, R₃₈ may be hydrogen, deuterium, methyl, methyl-d3, tert-butyl, phenyl unsubstituted or substituted with naphthyl; dibenzothiophenyl; or dibenzofuranyl, a substituted or unsubstituted naphthyl, a substituted or unsubstituted o-biphenyl, a substituted or unsubstituted m-biphenyl, a substituted or unsubstituted p-biphenyl, a substituted or unsubstituted o-terphenyl, a substituted or unsubstituted m-terphenyl, fluorenyl unsubstituted or substituted with at least one of methyl and phenyl, a substituted or unsubstituted anthracenyl, a substituted or unsubstituted phenanthrenyl, a substituted or unsubstituted triphenylenyl, a substituted or unsubstituted pyrenyl, pyridyl unsubstituted or substituted with a substituted or unsubstituted phenyl, a substituted or unsubstituted dibenzofuranyl, or a substituted or unsubstituted dibenzothiophenyl. For example, R₃₄ and R₃₅ each independently may be hydrogen, deuterium, or methyl; or may be linked to each other to form a benzene ring fused with phenanthroline. For example, R₃₂ and R₃₃ each independently may be hydrogen, deuterium, phenyl, or m-biphenyl. For example, R₃₆ and R₃₇ each independently may be, hydrogen, deuterium, or phenyl; or may be linked to each other to form a benzene ring fused with phenanthroline.

According to one embodiment, if one compound represented by Formula 2 comprises deuterium, the compound comprises at least one deuterium, preferably at least one of R₃₁ to R₃₈ may comprise deuterium, more preferably each of R₃₁ to R₃₈, and -(L₃)_{c}-(HAr)_{d} may comprise at least one deuterium. According to one embodiment, if one compound represented by Formula 2 comprises deuterium, the degree of deuteriumization is preferably at least 30% of the total hydrogen, more preferably at least 40% of the total hydrogen, even more preferably at least 50% of the total hydrogen. When deuterated the compound of Formula 2 with a number equal to or higher than the lower limit, the bond dissociation energy according to deuteration increases, thereby increasing the stability of the compound. When such a compound is used in an organic electroluminescent device, improved lifespan characteristics may be exhibited.

According to one embodiment, when the compound represented by Formula 2 is deuterated, the compounds in which L₃ represents a substituted or unsubstituted anthracenylene and/or HAr represents a substituted or unsubstituted anthracenyl are excluded.

According to one embodiment, the compound represented by Formula 2 may be more specifically illustrated by the following compounds, but is not limited thereto.

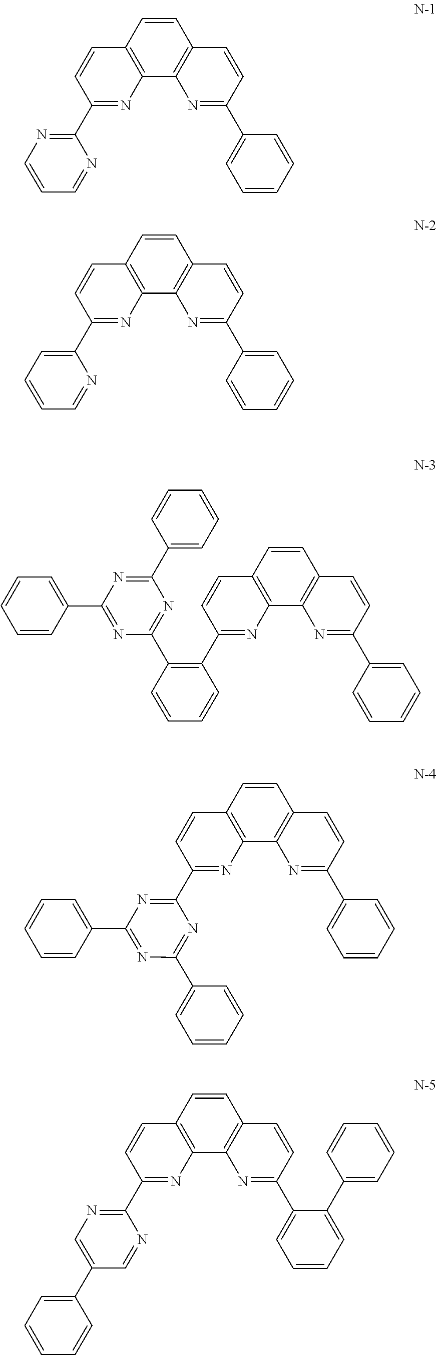 [SMILES: c1ccc(-c2ccc3ccc4ccc(-c5ncccn5)nc4c3n2)cc1 ; c1ccc(-c2ccc3ccc4ccc(-c5ccccn5)nc4c3n2)cc1 ; c1ccc(-c2ccc3ccc4ccc(-c5ccccc5-c5nc(-c6ccccc6)nc(-c6ccccc6)n5)nc4c3n2)cc1 ; c1ccc(-c2ccc3ccc4ccc(-c5nc(-c6ccccc6)nc(-c6ccccc6)n5)nc4c3n2)cc1 ; c1ccc(-c2cnc(-c3ccc4ccc5ccc(-c6ccccc6-c6ccccc6)nc5c4n3)nc2)cc1]

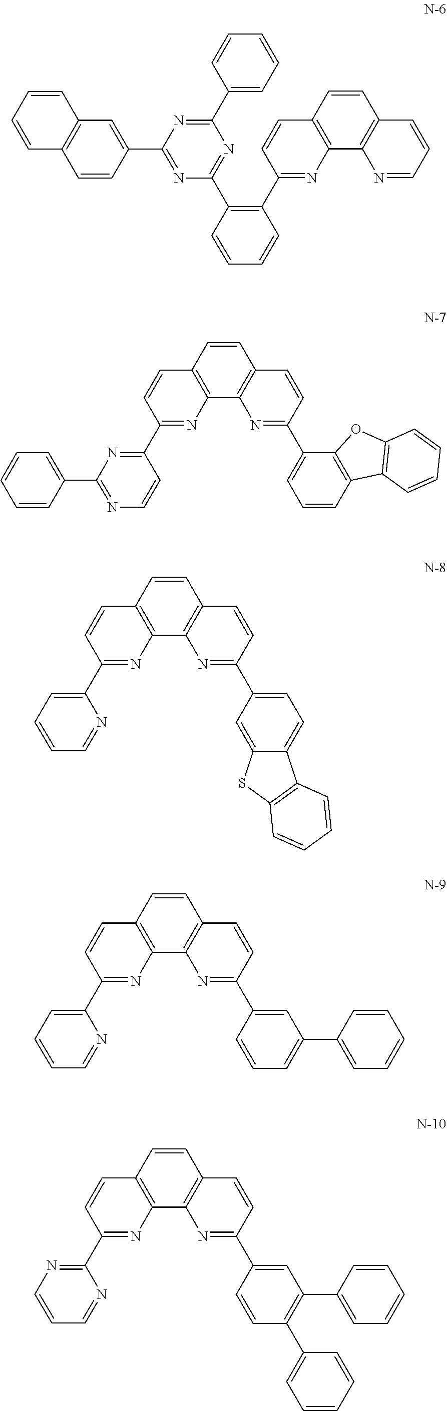 [SMILES: c1ccc(-c2nc(-c3ccc4ccccc4c3)nc(-c3ccccc3-c3ccc4ccc5cccnc5c4n3)n2)cc1 ; c1ccc(-c2nccc(-c3ccc4ccc5ccc(-c6cccc7c6oc6ccccc67)nc5c4n3)n2)cc1 ; c1ccc(-c2ccc3ccc4ccc(-c5ccc6c(c5)sc5ccccc56)nc4c3n2)nc1 ; c1ccc(-c2cccc(-c3ccc4ccc5ccc(-c6ccccn6)nc5c4n3)c2)cc1 ; c1ccc(-c2ccc(-c3ccc4ccc5ccc(-c6ncccn6)nc5c4n3)cc2-c2ccccc2)cc1]

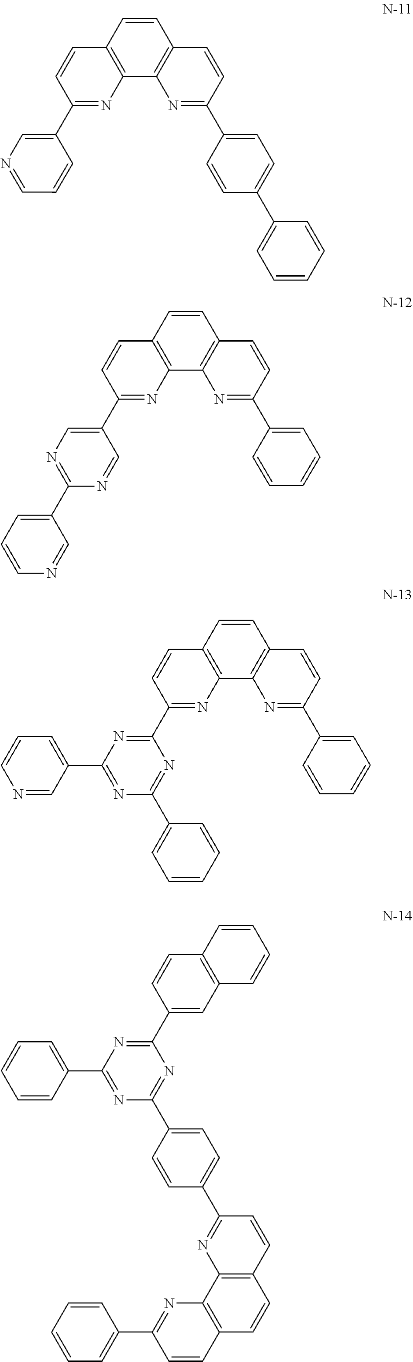

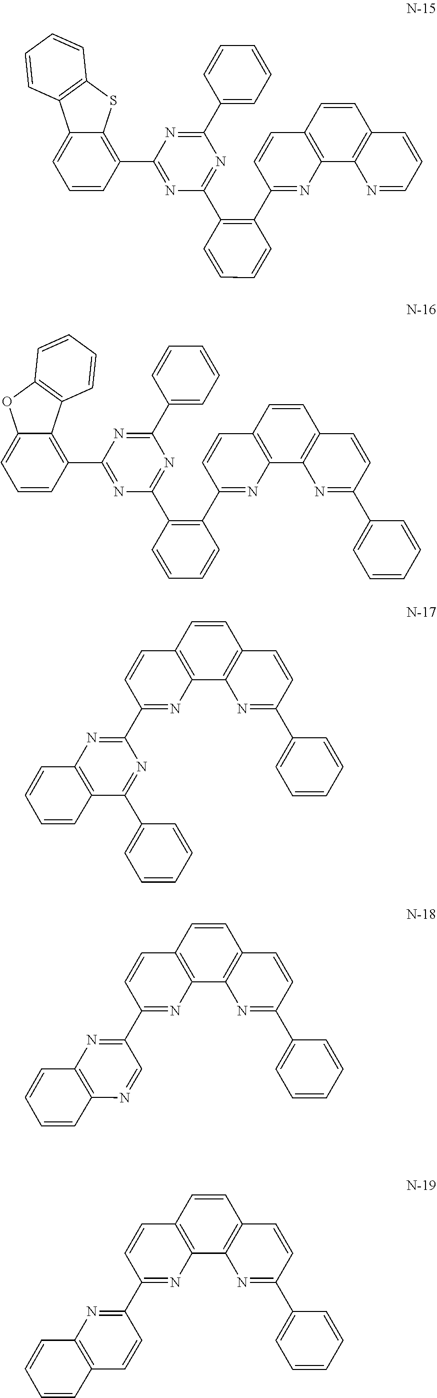

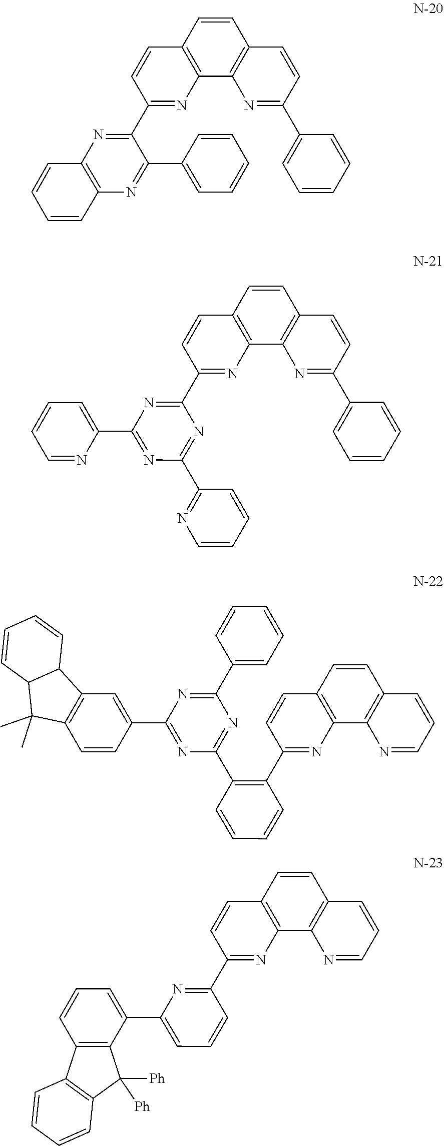

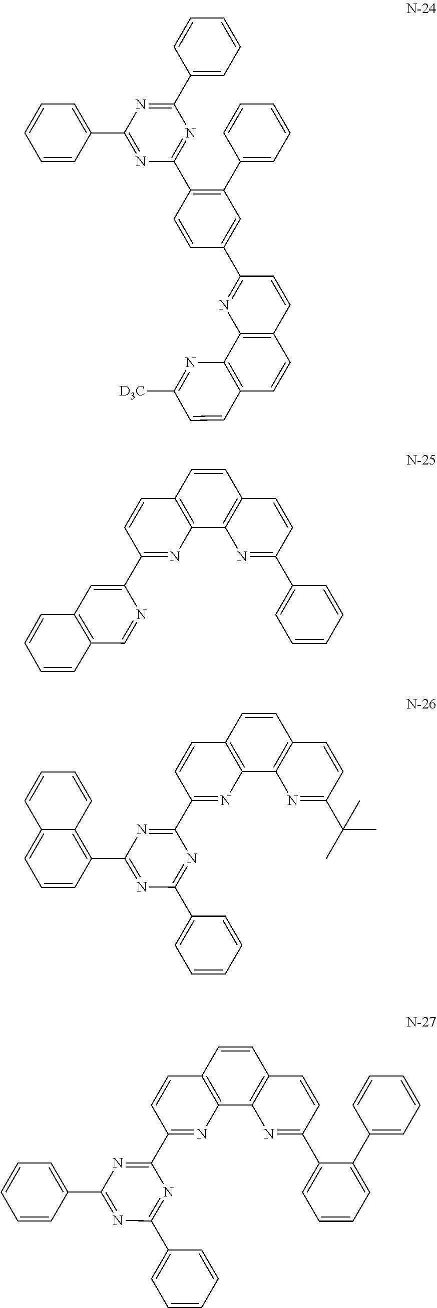 [SMILES: [2H]C([2H])([2H])c1ccc2ccc3ccc(-c4ccc(-c5nc(-c6ccccc6)nc(-c6ccccc6)n5)c(-c5ccccc5)c4)nc3c2n1 ; c1ccc(-c2ccc3ccc4ccc(-c5cc6ccccc6cn5)nc4c3n2)cc1 ; CC(C)(C)c1ccc2ccc3ccc(-c4nc(-c5ccccc5)nc(-c5cccc6ccccc56)n4)nc3c2n1 ; c1ccc(-c2nc(-c3ccccc3)nc(-c3ccc4ccc5ccc(-c6ccccc6-c6ccccc6)nc5c4n3)n2)cc1]

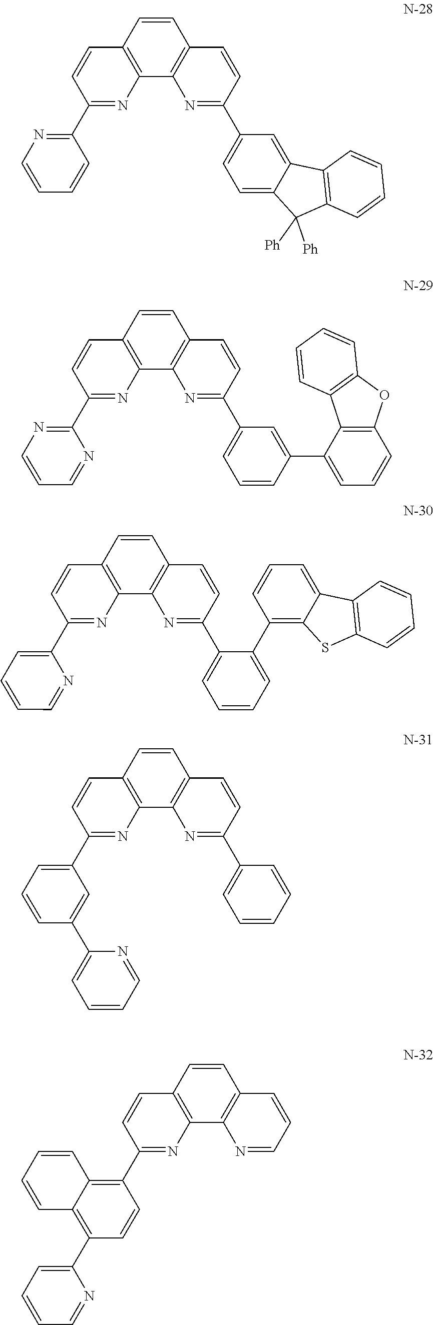 [SMILES: c1ccc(C2(c3ccccc3)c3ccccc3-c3cc(-c4ccc5ccc6ccc(-c7ccccn7)nc6c5n4)ccc32)cc1 ; c1cnc(-c2ccc3ccc4ccc(-c5cccc(-c6cccc7oc8ccccc8c67)c5)nc4c3n2)nc1 ; c1ccc(-c2ccc3ccc4ccc(-c5ccccc5-c5cccc6c5sc5ccccc56)nc4c3n2)nc1 ; c1ccc(-c2ccc3ccc4ccc(-c5cccc(-c6ccccn6)c5)nc4c3n2)cc1 ; c1ccc(-c2ccc(-c3ccc4ccc5cccnc5c4n3)c3ccccc23)nc1]

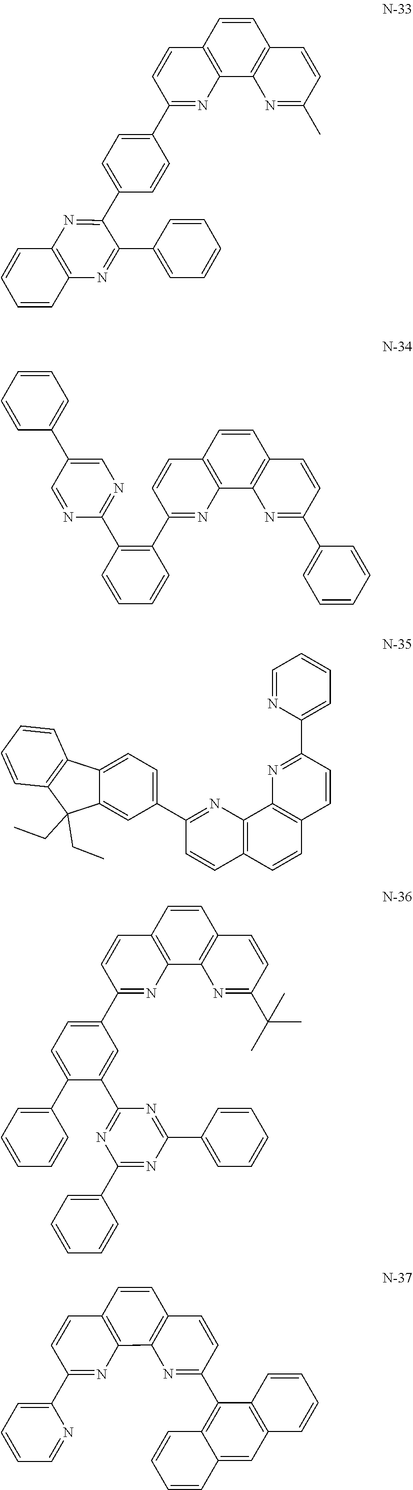

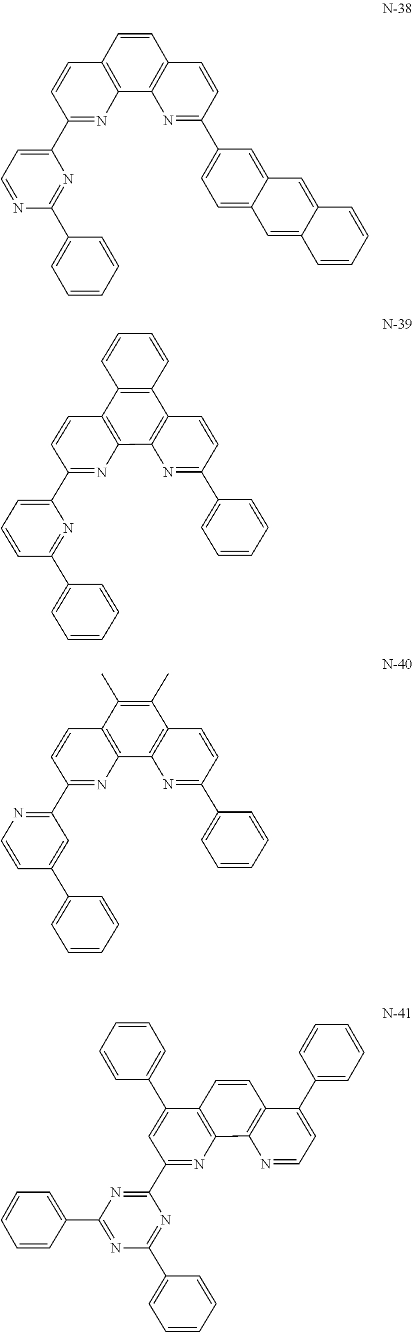

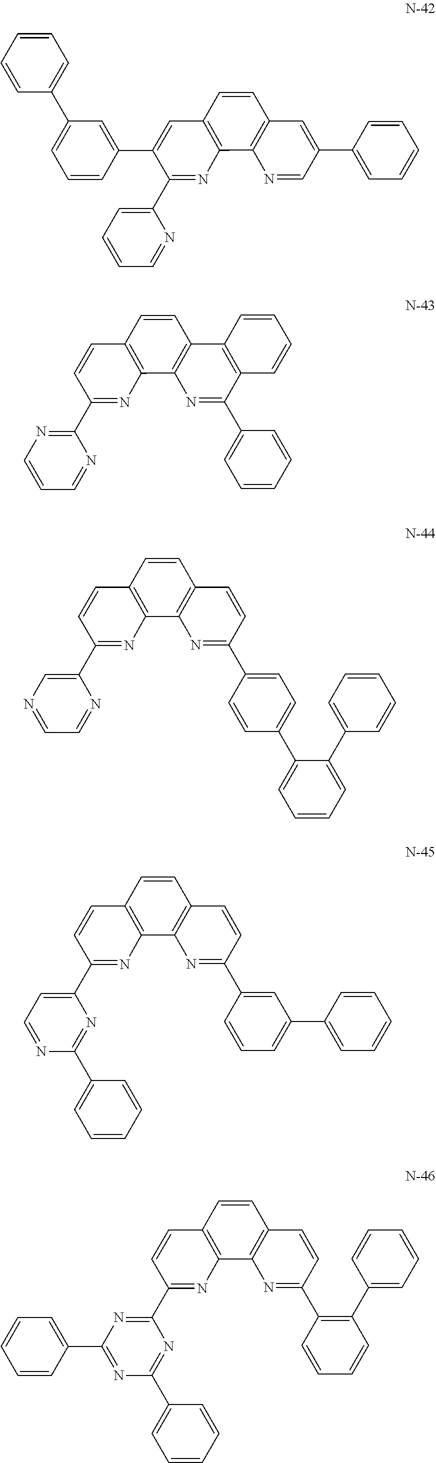 [SMILES: c1ccc(-c2cccc(-c3cc4ccc5cc(-c6ccccc6)cnc5c4nc3-c3ccccn3)c2)cc1 ; c1ccc(-c2nc3c(ccc4ccc(-c5ncccn5)nc43)c3ccccc23)cc1 ; c1ccc(-c2ccccc2-c2ccc(-c3ccc4ccc5ccc(-c6cnccn6)nc5c4n3)cc2)cc1 ; c1ccc(-c2cccc(-c3ccc4ccc5ccc(-c6ccnc(-c7ccccc7)n6)nc5c4n3)c2)cc1 ; c1ccc(-c2nc(-c3ccccc3)nc(-c3ccc4ccc5ccc(-c6ccccc6-c6ccccc6)nc5c4n3)n2)cc1]

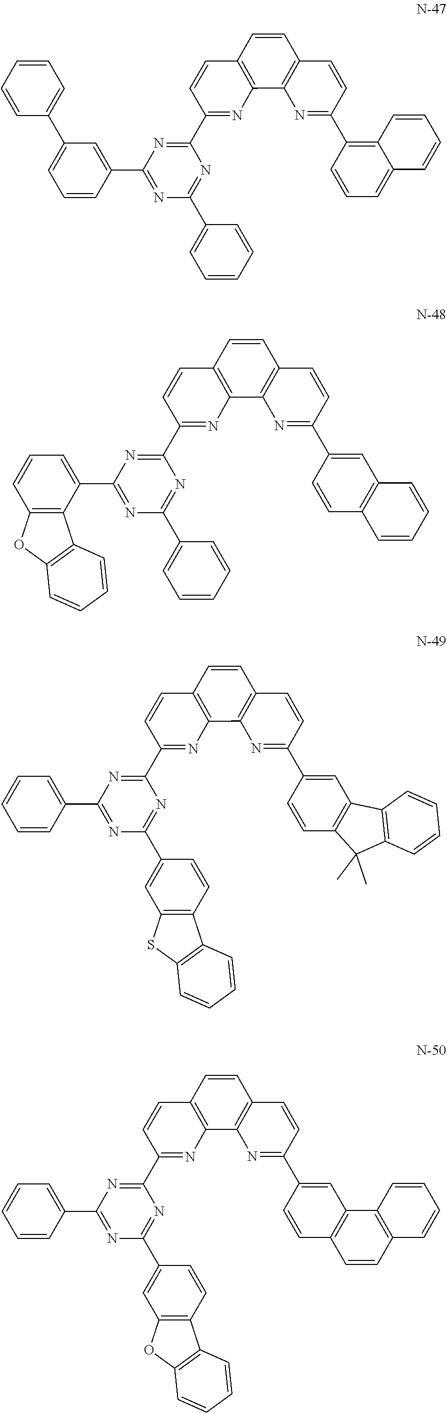 [SMILES: c1ccc(-c2cccc(-c3nc(-c4ccccc4)nc(-c4ccc5ccc6ccc(-c7cccc8ccccc78)nc6c5n4)n3)c2)cc1 ; c1ccc(-c2nc(-c3ccc4ccc5ccc(-c6ccc7ccccc7c6)nc5c4n3)nc(-c3cccc4oc5ccccc5c34)n2)cc1 ; CC1(C)c2ccccc2-c2cc(-c3ccc4ccc5ccc(-c6nc(-c7ccccc7)nc(-c7ccc8c(c7)sc7ccccc78)n6)nc5c4n3)ccc21 ; c1ccc(-c2nc(-c3ccc4c(c3)oc3ccccc34)nc(-c3ccc4ccc5ccc(-c6ccc7ccc8ccccc8c7c6)nc5c4n3)n2)cc1]

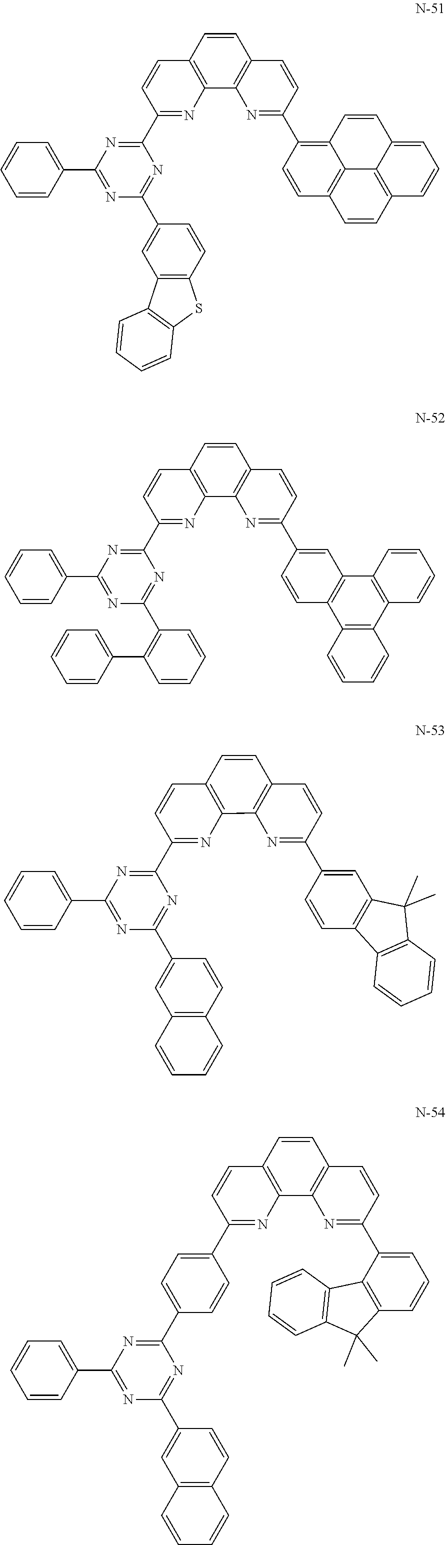 [SMILES: c1ccc(-c2nc(-c3ccc4sc5ccccc5c4c3)nc(-c3ccc4ccc5ccc(-c6ccc7ccc8cccc9ccc6c7c89)nc5c4n3)n2)cc1 ; c1ccc(-c2nc(-c3ccc4ccc5ccc(-c6ccc7c8ccccc8c8ccccc8c7c6)nc5c4n3)nc(-c3ccccc3-c3ccccc3)n2)cc1 ; CC1(C)c2ccccc2-c2ccc(-c3ccc4ccc5ccc(-c6nc(-c7ccccc7)nc(-c7ccc8ccccc8c7)n6)nc5c4n3)cc21 ; CC1(C)c2ccccc2-c2c(-c3ccc4ccc5ccc(-c6ccc(-c7nc(-c8ccccc8)nc(-c8ccc9ccccc9c8)n7)cc6)nc5c4n3)cccc21]

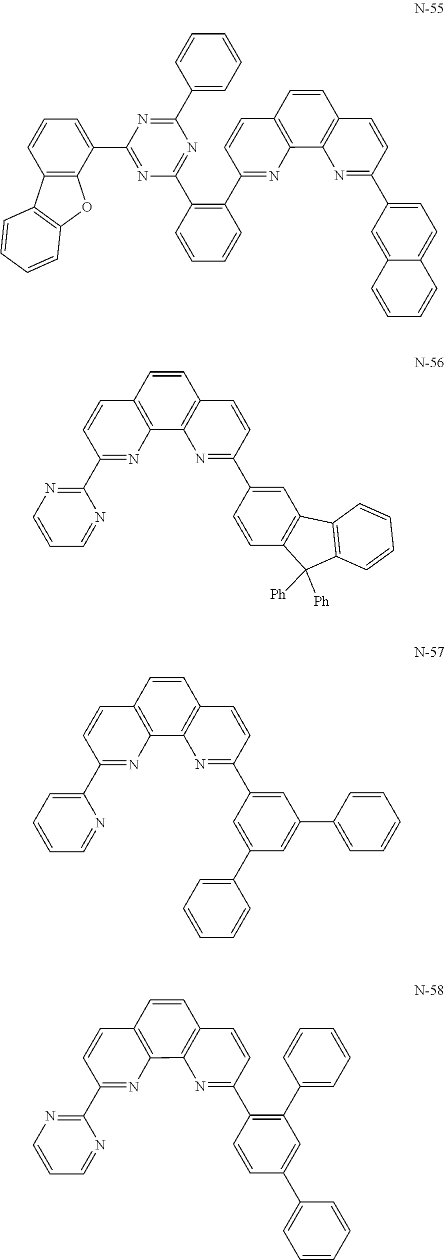 [SMILES: c1ccc(-c2nc(-c3ccccc3-c3ccc4ccc5ccc(-c6ccc7ccccc7c6)nc5c4n3)nc(-c3cccc4c3oc3ccccc34)n2)cc1 ; c1ccc(C2(c3ccccc3)c3ccccc3-c3cc(-c4ccc5ccc6ccc(-c7ncccn7)nc6c5n4)ccc32)cc1 ; c1ccc(-c2cc(-c3ccccc3)cc(-c3ccc4ccc5ccc(-c6ccccn6)nc5c4n3)c2)cc1 ; c1ccc(-c2ccc(-c3ccc4ccc5ccc(-c6ncccn6)nc5c4n3)c(-c3ccccc3)c2)cc1]

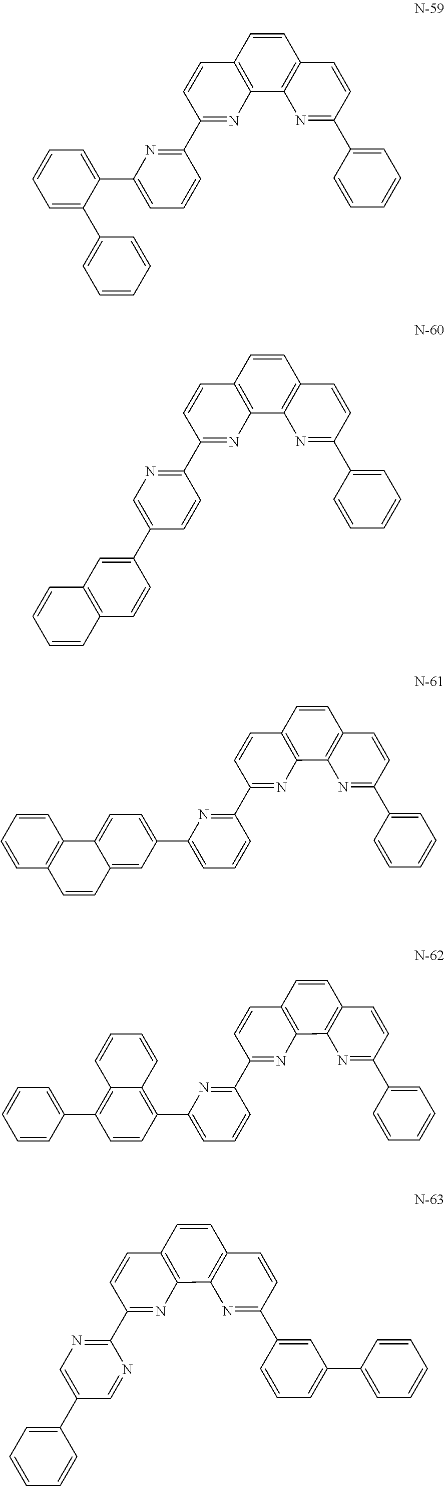 [SMILES: c1ccc(-c2ccc3ccc4ccc(-c5cccc(-c6ccccc6-c6ccccc6)n5)nc4c3n2)cc1 ; c1ccc(-c2ccc3ccc4ccc(-c5ccc(-c6ccc7ccccc7c6)cn5)nc4c3n2)cc1 ; c1ccc(-c2ccc3ccc4ccc(-c5cccc(-c6ccc7c(ccc8ccccc87)c6)n5)nc4c3n2)cc1 ; c1ccc(-c2ccc3ccc4ccc(-c5cccc(-c6ccc(-c7ccccc7)c7ccccc67)n5)nc4c3n2)cc1 ; c1ccc(-c2cnc(-c3ccc4ccc5ccc(-c6cccc(-c7ccccc7)c6)nc5c4n3)nc2)cc1]

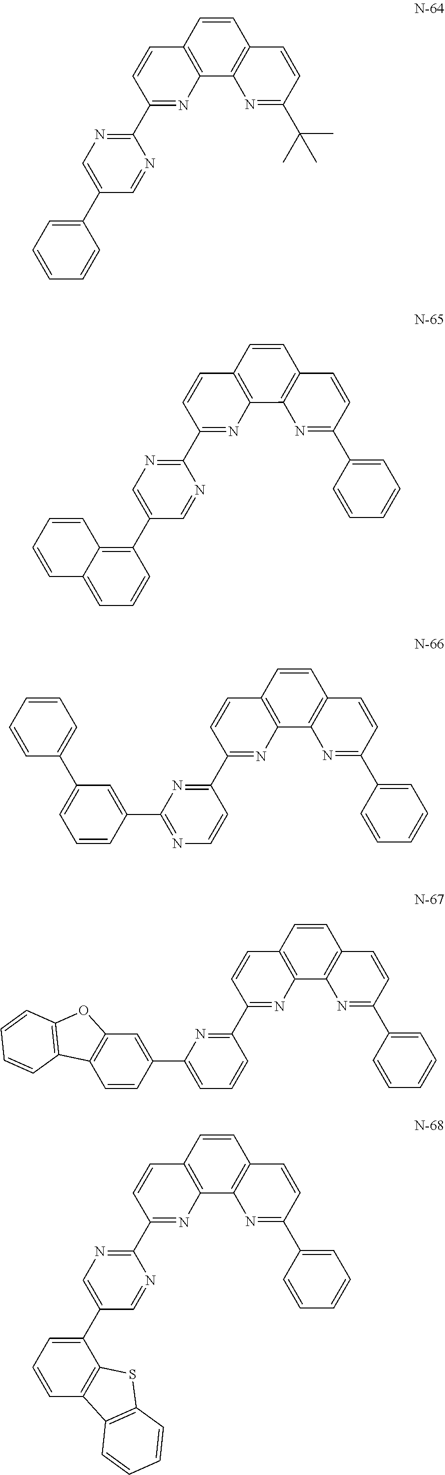

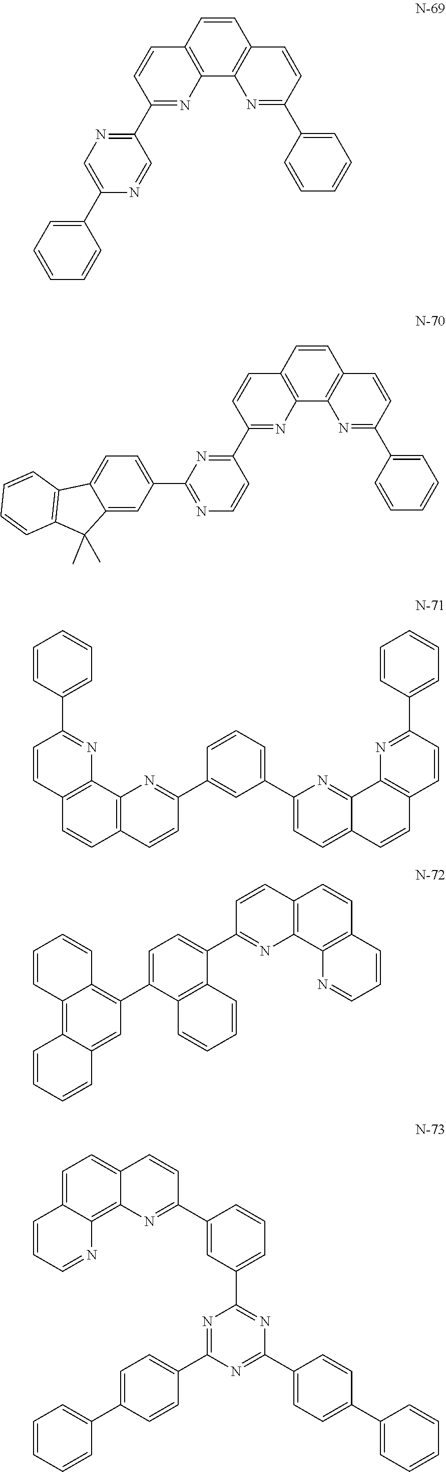

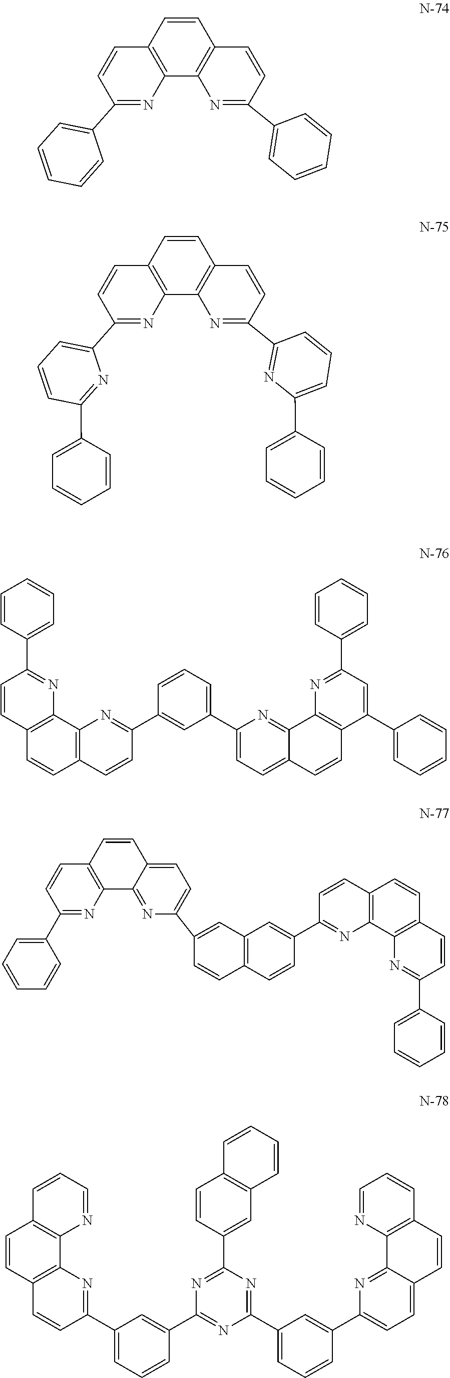 [SMILES: c1ccc(-c2ccc3ccc4ccc(-c5ccccc5)nc4c3n2)cc1 ; c1ccc(-c2cccc(-c3ccc4ccc5ccc(-c6cccc(-c7ccccc7)n6)nc5c4n3)n2)cc1 ; c1ccc(-c2ccc3ccc4ccc(-c5cccc(-c6ccc7ccc8c(-c9ccccc9)cc(-c9ccccc9)nc8c7n6)c5)nc4c3n2)cc1 ; c1ccc(-c2ccc3ccc4ccc(-c5ccc6ccc(-c7ccc8ccc9ccc(-c%10ccccc%10)nc9c8n7)cc6c5)nc4c3n2)cc1 ; c1cc(-c2ccc3ccc4cccnc4c3n2)cc(-c2nc(-c3cccc(-c4ccc5ccc6cccnc6c5n4)c3)nc(-c3ccc4ccccc4c3)n2)c1]

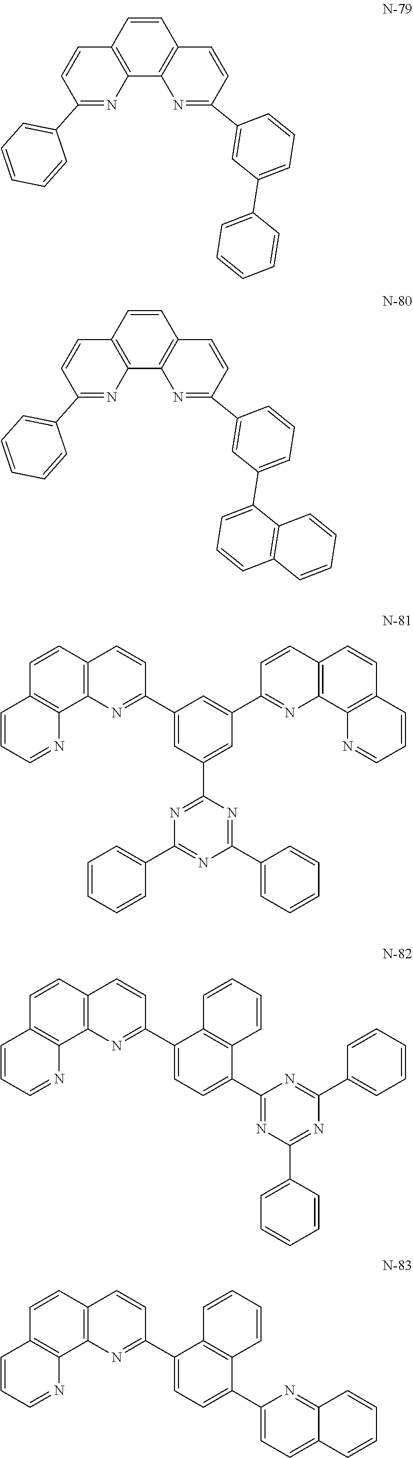 [SMILES: c1ccc(-c2cccc(-c3ccc4ccc5ccc(-c6ccccc6)nc5c4n3)c2)cc1 ; c1ccc(-c2ccc3ccc4ccc(-c5cccc(-c6cccc7ccccc67)c5)nc4c3n2)cc1 ; c1ccc(-c2nc(-c3ccccc3)nc(-c3cc(-c4ccc5ccc6cccnc6c5n4)cc(-c4ccc5ccc6cccnc6c5n4)c3)n2)cc1 ; c1ccc(-c2nc(-c3ccccc3)nc(-c3ccc(-c4ccc5ccc6cccnc6c5n4)c4ccccc34)n2)cc1 ; c1ccc2nc(-c3ccc(-c4ccc5ccc6cccnc6c5n4)c4ccccc34)ccc2c1]

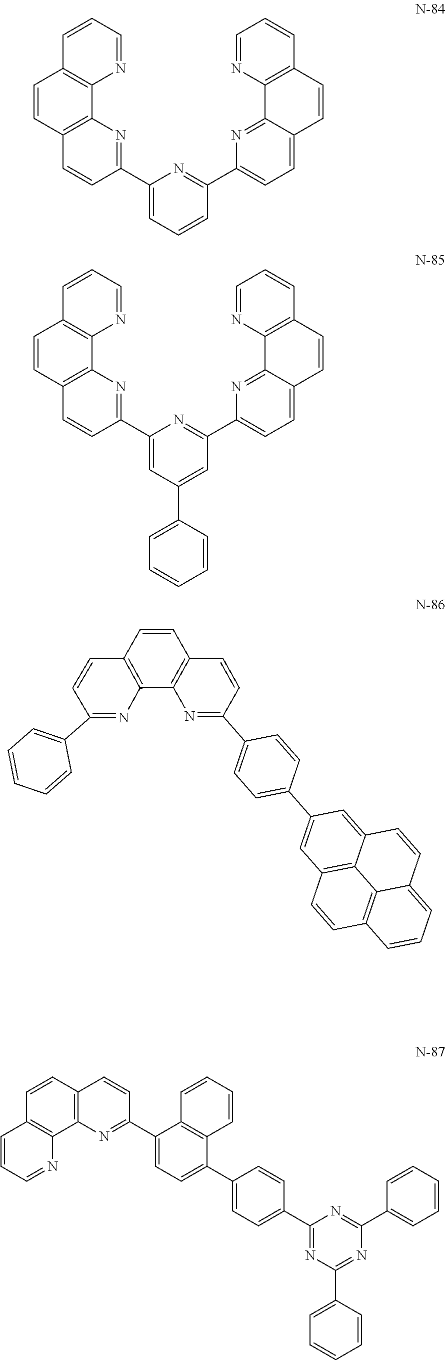

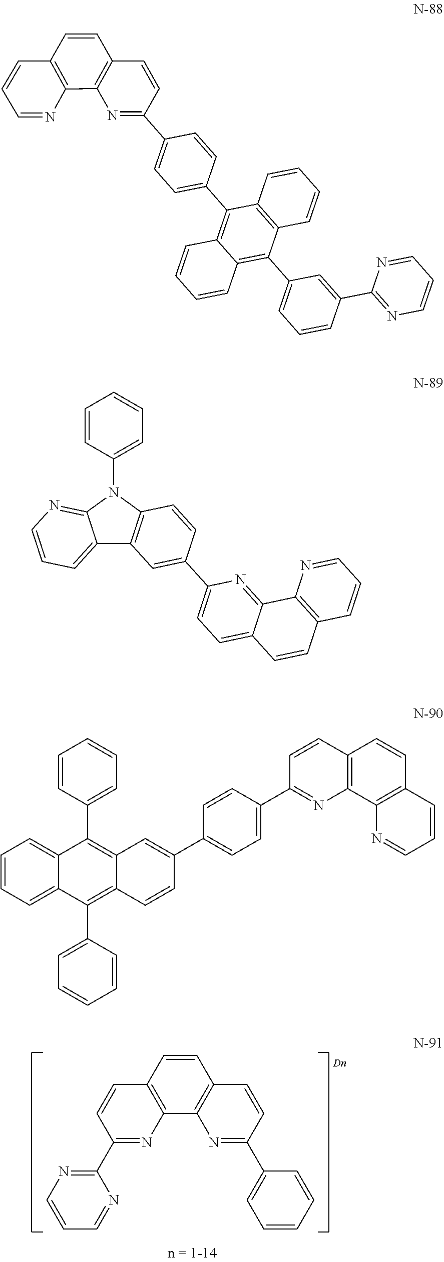

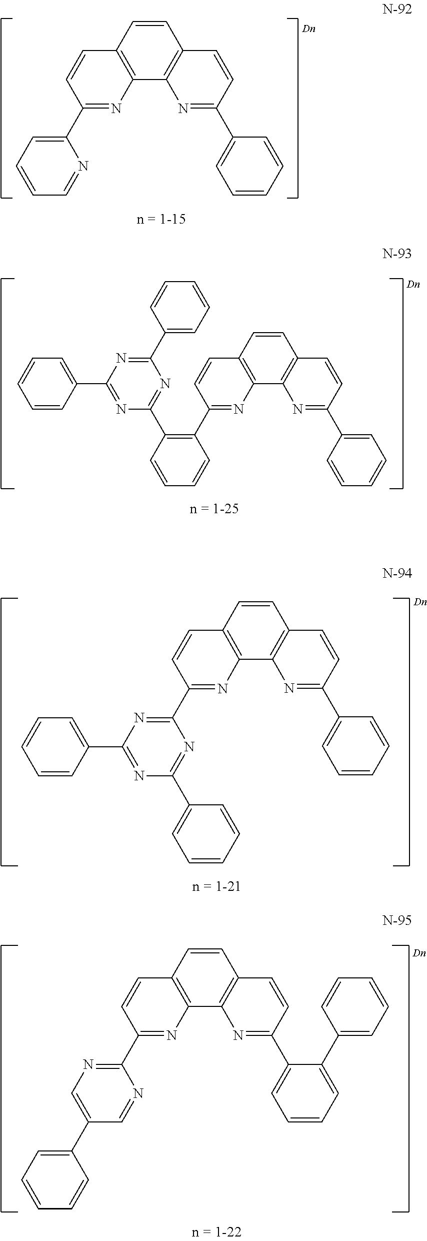

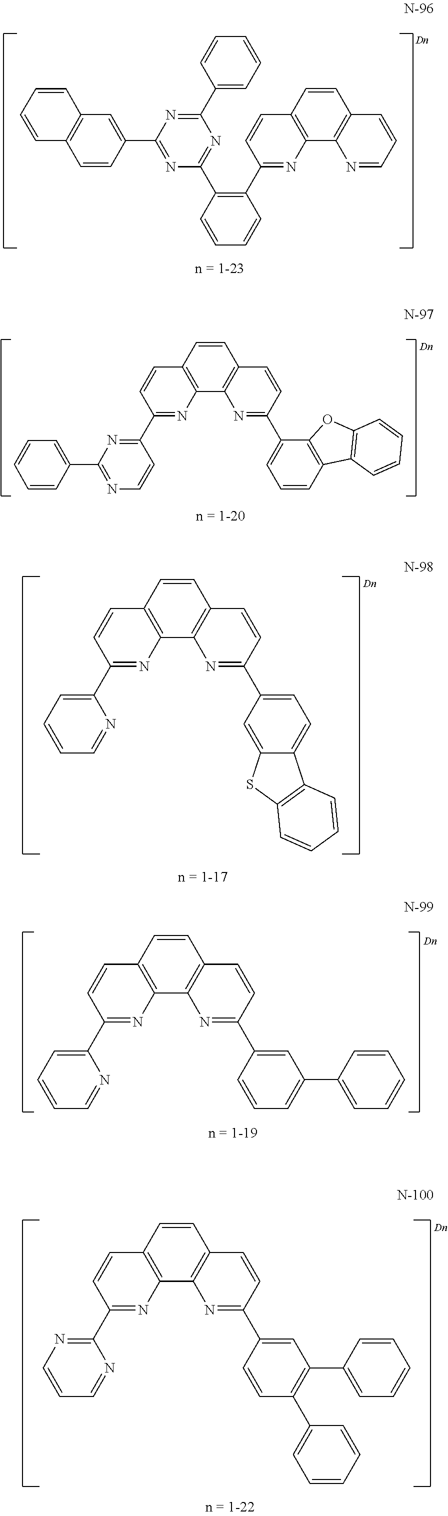

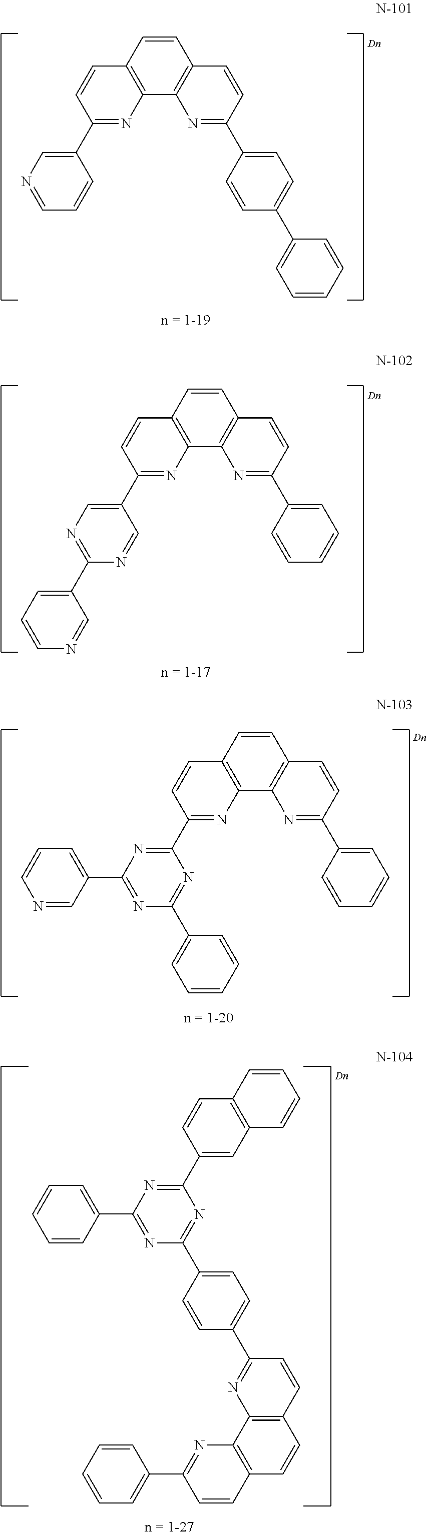

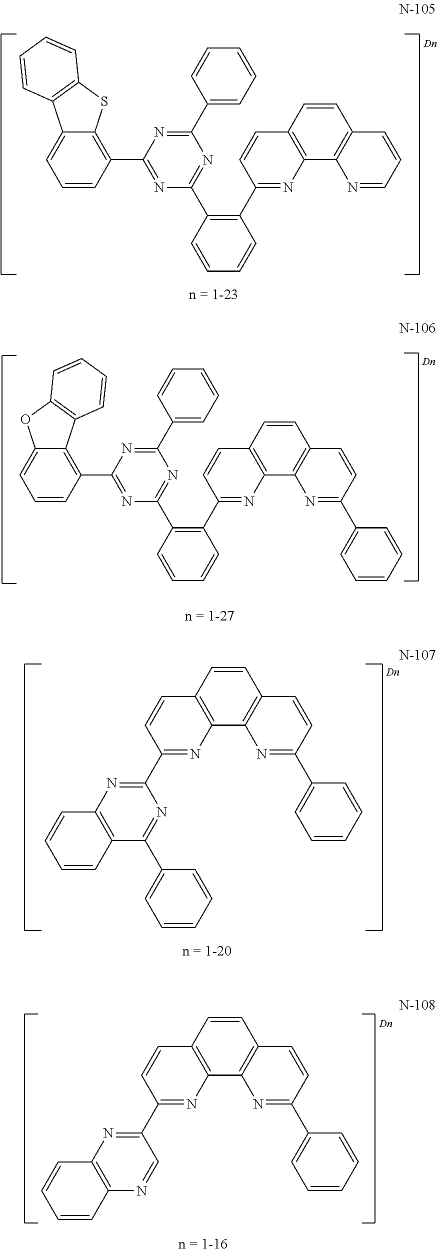

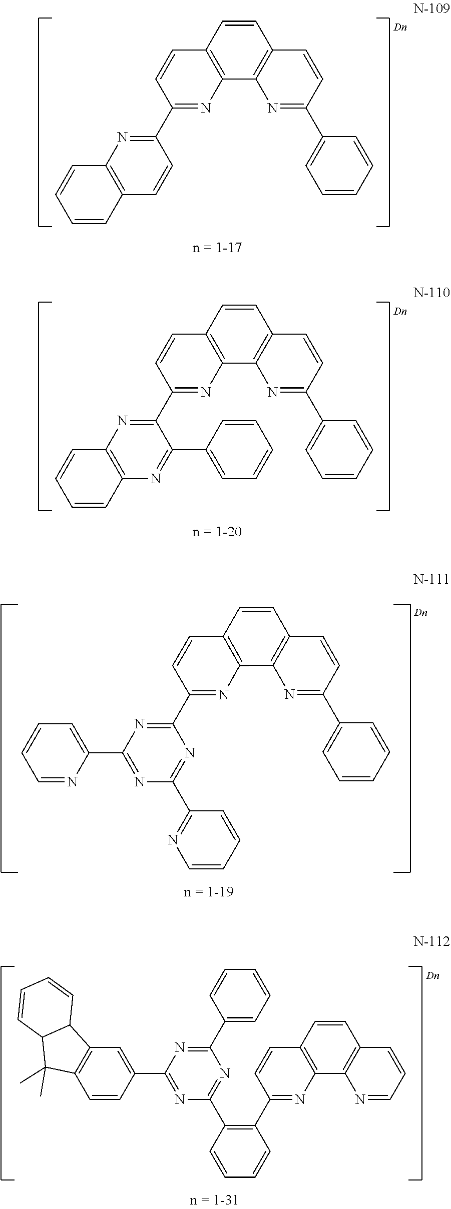

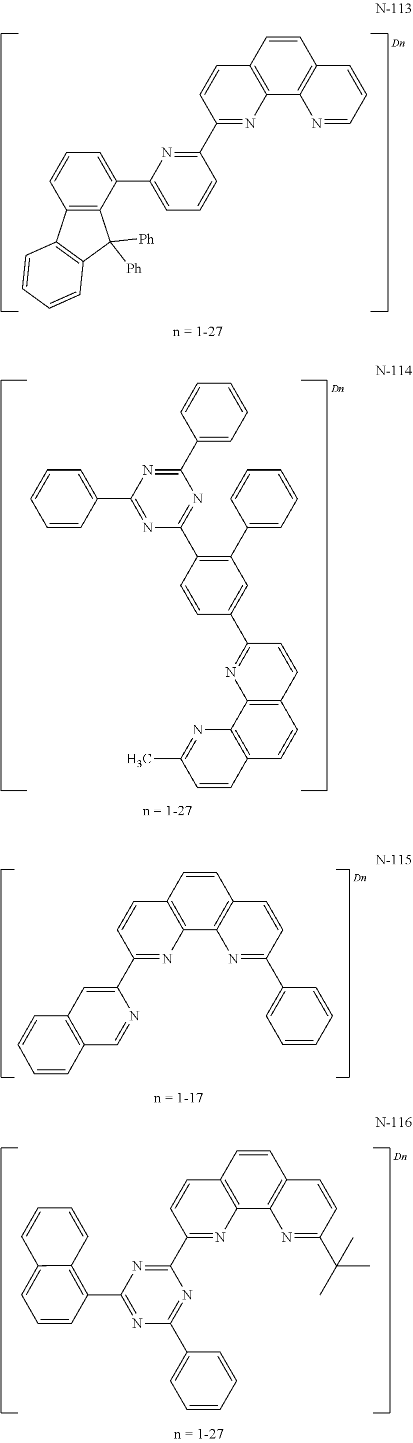

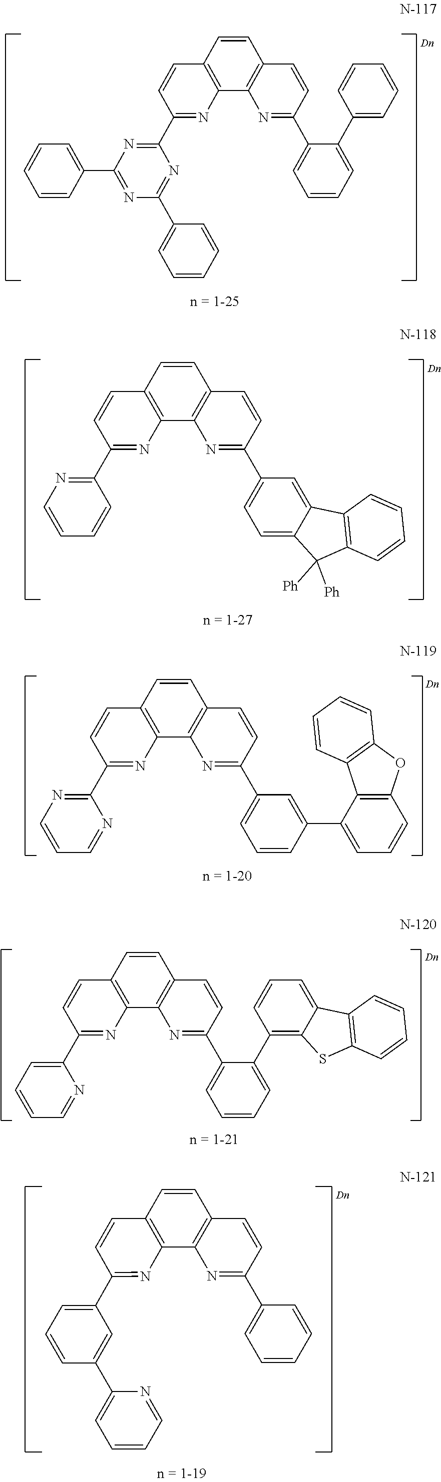

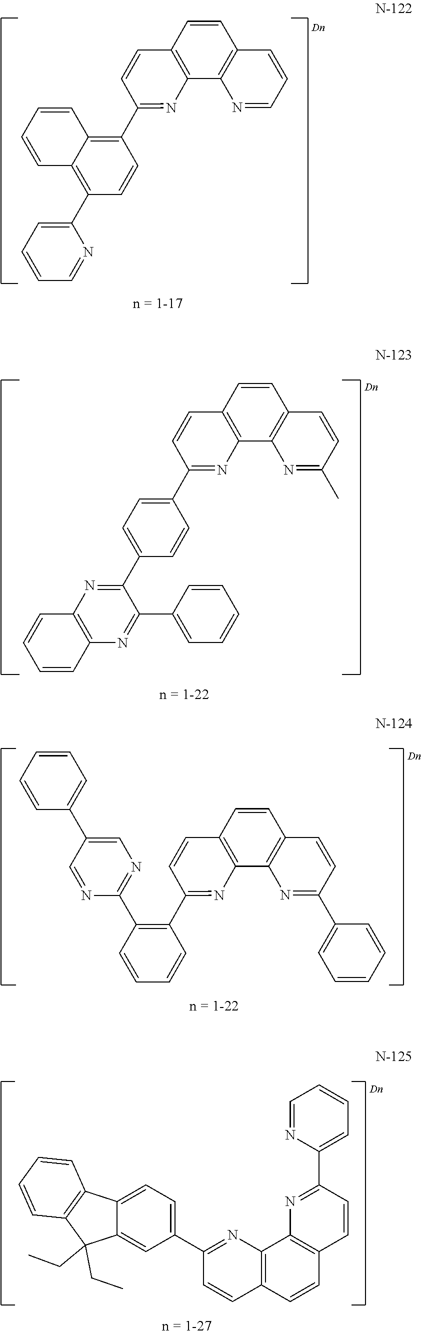

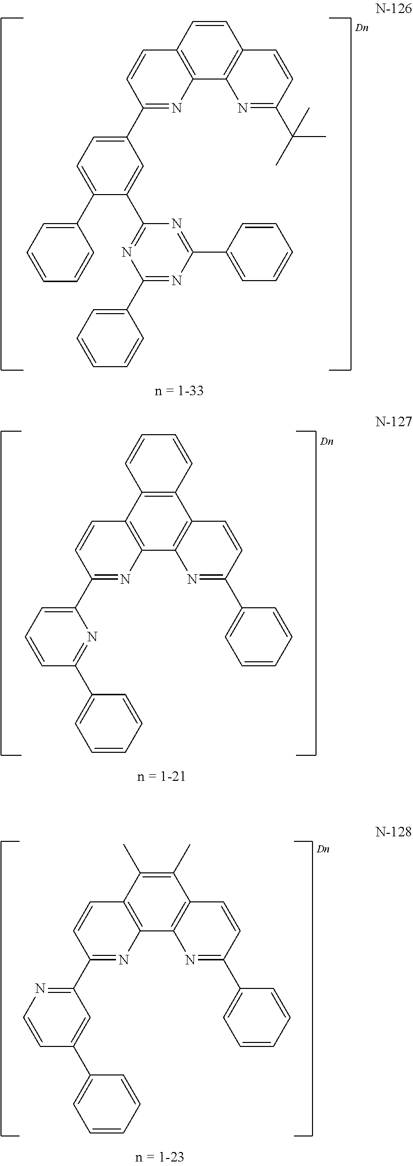

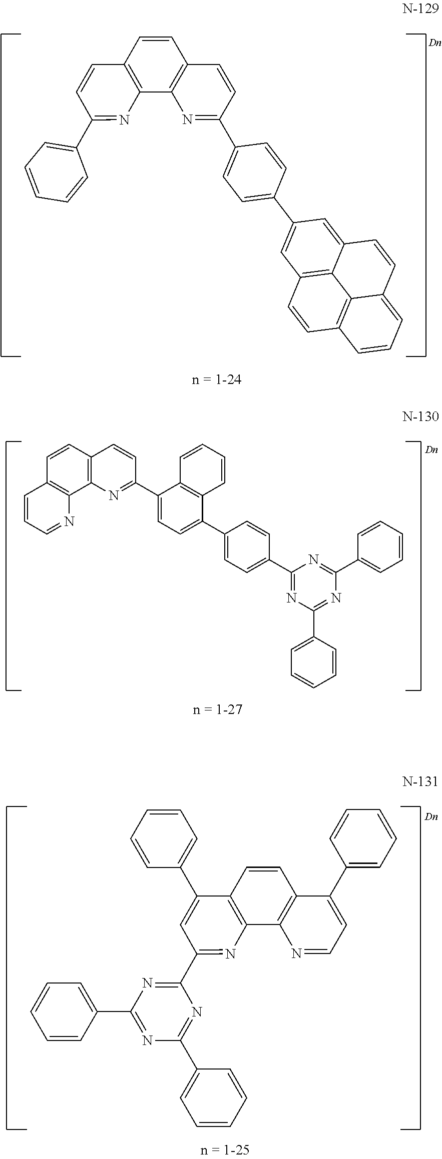

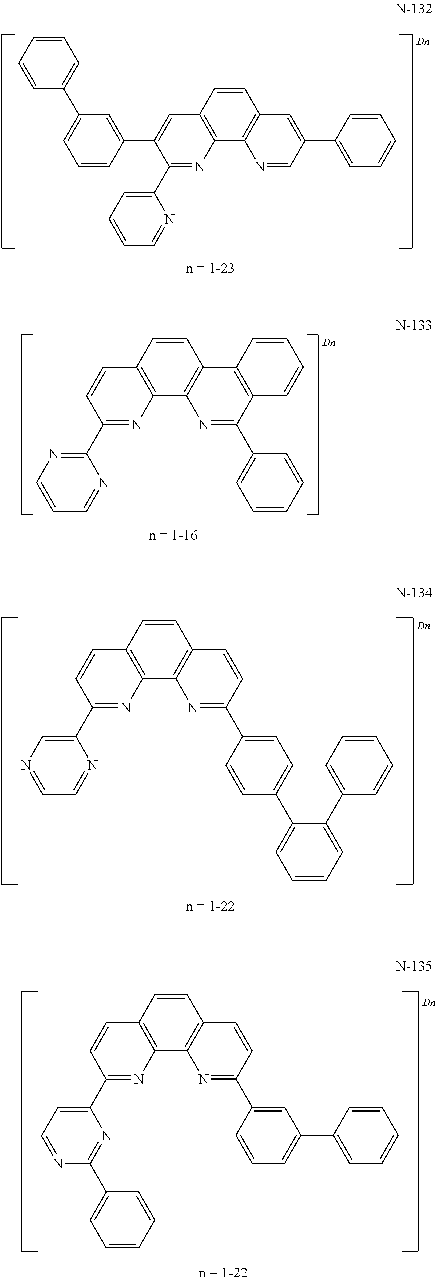

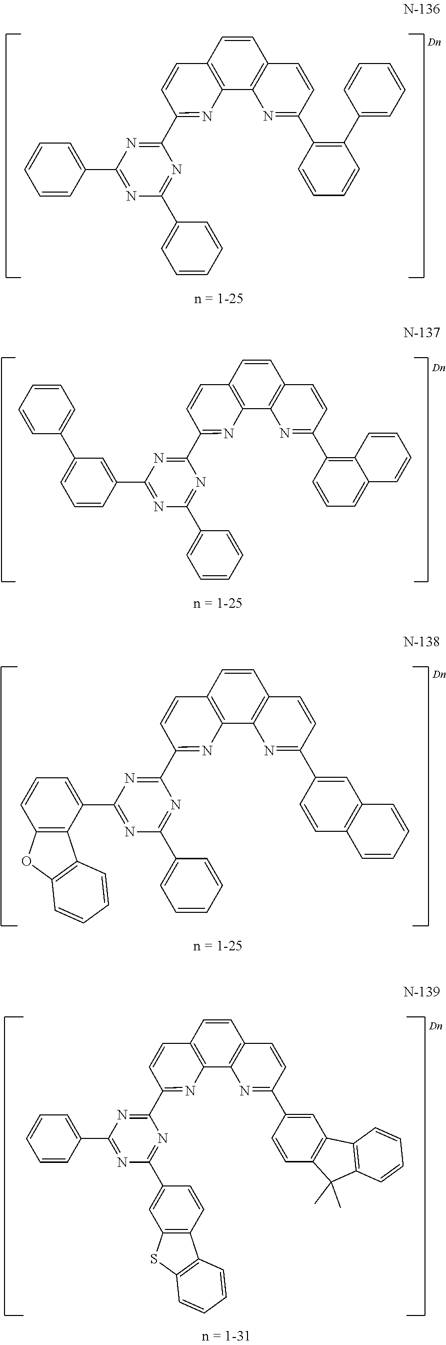

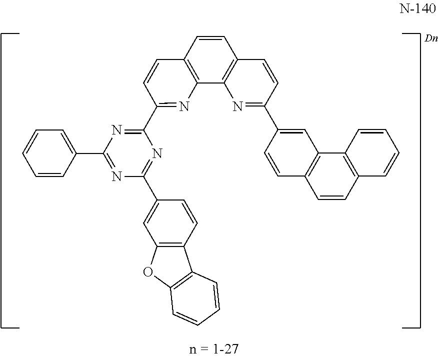

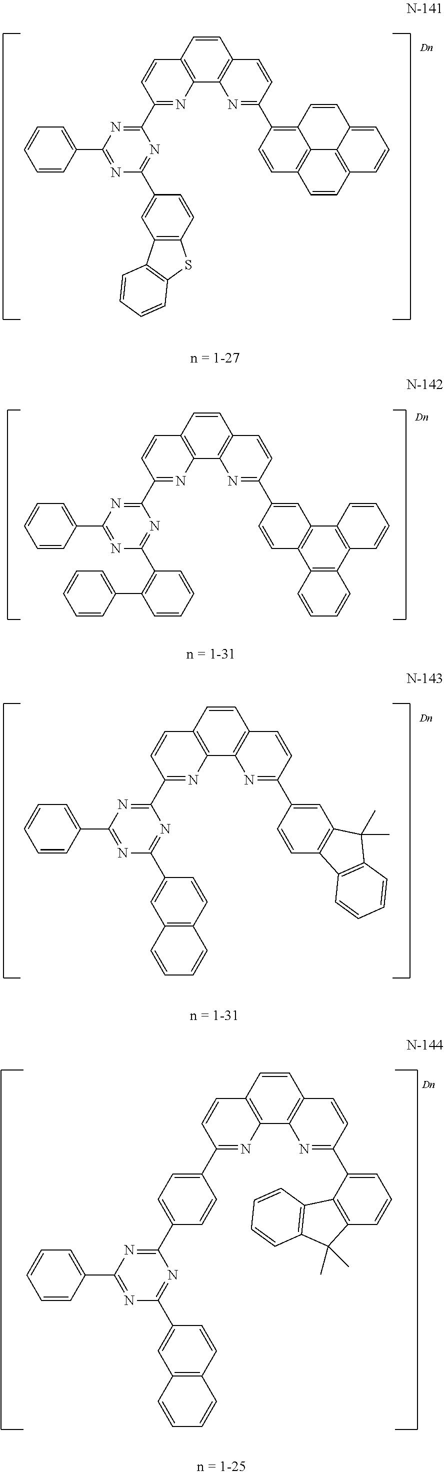

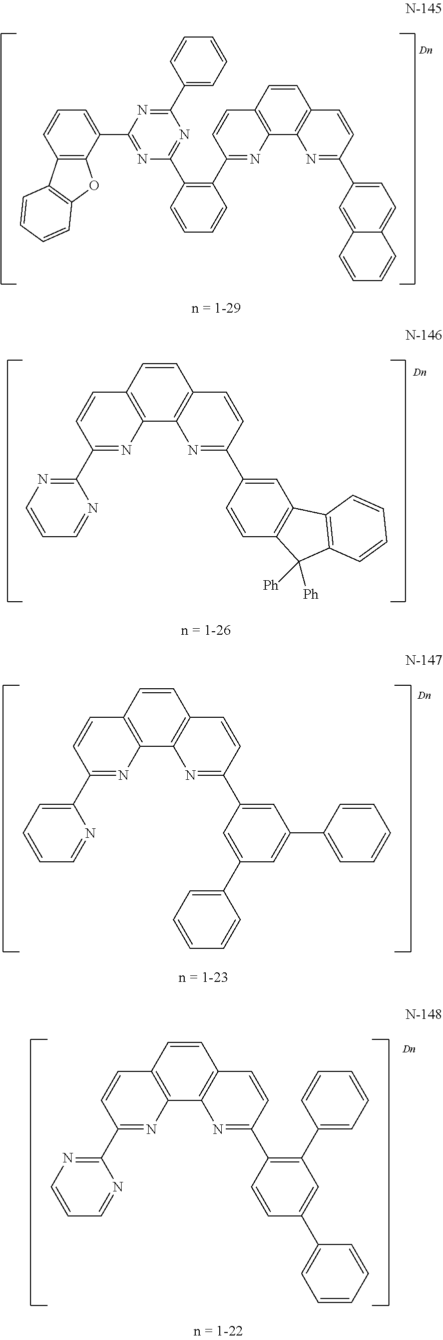

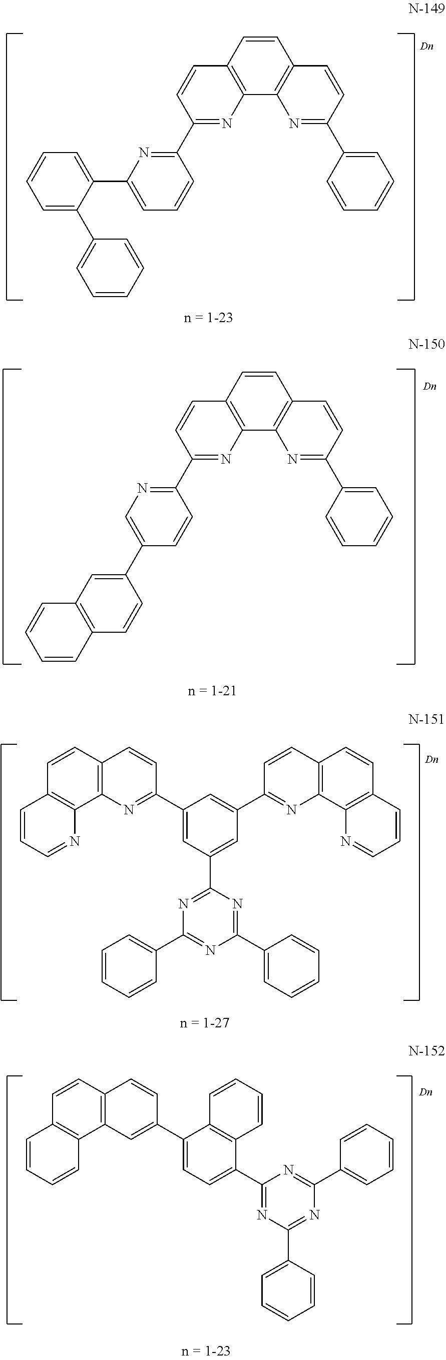

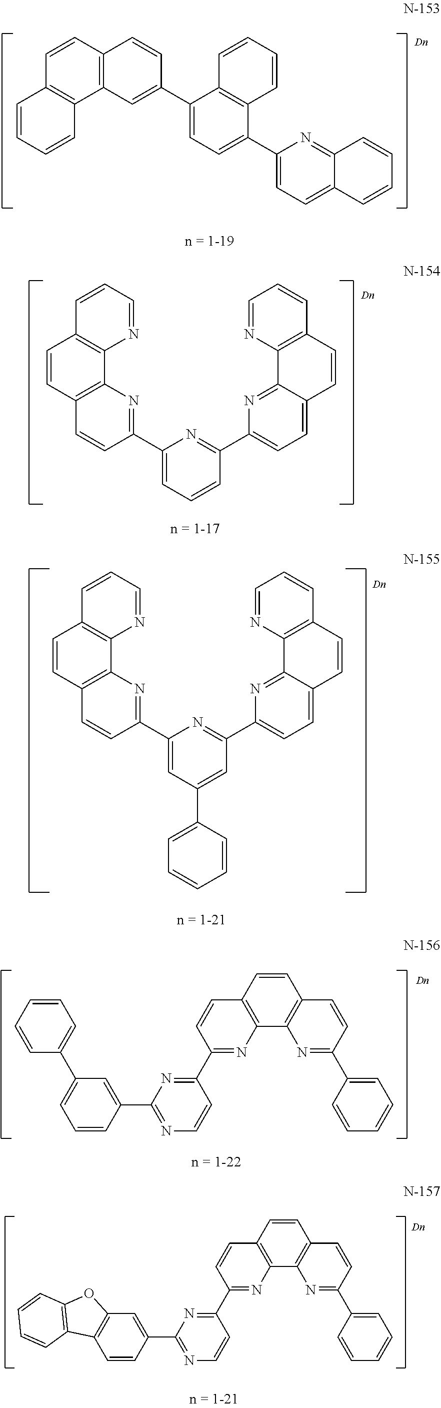

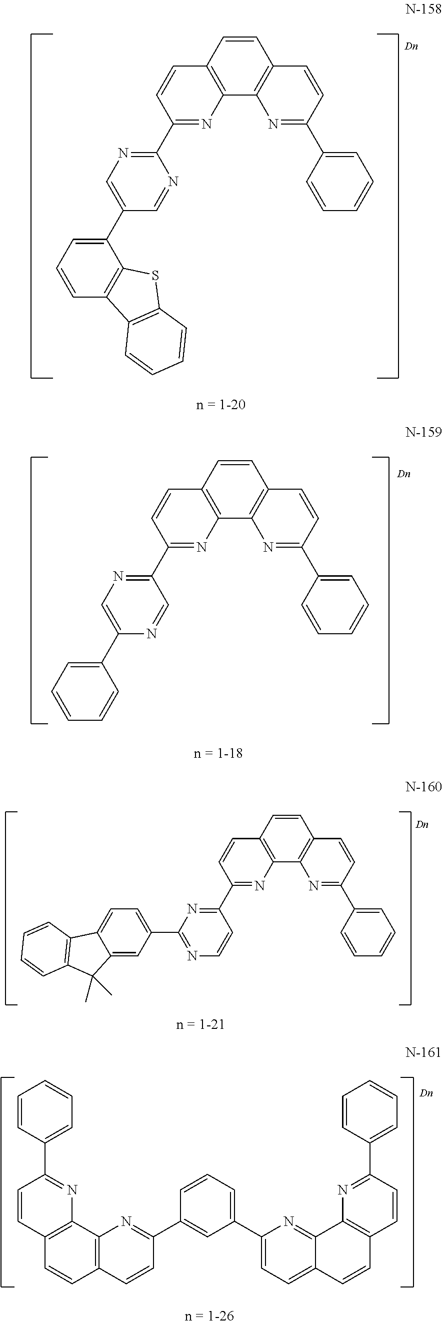

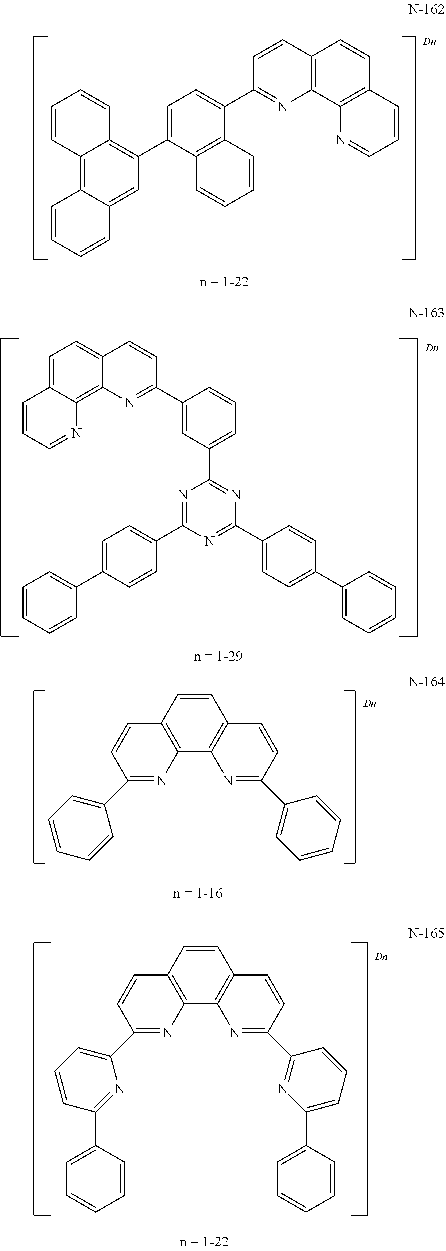

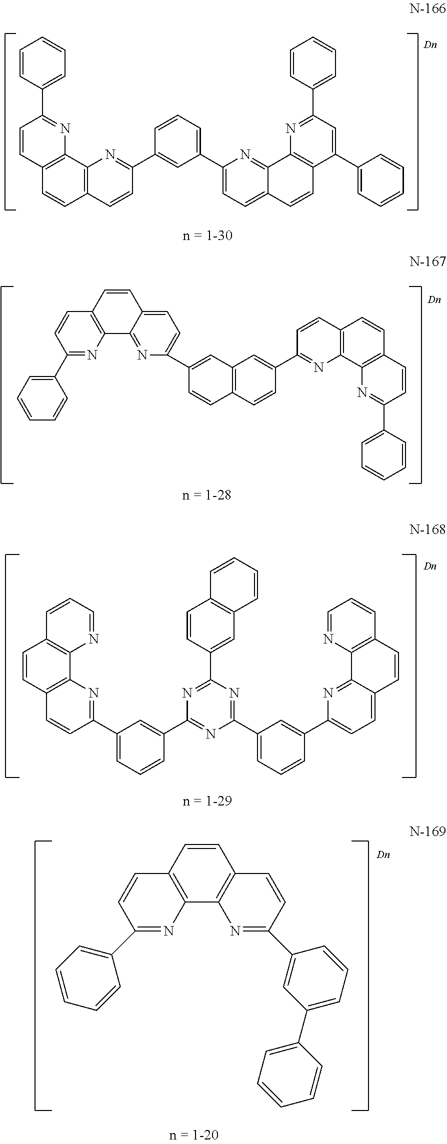

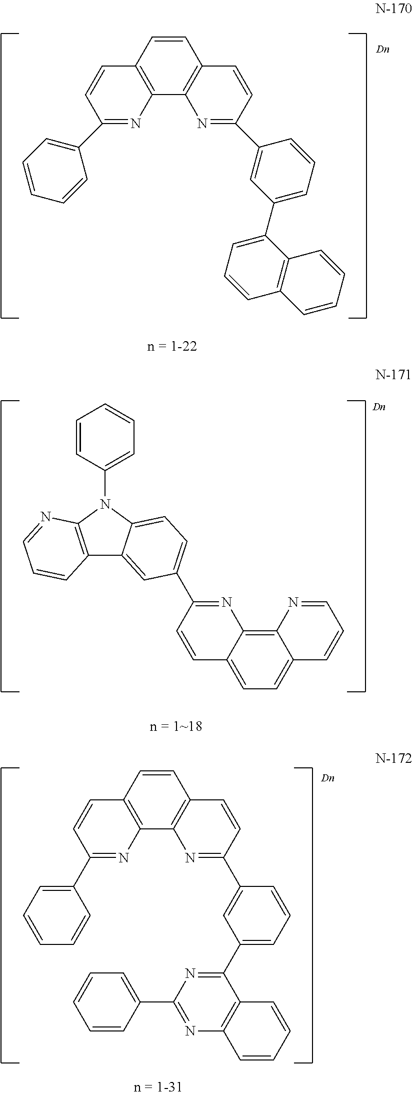

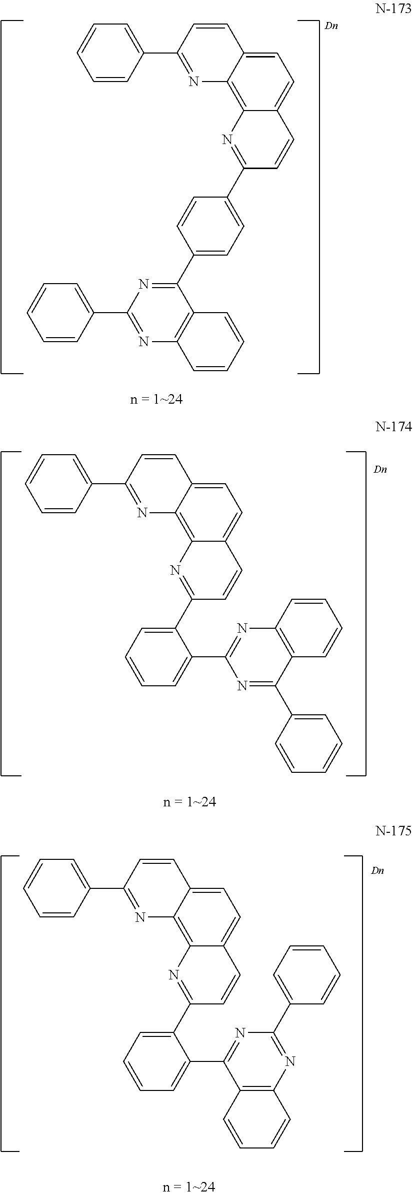

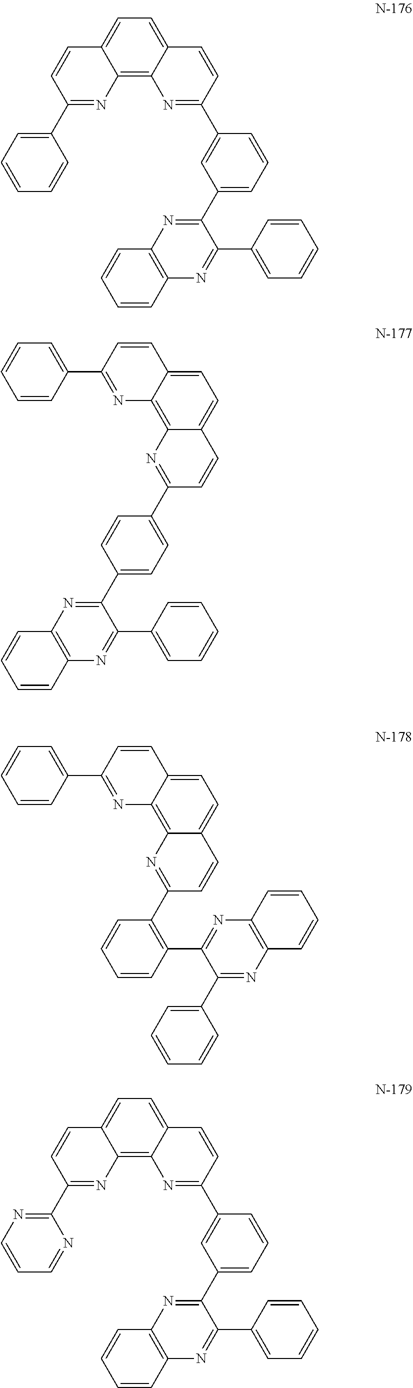

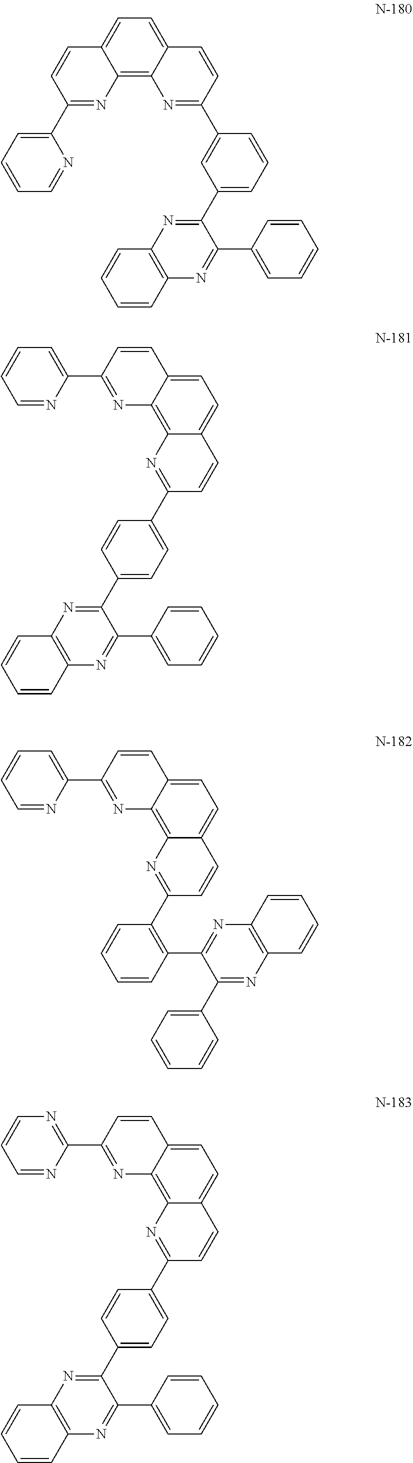

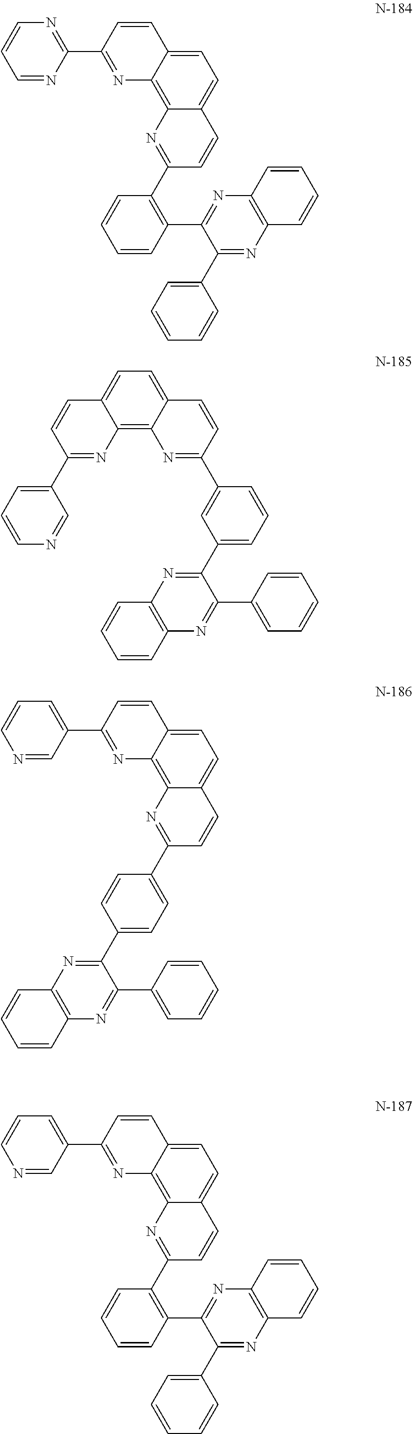

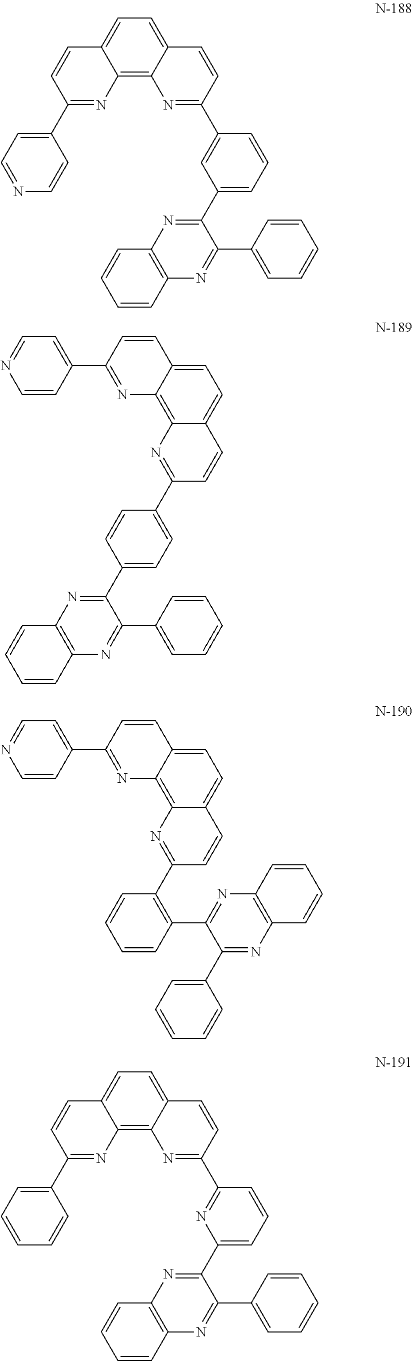

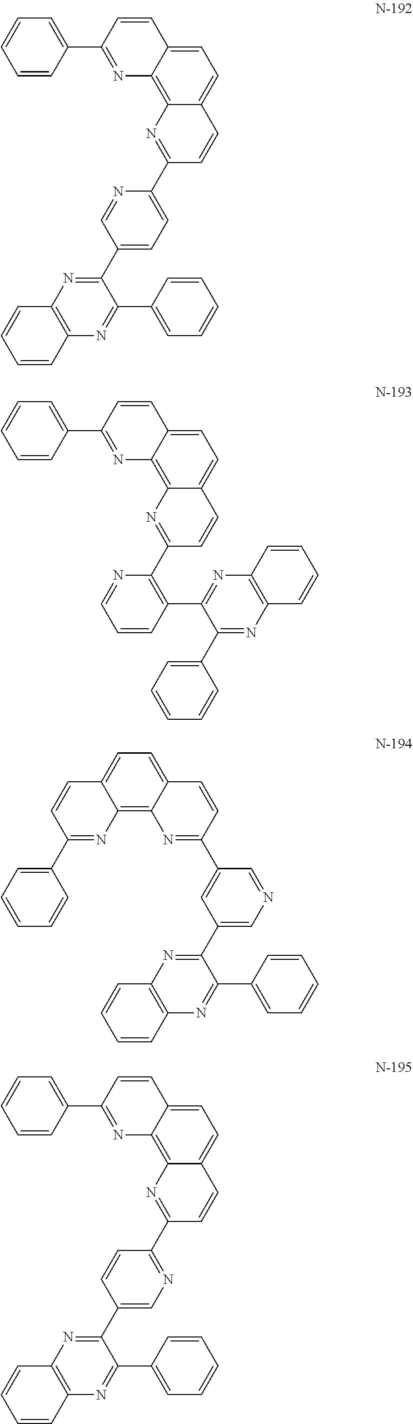

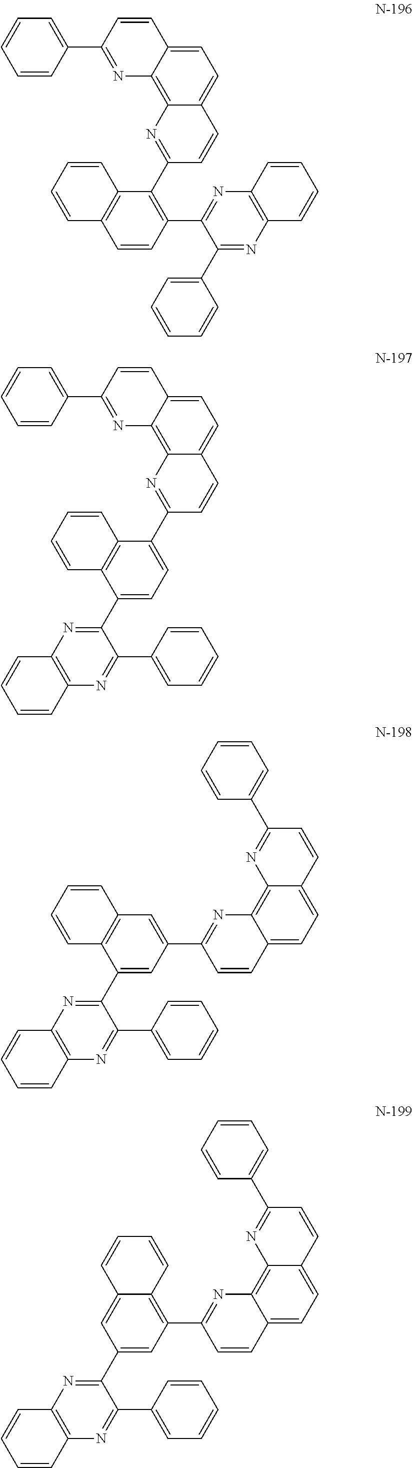

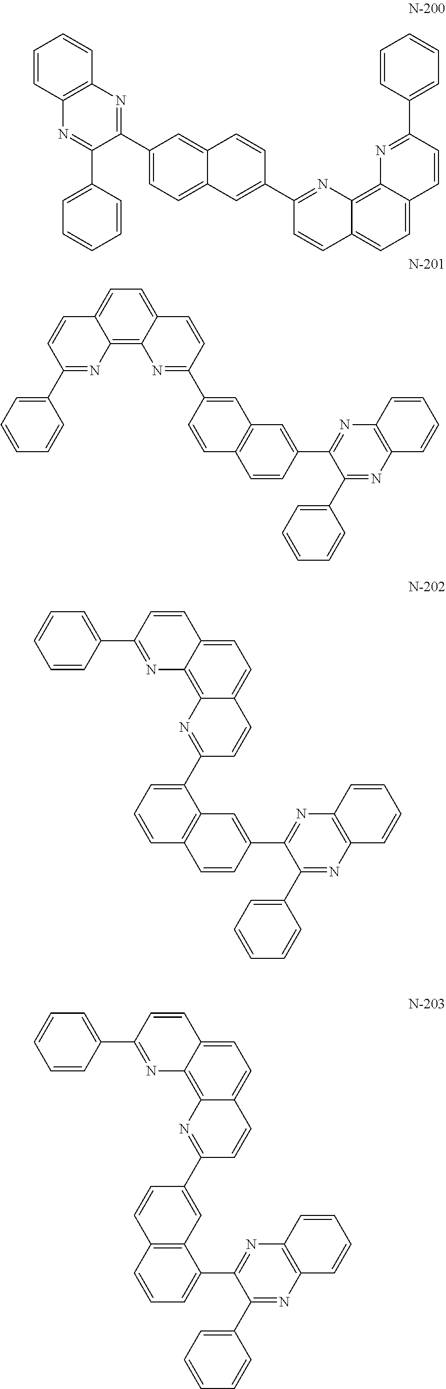

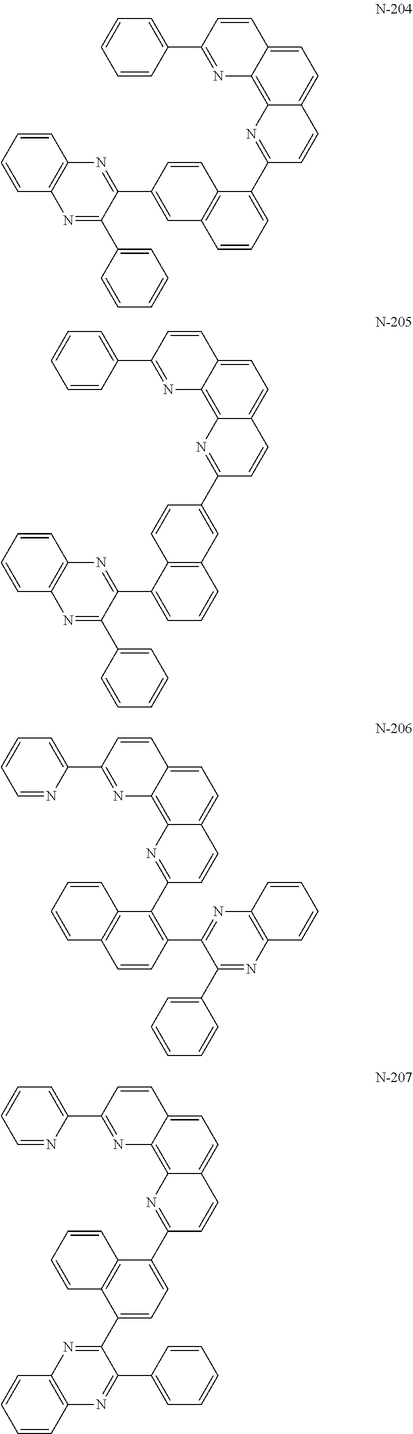

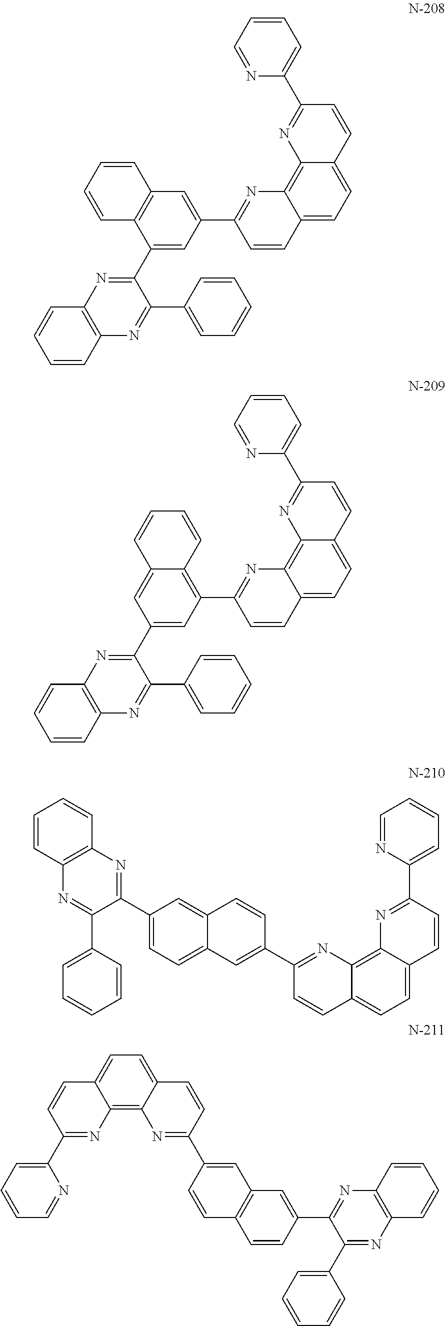

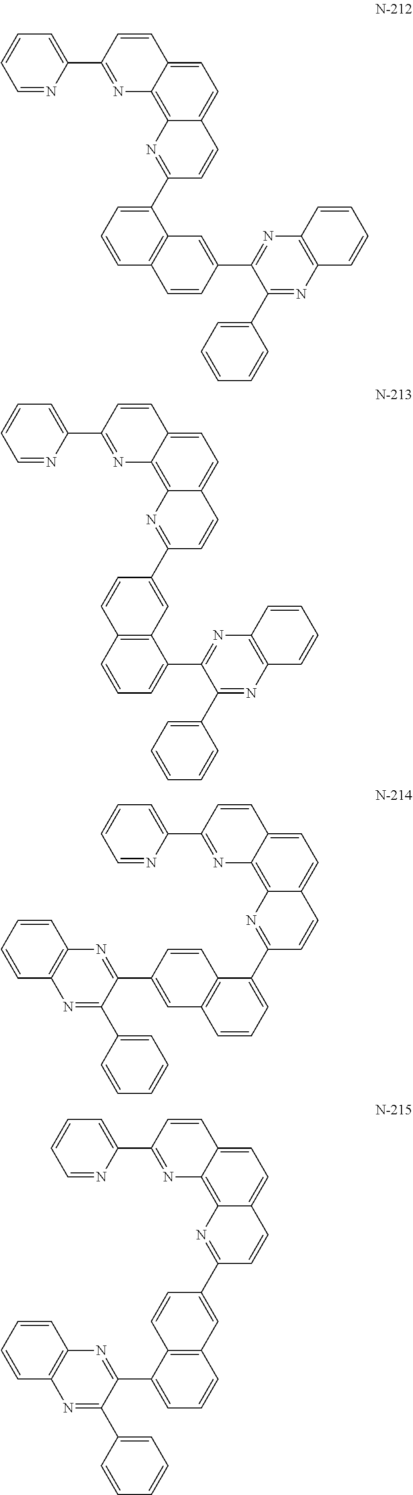

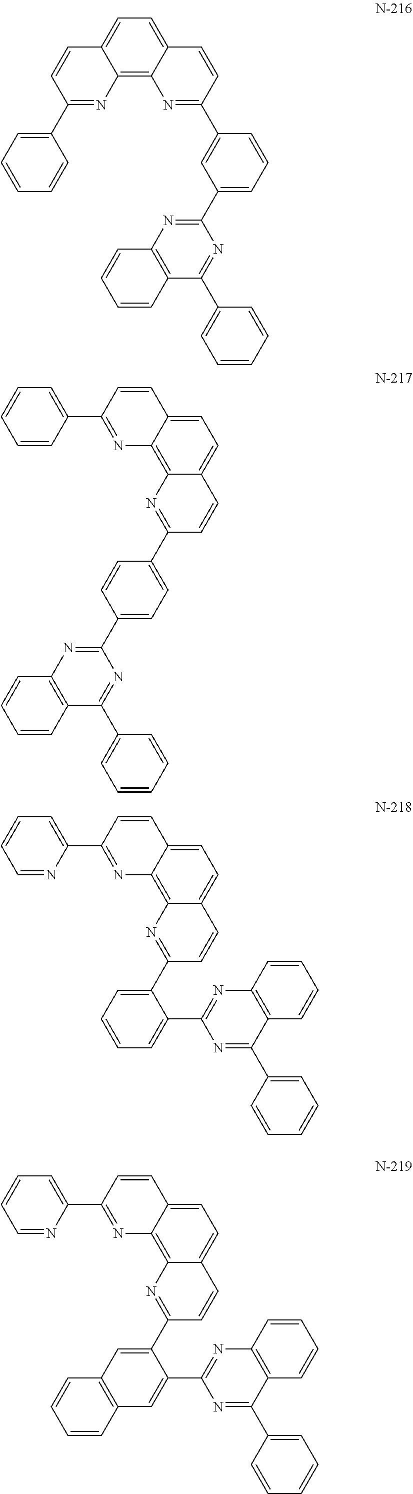

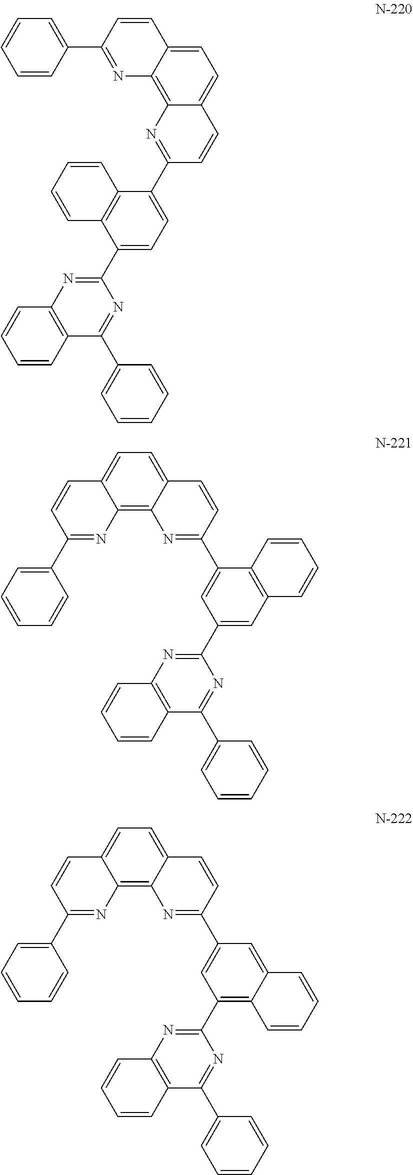

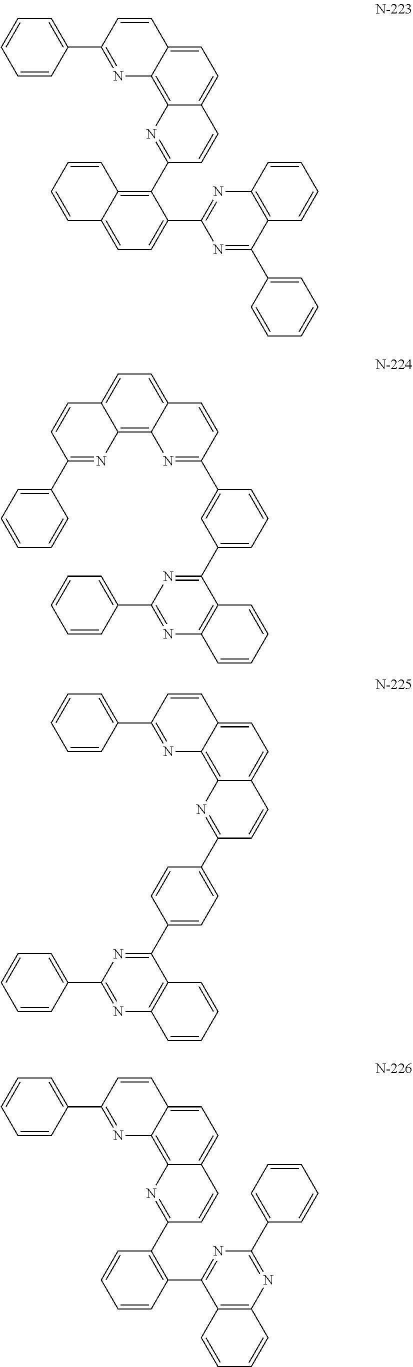

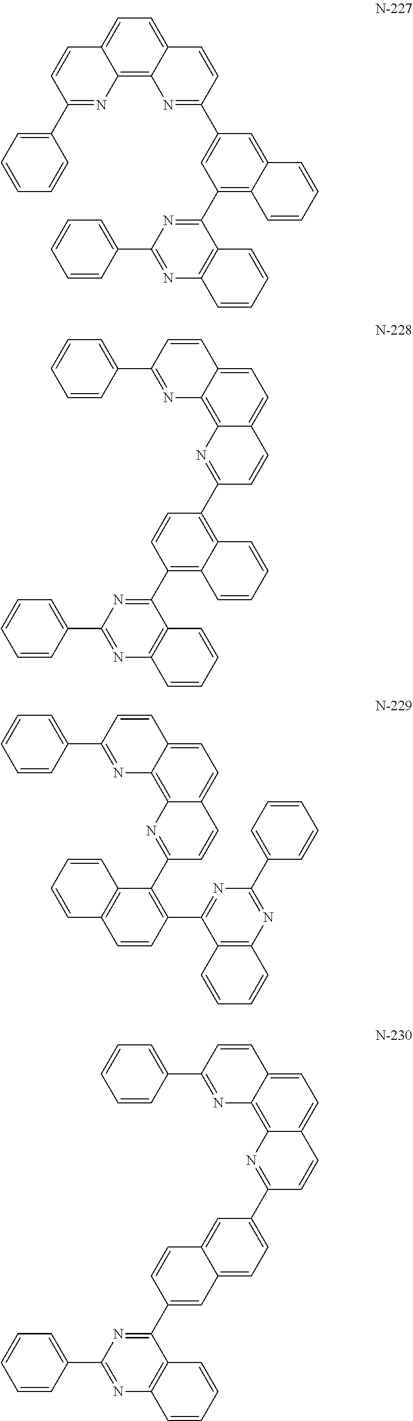

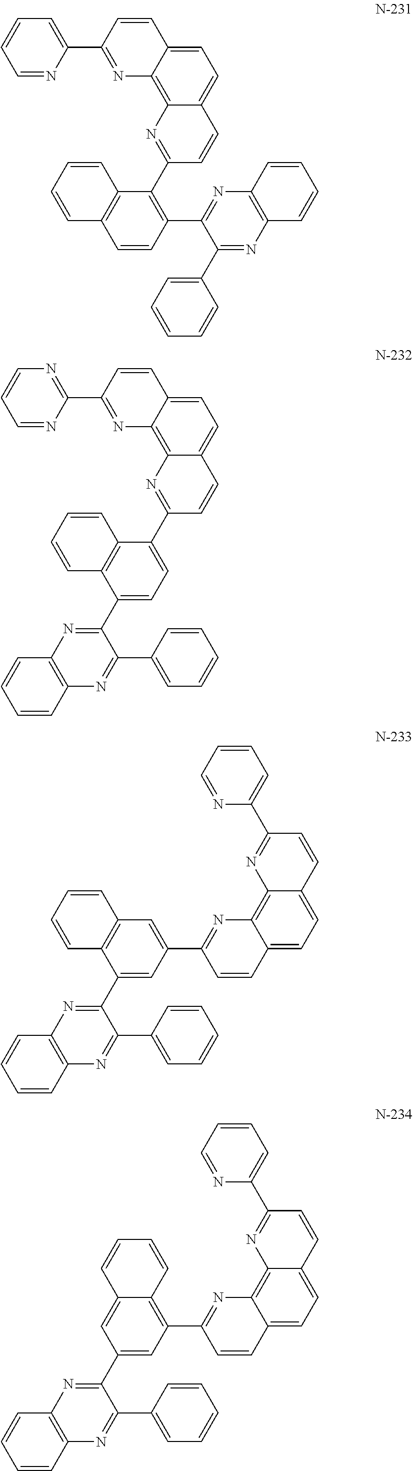

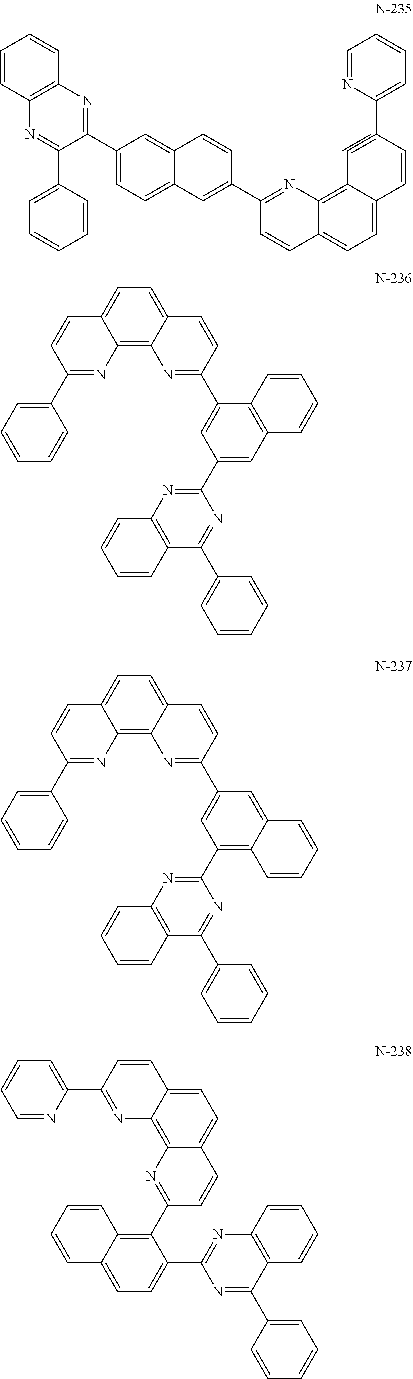

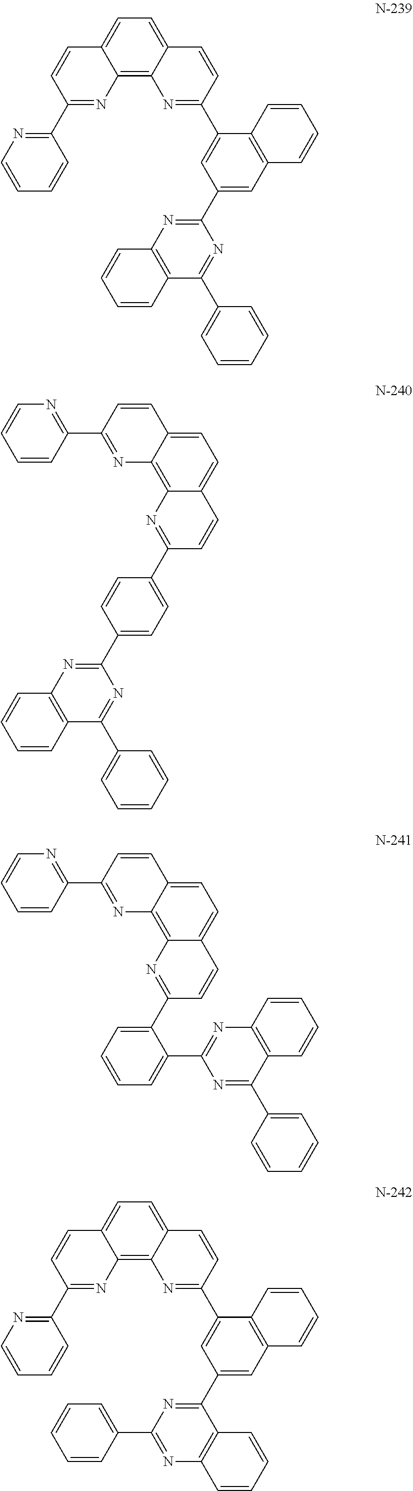

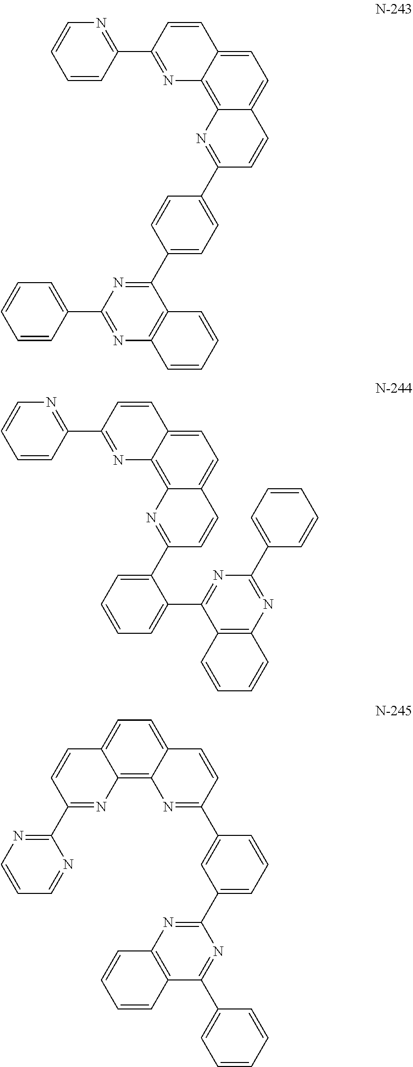

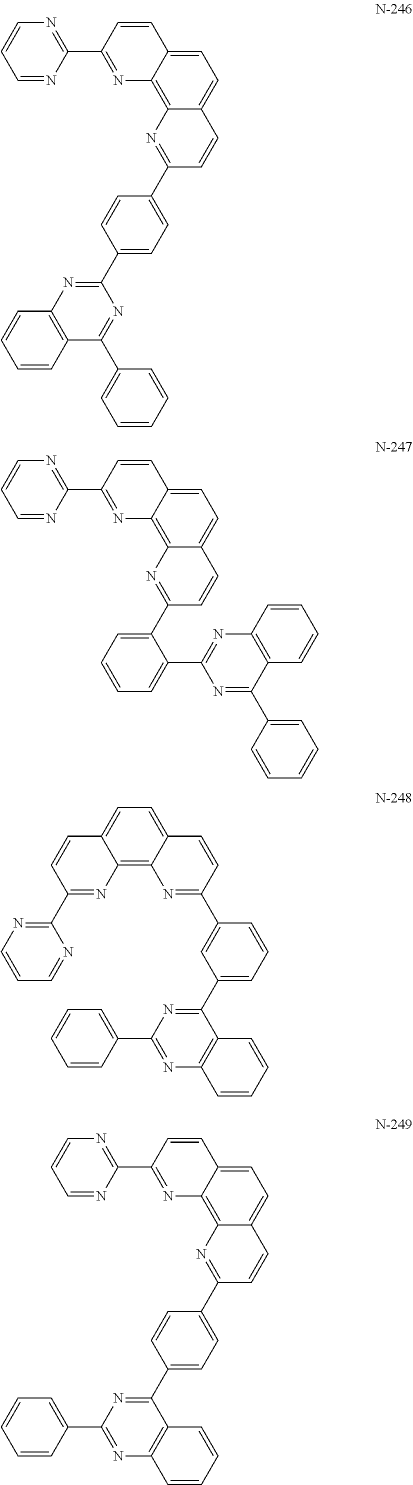

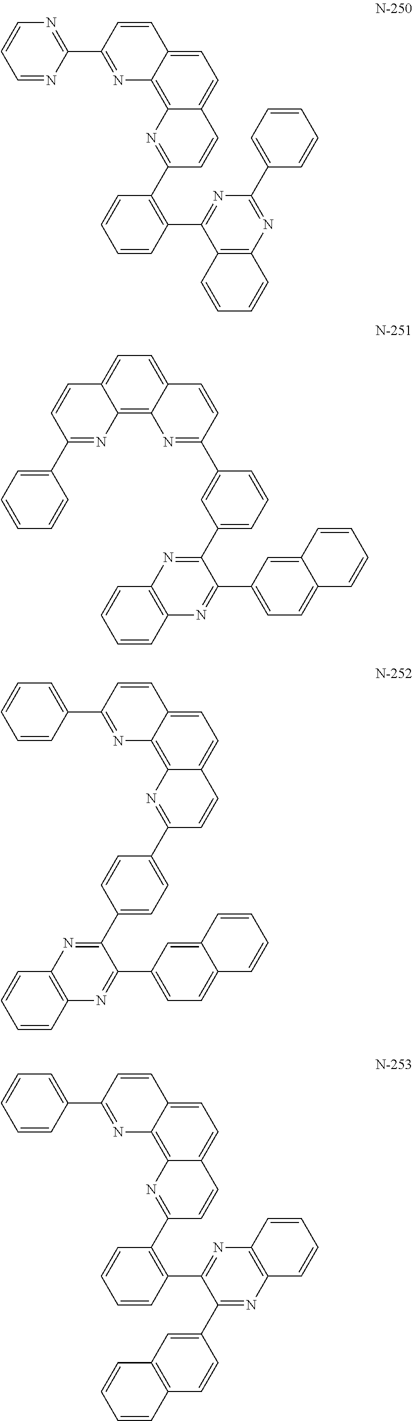

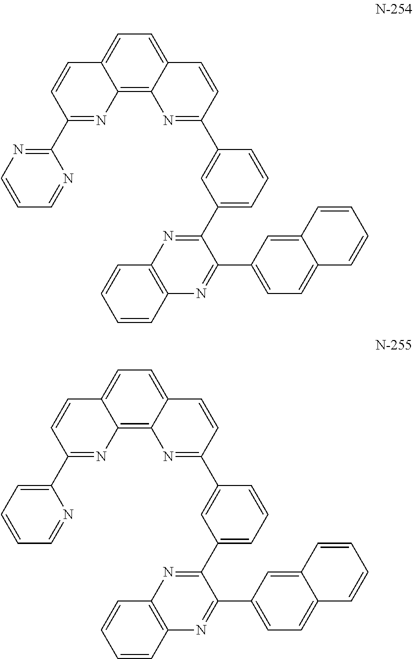 [SMILES: c1cnc(-c2ccc3ccc4ccc(-c5cccc(-c6nc7ccccc7nc6-c6ccc7ccccc7c6)c5)nc4c3n2)nc1 ; c1ccc(-c2ccc3ccc4ccc(-c5cccc(-c6nc7ccccc7nc6-c6ccc7ccccc7c6)c5)nc4c3n2)nc1]

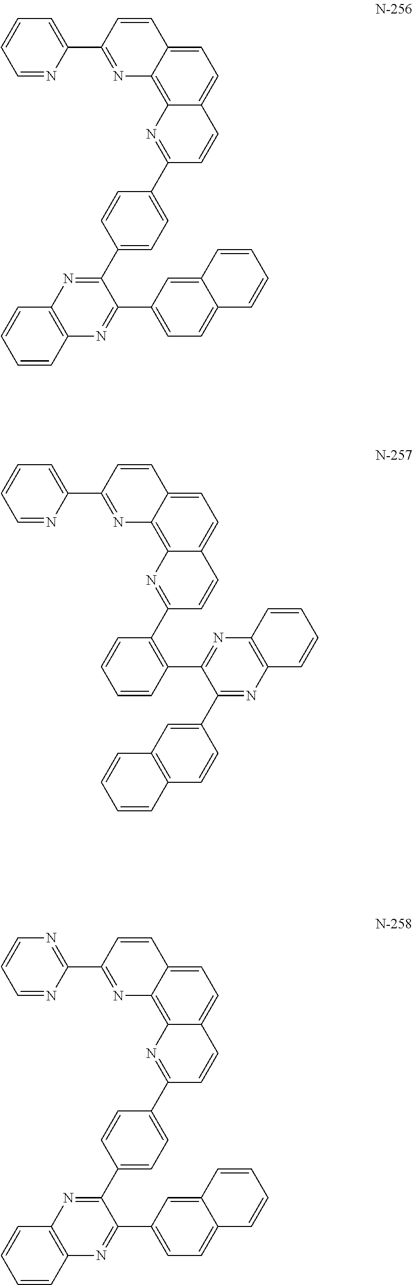 [SMILES: c1ccc(-c2ccc3ccc4ccc(-c5ccc(-c6nc7ccccc7nc6-c6ccc7ccccc7c6)cc5)nc4c3n2)nc1 ; c1ccc(-c2ccc3ccc4ccc(-c5ccccc5-c5nc6ccccc6nc5-c5ccc6ccccc6c5)nc4c3n2)nc1 ; c1cnc(-c2ccc3ccc4ccc(-c5ccc(-c6nc7ccccc7nc6-c6ccc7ccccc7c6)cc5)nc4c3n2)nc1]

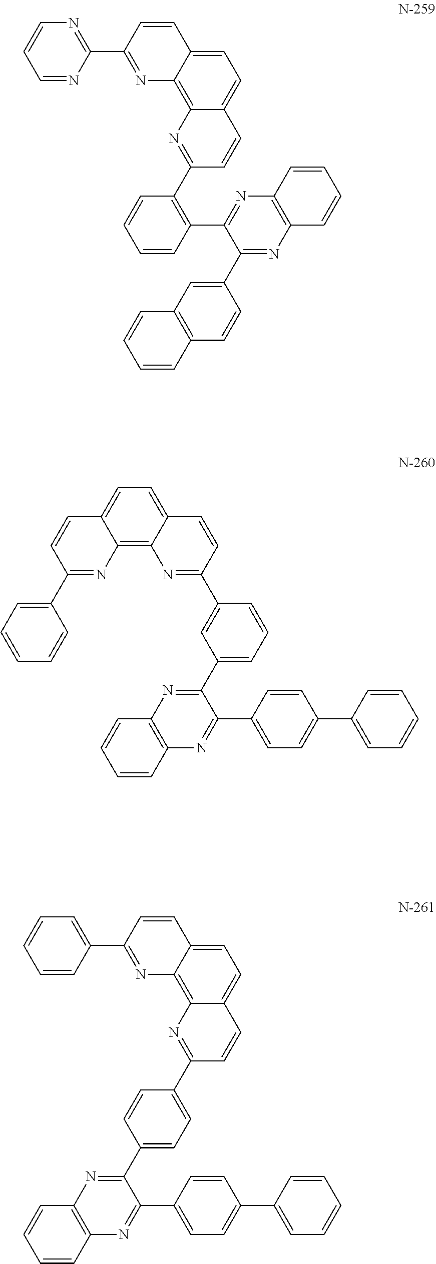 [SMILES: c1cnc(-c2ccc3ccc4ccc(-c5ccccc5-c5nc6ccccc6nc5-c5ccc6ccccc6c5)nc4c3n2)nc1 ; c1ccc(-c2ccc(-c3nc4ccccc4nc3-c3cccc(-c4ccc5ccc6ccc(-c7ccccc7)nc6c5n4)c3)cc2)cc1 ; c1ccc(-c2ccc(-c3nc4ccccc4nc3-c3ccc(-c4ccc5ccc6ccc(-c7ccccc7)nc6c5n4)cc3)cc2)cc1]

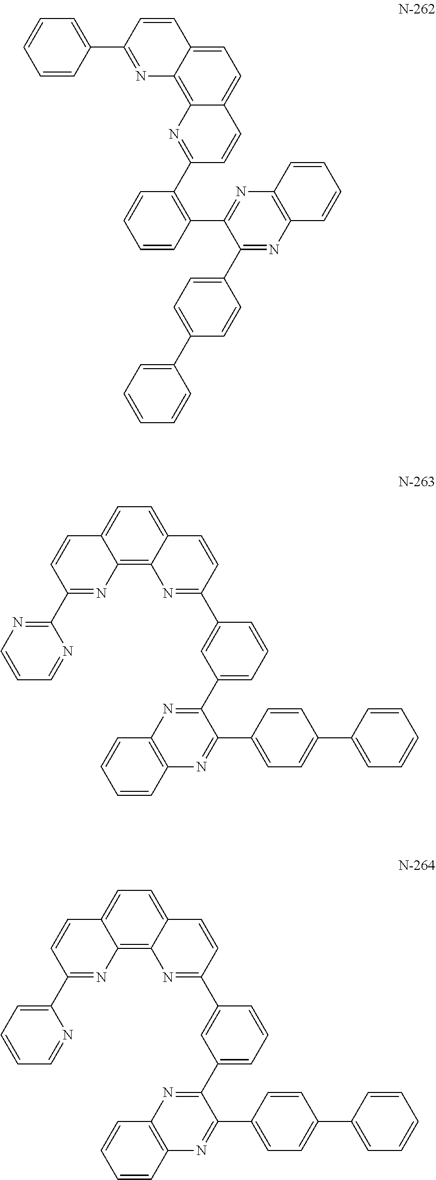 [SMILES: c1ccc(-c2ccc(-c3nc4ccccc4nc3-c3ccccc3-c3ccc4ccc5ccc(-c6ccccc6)nc5c4n3)cc2)cc1 ; c1ccc(-c2ccc(-c3nc4ccccc4nc3-c3cccc(-c4ccc5ccc6ccc(-c7ncccn7)nc6c5n4)c3)cc2)cc1 ; c1ccc(-c2ccc(-c3nc4ccccc4nc3-c3cccc(-c4ccc5ccc6ccc(-c7ccccn7)nc6c5n4)c3)cc2)cc1]

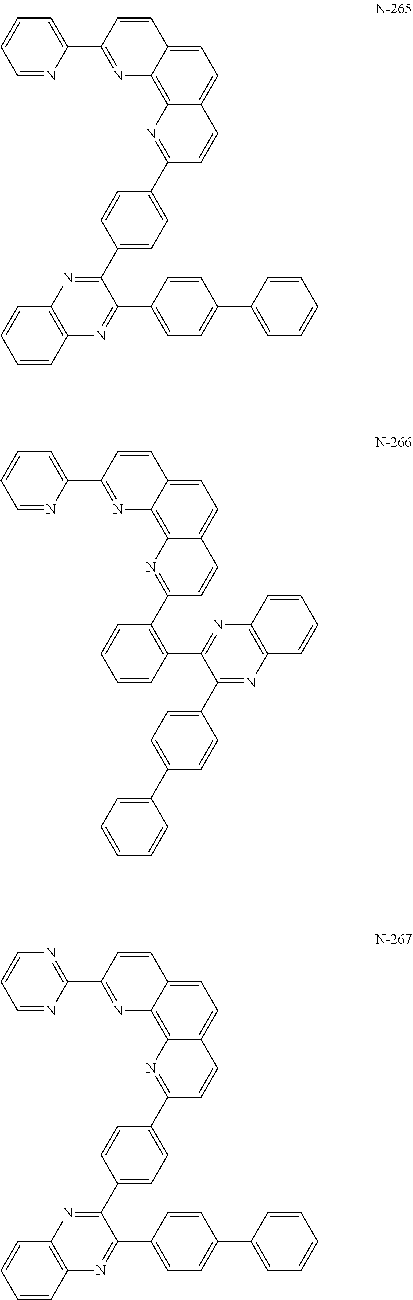 [SMILES: c1ccc(-c2ccc(-c3nc4ccccc4nc3-c3ccc(-c4ccc5ccc6ccc(-c7ccccn7)nc6c5n4)cc3)cc2)cc1 ; c1ccc(-c2ccc(-c3nc4ccccc4nc3-c3ccccc3-c3ccc4ccc5ccc(-c6ccccn6)nc5c4n3)cc2)cc1 ; c1ccc(-c2ccc(-c3nc4ccccc4nc3-c3ccc(-c4ccc5ccc6ccc(-c7ncccn7)nc6c5n4)cc3)cc2)cc1]

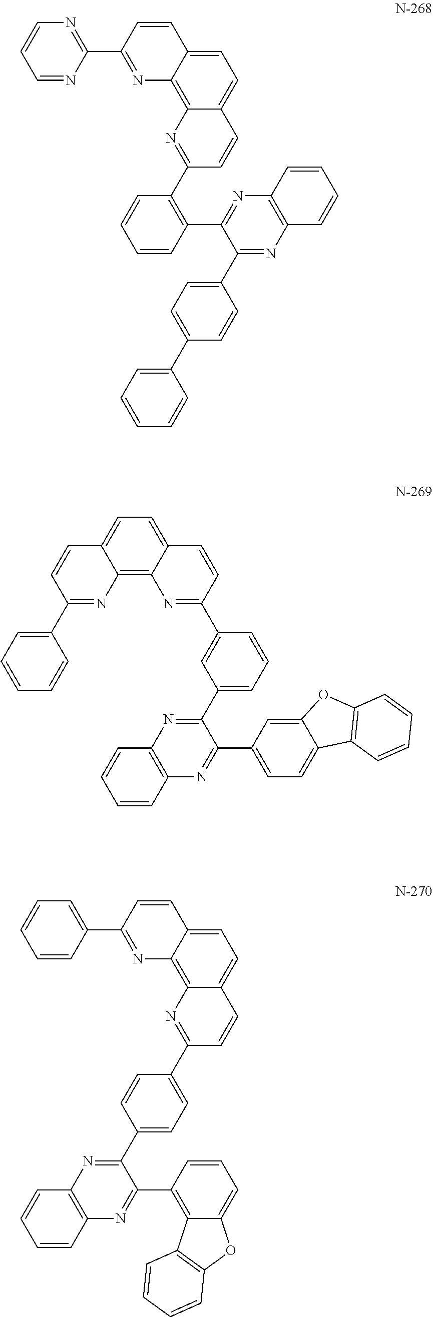 [SMILES: c1ccc(-c2ccc(-c3nc4ccccc4nc3-c3ccccc3-c3ccc4ccc5ccc(-c6ncccn6)nc5c4n3)cc2)cc1 ; c1ccc(-c2ccc3ccc4ccc(-c5cccc(-c6nc7ccccc7nc6-c6ccc7c(c6)oc6ccccc67)c5)nc4c3n2)cc1 ; c1ccc(-c2ccc3ccc4ccc(-c5ccc(-c6nc7ccccc7nc6-c6cccc7oc8ccccc8c67)cc5)nc4c3n2)cc1]

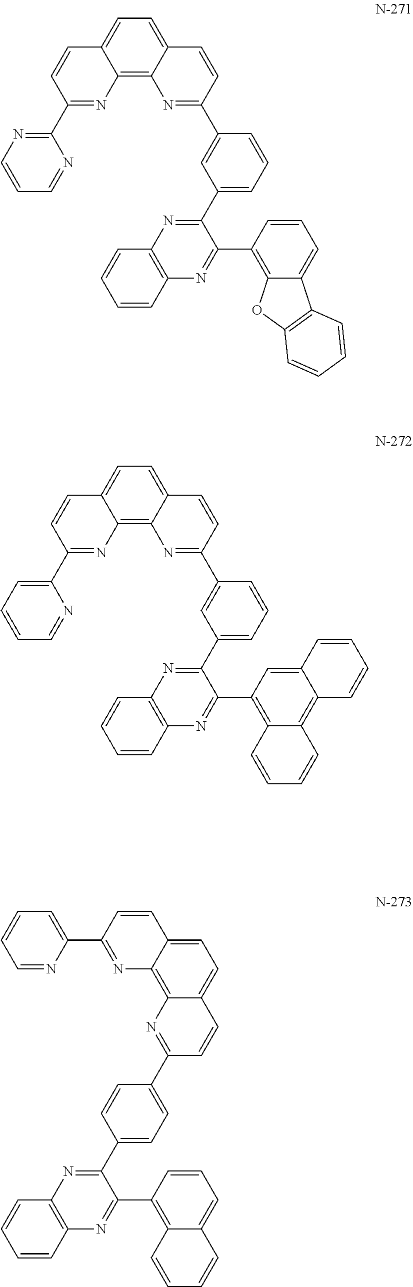 [SMILES: c1cnc(-c2ccc3ccc4ccc(-c5cccc(-c6nc7ccccc7nc6-c6cccc7c6oc6ccccc67)c5)nc4c3n2)nc1 ; c1ccc(-c2ccc3ccc4ccc(-c5cccc(-c6nc7ccccc7nc6-c6cc7ccccc7c7ccccc67)c5)nc4c3n2)nc1 ; c1ccc(-c2ccc3ccc4ccc(-c5ccc(-c6nc7ccccc7nc6-c6cccc7ccccc67)cc5)nc4c3n2)nc1]

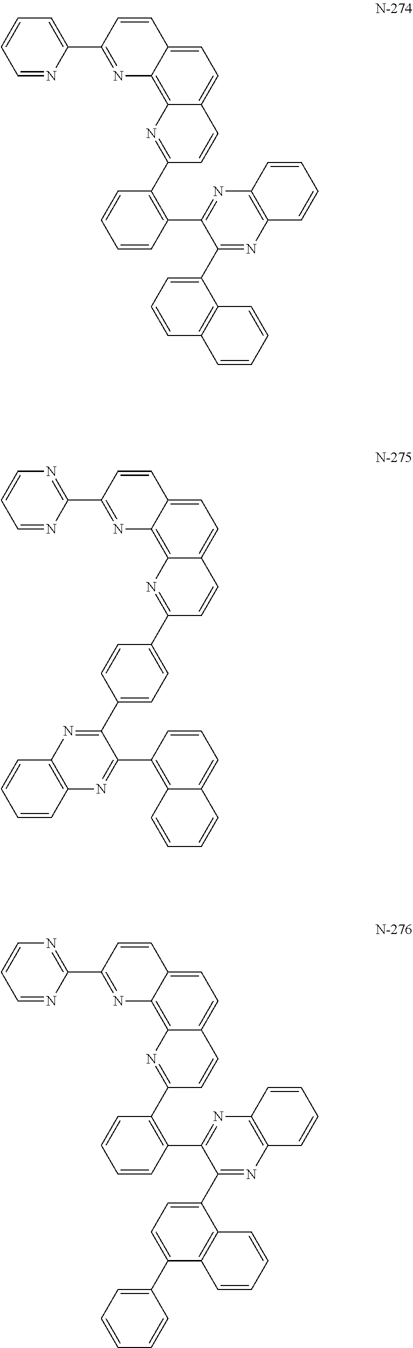 [SMILES: c1ccc(-c2ccc3ccc4ccc(-c5ccccc5-c5nc6ccccc6nc5-c5cccc6ccccc56)nc4c3n2)nc1 ; c1cnc(-c2ccc3ccc4ccc(-c5ccc(-c6nc7ccccc7nc6-c6cccc7ccccc67)cc5)nc4c3n2)nc1 ; c1ccc(-c2ccc(-c3nc4ccccc4nc3-c3ccccc3-c3ccc4ccc5ccc(-c6ncccn6)nc5c4n3)c3ccccc23)cc1]

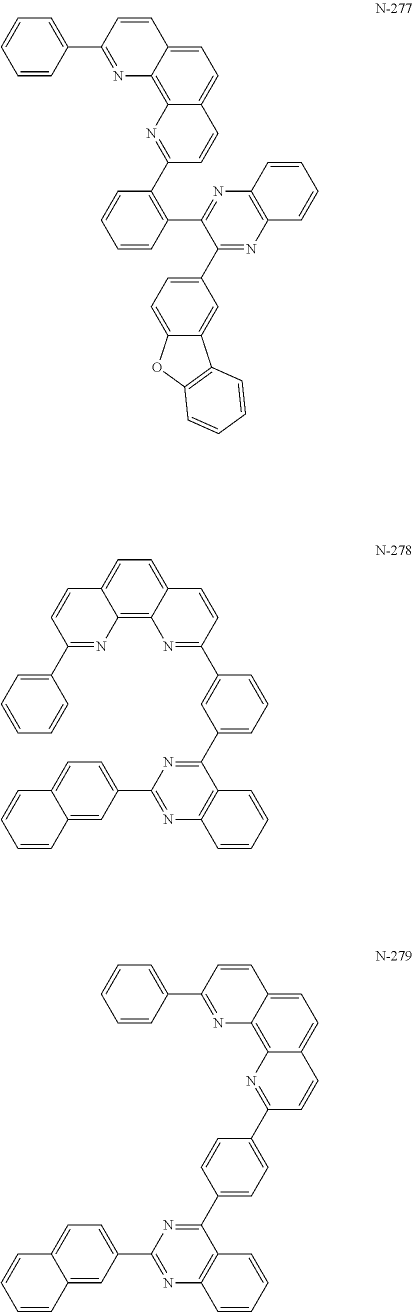 [SMILES: c1ccc(-c2ccc3ccc4ccc(-c5ccccc5-c5nc6ccccc6nc5-c5ccc6oc7ccccc7c6c5)nc4c3n2)cc1 ; c1ccc(-c2ccc3ccc4ccc(-c5cccc(-c6nc(-c7ccc8ccccc8c7)nc7ccccc67)c5)nc4c3n2)cc1 ; c1ccc(-c2ccc3ccc4ccc(-c5ccc(-c6nc(-c7ccc8ccccc8c7)nc7ccccc67)cc5)nc4c3n2)cc1]

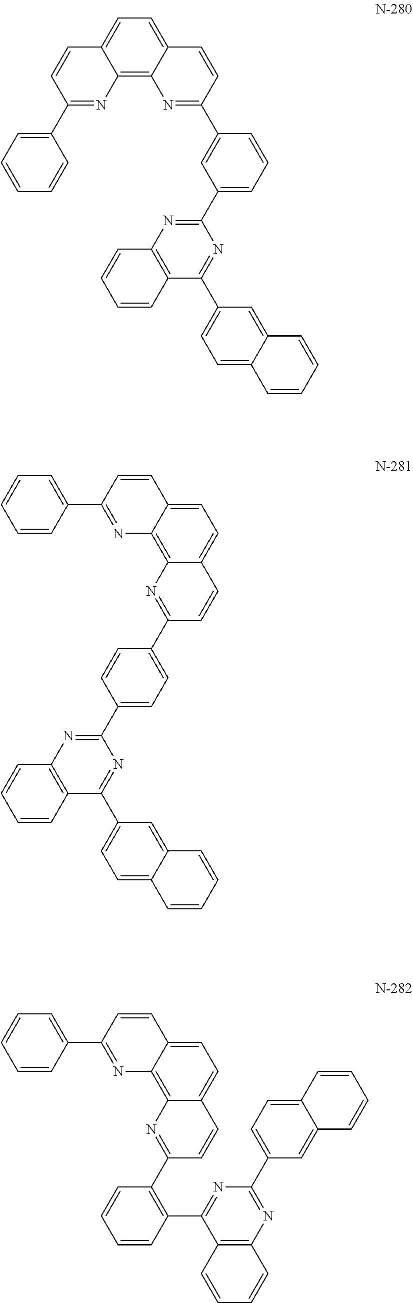 [SMILES: c1ccc(-c2ccc3ccc4ccc(-c5cccc(-c6nc(-c7ccc8ccccc8c7)c7ccccc7n6)c5)nc4c3n2)cc1 ; c1ccc(-c2ccc3ccc4ccc(-c5ccc(-c6nc(-c7ccc8ccccc8c7)c7ccccc7n6)cc5)nc4c3n2)cc1 ; c1ccc(-c2ccc3ccc4ccc(-c5ccccc5-c5nc(-c6ccc7ccccc7c6)nc6ccccc56)nc4c3n2)cc1]

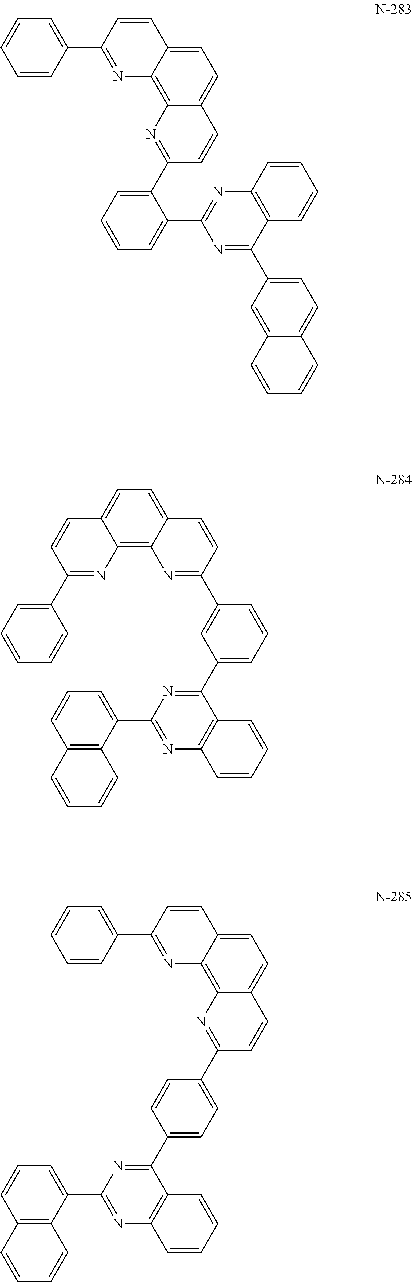 [SMILES: c1ccc(-c2ccc3ccc4ccc(-c5ccccc5-c5nc(-c6ccc7ccccc7c6)c6ccccc6n5)nc4c3n2)cc1 ; c1ccc(-c2ccc3ccc4ccc(-c5cccc(-c6nc(-c7cccc8ccccc78)nc7ccccc67)c5)nc4c3n2)cc1 ; c1ccc(-c2ccc3ccc4ccc(-c5ccc(-c6nc(-c7cccc8ccccc78)nc7ccccc67)cc5)nc4c3n2)cc1]

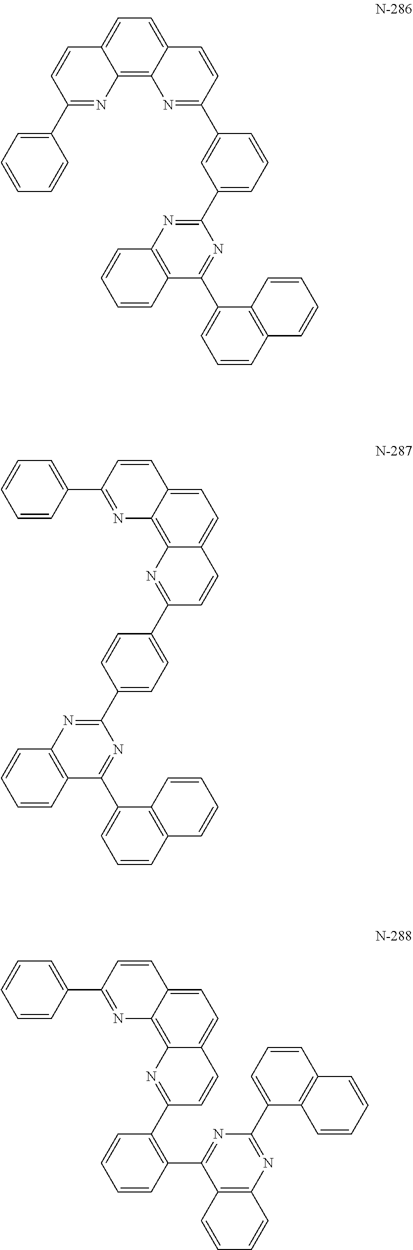 [SMILES: c1ccc(-c2ccc3ccc4ccc(-c5cccc(-c6nc(-c7cccc8ccccc78)c7ccccc7n6)c5)nc4c3n2)cc1 ; c1ccc(-c2ccc3ccc4ccc(-c5ccc(-c6nc(-c7cccc8ccccc78)c7ccccc7n6)cc5)nc4c3n2)cc1 ; c1ccc(-c2ccc3ccc4ccc(-c5ccccc5-c5nc(-c6cccc7ccccc67)nc6ccccc56)nc4c3n2)cc1]

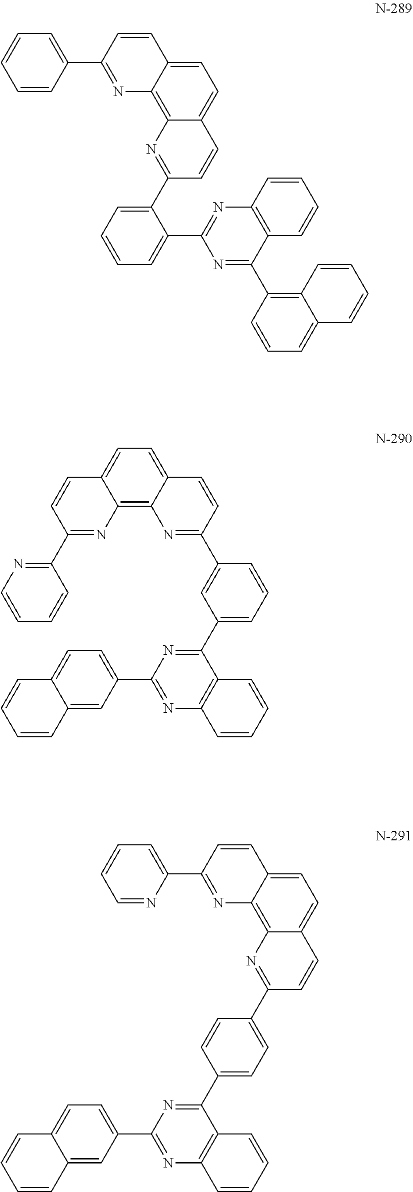 [SMILES: c1ccc(-c2ccc3ccc4ccc(-c5ccccc5-c5nc(-c6cccc7ccccc67)c6ccccc6n5)nc4c3n2)cc1 ; c1ccc(-c2ccc3ccc4ccc(-c5cccc(-c6nc(-c7ccc8ccccc8c7)nc7ccccc67)c5)nc4c3n2)nc1 ; c1ccc(-c2ccc3ccc4ccc(-c5ccc(-c6nc(-c7ccc8ccccc8c7)nc7ccccc67)cc5)nc4c3n2)nc1]

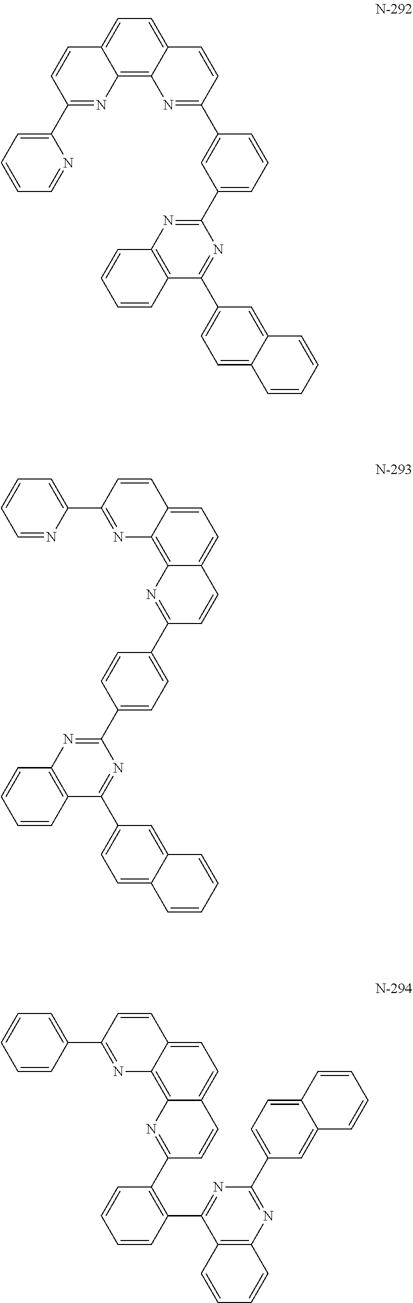 [SMILES: c1ccc(-c2ccc3ccc4ccc(-c5cccc(-c6nc(-c7ccc8ccccc8c7)c7ccccc7n6)c5)nc4c3n2)nc1 ; c1ccc(-c2ccc3ccc4ccc(-c5ccc(-c6nc(-c7ccc8ccccc8c7)c7ccccc7n6)cc5)nc4c3n2)nc1 ; c1ccc(-c2ccc3ccc4ccc(-c5ccccc5-c5nc(-c6ccc7ccccc7c6)nc6ccccc56)nc4c3n2)cc1]

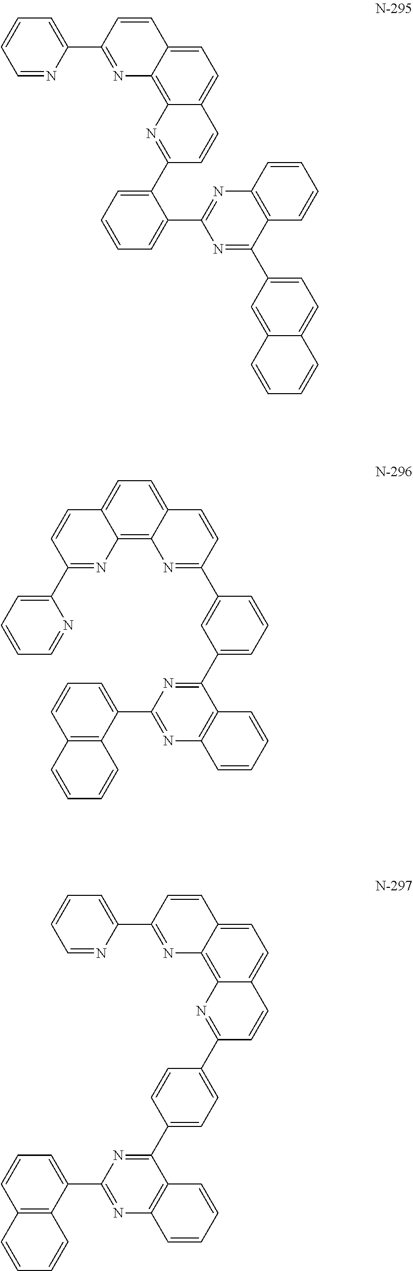 [SMILES: c1ccc(-c2ccc3ccc4ccc(-c5ccccc5-c5nc(-c6ccc7ccccc7c6)c6ccccc6n5)nc4c3n2)nc1 ; c1ccc(-c2ccc3ccc4ccc(-c5cccc(-c6nc(-c7cccc8ccccc78)nc7ccccc67)c5)nc4c3n2)nc1 ; c1ccc(-c2ccc3ccc4ccc(-c5ccc(-c6nc(-c7cccc8ccccc78)nc7ccccc67)cc5)nc4c3n2)nc1]

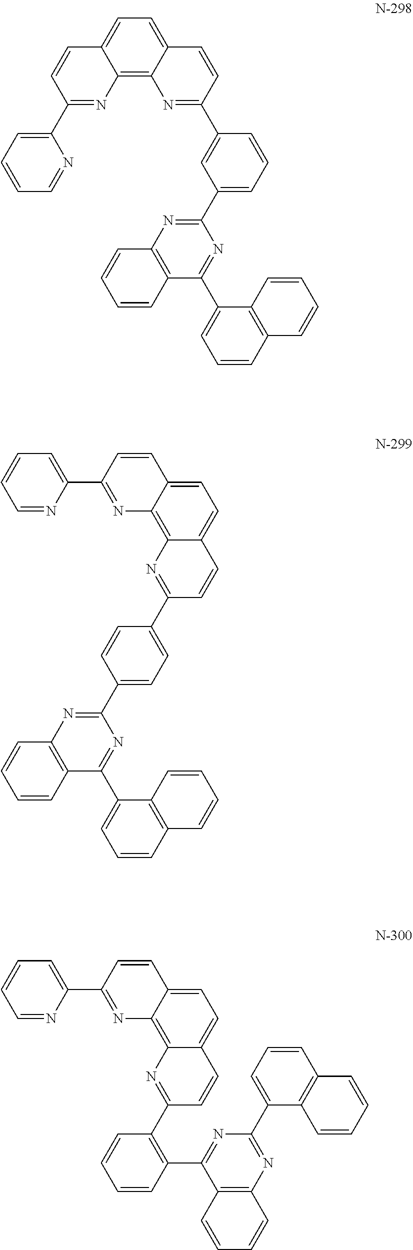 [SMILES: c1ccc(-c2ccc3ccc4ccc(-c5cccc(-c6nc(-c7cccc8ccccc78)c7ccccc7n6)c5)nc4c3n2)nc1 ; c1ccc(-c2ccc3ccc4ccc(-c5ccc(-c6nc(-c7cccc8ccccc78)c7ccccc7n6)cc5)nc4c3n2)nc1 ; c1ccc(-c2ccc3ccc4ccc(-c5ccccc5-c5nc(-c6cccc7ccccc67)nc6ccccc56)nc4c3n2)nc1]

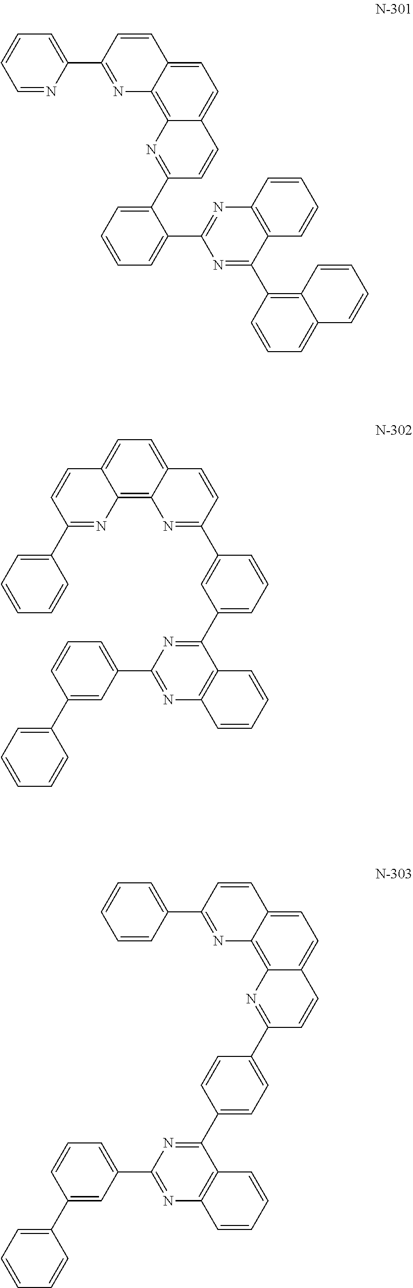 [SMILES: c1ccc(-c2ccc3ccc4ccc(-c5ccccc5-c5nc(-c6cccc7ccccc67)c6ccccc6n5)nc4c3n2)nc1 ; c1ccc(-c2cccc(-c3nc(-c4cccc(-c5ccc6ccc7ccc(-c8ccccc8)nc7c6n5)c4)c4ccccc4n3)c2)cc1 ; c1ccc(-c2cccc(-c3nc(-c4ccc(-c5ccc6ccc7ccc(-c8ccccc8)nc7c6n5)cc4)c4ccccc4n3)c2)cc1]

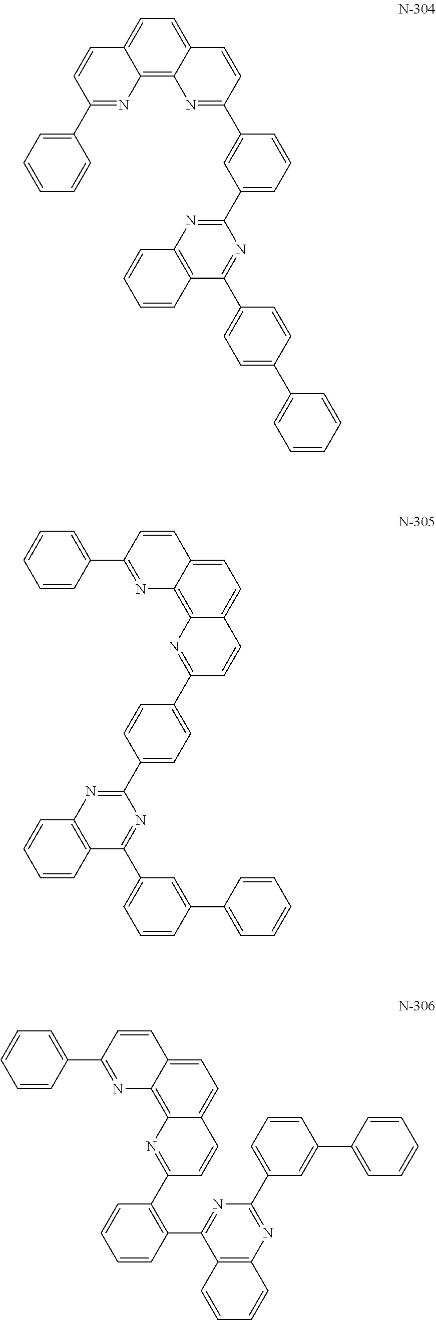 [SMILES: c1ccc(-c2ccc(-c3nc(-c4cccc(-c5ccc6ccc7ccc(-c8ccccc8)nc7c6n5)c4)nc4ccccc34)cc2)cc1 ; c1ccc(-c2cccc(-c3nc(-c4ccc(-c5ccc6ccc7ccc(-c8ccccc8)nc7c6n5)cc4)nc4ccccc34)c2)cc1 ; c1ccc(-c2cccc(-c3nc(-c4ccccc4-c4ccc5ccc6ccc(-c7ccccc7)nc6c5n4)c4ccccc4n3)c2)cc1]

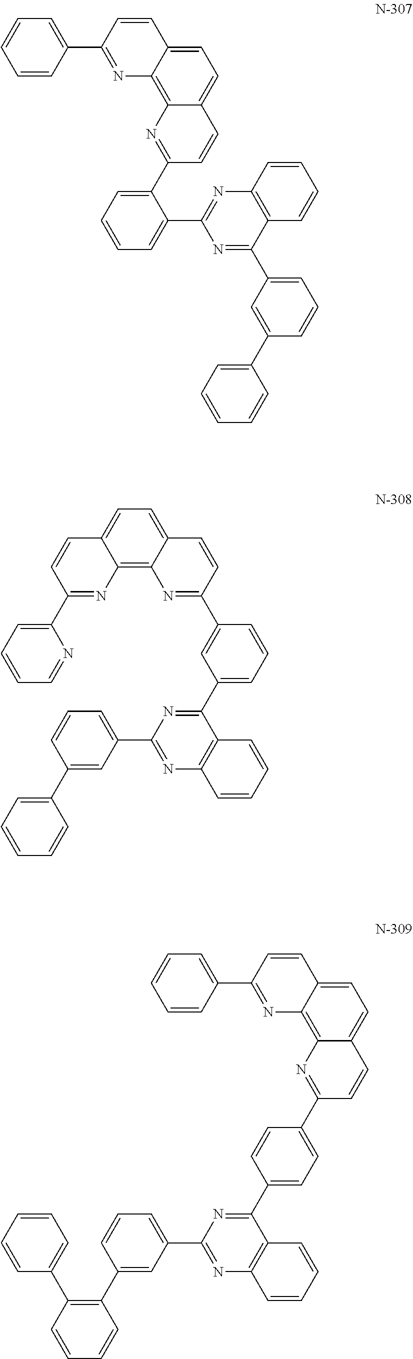 [SMILES: c1ccc(-c2cccc(-c3nc(-c4ccccc4-c4ccc5ccc6ccc(-c7ccccc7)nc6c5n4)nc4ccccc34)c2)cc1 ; c1ccc(-c2cccc(-c3nc(-c4cccc(-c5ccc6ccc7ccc(-c8ccccn8)nc7c6n5)c4)c4ccccc4n3)c2)cc1 ; c1ccc(-c2ccc3ccc4ccc(-c5ccc(-c6nc(-c7cccc(-c8ccccc8-c8ccccc8)c7)nc7ccccc67)cc5)nc4c3n2)cc1]

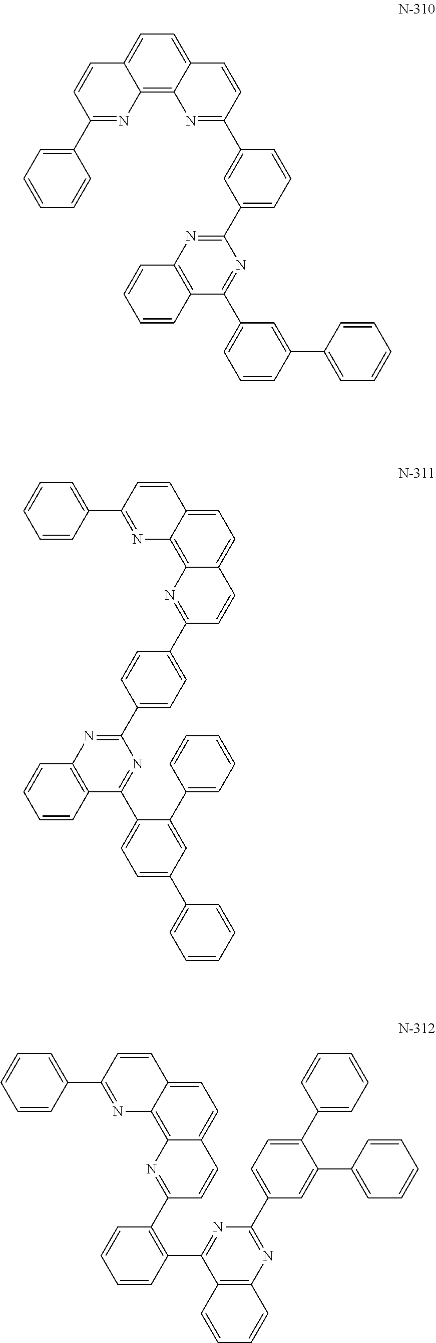 [SMILES: c1ccc(-c2cccc(-c3nc(-c4cccc(-c5ccc6ccc7ccc(-c8ccccc8)nc7c6n5)c4)nc4ccccc34)c2)cc1 ; c1ccc(-c2ccc(-c3nc(-c4ccc(-c5ccc6ccc7ccc(-c8ccccc8)nc7c6n5)cc4)nc4ccccc34)c(-c3ccccc3)c2)cc1 ; c1ccc(-c2ccc3ccc4ccc(-c5ccccc5-c5nc(-c6ccc(-c7ccccc7)c(-c7ccccc7)c6)nc6ccccc56)nc4c3n2)cc1]

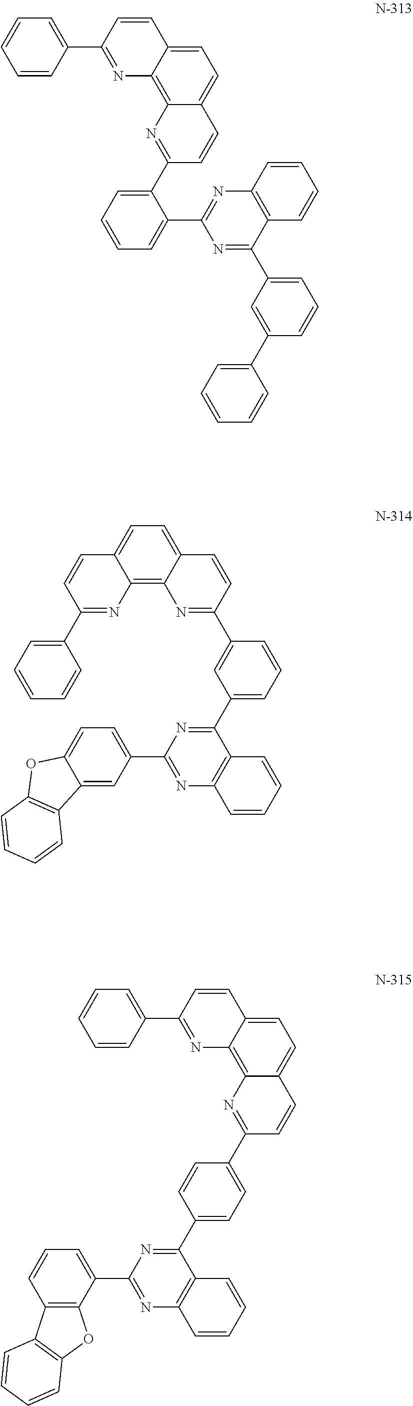 [SMILES: c1ccc(-c2cccc(-c3nc(-c4ccccc4-c4ccc5ccc6ccc(-c7ccccc7)nc6c5n4)nc4ccccc34)c2)cc1 ; c1ccc(-c2ccc3ccc4ccc(-c5cccc(-c6nc(-c7ccc8oc9ccccc9c8c7)nc7ccccc67)c5)nc4c3n2)cc1 ; c1ccc(-c2ccc3ccc4ccc(-c5ccc(-c6nc(-c7cccc8c7oc7ccccc78)nc7ccccc67)cc5)nc4c3n2)cc1]

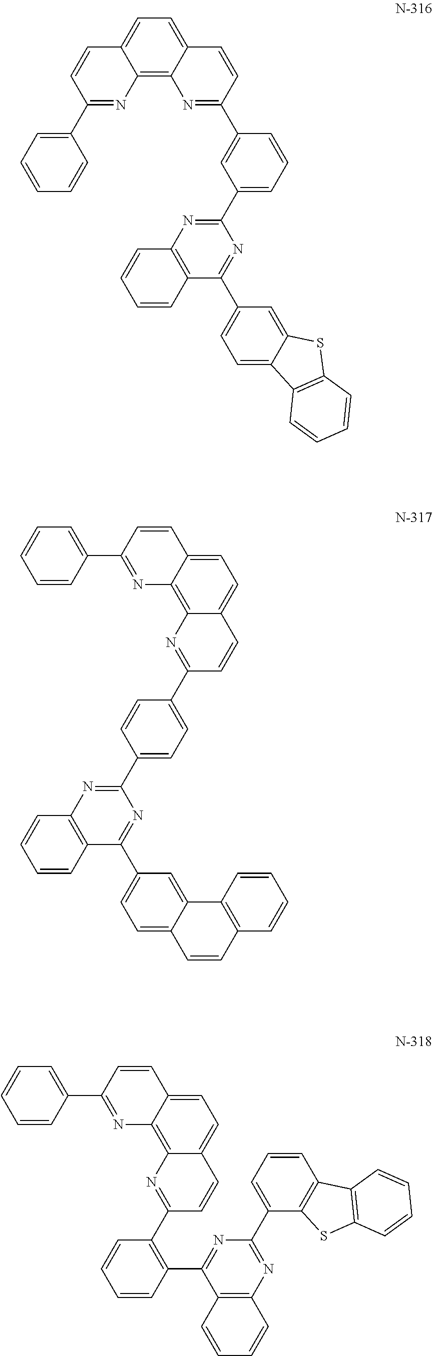 [SMILES: c1ccc(-c2ccc3ccc4ccc(-c5cccc(-c6nc(-c7ccc8c(c7)sc7ccccc78)c7ccccc7n6)c5)nc4c3n2)cc1 ; c1ccc(-c2ccc3ccc4ccc(-c5ccc(-c6nc(-c7ccc8ccc9ccccc9c8c7)c7ccccc7n6)cc5)nc4c3n2)cc1 ; c1ccc(-c2ccc3ccc4ccc(-c5ccccc5-c5nc(-c6cccc7c6sc6ccccc67)nc6ccccc56)nc4c3n2)cc1]

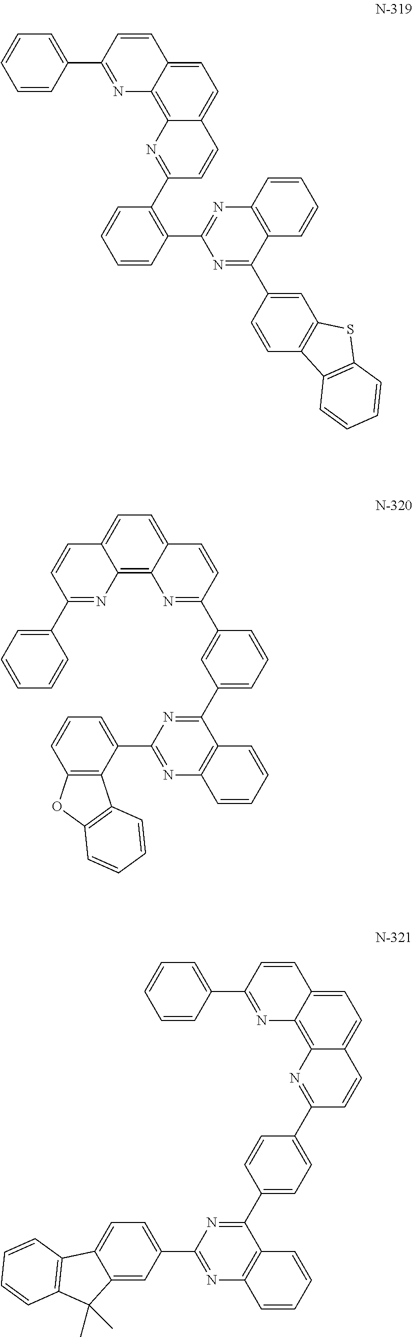 [SMILES: c1ccc(-c2ccc3ccc4ccc(-c5ccccc5-c5nc(-c6ccc7c(c6)sc6ccccc67)c6ccccc6n5)nc4c3n2)cc1 ; c1ccc(-c2ccc3ccc4ccc(-c5cccc(-c6nc(-c7cccc8oc9ccccc9c78)nc7ccccc67)c5)nc4c3n2)cc1 ; CC1(C)c2ccccc2-c2ccc(-c3nc(-c4ccc(-c5ccc6ccc7ccc(-c8ccccc8)nc7c6n5)cc4)c4ccccc4n3)cc21]

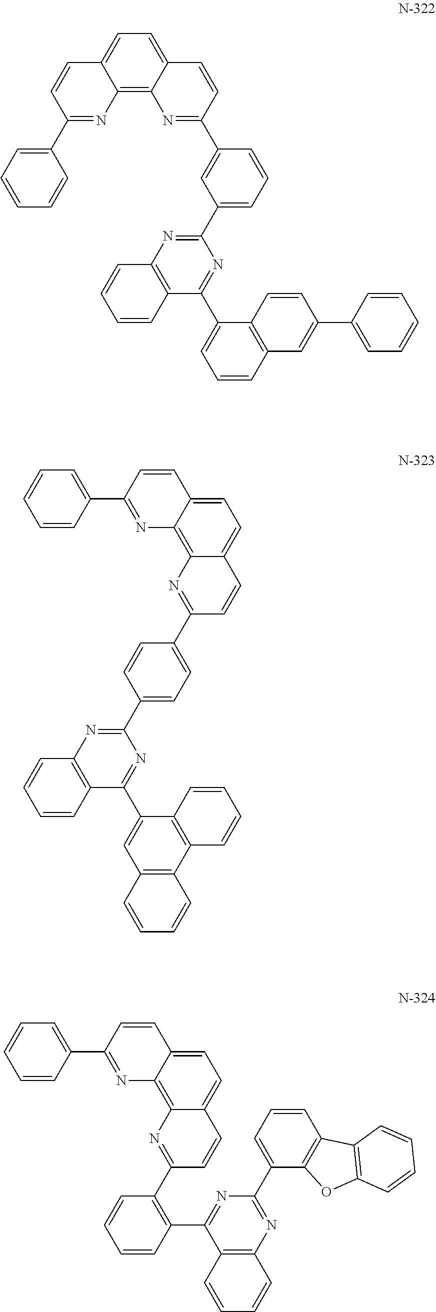 [SMILES: c1ccc(-c2ccc3c(-c4nc(-c5cccc(-c6ccc7ccc8ccc(-c9ccccc9)nc8c7n6)c5)nc5ccccc45)cccc3c2)cc1 ; c1ccc(-c2ccc3ccc4ccc(-c5ccc(-c6nc(-c7cc8ccccc8c8ccccc78)c7ccccc7n6)cc5)nc4c3n2)cc1 ; c1ccc(-c2ccc3ccc4ccc(-c5ccccc5-c5nc(-c6cccc7c6oc6ccccc67)nc6ccccc56)nc4c3n2)cc1]

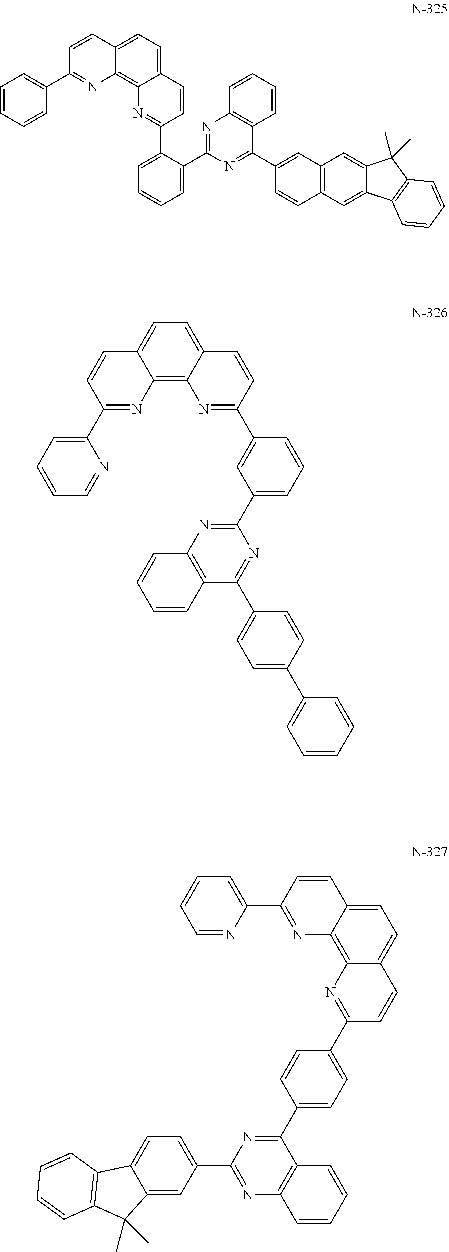 [SMILES: CC1(C)c2ccccc2-c2cc3ccc(-c4nc(-c5ccccc5-c5ccc6ccc7ccc(-c8ccccc8)nc7c6n5)nc5ccccc45)cc3cc21 ; c1ccc(-c2ccc(-c3nc(-c4cccc(-c5ccc6ccc7ccc(-c8ccccn8)nc7c6n5)c4)nc4ccccc34)cc2)cc1 ; CC1(C)c2ccccc2-c2ccc(-c3nc(-c4ccc(-c5ccc6ccc7ccc(-c8ccccn8)nc7c6n5)cc4)c4ccccc4n3)cc21]

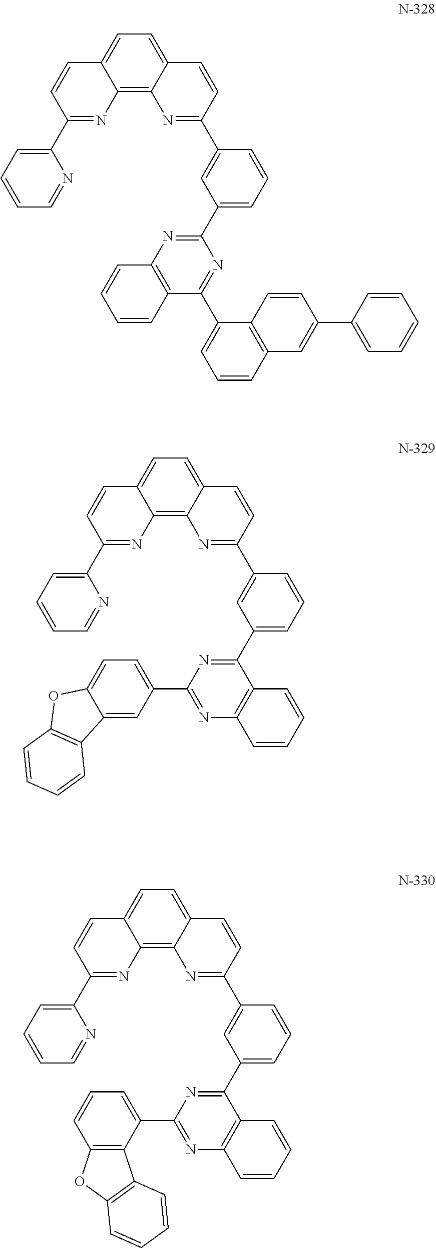 [SMILES: c1ccc(-c2ccc3c(-c4nc(-c5cccc(-c6ccc7ccc8ccc(-c9ccccn9)nc8c7n6)c5)nc5ccccc45)cccc3c2)cc1 ; c1ccc(-c2ccc3ccc4ccc(-c5cccc(-c6nc(-c7ccc8oc9ccccc9c8c7)nc7ccccc67)c5)nc4c3n2)nc1 ; c1ccc(-c2ccc3ccc4ccc(-c5cccc(-c6nc(-c7cccc8oc9ccccc9c78)nc7ccccc67)c5)nc4c3n2)nc1]

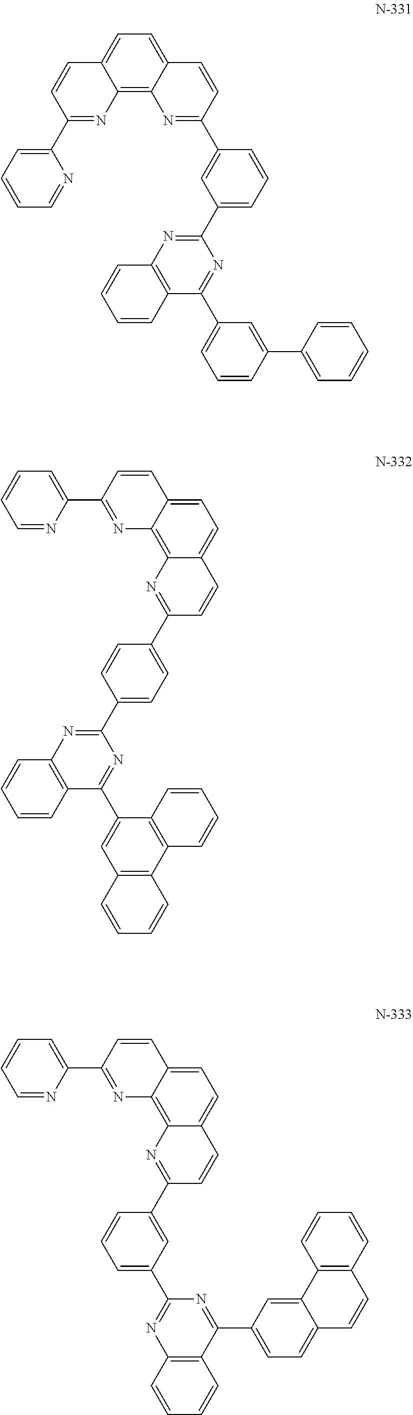 [SMILES: c1ccc(-c2cccc(-c3nc(-c4cccc(-c5ccc6ccc7ccc(-c8ccccn8)nc7c6n5)c4)nc4ccccc34)c2)cc1 ; c1ccc(-c2ccc3ccc4ccc(-c5ccc(-c6nc(-c7cc8ccccc8c8ccccc78)c7ccccc7n6)cc5)nc4c3n2)nc1 ; c1ccc(-c2ccc3ccc4ccc(-c5cccc(-c6nc(-c7ccc8ccc9ccccc9c8c7)c7ccccc7n6)c5)nc4c3n2)nc1]

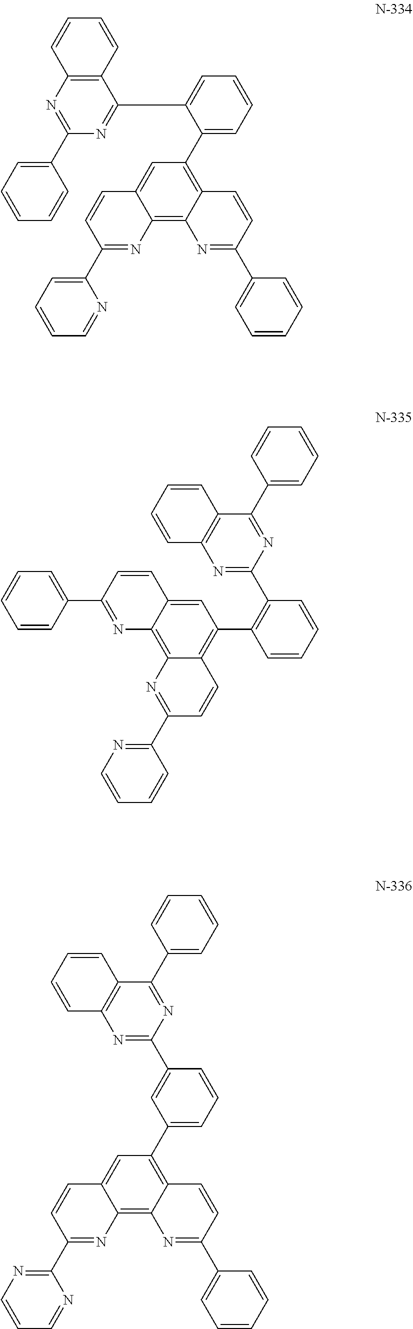 [SMILES: c1ccc(-c2ccc3c(-c4ccccc4-c4nc(-c5ccccc5)nc5ccccc45)cc4ccc(-c5ccccn5)nc4c3n2)cc1 ; c1ccc(-c2ccc3cc(-c4ccccc4-c4nc(-c5ccccc5)c5ccccc5n4)c4ccc(-c5ccccn5)nc4c3n2)cc1 ; c1ccc(-c2ccc3c(-c4cccc(-c5nc(-c6ccccc6)c6ccccc6n5)c4)cc4ccc(-c5ncccn5)nc4c3n2)cc1]

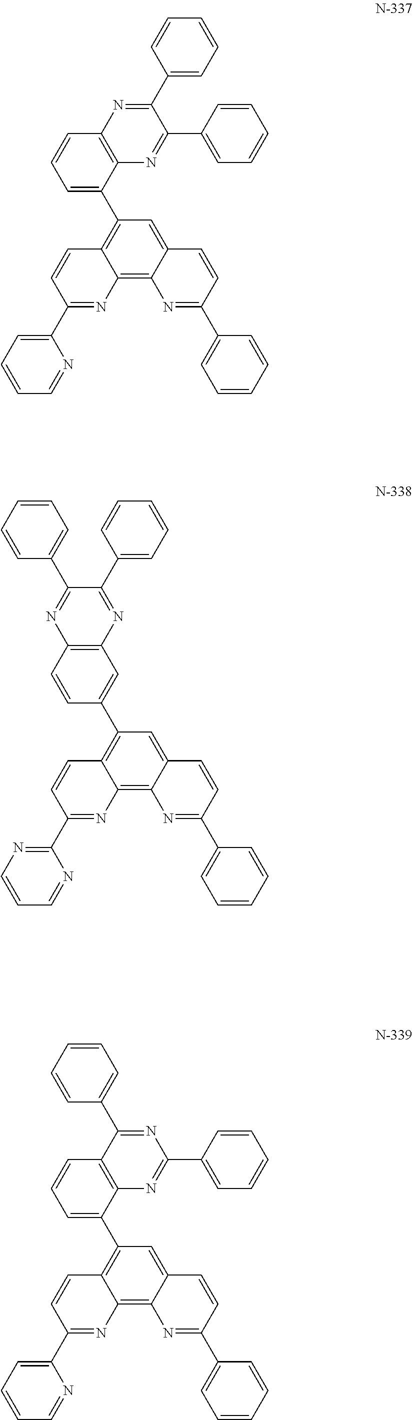 [SMILES: c1ccc(-c2ccc3cc(-c4cccc5nc(-c6ccccc6)c(-c6ccccc6)nc45)c4ccc(-c5ccccn5)nc4c3n2)cc1 ; c1ccc(-c2ccc3cc(-c4ccc5nc(-c6ccccc6)c(-c6ccccc6)nc5c4)c4ccc(-c5ncccn5)nc4c3n2)cc1 ; c1ccc(-c2ccc3cc(-c4cccc5c(-c6ccccc6)nc(-c6ccccc6)nc45)c4ccc(-c5ccccn5)nc4c3n2)cc1]

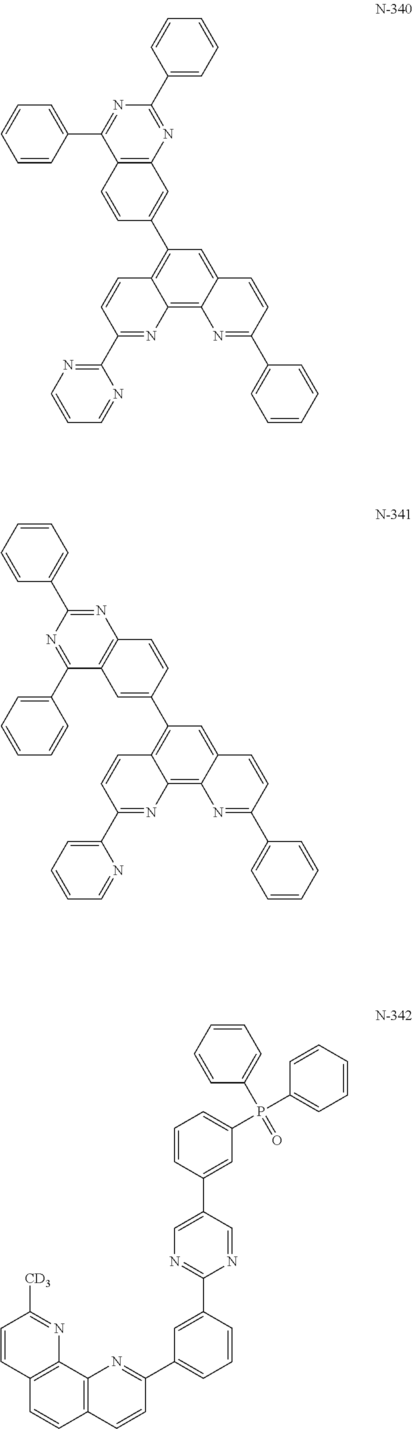 [SMILES: c1ccc(-c2ccc3cc(-c4ccc5c(-c6ccccc6)nc(-c6ccccc6)nc5c4)c4ccc(-c5ncccn5)nc4c3n2)cc1 ; c1ccc(-c2ccc3cc(-c4ccc5nc(-c6ccccc6)nc(-c6ccccc6)c5c4)c4ccc(-c5ccccn5)nc4c3n2)cc1 ; [2H]C([2H])([2H])c1ccc2ccc3ccc(-c4cccc(-c5ncc(-c6cccc(P(=O)(c7ccccc7)c7ccccc7)c6)cn5)c4)nc3c2n1]

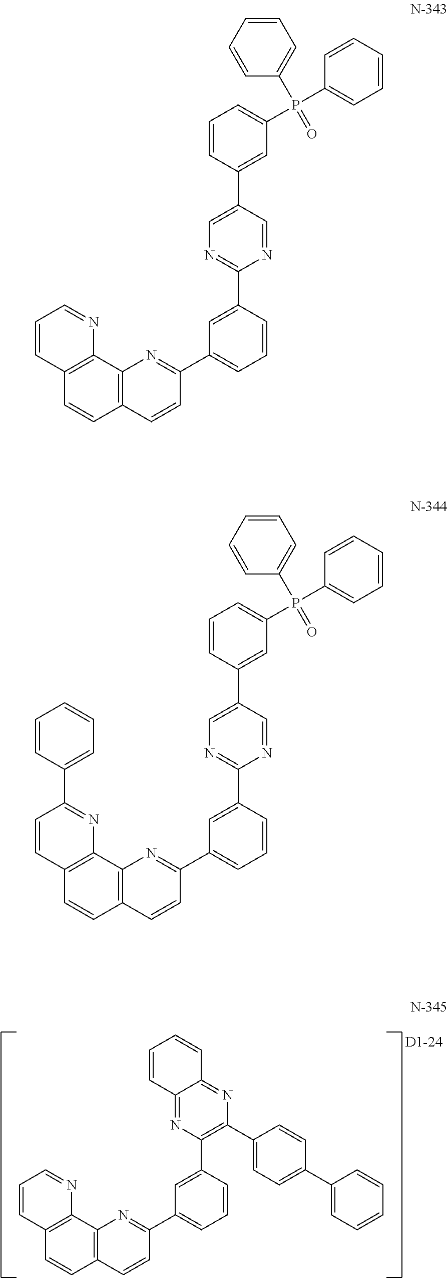

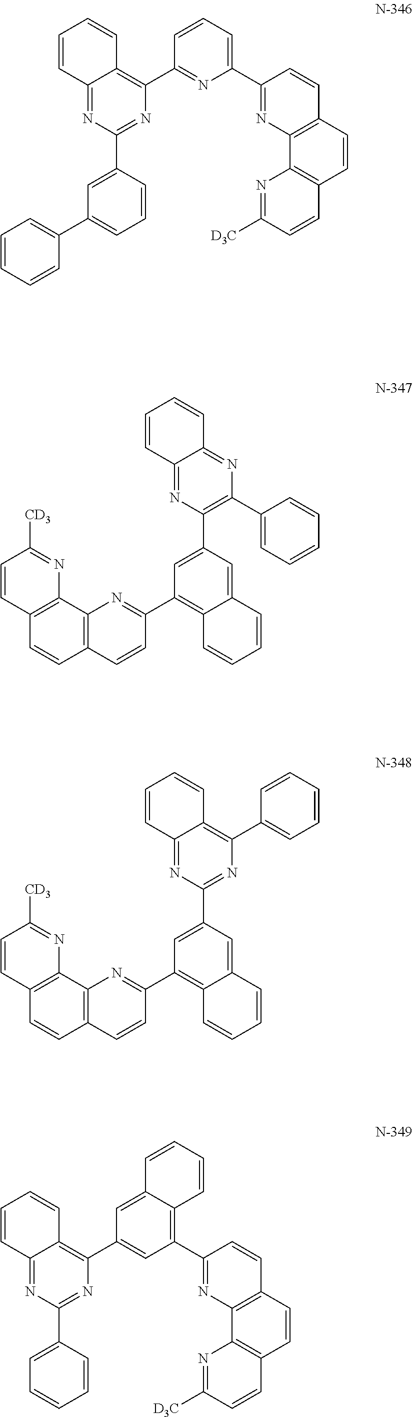 [SMILES: [2H]C([2H])([2H])c1ccc2ccc3ccc(-c4cccc(-c5nc(-c6cccc(-c7ccccc7)c6)nc6ccccc56)n4)nc3c2n1 ; [2H]C([2H])([2H])c1ccc2ccc3ccc(-c4cc(-c5nc6ccccc6nc5-c5ccccc5)cc5ccccc45)nc3c2n1 ; [2H]C([2H])([2H])c1ccc2ccc3ccc(-c4cc(-c5nc(-c6ccccc6)c6ccccc6n5)cc5ccccc45)nc3c2n1 ; [2H]C([2H])([2H])c1ccc2ccc3ccc(-c4cc(-c5nc(-c6ccccc6)nc6ccccc56)cc5ccccc45)nc3c2n1]

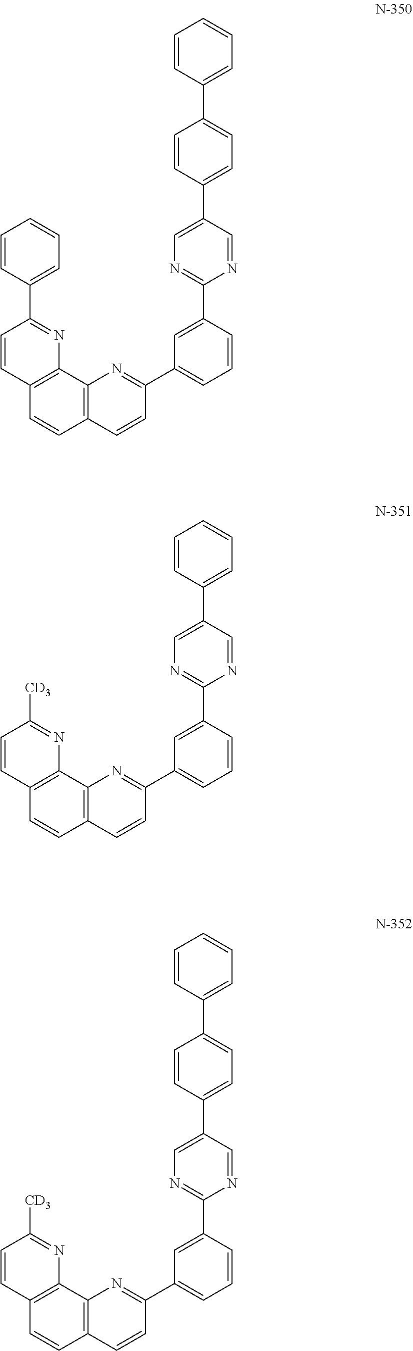 [SMILES: c1ccc(-c2ccc(-c3cnc(-c4cccc(-c5ccc6ccc7ccc(-c8ccccc8)nc7c6n5)c4)nc3)cc2)cc1 ; [2H]C([2H])([2H])c1ccc2ccc3ccc(-c4cccc(-c5ncc(-c6ccccc6)cn5)c4)nc3c2n1 ; [2H]C([2H])([2H])c1ccc2ccc3ccc(-c4cccc(-c5ncc(-c6ccc(-c7ccccc7)cc6)cn5)c4)nc3c2n1]

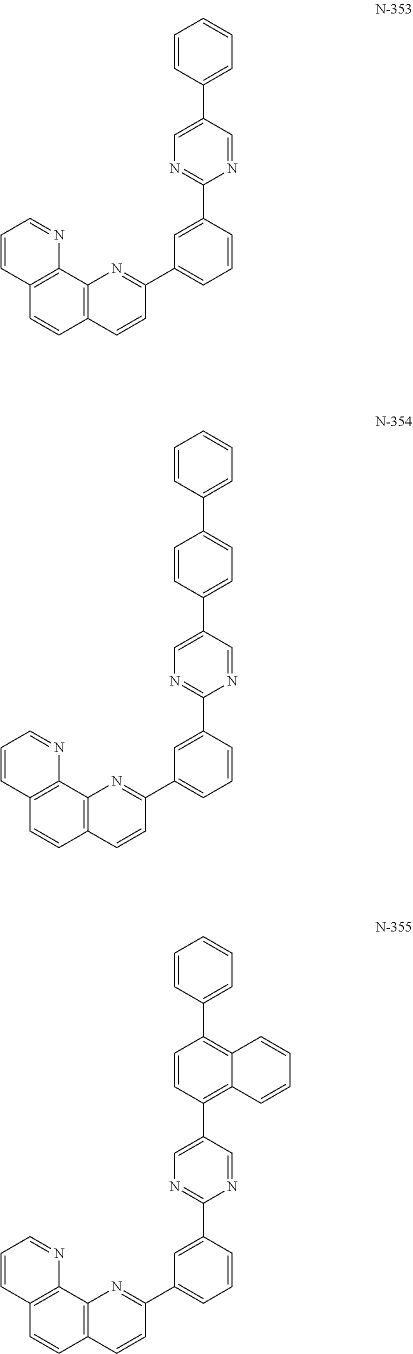 [SMILES: c1ccc(-c2cnc(-c3cccc(-c4ccc5ccc6cccnc6c5n4)c3)nc2)cc1 ; c1ccc(-c2ccc(-c3cnc(-c4cccc(-c5ccc6ccc7cccnc7c6n5)c4)nc3)cc2)cc1 ; c1ccc(-c2ccc(-c3cnc(-c4cccc(-c5ccc6ccc7cccnc7c6n5)c4)nc3)c3ccccc23)cc1]

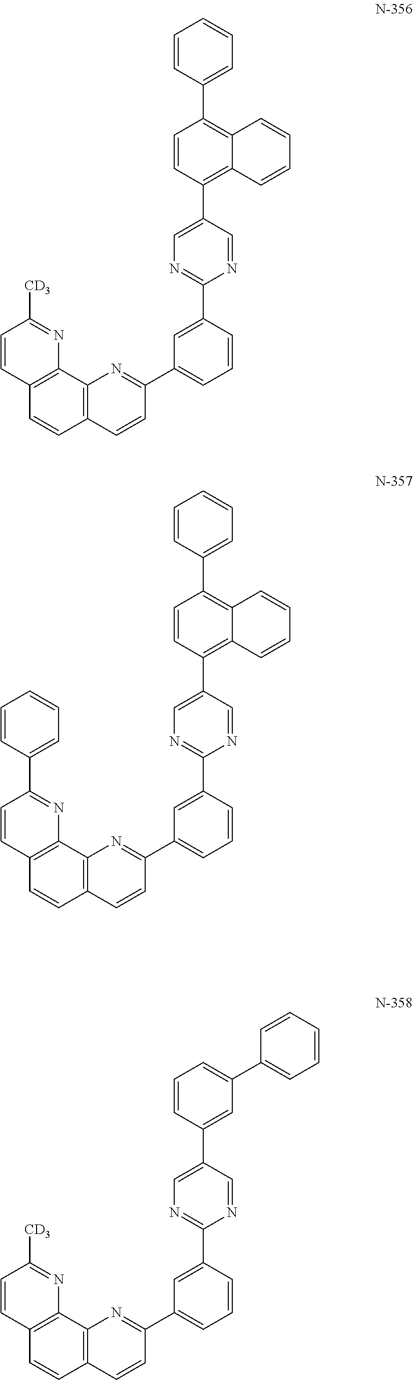 [SMILES: [2H]C([2H])([2H])c1ccc2ccc3ccc(-c4cccc(-c5ncc(-c6ccc(-c7ccccc7)c7ccccc67)cn5)c4)nc3c2n1 ; c1ccc(-c2ccc3ccc4ccc(-c5cccc(-c6ncc(-c7ccc(-c8ccccc8)c8ccccc78)cn6)c5)nc4c3n2)cc1 ; [2H]C([2H])([2H])c1ccc2ccc3ccc(-c4cccc(-c5ncc(-c6cccc(-c7ccccc7)c6)cn5)c4)nc3c2n1]

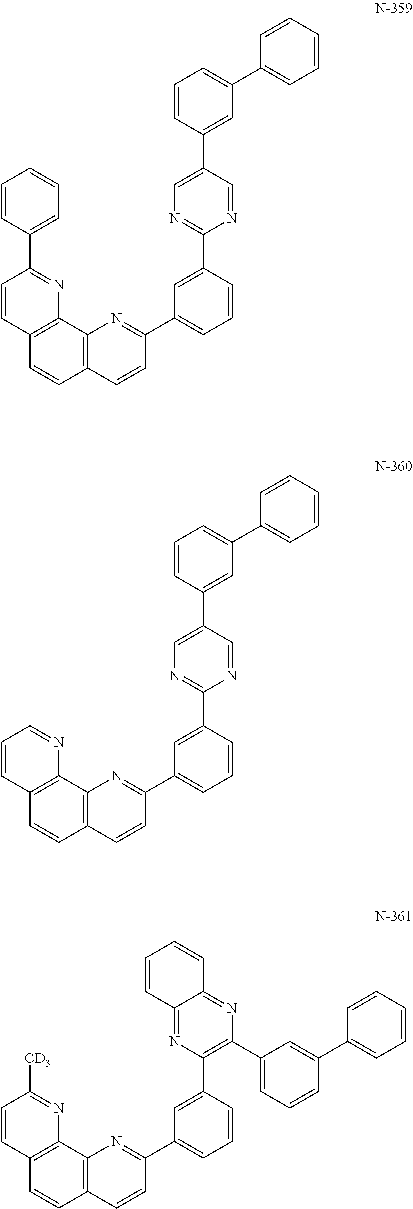 [SMILES: c1ccc(-c2cccc(-c3cnc(-c4cccc(-c5ccc6ccc7ccc(-c8ccccc8)nc7c6n5)c4)nc3)c2)cc1 ; c1ccc(-c2cccc(-c3cnc(-c4cccc(-c5ccc6ccc7cccnc7c6n5)c4)nc3)c2)cc1 ; [2H]C([2H])([2H])c1ccc2ccc3ccc(-c4cccc(-c5nc6ccccc6nc5-c5cccc(-c6ccccc6)c5)c4)nc3c2n1]

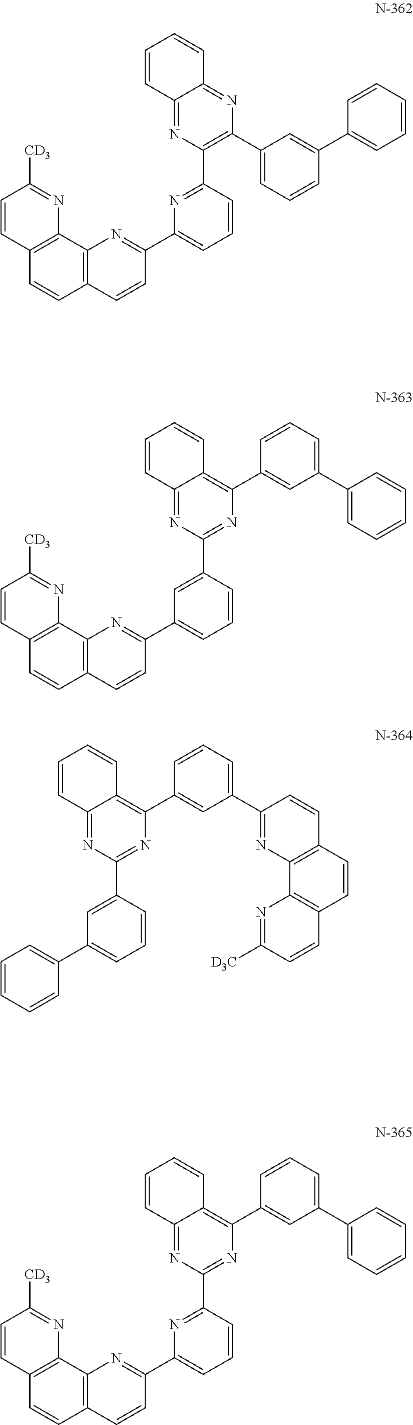 [SMILES: [2H]C([2H])([2H])c1ccc2ccc3ccc(-c4cccc(-c5nc6ccccc6nc5-c5cccc(-c6ccccc6)c5)n4)nc3c2n1 ; [2H]C([2H])([2H])c1ccc2ccc3ccc(-c4cccc(-c5nc(-c6cccc(-c7ccccc7)c6)c6ccccc6n5)c4)nc3c2n1 ; [2H]C([2H])([2H])c1ccc2ccc3ccc(-c4cccc(-c5nc(-c6cccc(-c7ccccc7)c6)nc6ccccc56)c4)nc3c2n1 ; [2H]C([2H])([2H])c1ccc2ccc3ccc(-c4cccc(-c5nc(-c6cccc(-c7ccccc7)c6)c6ccccc6n5)n4)nc3c2n1]

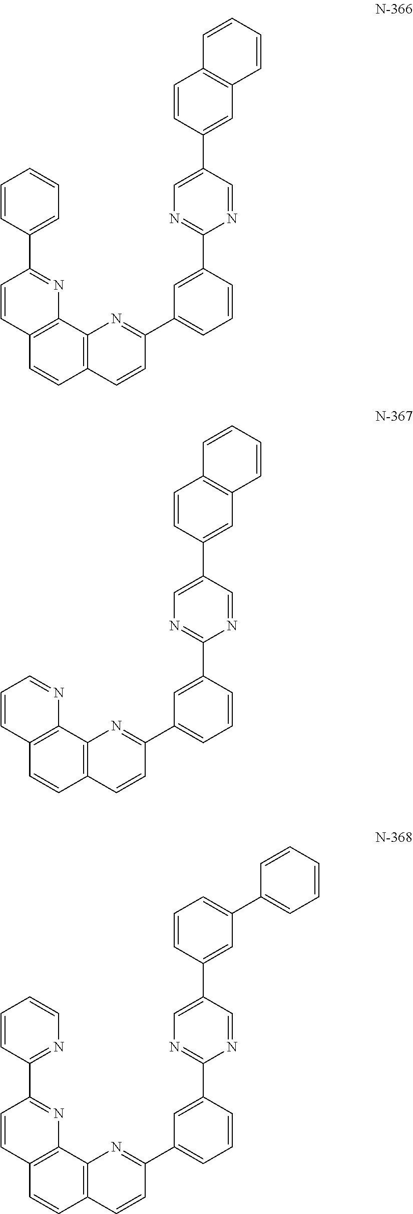 [SMILES: c1ccc(-c2ccc3ccc4ccc(-c5cccc(-c6ncc(-c7ccc8ccccc8c7)cn6)c5)nc4c3n2)cc1 ; c1cc(-c2ccc3ccc4cccnc4c3n2)cc(-c2ncc(-c3ccc4ccccc4c3)cn2)c1 ; c1ccc(-c2cccc(-c3cnc(-c4cccc(-c5ccc6ccc7ccc(-c8ccccn8)nc7c6n5)c4)nc3)c2)cc1]

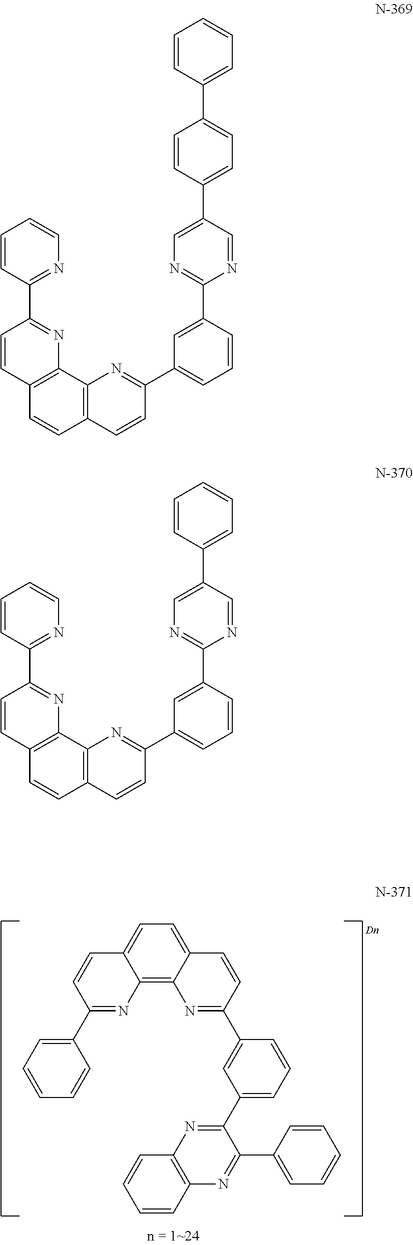

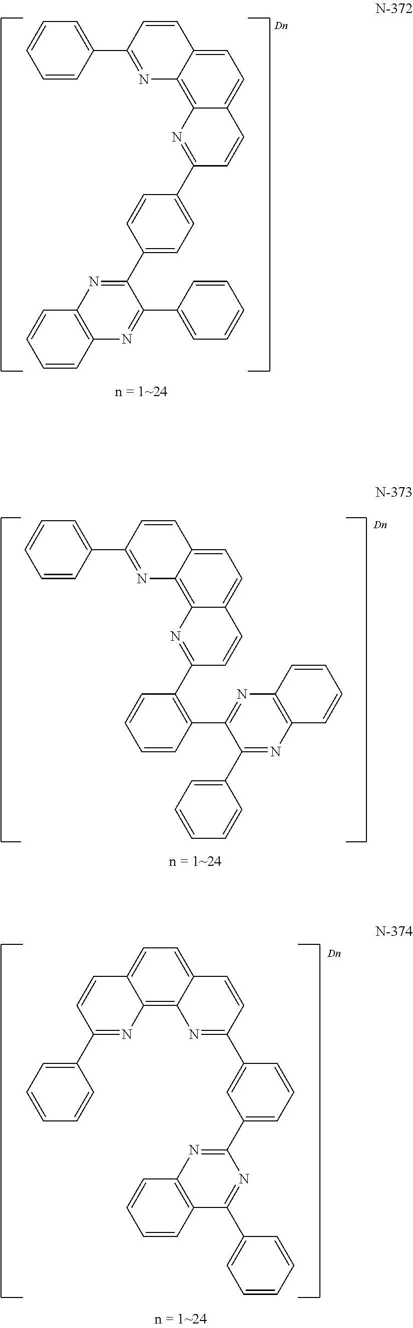

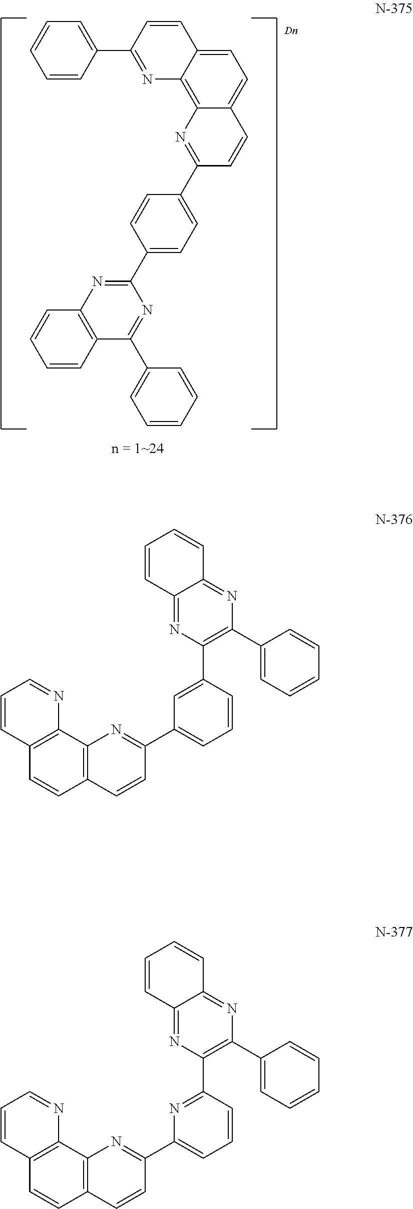

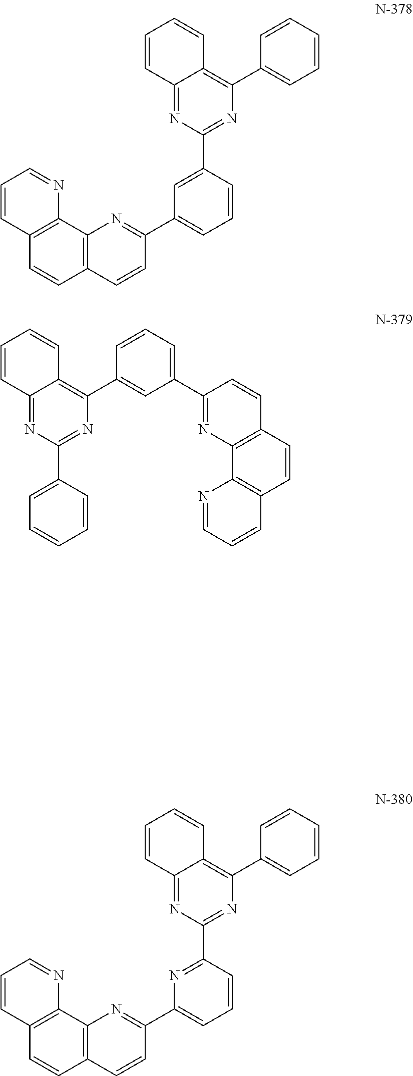 [SMILES: c1ccc(-c2nc(-c3cccc(-c4ccc5ccc6cccnc6c5n4)c3)nc3ccccc23)cc1 ; c1ccc(-c2nc(-c3cccc(-c4ccc5ccc6cccnc6c5n4)c3)c3ccccc3n2)cc1 ; c1ccc(-c2nc(-c3cccc(-c4ccc5ccc6cccnc6c5n4)n3)nc3ccccc23)cc1]

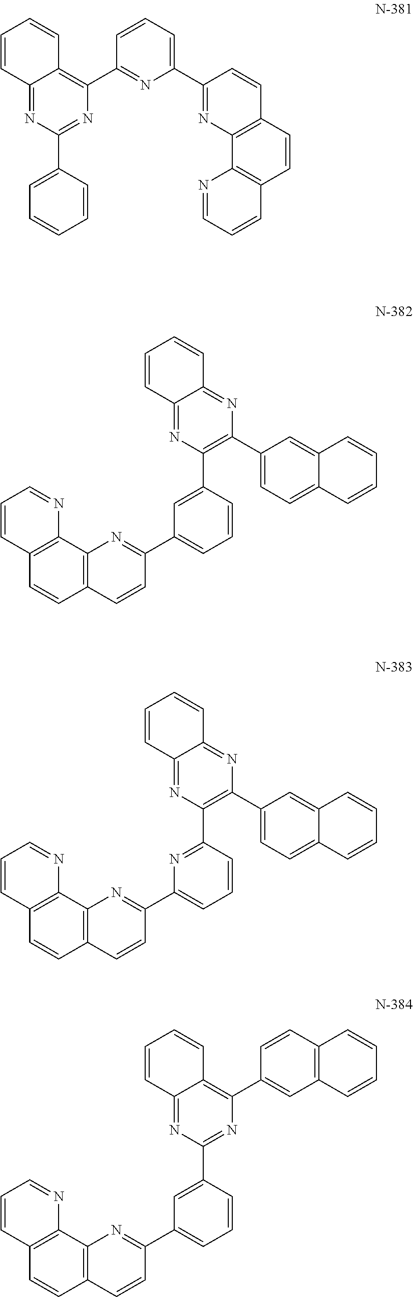 [SMILES: c1ccc(-c2nc(-c3cccc(-c4ccc5ccc6cccnc6c5n4)n3)c3ccccc3n2)cc1 ; c1cc(-c2ccc3ccc4cccnc4c3n2)cc(-c2nc3ccccc3nc2-c2ccc3ccccc3c2)c1 ; c1cc(-c2ccc3ccc4cccnc4c3n2)nc(-c2nc3ccccc3nc2-c2ccc3ccccc3c2)c1 ; c1cc(-c2ccc3ccc4cccnc4c3n2)cc(-c2nc(-c3ccc4ccccc4c3)c3ccccc3n2)c1]

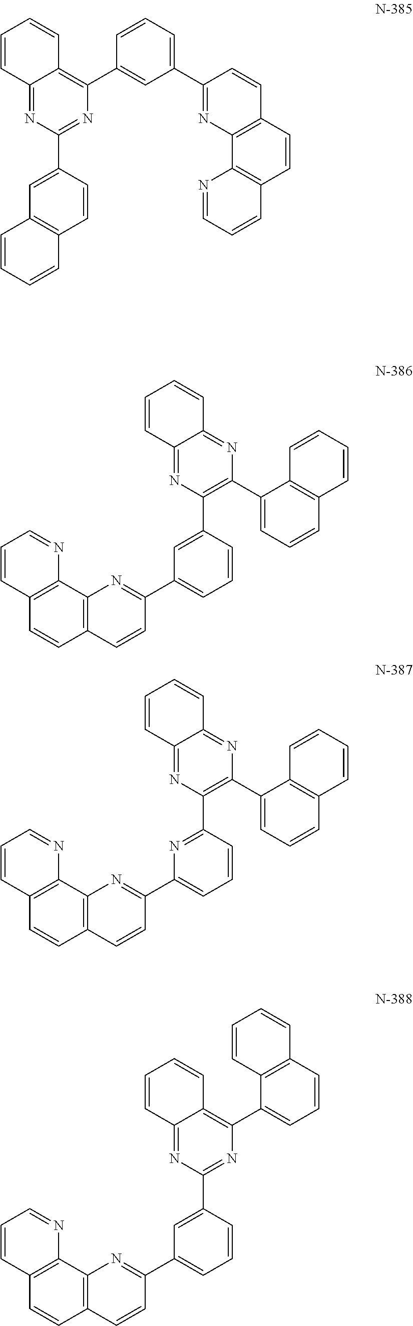 [SMILES: c1cc(-c2ccc3ccc4cccnc4c3n2)cc(-c2nc(-c3ccc4ccccc4c3)nc3ccccc23)c1 ; c1cc(-c2ccc3ccc4cccnc4c3n2)cc(-c2nc3ccccc3nc2-c2cccc3ccccc23)c1 ; c1cc(-c2ccc3ccc4cccnc4c3n2)nc(-c2nc3ccccc3nc2-c2cccc3ccccc23)c1 ; c1cc(-c2ccc3ccc4cccnc4c3n2)cc(-c2nc(-c3cccc4ccccc34)c3ccccc3n2)c1]

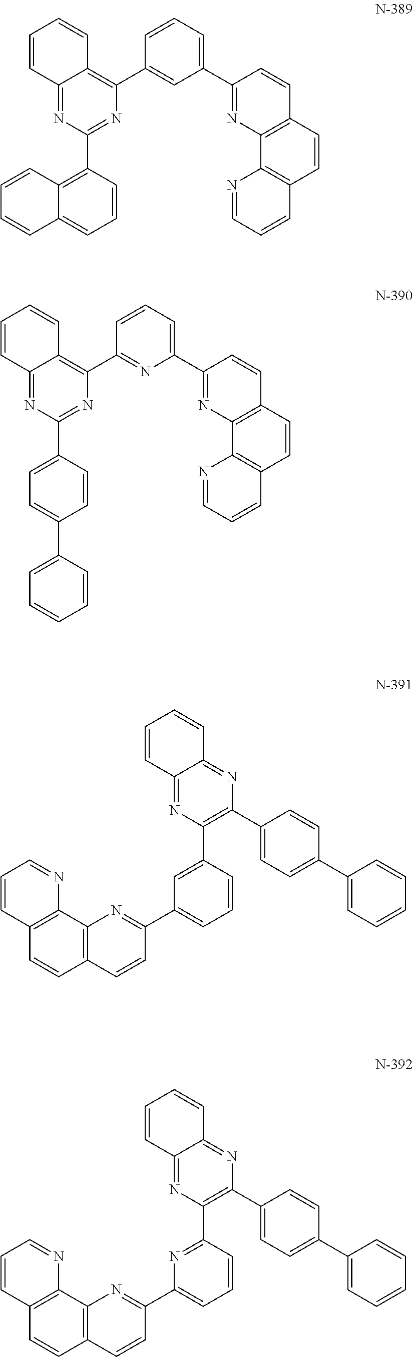 [SMILES: c1cc(-c2ccc3ccc4cccnc4c3n2)cc(-c2nc(-c3cccc4ccccc34)nc3ccccc23)c1 ; c1ccc(-c2ccc(-c3nc(-c4cccc(-c5ccc6ccc7cccnc7c6n5)n4)c4ccccc4n3)cc2)cc1 ; c1ccc(-c2ccc(-c3nc4ccccc4nc3-c3cccc(-c4ccc5ccc6cccnc6c5n4)c3)cc2)cc1 ; c1ccc(-c2ccc(-c3nc4ccccc4nc3-c3cccc(-c4ccc5ccc6cccnc6c5n4)n3)cc2)cc1]

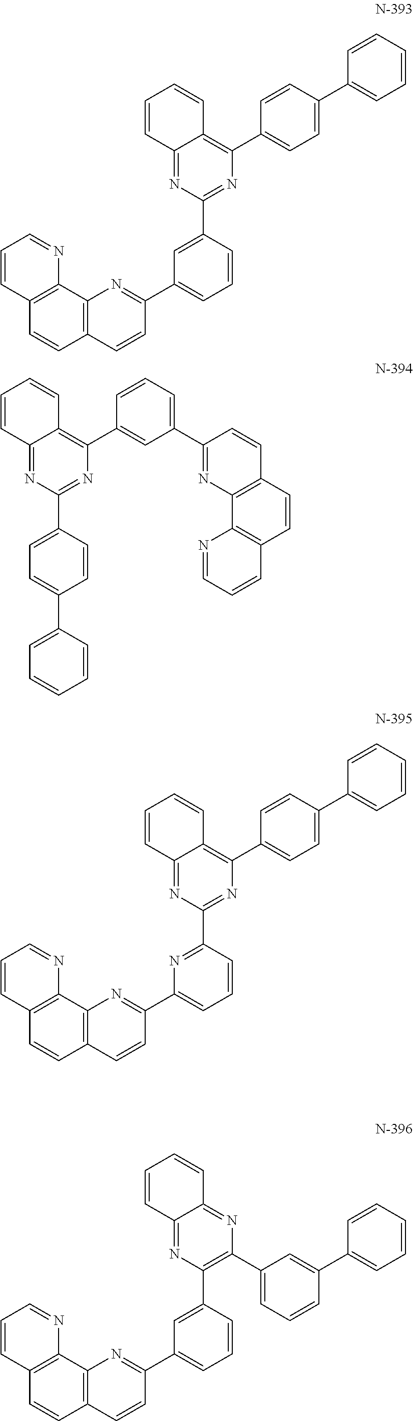

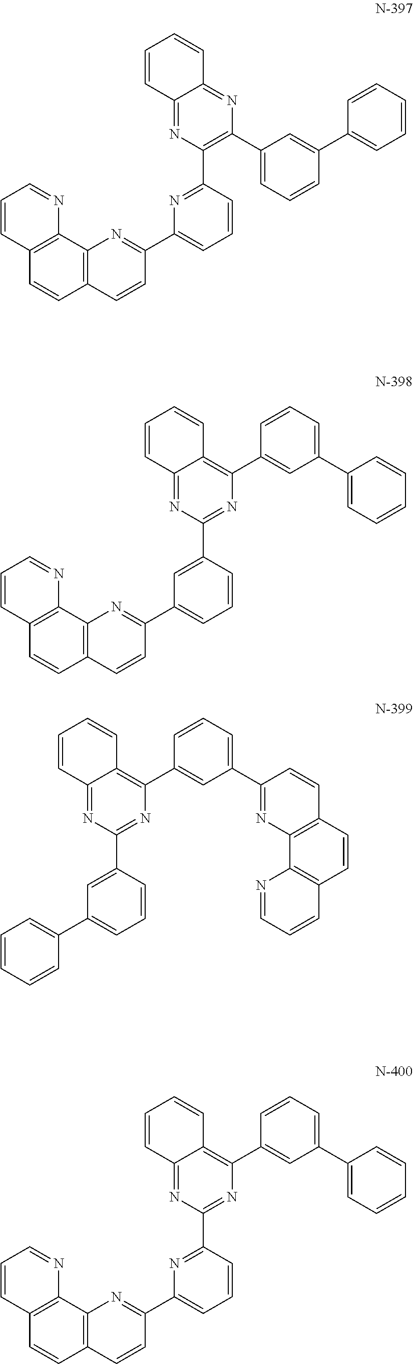 [SMILES: c1ccc(-c2cccc(-c3nc4ccccc4nc3-c3cccc(-c4ccc5ccc6cccnc6c5n4)n3)c2)cc1 ; c1ccc(-c2cccc(-c3nc(-c4cccc(-c5ccc6ccc7cccnc7c6n5)c4)nc4ccccc34)c2)cc1 ; c1ccc(-c2cccc(-c3nc(-c4cccc(-c5ccc6ccc7cccnc7c6n5)c4)c4ccccc4n3)c2)cc1 ; c1ccc(-c2cccc(-c3nc(-c4cccc(-c5ccc6ccc7cccnc7c6n5)n4)nc4ccccc34)c2)cc1]

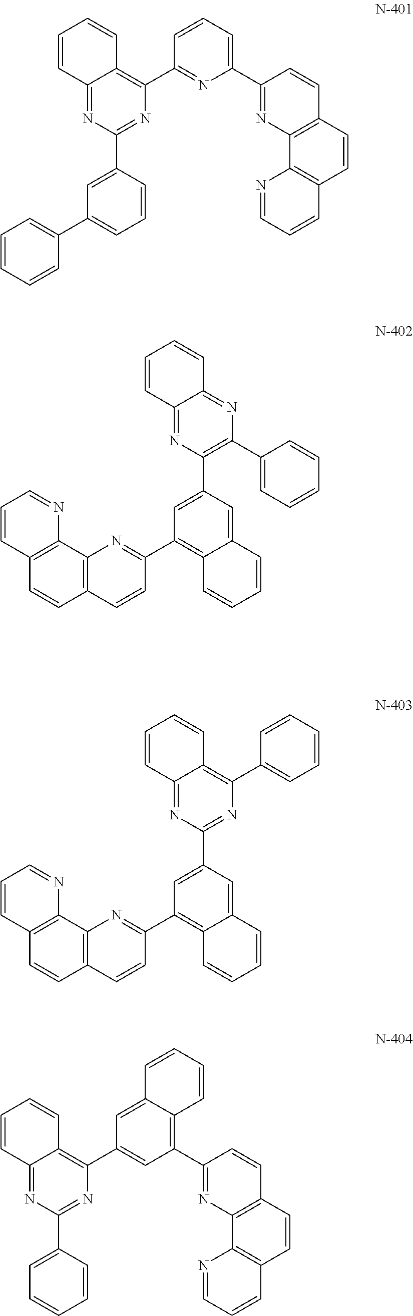 [SMILES: c1ccc(-c2cccc(-c3nc(-c4cccc(-c5ccc6ccc7cccnc7c6n5)n4)c4ccccc4n3)c2)cc1 ; c1ccc(-c2nc3ccccc3nc2-c2cc(-c3ccc4ccc5cccnc5c4n3)c3ccccc3c2)cc1 ; c1ccc(-c2nc(-c3cc(-c4ccc5ccc6cccnc6c5n4)c4ccccc4c3)nc3ccccc23)cc1 ; c1ccc(-c2nc(-c3cc(-c4ccc5ccc6cccnc6c5n4)c4ccccc4c3)c3ccccc3n2)cc1]

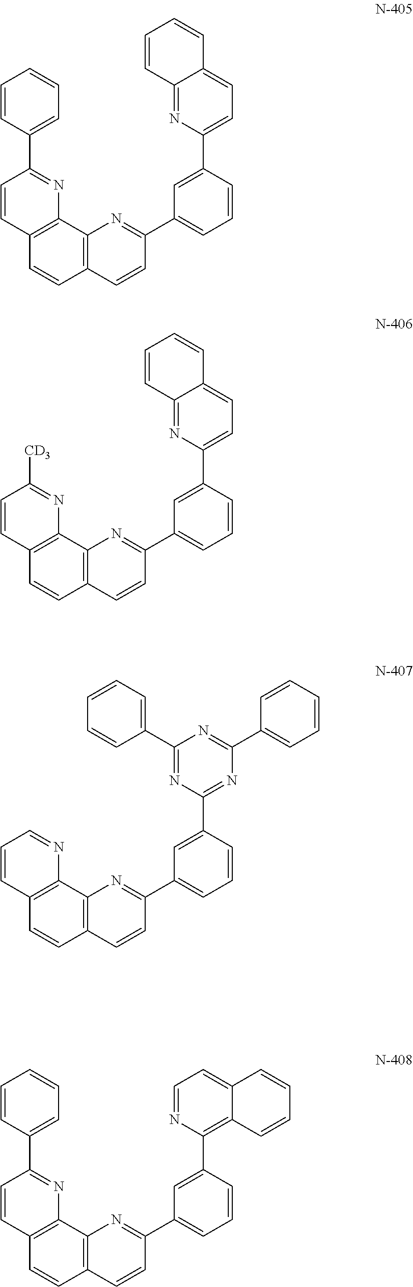 [SMILES: c1ccc(-c2ccc3ccc4ccc(-c5cccc(-c6ccc7ccccc7n6)c5)nc4c3n2)cc1 ; [2H]C([2H])([2H])c1ccc2ccc3ccc(-c4cccc(-c5ccc6ccccc6n5)c4)nc3c2n1 ; c1ccc(-c2nc(-c3ccccc3)nc(-c3cccc(-c4ccc5ccc6cccnc6c5n4)c3)n2)cc1 ; c1ccc(-c2ccc3ccc4ccc(-c5cccc(-c6nccc7ccccc67)c5)nc4c3n2)cc1]

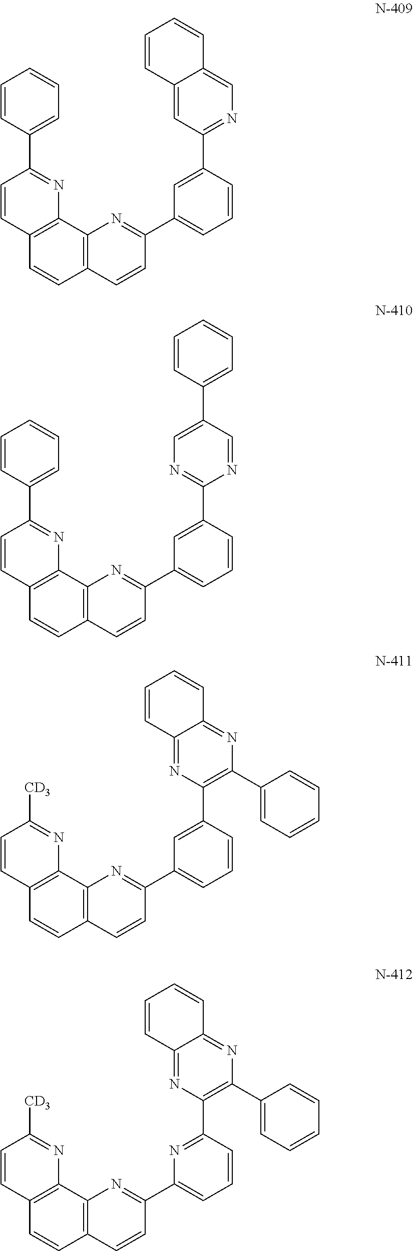 [SMILES: c1ccc(-c2ccc3ccc4ccc(-c5cccc(-c6cc7ccccc7cn6)c5)nc4c3n2)cc1 ; c1ccc(-c2cnc(-c3cccc(-c4ccc5ccc6ccc(-c7ccccc7)nc6c5n4)c3)nc2)cc1 ; [2H]C([2H])([2H])c1ccc2ccc3ccc(-c4cccc(-c5nc6ccccc6nc5-c5ccccc5)c4)nc3c2n1 ; [2H]C([2H])([2H])c1ccc2ccc3ccc(-c4cccc(-c5nc6ccccc6nc5-c5ccccc5)n4)nc3c2n1]

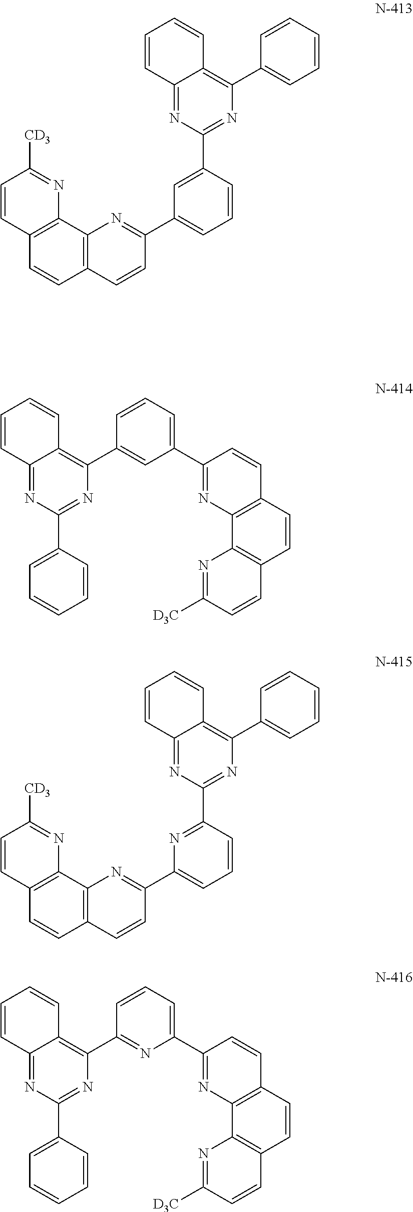 [SMILES: [2H]C([2H])([2H])c1ccc2ccc3ccc(-c4cccc(-c5nc(-c6ccccc6)c6ccccc6n5)c4)nc3c2n1 ; [2H]C([2H])([2H])c1ccc2ccc3ccc(-c4cccc(-c5nc(-c6ccccc6)nc6ccccc56)c4)nc3c2n1 ; [2H]C([2H])([2H])c1ccc2ccc3ccc(-c4cccc(-c5nc(-c6ccccc6)c6ccccc6n5)n4)nc3c2n1 ; [2H]C([2H])([2H])c1ccc2ccc3ccc(-c4cccc(-c5nc(-c6ccccc6)nc6ccccc56)n4)nc3c2n1]

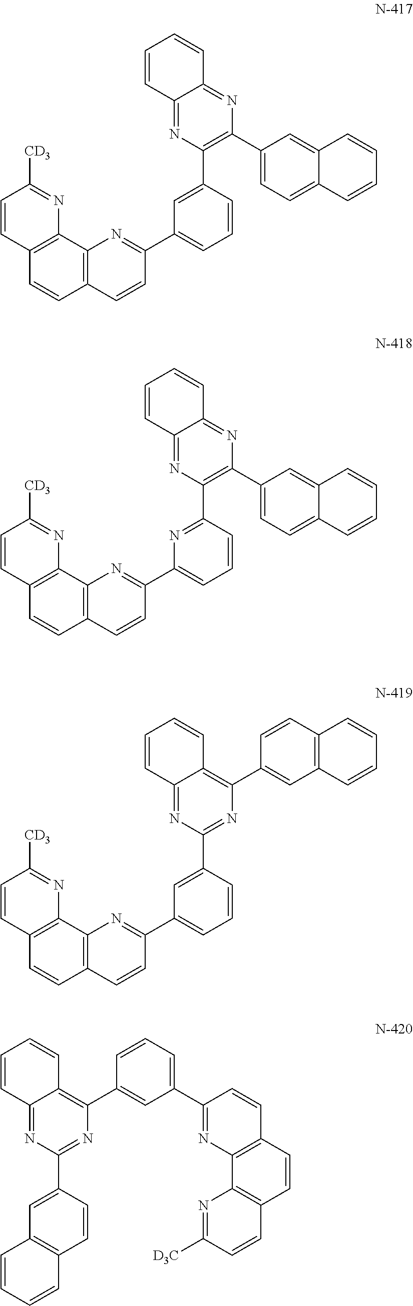 [SMILES: [2H]C([2H])([2H])c1ccc2ccc3ccc(-c4cccc(-c5nc6ccccc6nc5-c5ccc6ccccc6c5)c4)nc3c2n1 ; [2H]C([2H])([2H])c1ccc2ccc3ccc(-c4cccc(-c5nc6ccccc6nc5-c5ccc6ccccc6c5)n4)nc3c2n1 ; [2H]C([2H])([2H])c1ccc2ccc3ccc(-c4cccc(-c5nc(-c6ccc7ccccc7c6)c6ccccc6n5)c4)nc3c2n1 ; [2H]C([2H])([2H])c1ccc2ccc3ccc(-c4cccc(-c5nc(-c6ccc7ccccc7c6)nc6ccccc56)c4)nc3c2n1]

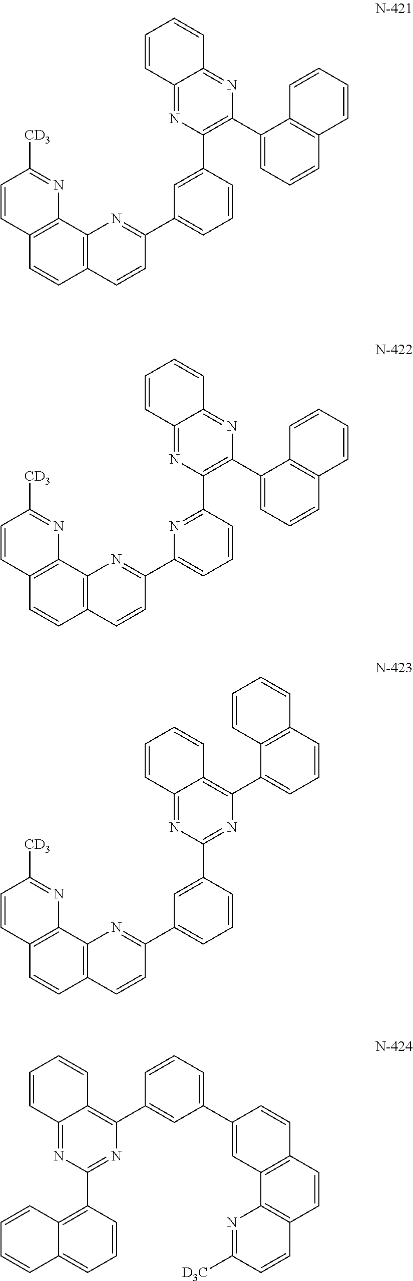 [SMILES: [2H]C([2H])([2H])c1ccc2ccc3ccc(-c4cccc(-c5nc6ccccc6nc5-c5cccc6ccccc56)c4)nc3c2n1 ; [2H]C([2H])([2H])c1ccc2ccc3ccc(-c4cccc(-c5nc6ccccc6nc5-c5cccc6ccccc56)n4)nc3c2n1 ; [2H]C([2H])([2H])c1ccc2ccc3ccc(-c4cccc(-c5nc(-c6cccc7ccccc67)c6ccccc6n5)c4)nc3c2n1 ; [2H]C([2H])([2H])c1ccc2ccc3ccc(-c4cccc(-c5nc(-c6cccc7ccccc67)nc6ccccc56)c4)cc3c2n1]

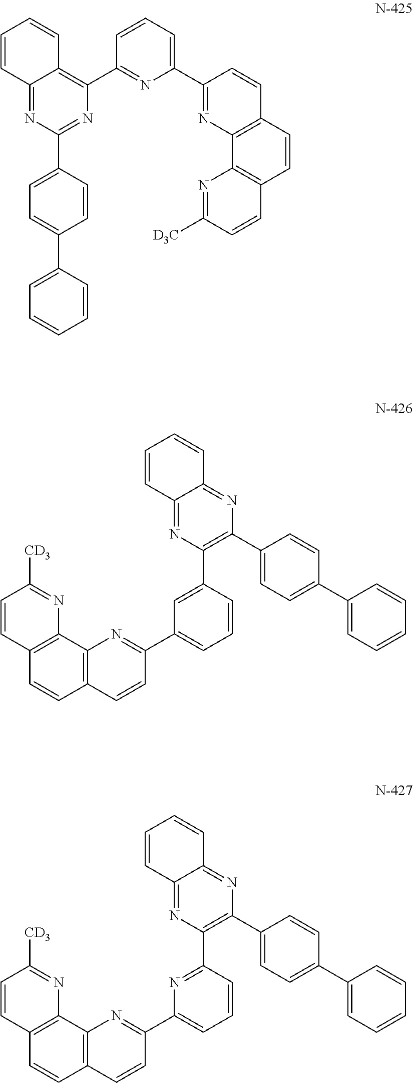 [SMILES: [2H]C([2H])([2H])c1ccc2ccc3ccc(-c4cccc(-c5nc(-c6ccc(-c7ccccc7)cc6)nc6ccccc56)n4)nc3c2n1 ; [2H]C([2H])([2H])c1ccc2ccc3ccc(-c4cccc(-c5nc6ccccc6nc5-c5ccc(-c6ccccc6)cc5)c4)nc3c2n1 ; [2H]C([2H])([2H])c1ccc2ccc3ccc(-c4cccc(-c5nc6ccccc6nc5-c5ccc(-c6ccccc6)cc5)n4)nc3c2n1]

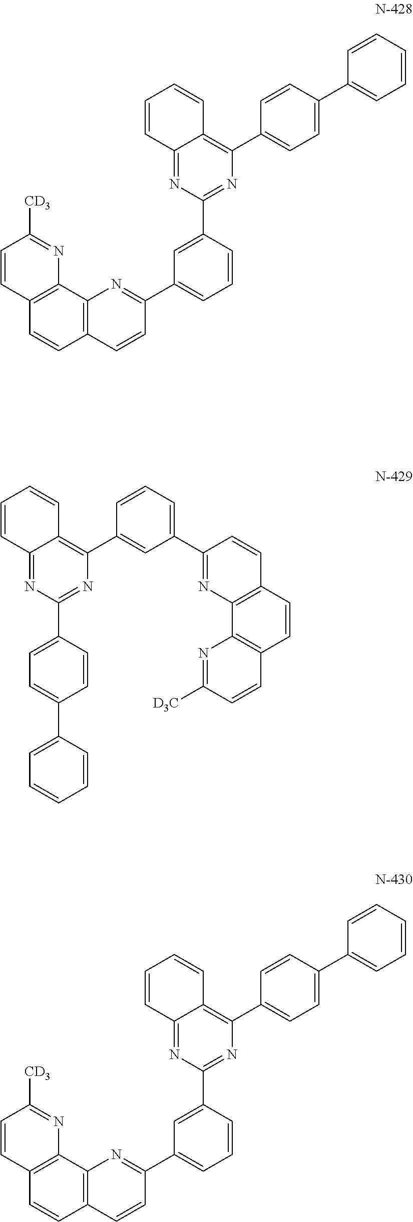 [SMILES: [2H]C([2H])([2H])c1ccc2ccc3ccc(-c4cccc(-c5nc(-c6ccc(-c7ccccc7)cc6)c6ccccc6n5)c4)nc3c2n1 ; [2H]C([2H])([2H])c1ccc2ccc3ccc(-c4cccc(-c5nc(-c6ccc(-c7ccccc7)cc6)nc6ccccc56)c4)nc3c2n1 ; [2H]C([2H])([2H])c1ccc2ccc3ccc(-c4cccc(-c5nc(-c6ccc(-c7ccccc7)cc6)c6ccccc6n5)c4)nc3c2n1]

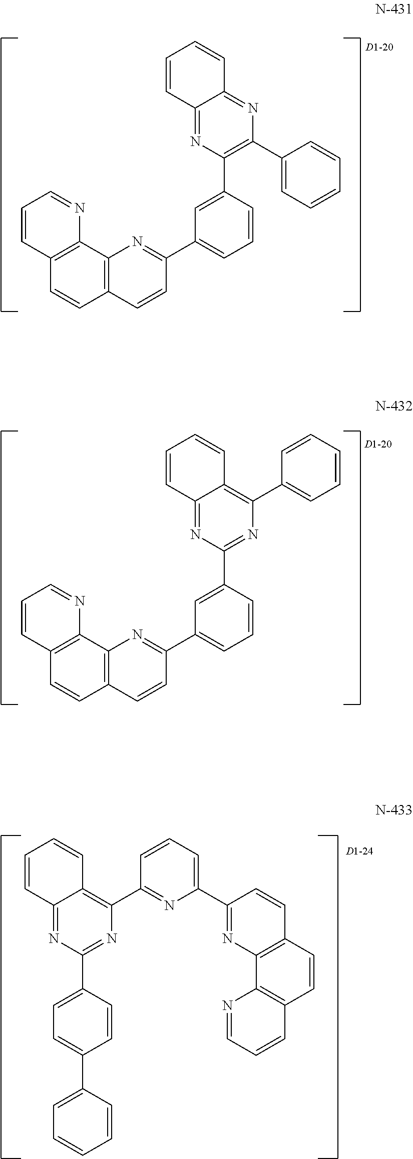

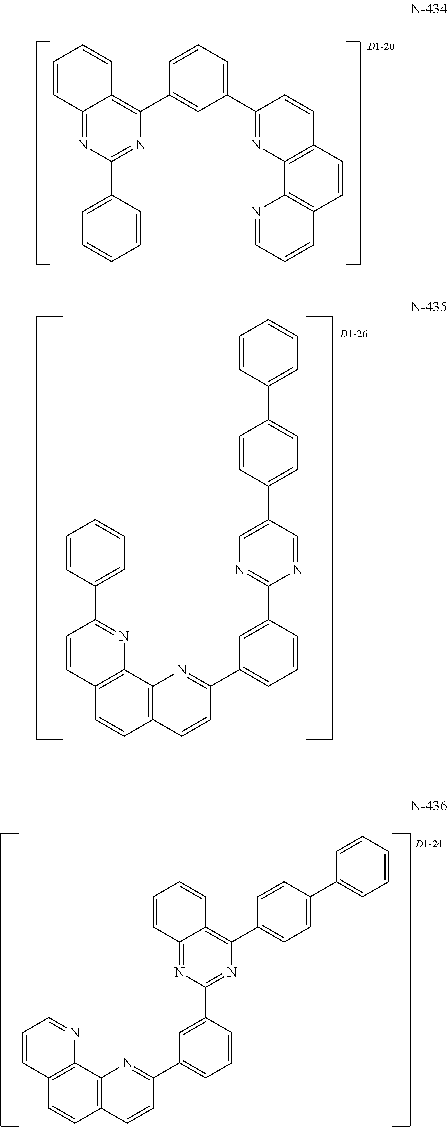

In the compounds dove, Dn means that n number of hydrogens is replaced with deuterium, wherein n represents an integer of 1 or more, wherein the upper limit of n is determined by the number of hydrogens that can be substituted in each compound.

According to another embodiment, the present disclosure provides an organic electroluminescent compound represented by the following Formula 2-1.

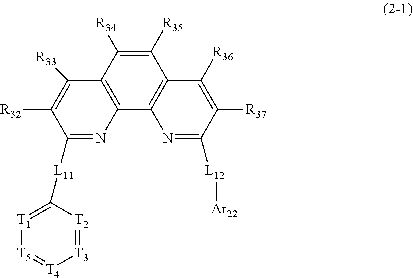

In Formula 2-1,
R₃₂ to R₃₇ each independently represent, hydrogen, deuterium, a substituted or unsubstituted (C1-C30)alkyl, a substituted or unsubstituted (C3-C30)cycloalkyl, a substituted or unsubstituted (3- to 7-membered)heterocycloalkyl, or a substituted or unsubstituted (C6-C30)aryl; or may be linked to the adjacent substituents to form a ring(s);
L₁₁ represents a single bond, a substituted or unsubstituted (3- to 30-membered)heteroarylene, a substituted or unsubstituted naphthylene, a substituted or unsubstituted o-phenylene, a substituted or unsubstituted m-phenylene, or a substituted or unsubstituted p-phenylene;
L₁₂ represents a single bond, a substituted or unsubstituted (C1-C30)alkylene, a substituted or unsubstituted (C6-C30)arylene, a substituted or unsubstituted (3- to 30-membered)heteroarylene, or a substituted or unsubstituted (C3-C30)cycloalkylene;
T₁ to T₅ each independently represent, CRₐ or N; provided that at least one of T₁ to T₅ represent(s) N;
Rₐ represents hydrogen, deuterium, a substituted or unsubstituted (C1-C30)alkyl, a substituted or unsubstituted (C6-C30)aryl, or a substituted or unsubstituted (3- to 30-membered)heteroaryl; or may be linked to the adjacent substituents to form a ring(s); and
Ar₂ represents hydrogen, deuterium, a substituted or unsubstituted (C1-C6)alkyl, a substituted or unsubstituted (C6-C30)aryl, or a substituted or unsubstituted (3- to 30-membered)heteroaryl;
provided that in Formula 2-1, the compounds where L₁₁ and L₁₂ are a single bond or phenylene, Ar₂ is naphthyl, dimethylfluorenyl, phenanthrenyl, pyrenyl, triphenylenyl, or fluoranthenyl, and

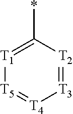

is pyridyl, pyrimidyl, quinolinyl, diphenyl-substituted triazinyl, dipyridyl-substituted pyridyl, or benzoquinolinyl, are excluded.

In one embodiment, at least one of T₁ to T₅ may be N, preferably at least two of T₁ to T₅ may be N, more preferably at least three of T₁ to T₅ may be N.

In one embodiment,

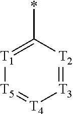

in Formula 2-1 may be represented by the following Formula 2-1-1 or 2-1-2.

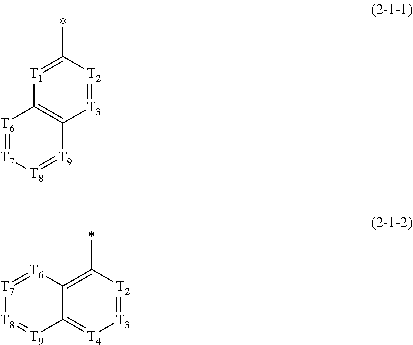

In Formulas 2-1-1 and 2-1-2,
T₁ to T₄ are as defined in Formula 2-1;
T₆ to T₉ each independently represent, CRₐ; and
Rₐ is as defined in Formula 2-1.

In one embodiment, Rₐ represents hydrogen, a substituted or unsubstituted (C6-C30)aryl, or a substituted or unsubstituted (5- to 30-membered)heteroaryl; or may be linked to the adjacent substituents to form a ring(s), preferably hydrogen, a substituted or unsubstituted (C6-C25)aryl, or a substituted or unsubstituted (5- to 25-membered)heteroaryl; or may be linked to the adjacent substituents to form a substituted or unsubstituted (5- to 30-membered) monocyclic or polycyclic aromatic ring, more preferably hydrogen, a substituted or unsubstituted (C6-C18)aryl, or a substituted or unsubstituted (5- to 18-membered)heteroaryl; or may be linked to the adjacent substituents to form a substituted or unsubstituted (5- to 25-membered) monocyclic or polycyclic aromatic ring. For example, Rₐ may be hydrogen, a substituted or unsubstituted phenyl, naphthyl unsubstituted or substituted with phenyl, a substituted or unsubstituted m-biphenyl, a substituted or unsubstituted o-biphenyl, a substituted or unsubstituted phenanthrenyl, a substituted or unsubstituted pyridyl, quinazolyl unsubstituted or substituted with phenyl, naphthyl, or biphenyl, quinoxalinyl unsubstituted or substituted with phenyl, naphthyl, or biphenyl, a substituted or unsubstituted dimethylfluorenyl, a substituted or unsubstituted diphenylfluorenyl, a substituted or unsubstituted dibenzothiophenyl, or a substituted or unsubstituted dibenzofuranyl; or may be linked to the adjacent substituents to form a benzene ring(s).

In one embodiment, L₁₂ may be a single bond, a substituted or unsubstituted (C6-C30)arylene, or a substituted or unsubstituted (5- to 30-membered)heteroarylene, preferably a single bond, a substituted or unsubstituted (C6-C25)arylene, or a substituted or unsubstituted (5- to 25-membered)heteroarylene, more preferably a single bond, a substituted or unsubstituted (C6-C18)arylene, or a substituted or unsubstituted (5- to 18-membered)heteroarylene. For example, L₁₂ may be a single bond or a substituted or unsubstituted phenylene.

In one embodiment, Ar₂₂ may be hydrogen, deuterium, a substituted or unsubstituted (C1-C6)alkyl, a substituted or unsubstituted (C6-C30)aryl or a substituted or unsubstituted (5- to 30-membered)heteroaryl, preferably hydrogen, a substituted or unsubstituted (C1-C6)alkyl, a substituted or unsubstituted (C6-C25)aryl, or a substituted or unsubstituted (5- to 25-membered)heteroaryl, more preferably hydrogen, (C1-C6)alkyl unsubstituted or substituted with deuterium, a substituted or unsubstituted (C6-C18)aryl, or a substituted or unsubstituted (5- to 18-membered)heteroaryl. For example, Ar₂₂ may be hydrogen, CD₃, tert-butyl, a substituted or unsubstituted phenyl, a substituted or unsubstituted o-biphenyl, a substituted or unsubstituted m-biphenyl, a substituted or unsubstituted p-biphenyl, a substituted or unsubstituted o-terphenyl, a substituted or unsubstituted dimethylfluorenyl, a substituted or unsubstituted diethylfluorenyl, a substituted or unsubstituted diphenylfluorenyl, a substituted or unsubstituted anthracenyl, a substituted or unsubstituted phenanthrenyl, a substituted or unsubstituted pyrenyl, a substituted or unsubstituted triphenylenyl, a substituted or unsubstituted dibenzofuranyl, or a substituted or unsubstituted dibenzothiophenyl.

In one embodiment, R₃₂ to R₃₇ each independently may be, hydrogen, a substituted or unsubstituted (C1-C30)alkyl, or a substituted or unsubstituted (C6-C30)aryl; or may be linked to the adjacent substituents to form a ring(s), preferably hydrogen, a substituted or unsubstituted (C1-C10)alkyl, or a substituted or unsubstituted (C6-C25)aryl; or may be linked to the adjacent substituents to form a substituted or unsubstituted (5- to 30-membered) monocyclic or polycyclic aromatic ring, more preferably hydrogen, a substituted or unsubstituted (C1-C10)alkyl, or a substituted or unsubstituted (C6-C18)aryl; or may be linked to the adjacent substituents to form a substituted or unsubstituted (5- to 25-membered) monocyclic or polycyclic aromatic ring. For example, R₃₂ to R₃₇ each independently may be, hydrogen, methyl, phenyl, m-biphenyl; or R₃₄ and R₃₅ may be linked to form a benzene ring, or R₃₆ and R₃₇ may be linked to form a benzene ring.

According to one embodiment, the compound represented by Formula 2-1 may be more specifically illustrated by the following compounds, but is not limited thereto.

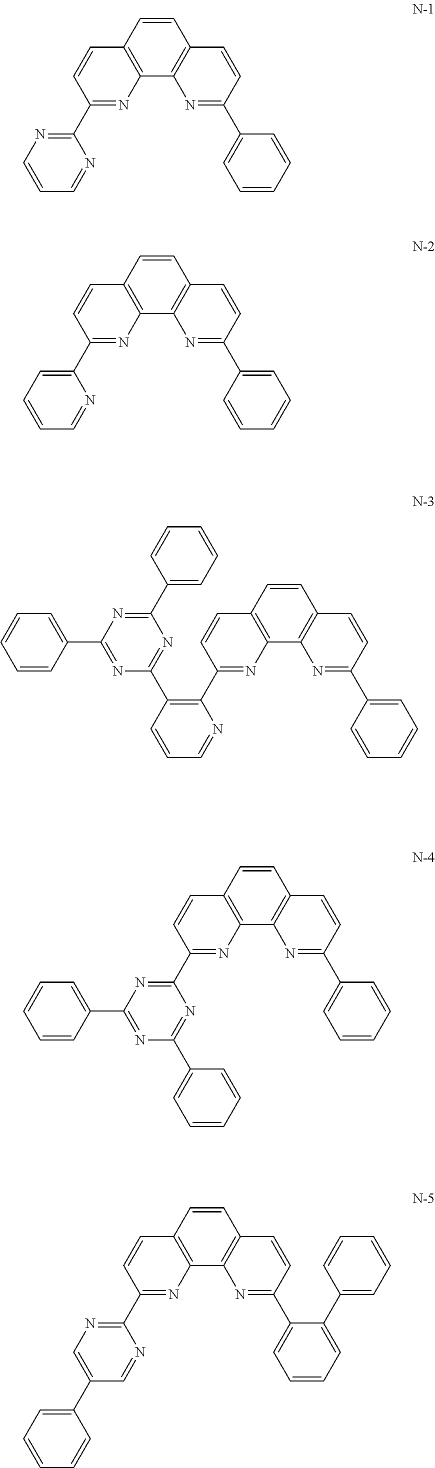 [SMILES: c1ccc(-c2ccc3ccc4ccc(-c5ncccn5)nc4c3n2)cc1 ; c1ccc(-c2ccc3ccc4ccc(-c5ccccn5)nc4c3n2)cc1 ; c1ccc(-c2ccc3ccc4ccc(-c5ncccc5-c5nc(-c6ccccc6)nc(-c6ccccc6)n5)nc4c3n2)cc1 ; c1ccc(-c2ccc3ccc4ccc(-c5nc(-c6ccccc6)nc(-c6ccccc6)n5)nc4c3n2)cc1 ; c1ccc(-c2cnc(-c3ccc4ccc5ccc(-c6ccccc6-c6ccccc6)nc5c4n3)nc2)cc1]

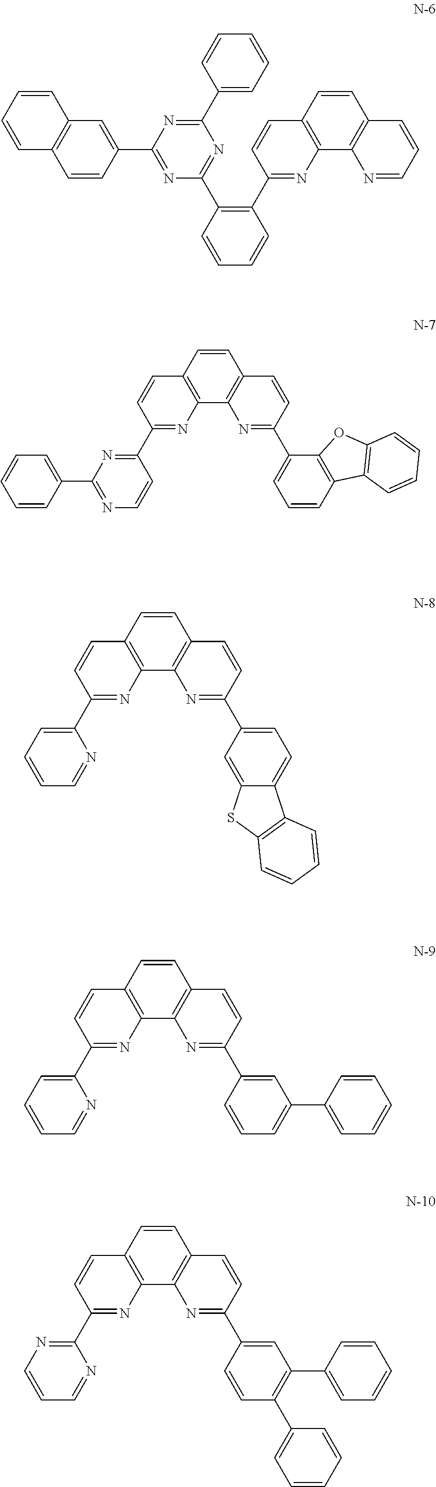 [SMILES: c1ccc(-c2nc(-c3ccc4ccccc4c3)nc(-c3ccccc3-c3ccc4ccc5cccnc5c4n3)n2)cc1 ; c1ccc(-c2nccc(-c3ccc4ccc5ccc(-c6cccc7c6oc6ccccc67)nc5c4n3)n2)cc1 ; c1ccc(-c2ccc3ccc4ccc(-c5ccc6c(c5)sc5ccccc56)nc4c3n2)nc1 ; c1ccc(-c2cccc(-c3ccc4ccc5ccc(-c6ccccn6)nc5c4n3)c2)cc1 ; c1ccc(-c2ccc(-c3ccc4ccc5ccc(-c6ncccn6)nc5c4n3)cc2-c2ccccc2)cc1]

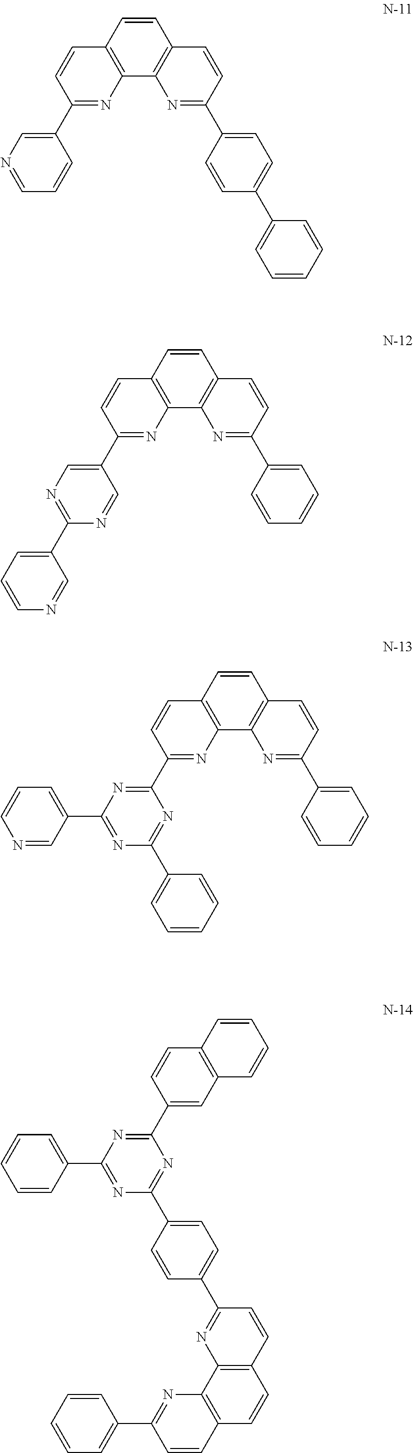 [SMILES: c1ccc(-c2ccc(-c3ccc4ccc5ccc(-c6cccnc6)nc5c4n3)cc2)cc1 ; c1ccc(-c2ccc3ccc4ccc(-c5cnc(-c6cccnc6)nc5)nc4c3n2)cc1 ; c1ccc(-c2ccc3ccc4ccc(-c5nc(-c6ccccc6)nc(-c6cccnc6)n5)nc4c3n2)cc1 ; c1ccc(-c2ccc3ccc4ccc(-c5ccc(-c6nc(-c7ccccc7)nc(-c7ccc8ccccc8c7)n6)cc5)nc4c3n2)cc1]

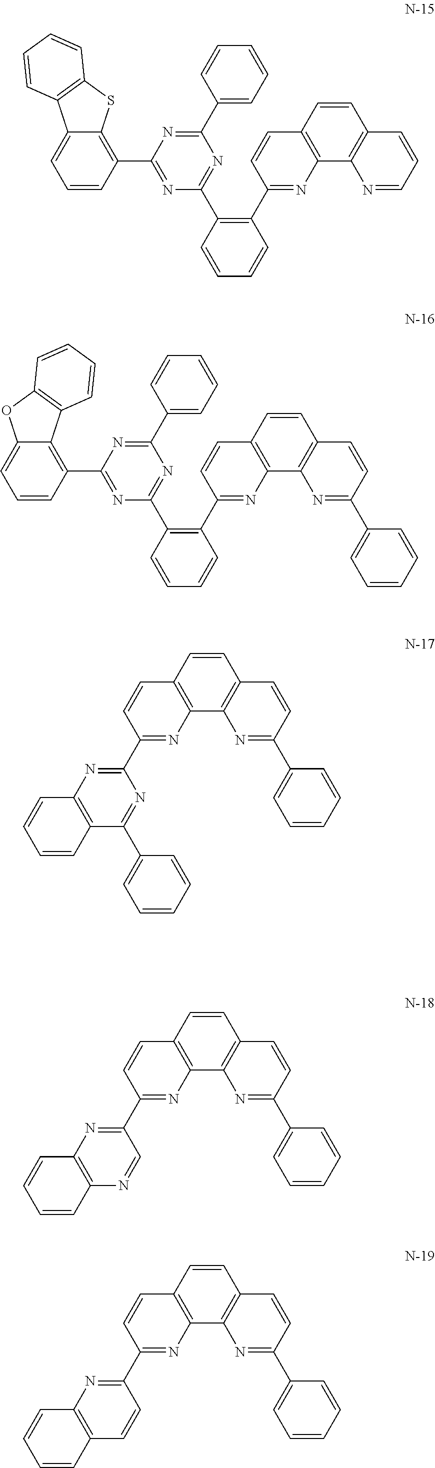 [SMILES: c1ccc(-c2nc(-c3ccccc3-c3ccc4ccc5cccnc5c4n3)nc(-c3cccc4c3sc3ccccc34)n2)cc1 ; c1ccc(-c2ccc3ccc4ccc(-c5ccccc5-c5nc(-c6ccccc6)nc(-c6cccc7oc8ccccc8c67)n5)nc4c3n2)cc1 ; c1ccc(-c2ccc3ccc4ccc(-c5nc(-c6ccccc6)c6ccccc6n5)nc4c3n2)cc1 ; c1ccc(-c2ccc3ccc4ccc(-c5cnc6ccccc6n5)nc4c3n2)cc1 ; c1ccc(-c2ccc3ccc4ccc(-c5ccc6ccccc6n5)nc4c3n2)cc1]

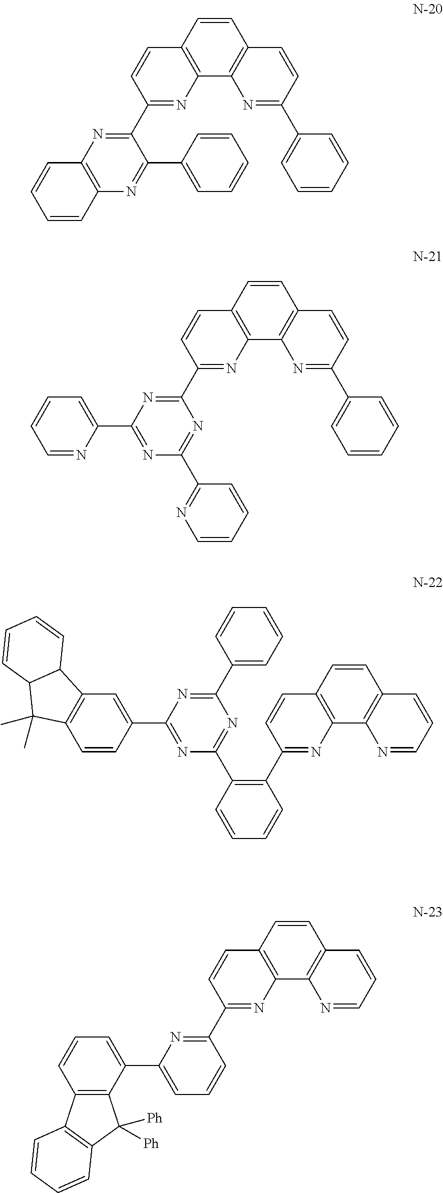 [SMILES: c1ccc(-c2ccc3ccc4ccc(-c5nc6ccccc6nc5-c5ccccc5)nc4c3n2)cc1 ; c1ccc(-c2ccc3ccc4ccc(-c5nc(-c6ccccn6)nc(-c6ccccn6)n5)nc4c3n2)cc1 ; CC1(C)c2ccc(-c3nc(-c4ccccc4)nc(-c4ccccc4-c4ccc5ccc6cccnc6c5n4)n3)cc2C2C=CC=CC21 ; c1ccc(C2(c3ccccc3)c3ccccc3-c3cccc(-c4cccc(-c5ccc6ccc7cccnc7c6n5)n4)c32)cc1]

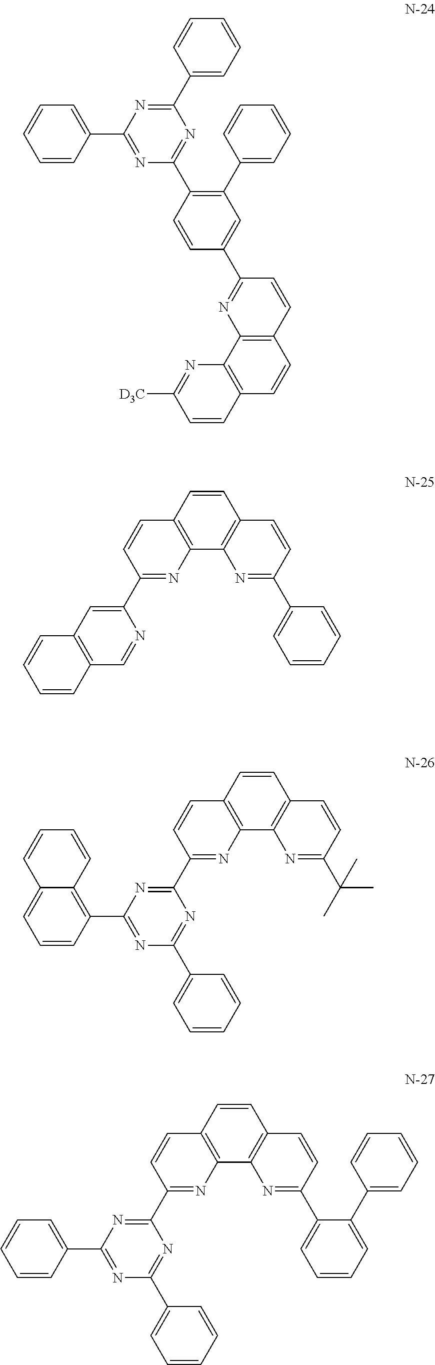 [SMILES: [2H]C([2H])([2H])c1ccc2ccc3ccc(-c4ccc(-c5nc(-c6ccccc6)nc(-c6ccccc6)n5)c(-c5ccccc5)c4)nc3c2n1 ; c1ccc(-c2ccc3ccc4ccc(-c5cc6ccccc6cn5)nc4c3n2)cc1 ; CC(C)(C)c1ccc2ccc3ccc(-c4nc(-c5ccccc5)nc(-c5cccc6ccccc56)n4)nc3c2n1 ; c1ccc(-c2nc(-c3ccccc3)nc(-c3ccc4ccc5ccc(-c6ccccc6-c6ccccc6)nc5c4n3)n2)cc1]

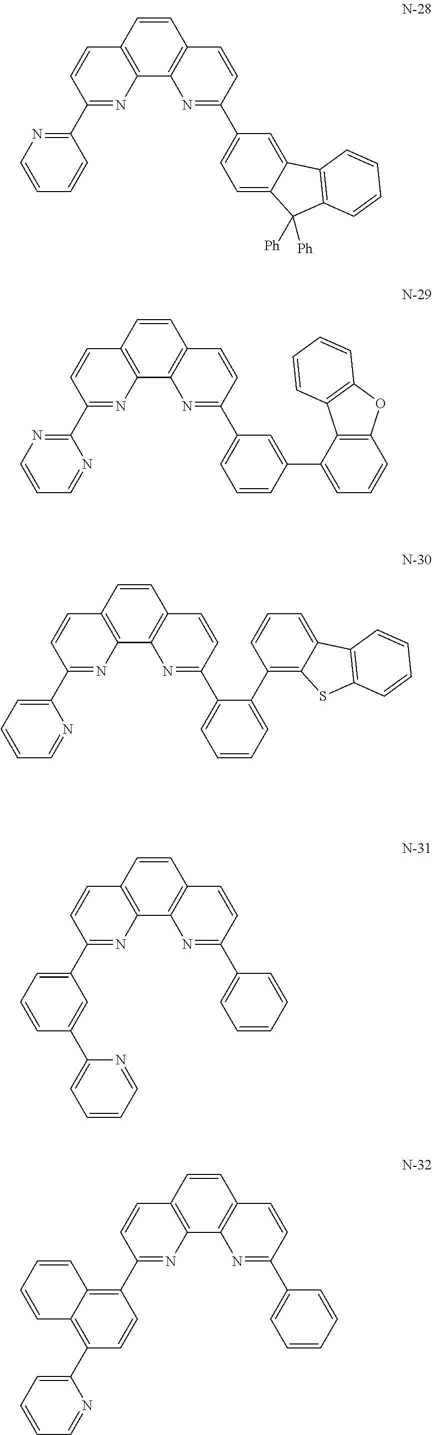 [SMILES: c1ccc(C2(c3ccccc3)c3ccccc3-c3cc(-c4ccc5ccc6ccc(-c7ccccn7)nc6c5n4)ccc32)cc1 ; c1cnc(-c2ccc3ccc4ccc(-c5cccc(-c6cccc7oc8ccccc8c67)c5)nc4c3n2)nc1 ; c1ccc(-c2ccc3ccc4ccc(-c5ccccc5-c5cccc6c5sc5ccccc56)nc4c3n2)nc1 ; c1ccc(-c2ccc3ccc4ccc(-c5cccc(-c6ccccn6)c5)nc4c3n2)cc1 ; c1ccc(-c2ccc3ccc4ccc(-c5ccc(-c6ccccn6)c6ccccc56)nc4c3n2)cc1]

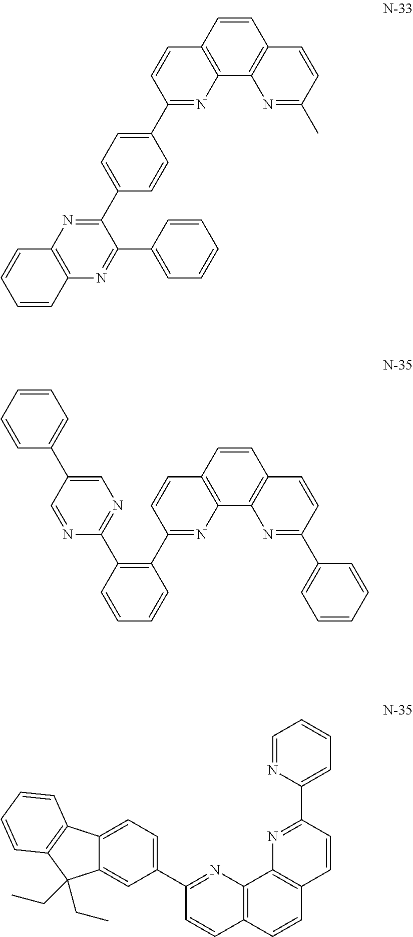 [SMILES: Cc1ccc2ccc3ccc(-c4ccc(-c5nc6ccccc6nc5-c5ccccc5)cc4)nc3c2n1 ; c1ccc(-c2cnc(-c3ccccc3-c3ccc4ccc5ccc(-c6ccccc6)nc5c4n3)nc2)cc1 ; CCC1(CC)c2ccccc2-c2ccc(-c3ccc4ccc5ccc(-c6ccccn6)nc5c4n3)cc21]

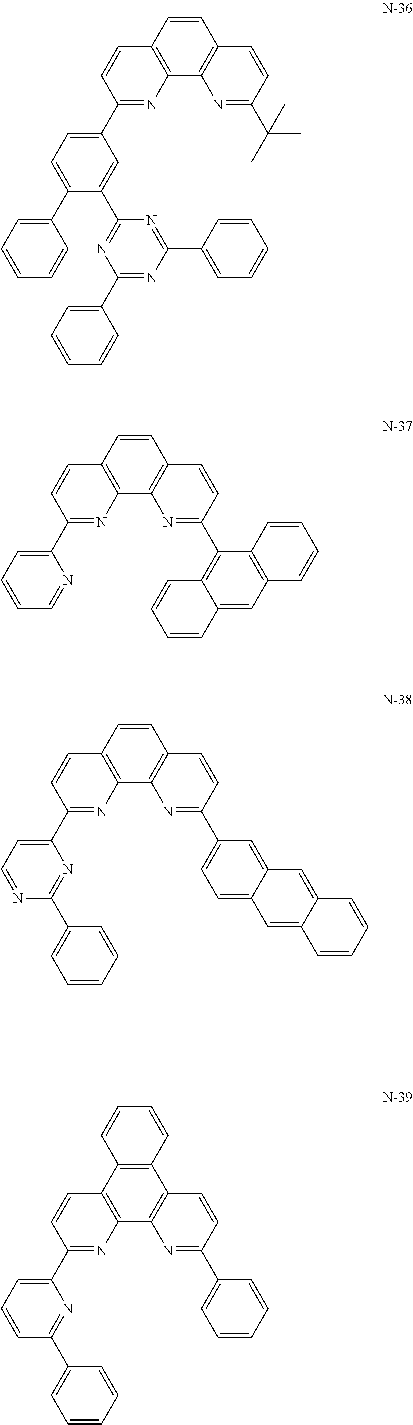 [SMILES: CC(C)(C)c1ccc2ccc3ccc(-c4ccc(-c5ccccc5)c(-c5nc(-c6ccccc6)nc(-c6ccccc6)n5)c4)nc3c2n1 ; c1ccc(-c2ccc3ccc4ccc(-c5c6ccccc6cc6ccccc56)nc4c3n2)nc1 ; c1ccc(-c2nccc(-c3ccc4ccc5ccc(-c6ccc7cc8ccccc8cc7c6)nc5c4n3)n2)cc1 ; c1ccc(-c2cccc(-c3ccc4c5ccccc5c5ccc(-c6ccccc6)nc5c4n3)n2)cc1]

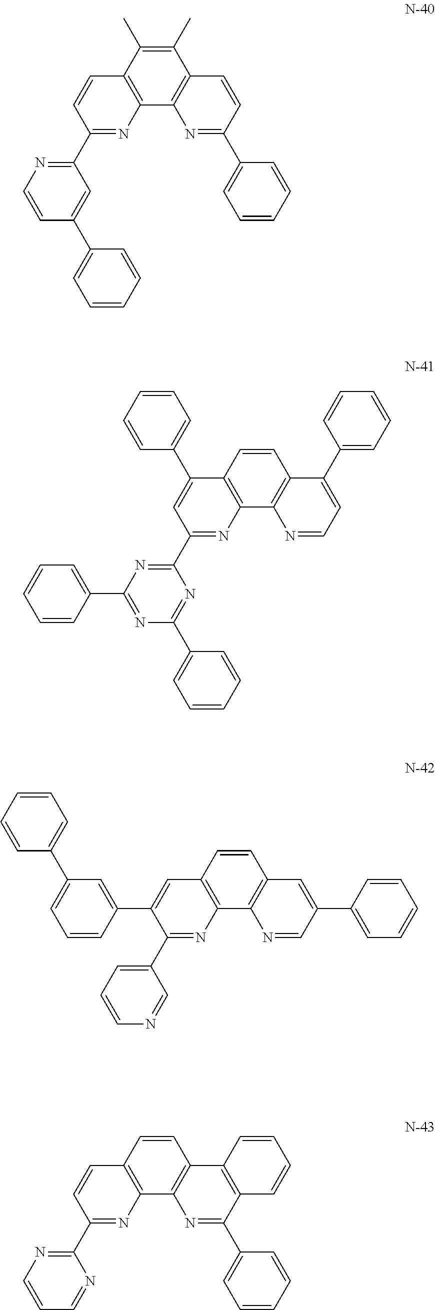 [SMILES: Cc1c(C)c2ccc(-c3cc(-c4ccccc4)ccn3)nc2c2nc(-c3ccccc3)ccc12 ; c1ccc(-c2nc(-c3ccccc3)nc(-c3cc(-c4ccccc4)c4ccc5c(-c6ccccc6)ccnc5c4n3)n2)cc1 ; c1ccc(-c2cccc(-c3cc4ccc5cc(-c6ccccc6)cnc5c4nc3-c3cccnc3)c2)cc1 ; c1ccc(-c2nc3c(ccc4ccc(-c5ncccn5)nc43)c3ccccc23)cc1]

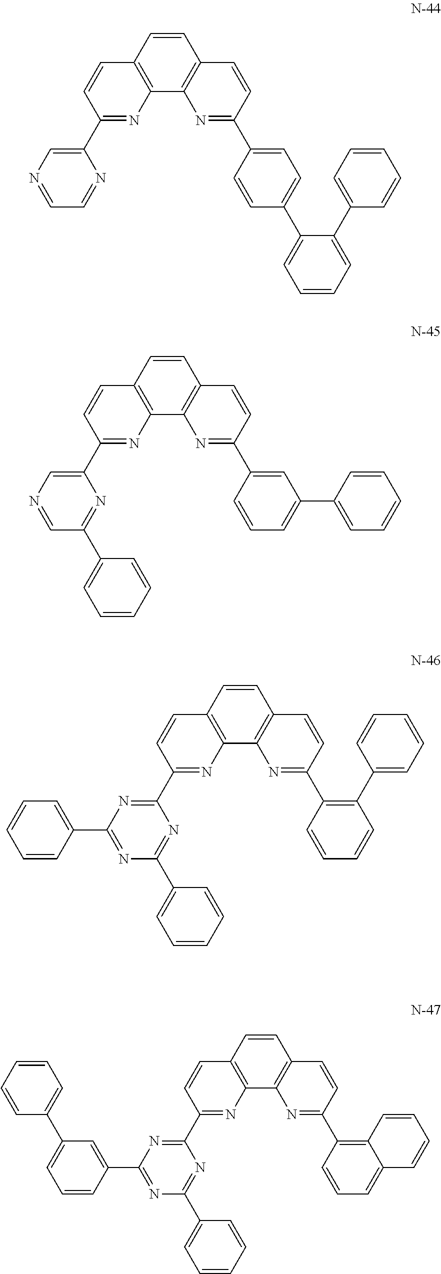 [SMILES: c1ccc(-c2ccccc2-c2ccc(-c3ccc4ccc5ccc(-c6cnccn6)nc5c4n3)cc2)cc1 ; c1ccc(-c2cccc(-c3ccc4ccc5ccc(-c6cncc(-c7ccccc7)n6)nc5c4n3)c2)cc1 ; c1ccc(-c2nc(-c3ccccc3)nc(-c3ccc4ccc5ccc(-c6ccccc6-c6ccccc6)nc5c4n3)n2)cc1 ; c1ccc(-c2cccc(-c3nc(-c4ccccc4)nc(-c4ccc5ccc6ccc(-c7cccc8ccccc78)nc6c5n4)n3)c2)cc1]

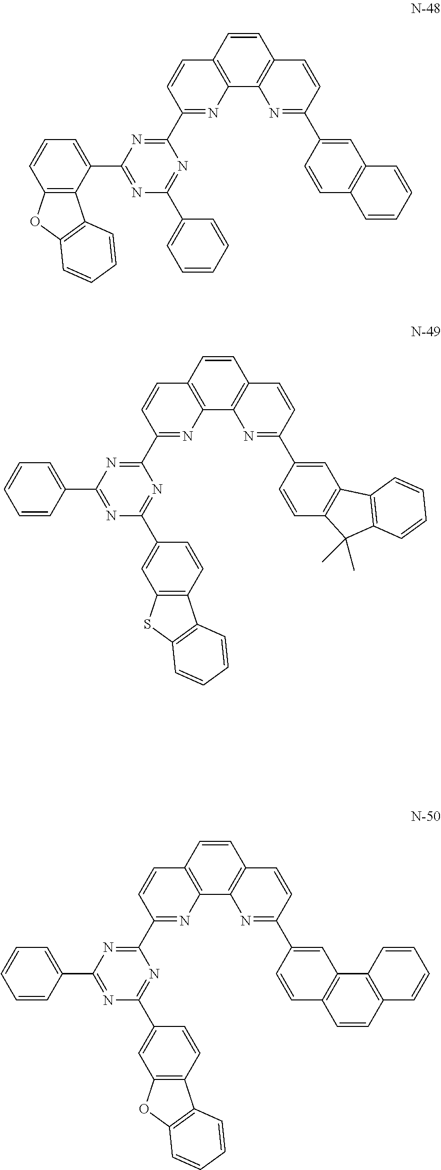 [SMILES: c1ccc(-c2nc(-c3ccc4ccc5ccc(-c6ccc7ccccc7c6)nc5c4n3)nc(-c3cccc4oc5ccccc5c34)n2)cc1 ; CC1(C)c2ccccc2-c2cc(-c3ccc4ccc5ccc(-c6nc(-c7ccccc7)nc(-c7ccc8c(c7)sc7ccccc78)n6)nc5c4n3)ccc21 ; c1ccc(-c2nc(-c3ccc4c(c3)oc3ccccc34)nc(-c3ccc4ccc5ccc(-c6ccc7ccc8ccccc8c7c6)nc5c4n3)n2)cc1]

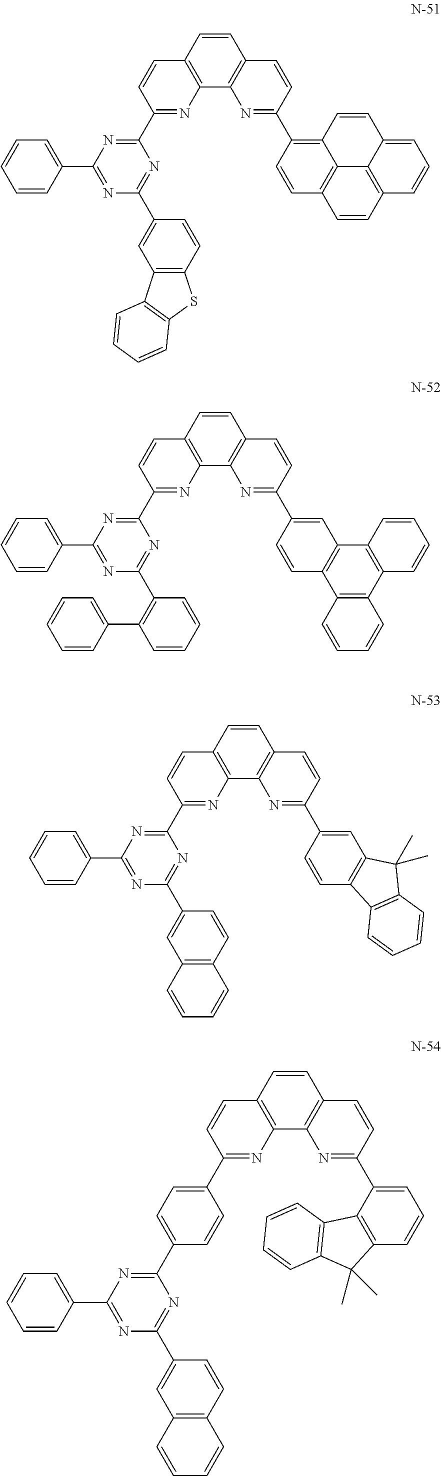 [SMILES: c1ccc(-c2nc(-c3ccc4sc5ccccc5c4c3)nc(-c3ccc4ccc5ccc(-c6ccc7ccc8cccc9ccc6c7c89)nc5c4n3)n2)cc1 ; c1ccc(-c2nc(-c3ccc4ccc5ccc(-c6ccc7c8ccccc8c8ccccc8c7c6)nc5c4n3)nc(-c3ccccc3-c3ccccc3)n2)cc1 ; CC1(C)c2ccccc2-c2ccc(-c3ccc4ccc5ccc(-c6nc(-c7ccccc7)nc(-c7ccc8ccccc8c7)n6)nc5c4n3)cc21 ; CC1(C)c2ccccc2-c2c(-c3ccc4ccc5ccc(-c6ccc(-c7nc(-c8ccccc8)nc(-c8ccc9ccccc9c8)n7)cc6)nc5c4n3)cccc21]

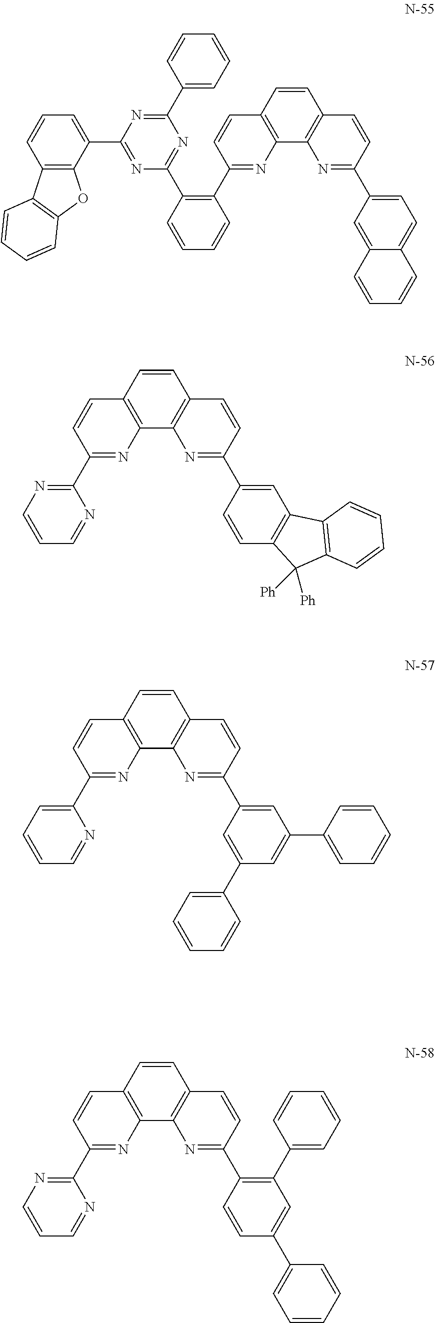 [SMILES: c1ccc(-c2nc(-c3ccccc3-c3ccc4ccc5ccc(-c6ccc7ccccc7c6)nc5c4n3)nc(-c3cccc4c3oc3ccccc34)n2)cc1 ; c1ccc(C2(c3ccccc3)c3ccccc3-c3cc(-c4ccc5ccc6ccc(-c7ncccn7)nc6c5n4)ccc32)cc1 ; c1ccc(-c2cc(-c3ccccc3)cc(-c3ccc4ccc5ccc(-c6ccccn6)nc5c4n3)c2)cc1 ; c1ccc(-c2ccc(-c3ccc4ccc5ccc(-c6ncccn6)nc5c4n3)c(-c3ccccc3)c2)cc1]

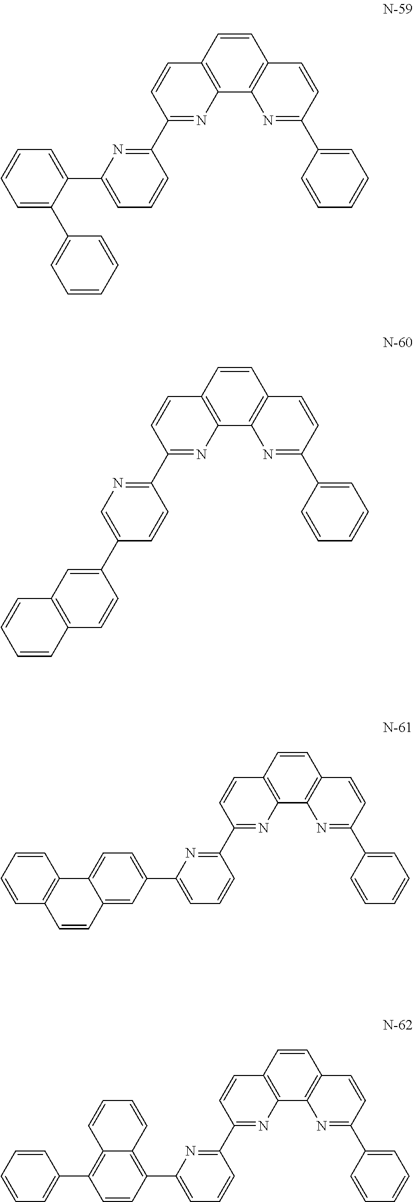 [SMILES: c1ccc(-c2ccc3ccc4ccc(-c5cccc(-c6ccccc6-c6ccccc6)n5)nc4c3n2)cc1 ; c1ccc(-c2ccc3ccc4ccc(-c5ccc(-c6ccc7ccccc7c6)cn5)nc4c3n2)cc1 ; c1ccc(-c2ccc3ccc4ccc(-c5cccc(-c6ccc7c(ccc8ccccc87)c6)n5)nc4c3n2)cc1 ; c1ccc(-c2ccc3ccc4ccc(-c5cccc(-c6ccc(-c7ccccc7)c7ccccc67)n5)nc4c3n2)cc1]

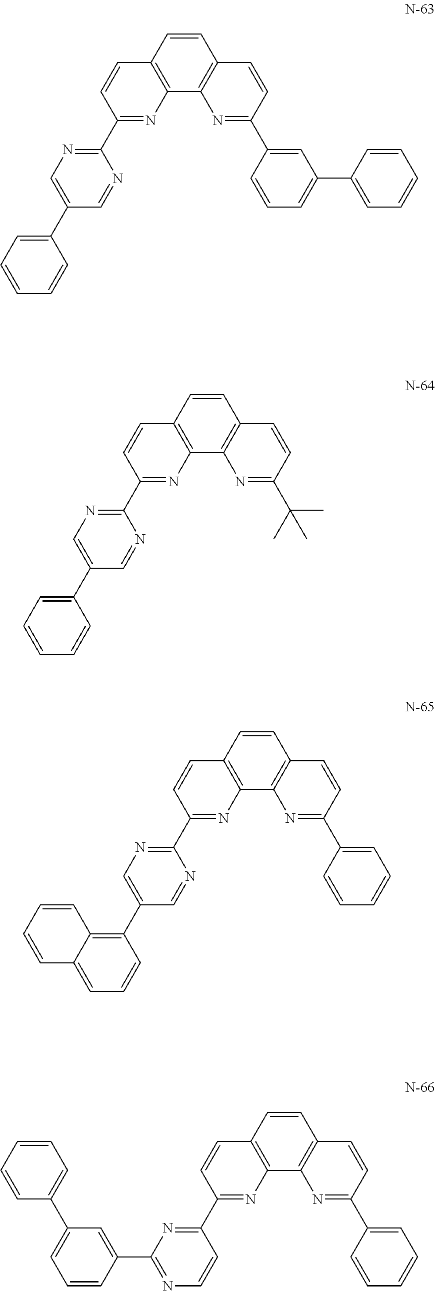 [SMILES: c1ccc(-c2cnc(-c3ccc4ccc5ccc(-c6cccc(-c7ccccc7)c6)nc5c4n3)nc2)cc1 ; CC(C)(C)c1ccc2ccc3ccc(-c4ncc(-c5ccccc5)cn4)nc3c2n1 ; c1ccc(-c2ccc3ccc4ccc(-c5ncc(-c6cccc7ccccc67)cn5)nc4c3n2)cc1 ; c1ccc(-c2cccc(-c3nccc(-c4ccc5ccc6ccc(-c7ccccc7)nc6c5n4)n3)c2)cc1]

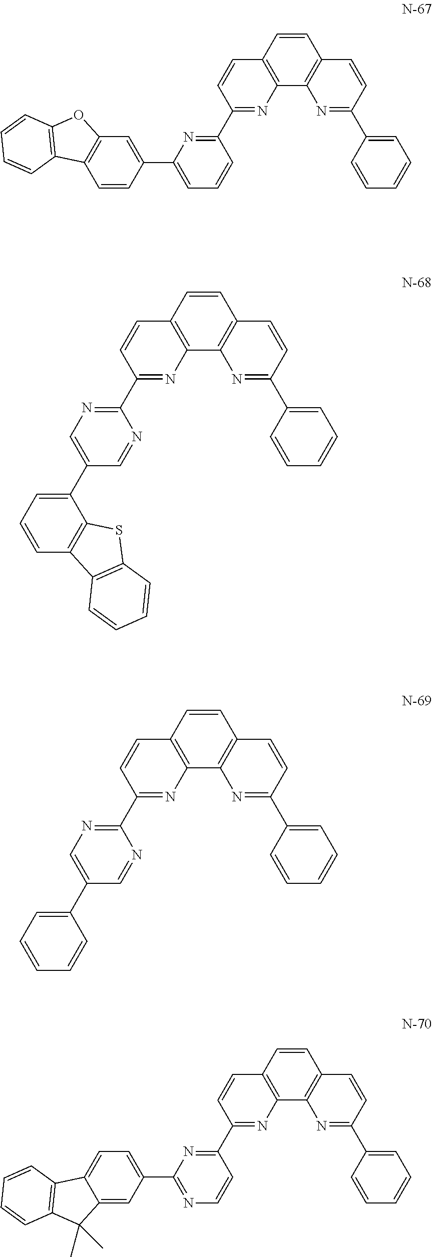 [SMILES: c1ccc(-c2ccc3ccc4ccc(-c5cccc(-c6ccc7c(c6)oc6ccccc67)n5)nc4c3n2)cc1 ; c1ccc(-c2ccc3ccc4ccc(-c5ncc(-c6cccc7c6sc6ccccc67)cn5)nc4c3n2)cc1 ; c1ccc(-c2cnc(-c3ccc4ccc5ccc(-c6ccccc6)nc5c4n3)nc2)cc1 ; CC1(C)c2ccccc2-c2ccc(-c3nccc(-c4ccc5ccc6ccc(-c7ccccc7)nc6c5n4)n3)cc21]

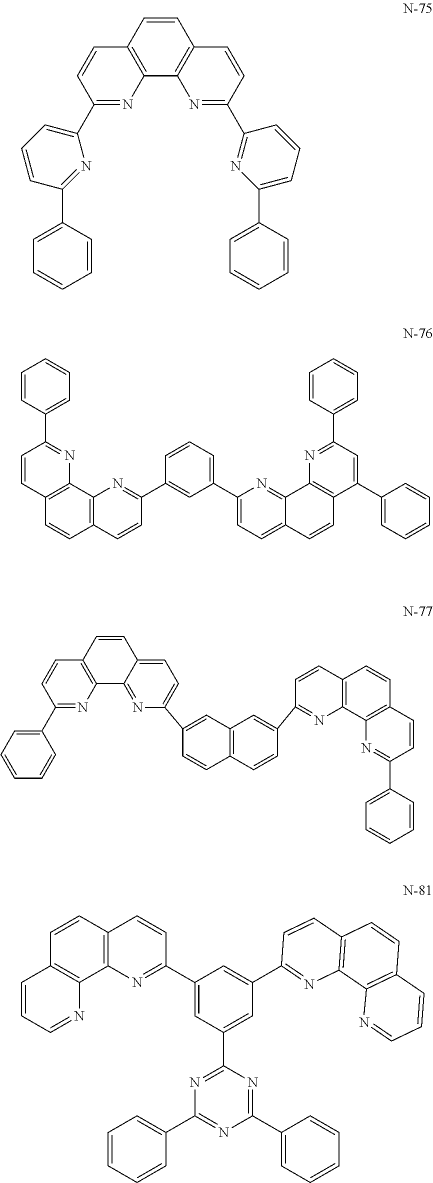 [SMILES: c1ccc(-c2cccc(-c3ccc4ccc5ccc(-c6cccc(-c7ccccc7)n6)nc5c4n3)n2)cc1 ; c1ccc(-c2ccc3ccc4ccc(-c5cccc(-c6ccc7ccc8c(-c9ccccc9)cc(-c9ccccc9)nc8c7n6)c5)nc4c3n2)cc1 ; c1ccc(-c2ccc3ccc4ccc(-c5ccc6ccc(-c7ccc8ccc9ccc(-c%10ccccc%10)nc9c8n7)cc6c5)nc4c3n2)cc1 ; c1ccc(-c2nc(-c3ccccc3)nc(-c3cc(-c4ccc5ccc6cccnc6c5n4)cc(-c4ccc5ccc6cccnc6c5n4)c3)n2)cc1]

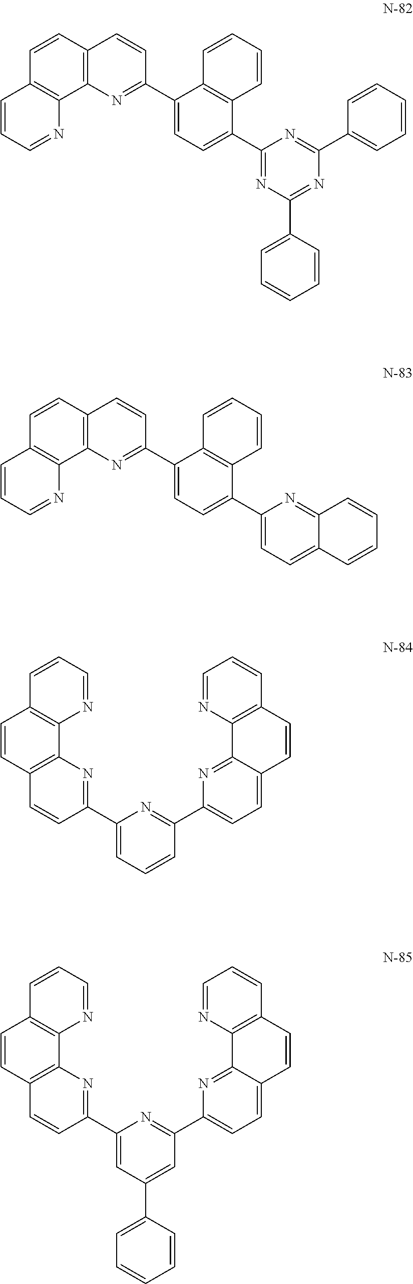 [SMILES: c1ccc(-c2nc(-c3ccccc3)nc(-c3ccc(-c4ccc5ccc6cccnc6c5n4)c4ccccc34)n2)cc1 ; c1ccc2nc(-c3ccc(-c4ccc5ccc6cccnc6c5n4)c4ccccc34)ccc2c1 ; c1cc(-c2ccc3ccc4cccnc4c3n2)nc(-c2ccc3ccc4cccnc4c3n2)c1 ; c1ccc(-c2cc(-c3ccc4ccc5cccnc5c4n3)nc(-c3ccc4ccc5cccnc5c4n3)c2)cc1]

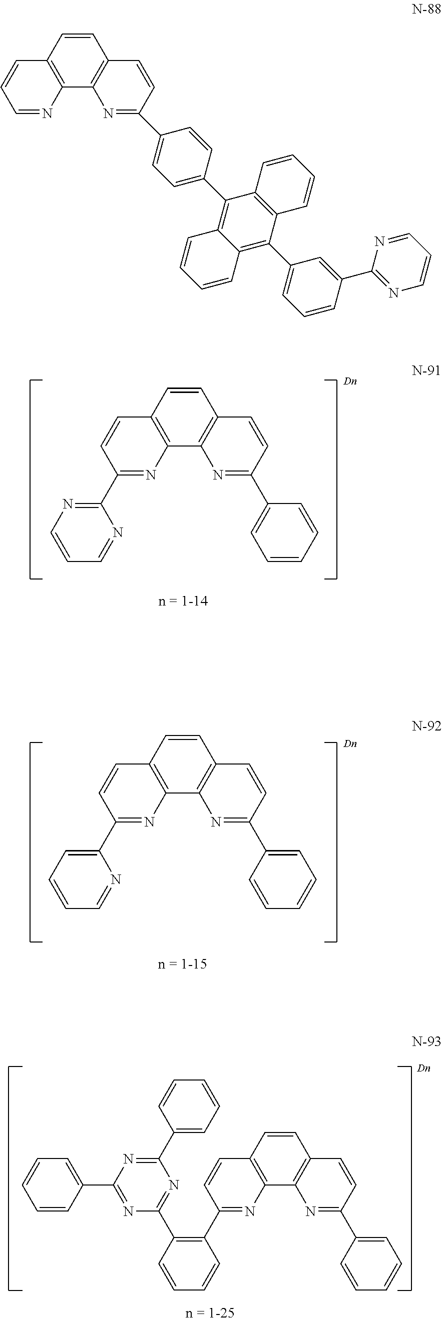

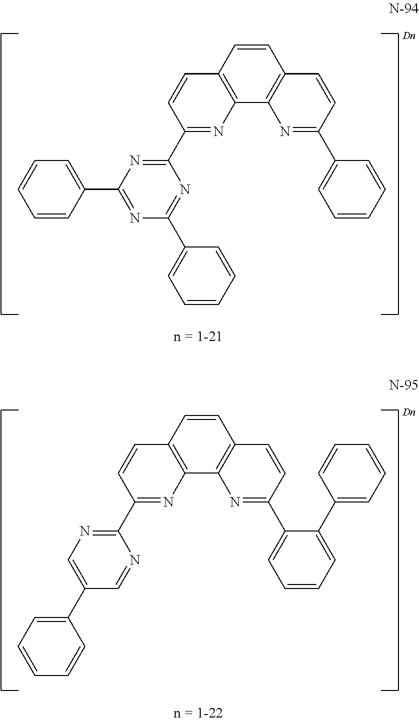

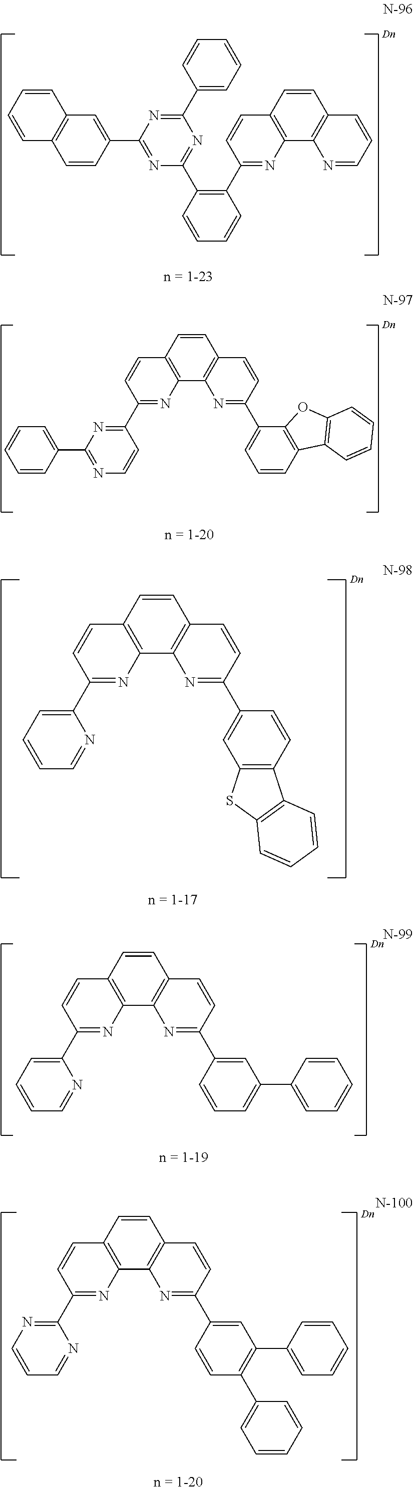

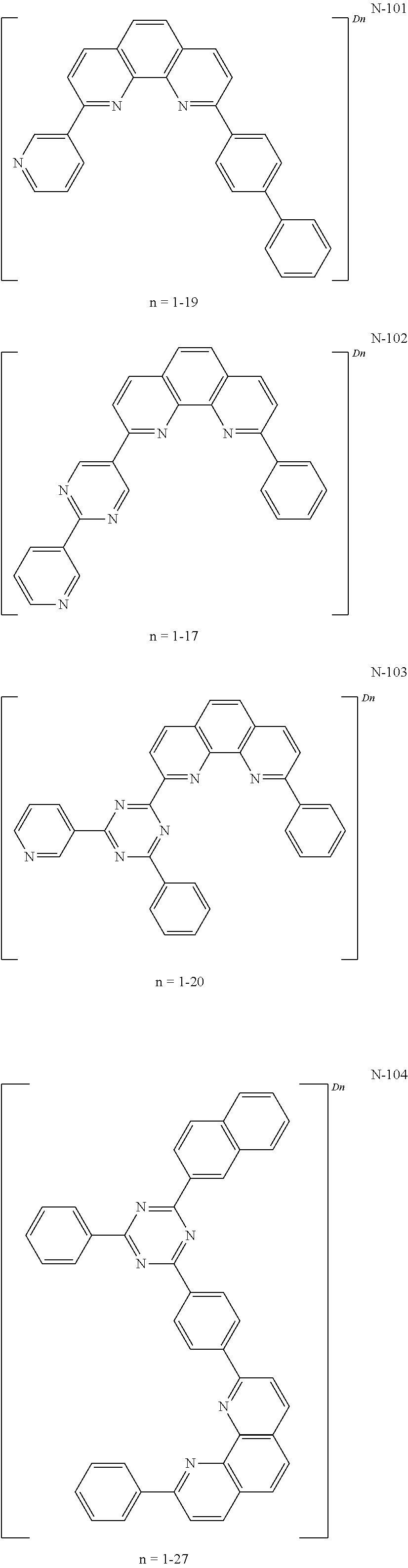

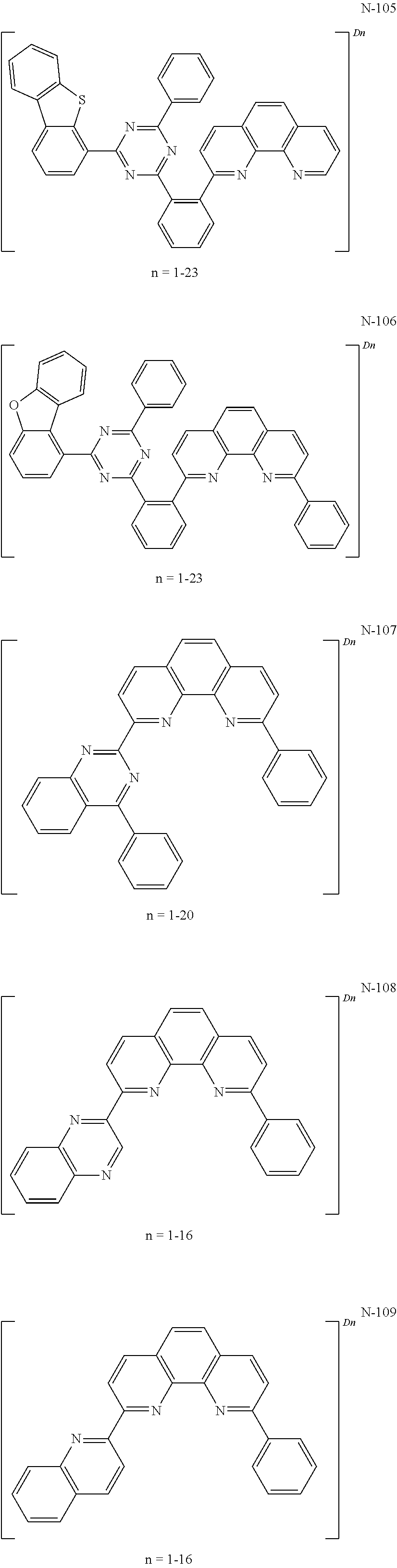

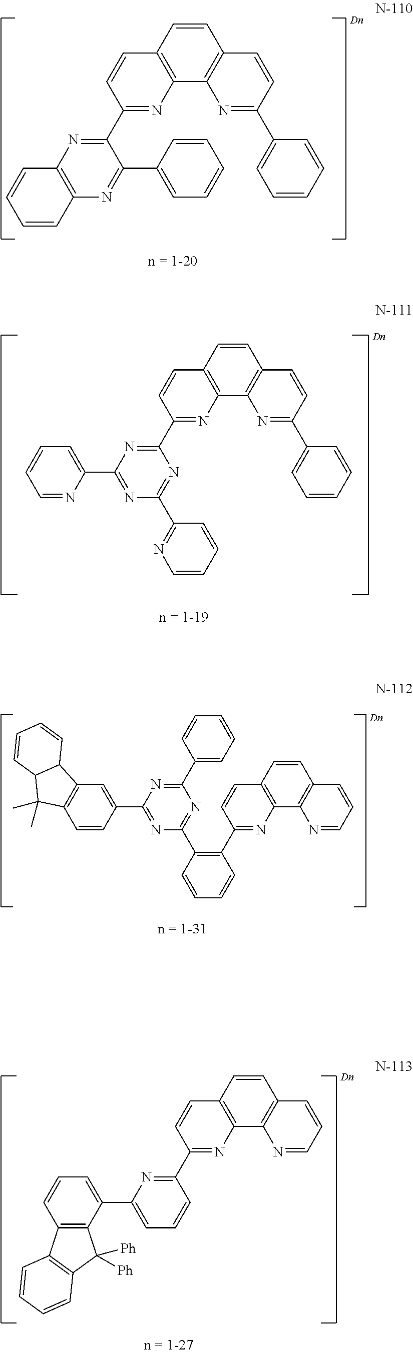

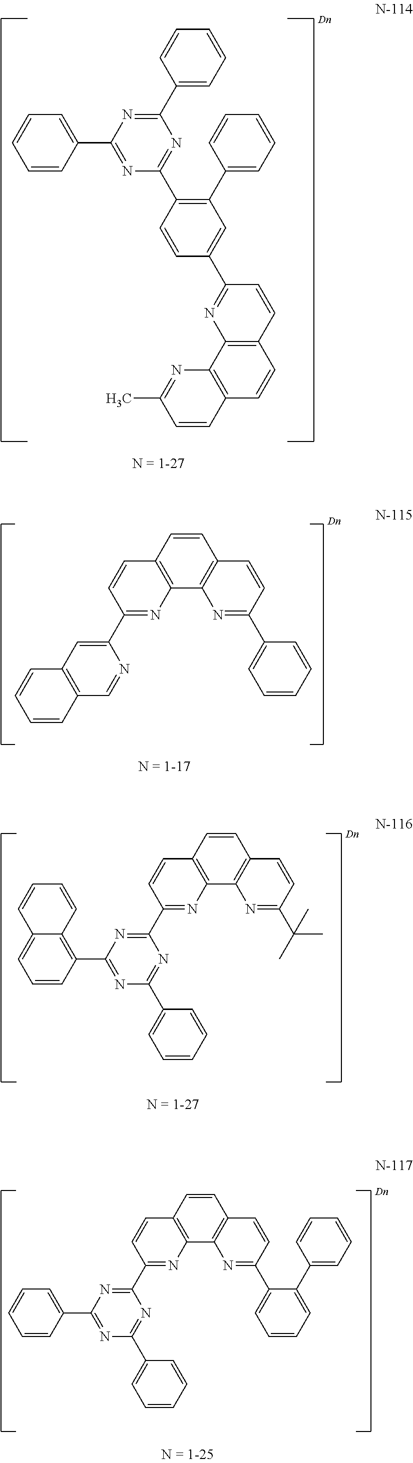

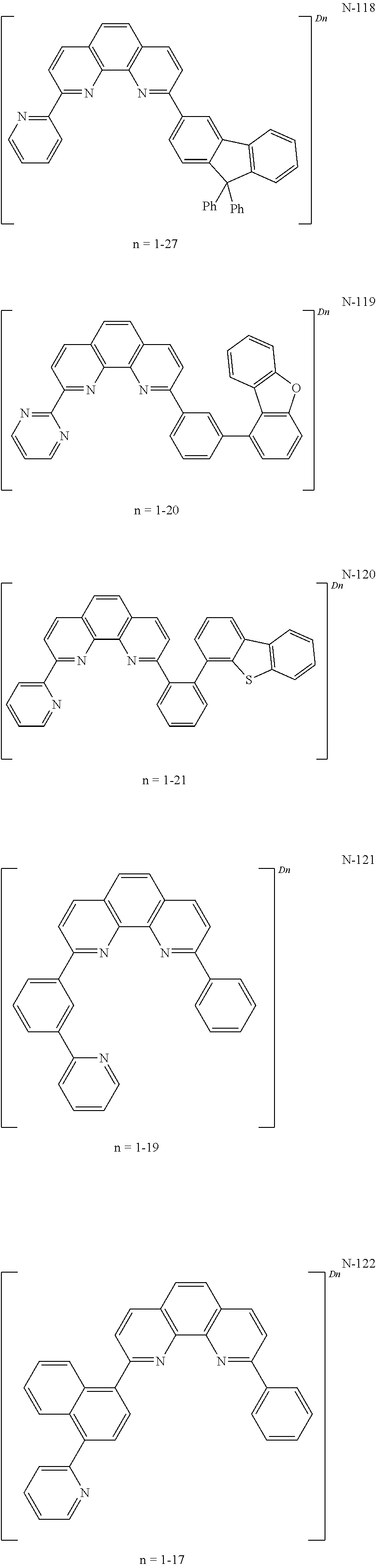

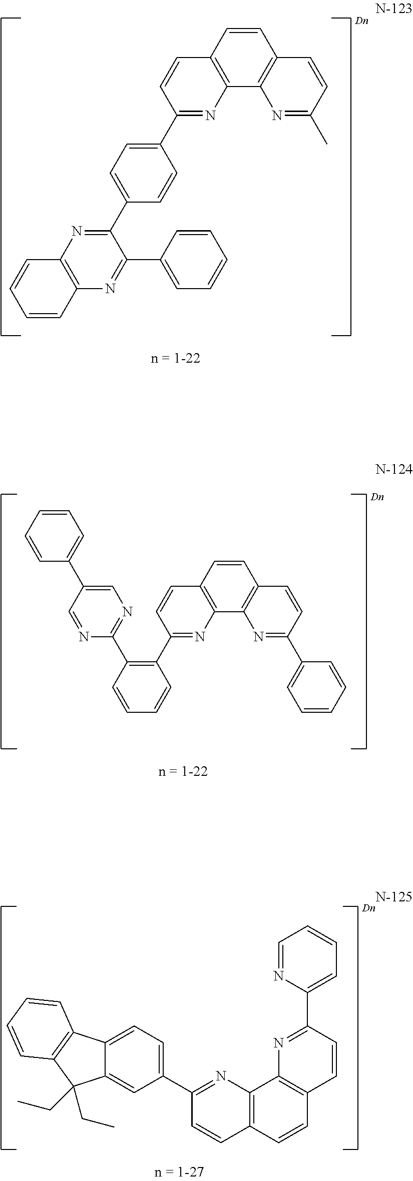

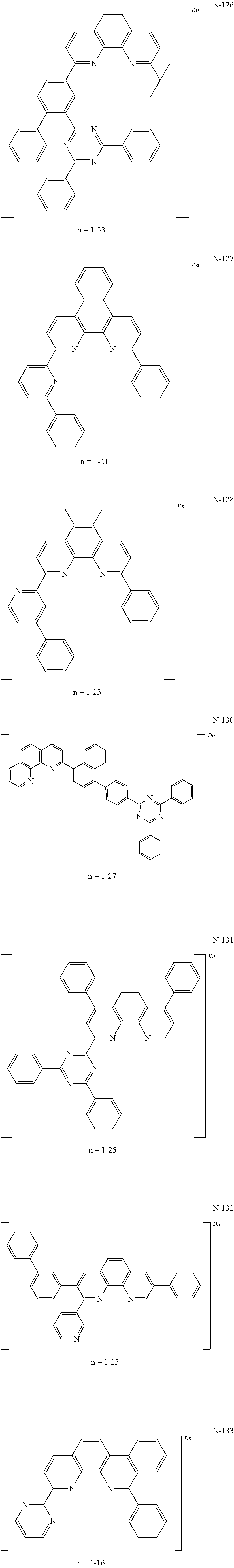

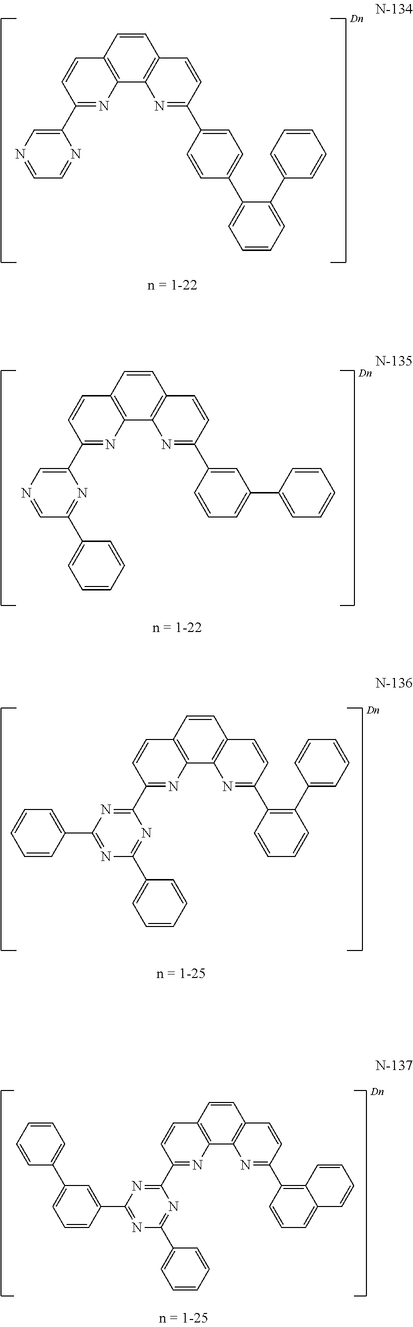

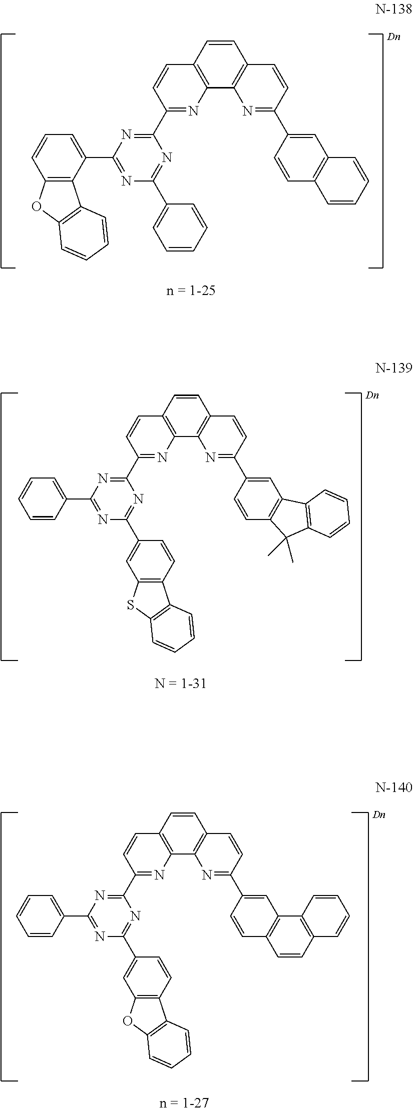

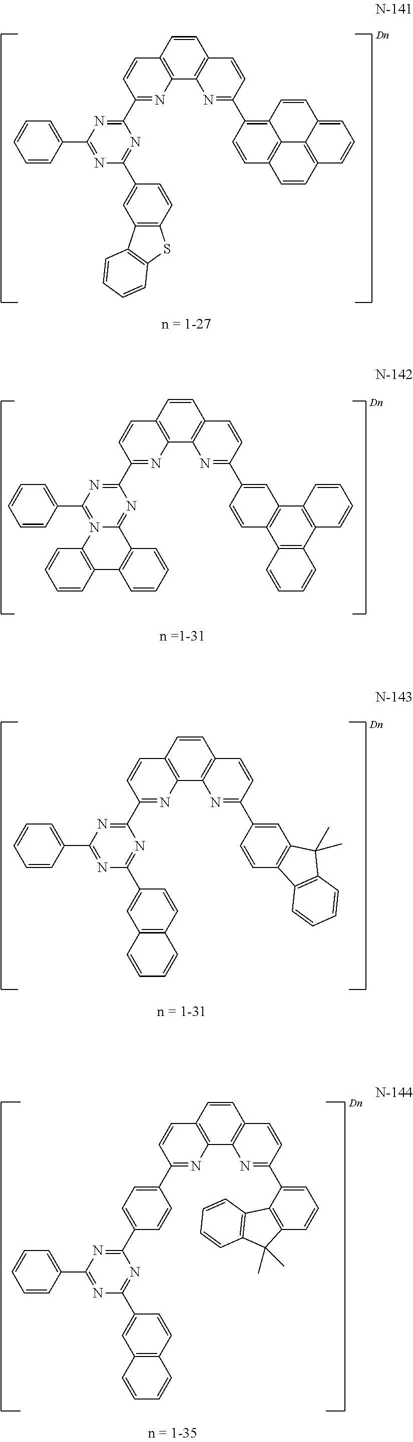

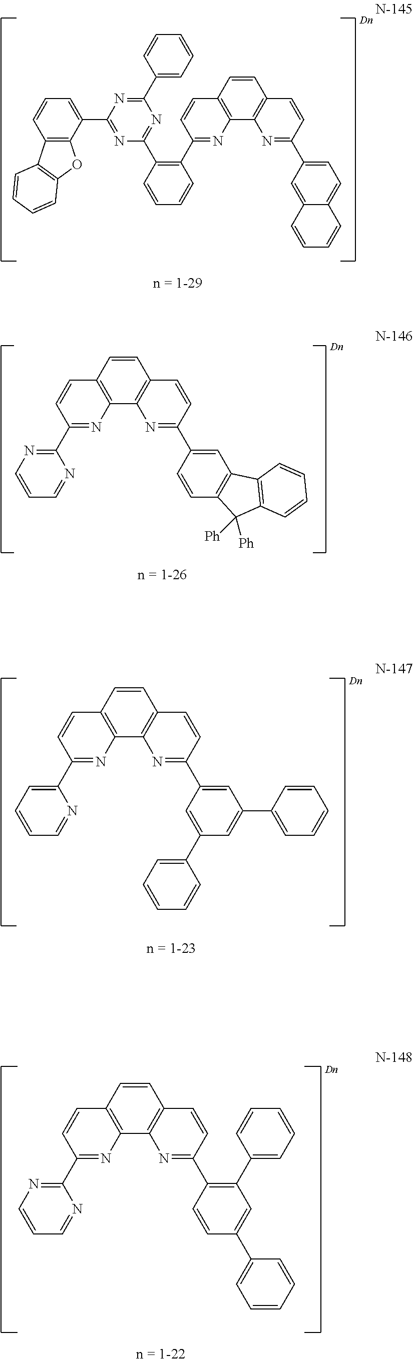

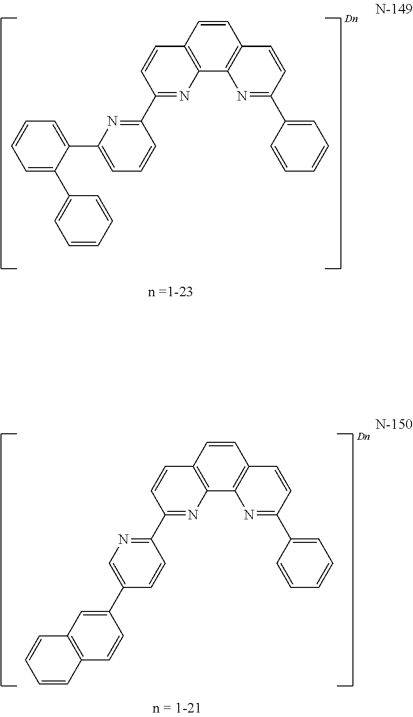

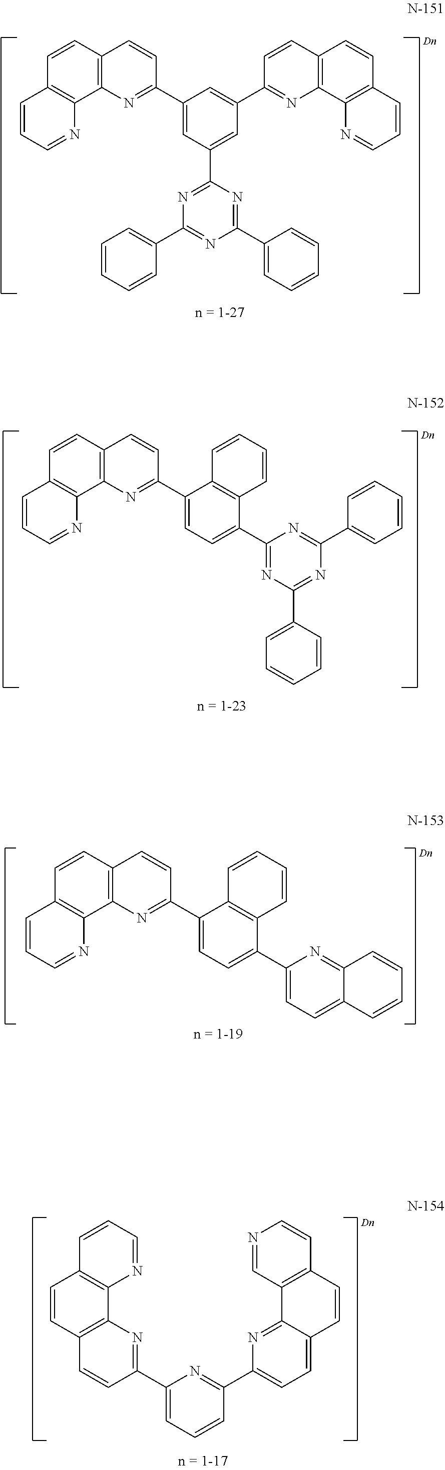

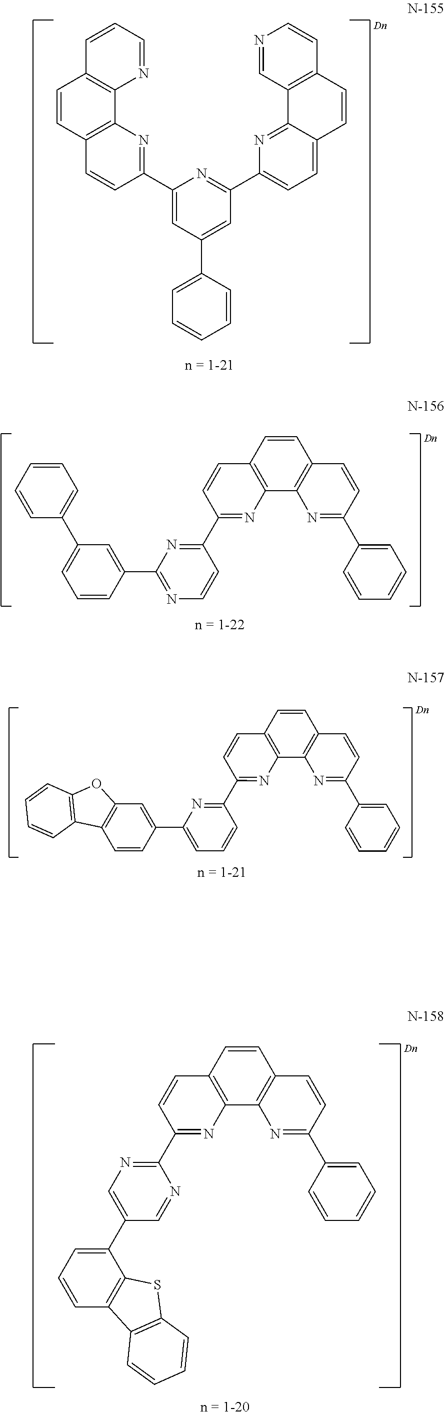

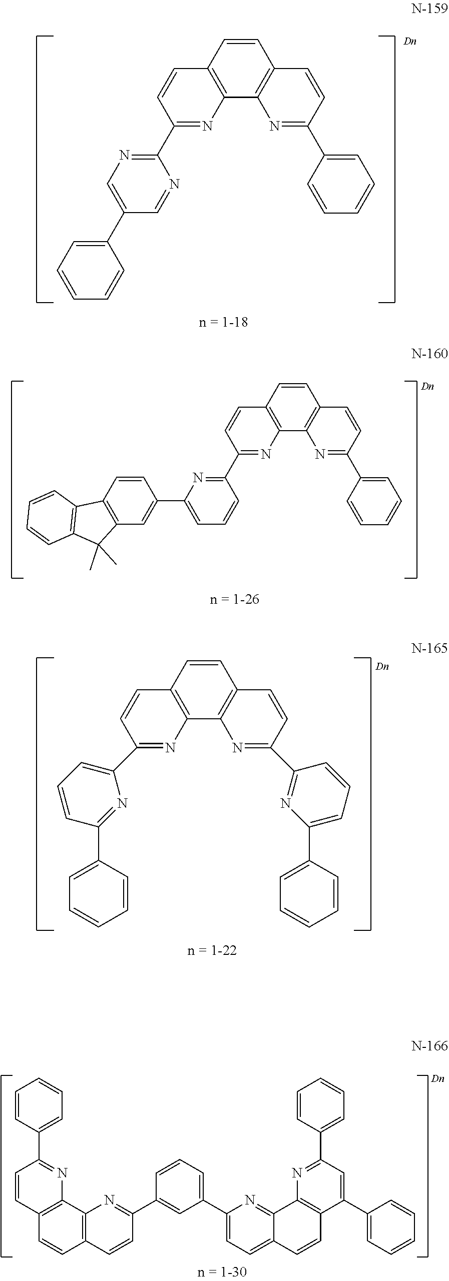

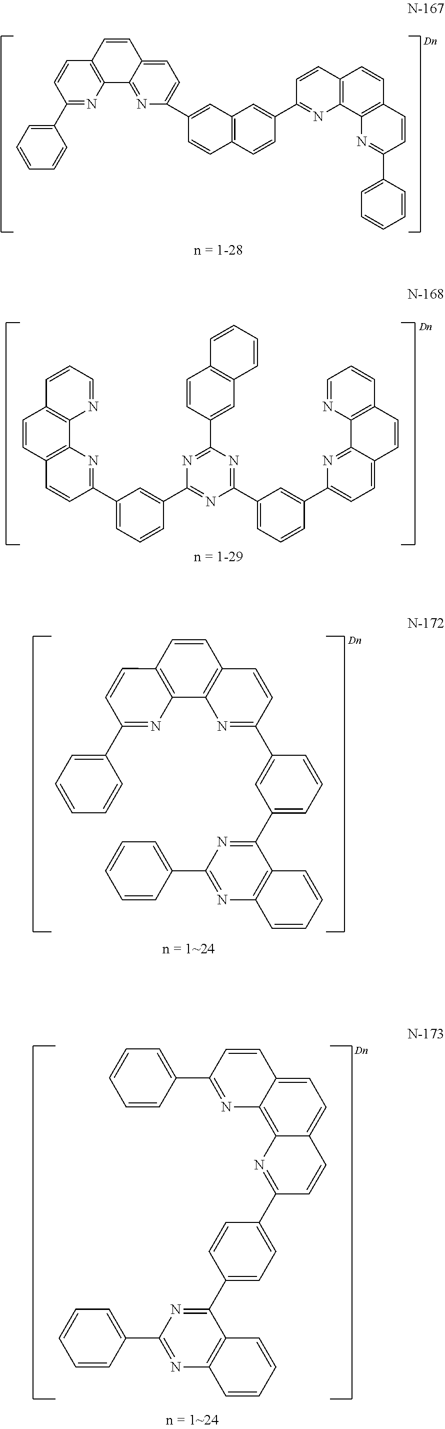

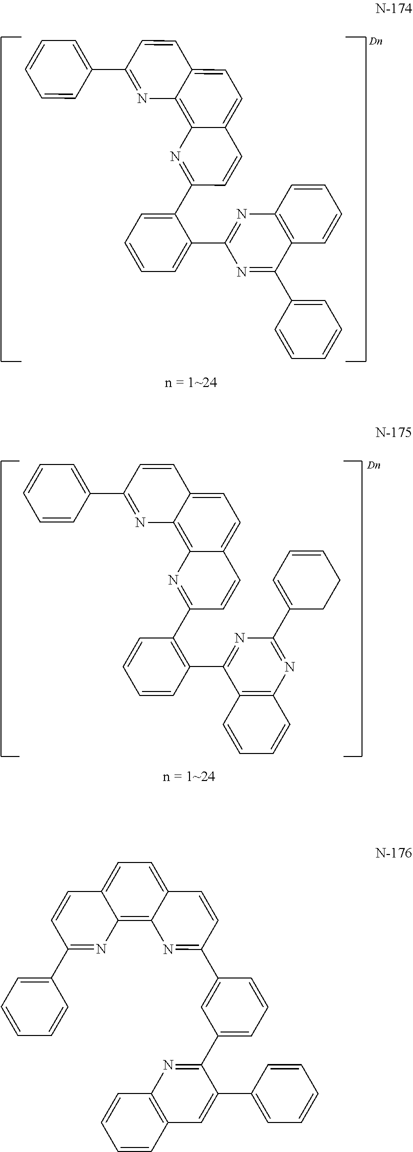

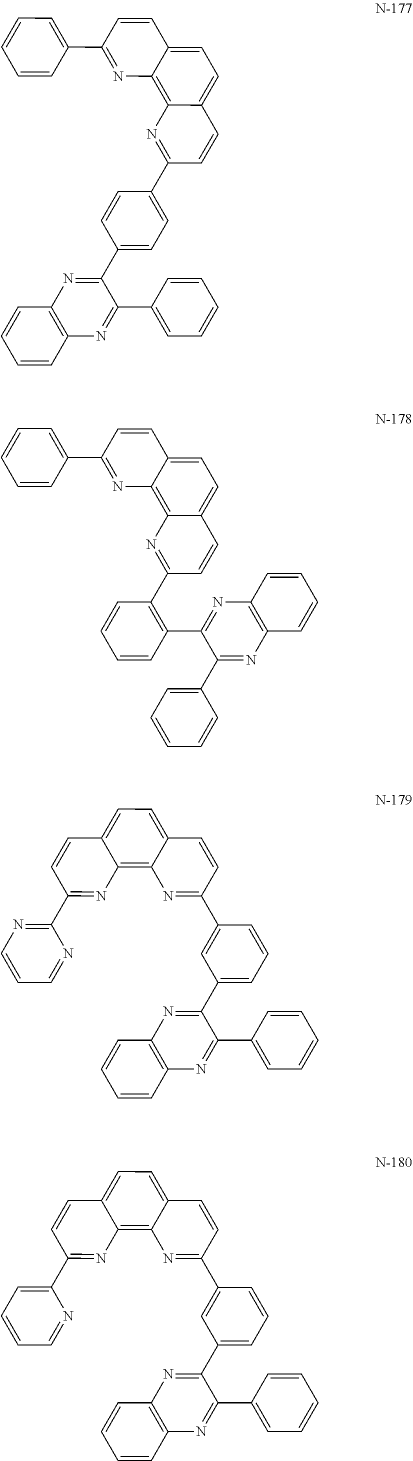 [SMILES: c1ccc(-c2ccc3ccc4ccc(-c5ccc(-c6nc7ccccc7nc6-c6ccccc6)cc5)nc4c3n2)cc1 ; c1ccc(-c2ccc3ccc4ccc(-c5ccccc5-c5nc6ccccc6nc5-c5ccccc5)nc4c3n2)cc1 ; c1ccc(-c2nc3ccccc3nc2-c2cccc(-c3ccc4ccc5ccc(-c6ncccn6)nc5c4n3)c2)cc1 ; c1ccc(-c2nc3ccccc3nc2-c2cccc(-c3ccc4ccc5ccc(-c6ccccn6)nc5c4n3)c2)cc1]

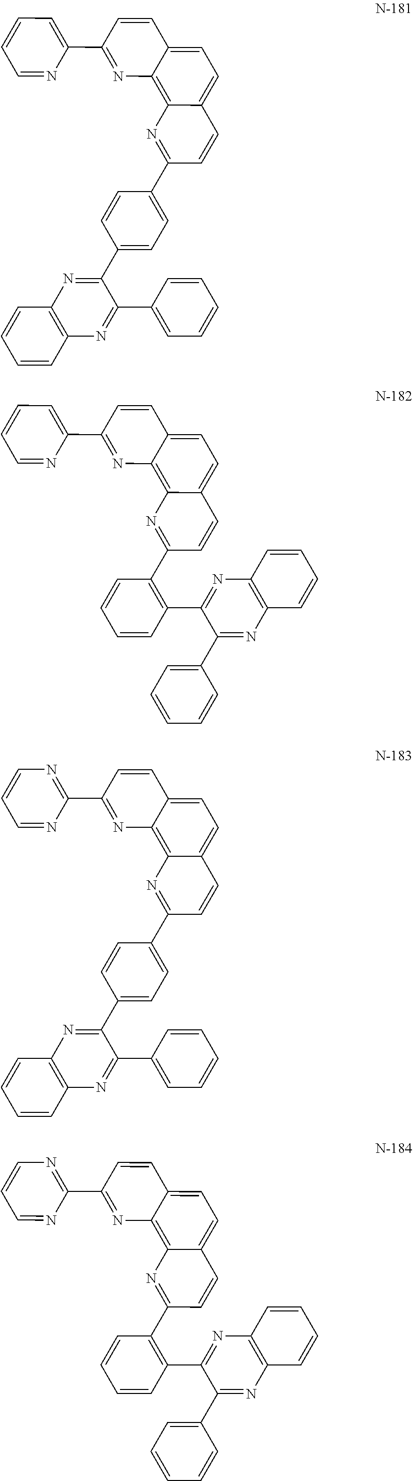

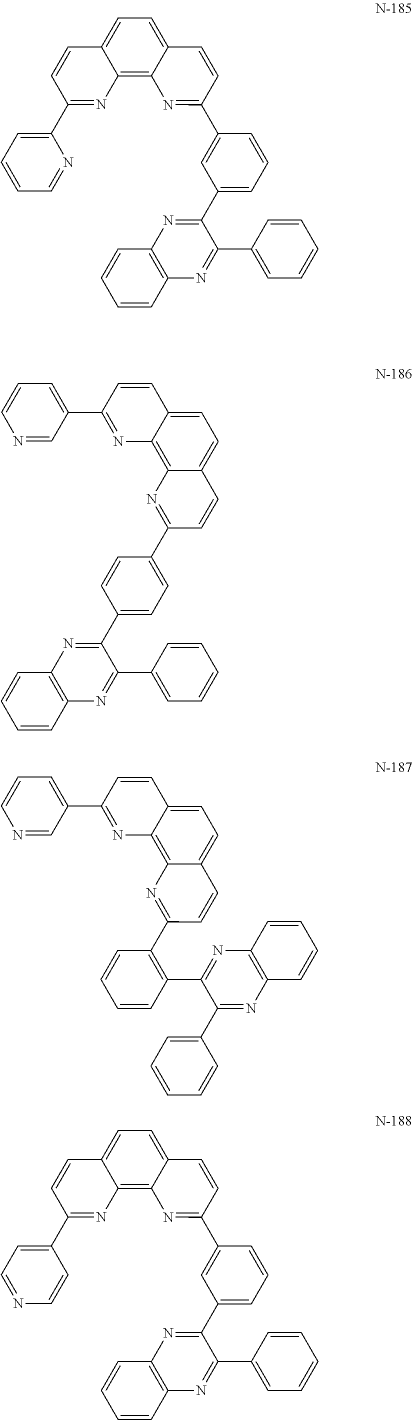

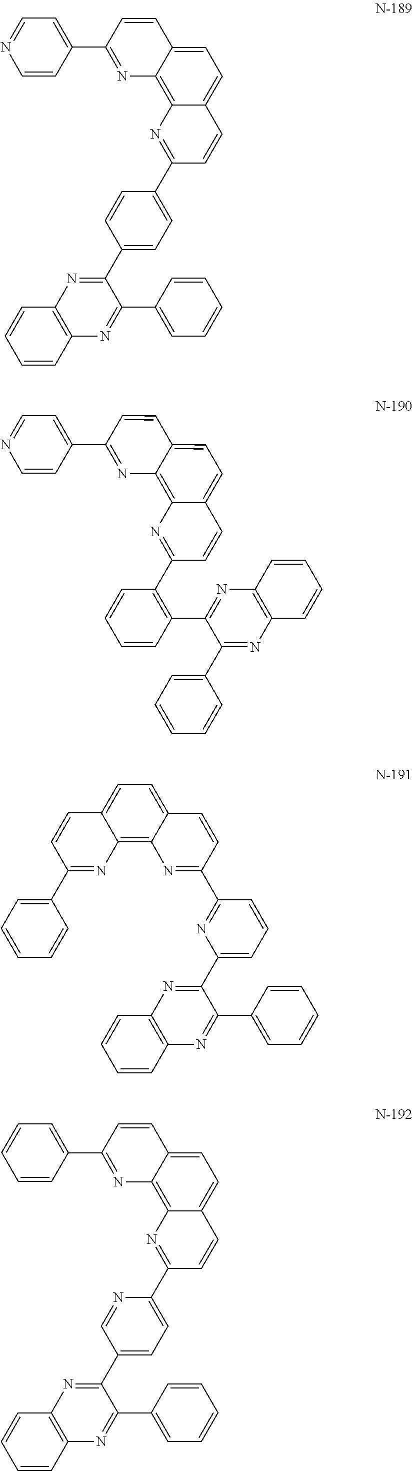 [SMILES: c1ccc(-c2nc3ccccc3nc2-c2ccc(-c3ccc4ccc5ccc(-c6ccncc6)nc5c4n3)cc2)cc1 ; c1ccc(-c2nc3ccccc3nc2-c2ccccc2-c2ccc3ccc4ccc(-c5ccncc5)nc4c3n2)cc1 ; c1ccc(-c2ccc3ccc4ccc(-c5cccc(-c6nc7ccccc7nc6-c6ccccc6)n5)nc4c3n2)cc1 ; c1ccc(-c2ccc3ccc4ccc(-c5ccc(-c6nc7ccccc7nc6-c6ccccc6)cn5)nc4c3n2)cc1]

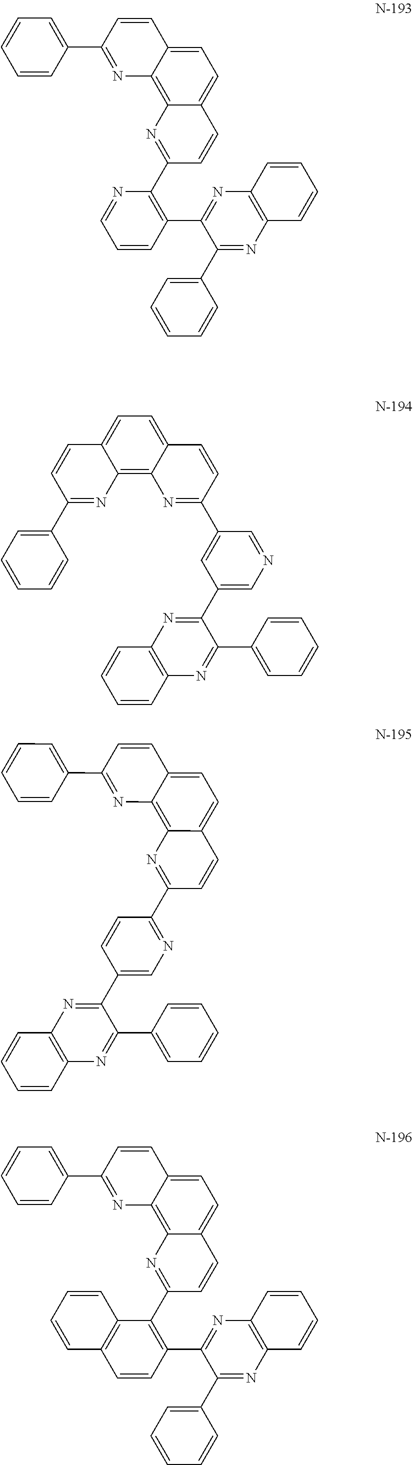

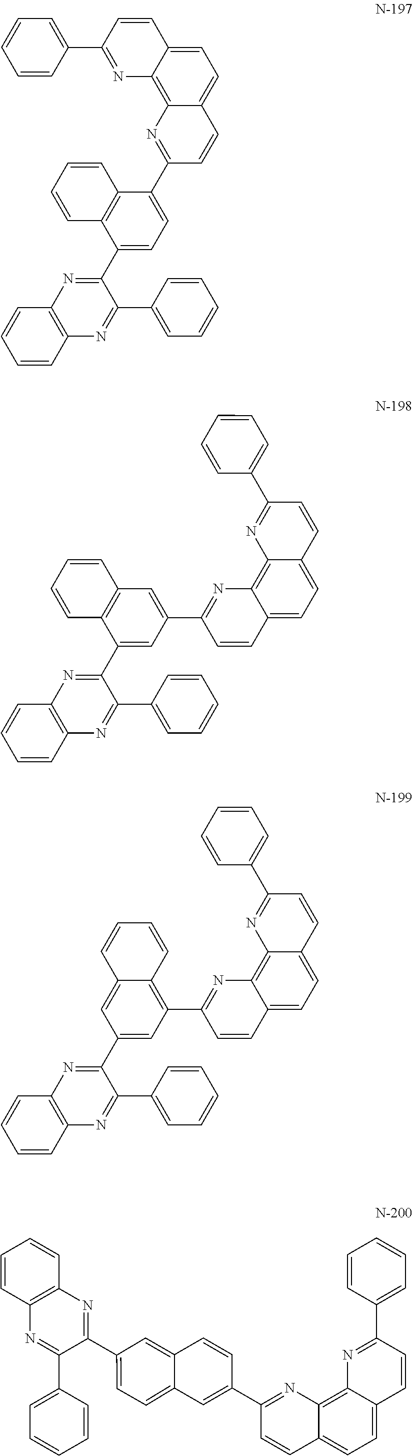 [SMILES: c1ccc(-c2ccc3ccc4ccc(-c5ccc(-c6nc7ccccc7nc6-c6ccccc6)c6ccccc56)nc4c3n2)cc1 ; c1ccc(-c2ccc3ccc4ccc(-c5cc(-c6nc7ccccc7nc6-c6ccccc6)c6ccccc6c5)nc4c3n2)cc1 ; c1ccc(-c2ccc3ccc4ccc(-c5cc(-c6nc7ccccc7nc6-c6ccccc6)cc6ccccc56)nc4c3n2)cc1 ; c1ccc(-c2ccc3ccc4ccc(-c5ccc6cc(-c7nc8ccccc8nc7-c7ccccc7)ccc6c5)nc4c3n2)cc1]

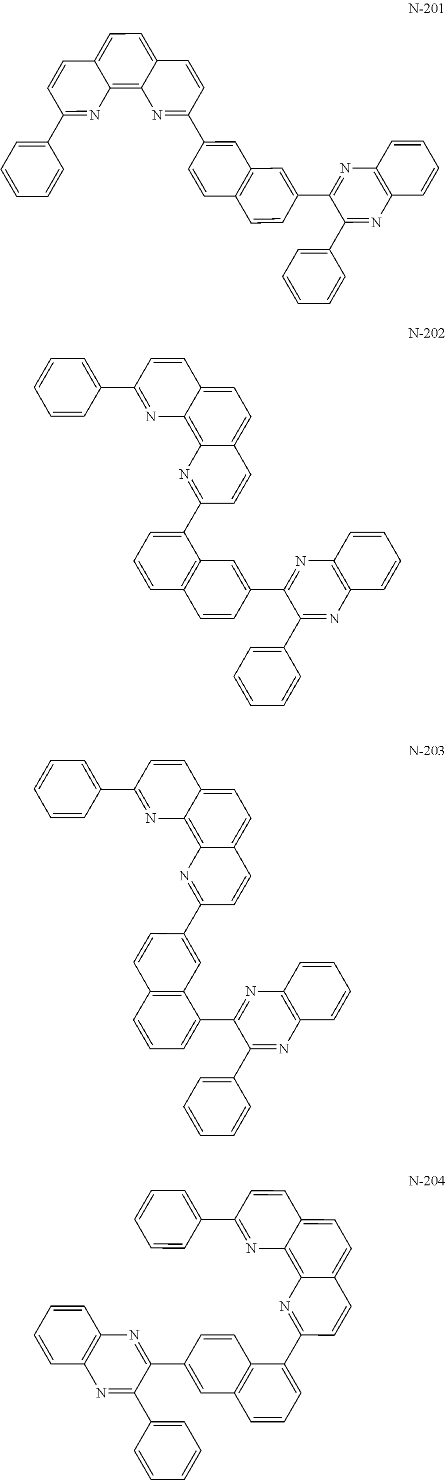 [SMILES: c1ccc(-c2ccc3ccc4ccc(-c5ccc6ccc(-c7nc8ccccc8nc7-c7ccccc7)cc6c5)nc4c3n2)cc1 ; c1ccc(-c2ccc3ccc4ccc(-c5cccc6ccc(-c7nc8ccccc8nc7-c7ccccc7)cc56)nc4c3n2)cc1 ; c1ccc(-c2ccc3ccc4ccc(-c5ccc6cccc(-c7nc8ccccc8nc7-c7ccccc7)c6c5)nc4c3n2)cc1 ; c1ccc(-c2ccc3ccc4ccc(-c5cccc6cc(-c7nc8ccccc8nc7-c7ccccc7)ccc56)nc4c3n2)cc1]

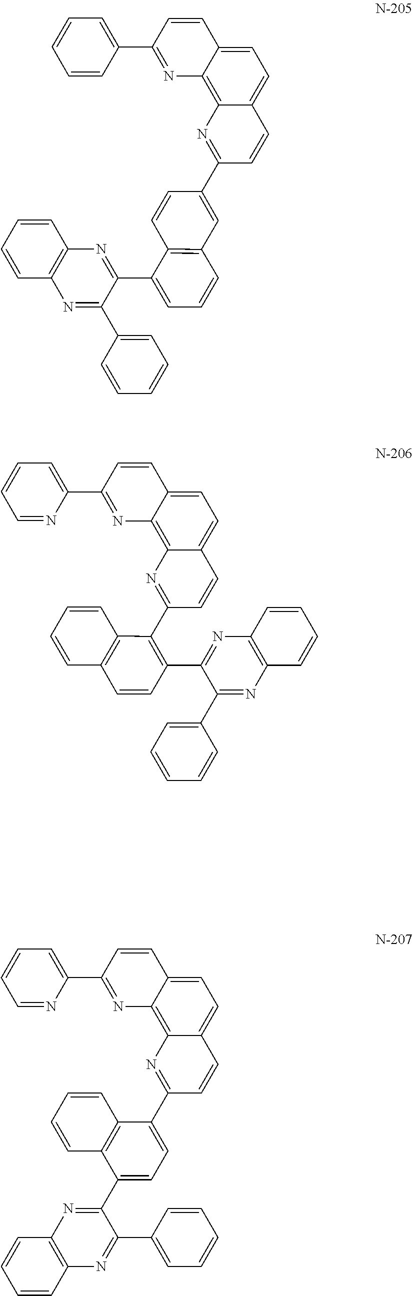 [SMILES: c1ccc(-c2ccc3ccc4ccc(-c5ccc6c(-c7nc8ccccc8nc7-c7ccccc7)cccc6c5)nc4c3n2)cc1 ; c1ccc(-c2nc3ccccc3nc2-c2ccc3ccccc3c2-c2ccc3ccc4ccc(-c5ccccn5)nc4c3n2)cc1 ; c1ccc(-c2nc3ccccc3nc2-c2ccc(-c3ccc4ccc5ccc(-c6ccccn6)nc5c4n3)c3ccccc23)cc1]

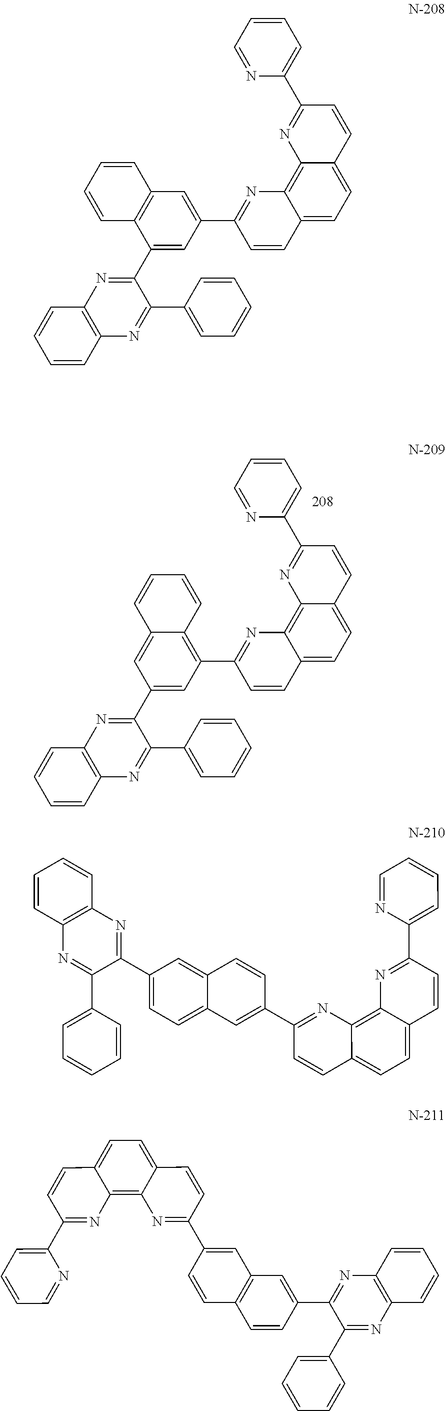 [SMILES: c1ccc(-c2nc3ccccc3nc2-c2cc(-c3ccc4ccc5ccc(-c6ccccn6)nc5c4n3)cc3ccccc23)cc1 ; c1ccc(-c2nc3ccccc3nc2-c2cc(-c3ccc4ccc5ccc(-c6ccccn6)nc5c4n3)c3ccccc3c2)cc1 ; c1ccc(-c2nc3ccccc3nc2-c2ccc3cc(-c4ccc5ccc6ccc(-c7ccccn7)nc6c5n4)ccc3c2)cc1 ; c1ccc(-c2nc3ccccc3nc2-c2ccc3ccc(-c4ccc5ccc6ccc(-c7ccccn7)nc6c5n4)cc3c2)cc1]

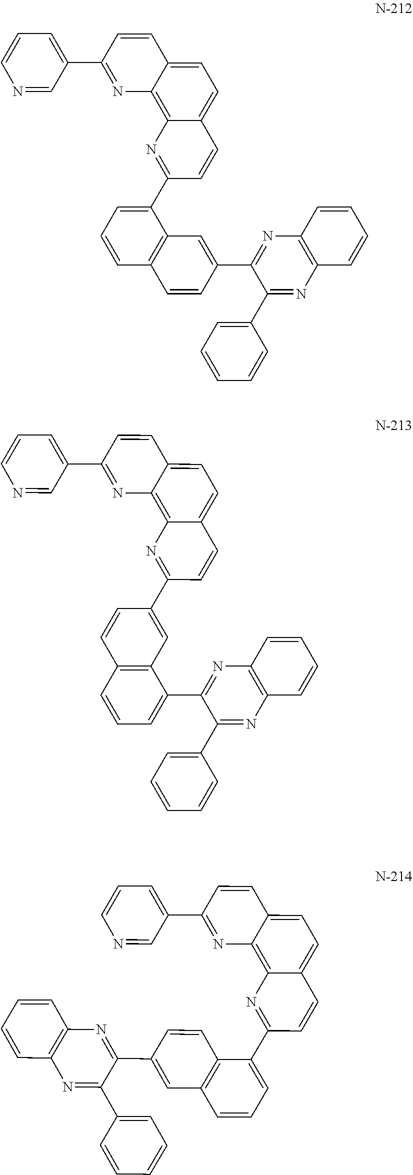 [SMILES: c1ccc(-c2nc3ccccc3nc2-c2ccc3cccc(-c4ccc5ccc6ccc(-c7cccnc7)nc6c5n4)c3c2)cc1 ; c1ccc(-c2nc3ccccc3nc2-c2cccc3ccc(-c4ccc5ccc6ccc(-c7cccnc7)nc6c5n4)cc23)cc1 ; c1ccc(-c2nc3ccccc3nc2-c2ccc3c(-c4ccc5ccc6ccc(-c7cccnc7)nc6c5n4)cccc3c2)cc1]

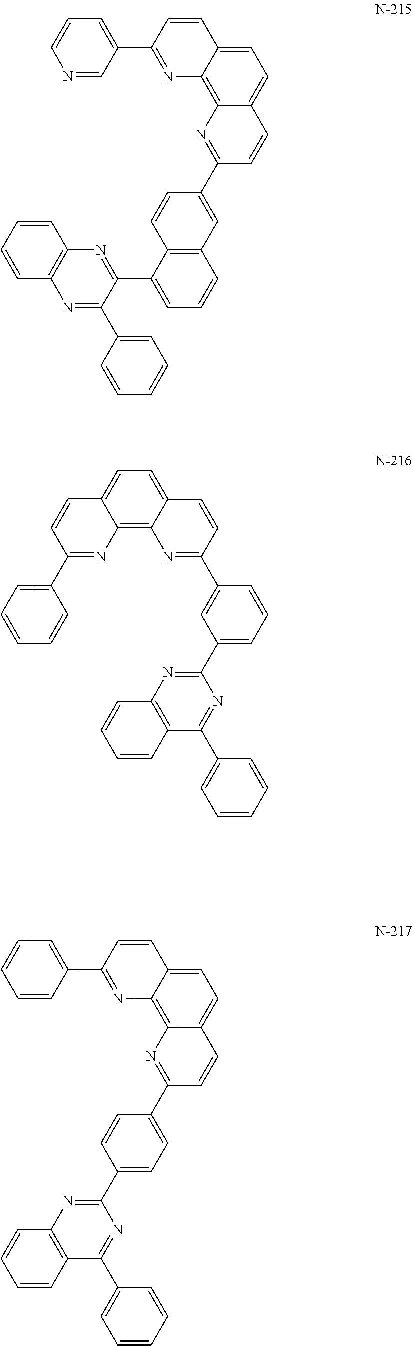 [SMILES: c1ccc(-c2nc3ccccc3nc2-c2cccc3cc(-c4ccc5ccc6ccc(-c7cccnc7)nc6c5n4)ccc23)cc1 ; c1ccc(-c2ccc3ccc4ccc(-c5cccc(-c6nc(-c7ccccc7)c7ccccc7n6)c5)nc4c3n2)cc1 ; c1ccc(-c2ccc3ccc4ccc(-c5ccc(-c6nc(-c7ccccc7)c7ccccc7n6)cc5)nc4c3n2)cc1]

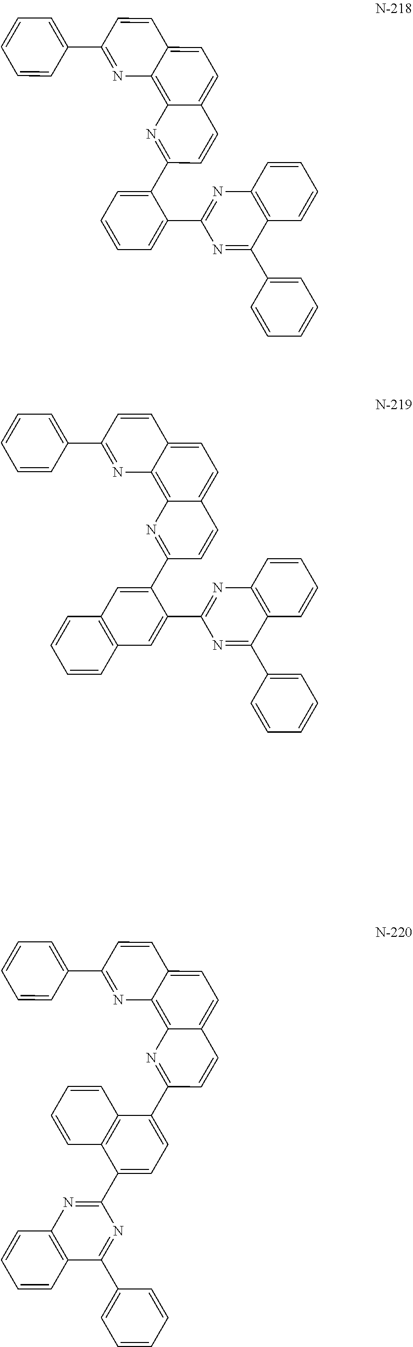 [SMILES: c1ccc(-c2ccc3ccc4ccc(-c5ccccc5-c5nc(-c6ccccc6)c6ccccc6n5)nc4c3n2)cc1 ; c1ccc(-c2ccc3ccc4ccc(-c5cc6ccccc6cc5-c5nc(-c6ccccc6)c6ccccc6n5)nc4c3n2)cc1 ; c1ccc(-c2ccc3ccc4ccc(-c5ccc(-c6nc(-c7ccccc7)c7ccccc7n6)c6ccccc56)nc4c3n2)cc1]

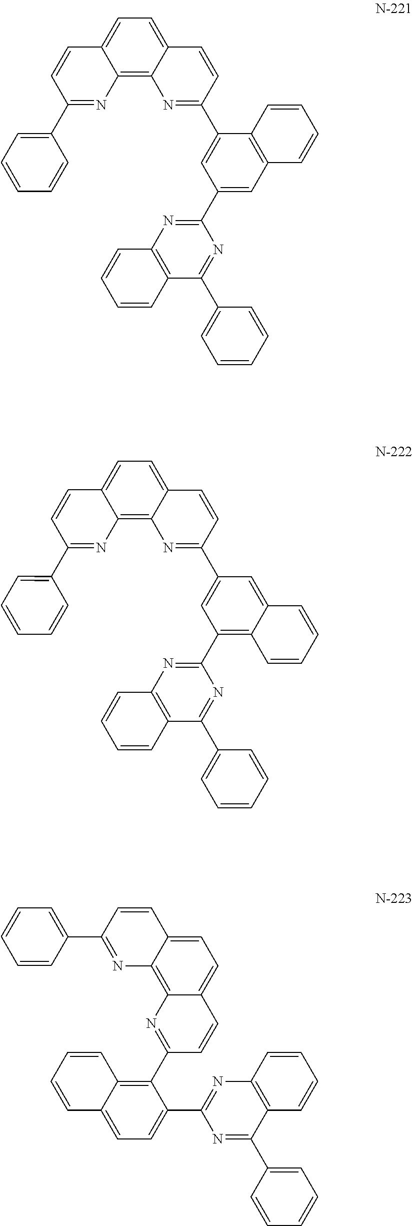 [SMILES: c1ccc(-c2ccc3ccc4ccc(-c5cc(-c6nc(-c7ccccc7)c7ccccc7n6)cc6ccccc56)nc4c3n2)cc1 ; c1ccc(-c2ccc3ccc4ccc(-c5cc(-c6nc(-c7ccccc7)c7ccccc7n6)c6ccccc6c5)nc4c3n2)cc1 ; c1ccc(-c2ccc3ccc4ccc(-c5c(-c6nc(-c7ccccc7)c7ccccc7n6)ccc6ccccc56)nc4c3n2)cc1]

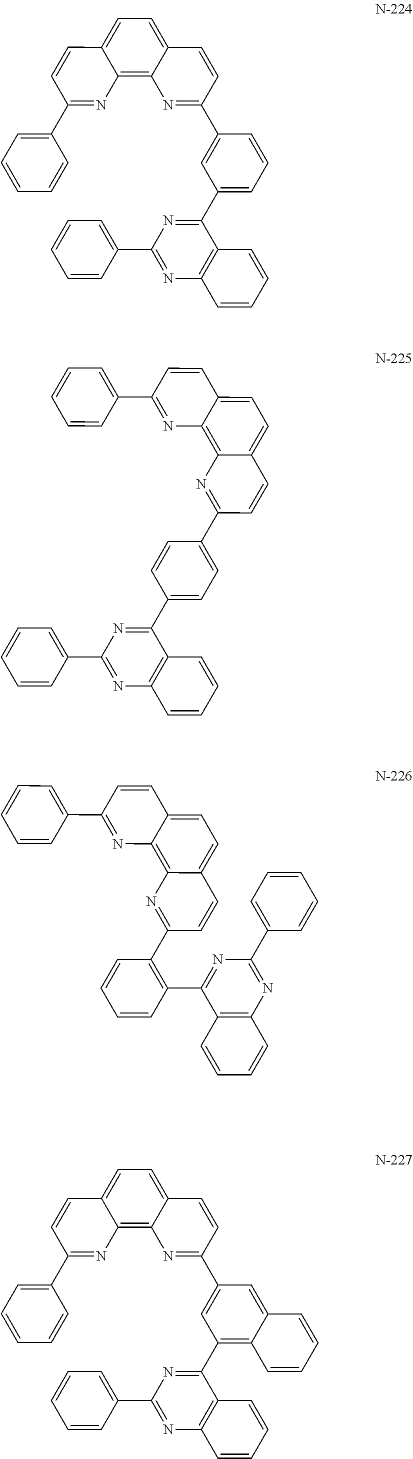 [SMILES: c1ccc(-c2ccc3ccc4ccc(-c5cccc(-c6nc(-c7ccccc7)nc7ccccc67)c5)nc4c3n2)cc1 ; c1ccc(-c2ccc3ccc4ccc(-c5ccc(-c6nc(-c7ccccc7)nc7ccccc67)cc5)nc4c3n2)cc1 ; c1ccc(-c2ccc3ccc4ccc(-c5ccccc5-c5nc(-c6ccccc6)nc6ccccc56)nc4c3n2)cc1 ; c1ccc(-c2ccc3ccc4ccc(-c5cc(-c6nc(-c7ccccc7)nc7ccccc67)c6ccccc6c5)nc4c3n2)cc1]

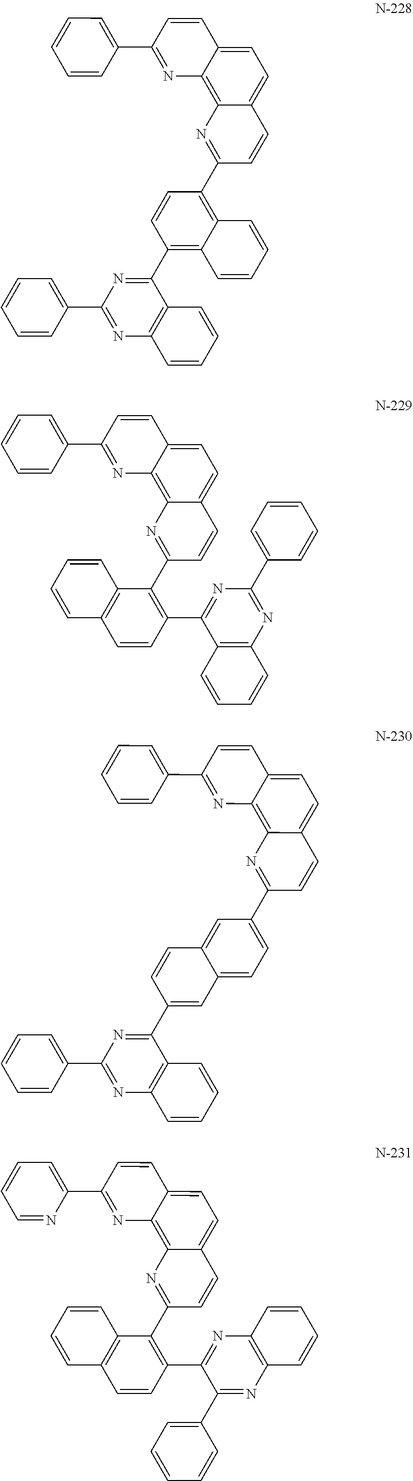

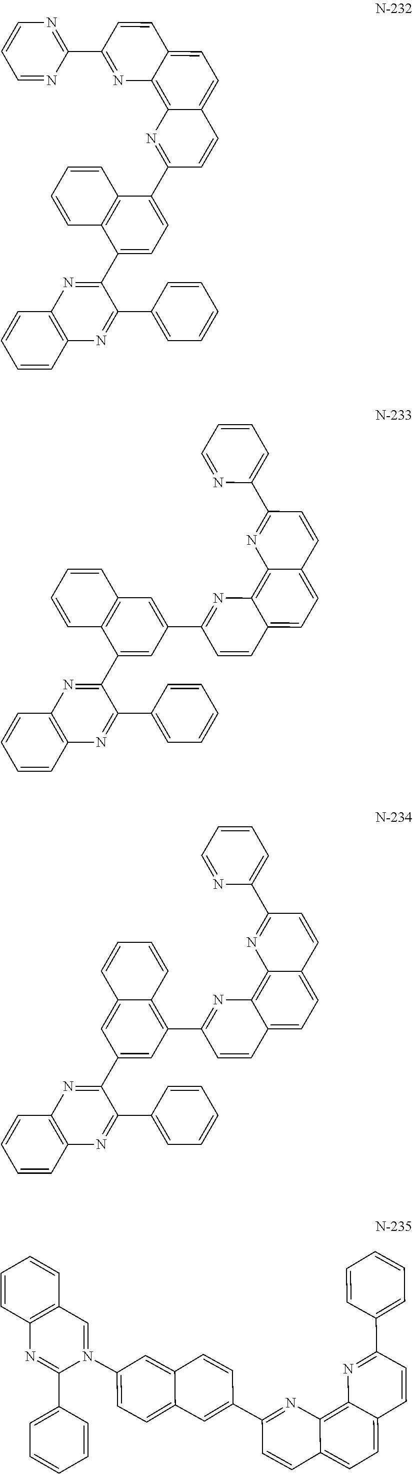

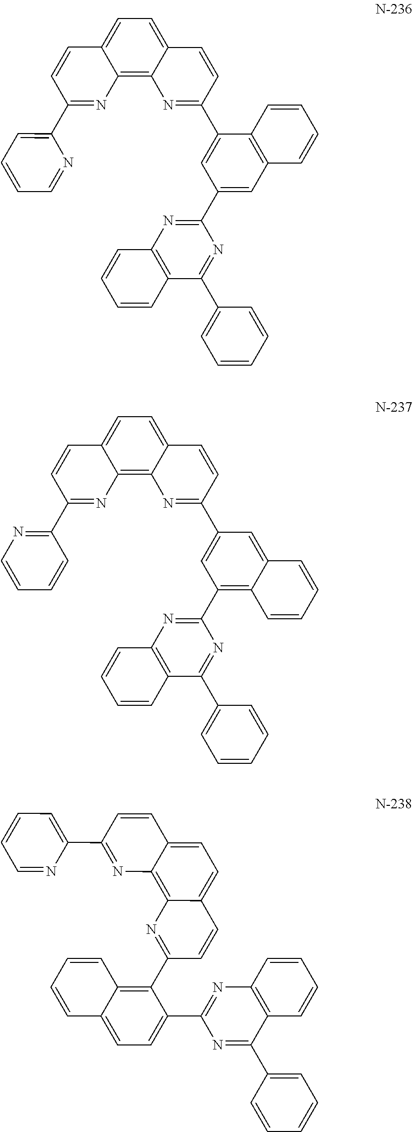 [SMILES: c1ccc(-c2nc(-c3cc(-c4ccc5ccc6ccc(-c7ccccn7)nc6c5n4)c4ccccc4c3)nc3ccccc23)cc1 ; c1ccc(-c2nc(-c3cc(-c4ccc5ccc6ccc(-c7ccccn7)nc6c5n4)cc4ccccc34)nc3ccccc23)cc1 ; c1ccc(-c2nc(-c3ccc4ccccc4c3-c3ccc4ccc5ccc(-c6ccccn6)nc5c4n3)nc3ccccc23)cc1]

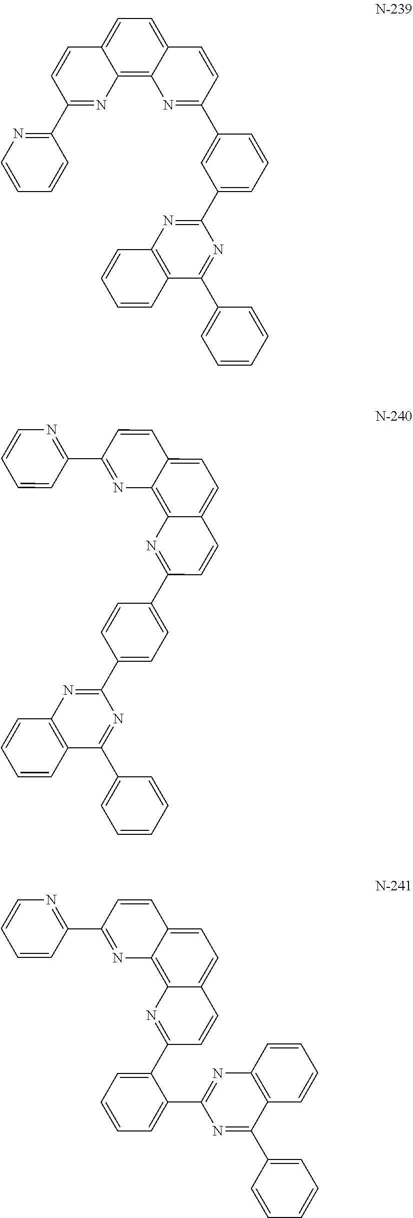 [SMILES: c1ccc(-c2nc(-c3cccc(-c4ccc5ccc6ccc(-c7ccccn7)nc6c5n4)c3)nc3ccccc23)cc1 ; c1ccc(-c2nc(-c3ccc(-c4ccc5ccc6ccc(-c7ccccn7)nc6c5n4)cc3)nc3ccccc23)cc1 ; c1ccc(-c2nc(-c3ccccc3-c3ccc4ccc5ccc(-c6ccccn6)nc5c4n3)nc3ccccc23)cc1]

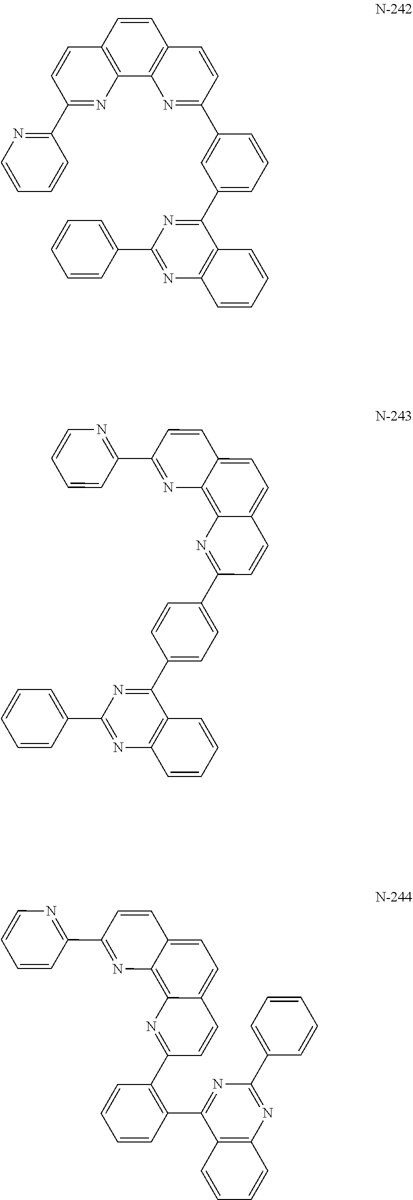 [SMILES: c1ccc(-c2nc(-c3cccc(-c4ccc5ccc6ccc(-c7ccccn7)nc6c5n4)c3)c3ccccc3n2)cc1 ; c1ccc(-c2nc(-c3ccc(-c4ccc5ccc6ccc(-c7ccccn7)nc6c5n4)cc3)c3ccccc3n2)cc1 ; c1ccc(-c2nc(-c3ccccc3-c3ccc4ccc5ccc(-c6ccccn6)nc5c4n3)c3ccccc3n2)cc1]

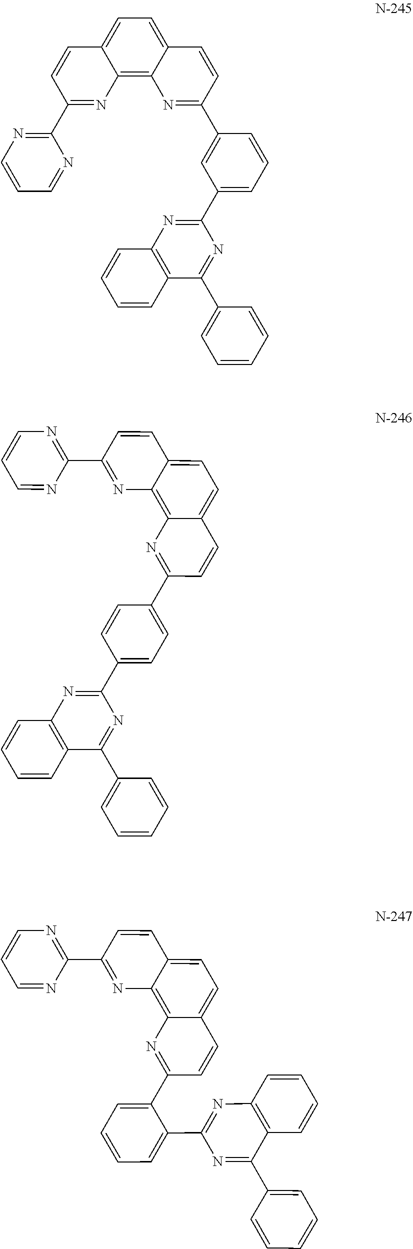 [SMILES: c1ccc(-c2nc(-c3cccc(-c4ccc5ccc6ccc(-c7ncccn7)nc6c5n4)c3)nc3ccccc23)cc1 ; c1ccc(-c2nc(-c3ccc(-c4ccc5ccc6ccc(-c7ncccn7)nc6c5n4)cc3)nc3ccccc23)cc1 ; c1ccc(-c2nc(-c3ccccc3-c3ccc4ccc5ccc(-c6ncccn6)nc5c4n3)nc3ccccc23)cc1]

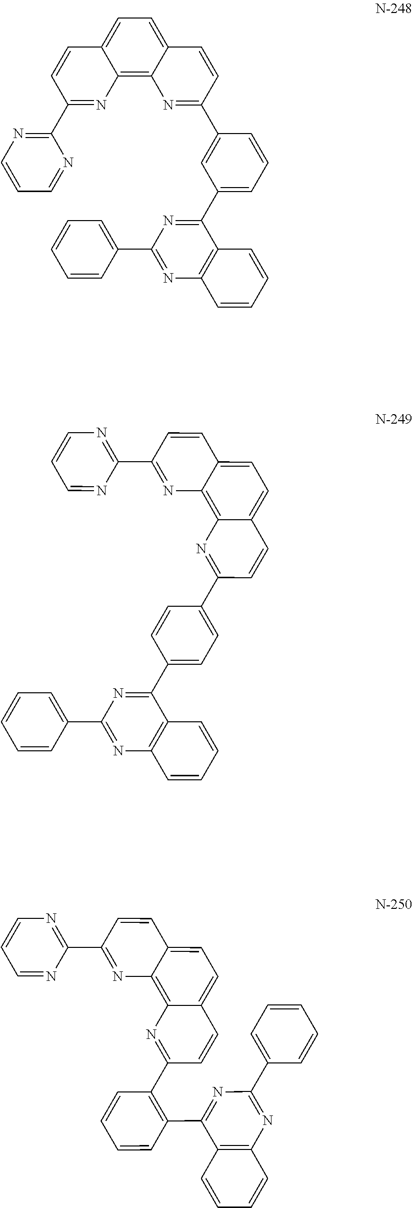 [SMILES: c1ccc(-c2nc(-c3cccc(-c4ccc5ccc6ccc(-c7ncccn7)nc6c5n4)c3)c3ccccc3n2)cc1 ; c1ccc(-c2nc(-c3ccc(-c4ccc5ccc6ccc(-c7ncccn7)nc6c5n4)cc3)c3ccccc3n2)cc1 ; c1ccc(-c2nc(-c3ccccc3-c3ccc4ccc5ccc(-c6ncccn6)nc5c4n3)c3ccccc3n2)cc1]

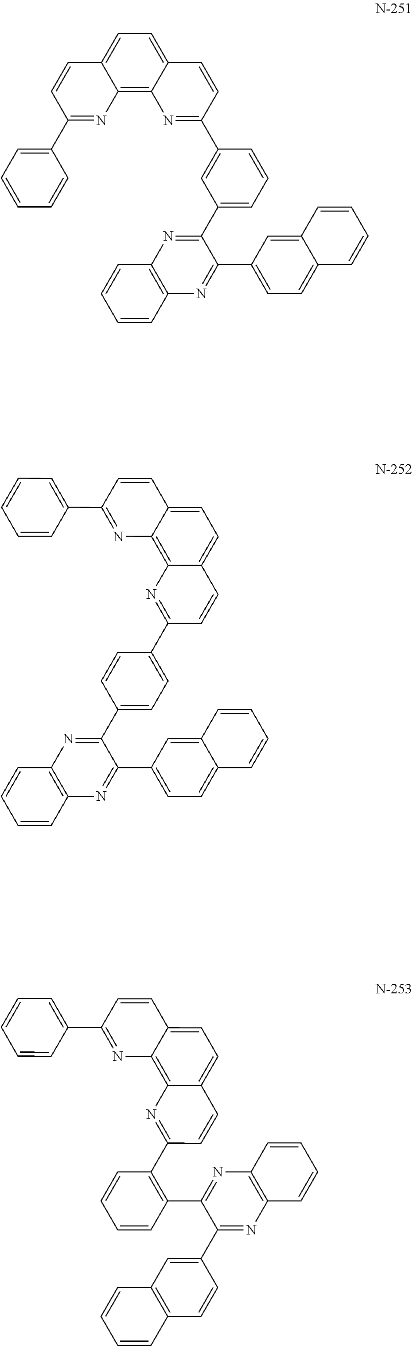 [SMILES: c1ccc(-c2ccc3ccc4ccc(-c5cccc(-c6nc7ccccc7nc6-c6ccc7ccccc7c6)c5)nc4c3n2)cc1 ; c1ccc(-c2ccc3ccc4ccc(-c5ccc(-c6nc7ccccc7nc6-c6ccc7ccccc7c6)cc5)nc4c3n2)cc1 ; c1ccc(-c2ccc3ccc4ccc(-c5ccccc5-c5nc6ccccc6nc5-c5ccc6ccccc6c5)nc4c3n2)cc1]

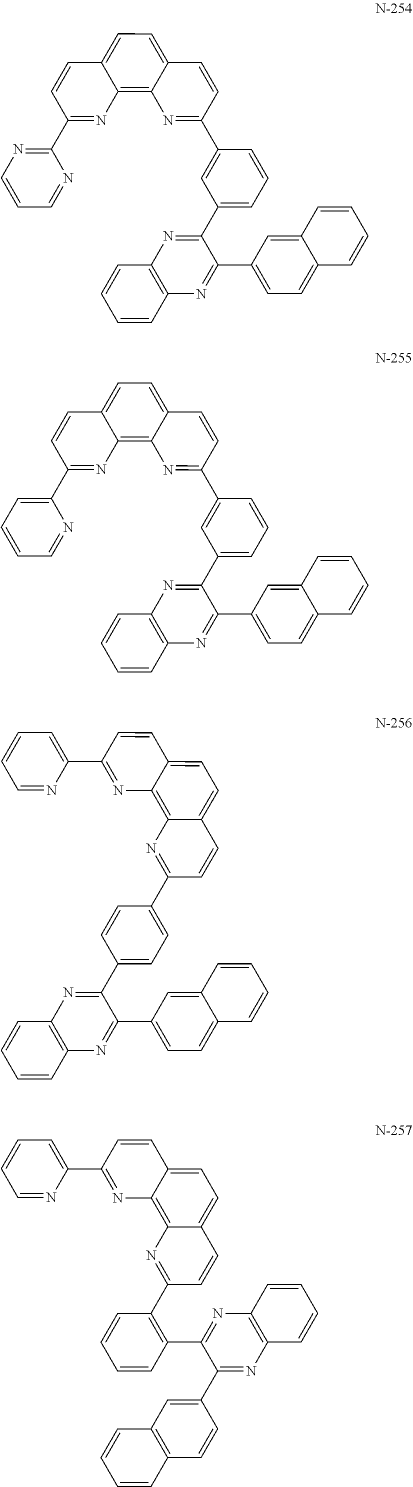 [SMILES: c1cnc(-c2ccc3ccc4ccc(-c5cccc(-c6nc7ccccc7nc6-c6ccc7ccccc7c6)c5)nc4c3n2)nc1 ; c1ccc(-c2ccc3ccc4ccc(-c5cccc(-c6nc7ccccc7nc6-c6ccc7ccccc7c6)c5)nc4c3n2)nc1 ; c1ccc(-c2ccc3ccc4ccc(-c5ccc(-c6nc7ccccc7nc6-c6ccc7ccccc7c6)cc5)nc4c3n2)nc1 ; c1ccc(-c2ccc3ccc4ccc(-c5ccccc5-c5nc6ccccc6nc5-c5ccc6ccccc6c5)nc4c3n2)nc1]

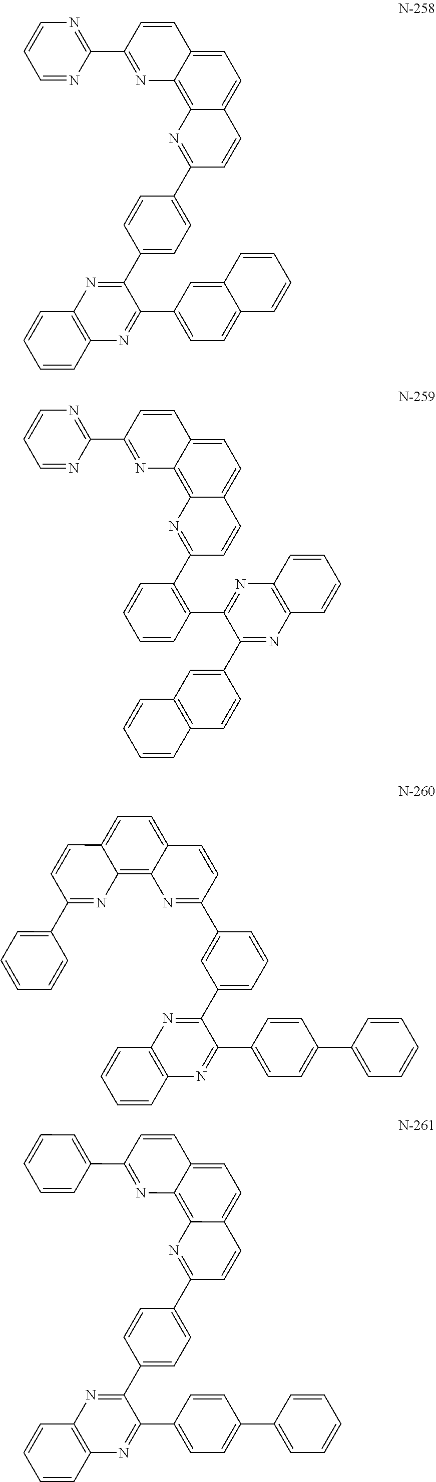

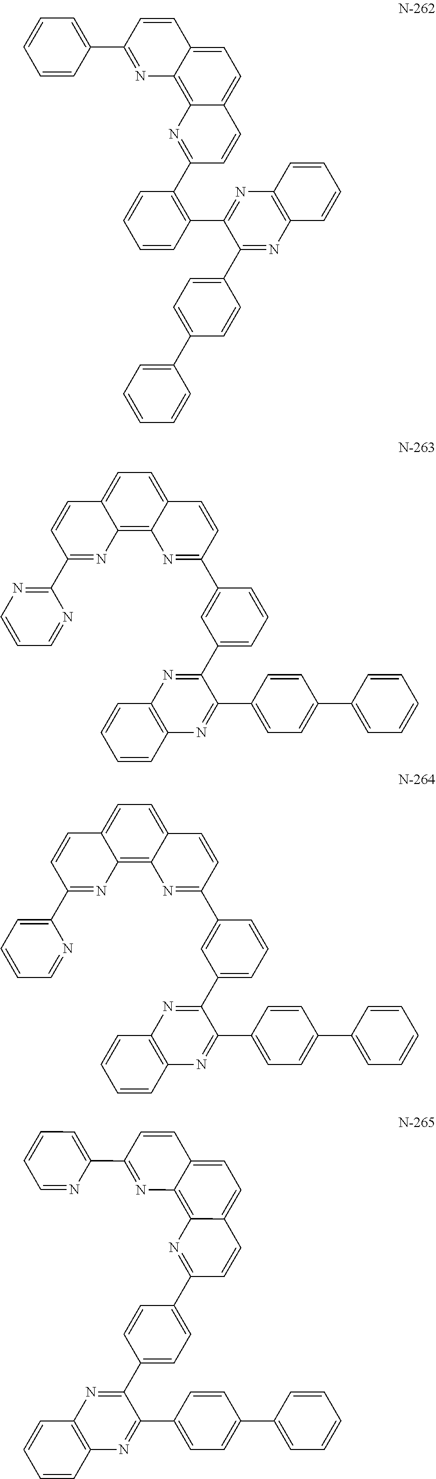

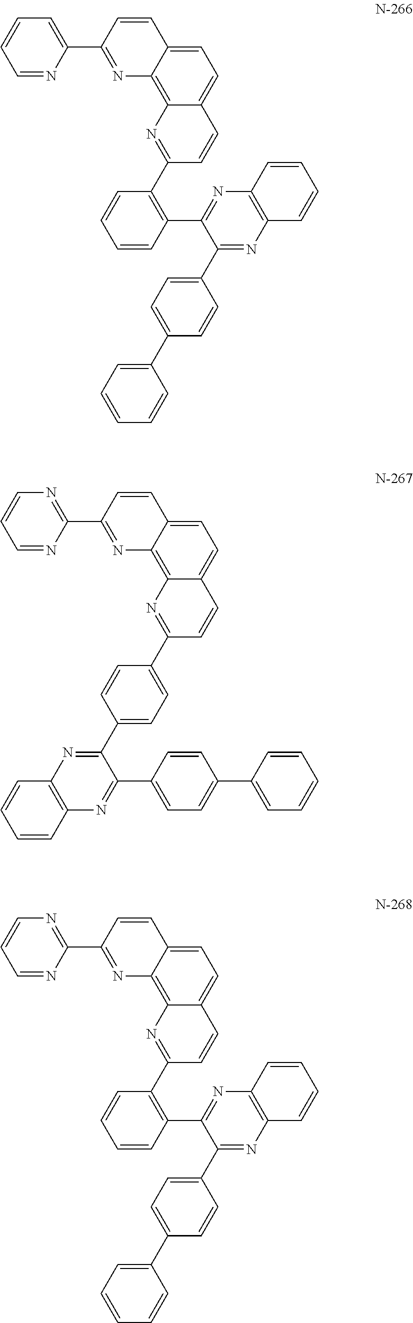 [SMILES: c1ccc(-c2ccc(-c3nc4ccccc4nc3-c3ccccc3-c3ccc4ccc5ccc(-c6ccccn6)nc5c4n3)cc2)cc1 ; c1ccc(-c2ccc(-c3nc4ccccc4nc3-c3ccc(-c4ccc5ccc6ccc(-c7ncccn7)nc6c5n4)cc3)cc2)cc1 ; c1ccc(-c2ccc(-c3nc4ccccc4nc3-c3ccccc3-c3ccc4ccc5ccc(-c6ncccn6)nc5c4n3)cc2)cc1]

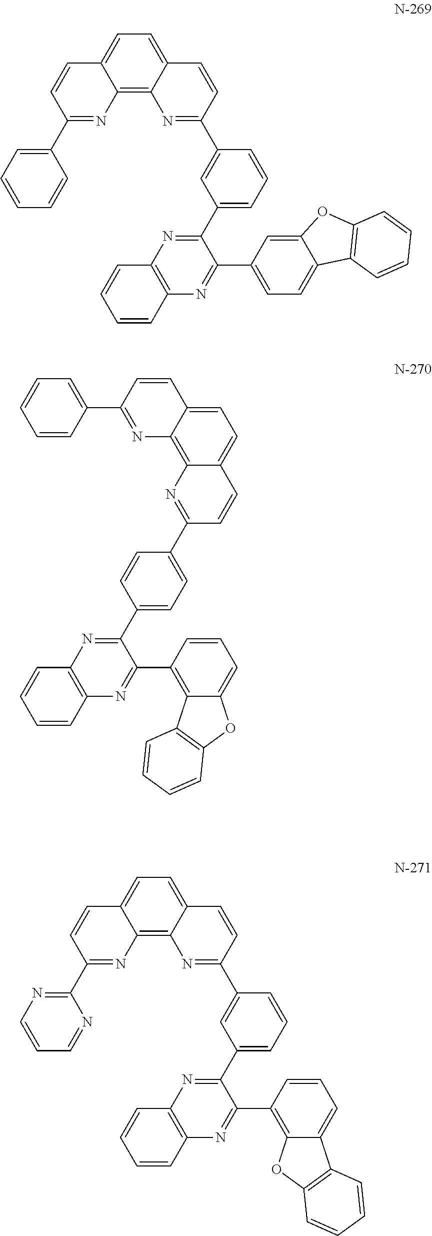 [SMILES: c1ccc(-c2ccc3ccc4ccc(-c5cccc(-c6nc7ccccc7nc6-c6ccc7c(c6)oc6ccccc67)c5)nc4c3n2)cc1 ; c1ccc(-c2ccc3ccc4ccc(-c5ccc(-c6nc7ccccc7nc6-c6cccc7oc8ccccc8c67)cc5)nc4c3n2)cc1 ; c1cnc(-c2ccc3ccc4ccc(-c5cccc(-c6nc7ccccc7nc6-c6cccc7c6oc6ccccc67)c5)nc4c3n2)nc1]

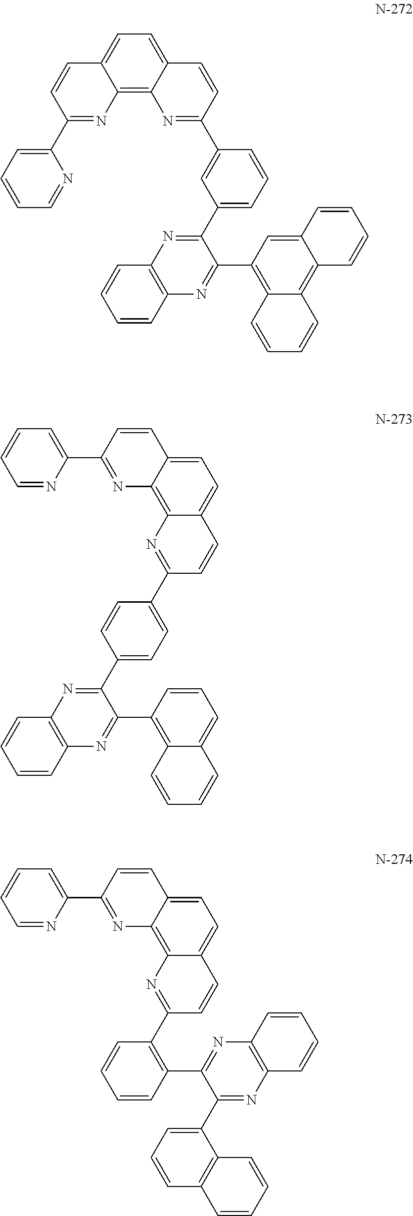 [SMILES: c1ccc(-c2ccc3ccc4ccc(-c5cccc(-c6nc7ccccc7nc6-c6cc7ccccc7c7ccccc67)c5)nc4c3n2)nc1 ; c1ccc(-c2ccc3ccc4ccc(-c5ccc(-c6nc7ccccc7nc6-c6cccc7ccccc67)cc5)nc4c3n2)nc1 ; c1ccc(-c2ccc3ccc4ccc(-c5ccccc5-c5nc6ccccc6nc5-c5cccc6ccccc56)nc4c3n2)nc1]

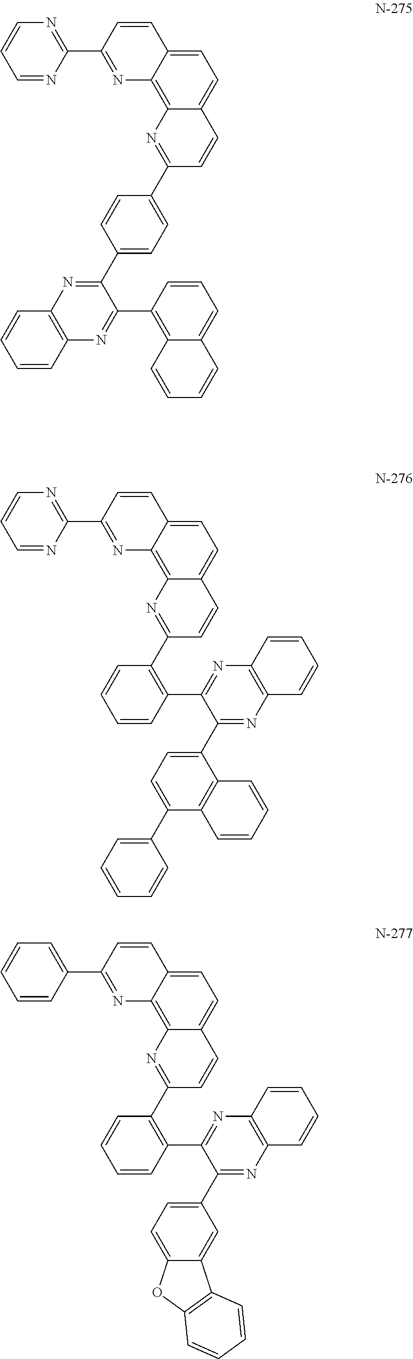

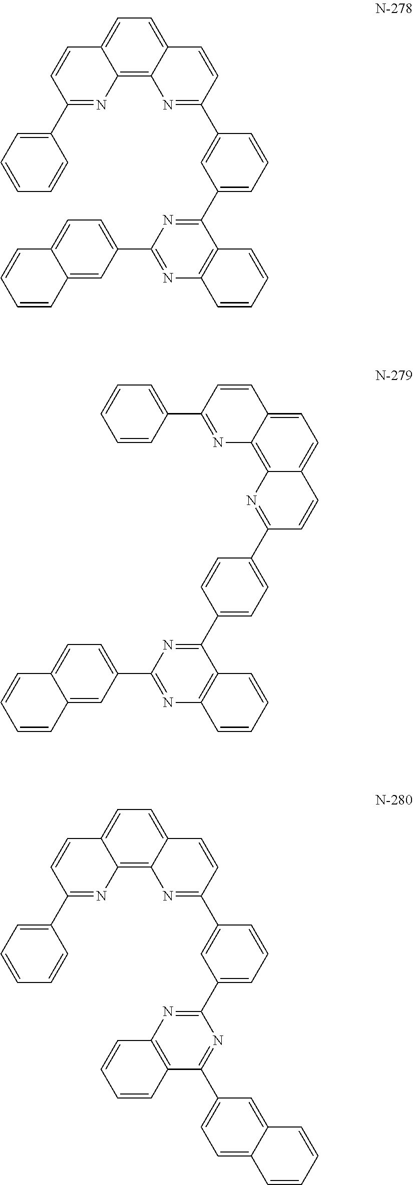 [SMILES: c1ccc(-c2ccc3ccc4ccc(-c5cccc(-c6nc(-c7ccc8ccccc8c7)nc7ccccc67)c5)nc4c3n2)cc1 ; c1ccc(-c2ccc3ccc4ccc(-c5ccc(-c6nc(-c7ccc8ccccc8c7)nc7ccccc67)cc5)nc4c3n2)cc1 ; c1ccc(-c2ccc3ccc4ccc(-c5cccc(-c6nc(-c7ccc8ccccc8c7)c7ccccc7n6)c5)nc4c3n2)cc1]

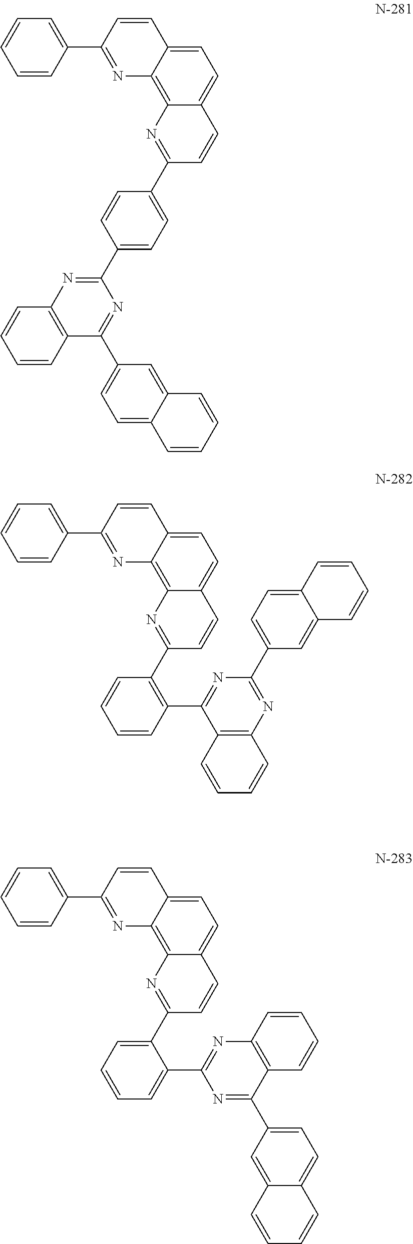 [SMILES: c1ccc(-c2ccc3ccc4ccc(-c5ccc(-c6nc(-c7ccc8ccccc8c7)c7ccccc7n6)cc5)nc4c3n2)cc1 ; c1ccc(-c2ccc3ccc4ccc(-c5ccccc5-c5nc(-c6ccc7ccccc7c6)nc6ccccc56)nc4c3n2)cc1 ; c1ccc(-c2ccc3ccc4ccc(-c5ccccc5-c5nc(-c6ccc7ccccc7c6)c6ccccc6n5)nc4c3n2)cc1]

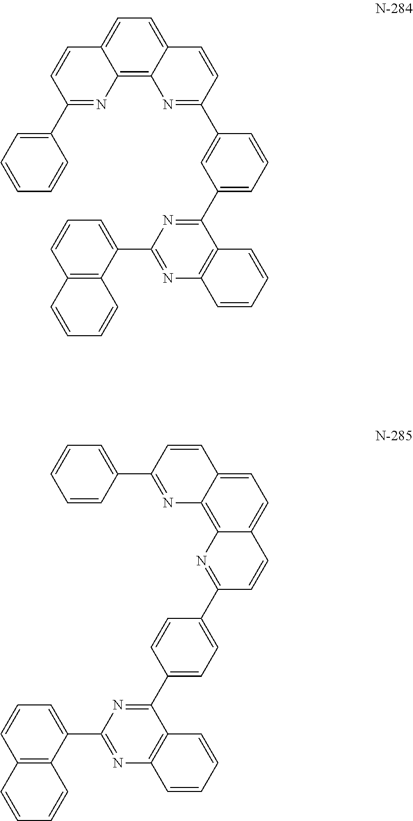 [SMILES: c1ccc(-c2ccc3ccc4ccc(-c5cccc(-c6nc(-c7cccc8ccccc78)nc7ccccc67)c5)nc4c3n2)cc1 ; c1ccc(-c2ccc3ccc4ccc(-c5ccc(-c6nc(-c7cccc8ccccc78)nc7ccccc67)cc5)nc4c3n2)cc1]

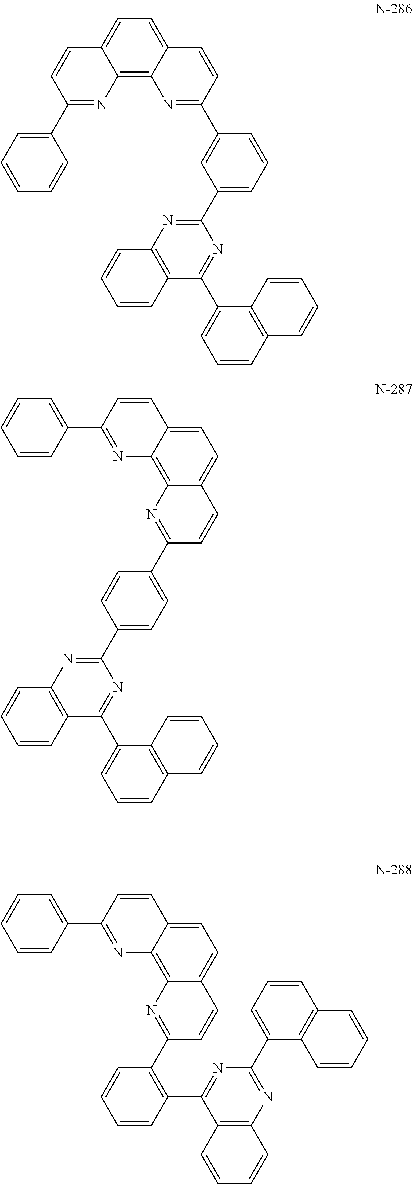 [SMILES: c1ccc(-c2ccc3ccc4ccc(-c5cccc(-c6nc(-c7cccc8ccccc78)c7ccccc7n6)c5)nc4c3n2)cc1 ; c1ccc(-c2ccc3ccc4ccc(-c5ccc(-c6nc(-c7cccc8ccccc78)c7ccccc7n6)cc5)nc4c3n2)cc1 ; c1ccc(-c2ccc3ccc4ccc(-c5ccccc5-c5nc(-c6cccc7ccccc67)nc6ccccc56)nc4c3n2)cc1]

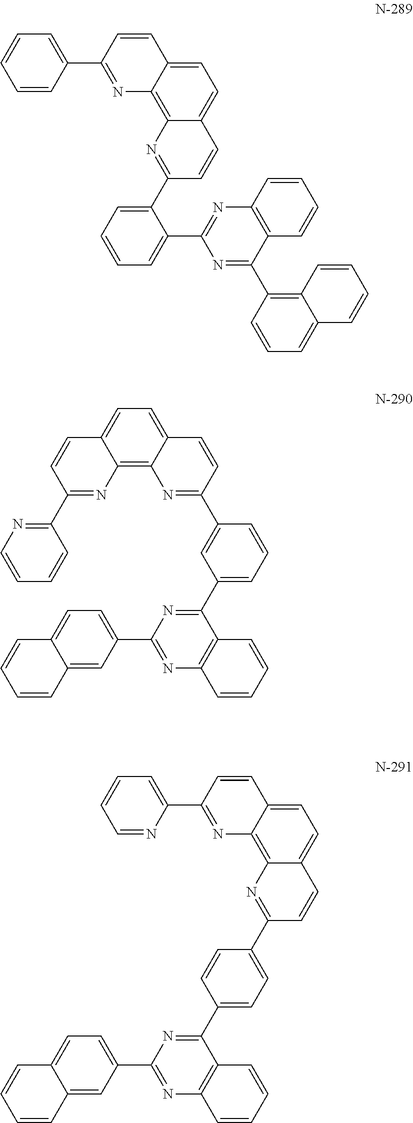 [SMILES: c1ccc(-c2ccc3ccc4ccc(-c5ccccc5-c5nc(-c6cccc7ccccc67)c6ccccc6n5)nc4c3n2)cc1 ; c1ccc(-c2ccc3ccc4ccc(-c5cccc(-c6nc(-c7ccc8ccccc8c7)nc7ccccc67)c5)nc4c3n2)nc1 ; c1ccc(-c2ccc3ccc4ccc(-c5ccc(-c6nc(-c7ccc8ccccc8c7)nc7ccccc67)cc5)nc4c3n2)nc1]

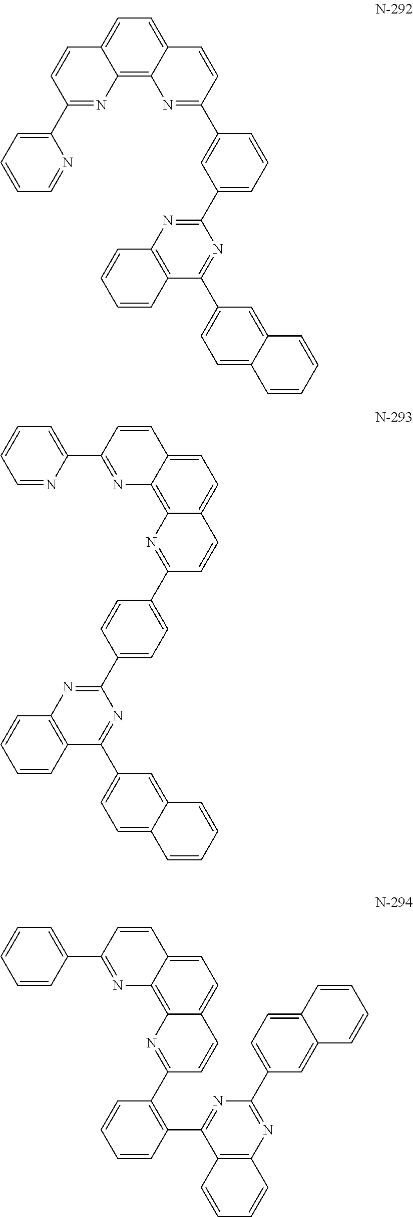 [SMILES: c1ccc(-c2ccc3ccc4ccc(-c5cccc(-c6nc(-c7ccc8ccccc8c7)c7ccccc7n6)c5)nc4c3n2)nc1 ; c1ccc(-c2ccc3ccc4ccc(-c5ccc(-c6nc(-c7ccc8ccccc8c7)c7ccccc7n6)cc5)nc4c3n2)nc1 ; c1ccc(-c2ccc3ccc4ccc(-c5ccccc5-c5nc(-c6ccc7ccccc7c6)nc6ccccc56)nc4c3n2)cc1]

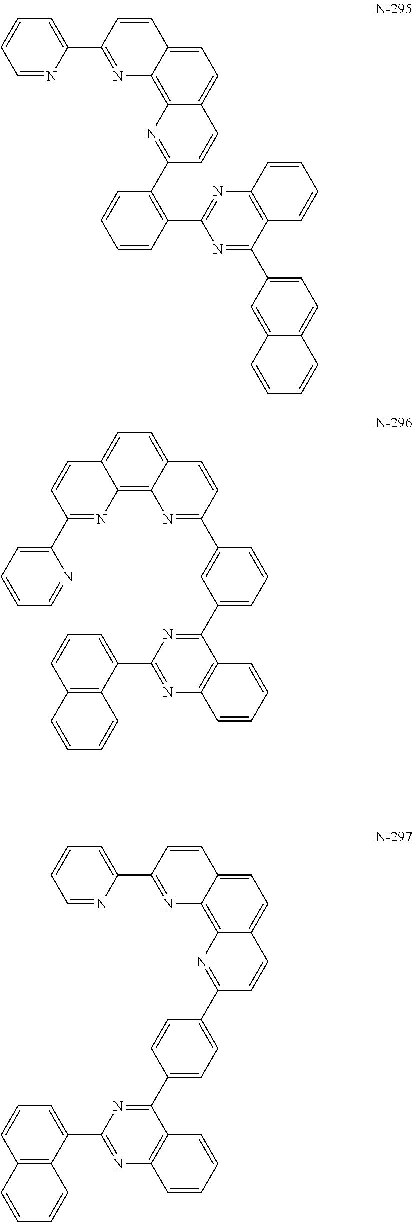 [SMILES: c1ccc(-c2ccc3ccc4ccc(-c5ccccc5-c5nc(-c6ccc7ccccc7c6)c6ccccc6n5)nc4c3n2)nc1 ; c1ccc(-c2ccc3ccc4ccc(-c5cccc(-c6nc(-c7cccc8ccccc78)nc7ccccc67)c5)nc4c3n2)nc1 ; c1ccc(-c2ccc3ccc4ccc(-c5ccc(-c6nc(-c7cccc8ccccc78)nc7ccccc67)cc5)nc4c3n2)nc1]

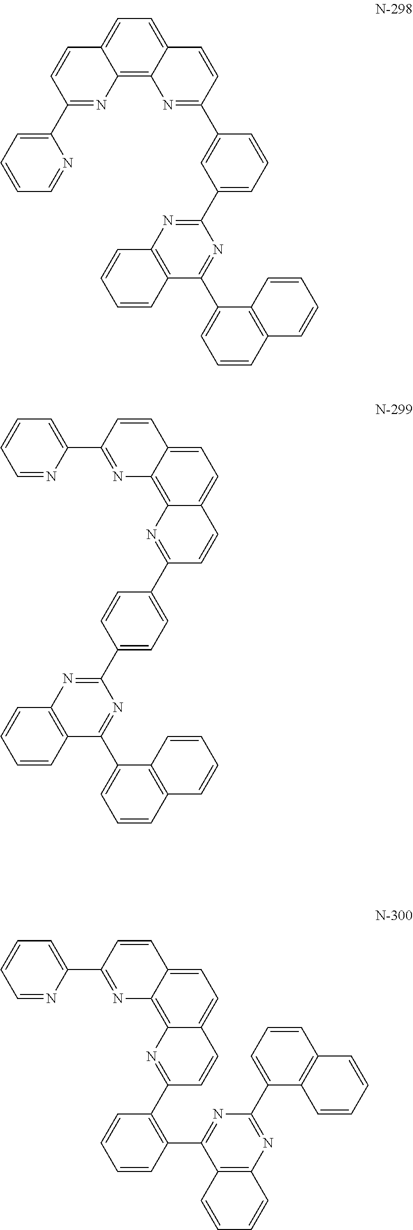 [SMILES: c1ccc(-c2ccc3ccc4ccc(-c5cccc(-c6nc(-c7cccc8ccccc78)c7ccccc7n6)c5)nc4c3n2)nc1 ; c1ccc(-c2ccc3ccc4ccc(-c5ccc(-c6nc(-c7cccc8ccccc78)c7ccccc7n6)cc5)nc4c3n2)nc1 ; c1ccc(-c2ccc3ccc4ccc(-c5ccccc5-c5nc(-c6cccc7ccccc67)nc6ccccc56)nc4c3n2)nc1]

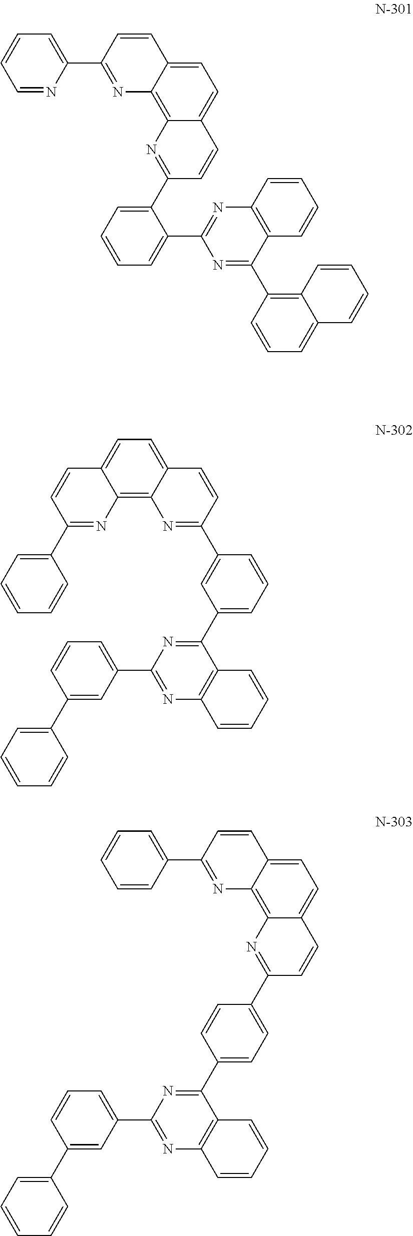 [SMILES: c1ccc(-c2ccc3ccc4ccc(-c5ccccc5-c5nc(-c6cccc7ccccc67)c6ccccc6n5)nc4c3n2)nc1 ; c1ccc(-c2cccc(-c3nc(-c4cccc(-c5ccc6ccc7ccc(-c8ccccc8)nc7c6n5)c4)c4ccccc4n3)c2)cc1 ; c1ccc(-c2cccc(-c3nc(-c4ccc(-c5ccc6ccc7ccc(-c8ccccc8)nc7c6n5)cc4)c4ccccc4n3)c2)cc1]

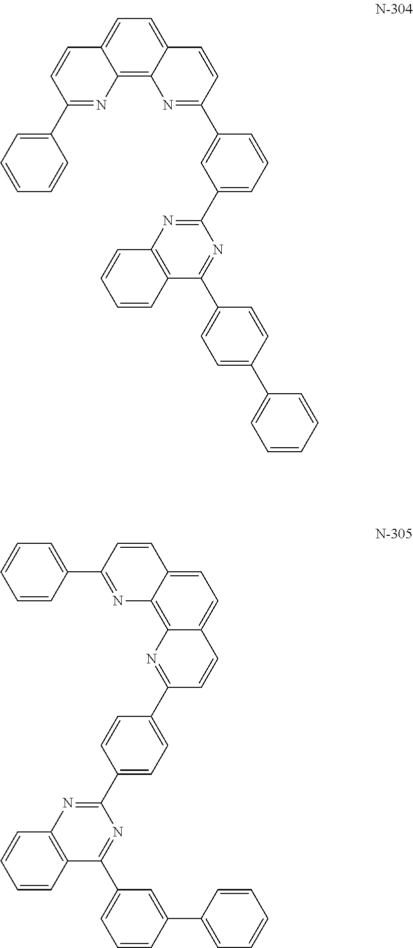 [SMILES: c1ccc(-c2ccc(-c3nc(-c4cccc(-c5ccc6ccc7ccc(-c8ccccc8)nc7c6n5)c4)nc4ccccc34)cc2)cc1 ; c1ccc(-c2cccc(-c3nc(-c4ccc(-c5ccc6ccc7ccc(-c8ccccc8)nc7c6n5)cc4)nc4ccccc34)c2)cc1]

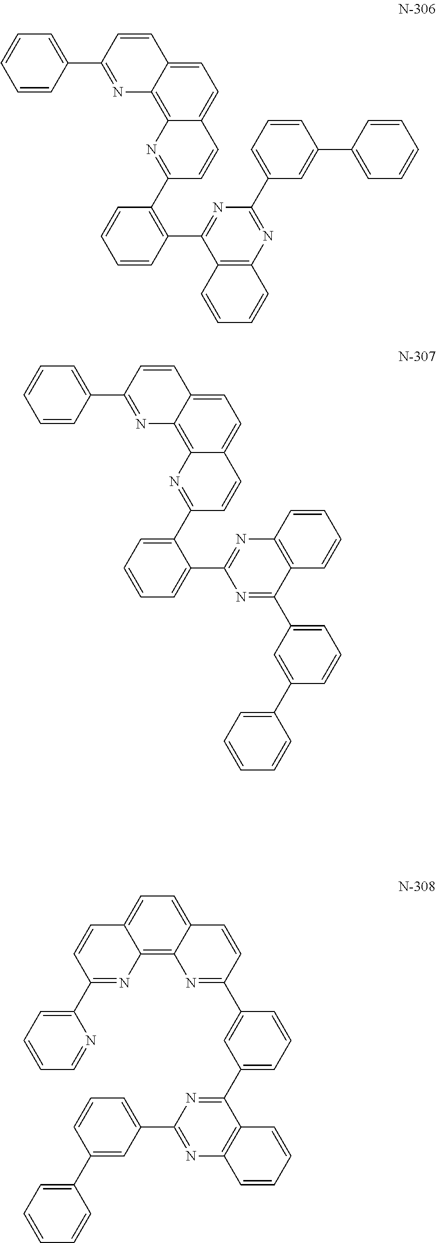 [SMILES: c1ccc(-c2cccc(-c3nc(-c4ccccc4-c4ccc5ccc6ccc(-c7ccccc7)nc6c5n4)c4ccccc4n3)c2)cc1 ; c1ccc(-c2cccc(-c3nc(-c4ccccc4-c4ccc5ccc6ccc(-c7ccccc7)nc6c5n4)nc4ccccc34)c2)cc1 ; c1ccc(-c2cccc(-c3nc(-c4cccc(-c5ccc6ccc7ccc(-c8ccccn8)nc7c6n5)c4)c4ccccc4n3)c2)cc1]

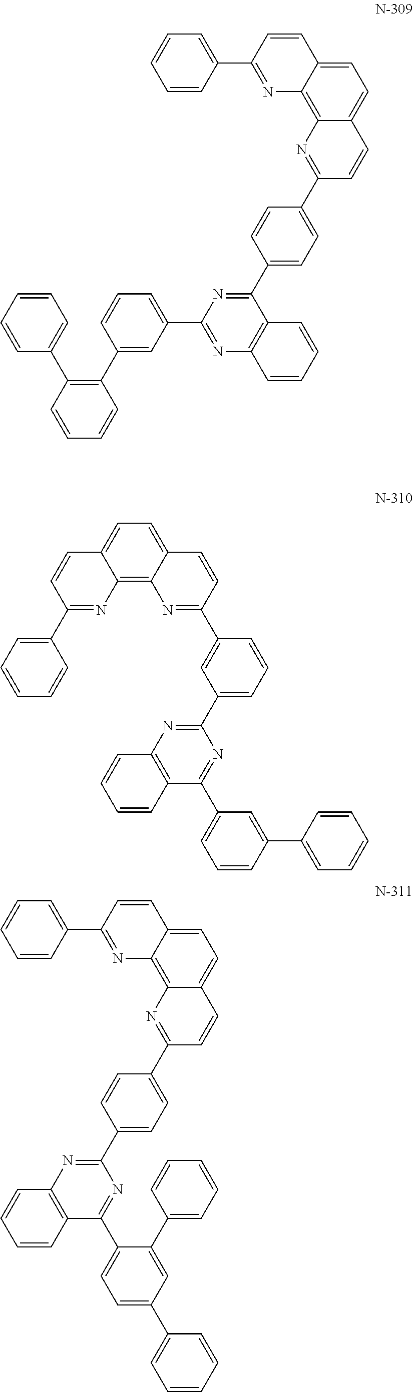 [SMILES: c1ccc(-c2ccc3ccc4ccc(-c5ccc(-c6nc(-c7cccc(-c8ccccc8-c8ccccc8)c7)nc7ccccc67)cc5)nc4c3n2)cc1 ; c1ccc(-c2cccc(-c3nc(-c4cccc(-c5ccc6ccc7ccc(-c8ccccc8)nc7c6n5)c4)nc4ccccc34)c2)cc1 ; c1ccc(-c2ccc(-c3nc(-c4ccc(-c5ccc6ccc7ccc(-c8ccccc8)nc7c6n5)cc4)nc4ccccc34)c(-c3ccccc3)c2)cc1]

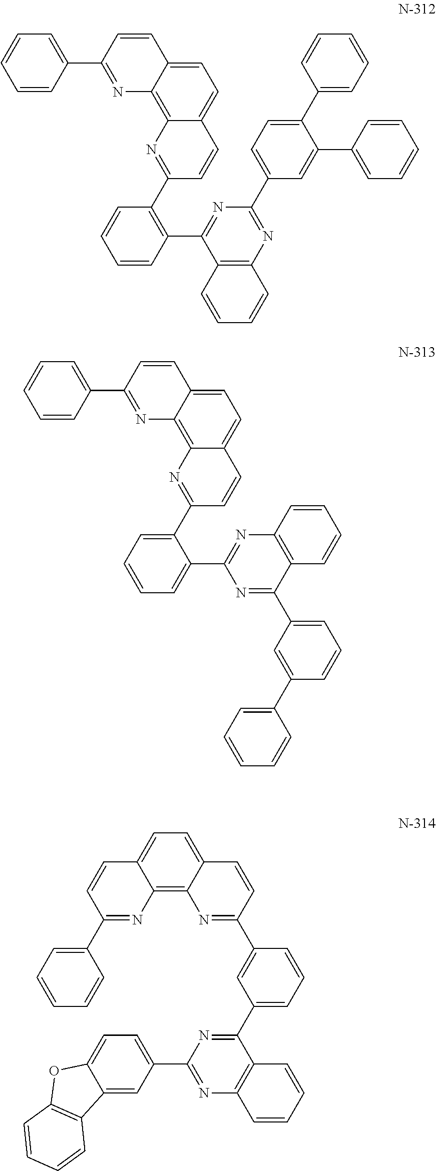 [SMILES: c1ccc(-c2ccc3ccc4ccc(-c5ccccc5-c5nc(-c6ccc(-c7ccccc7)c(-c7ccccc7)c6)nc6ccccc56)nc4c3n2)cc1 ; c1ccc(-c2cccc(-c3nc(-c4ccccc4-c4ccc5ccc6ccc(-c7ccccc7)nc6c5n4)nc4ccccc34)c2)cc1 ; c1ccc(-c2ccc3ccc4ccc(-c5cccc(-c6nc(-c7ccc8oc9ccccc9c8c7)nc7ccccc67)c5)nc4c3n2)cc1]

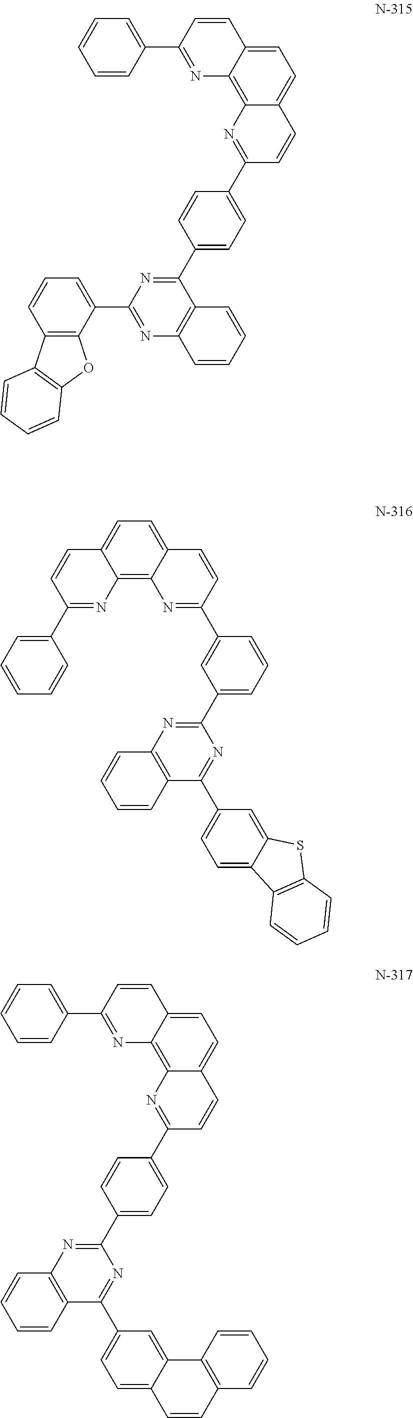 [SMILES: c1ccc(-c2ccc3ccc4ccc(-c5ccc(-c6nc(-c7cccc8c7oc7ccccc78)nc7ccccc67)cc5)nc4c3n2)cc1 ; c1ccc(-c2ccc3ccc4ccc(-c5cccc(-c6nc(-c7ccc8c(c7)sc7ccccc78)c7ccccc7n6)c5)nc4c3n2)cc1 ; c1ccc(-c2ccc3ccc4ccc(-c5ccc(-c6nc(-c7ccc8ccc9ccccc9c8c7)c7ccccc7n6)cc5)nc4c3n2)cc1]

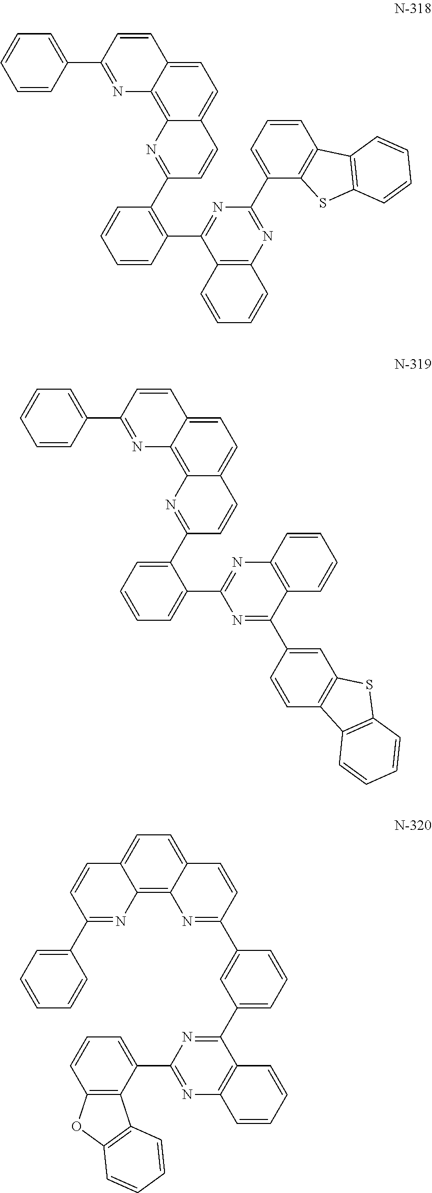 [SMILES: c1ccc(-c2ccc3ccc4ccc(-c5ccccc5-c5nc(-c6cccc7c6sc6ccccc67)nc6ccccc56)nc4c3n2)cc1 ; c1ccc(-c2ccc3ccc4ccc(-c5ccccc5-c5nc(-c6ccc7c(c6)sc6ccccc67)c6ccccc6n5)nc4c3n2)cc1 ; c1ccc(-c2ccc3ccc4ccc(-c5cccc(-c6nc(-c7cccc8oc9ccccc9c78)nc7ccccc67)c5)nc4c3n2)cc1]

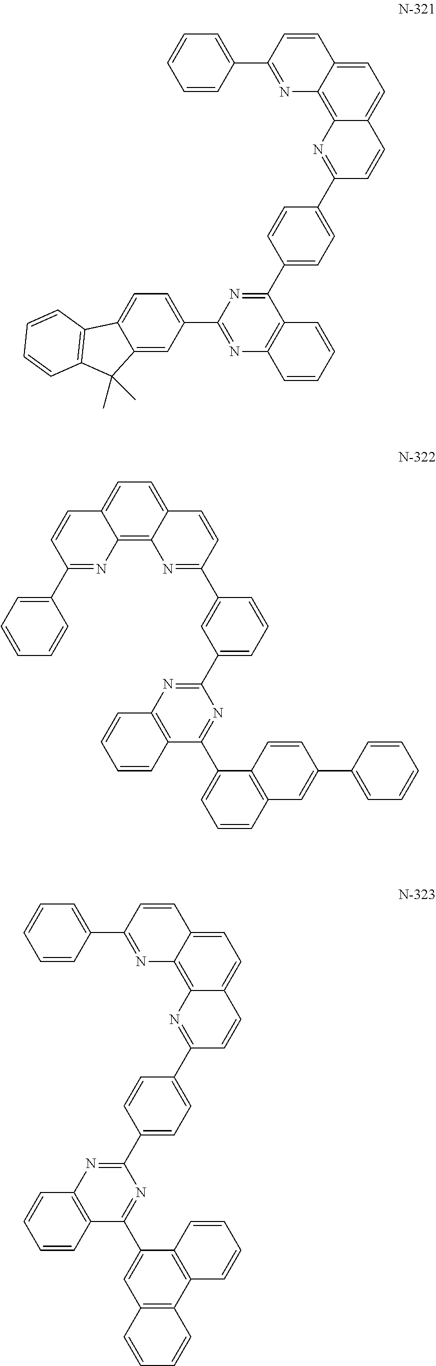 [SMILES: CC1(C)c2ccccc2-c2ccc(-c3nc(-c4ccc(-c5ccc6ccc7ccc(-c8ccccc8)nc7c6n5)cc4)c4ccccc4n3)cc21 ; c1ccc(-c2ccc3c(-c4nc(-c5cccc(-c6ccc7ccc8ccc(-c9ccccc9)nc8c7n6)c5)nc5ccccc45)cccc3c2)cc1 ; c1ccc(-c2ccc3ccc4ccc(-c5ccc(-c6nc(-c7cc8ccccc8c8ccccc78)c7ccccc7n6)cc5)nc4c3n2)cc1]

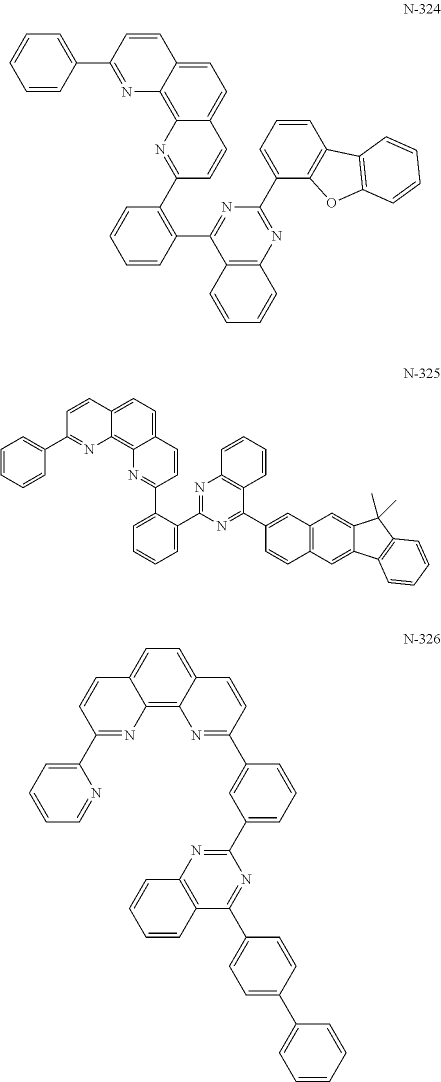 [SMILES: c1ccc(-c2ccc3ccc4ccc(-c5ccccc5-c5nc(-c6cccc7c6oc6ccccc67)nc6ccccc56)nc4c3n2)cc1 ; CC1(C)c2ccccc2-c2cc3ccc(-c4nc(-c5ccccc5-c5ccc6ccc7ccc(-c8ccccc8)nc7c6n5)nc5ccccc45)cc3cc21 ; c1ccc(-c2ccc(-c3nc(-c4cccc(-c5ccc6ccc7ccc(-c8ccccn8)nc7c6n5)c4)nc4ccccc34)cc2)cc1]

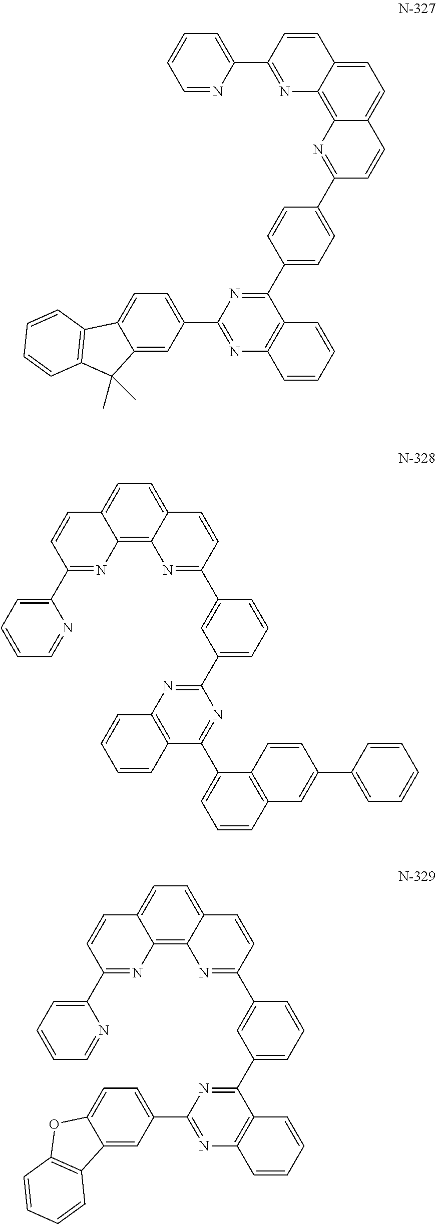 [SMILES: CC1(C)c2ccccc2-c2ccc(-c3nc(-c4ccc(-c5ccc6ccc7ccc(-c8ccccn8)nc7c6n5)cc4)c4ccccc4n3)cc21 ; c1ccc(-c2ccc3c(-c4nc(-c5cccc(-c6ccc7ccc8ccc(-c9ccccn9)nc8c7n6)c5)nc5ccccc45)cccc3c2)cc1 ; c1ccc(-c2ccc3ccc4ccc(-c5cccc(-c6nc(-c7ccc8oc9ccccc9c8c7)nc7ccccc67)c5)nc4c3n2)nc1]

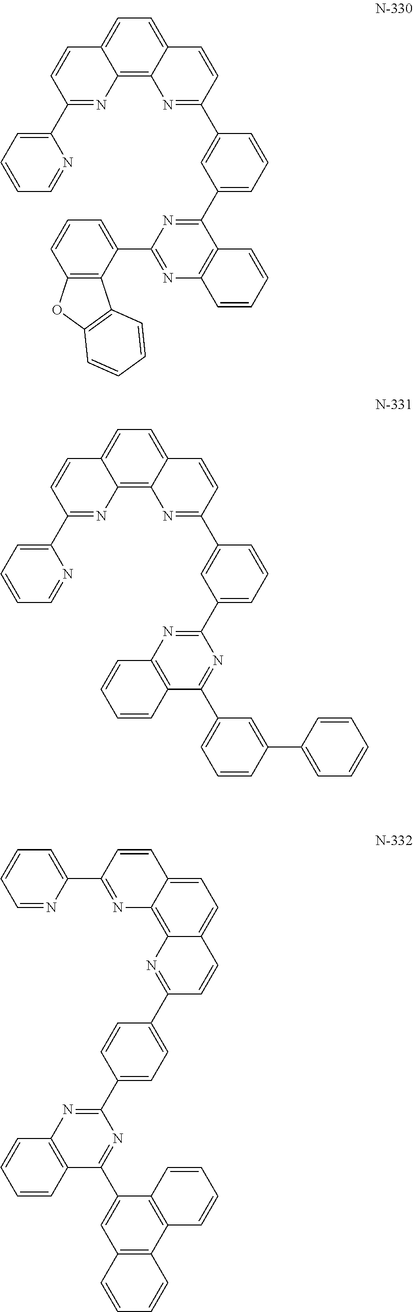 [SMILES: c1ccc(-c2ccc3ccc4ccc(-c5cccc(-c6nc(-c7cccc8oc9ccccc9c78)nc7ccccc67)c5)nc4c3n2)nc1 ; c1ccc(-c2cccc(-c3nc(-c4cccc(-c5ccc6ccc7ccc(-c8ccccn8)nc7c6n5)c4)nc4ccccc34)c2)cc1 ; c1ccc(-c2ccc3ccc4ccc(-c5ccc(-c6nc(-c7cc8ccccc8c8ccccc78)c7ccccc7n6)cc5)nc4c3n2)nc1]

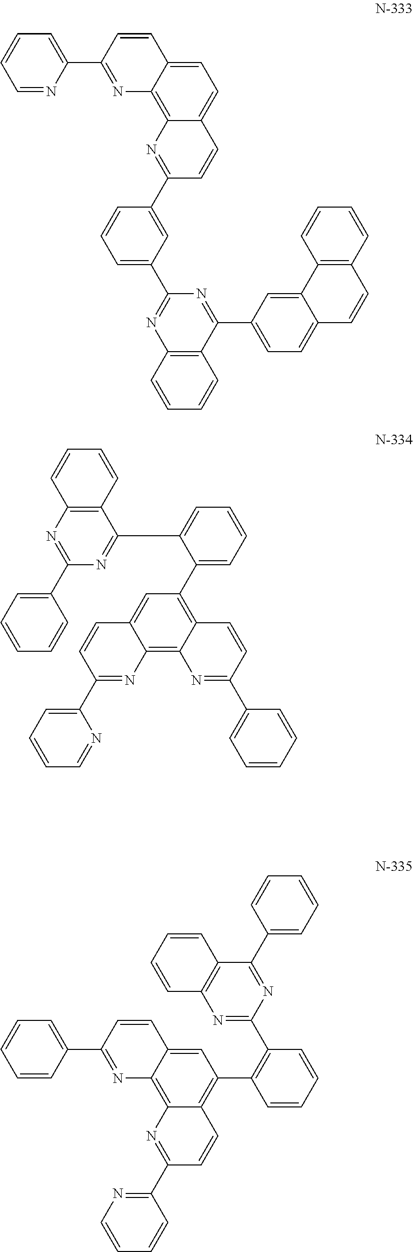 [SMILES: c1ccc(-c2ccc3ccc4ccc(-c5cccc(-c6nc(-c7ccc8ccc9ccccc9c8c7)c7ccccc7n6)c5)nc4c3n2)nc1 ; c1ccc(-c2ccc3c(-c4ccccc4-c4nc(-c5ccccc5)nc5ccccc45)cc4ccc(-c5ccccn5)nc4c3n2)cc1 ; c1ccc(-c2ccc3cc(-c4ccccc4-c4nc(-c5ccccc5)c5ccccc5n4)c4ccc(-c5ccccn5)nc4c3n2)cc1]

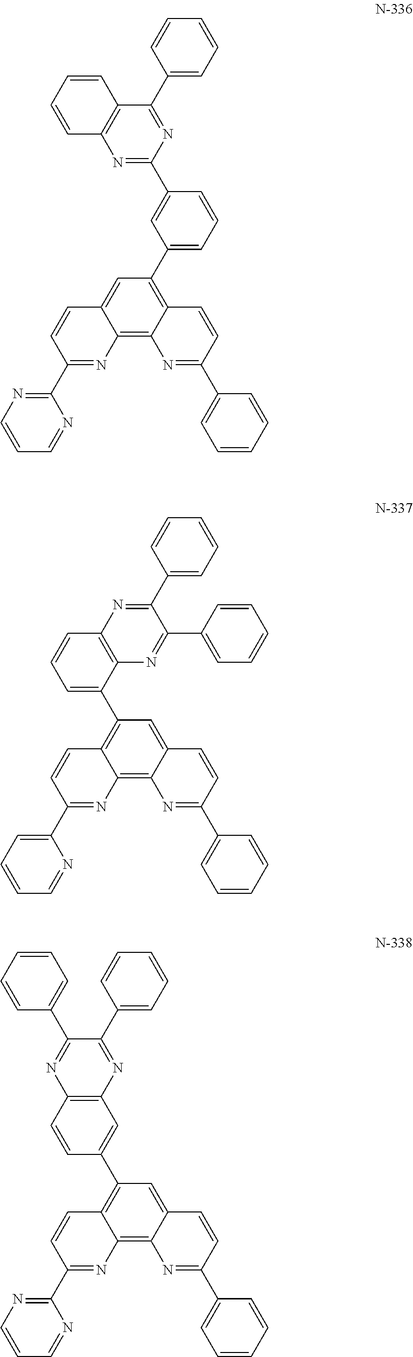 [SMILES: c1ccc(-c2ccc3c(-c4cccc(-c5nc(-c6ccccc6)c6ccccc6n5)c4)cc4ccc(-c5ncccn5)nc4c3n2)cc1 ; c1ccc(-c2ccc3cc(-c4cccc5nc(-c6ccccc6)c(-c6ccccc6)nc45)c4ccc(-c5ccccn5)nc4c3n2)cc1 ; c1ccc(-c2ccc3cc(-c4ccc5nc(-c6ccccc6)c(-c6ccccc6)nc5c4)c4ccc(-c5ncccn5)nc4c3n2)cc1]

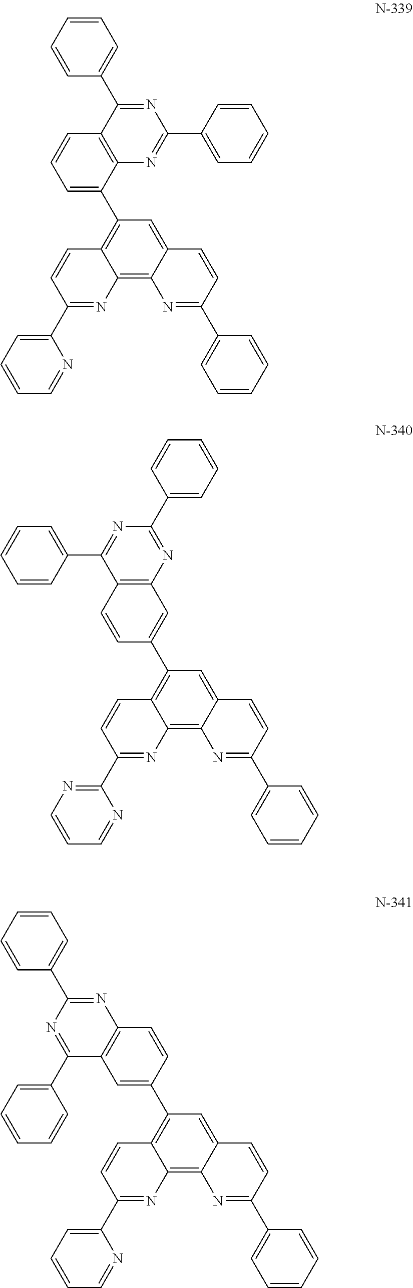 [SMILES: c1ccc(-c2ccc3cc(-c4cccc5c(-c6ccccc6)nc(-c6ccccc6)nc45)c4ccc(-c5ccccn5)nc4c3n2)cc1 ; c1ccc(-c2ccc3cc(-c4ccc5c(-c6ccccc6)nc(-c6ccccc6)nc5c4)c4ccc(-c5ncccn5)nc4c3n2)cc1 ; c1ccc(-c2ccc3cc(-c4ccc5nc(-c6ccccc6)nc(-c6ccccc6)c5c4)c4ccc(-c5ccccn5)nc4c3n2)cc1]

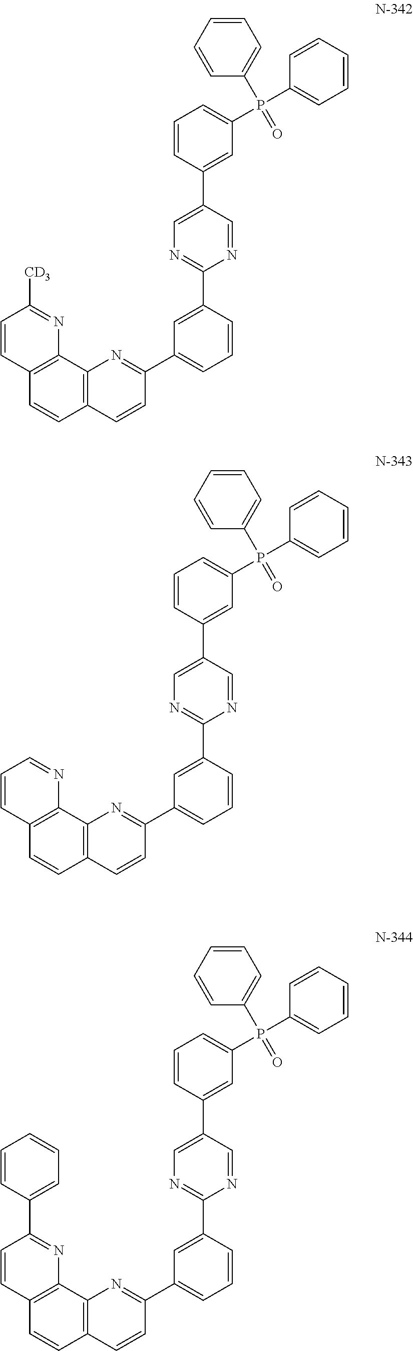 [SMILES: [2H]C([2H])([2H])c1ccc2ccc3ccc(-c4cccc(-c5ncc(-c6cccc(P(=O)(c7ccccc7)c7ccccc7)c6)cn5)c4)nc3c2n1 ; O=P(c1ccccc1)(c1ccccc1)c1cccc(-c2cnc(-c3cccc(-c4ccc5ccc6cccnc6c5n4)c3)nc2)c1 ; O=P(c1ccccc1)(c1ccccc1)c1cccc(-c2cnc(-c3cccc(-c4ccc5ccc6ccc(-c7ccccc7)nc6c5n4)c3)nc2)c1]

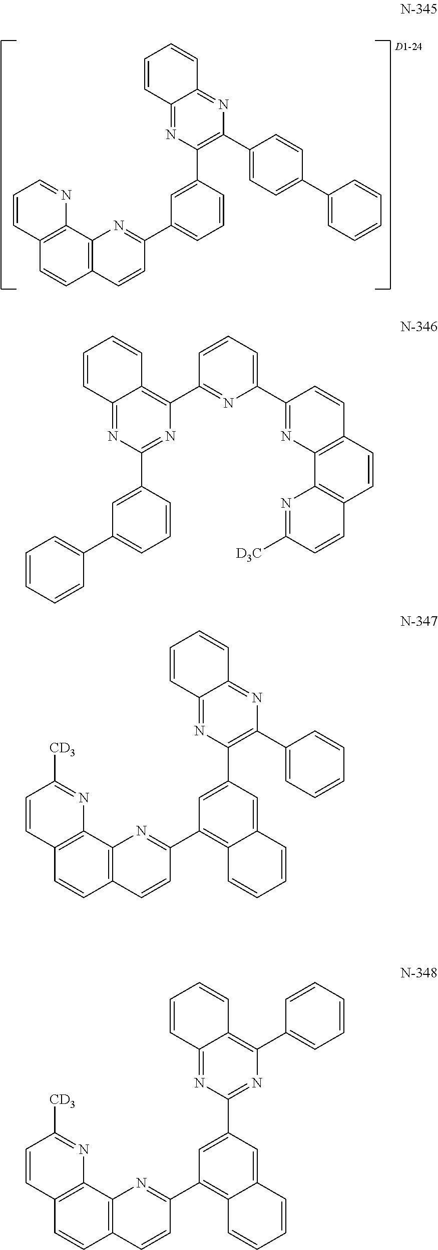

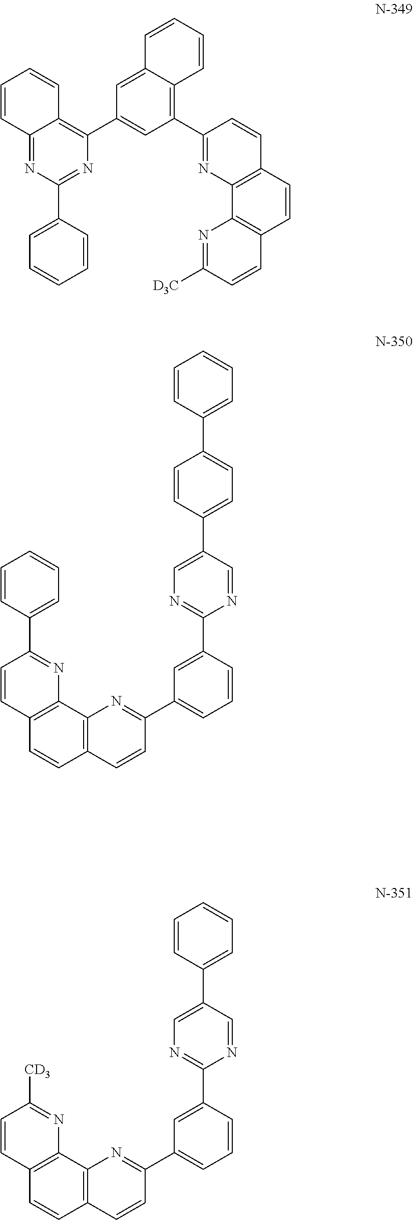 [SMILES: [2H]C([2H])([2H])c1ccc2ccc3ccc(-c4cc(-c5nc(-c6ccccc6)nc6ccccc56)cc5ccccc45)nc3c2n1 ; c1ccc(-c2ccc(-c3cnc(-c4cccc(-c5ccc6ccc7ccc(-c8ccccc8)nc7c6n5)c4)nc3)cc2)cc1 ; [2H]C([2H])([2H])c1ccc2ccc3ccc(-c4cccc(-c5ncc(-c6ccccc6)cn5)c4)nc3c2n1]

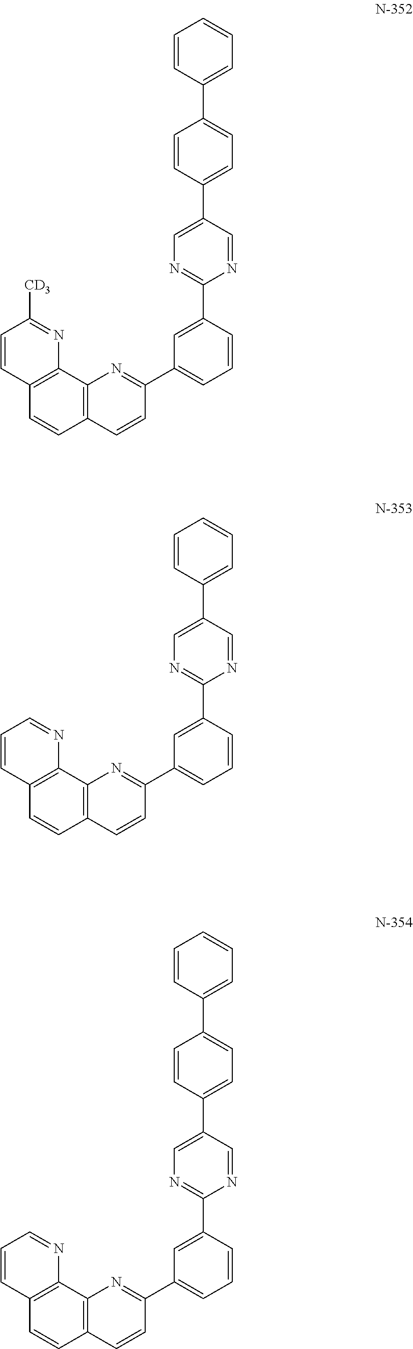 [SMILES: [2H]C([2H])([2H])c1ccc2ccc3ccc(-c4cccc(-c5ncc(-c6ccc(-c7ccccc7)cc6)cn5)c4)nc3c2n1 ; c1ccc(-c2cnc(-c3cccc(-c4ccc5ccc6cccnc6c5n4)c3)nc2)cc1 ; c1ccc(-c2ccc(-c3cnc(-c4cccc(-c5ccc6ccc7cccnc7c6n5)c4)nc3)cc2)cc1]

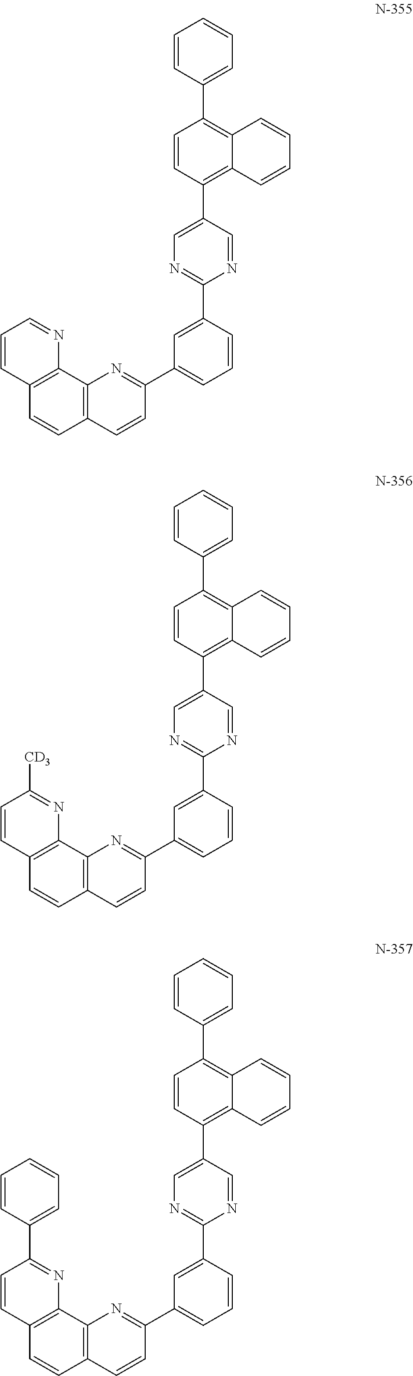 [SMILES: c1ccc(-c2ccc(-c3cnc(-c4cccc(-c5ccc6ccc7cccnc7c6n5)c4)nc3)c3ccccc23)cc1 ; [2H]C([2H])([2H])c1ccc2ccc3ccc(-c4cccc(-c5ncc(-c6ccc(-c7ccccc7)c7ccccc67)cn5)c4)nc3c2n1 ; c1ccc(-c2ccc3ccc4ccc(-c5cccc(-c6ncc(-c7ccc(-c8ccccc8)c8ccccc78)cn6)c5)nc4c3n2)cc1]

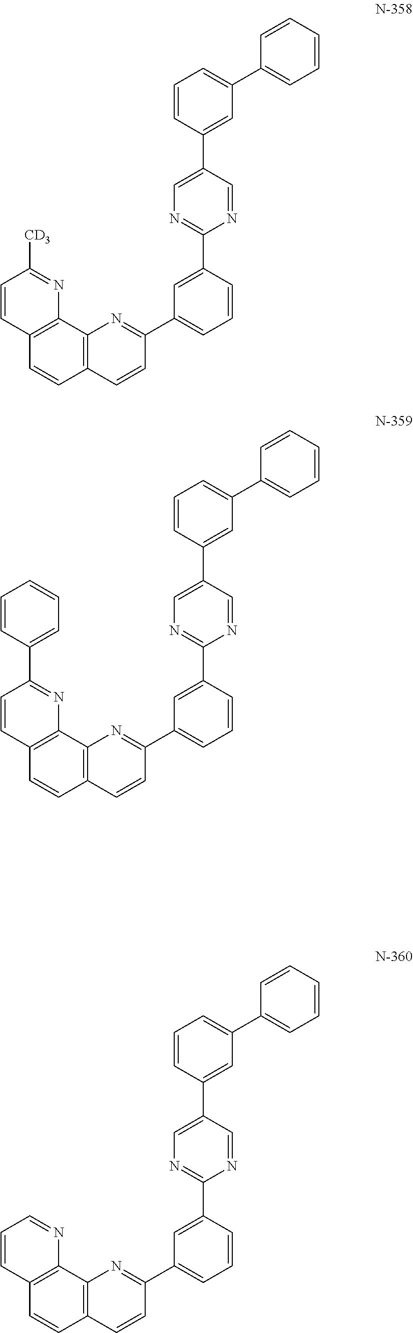 [SMILES: [2H]C([2H])([2H])c1ccc2ccc3ccc(-c4cccc(-c5ncc(-c6cccc(-c7ccccc7)c6)cn5)c4)nc3c2n1 ; c1ccc(-c2cccc(-c3cnc(-c4cccc(-c5ccc6ccc7ccc(-c8ccccc8)nc7c6n5)c4)nc3)c2)cc1 ; c1ccc(-c2cccc(-c3cnc(-c4cccc(-c5ccc6ccc7cccnc7c6n5)c4)nc3)c2)cc1]

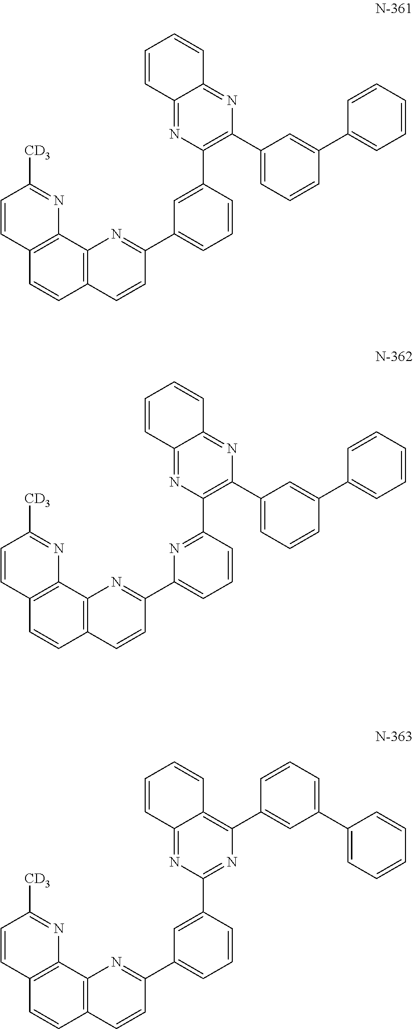 [SMILES: [2H]C([2H])([2H])c1ccc2ccc3ccc(-c4cccc(-c5nc6ccccc6nc5-c5cccc(-c6ccccc6)c5)c4)nc3c2n1 ; [2H]C([2H])([2H])c1ccc2ccc3ccc(-c4cccc(-c5nc6ccccc6nc5-c5cccc(-c6ccccc6)c5)n4)nc3c2n1 ; [2H]C([2H])([2H])c1ccc2ccc3ccc(-c4cccc(-c5nc(-c6cccc(-c7ccccc7)c6)c6ccccc6n5)c4)nc3c2n1]

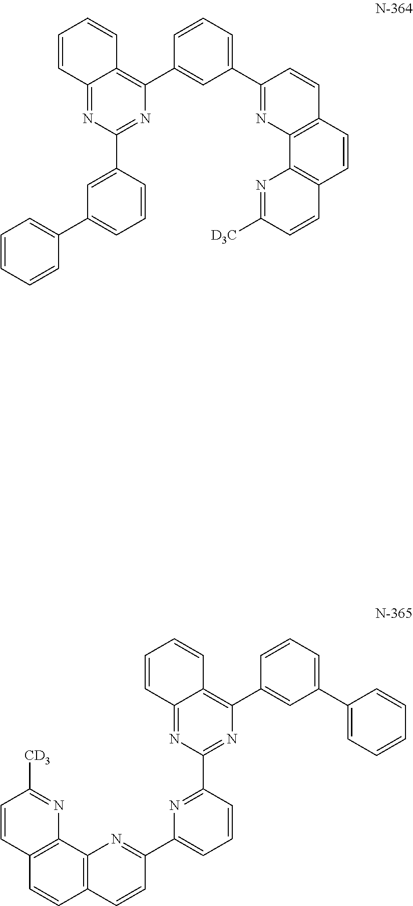 [SMILES: [2H]C([2H])([2H])c1ccc2ccc3ccc(-c4cccc(-c5nc(-c6cccc(-c7ccccc7)c6)nc6ccccc56)c4)nc3c2n1 ; [2H]C([2H])([2H])c1ccc2ccc3ccc(-c4cccc(-c5nc(-c6cccc(-c7ccccc7)c6)c6ccccc6n5)n4)nc3c2n1]

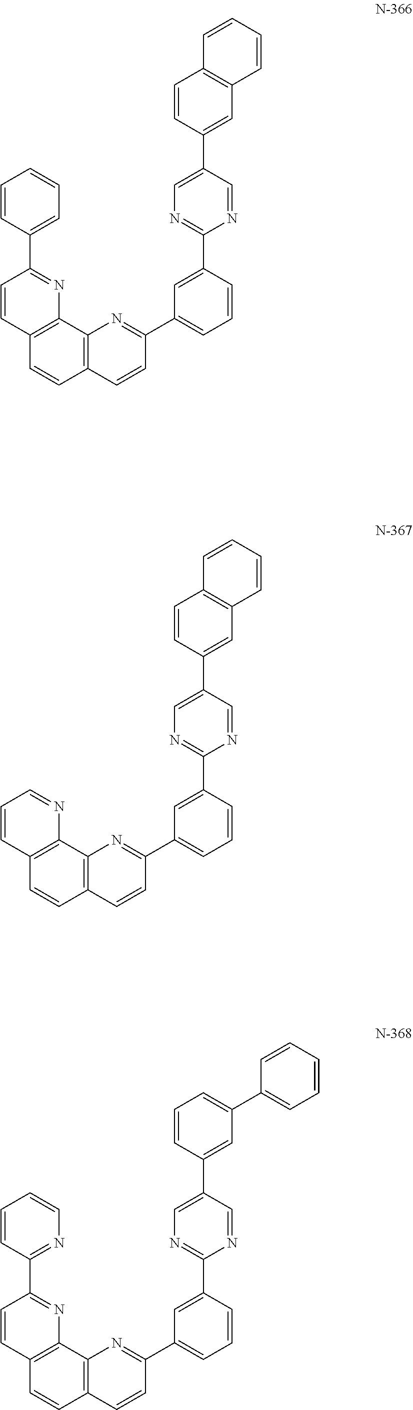 [SMILES: c1ccc(-c2ccc3ccc4ccc(-c5cccc(-c6ncc(-c7ccc8ccccc8c7)cn6)c5)nc4c3n2)cc1 ; c1cc(-c2ccc3ccc4cccnc4c3n2)cc(-c2ncc(-c3ccc4ccccc4c3)cn2)c1 ; c1ccc(-c2cccc(-c3cnc(-c4cccc(-c5ccc6ccc7ccc(-c8ccccn8)nc7c6n5)c4)nc3)c2)cc1]

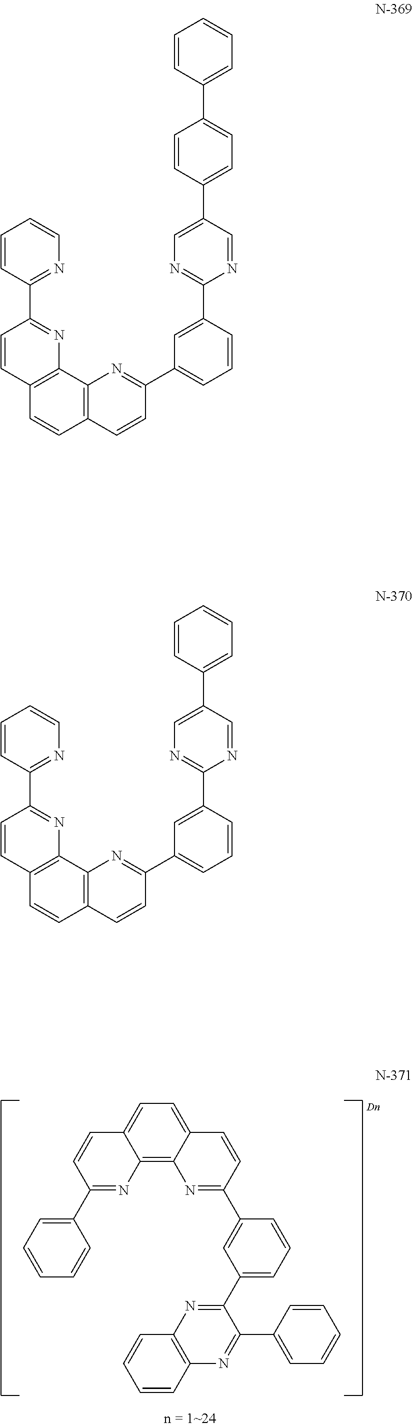

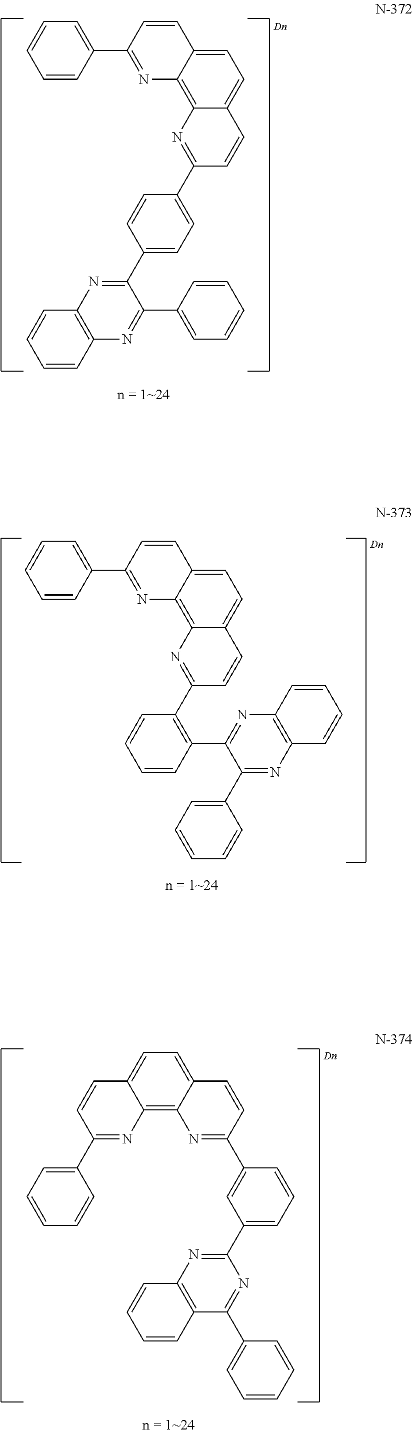

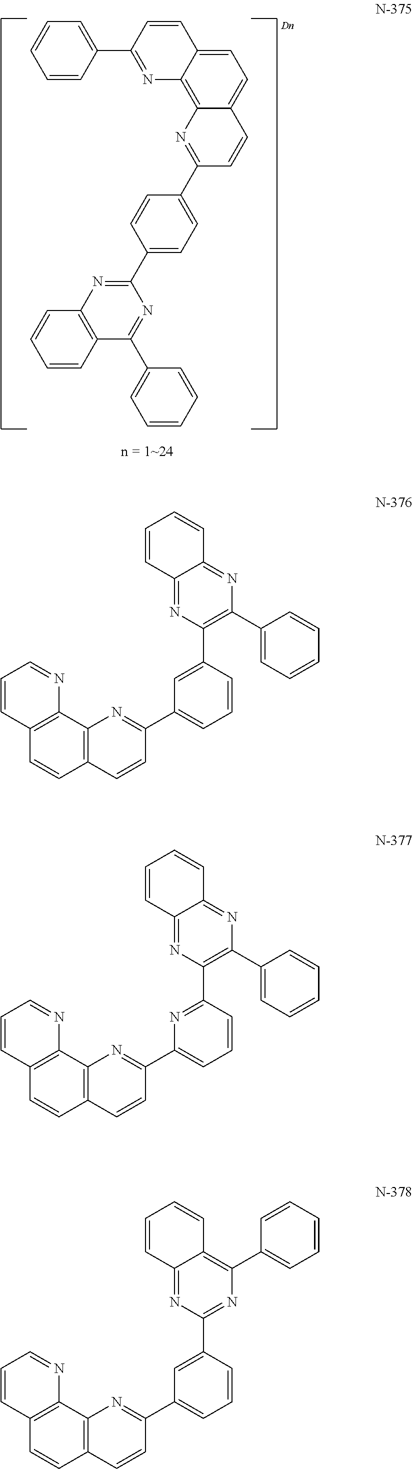

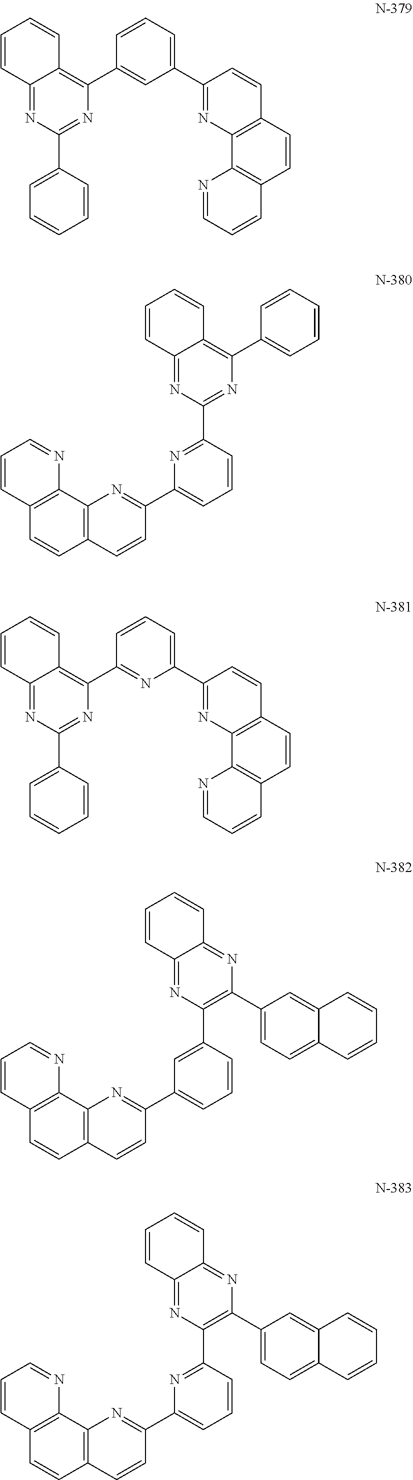 [SMILES: c1ccc(-c2nc(-c3cccc(-c4ccc5ccc6cccnc6c5n4)c3)c3ccccc3n2)cc1 ; c1ccc(-c2nc(-c3cccc(-c4ccc5ccc6cccnc6c5n4)n3)nc3ccccc23)cc1 ; c1ccc(-c2nc(-c3cccc(-c4ccc5ccc6cccnc6c5n4)n3)c3ccccc3n2)cc1 ; c1cc(-c2ccc3ccc4cccnc4c3n2)cc(-c2nc3ccccc3nc2-c2ccc3ccccc3c2)c1 ; c1cc(-c2ccc3ccc4cccnc4c3n2)nc(-c2nc3ccccc3nc2-c2ccc3ccccc3c2)c1]

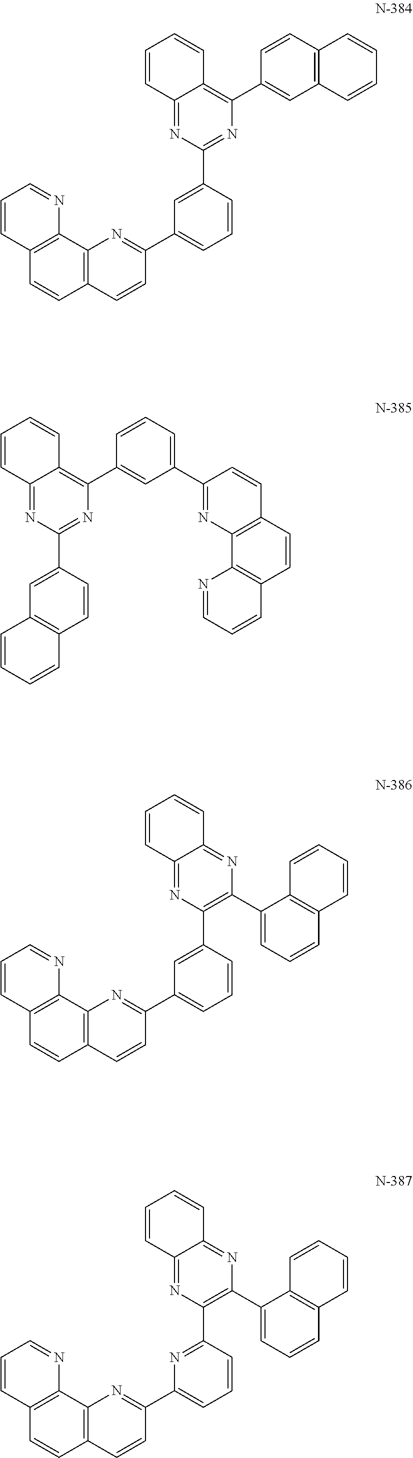 [SMILES: c1cc(-c2ccc3ccc4cccnc4c3n2)cc(-c2nc(-c3ccc4ccccc4c3)c3ccccc3n2)c1 ; c1cc(-c2ccc3ccc4cccnc4c3n2)cc(-c2nc(-c3ccc4ccccc4c3)nc3ccccc23)c1 ; c1cc(-c2ccc3ccc4cccnc4c3n2)cc(-c2nc3ccccc3nc2-c2cccc3ccccc23)c1 ; c1cc(-c2ccc3ccc4cccnc4c3n2)nc(-c2nc3ccccc3nc2-c2cccc3ccccc23)c1]

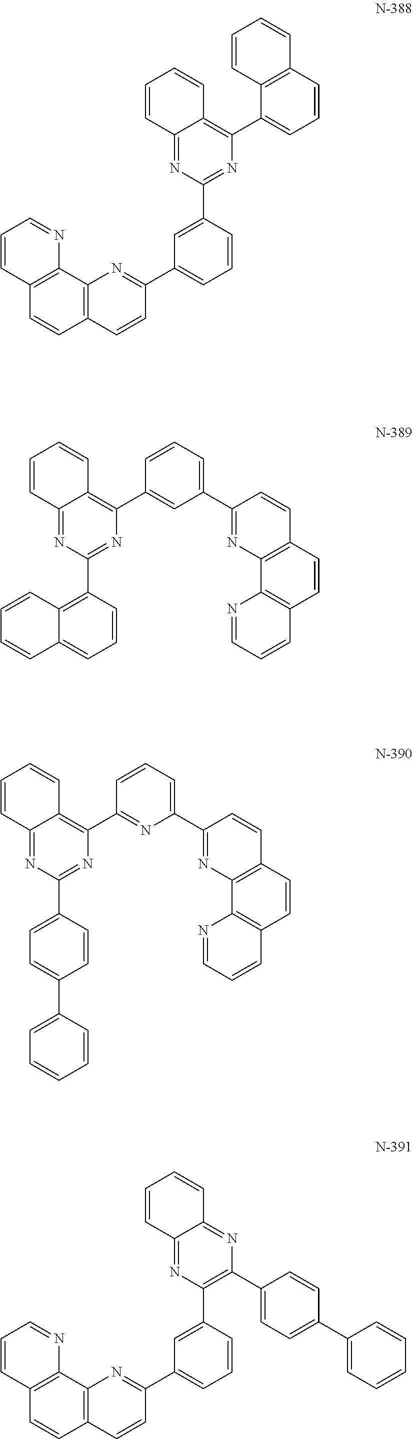

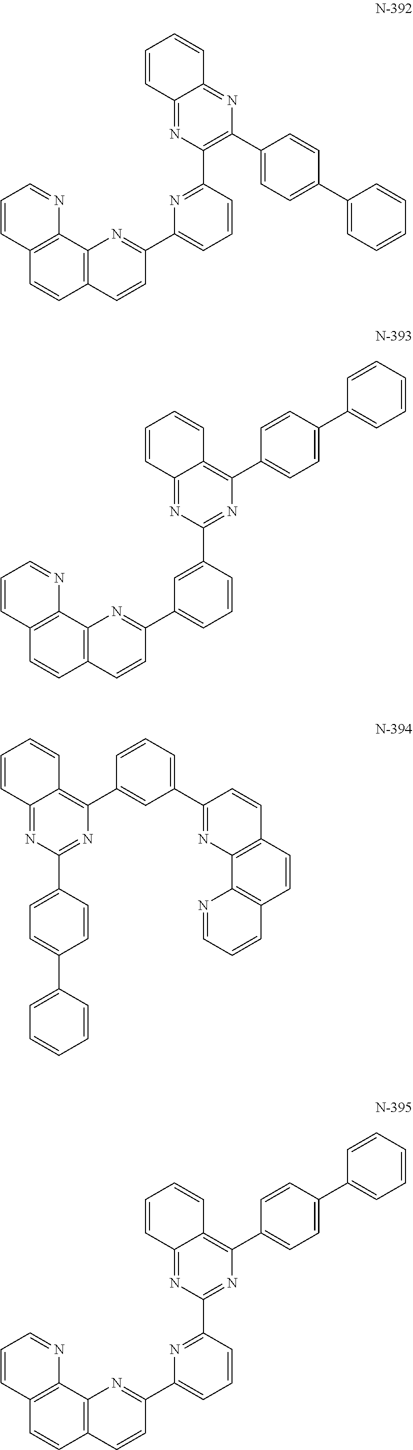 [SMILES: c1ccc(-c2ccc(-c3nc4ccccc4nc3-c3cccc(-c4ccc5ccc6cccnc6c5n4)n3)cc2)cc1 ; c1ccc(-c2ccc(-c3nc(-c4cccc(-c5ccc6ccc7cccnc7c6n5)c4)nc4ccccc34)cc2)cc1 ; c1ccc(-c2ccc(-c3nc(-c4cccc(-c5ccc6ccc7cccnc7c6n5)c4)c4ccccc4n3)cc2)cc1 ; c1ccc(-c2ccc(-c3nc(-c4cccc(-c5ccc6ccc7cccnc7c6n5)n4)nc4ccccc34)cc2)cc1]

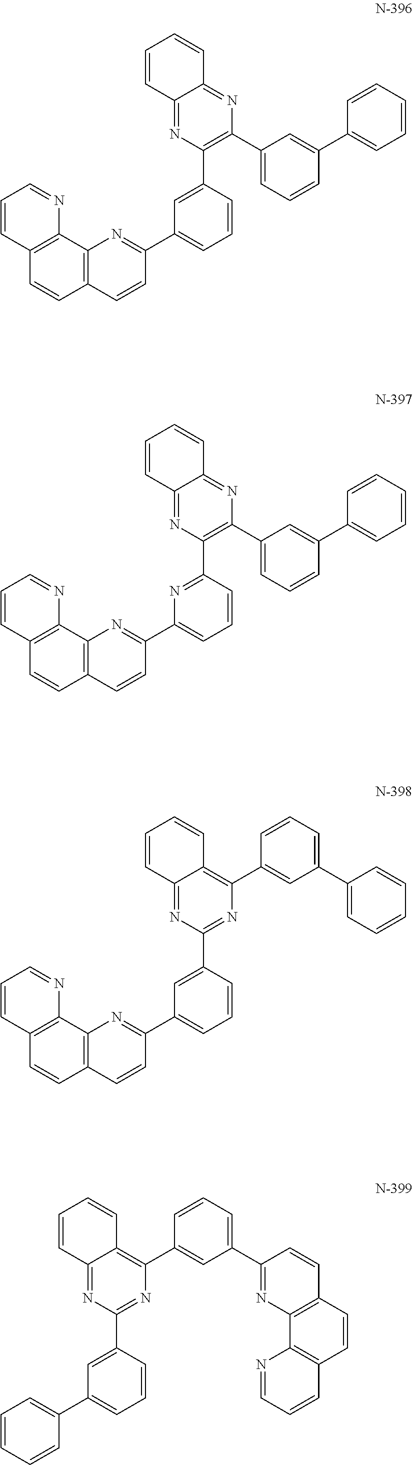 [SMILES: c1ccc(-c2cccc(-c3nc4ccccc4nc3-c3cccc(-c4ccc5ccc6cccnc6c5n4)c3)c2)cc1 ; c1ccc(-c2cccc(-c3nc4ccccc4nc3-c3cccc(-c4ccc5ccc6cccnc6c5n4)n3)c2)cc1 ; c1ccc(-c2cccc(-c3nc(-c4cccc(-c5ccc6ccc7cccnc7c6n5)c4)nc4ccccc34)c2)cc1 ; c1ccc(-c2cccc(-c3nc(-c4cccc(-c5ccc6ccc7cccnc7c6n5)c4)c4ccccc4n3)c2)cc1]

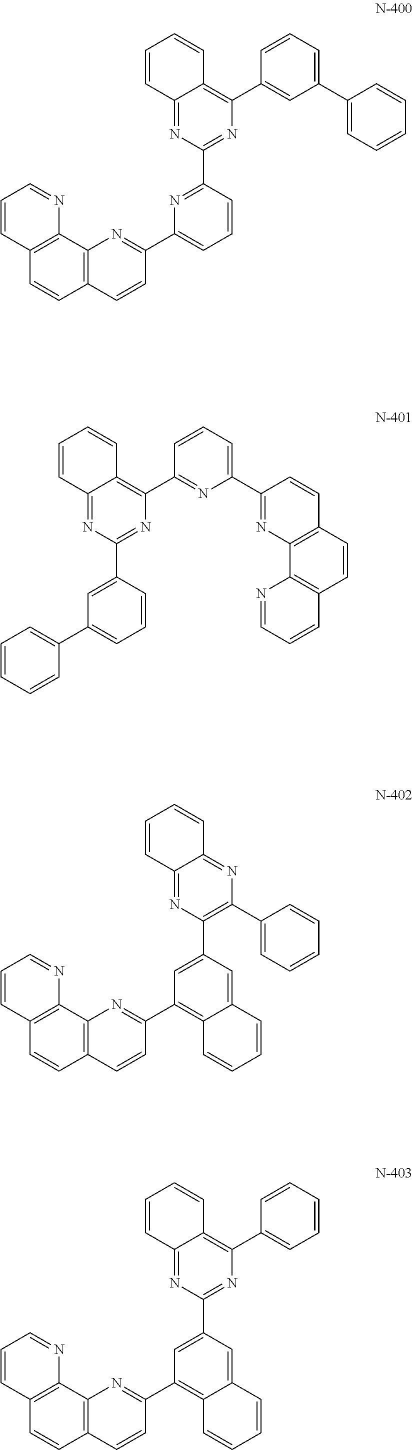 [SMILES: c1ccc(-c2cccc(-c3nc(-c4cccc(-c5ccc6ccc7cccnc7c6n5)n4)nc4ccccc34)c2)cc1 ; c1ccc(-c2cccc(-c3nc(-c4cccc(-c5ccc6ccc7cccnc7c6n5)n4)c4ccccc4n3)c2)cc1 ; c1ccc(-c2nc3ccccc3nc2-c2cc(-c3ccc4ccc5cccnc5c4n3)c3ccccc3c2)cc1 ; c1ccc(-c2nc(-c3cc(-c4ccc5ccc6cccnc6c5n4)c4ccccc4c3)nc3ccccc23)cc1]

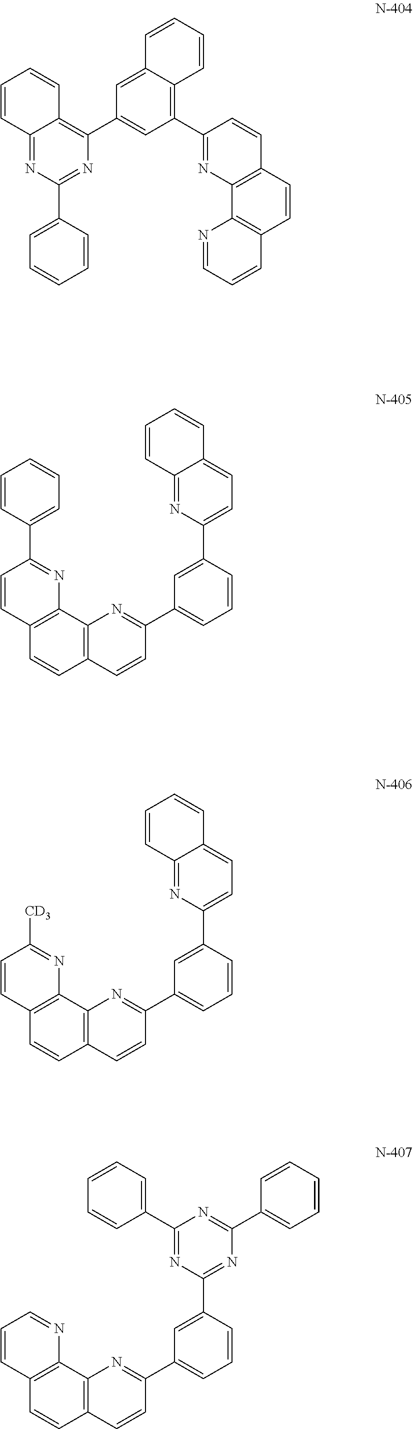 [SMILES: c1ccc(-c2nc(-c3cc(-c4ccc5ccc6cccnc6c5n4)c4ccccc4c3)c3ccccc3n2)cc1 ; c1ccc(-c2ccc3ccc4ccc(-c5cccc(-c6ccc7ccccc7n6)c5)nc4c3n2)cc1 ; [2H]C([2H])([2H])c1ccc2ccc3ccc(-c4cccc(-c5ccc6ccccc6n5)c4)nc3c2n1 ; c1ccc(-c2nc(-c3ccccc3)nc(-c3cccc(-c4ccc5ccc6cccnc6c5n4)c3)n2)cc1]

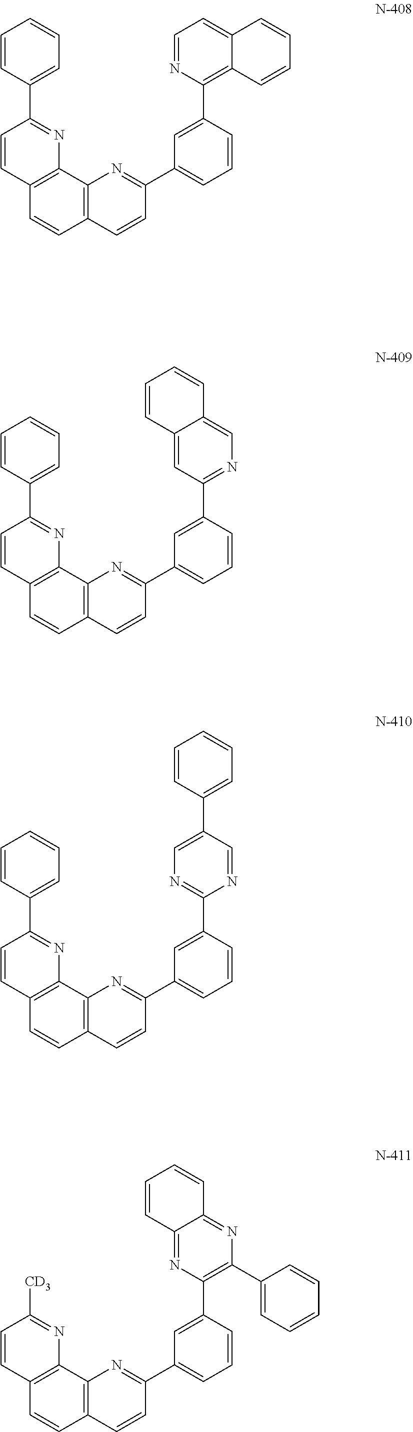 [SMILES: c1ccc(-c2ccc3ccc4ccc(-c5cccc(-c6nccc7ccccc67)c5)nc4c3n2)cc1 ; c1ccc(-c2ccc3ccc4ccc(-c5cccc(-c6cc7ccccc7cn6)c5)nc4c3n2)cc1 ; c1ccc(-c2cnc(-c3cccc(-c4ccc5ccc6ccc(-c7ccccc7)nc6c5n4)c3)nc2)cc1 ; [2H]C([2H])([2H])c1ccc2ccc3ccc(-c4cccc(-c5nc6ccccc6nc5-c5ccccc5)c4)nc3c2n1]

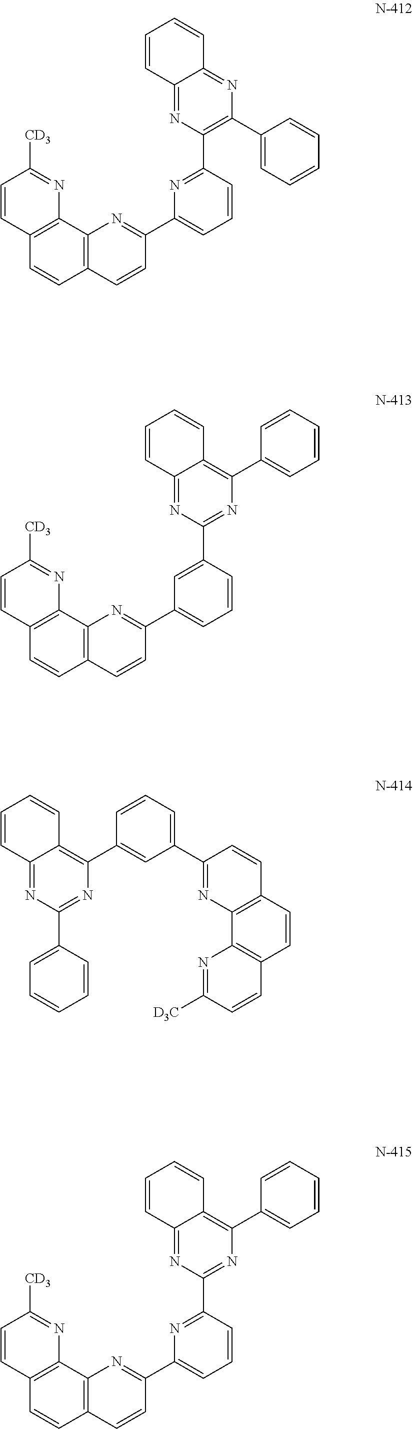 [SMILES: [2H]C([2H])([2H])c1ccc2ccc3ccc(-c4cccc(-c5nc6ccccc6nc5-c5ccccc5)n4)nc3c2n1 ; [2H]C([2H])([2H])c1ccc2ccc3ccc(-c4cccc(-c5nc(-c6ccccc6)c6ccccc6n5)c4)nc3c2n1 ; [2H]C([2H])([2H])c1ccc2ccc3ccc(-c4cccc(-c5nc(-c6ccccc6)nc6ccccc56)c4)nc3c2n1 ; [2H]C([2H])([2H])c1ccc2ccc3ccc(-c4cccc(-c5nc(-c6ccccc6)c6ccccc6n5)n4)nc3c2n1]

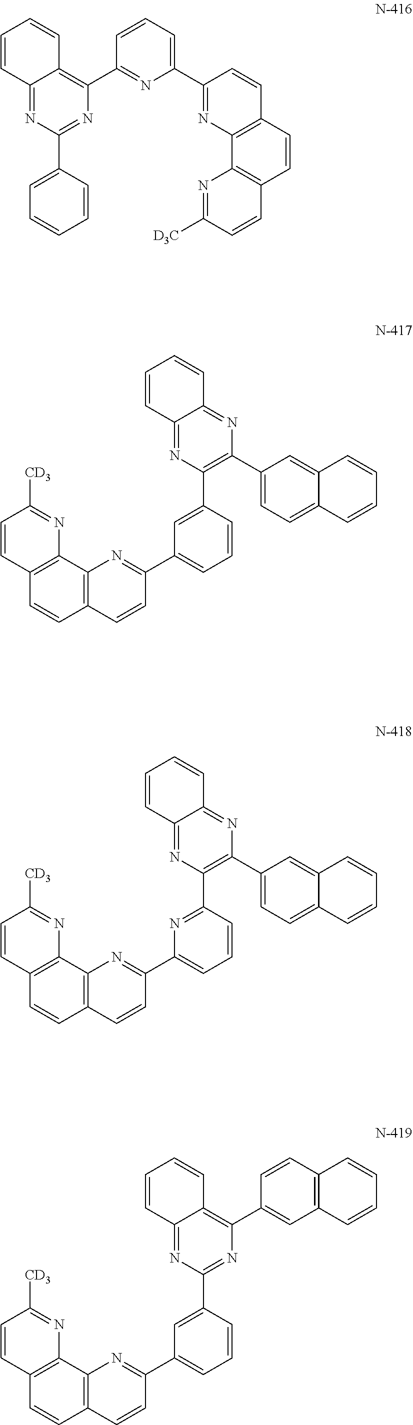 [SMILES: [2H]C([2H])([2H])c1ccc2ccc3ccc(-c4cccc(-c5nc(-c6ccccc6)nc6ccccc56)n4)nc3c2n1 ; [2H]C([2H])([2H])c1ccc2ccc3ccc(-c4cccc(-c5nc6ccccc6nc5-c5ccc6ccccc6c5)c4)nc3c2n1 ; [2H]C([2H])([2H])c1ccc2ccc3ccc(-c4cccc(-c5nc6ccccc6nc5-c5ccc6ccccc6c5)n4)nc3c2n1 ; [2H]C([2H])([2H])c1ccc2ccc3ccc(-c4cccc(-c5nc(-c6ccc7ccccc7c6)c6ccccc6n5)c4)nc3c2n1]

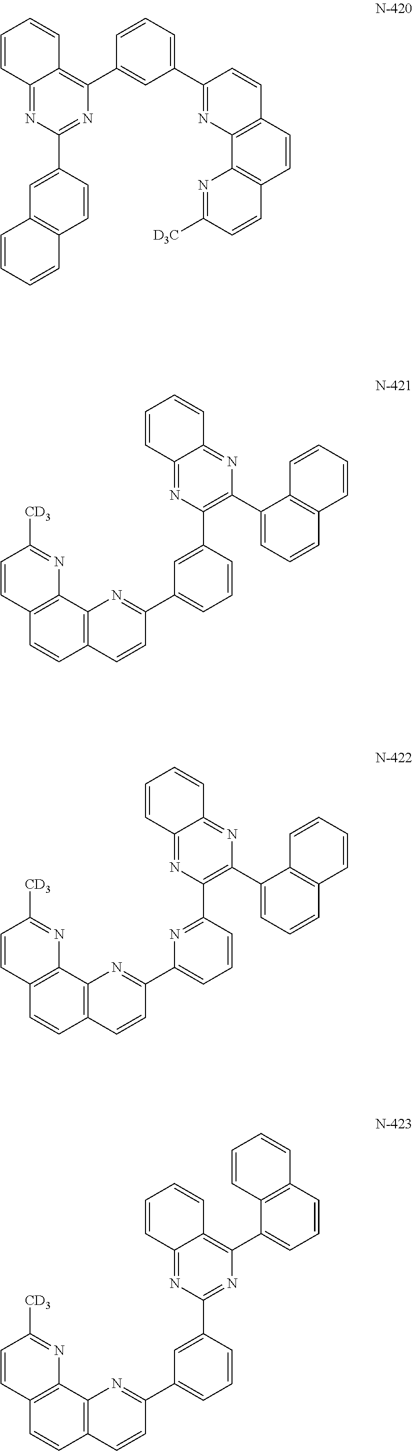 [SMILES: [2H]C([2H])([2H])c1ccc2ccc3ccc(-c4cccc(-c5nc(-c6ccc7ccccc7c6)nc6ccccc56)c4)nc3c2n1 ; [2H]C([2H])([2H])c1ccc2ccc3ccc(-c4cccc(-c5nc6ccccc6nc5-c5cccc6ccccc56)c4)nc3c2n1 ; [2H]C([2H])([2H])c1ccc2ccc3ccc(-c4cccc(-c5nc6ccccc6nc5-c5cccc6ccccc56)n4)nc3c2n1 ; [2H]C([2H])([2H])c1ccc2ccc3ccc(-c4cccc(-c5nc(-c6cccc7ccccc67)c6ccccc6n5)c4)nc3c2n1]

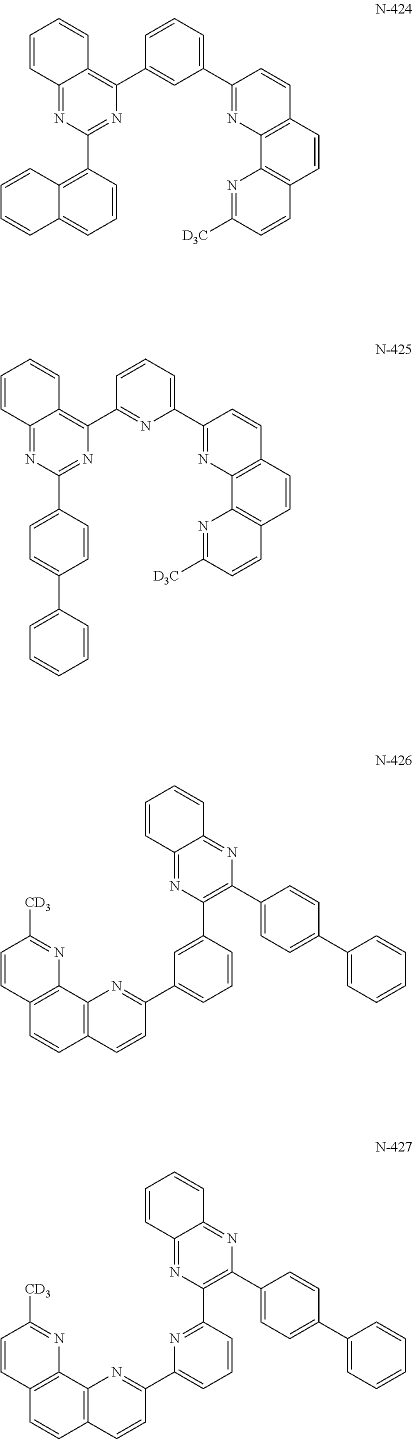 [SMILES: [2H]C([2H])([2H])c1ccc2ccc3ccc(-c4cccc(-c5nc(-c6cccc7ccccc67)nc6ccccc56)c4)nc3c2n1 ; [2H]C([2H])([2H])c1ccc2ccc3ccc(-c4cccc(-c5nc(-c6ccc(-c7ccccc7)cc6)nc6ccccc56)n4)nc3c2n1 ; [2H]C([2H])([2H])c1ccc2ccc3ccc(-c4cccc(-c5nc6ccccc6nc5-c5ccc(-c6ccccc6)cc5)c4)nc3c2n1 ; [2H]C([2H])([2H])c1ccc2ccc3ccc(-c4cccc(-c5nc6ccccc6nc5-c5ccc(-c6ccccc6)cc5)n4)nc3c2n1]

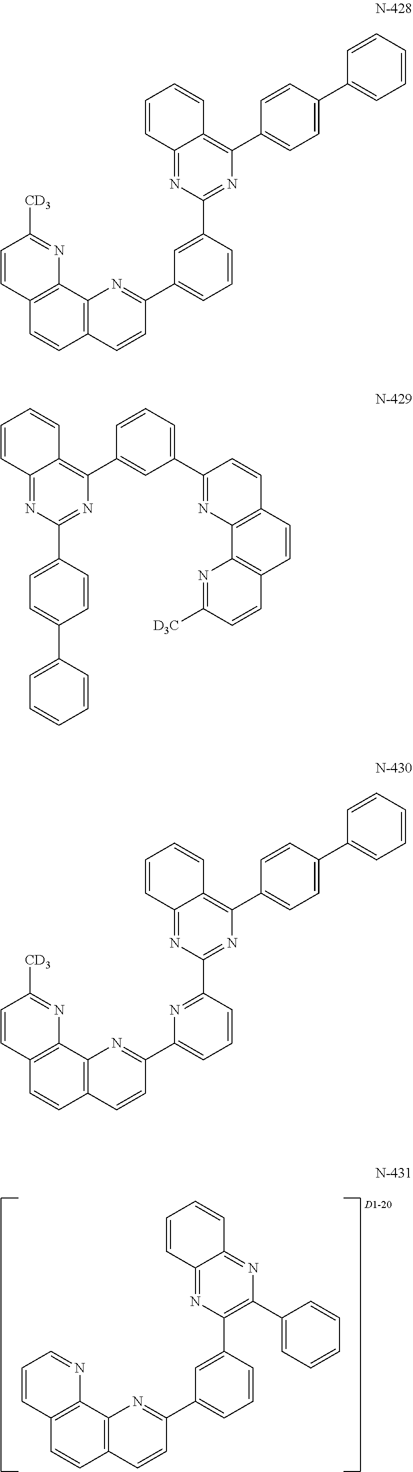

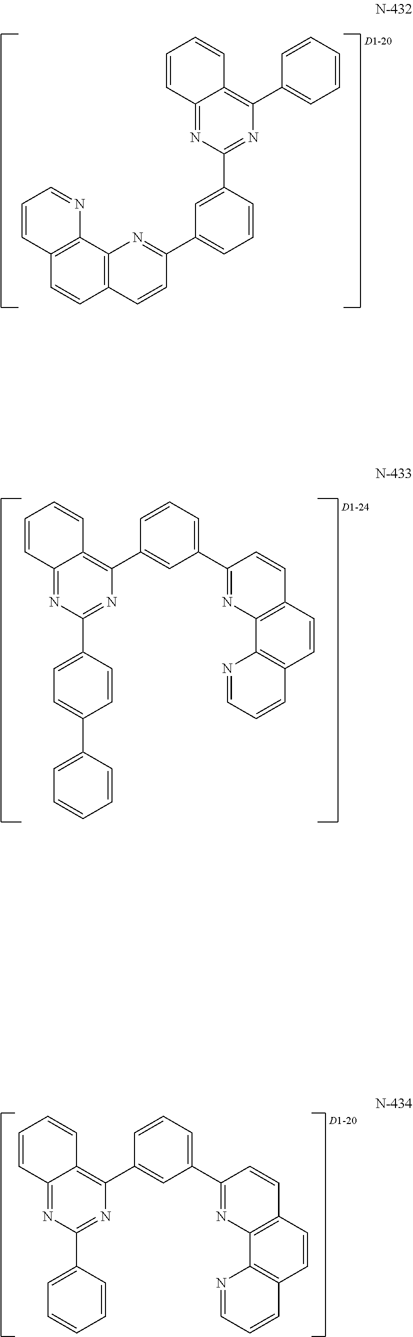

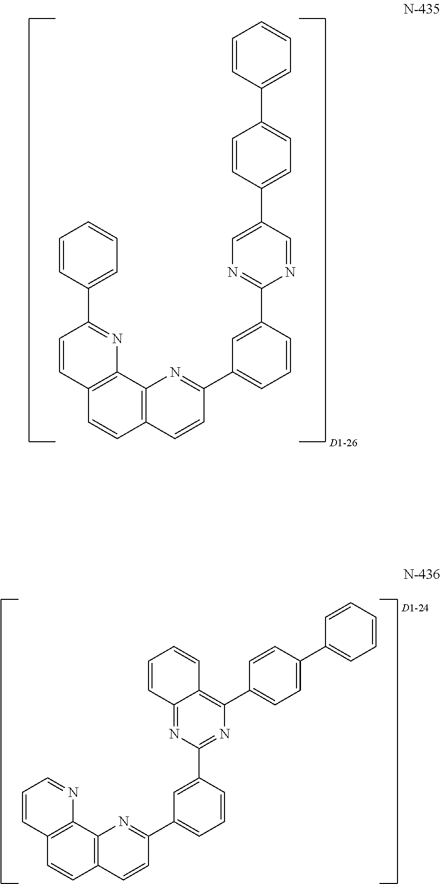

In the compounds above, Dn means that n number of hydrogens is replaced with deuterium, wherein n represents an integer of 1 or more, wherein the upper limit of n is determined by the number of hydrogens that can be substituted in each compound.

According to another embodiment, the present disclosure provides an organic electroluminescent device to which the organic electroluminescent material comprising the aforementioned compound represented by Formulas 1-1 to 1-4, and Formulas 2 and 2-1 is applied.

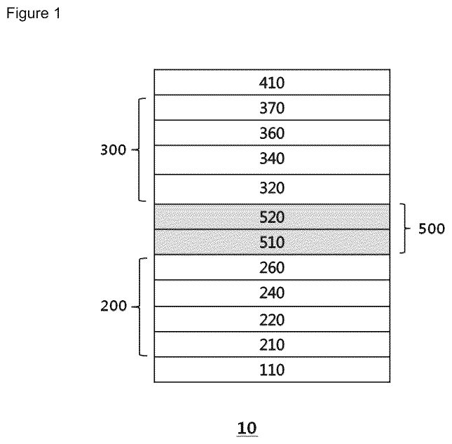

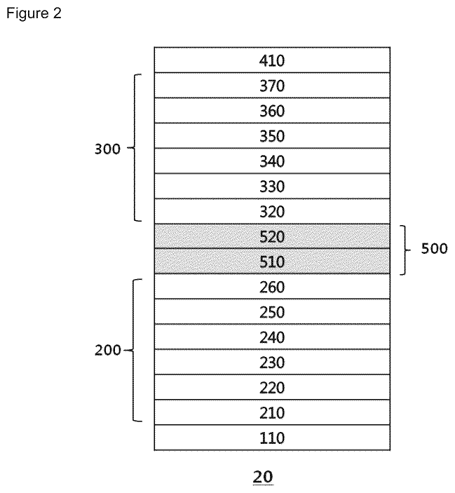

FIGS. 1 and 2 show an example of an organic electroluminescent device according to one embodiment, respectively. In the present disclosure, “first” and “second” are added for convenience to refer to each layer included in a plurality of light-emitting units, and expressions such as “first” and “second” may be omitted to describe common functions.

Hereinafter, an organic electroluminescent device to which the aforementioned organic electroluminescent material is applied will be described with reference to the drawings.

As shown in FIG. 1, an organic electroluminescent device 10 according to one embodiment includes a first electrode 110, a second electrode 410 opposite the first electrode 110, a plurality of light-emitting units 200 and 300 located between the first electrode 110 and the second electrode 410, and at least one charge generation layer 500 located between adjacent light-emitting units 200 and 300. The light-emitting units 200 and 300 include at least one light-emitting layers 240 and 340, and the charge generation layer 500 includes an N-type charge generation layer 510 and a P-type charge generation layer 520. Wherein, the light-emitting layers 240 and 340 include the compound represented by Formula 1-1, and the N-type charge generation layer 510 includes the compound represented by Formula 2. Alternatively, the light-emitting layers 240 and 340 include at least two of the compounds represented by Formulas 1-2 to 1-4, and the N-type charge generation layer 510 includes the compound represented by Formula 2.

The organic electroluminescent device according to one embodiment includes at least two light-emitting units, and the charge generation layer may be positioned between adjacent light-emitting units to add the number of light-emitting units.

According to one embodiment, the light-emitting units 200 and 300 includes the hole transport layers 220 and 320, the light-emitting layers 240 and 340, and the electron transport layers 260 and 360, respectively. Specifically, the first light-emitting unit 200 includes a hole transport layer 220, a first light-emitting layer 240, and an electron transport layer 260, and the second light-emitting unit 300 includes a hole transport layer 320, a second light-emitting layer 340, and an electron transport layer 360. Additionally, the first light-emitting unit 200 additionally includes a hole injection layer 210, and the second light-emitting unit 300 additionally includes an electron injection layer 370.

The light-emitting layers 240 and 340 are a layer in which light is emitted including a host and a dopant, and may be a single layer or a plurality of layers in which two or more layers are stacked. Wherein, the host mainly promotes recombination of electrons and holes, and has a function of confining excitons in the light-emitting layer, and the dopant has a function of efficiently emitting excitons obtained through recombination. The dopant compound of the light-emitting layers 240 and 340 may be doped in an amount of less than 25% by weight, preferably, less than 17% by weight, more preferably, less than 10% by weight with respect to the total amount of host compound and the dopant compound.

According to one embodiment, the light-emitting layers 240 and 340 include an anthracene derivative compound represented by Formula 1-1 as a host material. In one embodiment, at least one of the light-emitting layers may include a deuterated compound represented by Formula 1-1, preferably, one of the light-emitting layers may include a deuterated compound represented by Formula 1-1. This results in increased device stability and improved lifespan characteristics.

According to another embodiment, the light-emitting layers 240 and 340 include at least two host compounds of Formulas 1-2 to 1-4 as host materials. In one embodiment, at least one of the light-emitting layers includes a deuterated compound represented by Formula 1-2 and a deuterated compound represented by Formula 1-4, or, for example, a deuterated compound represented by Formula 1-2 and a deuterated compound represented by 1-3, or a deuterated compound represented by Formula 1-3 and a deuterated compound represented by Formula 1-4.

According to one embodiment, the charge generation layer 500 includes an N-type charge generation layer 510 located adjacent to the first light-emitting unit 200 and supplying electrons to the first light-emitting unit 200, and a P-type charge generation layer 520 located adjacent to the second light-emitting unit 300 and supplying holes to the second light-emitting unit 300.

The N-type charge generation layer 510 includes the compound represented by Formula 2. The compound of Formula 2 has excellent electron mobility and thus has excellent electron injection and transfer capabilities. Therefore, when the compound of Formula 2 is applied to an organic electroluminescent device as an N-type charge generation layer material, an increase in the progressive driving voltage of the device and a decrease in its lifespan can be prevented.

According to one embodiment, the N-type charge generation layer 510 may further include an N-type dopant to improve electron injection characteristics into the N-type charge generation layer. For example, usable N-type dopants may further include alkali metals such as Li, Na, K, Rb, Cs, Fr, etc., alkaline earth metals such as Be, Mg, Ca, Sr, Ba, Ra, etc., or one or more complex compounds such metals which is generally in the art. In the N-type charge generation layer 510, the doping concentration of the dopant may be 0.5% to 10% of the compound of Formula 2.

The P-type charge generation layer 520 may be used as a hole injection layer, and may include the hole injection layer material alone or a mixture of the hole injection layer material and the hole transfer material.

At least one of the light-emitting layers 240 and 340 and the N-type charge generation layer 510 in the present disclosure includes a deuterated compound. Specifically, at least one of the light-emitting layers 240 and 340 included in the light-emitting units 200 and 300 includes the deuterated compounds represented by Formula 1-1 or at least two of the compounds represented by Formulas 1-2 to 1-4 and/or the N-type charge generation layer 510 includes a deuterated compound represented by Formula 2.

One of the first electrode 110 and the second electrode 410 may be an anode and the other may be a cathode. Wherein, the first electrode 110 and the second electrode 410 may each be formed as a transmissive conductive material, a transflective conductive material, or a reflective conductive material. The organic electroluminescent device may be a top emission type, a bottom emission type, or a both-sides emission type according to the kinds of the material forming the first electrode 110 and the second electrode 410.

Referring to FIG. 2, the light-emitting units 200 and 300 according to one embodiment may further include hole blocking layers 250 and 350 between the light-emitting layers 240 and 340 and the electron transport layers 260 and 360.

The hole blocking layers 250 and 350 are layers that prevent holes from reaching the cathode, thereby improving the probability of recombination of electrons and holes in the light-emitting layer. The hole blocking layers 250 and 350 or the electron transport layers 260 and 360 may be comprised of a plurality of layers, and a plurality of compounds may be used for each layer. Additionally, the electron injection layers 260 and 360 may be doped with n-dopants.

According to one embodiment, the hole blocking layers 250 and 350, the electron transport layers 260 and 360, and the N-type charge generation layer 510 may include a compound represented by Formula 2-1 according to the present disclosure.

The hole injection layer 210 may be a plurality of layers for the purpose of lowering the hole injection barrier (or hole injection voltage) from the anode to the hole transport layers 220 and 230 or the electron blocking layer. For each layer, two compounds can be used simultaneously. Additionally, the hole injection layer 210 may be doped with p-dopant. In addition, although it is not shown in the present disclosure, the electron blocking layer is located between the hole transport layer (or hole injection layer) and the light-emitting layers 240 and 340 to block the overflow of electrons from the light-emitting layer and trap excitons within the light-emitting layer to prevent light leakage. The hole transport layers 220, 230, 320, and 330 or the electron blocking layer may be comprised of a plurality of layers, and a plurality of compounds may be used in each layer. When an organic electroluminescent device includes two or more hole transport layers, the hole transport layer, which is further included, may be used as the hole auxiliary layer or the electron blocking layer.

The organic electroluminescent device according to one embodiment may include a light-emitting auxiliary layer placed between the anode and the light-emitting layer, or between the cathode and the light-emitting layer. When the light-emitting auxiliary layer is placed between the anode and the light-emitting layer, it can be used for promoting the hole injection and/or the hole transport, or for preventing the overflow of electrons. When the light-emitting auxiliary layer is placed between the cathode and the light-emitting layer, it can be used for promoting the electron injection and/or the electron transport, or for preventing the overflow of holes. In addition, the organic electroluminescent device according to one embodiment may further include the hole auxiliary layer placed between the hole transport layer (or hole injection layer) and the light-emitting layer. The hole auxiliary layer may be effective to promote or block the hole transport rate (or the hole injection rate), thereby enabling the charge balance to be controlled.

In the organic electroluminescent device of the present disclosure, preferably, at least one layer (hereinafter, “a surface layer”) selected from a chalcogenide layer, a halogenated metal layer, and a metal oxide layer may be placed on an inner surface(s) of one or both of a pair of electrodes. Specifically, a chalcogenide (including oxides) layer of silicon and aluminum is preferably placed on an anode surface of an electroluminescent medium layer, and a halogenated metal layer or a metal oxide layer is preferably placed on a cathode surface of an electroluminescent medium layer. The operation stability for the organic electroluminescent device may be obtained by the surface layer. Preferably, the chalcogenide includes SiO_{X} (1≤X≤2), AlO_{X} (1≤X≤1.5), SiON, SiAlON, etc.; the halogenated metal includes LiF, MgF₂, CaF₂, a rare earth metal fluoride, etc.; and the metal oxide includes Cs₂O, Li₂O, MgO, SrO, BaO, CaO, etc.

An organic electroluminescent device according to one embodiment may further comprise at least one dopant in the light-emitting layers 240 and 340. The dopant comprised in the organic electroluminescent device of the present disclosure may be at least one phosphorescent or fluorescent dopant, preferably a phosphorescent dopant. The phosphorescent dopant material applied to the organic electroluminescent device of the present disclosure is not particularly limited, but may be preferably a metallated complex compound(s) of a metal atom(s) selected from iridium (Ir), osmium (Os), copper (Cu), and platinum (Pt), more preferably an ortho-metallated complex compound(s) of a metal atom(s) selected from iridium (Ir), osmium (Os), copper (Cu), and platinum (Pt), and even more preferably ortho-metallated iridium complex compound(s).

The dopant comprised in the organic electroluminescent device of the present disclosure may use the compound represented by the following Formula 100, but is not limited thereto.

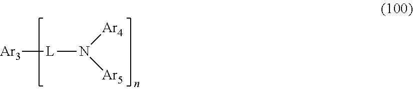

In Formula 100,
L represents a single bond, a substituted or unsubstituted (C6-C30)arylene, or a substituted or unsubstituted (3- to 30-membered) heteroarylene;
Ar₄ and Ar₅ each independently represent, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring of (C3-C30) aliphatic ring and (C6-C30) aromatic ring, or -L₄-N(Ar₁₃)(Ar₁₄); or Ar₄ and Ar₅ may be linked to each other to form a ring(s);
n is an integer of 0 to 2, when n is 0, Ar₃ is represented by the following Formula 100-1, and when n is 2, each of

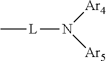

may be the same or different; and
Ar₃ is represented b an one of the following Formulas 100-1 to 100-5;

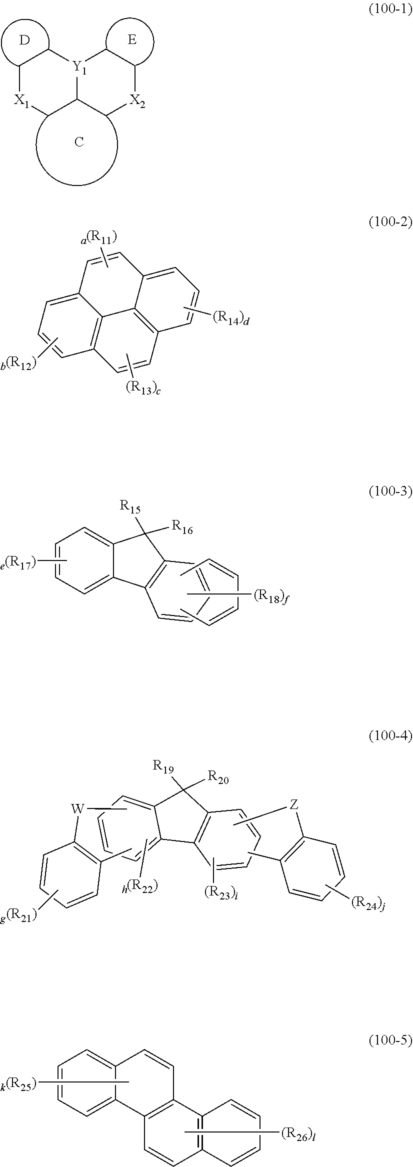

In Formulas 100-1 to 100-5,
Y₁ represents B;
X₁ and X₂ each independently represent, NR′, O, or S;
W and Z each independently represent, O, S, NR′, or CR₂₇R₂₈;
R′ represents hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring of (C3-C30) aliphatic ring and (C6-C30) aromatic ring, or -L₄-N(Ar₁₃)(Ar₁₄), or R′ may be directly linked to at least one of Ring C. Ring D, and Ring E, or may be linked via B, O, S or CR₂₇R₂₈ as a linker to form a ring(s);
Ring C, Ring D, and Ring E each independently represent, a substituted or unsubstituted (C6-C30)aryl, or a substituted or unsubstituted (3- to 50-membered)heteroaryl; or Ring D and Ring E may be directly linked to each other, or may be linked via B, O, S or CR₂₇R₂₈ as a linker to form a ring(s);
R₁₁ to R₁₄, R₁₇, R₁₈, and R₂₁ to R₂₈ each independently represent, hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsiyl, a substituted or unsubstituted tri(C6-C30)arylsilyl, a substituted or unsubstituted fused ring of (C3-C30) aliphatic ring and (C6-C30) aromatic ring, or -L₄-N(Ar₁₃)(Ar₁₄);
R₁₅, R₁₆, R₁₉, R₂₀, R₂₇, and R₂₈ each independently represent, a substituted or unsubstituted (C1-C30)alkyl, a substituted or unsubstituted (C6-C30)aryl, or a substituted or unsubstituted (3- to 30-membered)heteroaryl, or at least one of R₁₅ and R₁₆, R₁₉, and R₂₀, and R₂₇ and R₂₈ may be fused to each other to form a spiro structure(s);
L₄ represent a single bond, a substituted or unsubstituted (C6-C30)aryl, a substituted or unsubstituted (3- to 30-membered)heteroaryl, or a substituted or unsubstituted (C2-C30) divalent aliphatic hydrocarbon group, or a substituted or unsubstituted fused ring of divalent (C3-C30) aliphatic ring and (C6-C30) aromatic ring;
Ar₁₃ and Ar₁₄ each independently represent, a substituted or unsubstituted (C1-C30)alkyl, a substituted or unsubstituted (C2-C30)alkenyl, a substituted or unsubstituted (C6-C30)aryl, or a substituted or unsubstituted (3- to 30-membered)heteroaryl;
a, c, h, and i each independently represent, an integer of 1 or 2, b and d each independently represent, an integer of 1 to 3, f, k, and l each independently represent, an integer of 1 to 6, and e, g, and j each independently represent, an integer of 1 to 4;
when a to 1 are 2 or more, each of R₁₁ to R₁₄, R₁₇, R₁₈, and R₂₁ to R₂₈ may be the same or different; and
Ring C, Ring D, Ring E, and R₁₁ to R₁₄, R₁₇, R₁₈, and R₂₁ to R₂₈ may have a position connected to L in Formula 100.

According to one embodiment, the compound represented by Formula 100 may be more specifically illustrated by the following compounds, but is not limited thereto.

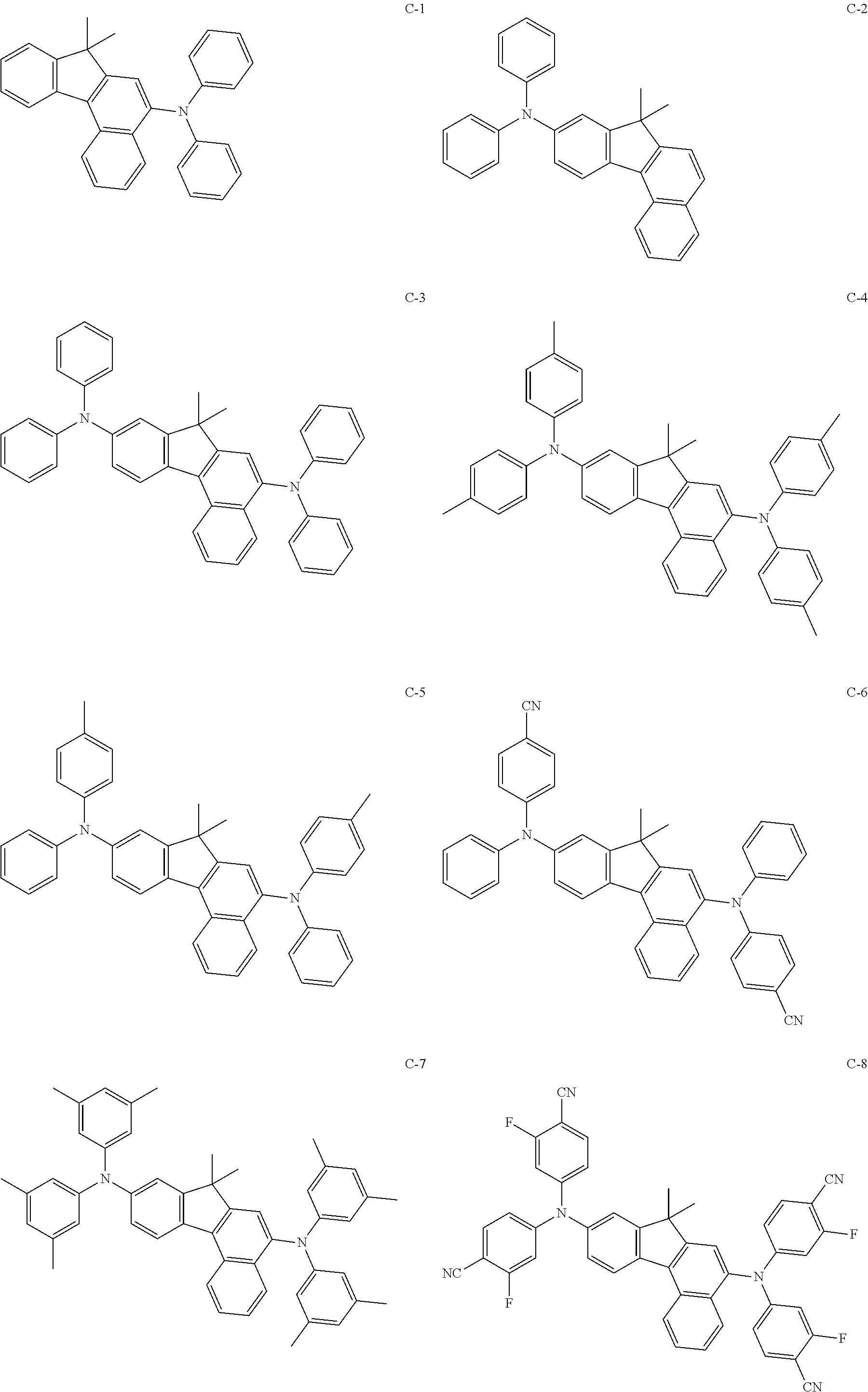

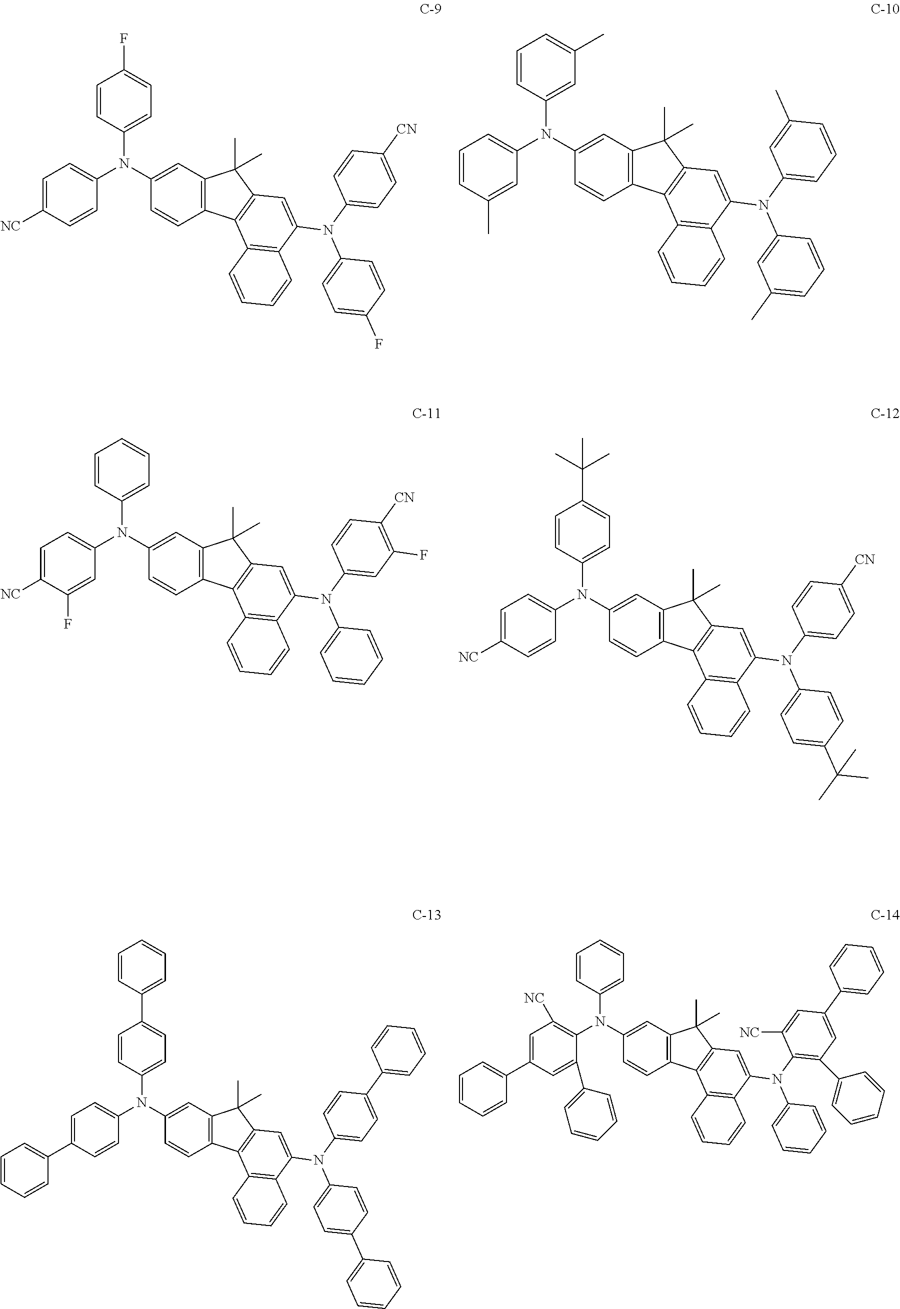

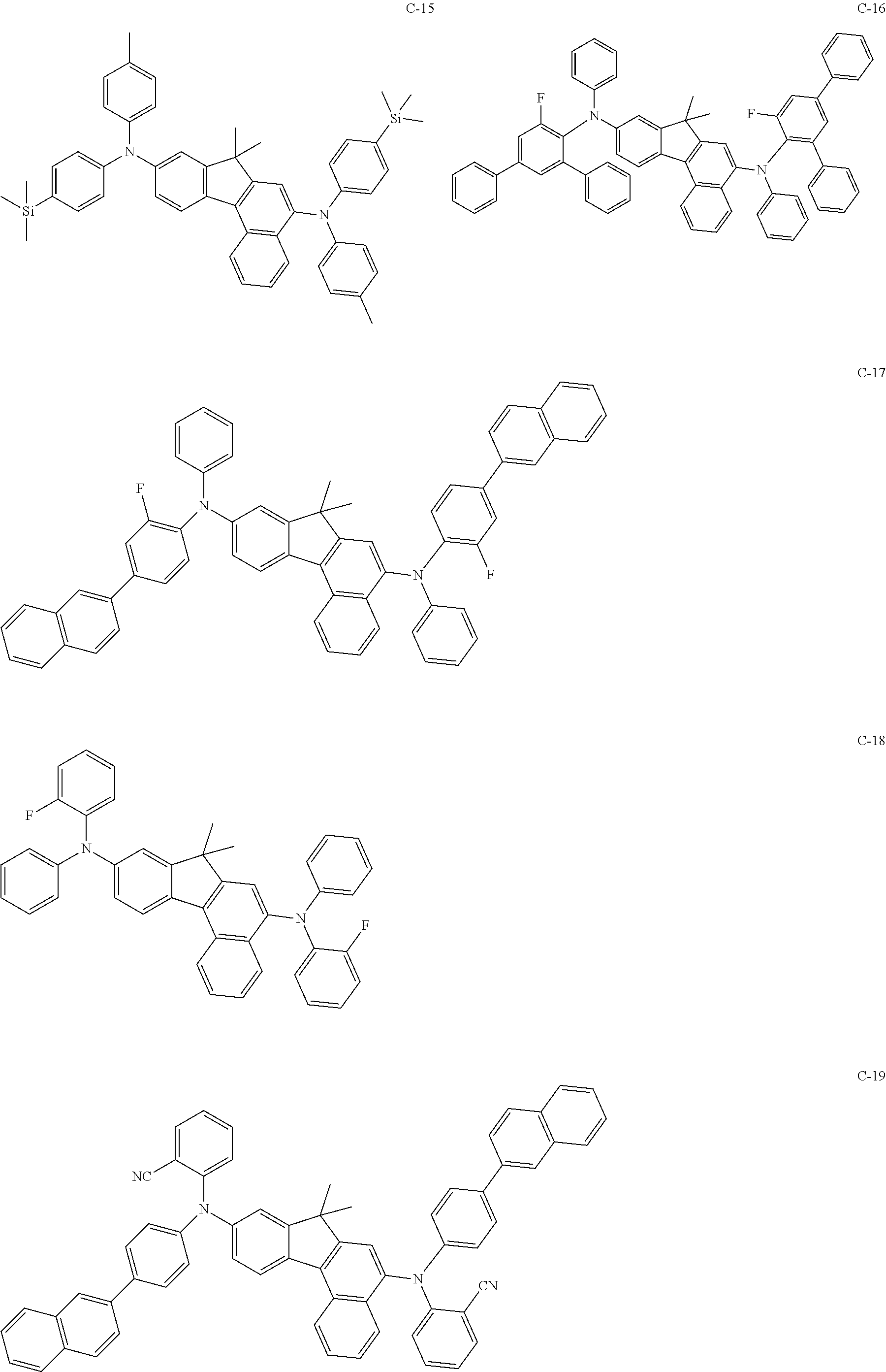

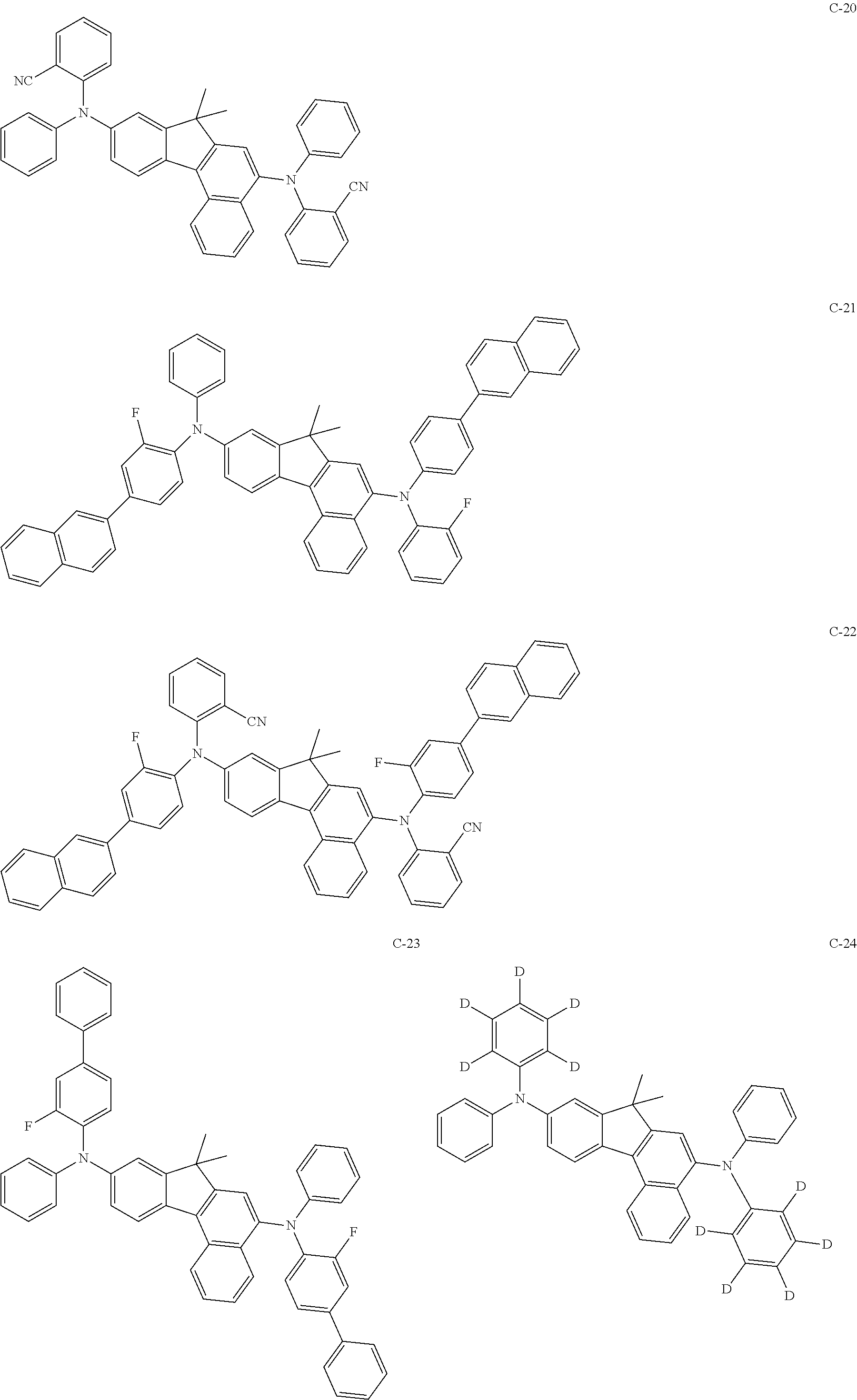

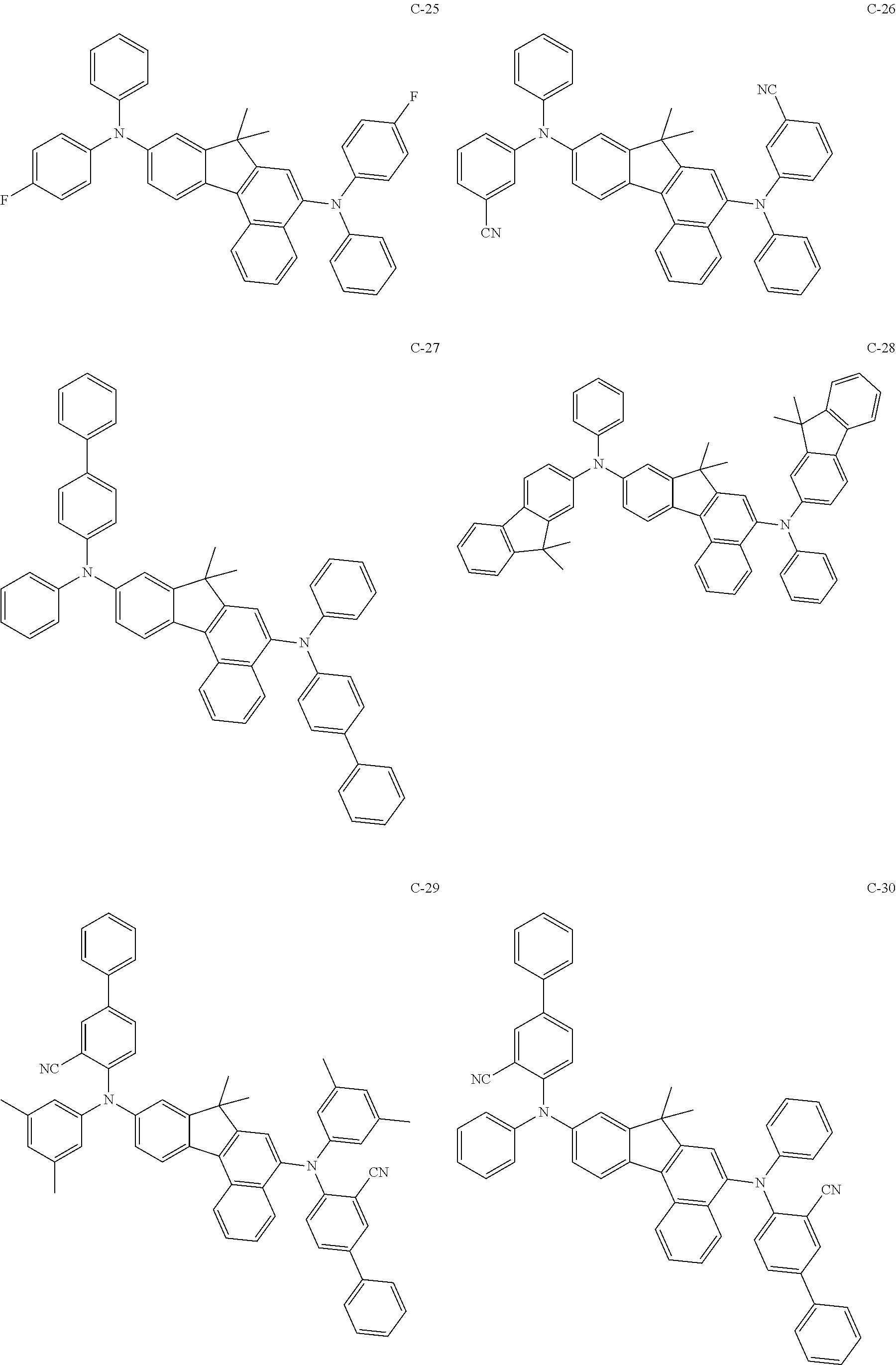

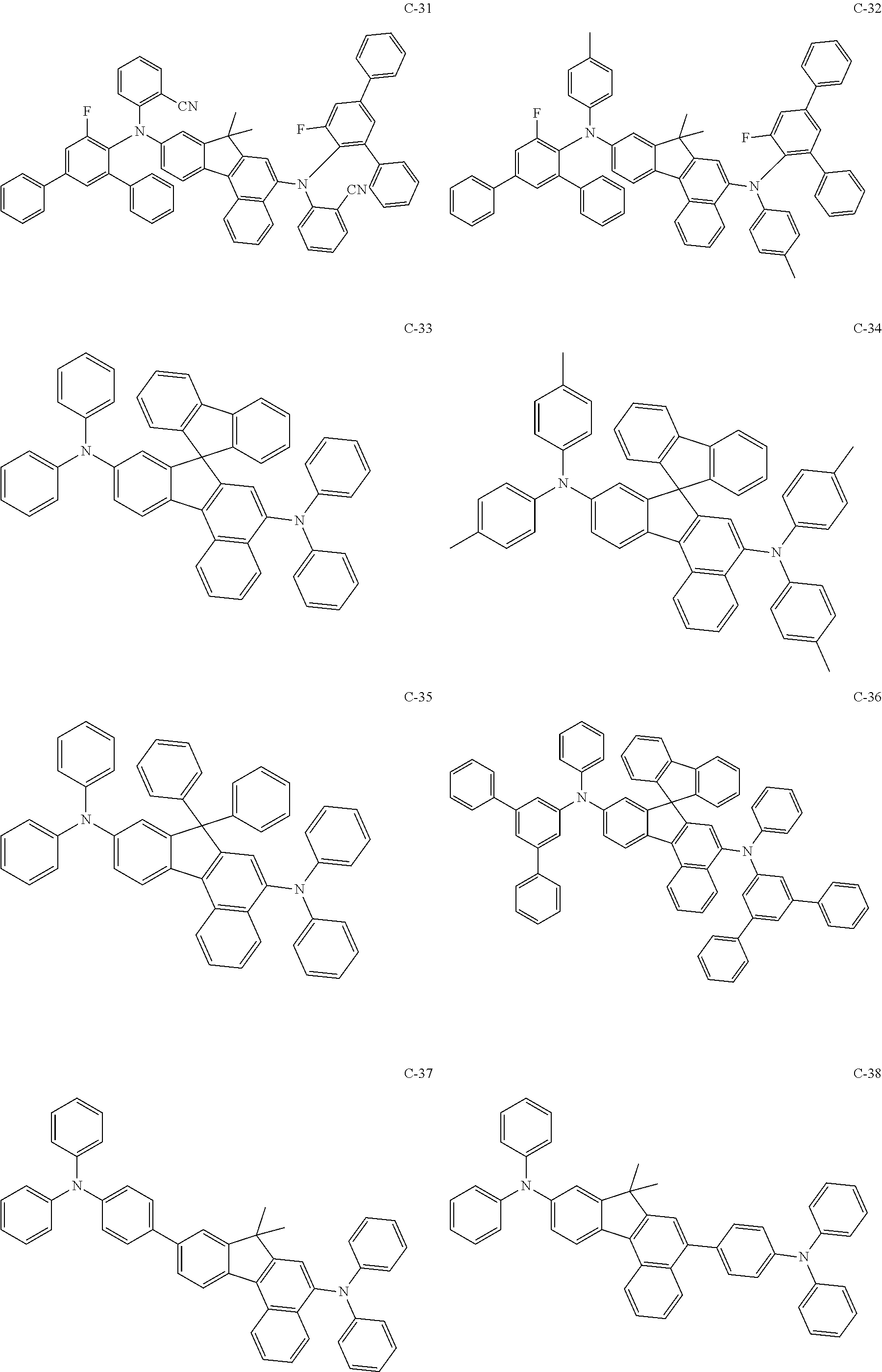

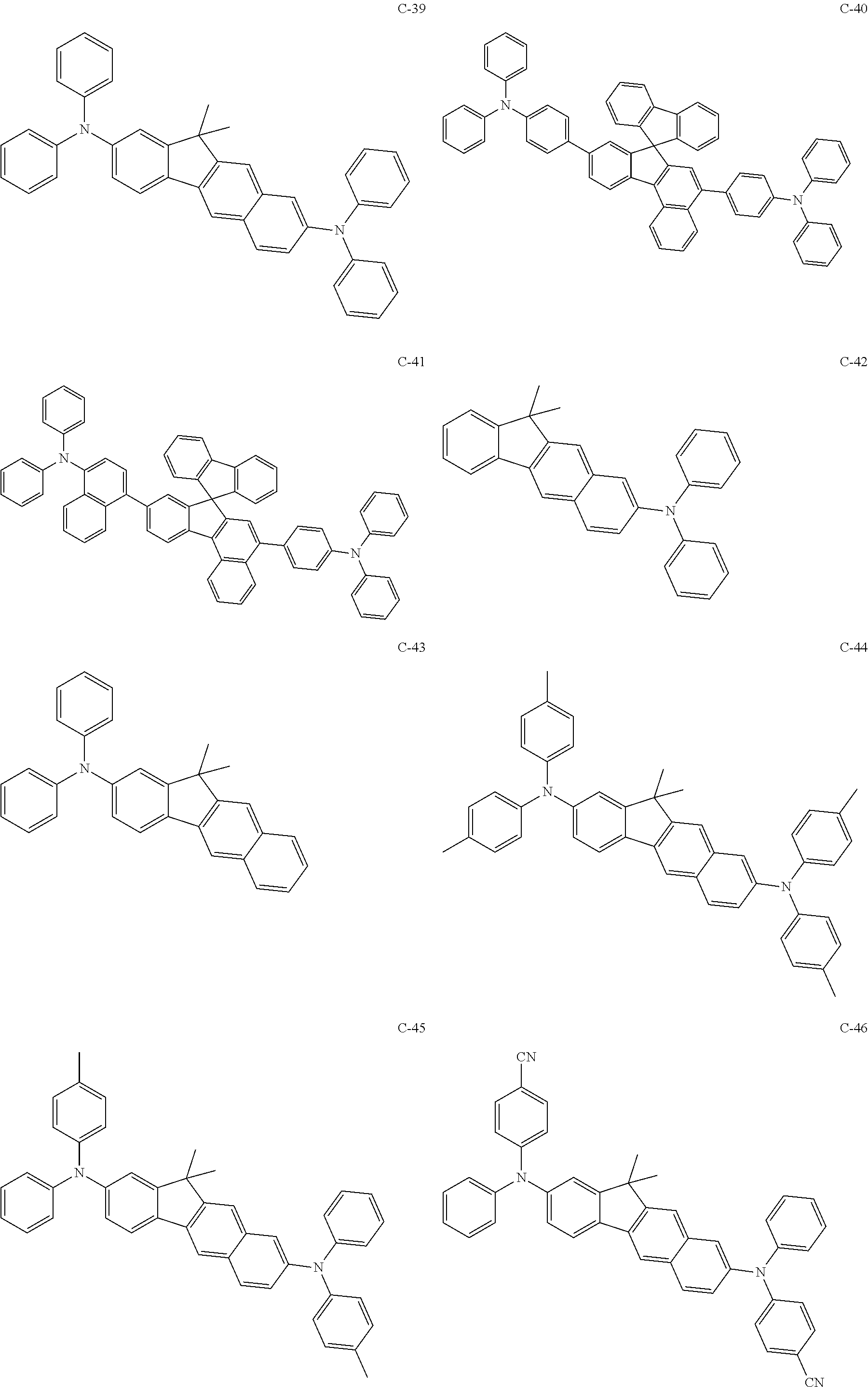

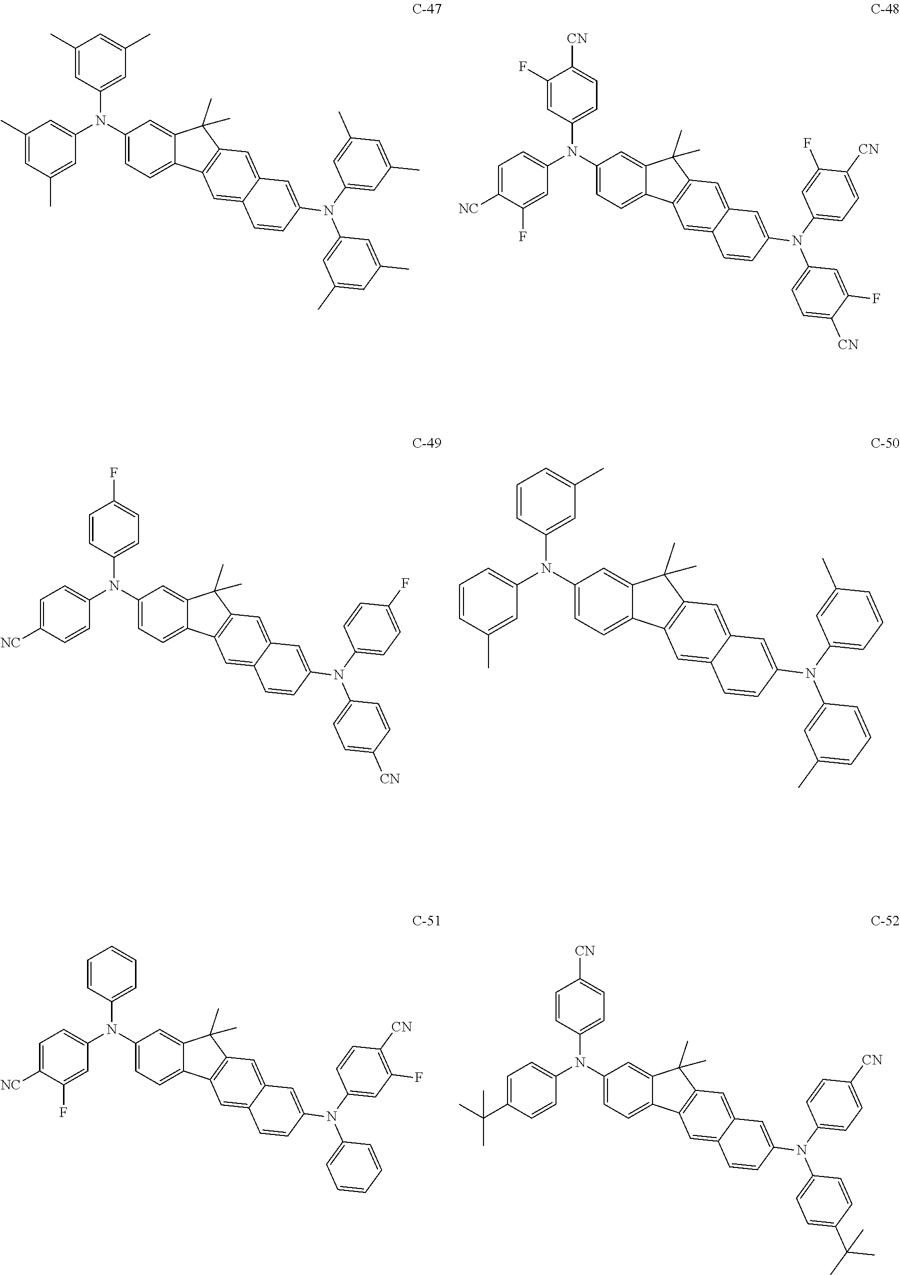

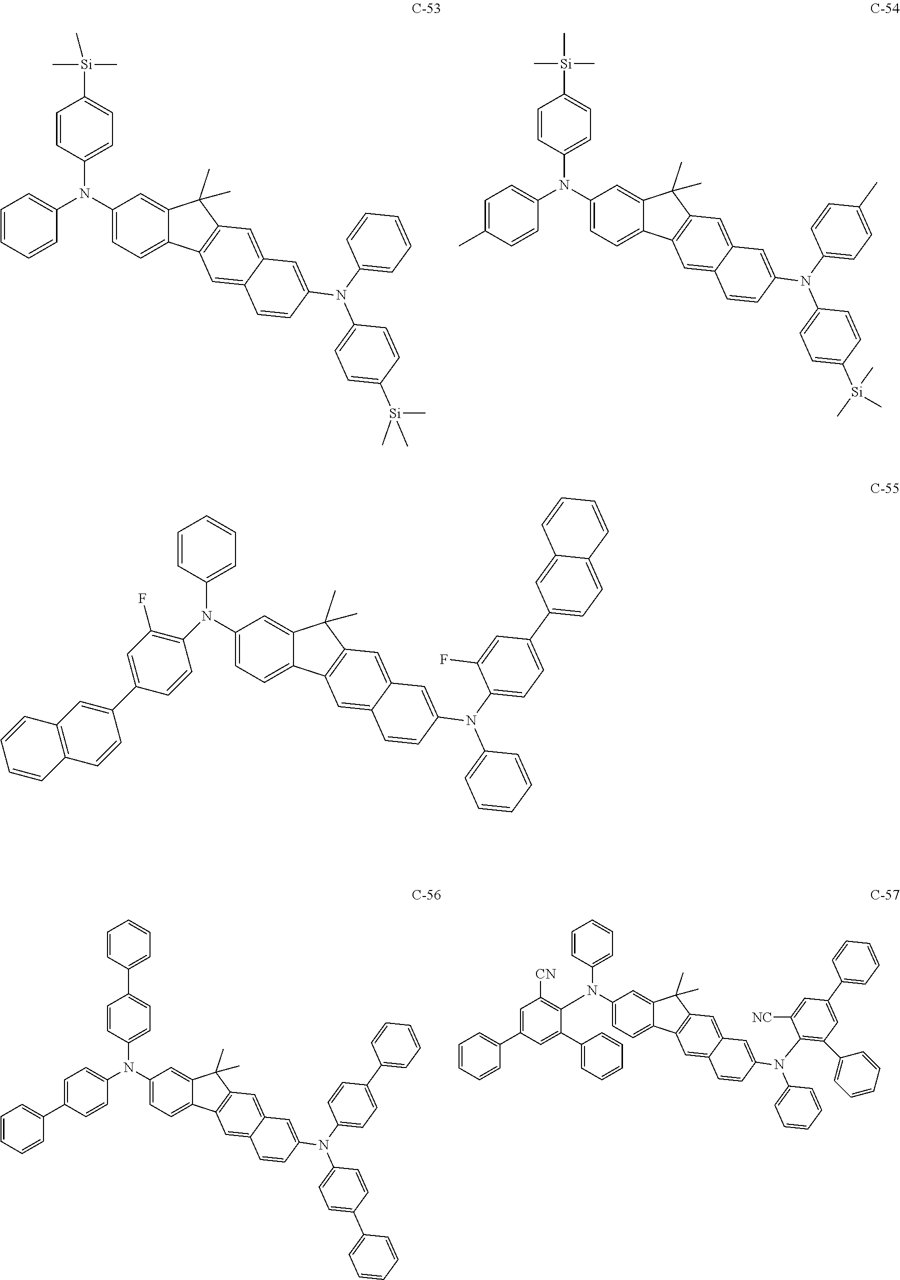

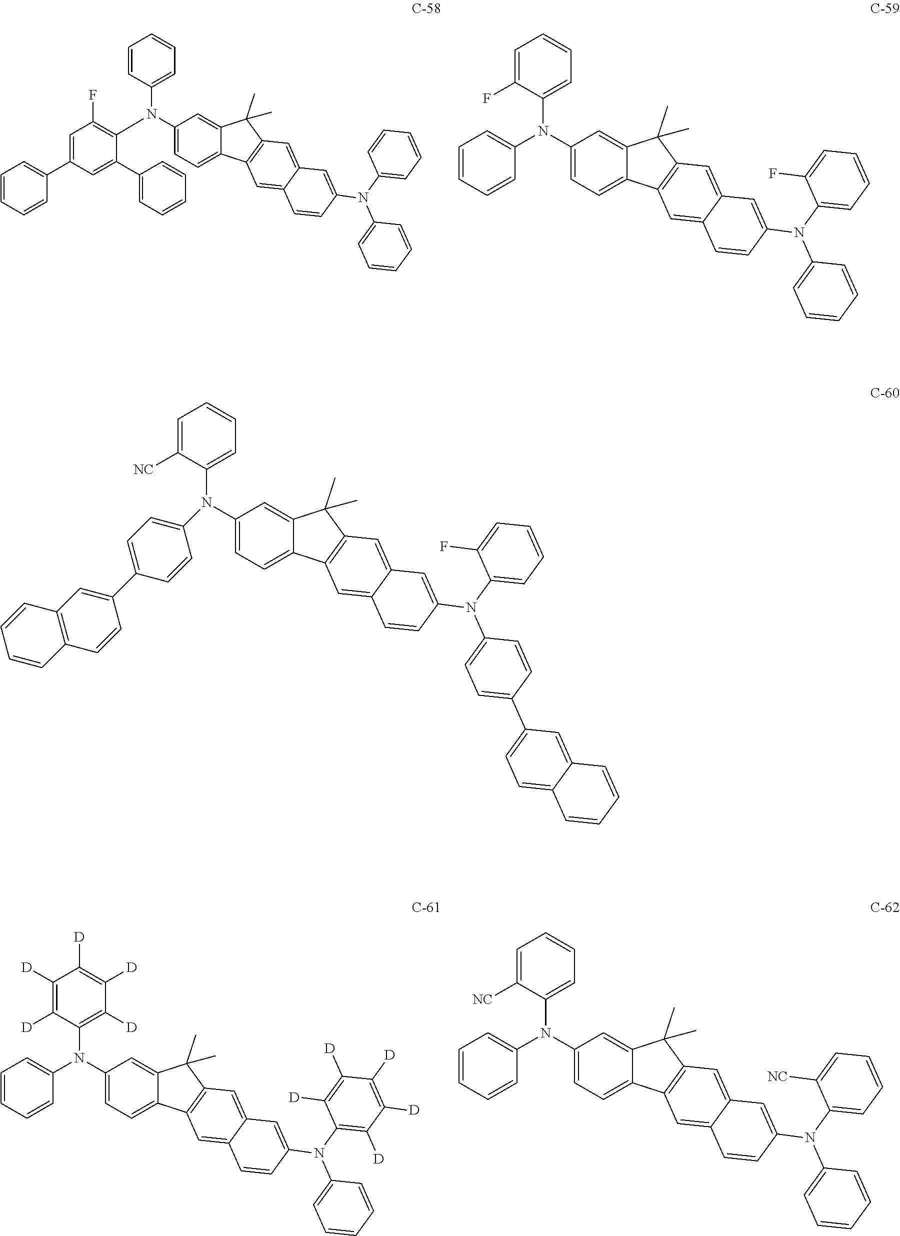

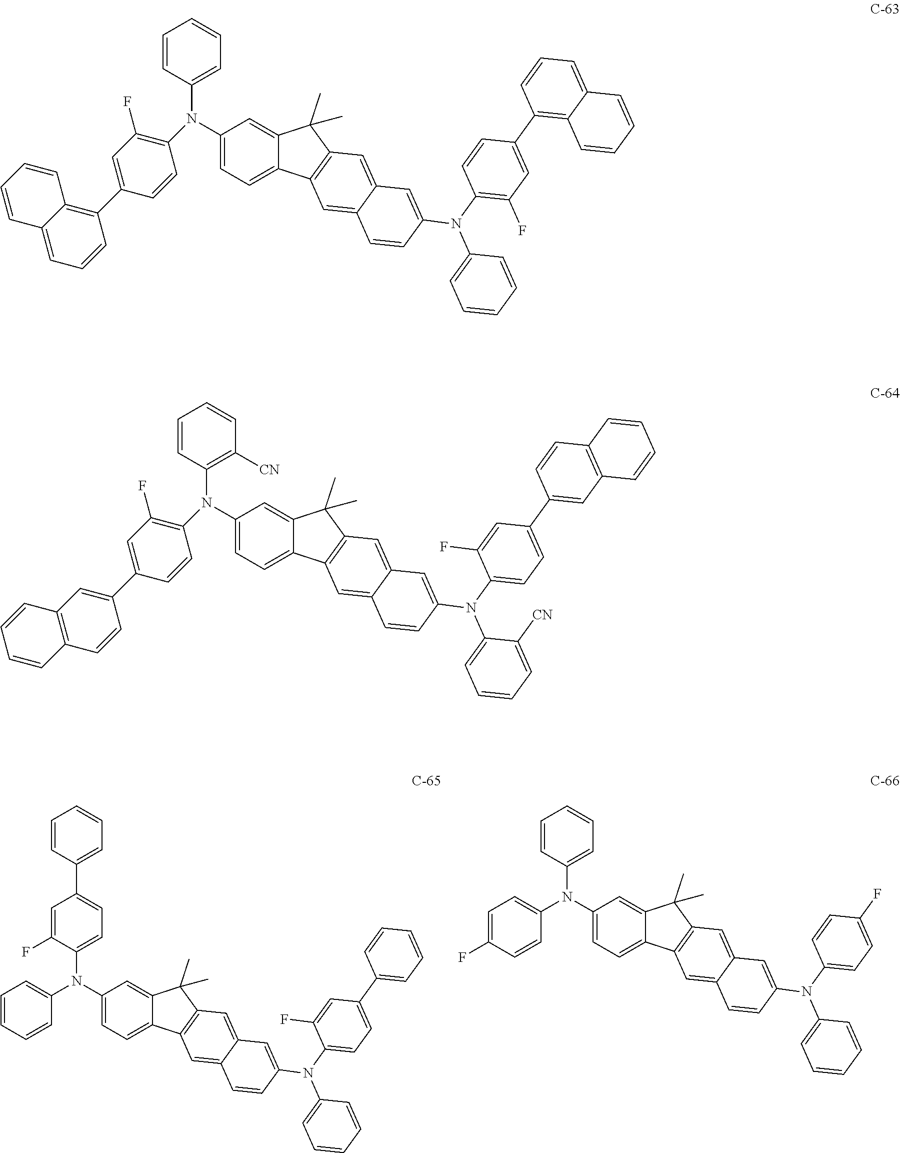

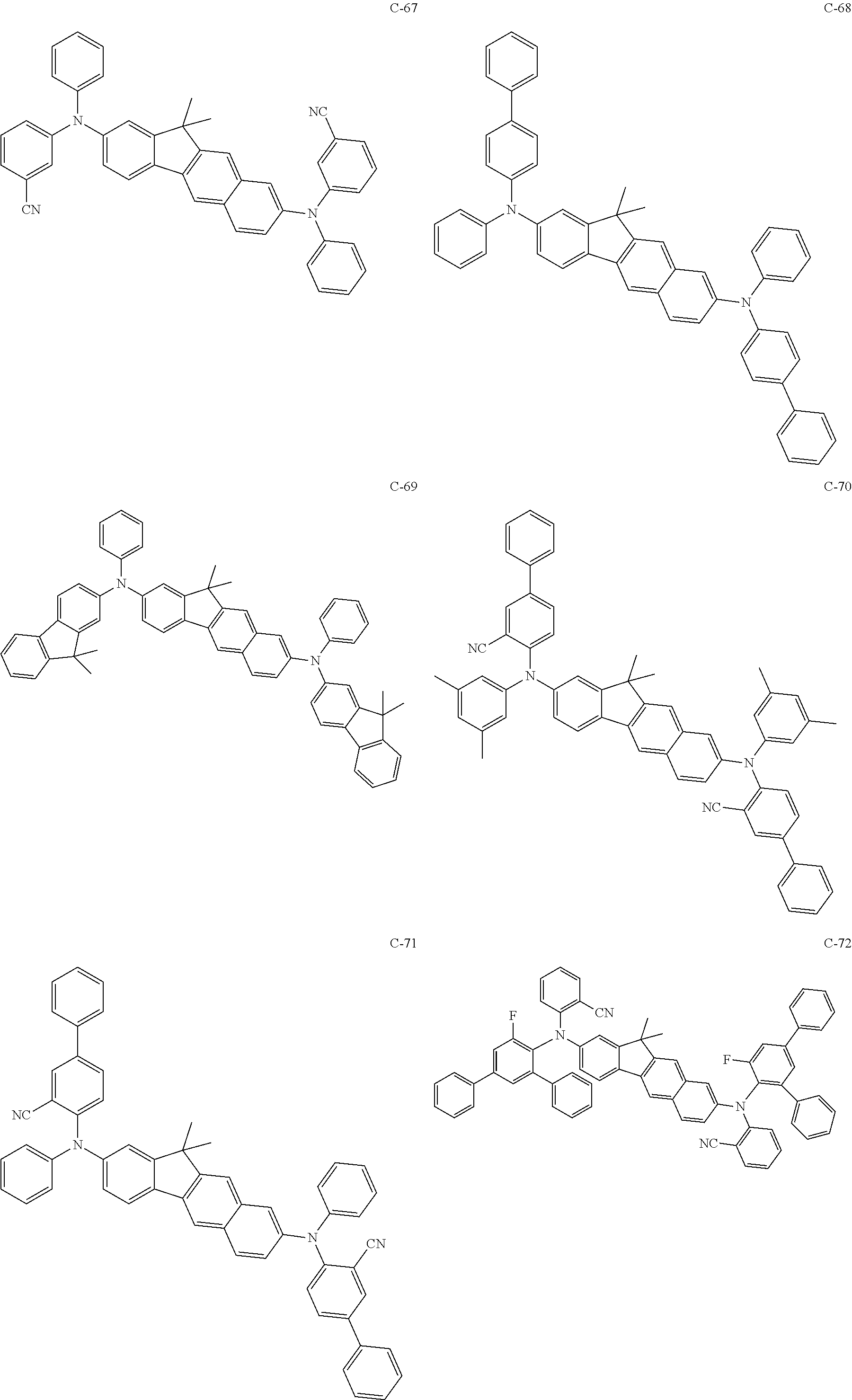

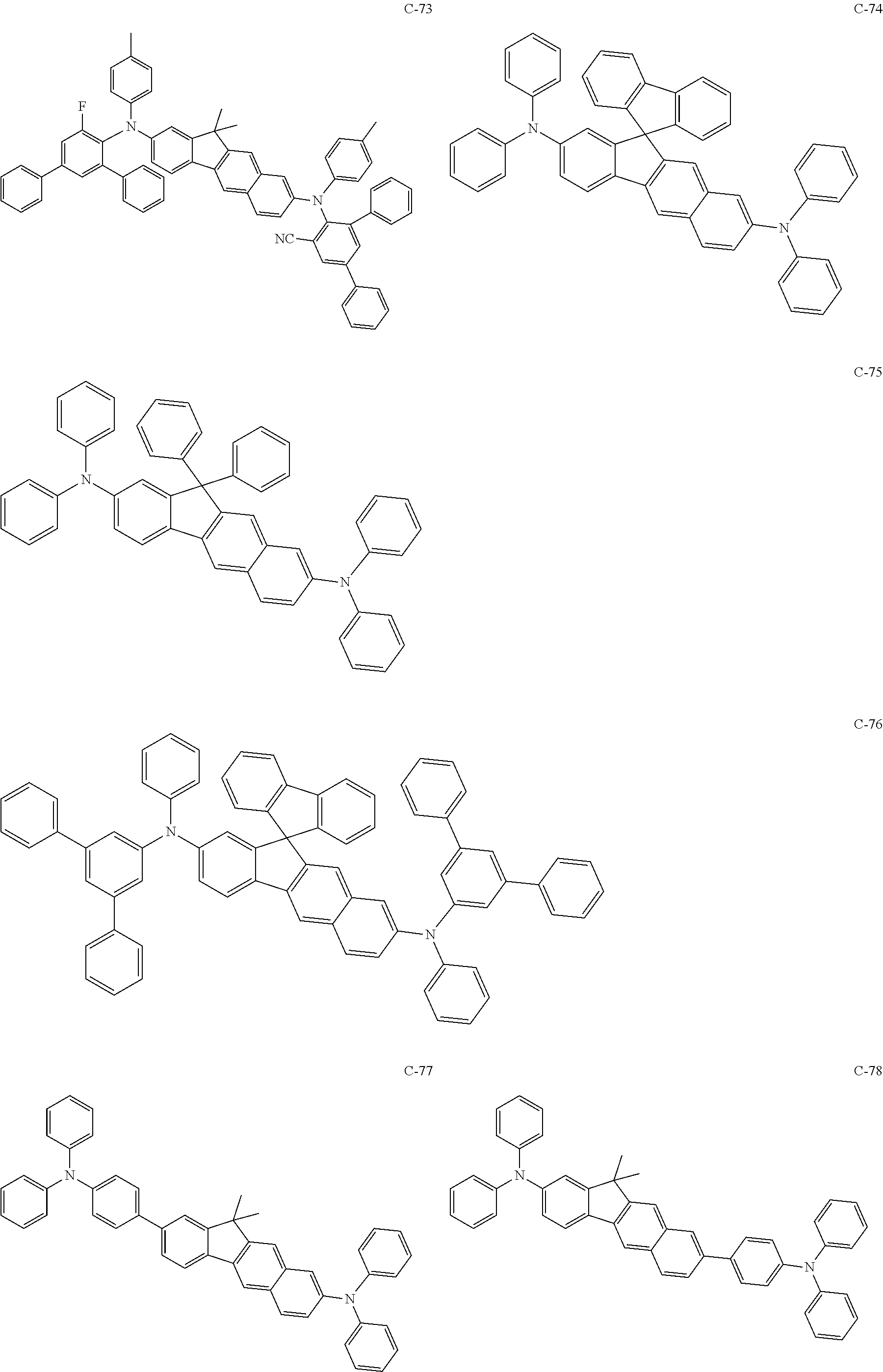

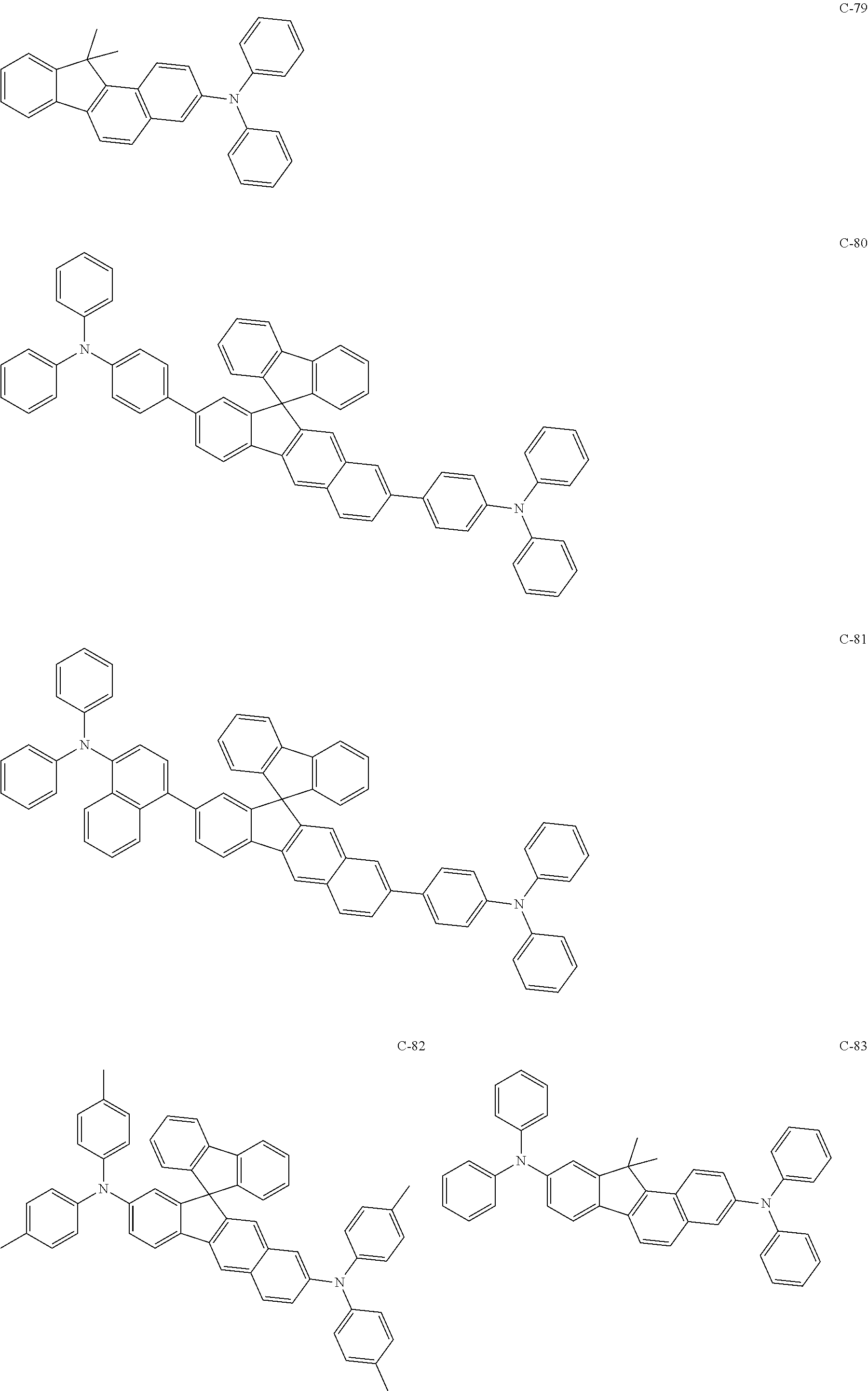

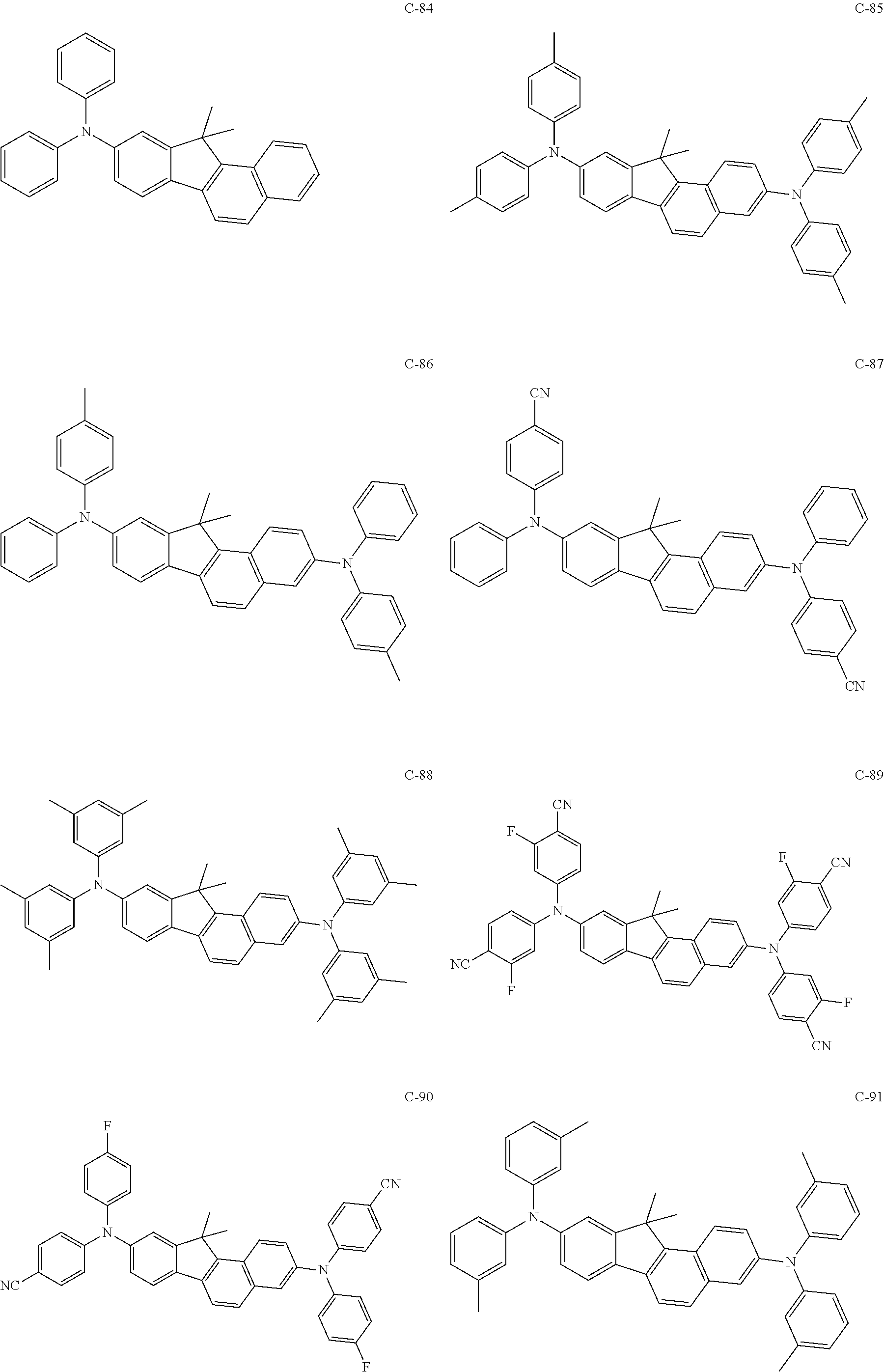

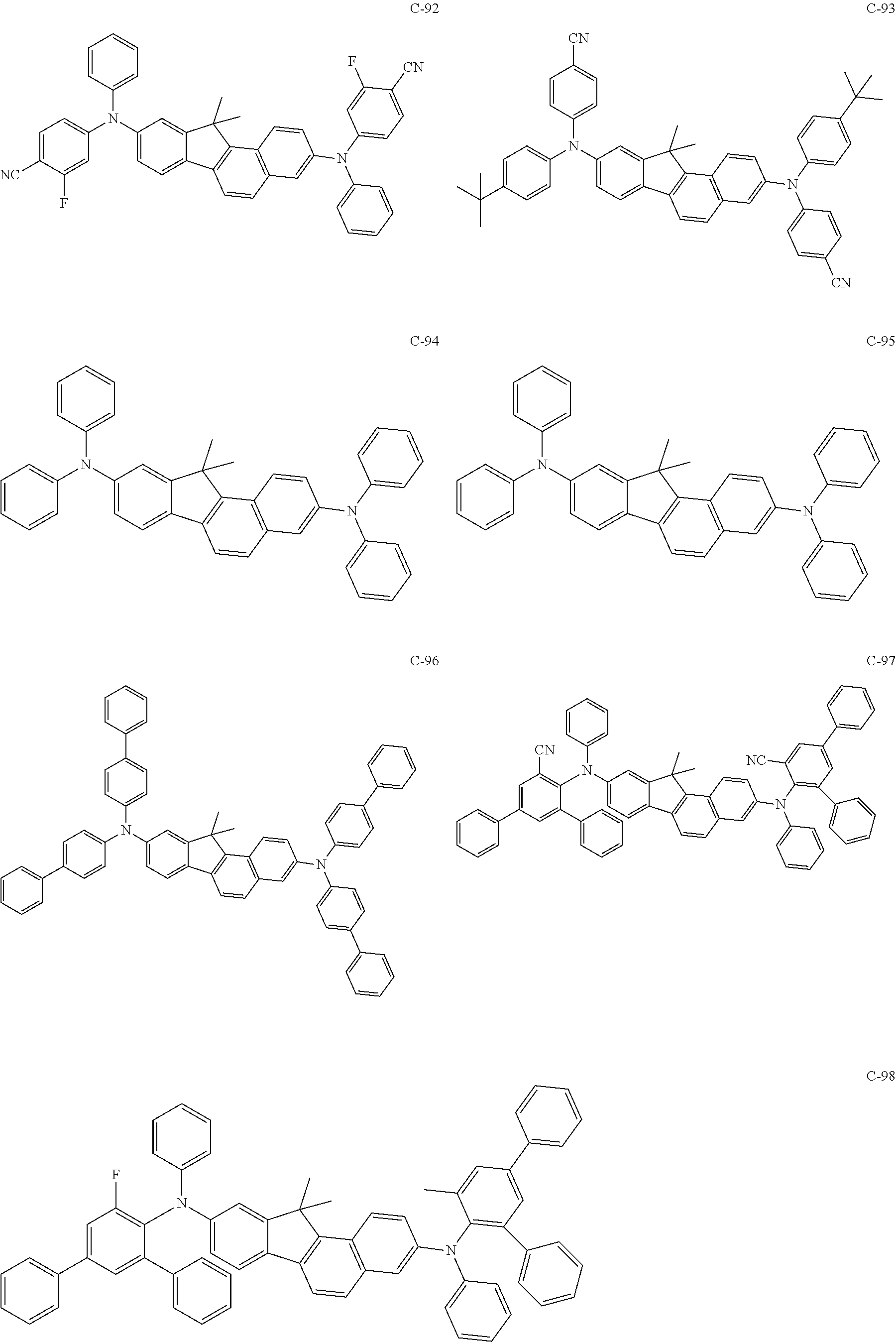

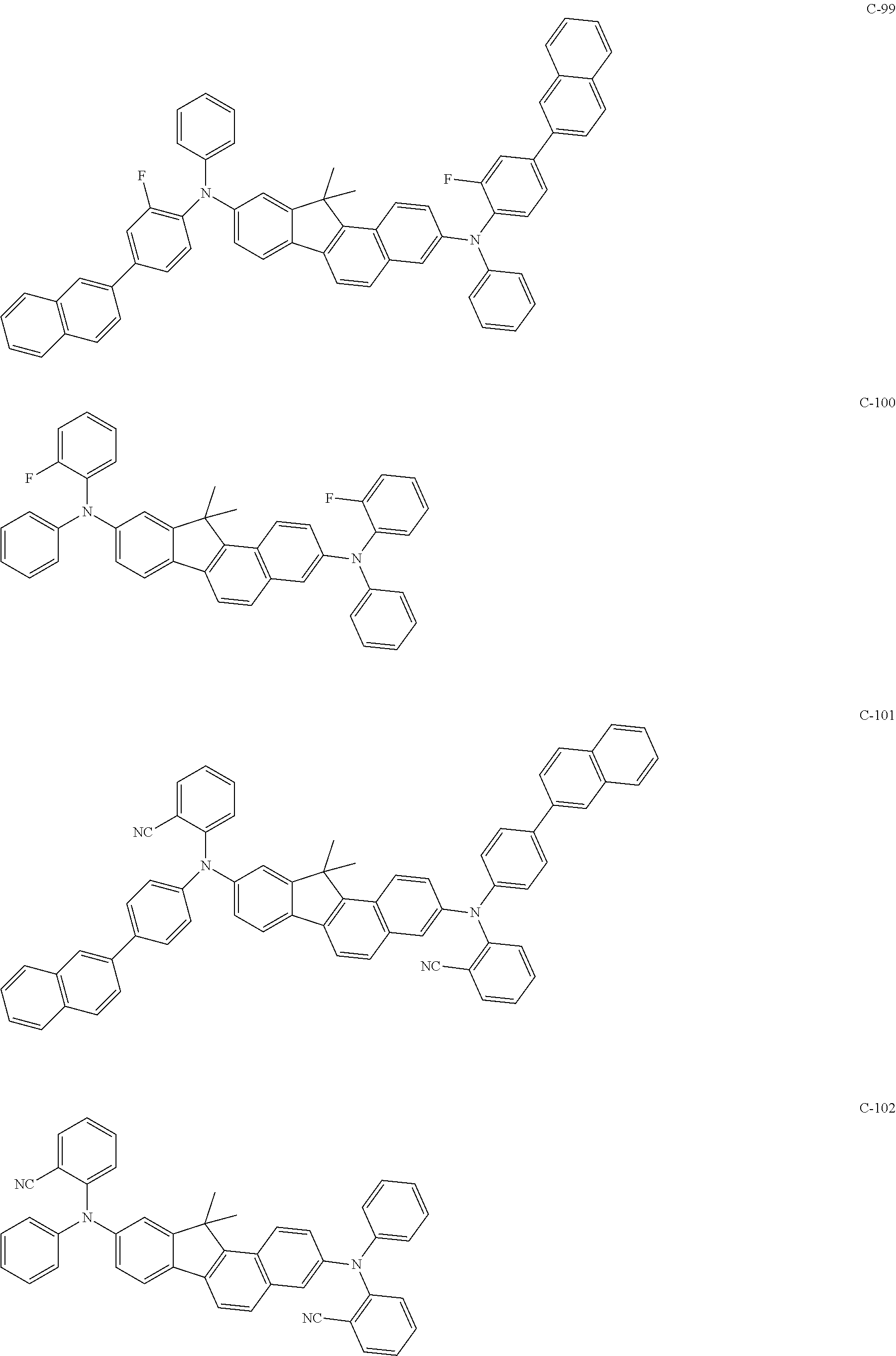 [SMILES: CC1(C)c2cc(N(c3ccccc3)c3ccc(-c4ccc5ccccc5c4)cc3F)ccc2-c2ccc3cc(N(c4ccccc4)c4ccc(-c5ccc6ccccc6c5)cc4F)ccc3c21 ; CC1(C)c2cc(N(c3ccccc3)c3ccccc3F)ccc2-c2ccc3cc(N(c4ccccc4)c4ccccc4F)ccc3c21 ; CC1(C)c2cc(N(c3ccc(-c4ccc5ccccc5c4)cc3)c3ccccc3C#N)ccc2-c2ccc3cc(N(c4ccc(-c5ccc6ccccc6c5)cc4)c4ccccc4C#N)ccc3c21 ; CC1(C)c2cc(N(c3ccccc3)c3ccccc3C#N)ccc2-c2ccc3cc(N(c4ccccc4)c4ccccc4C#N)ccc3c21]

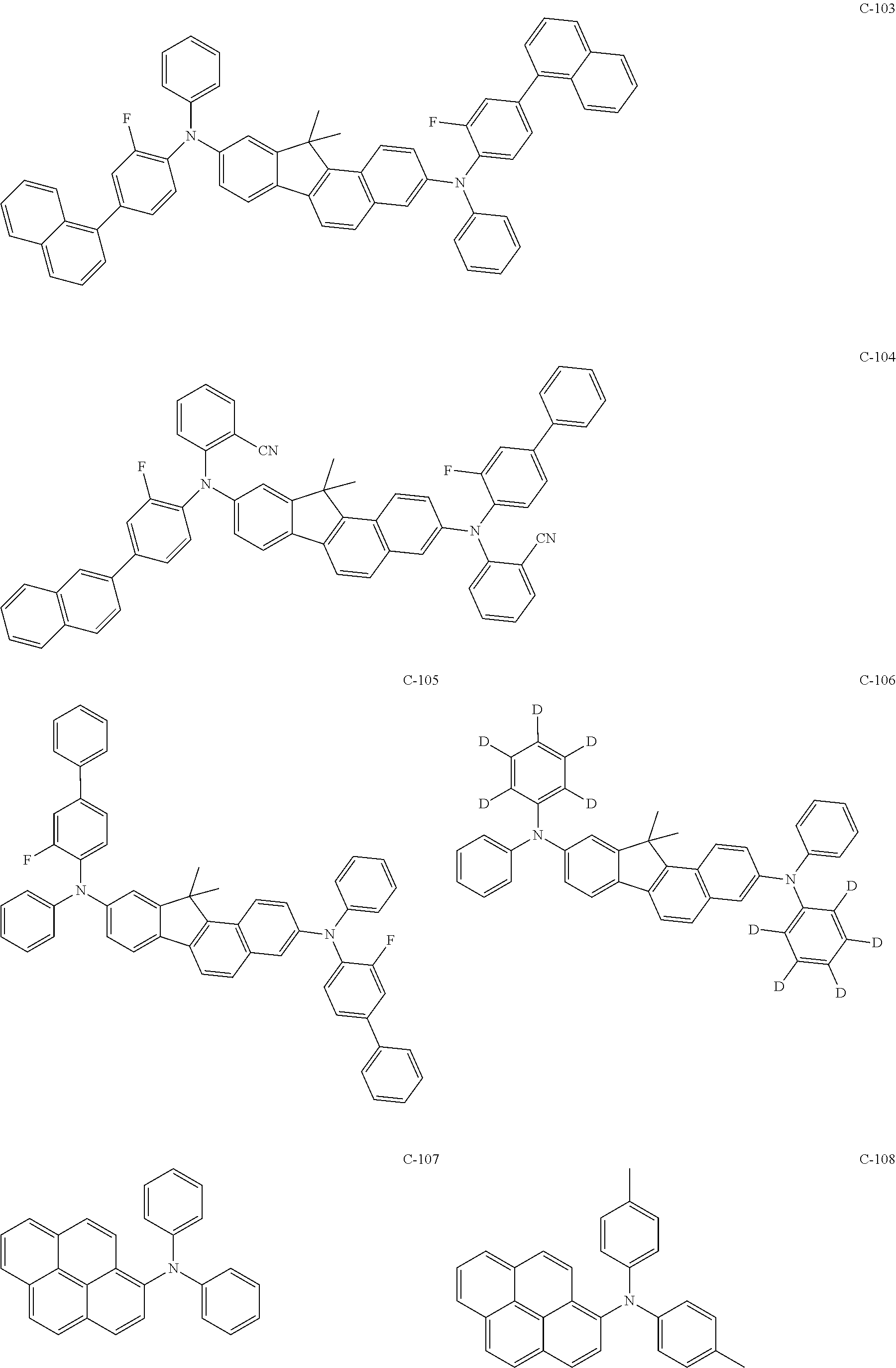

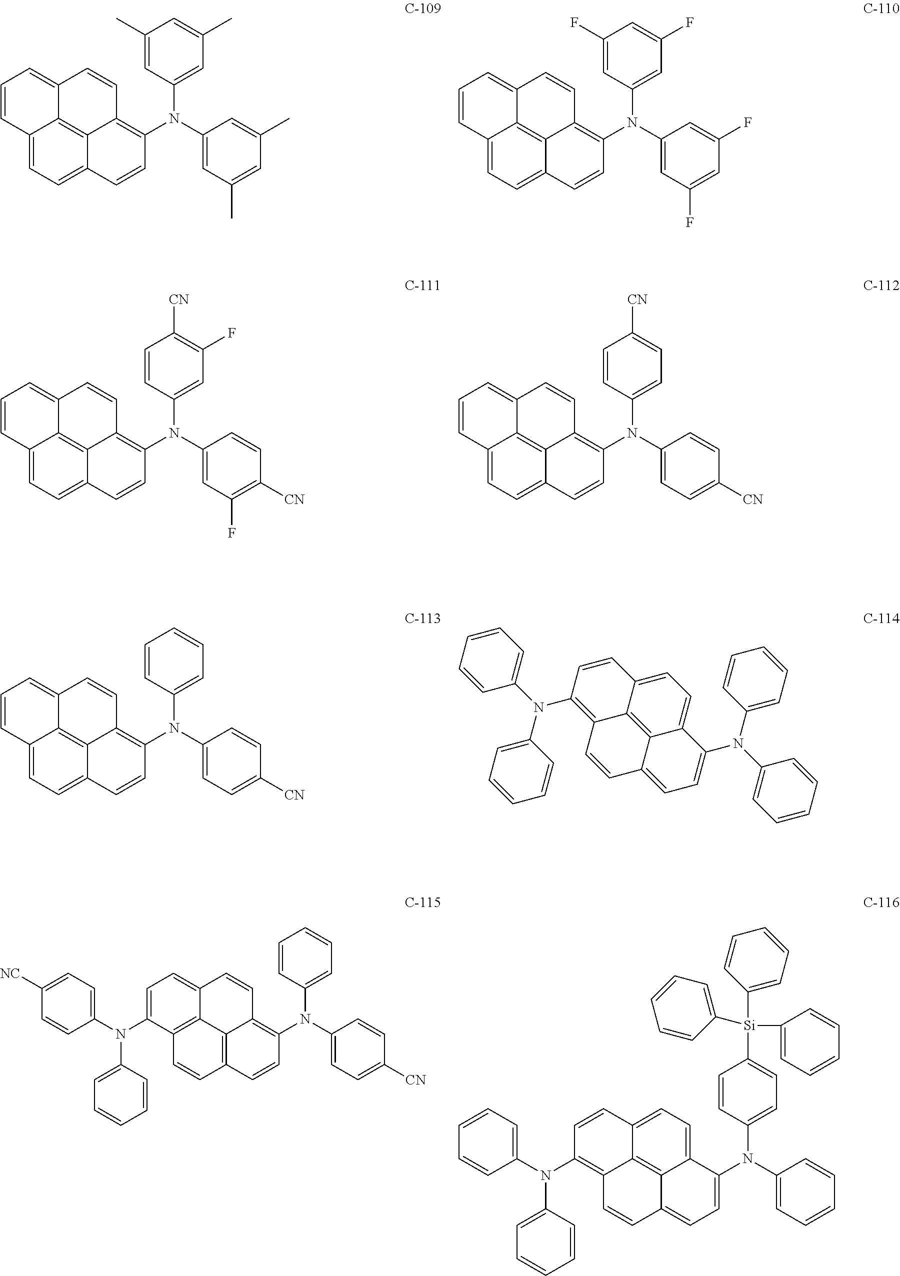

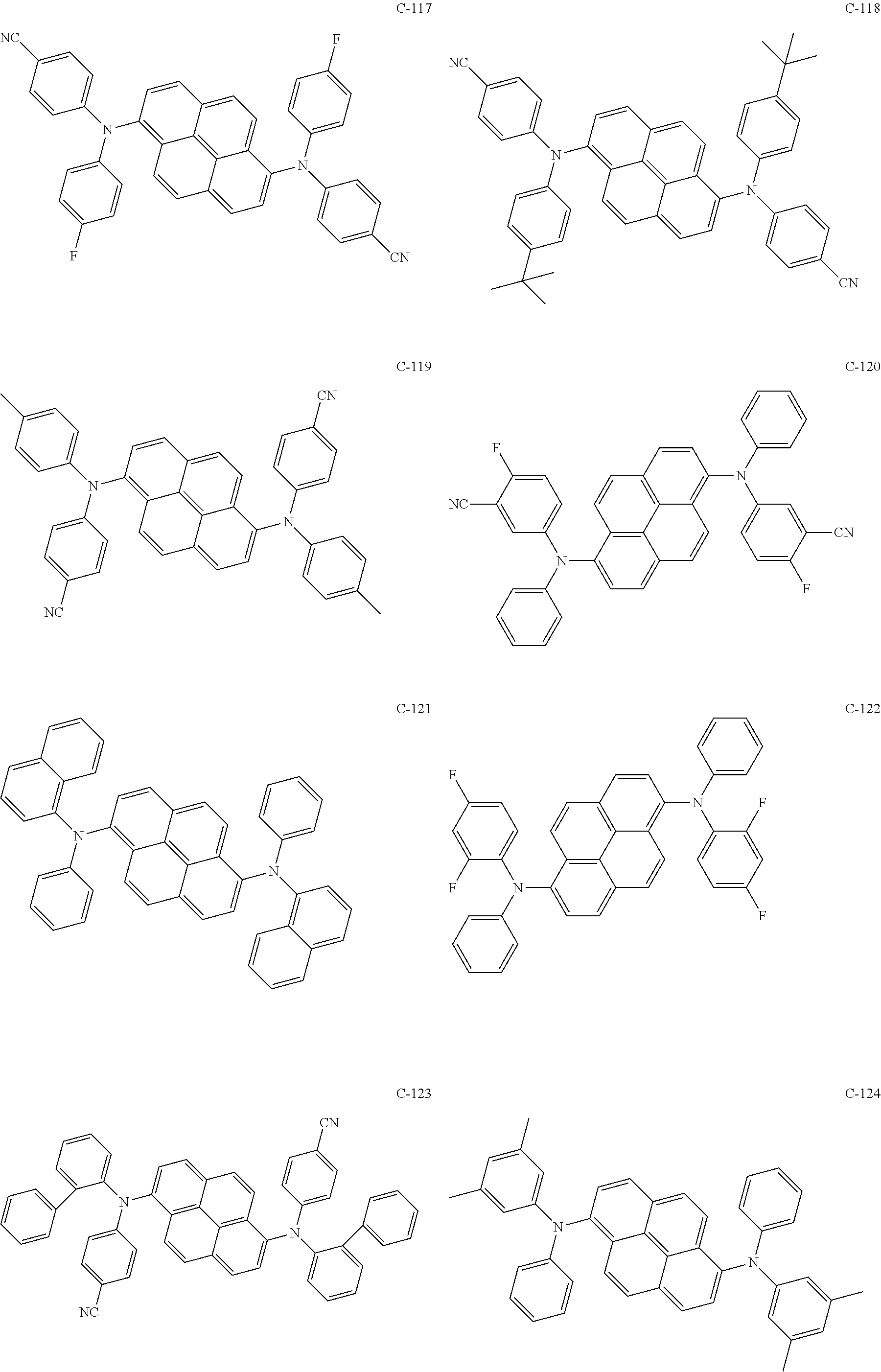

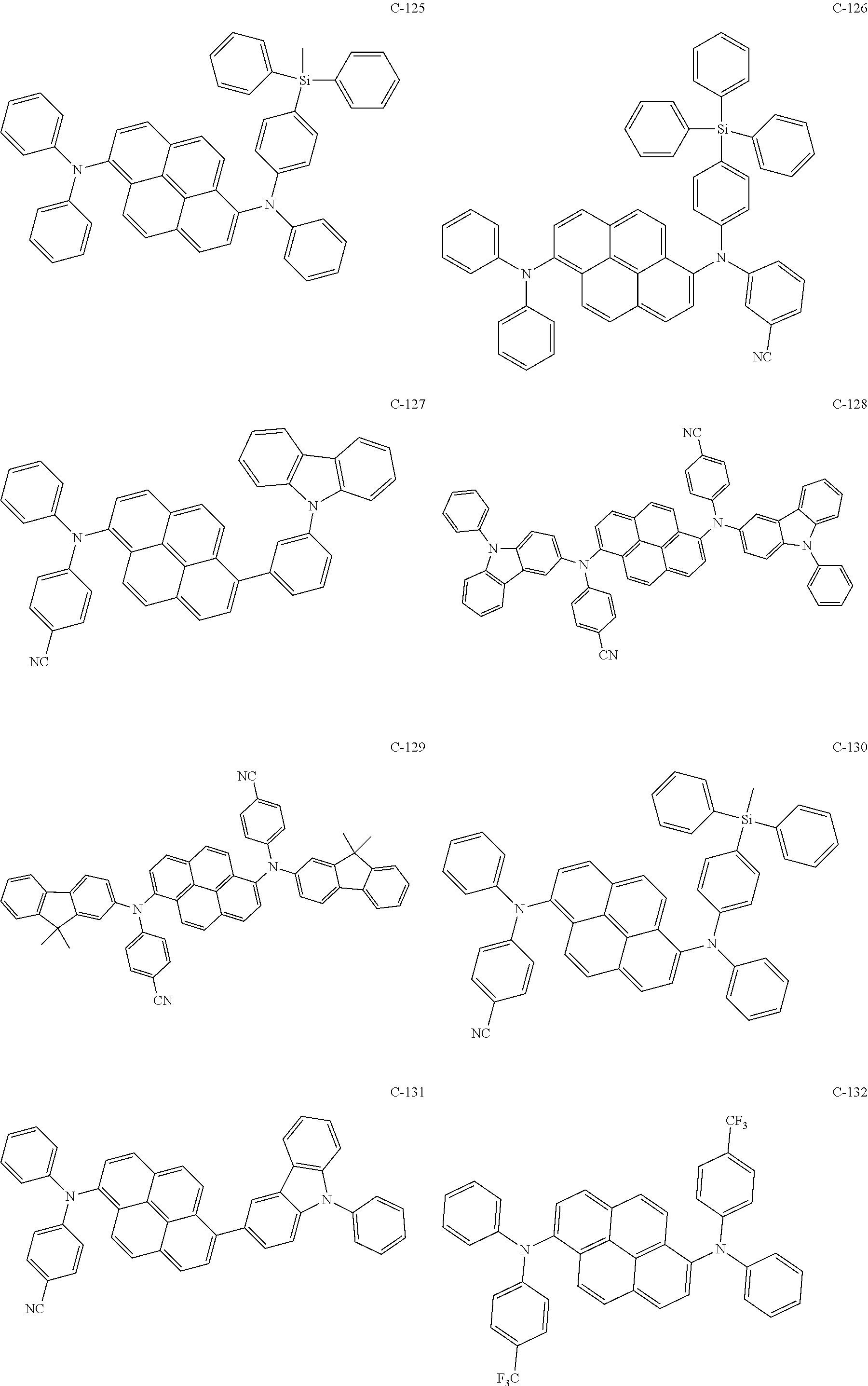

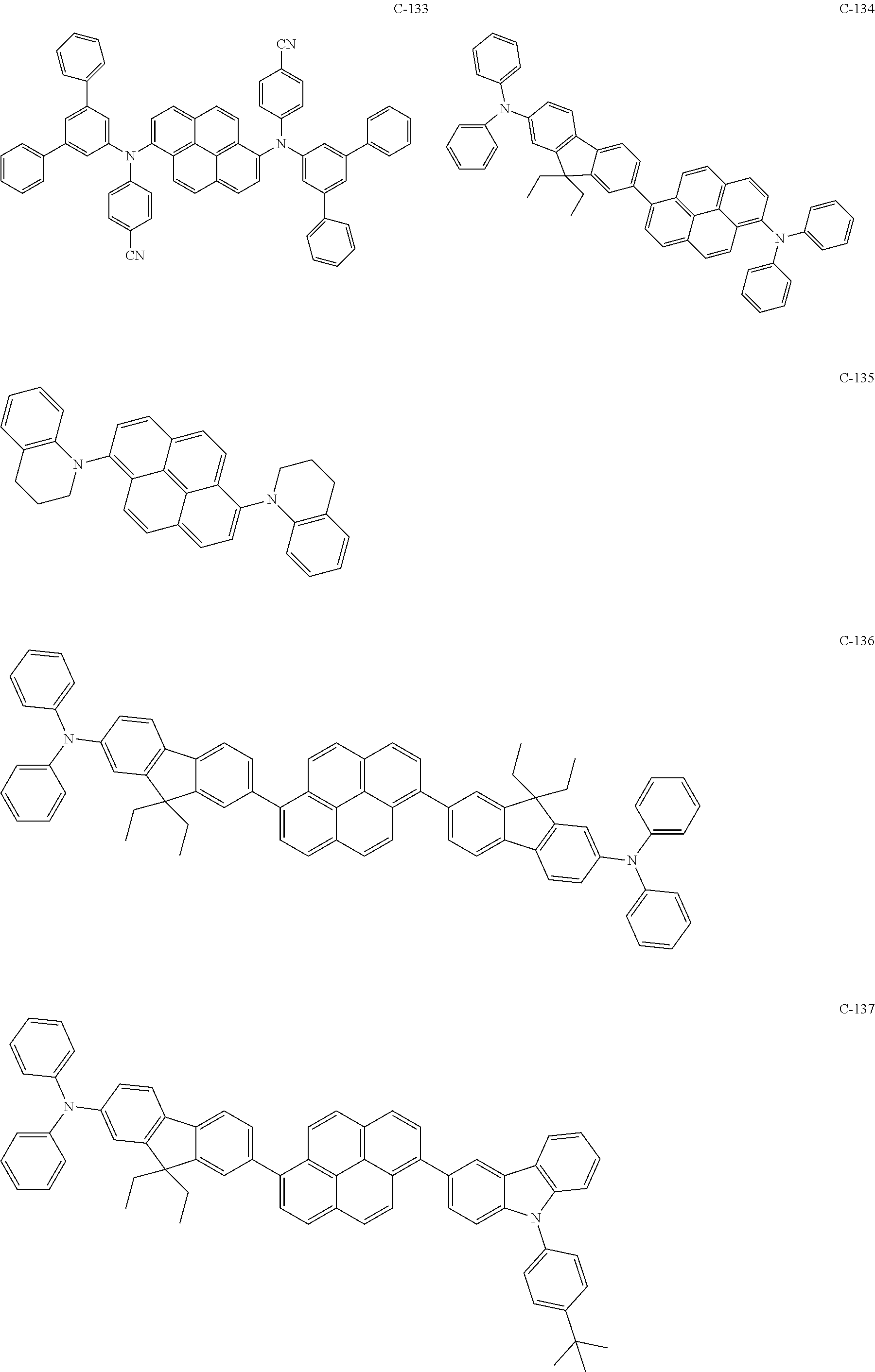

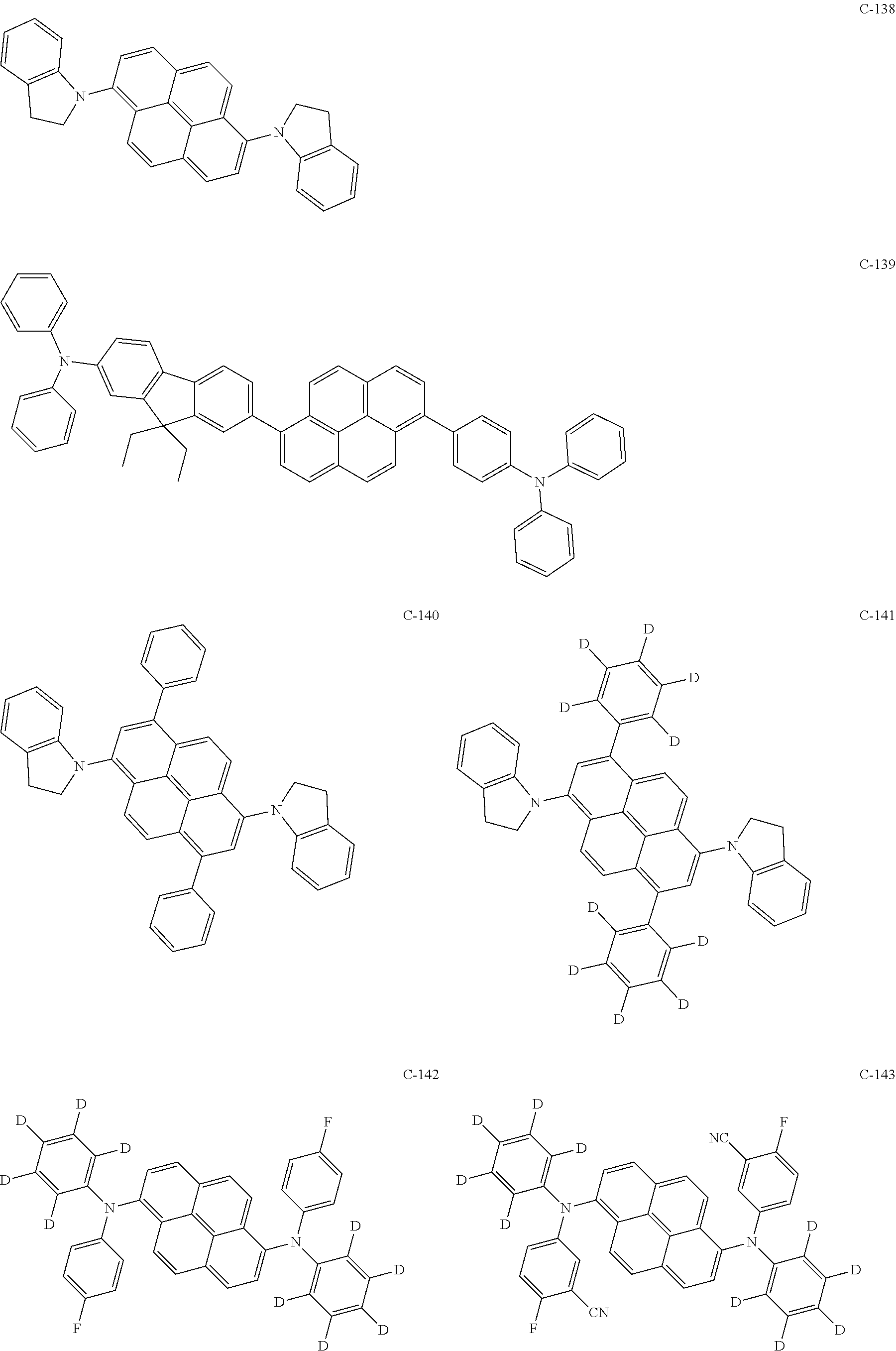

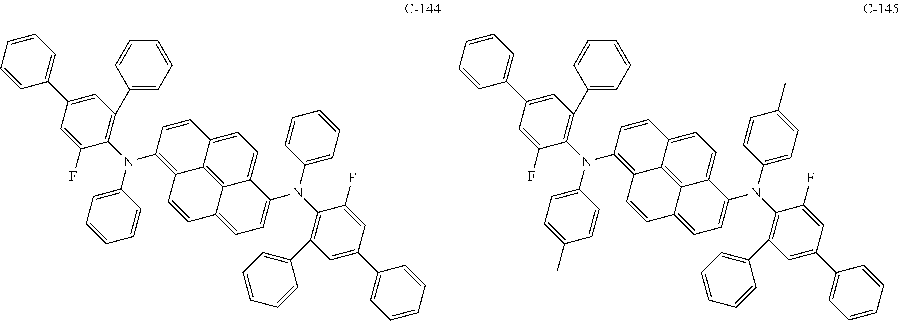 [SMILES: Fc1cc(-c2ccccc2)cc(-c2ccccc2)c1N(c1ccccc1)c1ccc2ccc3c(N(c4ccccc4)c4c(F)cc(-c5ccccc5)cc4-c4ccccc4)ccc4ccc1c2c43 ; Cc1ccc(N(c2c(F)cc(-c3ccccc3)cc2-c2ccccc2)c2ccc3ccc4c(N(c5ccc(C)cc5)c5c(F)cc(-c6ccccc6)cc5-c5ccccc5)ccc5ccc2c3c54)cc1]

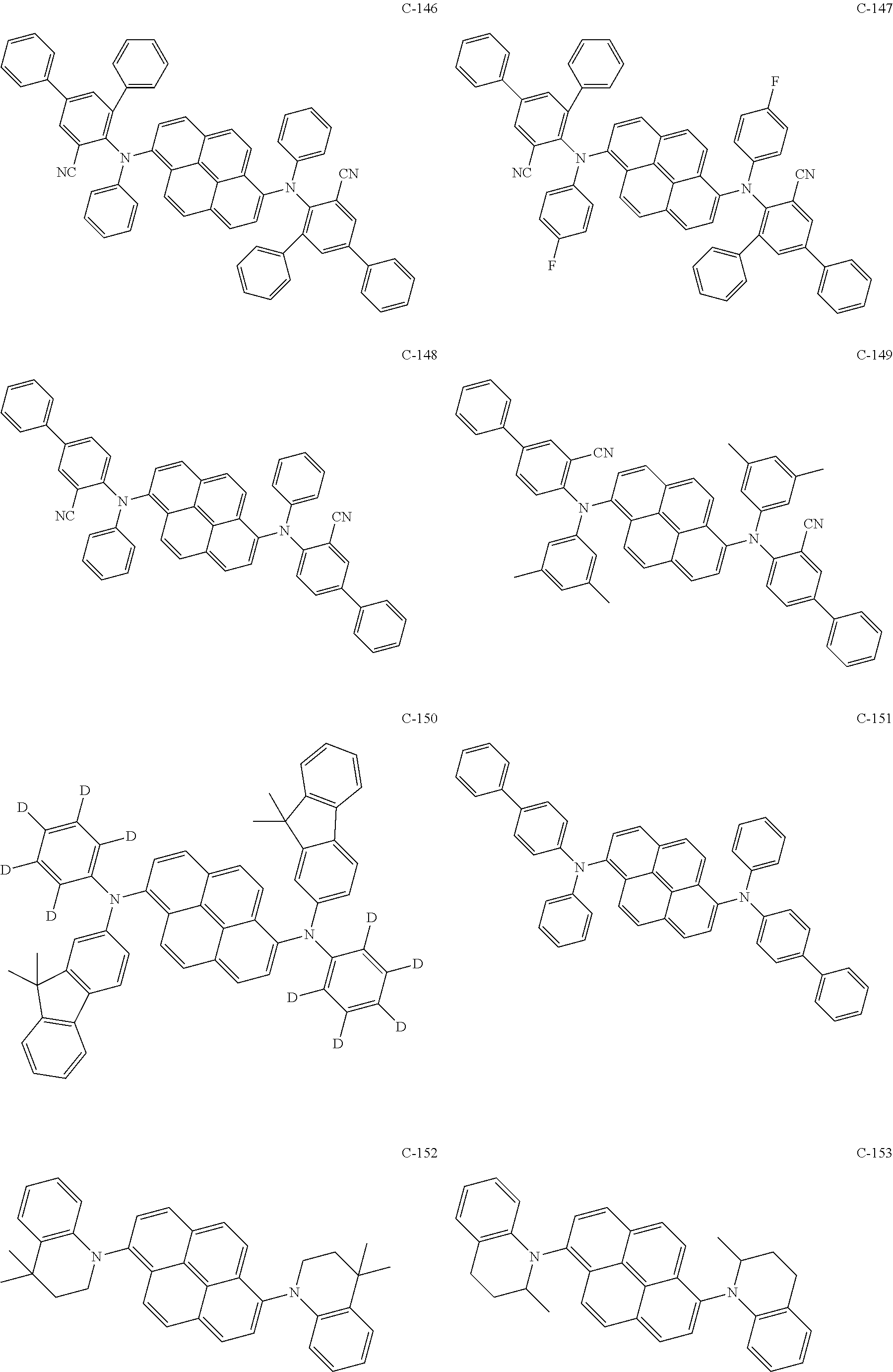

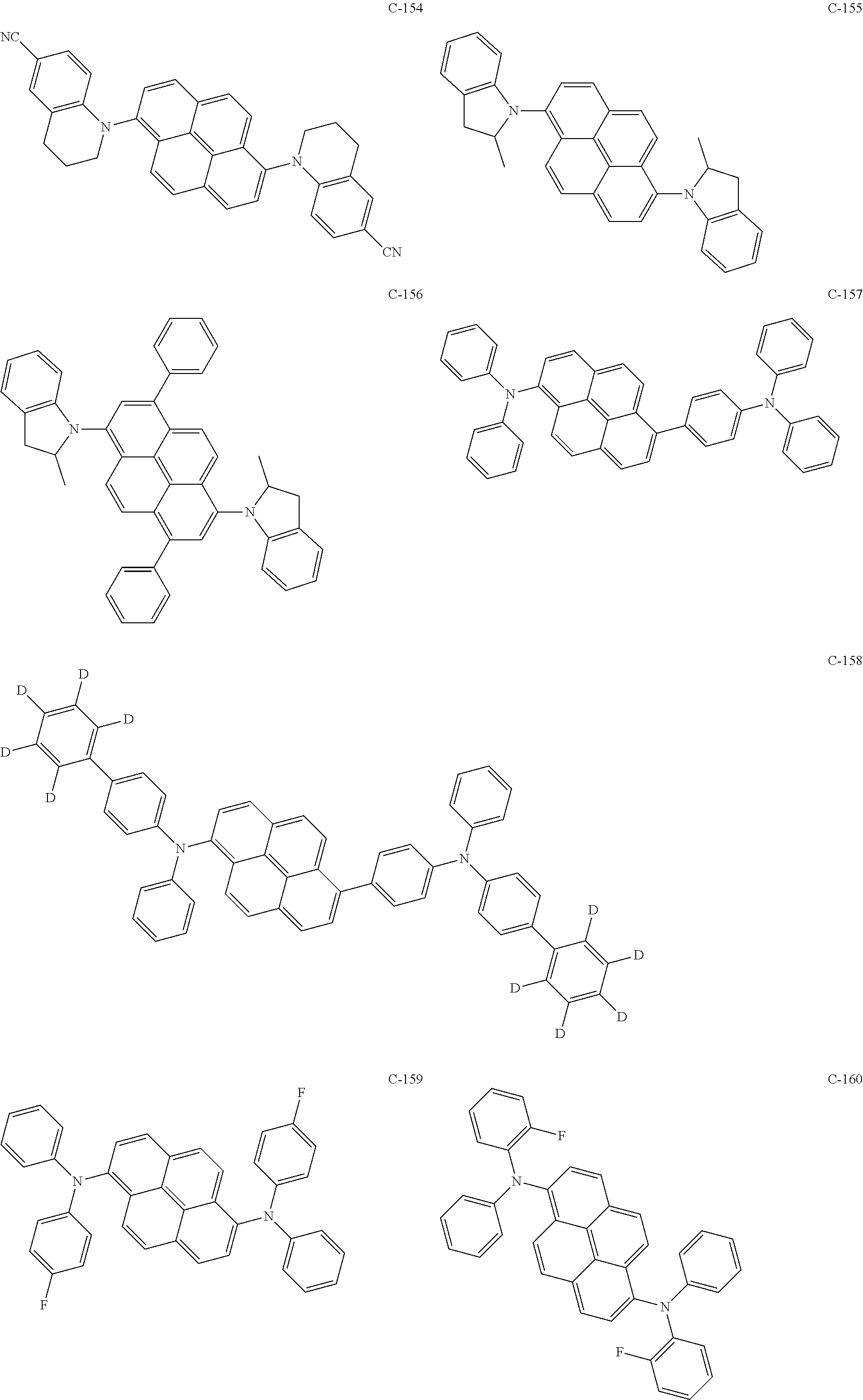

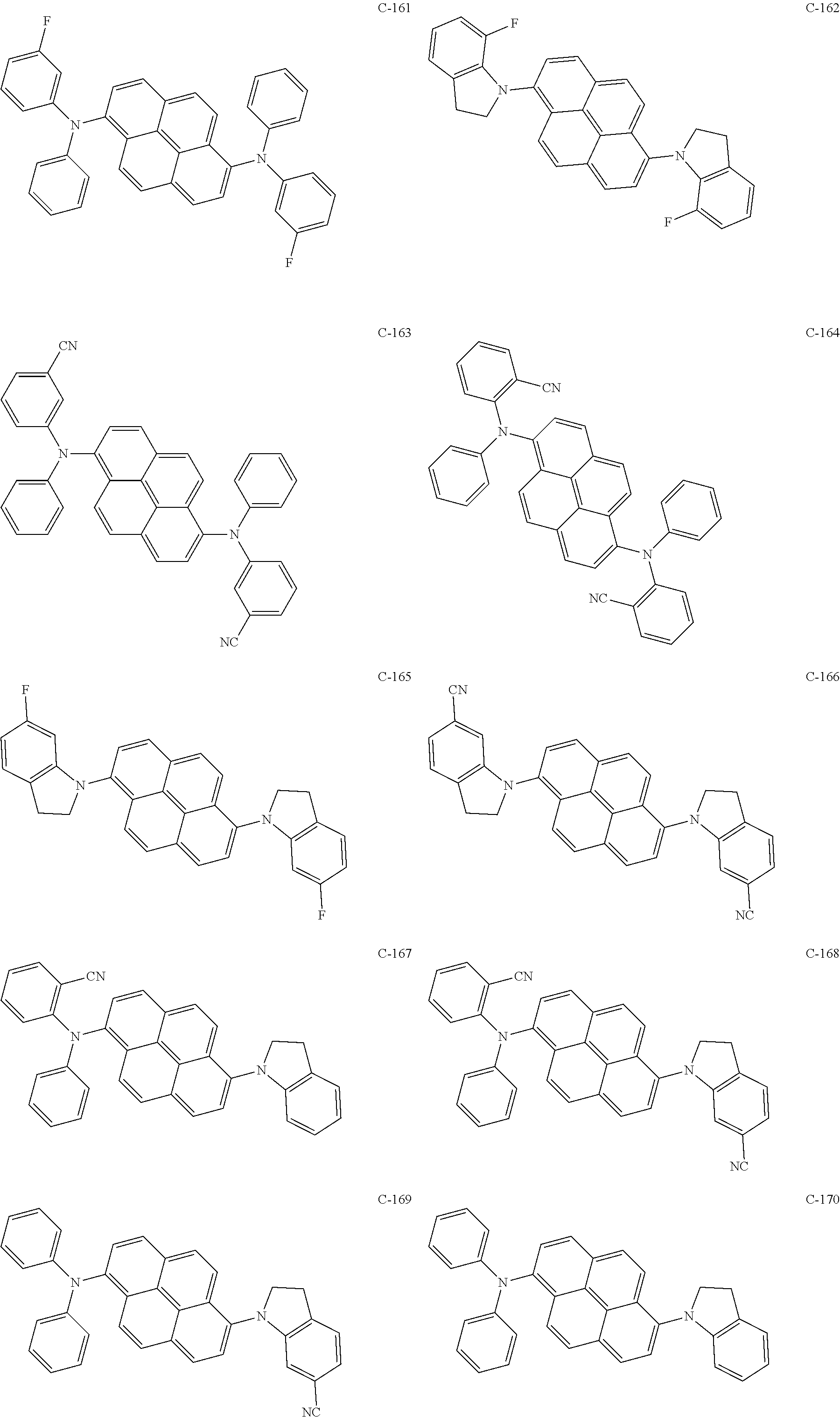

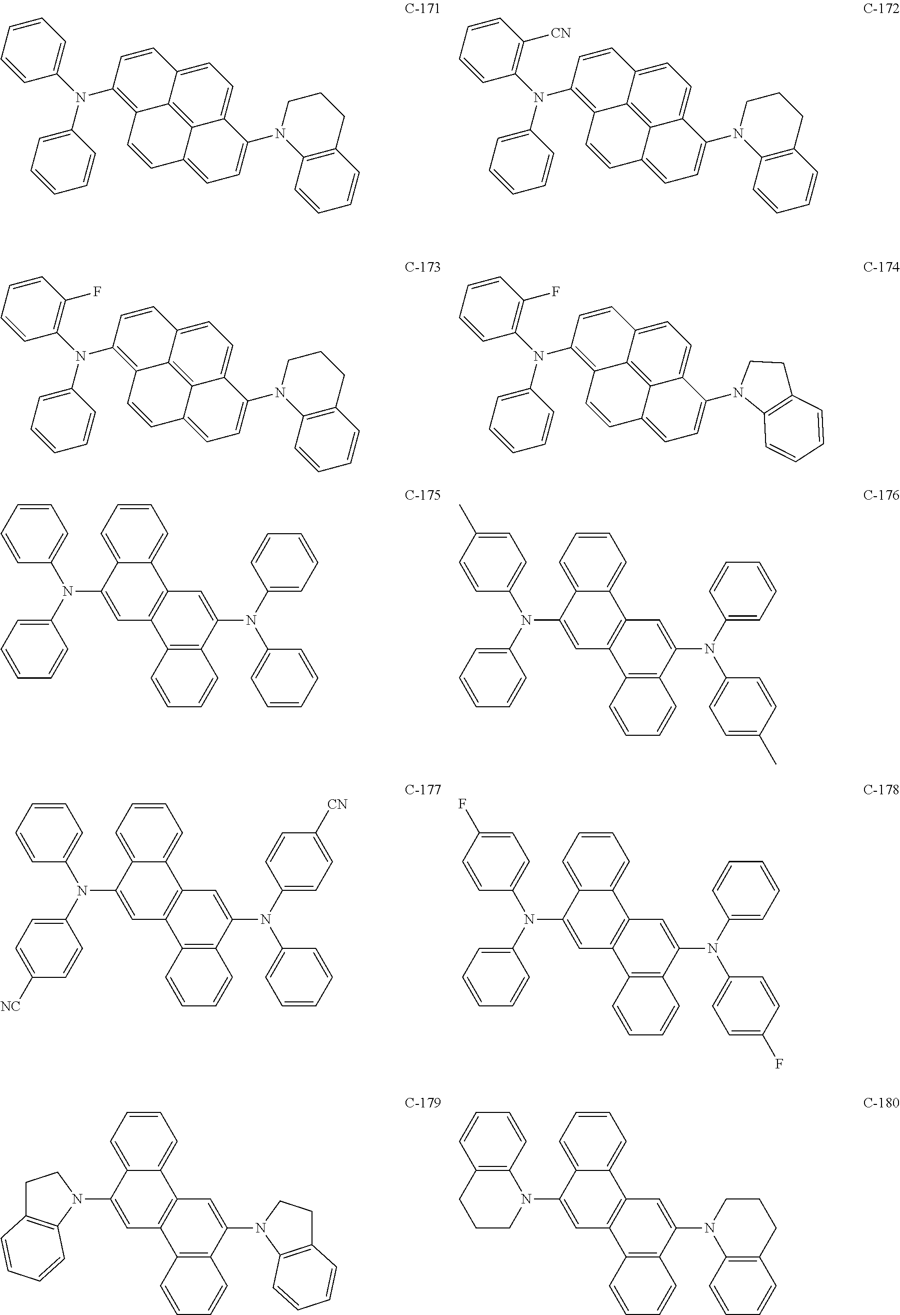

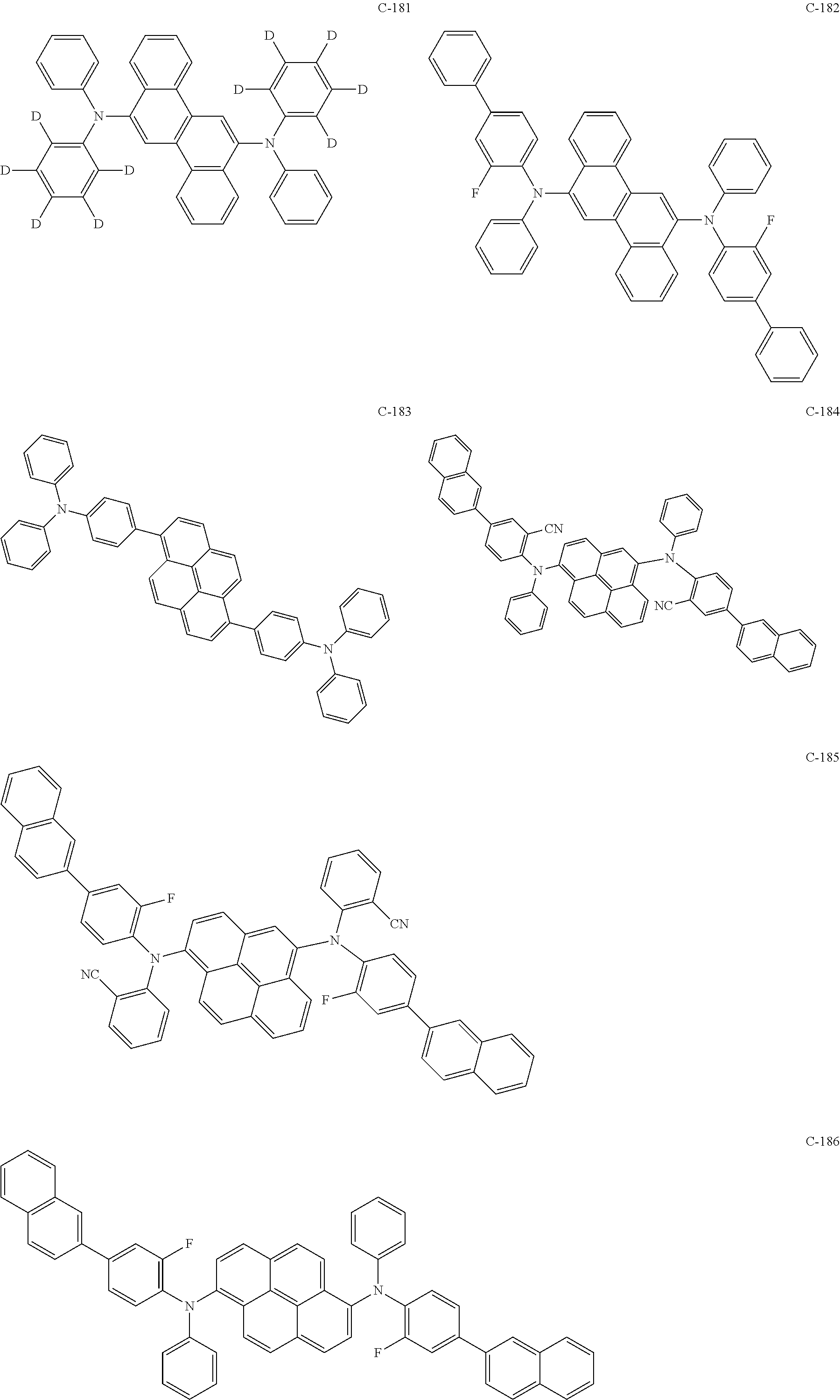

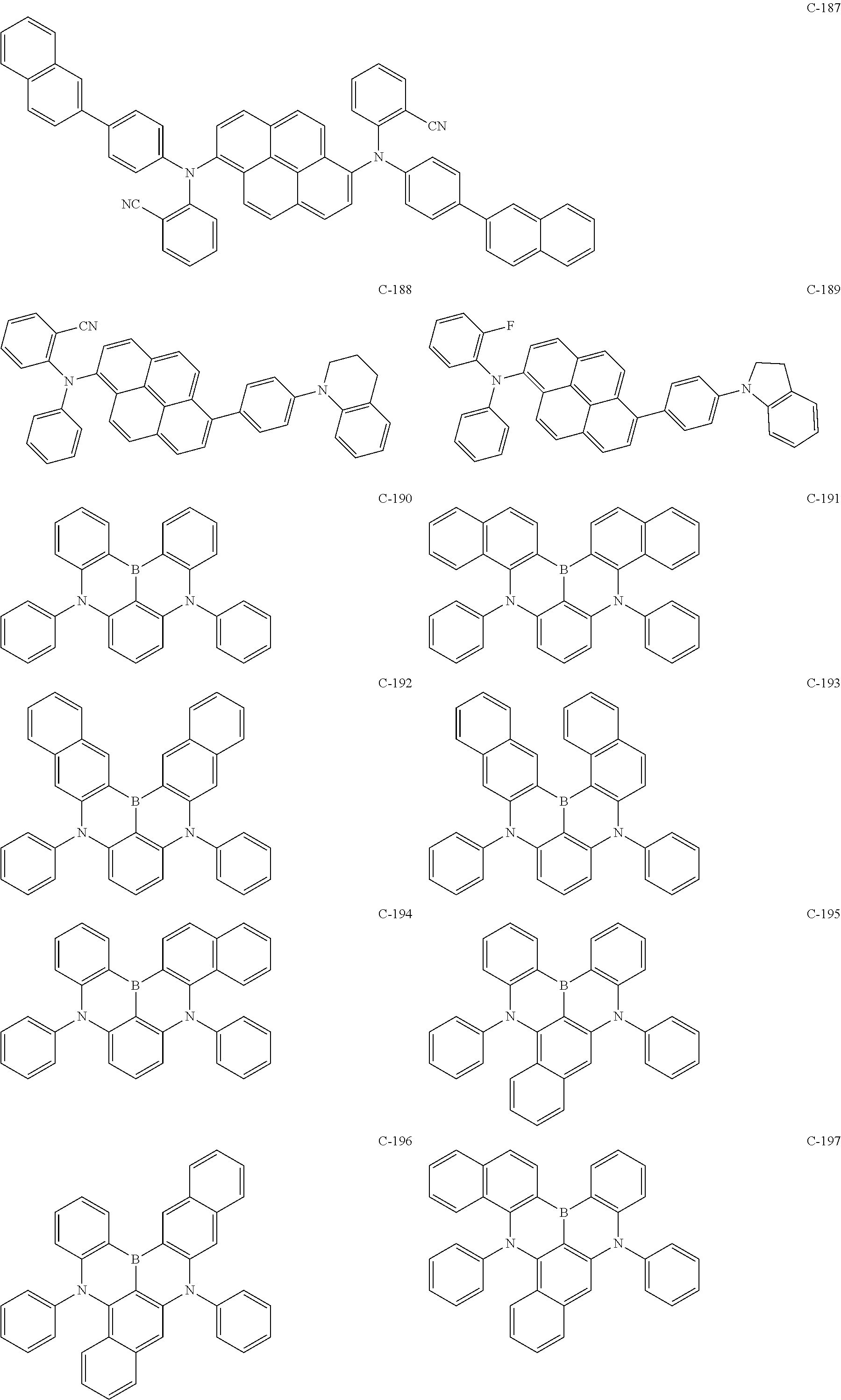

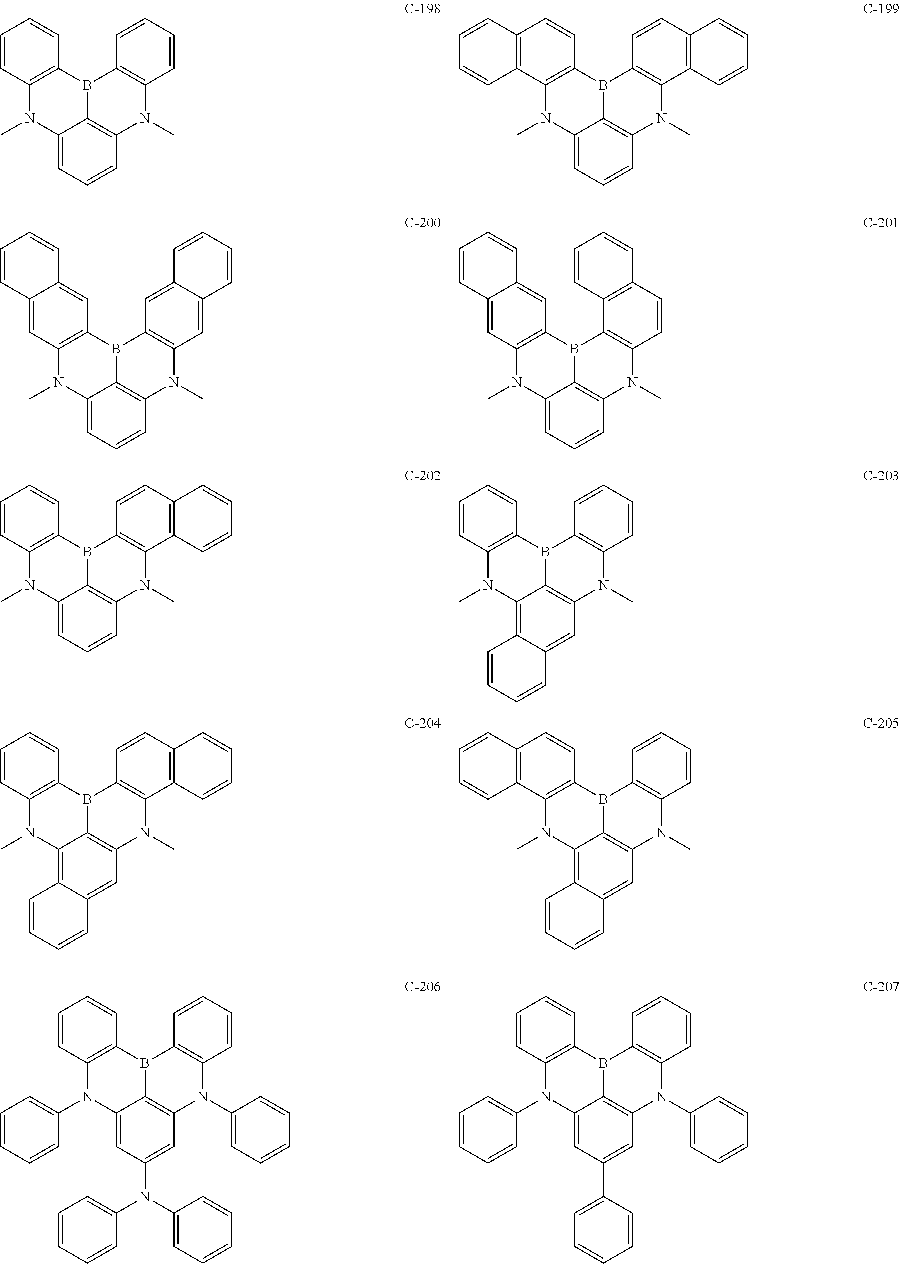

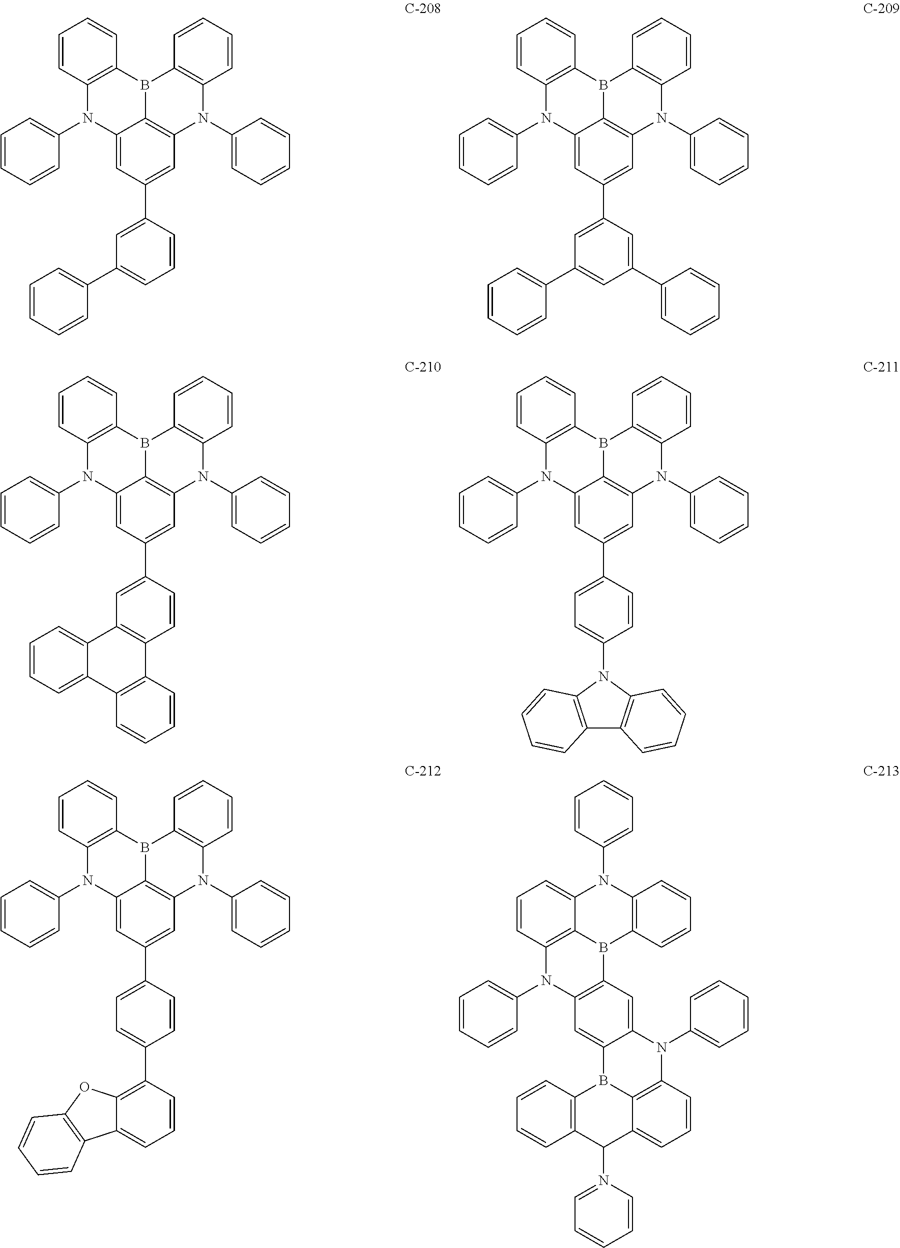

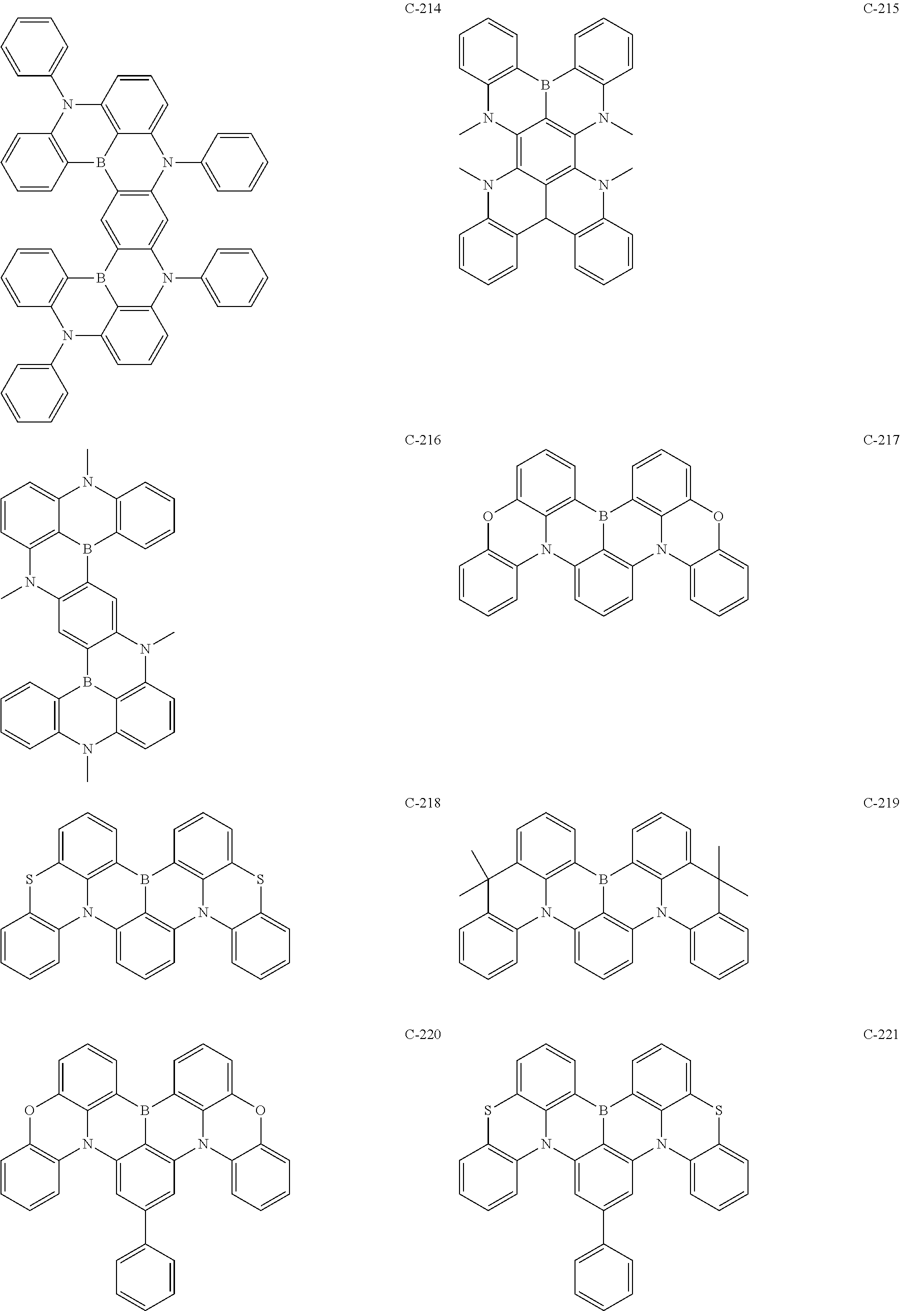

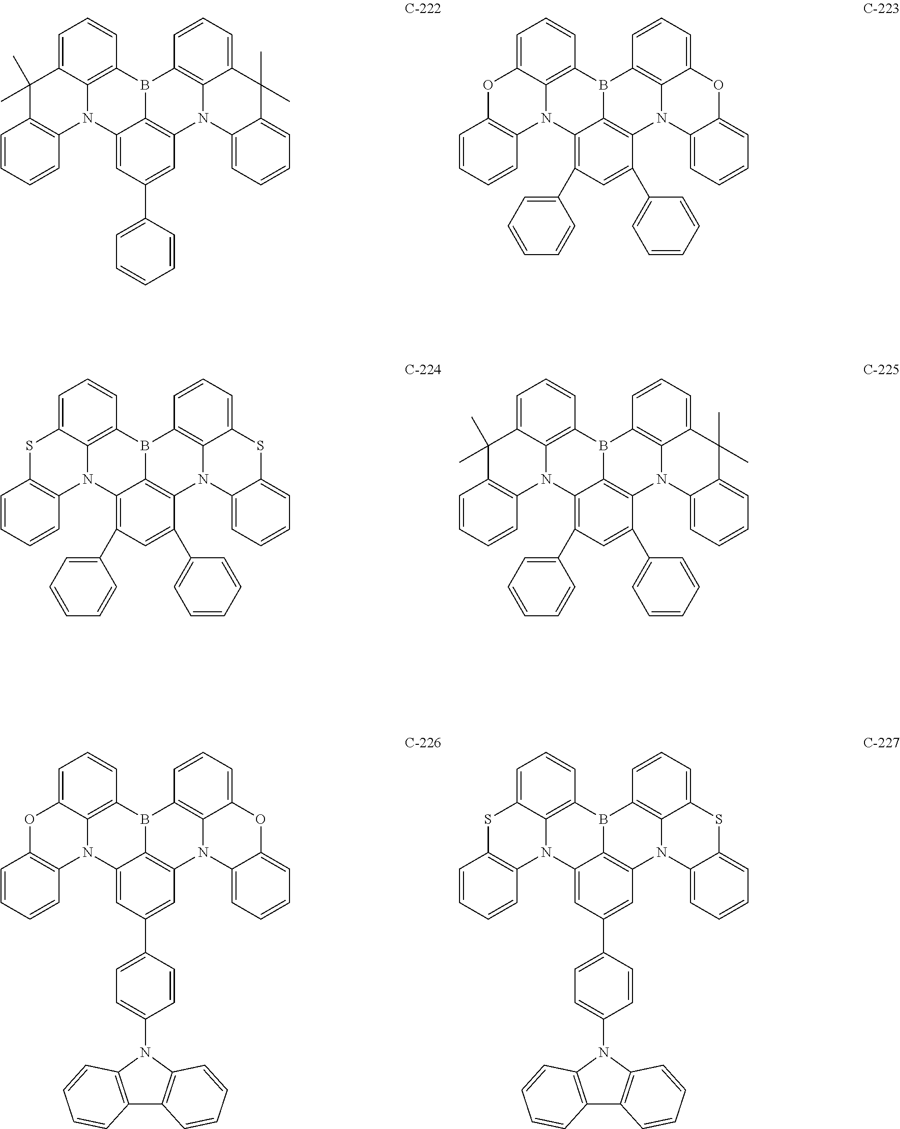

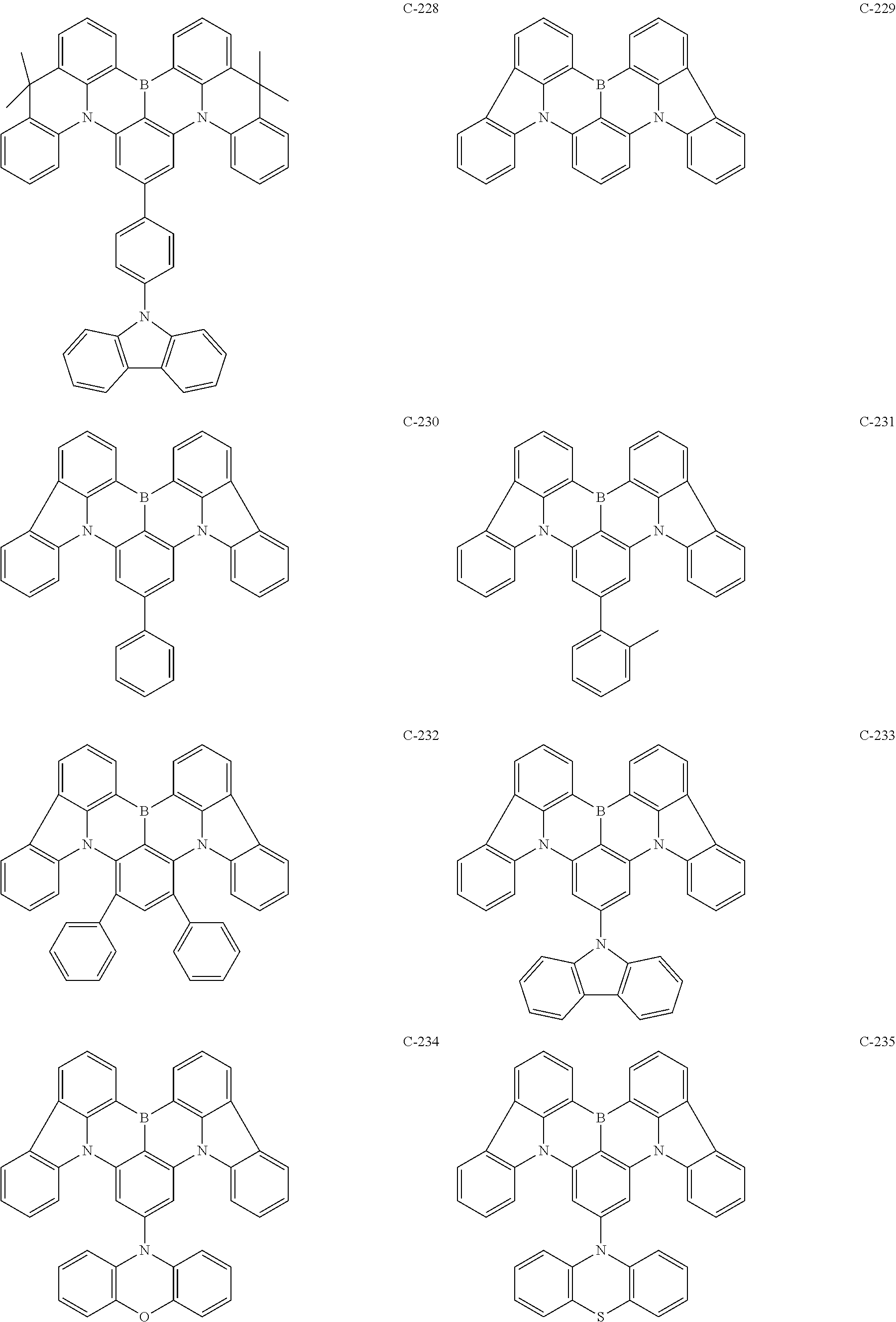

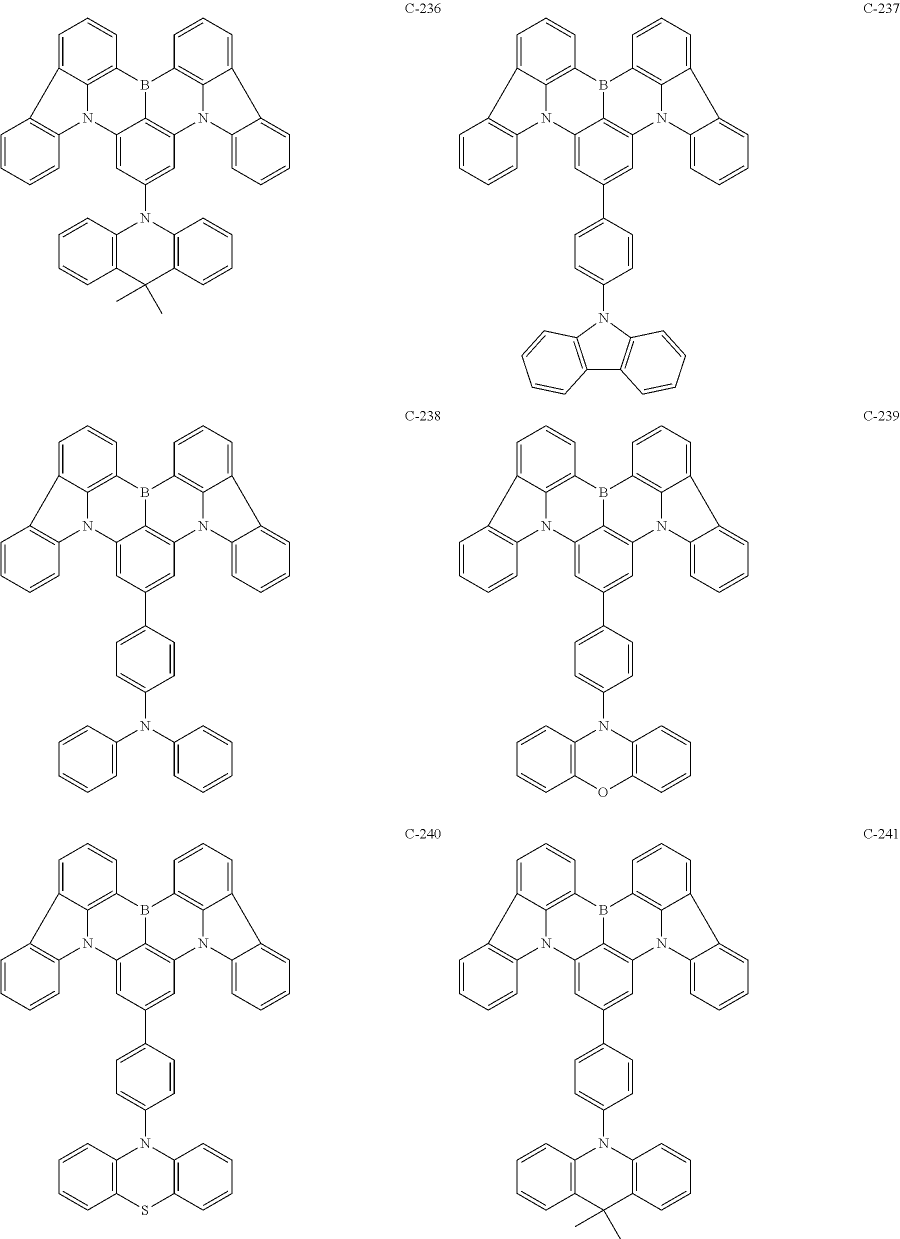

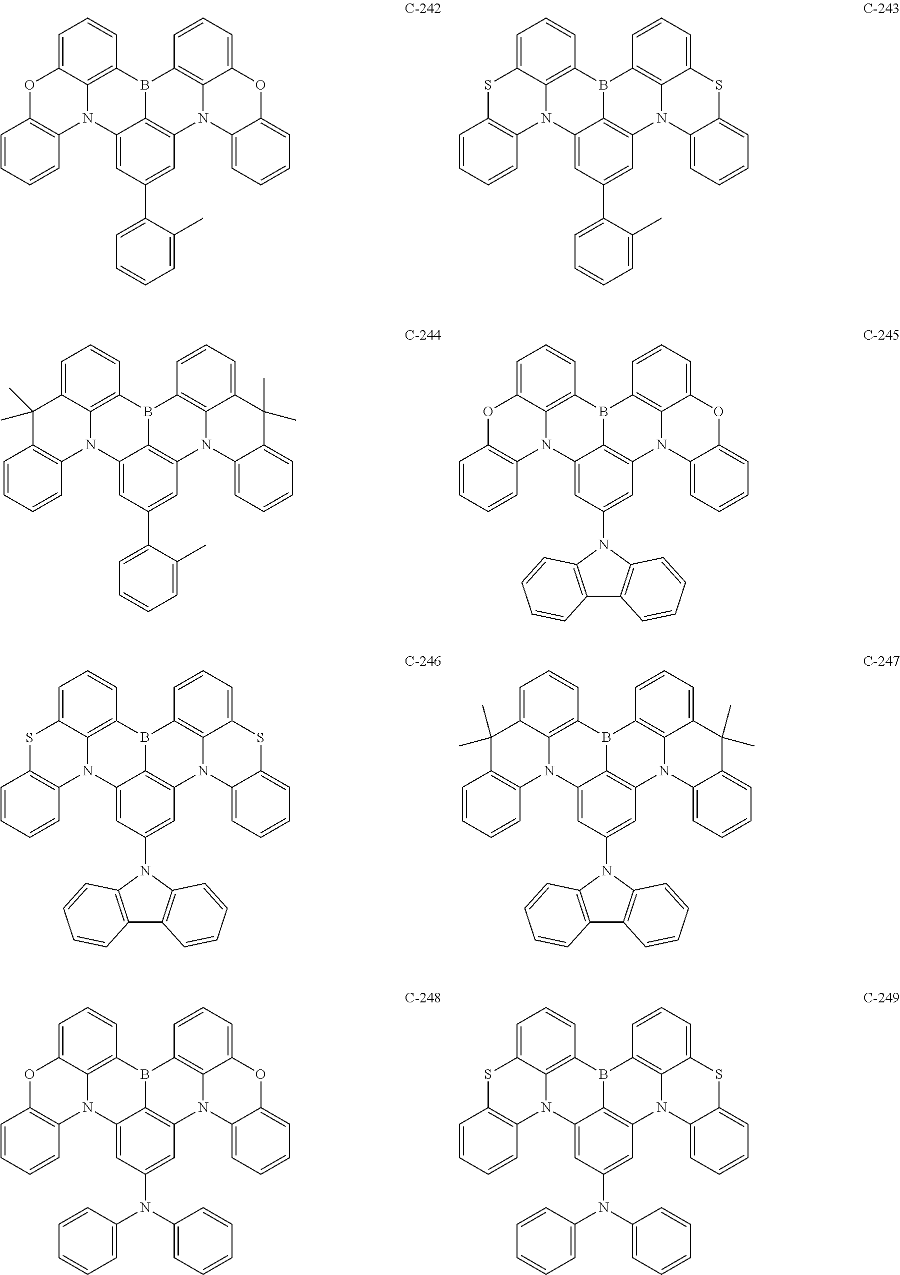

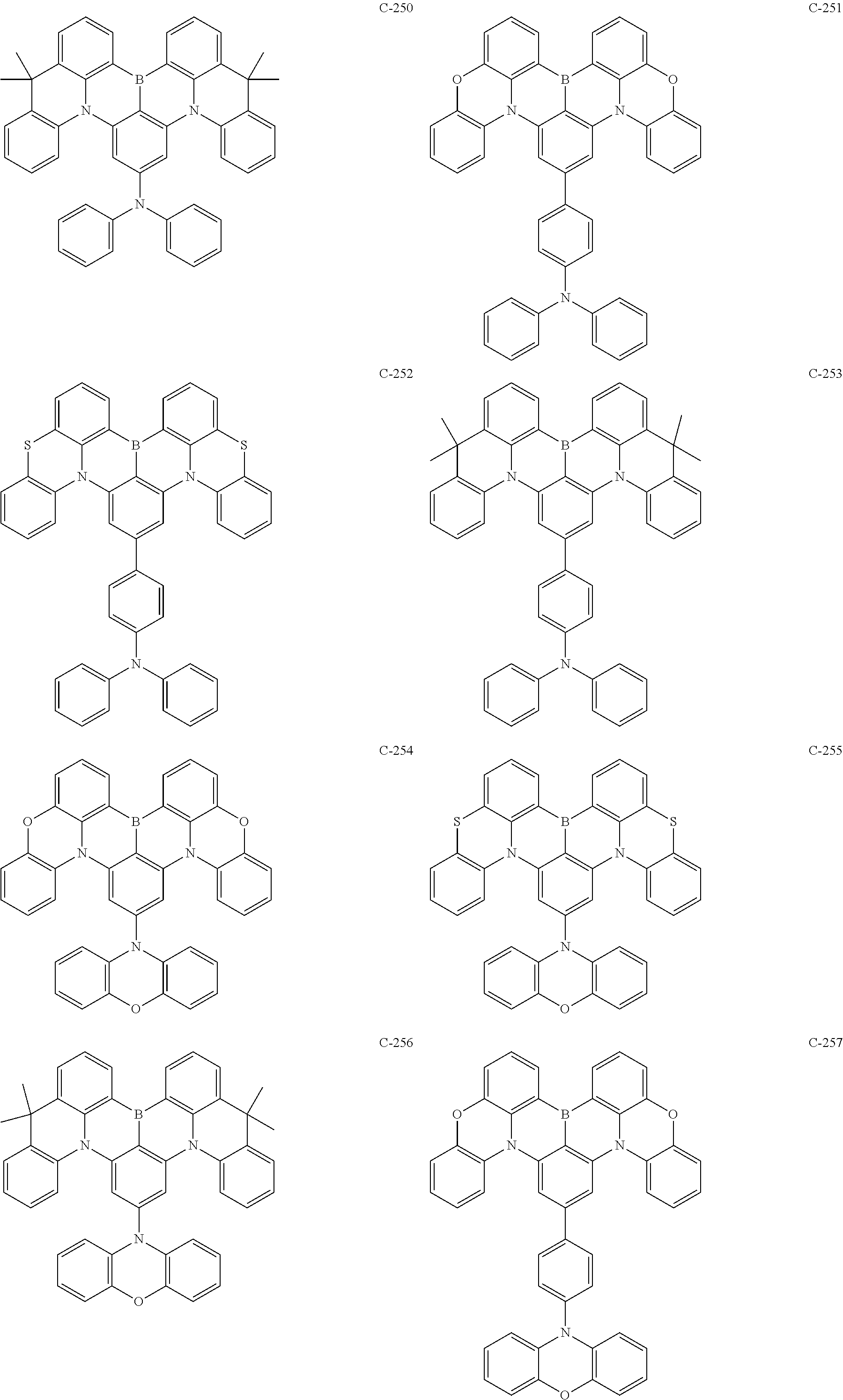

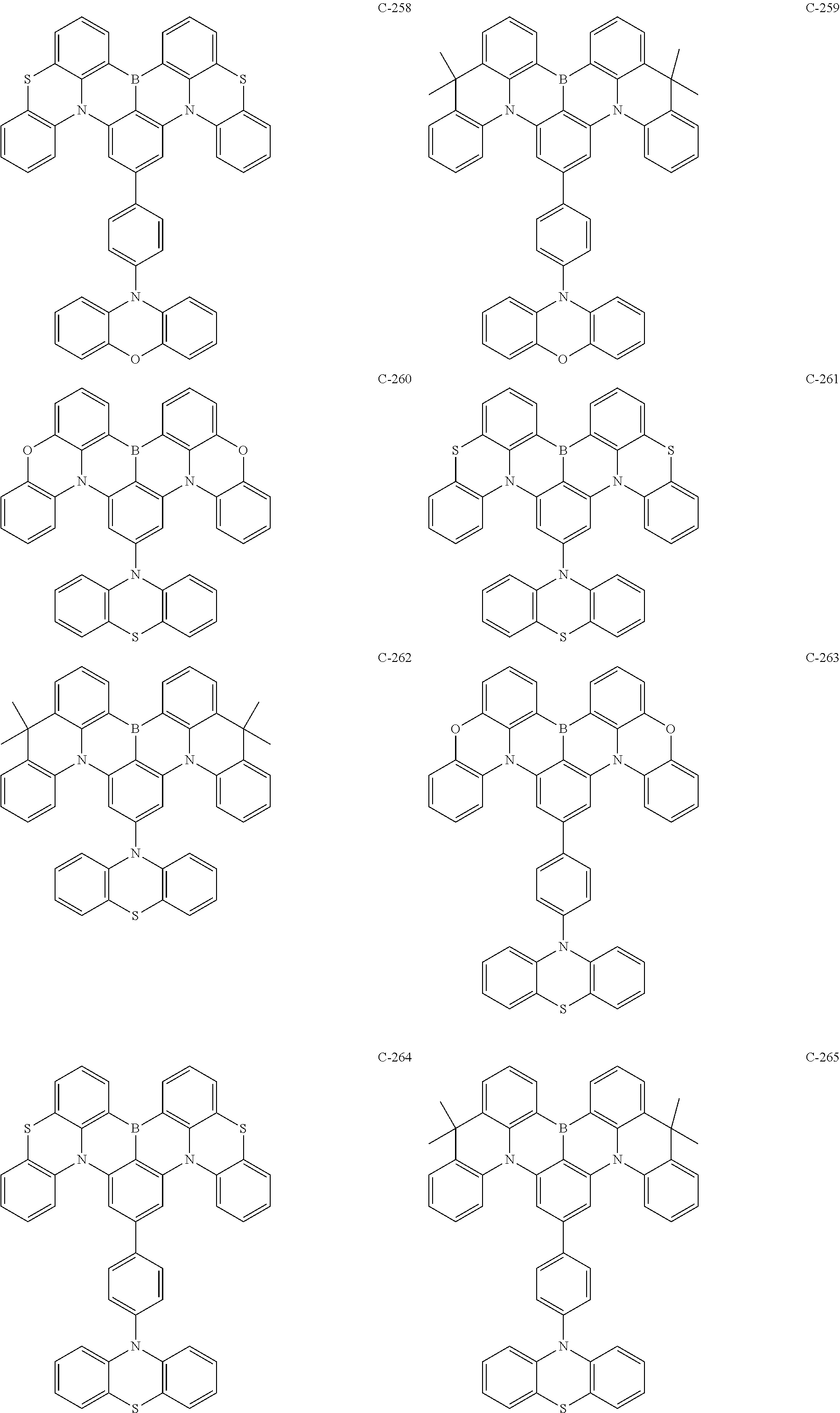

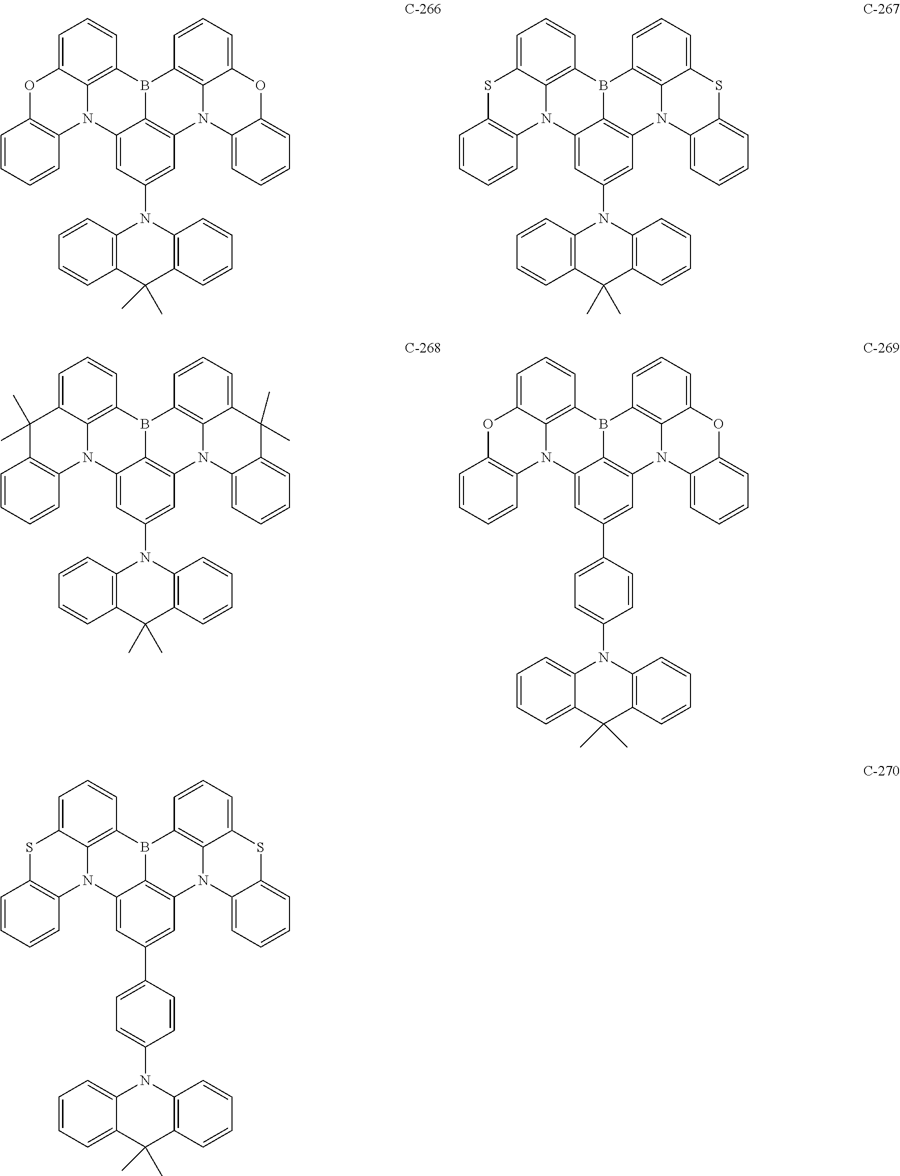 [SMILES: CC1(C)c2ccccc2N(c2cc3c4c(c2)N2c5ccccc5Oc5cccc(c52)B4c2cccc4c2N3c2ccccc2O4)c2ccccc21 ; CC1(C)c2ccccc2N(c2cc3c4c(c2)N2c5ccccc5Sc5cccc(c52)B4c2cccc4c2N3c2ccccc2S4)c2ccccc21 ; CC1(C)c2ccccc2N(c2cc3c4c(c2)N2c5ccccc5C(C)(C)c5cccc(c52)B4c2cccc4c2N3c2ccccc2C4(C)C)c2ccccc21 ; CC1(C)c2ccccc2N(c2ccc(-c3cc4c5c(c3)N3c6ccccc6Oc6cccc(c63)B5c3cccc5c3N4c3ccccc3O5)cc2)c2ccccc21 ; CC1(C)c2ccccc2N(c2ccc(-c3cc4c5c(c3)N3c6ccccc6Sc6cccc(c63)B5c3cccc5c3N4c3ccccc3S5)cc2)c2ccccc21]

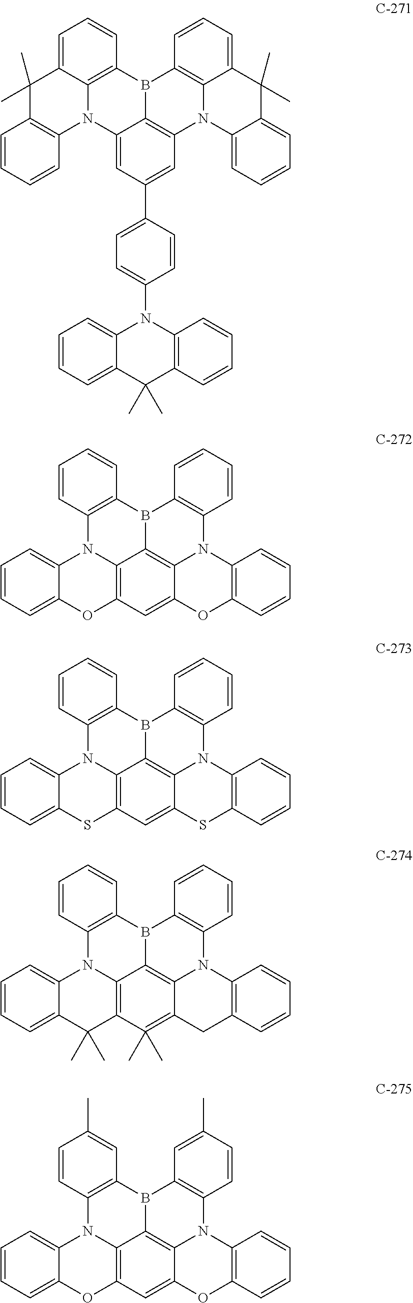

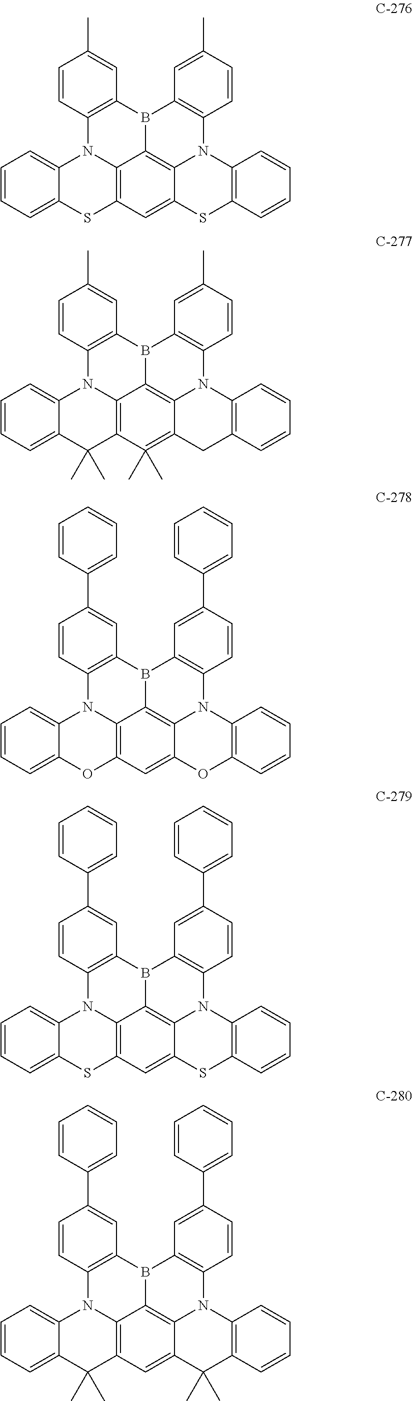

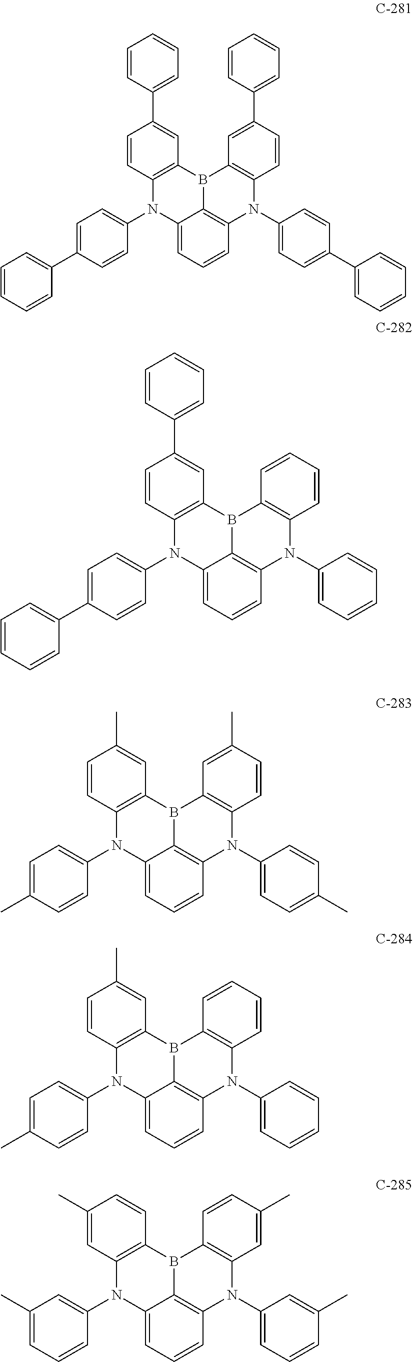

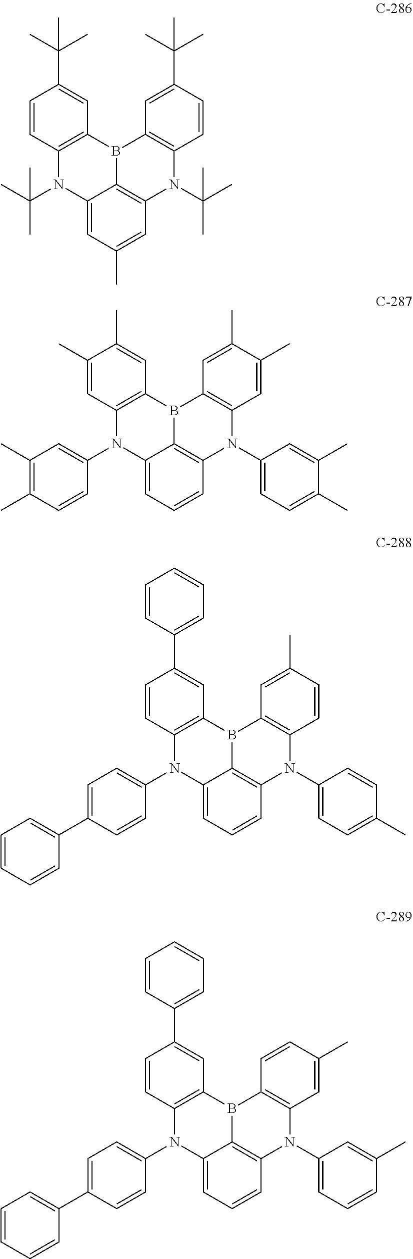

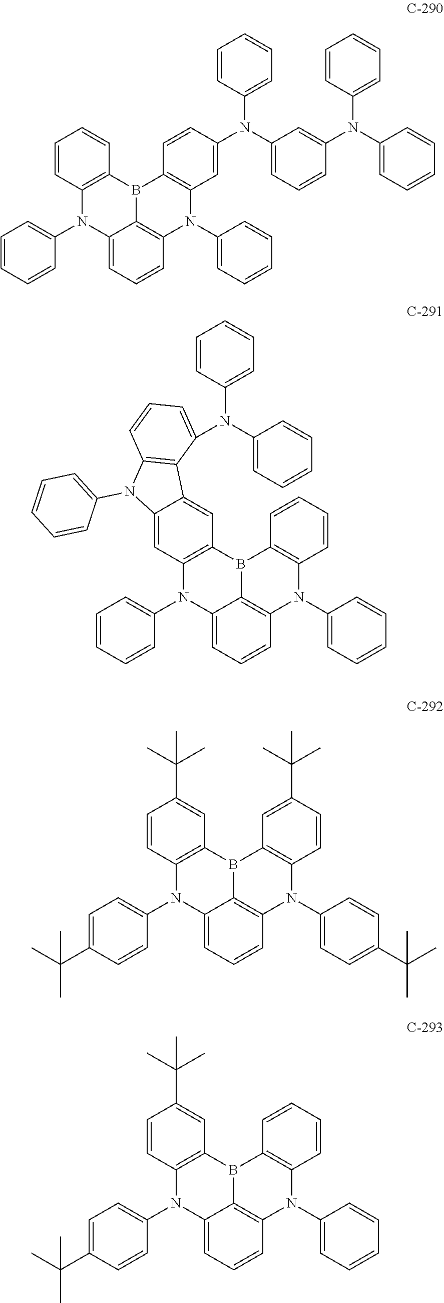

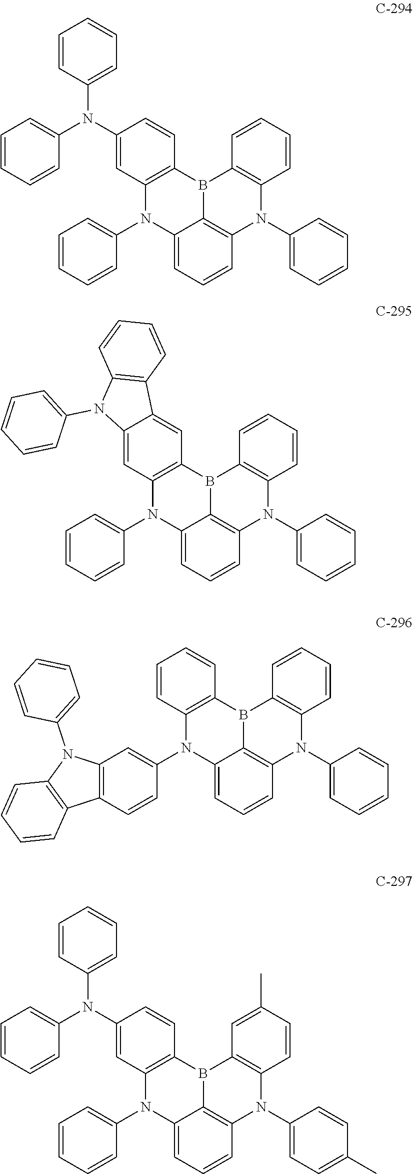 [SMILES: c1ccc(N(c2ccccc2)c2ccc3c(c2)N(c2ccccc2)c2cccc4c2B3c2ccccc2N4c2ccccc2)cc1 ; c1ccc(N2c3ccccc3B3c4cc5c6ccccc6n(-c6ccccc6)c5cc4N(c4ccccc4)c4cccc2c43)cc1 ; c1ccc(N2c3ccccc3B3c4ccccc4N(c4ccc5c6ccccc6n(-c6ccccc6)c5c4)c4cccc2c43)cc1 ; Cc1ccc(N2c3ccc(C)cc3B3c4ccc(N(c5ccccc5)c5ccccc5)cc4N(c4ccccc4)c4cccc2c43)cc1]

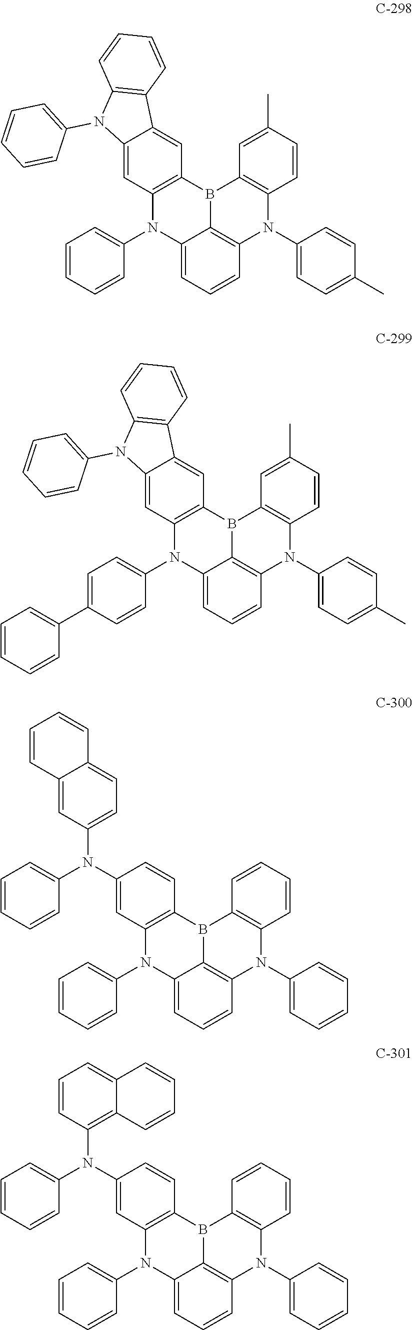

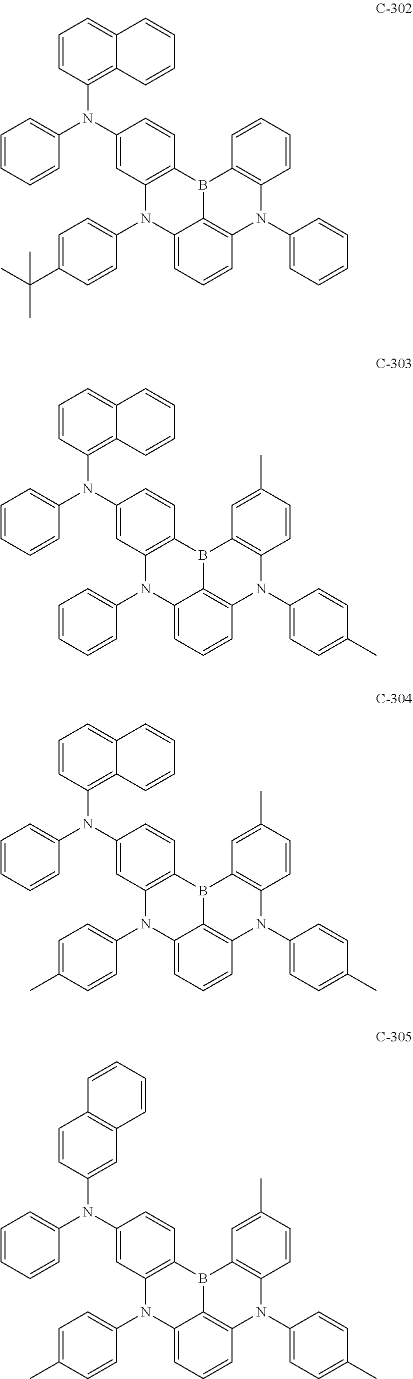 [SMILES: CC(C)(C)c1ccc(N2c3cc(N(c4ccccc4)c4cccc5ccccc45)ccc3B3c4ccccc4N(c4ccccc4)c4cccc2c43)cc1 ; Cc1ccc(N2c3ccc(C)cc3B3c4ccc(N(c5ccccc5)c5cccc6ccccc56)cc4N(c4ccccc4)c4cccc2c43)cc1 ; Cc1ccc(N2c3ccc(C)cc3B3c4ccc(N(c5ccccc5)c5cccc6ccccc56)cc4N(c4ccc(C)cc4)c4cccc2c43)cc1 ; Cc1ccc(N2c3ccc(C)cc3B3c4ccc(N(c5ccccc5)c5ccc6ccccc6c5)cc4N(c4ccc(C)cc4)c4cccc2c43)cc1]

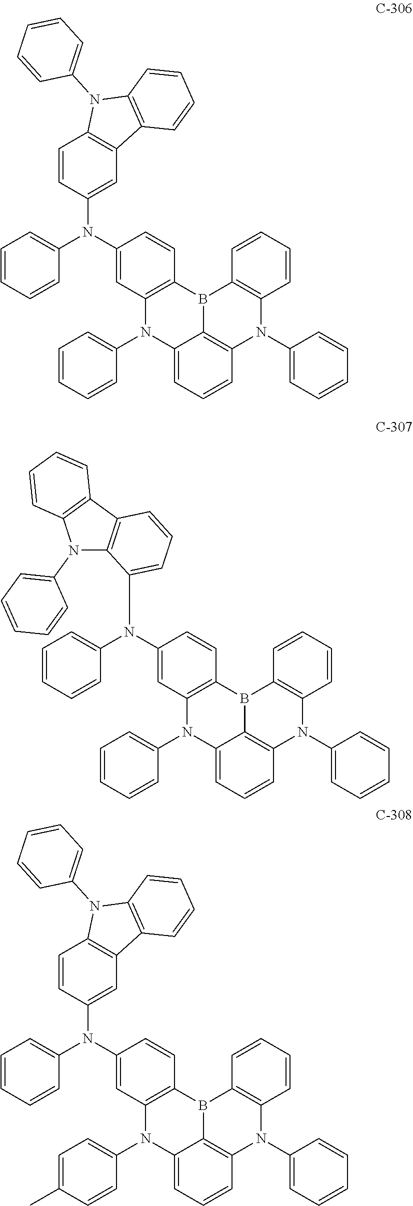 [SMILES: c1ccc(N(c2ccc3c(c2)N(c2ccccc2)c2cccc4c2B3c2ccccc2N4c2ccccc2)c2ccc3c(c2)c2ccccc2n3-c2ccccc2)cc1 ; c1ccc(N2c3ccccc3B3c4ccc(N(c5ccccc5)c5cccc6c7ccccc7n(-c7ccccc7)c56)cc4N(c4ccccc4)c4cccc2c43)cc1 ; Cc1ccc(N2c3cc(N(c4ccccc4)c4ccc5c(c4)c4ccccc4n5-c4ccccc4)ccc3B3c4ccccc4N(c4ccccc4)c4cccc2c43)cc1]

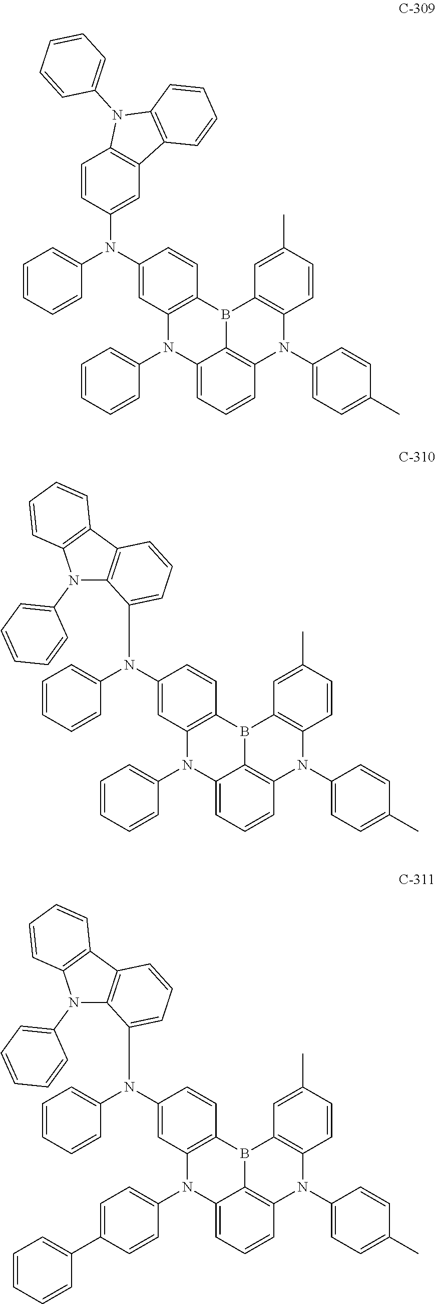 [SMILES: Cc1ccc(N2c3ccc(C)cc3B3c4ccc(N(c5ccccc5)c5ccc6c(c5)c5ccccc5n6-c5ccccc5)cc4N(c4ccccc4)c4cccc2c43)cc1 ; Cc1ccc(N2c3ccc(C)cc3B3c4ccc(N(c5ccccc5)c5cccc6c7ccccc7n(-c7ccccc7)c56)cc4N(c4ccccc4)c4cccc2c43)cc1 ; Cc1ccc(N2c3ccc(C)cc3B3c4ccc(N(c5ccccc5)c5cccc6c7ccccc7n(-c7ccccc7)c56)cc4N(c4ccc(-c5ccccc5)cc4)c4cccc2c43)cc1]

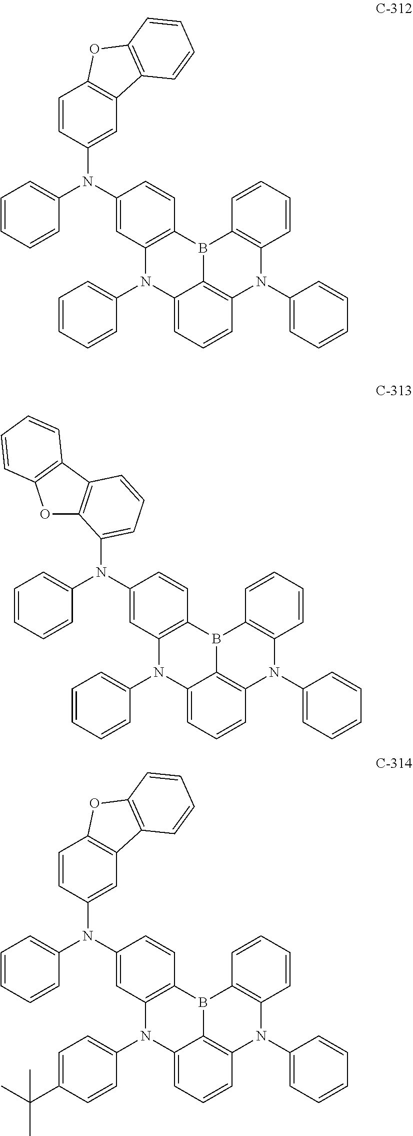 [SMILES: c1ccc(N(c2ccc3c(c2)N(c2ccccc2)c2cccc4c2B3c2ccccc2N4c2ccccc2)c2ccc3oc4ccccc4c3c2)cc1 ; c1ccc(N2c3ccccc3B3c4ccc(N(c5ccccc5)c5cccc6c5oc5ccccc56)cc4N(c4ccccc4)c4cccc2c43)cc1 ; CC(C)(C)c1ccc(N2c3cc(N(c4ccccc4)c4ccc5oc6ccccc6c5c4)ccc3B3c4ccccc4N(c4ccccc4)c4cccc2c43)cc1]

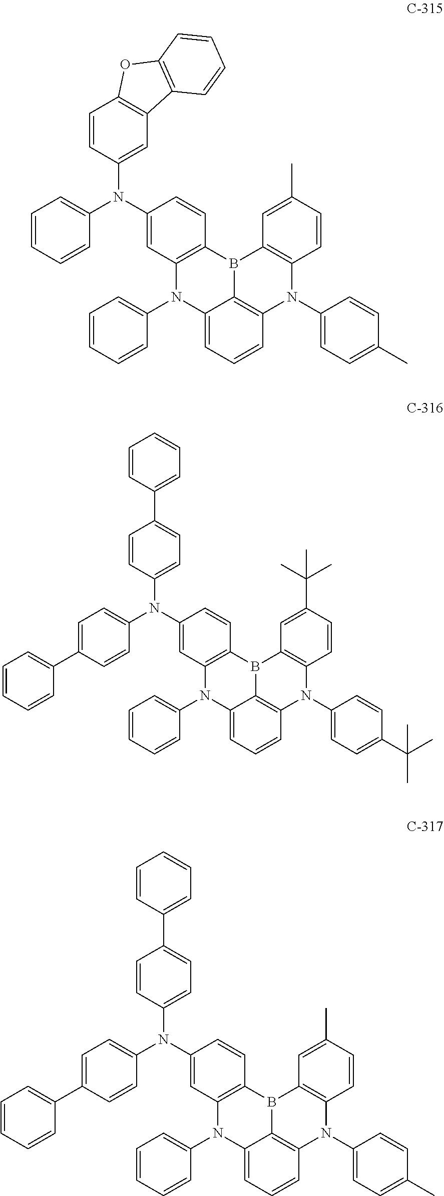 [SMILES: Cc1ccc(N2c3ccc(C)cc3B3c4ccc(N(c5ccccc5)c5ccc6oc7ccccc7c6c5)cc4N(c4ccccc4)c4cccc2c43)cc1 ; CC(C)(C)c1ccc(N2c3ccc(C(C)(C)C)cc3B3c4ccc(N(c5ccc(-c6ccccc6)cc5)c5ccc(-c6ccccc6)cc5)cc4N(c4ccccc4)c4cccc2c43)cc1 ; Cc1ccc(N2c3ccc(C)cc3B3c4ccc(N(c5ccc(-c6ccccc6)cc5)c5ccc(-c6ccccc6)cc5)cc4N(c4ccccc4)c4cccc2c43)cc1]

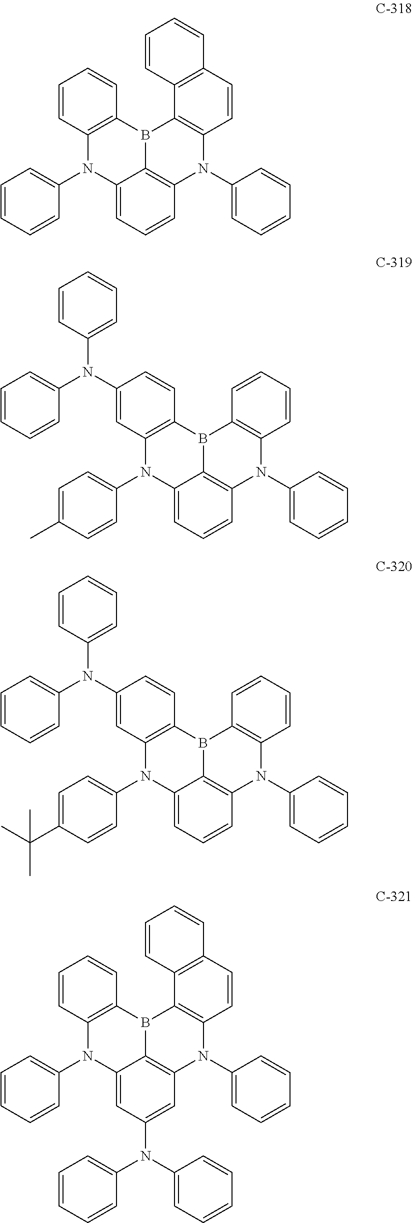

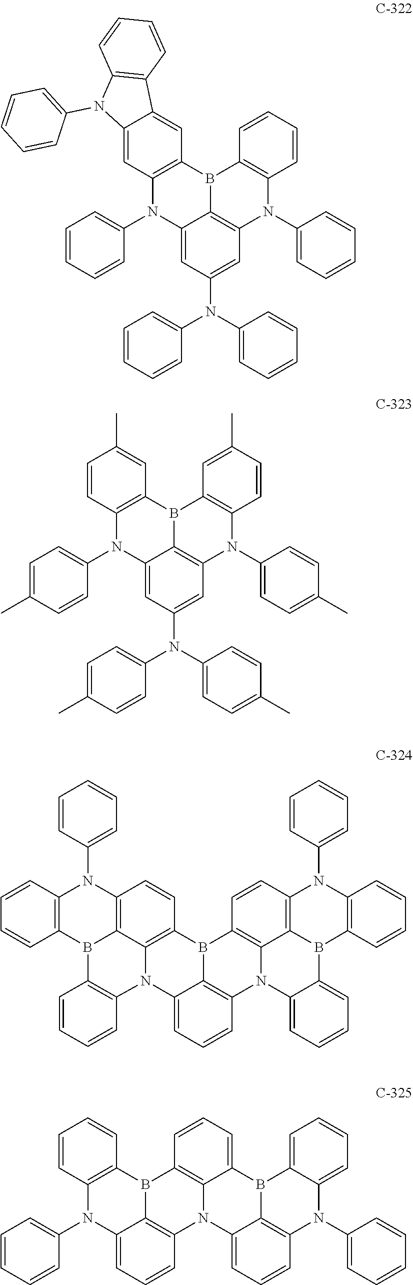

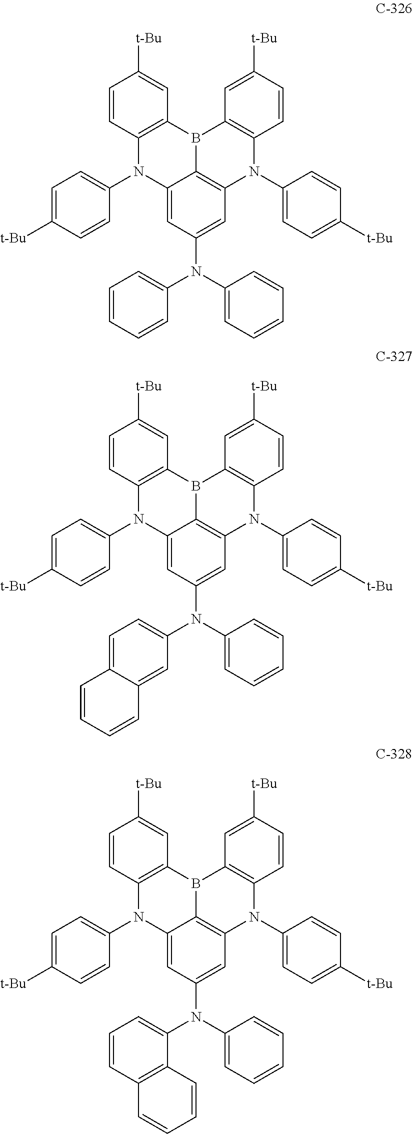 [SMILES: CC(C)(C)c1ccc(N2c3ccc(C(C)(C)C)cc3B3c4cc(C(C)(C)C)ccc4N(c4ccc(C(C)(C)C)cc4)c4cc(N(c5ccccc5)c5ccccc5)cc2c43)cc1 ; CC(C)(C)c1ccc(N2c3ccc(C(C)(C)C)cc3B3c4cc(C(C)(C)C)ccc4N(c4ccc(C(C)(C)C)cc4)c4cc(N(c5ccccc5)c5ccc6ccccc6c5)cc2c43)cc1 ; CC(C)(C)c1ccc(N2c3ccc(C(C)(C)C)cc3B3c4cc(C(C)(C)C)ccc4N(c4ccc(C(C)(C)C)cc4)c4cc(N(c5ccccc5)c5cccc6ccccc56)cc2c43)cc1]

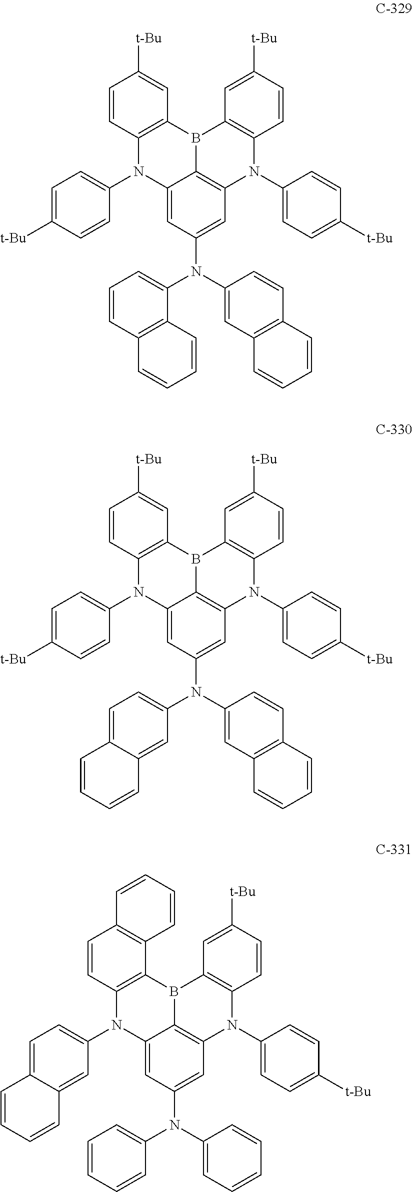 [SMILES: CC(C)(C)c1ccc(N2c3ccc(C(C)(C)C)cc3B3c4cc(C(C)(C)C)ccc4N(c4ccc(C(C)(C)C)cc4)c4cc(N(c5ccc6ccccc6c5)c5cccc6ccccc56)cc2c43)cc1 ; CC(C)(C)c1ccc(N2c3ccc(C(C)(C)C)cc3B3c4cc(C(C)(C)C)ccc4N(c4ccc(C(C)(C)C)cc4)c4cc(N(c5ccc6ccccc6c5)c5ccc6ccccc6c5)cc2c43)cc1 ; CC(C)(C)c1ccc(N2c3ccc(C(C)(C)C)cc3B3c4c2cc(N(c2ccccc2)c2ccccc2)cc4N(c2ccc4ccccc4c2)c2ccc4ccccc4c23)cc1]

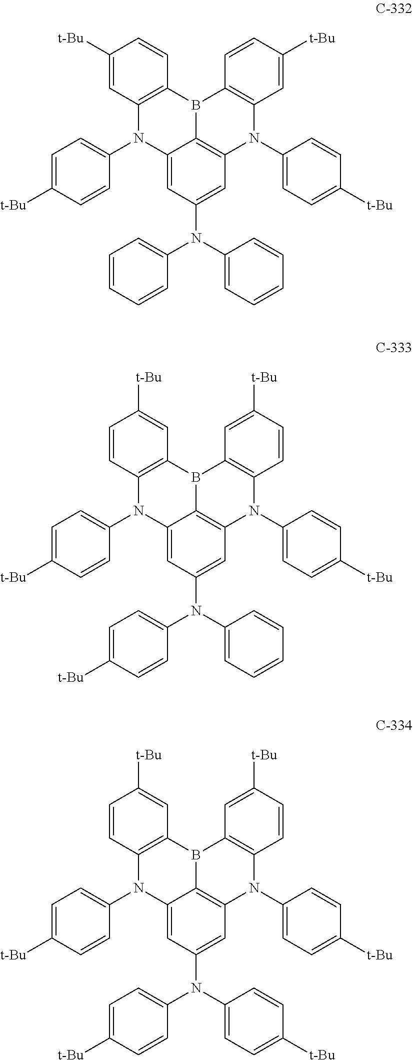 [SMILES: CC(C)(C)c1ccc(N2c3cc(C(C)(C)C)ccc3B3c4ccc(C(C)(C)C)cc4N(c4ccc(C(C)(C)C)cc4)c4cc(N(c5ccccc5)c5ccccc5)cc2c43)cc1 ; CC(C)(C)c1ccc(N(c2ccccc2)c2cc3c4c(c2)N(c2ccc(C(C)(C)C)cc2)c2ccc(C(C)(C)C)cc2B4c2cc(C(C)(C)C)ccc2N3c2ccc(C(C)(C)C)cc2)cc1 ; CC(C)(C)c1ccc(N(c2ccc(C(C)(C)C)cc2)c2cc3c4c(c2)N(c2ccc(C(C)(C)C)cc2)c2ccc(C(C)(C)C)cc2B4c2cc(C(C)(C)C)ccc2N3c2ccc(C(C)(C)C)cc2)cc1]

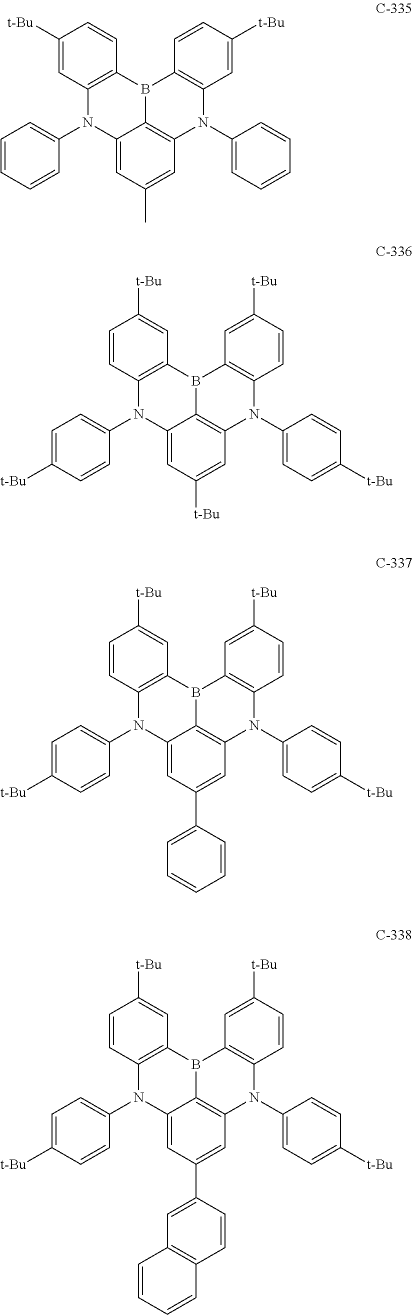 [SMILES: Cc1cc2c3c(c1)N(c1ccccc1)c1cc(C(C)(C)C)ccc1B3c1ccc(C(C)(C)C)cc1N2c1ccccc1 ; CC(C)(C)c1ccc(N2c3ccc(C(C)(C)C)cc3B3c4cc(C(C)(C)C)ccc4N(c4ccc(C(C)(C)C)cc4)c4cc(C(C)(C)C)cc2c43)cc1 ; CC(C)(C)c1ccc(N2c3ccc(C(C)(C)C)cc3B3c4cc(C(C)(C)C)ccc4N(c4ccc(C(C)(C)C)cc4)c4cc(-c5ccccc5)cc2c43)cc1 ; CC(C)(C)c1ccc(N2c3ccc(C(C)(C)C)cc3B3c4cc(C(C)(C)C)ccc4N(c4ccc(C(C)(C)C)cc4)c4cc(-c5ccc6ccccc6c5)cc2c43)cc1]

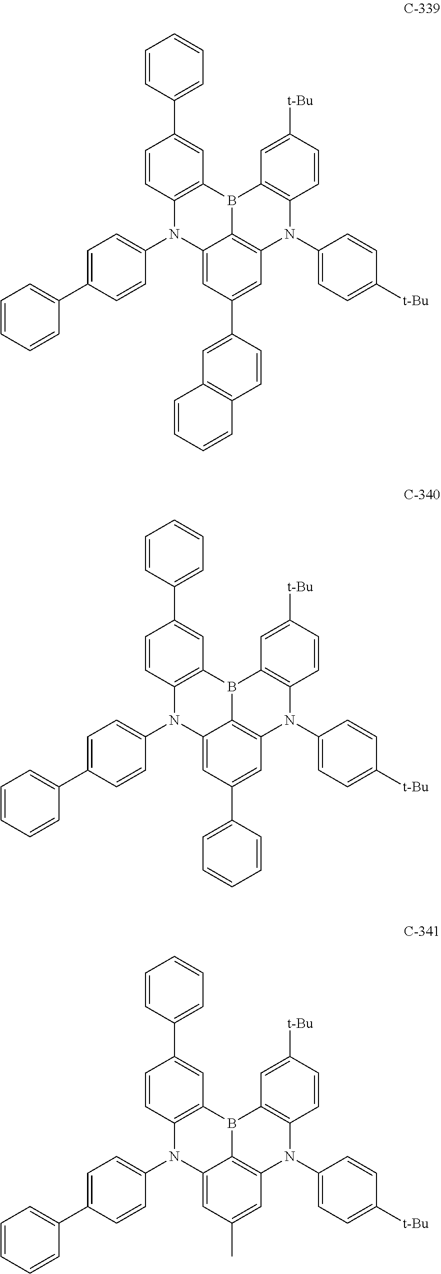 [SMILES: CC(C)(C)c1ccc(N2c3ccc(C(C)(C)C)cc3B3c4cc(-c5ccccc5)ccc4N(c4ccc(-c5ccccc5)cc4)c4cc(-c5ccc6ccccc6c5)cc2c43)cc1 ; CC(C)(C)c1ccc(N2c3ccc(C(C)(C)C)cc3B3c4cc(-c5ccccc5)ccc4N(c4ccc(-c5ccccc5)cc4)c4cc(-c5ccccc5)cc2c43)cc1 ; Cc1cc2c3c(c1)N(c1ccc(C(C)(C)C)cc1)c1ccc(C(C)(C)C)cc1B3c1cc(-c3ccccc3)ccc1N2c1ccc(-c2ccccc2)cc1]

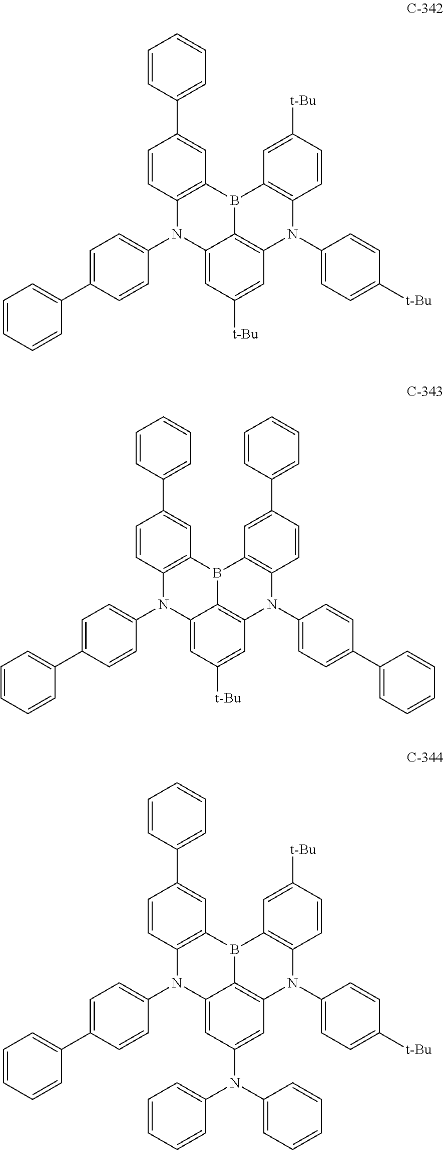 [SMILES: CC(C)(C)c1ccc(N2c3ccc(C(C)(C)C)cc3B3c4cc(-c5ccccc5)ccc4N(c4ccc(-c5ccccc5)cc4)c4cc(C(C)(C)C)cc2c43)cc1 ; CC(C)(C)c1cc2c3c(c1)N(c1ccc(-c4ccccc4)cc1)c1ccc(-c4ccccc4)cc1B3c1cc(-c3ccccc3)ccc1N2c1ccc(-c2ccccc2)cc1 ; CC(C)(C)c1ccc(N2c3ccc(C(C)(C)C)cc3B3c4cc(-c5ccccc5)ccc4N(c4ccc(-c5ccccc5)cc4)c4cc(N(c5ccccc5)c5ccccc5)cc2c43)cc1]

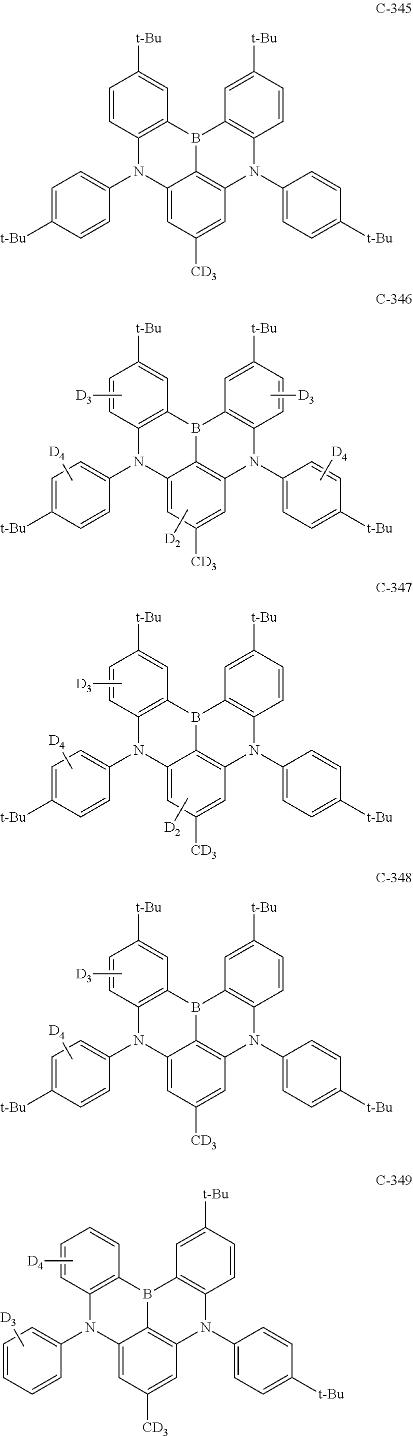

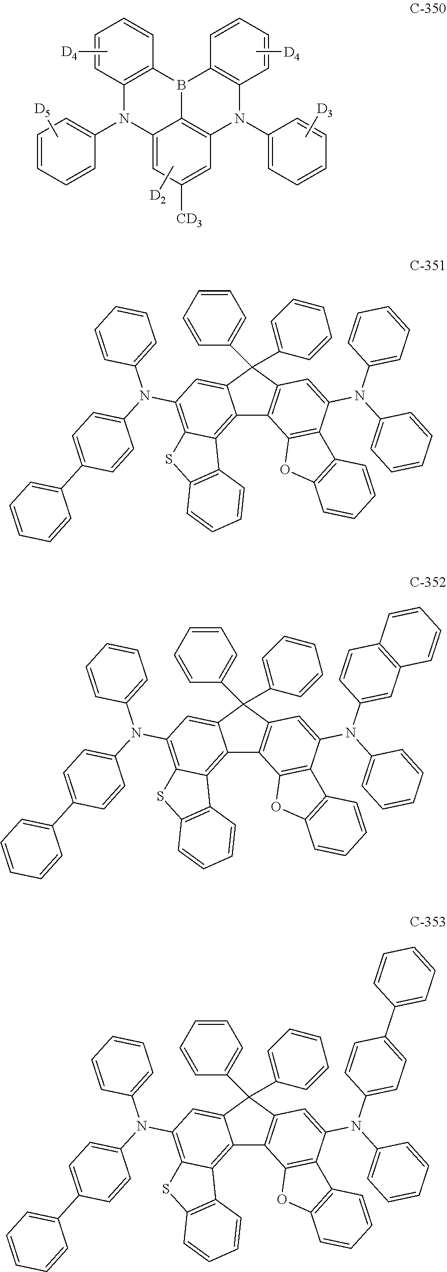

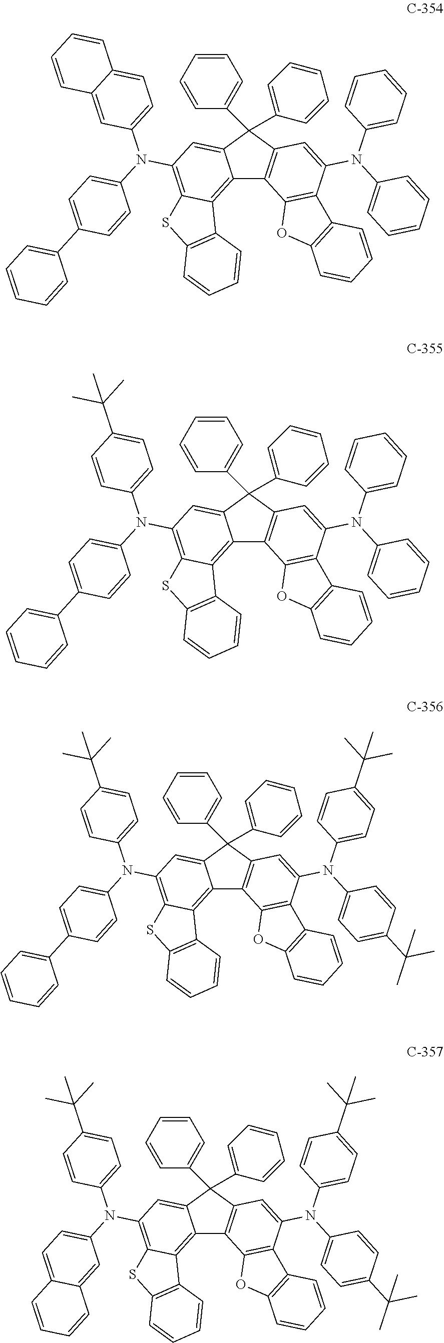

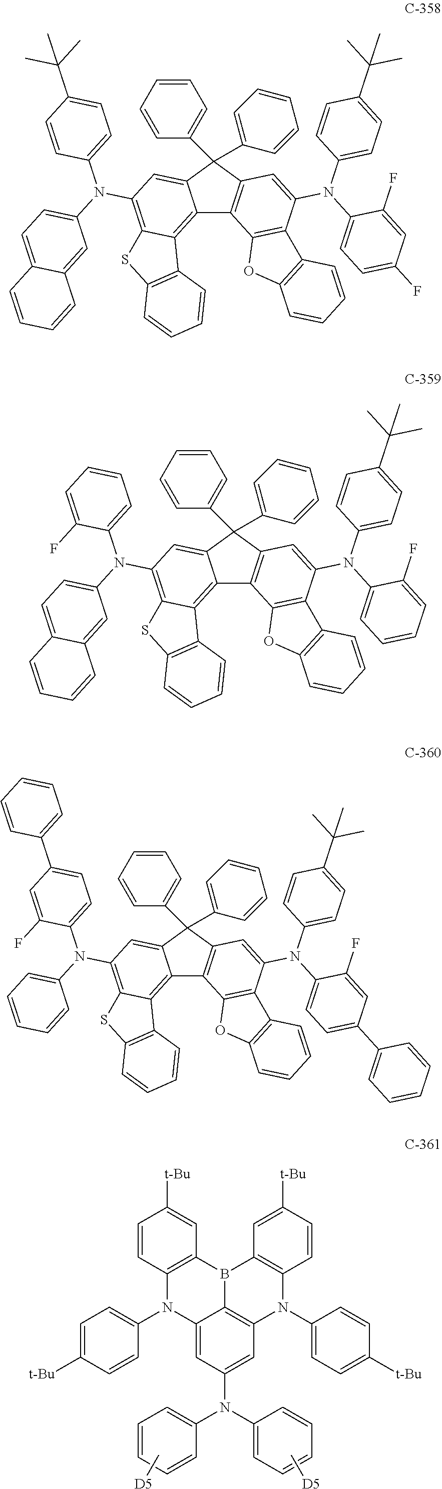

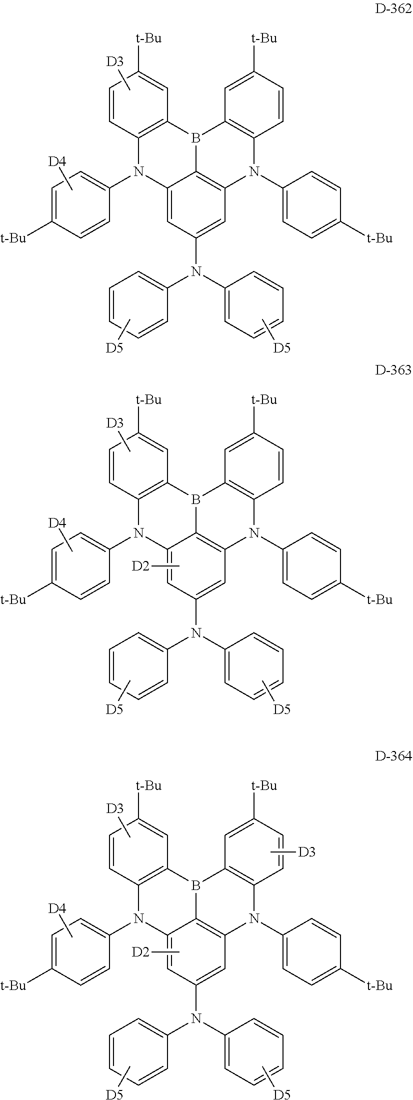

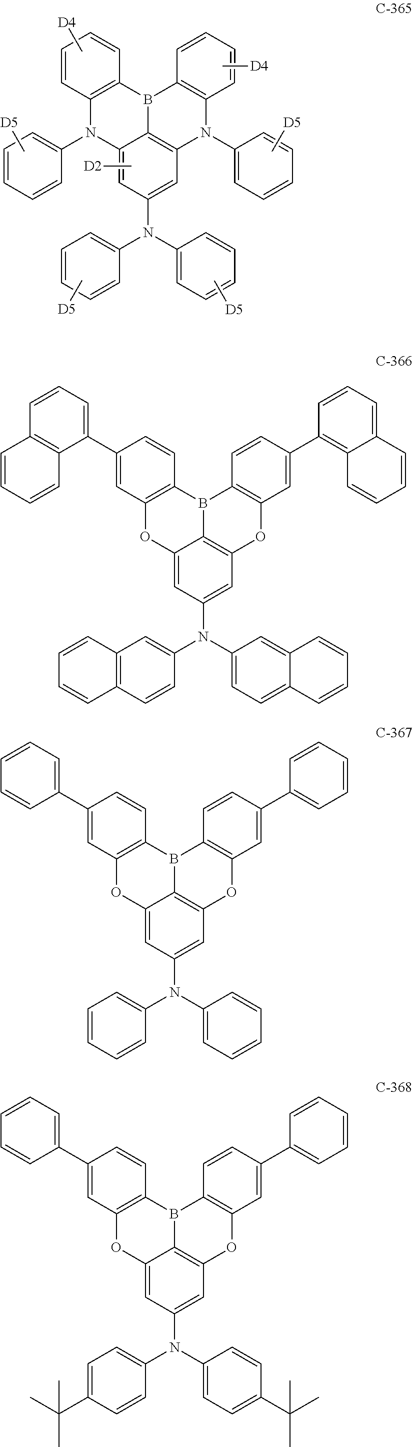

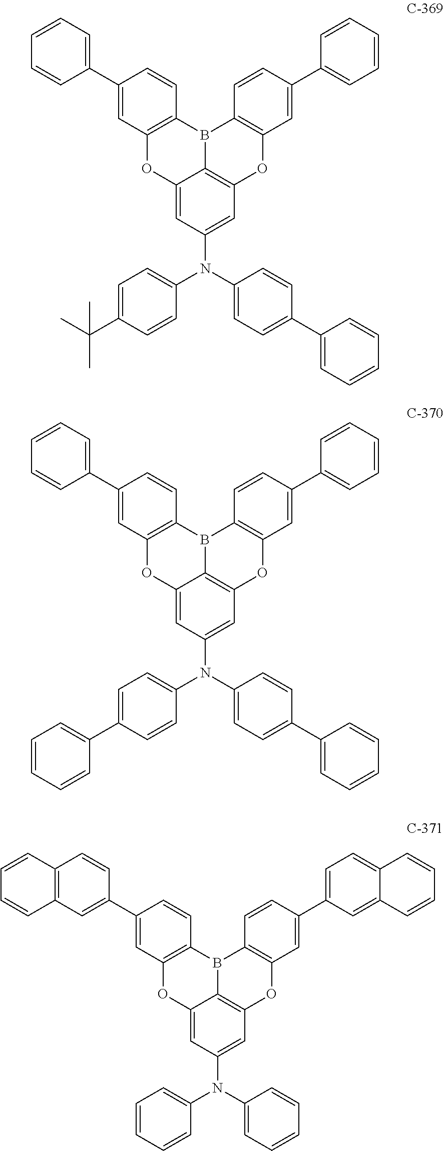

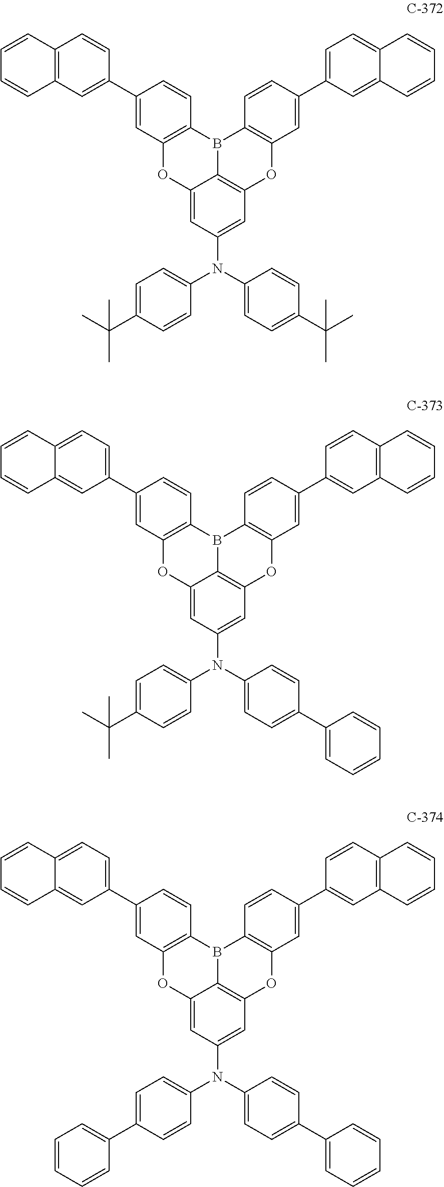 [SMILES: CC(C)(C)c1ccc(N(c2ccc(C(C)(C)C)cc2)c2cc3c4c(c2)Oc2cc(-c5ccc6ccccc6c5)ccc2B4c2ccc(-c4ccc5ccccc5c4)cc2O3)cc1 ; CC(C)(C)c1ccc(N(c2ccc(-c3ccccc3)cc2)c2cc3c4c(c2)Oc2cc(-c5ccc6ccccc6c5)ccc2B4c2ccc(-c4ccc5ccccc5c4)cc2O3)cc1 ; c1ccc(-c2ccc(N(c3ccc(-c4ccccc4)cc3)c3cc4c5c(c3)Oc3cc(-c6ccc7ccccc7c6)ccc3B5c3ccc(-c5ccc6ccccc6c5)cc3O4)cc2)cc1]

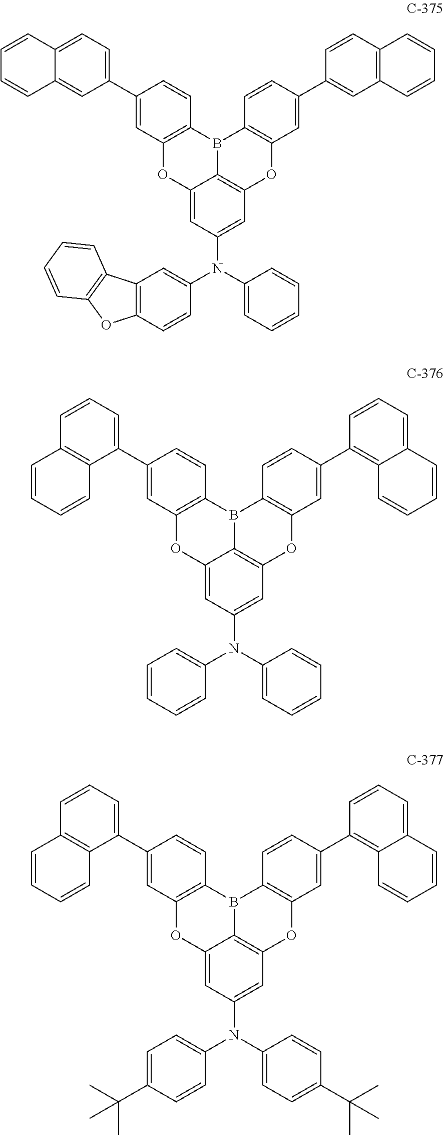 [SMILES: c1ccc(N(c2cc3c4c(c2)Oc2cc(-c5ccc6ccccc6c5)ccc2B4c2ccc(-c4ccc5ccccc5c4)cc2O3)c2ccc3oc4ccccc4c3c2)cc1 ; c1ccc(N(c2ccccc2)c2cc3c4c(c2)Oc2cc(-c5cccc6ccccc56)ccc2B4c2ccc(-c4cccc5ccccc45)cc2O3)cc1 ; CC(C)(C)c1ccc(N(c2ccc(C(C)(C)C)cc2)c2cc3c4c(c2)Oc2cc(-c5cccc6ccccc56)ccc2B4c2ccc(-c4cccc5ccccc45)cc2O3)cc1]

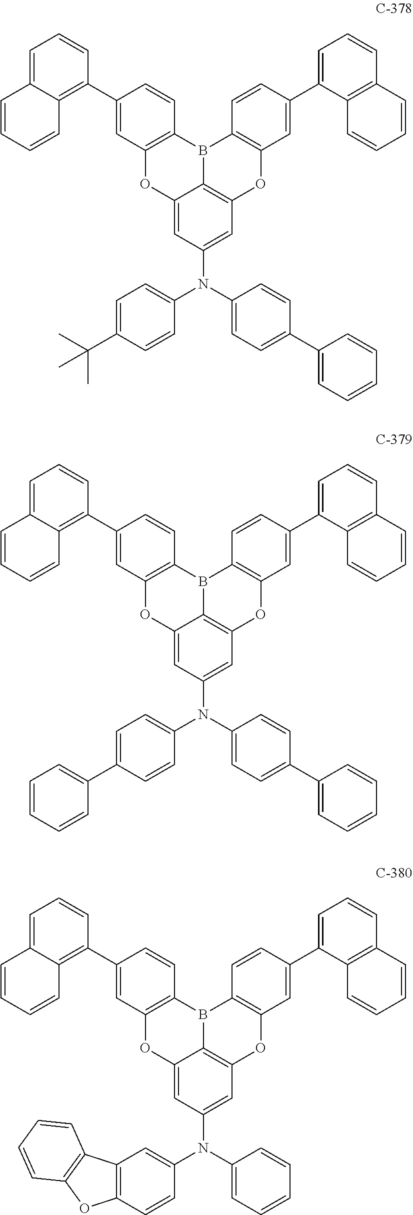 [SMILES: CC(C)(C)c1ccc(N(c2ccc(-c3ccccc3)cc2)c2cc3c4c(c2)Oc2cc(-c5cccc6ccccc56)ccc2B4c2ccc(-c4cccc5ccccc45)cc2O3)cc1 ; c1ccc(-c2ccc(N(c3ccc(-c4ccccc4)cc3)c3cc4c5c(c3)Oc3cc(-c6cccc7ccccc67)ccc3B5c3ccc(-c5cccc6ccccc56)cc3O4)cc2)cc1 ; c1ccc(N(c2cc3c4c(c2)Oc2cc(-c5cccc6ccccc56)ccc2B4c2ccc(-c4cccc5ccccc45)cc2O3)c2ccc3oc4ccccc4c3c2)cc1]

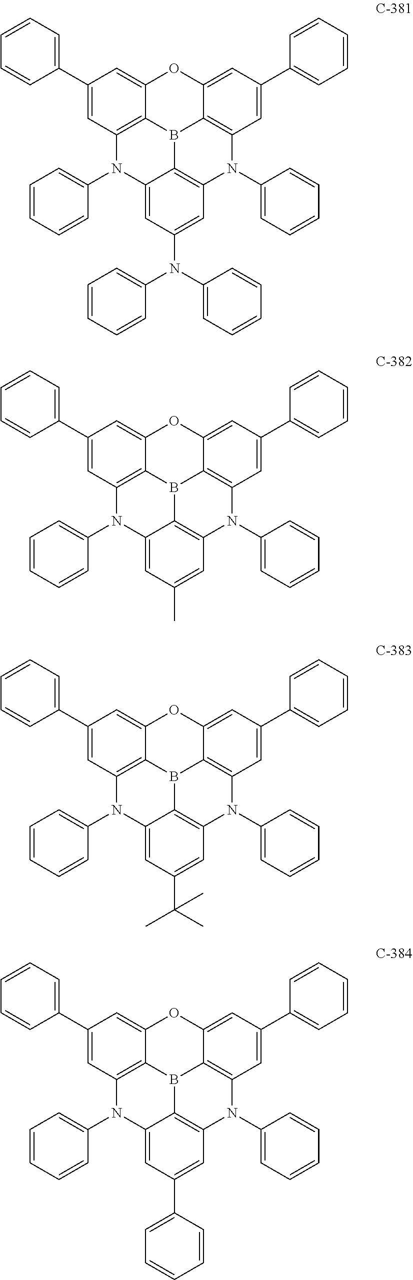 [SMILES: c1ccc(-c2cc3c4c(c2)N(c2ccccc2)c2cc(N(c5ccccc5)c5ccccc5)cc5c2B4c2c(cc(-c4ccccc4)cc2N5c2ccccc2)O3)cc1 ; Cc1cc2c3c(c1)N(c1ccccc1)c1cc(-c4ccccc4)cc4c1B3c1c(cc(-c3ccccc3)cc1N2c1ccccc1)O4 ; CC(C)(C)c1cc2c3c(c1)N(c1ccccc1)c1cc(-c4ccccc4)cc4c1B3c1c(cc(-c3ccccc3)cc1N2c1ccccc1)O4 ; c1ccc(-c2cc3c4c(c2)N(c2ccccc2)c2cc(-c5ccccc5)cc5c2B4c2c(cc(-c4ccccc4)cc2N5c2ccccc2)O3)cc1]

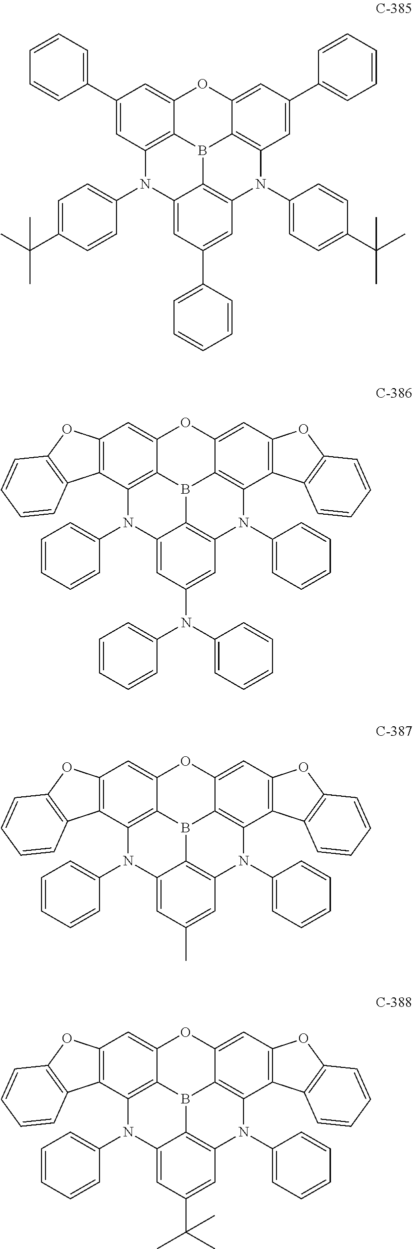 [SMILES: CC(C)(C)c1ccc(N2c3cc(-c4ccccc4)cc4c3B3c5c(cc(-c6ccccc6)cc5N(c5ccc(C(C)(C)C)cc5)c5cc(-c6ccccc6)cc2c53)O4)cc1 ; c1ccc(N(c2ccccc2)c2cc3c4c(c2)N(c2ccccc2)c2c5c(cc6oc7ccccc7c26)Oc2cc6oc7ccccc7c6c(c2B45)N3c2ccccc2)cc1 ; Cc1cc2c3c(c1)N(c1ccccc1)c1c4c(cc5oc6ccccc6c15)Oc1cc5oc6ccccc6c5c(c1B34)N2c1ccccc1 ; CC(C)(C)c1cc2c3c(c1)N(c1ccccc1)c1c4c(cc5oc6ccccc6c15)Oc1cc5oc6ccccc6c5c(c1B34)N2c1ccccc1]

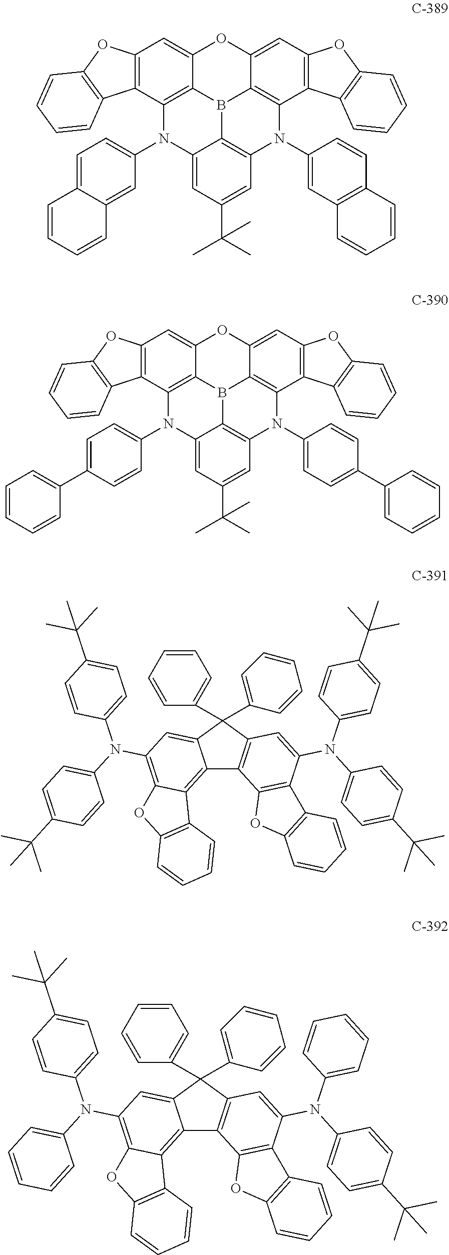 [SMILES: CC(C)(C)c1cc2c3c(c1)N(c1ccc4ccccc4c1)c1c4c(cc5oc6ccccc6c15)Oc1cc5oc6ccccc6c5c(c1B34)N2c1ccc2ccccc2c1 ; CC(C)(C)c1cc2c3c(c1)N(c1ccc(-c4ccccc4)cc1)c1c4c(cc5oc6ccccc6c15)Oc1cc5oc6ccccc6c5c(c1B34)N2c1ccc(-c2ccccc2)cc1 ; CC(C)(C)c1ccc(N(c2ccc(C(C)(C)C)cc2)c2cc3c(c4c2oc2ccccc24)-c2c(cc(N(c4ccc(C(C)(C)C)cc4)c4ccc(C(C)(C)C)cc4)c4c2oc2ccccc24)C3(c2ccccc2)c2ccccc2)cc1 ; CC(C)(C)c1ccc(N(c2ccccc2)c2cc3c(c4c2oc2ccccc24)-c2c(cc(N(c4ccccc4)c4ccc(C(C)(C)C)cc4)c4c2oc2ccccc24)C3(c2ccccc2)c2ccccc2)cc1]

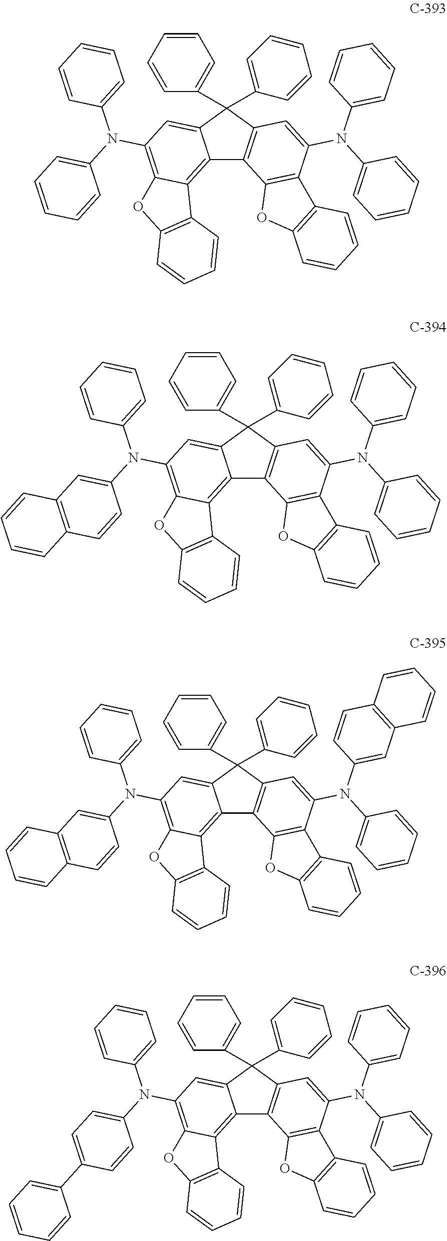 [SMILES: c1ccc(N(c2ccccc2)c2cc3c(c4c2oc2ccccc24)-c2c(cc(N(c4ccccc4)c4ccccc4)c4c2oc2ccccc24)C3(c2ccccc2)c2ccccc2)cc1 ; c1ccc(N(c2ccc3ccccc3c2)c2cc3c(c4c2oc2ccccc24)-c2c(cc(N(c4ccccc4)c4ccccc4)c4c2oc2ccccc24)C3(c2ccccc2)c2ccccc2)cc1 ; c1ccc(N(c2ccc3ccccc3c2)c2cc3c(c4c2oc2ccccc24)-c2c(cc(N(c4ccccc4)c4ccc5ccccc5c4)c4c2oc2ccccc24)C3(c2ccccc2)c2ccccc2)cc1 ; c1ccc(-c2ccc(N(c3ccccc3)c3cc4c(c5c3oc3ccccc35)-c3c(cc(N(c5ccccc5)c5ccccc5)c5c3oc3ccccc35)C4(c3ccccc3)c3ccccc3)cc2)cc1]

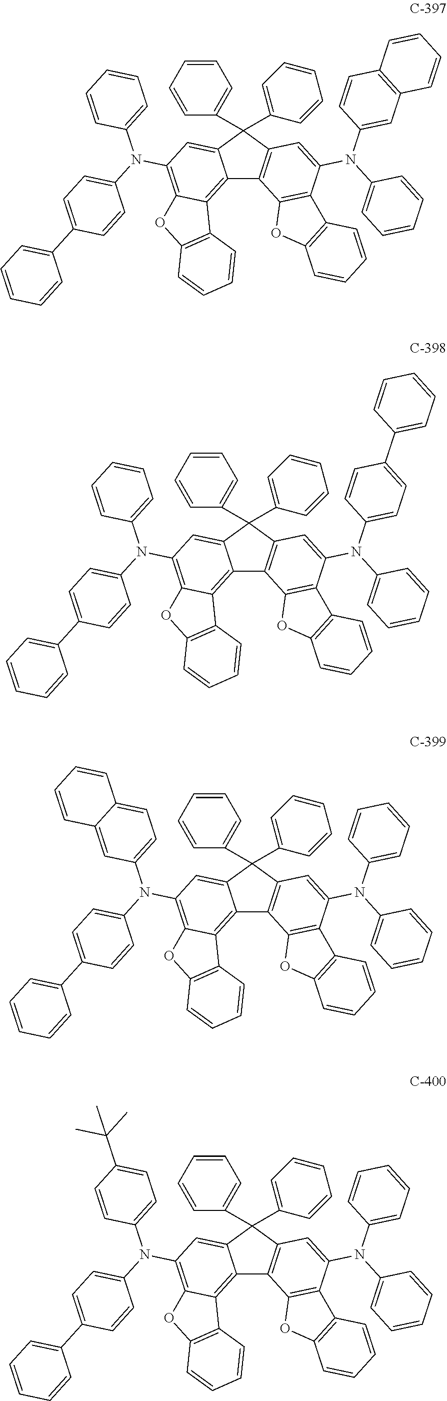

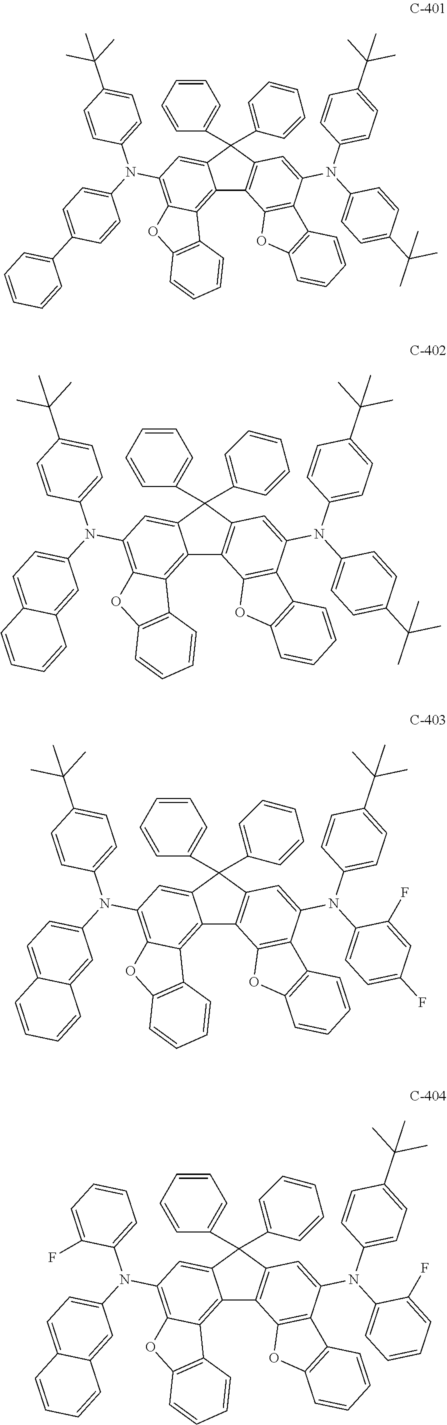

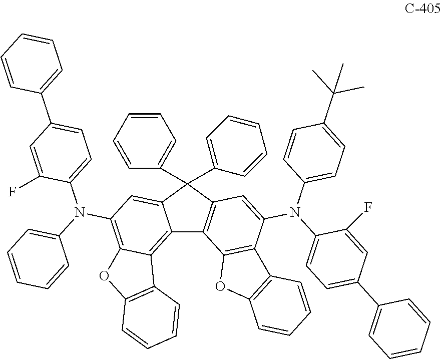 [SMILES: CC(C)(C)c1ccc(N(c2ccc(-c3ccccc3)cc2F)c2cc3c(c4oc5ccccc5c24)-c2c(cc(N(c4ccccc4)c4ccc(-c5ccccc5)cc4F)c4oc5ccccc5c24)C3(c2ccccc2)c2ccccc2)cc1]

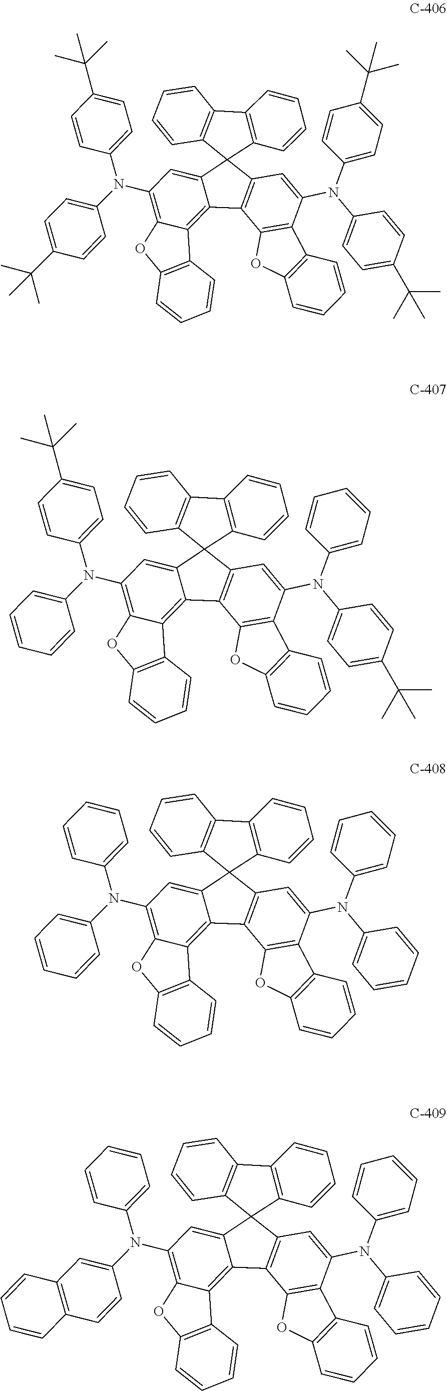 [SMILES: CC(C)(C)c1ccc(N(c2ccc(C(C)(C)C)cc2)c2cc3c(c4c2oc2ccccc24)-c2c(cc(N(c4ccc(C(C)(C)C)cc4)c4ccc(C(C)(C)C)cc4)c4c2oc2ccccc24)C32c3ccccc3-c3ccccc32)cc1 ; CC(C)(C)c1ccc(N(c2ccccc2)c2cc3c(c4c2oc2ccccc24)-c2c(cc(N(c4ccccc4)c4ccc(C(C)(C)C)cc4)c4c2oc2ccccc24)C32c3ccccc3-c3ccccc32)cc1 ; c1ccc(N(c2ccccc2)c2cc3c(c4c2oc2ccccc24)-c2c(cc(N(c4ccccc4)c4ccccc4)c4c2oc2ccccc24)C32c3ccccc3-c3ccccc32)cc1 ; c1ccc(N(c2ccc3ccccc3c2)c2cc3c(c4c2oc2ccccc24)-c2c(cc(N(c4ccccc4)c4ccccc4)c4c2oc2ccccc24)C32c3ccccc3-c3ccccc32)cc1]

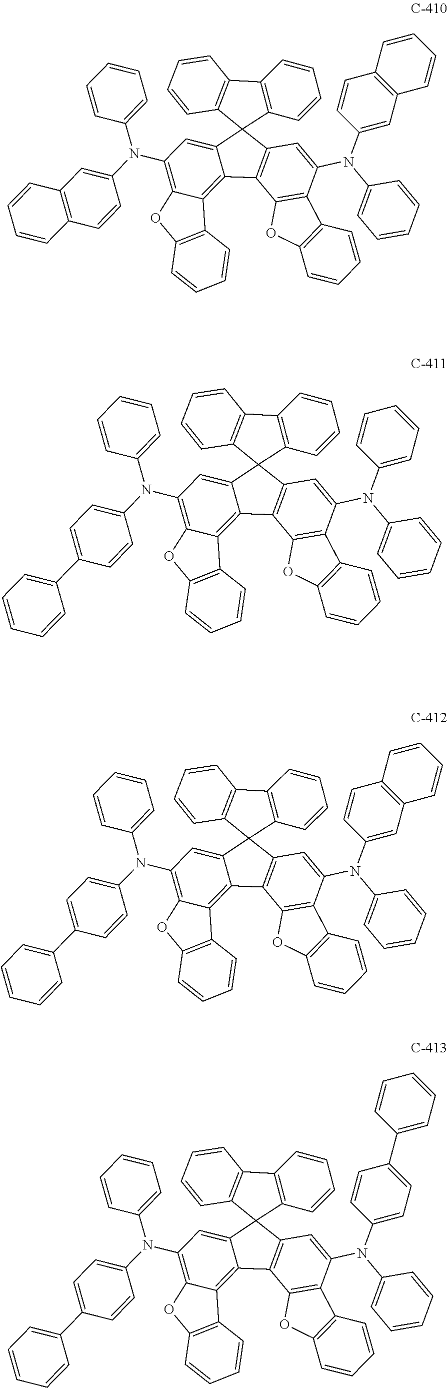 [SMILES: c1ccc(N(c2ccc3ccccc3c2)c2cc3c(c4c2oc2ccccc24)-c2c(cc(N(c4ccccc4)c4ccc5ccccc5c4)c4c2oc2ccccc24)C32c3ccccc3-c3ccccc32)cc1 ; c1ccc(-c2ccc(N(c3ccccc3)c3cc4c(c5c3oc3ccccc35)-c3c(cc(N(c5ccccc5)c5ccccc5)c5c3oc3ccccc35)C43c4ccccc4-c4ccccc43)cc2)cc1 ; c1ccc(-c2ccc(N(c3ccccc3)c3cc4c(c5c3oc3ccccc35)-c3c(cc(N(c5ccccc5)c5ccc6ccccc6c5)c5c3oc3ccccc35)C43c4ccccc4-c4ccccc43)cc2)cc1 ; c1ccc(-c2ccc(N(c3ccccc3)c3cc4c(c5c3oc3ccccc35)-c3c(cc(N(c5ccccc5)c5ccc(-c6ccccc6)cc5)c5c3oc3ccccc35)C43c4ccccc4-c4ccccc43)cc2)cc1]

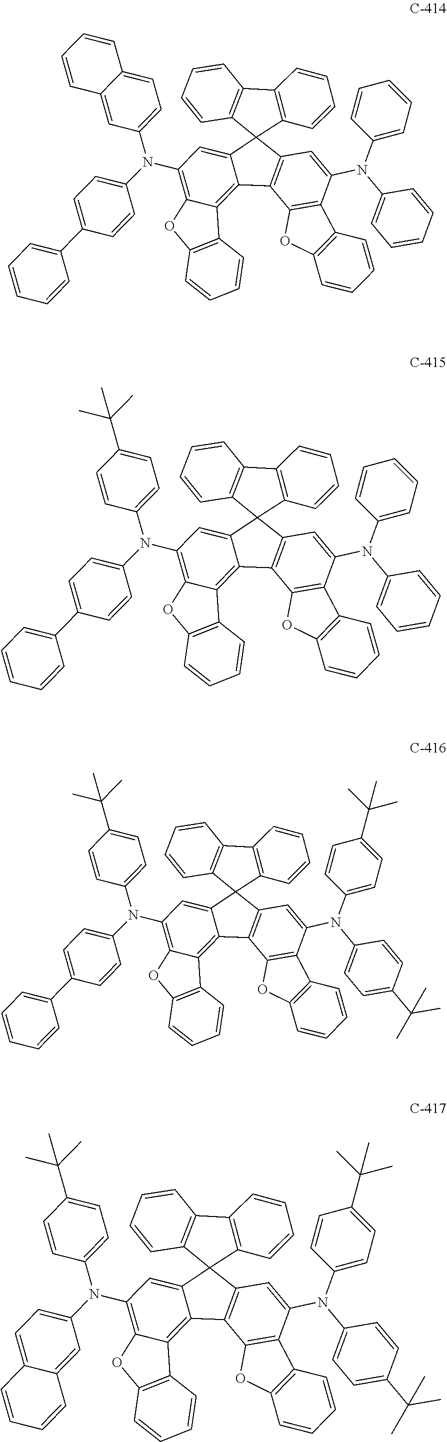

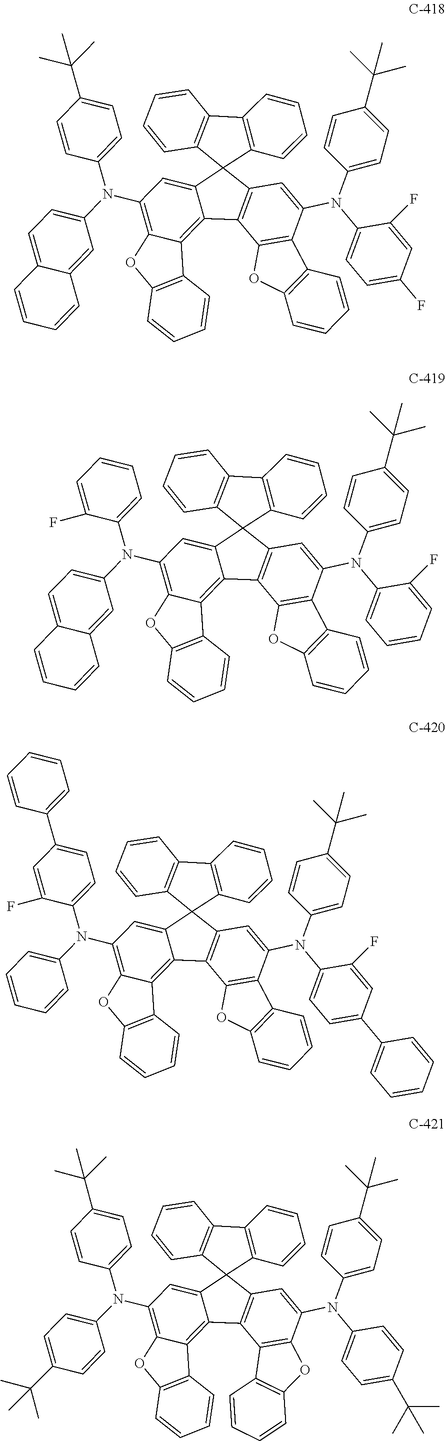

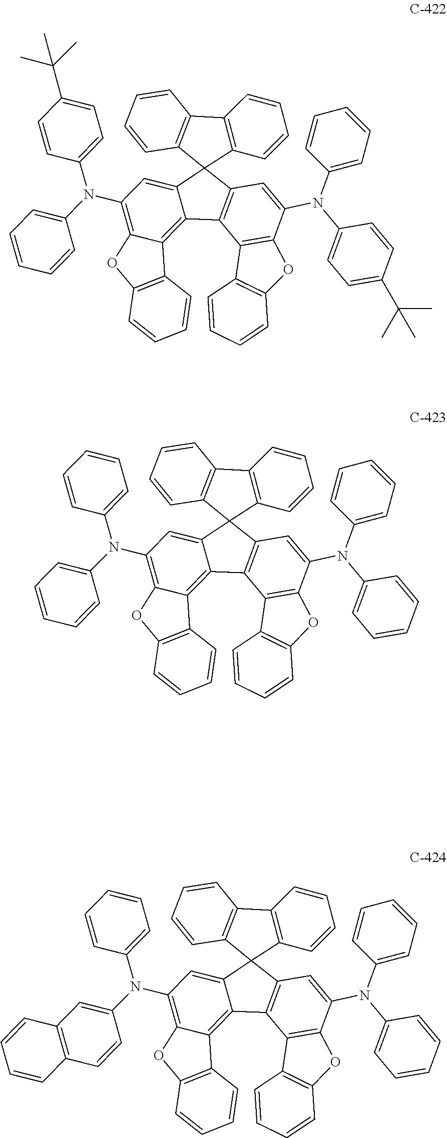 [SMILES: CC(C)(C)c1ccc(N(c2ccccc2)c2cc3c(c4c2oc2ccccc24)-c2c(cc(N(c4ccccc4)c4ccc(C(C)(C)C)cc4)c4oc5ccccc5c24)C32c3ccccc3-c3ccccc32)cc1 ; c1ccc(N(c2ccccc2)c2cc3c(c4c2oc2ccccc24)-c2c(cc(N(c4ccccc4)c4ccccc4)c4oc5ccccc5c24)C32c3ccccc3-c3ccccc32)cc1 ; c1ccc(N(c2ccccc2)c2cc3c(c4c2oc2ccccc24)-c2c(cc(N(c4ccccc4)c4ccc5ccccc5c4)c4oc5ccccc5c24)C32c3ccccc3-c3ccccc32)cc1]

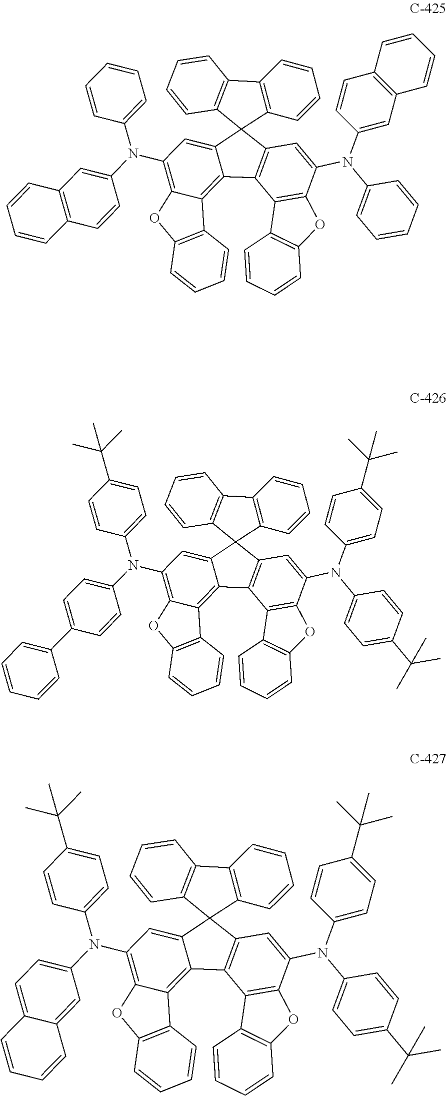 [SMILES: c1ccc(N(c2ccc3ccccc3c2)c2cc3c(c4c2oc2ccccc24)-c2c(cc(N(c4ccccc4)c4ccc5ccccc5c4)c4oc5ccccc5c24)C32c3ccccc3-c3ccccc32)cc1 ; CC(C)(C)c1ccc(N(c2ccc(-c3ccccc3)cc2)c2cc3c(c4c2oc2ccccc24)-c2c(cc(N(c4ccc(C(C)(C)C)cc4)c4ccc(C(C)(C)C)cc4)c4oc5ccccc5c24)C32c3ccccc3-c3ccccc32)cc1 ; CC(C)(C)c1ccc(N(c2ccc(C(C)(C)C)cc2)c2cc3c(c4c2oc2ccccc24)-c2c(cc(N(c4ccc(C(C)(C)C)cc4)c4ccc5ccccc5c4)c4oc5ccccc5c24)C32c3ccccc3-c3ccccc32)cc1]

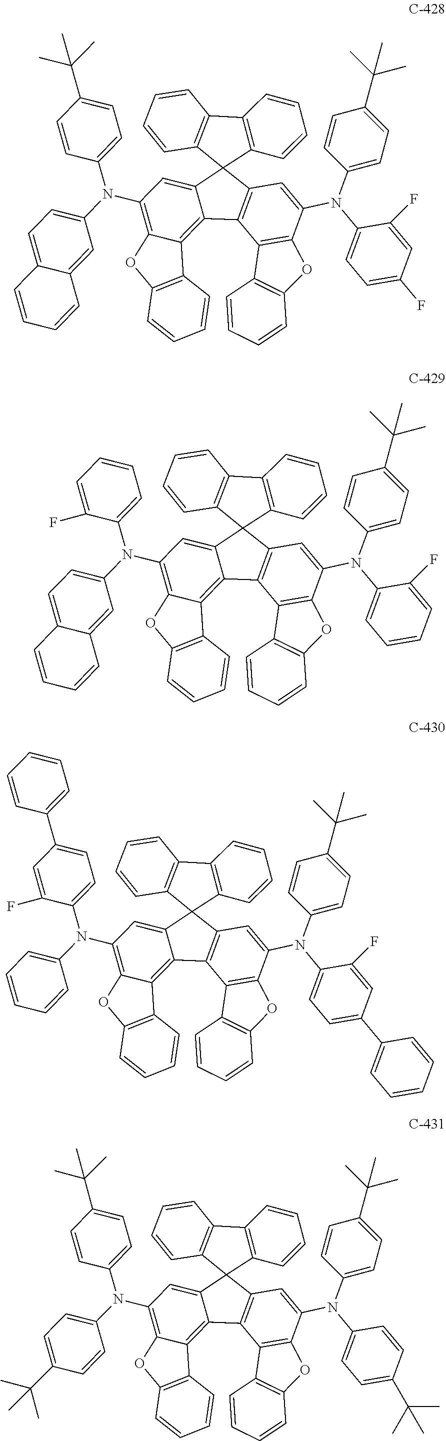

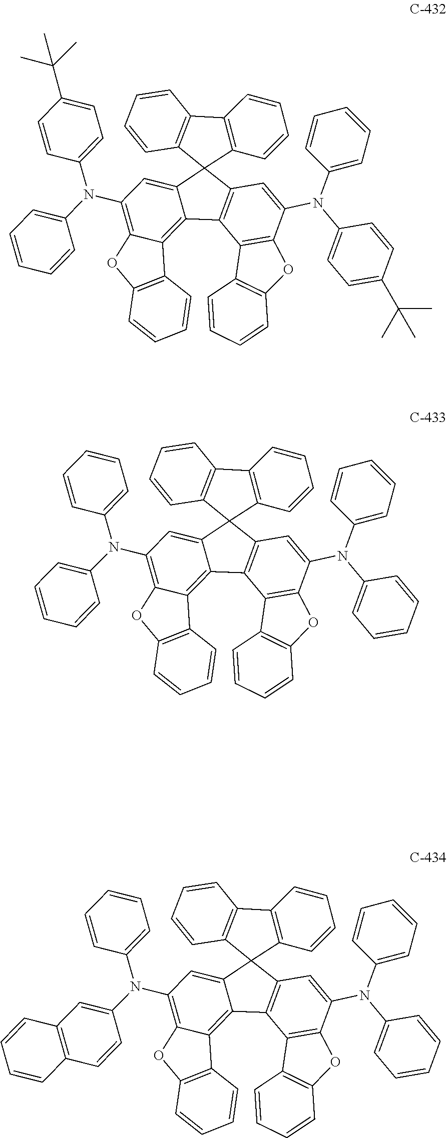 [SMILES: CC(C)(C)c1ccc(N(c2ccccc2)c2cc3c(c4c2oc2ccccc24)-c2c(cc(N(c4ccccc4)c4ccc(C(C)(C)C)cc4)c4oc5ccccc5c24)C32c3ccccc3-c3ccccc32)cc1 ; c1ccc(N(c2ccccc2)c2cc3c(c4c2oc2ccccc24)-c2c(cc(N(c4ccccc4)c4ccccc4)c4oc5ccccc5c24)C32c3ccccc3-c3ccccc32)cc1 ; c1ccc(N(c2ccccc2)c2cc3c(c4c2oc2ccccc24)-c2c(cc(N(c4ccccc4)c4ccc5ccccc5c4)c4oc5ccccc5c24)C32c3ccccc3-c3ccccc32)cc1]

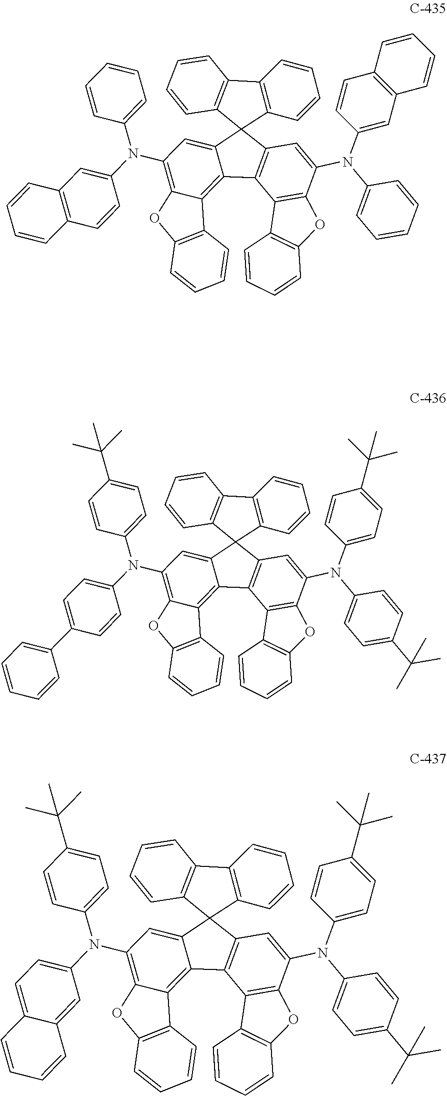 [SMILES: c1ccc(N(c2ccc3ccccc3c2)c2cc3c(c4c2oc2ccccc24)-c2c(cc(N(c4ccccc4)c4ccc5ccccc5c4)c4oc5ccccc5c24)C32c3ccccc3-c3ccccc32)cc1 ; CC(C)(C)c1ccc(N(c2ccc(-c3ccccc3)cc2)c2cc3c(c4c2oc2ccccc24)-c2c(cc(N(c4ccc(C(C)(C)C)cc4)c4ccc(C(C)(C)C)cc4)c4oc5ccccc5c24)C32c3ccccc3-c3ccccc32)cc1 ; CC(C)(C)c1ccc(N(c2ccc(C(C)(C)C)cc2)c2cc3c(c4c2oc2ccccc24)-c2c(cc(N(c4ccc(C(C)(C)C)cc4)c4ccc5ccccc5c4)c4oc5ccccc5c24)C32c3ccccc3-c3ccccc32)cc1]

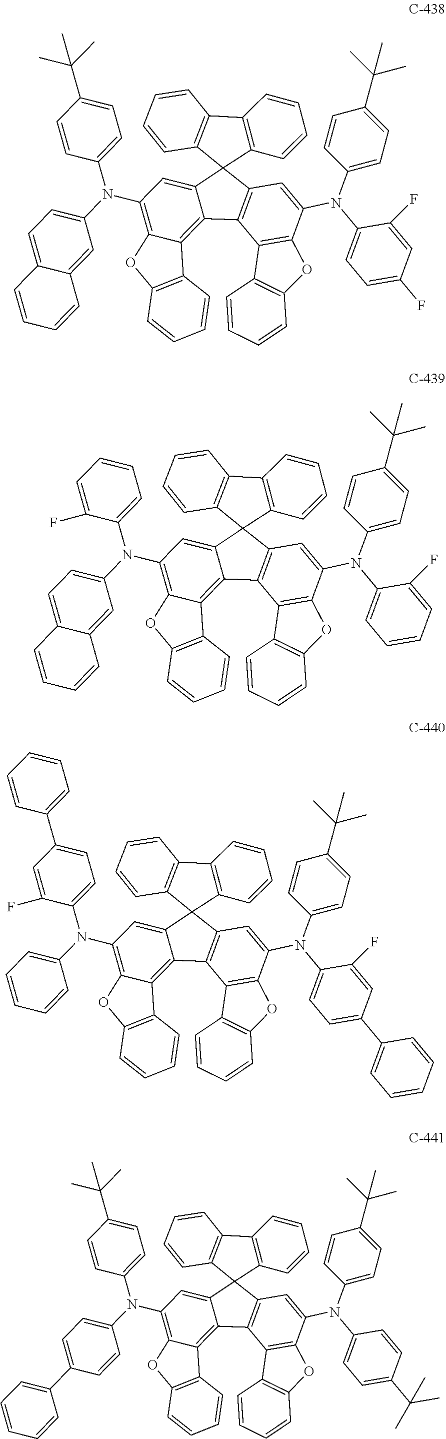

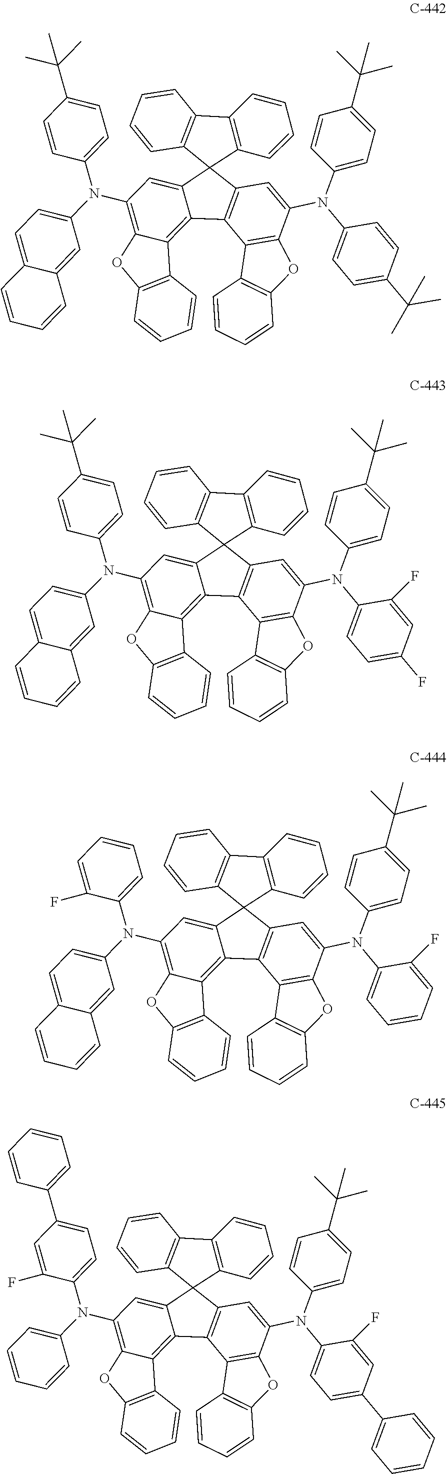

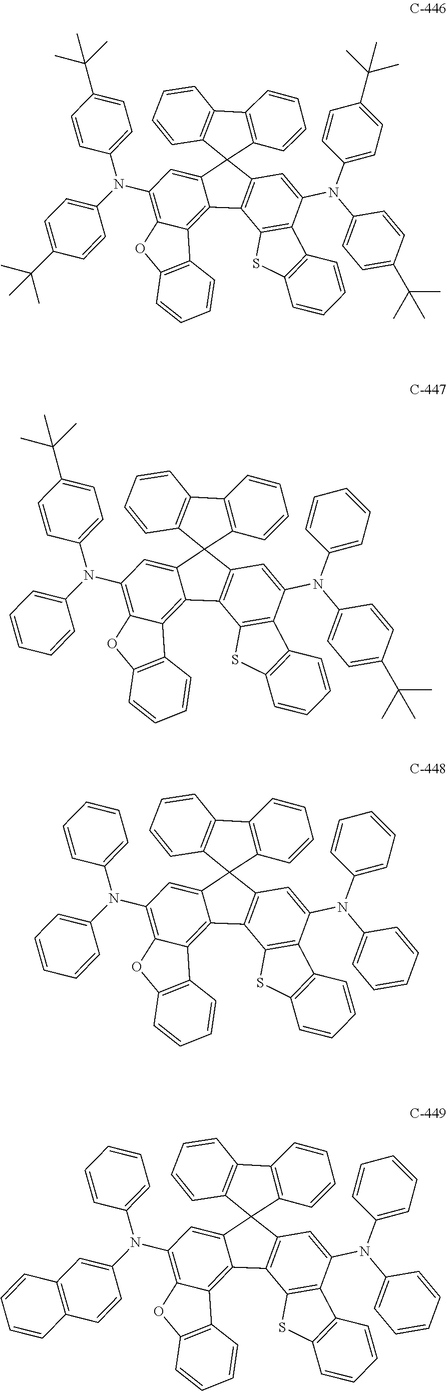 [SMILES: CC(C)(C)c1ccc(N(c2ccc(C(C)(C)C)cc2)c2cc3c(c4c2oc2ccccc24)-c2c(cc(N(c4ccc(C(C)(C)C)cc4)c4ccc(C(C)(C)C)cc4)c4c2sc2ccccc24)C32c3ccccc3-c3ccccc32)cc1 ; CC(C)(C)c1ccc(N(c2ccccc2)c2cc3c(c4c2oc2ccccc24)-c2c(cc(N(c4ccccc4)c4ccc(C(C)(C)C)cc4)c4c2sc2ccccc24)C32c3ccccc3-c3ccccc32)cc1 ; c1ccc(N(c2ccccc2)c2cc3c(c4c2oc2ccccc24)-c2c(cc(N(c4ccccc4)c4ccccc4)c4c2sc2ccccc24)C32c3ccccc3-c3ccccc32)cc1 ; c1ccc(N(c2ccc3ccccc3c2)c2cc3c(c4c2oc2ccccc24)-c2c(cc(N(c4ccccc4)c4ccccc4)c4c2sc2ccccc24)C32c3ccccc3-c3ccccc32)cc1]

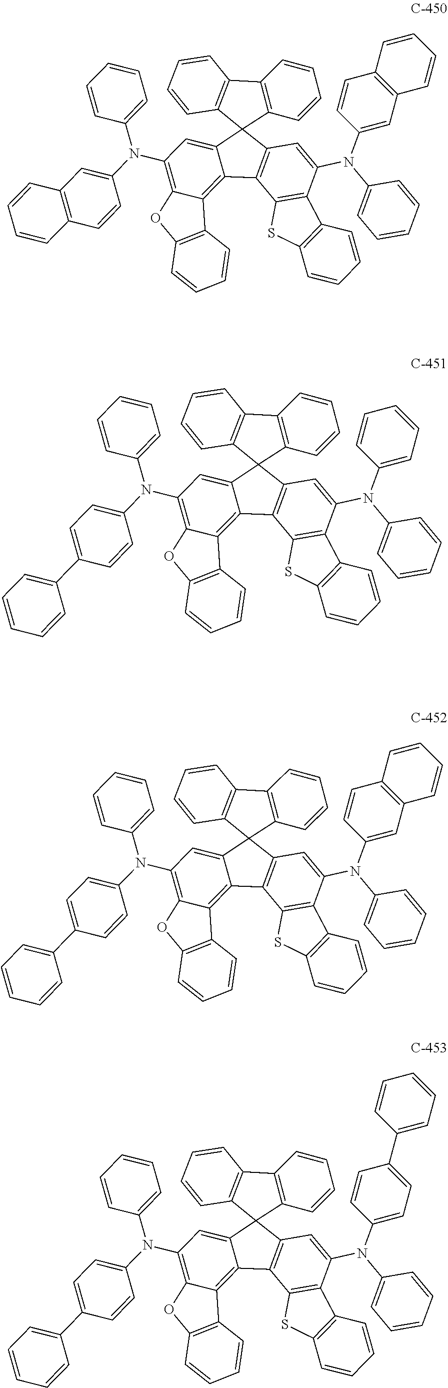 [SMILES: c1ccc(N(c2ccc3ccccc3c2)c2cc3c(c4c2oc2ccccc24)-c2c(cc(N(c4ccccc4)c4ccc5ccccc5c4)c4c2sc2ccccc24)C32c3ccccc3-c3ccccc32)cc1 ; c1ccc(-c2ccc(N(c3ccccc3)c3cc4c(c5c3oc3ccccc35)-c3c(cc(N(c5ccccc5)c5ccccc5)c5c3sc3ccccc35)C43c4ccccc4-c4ccccc43)cc2)cc1 ; c1ccc(-c2ccc(N(c3ccccc3)c3cc4c(c5c3oc3ccccc35)-c3c(cc(N(c5ccccc5)c5ccc6ccccc6c5)c5c3sc3ccccc35)C43c4ccccc4-c4ccccc43)cc2)cc1 ; c1ccc(-c2ccc(N(c3ccccc3)c3cc4c(c5c3oc3ccccc35)-c3c(cc(N(c5ccccc5)c5ccc(-c6ccccc6)cc5)c5c3sc3ccccc35)C43c4ccccc4-c4ccccc43)cc2)cc1]

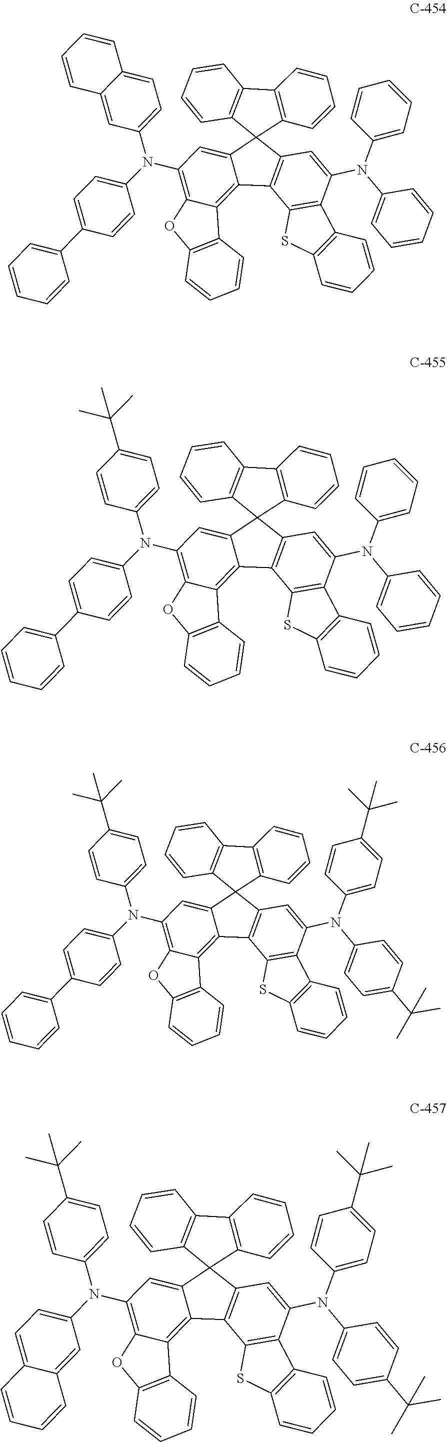

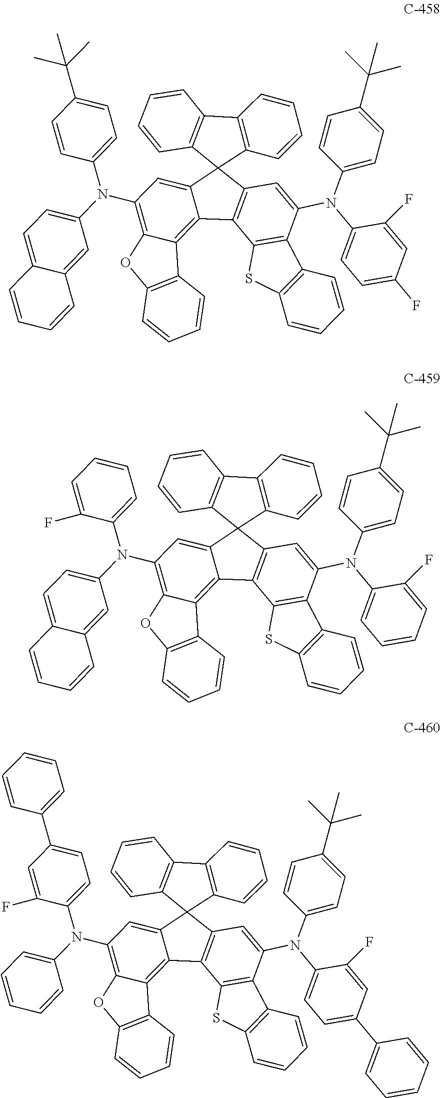 [SMILES: CC(C)(C)c1ccc(N(c2ccc3ccccc3c2)c2cc3c(c4c2oc2ccccc24)-c2c(cc(N(c4ccc(C(C)(C)C)cc4)c4ccc(F)cc4F)c4c2sc2ccccc24)C32c3ccccc3-c3ccccc32)cc1 ; CC(C)(C)c1ccc(N(c2ccccc2F)c2cc3c(c4sc5ccccc5c24)-c2c(cc(N(c4ccc5ccccc5c4)c4ccccc4F)c4oc5ccccc5c24)C32c3ccccc3-c3ccccc32)cc1 ; CC(C)(C)c1ccc(N(c2ccc(-c3ccccc3)cc2F)c2cc3c(c4sc5ccccc5c24)-c2c(cc(N(c4ccccc4)c4ccc(-c5ccccc5)cc4F)c4oc5ccccc5c24)C32c3ccccc3-c3ccccc32)cc1]

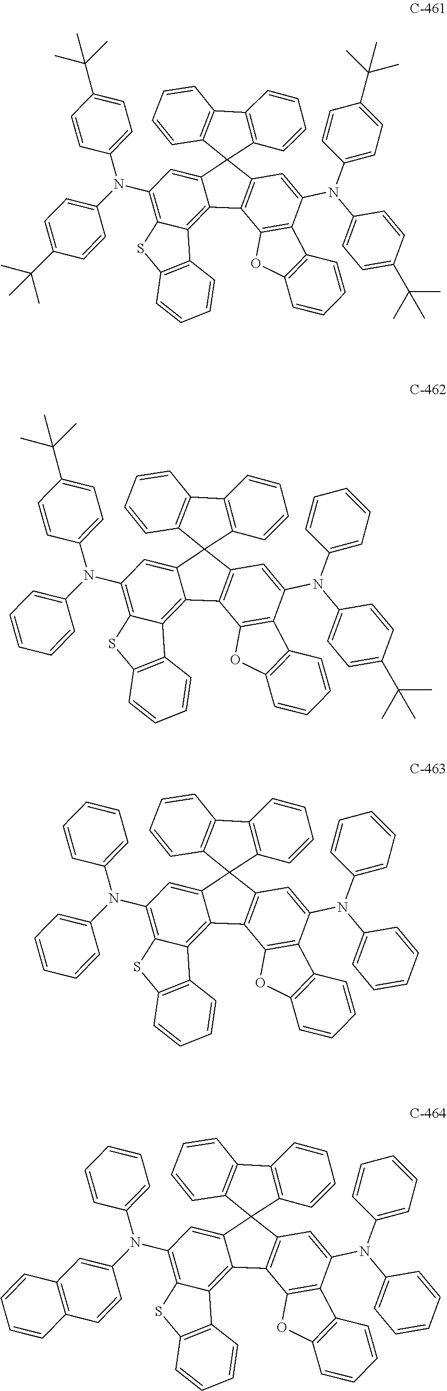 [SMILES: CC(C)(C)c1ccc(N(c2ccc(C(C)(C)C)cc2)c2cc3c(c4c2sc2ccccc24)-c2c(cc(N(c4ccc(C(C)(C)C)cc4)c4ccc(C(C)(C)C)cc4)c4c2oc2ccccc24)C32c3ccccc3-c3ccccc32)cc1 ; CC(C)(C)c1ccc(N(c2ccccc2)c2cc3c(c4c2sc2ccccc24)-c2c(cc(N(c4ccccc4)c4ccc(C(C)(C)C)cc4)c4c2oc2ccccc24)C32c3ccccc3-c3ccccc32)cc1 ; c1ccc(N(c2ccccc2)c2cc3c(c4c2sc2ccccc24)-c2c(cc(N(c4ccccc4)c4ccccc4)c4c2oc2ccccc24)C32c3ccccc3-c3ccccc32)cc1 ; c1ccc(N(c2ccc3ccccc3c2)c2cc3c(c4c2sc2ccccc24)-c2c(cc(N(c4ccccc4)c4ccccc4)c4c2oc2ccccc24)C32c3ccccc3-c3ccccc32)cc1]

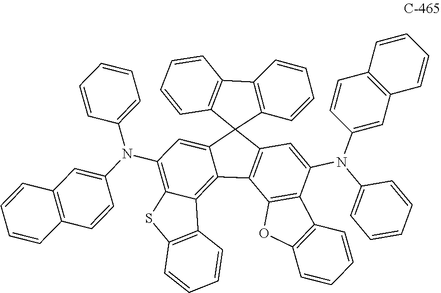 [SMILES: c1ccc(N(c2ccc3ccccc3c2)c2cc3c(c4c2sc2ccccc24)-c2c(cc(N(c4ccccc4)c4ccc5ccccc5c4)c4c2oc2ccccc24)C32c3ccccc3-c3ccccc32)cc1]

The organic electroluminescent devices 10 and 20 of the present disclosure may be prepared by forming the first electrode 110 or the second electrode 410 on a substrate; forming a light-emitting units 200 and 300 by using any one method of dry film-forming methods such as vacuum deposition, sputtering, plasma, or ion plating, or wet film-forming methods such as ink jet printing, nozzle printing, slot coating, spin coating, dip coating, or flow coating: and then forming the charge generation layer 500 and the second electrode 410 or the first electrode 110 thereon.

When using a wet film-forming method, a thin film may be formed by dissolving or diffusing materials forming each layer into any suitable solvent such as ethanol, chloroform, tetrahydrofuran, dioxane, etc. The solvent may be any solvent where the materials forming each layer can be dissolved or diffused, and where there are no problems in film-formation capability.

According to one embodiment, the present disclosure can provide display devices comprising a deuterated compound represented by Formulas 1-1 to 1-4 and/or Formulas 2 and 2-1 In addition, by using the organic electroluminescent device of the present disclosure, display devices such as smartphones, tablets, notebooks, PCs, TVs, or display devices for vehicles, or lighting devices such as outdoor or indoor lighting can be prepared.

Hereinafter, the preparation method of the compounds according to the present disclosure will be explained with reference to the synthesis method of a representative compound or intermediate compound in order to understand the present disclosure in detail.

### [Example 1] Synthesis of Compound N-164

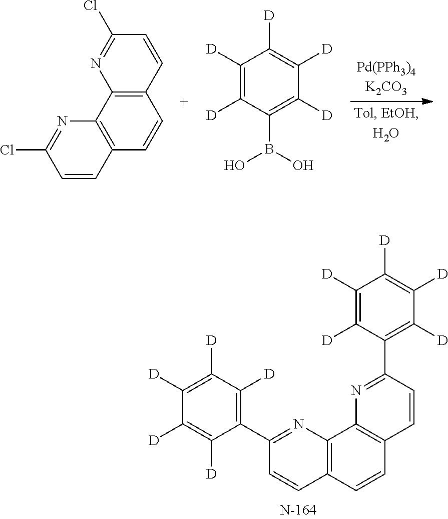 [SMILES: Clc1ccc2ccc3ccc(Cl)nc3c2n1 ; [2H]c1c([2H])c([2H])c(B(O)O)c([2H])c1[2H] ; [2H]c1c([2H])c([2H])c(-c2ccc3ccc4ccc(-c5c([2H])c([2H])c([2H])c([2H])c5[2H])nc4c3n2)c([2H])c1[2H]]

2,9-Dichloro-1,10-phenanthroline (10.0 g, 40 mmol), (phenyl-Ds)boronic acid (11.2 g, 88 mmol), Pd(PPh₃)₄ (2.3 g, 2.0 mmol), and K₂CO₃ (11.1 g, 80 mmol) were added to 120 mL of toluene, 40 mL of distilled water, and 40 mL of ethanol, and stirred under reflux at 150° C. After 17 hours, the mixture was cooled to room temperature, distilled water was added thereto, and the organic layer was extracted with ethyl acetate. Afterwards, the residual moisture was removed using magnesium sulfate, distilled under reduced pressure, and separated by column chromatography to obtain Compound N-164 (10 g, yield: 73.0%).

### [Example 2] Synthesis of Compound N-2

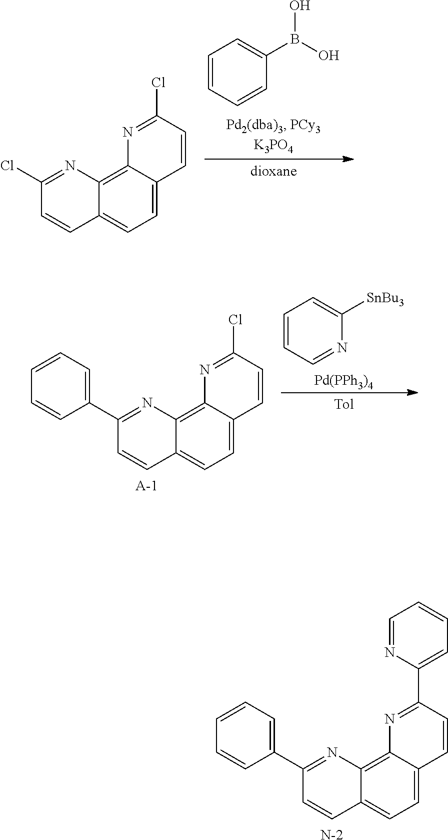

1) Synthesis of Compound A-1

2,9-Dichloro-1,10-phenanthroline (15.5 g, 62 mmol), phenylboronic acid (5.3 g, 44 mmol), Pd₂(dba)₃ (1.1 g, 1.2 mmol), PCys (1.5 g, 4.8 mmol), K₃PO₄ (15.7 g, 74 mmol) were added to 310 mL of dioxane, and 31 mL of distilled water, and stirred under reflux at 110° C. After 16 hours, the mixture was cooled to room temperature, distilled water was added thereto, and the organic layer was extracted with ethyl acetate. Afterwards, the residual moisture was removed using magnesium sulfate, distilled under reduced pressure, and separated by column chromatography to obtain Compound A-1 (7.2 g, yield: 40%).

2) Synthesis of Compound N-2

Compound A-1 (7.2 g, 25 mmol) 2-(tributylstannyl)pyridine (11 g, 30 mmol), and Pd(PPh₃)₄ (1.5 g, 1.3 mmol) were added to 140 mL of toluene, and stirred under reflux at 150° C. After 16 hours, the mixture was cooled to room temperature, distilled under reduced pressure through a celite filter, and then separated by column chromatography to obtain Compound N-2 (4.0 g, yield: 48%).

### [Example 3] Synthesis of Compound H1-1

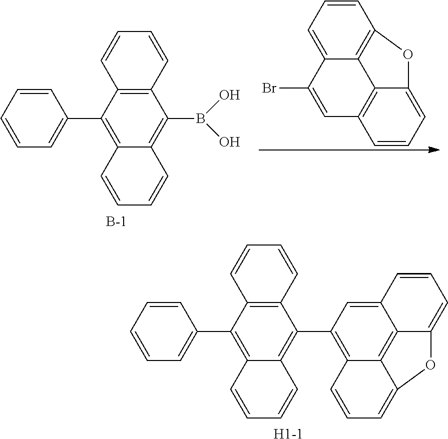

Compound B-1 (12.6 g, 42.41 mmol), 8-bromophenanthro[4,5-bcd]furan (10.0 g, 36.89 mmol), PdCl₂(AMPHOS)₂ (1.27 g, 1.84 mmol), Na₂CO₃ (7.8 g, 73.77 mmol), Aliquat® 336 (0.74 g, 1.84 mmol), 150 ml of toluene, and 50 mL of distilled water were added to a flask, and stirred under reflux at 150° C. After 3 hours, the mixture was cooled to room temperature, distilled water was added thereto, and the organic layer was extracted with ethyl acetate. Afterwards, the residual moisture was removed using magnesium sulfate, distilled under reduced pressure, and separated by column chromatography to obtain Compound H1-1 (7 g, yield: 42.68%).

### [Example 4] Synthesis of Compound H1-226

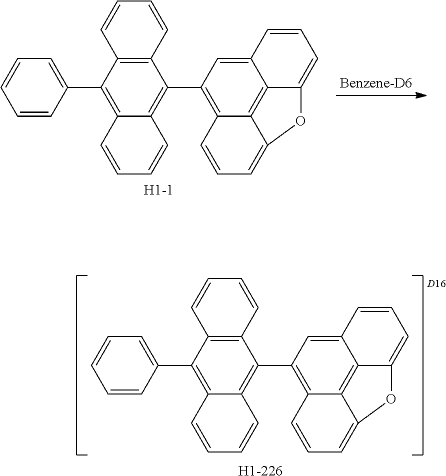

Compound H1-1 (1 g, 2.25 mmol) and benzene-D6 (25 mL, 280.37 mmol) were added to a flask, dissolved by heating the mixture, and then added triflic acid (0.3 mL, 3.39 mmol) thereto at 60° C. After 2 hours, the mixture was cooled to room temperature, 1 mL of D₂O was added thereto and stirred for 10 minutes. After the reaction was completed, the mixture was neutralized with an aqueous K₃PO₄ solution, the organic layer was extracted with ethyl acetate, the residual moisture was removed using magnesium sulfate, and the mixture was distilled under reduced pressure. Afterwards, it was separated by column chromatography to obtain Compound H1-226 (0.6 g, yield: 57.91%).

### [Example 5] Synthesis of Compound N-216

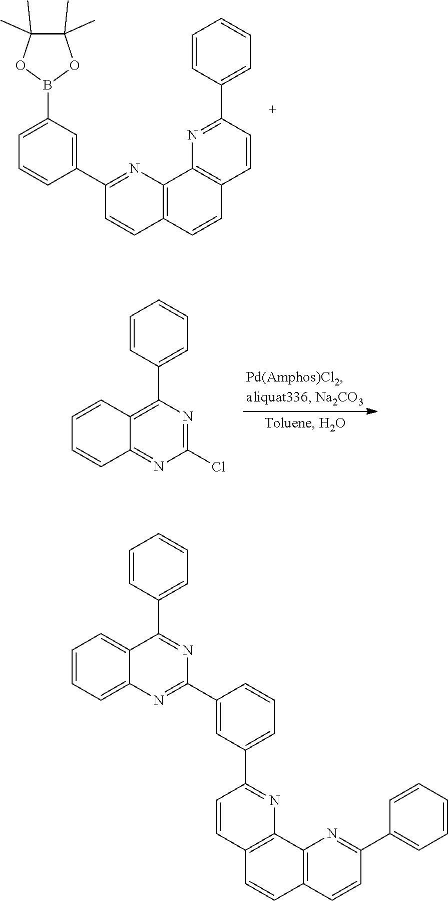 [SMILES: CC1(C)OB(c2cccc(-c3ccc4ccc5ccc(-c6ccccc6)nc5c4n3)c2)OC1(C)C ; Clc1nc(-c2ccccc2)c2ccccc2n1 ; c1ccc(-c2ccc3ccc4ccc(-c5cccc(-c6nc(-c7ccccc7)c7ccccc7n6)c5)nc4c3n2)cc1]

2-phenyl-9-(3-(4,4,5,5-tetramethyl-1,3,2-dioxaborolan-2-yl)phenyl)-1,10-phenanthroline (10.0 g, 21.82 mmol), 2-chloro-4-phenylquinazoline (5.3 g, 21.82 mmol), Pd(Amphos)Cl₂ (1.1 g, 1.53 mmol), Aliquat® 336 (1.8 g, 4.36 mmol), and Na₂CO₃ (4.6 g, 43.64 mmol) were added to 110 mL of toluene, and 36 mL of H₂O and dissolved, and then stirred under reflux at 150° C. for 2 hours. After the reaction was completed, the mixture was cooled to room temperature, the layers were separated, filtered through silica, and then recrystallized to obtain Compound N-216 (4.9 g, yield: 41.84%).

| | MW | M.P |
|---|---|---|
| N-216 | 536.62 | 247° C. |

### [Example 6] Synthesis of Compound N-176

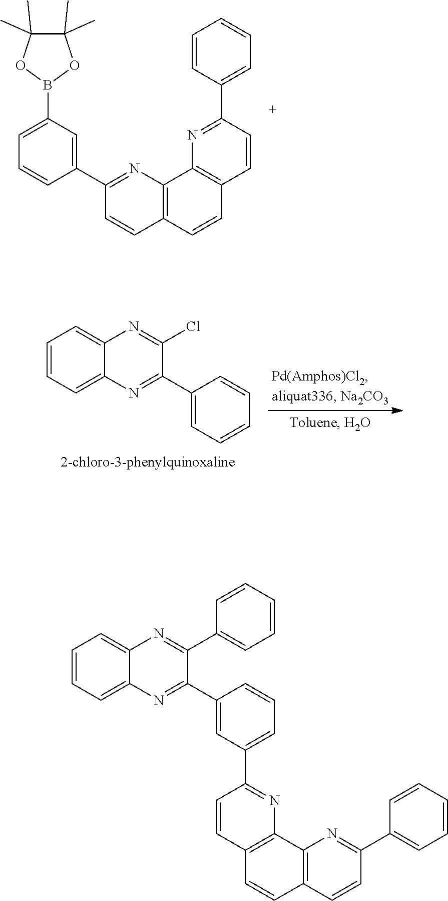 [SMILES: CC1(C)OB(c2cccc(-c3ccc4ccc5ccc(-c6ccccc6)nc5c4n3)c2)OC1(C)C ; Clc1nc2ccccc2nc1-c1ccccc1 ; c1ccc(-c2ccc3ccc4ccc(-c5cccc(-c6nc7ccccc7nc6-c6ccccc6)c5)nc4c3n2)cc1]

2-Phenyl-9-(3-(4,4,5,5-tetramethyl-1,3,2-dioxaborolan-2-yl)phenyl)-1,10-phenanthroline (15.0 g, 32.71 mmol), 2-chloro-3-phenylquinoxaline (8.0 g, 32.71 mmol), Pd(Amphos)Cl₂ (1.6 g, 2.29 mmol), Aliquat 336 (1.3 g, 3.27 mmol), and Na₂CO₃ (7.0 g, 65.42 mmol) were added to 165 mL of toluene and 55 mL of H₂O, and dissolved, and then stirred under reflux at 150° C. for 3 hours. After the reaction was completed, the mixture was cooled to room temperature, the layers were separate into layers, filtered through silica, and then separate by column chromatography to obtain Compound N-176 (4.1 g, yield: 24%) as a solid.

| | MW | M.P |
|---|---|---|
| N-176 | 536.62 | 220° C. |

### [Example 7] Synthesis of Compound N-3

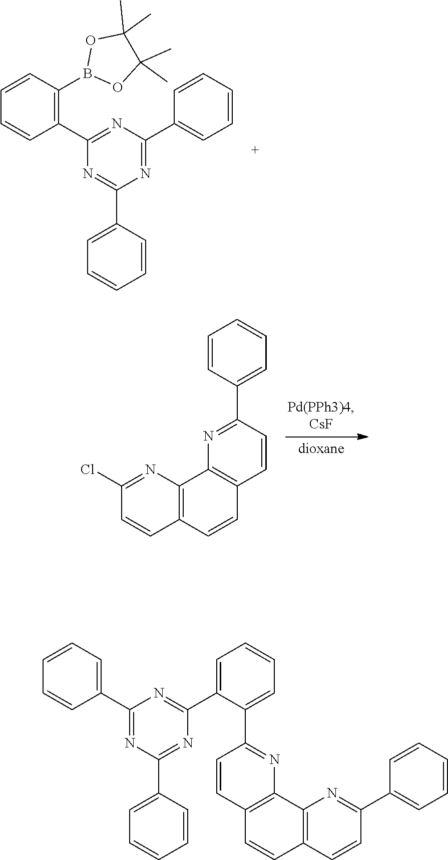 [SMILES: CC1(C)OB(c2ccccc2-c2nc(-c3ccccc3)nc(-c3ccccc3)n2)OC1(C)C ; Clc1ccc2ccc3ccc(-c4ccccc4)nc3c2n1 ; c1ccc(-c2ccc3ccc4ccc(-c5ccccc5-c5nc(-c6ccccc6)nc(-c6ccccc6)n5)nc4c3n2)cc1]

2,4-Diphenyl-6-(2-(4,4,5,5-tetramethyl-1,3,2-dioxaborolan-2-yl)phenyl)-1,3,5-triazine (1.0 g, 2.2 mmol), 2-chloro-9-phenyl-1,10-phenanthroline (0.65 g, 2.2 mmol), Pd(PPh₃)₄ (0.13 g, 0.11 mmol), and CsF (0.67 g, 4.5 mmol) were dissolved in 10 mL of 1,4-dioxane, and stirred under reflux at 100° C. for 16 hours. After the reaction was completed, the mixture was cooled to room temperature, the layers were separate, filtered through silica, and then separate by column chromatography to obtain Compound N-3 (0.86 g, yield: 68%) as a solid.

| | MW | M.P |
|---|---|---|
| N-3 | 563.66 | 230° C. |

### [Example 8] Synthesis of Compound N-16

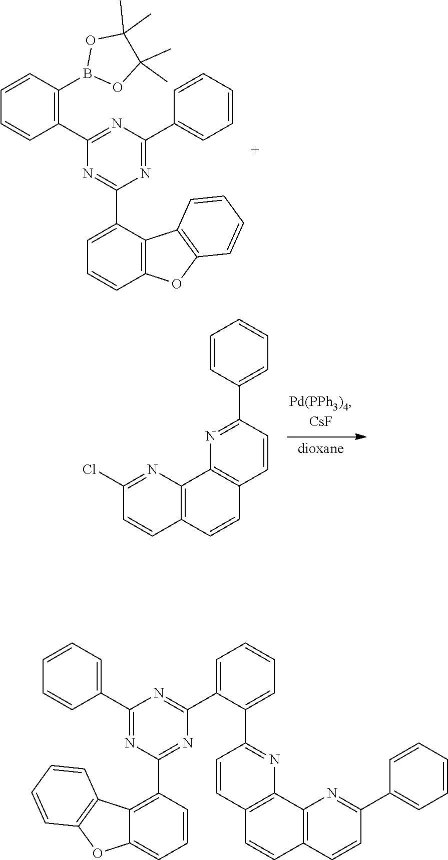 [SMILES: CC1(C)OB(c2ccccc2-c2nc(-c3ccccc3)nc(-c3cccc4oc5ccccc5c34)n2)OC1(C)C ; Clc1ccc2ccc3ccc(-c4ccccc4)nc3c2n1 ; c1ccc(-c2ccc3ccc4ccc(-c5ccccc5-c5nc(-c6ccccc6)nc(-c6cccc7oc8ccccc8c67)n5)nc4c3n2)cc1]

2-(Dibenzo[b,d]furan-1-yl)-4-phenyl-6-(2-(4,4,5,5-tetramethyl-1,3,2-dioxaborolan-2-yl)phenyl)-1,3,5-trazine (2.9 g, 5.5 mmol), 2-chloro-9-phenyl-1,10-phenanthroline (1.5 g, 5 mmol), Pd(PPh₃)₄ (0.58 g, 0.5 mmol), and CsF (1.9 g, 13 mmol) were dissolved in 25 mL of 1,4-dioxane and stirred under reflux at 100° C. for 18 hours. After the reaction was completed, the mixture was cooled to room temperature, the layers were separate, filtered through silica, and then separate by column chromatography to obtain Compound N-16 (2.7 g, yield: 83%) as a solid.

| | MW | M.P |
|---|---|---|
| N-16 | 653.75 | 235° C. |

Hereinafter, the preparation method of an organic electroluminescent device comprising the above-described organic electroluminescent materials, and the device property thereof will be explained in order to understand the present disclosure in detail.

### [Device Examples 1 to 4] Preparation of OLEDs Deposited with a Deuterated Compound According to the Present Disclosure

OLEDs according to the present disclosure were produced. First, a transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and thereafter was stored in isopropyl alcohol and then used. Thereafter, the ITO substrate was mounted on a substrate holder of a vacuum vapor deposition apparatus. Then, Compound HI-1 was introduced into a cell of the vacuum vapor deposition apparatus, and Compound HT-1 was introduced into another cell. The two materials were evaporated at different rates and Compound HI-1 was deposited in a doping amount of 7 wt % based on the total amount of compounds HI-1 and HT-1 to form a hole injection layer having a thickness of 5 nm. Next, Compound HT-1 was deposited as a first hole transport layer having a thickness of 30 nm on the hole injection layer. Next, Compound HT-2 was introduced into another cell of the vacuum vapor deposition apparatus and was evaporated by applying an electric current to the cell, thereby forming a second hole transport layer having a thickness of 5 nm on the first hole transport layer. After forming the hole injection layer and the hole transport layers, a first light-emitting layer was formed thereon as follows: the Compound described in the following Tables 1 and 2 was introduced into two cells of the vacuum vapor deposition apparatus as a host, and Compound C-286 was introduced into another cell as a dopant. The two materials were evaporated at a different rate, and the dopant was deposited in a doping amount of 2 wt % based on the total amount of the host and dopant to form a first light-emitting layer having a thickness of 20 nm on the second hole transport layer. Next, a 5 nm thick hole blocking layer using Compound ET-1 as a first hole blocking layer material was deposited on the first light-emitting layer. Next, compounds ET-2 and EI-1 as electron transport materials were deposited at a weight ratio of 2:1 to form a first electron transport layer having a thickness of 25 nm. Thereafter, an N-type charge generation layer of 10 nm was formed by depositing 1 wt % of Li on the compound shown in Tables 1 and 2 below. Next, Compound HI-1 was doped in an amount of 15 wt % based on the total amount of Compounds HI-1 and HT-1, and a P-type charge generation layer with a thickness of 5 nm was deposited. Next, Compound HT-1 was deposited to form a third hole transport layer having a thickness of 30 nm, and then Compound HT-2 was deposited to form a fourth hole transport layer having a thickness of 5 nm. A second light-emitting layer was formed thereon as follows: the Compound described in the following Tables 1 and 2 was introduced into cell of the vacuum vapor deposition apparatus as a host, and Compound C-286 was introduced into another cell as a dopant. The two materials were evaporated at a different rate, and the dopant was deposited in a doping amount of 2 wt % based on the total amount of the host and dopant to form a second light-emitting layer having a thickness of 20 nm on the fourth hole transport layer. Next, a 5 nm thick hole blocking layer using Compound ET-1 as a second hole blocking layer material was deposited on the second light-emitting layer. Compounds ET-2 and EI-1 were respectively added to two cells in the vacuum vapor deposition apparatus as a second electron transport layer material, and then the two materials were deposited to a thickness of 25 nm at a weight ratio of 2:1. Next, Yb was deposited as an electron injection layer to a thickness of 1 nm on the second electron transport layer, and then an Al cathode was deposited to a thickness of 80 nm on the electron injection layer using another vacuum vapor deposition apparatus. Thus, OLEDs were produced. Each compound used for all the materials were purified by vacuum sublimation under 10⁻⁶ torr.

### [Device Comparative Examples 1 and 2] Preparation of OLEDs not Comprising a Deuterated Compound

OLEDs were manufactured in the same manner as Device Example I above, except that the compounds shown in Tables 1 and 2 below were used as the host material of the first light-emitting layer and the N-type charge generation layer material, respectively.

The driving voltage, luminous efficiency, the time taken for luminance to decrease from 100% to 95% (lifespan: T95), and progressive driving voltage change (ΔV) at a luminance of 1000 nits of the OLEDs of Device Examples 1 to 4 and Device Comparative Examples 1 and 2 produced as described above, were measured, and the results thereof are shown in the following Tables and 2.

TABLE 1

| | First | Second | N-Type | | | | |
|---|---|---|---|---|---|---|---|
| | Light- | Light- | Charge | Driving | Luminous | | |
| | emitting | emitting | Generation | Voltage | Efficiency | Lifespan | ΔV |
| | Layer | Layer | Layer | [V] | [cd/A] | [T95, hr] | (%) |
| Device | H-66- | H-11 | N-74 | 7.1 | 11.1 | 130.3 | 101.7 |
| Example 1 | D24 | | | | | | |
| Device | H-66- | H-11 | N-164- | 7.1 | 11.0 | 146.9 | 101.6 |
| Example 2 | D24 | | D10 | | | | |
| Device | H-11 | H-11 | N-74 | 7.1 | 11.1 | 107.5 | 101.8 |
| Comparative | | | | | | | |
| Example 1 | | | | | | | |

TABLE 2

| | First | Second | N-Type | | | | |
|---|---|---|---|---|---|---|---|
| | Light- | Light- | Charge | Driving | Luminous | | |
| | emitting | emitting | Generation | Voltage | Efficiency | Lifespan | ΔV |
| | Layer | Layer | Layer | [V] | [cd/A] | [T95, hr] | [%) |
| Device | H1-1 | H1-1 | N-164-D10 | 6.4 | 8.7 | 408.7 | 101.8 |
| Example 3 | | | | | | | |
| Device | H1-226-D16 | H1-1 | N-164-D10 | 6.5 | 8.8 | 533.0 | 101.9 |
| Example 4 | | | | | | | |
| Device | H1-1 | H1-1 | N-74 | 6.4 | 8.8 | 399.5 | 101.9 |
| Comparative | | | | | | | |
| Example 2 | | | | | | | |

From Tables 1 and 2 above, it can be confirmed that the organic electroluminescent device containing the deuterated compound according to the present disclosure as materials for the host of the light-emitting layer and/or N-type charge generation layer exhibits improved progressive driving voltage characteristics and significantly improved lifespan characteristics.

The compounds used in Device Examples 1 to 4 and Device Comparative Examples 1 and 2 above are shown in the following Table 3.

TABLE 3

| | |
|---|---|
| Hole Injection Layer/Hole Transport Layer | 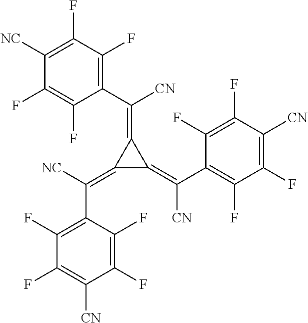 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F] |
| | HI-1 |
| | 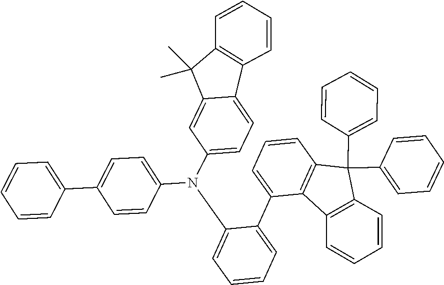 [SMILES: CC1(C)c2ccccc2-c2ccc(N(c3ccc(-c4ccccc4)cc3)c3ccccc3-c3cccc4c3-c3ccccc3C4(c3ccccc3)c3ccccc3)cc21] |
| | HT-1 |
| | 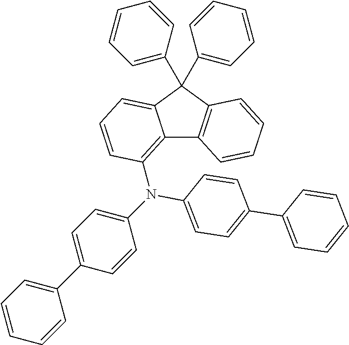 [SMILES: c1ccc(-c2ccc(N(c3ccc(-c4ccccc4)cc3)c3cccc4c3-c3ccccc3C4(c3ccccc3)c3ccccc3)cc2)cc1] |
| | HT-2 |
| Light-Emitting Layer | 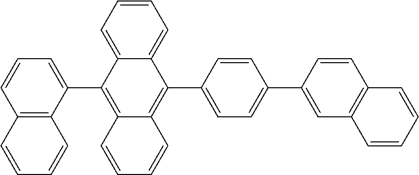 [SMILES: c1ccc2cc(-c3ccc(-c4c5ccccc5c(-c5cccc6ccccc56)c5ccccc45)cc3)ccc2c1] |
| | H-11 |
| | 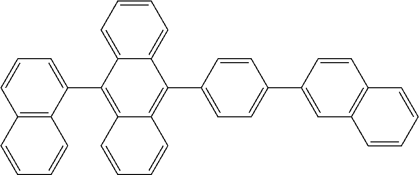 [SMILES: c1ccc2cc(-c3ccc(-c4c5ccccc5c(-c5cccc6ccccc56)c5ccccc45)cc3)ccc2c1] |
| | H-66-D24 |
| | 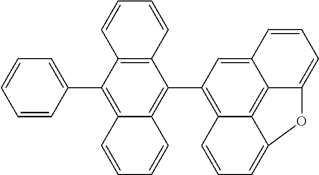 [SMILES: c1ccc(-c2c3ccccc3c(-c3cc4cccc5oc6cccc3c6c45)c3ccccc23)cc1] |
| | H1-1 |
| | 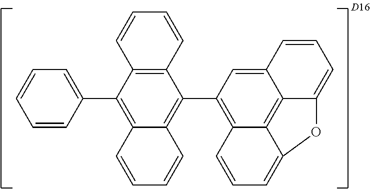 |
| | H1-226-D16 |
| | 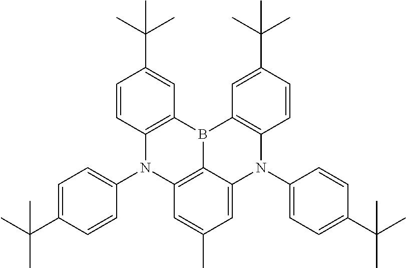 [SMILES: Cc1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1] |
| | C-286 |
| Electron Transport Layer/Hole Blocking Layer/Electron Injection Layer | 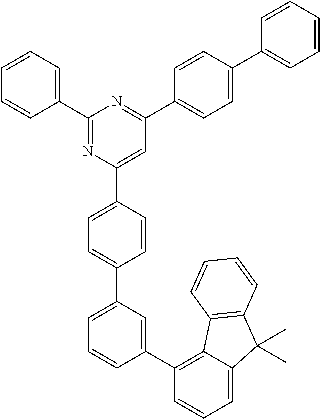 [SMILES: CC1(C)c2ccccc2-c2c(-c3cccc(-c4ccc(-c5cc(-c6ccc(-c7ccccc7)cc6)nc(-c6ccccc6)n5)cc4)c3)cccc21] |
| | ET-1 |
| | 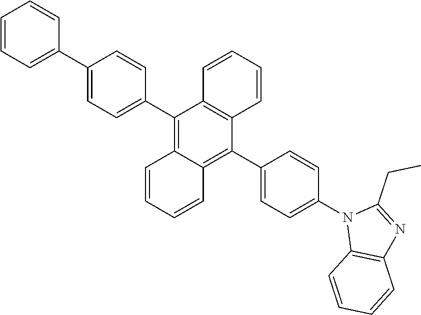 [SMILES: CCc1nc2ccccc2n1-c1ccc(-c2c3ccccc3c(-c3ccc(-c4ccccc4)cc3)c3ccccc23)cc1] |
| | ET-2 |
| | 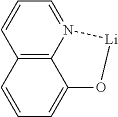 |
| | EI-1 |
| N-type Charge Generation Layer | 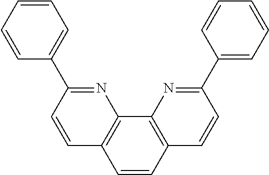 [SMILES: c1ccc(-c2ccc3ccc4ccc(-c5ccccc5)nc4c3n2)cc1] |
| | N-74 |
| | 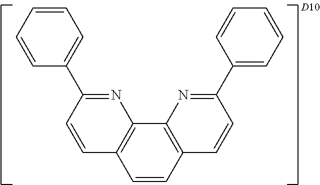 |
| | N-164-D10 |

### [Device Example 5] Preparation of an OLED Comprising the Compound According to the Present Disclosure as a Hole Blocking Layer Material

An OLED according to the present disclosure was produced. First, a transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and thereafter was stored in isopropyl alcohol and then used. Thereafter, the ITO substrate was mounted on a substrate holder of a vacuum vapor deposition apparatus. Then, Compound HI-1 was introduced into a cell of the vacuum vapor deposition apparatus, and Compound HT-1 was introduced into another cell. The two materials were evaporated at different rates and Compound HI-1 was deposited in a doping amount of 5 wt % based on the total amount of compounds HI-1 and HT-1 to form a hole injection layer having a thickness of 5 nm. Next, Compound HT-1 was deposited as a first hole transport layer having a thickness of 80 nm on the hole injection layer. Compound HT-2 was then introduced into another cell of the vacuum vapor deposition apparatus and was evaporated by applying an electric current to the cell, thereby forming a second hole transport layer having a thickness of 15 nm on the first hole transport layer. After forming the hole injection layer and the hole transport layers, a light-emitting layer was formed thereon as follows: Compound H-33 was introduced into a cell of the vacuum vapor deposition apparatus as a host, and Compound C-286 was introduced into another cell as a dopant. The two materials were evaporated at a different rate, and the dopant was deposited in a doping amount of 2 wt % based on the total amount of the host and dopant to form a light-emitting layer having a thickness of 22.5 nm on the second hole transport layer. Next, the hole blocking layer material described in the following Table 4 was deposited to a thickness of 5 nm on the light-emitting layer, and Compounds ET-2 and EI-1 as electron transport materials were deposited at a weight ratio of 2:1 to form an electron transport layer having a thickness of 25 nm. After depositing Liq as an electron injection layer having a thickness of 1 nm on the electron transport layer, an Al cathode having a thickness of 80 nm was deposited on the electron injection layer by another vacuum vapor deposition apparatus. Thus, an OLED was produced. Each compound used for all the materials were purified by vacuum sublimation under 10⁻⁶ torr.

### [Device Comparative Examples 3 and 4] Preparation of OLEDs Comprising a Conventional Compound as a Hole Blocking Layer Material

An OLED was manufactured in the same manner as Device Example 5 above, except that the compound shown in Table 4 below was used as a hole blocking layer material.

The time taken for luminance to decrease from 100% to 95% (lifespan: T95) at a luminance of 1,000 nits of the OLEDs of Device Example 5 and Device Comparative Examples 3 and 4 produced as described above, and the converted lifespan measure in terms of 100% conversion of the lifespan of Device Comparative Example 3, were measured, and the results are shown in Table 4 below.

TABLE 4

| | | | T95 Converted |
|---|---|---|---|
| | Material for Hole | Lifespan | Lifespan |
| | Blocking Layer | [T95, hr] | [%] |
| Device Example | N-1 | 44.0 | 110.3 |
| 5 | | | |
| Device | T-1 | 39.9 | 100.0 |
| Comparative | | | |
| Example 3 | | | |
| Device | T-2 | 40.8 | 102.3 |
| Comparative | | | |
| Example 4 | | | |

From Table 4, it can be seen that the organic electroluminescent device containing the compound according to the present disclosure in the hole blocking layer exhibits improved lifespan characteristics compared to the conventional organic electroluminescent device.

### [Device Examples 6 to 9] Preparing OLEDs Deposited with a Compound According to the Present Disclosure as an N-Type Charge Generation Layer

OLEDs according to the present disclosure were produced. First, a transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and thereafter was stored in isopropyl alcohol and then used. Thereafter, the ITO substrate was mounted on a substrate holder of a vacuum vapor deposition apparatus. Then, Compound HI-1 was introduced into a cell of the vacuum vapor deposition apparatus, and Compound HT-3 was introduced into another cell. The two materials were evaporated at different rates and Compound HI-1 was deposited in a doping amount of 3 wt % based on the total amount of compounds HI-1 and HT-3 to form a hole injection layer having a thickness of 5 nm. Next, Compound HT-3 was deposited as a first hole transport layer having a thickness of 30 nm on the hole injection layer. Next, Compound HT-4 was introduced into another cell of the vacuum vapor deposition apparatus and was evaporated by applying an electric current to the cell, thereby forming a second hole transport layer having a thickness of 5 nm on the first hole transport layer. After forming the hole injection layer and the hole transport layers, a first light-emitting layer was formed thereon as follows: Compound H-33 was introduced into two cells of the vacuum vapor deposition apparatus as a host, and Compound C-286 was introduced into another cell as a dopant. The two materials were evaporated at a different rate, and the dopant was deposited in a doping amount of 2 wt % based on the total amount of the host and dopant to form a first light-emitting layer having a thickness of 20 nm on the second hole transport layer. Next, a 5 nm thick hole blocking layer using compound ET-1 as a first hole blocking layer material was deposited on the first light-emitting layer. Next, Compound ET-3 as electron transport materials was deposited to form a first electron transport layer having a thickness of 10 nm. Thereafter, an N-type charge generation layer of 4 nm was formed by depositing 0.5 wt % of Li on the compound shown in Table 5 below. Next, Compound HI-1 was doped in an amount of 6 wt % based on the total amount of Compounds HI-1 and HT-3, and a P-type charge generation layer with a thickness of 10 nm was deposited. Next, Compound HT-3 was deposited to form a third hole transport layer having a thickness of 30 nm, and then Compound HT-4 was deposited to form a fourth hole transport layer having a thickness of 5 nm. A second light-emitting layer was formed thereon as follows: Compound H-33 was introduced into cell of the vacuum vapor deposition apparatus as a host, and Compound C-286 was introduced into another cell as a dopant. The two materials were evaporated at a different rate, and the dopant was deposited in a doping amount of 2 wt % based on the total amount of the host and dopant to form a second light-emitting layer having a thickness of 20 nm on the fourth hole transport layer. Next, a 5 nm thick hole blocking layer using Compound ET-1 as a second hole blocking layer material was deposited on the second light-emitting layer. Compounds ET-2 and EI-1 were respectively added to two cells in the vacuum vapor deposition apparatus as a second electron transport layer material, and then the two materials were deposited to a thickness of 25 nm at a weight ratio of 2:1. Next, Yb was deposited as an electron injection layer to a thickness of 1 nm on the second electron transport layer, and then an Al cathode was deposited to a thickness of 80 nm on the electron injection layer using another vacuum vapor deposition apparatus. Thus, OLEDs were produced. Each compound used for all the materials were purified by vacuum sublimation under 10⁻⁸ torr.

### [Device Comparative Example 5] Preparing an OLED Deposited with a Conventional Compound as an N-Type Charge Generation Layer

An OLED was manufactured in the same manner as Device Example 6 above, except that the compound shown in Table 5 below was used as the N-type charge generation layer material.

The time taken for luminance to decrease from 100% to 95% (lifespan: T95) at a luminance of 1,000 nits of the OLEDs of Device Examples 6 to 9 and Device Comparative Example 5 produced as described above, was measured, and the results are shown in Table 5 below.

TABLE 5

| | N-TYPE CHARGE | Lifespan |
|---|---|---|
| | GENERATION LAYER | T95 [hr] |
| Device Example 6 | N-176 | 159.4 |
| Device Example 7 | N-216 | 162.5 |
| Device Example 8 | N-3 | 164.8 |
| Device Example 9 | N-251 | 163.6 |
| Device Comparative | T-3 | 141.7 |
| Example 5 | | |

### [Device Examples 10 and 11] Preparing OLEDs Deposited with a Compound According to the Present Disclosure as an N-Type Charge Generation Layer

OLEDs were manufactured in the same manner as Device Example 6 above, except that Compound ET-2 was deposited as a first electron transport layer material with a thickness of 15 nm, and each of the compounds in Table 6 below was used as the N-type charge generation layer material.

### [Device Comparative Example 6] Preparing an OLED Deposited with a Conventional Compound as an N-Type Charge Generation Layer

An OLED was manufactured in the same manner as Device Example 9 above, except that the compound shown in Table 6 below was used as the N-type charge generation layer material.

The current efficiency and the time taken for luminance to decrease from 100% to 95% (lifespan: T95) at a luminance of 1,000 nits of the OLEDs of Device Examples 10 and 11 and Device Comparative Example 6 produced as described above, were measured, and the results thereof are shown in the following Table 6.

TABLE 6

| | | Current | |
|---|---|---|---|
| | N-Type Charge | Efficiency | Lifespan |
| | Generation Layer | [cd/A] | T95 [hr] |
| Device Example 10 | N-3 | 8.4 | 166.8 |
| Device Example 11 | N-16 | 8.3 | 154.6 |
| Device Comparative | T-3 | 8.2 | 134.1 |
| Example 6 | | | |

From Tables 5 and 6, it can be seen that the organic electroluminescent device containing the organic electroluminescent compound according to the present disclosure in the N-type charge generation layer exhibits improved current efficiency and/or significantly improved lifespan characteristics.

The compounds used in Device Examples 5 to 11 and Device Comparative Examples 3 to 6 above are shown in the following Table 7.

TABLE 7

| | |
|---|---|
| Hole Injection Layer/Hole Transport Layer | 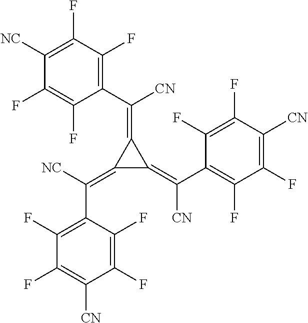 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F] |
| | HI-1 |
| | 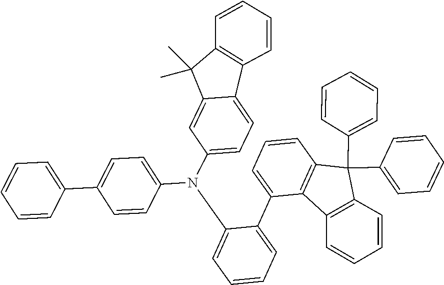 [SMILES: CC1(C)c2ccccc2-c2ccc(N(c3ccc(-c4ccccc4)cc3)c3ccccc3-c3cccc4c3-c3ccccc3C4(c3ccccc3)c3ccccc3)cc21] |
| | HT-1 |
| | 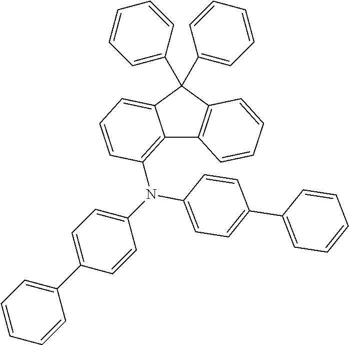 [SMILES: c1ccc(-c2ccc(N(c3ccc(-c4ccccc4)cc3)c3cccc4c3-c3ccccc3C4(c3ccccc3)c3ccccc3)cc2)cc1] |
| | HT-2 |
| | 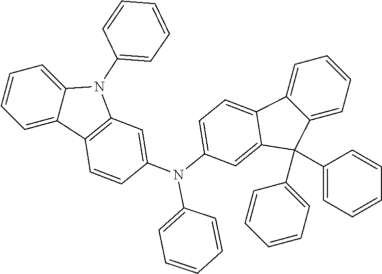 [SMILES: c1ccc(N(c2ccc3c(c2)C(c2ccccc2)(c2ccccc2)c2ccccc2-3)c2ccc3c4ccccc4n(-c4ccccc4)c3c2)cc1] |
| | HT-3 |
| | 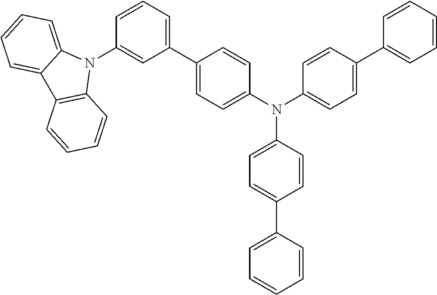 [SMILES: c1ccc(-c2ccc(N(c3ccc(-c4ccccc4)cc3)c3ccc(-c4cccc(-n5c6ccccc6c6ccccc65)c4)cc3)cc2)cc1] |
| | HT-4 |
| Light-Emitting Layer | 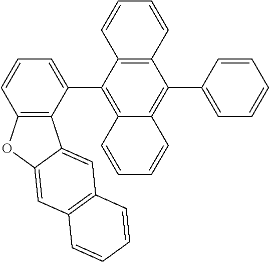 [SMILES: c1ccc(-c2c3ccccc3c(-c3cccc4oc5cc6ccccc6cc5c34)c3ccccc23)cc1] |
| | H-33 |
| | 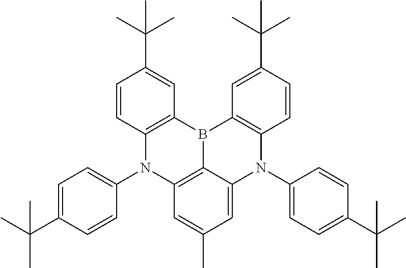 [SMILES: Cc1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1] |
| | C-286 |
| Electron Transport Layer/ Electron Injection Layer | 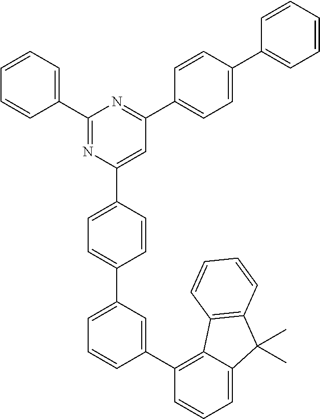 [SMILES: CC1(C)c2ccccc2-c2c(-c3cccc(-c4ccc(-c5cc(-c6ccc(-c7ccccc7)cc6)nc(-c6ccccc6)n5)cc4)c3)cccc21] |
| | ET-1 |
| | 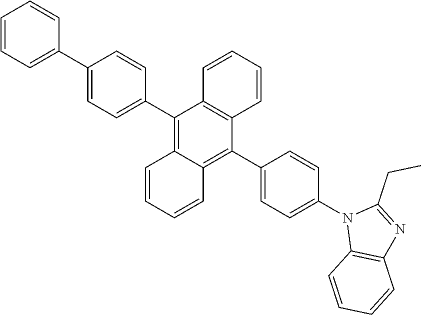 [SMILES: CCc1nc2ccccc2n1-c1ccc(-c2c3ccccc3c(-c3ccc(-c4ccccc4)cc3)c3ccccc23)cc1] |
| | ET-2 |
| | 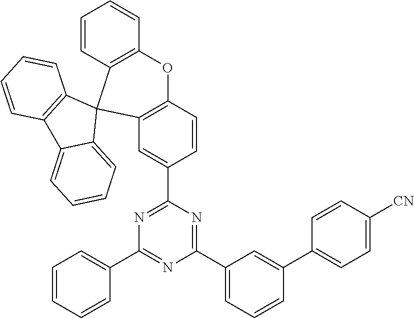 [SMILES: N#Cc1ccc(-c2cccc(-c3nc(-c4ccccc4)nc(-c4ccc5c(c4)C4(c6ccccc6O5)c5ccccc5-c5ccccc54)n3)c2)cc1] |
| | ET-3 |
| | 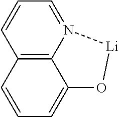 |
| | EI-1 |
| Hole Blocking Layer/ N-type Charge Generation Layer | 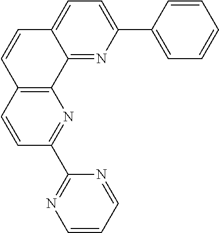 [SMILES: c1ccc(-c2ccc3ccc4ccc(-c5ncccn5)nc4c3n2)cc1] |
| | N-1 |
| | 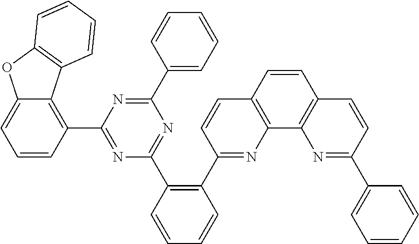 [SMILES: c1ccc(-c2ccc3ccc4ccc(-c5ccccc5-c5nc(-c6ccccc6)nc(-c6cccc7oc8ccccc8c67)n5)nc4c3n2)cc1] |
| | N-16 |
| | 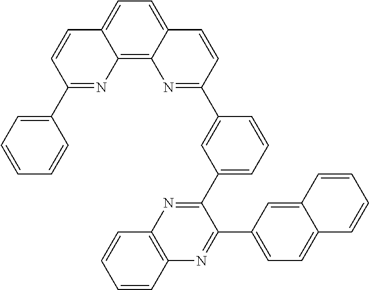 [SMILES: c1ccc(-c2ccc3ccc4ccc(-c5cccc(-c6nc7ccccc7nc6-c6ccc7ccccc7c6)c5)nc4c3n2)cc1] |
| | N-251 |
| | 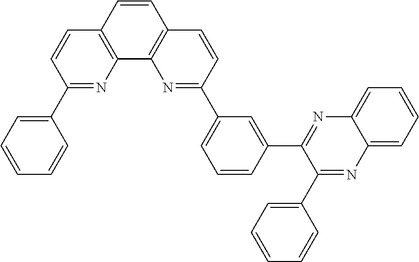 [SMILES: c1ccc(-c2ccc3ccc4ccc(-c5cccc(-c6nc7ccccc7nc6-c6ccccc6)c5)nc4c3n2)cc1] |
| | N-176 |
| | 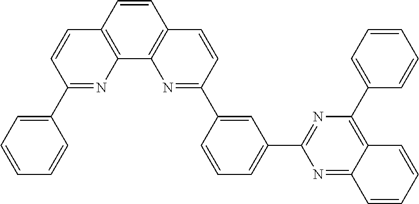 [SMILES: c1ccc(-c2ccc3ccc4ccc(-c5cccc(-c6nc(-c7ccccc7)c7ccccc7n6)c5)nc4c3n2)cc1] |
| | N-216 |
| | 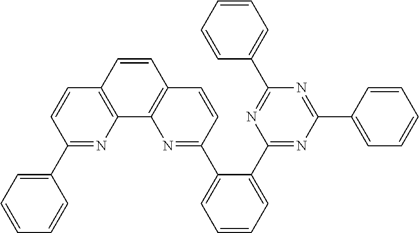 [SMILES: c1ccc(-c2ccc3ccc4ccc(-c5ccccc5-c5nc(-c6ccccc6)nc(-c6ccccc6)n5)nc4c3n2)cc1] |
| | N-3 |
| | 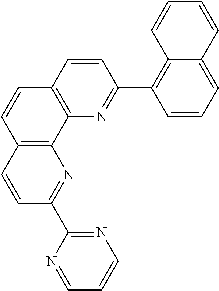 [SMILES: c1cnc(-c2ccc3ccc4ccc(-c5cccc6ccccc56)nc4c3n2)nc1] |
| | T-1 |
| | 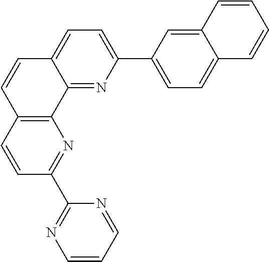 [SMILES: c1cnc(-c2ccc3ccc4ccc(-c5ccc6ccccc6c5)nc4c3n2)nc1] |
| | T-2 |
| | 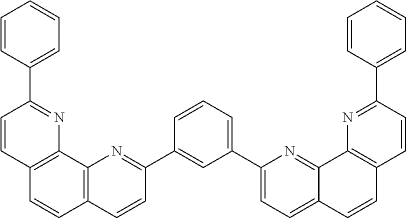 [SMILES: c1ccc(-c2ccc3ccc4ccc(-c5cccc(-c6ccc7ccc8ccc(-c9ccccc9)nc8c7n6)c5)nc4c3n2)cc1] |
| | T-3 |

### [Device Example 12] Preparing an OLED Deposited with Compounds According to the Present Disclosure as Materials for an N-Type Charge Generation Layer and a Light-Emitting Layer

An OLED according to the present disclosure was produced. First, a transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and thereafter was stored in isopropyl alcohol and then used. Thereafter, the ITO substrate was mounted on a substrate holder of a vacuum vapor deposition apparatus. Then, Compound HI-1 was introduced into a cell of the vacuum vapor deposition apparatus, and Compound HT-3 was introduced into another cell. The two materials were evaporated at different rates and Compound HI-1 was deposited in a doping amount of 3 wt % based on the total amount of compounds HI-1 and HT-3 to form a hole injection layer having a thickness of 5 nm. Next, Compound HT-3 was deposited as a first hole transport layer having a thickness of 30 nm on the hole injection layer. Next, Compound HT-5 was introduced into another cell of the vacuum vapor deposition apparatus and was evaporated by applying an electric current to the cell, thereby forming a second hole transport layer having a thickness of 25 nm on the first hole transport layer. After forming the hole injection layer and the hole transport layers, a first light-emitting layer was formed thereon as follows: A 1:1 mixture of compounds H4-121-D16 and H5-1-D11 was added as hosts to two cells in the vacuum vapor deposition apparatus, and Compound RD-1 was added as a dopant to another cell. Then, the two host materials were evaporated and the dopant material was evaporated at a different rate, simultaneously, and was deposited in a doping amount of 3 wt % based on the total amount of the hosts and dopant to form a first light-emitting layer having a thickness of 40 nm on the second hole transport layer. Next, a 5 nm thick hole blocking layer using Compound ET-1 as a first hole blocking layer material was deposited on the first light-emitting layer. Next, Compound ET-3 as electron transport materials was deposited to form a first electron transport layer having a thickness of 10 nm. Thereafter, an N-type charge generation layer of 4 nm was formed by depositing 0.5 wt % of Li on the compound shown in Table 8 below. Next, Compound HI-1 was doped in an amount of 6 wt % based on the total amount of Compounds HI-1 and HT-3, and a P-type charge generation layer with a thickness of 10 nm was deposited. Next, Compound HT-3 was deposited to form a third hole transport layer having a thickness of 30 nm, and then Compound HT-5 was deposited to form a fourth hole transport layer having a thickness of 25 nm. A second light-emitting layer was formed thereon as follows: A 1:1 mixture of Compounds H4-121-D16 and H5-1-D11 was added as hosts to two cells in the vacuum vapor deposition apparatus, and Compound RD-1 was added as a dopant to another cell. Then, the two host materials were evaporated at a rate of 1:1 and the dopant material was evaporated at a different rate, simultaneously, and the dopant was deposited in a doping amount of 3 wt % based on the total amount of the hosts and dopant to form a second light-emitting layer having a thickness of 40 nm on the fourth hole transport layer. Next, a 5 nm thick hole blocking layer using compound ET-1 as a second hole blocking layer material was deposited on the second light-emitting layer. Compounds ET-2 and EI-1 were respectively added to two cells in the vacuum vapor deposition apparatus as a second electron transport layer material, and then the two materials were deposited to a thickness of 25 nm at a weight ratio of 2:1. Next, Yb was deposited as an electron injection layer to a thickness of 1 nm on the second electron transport layer, and then an Al cathode was deposited to a thickness of 80 nm on the electron injection layer using another vacuum vapor deposition apparatus. Thus, an OLED was produced. Each compound used for all the materials were purified by vacuum sublimation under 10$ torr.

### [Device Comparative Example 7] Preparing an OLED Deposited with a Non-Deuterated Compound as Materials for the N-Type Charge Generation Layer and Light-Emitting Layer

An OLED was manufactured in the same manner as Device Example 12 above, except that each of the compounds in Table 8 below was used as the host material of the light-emitting layer and the N-type charge generation layer material.

The time taken for luminance to decrease from 100% to 95% (lifespan: T95) at a luminance of 1,000 nits according to the OLEDs of Device Example 12 and Device Comparative Example 7 produced as described above, was measured, and the results thereof are shown in the following Table 8.

TABLE 8

| | | | Hosts in Second | |
|---|---|---|---|---|
| | Hosts in First Light- | N-Type Charge | Light-emitting | Lifespan |
| | emitting Layer | Generation Layer | Layer | T95 [hr] |
| Device Example | H4-121-D16:H5-1- | N-146-D10 | H4-121-D16:H5-1- | 502.6 |
| 12 | D11 | | D11 | |
| | (1:1) | | (1:1) | |
| Device | H4-246:H5-51 | N-74 | H4-246:H5-51 | 205.7 |
| Comparative | (1:1) | | (1:1) | |
| Example 7 | | | | |

The compounds used in Device Example 12 and Device Comparative Example 7 above are shown in the following Table 9.

TABLE 9

| | |
|---|---|
| Hole Injection Layer/Hole Transport Layer | 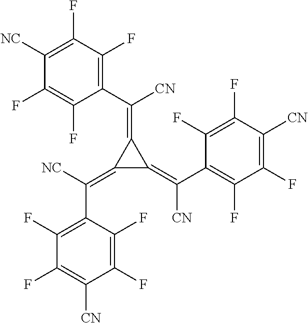 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F] |
| | HI-1 |
| | 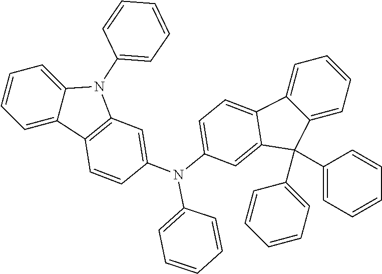 [SMILES: c1ccc(N(c2ccc3c(c2)C(c2ccccc2)(c2ccccc2)c2ccccc2-3)c2ccc3c4ccccc4n(-c4ccccc4)c3c2)cc1] |
| | HT-3 |
| | 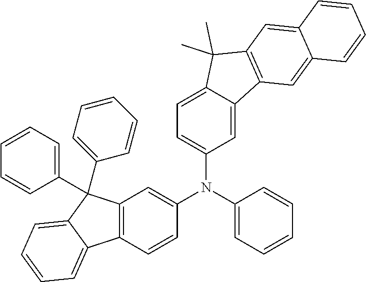 [SMILES: CC1(C)c2ccc(N(c3ccccc3)c3ccc4c(c3)C(c3ccccc3)(c3ccccc3)c3ccccc3-4)cc2-c2cc3ccccc3cc21] |
| | HT-5 |
| Light-Emitting Layer | 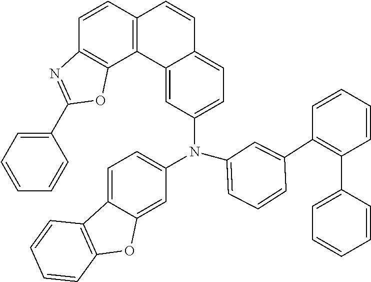 [SMILES: c1ccc(-c2nc3ccc4ccc5ccc(N(c6cccc(-c7ccccc7-c7ccccc7)c6)c6ccc7c(c6)oc6ccccc67)cc5c4c3o2)cc1] |
| | H4-246 |
| | 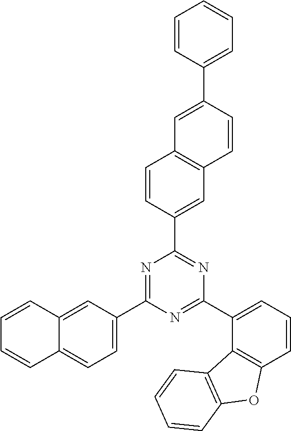 [SMILES: c1ccc(-c2ccc3cc(-c4nc(-c5ccc6ccccc6c5)nc(-c5cccc6oc7ccccc7c56)n4)ccc3c2)cc1] |
| | H5-51 |
| | 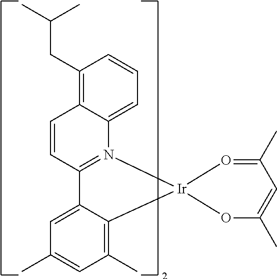 |
| | RD-1 |
| | 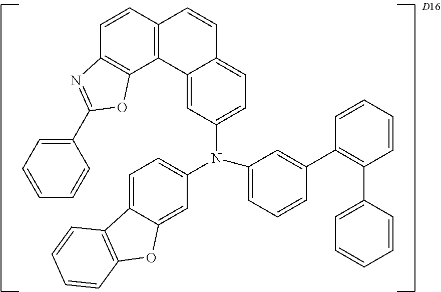 |
| | H4-121-D16 |
| | 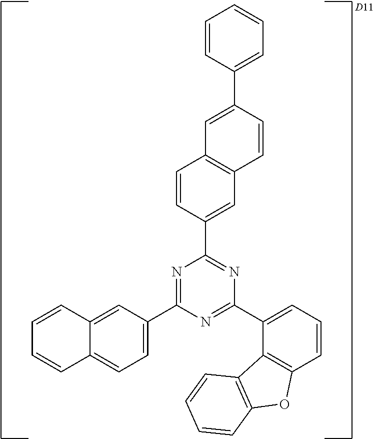 |
| | H5-1-D11 |
| Electron Transport Layer/Hole Blocking Layer/ Electron Injection Layer | 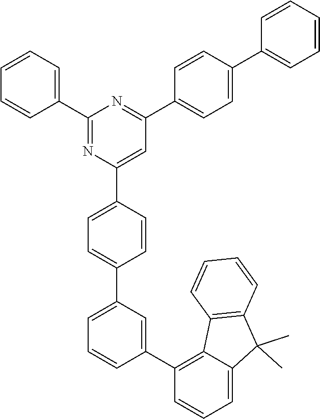 [SMILES: CC1(C)c2ccccc2-c2c(-c3cccc(-c4ccc(-c5cc(-c6ccc(-c7ccccc7)cc6)nc(-c6ccccc6)n5)cc4)c3)cccc21] |
| | ET-1 |
| | 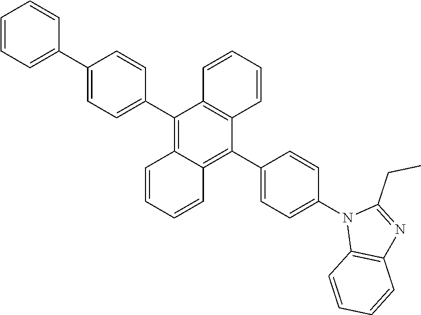 [SMILES: CCc1nc2ccccc2n1-c1ccc(-c2c3ccccc3c(-c3ccc(-c4ccccc4)cc3)c3ccccc23)cc1] |
| | ET-2 |
| | 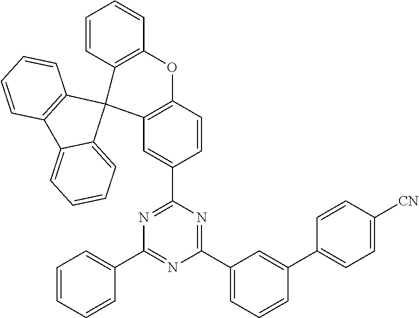 [SMILES: N#Cc1ccc(-c2cccc(-c3nc(-c4ccccc4)nc(-c4ccc5c(c4)C4(c6ccccc6O5)c5ccccc5-c5ccccc54)n3)c2)cc1] |
| | ET-3 |
| | 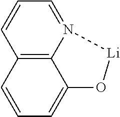 |
| | EI-1 |
| N-type Charge Generation Layer | 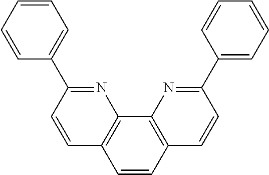 [SMILES: c1ccc(-c2ccc3ccc4ccc(-c5ccccc5)nc4c3n2)cc1] |
| | N-74 |
| | 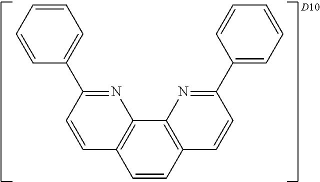 |
| | N-164-D10 |

### [Device Example 13] Preparing an OILED Deposited with Compounds According to the Present Disclosure as Materials for an N-Type Charge Generation Layer and a Light-Emitting Layer

An OLED according to the present disclosure was produced. First, a transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and thereafter was stored in isopropyl alcohol and then used. Thereafter, the ITO substrate was mounted on a substrate holder of a vacuum vapor deposition apparatus. Then, Compound HI-1 was introduced into a cell of the vacuum vapor deposition apparatus, and Compound HT-3 was introduced into another cell. The two materials were evaporated at different rates and Compound HI-1 was deposited in a doping amount of 3 wt % based on the total amount of compounds HI-1 and HT-3 to form a hole injection layer having a thickness of 5 nm. Next, Compound HT-3 was deposited as a first hole transport layer having a thickness of 30 nm on the hole injection layer. Next, Compound HT-6 was introduced into another cell of the vacuum vapor deposition apparatus and was evaporated by applying an electric current to the cell, thereby forming a second hole transport layer having a thickness of 15 nm on the first hole transport layer. After forming the hole injection layer and the hole transport layers, a first light-emitting layer was formed thereon as follows: A 2:1 mixture of compounds H2-83-D23 and H6-22-D8 was added as hosts to two cells in the vacuum vapor deposition apparatus, and Compound GD-1 was added as a dopant to another cell. Then, the two host materials were evaporated at a rate of 2:1 and the dopant material was evaporated at a different rate, simultaneously. The dopant was deposited in a doping amount of 10 wt % based on the total amount of the hosts and dopant to form a first light-emitting layer having a thickness of 36 nm on the second hole transport layer. Next, a 5 nm thick hole blocking layer using Compound ET-1 as a first hole blocking layer material was deposited on the first light-emitting layer. Next, Compound ET-3 as electron transport materials was deposited to form a first electron transport layer having a thickness of 10 nm. Thereafter, an N-type charge generation layer of 4 nm was formed by depositing 0.5 wt % of Li on the compound shown in Table 10 below. Next, Compound HI-1 was doped in an amount of 6 wt % based on the total amount of compounds HI-1 and HT-3, and a P-type charge generation layer with a thickness of 10 nm was deposited. Next, Compound HT-3 was deposited to form a third hole transport layer having a thickness of 30 nm, and then Compound HT-6 was deposited to form a fourth hole transport layer having a thickness of 15 nm. A second light-emitting layer was formed thereon as follows: A 2:1 mixture of compounds H2-83-D23 and H6-22-D8 was added as hosts to two cells in the vacuum vapor deposition apparatus, and Compound GD-1 was added as a dopant to another cell. Then, the two host materials were evaporated at a rate of 2:1 and the dopant material was evaporated at a different rate, simultaneously. The dopant was deposited in a doping amount of 10 wt % based on the total amount of the hosts and dopant to form a second light-emitting layer having a thickness of 36 nm on the fourth hole transport layer. Next, a 5 nm thick hole blocking layer using Compound ET-1 as a second hole blocking layer material was deposited on the second light-emitting layer. Compounds ET-2 and EI-1 were respectively added to two cells in the vacuum vapor deposition apparatus as a second electron transport layer material, and then the two materials were deposited to a thickness of 25 nm at a weight ratio of 2:1. Next, Yb was deposited as an electron injection layer to a thickness of 1 nm on the second electron transport layer, and then an Al cathode was deposited to a thickness of 80 nm on the electron injection layer using another vacuum vapor deposition apparatus. Thus, an OLED was produced. Each compound used for all the materials were purified by vacuum sublimation under 10⁻⁸ torr.

### [Device Comparative Example 8] Preparing an OLED Deposited with a Non-Deuterated Compound as Materials for the N-Type Charge Generation Layer and Light-Emitting Layer

An OLED was manufactured in the same manner as Device Example 13 above, except that each of the compounds in Table 10 below was used as the host material of the light-emitting layer and the N-type charge generation layer material

The time taken for luminance to decrease from 100% to 95% (lifespan: T95) at a luminance of 40,000 nits according to the OLEDs of Device Example 13 and Device Comparative Example 8 produced as described above, was measured, and the results thereof are shown in the following Table 10.

TABLE 10

| | | | Hosts in Second | |
|---|---|---|---|---|
| | Hosts in First Light- | N-Type Charge | Light-emitting | Lifespan |
| | emitting Layer | Generation Layer | Layer | T95 [hr] |
| Device Example | H2-83-D23:H6-22-D8 | N-146-D10 | H2-83-D23:H6-22- | 97.3 |
| 13 | (2:1) | | D8 | |
| | | | (2:1) | |
| Device | H2-33:H6-23 | N-74 | H2-33:H6-23 | 90.4 |
| Comparative | (2:1) | | (2:1) | |
| Example 8 | | | | |

From Tables 8 and 10, it can be seen that the organic electroluminescent device containing the deuterated compound according to the present invention in the plurality of host materials of the light emitting layer and the N-type charge generation layer exhibits significantly improved lifespan characteristics.

The compounds used in Device Example 13 and Device Comparative Example 8 above are shown in the following Table 11.

TABLE 11

| | |
|---|---|
| Hole Injection Layer/Hole Transport Layer | 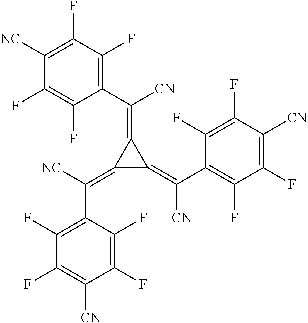 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F] |
| | HI-1 |
| | 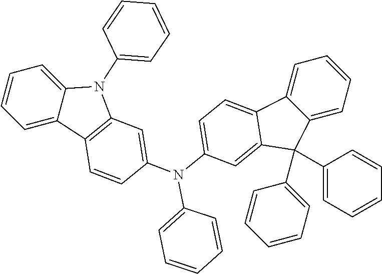 [SMILES: c1ccc(N(c2ccc3c(c2)C(c2ccccc2)(c2ccccc2)c2ccccc2-3)c2ccc3c4ccccc4n(-c4ccccc4)c3c2)cc1] |
| | HT-3 |
| | 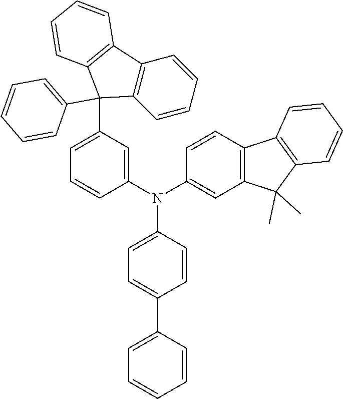 [SMILES: CC1(C)c2ccccc2-c2ccc(N(c3ccc(-c4ccccc4)cc3)c3cccc(C4(c5ccccc5)c5ccccc5-c5ccccc54)c3)cc21] |
| | HT-6 |
| Light-Emitting Layer | 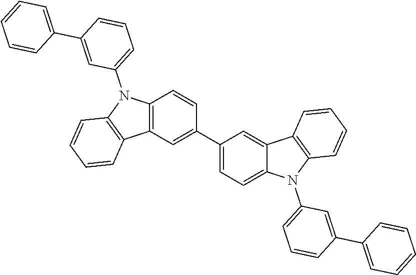 [SMILES: c1ccc(-c2cccc(-n3c4ccccc4c4cc(-c5ccc6c(c5)c5ccccc5n6-c5cccc(-c6ccccc6)c5)ccc43)c2)cc1] |
| | H2-33 |
| | 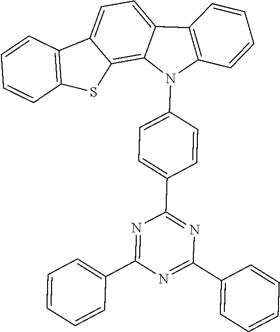 [SMILES: c1ccc(-c2nc(-c3ccccc3)nc(-c3ccc(-n4c5ccccc5c5ccc6c7ccccc7sc6c54)cc3)n2)cc1] |
| | H6-23 |
| | 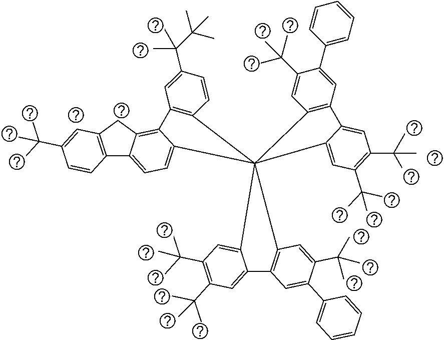 |
| | GD-1 |
| | 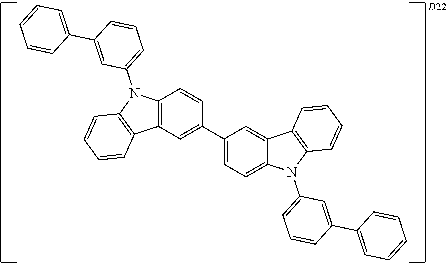 |
| | H2-83-D23 |
| | 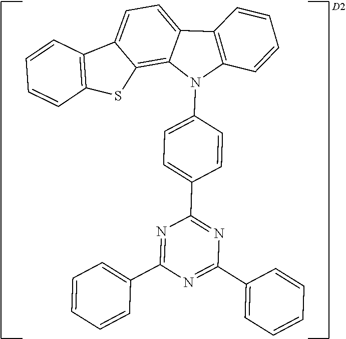 |
| | H6-22-D8 |
| Electron Transport Layer/Hole Blocking Layer/ Electron Injection Layer | 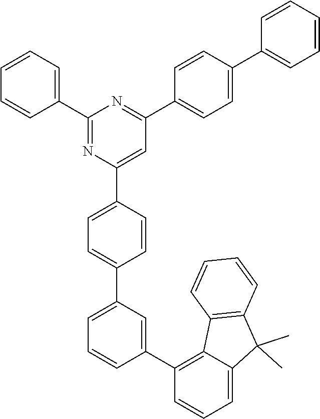 [SMILES: CC1(C)c2ccccc2-c2c(-c3cccc(-c4ccc(-c5cc(-c6ccc(-c7ccccc7)cc6)nc(-c6ccccc6)n5)cc4)c3)cccc21] |
| | ET-1 |
| | 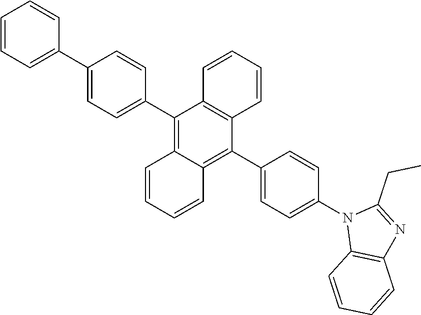 [SMILES: CCc1nc2ccccc2n1-c1ccc(-c2c3ccccc3c(-c3ccc(-c4ccccc4)cc3)c3ccccc23)cc1] |
| | ET-2 |
| | 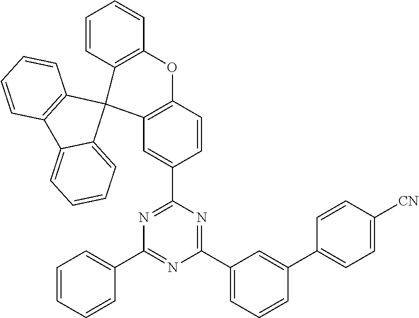 [SMILES: N#Cc1ccc(-c2cccc(-c3nc(-c4ccccc4)nc(-c4ccc5c(c4)C4(c6ccccc6O5)c5ccccc5-c5ccccc54)n3)c2)cc1] |
| | ET-3 |
| | 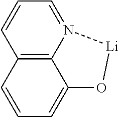 |
| | EI-1 |
| N-type Charge Generation Layer | 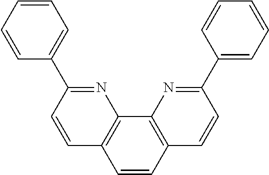 [SMILES: c1ccc(-c2ccc3ccc4ccc(-c5ccccc5)nc4c3n2)cc1] |
| | N-74 |
| | 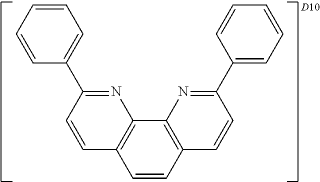 |
| | N-164-D10 |

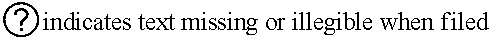

### SYMBOL DESCRIPTION

| | |
|---|---|
| 10, 20: Organic Electroluminescent Device | |
| 110: First Electrode | 410: Second Electrode |
| 200, 300: Light-emitting Unit | 500: Charge Generation Layer |
| 210: Hole Injection Layer | |
| 220, 230, 320, 330: Hole Transport Layer | |
| 240, 340: Light-emitting Layers | 250, 350: Hole Blocking Layer |
| 260, 360: Electron Transport Layer | 370: Electron Injection Layer |
| 510: N-type Charge Generation Layer | |
| 520: P-type Charge Generation Layer | |

1. An organic electroluminescent device comprises a plurality of light-emitting units positioned between a first electrode and a second electrode; and at least one charge generation layer positioned between the adjacent light-emitting units, wherein the light-emitting units comprise at least one light-emitting layer, and at least one of the light-emitting layer and the charge generation layer comprises a deuterated compound.

2. The organic electroluminescent device of claim 1, wherein the deuterium substitution rate of the deuterated compound is 30% to 100%.

3. The organic electroluminescent device of claim 1, wherein the deuterium substitution rate of the deuterated compound is 50% to 100%.

4. The organic electroluminescent device of claim 1, wherein the light-emitting layer includes a compound represented by the following Formula 1-1. 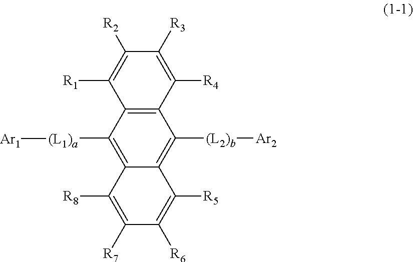
wherein,
L₁ and L₂ each independently represent, a single bond, a substituted or unsubstituted (C6-C30)arylene, or a substituted or unsubstituted (3- to 30-membered)heteroarylene;
Ar₁ and Ar₂ each independently represent, a substituted or unsubstituted (C6-C30)aryl or a substituted or unsubstituted (3- to 30-membered)heteroaryl;
R₁ to R₆ each independently represent, hydrogen, deuterium, halogen, cyano, a substituted or unsubstituted (C1-C30)alkyl, a substituted or unsubstituted (C2-C30)alkenyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring of (C3-C30) aliphatic ring and an (C6-C30) aromatic ring, a substituted or unsubstituted mono- or di-(C1-C30)alkylamino, a substituted or unsubstituted mono- or di-(C2-C30)alkenylamino, a substituted or unsubstituted mono- or di-(C6-C30)arylamino, a substituted or unsubstituted mono- or di-(3- to 30-membered)heteroarylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino;
a and b each independently represent, an integer of 1 or 2;
when a and b are 2 or more, each of L₁ and L₂ may be the same or different.

5. The organic electroluminescent device of claim 1, wherein the light-emitting layer includes two or more of compounds represented by the following formulas 1-2 to 1-4. 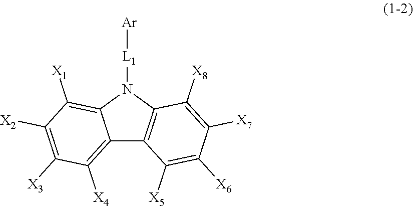
wherein,
Ar represents a substituted or unsubstituted (C6-C30)aryl or a substituted or unsubstituted (3- to 30-membered)heteroaryl;
L₁ represents a single bond, a substituted or unsubstituted (C6-C30)arylene, or a substituted or unsubstituted (3- to 30-membered)heteroarylene;
X₁ to X₈ each independently represent, hydrogen, deuterium, halogen, cyano, carboxyl, nitro, hydroxy, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, a substituted or unsubstituted (3- to 30-membered)heteroaryl, —NX₁₁X₁₂ or —SiX₁₃X₁₄X₁₅; or the adjacent at least two of X₁ to X₆ may be linked to each other to form a ring(s);
X₁₁ to X₁₅ each independently represent, hydrogen, deuterium, halogen, cyano, carboxyl, nitro, hydroxy, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, or a substituted or unsubstituted (3- to 30-membered)heteroaryl; or may be linked to the adjacent substituents to form a ring(s):
HAr-(L₁-Ar₂)_{d} (1-3)
wherein,
HAr represents a substituted or unsubstituted, nitrogen-containing (3- to 20-membered)heteroaryl;
L₂ represents a single bond or a substituted or unsubstituted (C6-C30)arylene;
Ar₂ represents a substituted or unsubstituted (C6-C30)aryl or a substituted or unsubstituted (C3-C30)heteroaryl; and
d is an integer of 1 to 3, when d is 2 or more, each of (L₂-Ar₂) may be the same or different; 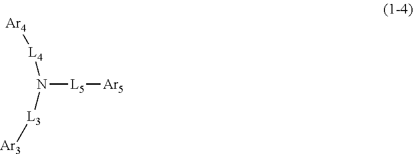
wherein,
Ar₃ to Ar₅ each independently represent, hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or —N(Ar₁₁)(Ar₁₂);
Ar₁₁ and Ar₁₂ each independently represent, a substituted or unsubstituted (C1-C30)alkyl, a substituted or unsubstituted (C2-C30)alkenyl, a substituted or unsubstituted (C6-C30)aryl, a substituted or unsubstituted (3- to 30-membered)heteroaryl;
L₃ to L₅ each independently represent, a single bond, a substituted or unsubstituted (C1-C30)alkylene, a substituted or unsubstituted (C6-C30)arylene, a substituted or unsubstituted (3- to 30-membered)heteroarylene, or a substituted or unsubstituted (C3-C30)cycloalkylene;
provided that the cases where all of L₃ to L₅ are a single bond, while all of Ar₃ to Ar₅ are hydrogen are excluded.

6. The organic electroluminescent device according to claim 5, wherein the compound represented by Formula 1-2 is represented by following Formula 1-2-1 or 1-2-2. 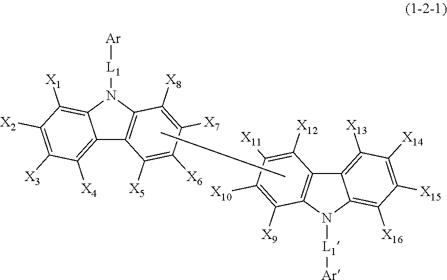
wherein,
X₁ to X₈ and L₁ are as defined in claim 5;
Ar and Ar′ each independently represent, a substituted or unsubstituted (C6-C30)aryl, a substituted or unsubstituted dibenzofuranyl, a substituted or unsubstituted dibenzothiophenyl, or a substituted or unsubstituted carbazolyl;
L₁′ is the same as the definition of L₁ in claim 5;
X₉ to X₁₈ are the same as the definition of X₁ to X₈ in claim 5; and
one of X₁ to X₈ and one of X₉ to X₁₆ are linked to each other to form a single bond; 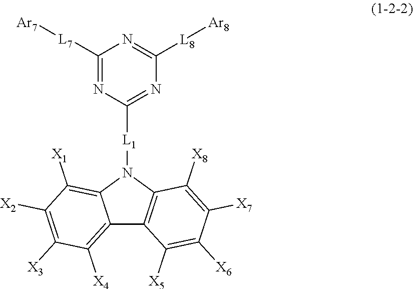
wherein,
X₁ to X₈ and L₁ are as defined in claim 5;
L₇ and L₈ are the same as the definition of L₁ in claim 5;
Ar₇ and Ar₈ each independently represent, a substituted or unsubstituted (C1-C30)alkyl, a substituted or unsubstituted (C6-C30)aryl or a substituted or unsubstituted (C3-C30)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino.

7. The organic electroluminescent device of claim 5, wherein Ar₂ of Formula 1-3 and at least one of Ar₃ to Ar₅ of Formula 1-4 are represented by any one of the following formulas 1-3-1 to 1-3-3. 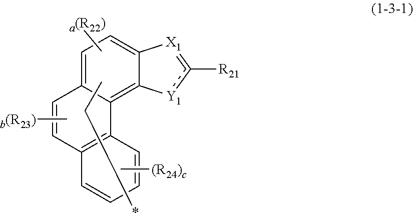
wherein,
X₁ and Y₁ each independently represent, —N═, —NR₂₅—, —O— or —S—; provided that, one of X₁ and Y₁ represents —N═, the other of X₁ and Y₁ represents —NR₂₅—, —O— or —S—;
R₂₁ represents a substituted or unsubstituted (C6-C30)aryl or a substituted or unsubstituted (3- to 30-membered)heteroaryl;
R₂₂ to R₂₅ each independently represent, hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or may be linked to an adjacent substituent to form a ring or may be linked to an adjacent substituent to form a ring:
a and b each independently represent, 1 or 2, c is an integer of 1 to 4, when a to c are 2 or more, each of R₂ to each of R₂₄ may be the same or different;
* represents a linking site with L₂ of Formula 1-3, or a linking site with L₈ to L₅ of Formula 1-4; 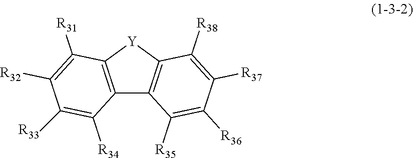
wherein,
Y represents —O—, —S, or —NR₃₉;
R₃₉ represents a substituted or unsubstituted (C6-C30)aryl;
R₃₁ to R₃₈ each independently represent, a linking site with L₂ of Formula 1-3, or a linking site with L₃ to L₅ of Formula 1-4; hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or may be linked to an adjacent substituent to form a ring; 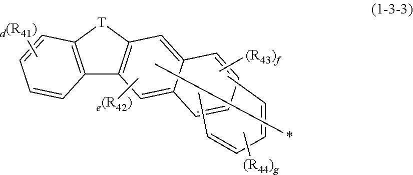
wherein,
T represents —O—, —S—, —CR₄₅R₄₆, —NR₄₇, or —Se—;
R₄₅ to R₄₇ each independently represent, hydrogen, deuterium, halogen, a substituted or unsubstituted (C1-C30)alkyl, a substituted or unsubstituted (C6-C30)aryl;
R₄₁ to R₄₄ each independently represent, hydrogen, deuterium, halogen, a substituted or unsubstituted (C1-C30)alkyl, a substituted or unsubstituted (C6-C30)aryl, a substituted or unsubstituted (3- to 30-membered)heteroaryl or —N(Ar₂₁)(Ar₂₂); or may be linked to an adjacent substituent to form a ring;
Ar₂₁ and Ar₂₂ each independently represent, a substituted or unsubstituted (C1-C30)alkyl, a substituted or unsubstituted (C2-C30)alkenyl, a substituted or unsubstituted (C6-C30)aryl, a substituted or unsubstituted (3- to 30-membered)heteroaryl;
d and g each independently represent, an integer of 1 to 4, e and f each independently represent, an integer of 1 or 2;
when d to g are 2 or more, each of R₄₁ to R₄₄ may be the same or different; and
* represent a linking site with L₂ of Formula 1-3, or a linking site with L₃ to L₅ of Formula 1-4.

8. The organic electroluminescent device of claim 1, wherein the charge generation layer includes a compound represented by the following Formula 2. 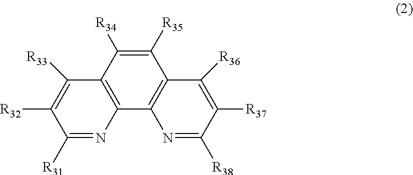
wherein,
R₃, to R₃₈ each independently represent, hydrogen, deuterium, halogen, cyano, a substituted or unsubstituted (C1-C30)alkyl, a substituted or unsubstituted (C2-C30)alkenyl, a substituted or unsubstituted (C6-C30)aryl, a substituted or unsubstituted (3- to 30-membered)heteroaryl, a substituted or unsubstituted (C3-C30)cycloalkyl, a substituted or 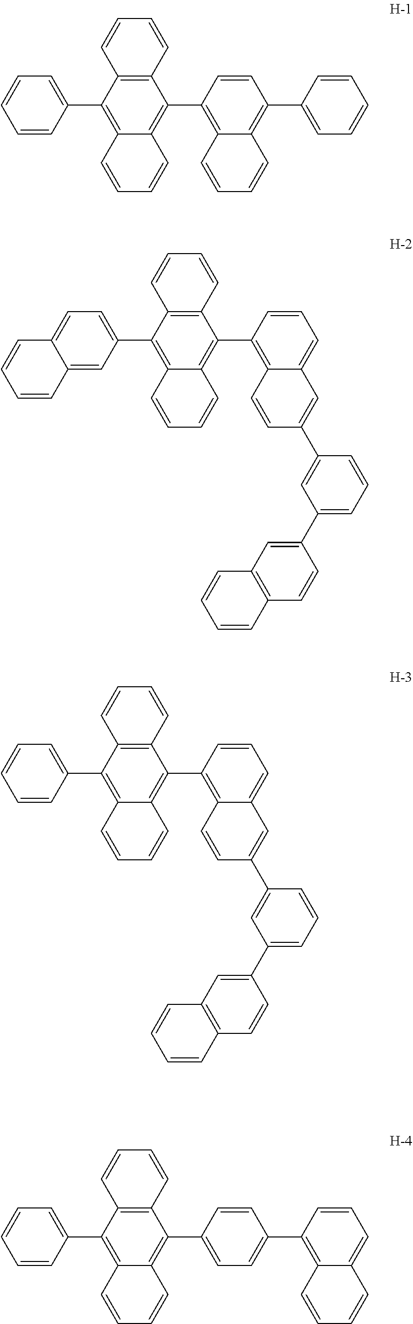 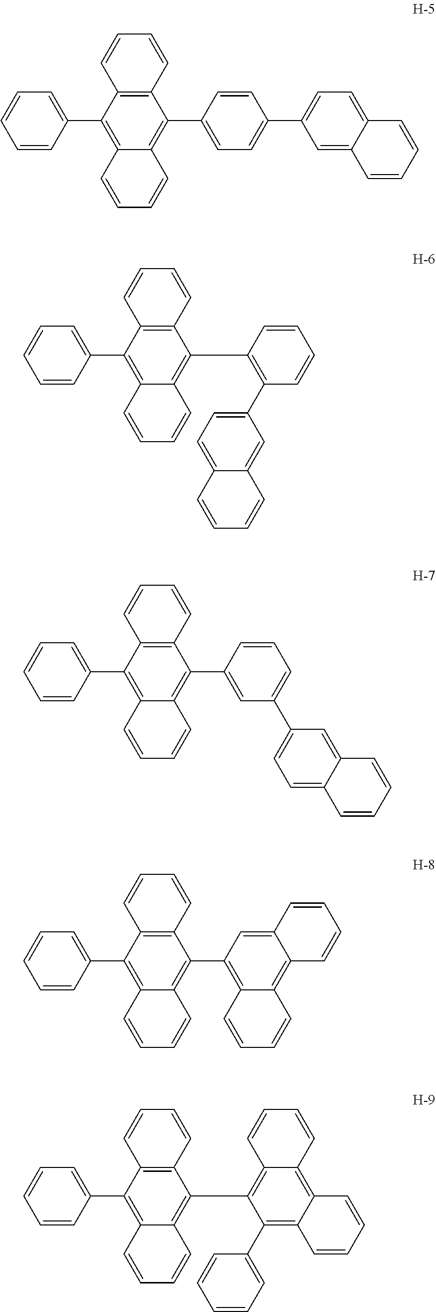 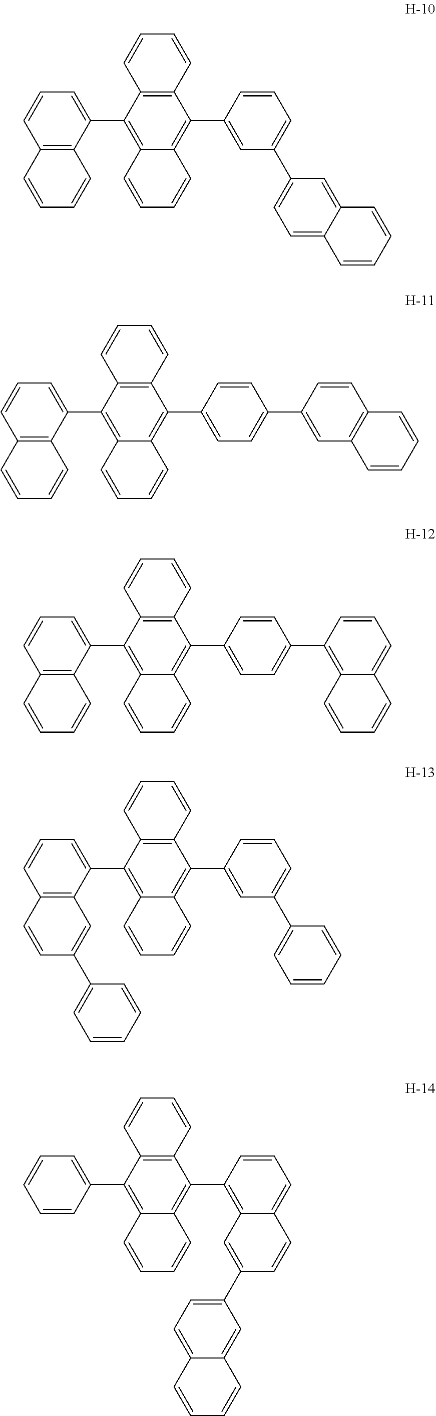 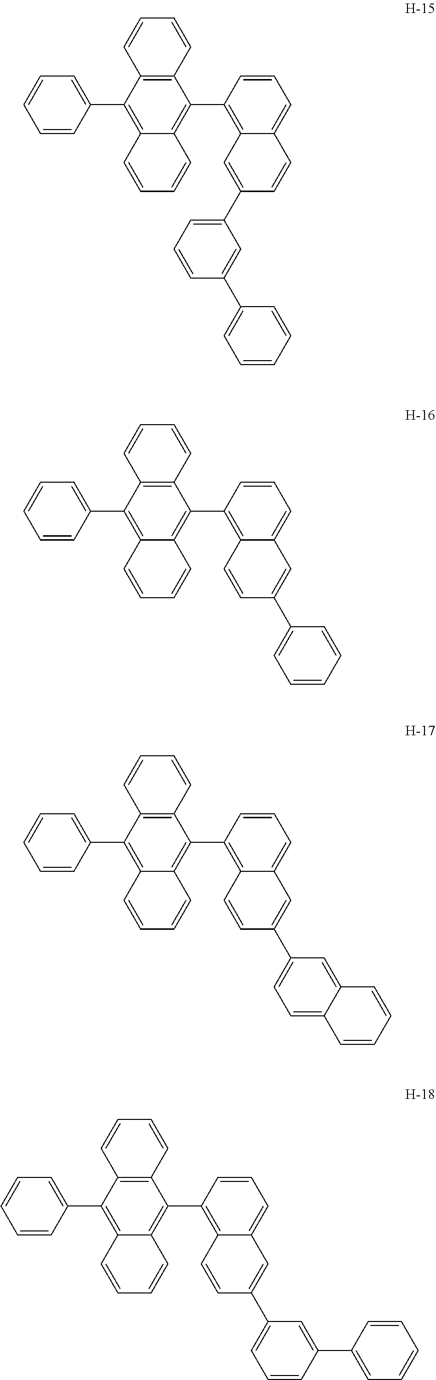 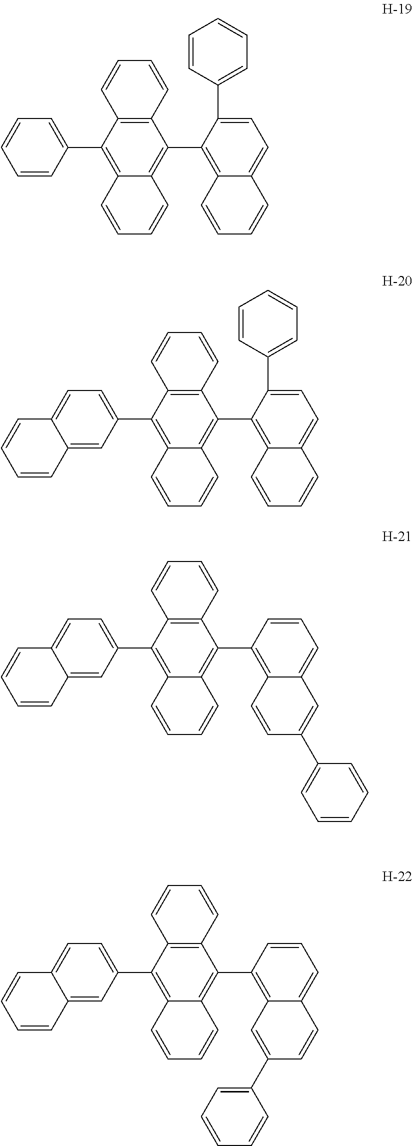 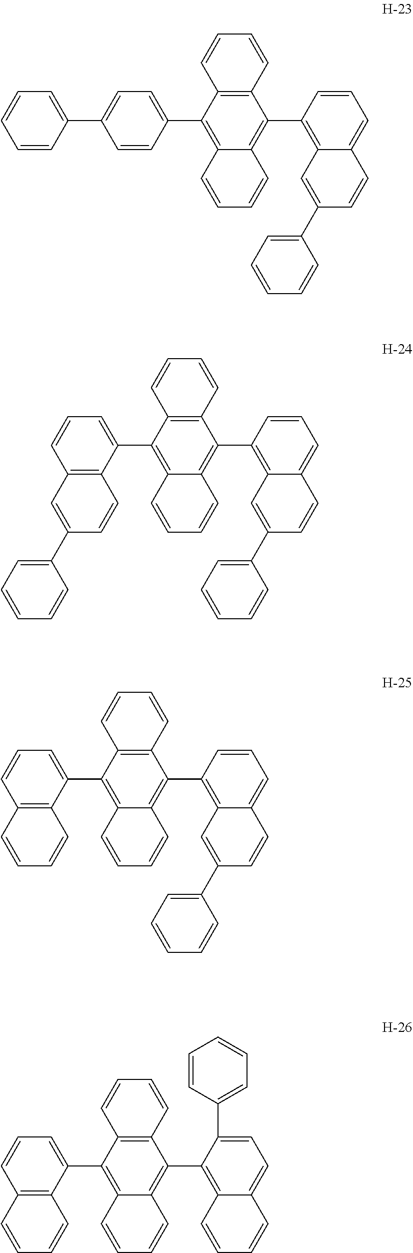 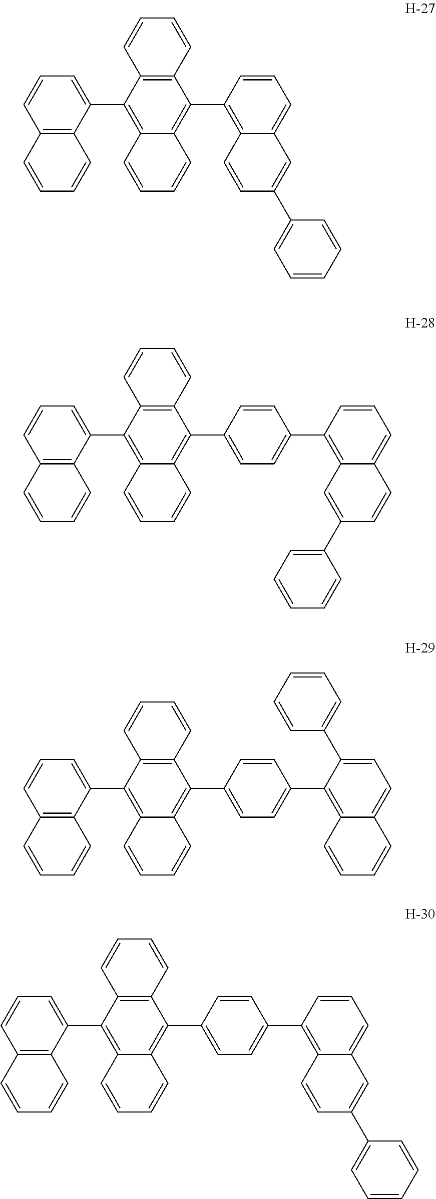 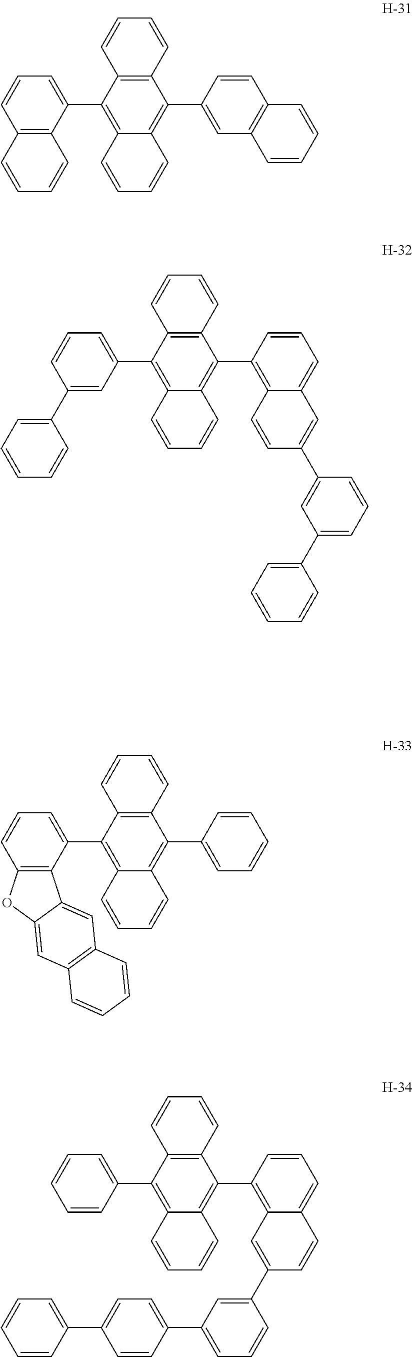 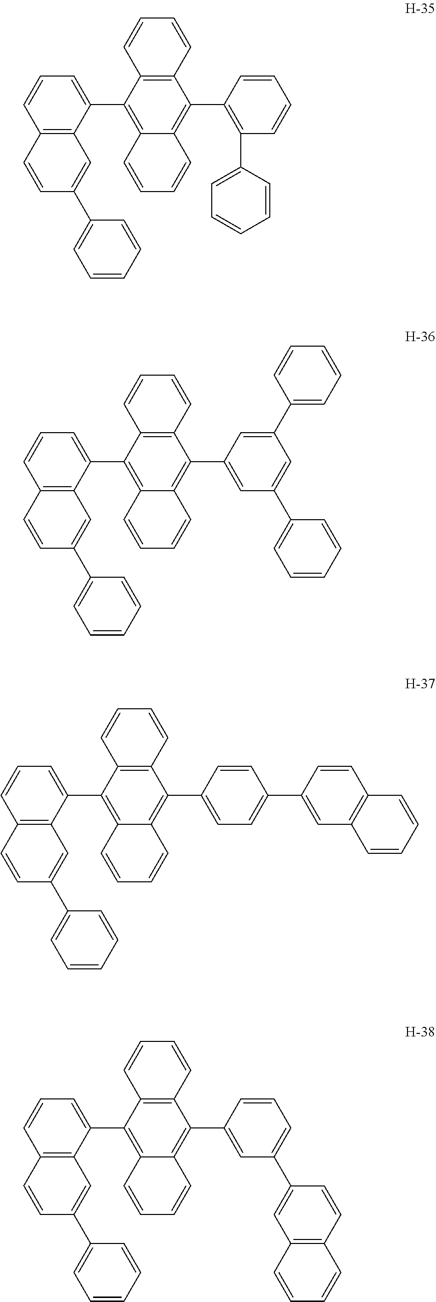 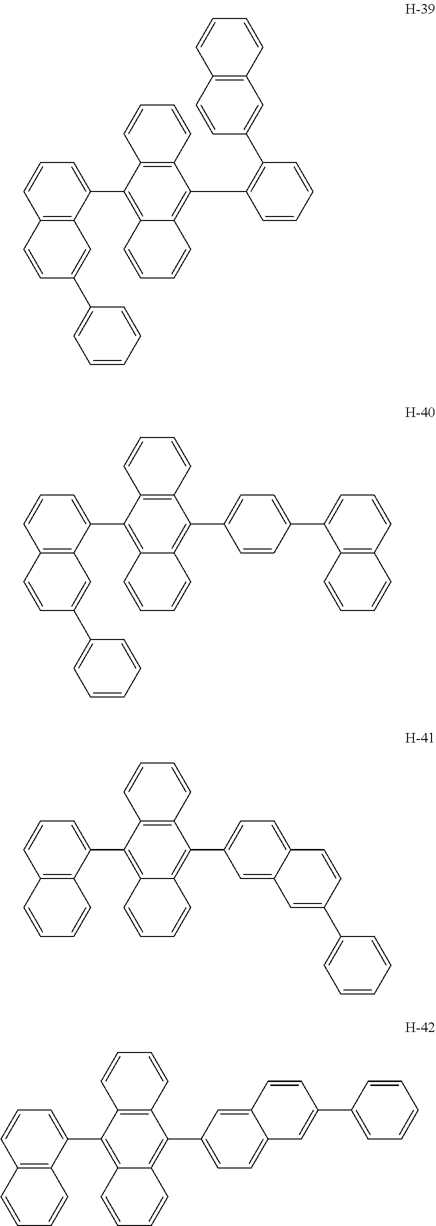 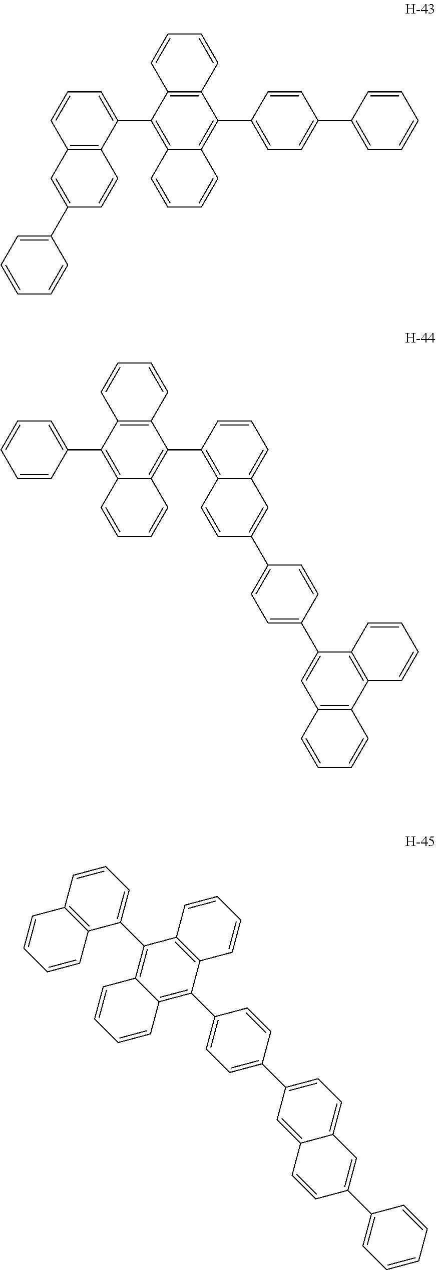 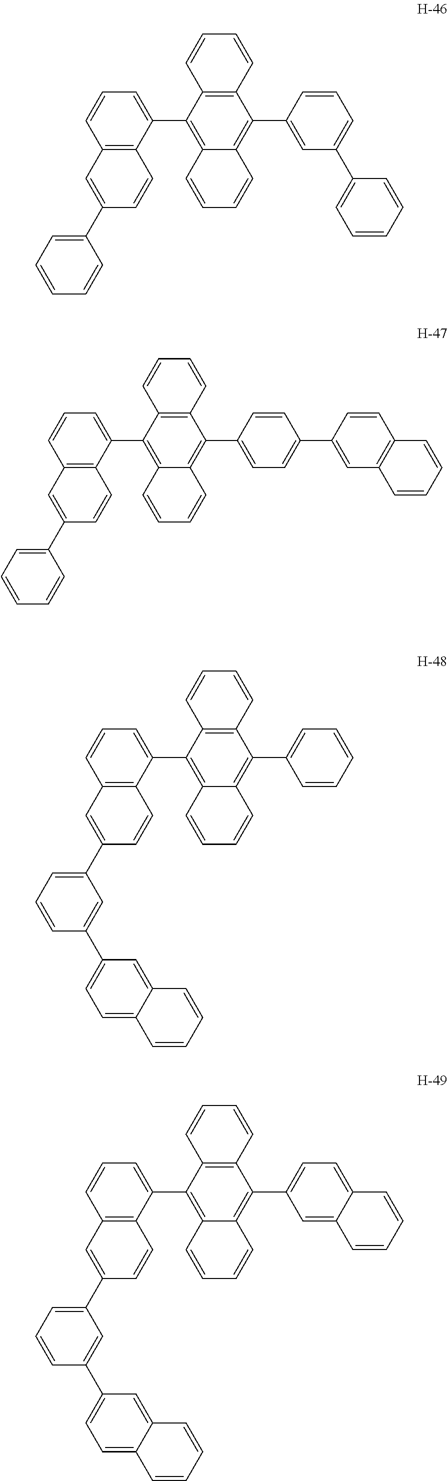 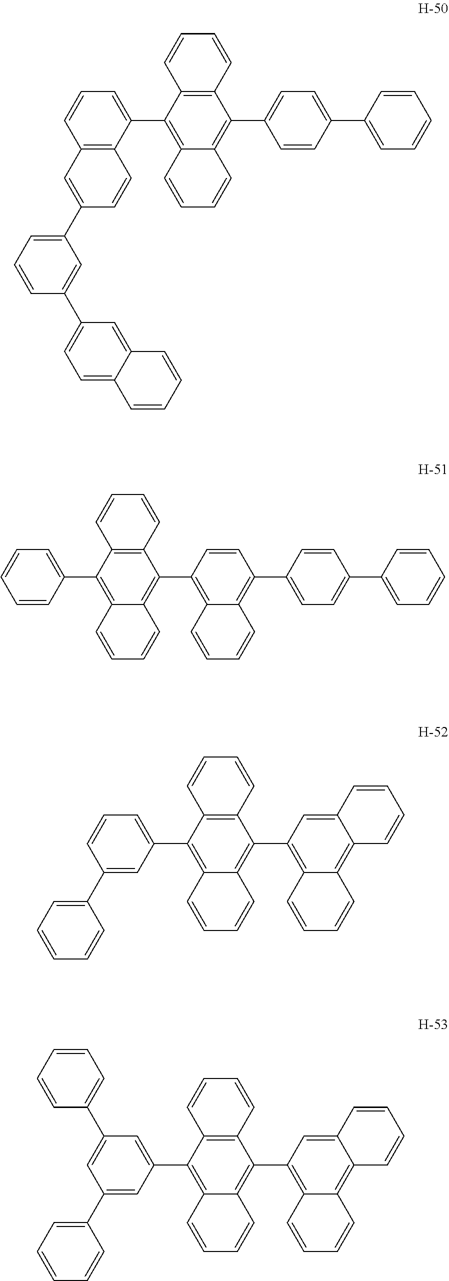 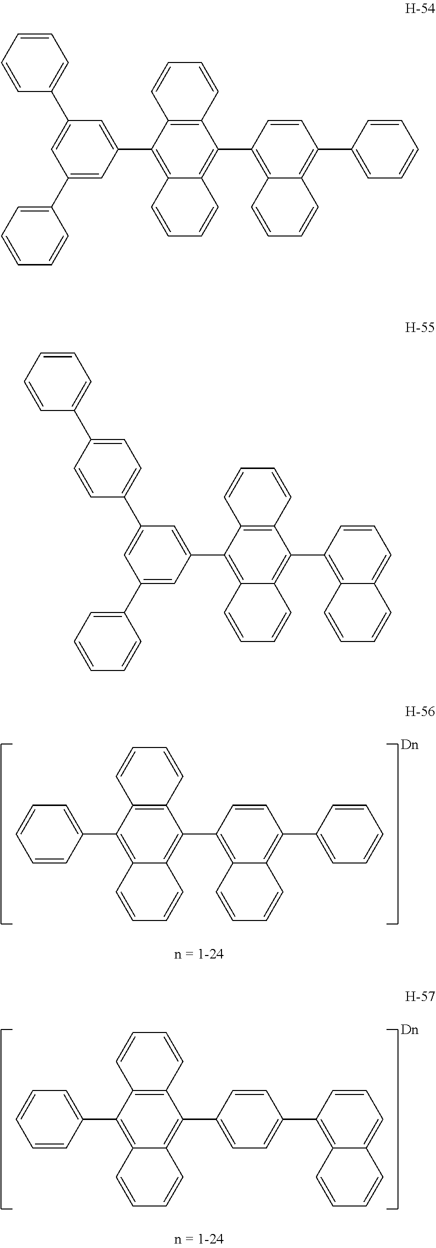 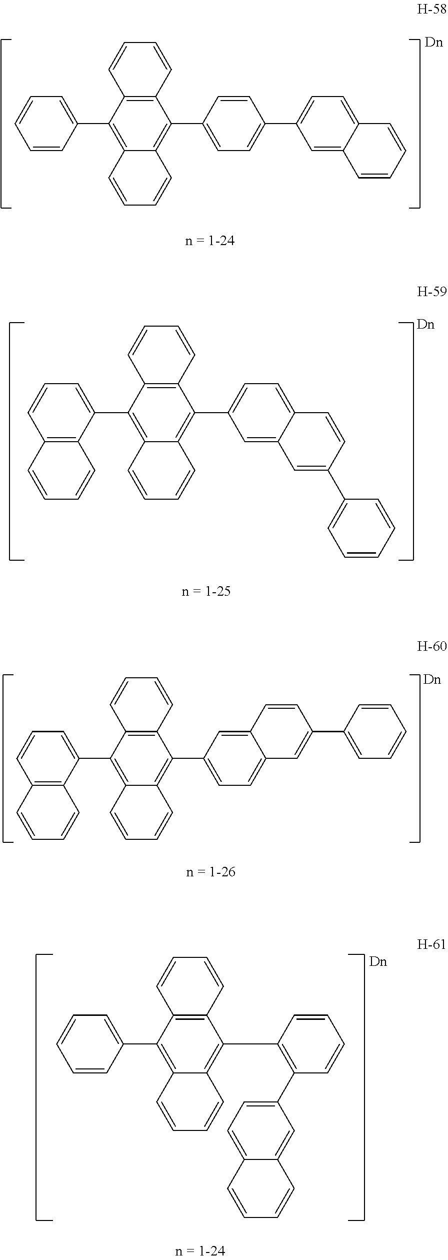 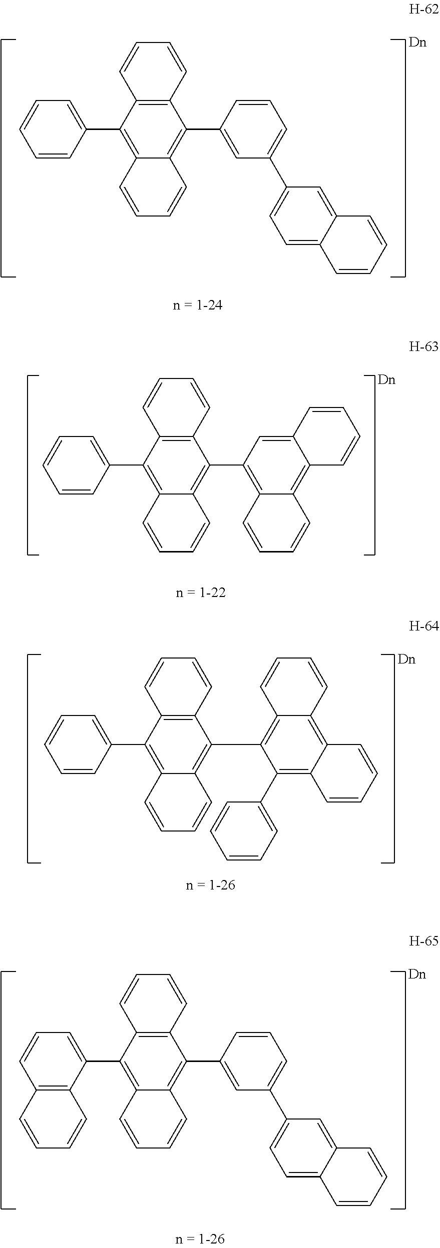 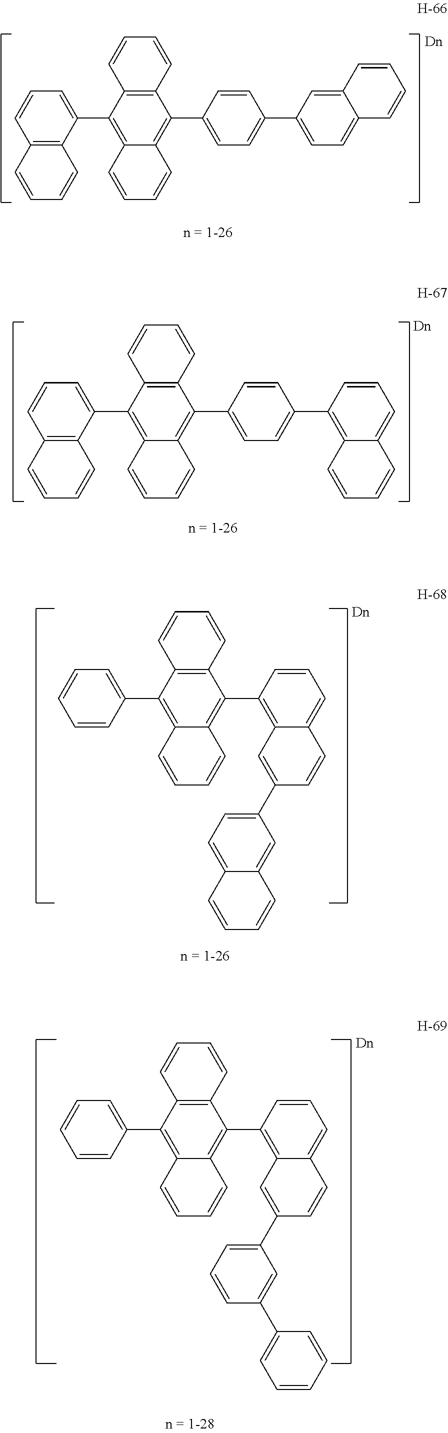 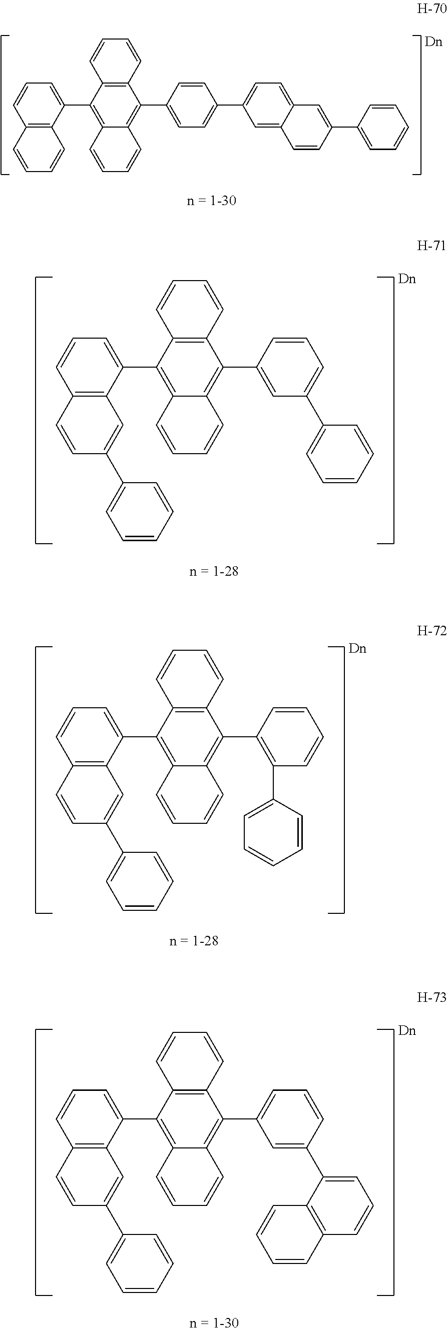 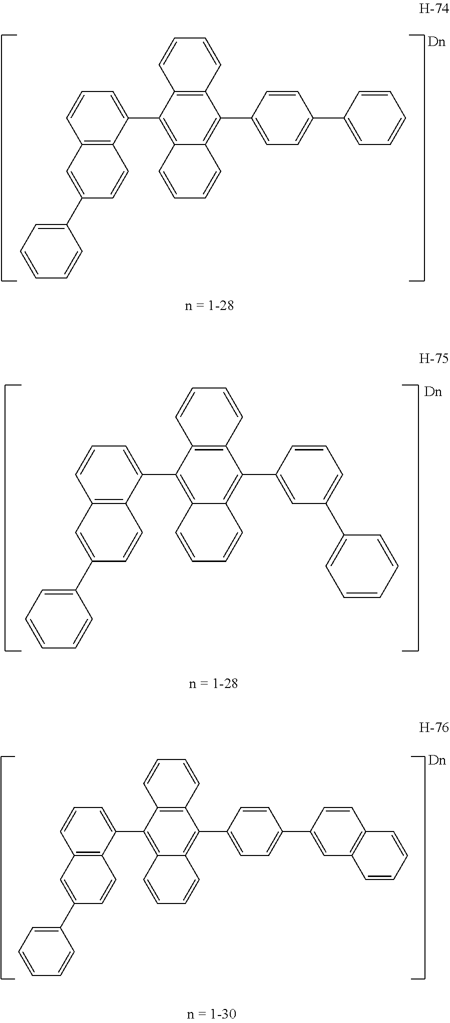 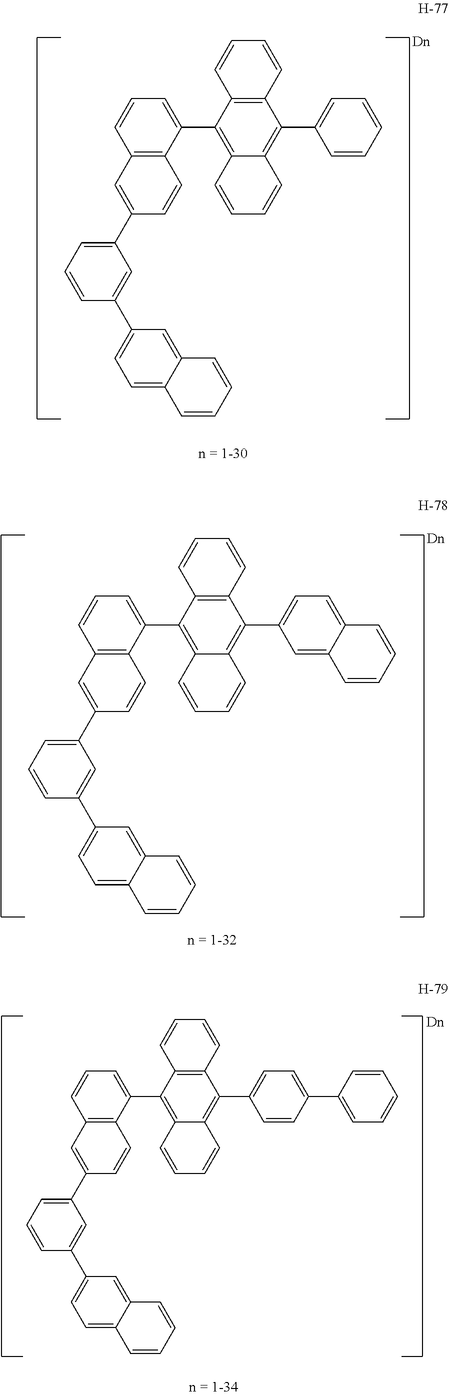 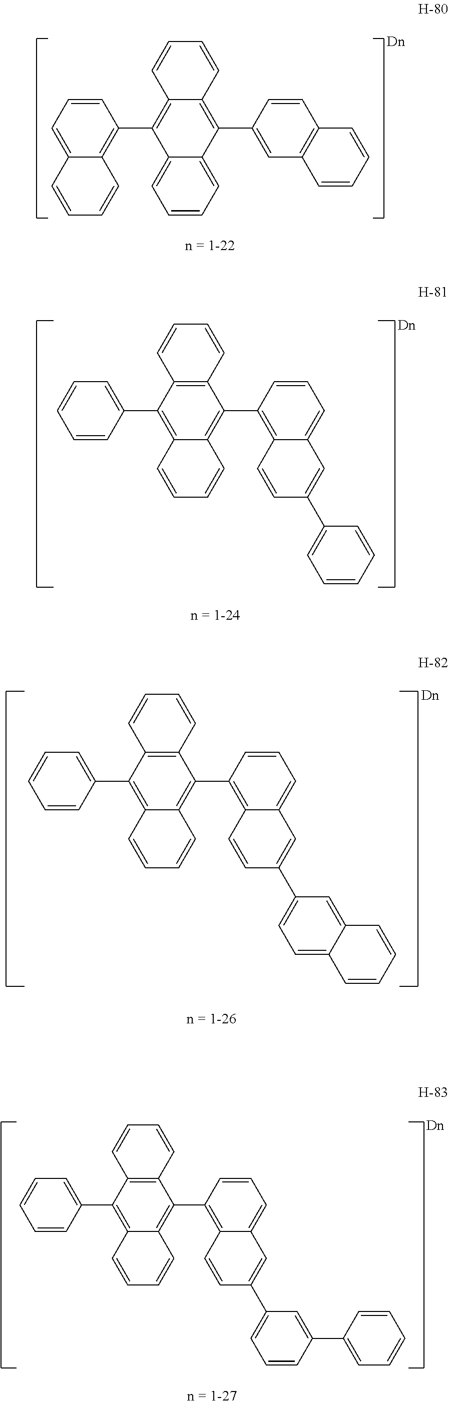 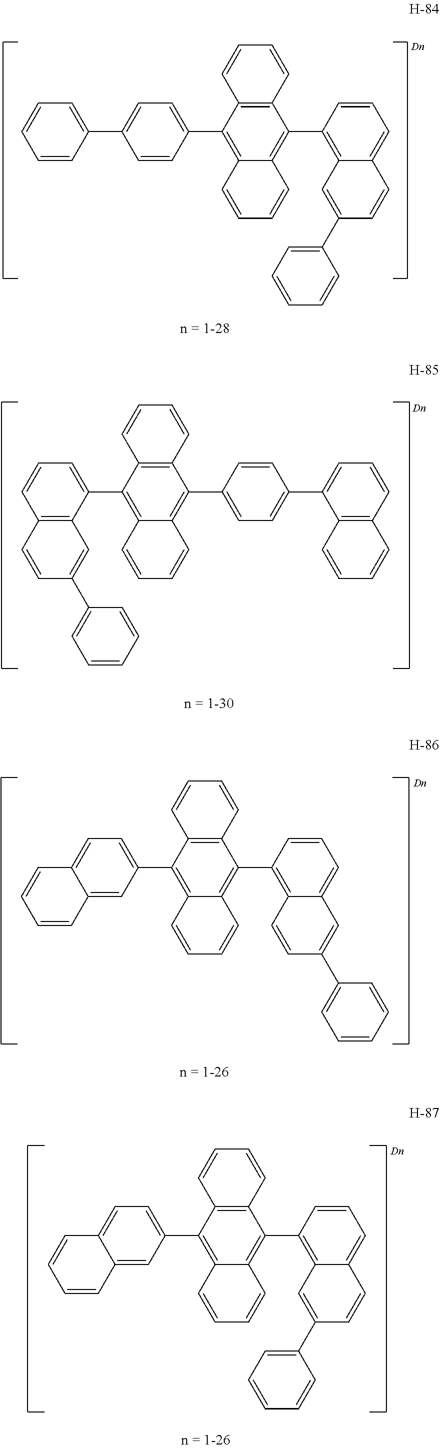 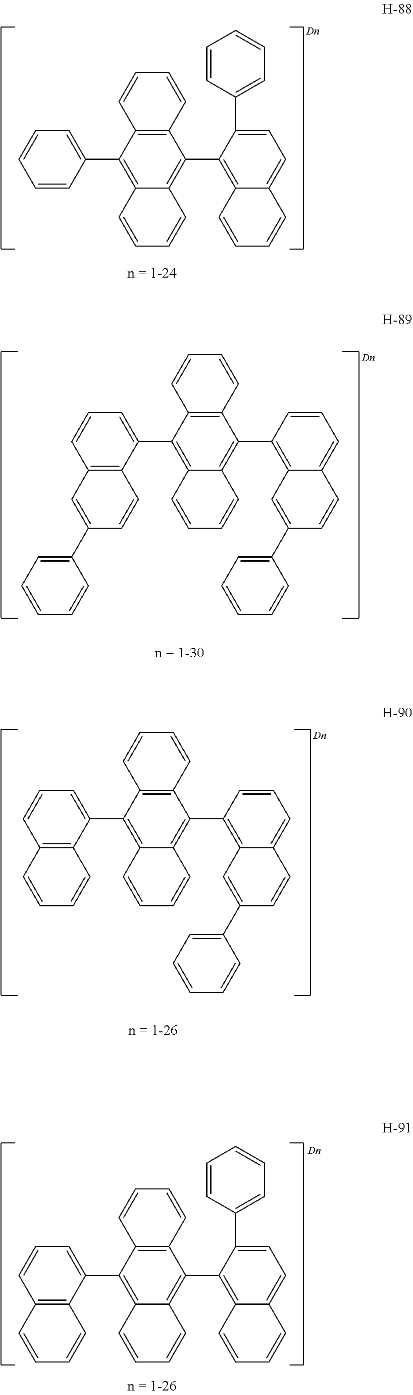 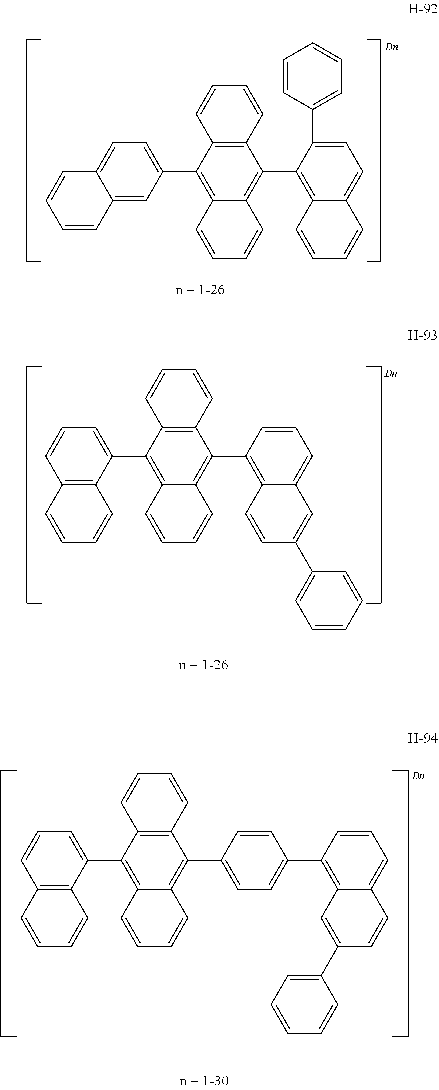 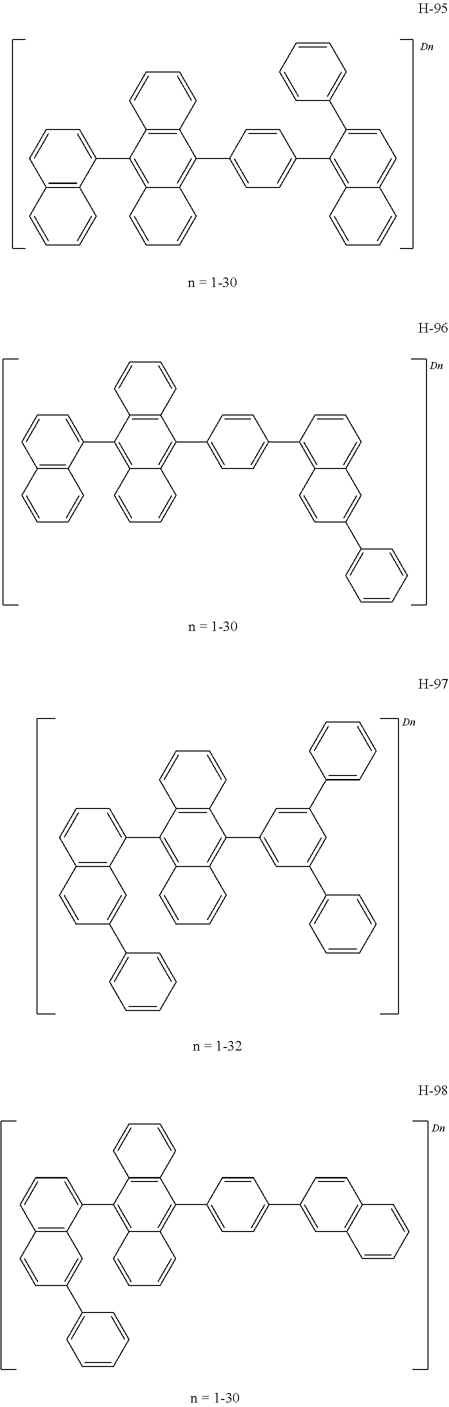 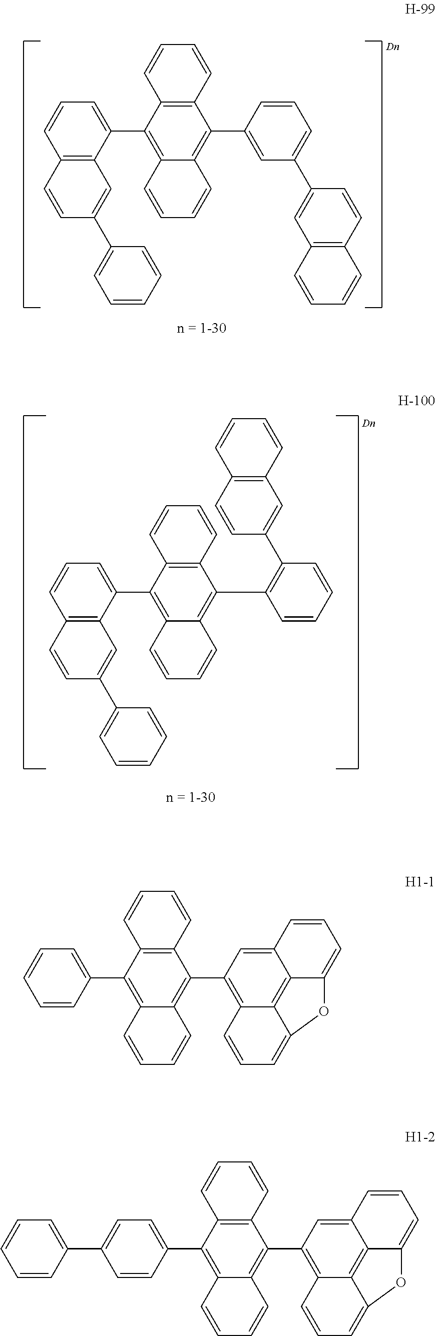 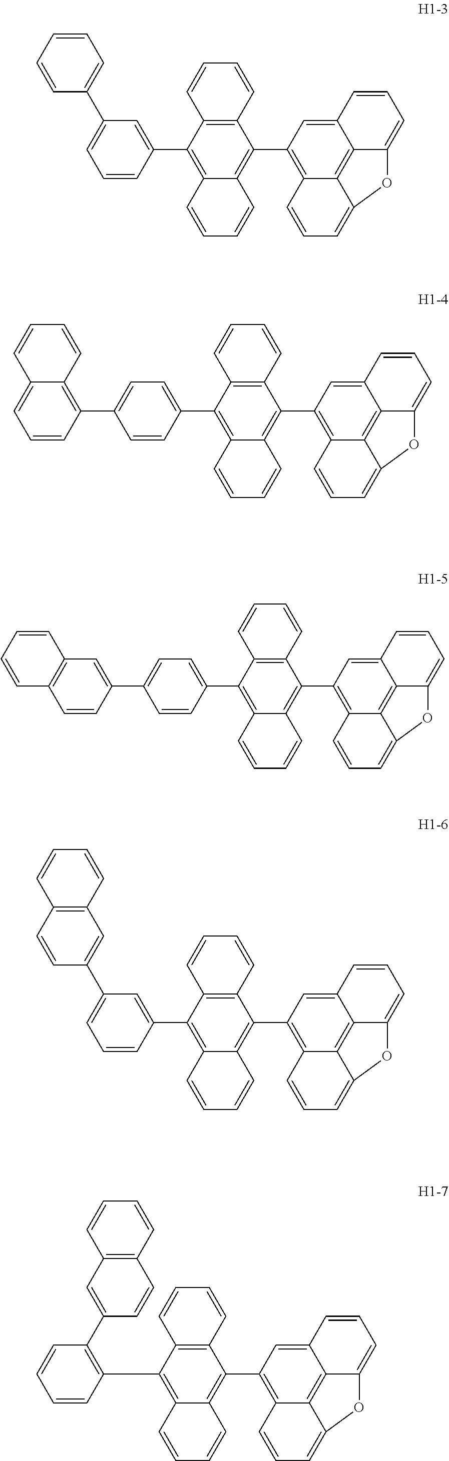 [SMILES: c1ccc(-c2cccc(-c3c4ccccc4c(-c4cc5cccc6oc7cccc4c7c56)c4ccccc34)c2)cc1 ; c1ccc2c(-c3ccc(-c4c5ccccc5c(-c5cc6cccc7oc8cccc5c8c67)c5ccccc45)cc3)cccc2c1 ; c1ccc2cc(-c3ccc(-c4c5ccccc5c(-c5cc6cccc7oc8cccc5c8c67)c5ccccc45)cc3)ccc2c1 ; c1cc(-c2ccc3ccccc3c2)cc(-c2c3ccccc3c(-c3cc4cccc5oc6cccc3c6c45)c3ccccc23)c1 ; c1ccc(-c2c3ccccc3c(-c3cc4cccc5oc6cccc3c6c45)c3ccccc23)c(-c2ccc3ccccc3c2)c1] 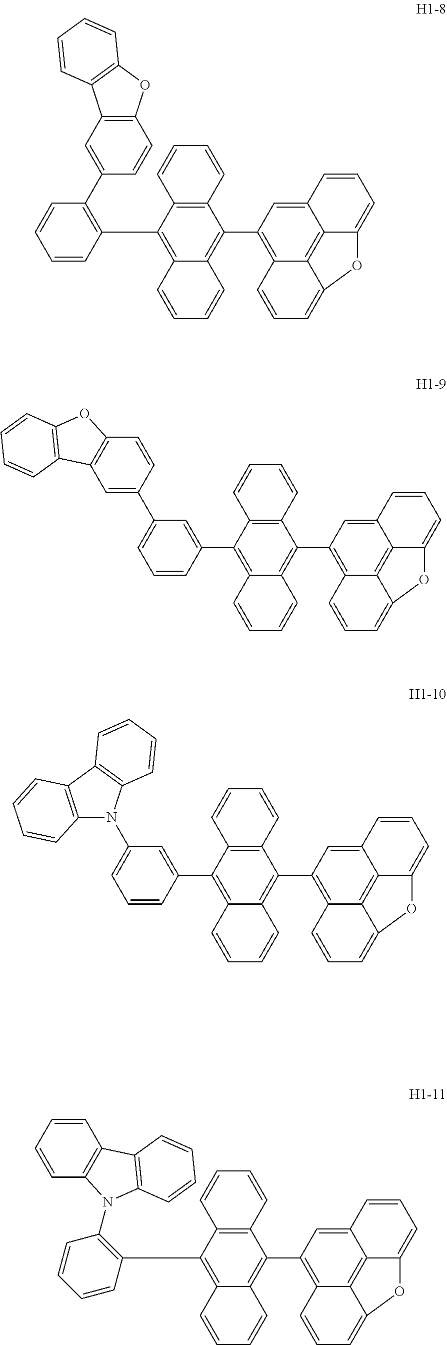 [SMILES: c1ccc(-c2c3ccccc3c(-c3cc4cccc5oc6cccc3c6c45)c3ccccc23)c(-c2ccc3oc4ccccc4c3c2)c1 ; c1cc(-c2ccc3oc4ccccc4c3c2)cc(-c2c3ccccc3c(-c3cc4cccc5oc6cccc3c6c45)c3ccccc23)c1 ; c1cc(-c2c3ccccc3c(-c3cc4cccc5oc6cccc3c6c45)c3ccccc23)cc(-n2c3ccccc3c3ccccc32)c1 ; c1ccc(-n2c3ccccc3c3ccccc32)c(-c2c3ccccc3c(-c3cc4cccc5oc6cccc3c6c45)c3ccccc23)c1] 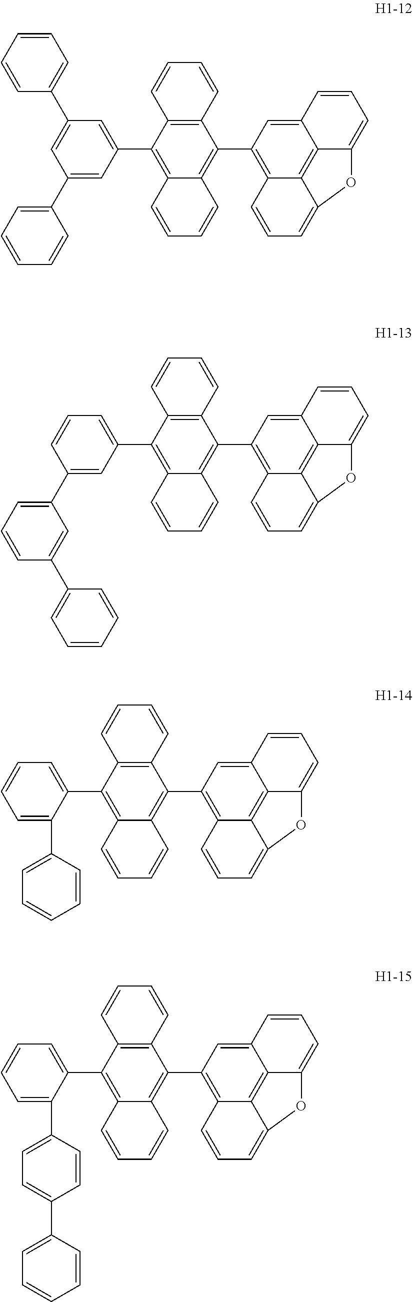 [SMILES: c1ccc(-c2cc(-c3ccccc3)cc(-c3c4ccccc4c(-c4cc5cccc6oc7cccc4c7c56)c4ccccc34)c2)cc1 ; c1ccc(-c2cccc(-c3cccc(-c4c5ccccc5c(-c5cc6cccc7oc8cccc5c8c67)c5ccccc45)c3)c2)cc1 ; c1ccc(-c2ccccc2-c2c3ccccc3c(-c3cc4cccc5oc6cccc3c6c45)c3ccccc23)cc1 ; c1ccc(-c2ccc(-c3ccccc3-c3c4ccccc4c(-c4cc5cccc6oc7cccc4c7c56)c4ccccc34)cc2)cc1] 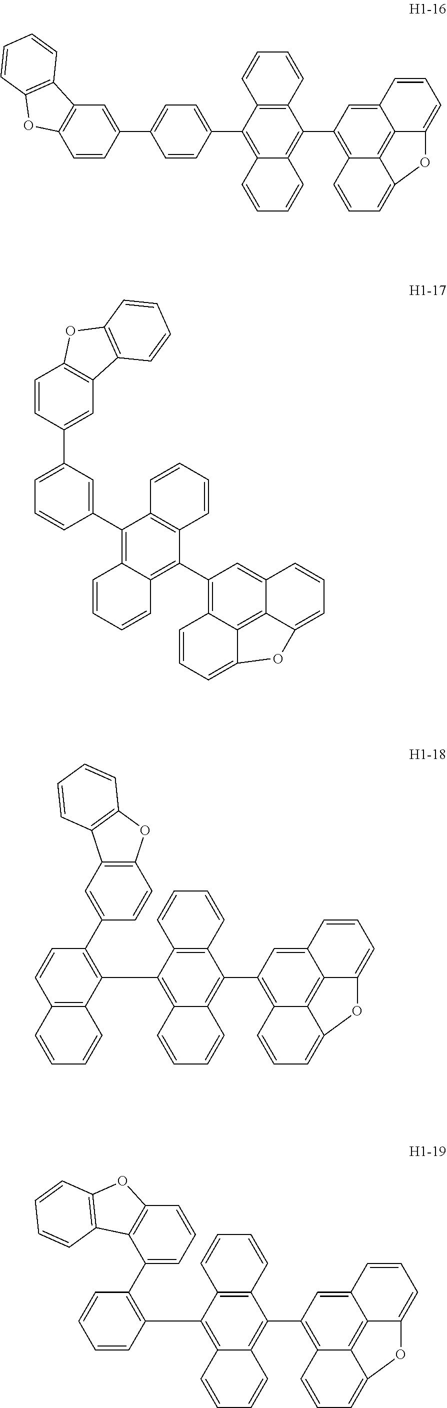 [SMILES: c1ccc2c(c1)oc1ccc(-c3ccc(-c4c5ccccc5c(-c5cc6cccc7oc8cccc5c8c67)c5ccccc45)cc3)cc12 ; c1cc(-c2ccc3oc4ccccc4c3c2)cc(-c2c3ccccc3c(-c3cc4cccc5oc6cccc3c6c45)c3ccccc23)c1 ; c1ccc2c(-c3c4ccccc4c(-c4cc5cccc6oc7cccc4c7c56)c4ccccc34)c(-c3ccc4oc5ccccc5c4c3)ccc2c1 ; c1ccc(-c2cccc3oc4ccccc4c23)c(-c2c3ccccc3c(-c3cc4cccc5oc6cccc3c6c45)c3ccccc23)c1] 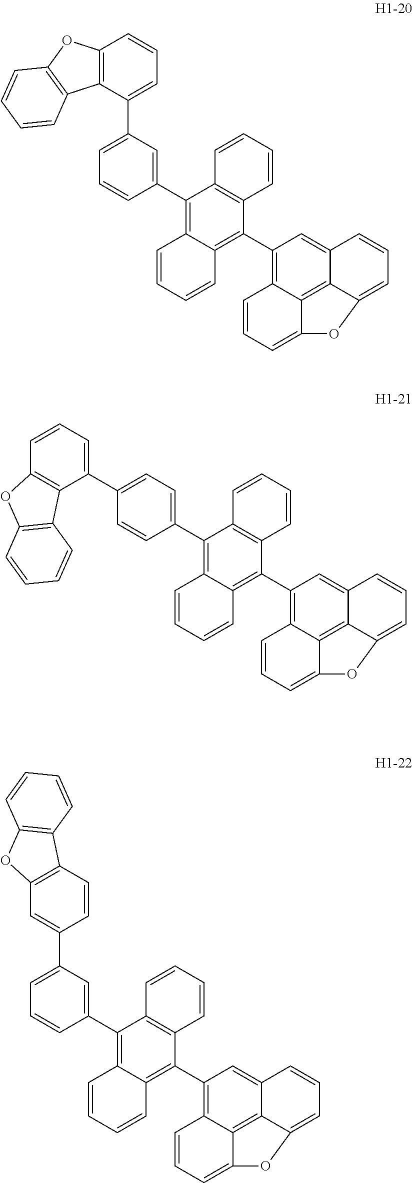 [SMILES: c1cc(-c2c3ccccc3c(-c3cc4cccc5oc6cccc3c6c45)c3ccccc23)cc(-c2cccc3oc4ccccc4c23)c1 ; c1ccc2c(c1)oc1cccc(-c3ccc(-c4c5ccccc5c(-c5cc6cccc7oc8cccc5c8c67)c5ccccc45)cc3)c12 ; c1cc(-c2ccc3c(c2)oc2ccccc23)cc(-c2c3ccccc3c(-c3cc4cccc5oc6cccc3c6c45)c3ccccc23)c1] 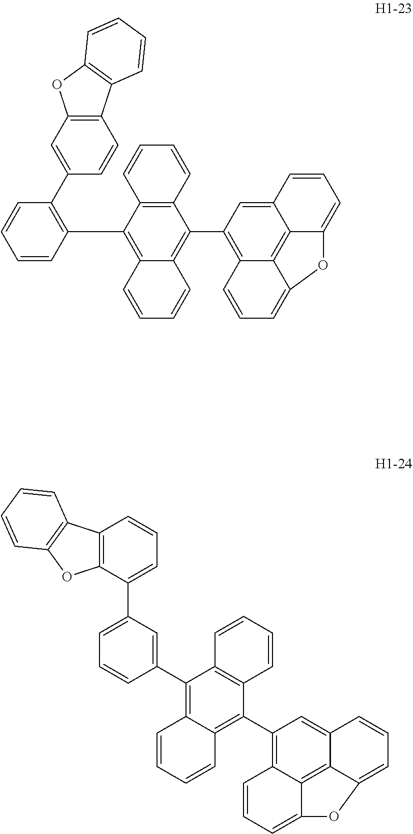 [SMILES: c1ccc(-c2c3ccccc3c(-c3cc4cccc5oc6cccc3c6c45)c3ccccc23)c(-c2ccc3c(c2)oc2ccccc23)c1 ; c1cc(-c2c3ccccc3c(-c3cc4cccc5oc6cccc3c6c45)c3ccccc23)cc(-c2cccc3c2oc2ccccc23)c1] 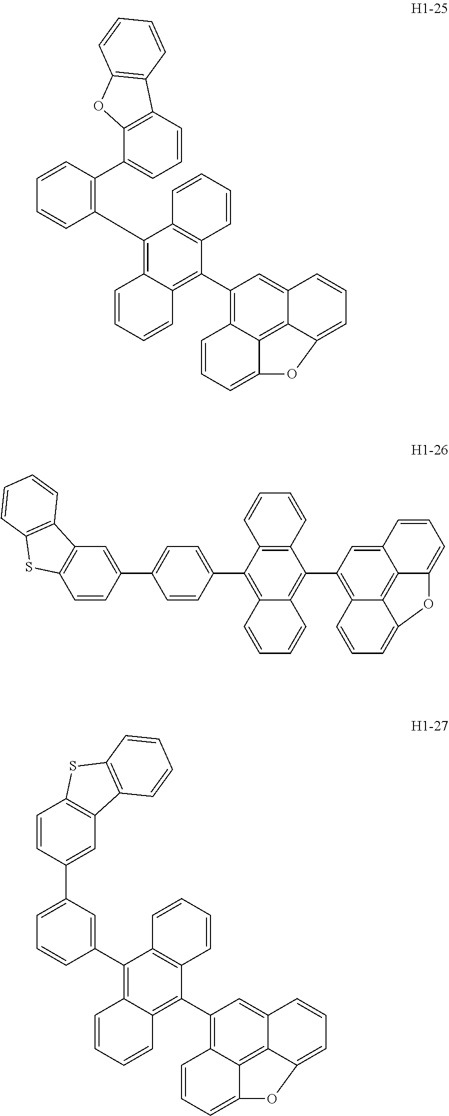 [SMILES: c1ccc(-c2cccc3c2oc2ccccc23)c(-c2c3ccccc3c(-c3cc4cccc5oc6cccc3c6c45)c3ccccc23)c1 ; c1cc2cc(-c3c4ccccc4c(-c4ccc(-c5ccc6sc7ccccc7c6c5)cc4)c4ccccc34)c3cccc4oc(c1)c2c43 ; c1cc(-c2ccc3sc4ccccc4c3c2)cc(-c2c3ccccc3c(-c3cc4cccc5oc6cccc3c6c45)c3ccccc23)c1] 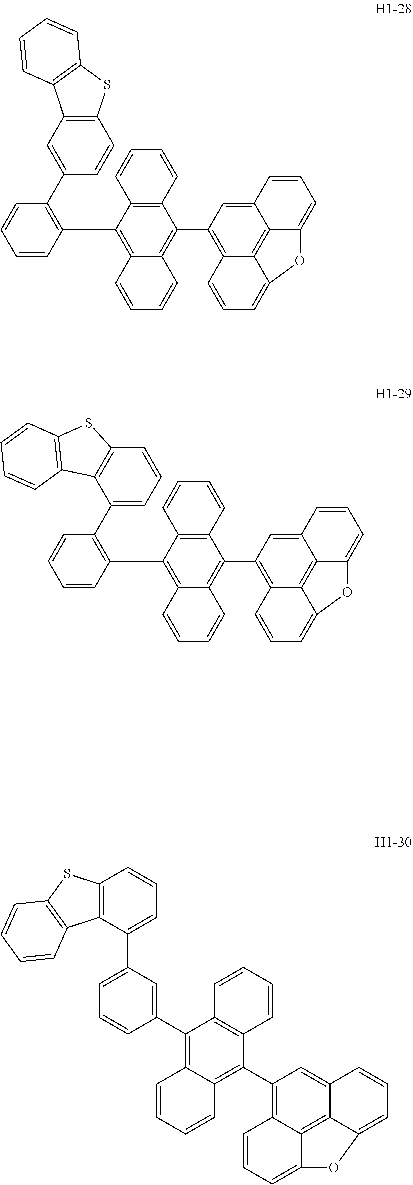 [SMILES: c1ccc(-c2c3ccccc3c(-c3cc4cccc5oc6cccc3c6c45)c3ccccc23)c(-c2ccc3sc4ccccc4c3c2)c1 ; c1ccc(-c2cccc3sc4ccccc4c23)c(-c2c3ccccc3c(-c3cc4cccc5oc6cccc3c6c45)c3ccccc23)c1 ; c1cc(-c2c3ccccc3c(-c3cc4cccc5oc6cccc3c6c45)c3ccccc23)cc(-c2cccc3sc4ccccc4c23)c1] 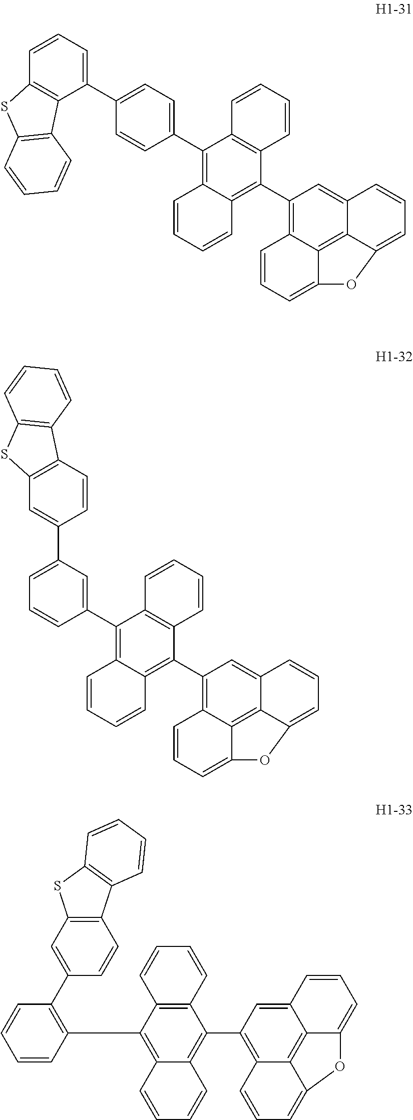 [SMILES: c1cc2cc(-c3c4ccccc4c(-c4ccc(-c5cccc6sc7ccccc7c56)cc4)c4ccccc34)c3cccc4oc(c1)c2c43 ; c1cc(-c2ccc3c(c2)sc2ccccc23)cc(-c2c3ccccc3c(-c3cc4cccc5oc6cccc3c6c45)c3ccccc23)c1 ; c1ccc(-c2c3ccccc3c(-c3cc4cccc5oc6cccc3c6c45)c3ccccc23)c(-c2ccc3c(c2)sc2ccccc23)c1] 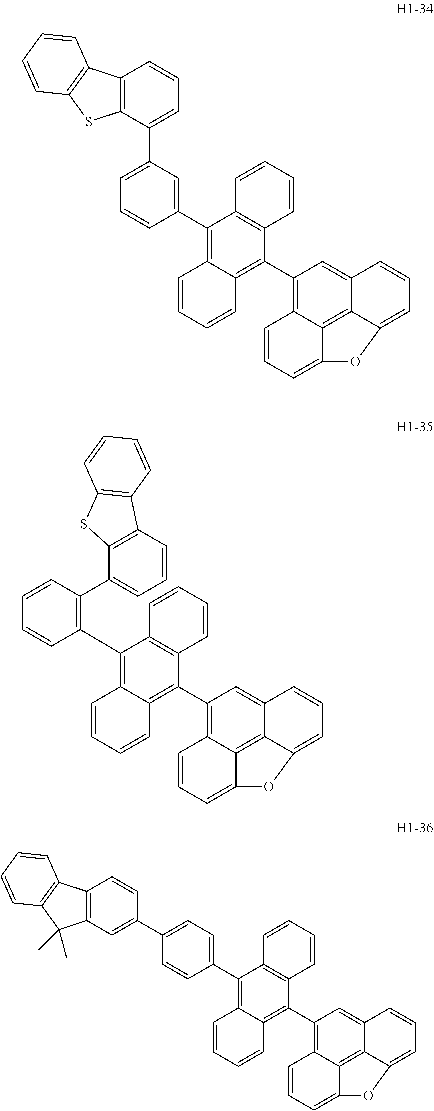 [SMILES: c1cc(-c2c3ccccc3c(-c3cc4cccc5oc6cccc3c6c45)c3ccccc23)cc(-c2cccc3c2sc2ccccc23)c1 ; c1ccc(-c2cccc3c2sc2ccccc23)c(-c2c3ccccc3c(-c3cc4cccc5oc6cccc3c6c45)c3ccccc23)c1 ; CC1(C)c2ccccc2-c2ccc(-c3ccc(-c4c5ccccc5c(-c5cc6cccc7oc8cccc5c8c67)c5ccccc45)cc3)cc21] 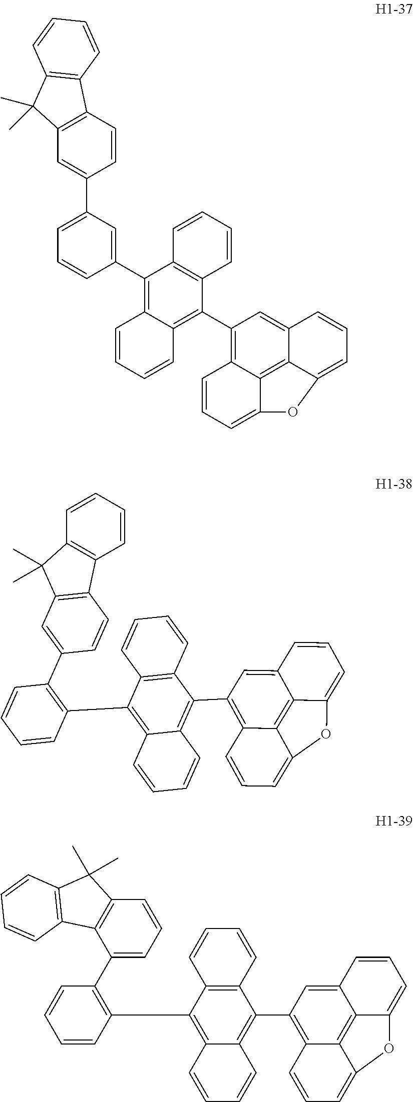 [SMILES: CC1(C)c2ccccc2-c2ccc(-c3cccc(-c4c5ccccc5c(-c5cc6cccc7oc8cccc5c8c67)c5ccccc45)c3)cc21 ; CC1(C)c2ccccc2-c2ccc(-c3ccccc3-c3c4ccccc4c(-c4cc5cccc6oc7cccc4c7c56)c4ccccc34)cc21 ; CC1(C)c2ccccc2-c2c(-c3ccccc3-c3c4ccccc4c(-c4cc5cccc6oc7cccc4c7c56)c4ccccc34)cccc21] 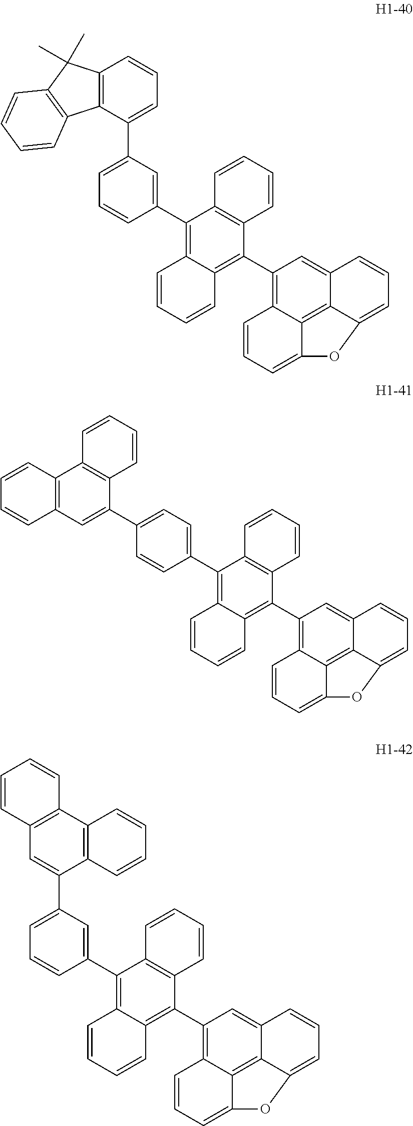 [SMILES: CC1(C)c2ccccc2-c2c(-c3cccc(-c4c5ccccc5c(-c5cc6cccc7oc8cccc5c8c67)c5ccccc45)c3)cccc21 ; c1ccc2c(c1)cc(-c1ccc(-c3c4ccccc4c(-c4cc5cccc6oc7cccc4c7c56)c4ccccc34)cc1)c1ccccc12 ; c1cc(-c2c3ccccc3c(-c3cc4cccc5oc6cccc3c6c45)c3ccccc23)cc(-c2cc3ccccc3c3ccccc23)c1] 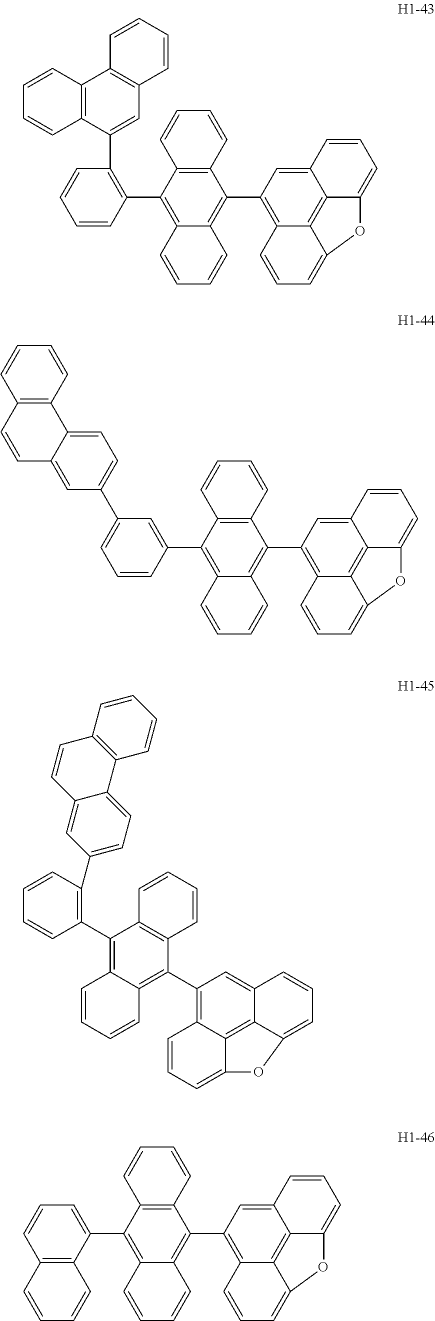 [SMILES: c1ccc(-c2cc3ccccc3c3ccccc23)c(-c2c3ccccc3c(-c3cc4cccc5oc6cccc3c6c45)c3ccccc23)c1 ; c1cc(-c2ccc3c(ccc4ccccc43)c2)cc(-c2c3ccccc3c(-c3cc4cccc5oc6cccc3c6c45)c3ccccc23)c1 ; c1ccc(-c2c3ccccc3c(-c3cc4cccc5oc6cccc3c6c45)c3ccccc23)c(-c2ccc3c(ccc4ccccc43)c2)c1 ; c1ccc2c(-c3c4ccccc4c(-c4cc5cccc6oc7cccc4c7c56)c4ccccc34)cccc2c1] 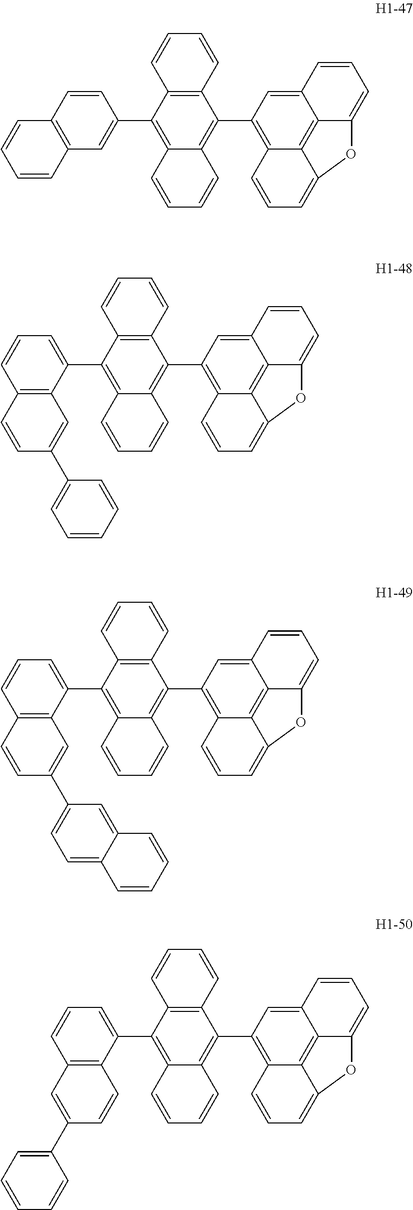 [SMILES: c1ccc2cc(-c3c4ccccc4c(-c4cc5cccc6oc7cccc4c7c56)c4ccccc34)ccc2c1 ; c1ccc(-c2ccc3cccc(-c4c5ccccc5c(-c5cc6cccc7oc8cccc5c8c67)c5ccccc45)c3c2)cc1 ; c1ccc2cc(-c3ccc4cccc(-c5c6ccccc6c(-c6cc7cccc8oc9cccc6c9c78)c6ccccc56)c4c3)ccc2c1 ; c1ccc(-c2ccc3c(-c4c5ccccc5c(-c5cc6cccc7oc8cccc5c8c67)c5ccccc45)cccc3c2)cc1] 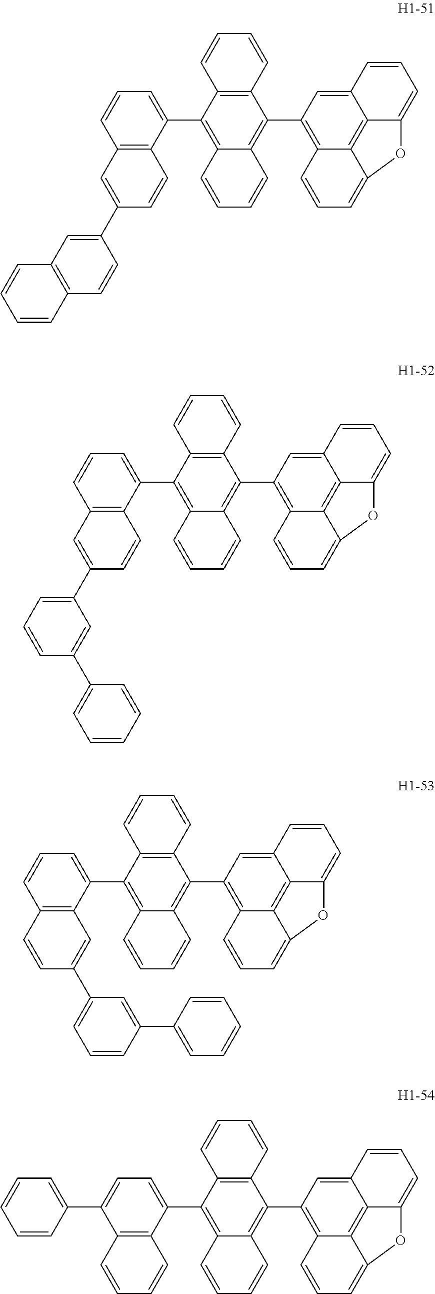 [SMILES: c1ccc2cc(-c3ccc4c(-c5c6ccccc6c(-c6cc7cccc8oc9cccc6c9c78)c6ccccc56)cccc4c3)ccc2c1 ; c1ccc(-c2cccc(-c3ccc4c(-c5c6ccccc6c(-c6cc7cccc8oc9cccc6c9c78)c6ccccc56)cccc4c3)c2)cc1 ; c1ccc(-c2cccc(-c3ccc4cccc(-c5c6ccccc6c(-c6cc7cccc8oc9cccc6c9c78)c6ccccc56)c4c3)c2)cc1 ; c1ccc(-c2ccc(-c3c4ccccc4c(-c4cc5cccc6oc7cccc4c7c56)c4ccccc34)c3ccccc23)cc1] 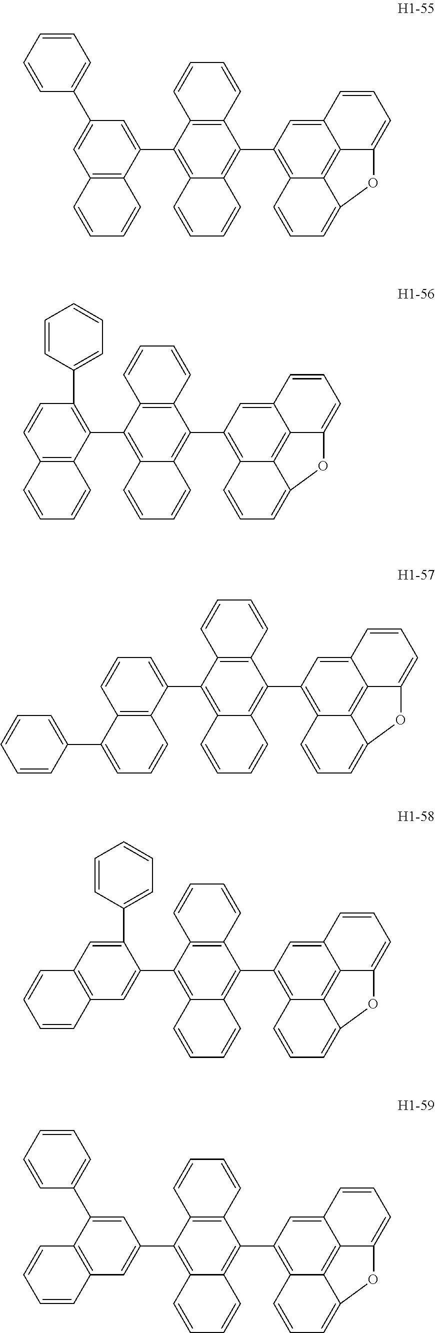 [SMILES: c1ccc(-c2cc(-c3c4ccccc4c(-c4cc5cccc6oc7cccc4c7c56)c4ccccc34)c3ccccc3c2)cc1 ; c1ccc(-c2ccc3ccccc3c2-c2c3ccccc3c(-c3cc4cccc5oc6cccc3c6c45)c3ccccc23)cc1 ; c1ccc(-c2cccc3c(-c4c5ccccc5c(-c5cc6cccc7oc8cccc5c8c67)c5ccccc45)cccc23)cc1 ; c1ccc(-c2cc3ccccc3cc2-c2c3ccccc3c(-c3cc4cccc5oc6cccc3c6c45)c3ccccc23)cc1 ; c1ccc(-c2cc(-c3c4ccccc4c(-c4cc5cccc6oc7cccc4c7c56)c4ccccc34)cc3ccccc23)cc1] 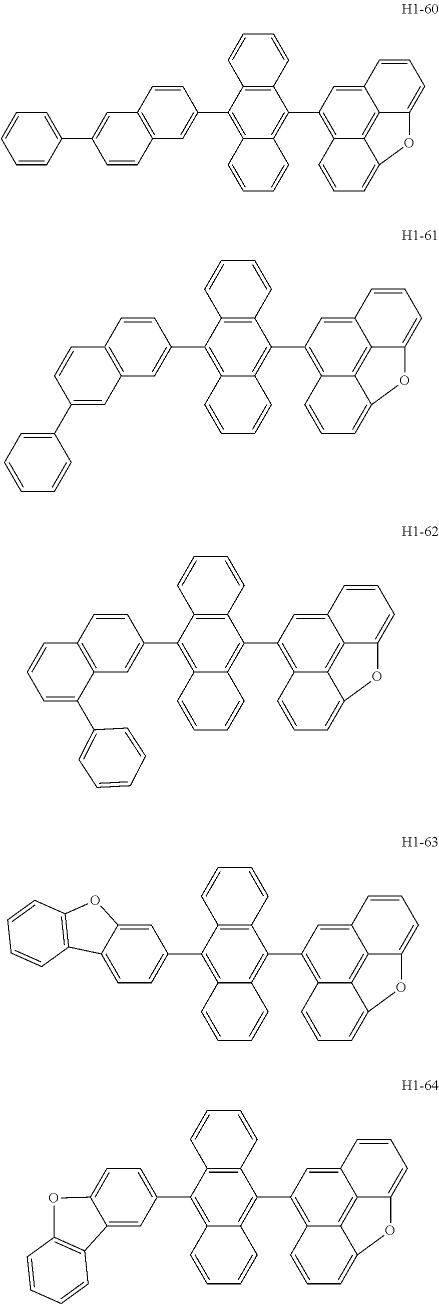 [SMILES: c1ccc(-c2ccc3cc(-c4c5ccccc5c(-c5cc6cccc7oc8cccc5c8c67)c5ccccc45)ccc3c2)cc1 ; c1ccc(-c2ccc3ccc(-c4c5ccccc5c(-c5cc6cccc7oc8cccc5c8c67)c5ccccc45)cc3c2)cc1 ; c1ccc(-c2cccc3ccc(-c4c5ccccc5c(-c5cc6cccc7oc8cccc5c8c67)c5ccccc45)cc23)cc1 ; c1ccc2c(c1)oc1cc(-c3c4ccccc4c(-c4cc5cccc6oc7cccc4c7c56)c4ccccc34)ccc12 ; c1ccc2c(c1)oc1ccc(-c3c4ccccc4c(-c4cc5cccc6oc7cccc4c7c56)c4ccccc34)cc12] 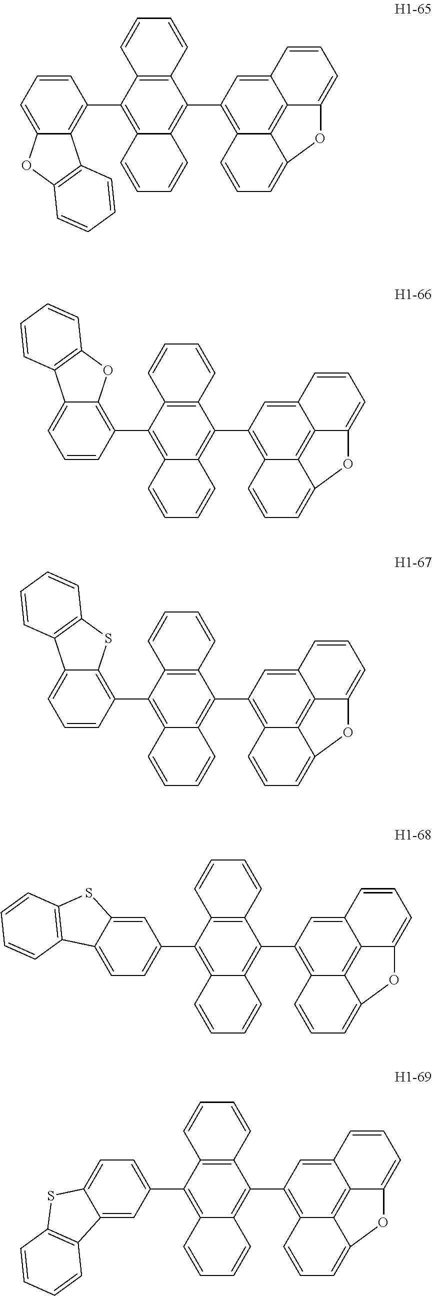 [SMILES: c1ccc2c(c1)oc1cccc(-c3c4ccccc4c(-c4cc5cccc6oc7cccc4c7c56)c4ccccc34)c12 ; c1ccc2c(c1)oc1c(-c3c4ccccc4c(-c4cc5cccc6oc7cccc4c7c56)c4ccccc34)cccc12 ; c1cc2cc(-c3c4ccccc4c(-c4cccc5c4sc4ccccc45)c4ccccc34)c3cccc4oc(c1)c2c43 ; c1cc2cc(-c3c4ccccc4c(-c4ccc5c(c4)sc4ccccc45)c4ccccc34)c3cccc4oc(c1)c2c43 ; c1cc2cc(-c3c4ccccc4c(-c4ccc5sc6ccccc6c5c4)c4ccccc34)c3cccc4oc(c1)c2c43] 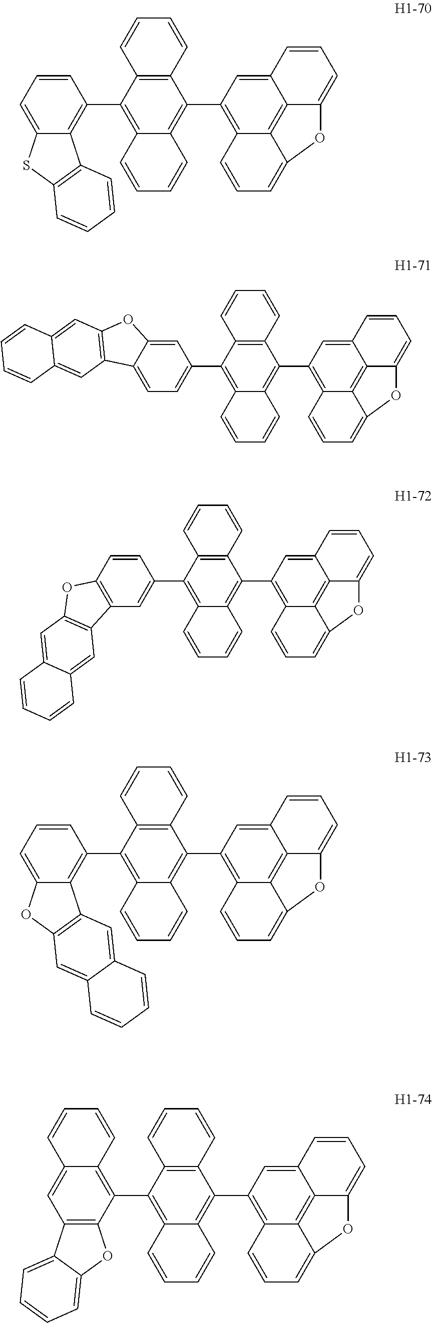 [SMILES: c1cc2cc(-c3c4ccccc4c(-c4cccc5sc6ccccc6c45)c4ccccc34)c3cccc4oc(c1)c2c43 ; c1ccc2cc3c(cc2c1)oc1cc(-c2c4ccccc4c(-c4cc5cccc6oc7cccc4c7c56)c4ccccc24)ccc13 ; c1ccc2cc3c(cc2c1)oc1ccc(-c2c4ccccc4c(-c4cc5cccc6oc7cccc4c7c56)c4ccccc24)cc13 ; c1ccc2cc3c(cc2c1)oc1cccc(-c2c4ccccc4c(-c4cc5cccc6oc7cccc4c7c56)c4ccccc24)c13 ; c1ccc2c(-c3c4ccccc4c(-c4cc5cccc6oc7cccc4c7c56)c4ccccc34)c3oc4ccccc4c3cc2c1] 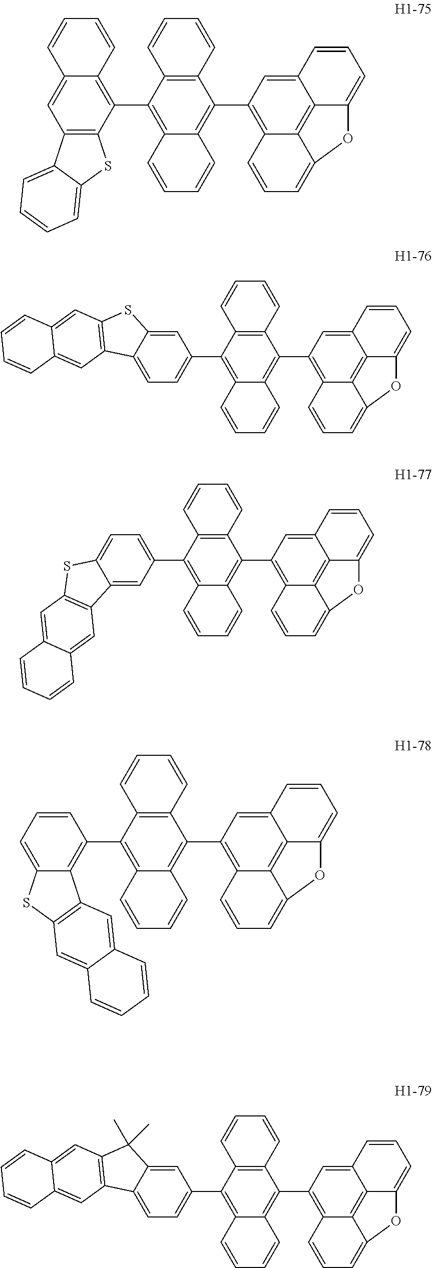 [SMILES: c1ccc2c(-c3c4ccccc4c(-c4cc5cccc6oc7cccc4c7c56)c4ccccc34)c3sc4ccccc4c3cc2c1 ; c1ccc2cc3c(cc2c1)sc1cc(-c2c4ccccc4c(-c4cc5cccc6oc7cccc4c7c56)c4ccccc24)ccc13 ; c1ccc2cc3c(cc2c1)sc1ccc(-c2c4ccccc4c(-c4cc5cccc6oc7cccc4c7c56)c4ccccc24)cc13 ; c1ccc2cc3c(cc2c1)sc1cccc(-c2c4ccccc4c(-c4cc5cccc6oc7cccc4c7c56)c4ccccc24)c13 ; CC1(C)c2cc(-c3c4ccccc4c(-c4cc5cccc6oc7cccc4c7c56)c4ccccc34)ccc2-c2cc3ccccc3cc21] 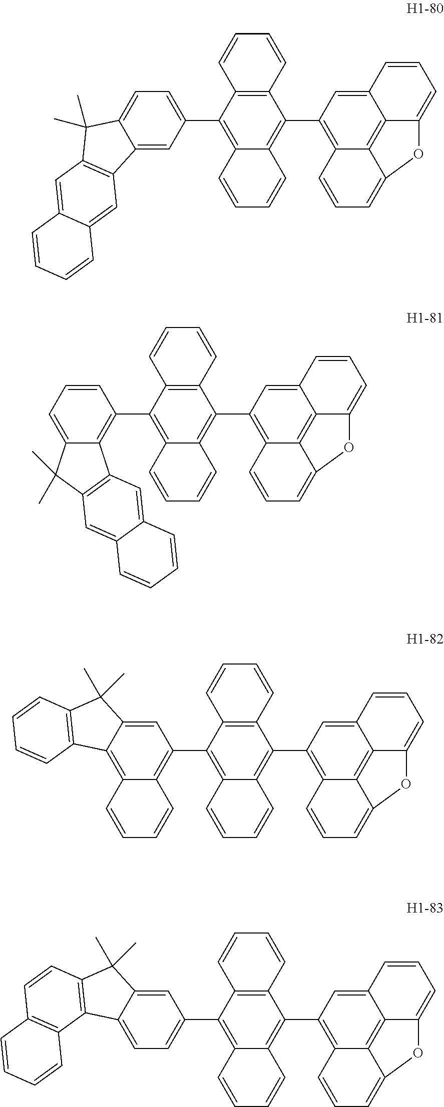 [SMILES: CC1(C)c2ccc(-c3c4ccccc4c(-c4cc5cccc6oc7cccc4c7c56)c4ccccc34)cc2-c2cc3ccccc3cc21 ; CC1(C)c2cc3ccccc3cc2-c2c(-c3c4ccccc4c(-c4cc5cccc6oc7cccc4c7c56)c4ccccc34)cccc21 ; CC1(C)c2ccccc2-c2c1cc(-c1c3ccccc3c(-c3cc4cccc5oc6cccc3c6c45)c3ccccc13)c1ccccc21 ; CC1(C)c2cc(-c3c4ccccc4c(-c4cc5cccc6oc7cccc4c7c56)c4ccccc34)ccc2-c2c1ccc1ccccc21] 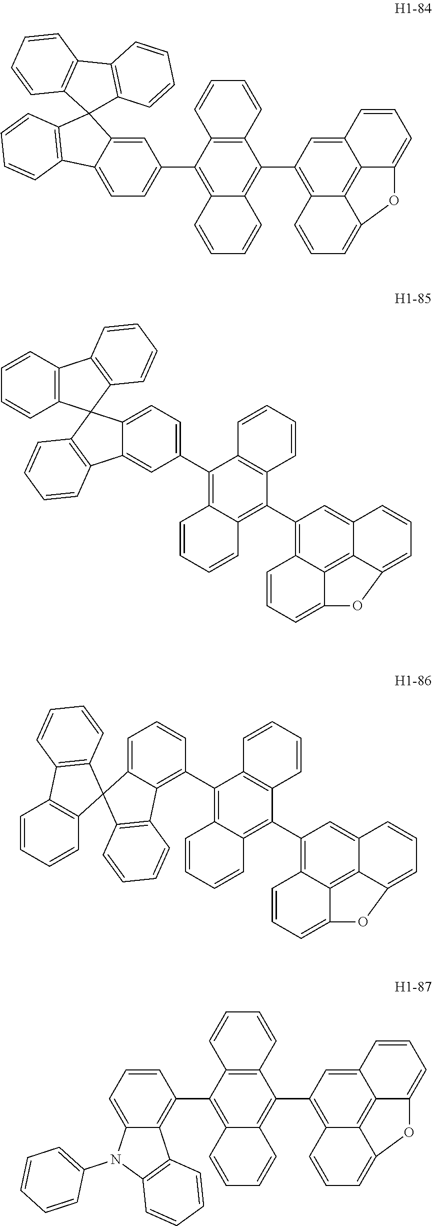 [SMILES: c1ccc2c(c1)-c1ccccc1C21c2ccccc2-c2ccc(-c3c4ccccc4c(-c4cc5cccc6oc7cccc4c7c56)c4ccccc34)cc21 ; c1ccc2c(c1)-c1ccccc1C21c2ccccc2-c2cc(-c3c4ccccc4c(-c4cc5cccc6oc7cccc4c7c56)c4ccccc34)ccc21 ; c1ccc2c(c1)-c1ccccc1C21c2ccccc2-c2c(-c3c4ccccc4c(-c4cc5cccc6oc7cccc4c7c56)c4ccccc34)cccc21 ; c1ccc(-n2c3ccccc3c3c(-c4c5ccccc5c(-c5cc6cccc7oc8cccc5c8c67)c5ccccc45)cccc32)cc1] 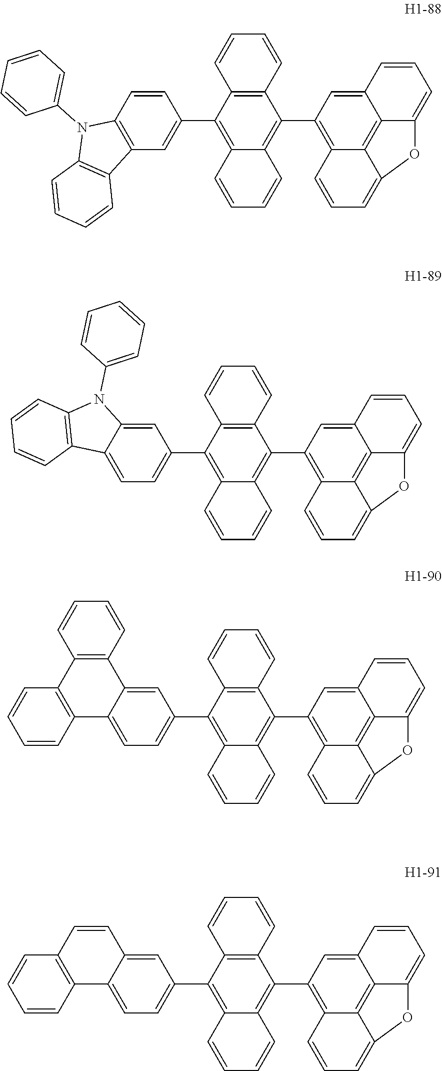 [SMILES: c1ccc(-n2c3ccccc3c3cc(-c4c5ccccc5c(-c5cc6cccc7oc8cccc5c8c67)c5ccccc45)ccc32)cc1 ; c1ccc(-n2c3ccccc3c3ccc(-c4c5ccccc5c(-c5cc6cccc7oc8cccc5c8c67)c5ccccc45)cc32)cc1 ; c1cc2cc(-c3c4ccccc4c(-c4ccc5c6ccccc6c6ccccc6c5c4)c4ccccc34)c3cccc4oc(c1)c2c43 ; c1ccc2c(c1)ccc1cc(-c3c4ccccc4c(-c4cc5cccc6oc7cccc4c7c56)c4ccccc34)ccc12] 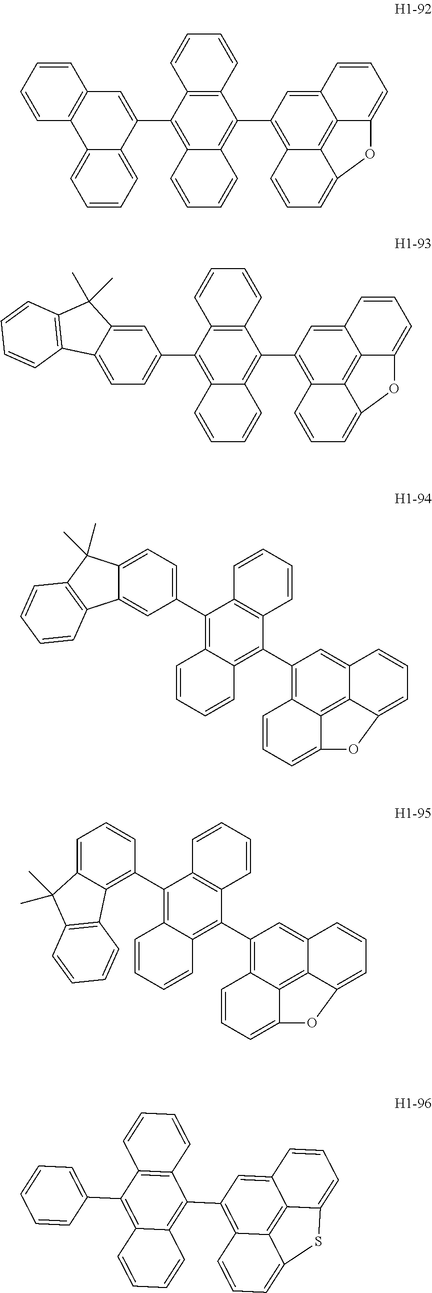 [SMILES: c1ccc2c(c1)cc(-c1c3ccccc3c(-c3cc4cccc5oc6cccc3c6c45)c3ccccc13)c1ccccc12 ; CC1(C)c2ccccc2-c2ccc(-c3c4ccccc4c(-c4cc5cccc6oc7cccc4c7c56)c4ccccc34)cc21 ; CC1(C)c2ccccc2-c2cc(-c3c4ccccc4c(-c4cc5cccc6oc7cccc4c7c56)c4ccccc34)ccc21 ; CC1(C)c2ccccc2-c2c(-c3c4ccccc4c(-c4cc5cccc6oc7cccc4c7c56)c4ccccc34)cccc21 ; c1ccc(-c2c3ccccc3c(-c3cc4cccc5sc6cccc3c6c45)c3ccccc23)cc1] 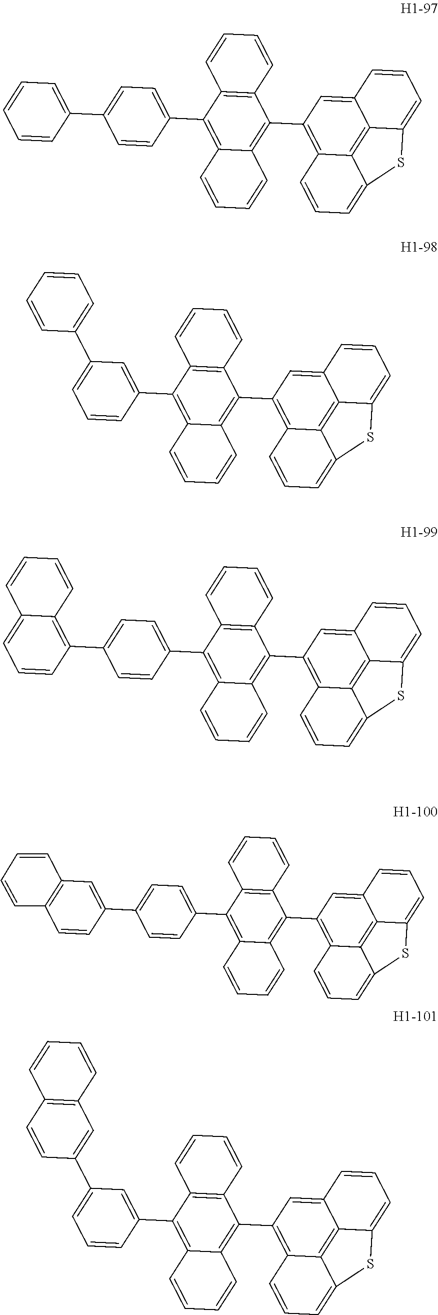 [SMILES: c1ccc(-c2ccc(-c3c4ccccc4c(-c4cc5cccc6sc7cccc4c7c56)c4ccccc34)cc2)cc1 ; c1ccc(-c2cccc(-c3c4ccccc4c(-c4cc5cccc6sc7cccc4c7c56)c4ccccc34)c2)cc1 ; c1ccc2c(-c3ccc(-c4c5ccccc5c(-c5cc6cccc7sc8cccc5c8c67)c5ccccc45)cc3)cccc2c1 ; c1ccc2cc(-c3ccc(-c4c5ccccc5c(-c5cc6cccc7sc8cccc5c8c67)c5ccccc45)cc3)ccc2c1 ; c1cc(-c2ccc3ccccc3c2)cc(-c2c3ccccc3c(-c3cc4cccc5sc6cccc3c6c45)c3ccccc23)c1] 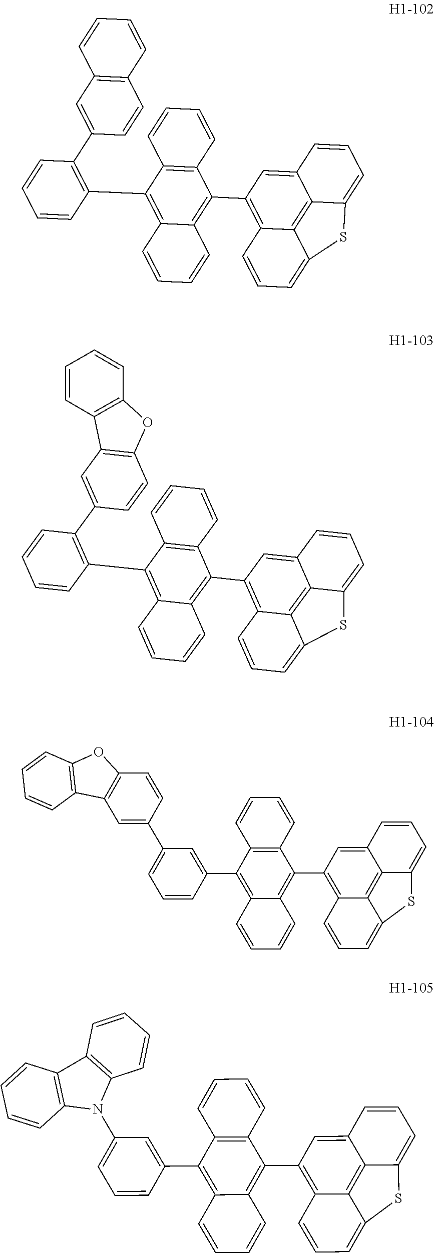 [SMILES: c1ccc(-c2c3ccccc3c(-c3cc4cccc5sc6cccc3c6c45)c3ccccc23)c(-c2ccc3ccccc3c2)c1 ; c1ccc(-c2c3ccccc3c(-c3cc4cccc5sc6cccc3c6c45)c3ccccc23)c(-c2ccc3oc4ccccc4c3c2)c1 ; c1cc(-c2ccc3oc4ccccc4c3c2)cc(-c2c3ccccc3c(-c3cc4cccc5sc6cccc3c6c45)c3ccccc23)c1 ; c1cc(-c2c3ccccc3c(-c3cc4cccc5sc6cccc3c6c45)c3ccccc23)cc(-n2c3ccccc3c3ccccc32)c1] 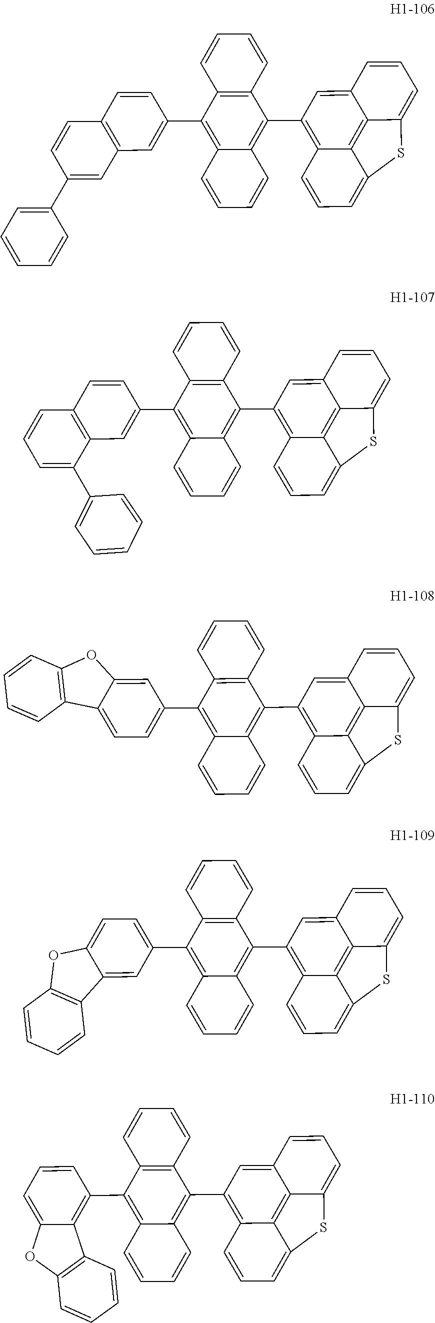 [SMILES: c1ccc(-c2ccc3ccc(-c4c5ccccc5c(-c5cc6cccc7sc8cccc5c8c67)c5ccccc45)cc3c2)cc1 ; c1ccc(-c2cccc3ccc(-c4c5ccccc5c(-c5cc6cccc7sc8cccc5c8c67)c5ccccc45)cc23)cc1 ; c1ccc2c(c1)oc1cc(-c3c4ccccc4c(-c4cc5cccc6sc7cccc4c7c56)c4ccccc34)ccc12 ; c1ccc2c(c1)oc1ccc(-c3c4ccccc4c(-c4cc5cccc6sc7cccc4c7c56)c4ccccc34)cc12 ; c1ccc2c(c1)oc1cccc(-c3c4ccccc4c(-c4cc5cccc6sc7cccc4c7c56)c4ccccc34)c12] 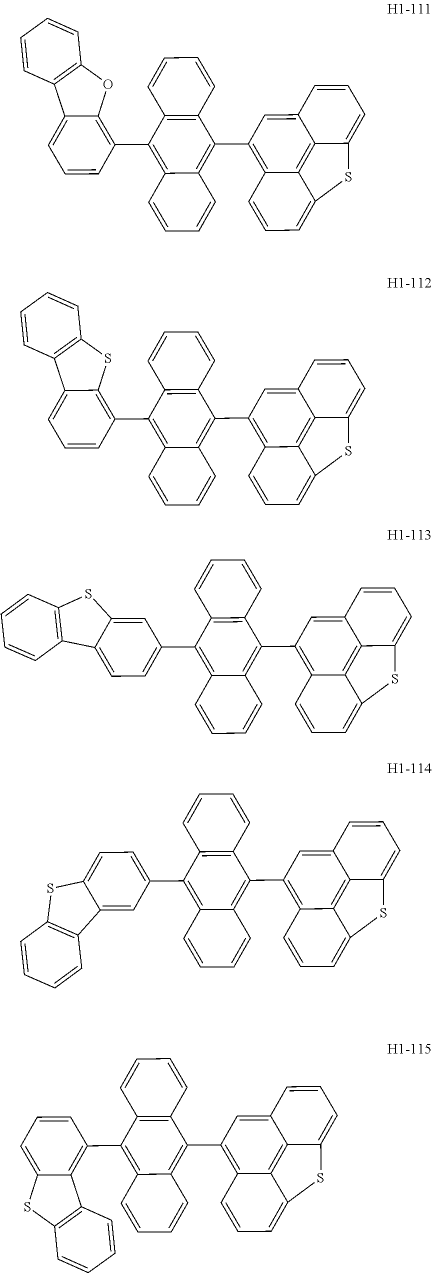 [SMILES: c1ccc2c(c1)oc1c(-c3c4ccccc4c(-c4cc5cccc6sc7cccc4c7c56)c4ccccc34)cccc12 ; c1ccc2c(c1)sc1c(-c3c4ccccc4c(-c4cc5cccc6sc7cccc4c7c56)c4ccccc34)cccc12 ; c1ccc2c(c1)sc1cc(-c3c4ccccc4c(-c4cc5cccc6sc7cccc4c7c56)c4ccccc34)ccc12 ; c1ccc2c(c1)sc1ccc(-c3c4ccccc4c(-c4cc5cccc6sc7cccc4c7c56)c4ccccc34)cc12 ; c1ccc2c(c1)sc1cccc(-c3c4ccccc4c(-c4cc5cccc6sc7cccc4c7c56)c4ccccc34)c12] 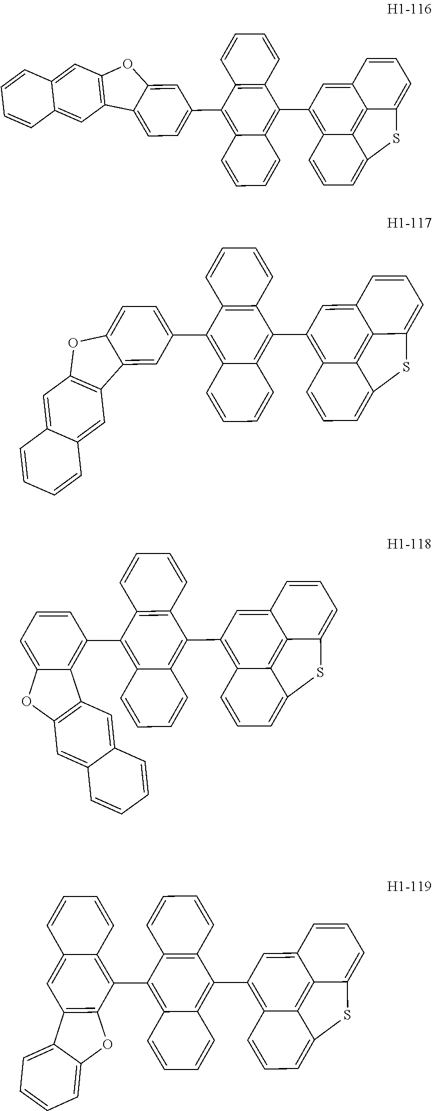 [SMILES: c1ccc2cc3c(cc2c1)oc1cc(-c2c4ccccc4c(-c4cc5cccc6sc7cccc4c7c56)c4ccccc24)ccc13 ; c1ccc2cc3c(cc2c1)oc1ccc(-c2c4ccccc4c(-c4cc5cccc6sc7cccc4c7c56)c4ccccc24)cc13 ; c1ccc2cc3c(cc2c1)oc1cccc(-c2c4ccccc4c(-c4cc5cccc6sc7cccc4c7c56)c4ccccc24)c13 ; c1ccc2c(-c3c4ccccc4c(-c4cc5cccc6sc7cccc4c7c56)c4ccccc34)c3oc4ccccc4c3cc2c1] 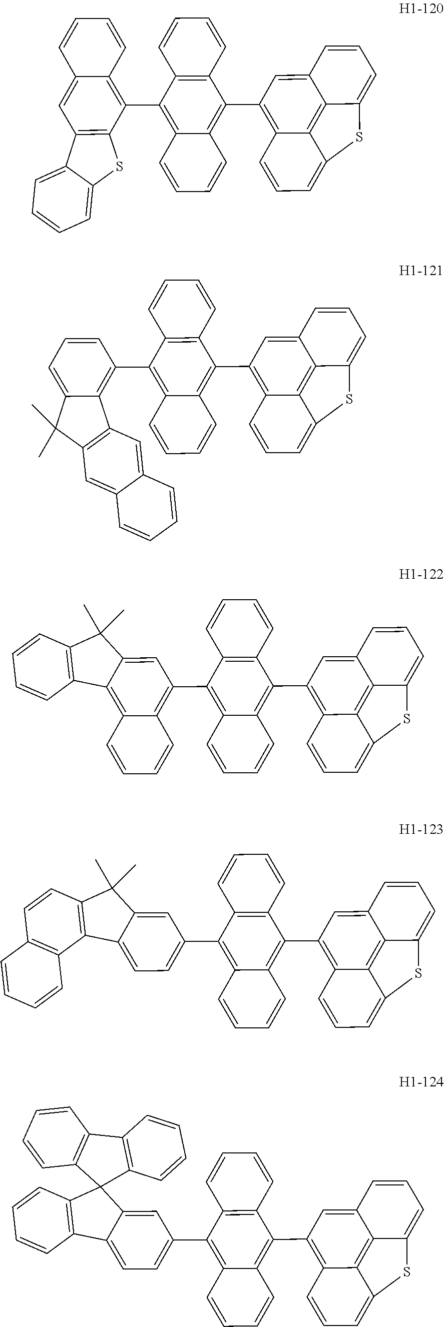 [SMILES: c1ccc2c(-c3c4ccccc4c(-c4cc5cccc6sc7cccc4c7c56)c4ccccc34)c3sc4ccccc4c3cc2c1 ; CC1(C)c2cc3ccccc3cc2-c2c(-c3c4ccccc4c(-c4cc5cccc6sc7cccc4c7c56)c4ccccc34)cccc21 ; CC1(C)c2ccccc2-c2c1cc(-c1c3ccccc3c(-c3cc4cccc5sc6cccc3c6c45)c3ccccc13)c1ccccc21 ; CC1(C)c2cc(-c3c4ccccc4c(-c4cc5cccc6sc7cccc4c7c56)c4ccccc34)ccc2-c2c1ccc1ccccc21 ; c1ccc2c(c1)-c1ccccc1C21c2ccccc2-c2ccc(-c3c4ccccc4c(-c4cc5cccc6sc7cccc4c7c56)c4ccccc34)cc21] 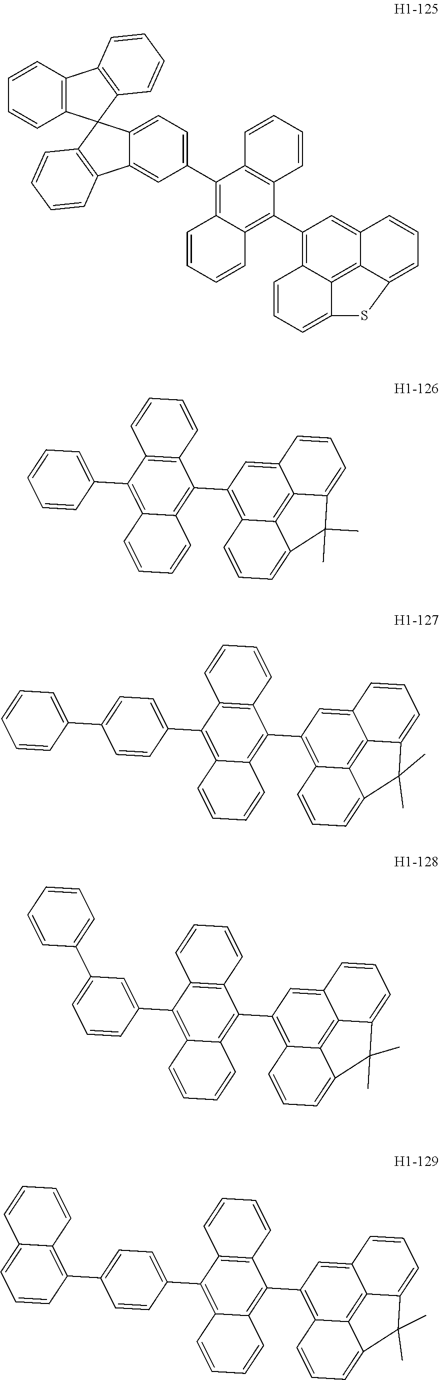 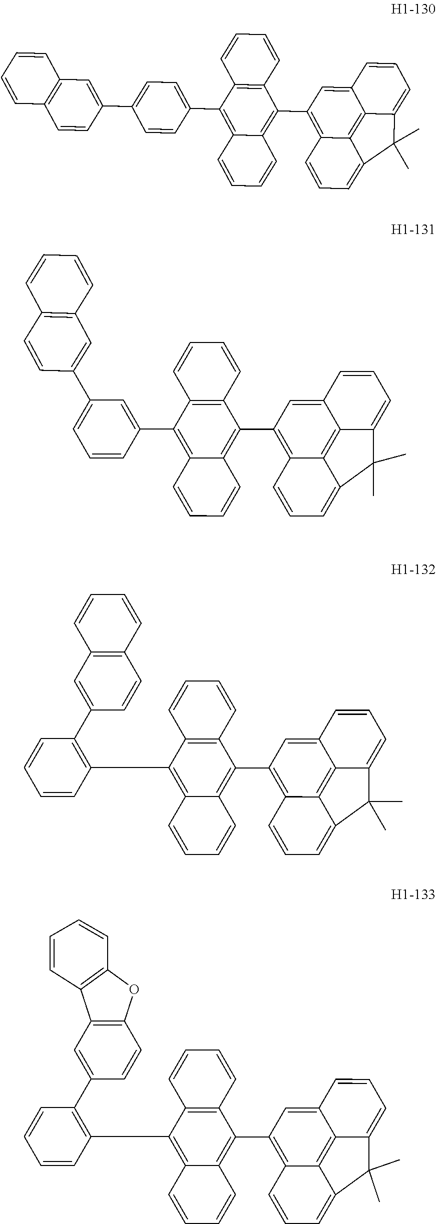 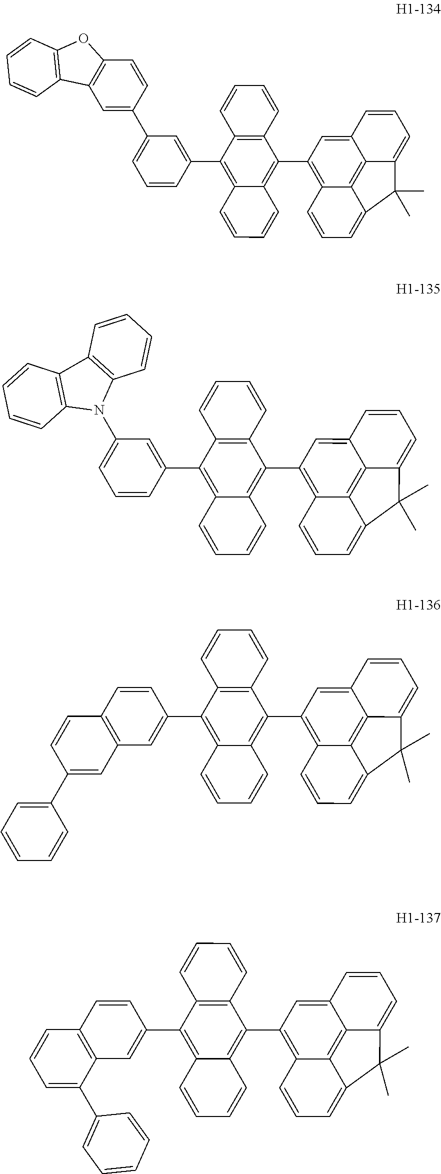 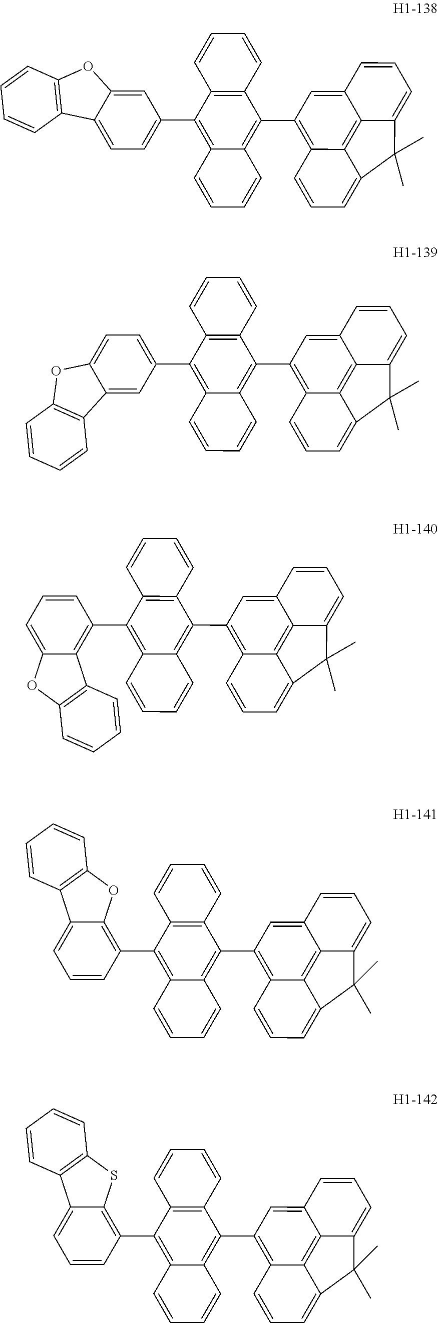 [SMILES: CC1(C)c2cccc3cc(-c4c5ccccc5c(-c5ccc6c(c5)oc5ccccc56)c5ccccc45)c4cccc1c4c23 ; CC1(C)c2cccc3cc(-c4c5ccccc5c(-c5ccc6oc7ccccc7c6c5)c5ccccc45)c4cccc1c4c23 ; CC1(C)c2cccc3cc(-c4c5ccccc5c(-c5cccc6oc7ccccc7c56)c5ccccc45)c4cccc1c4c23 ; CC1(C)c2cccc3cc(-c4c5ccccc5c(-c5cccc6c5oc5ccccc56)c5ccccc45)c4cccc1c4c23 ; CC1(C)c2cccc3cc(-c4c5ccccc5c(-c5cccc6c5sc5ccccc56)c5ccccc45)c4cccc1c4c23] 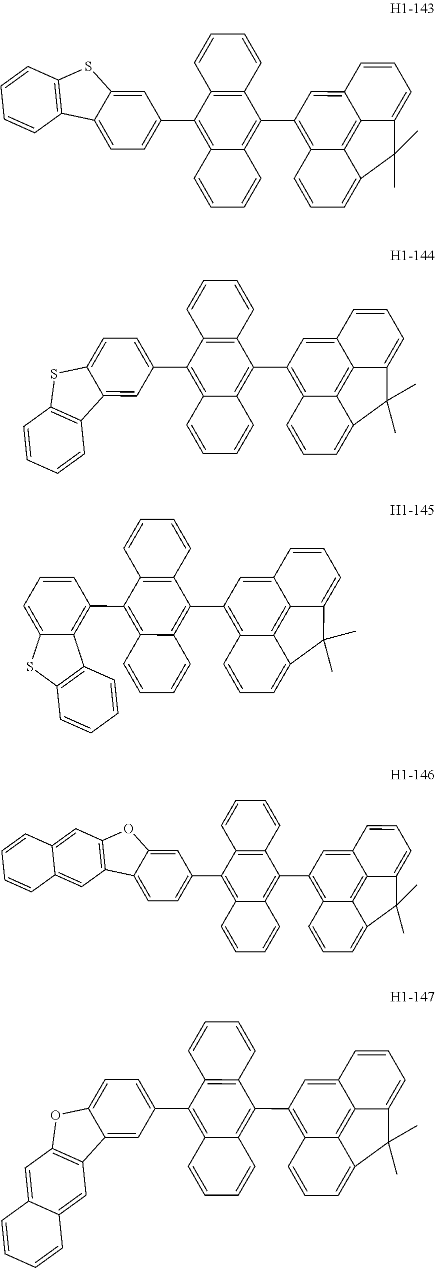 [SMILES: CC1(C)c2cccc3cc(-c4c5ccccc5c(-c5ccc6c(c5)sc5ccccc56)c5ccccc45)c4cccc1c4c23 ; CC1(C)c2cccc3cc(-c4c5ccccc5c(-c5ccc6sc7ccccc7c6c5)c5ccccc45)c4cccc1c4c23 ; CC1(C)c2cccc3cc(-c4c5ccccc5c(-c5cccc6sc7ccccc7c56)c5ccccc45)c4cccc1c4c23 ; CC1(C)c2cccc3cc(-c4c5ccccc5c(-c5ccc6c(c5)oc5cc7ccccc7cc56)c5ccccc45)c4cccc1c4c23 ; CC1(C)c2cccc3cc(-c4c5ccccc5c(-c5ccc6oc7cc8ccccc8cc7c6c5)c5ccccc45)c4cccc1c4c23] 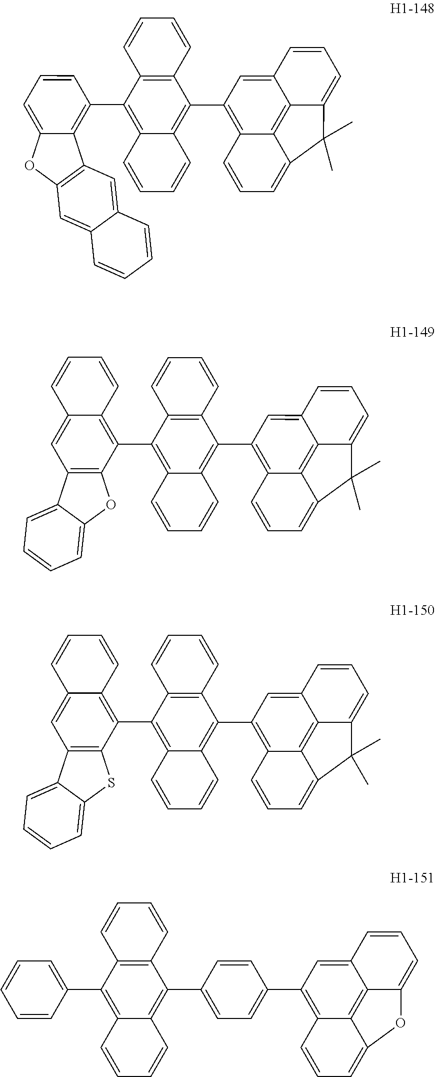 [SMILES: CC1(C)c2cccc3cc(-c4c5ccccc5c(-c5cccc6oc7cc8ccccc8cc7c56)c5ccccc45)c4cccc1c4c23 ; CC1(C)c2cccc3cc(-c4c5ccccc5c(-c5c6ccccc6cc6c5oc5ccccc56)c5ccccc45)c4cccc1c4c23 ; CC1(C)c2cccc3cc(-c4c5ccccc5c(-c5c6ccccc6cc6c5sc5ccccc56)c5ccccc45)c4cccc1c4c23 ; c1ccc(-c2c3ccccc3c(-c3ccc(-c4cc5cccc6oc7cccc4c7c56)cc3)c3ccccc23)cc1] 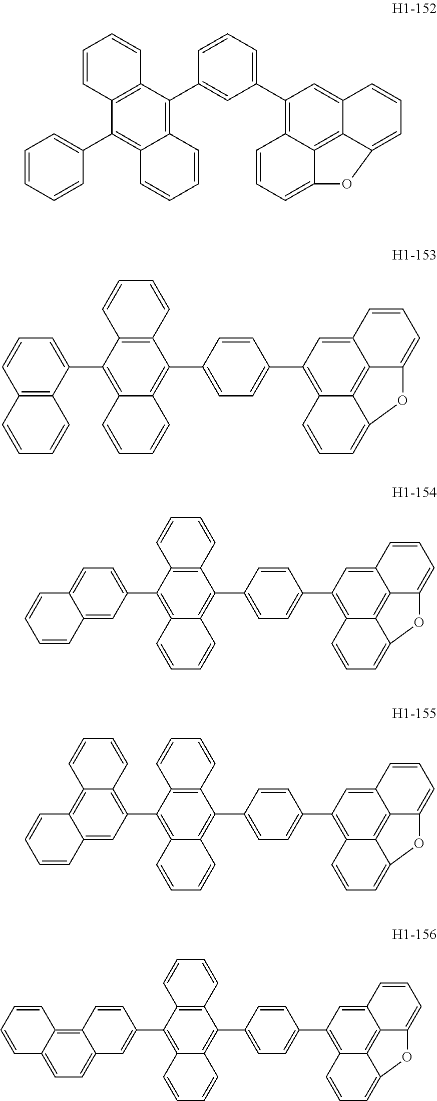 [SMILES: c1ccc(-c2c3ccccc3c(-c3cccc(-c4cc5cccc6oc7cccc4c7c56)c3)c3ccccc23)cc1 ; c1ccc2c(-c3c4ccccc4c(-c4ccc(-c5cc6cccc7oc8cccc5c8c67)cc4)c4ccccc34)cccc2c1 ; c1ccc2cc(-c3c4ccccc4c(-c4ccc(-c5cc6cccc7oc8cccc5c8c67)cc4)c4ccccc34)ccc2c1 ; c1ccc2c(c1)cc(-c1c3ccccc3c(-c3ccc(-c4cc5cccc6oc7cccc4c7c56)cc3)c3ccccc13)c1ccccc12 ; c1ccc2c(c1)ccc1cc(-c3c4ccccc4c(-c4ccc(-c5cc6cccc7oc8cccc5c8c67)cc4)c4ccccc34)ccc12] 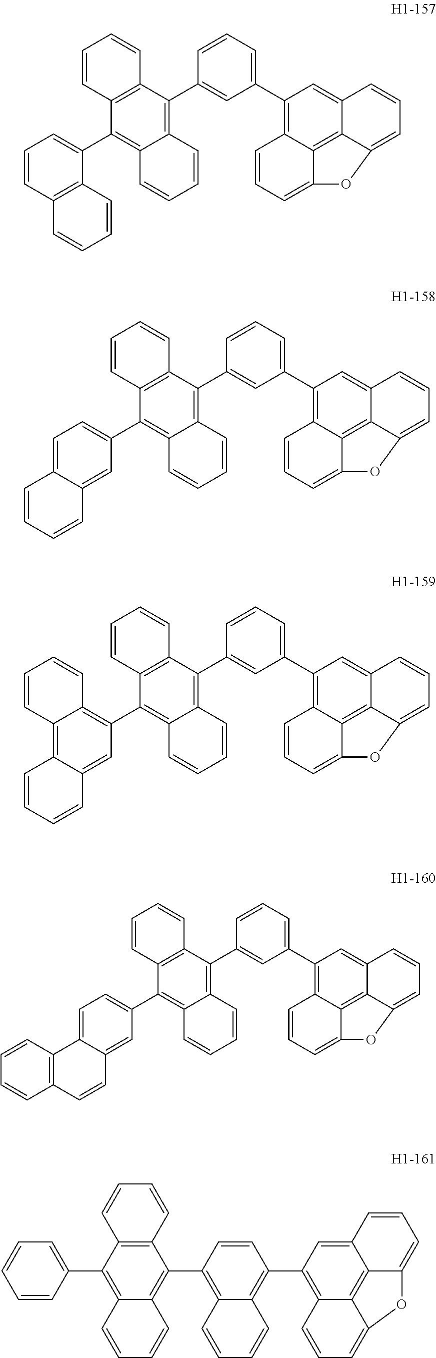 [SMILES: c1cc(-c2c3ccccc3c(-c3cccc4ccccc34)c3ccccc23)cc(-c2cc3cccc4oc5cccc2c5c34)c1 ; c1cc(-c2c3ccccc3c(-c3ccc4ccccc4c3)c3ccccc23)cc(-c2cc3cccc4oc5cccc2c5c34)c1 ; c1cc(-c2c3ccccc3c(-c3cc4ccccc4c4ccccc34)c3ccccc23)cc(-c2cc3cccc4oc5cccc2c5c34)c1 ; c1cc(-c2c3ccccc3c(-c3ccc4c(ccc5ccccc54)c3)c3ccccc23)cc(-c2cc3cccc4oc5cccc2c5c34)c1 ; c1ccc(-c2c3ccccc3c(-c3ccc(-c4cc5cccc6oc7cccc4c7c56)c4ccccc34)c3ccccc23)cc1] 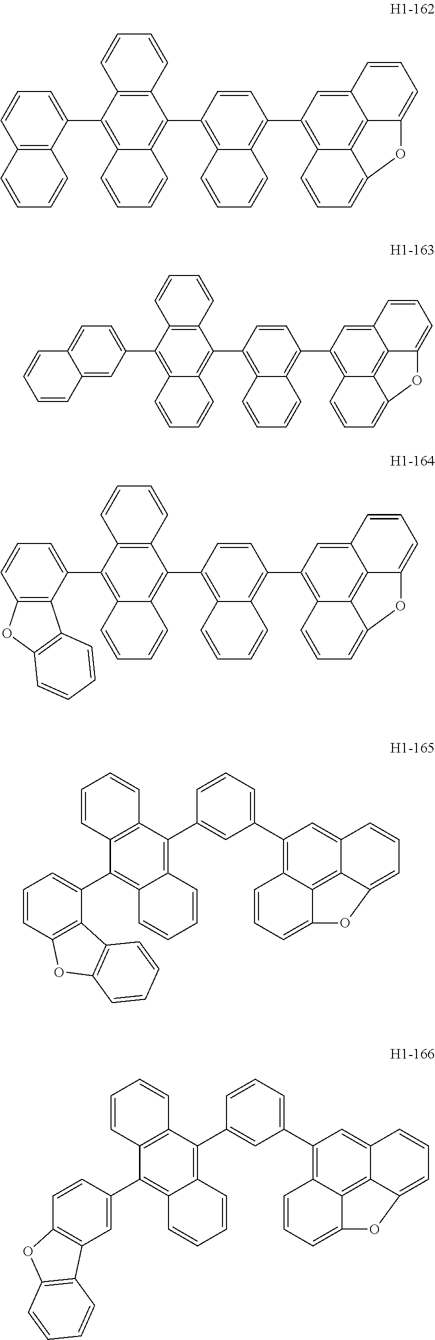 [SMILES: c1ccc2c(-c3c4ccccc4c(-c4ccc(-c5cc6cccc7oc8cccc5c8c67)c5ccccc45)c4ccccc34)cccc2c1 ; c1ccc2cc(-c3c4ccccc4c(-c4ccc(-c5cc6cccc7oc8cccc5c8c67)c5ccccc45)c4ccccc34)ccc2c1 ; c1ccc2c(c1)oc1cccc(-c3c4ccccc4c(-c4ccc(-c5cc6cccc7oc8cccc5c8c67)c5ccccc45)c4ccccc34)c12 ; c1cc(-c2c3ccccc3c(-c3cccc4oc5ccccc5c34)c3ccccc23)cc(-c2cc3cccc4oc5cccc2c5c34)c1 ; c1cc(-c2c3ccccc3c(-c3ccc4oc5ccccc5c4c3)c3ccccc23)cc(-c2cc3cccc4oc5cccc2c5c34)c1] 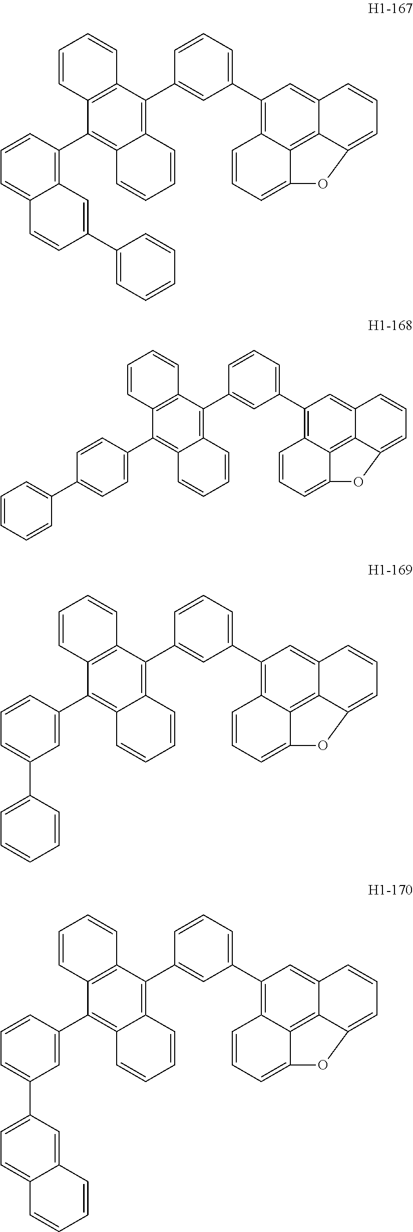 [SMILES: c1ccc(-c2ccc3cccc(-c4c5ccccc5c(-c5cccc(-c6cc7cccc8oc9cccc6c9c78)c5)c5ccccc45)c3c2)cc1 ; c1ccc(-c2ccc(-c3c4ccccc4c(-c4cccc(-c5cc6cccc7oc8cccc5c8c67)c4)c4ccccc34)cc2)cc1 ; c1ccc(-c2cccc(-c3c4ccccc4c(-c4cccc(-c5cc6cccc7oc8cccc5c8c67)c4)c4ccccc34)c2)cc1 ; c1cc(-c2ccc3ccccc3c2)cc(-c2c3ccccc3c(-c3cccc(-c4cc5cccc6oc7cccc4c7c56)c3)c3ccccc23)c1] 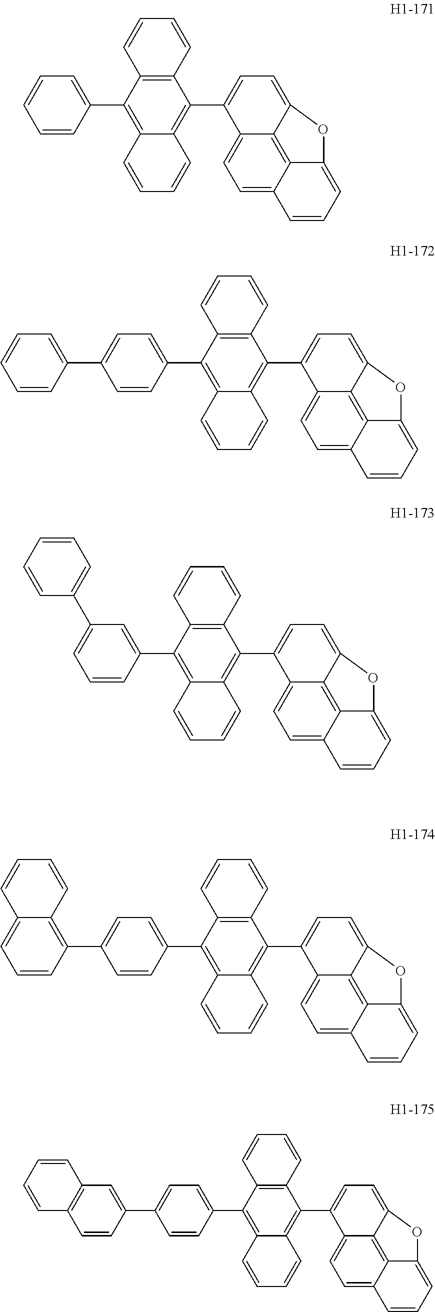 [SMILES: c1ccc(-c2c3ccccc3c(-c3ccc4oc5cccc6ccc3c4c65)c3ccccc23)cc1 ; c1ccc(-c2ccc(-c3c4ccccc4c(-c4ccc5oc6cccc7ccc4c5c76)c4ccccc34)cc2)cc1 ; c1ccc(-c2cccc(-c3c4ccccc4c(-c4ccc5oc6cccc7ccc4c5c76)c4ccccc34)c2)cc1 ; c1ccc2c(-c3ccc(-c4c5ccccc5c(-c5ccc6oc7cccc8ccc5c6c87)c5ccccc45)cc3)cccc2c1 ; c1ccc2cc(-c3ccc(-c4c5ccccc5c(-c5ccc6oc7cccc8ccc5c6c87)c5ccccc45)cc3)ccc2c1] 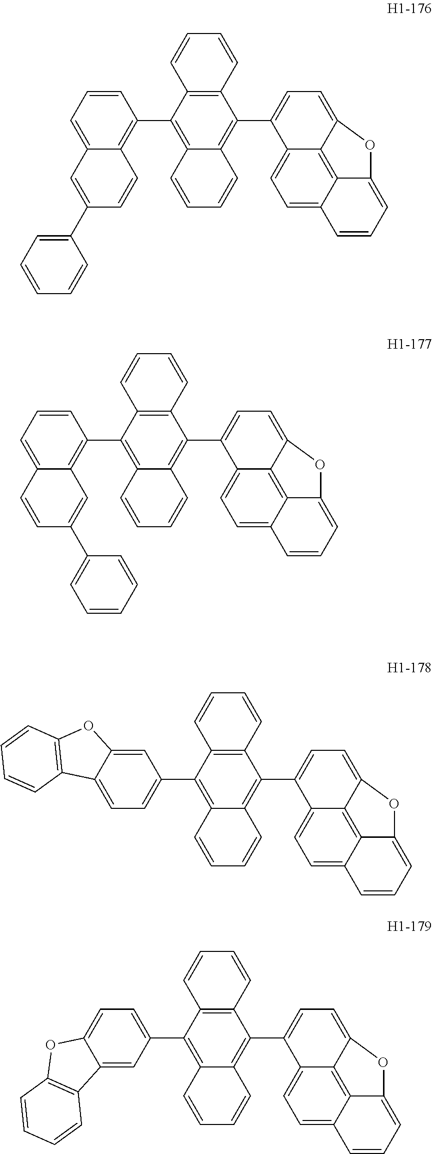 [SMILES: c1ccc(-c2ccc3c(-c4c5ccccc5c(-c5ccc6oc7cccc8ccc5c6c87)c5ccccc45)cccc3c2)cc1 ; c1ccc(-c2ccc3cccc(-c4c5ccccc5c(-c5ccc6oc7cccc8ccc5c6c87)c5ccccc45)c3c2)cc1 ; c1ccc2c(c1)oc1cc(-c3c4ccccc4c(-c4ccc5oc6cccc7ccc4c5c76)c4ccccc34)ccc12 ; c1ccc2c(c1)oc1ccc(-c3c4ccccc4c(-c4ccc5oc6cccc7ccc4c5c76)c4ccccc34)cc12] 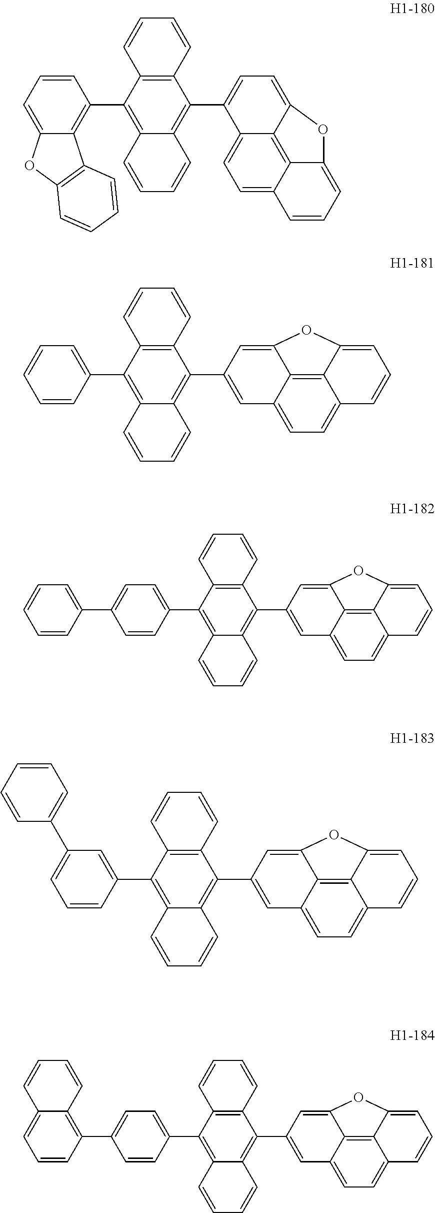 [SMILES: c1ccc2c(c1)oc1cccc(-c3c4ccccc4c(-c4ccc5oc6cccc7ccc4c5c76)c4ccccc34)c12 ; c1ccc(-c2c3ccccc3c(-c3cc4ccc5cccc6oc(c3)c4c56)c3ccccc23)cc1 ; c1ccc(-c2ccc(-c3c4ccccc4c(-c4cc5ccc6cccc7oc(c4)c5c67)c4ccccc34)cc2)cc1 ; c1ccc(-c2cccc(-c3c4ccccc4c(-c4cc5ccc6cccc7oc(c4)c5c67)c4ccccc34)c2)cc1 ; c1ccc2c(-c3ccc(-c4c5ccccc5c(-c5cc6ccc7cccc8oc(c5)c6c78)c5ccccc45)cc3)cccc2c1] 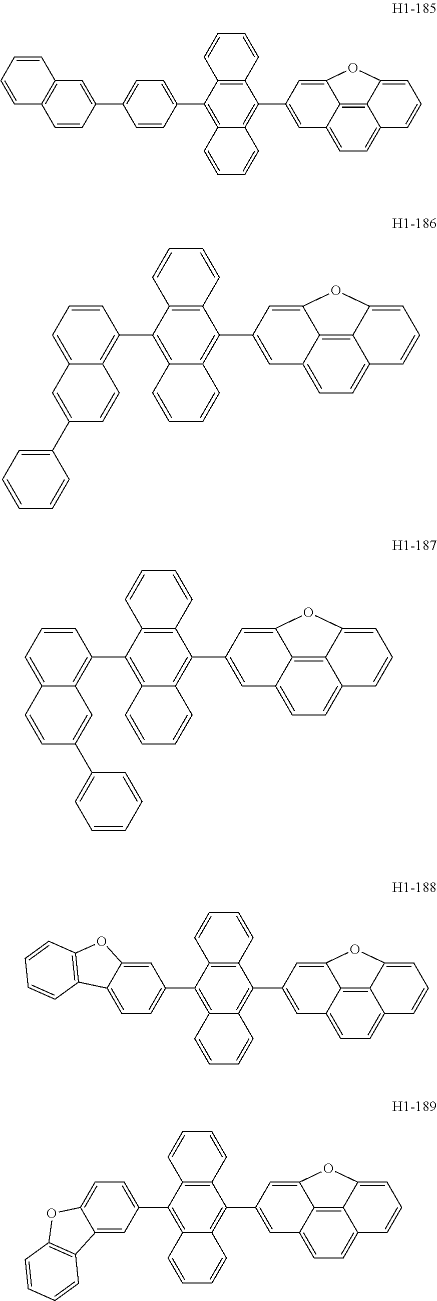 [SMILES: c1ccc2cc(-c3ccc(-c4c5ccccc5c(-c5cc6ccc7cccc8oc(c5)c6c78)c5ccccc45)cc3)ccc2c1 ; c1ccc(-c2ccc3c(-c4c5ccccc5c(-c5cc6ccc7cccc8oc(c5)c6c78)c5ccccc45)cccc3c2)cc1 ; c1ccc(-c2ccc3cccc(-c4c5ccccc5c(-c5cc6ccc7cccc8oc(c5)c6c78)c5ccccc45)c3c2)cc1 ; c1ccc2c(c1)oc1cc(-c3c4ccccc4c(-c4cc5ccc6cccc7oc(c4)c5c67)c4ccccc34)ccc12 ; c1ccc2c(c1)oc1ccc(-c3c4ccccc4c(-c4cc5ccc6cccc7oc(c4)c5c67)c4ccccc34)cc12] 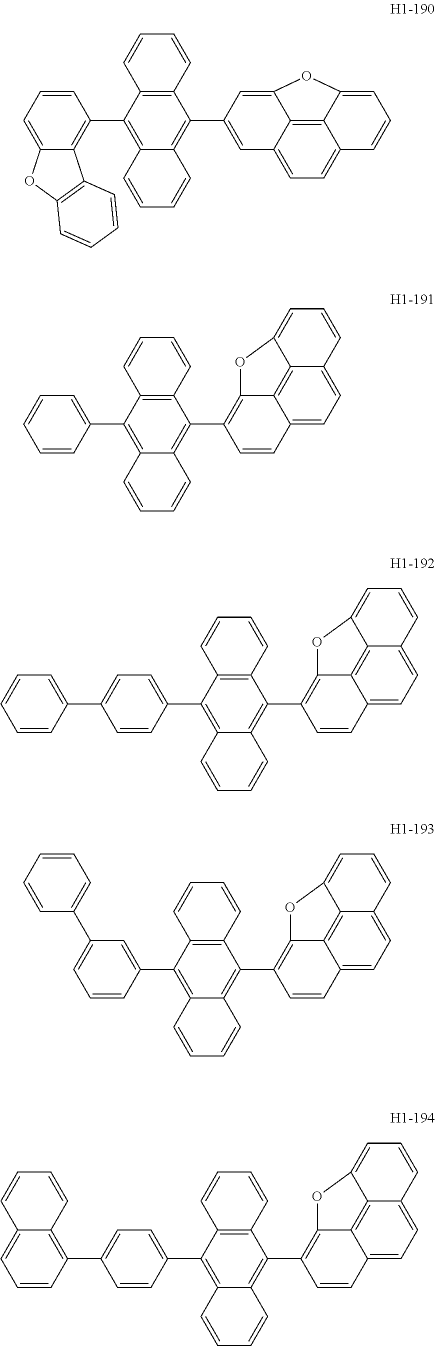 [SMILES: c1ccc2c(c1)oc1cccc(-c3c4ccccc4c(-c4cc5ccc6cccc7oc(c4)c5c67)c4ccccc34)c12 ; c1ccc(-c2c3ccccc3c(-c3ccc4ccc5cccc6oc3c4c56)c3ccccc23)cc1 ; c1ccc(-c2ccc(-c3c4ccccc4c(-c4ccc5ccc6cccc7oc4c5c67)c4ccccc34)cc2)cc1 ; c1ccc(-c2cccc(-c3c4ccccc4c(-c4ccc5ccc6cccc7oc4c5c67)c4ccccc34)c2)cc1 ; c1ccc2c(-c3ccc(-c4c5ccccc5c(-c5ccc6ccc7cccc8oc5c6c78)c5ccccc45)cc3)cccc2c1] 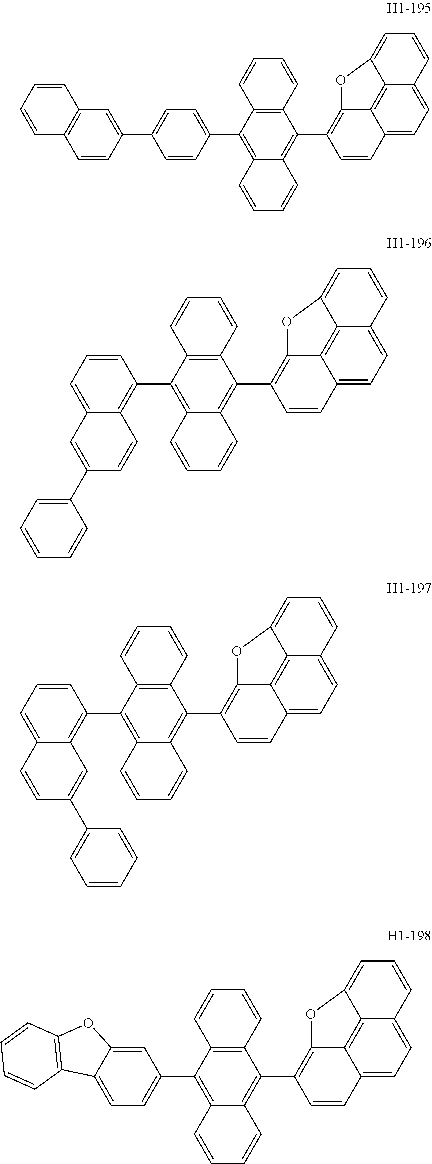 [SMILES: c1ccc2cc(-c3ccc(-c4c5ccccc5c(-c5ccc6ccc7cccc8oc5c6c78)c5ccccc45)cc3)ccc2c1 ; c1ccc(-c2ccc3c(-c4c5ccccc5c(-c5ccc6ccc7cccc8oc5c6c78)c5ccccc45)cccc3c2)cc1 ; c1ccc(-c2ccc3cccc(-c4c5ccccc5c(-c5ccc6ccc7cccc8oc5c6c78)c5ccccc45)c3c2)cc1 ; c1ccc2c(c1)oc1cc(-c3c4ccccc4c(-c4ccc5ccc6cccc7oc4c5c67)c4ccccc34)ccc12] 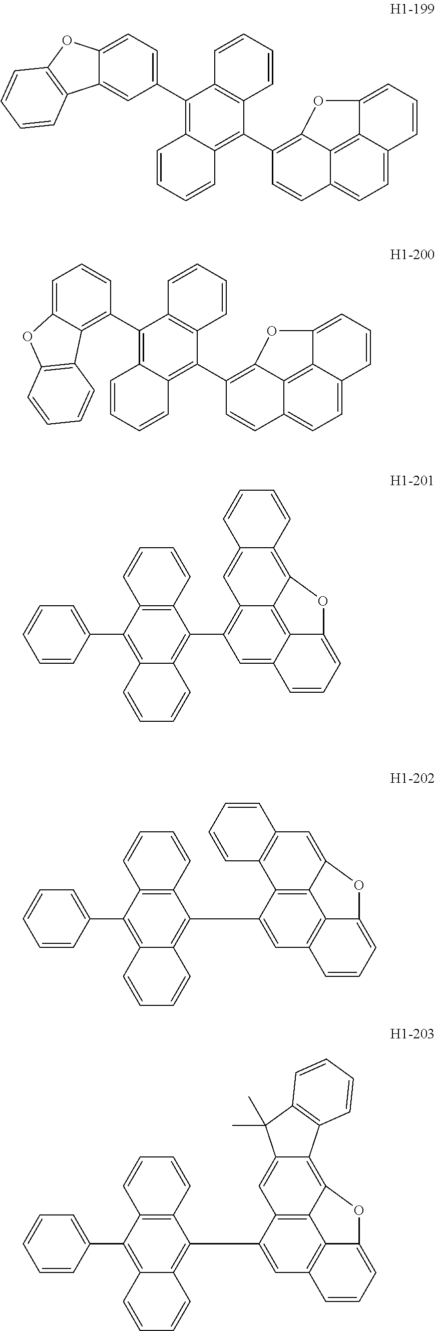 [SMILES: c1ccc2c(c1)oc1ccc(-c3c4ccccc4c(-c4ccc5ccc6cccc7oc4c5c67)c4ccccc34)cc12 ; c1ccc2c(c1)oc1cccc(-c3c4ccccc4c(-c4ccc5ccc6cccc7oc4c5c67)c4ccccc34)c12 ; c1ccc(-c2c3ccccc3c(-c3cc4cccc5oc6c7ccccc7cc3c6c45)c3ccccc23)cc1 ; c1ccc(-c2c3ccccc3c(-c3cc4cccc5oc6cc7ccccc7c3c6c45)c3ccccc23)cc1 ; CC1(C)c2ccccc2-c2c1cc1c(-c3c4ccccc4c(-c4ccccc4)c4ccccc34)cc3cccc4oc2c1c34] 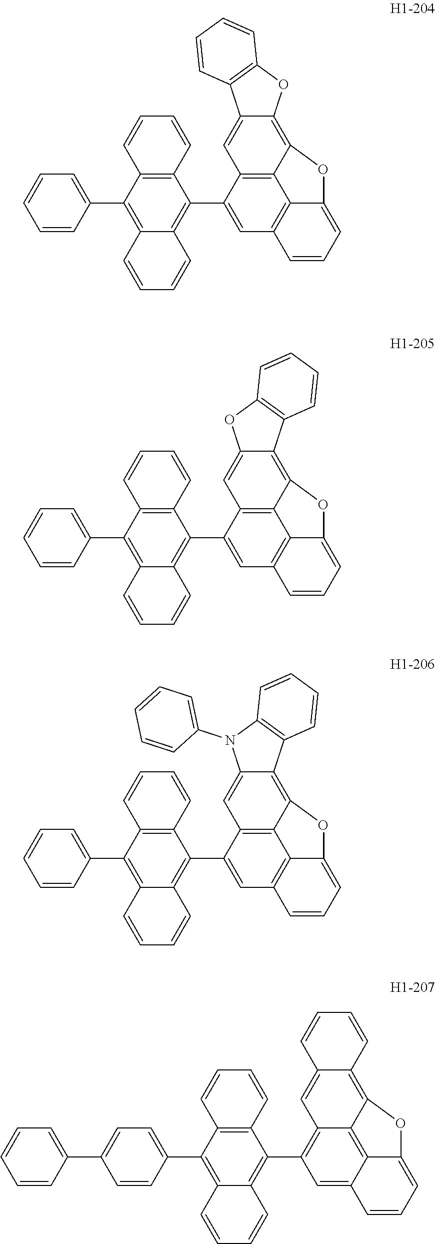 [SMILES: c1ccc(-c2c3ccccc3c(-c3cc4cccc5oc6c7oc8ccccc8c7cc3c6c45)c3ccccc23)cc1 ; c1ccc(-c2c3ccccc3c(-c3cc4cccc5oc6c7c(cc3c6c45)oc3ccccc37)c3ccccc23)cc1 ; c1ccc(-c2c3ccccc3c(-c3cc4cccc5oc6c(c3cc3c6c6ccccc6n3-c3ccccc3)c45)c3ccccc23)cc1 ; c1ccc(-c2ccc(-c3c4ccccc4c(-c4cc5cccc6oc7c8ccccc8cc4c7c56)c4ccccc34)cc2)cc1] 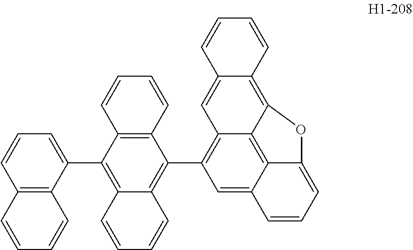 [SMILES: c1ccc2c(-c3c4ccccc4c(-c4cc5cccc6oc7c8ccccc8cc4c7c56)c4ccccc34)cccc2c1] 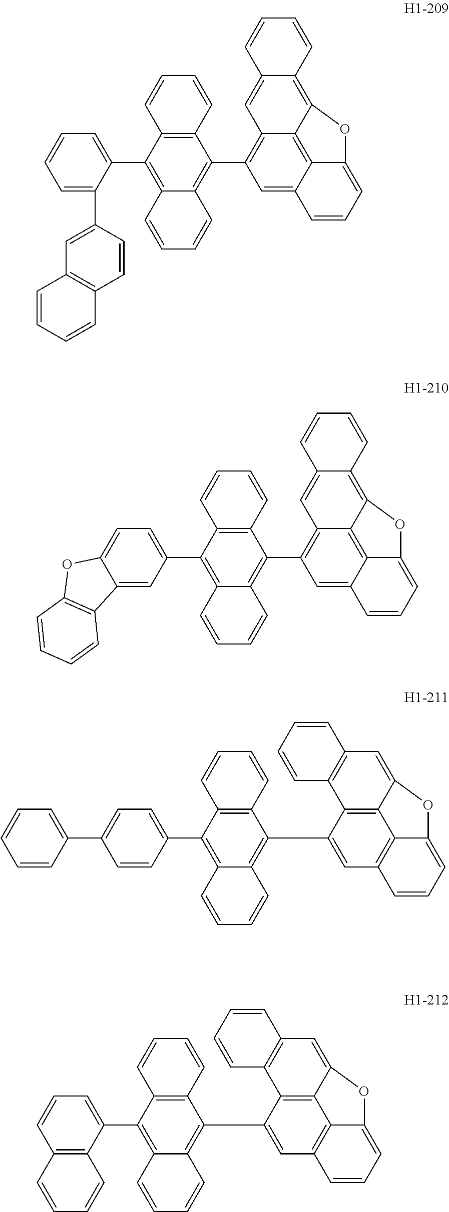 [SMILES: c1ccc(-c2c3ccccc3c(-c3cc4cccc5oc6c7ccccc7cc3c6c45)c3ccccc23)c(-c2ccc3ccccc3c2)c1 ; c1ccc2c(c1)cc1c(-c3c4ccccc4c(-c4ccc5oc6ccccc6c5c4)c4ccccc34)cc3cccc4oc2c1c34 ; c1ccc(-c2ccc(-c3c4ccccc4c(-c4cc5cccc6oc7cc8ccccc8c4c7c56)c4ccccc34)cc2)cc1 ; c1ccc2c(-c3c4ccccc4c(-c4cc5cccc6oc7cc8ccccc8c4c7c56)c4ccccc34)cccc2c1] 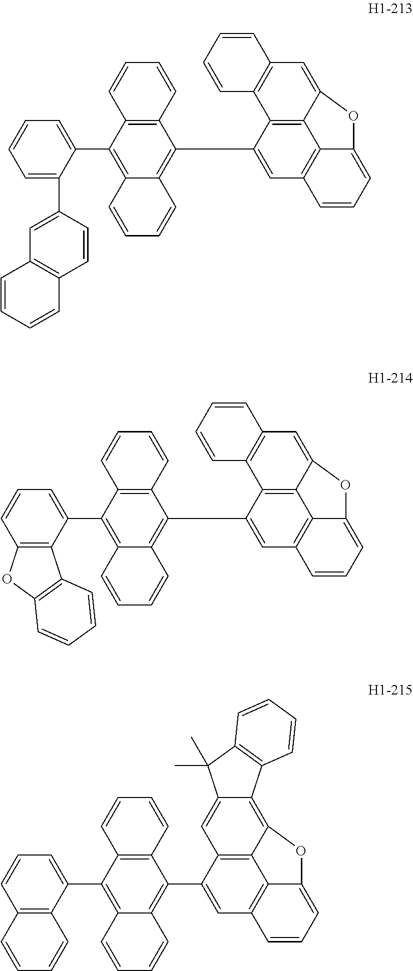 [SMILES: c1ccc(-c2c3ccccc3c(-c3cc4cccc5oc6cc7ccccc7c3c6c45)c3ccccc23)c(-c2ccc3ccccc3c2)c1 ; c1ccc2c(c1)cc1oc3cccc4cc(-c5c6ccccc6c(-c6cccc7oc8ccccc8c67)c6ccccc56)c2c1c43 ; CC1(C)c2ccccc2-c2c1cc1c(-c3c4ccccc4c(-c4cccc5ccccc45)c4ccccc34)cc3cccc4oc2c1c34] 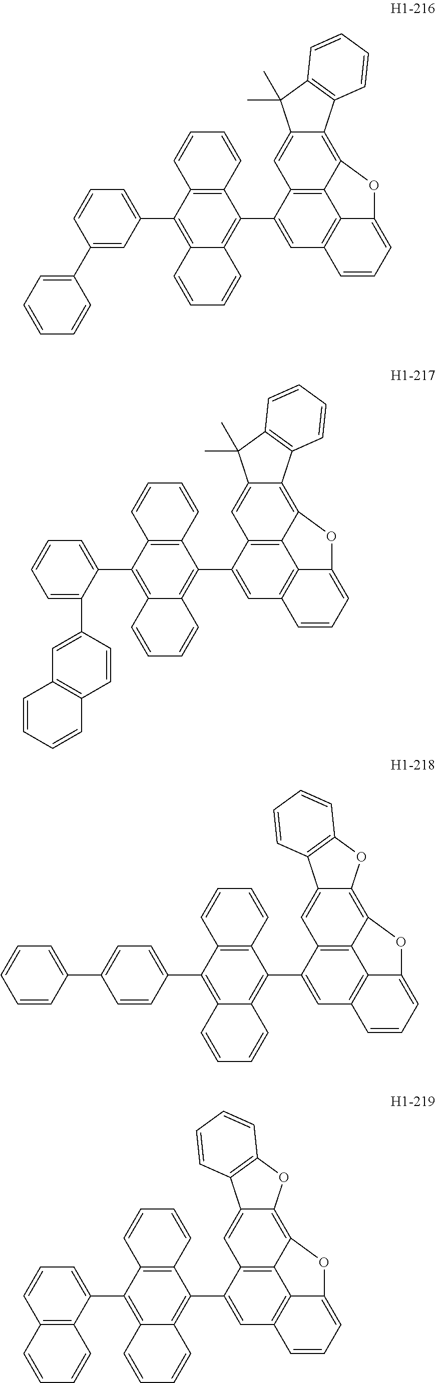 [SMILES: CC1(C)c2ccccc2-c2c1cc1c(-c3c4ccccc4c(-c4cccc(-c5ccccc5)c4)c4ccccc34)cc3cccc4oc2c1c34 ; CC1(C)c2ccccc2-c2c1cc1c(-c3c4ccccc4c(-c4ccccc4-c4ccc5ccccc5c4)c4ccccc34)cc3cccc4oc2c1c34 ; c1ccc(-c2ccc(-c3c4ccccc4c(-c4cc5cccc6oc7c8oc9ccccc9c8cc4c7c56)c4ccccc34)cc2)cc1 ; c1ccc2c(-c3c4ccccc4c(-c4cc5cccc6oc7c8oc9ccccc9c8cc4c7c56)c4ccccc34)cccc2c1] 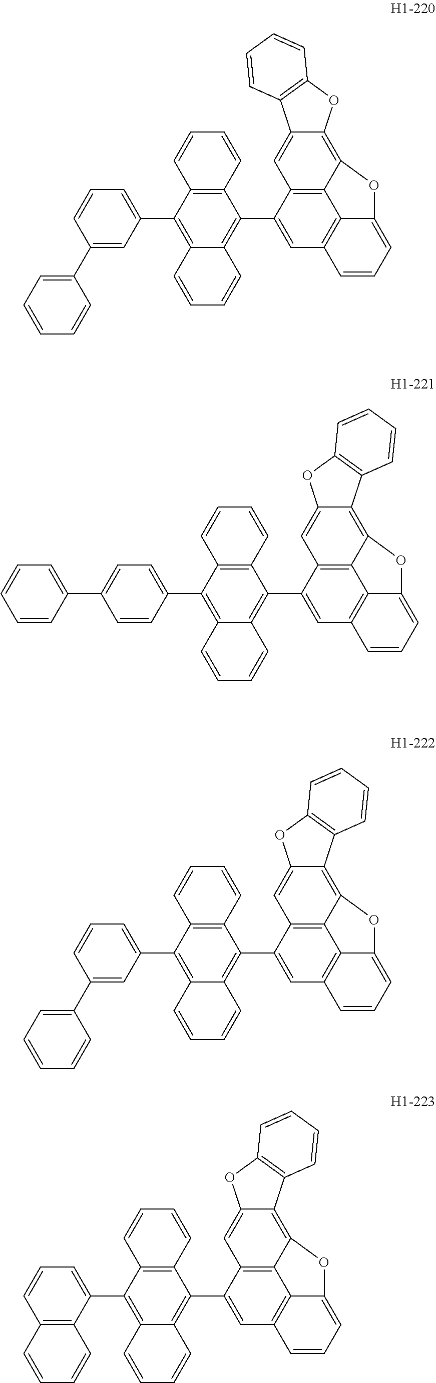 [SMILES: c1ccc(-c2cccc(-c3c4ccccc4c(-c4cc5cccc6oc7c8oc9ccccc9c8cc4c7c56)c4ccccc34)c2)cc1 ; c1ccc(-c2ccc(-c3c4ccccc4c(-c4cc5cccc6oc7c8c(cc4c7c56)oc4ccccc48)c4ccccc34)cc2)cc1 ; c1ccc(-c2cccc(-c3c4ccccc4c(-c4cc5cccc6oc7c8c(cc4c7c56)oc4ccccc48)c4ccccc34)c2)cc1 ; c1ccc2c(-c3c4ccccc4c(-c4cc5cccc6oc7c8c(cc4c7c56)oc4ccccc48)c4ccccc34)cccc2c1] 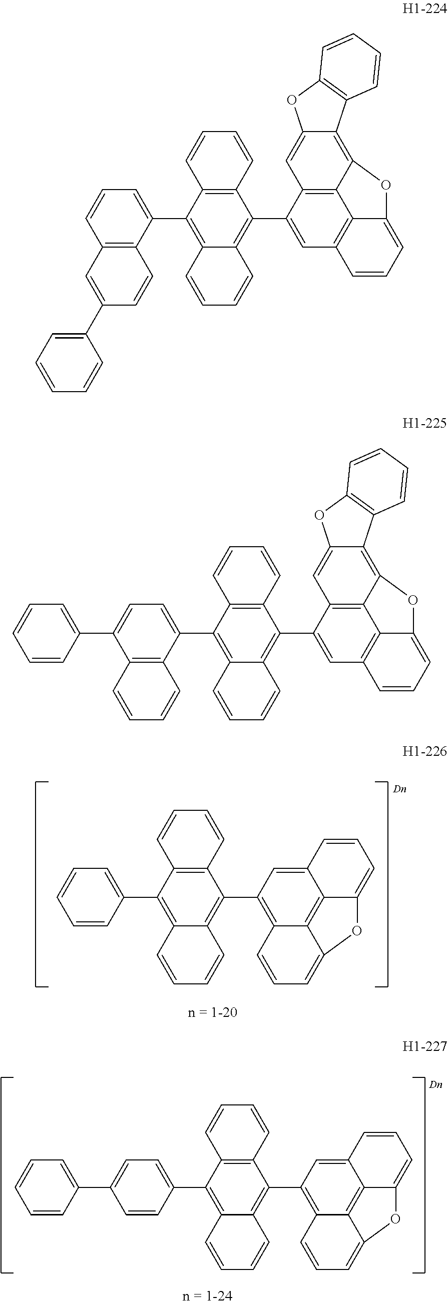 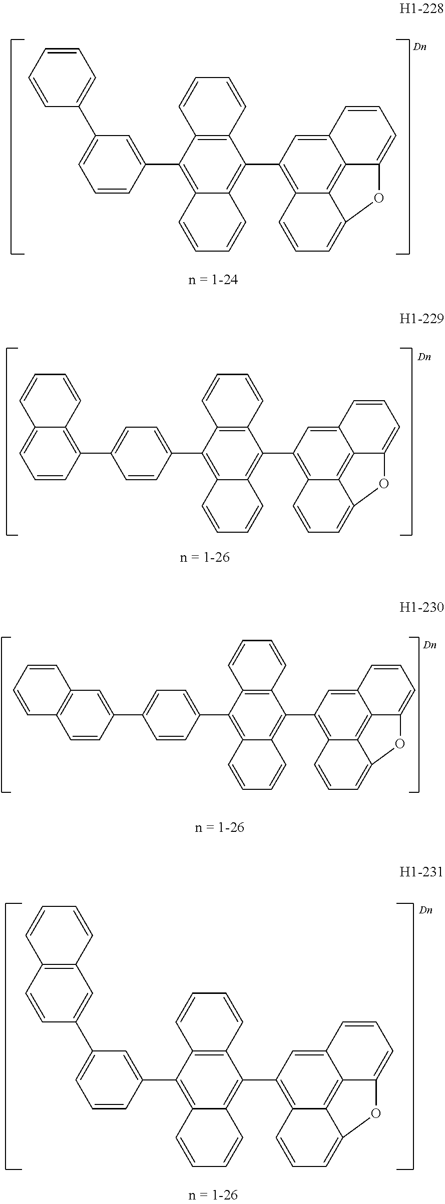 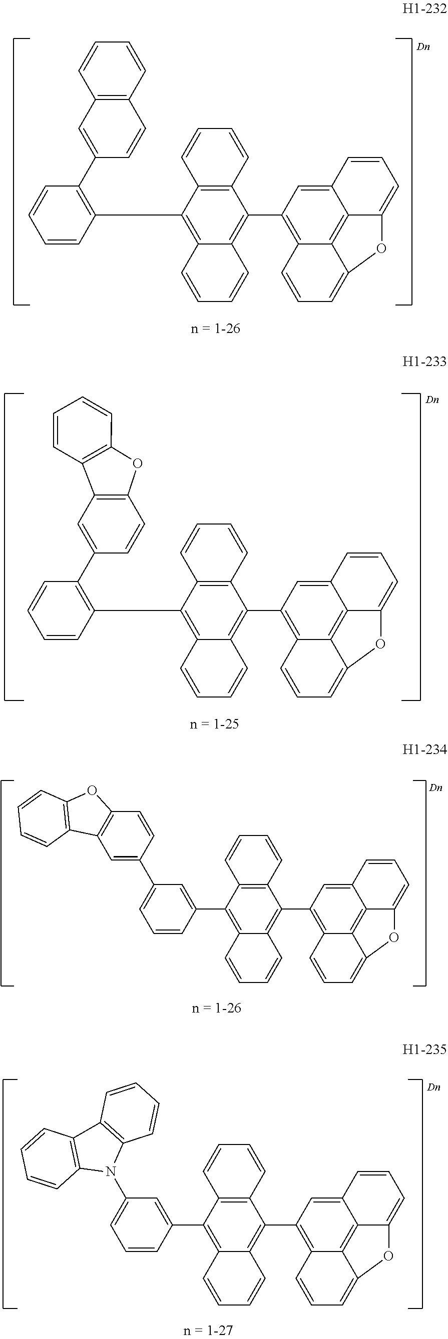 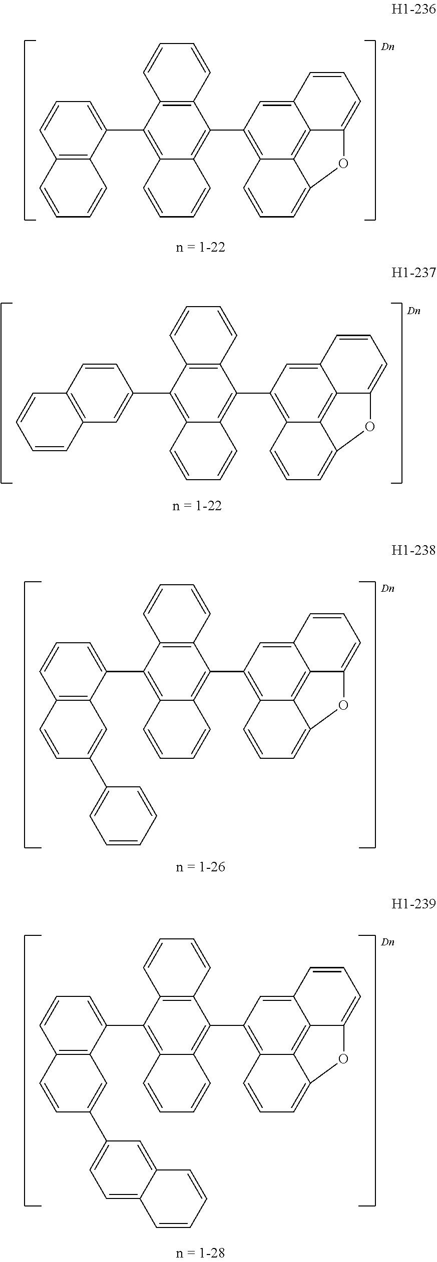 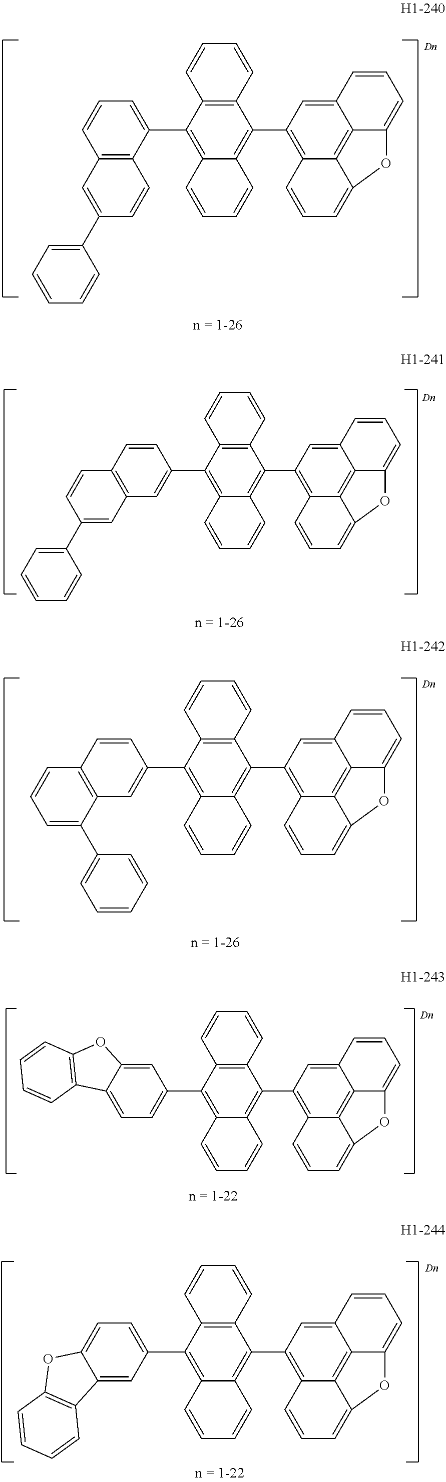 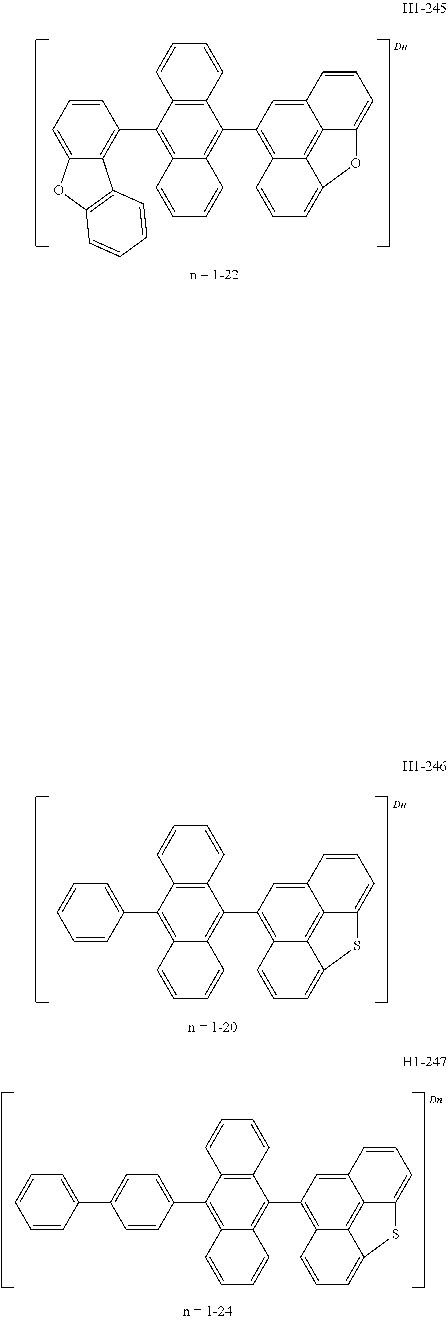 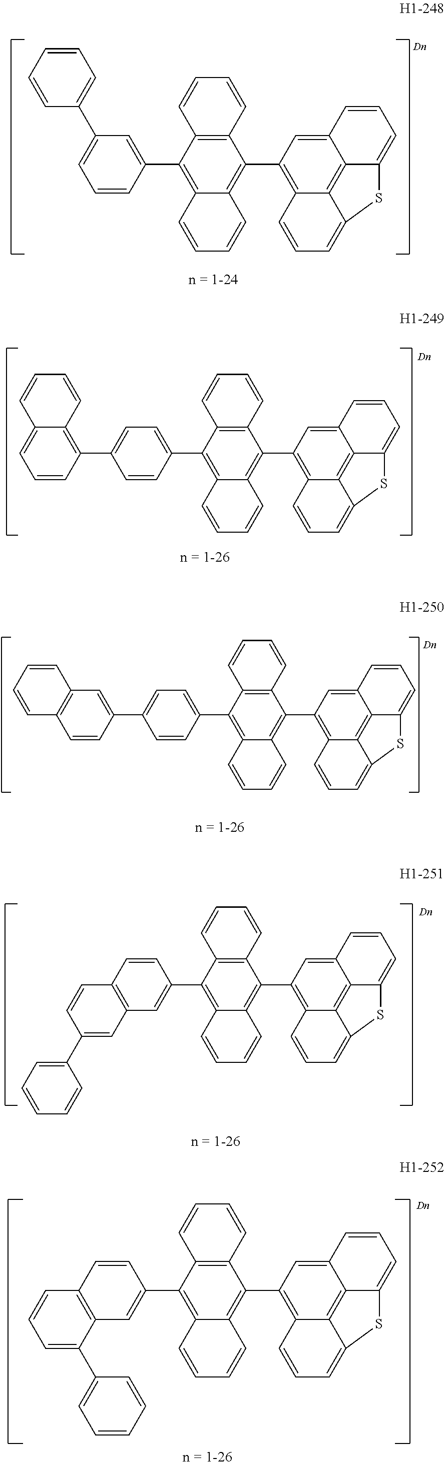 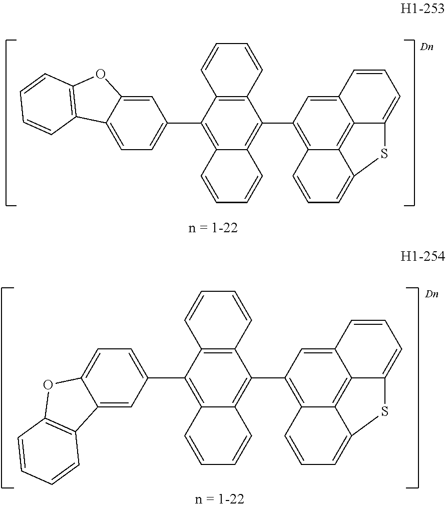 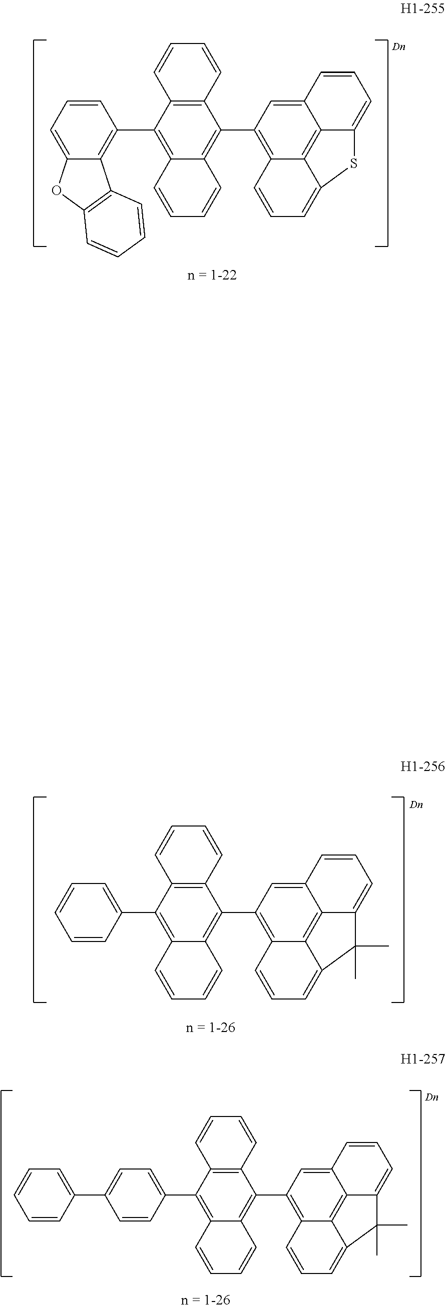 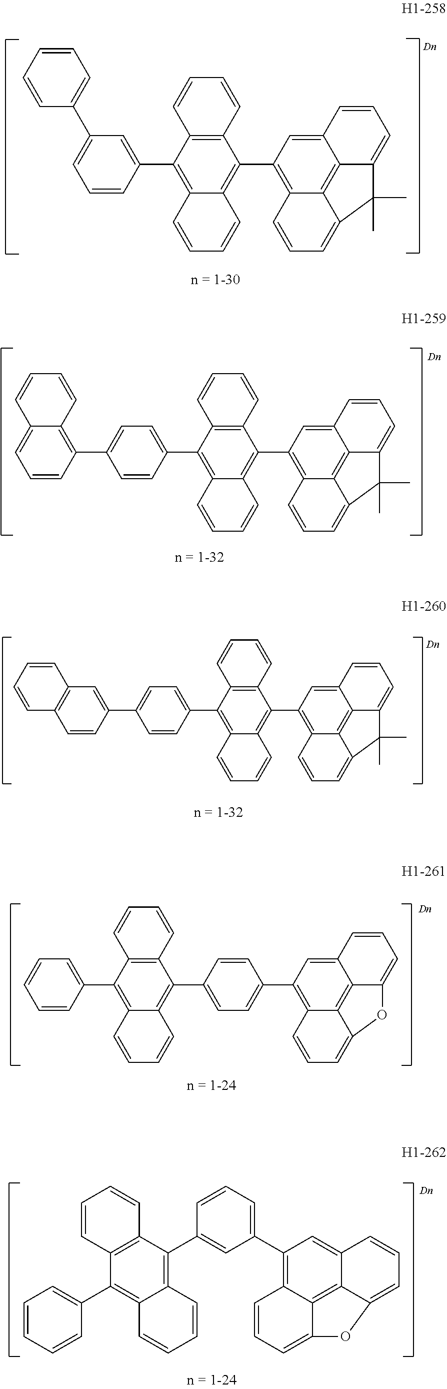 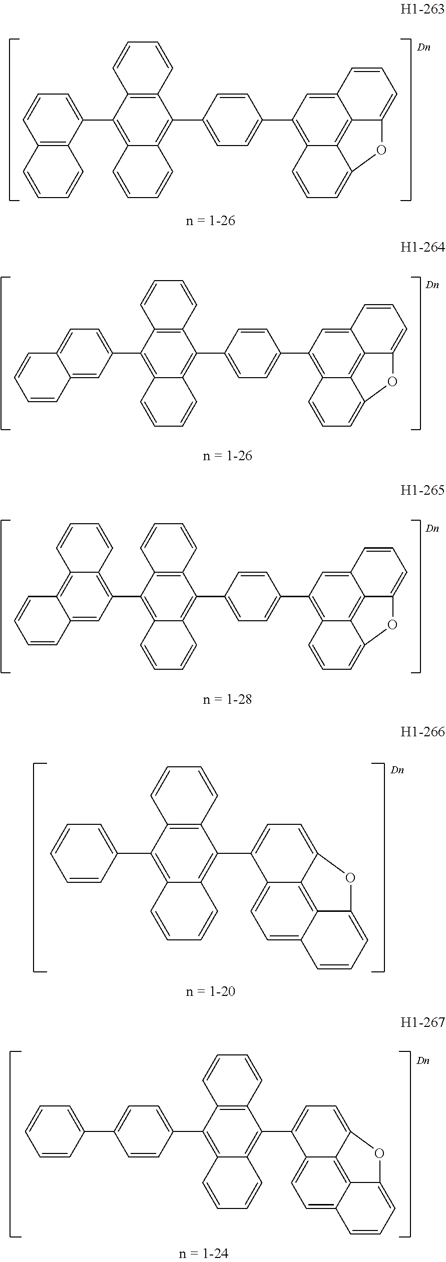 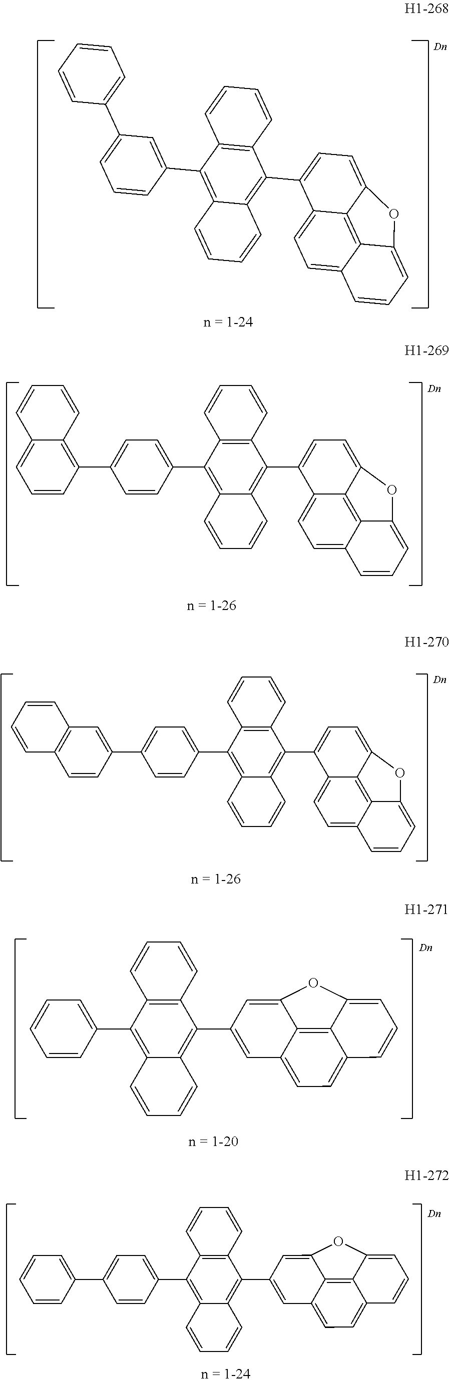 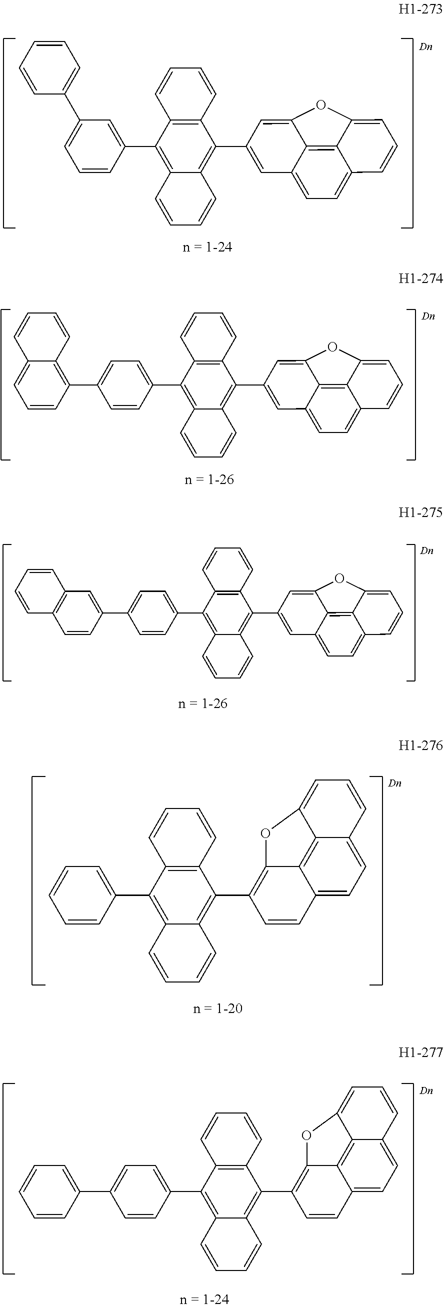 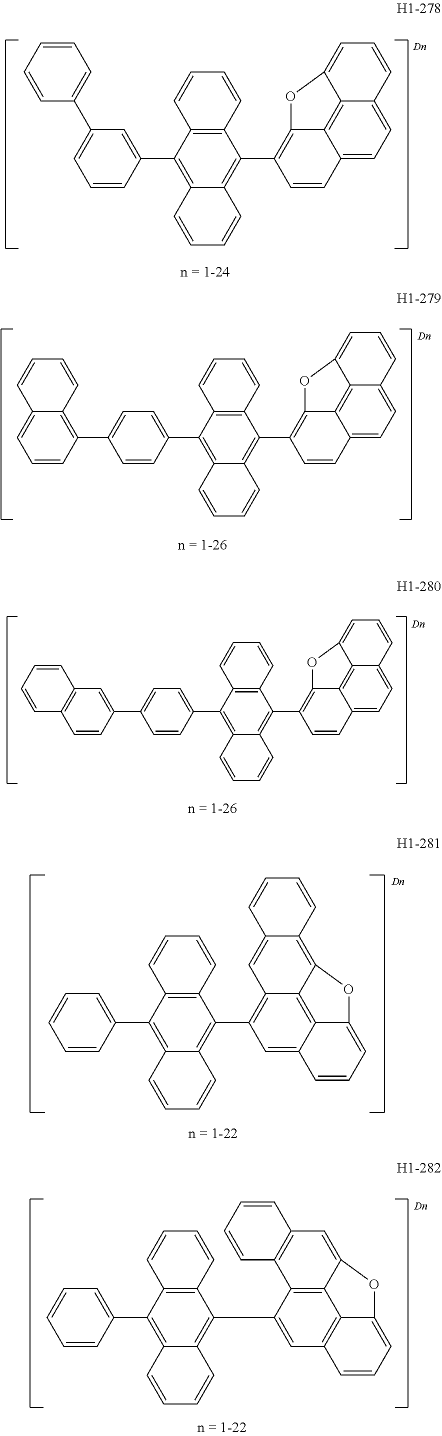 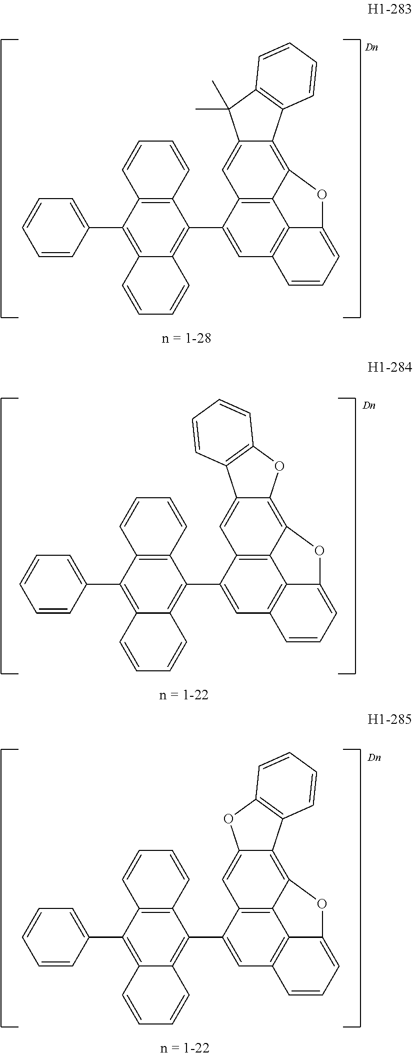 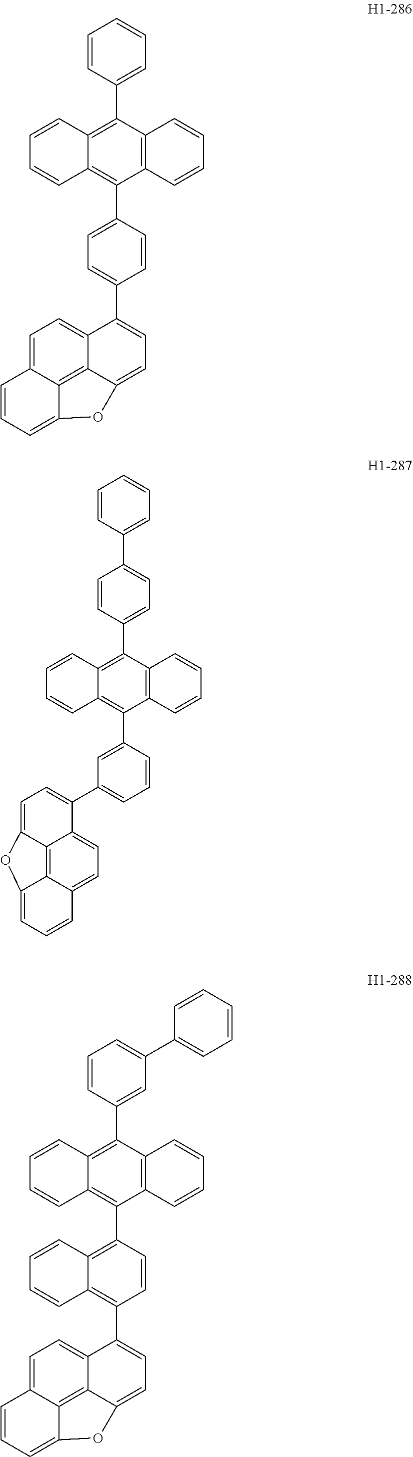 [SMILES: c1ccc(-c2c3ccccc3c(-c3ccc(-c4ccc5oc6cccc7ccc4c5c76)cc3)c3ccccc23)cc1 ; c1ccc(-c2ccc(-c3c4ccccc4c(-c4cccc(-c5ccc6oc7cccc8ccc5c6c87)c4)c4ccccc34)cc2)cc1 ; c1ccc(-c2cccc(-c3c4ccccc4c(-c4ccc(-c5ccc6oc7cccc8ccc5c6c87)c5ccccc45)c4ccccc34)c2)cc1] 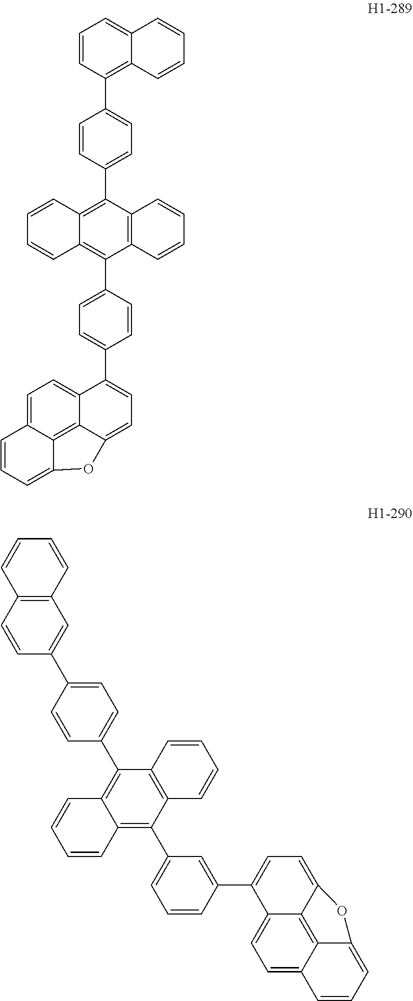 [SMILES: c1ccc2c(-c3ccc(-c4c5ccccc5c(-c5ccc(-c6ccc7oc8cccc9ccc6c7c98)cc5)c5ccccc45)cc3)cccc2c1 ; c1cc(-c2c3ccccc3c(-c3ccc(-c4ccc5ccccc5c4)cc3)c3ccccc23)cc(-c2ccc3oc4cccc5ccc2c3c54)c1] 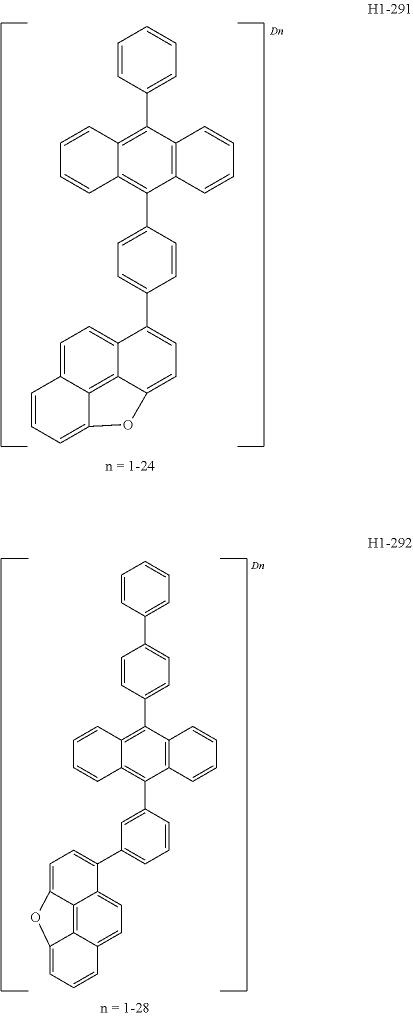 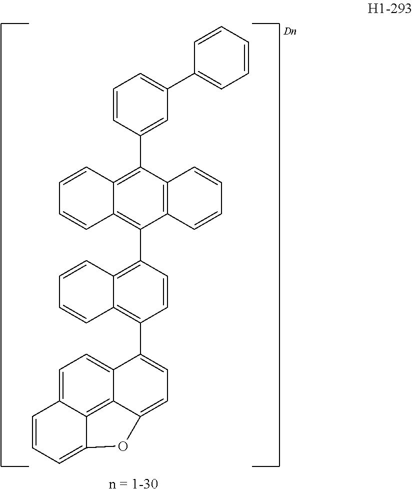 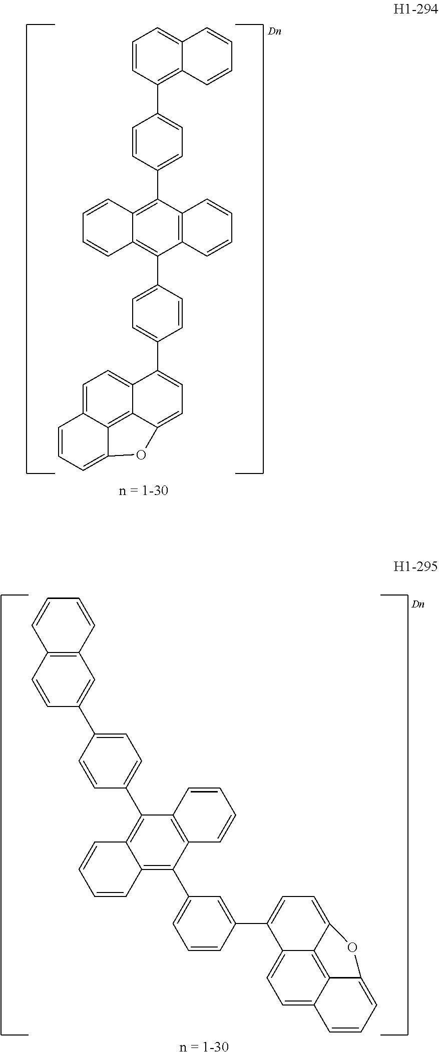 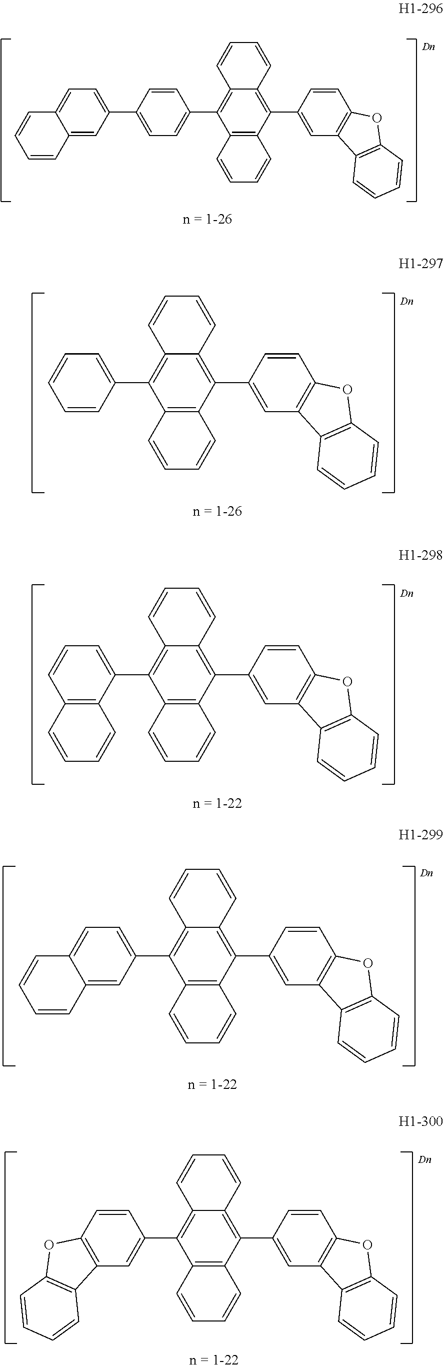 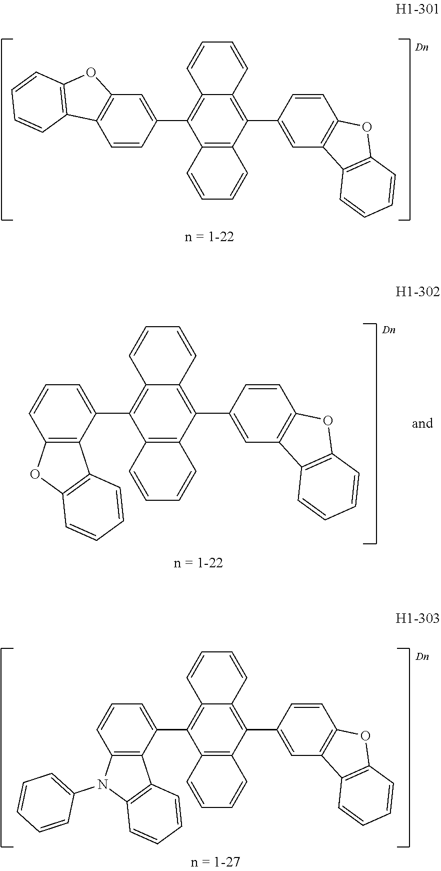
In the above compounds, Dn means that n hydrogens are replaced with deuterium, and n is from 1 to the maximum number of hydrogens in the compound.

10. The organic electroluminescent device of claim 5, wherein the compound represented by formulas 1-2 to 1-4 is selected from the following compounds:

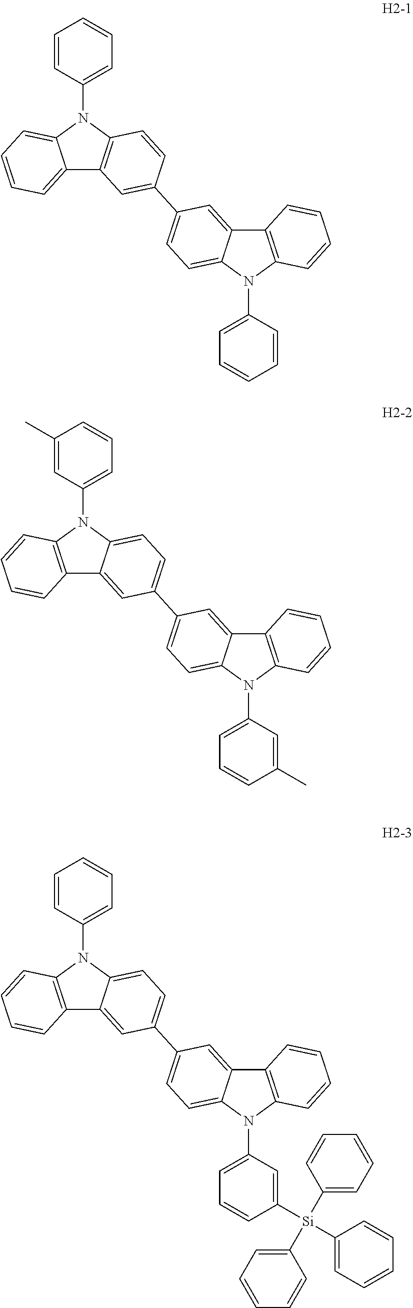 [SMILES: c1ccc(-n2c3ccccc3c3cc(-c4ccc5c(c4)c4ccccc4n5-c4ccccc4)ccc32)cc1 ; Cc1cccc(-n2c3ccccc3c3cc(-c4ccc5c(c4)c4ccccc4n5-c4cccc(C)c4)ccc32)c1 ; c1ccc(-n2c3ccccc3c3cc(-c4ccc5c(c4)c4ccccc4n5-c4cccc([Si](c5ccccc5)(c5ccccc5)c5ccccc5)c4)ccc32)cc1]

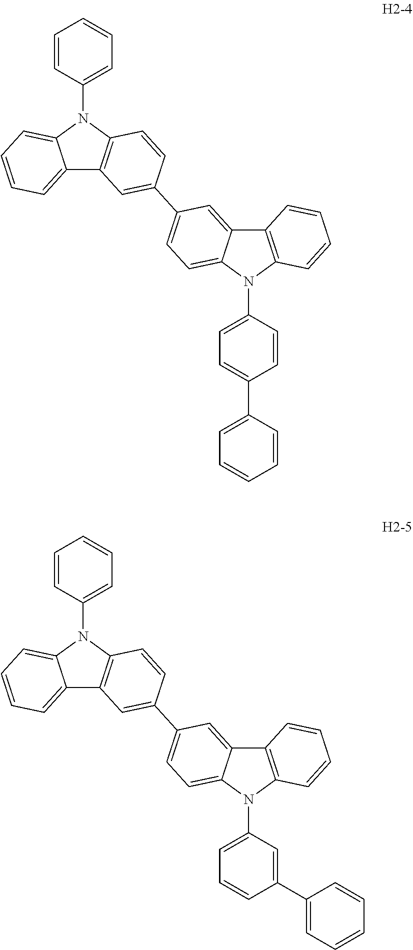 [SMILES: c1ccc(-c2ccc(-n3c4ccccc4c4cc(-c5ccc6c(c5)c5ccccc5n6-c5ccccc5)ccc43)cc2)cc1 ; c1ccc(-c2cccc(-n3c4ccccc4c4cc(-c5ccc6c(c5)c5ccccc5n6-c5ccccc5)ccc43)c2)cc1]

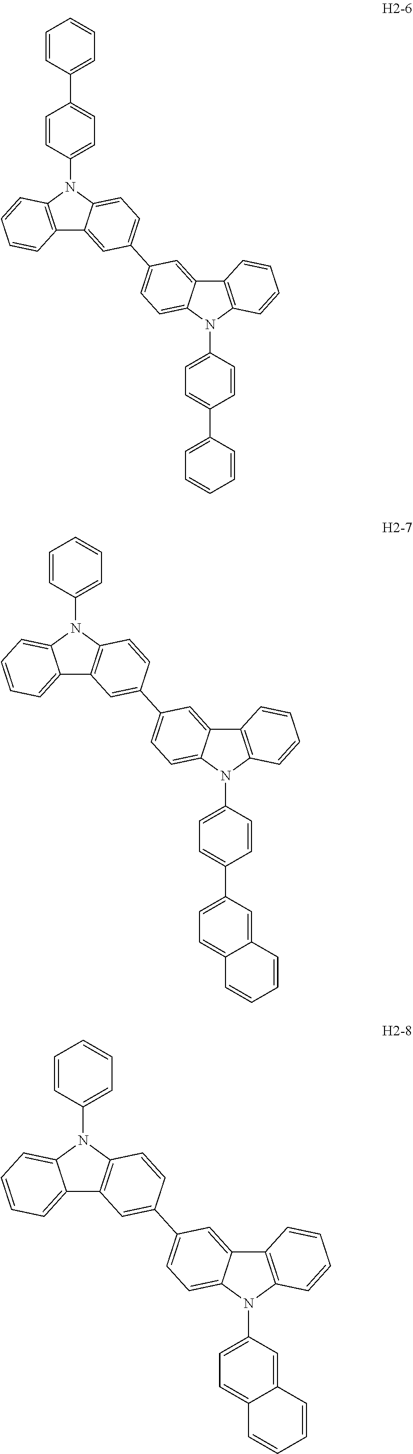 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-n2c3ccccc3c3cc(-c4ccc5c(c4)c4ccccc4n5-c4ccc(-c5ccc6ccccc6c5)cc4)ccc32)cc1 ; c1ccc(-n2c3ccccc3c3cc(-c4ccc5c(c4)c4ccccc4n5-c4ccc5ccccc5c4)ccc32)cc1]

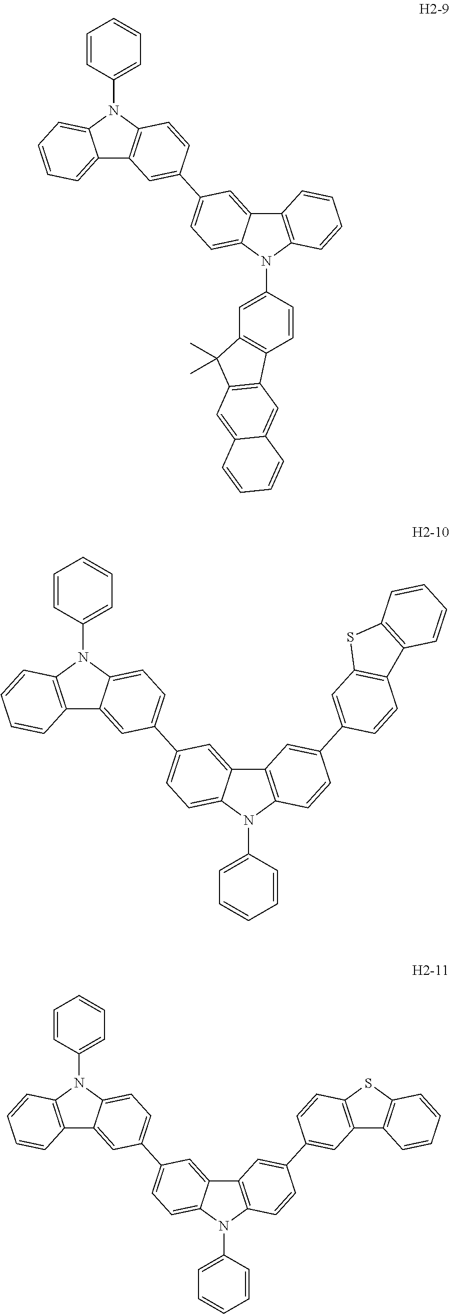 [SMILES: CC1(C)c2cc(-n3c4ccccc4c4cc(-c5ccc6c(c5)c5ccccc5n6-c5ccccc5)ccc43)ccc2-c2cc3ccccc3cc21 ; c1ccc(-n2c3ccccc3c3cc(-c4ccc5c(c4)c4cc(-c6ccc7c(c6)sc6ccccc67)ccc4n5-c4ccccc4)ccc32)cc1 ; c1ccc(-n2c3ccccc3c3cc(-c4ccc5c(c4)c4cc(-c6ccc7sc8ccccc8c7c6)ccc4n5-c4ccccc4)ccc32)cc1]

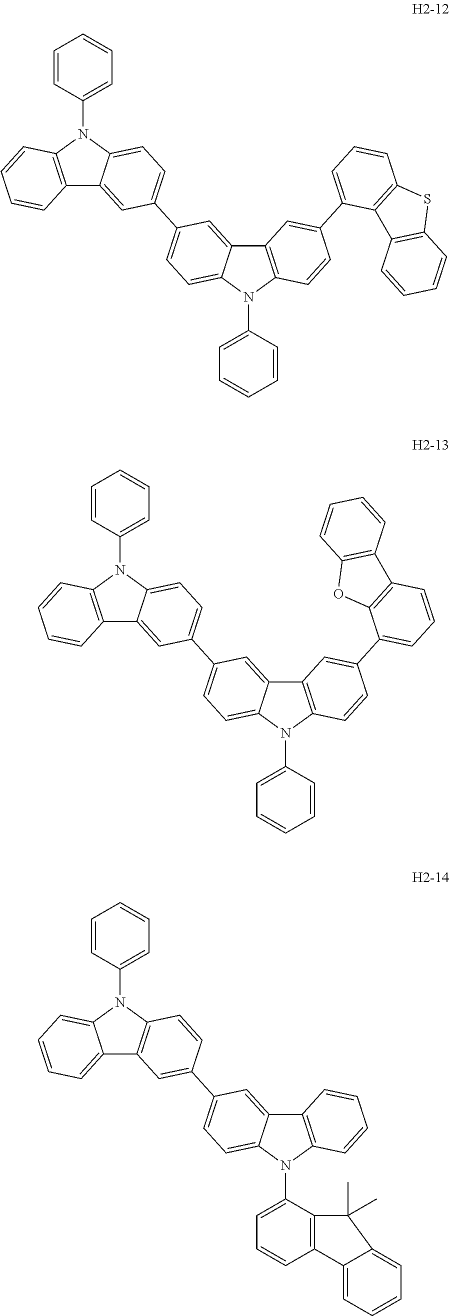 [SMILES: c1ccc(-n2c3ccccc3c3cc(-c4ccc5c(c4)c4cc(-c6cccc7sc8ccccc8c67)ccc4n5-c4ccccc4)ccc32)cc1 ; c1ccc(-n2c3ccccc3c3cc(-c4ccc5c(c4)c4cc(-c6cccc7c6oc6ccccc67)ccc4n5-c4ccccc4)ccc32)cc1 ; CC1(C)c2ccccc2-c2cccc(-n3c4ccccc4c4cc(-c5ccc6c(c5)c5ccccc5n6-c5ccccc5)ccc43)c21]

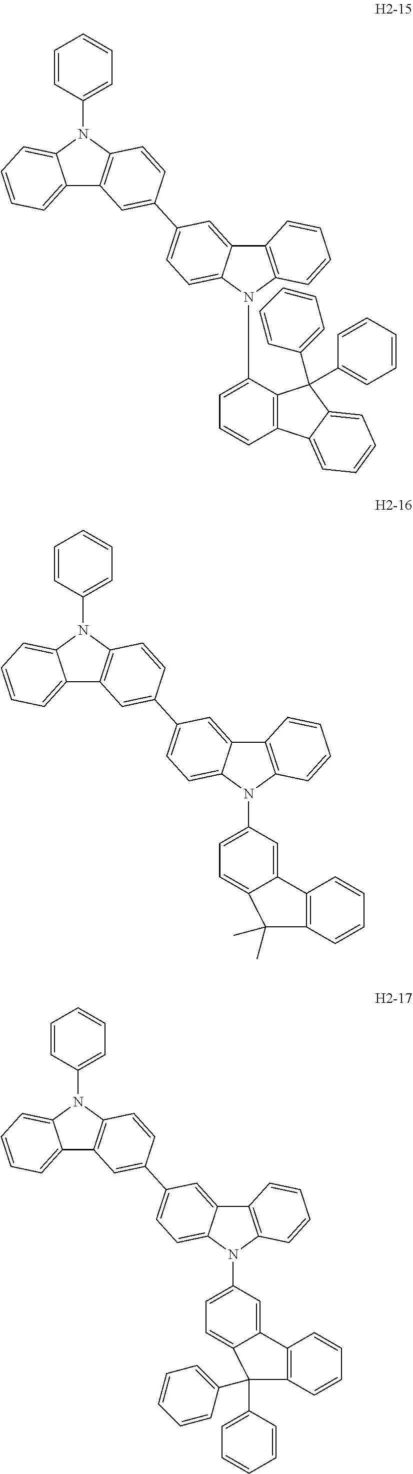 [SMILES: c1ccc(-n2c3ccccc3c3cc(-c4ccc5c(c4)c4ccccc4n5-c4cccc5c4C(c4ccccc4)(c4ccccc4)c4ccccc4-5)ccc32)cc1 ; CC1(C)c2ccccc2-c2cc(-n3c4ccccc4c4cc(-c5ccc6c(c5)c5ccccc5n6-c5ccccc5)ccc43)ccc21 ; c1ccc(-n2c3ccccc3c3cc(-c4ccc5c(c4)c4ccccc4n5-c4ccc5c(c4)-c4ccccc4C5(c4ccccc4)c4ccccc4)ccc32)cc1]

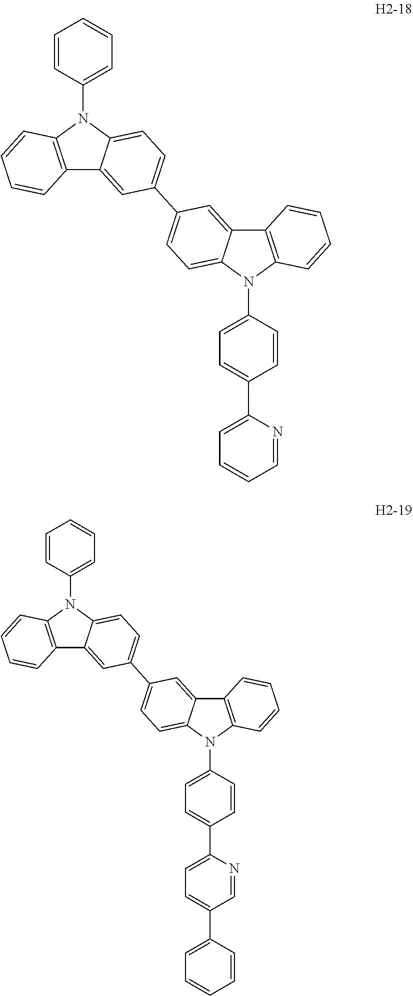 [SMILES: c1ccc(-n2c3ccccc3c3cc(-c4ccc5c(c4)c4ccccc4n5-c4ccc(-c5ccccn5)cc4)ccc32)cc1 ; c1ccc(-c2ccc(-c3ccc(-n4c5ccccc5c5cc(-c6ccc7c(c6)c6ccccc6n7-c6ccccc6)ccc54)cc3)nc2)cc1]

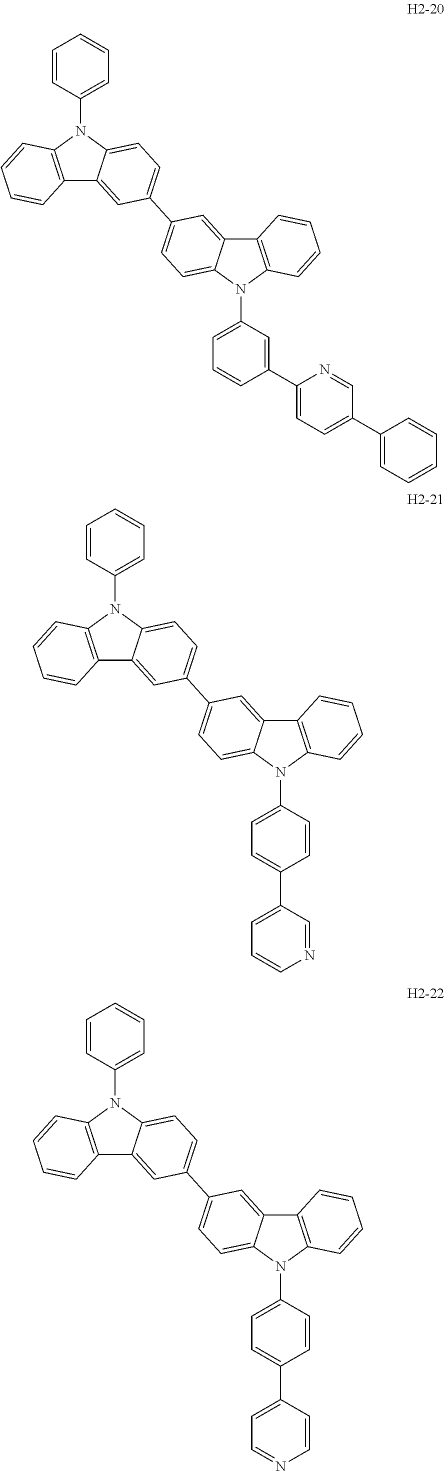 [SMILES: c1ccc(-c2ccc(-c3cccc(-n4c5ccccc5c5cc(-c6ccc7c(c6)c6ccccc6n7-c6ccccc6)ccc54)c3)nc2)cc1 ; c1ccc(-n2c3ccccc3c3cc(-c4ccc5c(c4)c4ccccc4n5-c4ccc(-c5cccnc5)cc4)ccc32)cc1 ; c1ccc(-n2c3ccccc3c3cc(-c4ccc5c(c4)c4ccccc4n5-c4ccc(-c5ccncc5)cc4)ccc32)cc1]

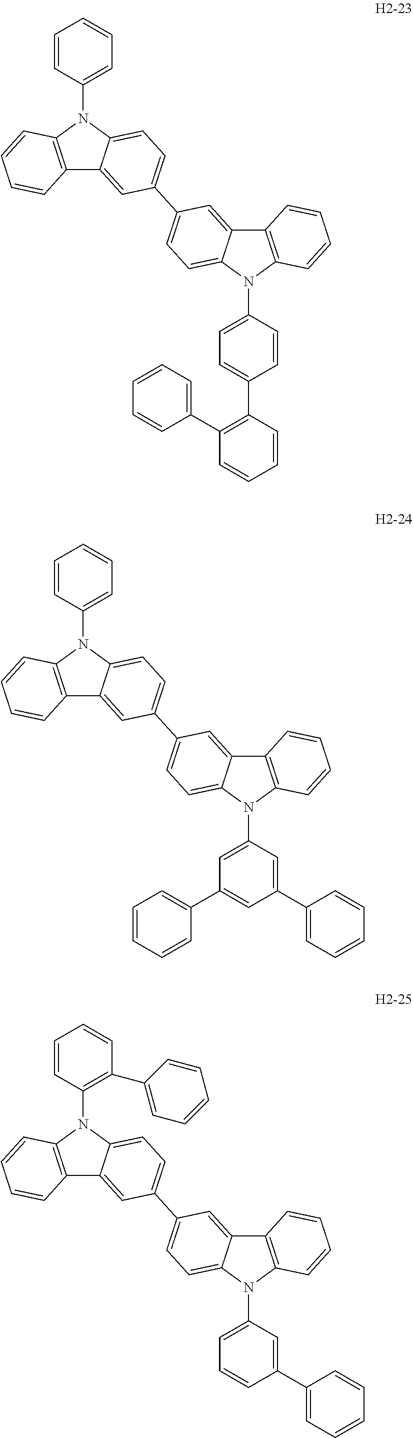 [SMILES: c1ccc(-c2ccccc2-c2ccc(-n3c4ccccc4c4cc(-c5ccc6c(c5)c5ccccc5n6-c5ccccc5)ccc43)cc2)cc1 ; c1ccc(-c2cc(-c3ccccc3)cc(-n3c4ccccc4c4cc(-c5ccc6c(c5)c5ccccc5n6-c5ccccc5)ccc43)c2)cc1 ; c1ccc(-c2cccc(-n3c4ccccc4c4cc(-c5ccc6c(c5)c5ccccc5n6-c5ccccc5-c5ccccc5)ccc43)c2)cc1]

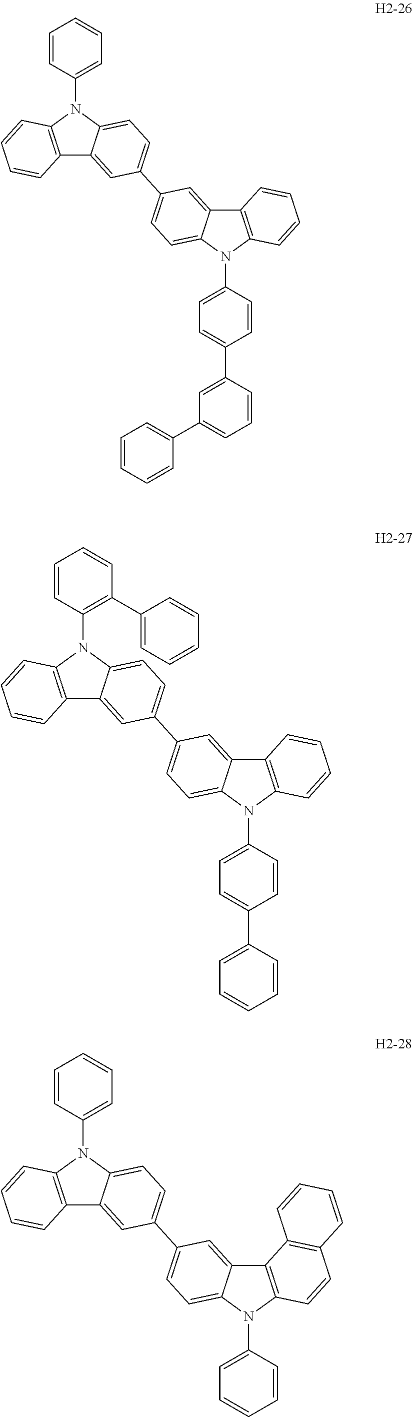 [SMILES: c1ccc(-c2cccc(-c3ccc(-n4c5ccccc5c5cc(-c6ccc7c(c6)c6ccccc6n7-c6ccccc6)ccc54)cc3)c2)cc1 ; c1ccc(-c2ccc(-n3c4ccccc4c4cc(-c5ccc6c(c5)c5ccccc5n6-c5ccccc5-c5ccccc5)ccc43)cc2)cc1 ; c1ccc(-n2c3ccccc3c3cc(-c4ccc5c(c4)c4c6ccccc6ccc4n5-c4ccccc4)ccc32)cc1]

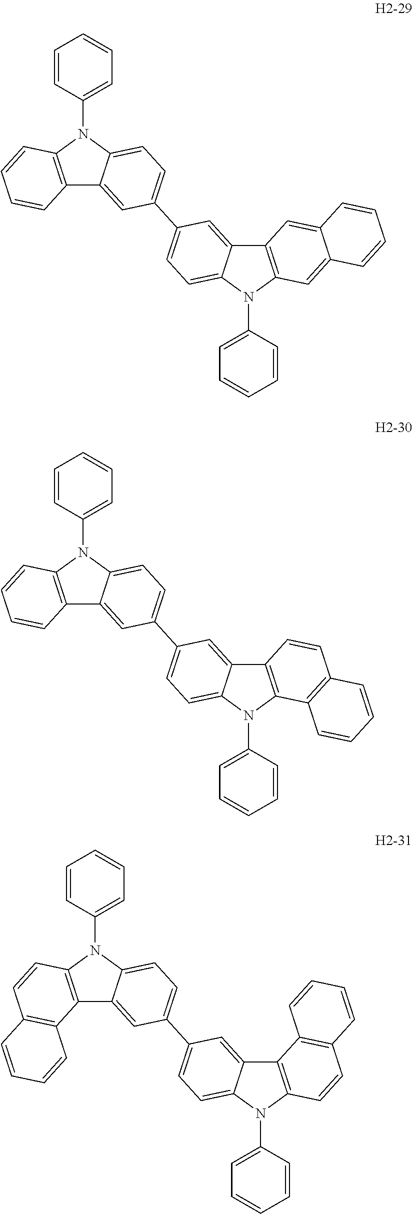 [SMILES: c1ccc(-n2c3ccccc3c3cc(-c4ccc5c(c4)c4cc6ccccc6cc4n5-c4ccccc4)ccc32)cc1 ; c1ccc(-n2c3ccccc3c3cc(-c4ccc5c(c4)c4ccc6ccccc6c4n5-c4ccccc4)ccc32)cc1 ; c1ccc(-n2c3ccc(-c4ccc5c(c4)c4c6ccccc6ccc4n5-c4ccccc4)cc3c3c4ccccc4ccc32)cc1]

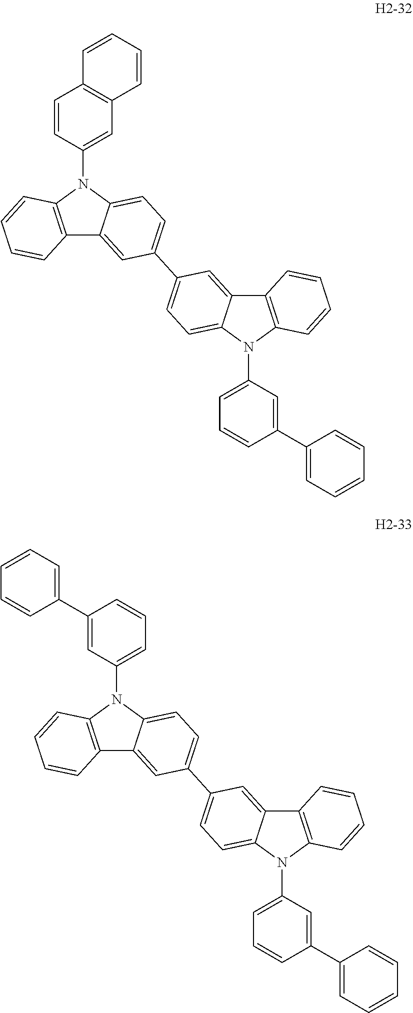 [SMILES: c1ccc(-c2cccc(-n3c4ccccc4c4cc(-c5ccc6c(c5)c5ccccc5n6-c5ccc6ccccc6c5)ccc43)c2)cc1 ; c1ccc(-c2cccc(-n3c4ccccc4c4cc(-c5ccc6c(c5)c5ccccc5n6-c5cccc(-c6ccccc6)c5)ccc43)c2)cc1]

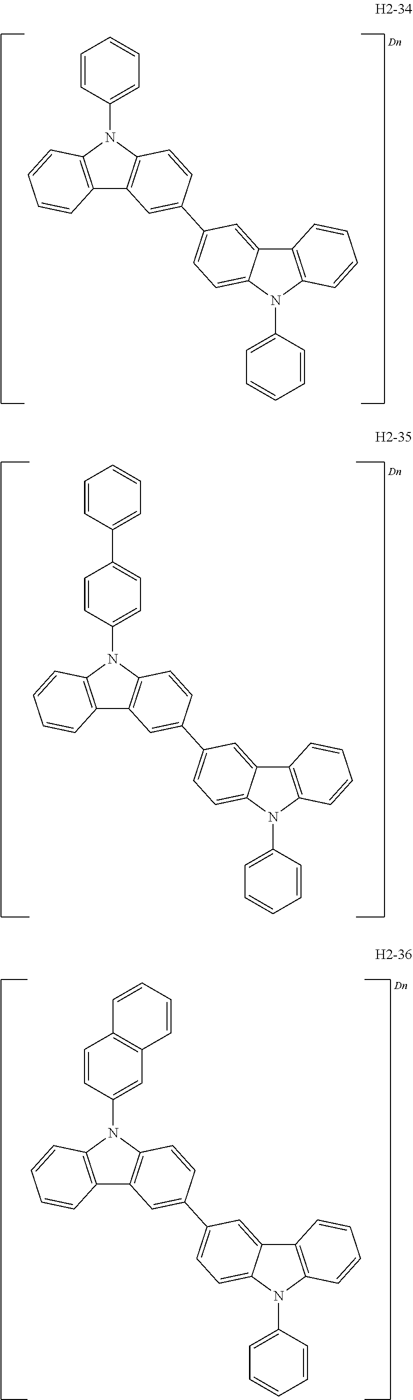

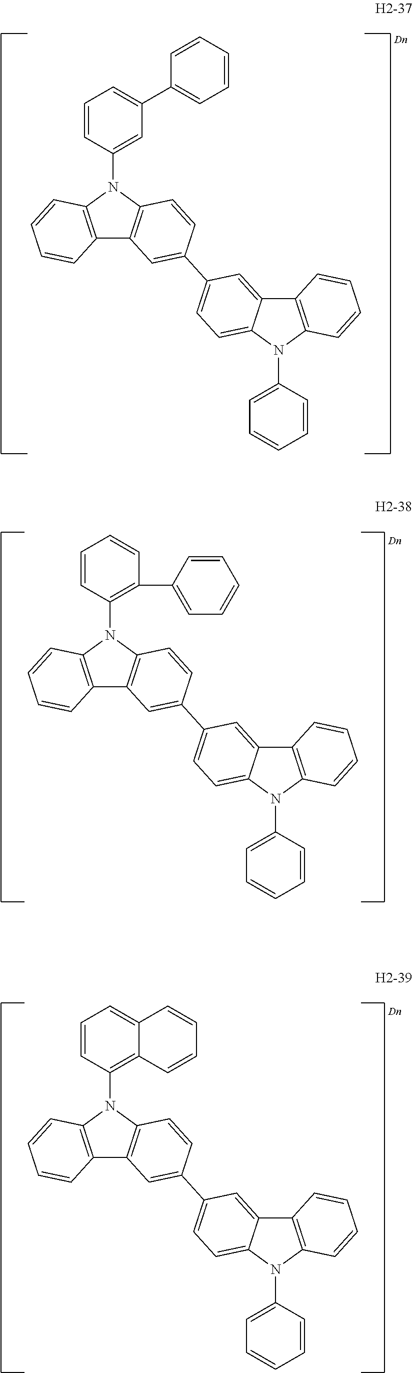

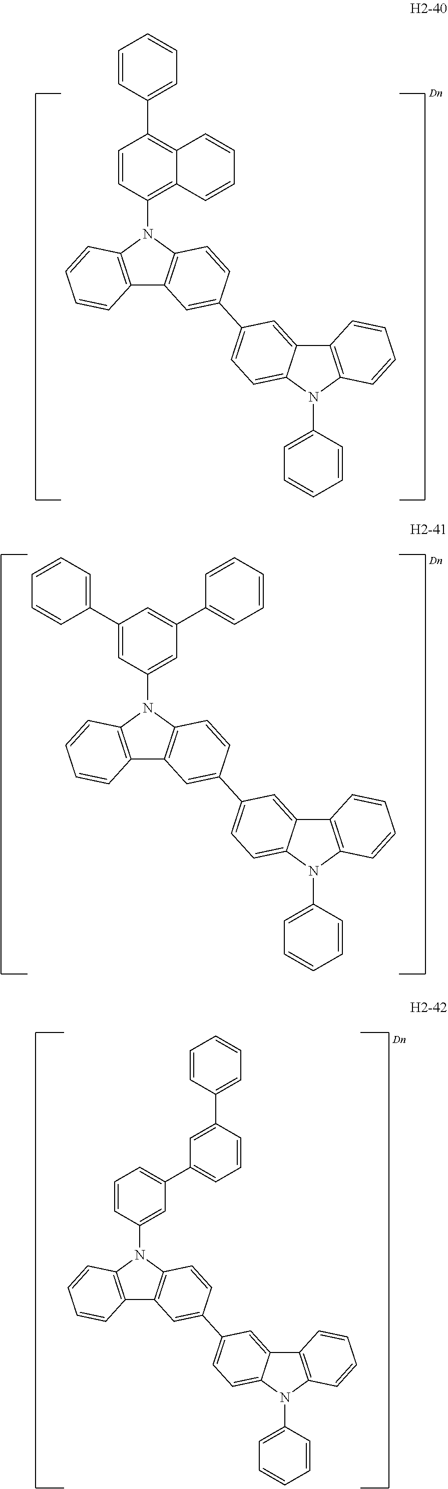

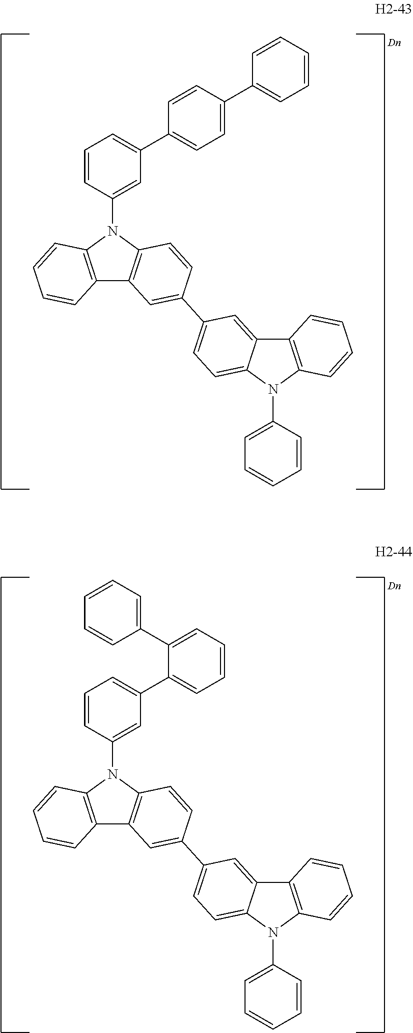

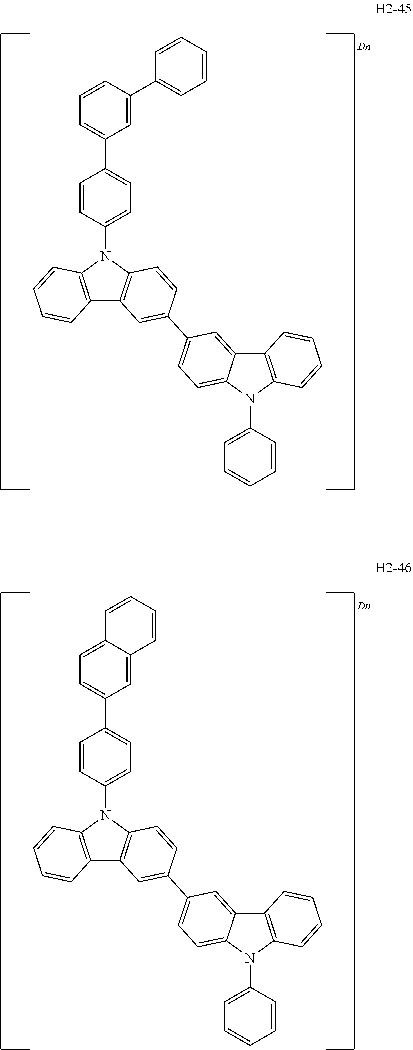

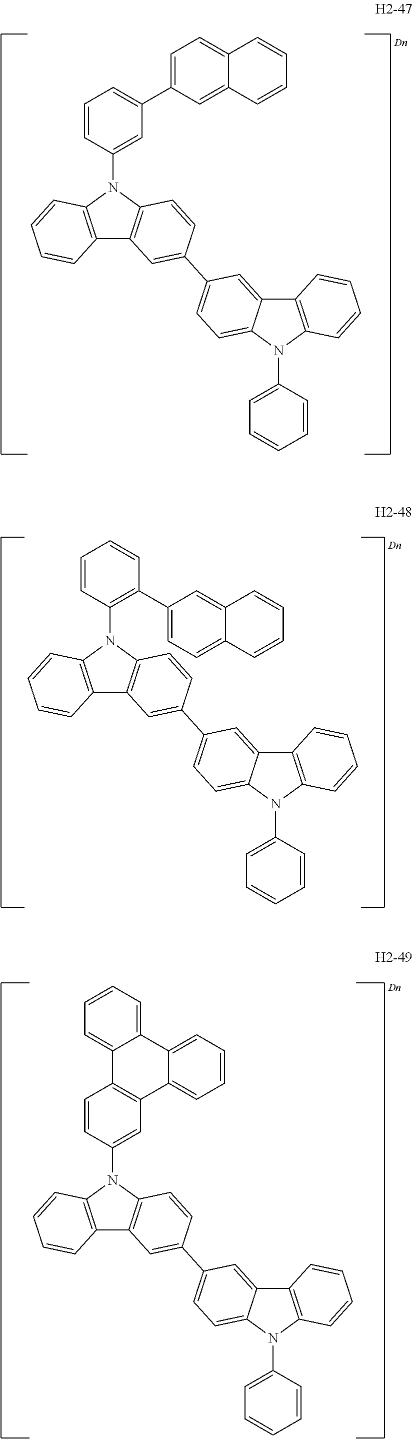

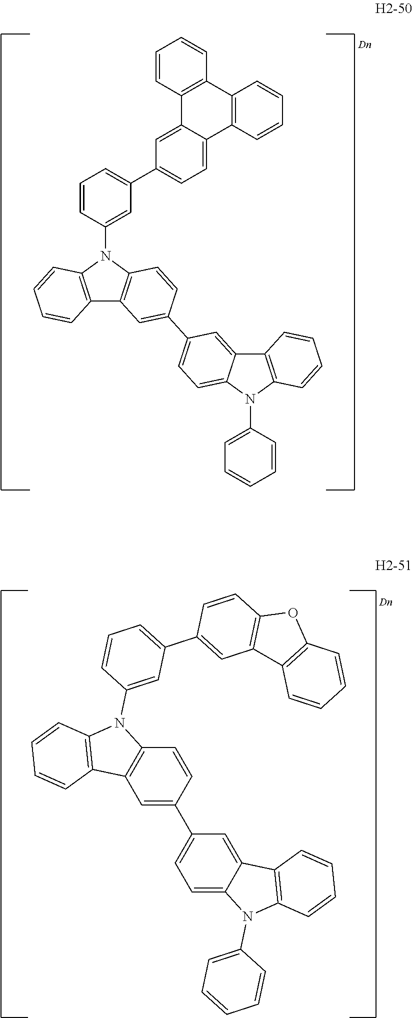

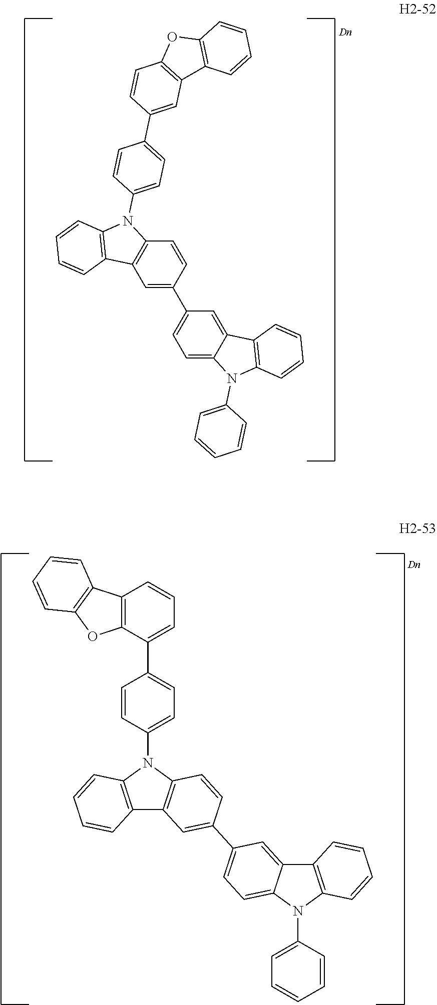

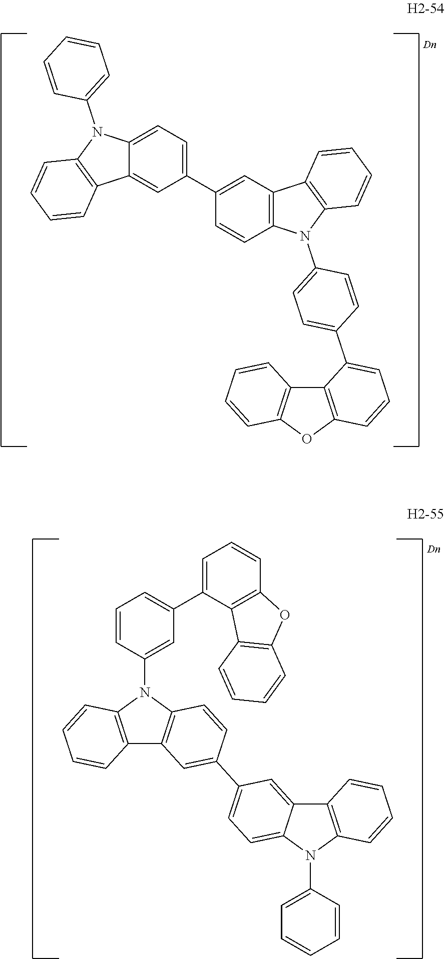

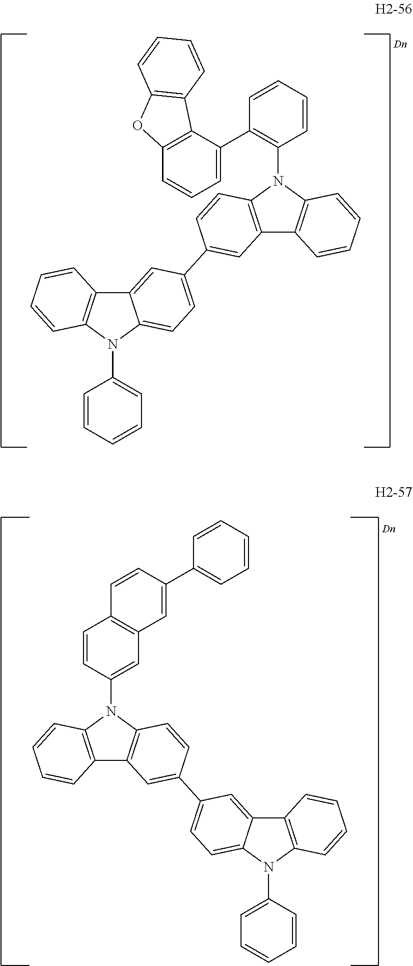

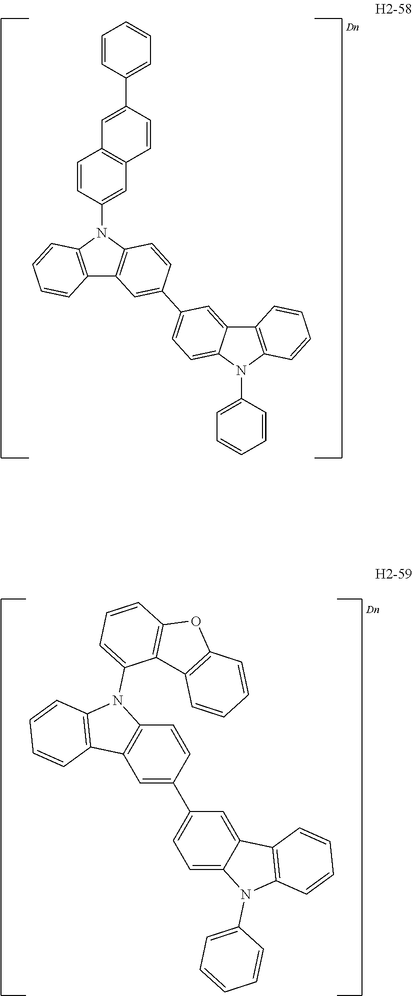

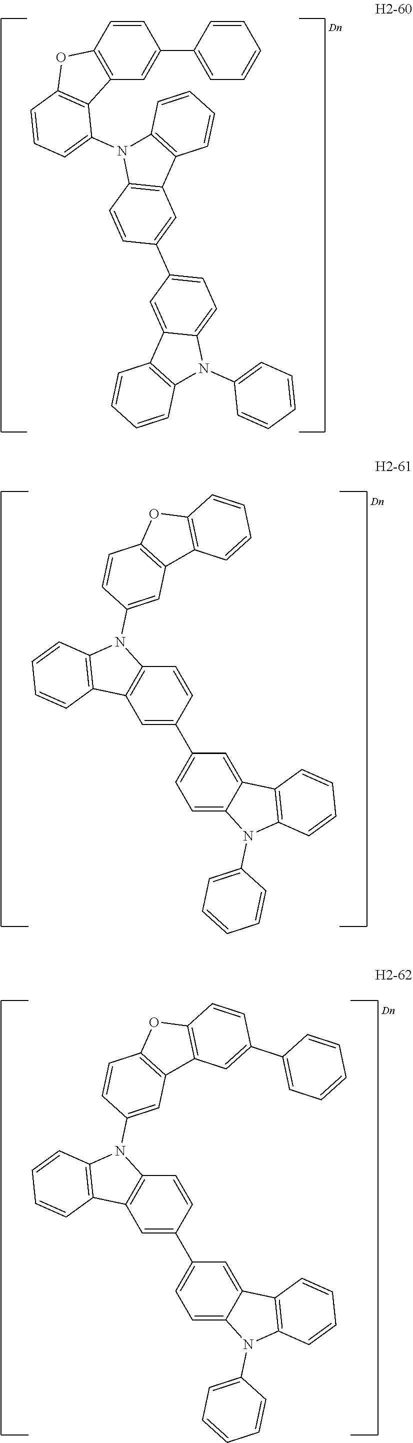

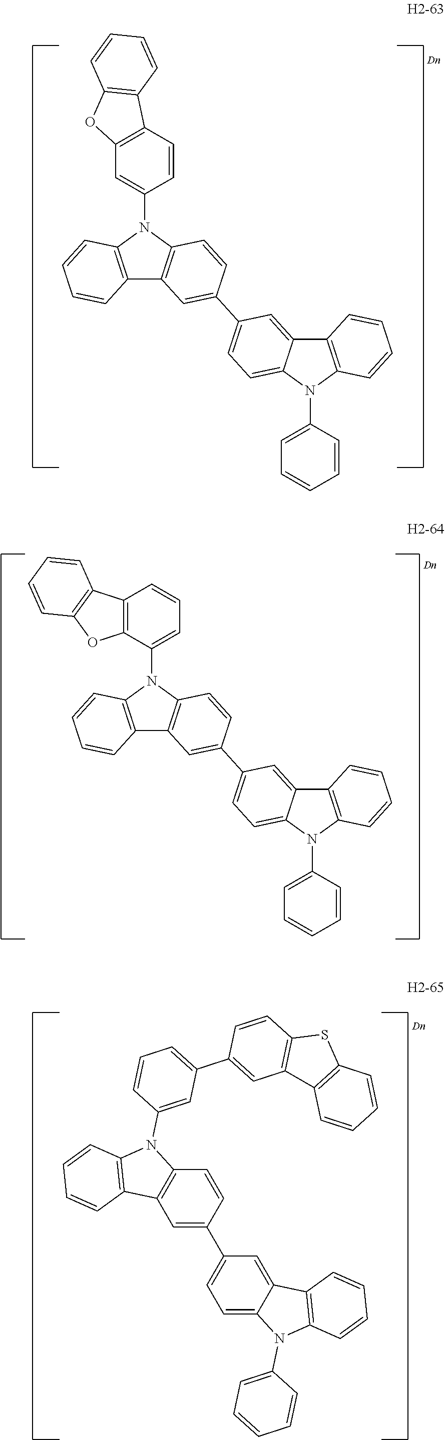

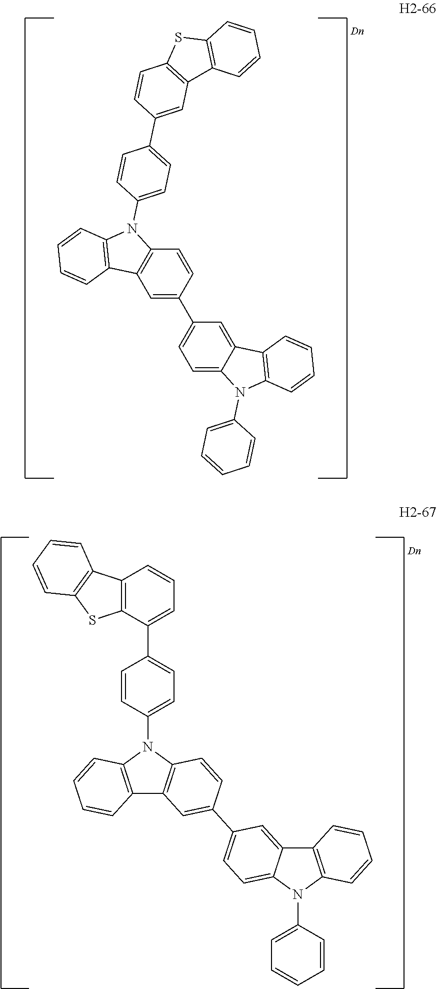

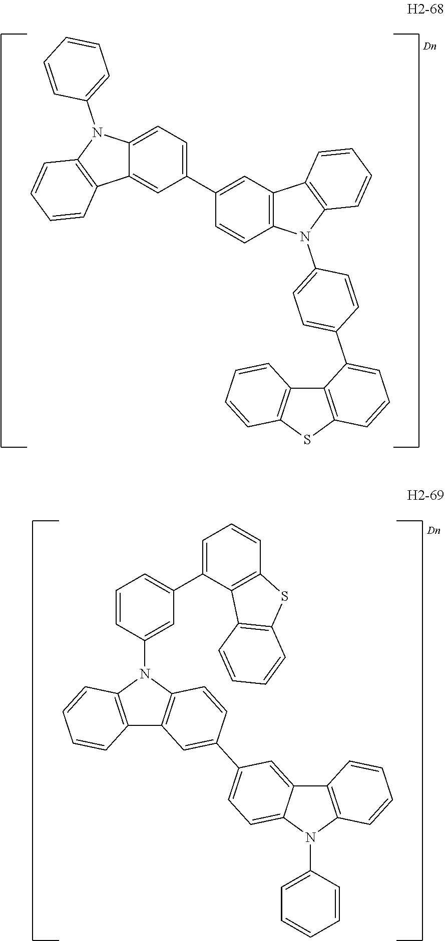

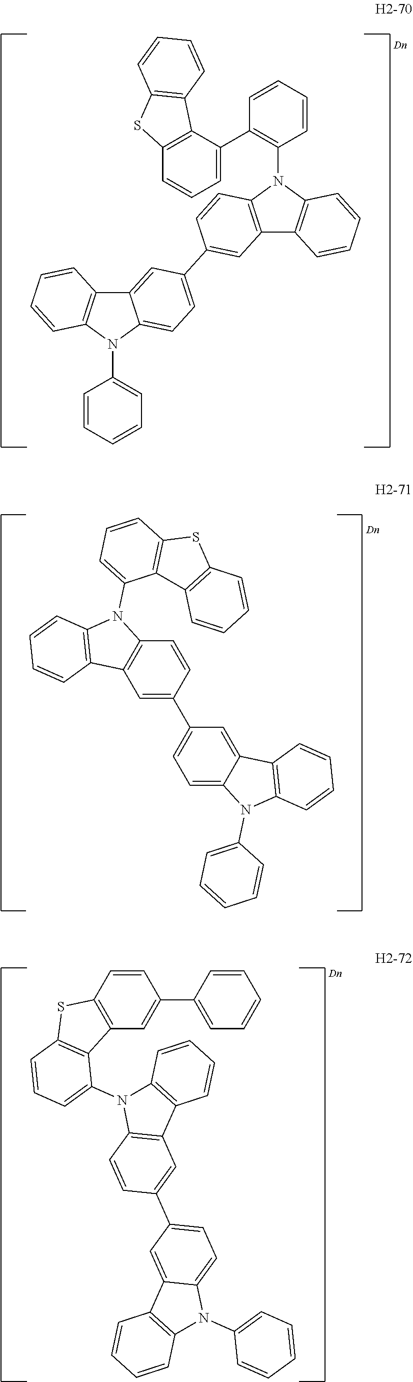

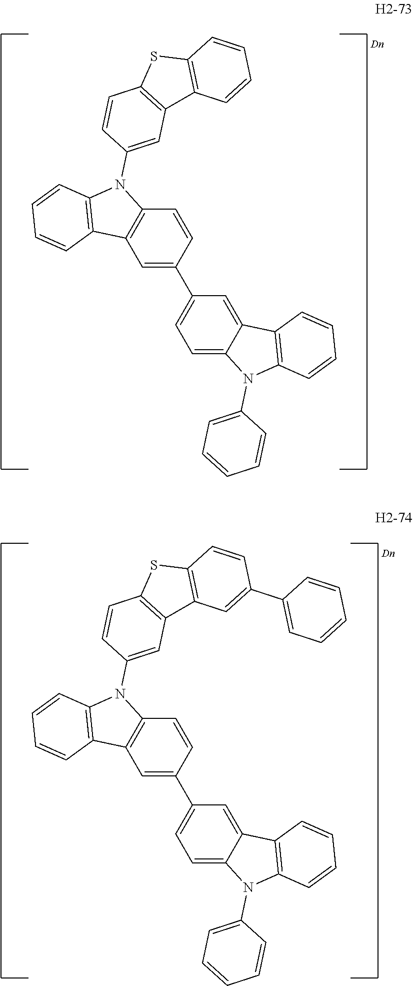

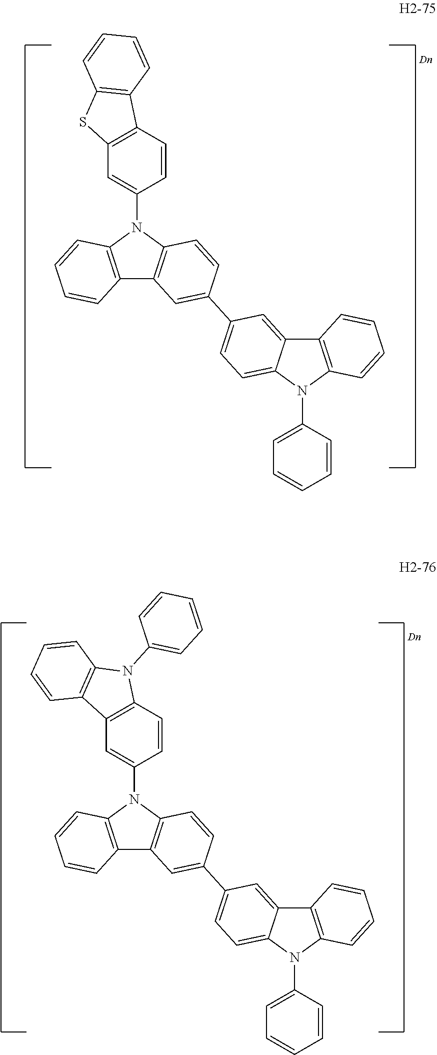

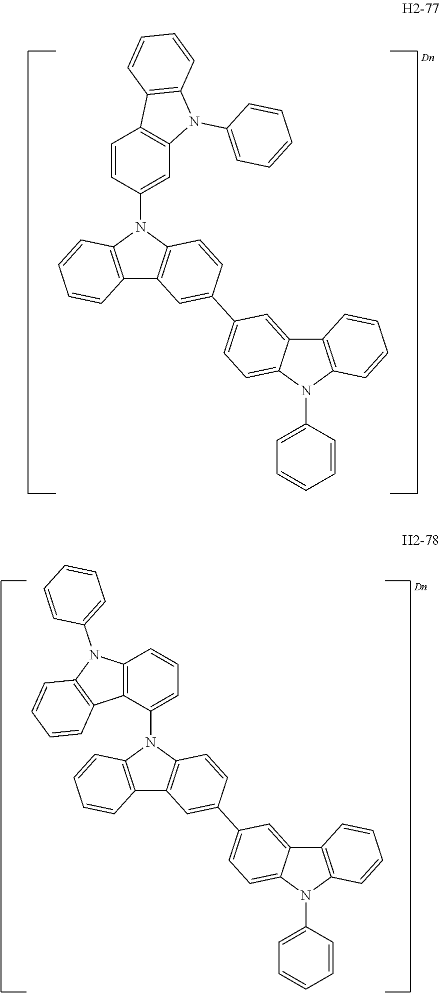

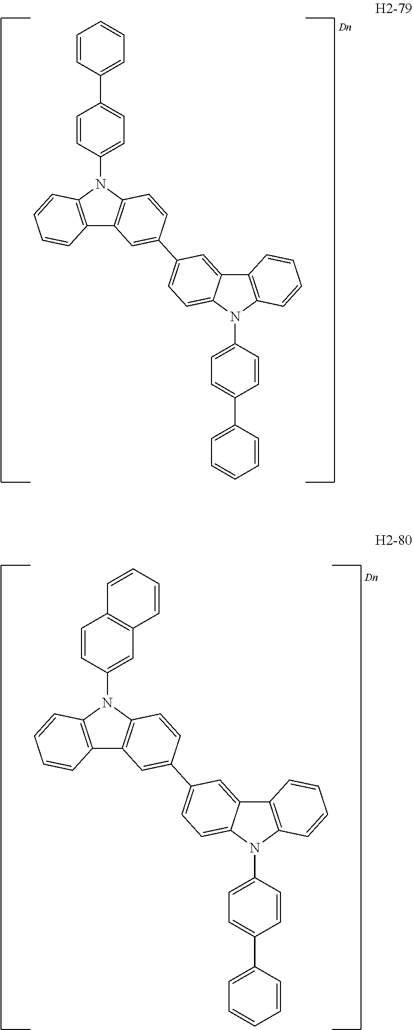

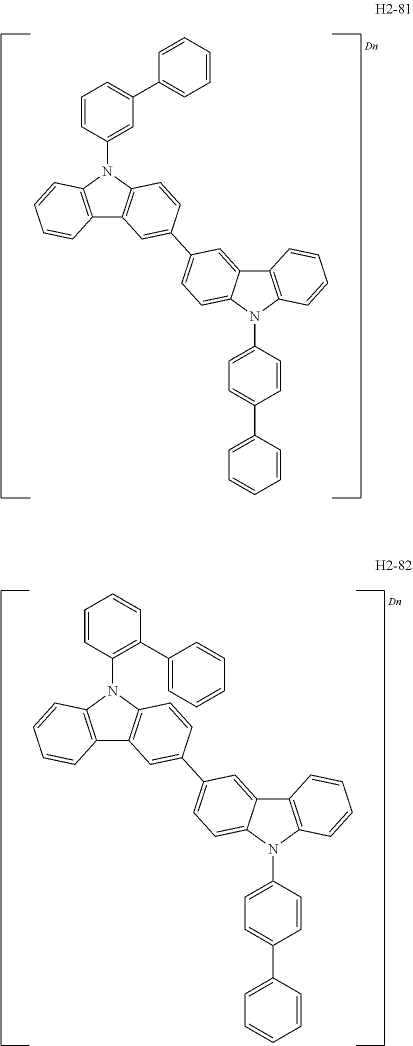

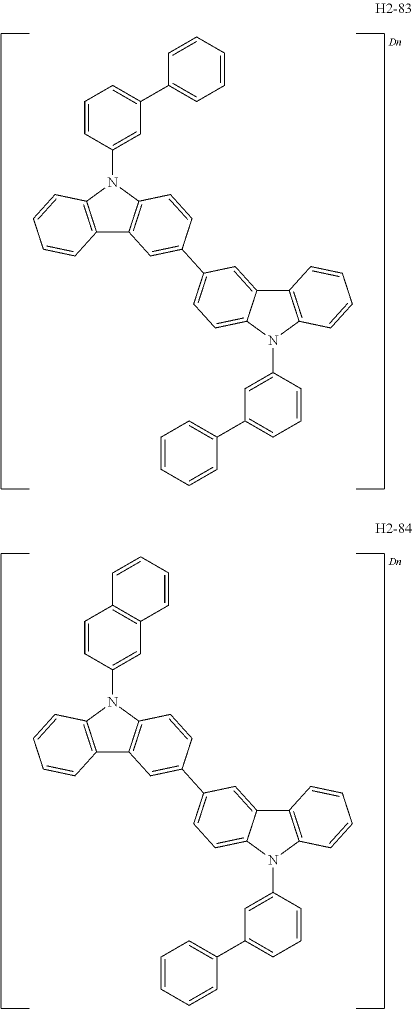

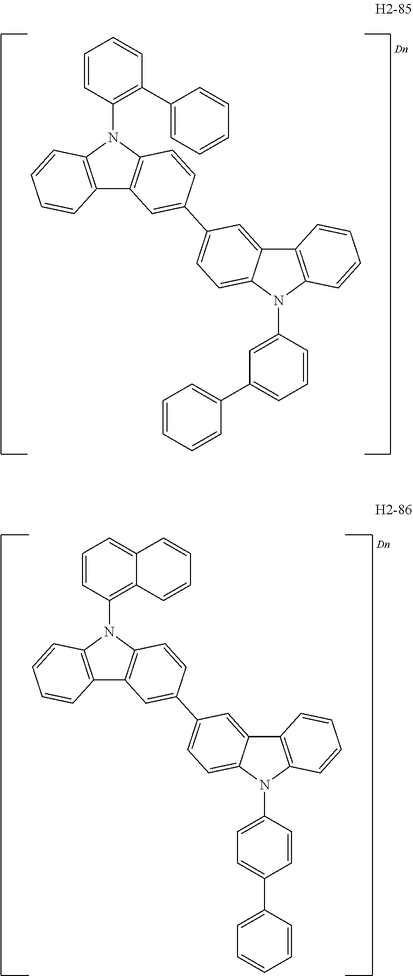

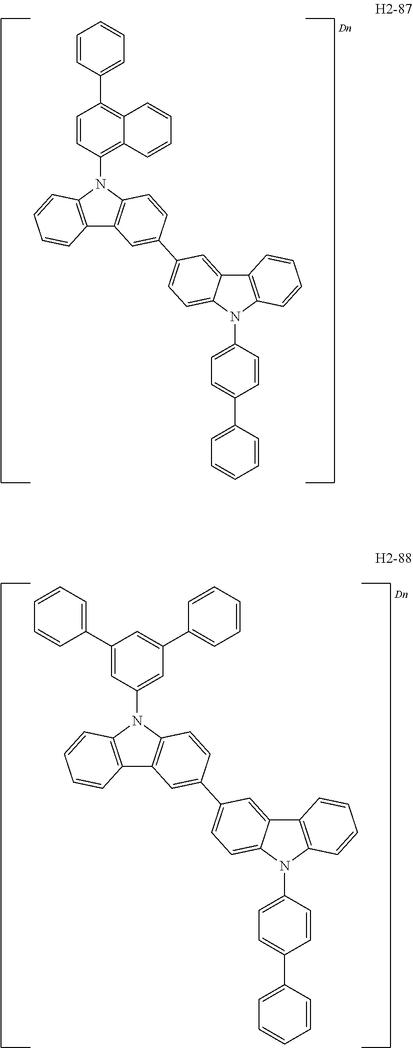

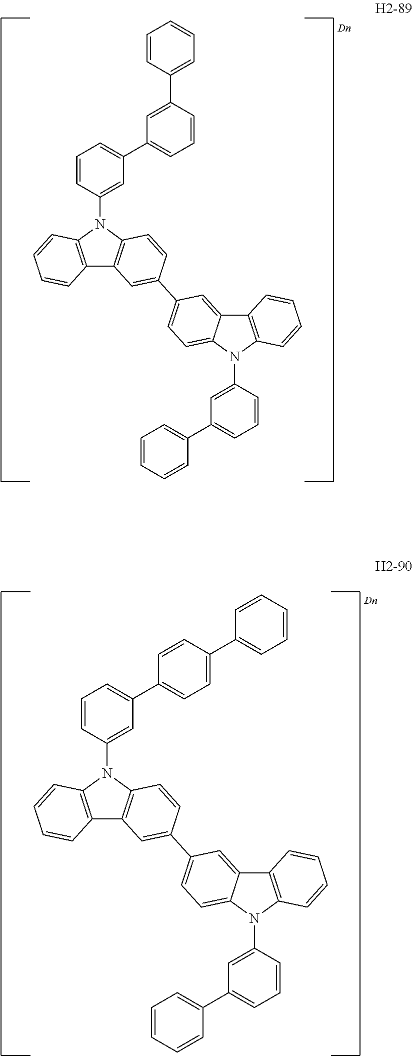

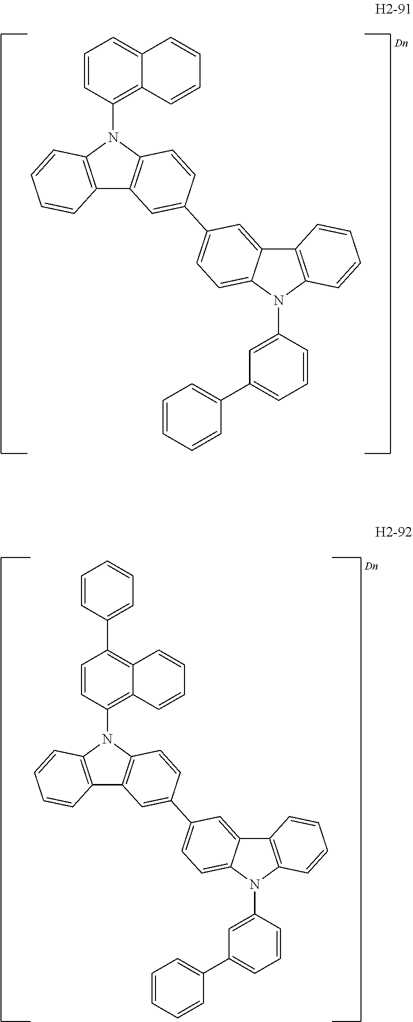

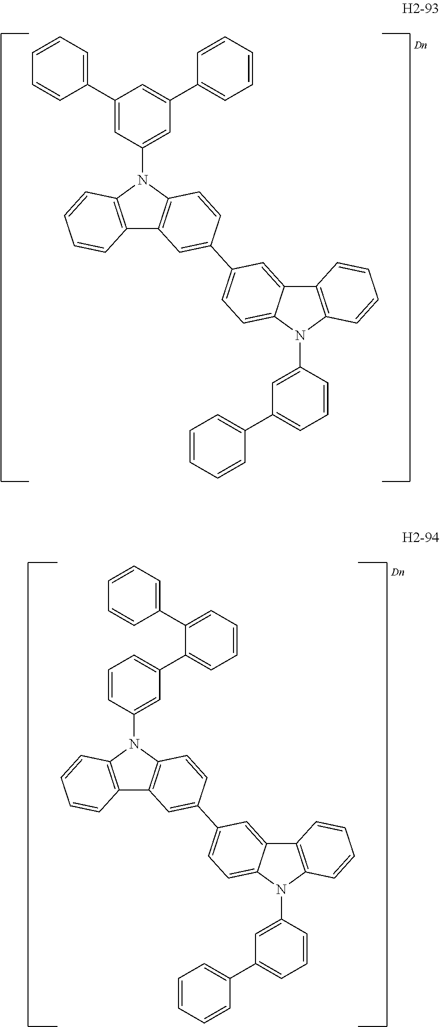

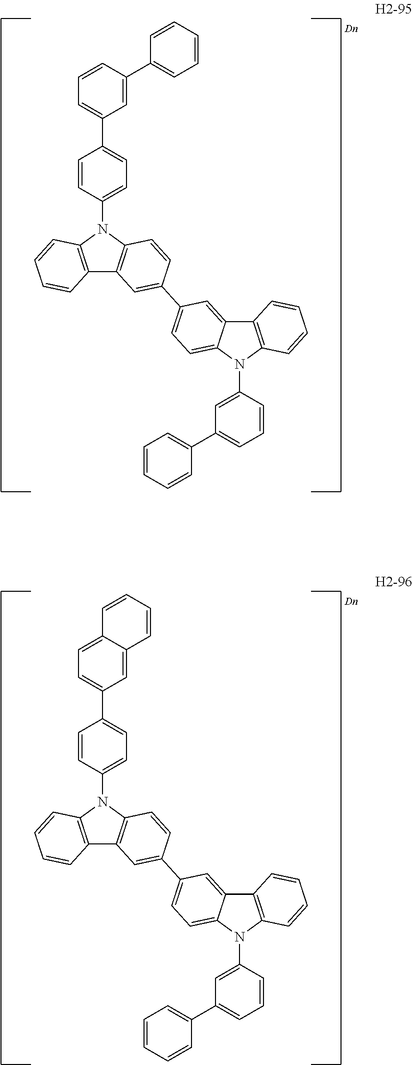

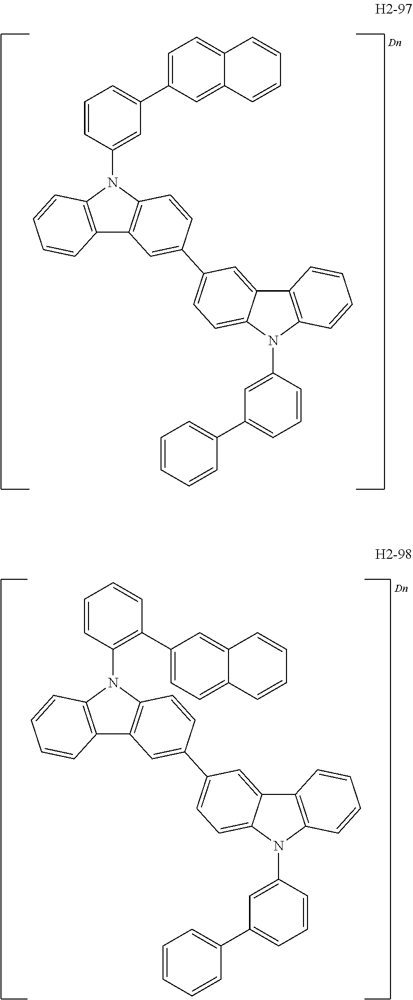

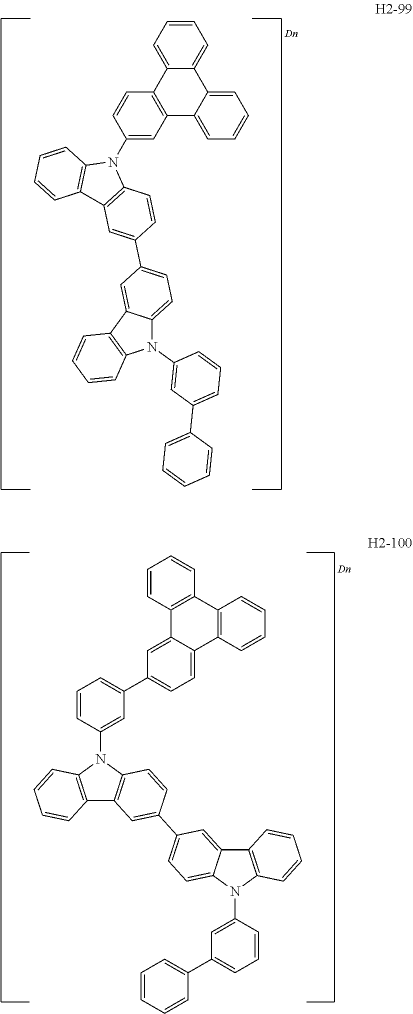

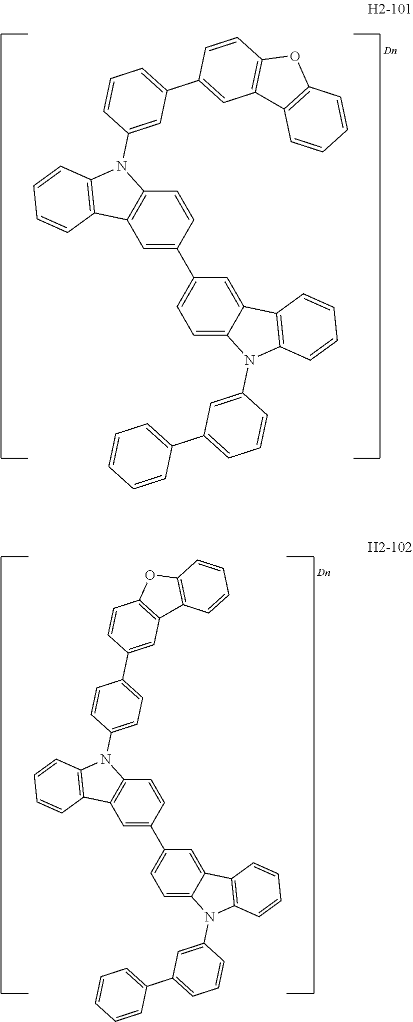

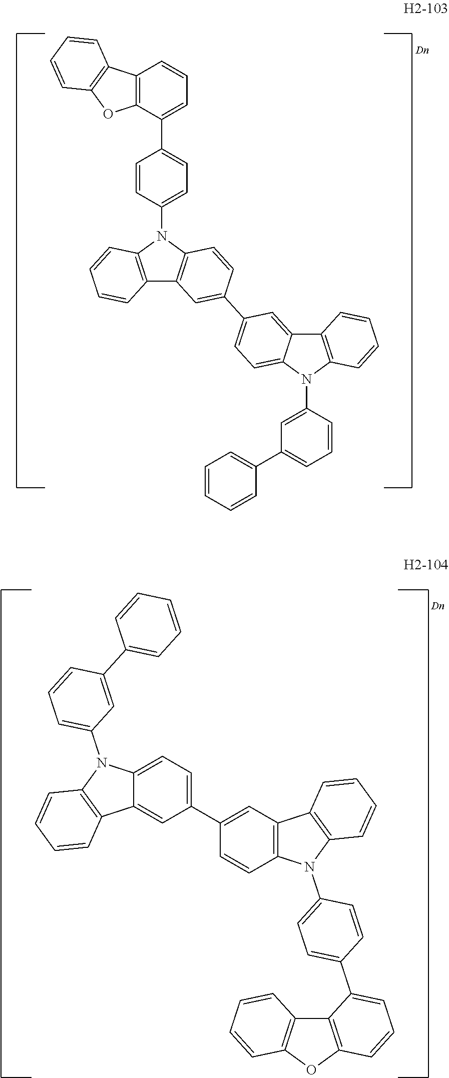

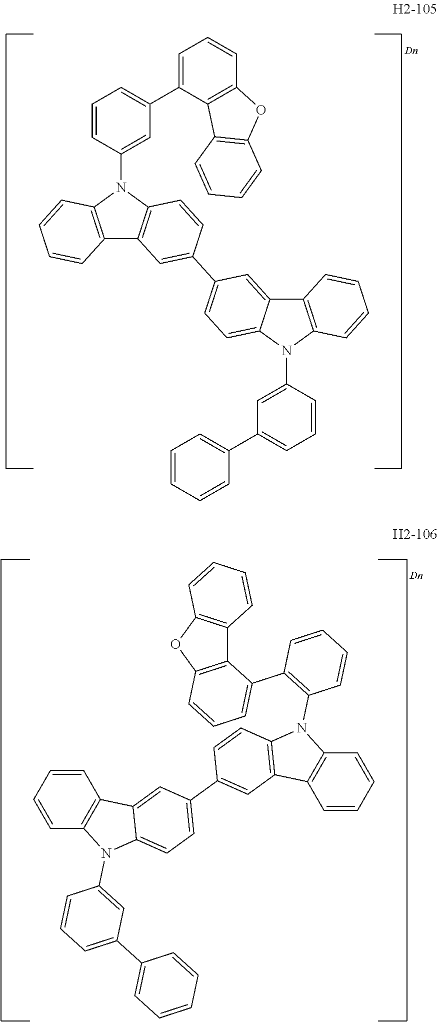

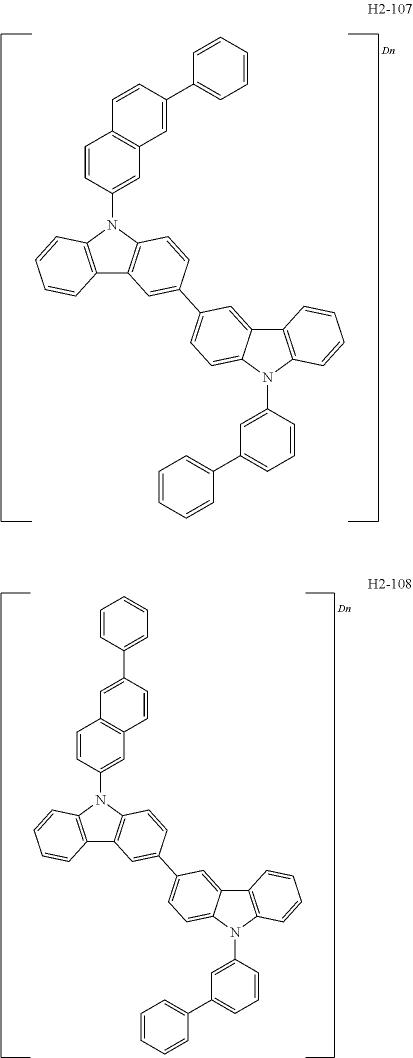

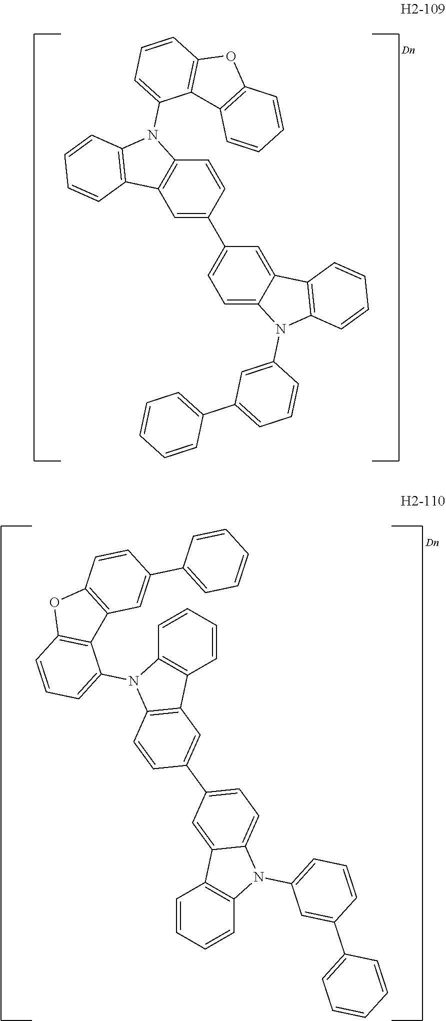

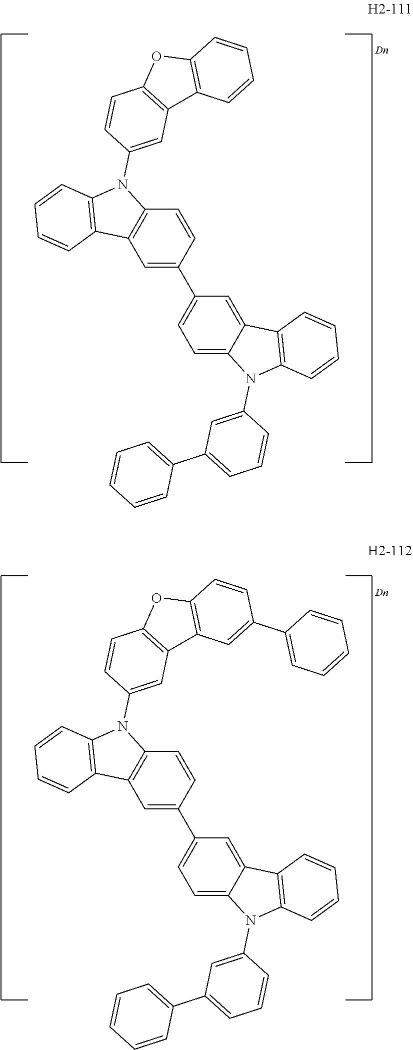

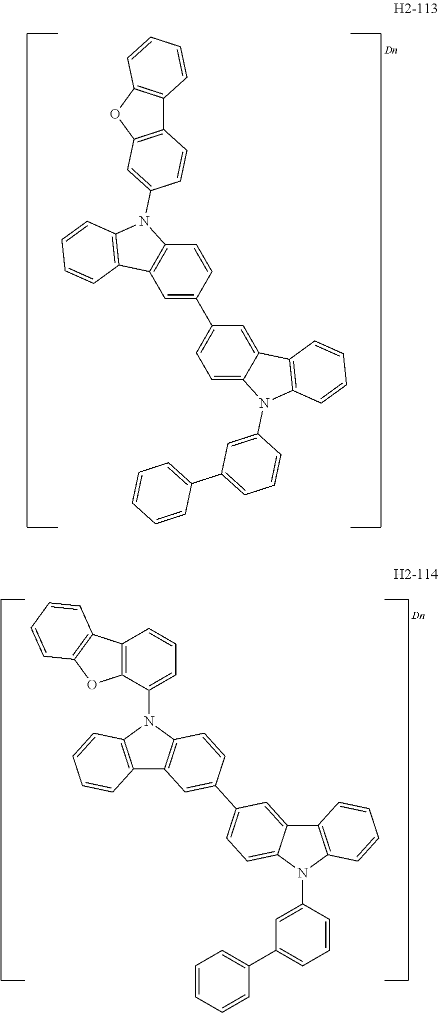

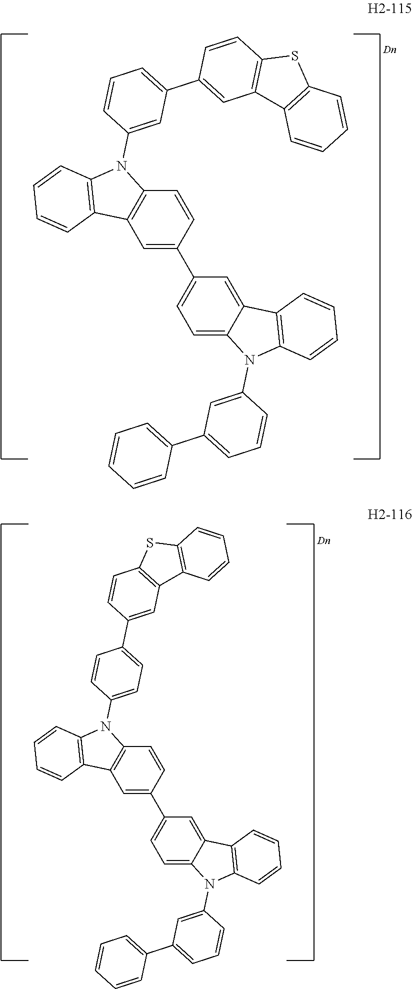

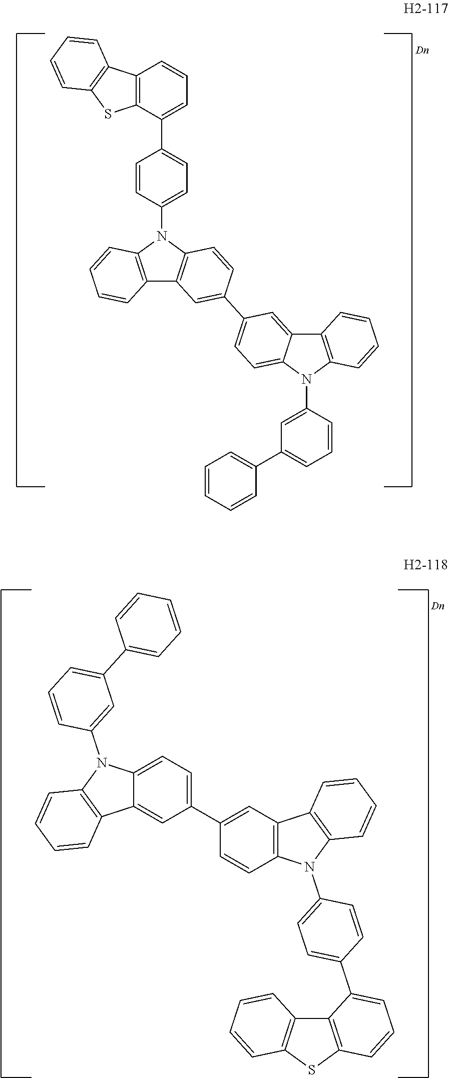

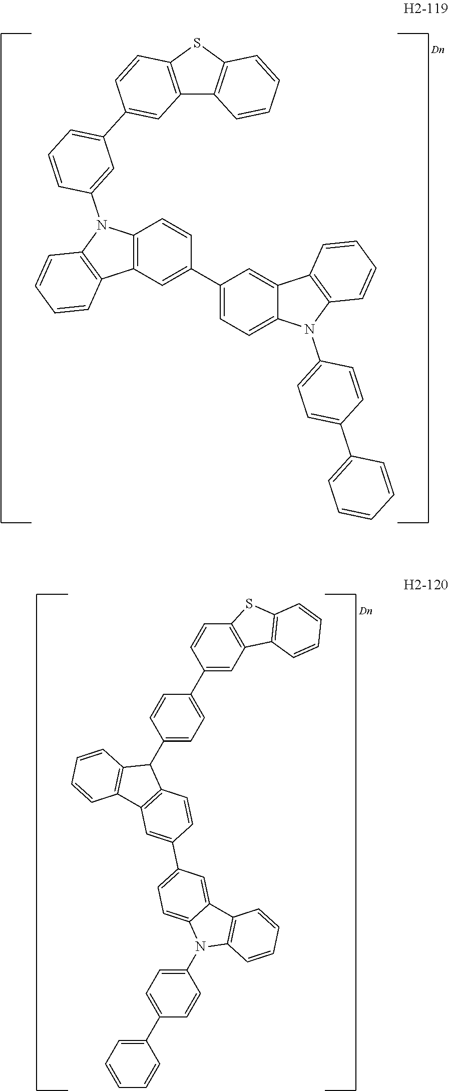

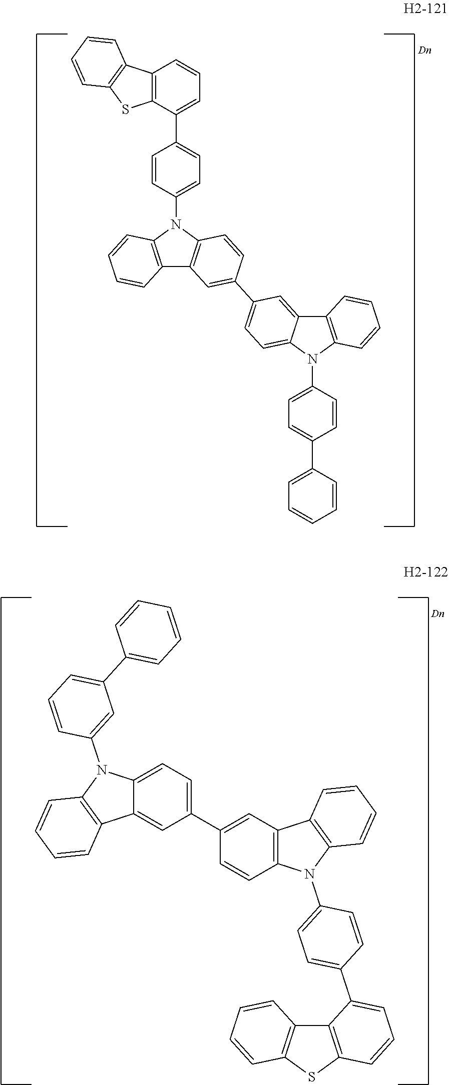

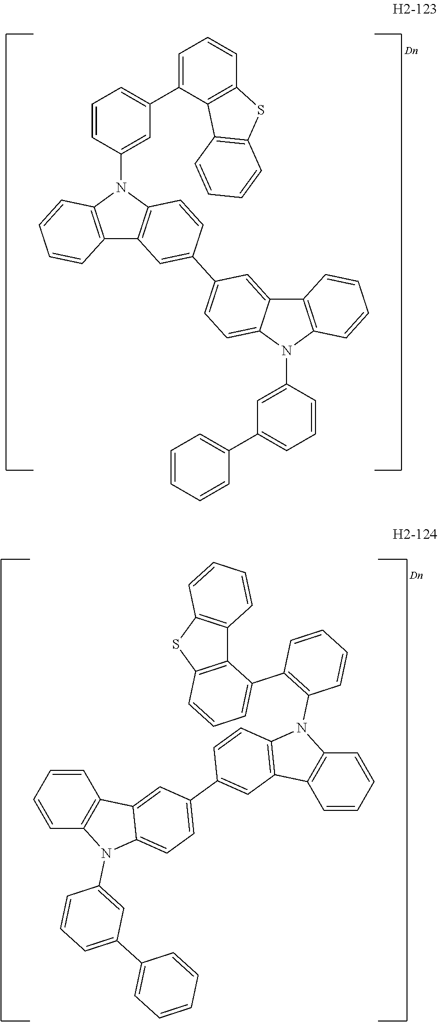

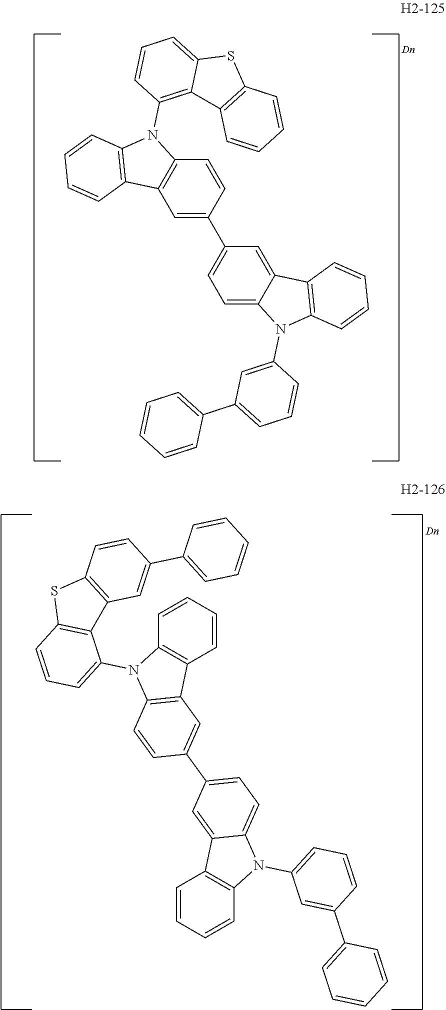

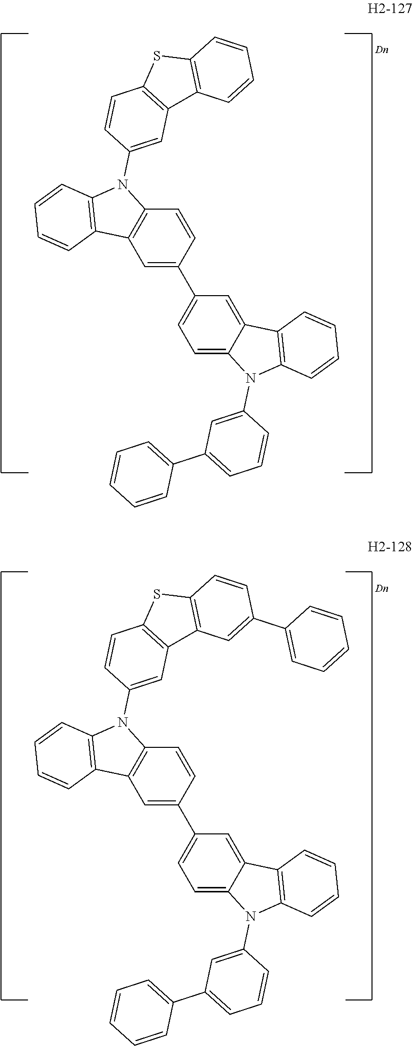

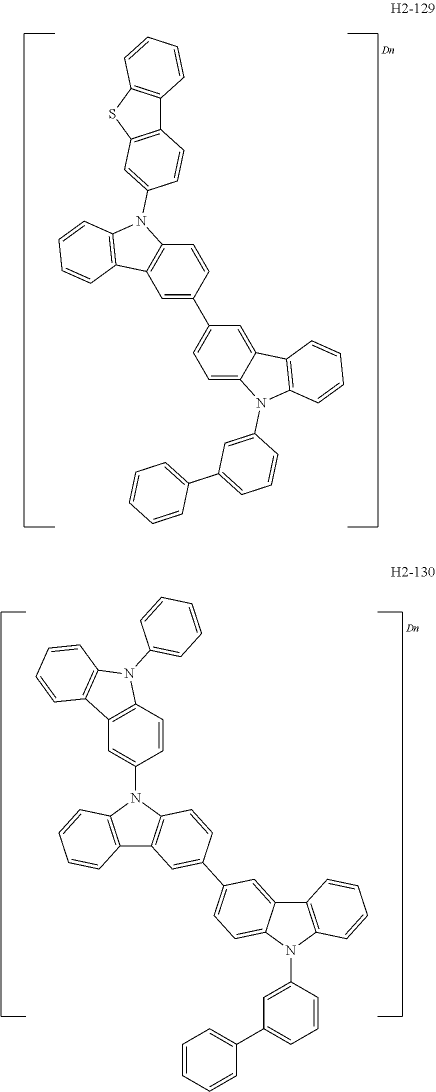

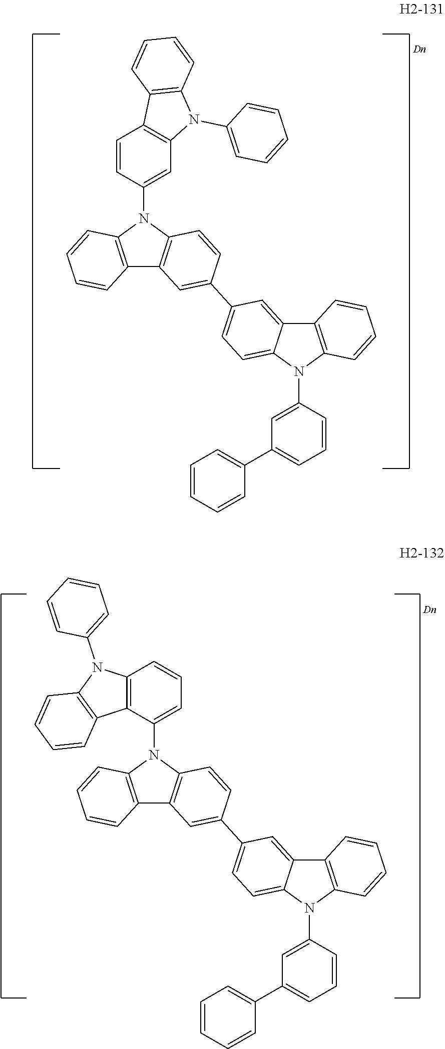

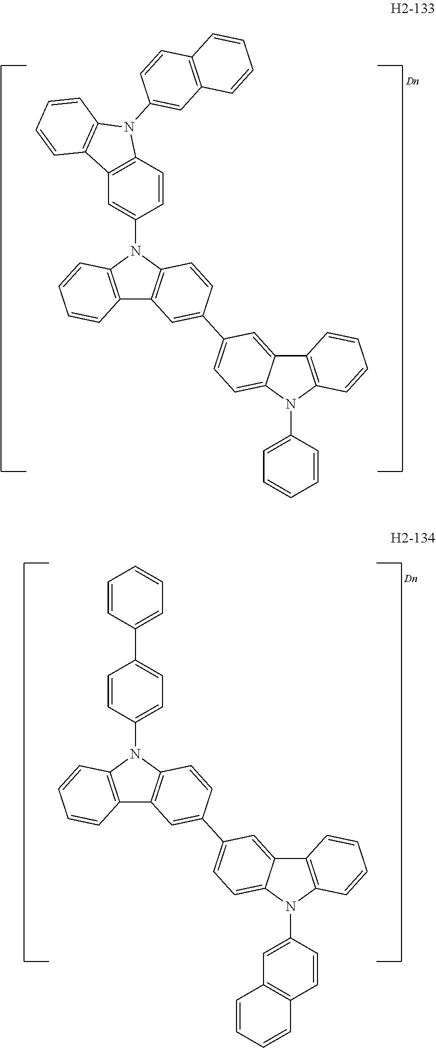

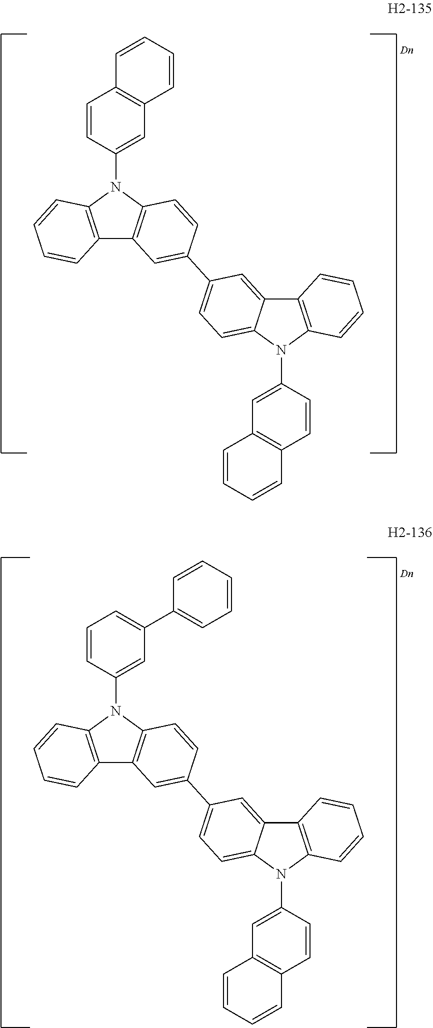

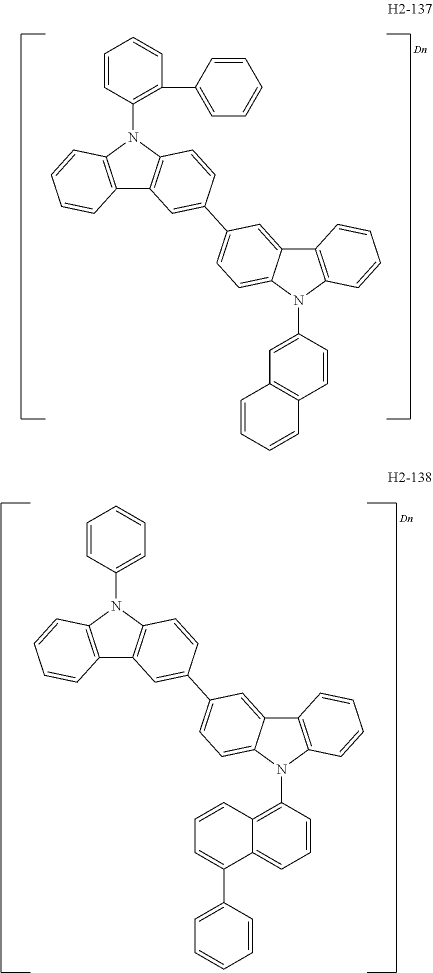

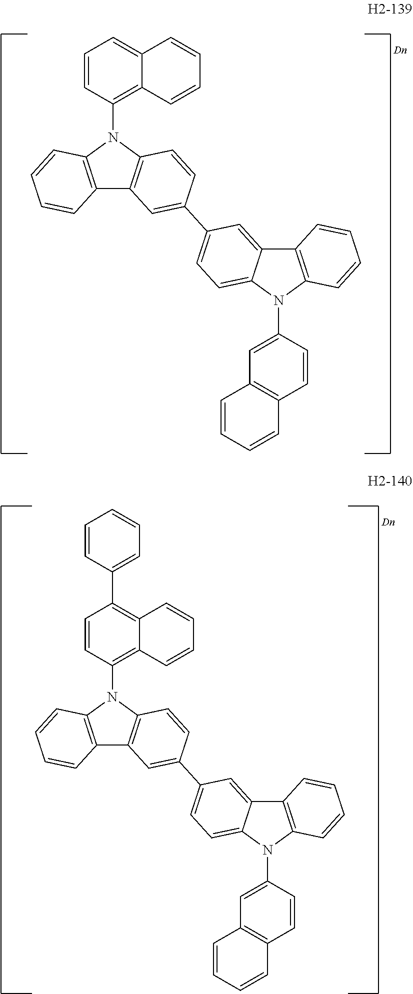

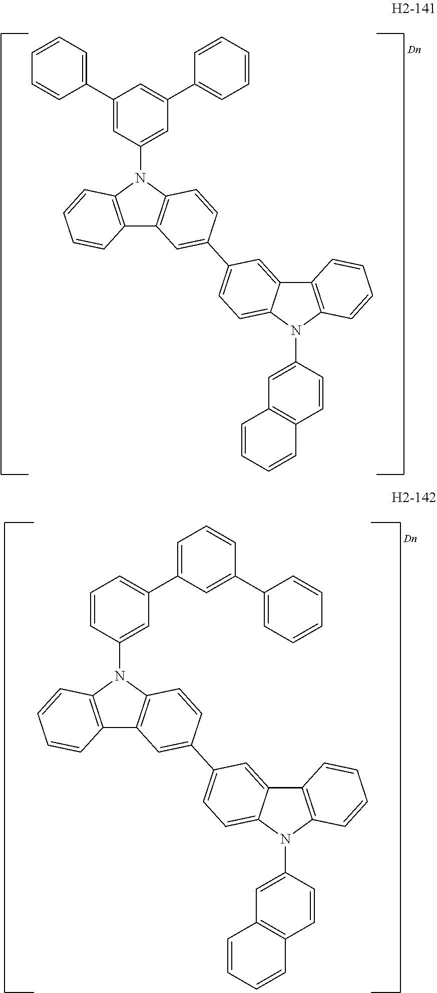

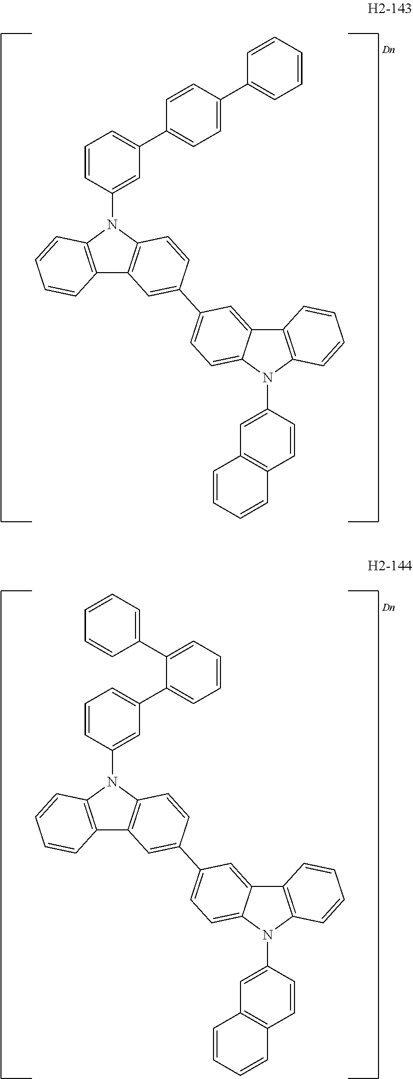

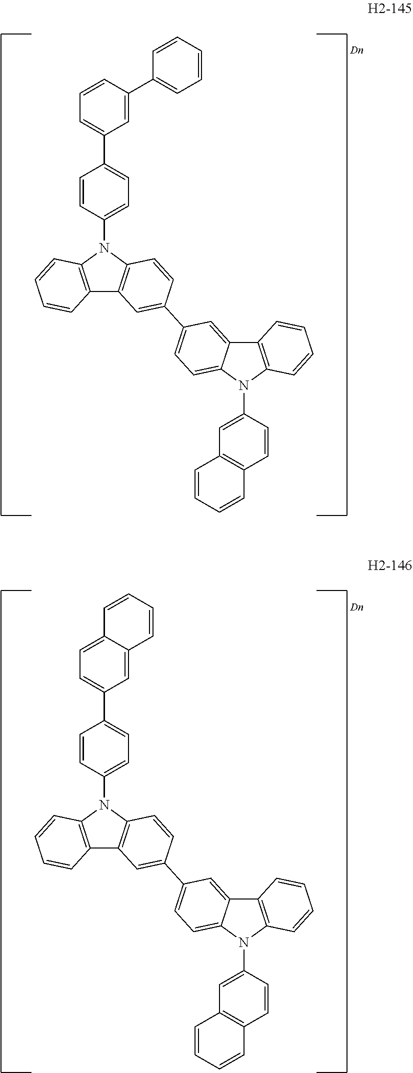

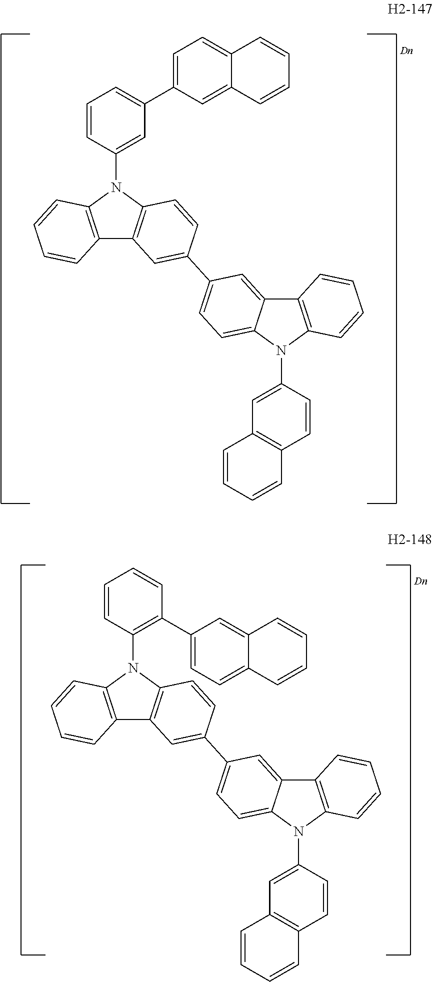

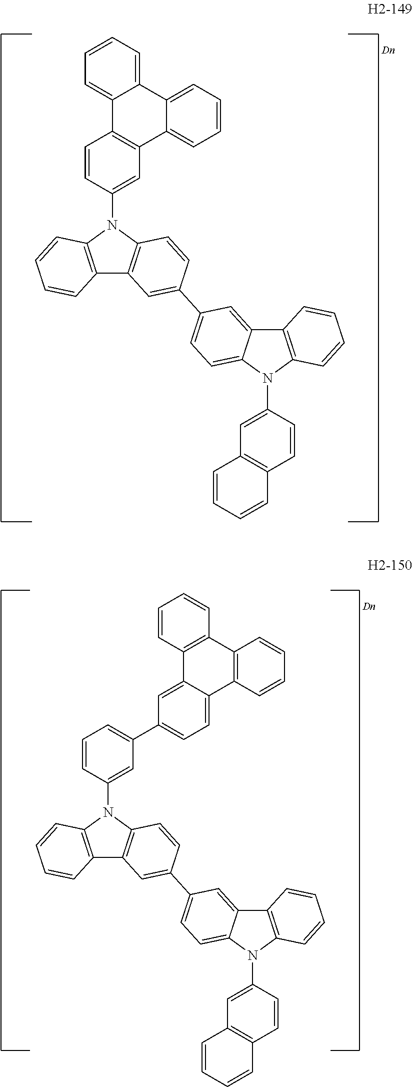

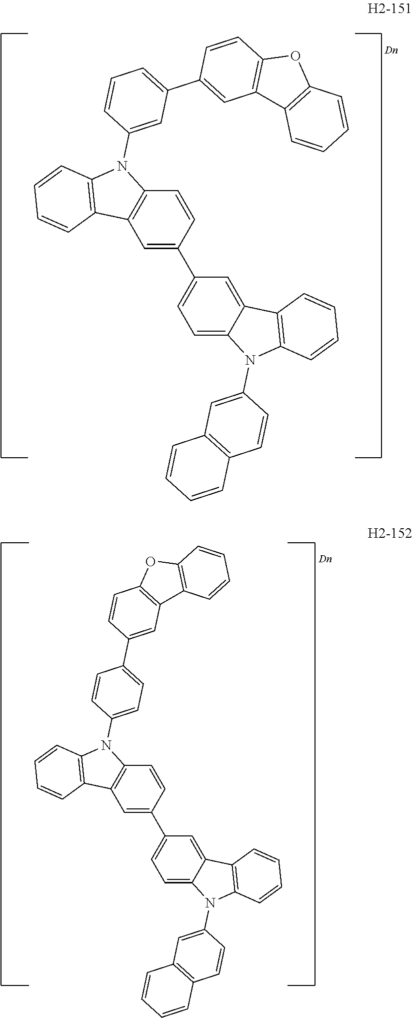

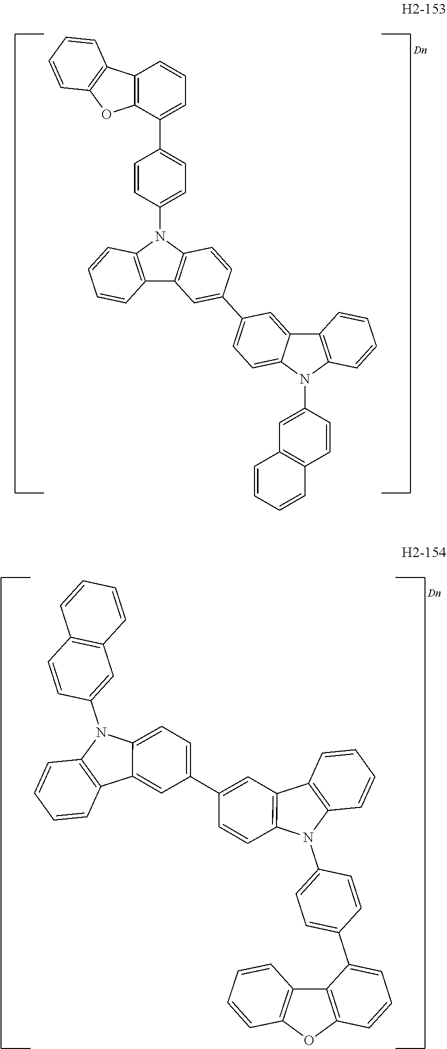

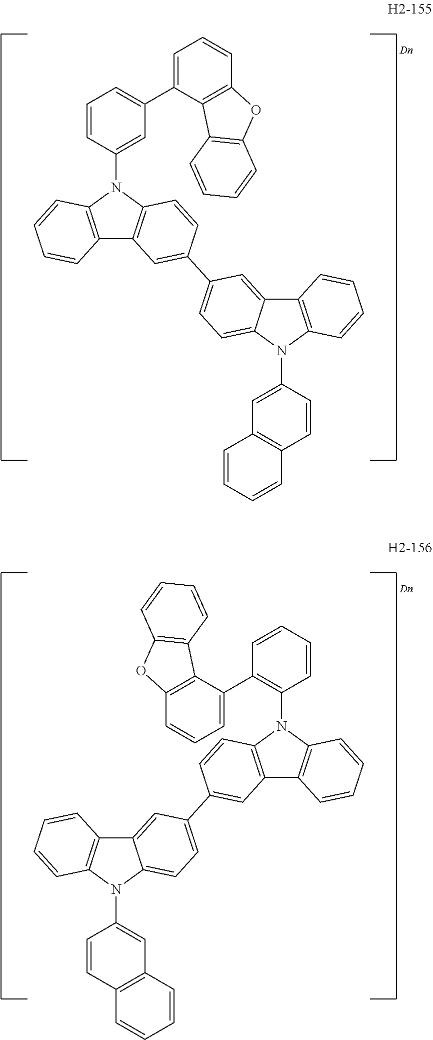

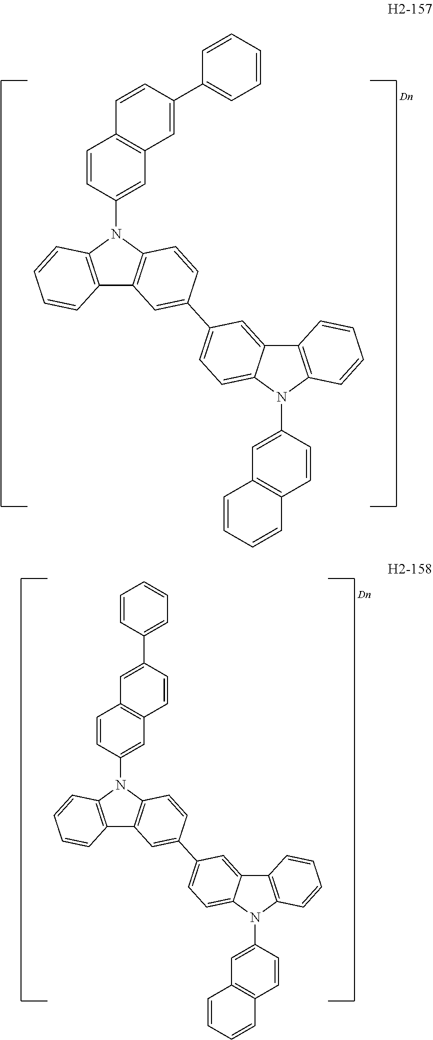

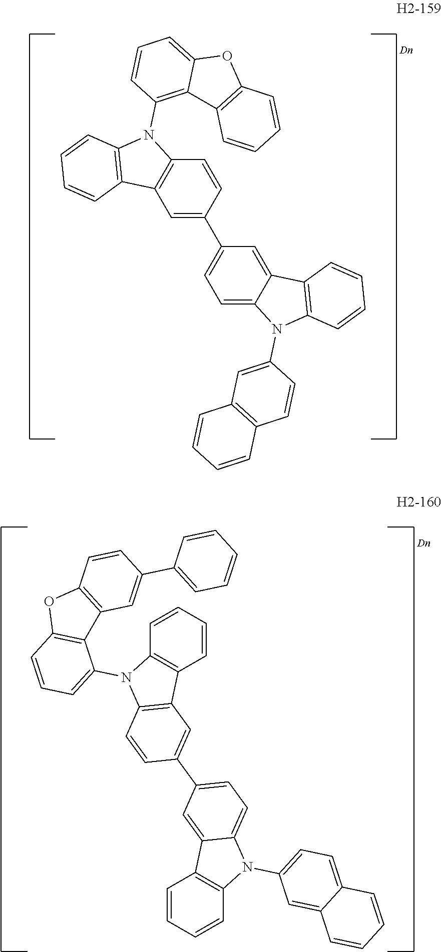

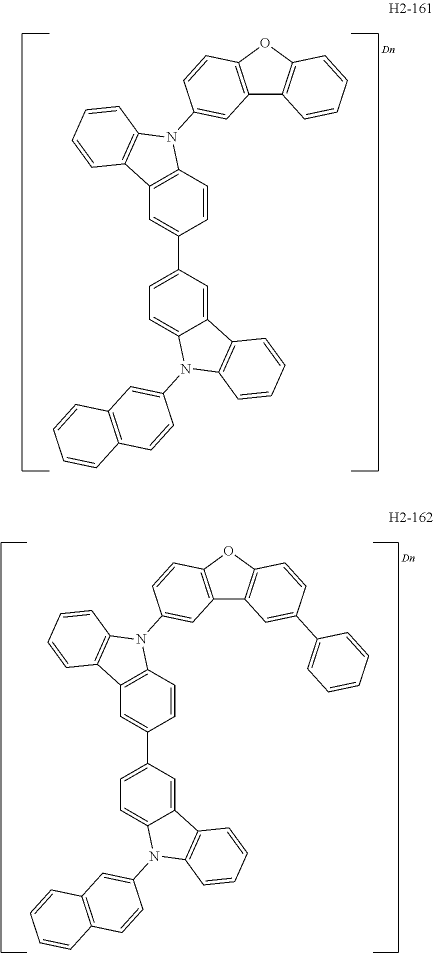

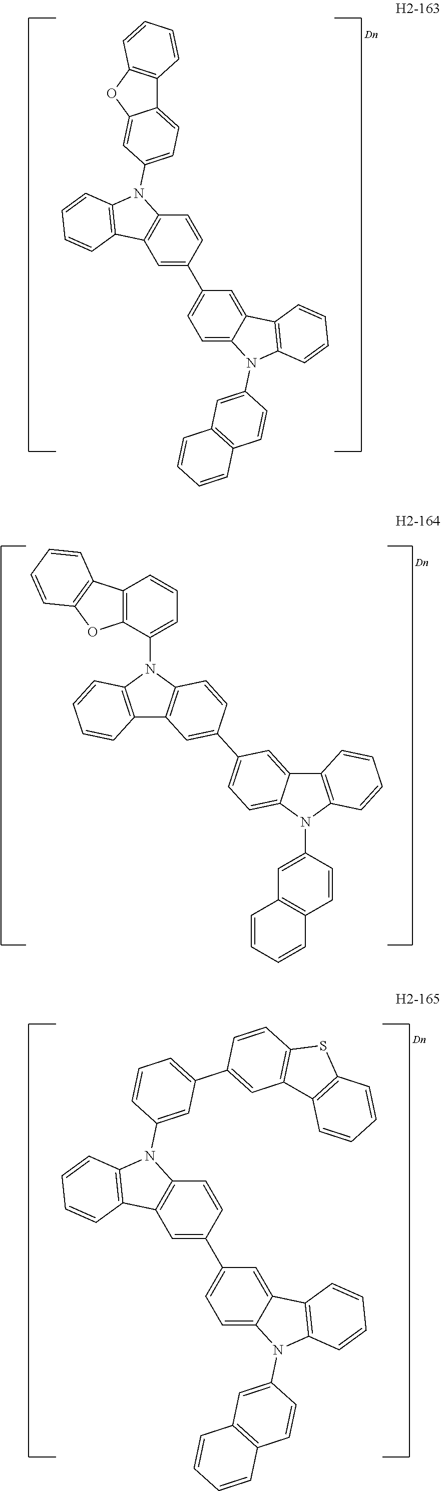

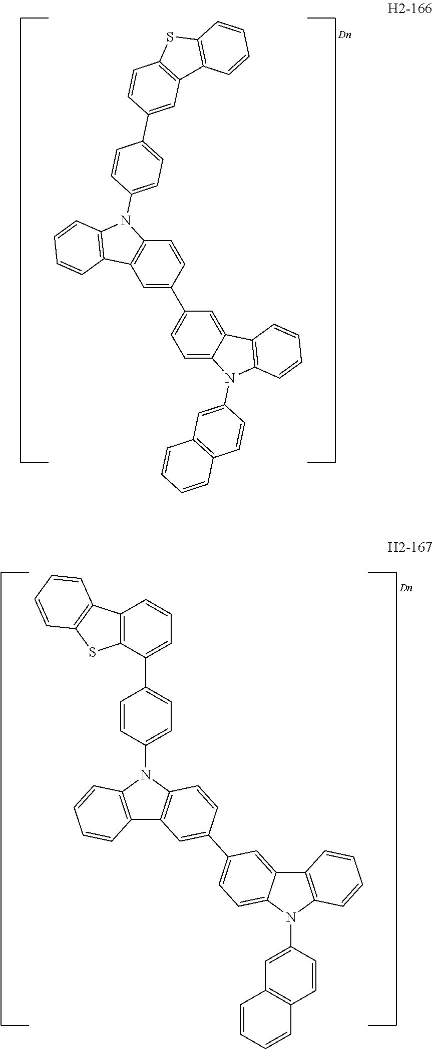

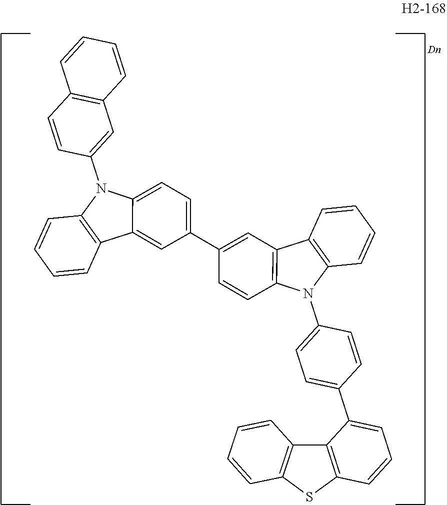

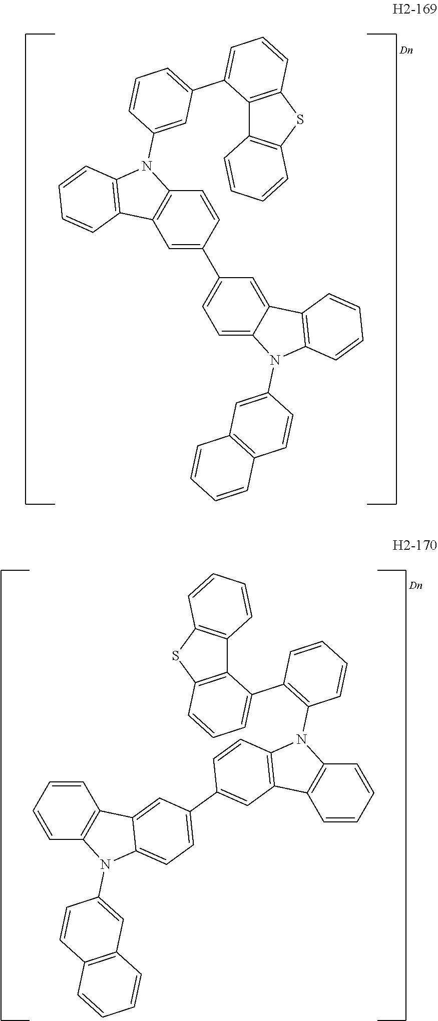

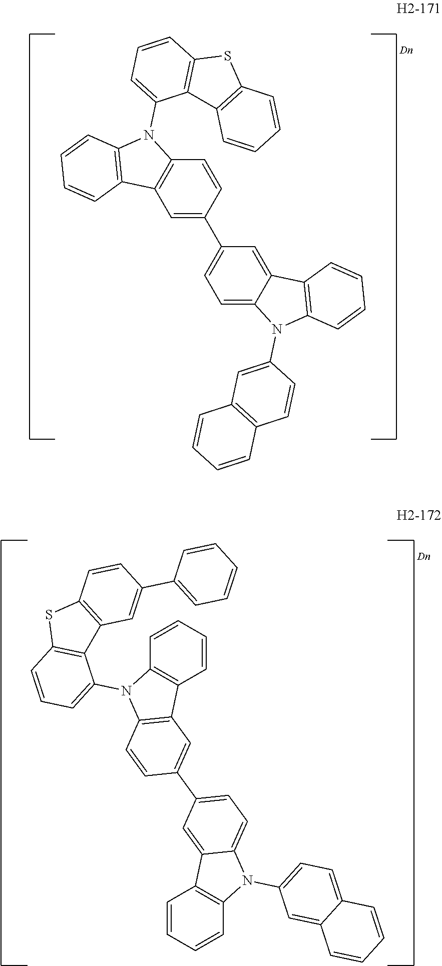

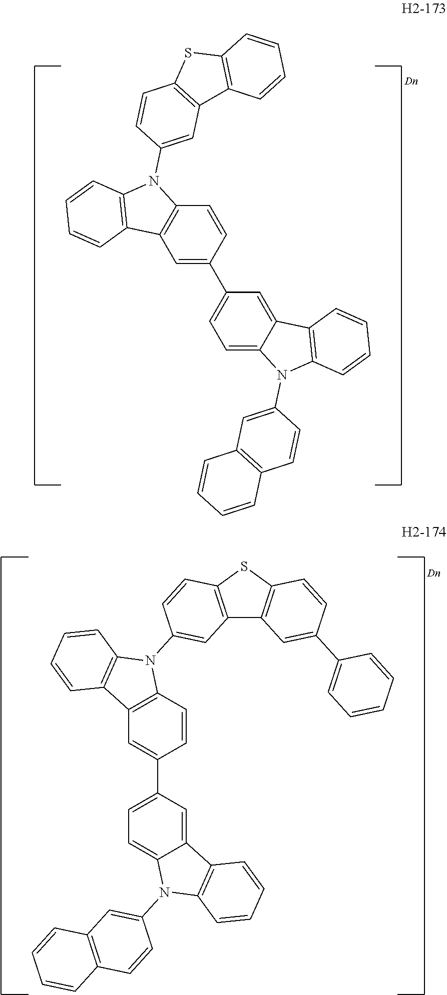

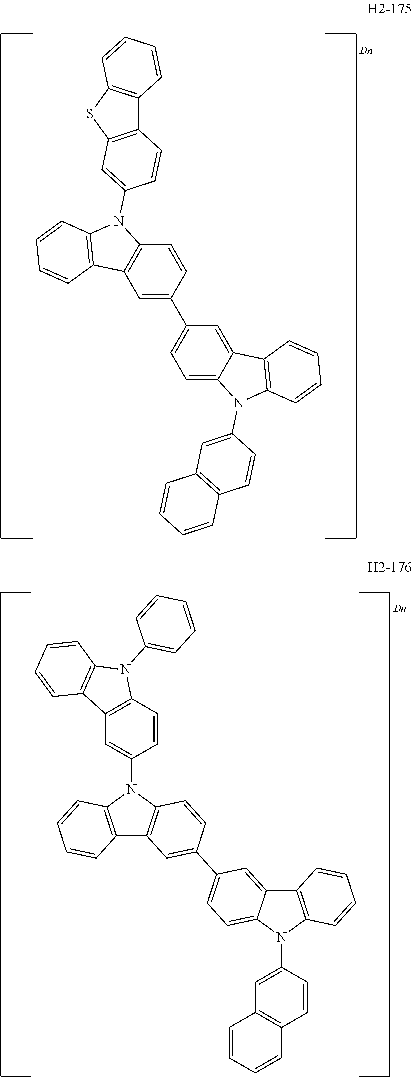

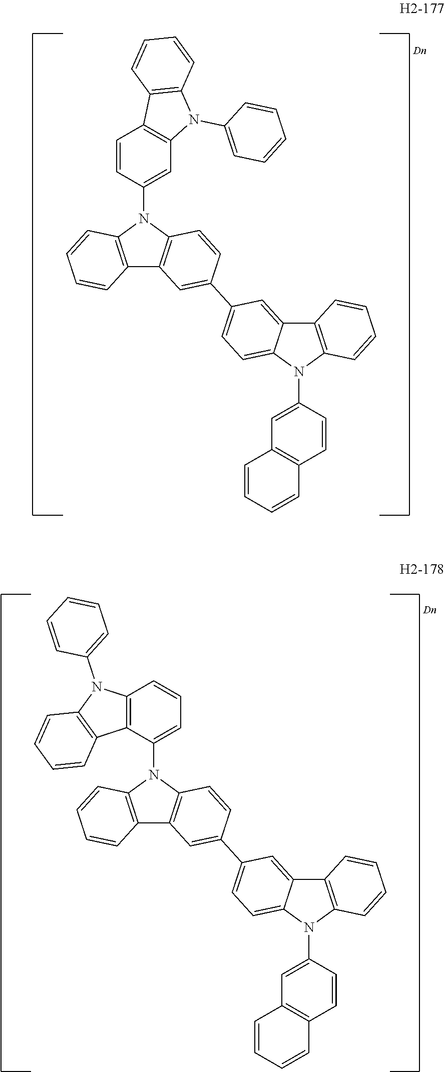

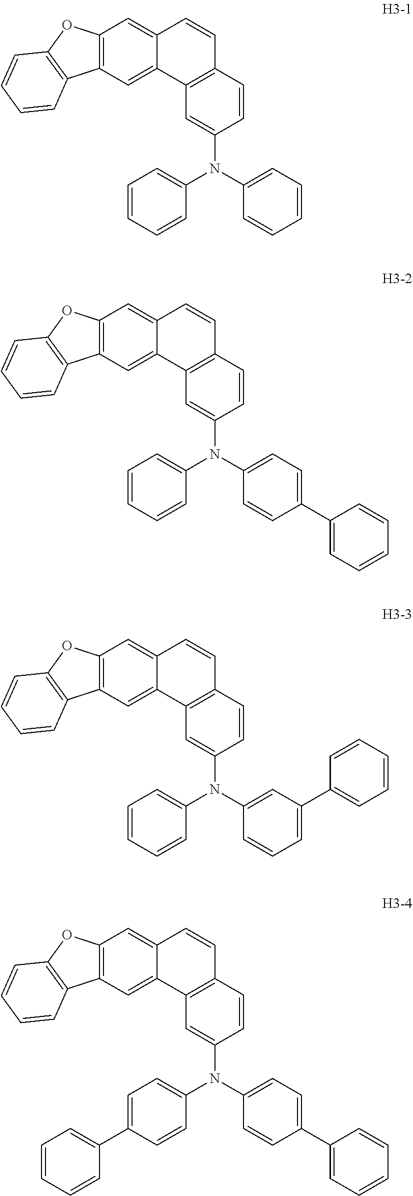 [SMILES: c1ccc(N(c2ccccc2)c2ccc3ccc4cc5oc6ccccc6c5cc4c3c2)cc1 ; c1ccc(-c2ccc(N(c3ccccc3)c3ccc4ccc5cc6oc7ccccc7c6cc5c4c3)cc2)cc1 ; c1ccc(-c2cccc(N(c3ccccc3)c3ccc4ccc5cc6oc7ccccc7c6cc5c4c3)c2)cc1 ; c1ccc(-c2ccc(N(c3ccc(-c4ccccc4)cc3)c3ccc4ccc5cc6oc7ccccc7c6cc5c4c3)cc2)cc1]

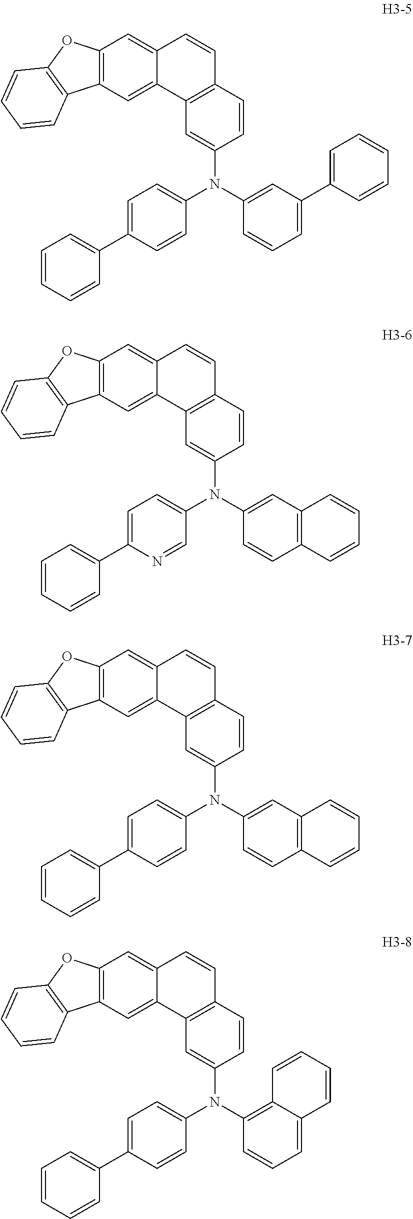

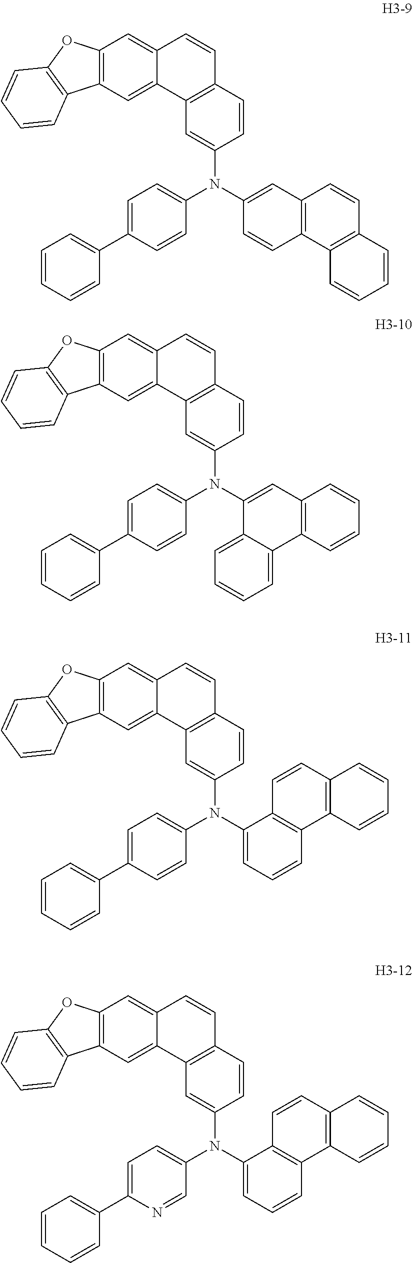

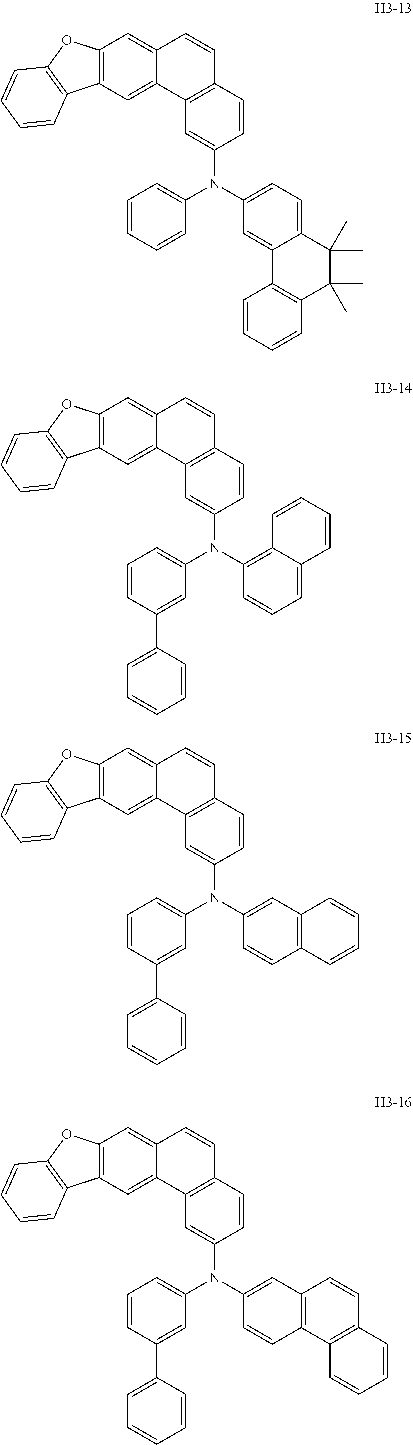

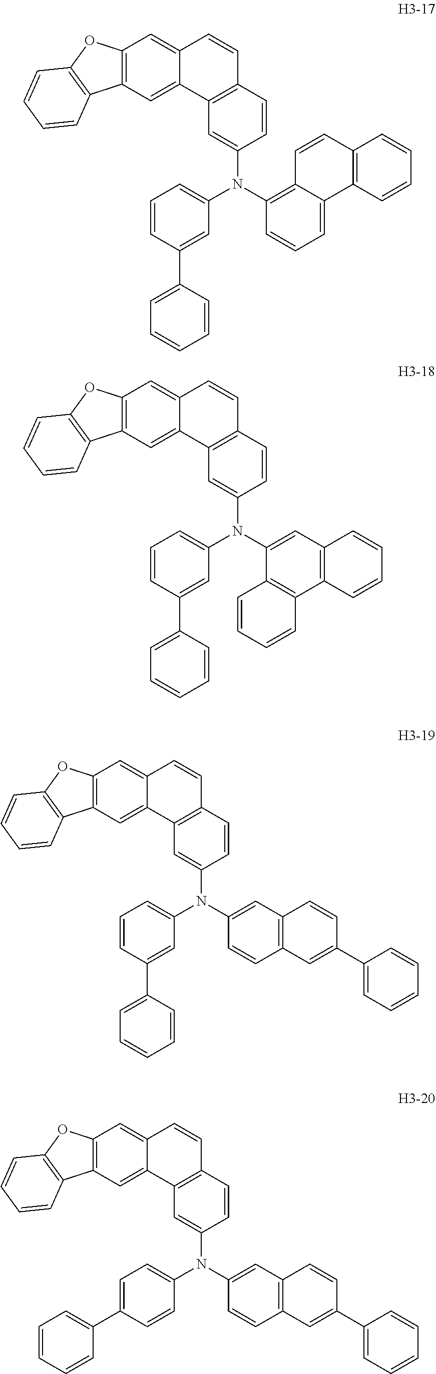

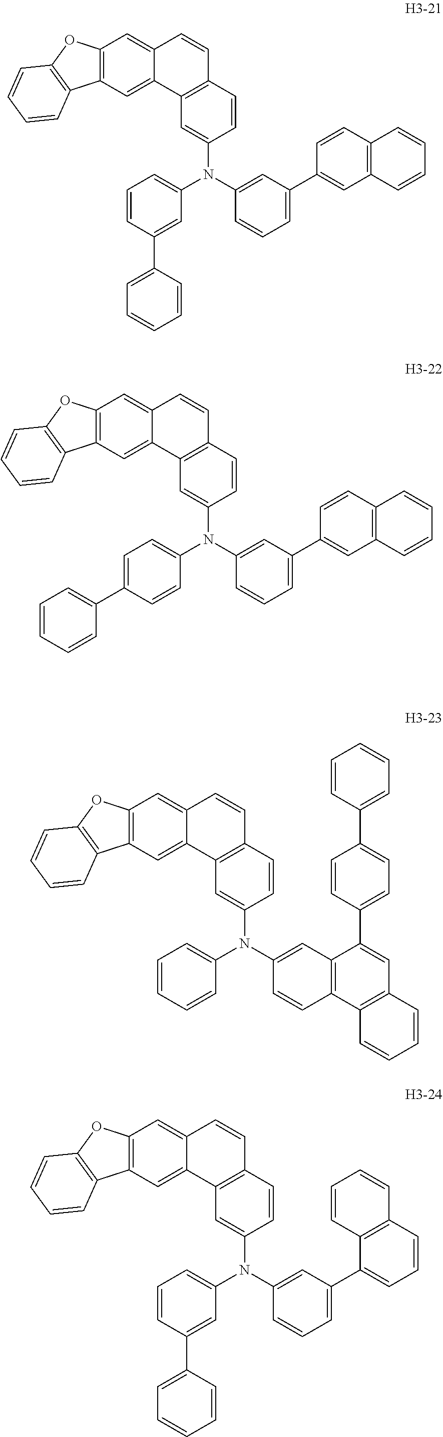 [SMILES: c1ccc(-c2cccc(N(c3cccc(-c4ccc5ccccc5c4)c3)c3ccc4ccc5cc6oc7ccccc7c6cc5c4c3)c2)cc1 ; c1ccc(-c2ccc(N(c3cccc(-c4ccc5ccccc5c4)c3)c3ccc4ccc5cc6oc7ccccc7c6cc5c4c3)cc2)cc1 ; c1ccc(-c2ccc(-c3cc4ccccc4c4ccc(N(c5ccccc5)c5ccc6ccc7cc8oc9ccccc9c8cc7c6c5)cc34)cc2)cc1 ; c1ccc(-c2cccc(N(c3cccc(-c4cccc5ccccc45)c3)c3ccc4ccc5cc6oc7ccccc7c6cc5c4c3)c2)cc1]

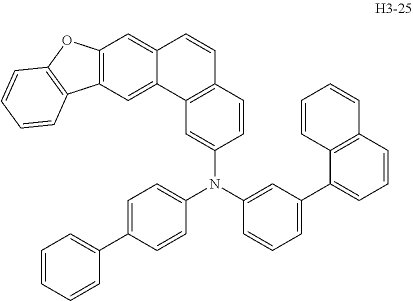 [SMILES: c1ccc(-c2ccc(N(c3cccc(-c4cccc5ccccc45)c3)c3ccc4ccc5cc6oc7ccccc7c6cc5c4c3)cc2)cc1]

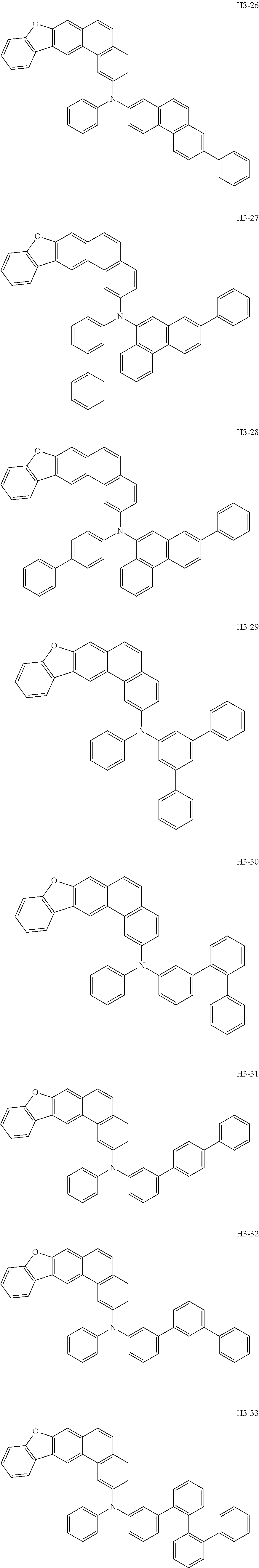

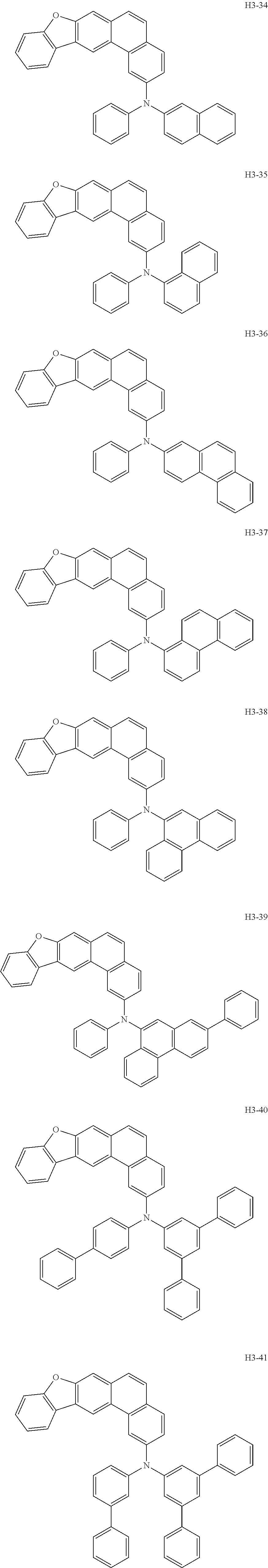

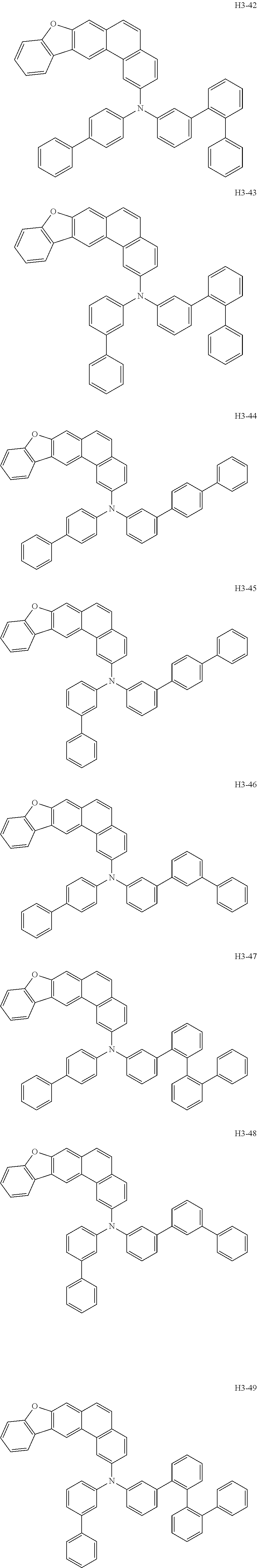

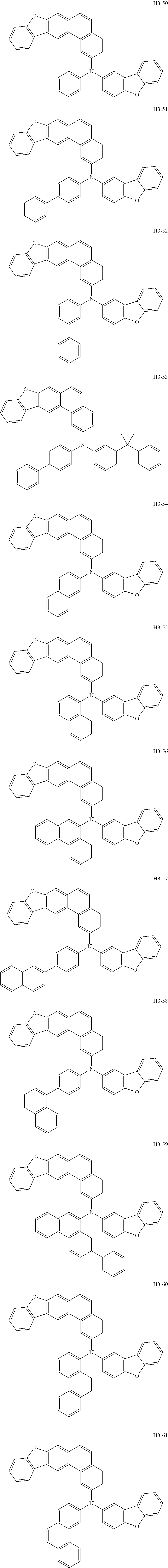

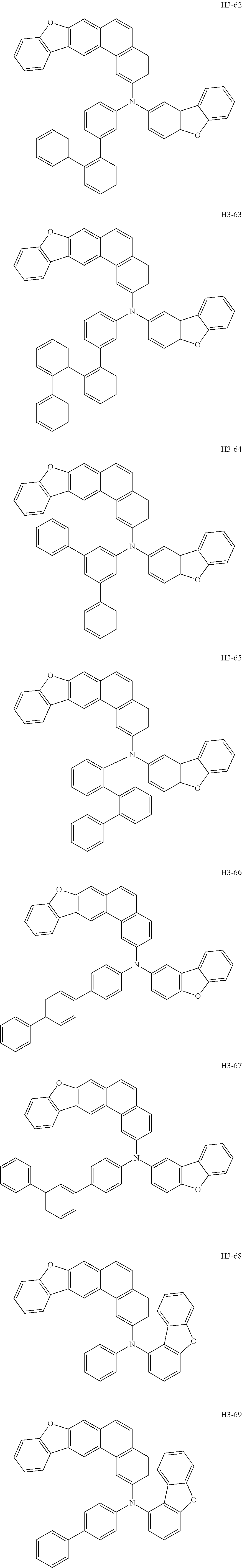

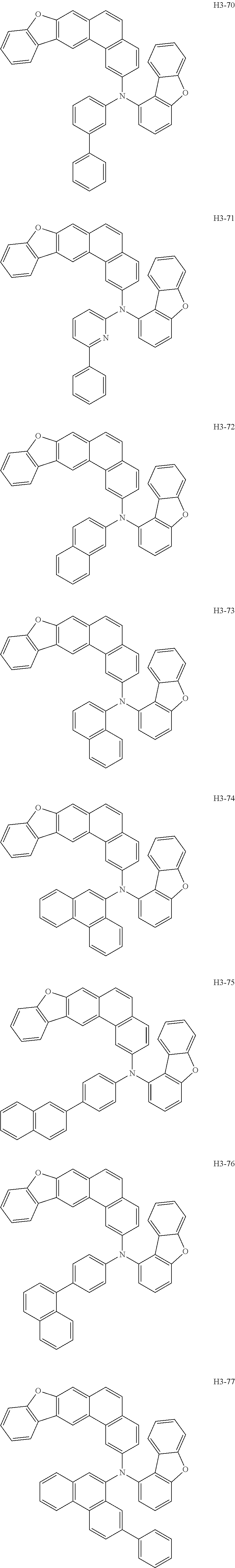

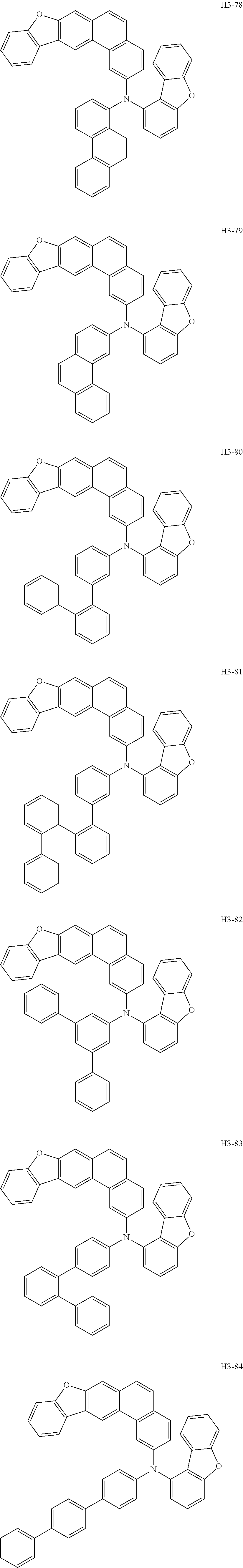

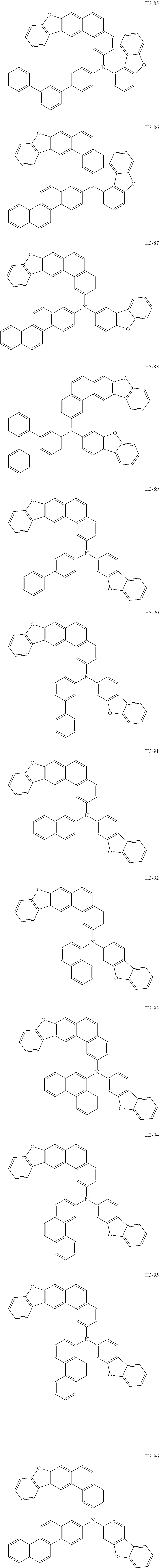

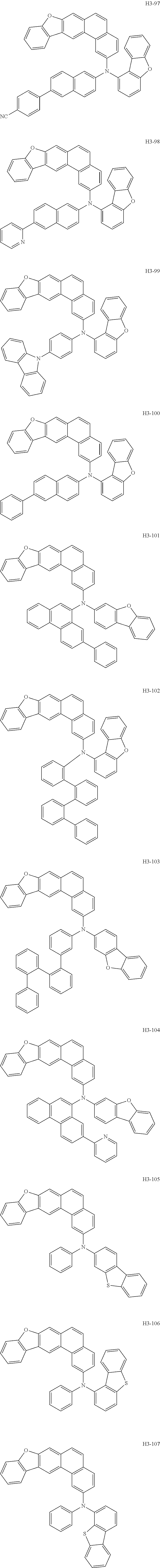

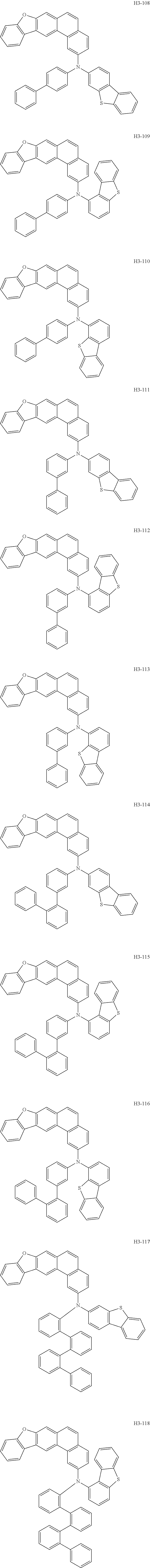

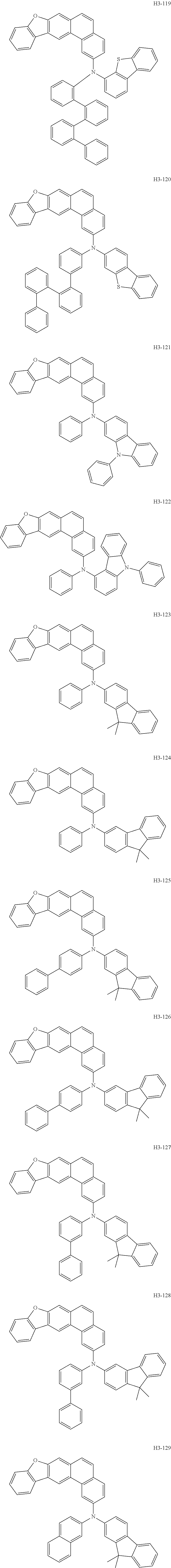

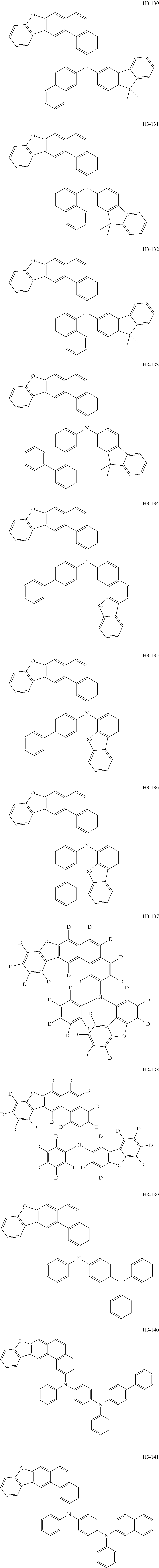

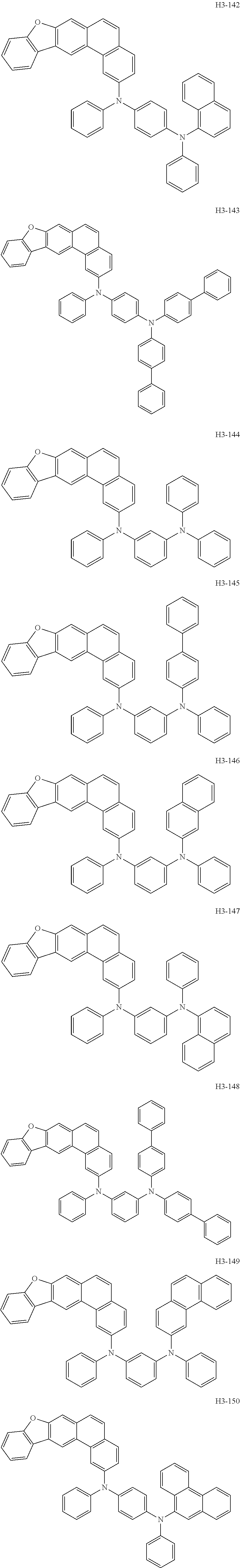

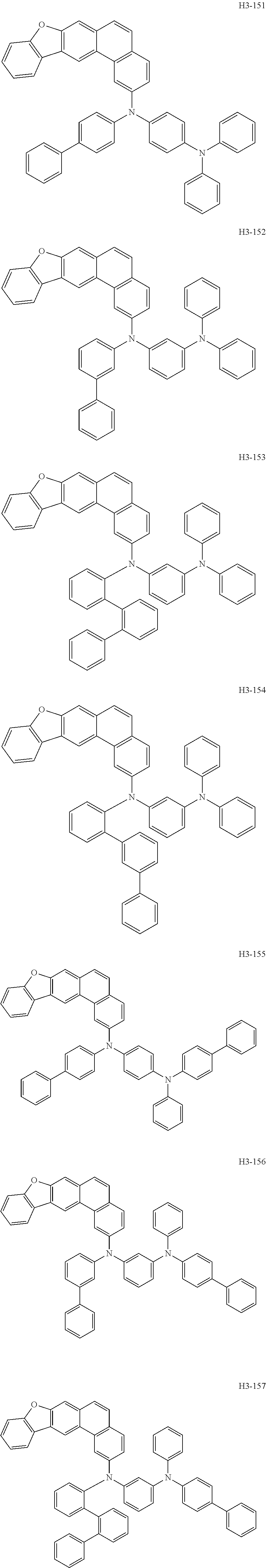

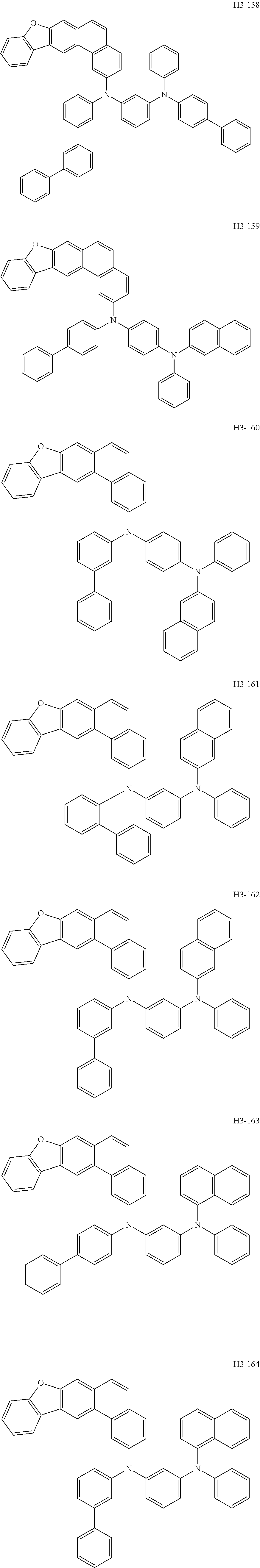

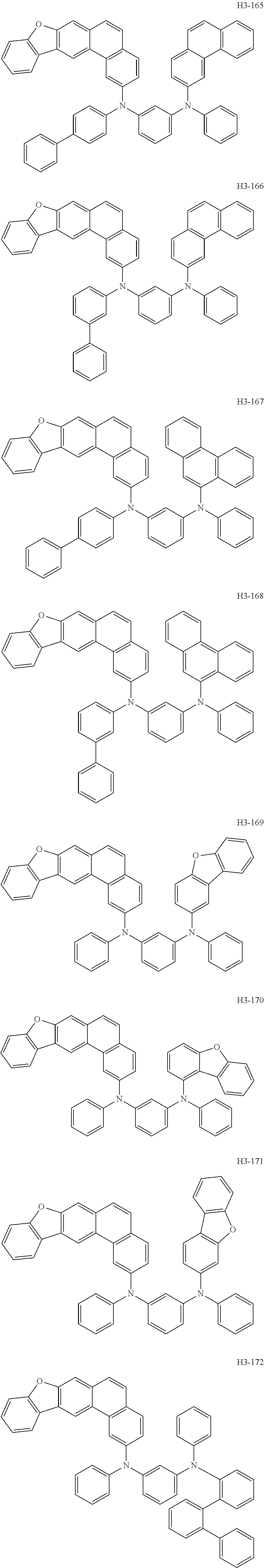

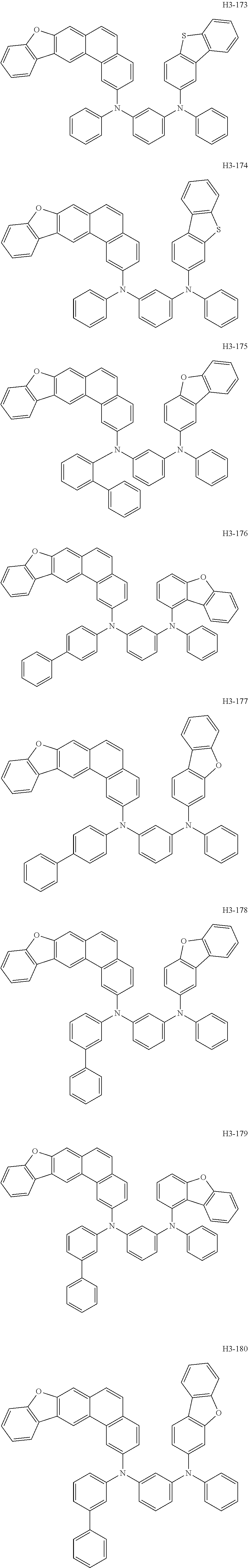

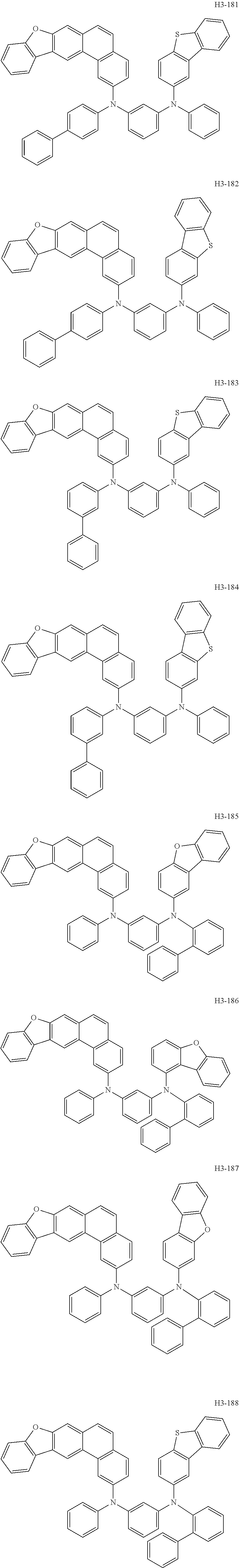

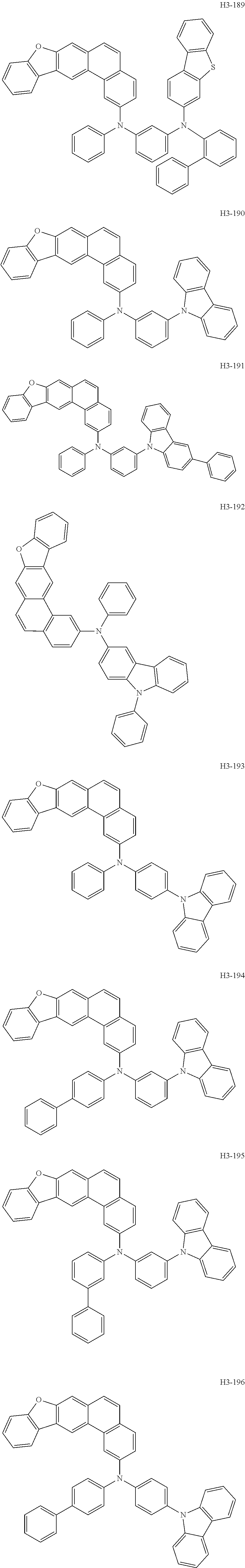

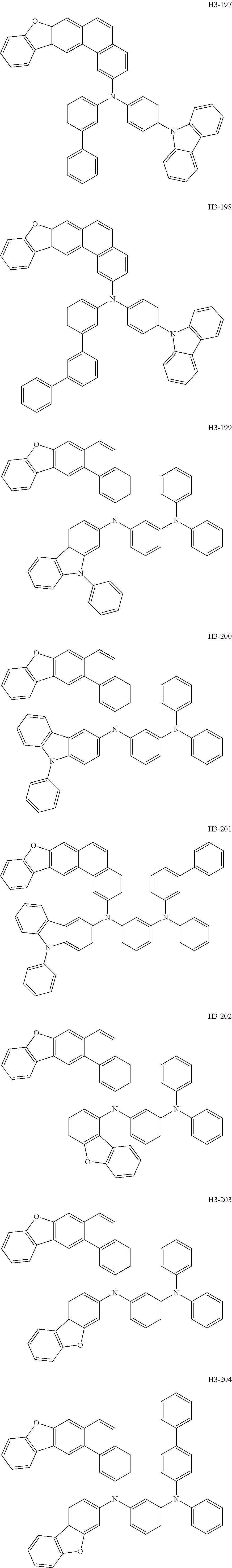

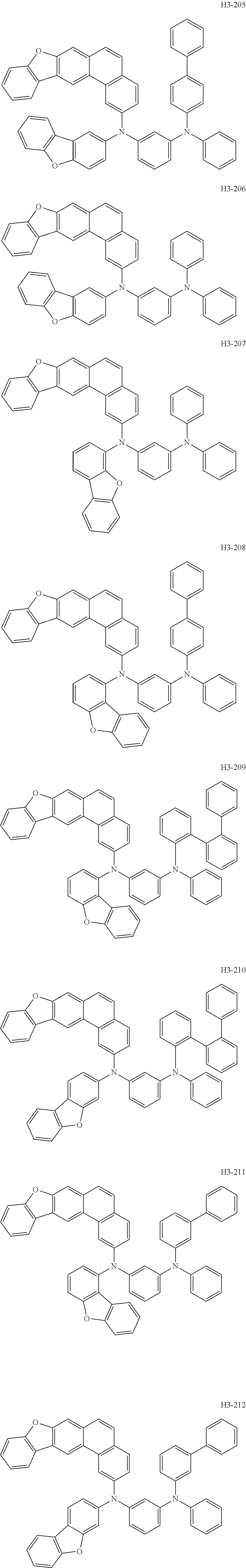

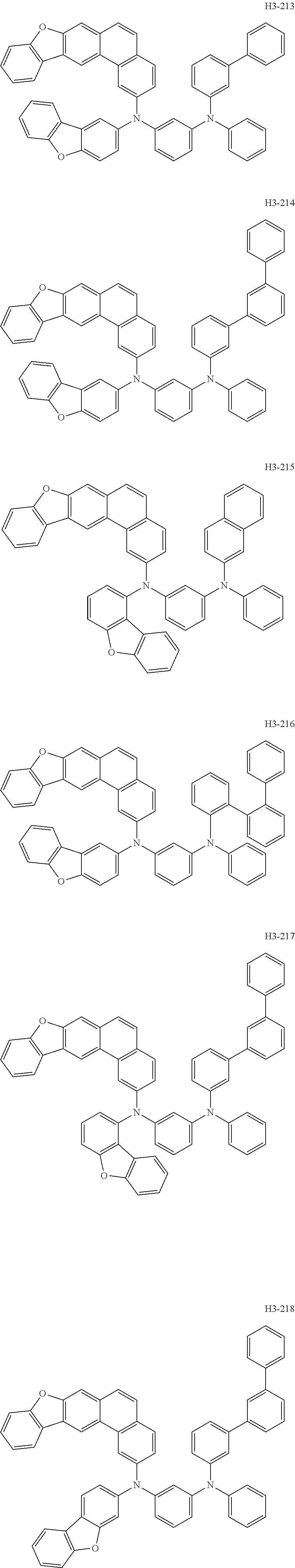

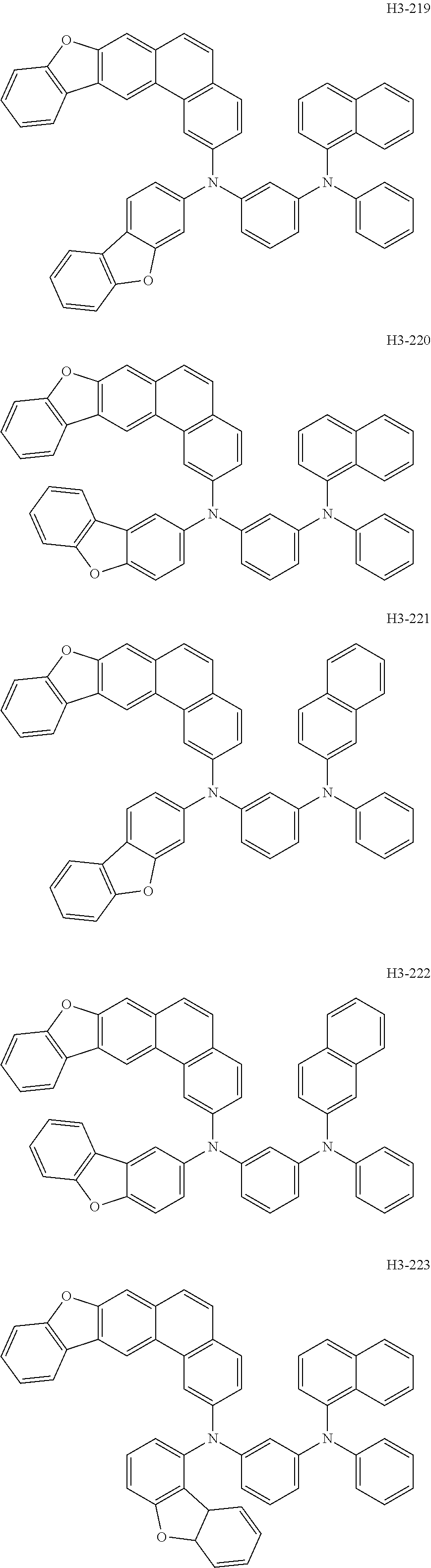 [SMILES: c1ccc(N(c2cccc(N(c3ccc4c(c3)oc3ccccc34)c3ccc4ccc5cc6oc7ccccc7c6cc5c4c3)c2)c2cccc3ccccc23)cc1 ; c1ccc(N(c2cccc(N(c3ccc4ccc5cc6oc7ccccc7c6cc5c4c3)c3ccc4oc5ccccc5c4c3)c2)c2cccc3ccccc23)cc1 ; c1ccc(N(c2cccc(N(c3ccc4c(c3)oc3ccccc34)c3ccc4ccc5cc6oc7ccccc7c6cc5c4c3)c2)c2ccc3ccccc3c2)cc1 ; c1ccc(N(c2cccc(N(c3ccc4ccc5cc6oc7ccccc7c6cc5c4c3)c3ccc4oc5ccccc5c4c3)c2)c2ccc3ccccc3c2)cc1 ; C1=CC2Oc3cccc(N(c4cccc(N(c5ccccc5)c5cccc6ccccc56)c4)c4ccc5ccc6cc7oc8ccccc8c7cc6c5c4)c3C2C=C1]

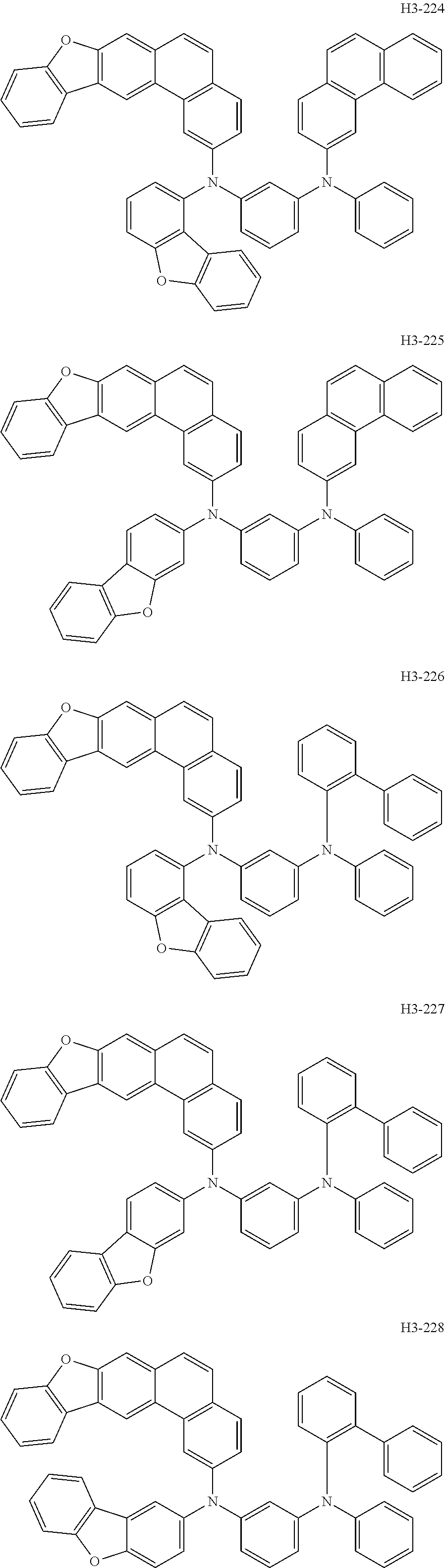 [SMILES: c1ccc(N(c2cccc(N(c3ccc4ccc5cc6oc7ccccc7c6cc5c4c3)c3cccc4oc5ccccc5c34)c2)c2ccc3ccc4ccccc4c3c2)cc1 ; c1ccc(N(c2cccc(N(c3ccc4c(c3)oc3ccccc34)c3ccc4ccc5cc6oc7ccccc7c6cc5c4c3)c2)c2ccc3ccc4ccccc4c3c2)cc1 ; c1ccc(-c2ccccc2N(c2ccccc2)c2cccc(N(c3ccc4ccc5cc6oc7ccccc7c6cc5c4c3)c3cccc4oc5ccccc5c34)c2)cc1 ; c1ccc(-c2ccccc2N(c2ccccc2)c2cccc(N(c3ccc4c(c3)oc3ccccc34)c3ccc4ccc5cc6oc7ccccc7c6cc5c4c3)c2)cc1 ; c1ccc(-c2ccccc2N(c2ccccc2)c2cccc(N(c3ccc4ccc5cc6oc7ccccc7c6cc5c4c3)c3ccc4oc5ccccc5c4c3)c2)cc1]

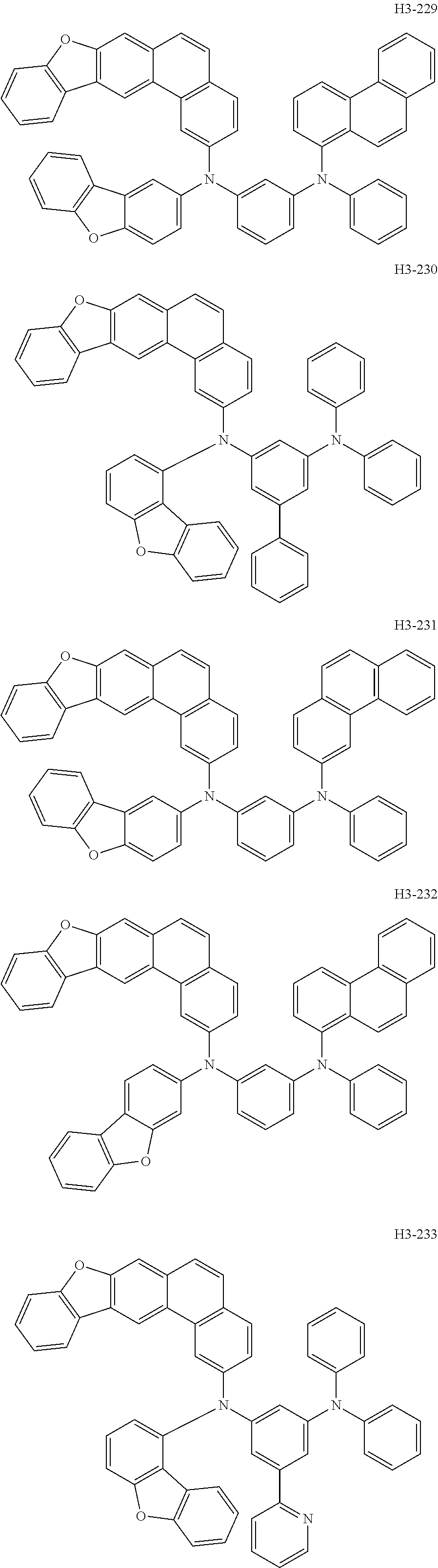 [SMILES: c1ccc(N(c2cccc(N(c3ccc4ccc5cc6oc7ccccc7c6cc5c4c3)c3ccc4oc5ccccc5c4c3)c2)c2cccc3c2ccc2ccccc23)cc1 ; c1ccc(-c2cc(N(c3ccccc3)c3ccccc3)cc(N(c3ccc4ccc5cc6oc7ccccc7c6cc5c4c3)c3cccc4oc5ccccc5c34)c2)cc1 ; c1ccc(N(c2cccc(N(c3ccc4ccc5cc6oc7ccccc7c6cc5c4c3)c3ccc4oc5ccccc5c4c3)c2)c2ccc3ccc4ccccc4c3c2)cc1 ; c1ccc(N(c2cccc(N(c3ccc4c(c3)oc3ccccc34)c3ccc4ccc5cc6oc7ccccc7c6cc5c4c3)c2)c2cccc3c2ccc2ccccc23)cc1 ; c1ccc(N(c2ccccc2)c2cc(-c3ccccn3)cc(N(c3ccc4ccc5cc6oc7ccccc7c6cc5c4c3)c3cccc4oc5ccccc5c34)c2)cc1]

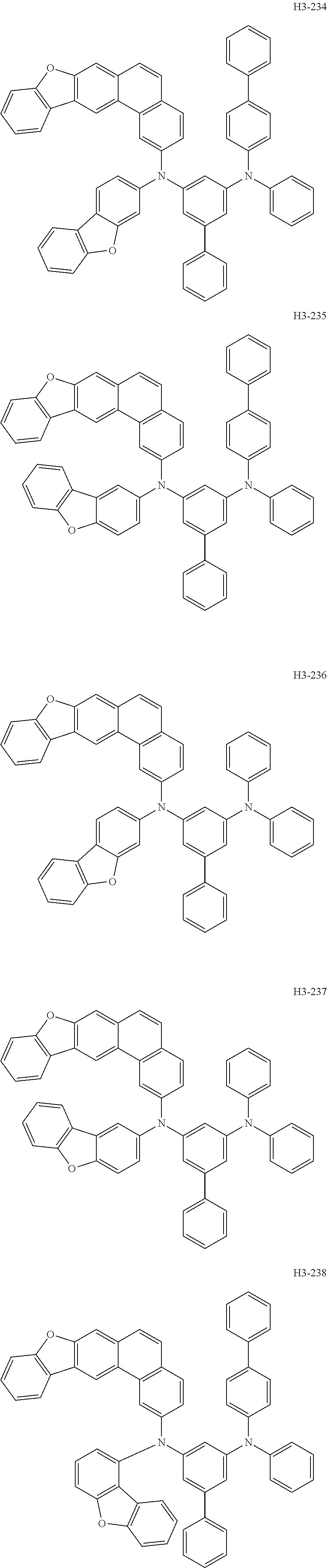

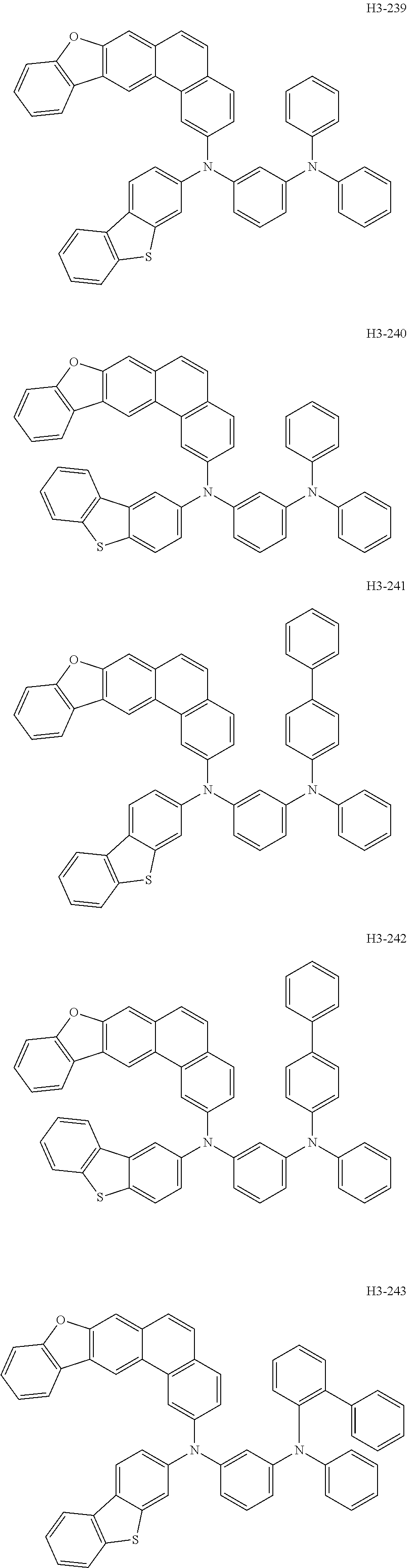 [SMILES: c1ccc(N(c2ccccc2)c2cccc(N(c3ccc4c(c3)sc3ccccc34)c3ccc4ccc5cc6oc7ccccc7c6cc5c4c3)c2)cc1 ; c1ccc(N(c2ccccc2)c2cccc(N(c3ccc4ccc5cc6oc7ccccc7c6cc5c4c3)c3ccc4sc5ccccc5c4c3)c2)cc1 ; c1ccc(-c2ccc(N(c3ccccc3)c3cccc(N(c4ccc5c(c4)sc4ccccc45)c4ccc5ccc6cc7oc8ccccc8c7cc6c5c4)c3)cc2)cc1 ; c1ccc(-c2ccc(N(c3ccccc3)c3cccc(N(c4ccc5ccc6cc7oc8ccccc8c7cc6c5c4)c4ccc5sc6ccccc6c5c4)c3)cc2)cc1 ; c1ccc(-c2ccccc2N(c2ccccc2)c2cccc(N(c3ccc4c(c3)sc3ccccc34)c3ccc4ccc5cc6oc7ccccc7c6cc5c4c3)c2)cc1]

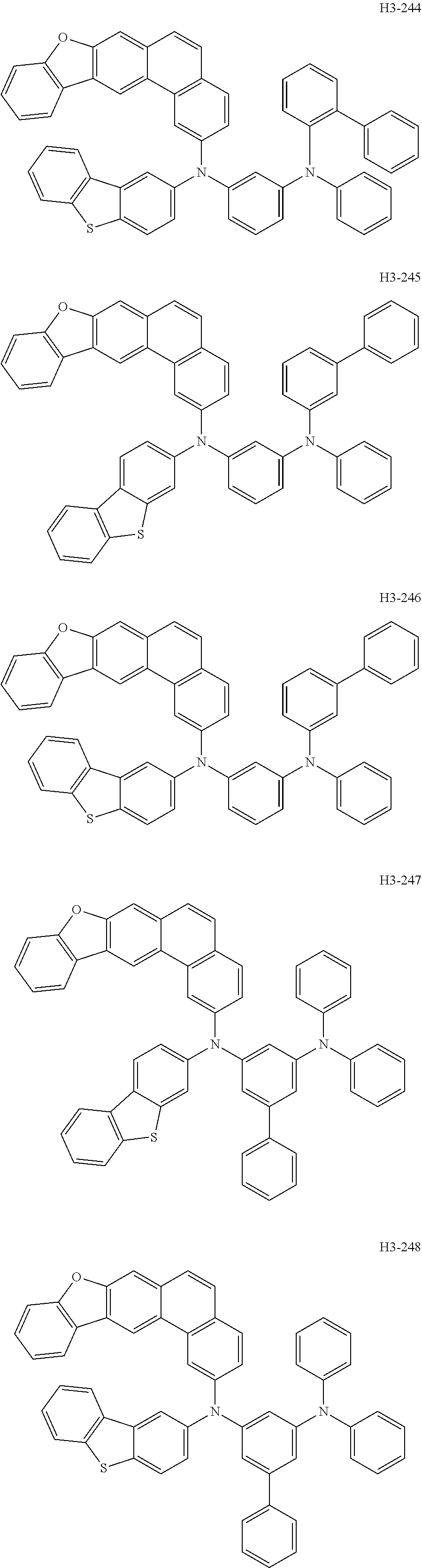 [SMILES: c1ccc(-c2ccccc2N(c2ccccc2)c2cccc(N(c3ccc4ccc5cc6oc7ccccc7c6cc5c4c3)c3ccc4sc5ccccc5c4c3)c2)cc1 ; c1ccc(-c2cccc(N(c3ccccc3)c3cccc(N(c4ccc5c(c4)sc4ccccc45)c4ccc5ccc6cc7oc8ccccc8c7cc6c5c4)c3)c2)cc1 ; c1ccc(-c2cccc(N(c3ccccc3)c3cccc(N(c4ccc5ccc6cc7oc8ccccc8c7cc6c5c4)c4ccc5sc6ccccc6c5c4)c3)c2)cc1 ; c1ccc(-c2cc(N(c3ccccc3)c3ccccc3)cc(N(c3ccc4c(c3)sc3ccccc34)c3ccc4ccc5cc6oc7ccccc7c6cc5c4c3)c2)cc1 ; c1ccc(-c2cc(N(c3ccccc3)c3ccccc3)cc(N(c3ccc4ccc5cc6oc7ccccc7c6cc5c4c3)c3ccc4sc5ccccc5c4c3)c2)cc1]

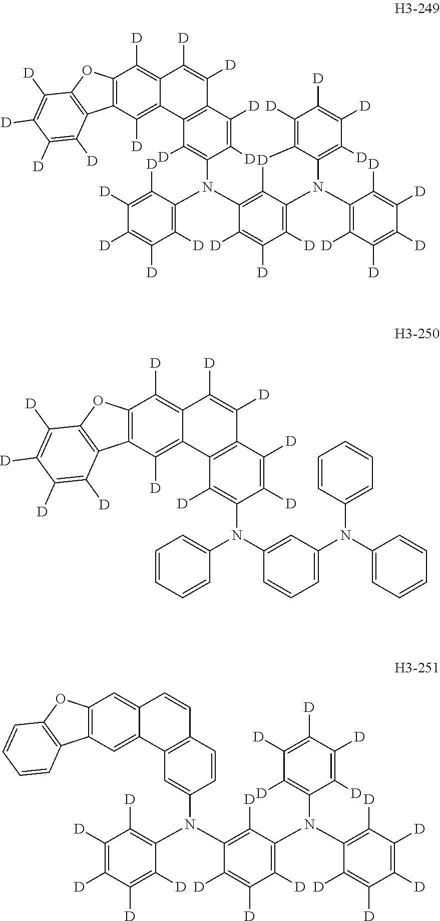

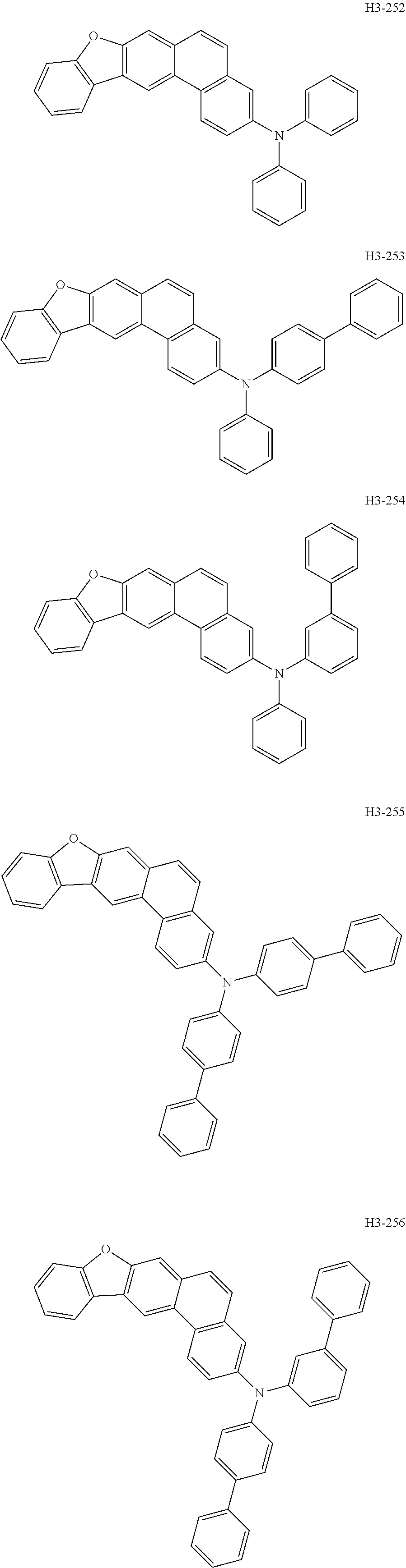 [SMILES: c1ccc(N(c2ccccc2)c2ccc3c(ccc4cc5oc6ccccc6c5cc43)c2)cc1 ; c1ccc(-c2ccc(N(c3ccccc3)c3ccc4c(ccc5cc6oc7ccccc7c6cc54)c3)cc2)cc1 ; c1ccc(-c2cccc(N(c3ccccc3)c3ccc4c(ccc5cc6oc7ccccc7c6cc54)c3)c2)cc1 ; c1ccc(-c2ccc(N(c3ccc(-c4ccccc4)cc3)c3ccc4c(ccc5cc6oc7ccccc7c6cc54)c3)cc2)cc1 ; c1ccc(-c2ccc(N(c3cccc(-c4ccccc4)c3)c3ccc4c(ccc5cc6oc7ccccc7c6cc54)c3)cc2)cc1]

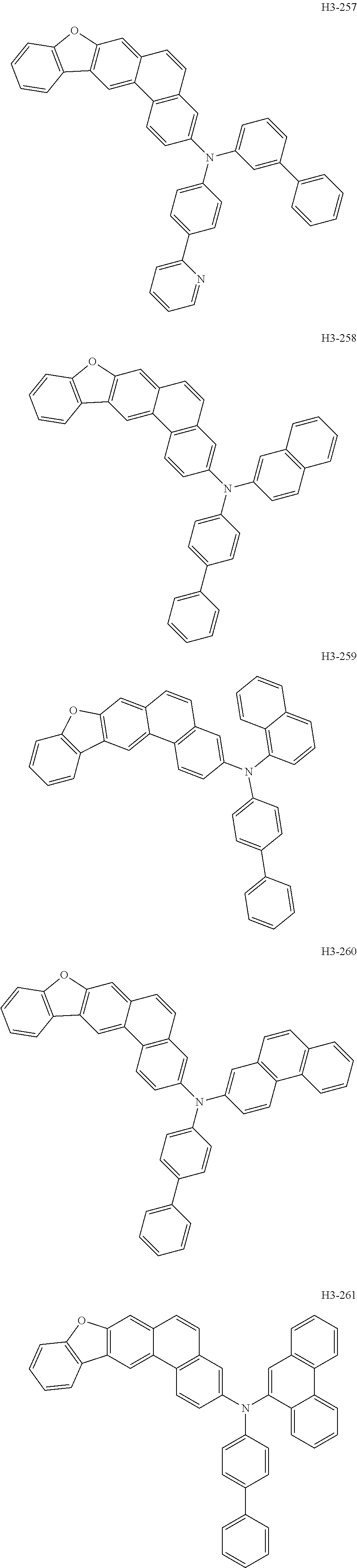 [SMILES: c1ccc(-c2cccc(N(c3ccc(-c4ccccn4)cc3)c3ccc4c(ccc5cc6oc7ccccc7c6cc54)c3)c2)cc1 ; c1ccc(-c2ccc(N(c3ccc4ccccc4c3)c3ccc4c(ccc5cc6oc7ccccc7c6cc54)c3)cc2)cc1 ; c1ccc(-c2ccc(N(c3ccc4c(ccc5cc6oc7ccccc7c6cc54)c3)c3cccc4ccccc34)cc2)cc1 ; c1ccc(-c2ccc(N(c3ccc4c(ccc5ccccc54)c3)c3ccc4c(ccc5cc6oc7ccccc7c6cc54)c3)cc2)cc1 ; c1ccc(-c2ccc(N(c3ccc4c(ccc5cc6oc7ccccc7c6cc54)c3)c3cc4ccccc4c4ccccc34)cc2)cc1]

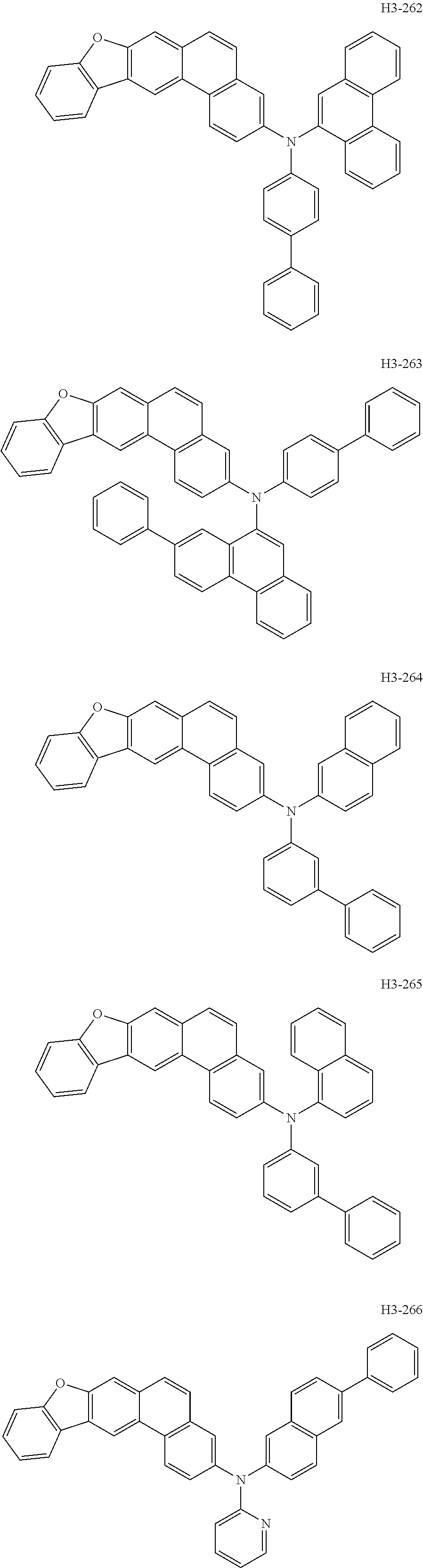 [SMILES: c1ccc(-c2ccc(N(c3ccc4c(ccc5cc6oc7ccccc7c6cc54)c3)c3cc4ccccc4c4ccccc34)cc2)cc1 ; c1ccc(-c2ccc(N(c3ccc4c(ccc5cc6oc7ccccc7c6cc54)c3)c3cc4ccccc4c4ccc(-c5ccccc5)cc34)cc2)cc1 ; c1ccc(-c2cccc(N(c3ccc4ccccc4c3)c3ccc4c(ccc5cc6oc7ccccc7c6cc54)c3)c2)cc1 ; c1ccc(-c2cccc(N(c3ccc4c(ccc5cc6oc7ccccc7c6cc54)c3)c3cccc4ccccc34)c2)cc1 ; c1ccc(-c2ccc3cc(N(c4ccc5c(ccc6cc7oc8ccccc8c7cc65)c4)c4ccccn4)ccc3c2)cc1]

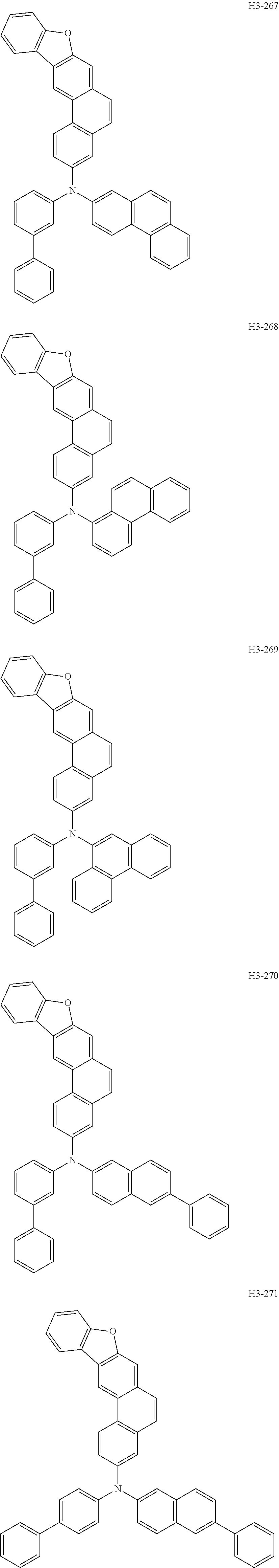

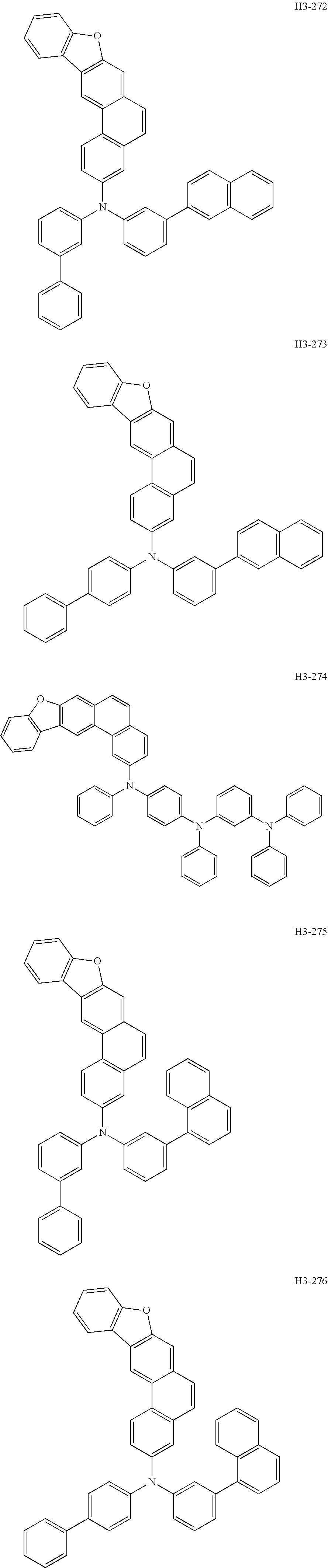 [SMILES: c1ccc(-c2cccc(N(c3cccc(-c4ccc5ccccc5c4)c3)c3ccc4c(ccc5cc6oc7ccccc7c6cc54)c3)c2)cc1 ; c1ccc(-c2ccc(N(c3cccc(-c4ccc5ccccc5c4)c3)c3ccc4c(ccc5cc6oc7ccccc7c6cc54)c3)cc2)cc1 ; c1ccc(N(c2ccccc2)c2cccc(N(c3ccccc3)c3ccc(N(c4ccccc4)c4ccc5ccc6cc7oc8ccccc8c7cc6c5c4)cc3)c2)cc1 ; c1ccc(-c2cccc(N(c3cccc(-c4cccc5ccccc45)c3)c3ccc4c(ccc5cc6oc7ccccc7c6cc54)c3)c2)cc1 ; c1ccc(-c2ccc(N(c3cccc(-c4cccc5ccccc45)c3)c3ccc4c(ccc5cc6oc7ccccc7c6cc54)c3)cc2)cc1]

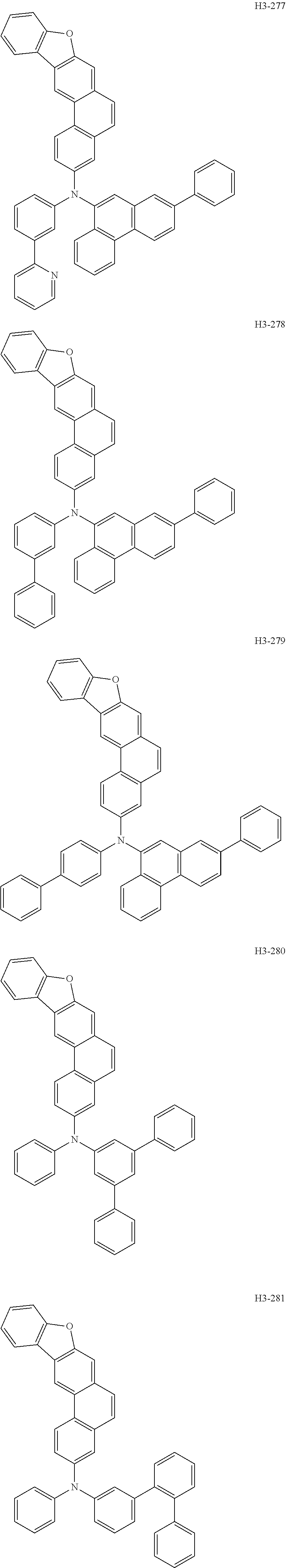

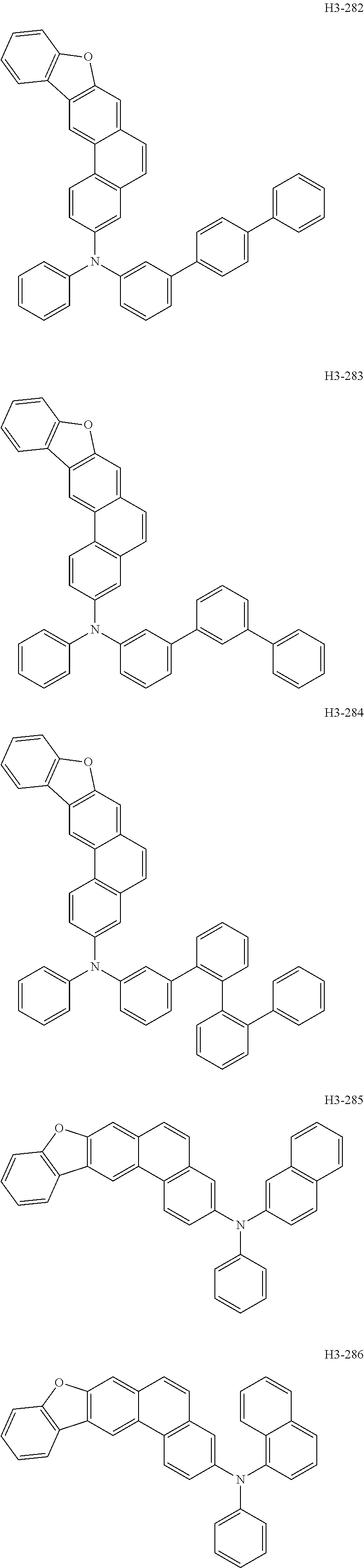 [SMILES: c1ccc(-c2ccc(-c3cccc(N(c4ccccc4)c4ccc5c(ccc6cc7oc8ccccc8c7cc65)c4)c3)cc2)cc1 ; c1ccc(-c2cccc(-c3cccc(N(c4ccccc4)c4ccc5c(ccc6cc7oc8ccccc8c7cc65)c4)c3)c2)cc1 ; c1ccc(-c2ccccc2-c2ccccc2-c2cccc(N(c3ccccc3)c3ccc4c(ccc5cc6oc7ccccc7c6cc54)c3)c2)cc1 ; c1ccc(N(c2ccc3ccccc3c2)c2ccc3c(ccc4cc5oc6ccccc6c5cc43)c2)cc1 ; c1ccc(N(c2ccc3c(ccc4cc5oc6ccccc6c5cc43)c2)c2cccc3ccccc23)cc1]

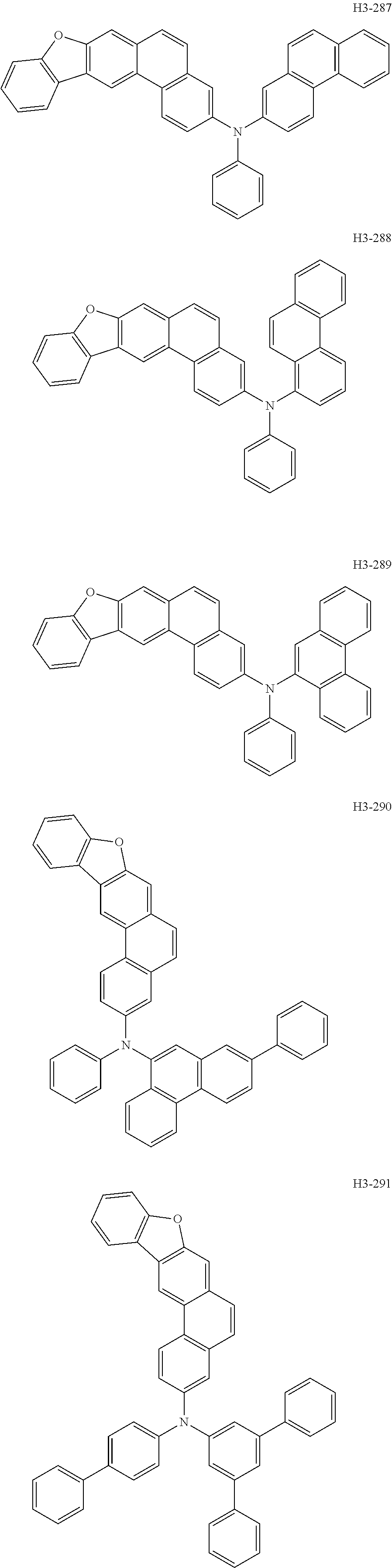 [SMILES: c1ccc(N(c2ccc3c(ccc4ccccc43)c2)c2ccc3c(ccc4cc5oc6ccccc6c5cc43)c2)cc1 ; c1ccc(N(c2ccc3c(ccc4cc5oc6ccccc6c5cc43)c2)c2cccc3c2ccc2ccccc23)cc1 ; c1ccc(N(c2ccc3c(ccc4cc5oc6ccccc6c5cc43)c2)c2cc3ccccc3c3ccccc23)cc1 ; c1ccc(-c2ccc3c(c2)cc(N(c2ccccc2)c2ccc4c(ccc5cc6oc7ccccc7c6cc54)c2)c2ccccc23)cc1 ; c1ccc(-c2ccc(N(c3cc(-c4ccccc4)cc(-c4ccccc4)c3)c3ccc4c(ccc5cc6oc7ccccc7c6cc54)c3)cc2)cc1]

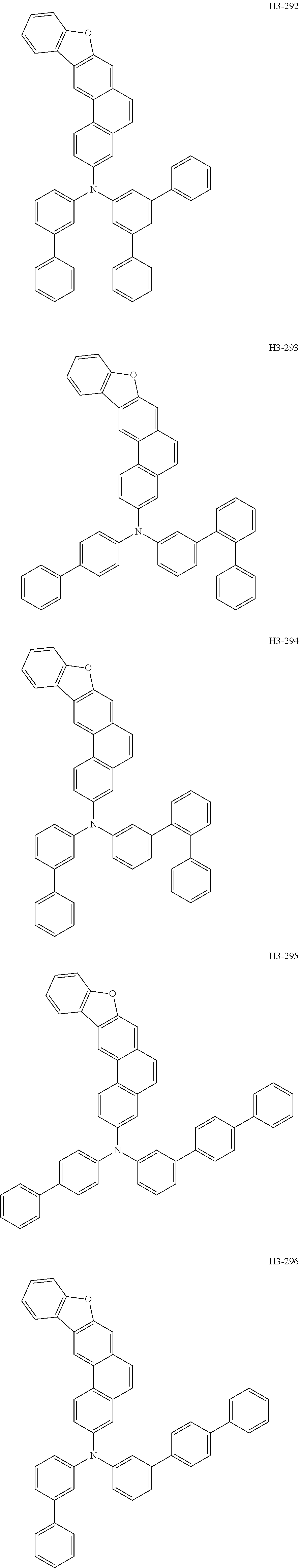 [SMILES: c1ccc(-c2cccc(N(c3cc(-c4ccccc4)cc(-c4ccccc4)c3)c3ccc4c(ccc5cc6oc7ccccc7c6cc54)c3)c2)cc1 ; c1ccc(-c2ccc(N(c3cccc(-c4ccccc4-c4ccccc4)c3)c3ccc4c(ccc5cc6oc7ccccc7c6cc54)c3)cc2)cc1 ; c1ccc(-c2cccc(N(c3cccc(-c4ccccc4-c4ccccc4)c3)c3ccc4c(ccc5cc6oc7ccccc7c6cc54)c3)c2)cc1 ; c1ccc(-c2ccc(-c3cccc(N(c4ccc(-c5ccccc5)cc4)c4ccc5c(ccc6cc7oc8ccccc8c7cc65)c4)c3)cc2)cc1 ; c1ccc(-c2ccc(-c3cccc(N(c4cccc(-c5ccccc5)c4)c4ccc5c(ccc6cc7oc8ccccc8c7cc65)c4)c3)cc2)cc1]

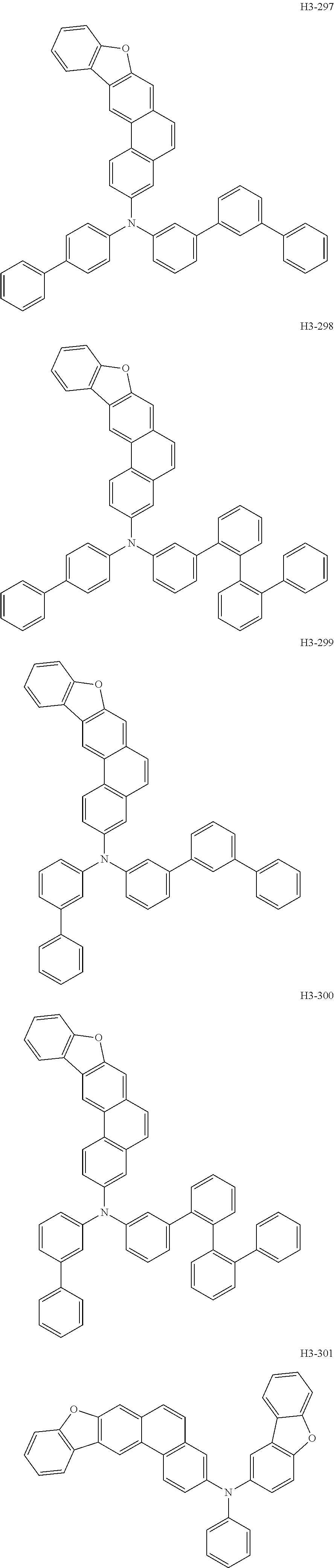

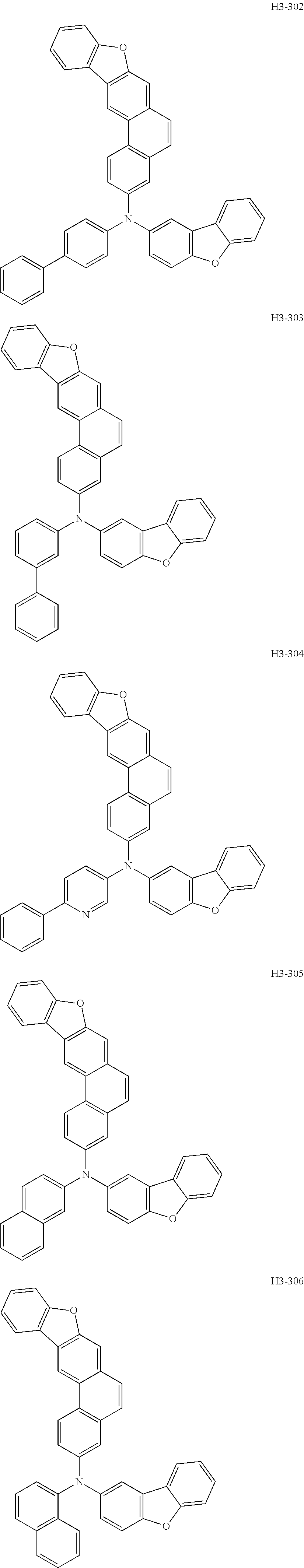

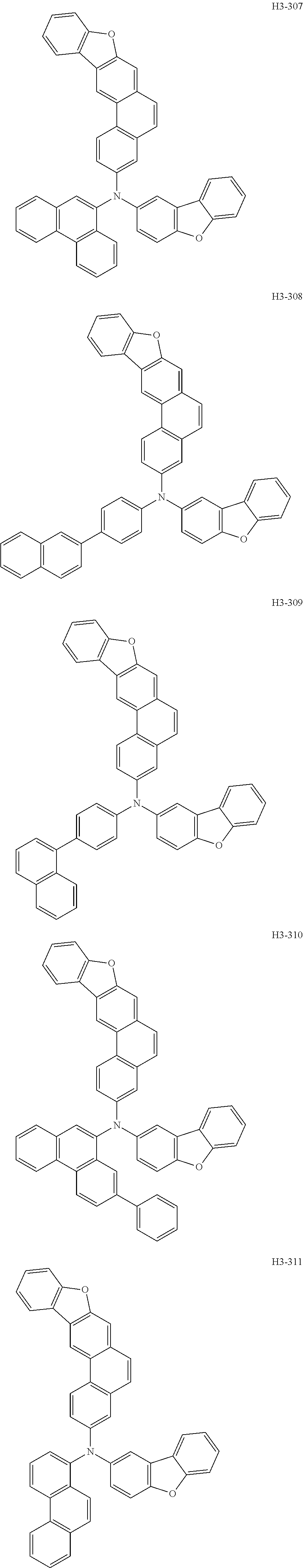

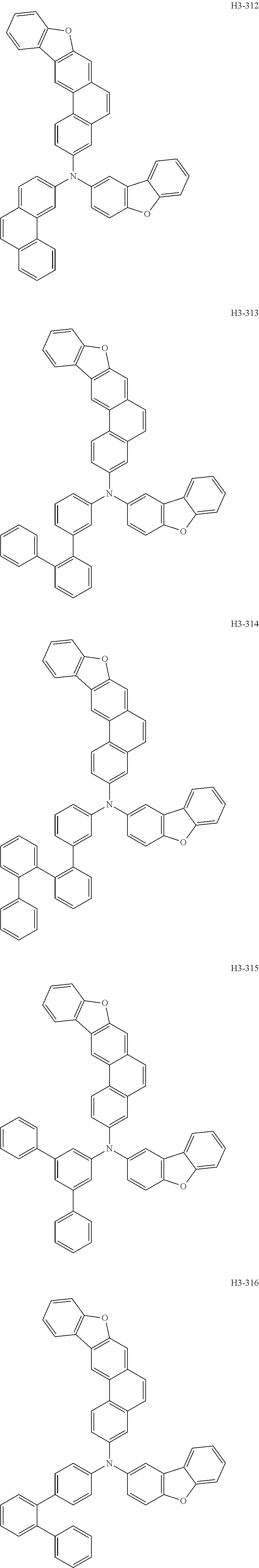 [SMILES: c1ccc2c(c1)ccc1ccc(N(c3ccc4c(ccc5cc6oc7ccccc7c6cc54)c3)c3ccc4oc5ccccc5c4c3)cc12 ; c1ccc(-c2ccccc2-c2cccc(N(c3ccc4c(ccc5cc6oc7ccccc7c6cc54)c3)c3ccc4oc5ccccc5c4c3)c2)cc1 ; c1ccc(-c2ccccc2-c2ccccc2-c2cccc(N(c3ccc4c(ccc5cc6oc7ccccc7c6cc54)c3)c3ccc4oc5ccccc5c4c3)c2)cc1 ; c1ccc(-c2cc(-c3ccccc3)cc(N(c3ccc4c(ccc5cc6oc7ccccc7c6cc54)c3)c3ccc4oc5ccccc5c4c3)c2)cc1 ; c1ccc(-c2ccccc2-c2ccc(N(c3ccc4c(ccc5cc6oc7ccccc7c6cc54)c3)c3ccc4oc5ccccc5c4c3)cc2)cc1]

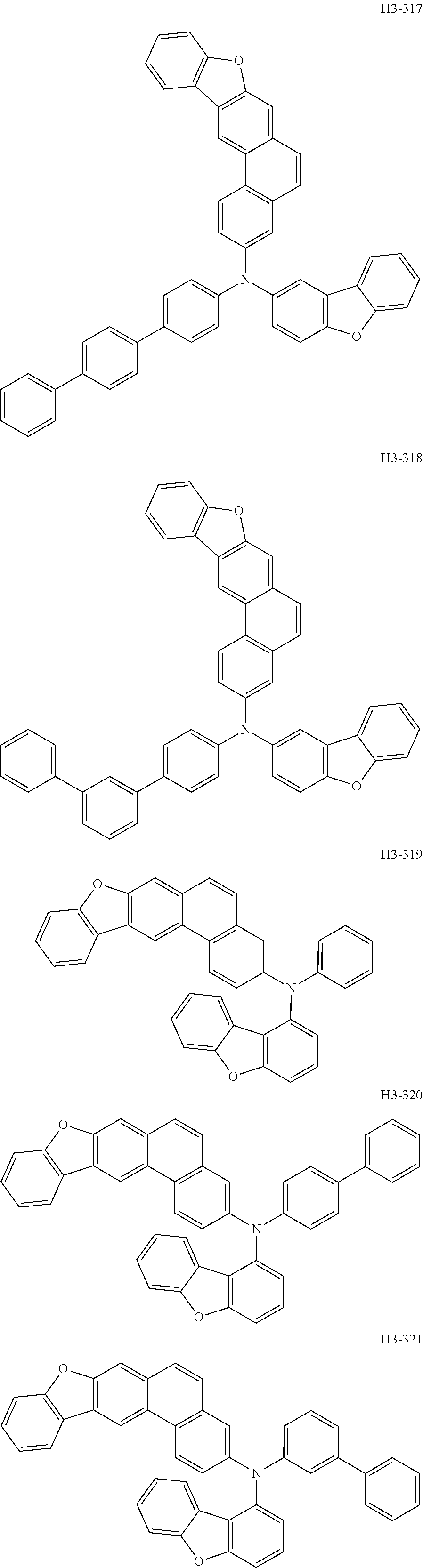 [SMILES: c1ccc(-c2ccc(-c3ccc(N(c4ccc5c(ccc6cc7oc8ccccc8c7cc65)c4)c4ccc5oc6ccccc6c5c4)cc3)cc2)cc1 ; c1ccc(-c2cccc(-c3ccc(N(c4ccc5c(ccc6cc7oc8ccccc8c7cc65)c4)c4ccc5oc6ccccc6c5c4)cc3)c2)cc1 ; c1ccc(N(c2ccc3c(ccc4cc5oc6ccccc6c5cc43)c2)c2cccc3oc4ccccc4c23)cc1 ; c1ccc(-c2ccc(N(c3ccc4c(ccc5cc6oc7ccccc7c6cc54)c3)c3cccc4oc5ccccc5c34)cc2)cc1 ; c1ccc(-c2cccc(N(c3ccc4c(ccc5cc6oc7ccccc7c6cc54)c3)c3cccc4oc5ccccc5c34)c2)cc1]

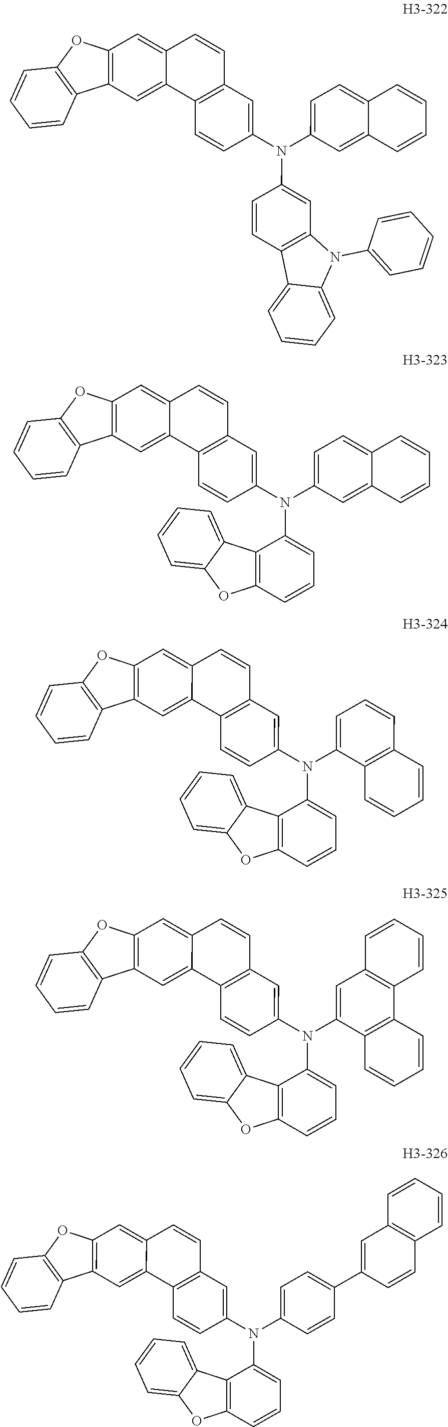 [SMILES: c1ccc(-n2c3ccccc3c3ccc(N(c4ccc5ccccc5c4)c4ccc5c(ccc6cc7oc8ccccc8c7cc65)c4)cc32)cc1 ; c1ccc2cc(N(c3ccc4c(ccc5cc6oc7ccccc7c6cc54)c3)c3cccc4oc5ccccc5c34)ccc2c1 ; c1ccc2c(N(c3ccc4c(ccc5cc6oc7ccccc7c6cc54)c3)c3cccc4oc5ccccc5c34)cccc2c1 ; c1ccc2c(c1)cc(N(c1ccc3c(ccc4cc5oc6ccccc6c5cc43)c1)c1cccc3oc4ccccc4c13)c1ccccc12 ; c1ccc2cc(-c3ccc(N(c4ccc5c(ccc6cc7oc8ccccc8c7cc65)c4)c4cccc5oc6ccccc6c45)cc3)ccc2c1]

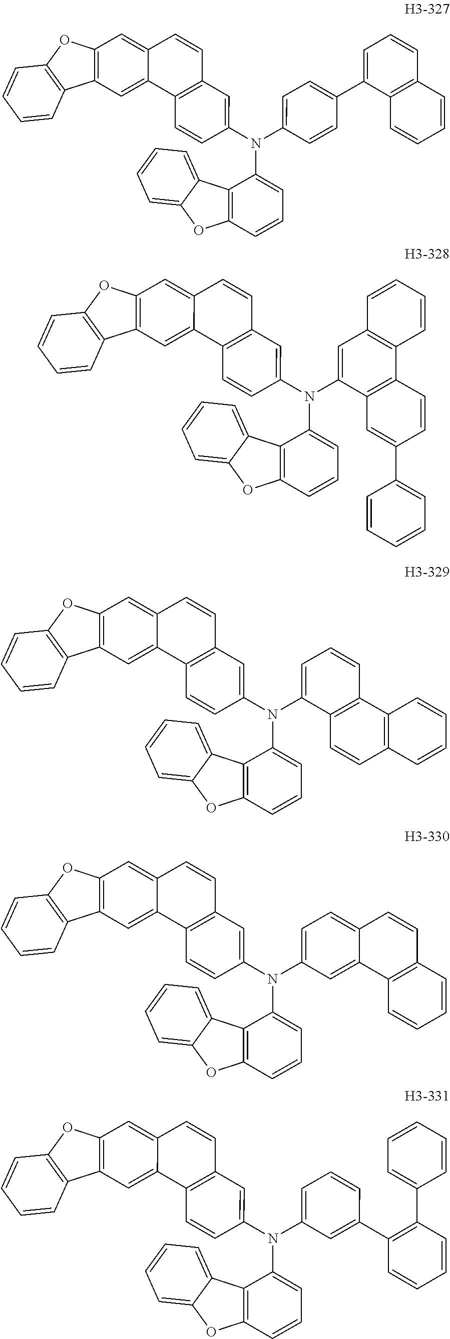 [SMILES: c1ccc2c(-c3ccc(N(c4ccc5c(ccc6cc7oc8ccccc8c7cc65)c4)c4cccc5oc6ccccc6c45)cc3)cccc2c1 ; c1ccc(-c2ccc3c(c2)c(N(c2ccc4c(ccc5cc6oc7ccccc7c6cc54)c2)c2cccc4oc5ccccc5c24)cc2ccccc23)cc1 ; c1ccc2c(c1)ccc1c(N(c3ccc4c(ccc5cc6oc7ccccc7c6cc54)c3)c3cccc4oc5ccccc5c34)cccc12 ; c1ccc2c(c1)ccc1ccc(N(c3ccc4c(ccc5cc6oc7ccccc7c6cc54)c3)c3cccc4oc5ccccc5c34)cc12 ; c1ccc(-c2ccccc2-c2cccc(N(c3ccc4c(ccc5cc6oc7ccccc7c6cc54)c3)c3cccc4oc5ccccc5c34)c2)cc1]

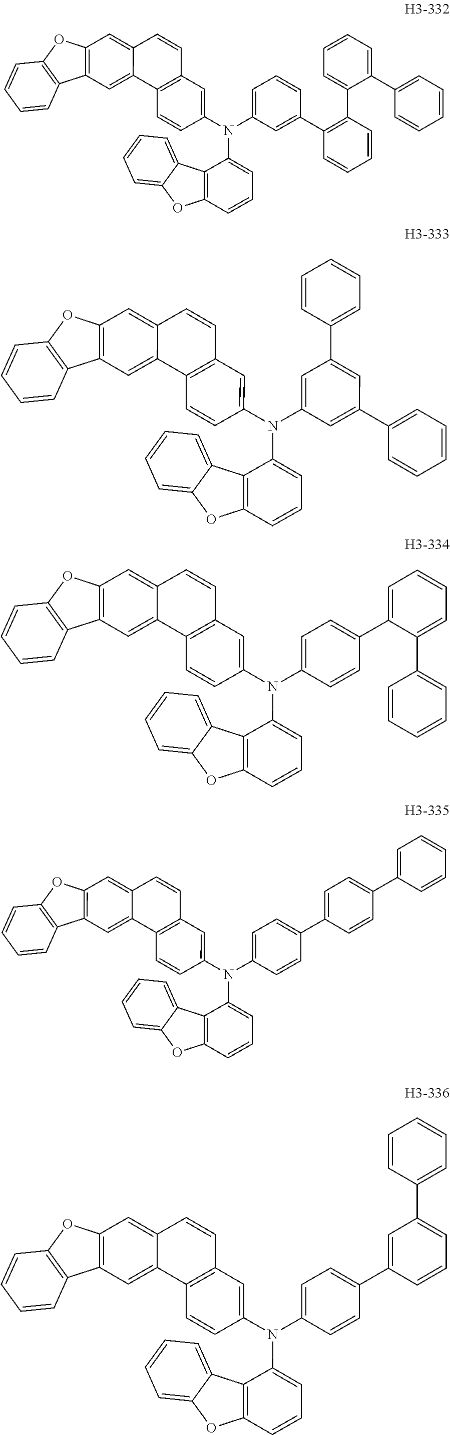 [SMILES: c1ccc(-c2ccccc2-c2ccccc2-c2cccc(N(c3ccc4c(ccc5cc6oc7ccccc7c6cc54)c3)c3cccc4oc5ccccc5c34)c2)cc1 ; c1ccc(-c2cc(-c3ccccc3)cc(N(c3ccc4c(ccc5cc6oc7ccccc7c6cc54)c3)c3cccc4oc5ccccc5c34)c2)cc1 ; c1ccc(-c2ccccc2-c2ccc(N(c3ccc4c(ccc5cc6oc7ccccc7c6cc54)c3)c3cccc4oc5ccccc5c34)cc2)cc1 ; c1ccc(-c2ccc(-c3ccc(N(c4ccc5c(ccc6cc7oc8ccccc8c7cc65)c4)c4cccc5oc6ccccc6c45)cc3)cc2)cc1 ; c1ccc(-c2cccc(-c3ccc(N(c4ccc5c(ccc6cc7oc8ccccc8c7cc65)c4)c4cccc5oc6ccccc6c45)cc3)c2)cc1]

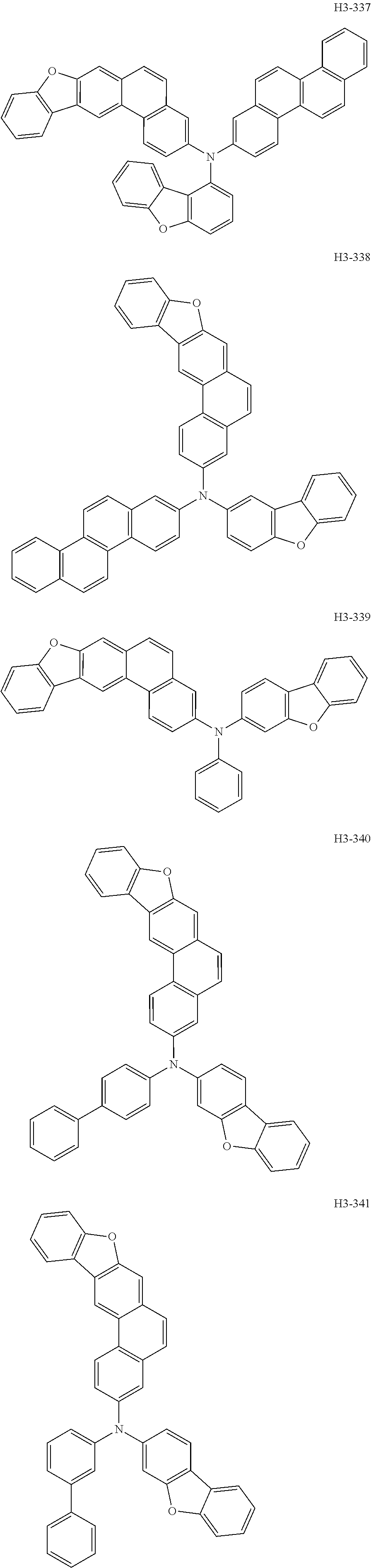 [SMILES: c1ccc2c(c1)ccc1c3ccc(N(c4ccc5c(ccc6cc7oc8ccccc8c7cc65)c4)c4cccc5oc6ccccc6c45)cc3ccc21 ; c1ccc2c(c1)ccc1c3ccc(N(c4ccc5c(ccc6cc7oc8ccccc8c7cc65)c4)c4ccc5oc6ccccc6c5c4)cc3ccc21 ; c1ccc(N(c2ccc3c(ccc4cc5oc6ccccc6c5cc43)c2)c2ccc3c(c2)oc2ccccc23)cc1 ; c1ccc(-c2ccc(N(c3ccc4c(ccc5cc6oc7ccccc7c6cc54)c3)c3ccc4c(c3)oc3ccccc34)cc2)cc1 ; c1ccc(-c2cccc(N(c3ccc4c(ccc5cc6oc7ccccc7c6cc54)c3)c3ccc4c(c3)oc3ccccc34)c2)cc1]

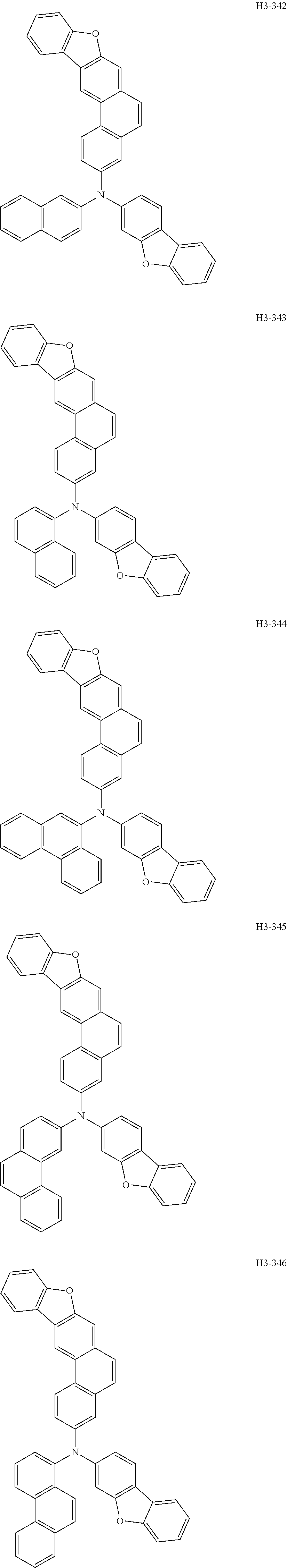 [SMILES: c1ccc2cc(N(c3ccc4c(ccc5cc6oc7ccccc7c6cc54)c3)c3ccc4c(c3)oc3ccccc34)ccc2c1 ; c1ccc2c(N(c3ccc4c(ccc5cc6oc7ccccc7c6cc54)c3)c3ccc4c(c3)oc3ccccc34)cccc2c1 ; c1ccc2c(c1)cc(N(c1ccc3c(ccc4cc5oc6ccccc6c5cc43)c1)c1ccc3c(c1)oc1ccccc13)c1ccccc12 ; c1ccc2c(c1)ccc1ccc(N(c3ccc4c(ccc5cc6oc7ccccc7c6cc54)c3)c3ccc4c(c3)oc3ccccc34)cc12 ; c1ccc2c(c1)ccc1c(N(c3ccc4c(ccc5cc6oc7ccccc7c6cc54)c3)c3ccc4c(c3)oc3ccccc34)cccc12]

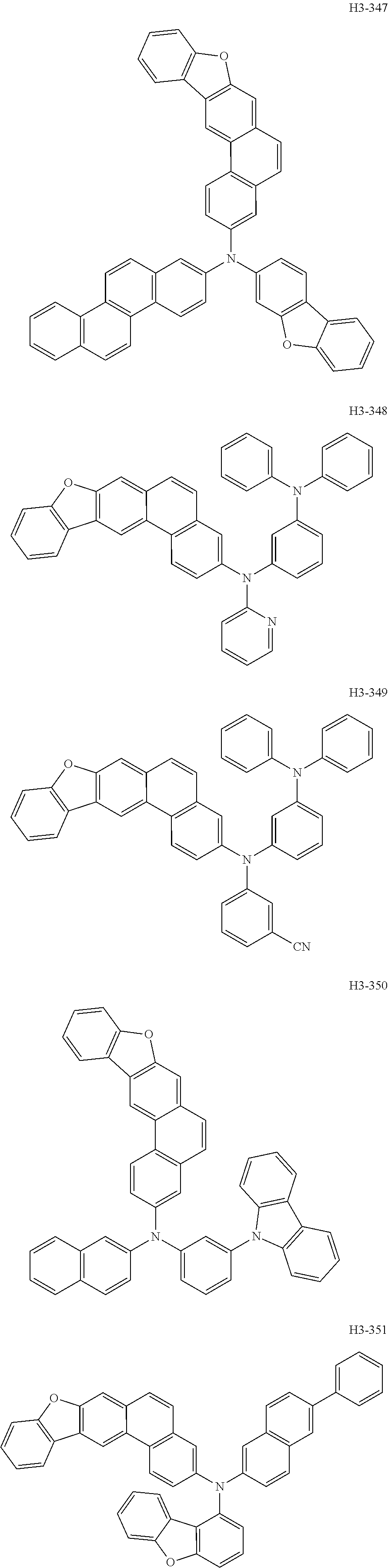 [SMILES: c1ccc2c(c1)ccc1c3ccc(N(c4ccc5c(ccc6cc7oc8ccccc8c7cc65)c4)c4ccc5c(c4)oc4ccccc45)cc3ccc21 ; c1ccc(N(c2ccccc2)c2cccc(N(c3ccc4c(ccc5cc6oc7ccccc7c6cc54)c3)c3ccccn3)c2)cc1 ; N#Cc1cccc(N(c2cccc(N(c3ccccc3)c3ccccc3)c2)c2ccc3c(ccc4cc5oc6ccccc6c5cc43)c2)c1 ; c1cc(N(c2ccc3ccccc3c2)c2ccc3c(ccc4cc5oc6ccccc6c5cc43)c2)cc(-n2c3ccccc3c3ccccc32)c1 ; c1ccc(-c2ccc3cc(N(c4ccc5c(ccc6cc7oc8ccccc8c7cc65)c4)c4cccc5oc6ccccc6c45)ccc3c2)cc1]

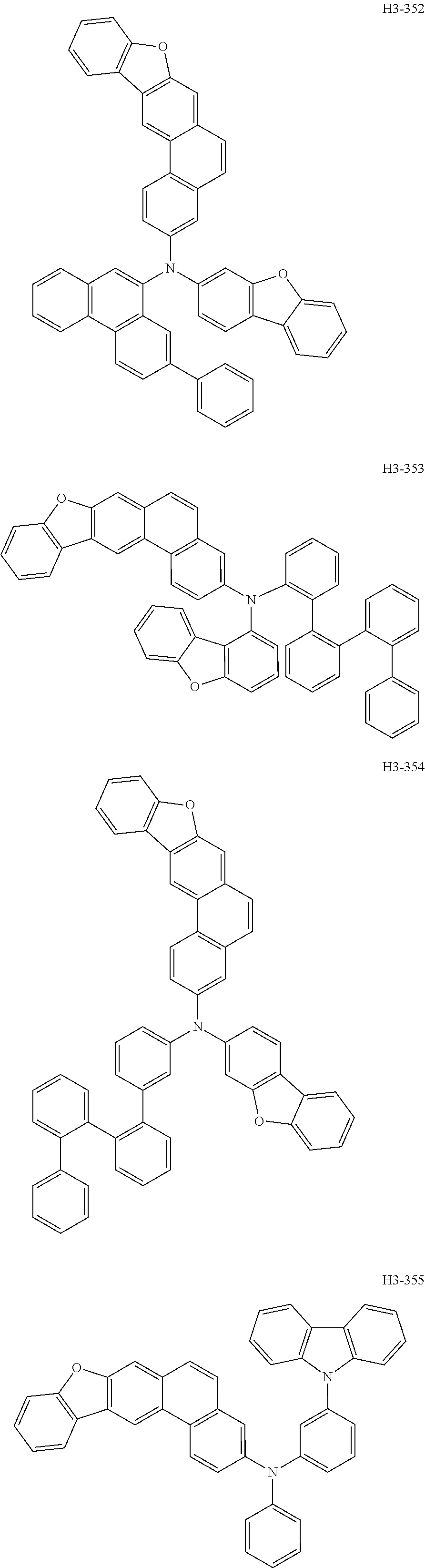 [SMILES: c1ccc(-c2ccc3c(c2)c(N(c2ccc4c(ccc5cc6oc7ccccc7c6cc54)c2)c2ccc4c(c2)oc2ccccc24)cc2ccccc23)cc1 ; c1ccc(-c2ccccc2-c2ccccc2-c2ccccc2N(c2ccc3c(ccc4cc5oc6ccccc6c5cc43)c2)c2cccc3oc4ccccc4c23)cc1 ; c1ccc(-c2ccccc2-c2ccccc2-c2cccc(N(c3ccc4c(ccc5cc6oc7ccccc7c6cc54)c3)c3ccc4c(c3)oc3ccccc34)c2)cc1 ; c1ccc(N(c2cccc(-n3c4ccccc4c4ccccc43)c2)c2ccc3c(ccc4cc5oc6ccccc6c5cc43)c2)cc1]

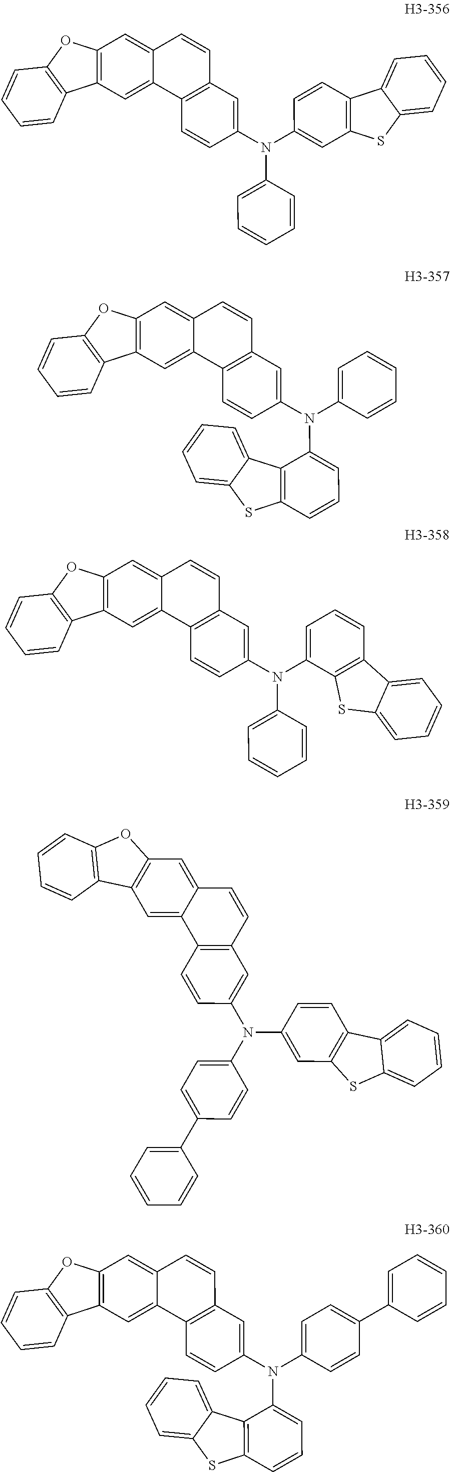 [SMILES: c1ccc(N(c2ccc3c(ccc4cc5oc6ccccc6c5cc43)c2)c2ccc3c(c2)sc2ccccc23)cc1 ; c1ccc(N(c2ccc3c(ccc4cc5oc6ccccc6c5cc43)c2)c2cccc3sc4ccccc4c23)cc1 ; c1ccc(N(c2ccc3c(ccc4cc5oc6ccccc6c5cc43)c2)c2cccc3c2sc2ccccc23)cc1 ; c1ccc(-c2ccc(N(c3ccc4c(ccc5cc6oc7ccccc7c6cc54)c3)c3ccc4c(c3)sc3ccccc34)cc2)cc1 ; c1ccc(-c2ccc(N(c3ccc4c(ccc5cc6oc7ccccc7c6cc54)c3)c3cccc4sc5ccccc5c34)cc2)cc1]

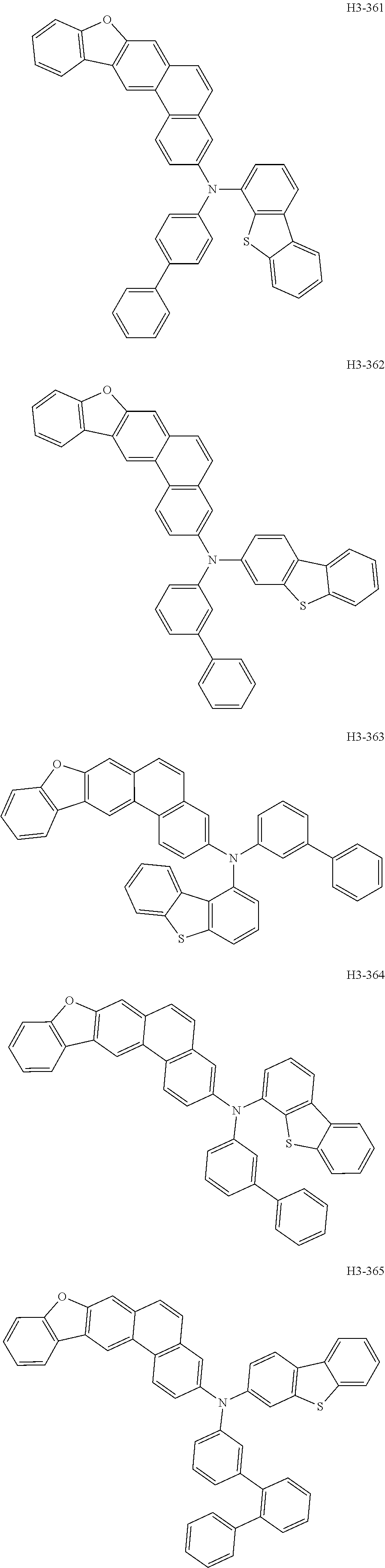 [SMILES: c1ccc(-c2ccc(N(c3ccc4c(ccc5cc6oc7ccccc7c6cc54)c3)c3cccc4c3sc3ccccc34)cc2)cc1 ; c1ccc(-c2cccc(N(c3ccc4c(ccc5cc6oc7ccccc7c6cc54)c3)c3ccc4c(c3)sc3ccccc34)c2)cc1 ; c1ccc(-c2cccc(N(c3ccc4c(ccc5cc6oc7ccccc7c6cc54)c3)c3cccc4sc5ccccc5c34)c2)cc1 ; c1ccc(-c2cccc(N(c3ccc4c(ccc5cc6oc7ccccc7c6cc54)c3)c3cccc4c3sc3ccccc34)c2)cc1 ; c1ccc(-c2ccccc2-c2cccc(N(c3ccc4c(ccc5cc6oc7ccccc7c6cc54)c3)c3ccc4c(c3)sc3ccccc34)c2)cc1]

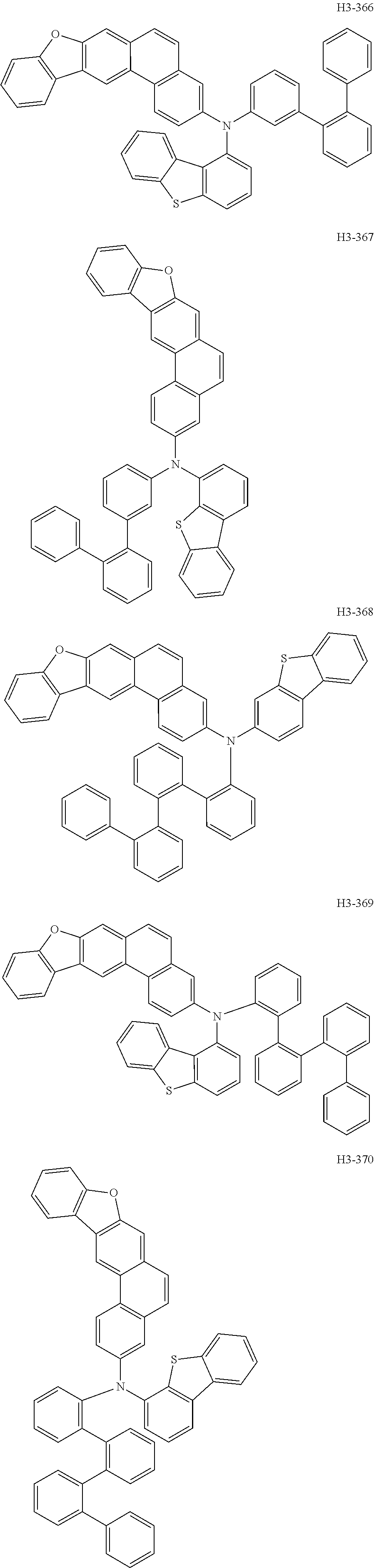 [SMILES: c1ccc(-c2ccccc2-c2cccc(N(c3ccc4c(ccc5cc6oc7ccccc7c6cc54)c3)c3cccc4sc5ccccc5c34)c2)cc1 ; c1ccc(-c2ccccc2-c2cccc(N(c3ccc4c(ccc5cc6oc7ccccc7c6cc54)c3)c3cccc4c3sc3ccccc34)c2)cc1 ; c1ccc(-c2ccccc2-c2ccccc2-c2ccccc2N(c2ccc3c(ccc4cc5oc6ccccc6c5cc43)c2)c2ccc3c(c2)sc2ccccc23)cc1 ; c1ccc(-c2ccccc2-c2ccccc2-c2ccccc2N(c2ccc3c(ccc4cc5oc6ccccc6c5cc43)c2)c2cccc3sc4ccccc4c23)cc1 ; c1ccc(-c2ccccc2-c2ccccc2-c2ccccc2N(c2ccc3c(ccc4cc5oc6ccccc6c5cc43)c2)c2cccc3c2sc2ccccc23)cc1]

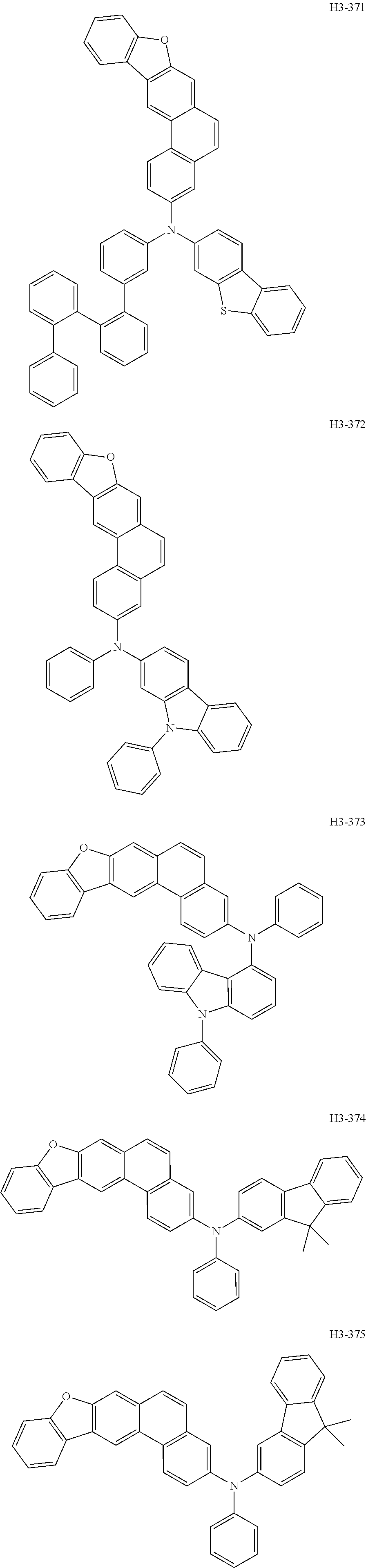

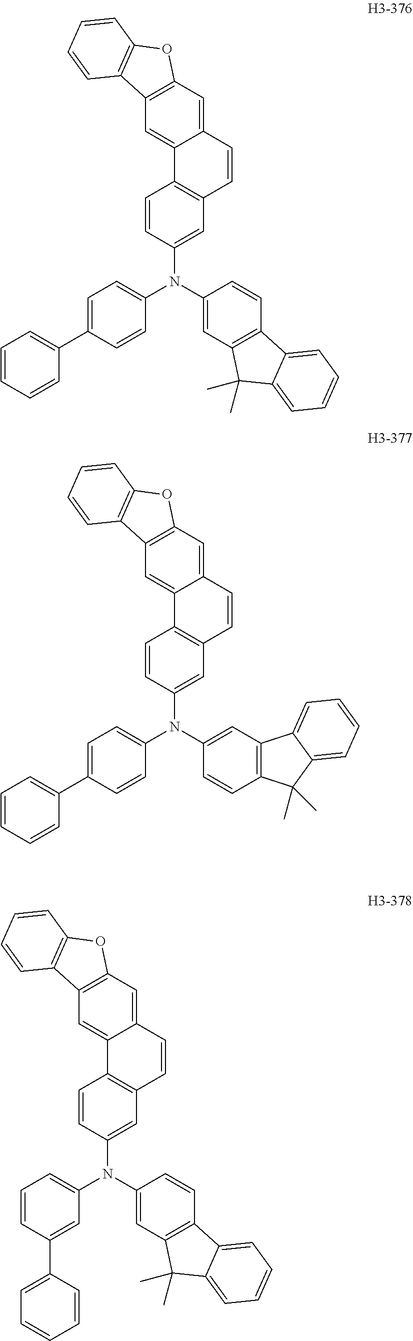 [SMILES: CC1(C)c2ccccc2-c2ccc(N(c3ccc(-c4ccccc4)cc3)c3ccc4c(ccc5cc6oc7ccccc7c6cc54)c3)cc21 ; CC1(C)c2ccccc2-c2cc(N(c3ccc(-c4ccccc4)cc3)c3ccc4c(ccc5cc6oc7ccccc7c6cc54)c3)ccc21 ; CC1(C)c2ccccc2-c2ccc(N(c3cccc(-c4ccccc4)c3)c3ccc4c(ccc5cc6oc7ccccc7c6cc54)c3)cc21]

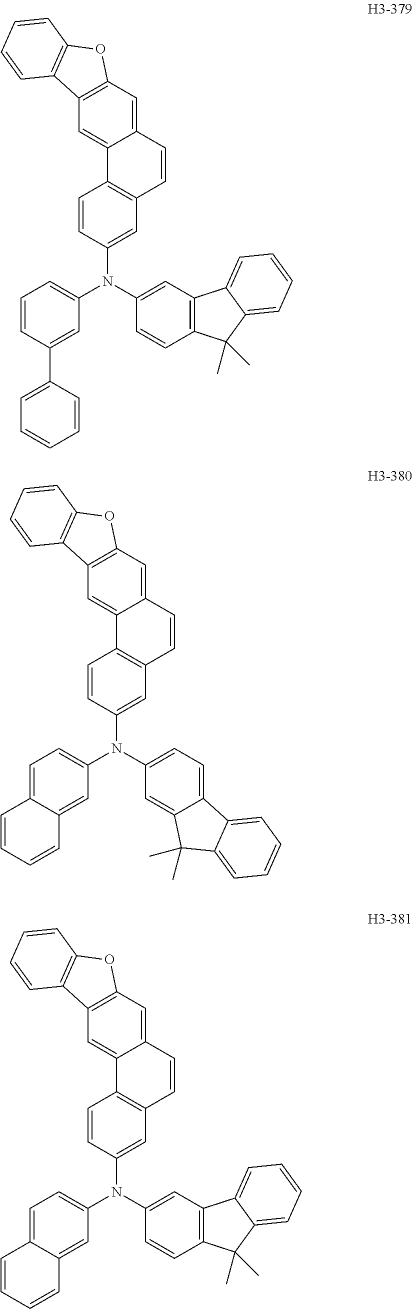

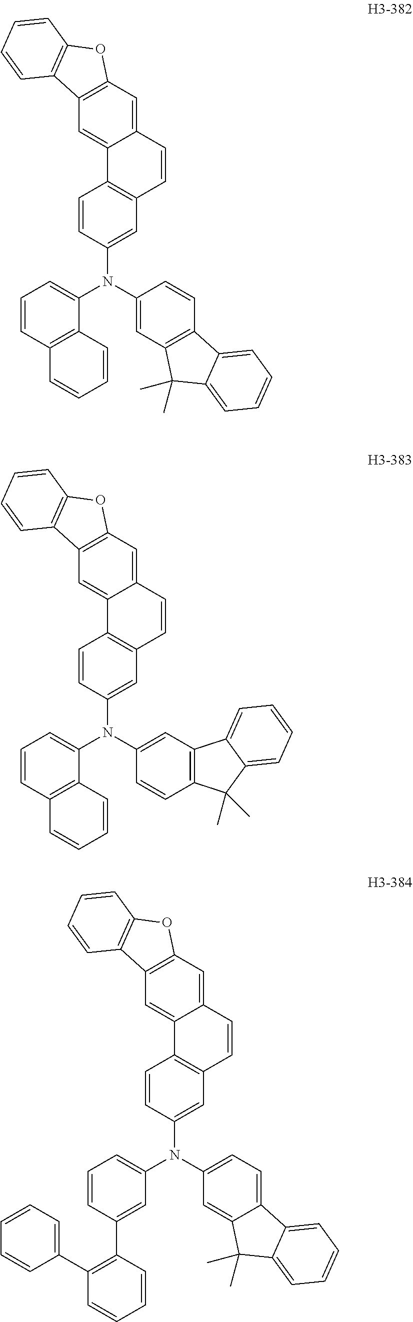

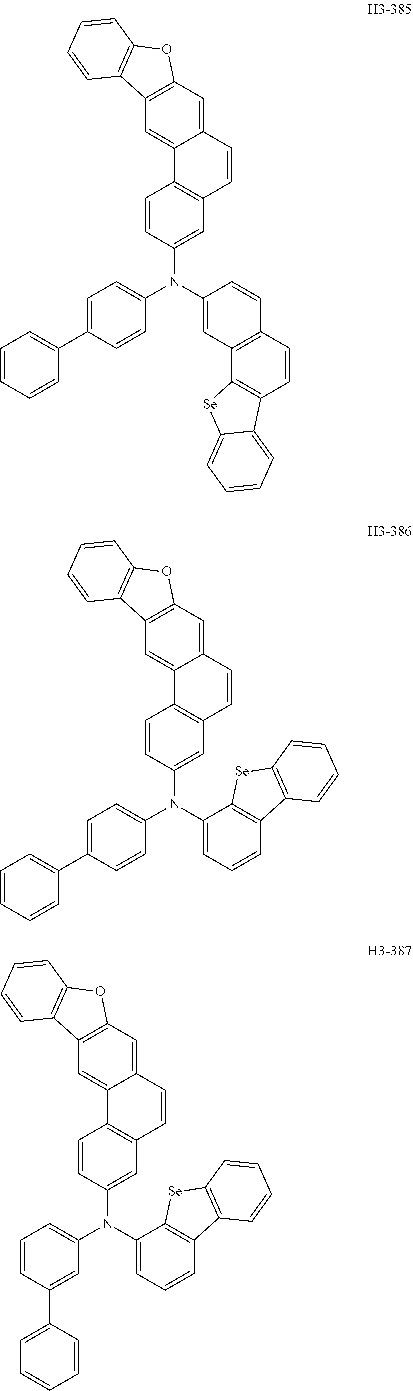

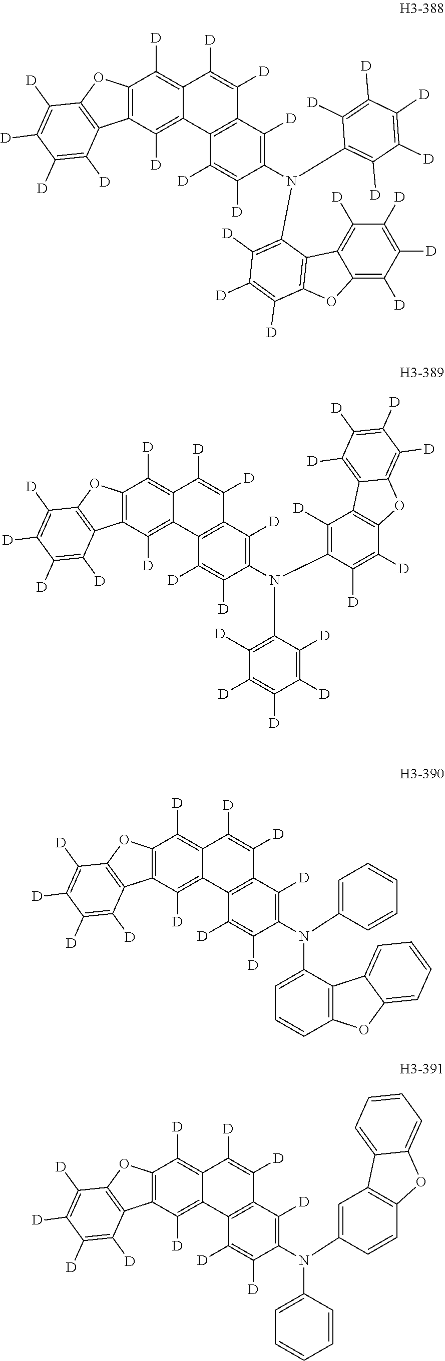

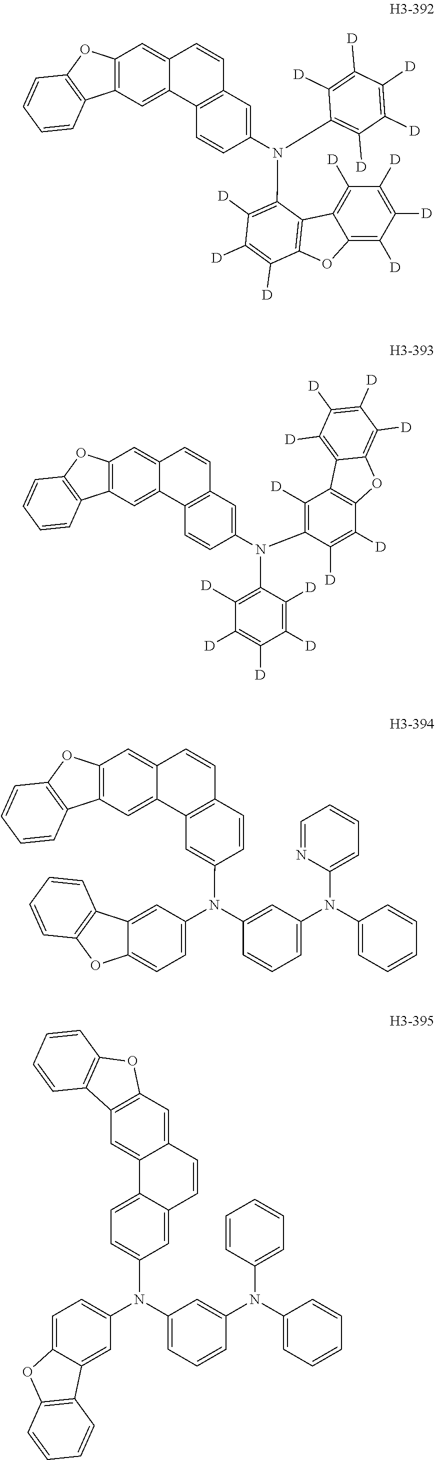 [SMILES: [2H]c1c([2H])c([2H])c(N(c2ccc3c(ccc4cc5oc6ccccc6c5cc43)c2)c2c([2H])c([2H])c([2H])c3oc4c([2H])c([2H])c([2H])c([2H])c4c23)c([2H])c1[2H] ; [2H]c1c([2H])c([2H])c(N(c2ccc3c(ccc4cc5oc6ccccc6c5cc43)c2)c2c([2H])c([2H])c3oc4c([2H])c([2H])c([2H])c([2H])c4c3c2[2H])c([2H])c1[2H] ; c1ccc(N(c2cccc(N(c3ccc4ccc5cc6oc7ccccc7c6cc5c4c3)c3ccc4oc5ccccc5c4c3)c2)c2ccccn2)cc1 ; c1ccc(N(c2ccccc2)c2cccc(N(c3ccc4c(ccc5cc6oc7ccccc7c6cc54)c3)c3ccc4oc5ccccc5c4c3)c2)cc1]

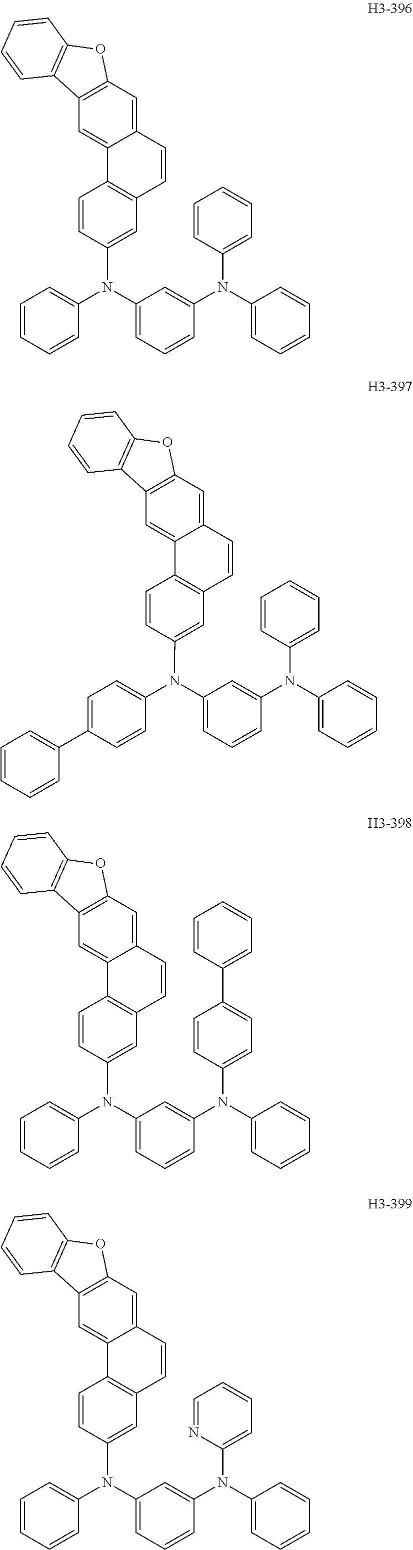

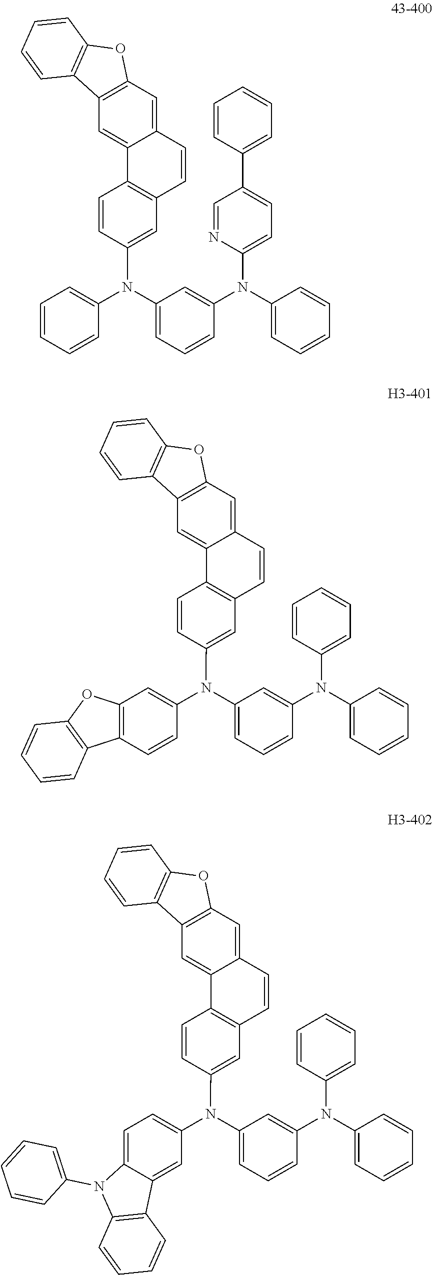 [SMILES: c1ccc(-c2ccc(N(c3ccccc3)c3cccc(N(c4ccccc4)c4ccc5c(ccc6cc7oc8ccccc8c7cc65)c4)c3)nc2)cc1 ; c1ccc(N(c2ccccc2)c2cccc(N(c3ccc4c(ccc5cc6oc7ccccc7c6cc54)c3)c3ccc4c(c3)oc3ccccc34)c2)cc1 ; c1ccc(N(c2ccccc2)c2cccc(N(c3ccc4c(ccc5cc6oc7ccccc7c6cc54)c3)c3ccc4c(c3)c3ccccc3n4-c3ccccc3)c2)cc1]

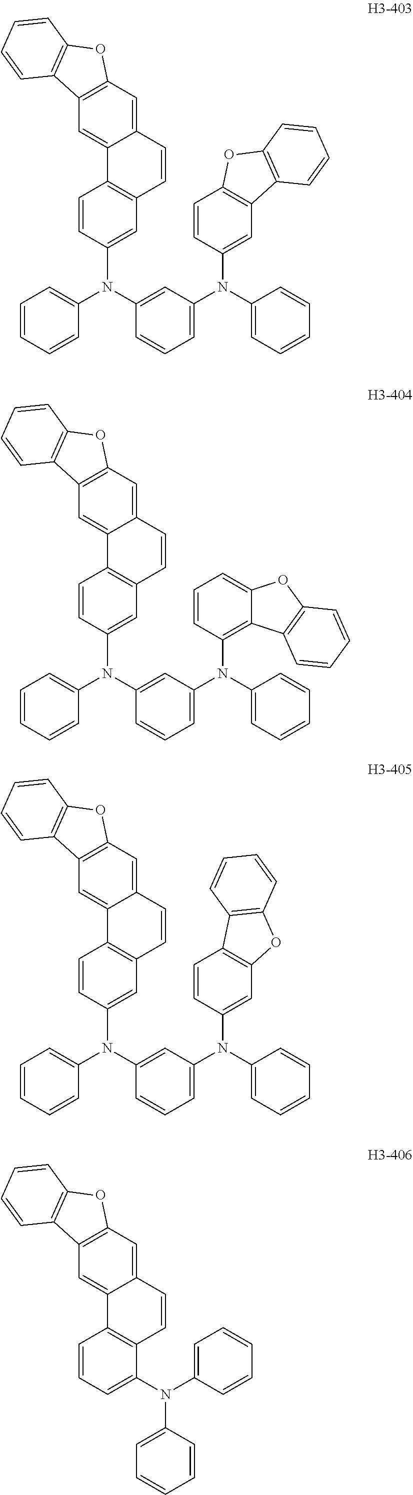

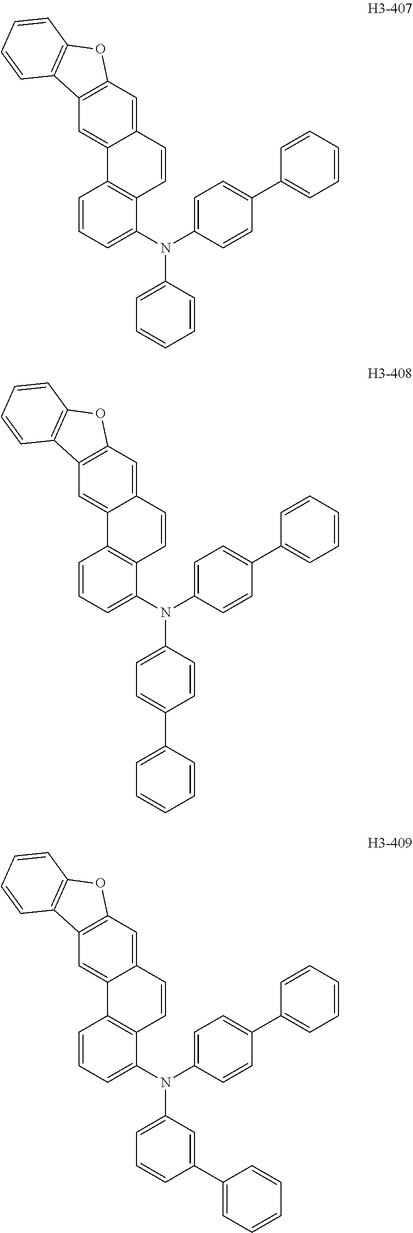 [SMILES: c1ccc(-c2ccc(N(c3ccccc3)c3cccc4c3ccc3cc5oc6ccccc6c5cc34)cc2)cc1 ; c1ccc(-c2ccc(N(c3ccc(-c4ccccc4)cc3)c3cccc4c3ccc3cc5oc6ccccc6c5cc34)cc2)cc1 ; c1ccc(-c2ccc(N(c3cccc(-c4ccccc4)c3)c3cccc4c3ccc3cc5oc6ccccc6c5cc34)cc2)cc1]

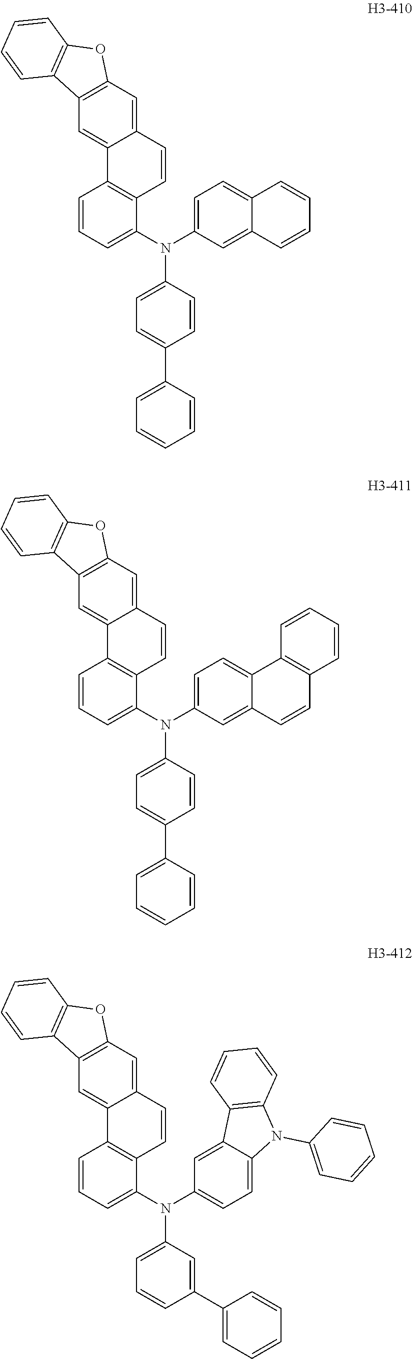 [SMILES: c1ccc(-c2ccc(N(c3ccc4ccccc4c3)c3cccc4c3ccc3cc5oc6ccccc6c5cc34)cc2)cc1 ; c1ccc(-c2ccc(N(c3ccc4c(ccc5ccccc54)c3)c3cccc4c3ccc3cc5oc6ccccc6c5cc34)cc2)cc1 ; c1ccc(-c2cccc(N(c3ccc4c(c3)c3ccccc3n4-c3ccccc3)c3cccc4c3ccc3cc5oc6ccccc6c5cc34)c2)cc1]

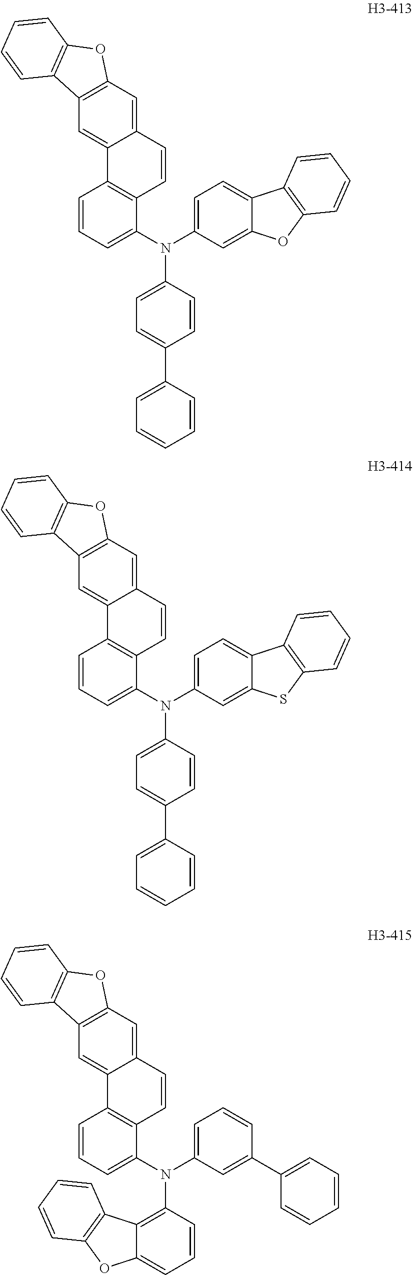 [SMILES: c1ccc(-c2ccc(N(c3ccc4c(c3)oc3ccccc34)c3cccc4c3ccc3cc5oc6ccccc6c5cc34)cc2)cc1 ; c1ccc(-c2ccc(N(c3ccc4c(c3)sc3ccccc34)c3cccc4c3ccc3cc5oc6ccccc6c5cc34)cc2)cc1 ; c1ccc(-c2cccc(N(c3cccc4c3ccc3cc5oc6ccccc6c5cc34)c3cccc4oc5ccccc5c34)c2)cc1]

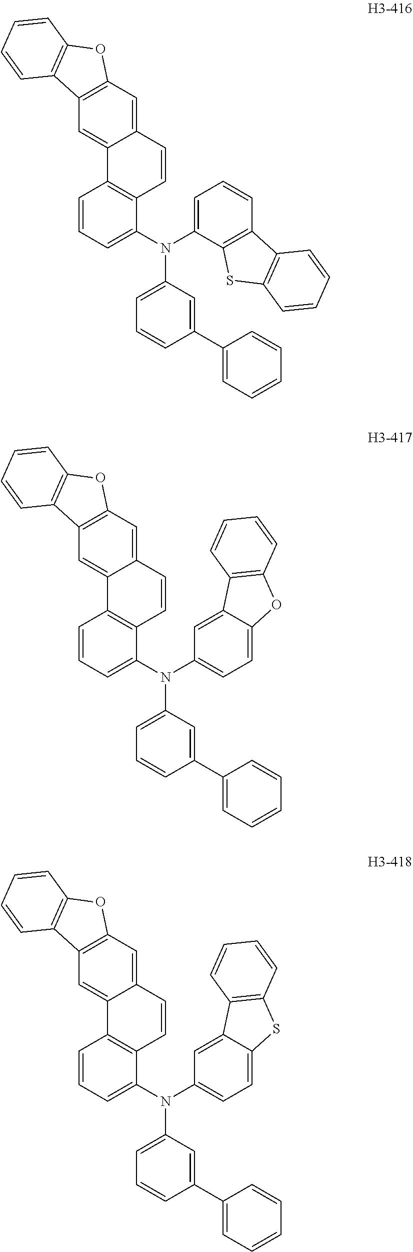 [SMILES: c1ccc(-c2cccc(N(c3cccc4c3ccc3cc5oc6ccccc6c5cc34)c3cccc4c3sc3ccccc34)c2)cc1 ; c1ccc(-c2cccc(N(c3ccc4oc5ccccc5c4c3)c3cccc4c3ccc3cc5oc6ccccc6c5cc34)c2)cc1 ; c1ccc(-c2cccc(N(c3ccc4sc5ccccc5c4c3)c3cccc4c3ccc3cc5oc6ccccc6c5cc34)c2)cc1]

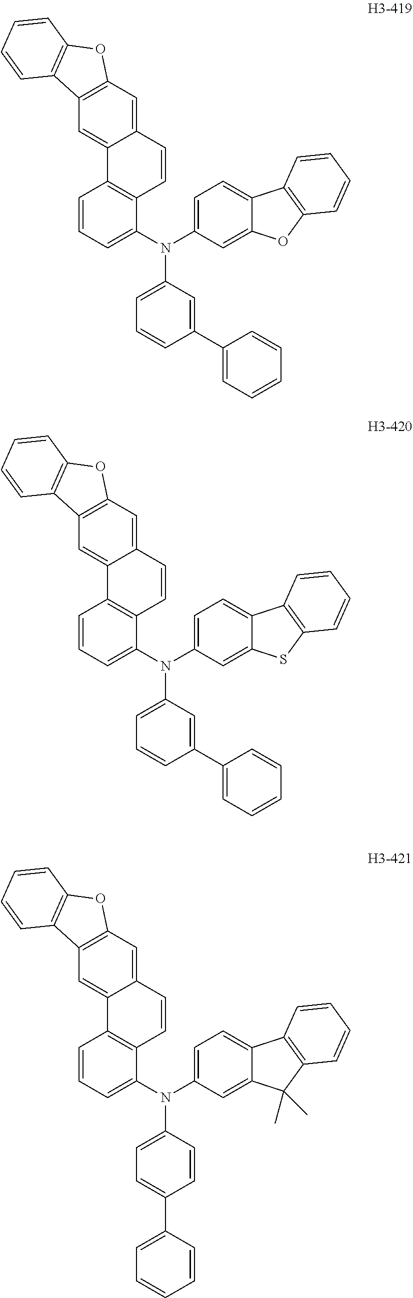 [SMILES: c1ccc(-c2cccc(N(c3ccc4c(c3)oc3ccccc34)c3cccc4c3ccc3cc5oc6ccccc6c5cc34)c2)cc1 ; c1ccc(-c2cccc(N(c3ccc4c(c3)sc3ccccc34)c3cccc4c3ccc3cc5oc6ccccc6c5cc34)c2)cc1 ; CC1(C)c2ccccc2-c2ccc(N(c3ccc(-c4ccccc4)cc3)c3cccc4c3ccc3cc5oc6ccccc6c5cc34)cc21]

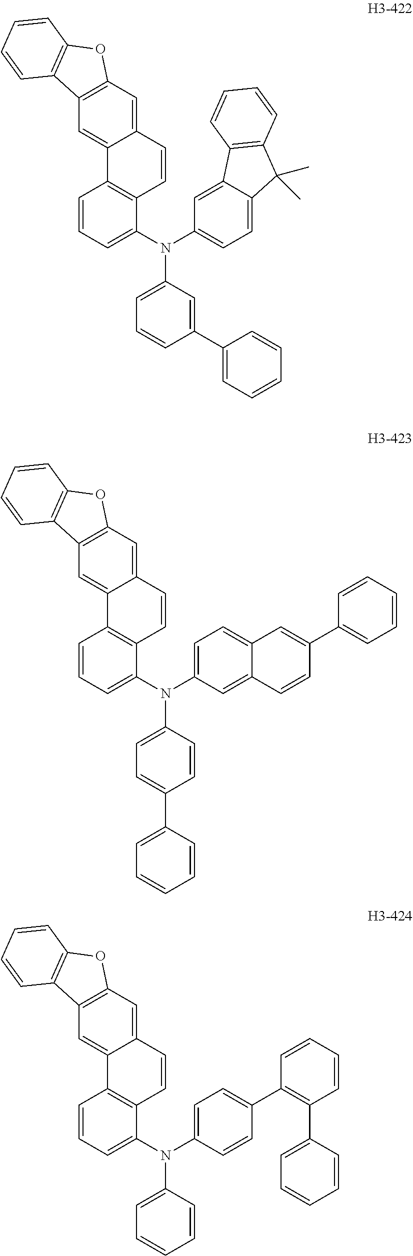 [SMILES: CC1(C)c2ccccc2-c2cc(N(c3cccc(-c4ccccc4)c3)c3cccc4c3ccc3cc5oc6ccccc6c5cc34)ccc21 ; c1ccc(-c2ccc(N(c3ccc4cc(-c5ccccc5)ccc4c3)c3cccc4c3ccc3cc5oc6ccccc6c5cc34)cc2)cc1 ; c1ccc(-c2ccccc2-c2ccc(N(c3ccccc3)c3cccc4c3ccc3cc5oc6ccccc6c5cc34)cc2)cc1]

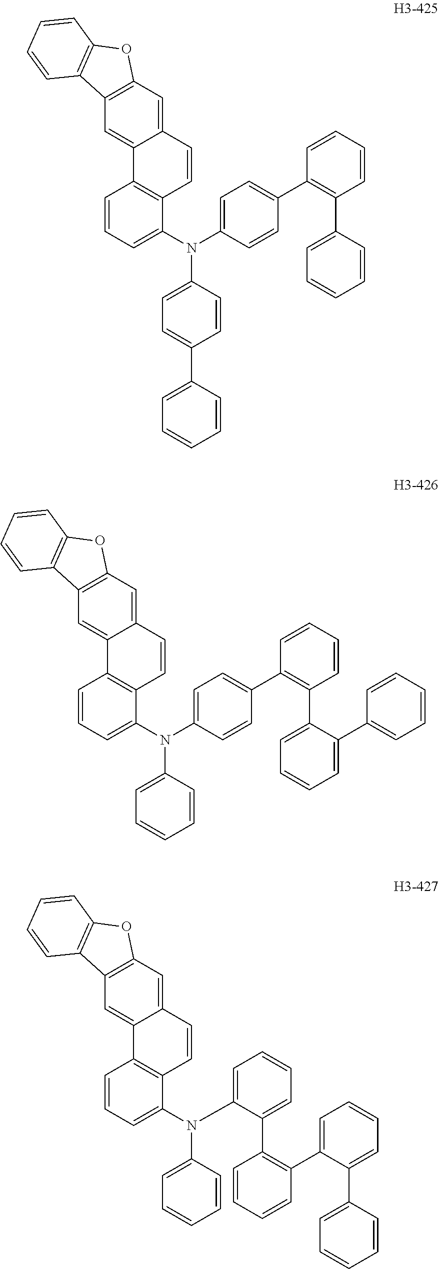 [SMILES: c1ccc(-c2ccc(N(c3ccc(-c4ccccc4-c4ccccc4)cc3)c3cccc4c3ccc3cc5oc6ccccc6c5cc34)cc2)cc1 ; c1ccc(-c2ccccc2-c2ccccc2-c2ccc(N(c3ccccc3)c3cccc4c3ccc3cc5oc6ccccc6c5cc34)cc2)cc1 ; c1ccc(-c2ccccc2-c2ccccc2-c2ccccc2N(c2ccccc2)c2cccc3c2ccc2cc4oc5ccccc5c4cc23)cc1]

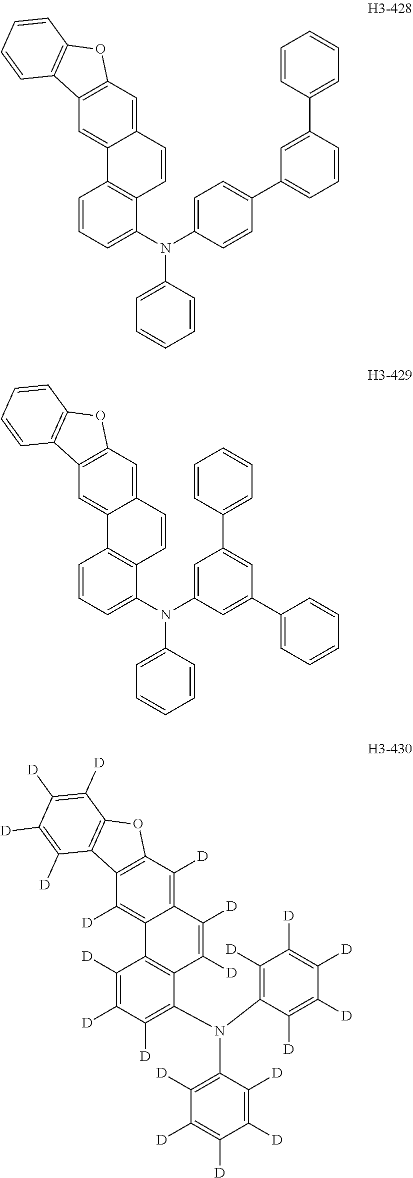 [SMILES: c1ccc(-c2cccc(-c3ccc(N(c4ccccc4)c4cccc5c4ccc4cc6oc7ccccc7c6cc45)cc3)c2)cc1 ; c1ccc(-c2cc(-c3ccccc3)cc(N(c3ccccc3)c3cccc4c3ccc3cc5oc6ccccc6c5cc34)c2)cc1 ; [2H]c1c([2H])c([2H])c(N(c2c([2H])c([2H])c([2H])c([2H])c2[2H])c2c([2H])c([2H])c([2H])c3c2c([2H])c([2H])c2c([2H])c4oc5c([2H])c([2H])c([2H])c([2H])c5c4c([2H])c23)c([2H])c1[2H]]

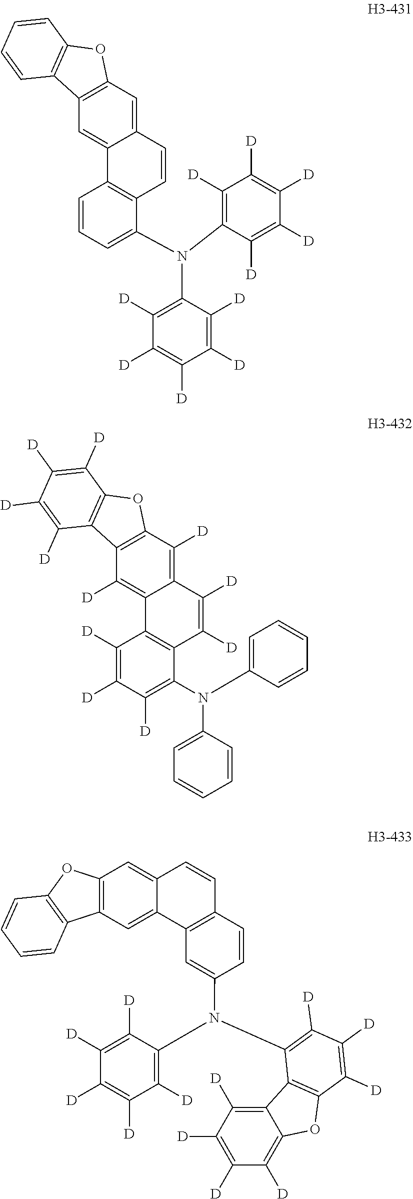 [SMILES: [2H]c1c([2H])c([2H])c(N(c2c([2H])c([2H])c([2H])c([2H])c2[2H])c2cccc3c2ccc2cc4oc5ccccc5c4cc23)c([2H])c1[2H] ; [2H]c1c([2H])c([2H])c2c(oc3c([2H])c4c([2H])c([2H])c5c(N(c6ccccc6)c6ccccc6)c([2H])c([2H])c([2H])c5c4c([2H])c32)c1[2H] ; [2H]c1c([2H])c([2H])c(N(c2ccc3ccc4cc5oc6ccccc6c5cc4c3c2)c2c([2H])c([2H])c([2H])c3oc4c([2H])c([2H])c([2H])c([2H])c4c23)c([2H])c1[2H]]

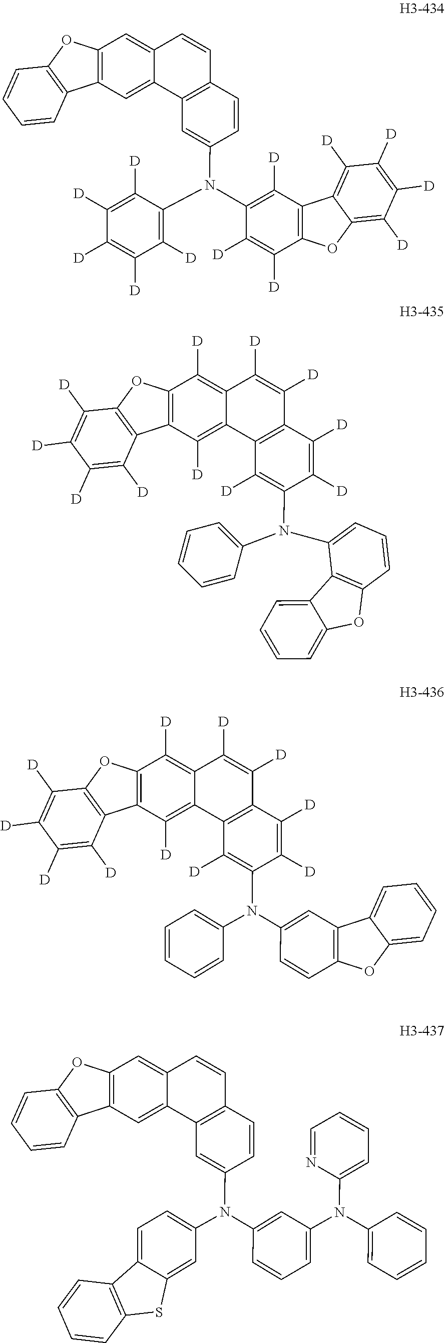 [SMILES: [2H]c1c([2H])c([2H])c(N(c2ccc3ccc4cc5oc6ccccc6c5cc4c3c2)c2c([2H])c([2H])c3oc4c([2H])c([2H])c([2H])c([2H])c4c3c2[2H])c([2H])c1[2H] ; [2H]c1c([2H])c([2H])c2c(oc3c([2H])c4c([2H])c([2H])c5c([2H])c([2H])c(N(c6ccccc6)c6cccc7oc8ccccc8c67)c([2H])c5c4c([2H])c32)c1[2H] ; [2H]c1c([2H])c([2H])c2c(oc3c([2H])c4c([2H])c([2H])c5c([2H])c([2H])c(N(c6ccccc6)c6ccc7oc8ccccc8c7c6)c([2H])c5c4c([2H])c32)c1[2H] ; c1ccc(N(c2cccc(N(c3ccc4c(c3)sc3ccccc34)c3ccc4ccc5cc6oc7ccccc7c6cc5c4c3)c2)c2ccccn2)cc1]

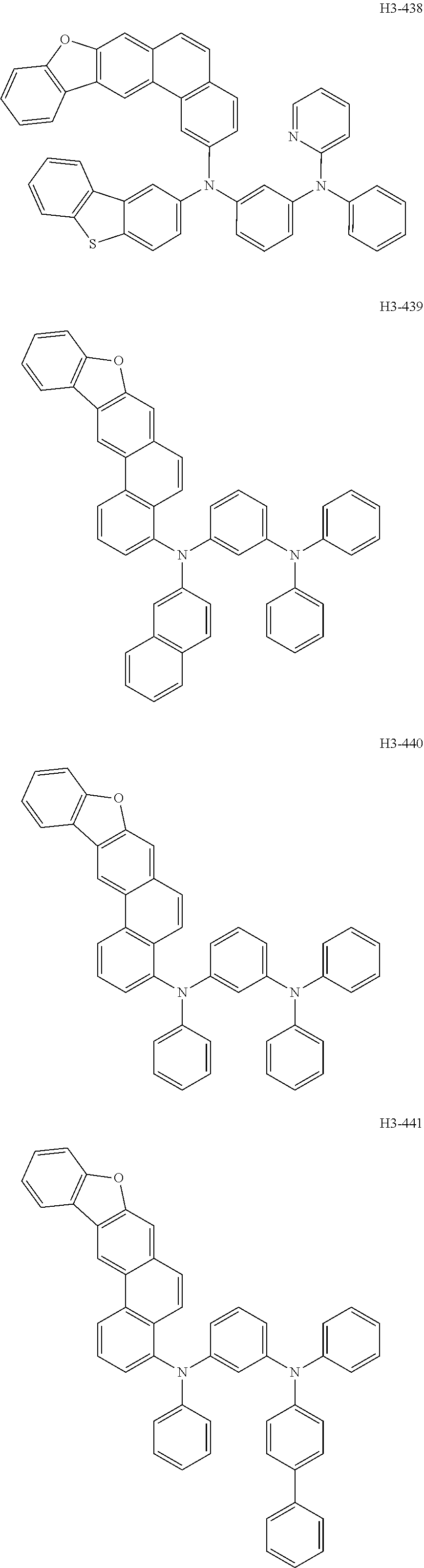 [SMILES: c1ccc(N(c2cccc(N(c3ccc4ccc5cc6oc7ccccc7c6cc5c4c3)c3ccc4sc5ccccc5c4c3)c2)c2ccccn2)cc1 ; c1ccc(N(c2ccccc2)c2cccc(N(c3ccc4ccccc4c3)c3cccc4c3ccc3cc5oc6ccccc6c5cc34)c2)cc1 ; c1ccc(N(c2ccccc2)c2cccc(N(c3ccccc3)c3cccc4c3ccc3cc5oc6ccccc6c5cc34)c2)cc1 ; c1ccc(-c2ccc(N(c3ccccc3)c3cccc(N(c4ccccc4)c4cccc5c4ccc4cc6oc7ccccc7c6cc45)c3)cc2)cc1]

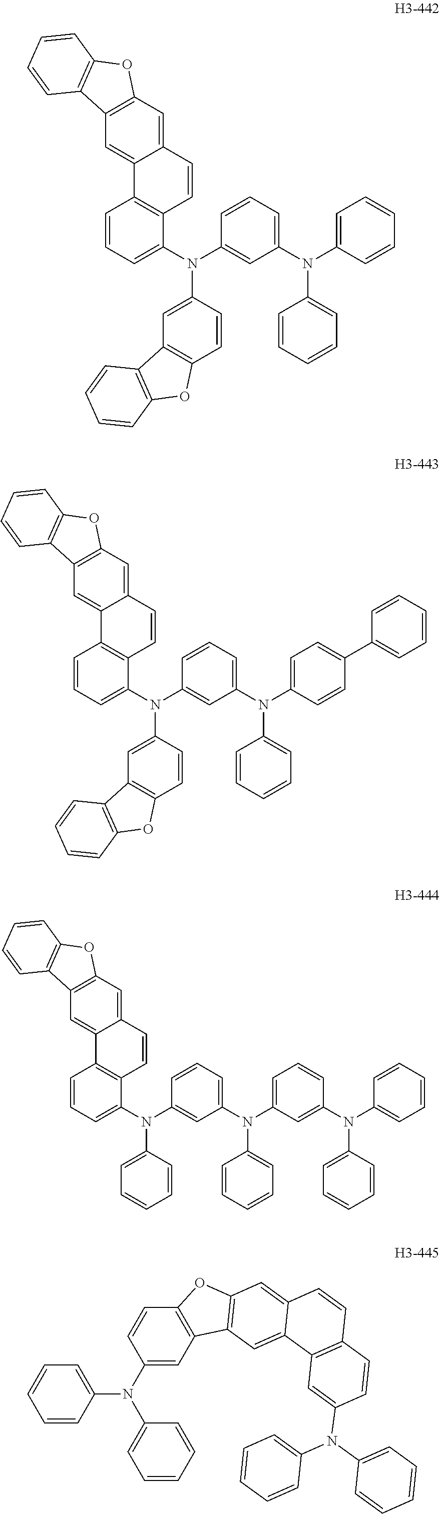 [SMILES: c1ccc(N(c2ccccc2)c2cccc(N(c3ccc4oc5ccccc5c4c3)c3cccc4c3ccc3cc5oc6ccccc6c5cc34)c2)cc1 ; c1ccc(-c2ccc(N(c3ccccc3)c3cccc(N(c4ccc5oc6ccccc6c5c4)c4cccc5c4ccc4cc6oc7ccccc7c6cc45)c3)cc2)cc1 ; c1ccc(N(c2ccccc2)c2cccc(N(c3ccccc3)c3cccc(N(c4ccccc4)c4cccc5c4ccc4cc6oc7ccccc7c6cc45)c3)c2)cc1 ; c1ccc(N(c2ccccc2)c2ccc3ccc4cc5oc6ccc(N(c7ccccc7)c7ccccc7)cc6c5cc4c3c2)cc1]

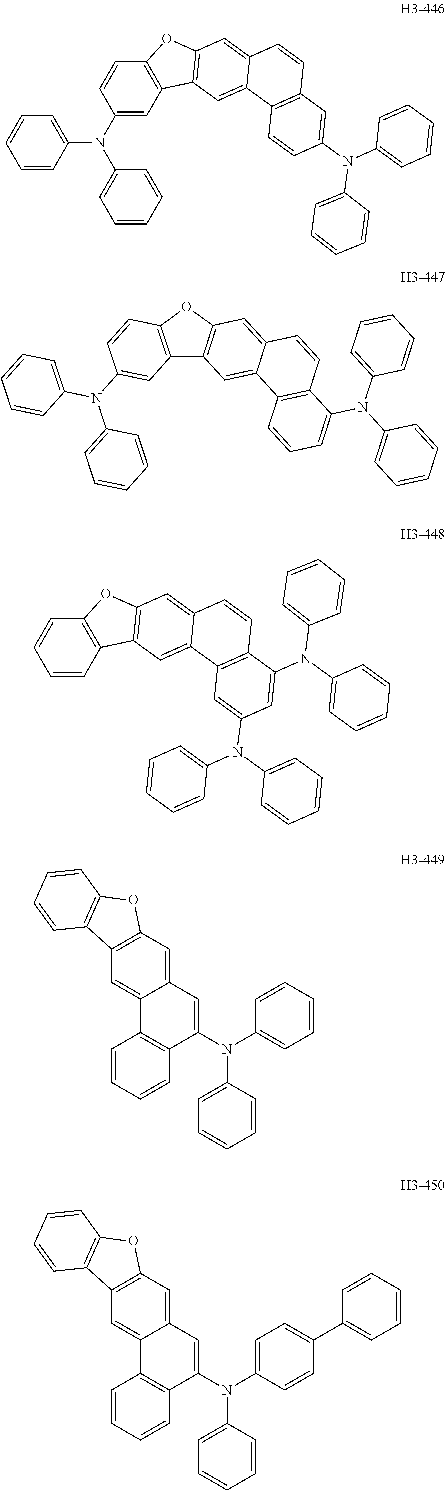 [SMILES: c1ccc(N(c2ccccc2)c2ccc3c(ccc4cc5oc6ccc(N(c7ccccc7)c7ccccc7)cc6c5cc43)c2)cc1 ; c1ccc(N(c2ccccc2)c2ccc3oc4cc5ccc6c(N(c7ccccc7)c7ccccc7)cccc6c5cc4c3c2)cc1 ; c1ccc(N(c2ccccc2)c2cc(N(c3ccccc3)c3ccccc3)c3ccc4cc5oc6ccccc6c5cc4c3c2)cc1 ; c1ccc(N(c2ccccc2)c2cc3cc4oc5ccccc5c4cc3c3ccccc23)cc1 ; c1ccc(-c2ccc(N(c3ccccc3)c3cc4cc5oc6ccccc6c5cc4c4ccccc34)cc2)cc1]

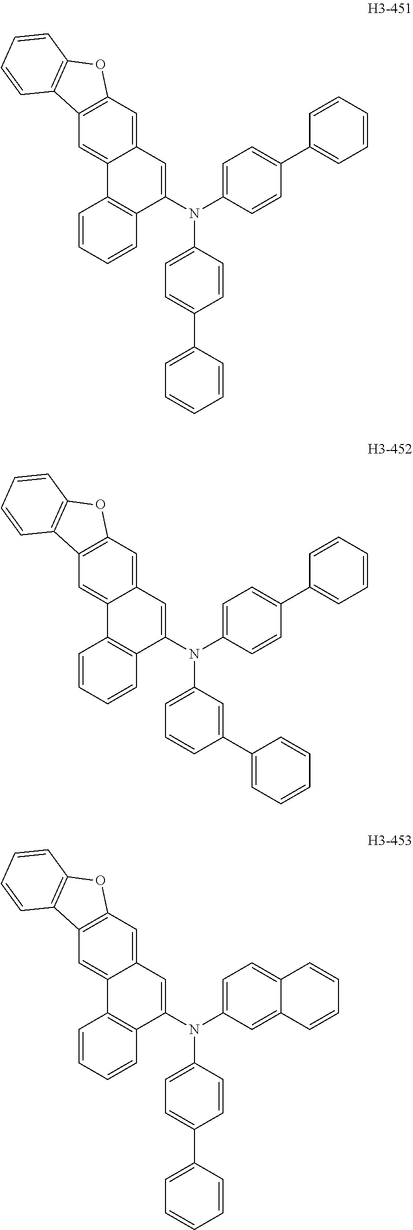 [SMILES: c1ccc(-c2ccc(N(c3ccc(-c4ccccc4)cc3)c3cc4cc5oc6ccccc6c5cc4c4ccccc34)cc2)cc1 ; c1ccc(-c2ccc(N(c3cccc(-c4ccccc4)c3)c3cc4cc5oc6ccccc6c5cc4c4ccccc34)cc2)cc1 ; c1ccc(-c2ccc(N(c3ccc4ccccc4c3)c3cc4cc5oc6ccccc6c5cc4c4ccccc34)cc2)cc1]

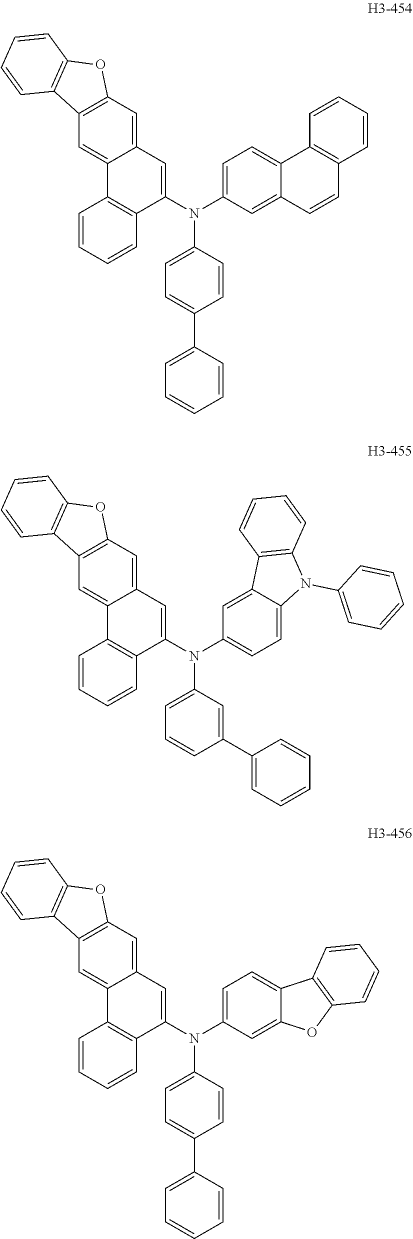 [SMILES: c1ccc(-c2ccc(N(c3ccc4c(ccc5ccccc54)c3)c3cc4cc5oc6ccccc6c5cc4c4ccccc34)cc2)cc1 ; c1ccc(-c2cccc(N(c3ccc4c(c3)c3ccccc3n4-c3ccccc3)c3cc4cc5oc6ccccc6c5cc4c4ccccc34)c2)cc1 ; c1ccc(-c2ccc(N(c3ccc4c(c3)oc3ccccc34)c3cc4cc5oc6ccccc6c5cc4c4ccccc34)cc2)cc1]

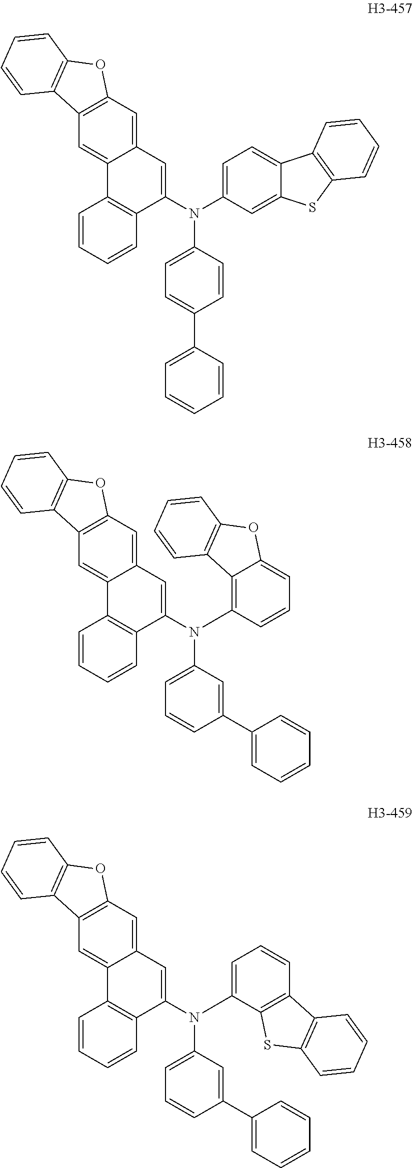 [SMILES: c1ccc(-c2ccc(N(c3ccc4c(c3)sc3ccccc34)c3cc4cc5oc6ccccc6c5cc4c4ccccc34)cc2)cc1 ; c1ccc(-c2cccc(N(c3cc4cc5oc6ccccc6c5cc4c4ccccc34)c3cccc4oc5ccccc5c34)c2)cc1 ; c1ccc(-c2cccc(N(c3cc4cc5oc6ccccc6c5cc4c4ccccc34)c3cccc4c3sc3ccccc34)c2)cc1]

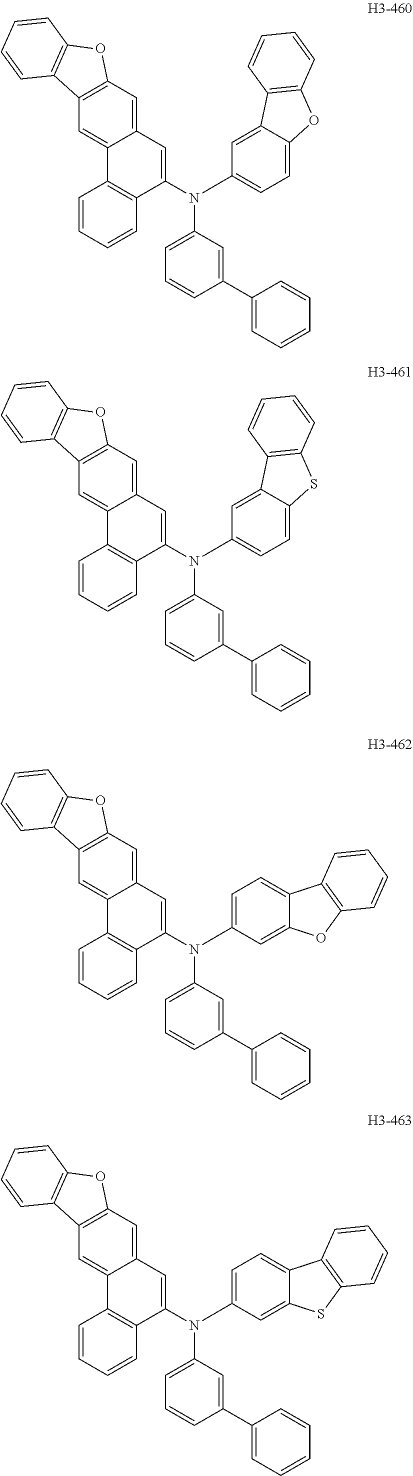 [SMILES: c1ccc(-c2cccc(N(c3ccc4oc5ccccc5c4c3)c3cc4cc5oc6ccccc6c5cc4c4ccccc34)c2)cc1 ; c1ccc(-c2cccc(N(c3ccc4sc5ccccc5c4c3)c3cc4cc5oc6ccccc6c5cc4c4ccccc34)c2)cc1 ; c1ccc(-c2cccc(N(c3ccc4c(c3)oc3ccccc34)c3cc4cc5oc6ccccc6c5cc4c4ccccc34)c2)cc1 ; c1ccc(-c2cccc(N(c3ccc4c(c3)sc3ccccc34)c3cc4cc5oc6ccccc6c5cc4c4ccccc34)c2)cc1]

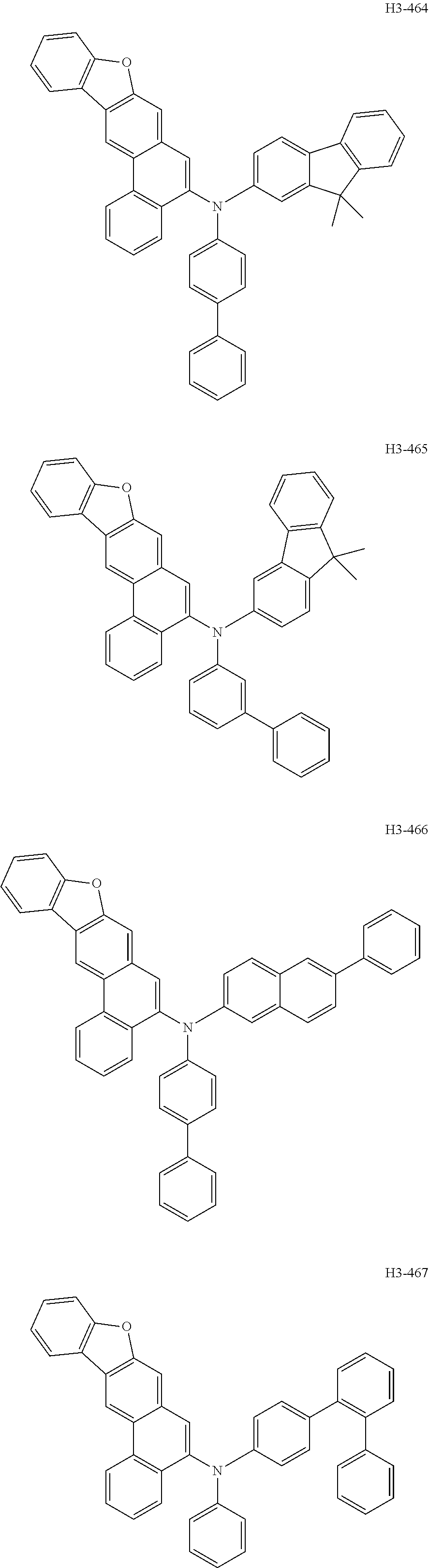 [SMILES: CC1(C)c2ccccc2-c2ccc(N(c3ccc(-c4ccccc4)cc3)c3cc4cc5oc6ccccc6c5cc4c4ccccc34)cc21 ; CC1(C)c2ccccc2-c2cc(N(c3cccc(-c4ccccc4)c3)c3cc4cc5oc6ccccc6c5cc4c4ccccc34)ccc21 ; c1ccc(-c2ccc(N(c3ccc4cc(-c5ccccc5)ccc4c3)c3cc4cc5oc6ccccc6c5cc4c4ccccc34)cc2)cc1 ; c1ccc(-c2ccccc2-c2ccc(N(c3ccccc3)c3cc4cc5oc6ccccc6c5cc4c4ccccc34)cc2)cc1]

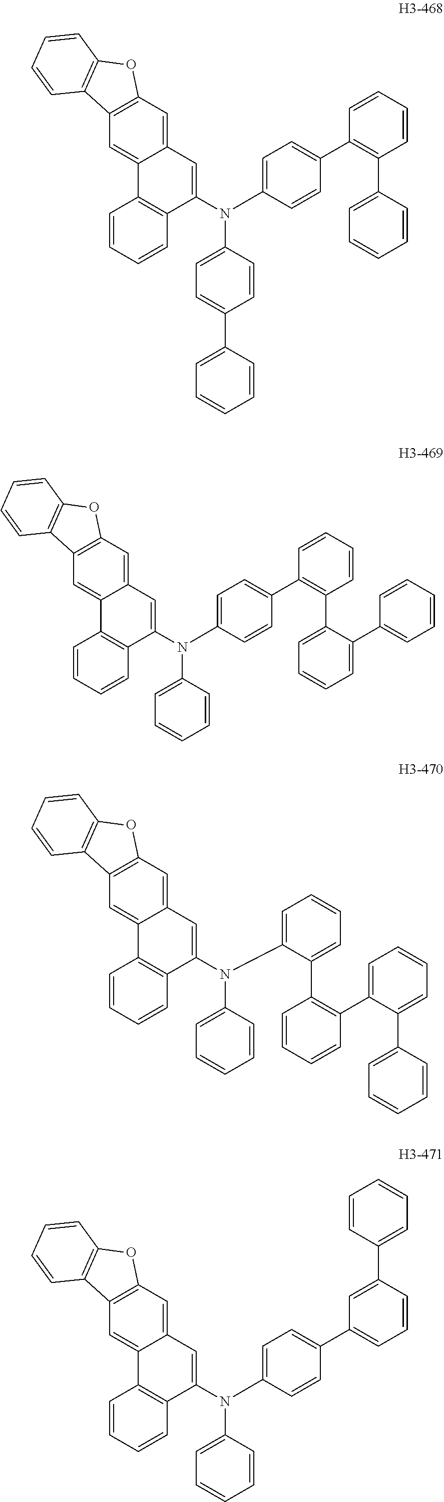 [SMILES: c1ccc(-c2ccc(N(c3ccc(-c4ccccc4-c4ccccc4)cc3)c3cc4cc5oc6ccccc6c5cc4c4ccccc34)cc2)cc1 ; c1ccc(-c2ccccc2-c2ccccc2-c2ccc(N(c3ccccc3)c3cc4cc5oc6ccccc6c5cc4c4ccccc34)cc2)cc1 ; c1ccc(-c2ccccc2-c2ccccc2-c2ccccc2N(c2ccccc2)c2cc3cc4oc5ccccc5c4cc3c3ccccc23)cc1 ; c1ccc(-c2cccc(-c3ccc(N(c4ccccc4)c4cc5cc6oc7ccccc7c6cc5c5ccccc45)cc3)c2)cc1]

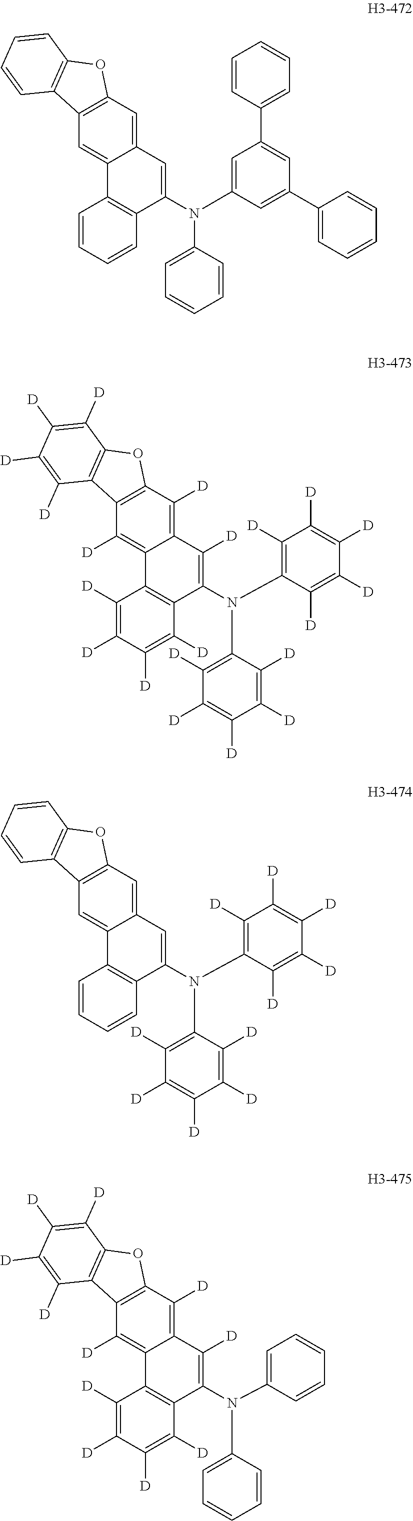 [SMILES: c1ccc(-c2cc(-c3ccccc3)cc(N(c3ccccc3)c3cc4cc5oc6ccccc6c5cc4c4ccccc34)c2)cc1 ; [2H]c1c([2H])c([2H])c(N(c2c([2H])c([2H])c([2H])c([2H])c2[2H])c2c([2H])c3c([2H])c4oc5c([2H])c([2H])c([2H])c([2H])c5c4c([2H])c3c3c([2H])c([2H])c([2H])c([2H])c23)c([2H])c1[2H] ; [2H]c1c([2H])c([2H])c(N(c2c([2H])c([2H])c([2H])c([2H])c2[2H])c2cc3cc4oc5ccccc5c4cc3c3ccccc23)c([2H])c1[2H] ; [2H]c1c([2H])c([2H])c2c(oc3c([2H])c4c([2H])c(N(c5ccccc5)c5ccccc5)c5c([2H])c([2H])c([2H])c([2H])c5c4c([2H])c32)c1[2H]]

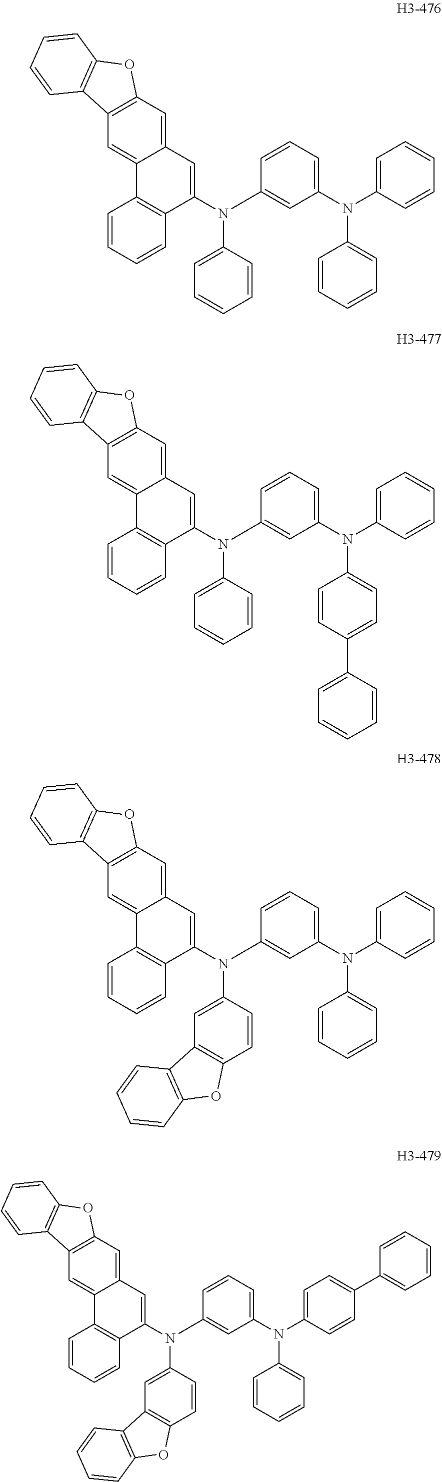 [SMILES: c1ccc(N(c2ccccc2)c2cccc(N(c3ccccc3)c3cc4cc5oc6ccccc6c5cc4c4ccccc34)c2)cc1 ; c1ccc(-c2ccc(N(c3ccccc3)c3cccc(N(c4ccccc4)c4cc5cc6oc7ccccc7c6cc5c5ccccc45)c3)cc2)cc1 ; c1ccc(N(c2ccccc2)c2cccc(N(c3ccc4oc5ccccc5c4c3)c3cc4cc5oc6ccccc6c5cc4c4ccccc34)c2)cc1 ; c1ccc(-c2ccc(N(c3ccccc3)c3cccc(N(c4ccc5oc6ccccc6c5c4)c4cc5cc6oc7ccccc7c6cc5c5ccccc45)c3)cc2)cc1]

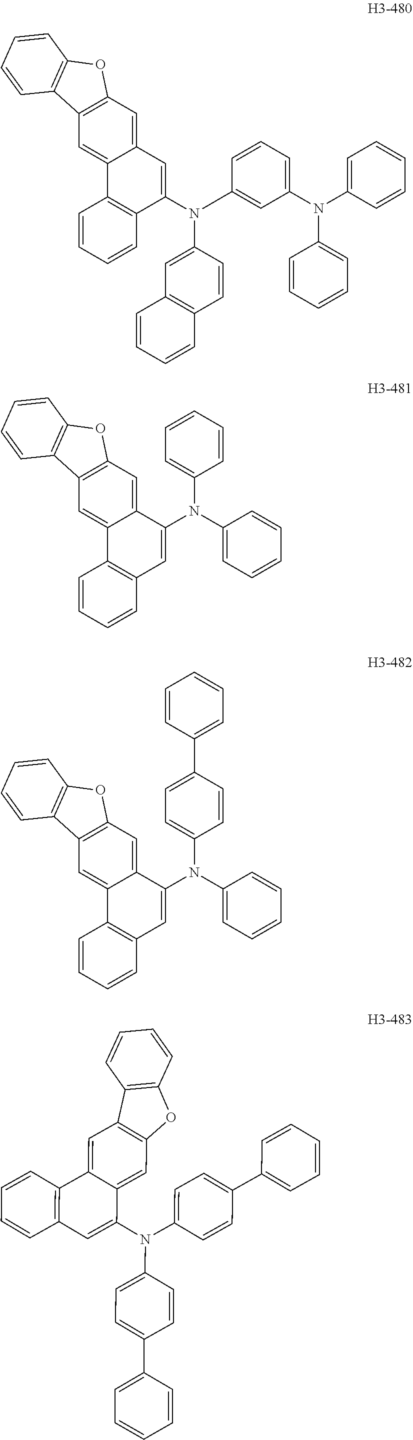

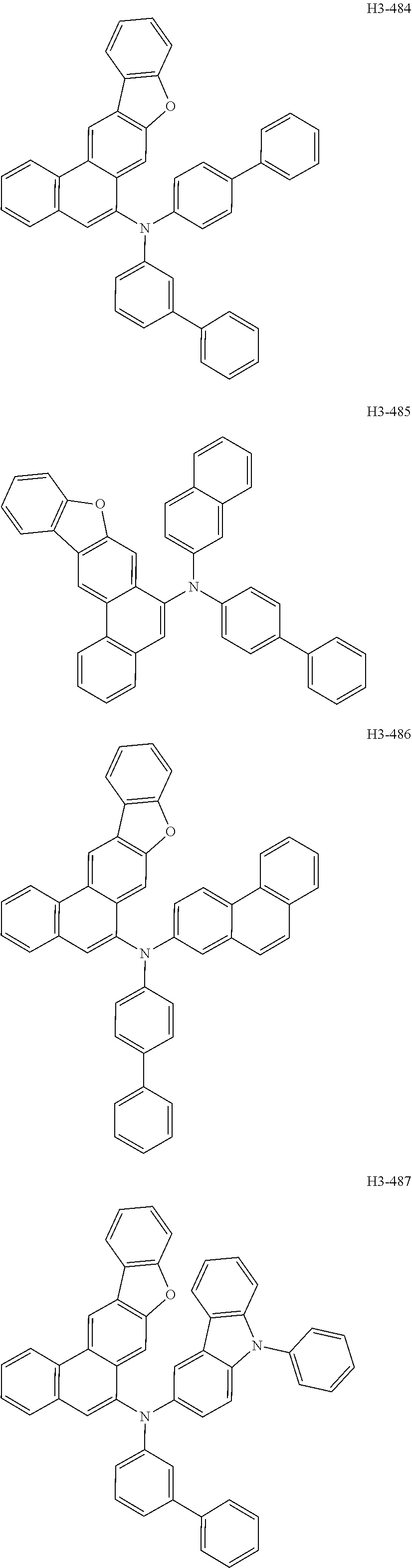

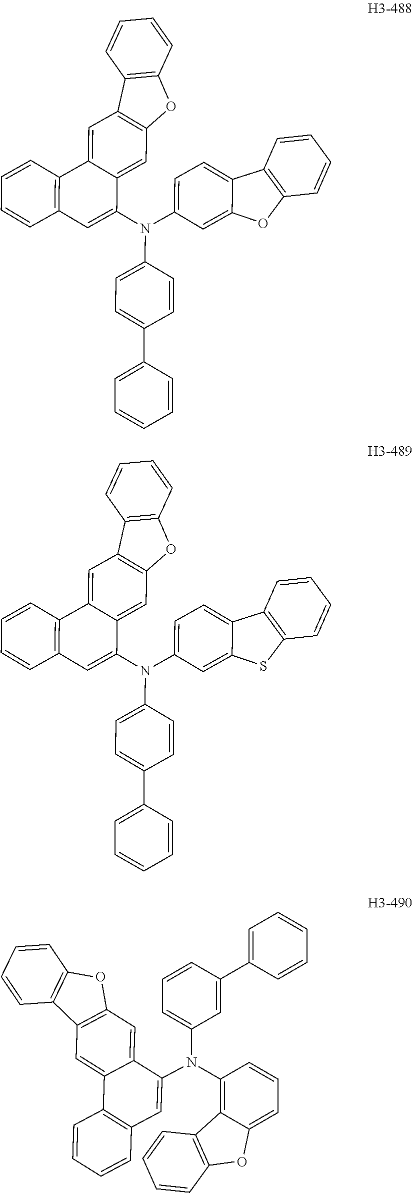

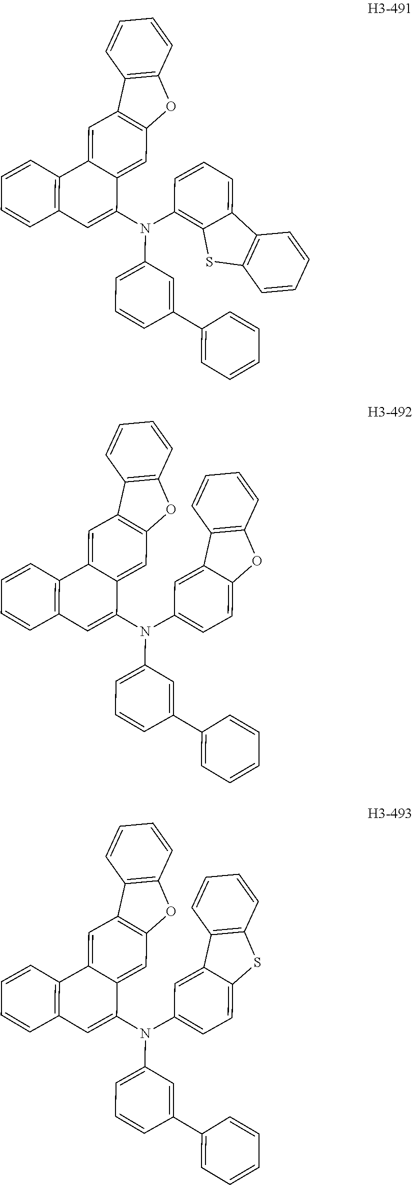 [SMILES: c1ccc(-c2cccc(N(c3cc4ccccc4c4cc5c(cc34)oc3ccccc35)c3cccc4c3sc3ccccc34)c2)cc1 ; c1ccc(-c2cccc(N(c3ccc4oc5ccccc5c4c3)c3cc4ccccc4c4cc5c(cc34)oc3ccccc35)c2)cc1 ; c1ccc(-c2cccc(N(c3ccc4sc5ccccc5c4c3)c3cc4ccccc4c4cc5c(cc34)oc3ccccc35)c2)cc1]

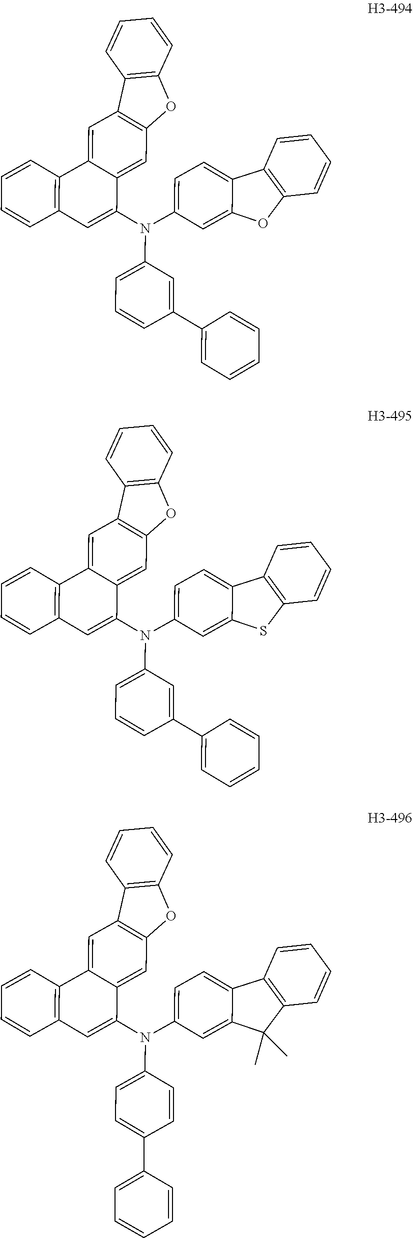 [SMILES: c1ccc(-c2cccc(N(c3ccc4c(c3)oc3ccccc34)c3cc4ccccc4c4cc5c(cc34)oc3ccccc35)c2)cc1 ; c1ccc(-c2cccc(N(c3ccc4c(c3)sc3ccccc34)c3cc4ccccc4c4cc5c(cc34)oc3ccccc35)c2)cc1 ; CC1(C)c2ccccc2-c2ccc(N(c3ccc(-c4ccccc4)cc3)c3cc4ccccc4c4cc5c(cc34)oc3ccccc35)cc21]

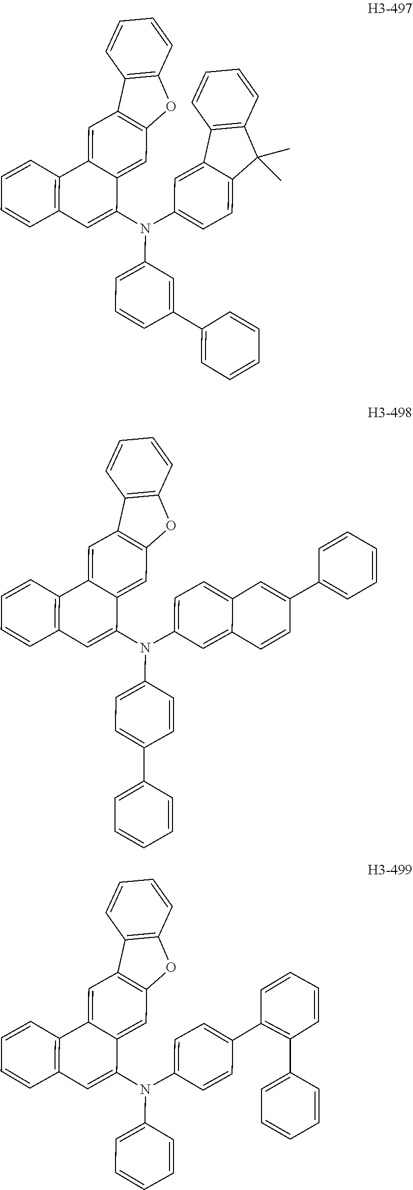

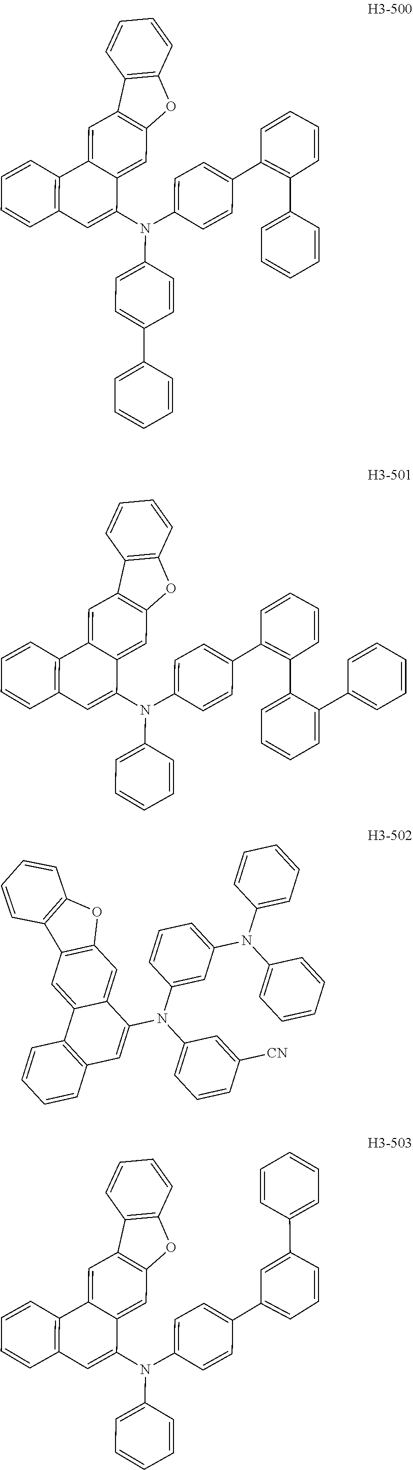 [SMILES: c1ccc(-c2ccc(N(c3ccc(-c4ccccc4-c4ccccc4)cc3)c3cc4ccccc4c4cc5c(cc34)oc3ccccc35)cc2)cc1 ; c1ccc(-c2ccccc2-c2ccccc2-c2ccc(N(c3ccccc3)c3cc4ccccc4c4cc5c(cc34)oc3ccccc35)cc2)cc1 ; N#Cc1cccc(N(c2cccc(N(c3ccccc3)c3ccccc3)c2)c2cc3ccccc3c3cc4c(cc23)oc2ccccc24)c1 ; c1ccc(-c2cccc(-c3ccc(N(c4ccccc4)c4cc5ccccc5c5cc6c(cc45)oc4ccccc46)cc3)c2)cc1]

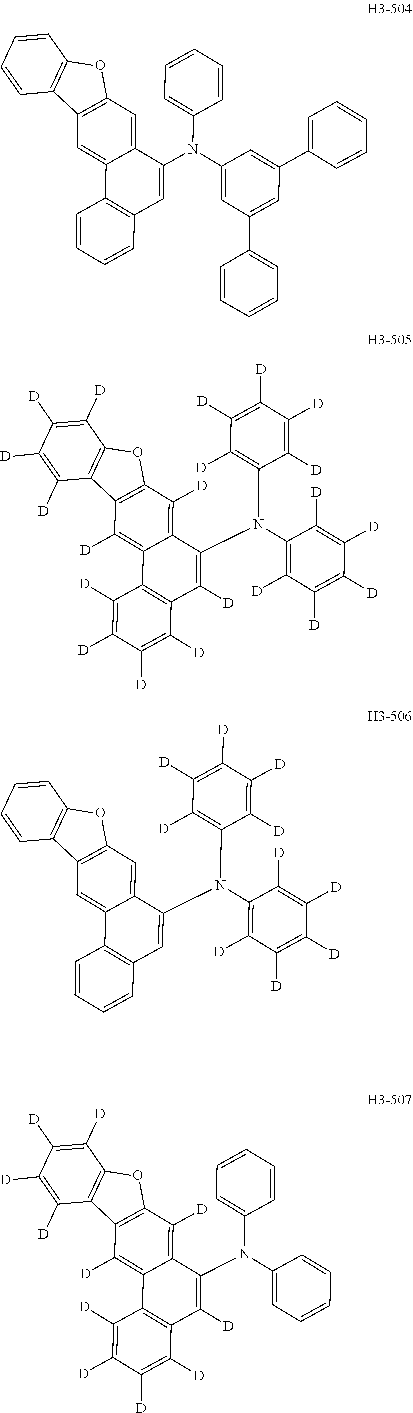 [SMILES: c1ccc(-c2cc(-c3ccccc3)cc(N(c3ccccc3)c3cc4ccccc4c4cc5c(cc34)oc3ccccc35)c2)cc1 ; [2H]c1c([2H])c([2H])c(N(c2c([2H])c([2H])c([2H])c([2H])c2[2H])c2c([2H])c3c([2H])c([2H])c([2H])c([2H])c3c3c([2H])c4c(oc5c([2H])c([2H])c([2H])c([2H])c54)c([2H])c23)c([2H])c1[2H] ; [2H]c1c([2H])c([2H])c(N(c2c([2H])c([2H])c([2H])c([2H])c2[2H])c2cc3ccccc3c3cc4c(cc23)oc2ccccc24)c([2H])c1[2H] ; [2H]c1c([2H])c([2H])c2c(oc3c([2H])c4c(N(c5ccccc5)c5ccccc5)c([2H])c5c([2H])c([2H])c([2H])c([2H])c5c4c([2H])c32)c1[2H]]

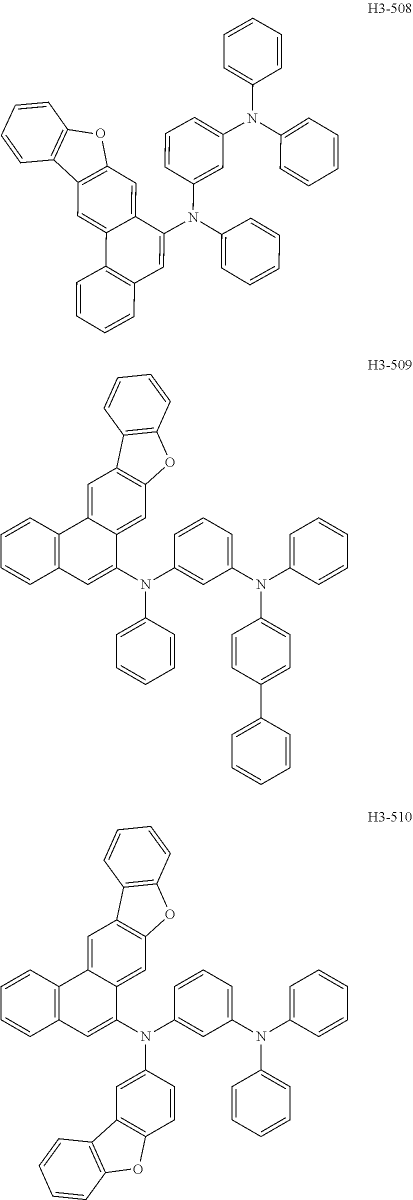 [SMILES: c1ccc(N(c2ccccc2)c2cccc(N(c3ccccc3)c3cc4ccccc4c4cc5c(cc34)oc3ccccc35)c2)cc1 ; c1ccc(-c2ccc(N(c3ccccc3)c3cccc(N(c4ccccc4)c4cc5ccccc5c5cc6c(cc45)oc4ccccc46)c3)cc2)cc1 ; c1ccc(N(c2ccccc2)c2cccc(N(c3ccc4oc5ccccc5c4c3)c3cc4ccccc4c4cc5c(cc34)oc3ccccc35)c2)cc1]

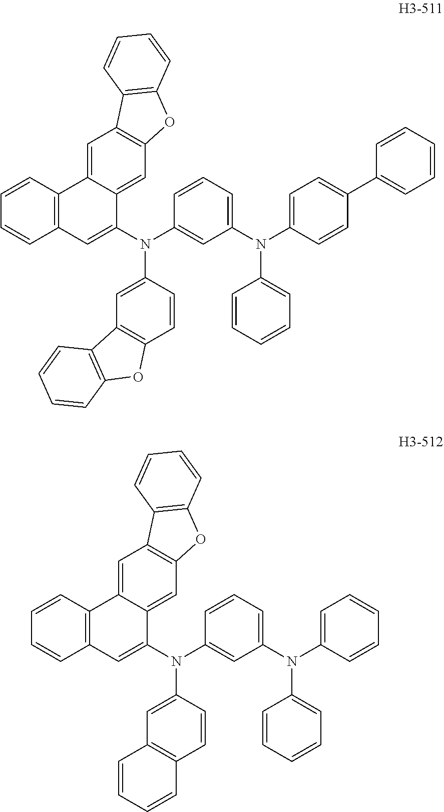 [SMILES: c1ccc(-c2ccc(N(c3ccccc3)c3cccc(N(c4ccc5oc6ccccc6c5c4)c4cc5ccccc5c5cc6c(cc45)oc4ccccc46)c3)cc2)cc1 ; c1ccc(N(c2ccccc2)c2cccc(N(c3ccc4ccccc4c3)c3cc4ccccc4c4cc5c(cc34)oc3ccccc35)c2)cc1]

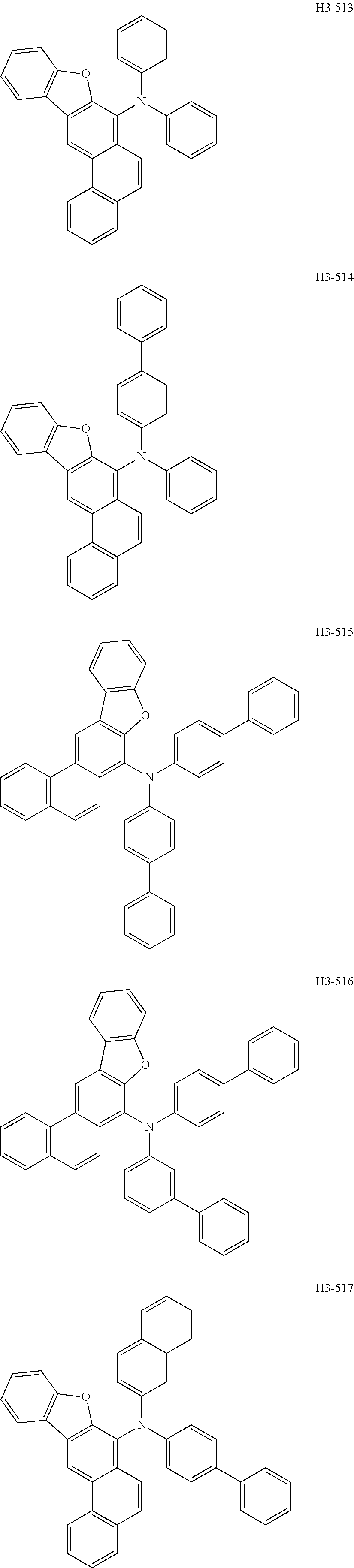 [SMILES: c1ccc(N(c2ccccc2)c2c3ccc4ccccc4c3cc3c2oc2ccccc23)cc1 ; c1ccc(-c2ccc(N(c3ccccc3)c3c4ccc5ccccc5c4cc4c3oc3ccccc34)cc2)cc1 ; c1ccc(-c2ccc(N(c3ccc(-c4ccccc4)cc3)c3c4ccc5ccccc5c4cc4c3oc3ccccc34)cc2)cc1 ; c1ccc(-c2ccc(N(c3cccc(-c4ccccc4)c3)c3c4ccc5ccccc5c4cc4c3oc3ccccc34)cc2)cc1 ; c1ccc(-c2ccc(N(c3ccc4ccccc4c3)c3c4ccc5ccccc5c4cc4c3oc3ccccc34)cc2)cc1]

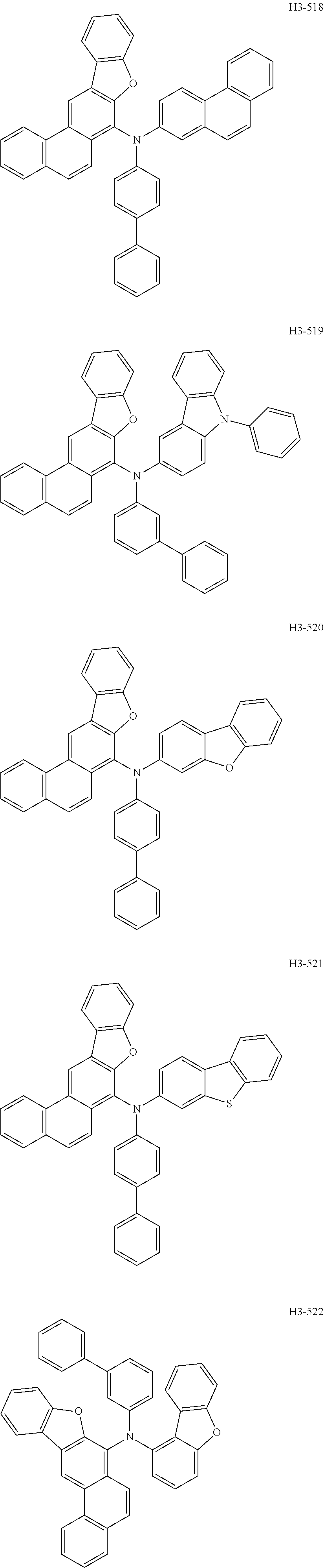 [SMILES: c1ccc(-c2ccc(N(c3ccc4c(ccc5ccccc54)c3)c3c4ccc5ccccc5c4cc4c3oc3ccccc34)cc2)cc1 ; c1ccc(-c2cccc(N(c3ccc4c(c3)c3ccccc3n4-c3ccccc3)c3c4ccc5ccccc5c4cc4c3oc3ccccc34)c2)cc1 ; c1ccc(-c2ccc(N(c3ccc4c(c3)oc3ccccc34)c3c4ccc5ccccc5c4cc4c3oc3ccccc34)cc2)cc1 ; c1ccc(-c2ccc(N(c3ccc4c(c3)sc3ccccc34)c3c4ccc5ccccc5c4cc4c3oc3ccccc34)cc2)cc1 ; c1ccc(-c2cccc(N(c3c4ccc5ccccc5c4cc4c3oc3ccccc34)c3cccc4oc5ccccc5c34)c2)cc1]

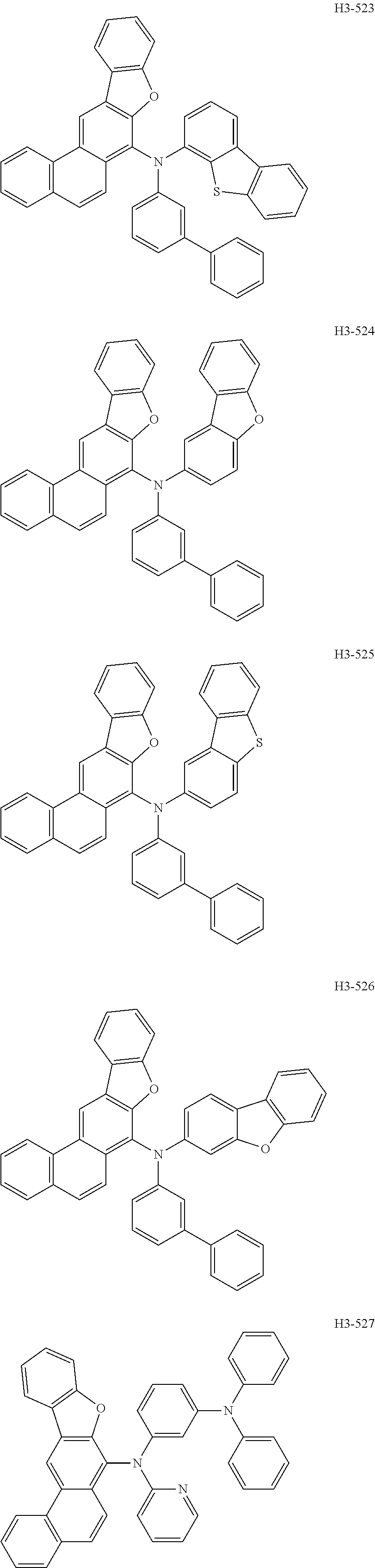

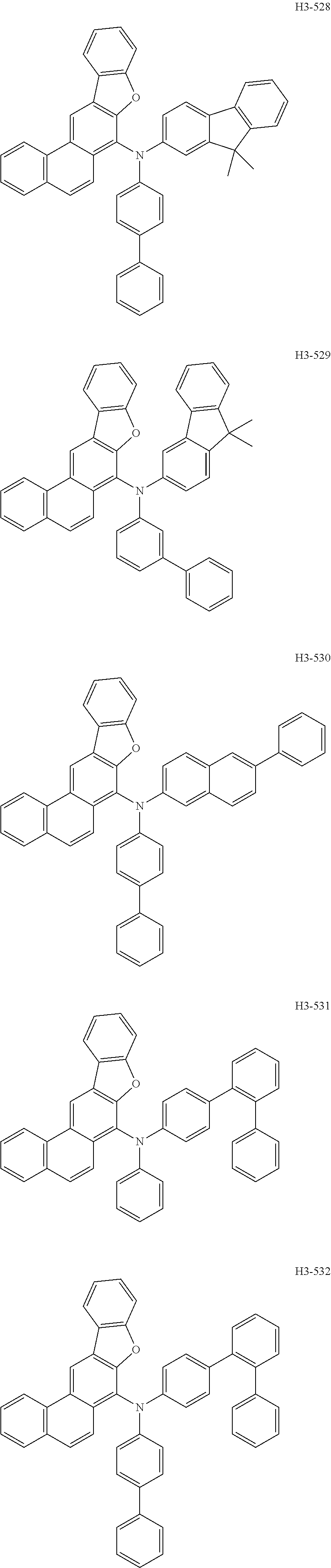 [SMILES: CC1(C)c2ccccc2-c2ccc(N(c3ccc(-c4ccccc4)cc3)c3c4ccc5ccccc5c4cc4c3oc3ccccc34)cc21 ; CC1(C)c2ccccc2-c2cc(N(c3cccc(-c4ccccc4)c3)c3c4ccc5ccccc5c4cc4c3oc3ccccc34)ccc21 ; c1ccc(-c2ccc(N(c3ccc4cc(-c5ccccc5)ccc4c3)c3c4ccc5ccccc5c4cc4c3oc3ccccc34)cc2)cc1 ; c1ccc(-c2ccccc2-c2ccc(N(c3ccccc3)c3c4ccc5ccccc5c4cc4c3oc3ccccc34)cc2)cc1 ; c1ccc(-c2ccc(N(c3ccc(-c4ccccc4-c4ccccc4)cc3)c3c4ccc5ccccc5c4cc4c3oc3ccccc34)cc2)cc1]

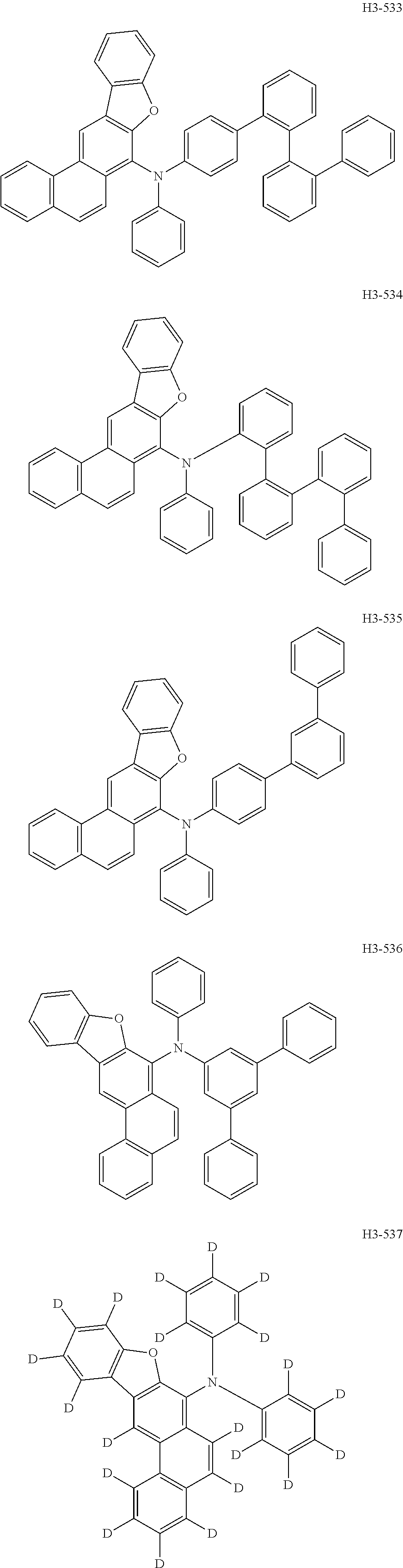 [SMILES: c1ccc(-c2ccccc2-c2ccccc2-c2ccc(N(c3ccccc3)c3c4ccc5ccccc5c4cc4c3oc3ccccc34)cc2)cc1 ; c1ccc(-c2ccccc2-c2ccccc2-c2ccccc2N(c2ccccc2)c2c3ccc4ccccc4c3cc3c2oc2ccccc23)cc1 ; c1ccc(-c2cccc(-c3ccc(N(c4ccccc4)c4c5ccc6ccccc6c5cc5c4oc4ccccc45)cc3)c2)cc1 ; c1ccc(-c2cc(-c3ccccc3)cc(N(c3ccccc3)c3c4ccc5ccccc5c4cc4c3oc3ccccc34)c2)cc1 ; [2H]c1c([2H])c([2H])c(N(c2c([2H])c([2H])c([2H])c([2H])c2[2H])c2c3oc4c([2H])c([2H])c([2H])c([2H])c4c3c([2H])c3c2c([2H])c([2H])c2c([2H])c([2H])c([2H])c([2H])c23)c([2H])c1[2H]]

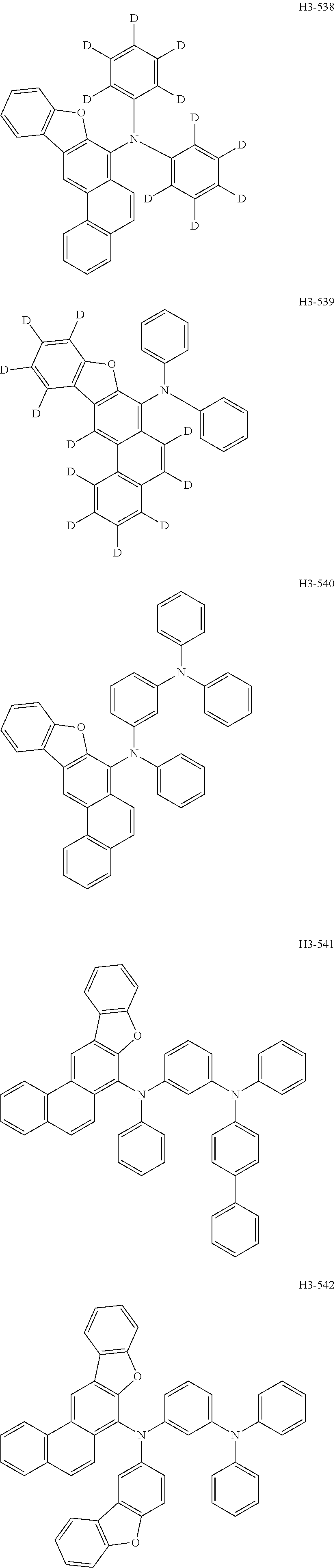 [SMILES: [2H]c1c([2H])c([2H])c(N(c2c([2H])c([2H])c([2H])c([2H])c2[2H])c2c3ccc4ccccc4c3cc3c2oc2ccccc23)c([2H])c1[2H] ; [2H]c1c([2H])c([2H])c2c(oc3c(N(c4ccccc4)c4ccccc4)c4c([2H])c([2H])c5c([2H])c([2H])c([2H])c([2H])c5c4c([2H])c32)c1[2H] ; c1ccc(N(c2ccccc2)c2cccc(N(c3ccccc3)c3c4ccc5ccccc5c4cc4c3oc3ccccc34)c2)cc1 ; c1ccc(-c2ccc(N(c3ccccc3)c3cccc(N(c4ccccc4)c4c5ccc6ccccc6c5cc5c4oc4ccccc45)c3)cc2)cc1 ; c1ccc(N(c2ccccc2)c2cccc(N(c3ccc4oc5ccccc5c4c3)c3c4ccc5ccccc5c4cc4c3oc3ccccc34)c2)cc1]

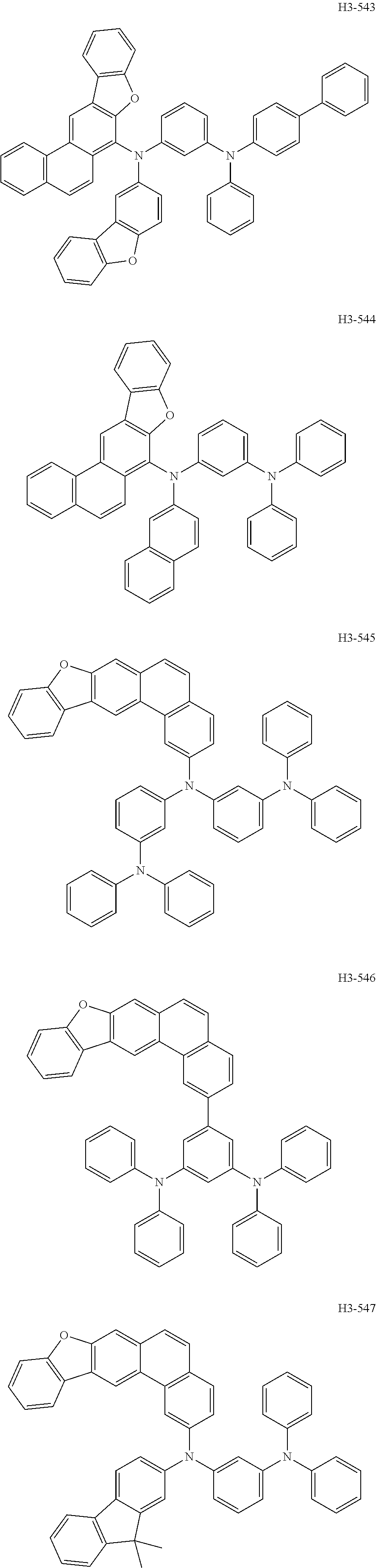 [SMILES: c1ccc(-c2ccc(N(c3ccccc3)c3cccc(N(c4ccc5oc6ccccc6c5c4)c4c5ccc6ccccc6c5cc5c4oc4ccccc45)c3)cc2)cc1 ; c1ccc(N(c2ccccc2)c2cccc(N(c3ccc4ccccc4c3)c3c4ccc5ccccc5c4cc4c3oc3ccccc34)c2)cc1 ; c1ccc(N(c2ccccc2)c2cccc(N(c3cccc(N(c4ccccc4)c4ccccc4)c3)c3ccc4ccc5cc6oc7ccccc7c6cc5c4c3)c2)cc1 ; c1ccc(N(c2ccccc2)c2cc(-c3ccc4ccc5cc6oc7ccccc7c6cc5c4c3)cc(N(c3ccccc3)c3ccccc3)c2)cc1 ; CC1(C)c2ccccc2-c2ccc(N(c3cccc(N(c4ccccc4)c4ccccc4)c3)c3ccc4ccc5cc6oc7ccccc7c6cc5c4c3)cc21]

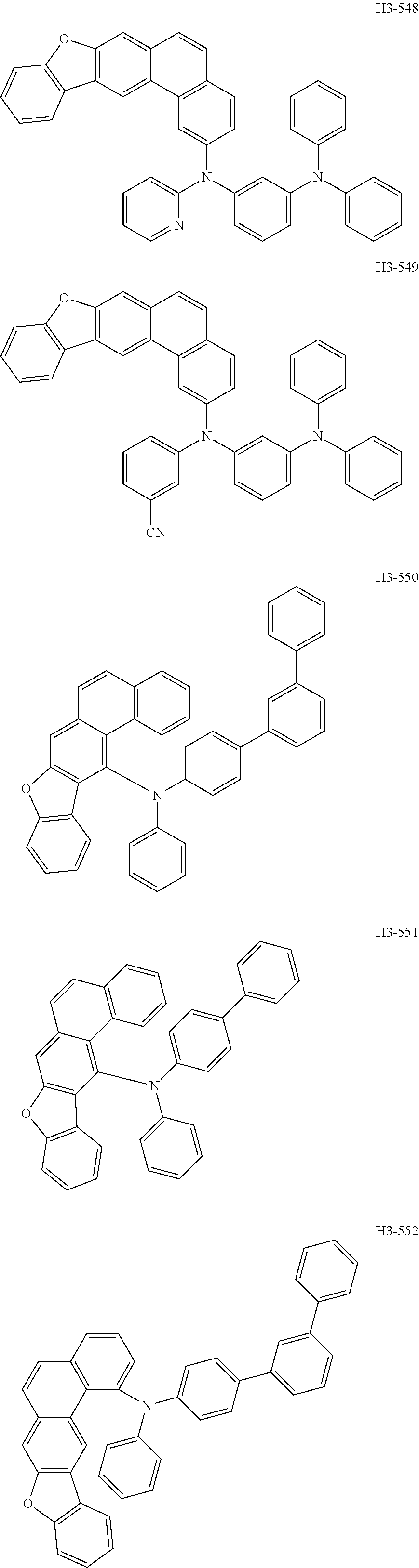 [SMILES: c1ccc(N(c2ccccc2)c2cccc(N(c3ccc4ccc5cc6oc7ccccc7c6cc5c4c3)c3ccccn3)c2)cc1 ; N#Cc1cccc(N(c2cccc(N(c3ccccc3)c3ccccc3)c2)c2ccc3ccc4cc5oc6ccccc6c5cc4c3c2)c1 ; c1ccc(-c2cccc(-c3ccc(N(c4ccccc4)c4c5c(ccc6ccccc65)cc5oc6ccccc6c45)cc3)c2)cc1 ; c1ccc(-c2ccc(N(c3ccccc3)c3c4c(ccc5ccccc54)cc4oc5ccccc5c34)cc2)cc1 ; c1ccc(-c2cccc(-c3ccc(N(c4ccccc4)c4cccc5ccc6cc7oc8ccccc8c7cc6c45)cc3)c2)cc1]

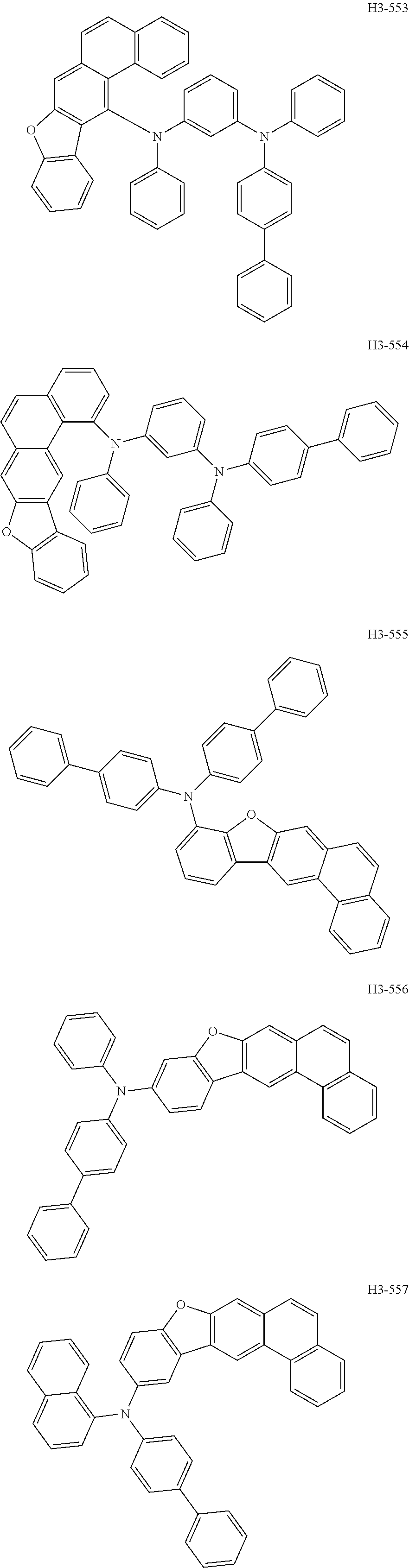 [SMILES: c1ccc(-c2ccc(N(c3ccccc3)c3cccc(N(c4ccccc4)c4c5c(ccc6ccccc65)cc5oc6ccccc6c45)c3)cc2)cc1 ; c1ccc(-c2ccc(N(c3ccccc3)c3cccc(N(c4ccccc4)c4cccc5ccc6cc7oc8ccccc8c7cc6c45)c3)cc2)cc1 ; c1ccc(-c2ccc(N(c3ccc(-c4ccccc4)cc3)c3cccc4c3oc3cc5ccc6ccccc6c5cc34)cc2)cc1 ; c1ccc(-c2ccc(N(c3ccccc3)c3ccc4c(c3)oc3cc5ccc6ccccc6c5cc34)cc2)cc1 ; c1ccc(-c2ccc(N(c3ccc4oc5cc6ccc7ccccc7c6cc5c4c3)c3cccc4ccccc34)cc2)cc1]

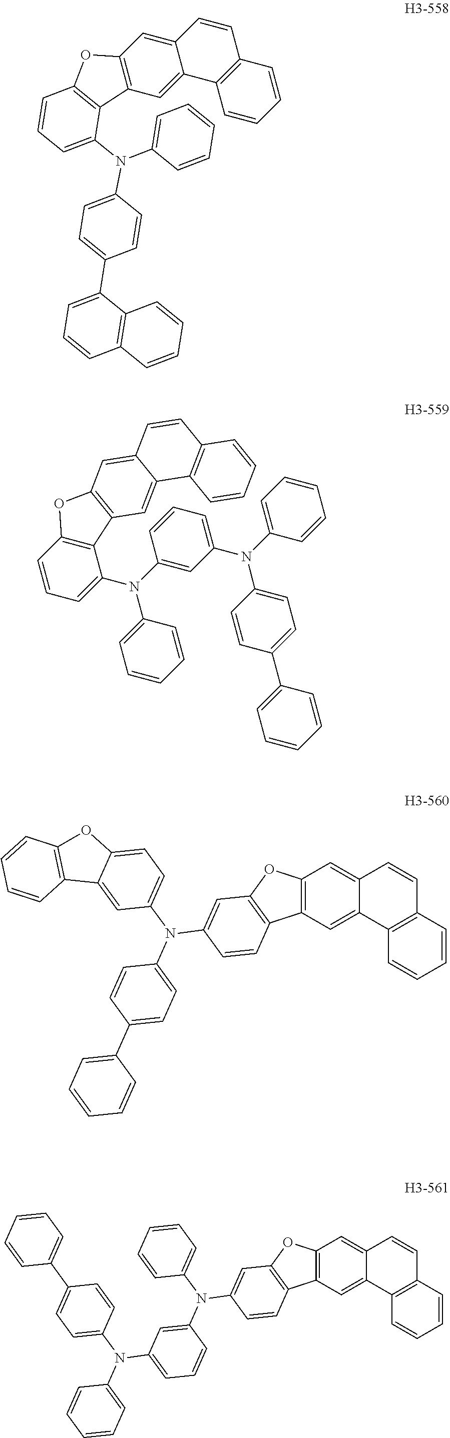 [SMILES: c1ccc(N(c2ccc(-c3cccc4ccccc34)cc2)c2cccc3oc4cc5ccc6ccccc6c5cc4c23)cc1 ; c1ccc(-c2ccc(N(c3ccccc3)c3cccc(N(c4ccccc4)c4cccc5oc6cc7ccc8ccccc8c7cc6c45)c3)cc2)cc1 ; c1ccc(-c2ccc(N(c3ccc4c(c3)oc3cc5ccc6ccccc6c5cc34)c3ccc4oc5ccccc5c4c3)cc2)cc1 ; c1ccc(-c2ccc(N(c3ccccc3)c3cccc(N(c4ccccc4)c4ccc5c(c4)oc4cc6ccc7ccccc7c6cc45)c3)cc2)cc1]

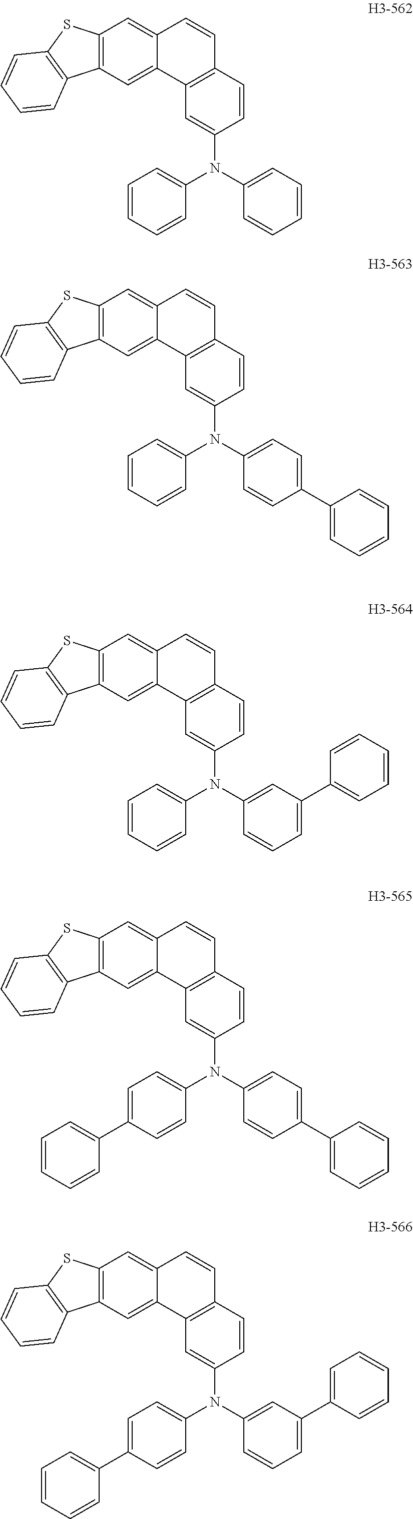 [SMILES: c1ccc(N(c2ccccc2)c2ccc3ccc4cc5sc6ccccc6c5cc4c3c2)cc1 ; c1ccc(-c2ccc(N(c3ccccc3)c3ccc4ccc5cc6sc7ccccc7c6cc5c4c3)cc2)cc1 ; c1ccc(-c2cccc(N(c3ccccc3)c3ccc4ccc5cc6sc7ccccc7c6cc5c4c3)c2)cc1 ; c1ccc(-c2ccc(N(c3ccc(-c4ccccc4)cc3)c3ccc4ccc5cc6sc7ccccc7c6cc5c4c3)cc2)cc1 ; c1ccc(-c2ccc(N(c3cccc(-c4ccccc4)c3)c3ccc4ccc5cc6sc7ccccc7c6cc5c4c3)cc2)cc1]

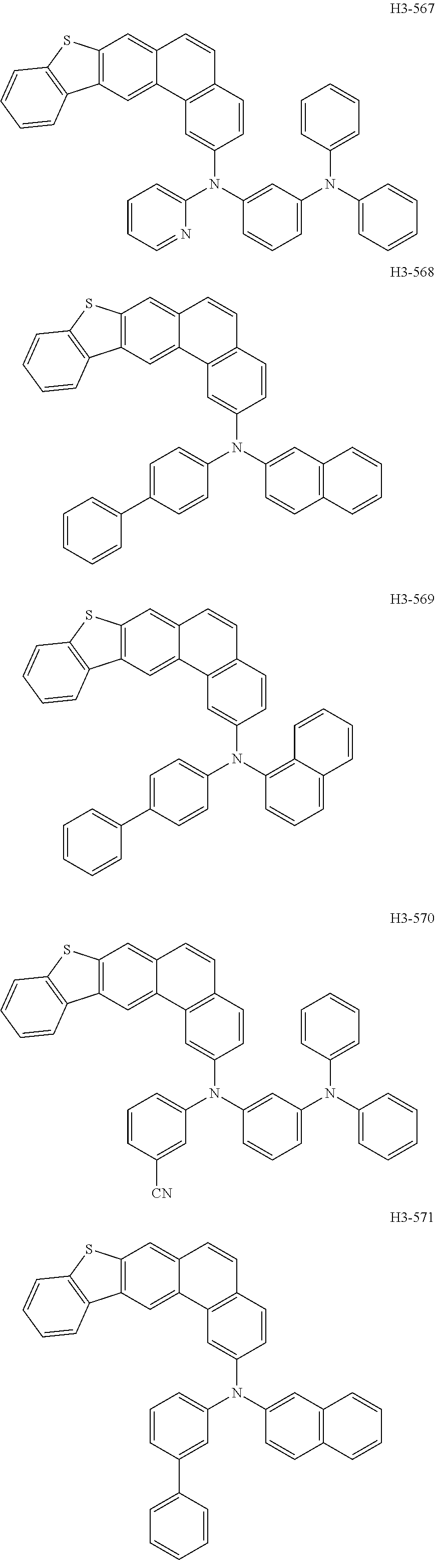 [SMILES: c1ccc(N(c2ccccc2)c2cccc(N(c3ccc4ccc5cc6sc7ccccc7c6cc5c4c3)c3ccccn3)c2)cc1 ; c1ccc(-c2ccc(N(c3ccc4ccccc4c3)c3ccc4ccc5cc6sc7ccccc7c6cc5c4c3)cc2)cc1 ; c1ccc(-c2ccc(N(c3ccc4ccc5cc6sc7ccccc7c6cc5c4c3)c3cccc4ccccc34)cc2)cc1 ; N#Cc1cccc(N(c2cccc(N(c3ccccc3)c3ccccc3)c2)c2ccc3ccc4cc5sc6ccccc6c5cc4c3c2)c1 ; c1ccc(-c2cccc(N(c3ccc4ccccc4c3)c3ccc4ccc5cc6sc7ccccc7c6cc5c4c3)c2)cc1]

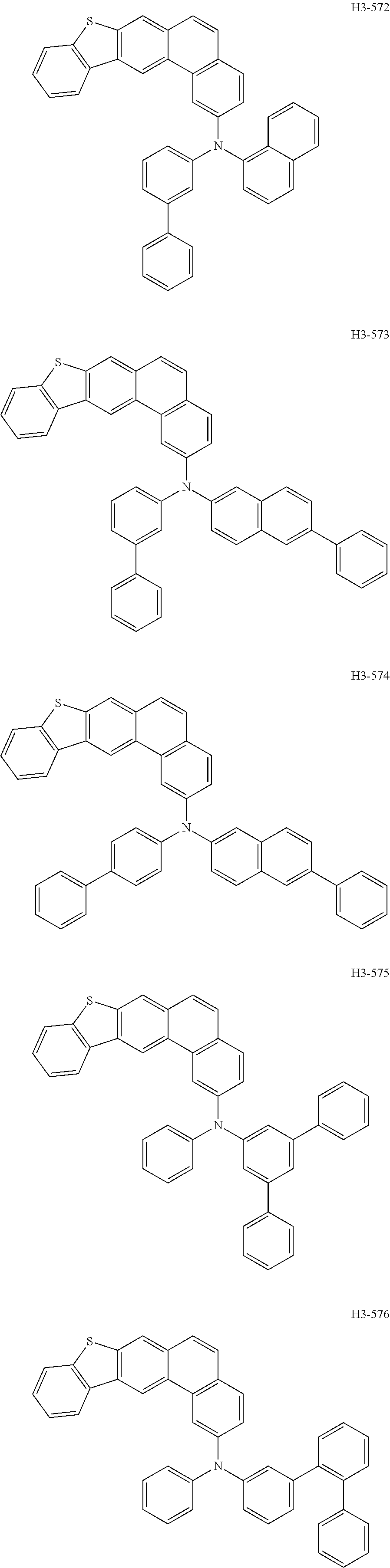 [SMILES: c1ccc(-c2cccc(N(c3ccc4ccc5cc6sc7ccccc7c6cc5c4c3)c3cccc4ccccc34)c2)cc1 ; c1ccc(-c2cccc(N(c3ccc4cc(-c5ccccc5)ccc4c3)c3ccc4ccc5cc6sc7ccccc7c6cc5c4c3)c2)cc1 ; c1ccc(-c2ccc(N(c3ccc4cc(-c5ccccc5)ccc4c3)c3ccc4ccc5cc6sc7ccccc7c6cc5c4c3)cc2)cc1 ; c1ccc(-c2cc(-c3ccccc3)cc(N(c3ccccc3)c3ccc4ccc5cc6sc7ccccc7c6cc5c4c3)c2)cc1 ; c1ccc(-c2ccccc2-c2cccc(N(c3ccccc3)c3ccc4ccc5cc6sc7ccccc7c6cc5c4c3)c2)cc1]

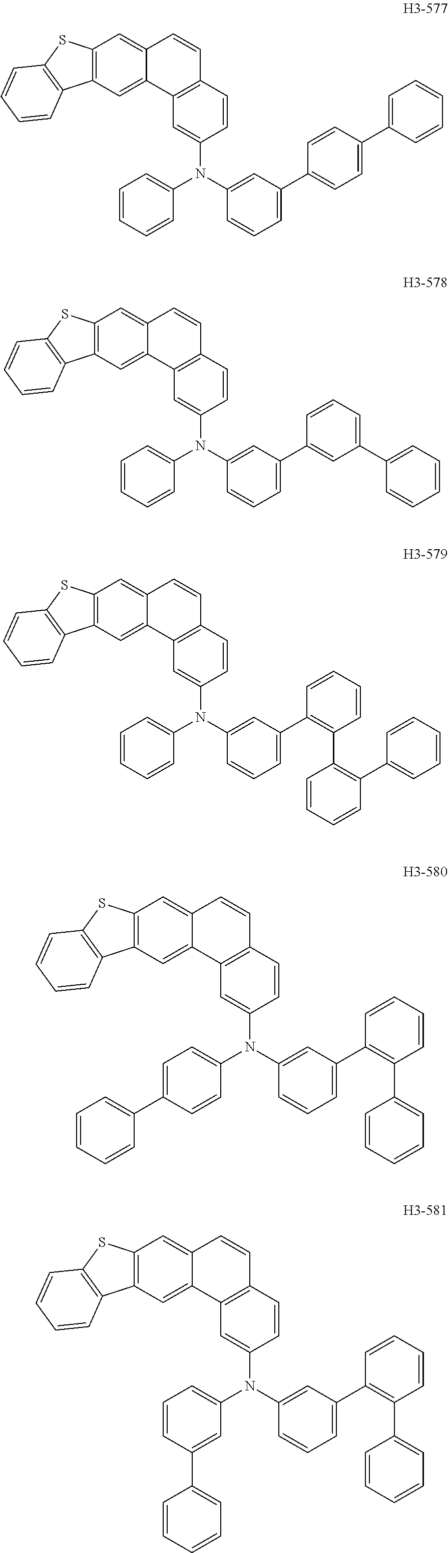 [SMILES: c1ccc(-c2ccc(-c3cccc(N(c4ccccc4)c4ccc5ccc6cc7sc8ccccc8c7cc6c5c4)c3)cc2)cc1 ; c1ccc(-c2cccc(-c3cccc(N(c4ccccc4)c4ccc5ccc6cc7sc8ccccc8c7cc6c5c4)c3)c2)cc1 ; c1ccc(-c2ccccc2-c2ccccc2-c2cccc(N(c3ccccc3)c3ccc4ccc5cc6sc7ccccc7c6cc5c4c3)c2)cc1 ; c1ccc(-c2ccc(N(c3cccc(-c4ccccc4-c4ccccc4)c3)c3ccc4ccc5cc6sc7ccccc7c6cc5c4c3)cc2)cc1 ; c1ccc(-c2cccc(N(c3cccc(-c4ccccc4-c4ccccc4)c3)c3ccc4ccc5cc6sc7ccccc7c6cc5c4c3)c2)cc1]

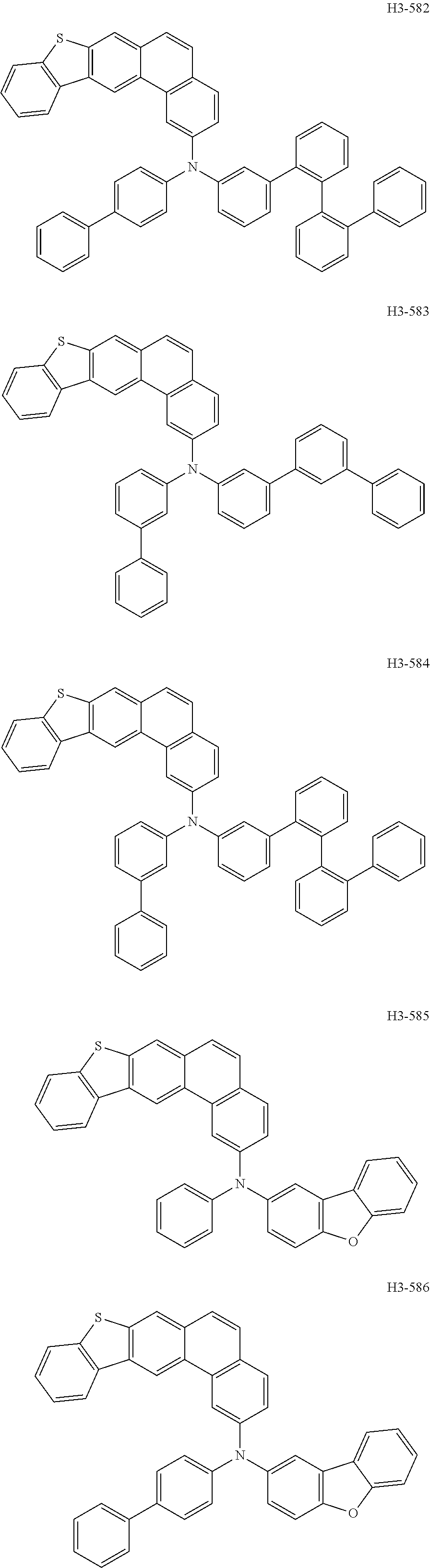 [SMILES: c1ccc(-c2ccc(N(c3cccc(-c4ccccc4-c4ccccc4-c4ccccc4)c3)c3ccc4ccc5cc6sc7ccccc7c6cc5c4c3)cc2)cc1 ; c1ccc(-c2cccc(-c3cccc(N(c4cccc(-c5ccccc5)c4)c4ccc5ccc6cc7sc8ccccc8c7cc6c5c4)c3)c2)cc1 ; c1ccc(-c2cccc(N(c3cccc(-c4ccccc4-c4ccccc4-c4ccccc4)c3)c3ccc4ccc5cc6sc7ccccc7c6cc5c4c3)c2)cc1 ; c1ccc(N(c2ccc3ccc4cc5sc6ccccc6c5cc4c3c2)c2ccc3oc4ccccc4c3c2)cc1 ; c1ccc(-c2ccc(N(c3ccc4ccc5cc6sc7ccccc7c6cc5c4c3)c3ccc4oc5ccccc5c4c3)cc2)cc1]

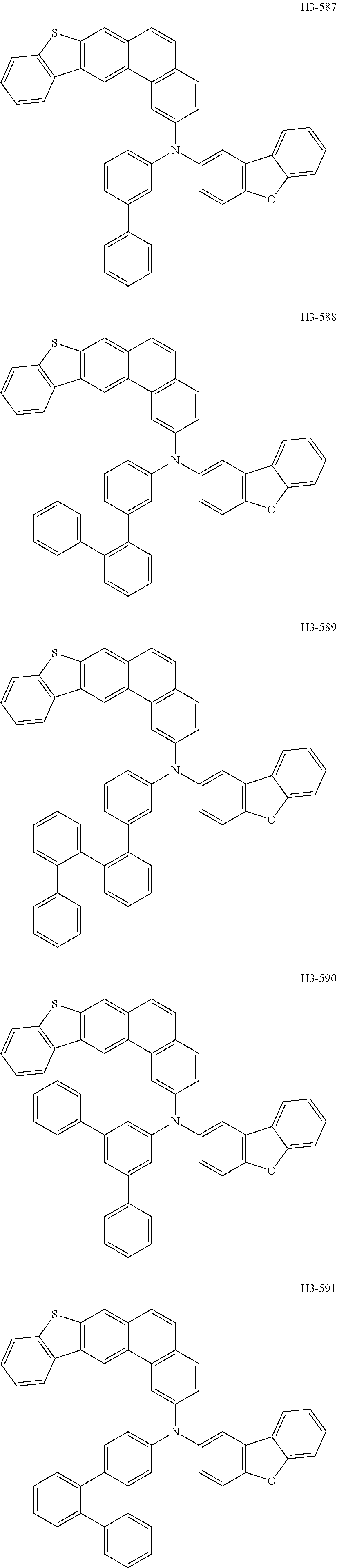 [SMILES: c1ccc(-c2cccc(N(c3ccc4ccc5cc6sc7ccccc7c6cc5c4c3)c3ccc4oc5ccccc5c4c3)c2)cc1 ; c1ccc(-c2ccccc2-c2cccc(N(c3ccc4ccc5cc6sc7ccccc7c6cc5c4c3)c3ccc4oc5ccccc5c4c3)c2)cc1 ; c1ccc(-c2ccccc2-c2ccccc2-c2cccc(N(c3ccc4ccc5cc6sc7ccccc7c6cc5c4c3)c3ccc4oc5ccccc5c4c3)c2)cc1 ; c1ccc(-c2cc(-c3ccccc3)cc(N(c3ccc4ccc5cc6sc7ccccc7c6cc5c4c3)c3ccc4oc5ccccc5c4c3)c2)cc1 ; c1ccc(-c2ccccc2-c2ccc(N(c3ccc4ccc5cc6sc7ccccc7c6cc5c4c3)c3ccc4oc5ccccc5c4c3)cc2)cc1]

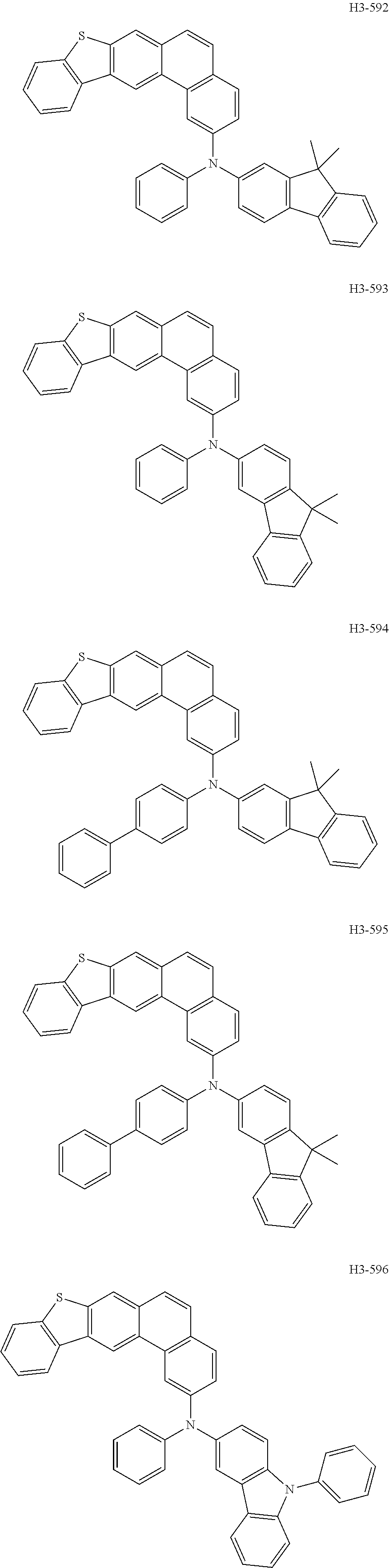 [SMILES: CC1(C)c2ccccc2-c2ccc(N(c3ccccc3)c3ccc4ccc5cc6sc7ccccc7c6cc5c4c3)cc21 ; CC1(C)c2ccccc2-c2cc(N(c3ccccc3)c3ccc4ccc5cc6sc7ccccc7c6cc5c4c3)ccc21 ; CC1(C)c2ccccc2-c2ccc(N(c3ccc(-c4ccccc4)cc3)c3ccc4ccc5cc6sc7ccccc7c6cc5c4c3)cc21 ; CC1(C)c2ccccc2-c2cc(N(c3ccc(-c4ccccc4)cc3)c3ccc4ccc5cc6sc7ccccc7c6cc5c4c3)ccc21 ; c1ccc(N(c2ccc3ccc4cc5sc6ccccc6c5cc4c3c2)c2ccc3c(c2)c2ccccc2n3-c2ccccc2)cc1]

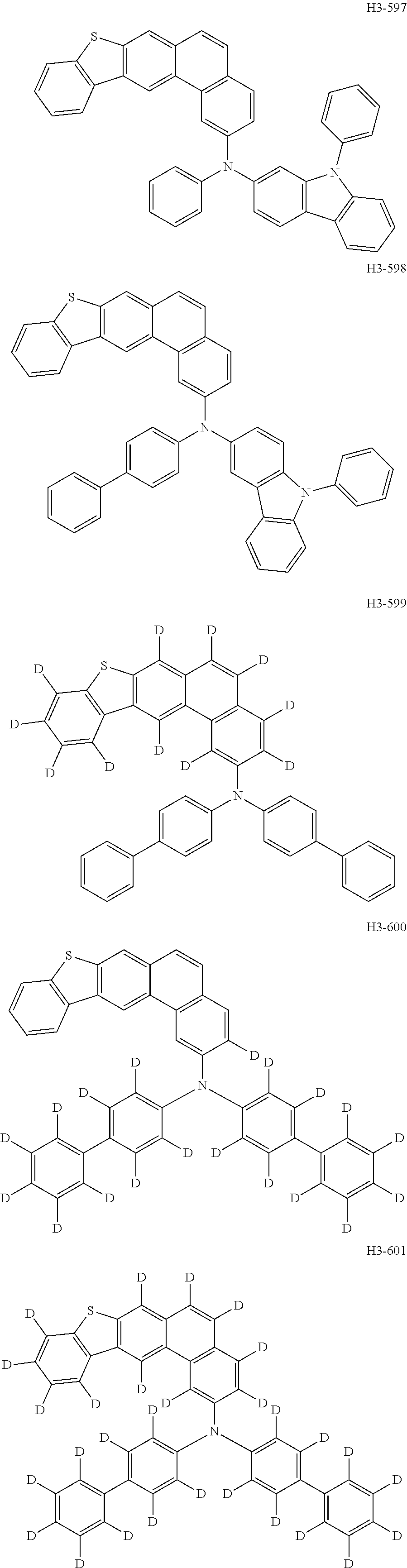

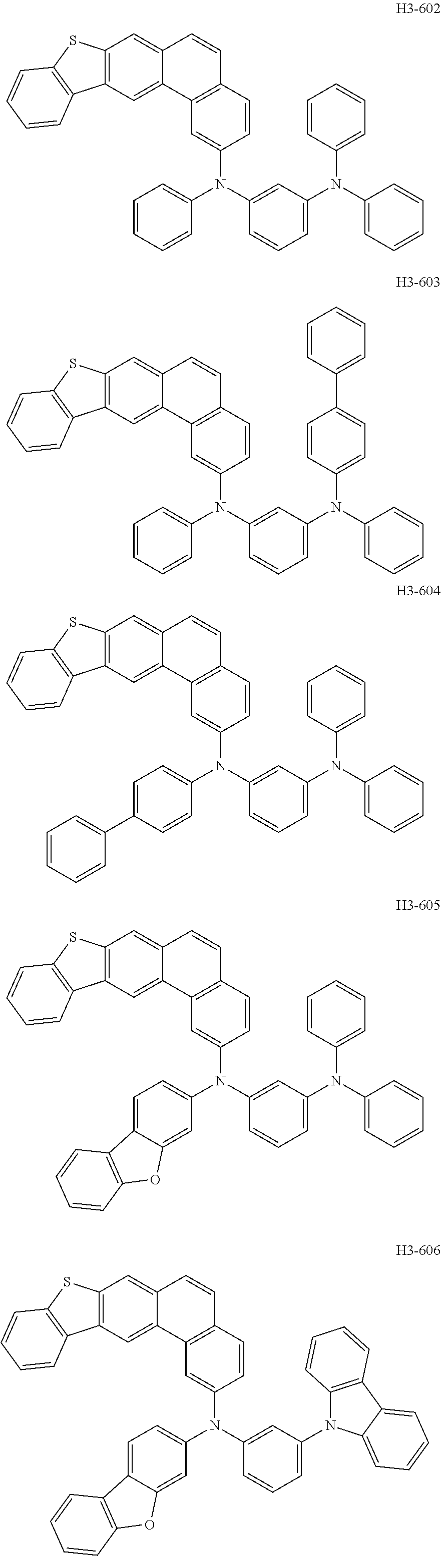 [SMILES: c1ccc(N(c2ccccc2)c2cccc(N(c3ccccc3)c3ccc4ccc5cc6sc7ccccc7c6cc5c4c3)c2)cc1 ; c1ccc(-c2ccc(N(c3ccccc3)c3cccc(N(c4ccccc4)c4ccc5ccc6cc7sc8ccccc8c7cc6c5c4)c3)cc2)cc1 ; c1ccc(-c2ccc(N(c3cccc(N(c4ccccc4)c4ccccc4)c3)c3ccc4ccc5cc6sc7ccccc7c6cc5c4c3)cc2)cc1 ; c1ccc(N(c2ccccc2)c2cccc(N(c3ccc4c(c3)oc3ccccc34)c3ccc4ccc5cc6sc7ccccc7c6cc5c4c3)c2)cc1 ; c1cc(N(c2ccc3c(c2)oc2ccccc23)c2ccc3ccc4cc5sc6ccccc6c5cc4c3c2)cc(-n2c3ccccc3c3ccccc32)c1]

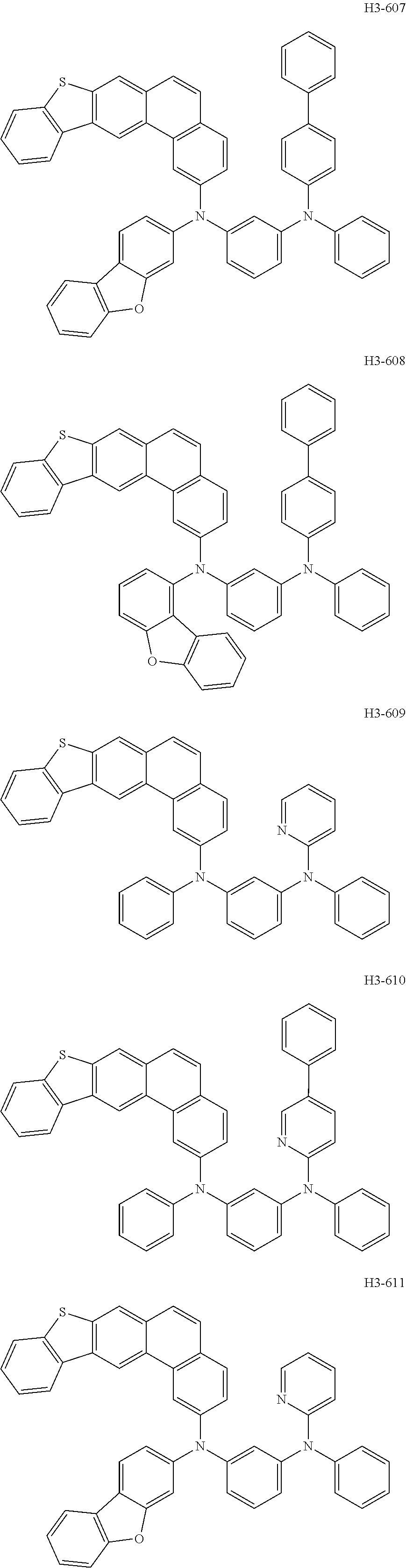 [SMILES: c1ccc(-c2ccc(N(c3ccccc3)c3cccc(N(c4ccc5c(c4)oc4ccccc45)c4ccc5ccc6cc7sc8ccccc8c7cc6c5c4)c3)cc2)cc1 ; c1ccc(-c2ccc(N(c3ccccc3)c3cccc(N(c4ccc5ccc6cc7sc8ccccc8c7cc6c5c4)c4cccc5oc6ccccc6c45)c3)cc2)cc1 ; c1ccc(N(c2cccc(N(c3ccccc3)c3ccccn3)c2)c2ccc3ccc4cc5sc6ccccc6c5cc4c3c2)cc1 ; c1ccc(-c2ccc(N(c3ccccc3)c3cccc(N(c4ccccc4)c4ccc5ccc6cc7sc8ccccc8c7cc6c5c4)c3)nc2)cc1 ; c1ccc(N(c2cccc(N(c3ccc4c(c3)oc3ccccc34)c3ccc4ccc5cc6sc7ccccc7c6cc5c4c3)c2)c2ccccn2)cc1]

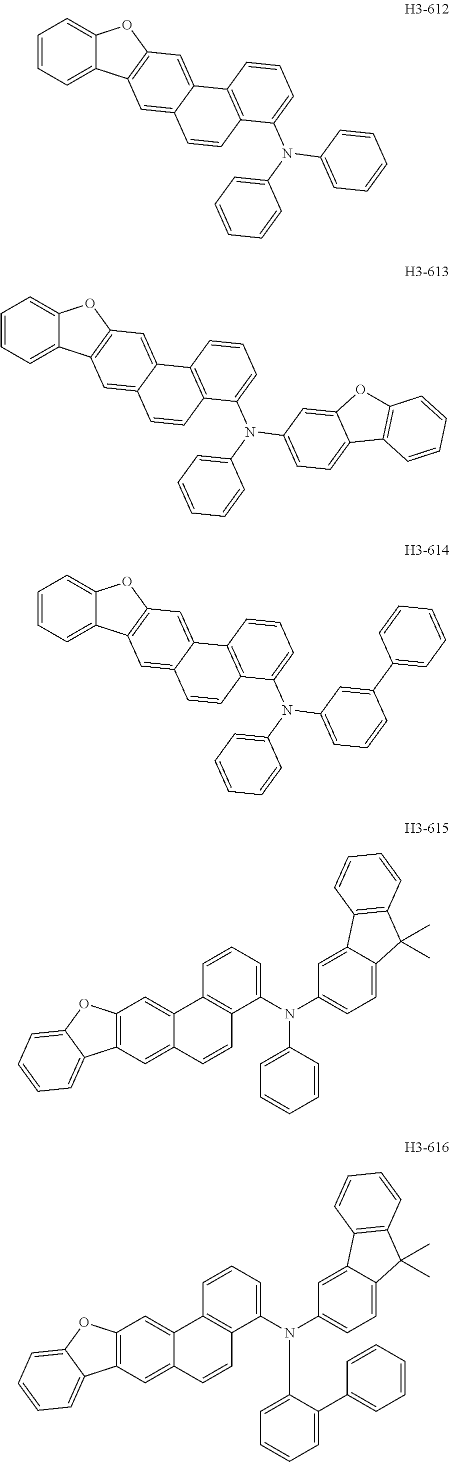 [SMILES: c1ccc(N(c2ccccc2)c2cccc3c2ccc2cc4c(cc23)oc2ccccc24)cc1 ; c1ccc(N(c2ccc3c(c2)oc2ccccc23)c2cccc3c2ccc2cc4c(cc23)oc2ccccc24)cc1 ; c1ccc(-c2cccc(N(c3ccccc3)c3cccc4c3ccc3cc5c(cc34)oc3ccccc35)c2)cc1 ; CC1(C)c2ccccc2-c2cc(N(c3ccccc3)c3cccc4c3ccc3cc5c(cc34)oc3ccccc35)ccc21 ; CC1(C)c2ccccc2-c2cc(N(c3ccccc3-c3ccccc3)c3cccc4c3ccc3cc5c(cc34)oc3ccccc35)ccc21]

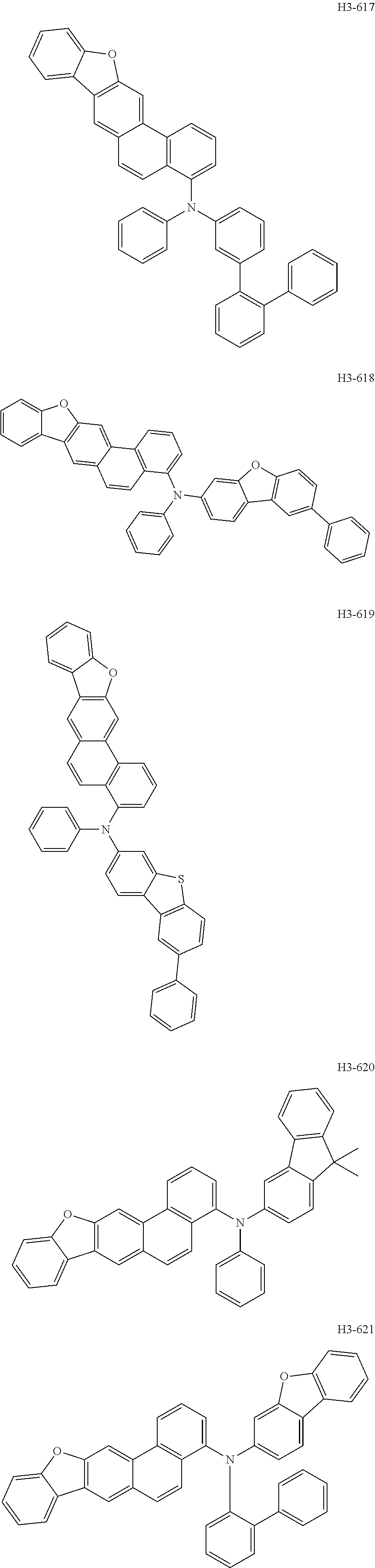 [SMILES: c1ccc(-c2ccccc2-c2cccc(N(c3ccccc3)c3cccc4c3ccc3cc5c(cc34)oc3ccccc35)c2)cc1 ; c1ccc(-c2ccc3oc4cc(N(c5ccccc5)c5cccc6c5ccc5cc7c(cc56)oc5ccccc57)ccc4c3c2)cc1 ; c1ccc(-c2ccc3sc4cc(N(c5ccccc5)c5cccc6c5ccc5cc7c(cc56)oc5ccccc57)ccc4c3c2)cc1 ; CC1(C)c2ccccc2-c2cc(N(c3ccccc3)c3cccc4c3ccc3cc5c(cc34)oc3ccccc35)ccc21 ; c1ccc(-c2ccccc2N(c2ccc3c(c2)oc2ccccc23)c2cccc3c2ccc2cc4c(cc23)oc2ccccc24)cc1]

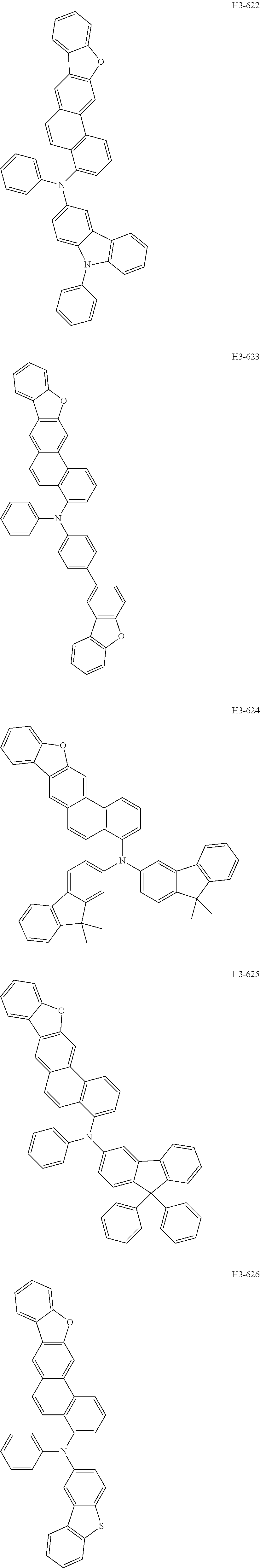 [SMILES: c1ccc(N(c2ccc3c(c2)c2ccccc2n3-c2ccccc2)c2cccc3c2ccc2cc4c(cc23)oc2ccccc24)cc1 ; c1ccc(N(c2ccc(-c3ccc4oc5ccccc5c4c3)cc2)c2cccc3c2ccc2cc4c(cc23)oc2ccccc24)cc1 ; CC1(C)c2ccccc2-c2cc(N(c3ccc4c(c3)C(C)(C)c3ccccc3-4)c3cccc4c3ccc3cc5c(cc34)oc3ccccc35)ccc21 ; c1ccc(N(c2ccc3c(c2)-c2ccccc2C3(c2ccccc2)c2ccccc2)c2cccc3c2ccc2cc4c(cc23)oc2ccccc24)cc1 ; c1ccc(N(c2ccc3sc4ccccc4c3c2)c2cccc3c2ccc2cc4c(cc23)oc2ccccc24)cc1]

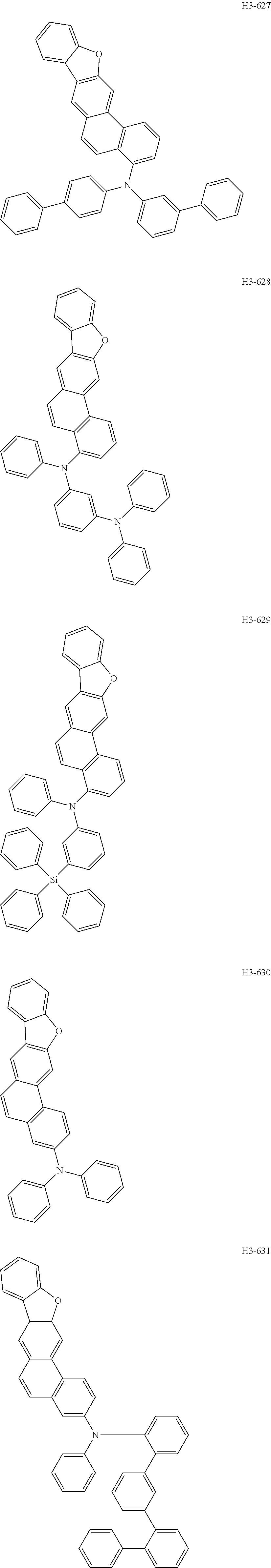 [SMILES: c1ccc(-c2ccc(N(c3cccc(-c4ccccc4)c3)c3cccc4c3ccc3cc5c(cc34)oc3ccccc35)cc2)cc1 ; c1ccc(N(c2ccccc2)c2cccc(N(c3ccccc3)c3cccc4c3ccc3cc5c(cc34)oc3ccccc35)c2)cc1 ; c1ccc(N(c2cccc([Si](c3ccccc3)(c3ccccc3)c3ccccc3)c2)c2cccc3c2ccc2cc4c(cc23)oc2ccccc24)cc1 ; c1ccc(N(c2ccccc2)c2ccc3c(ccc4cc5c(cc43)oc3ccccc35)c2)cc1 ; c1ccc(-c2ccccc2-c2cccc(-c3ccccc3N(c3ccccc3)c3ccc4c(ccc5cc6c(cc54)oc4ccccc46)c3)c2)cc1]

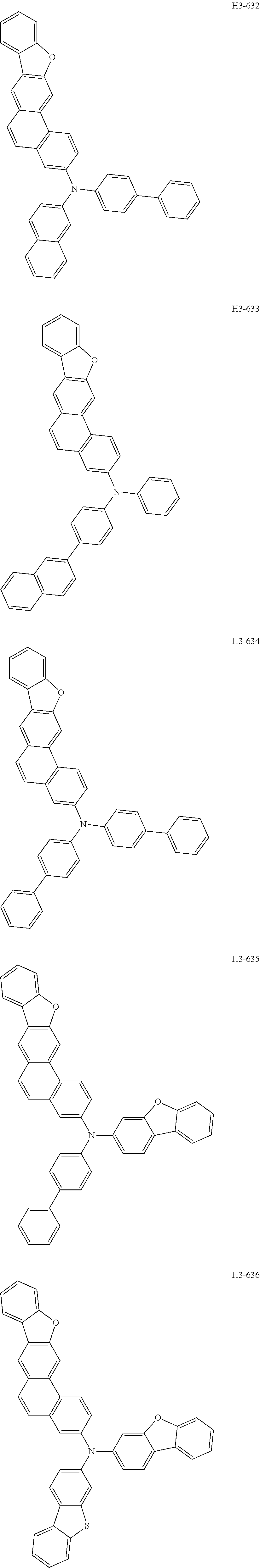 [SMILES: c1ccc(-c2ccc(N(c3ccc4ccccc4c3)c3ccc4c(ccc5cc6c(cc54)oc4ccccc46)c3)cc2)cc1 ; c1ccc(N(c2ccc(-c3ccc4ccccc4c3)cc2)c2ccc3c(ccc4cc5c(cc43)oc3ccccc35)c2)cc1 ; c1ccc(-c2ccc(N(c3ccc(-c4ccccc4)cc3)c3ccc4c(ccc5cc6c(cc54)oc4ccccc46)c3)cc2)cc1 ; c1ccc(-c2ccc(N(c3ccc4c(ccc5cc6c(cc54)oc4ccccc46)c3)c3ccc4c(c3)oc3ccccc34)cc2)cc1 ; c1ccc2c(c1)oc1cc(N(c3ccc4c(ccc5cc6c(cc54)oc4ccccc46)c3)c3ccc4c(c3)sc3ccccc34)ccc12]

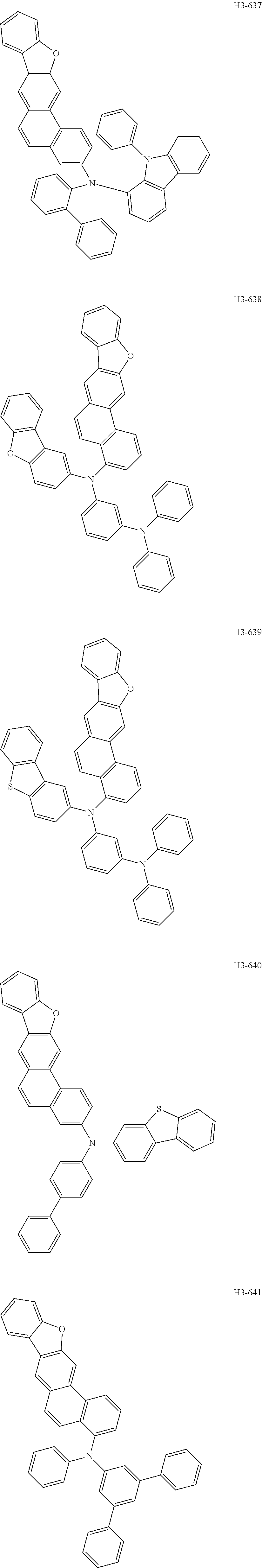 [SMILES: c1ccc(-c2ccccc2N(c2ccc3c(ccc4cc5c(cc43)oc3ccccc35)c2)c2cccc3c4ccccc4n(-c4ccccc4)c23)cc1 ; c1ccc(N(c2ccccc2)c2cccc(N(c3ccc4oc5ccccc5c4c3)c3cccc4c3ccc3cc5c(cc34)oc3ccccc35)c2)cc1 ; c1ccc(N(c2ccccc2)c2cccc(N(c3ccc4sc5ccccc5c4c3)c3cccc4c3ccc3cc5c(cc34)oc3ccccc35)c2)cc1 ; c1ccc(-c2ccc(N(c3ccc4c(ccc5cc6c(cc54)oc4ccccc46)c3)c3ccc4c(c3)sc3ccccc34)cc2)cc1 ; c1ccc(-c2cc(-c3ccccc3)cc(N(c3ccccc3)c3cccc4c3ccc3cc5c(cc34)oc3ccccc35)c2)cc1]

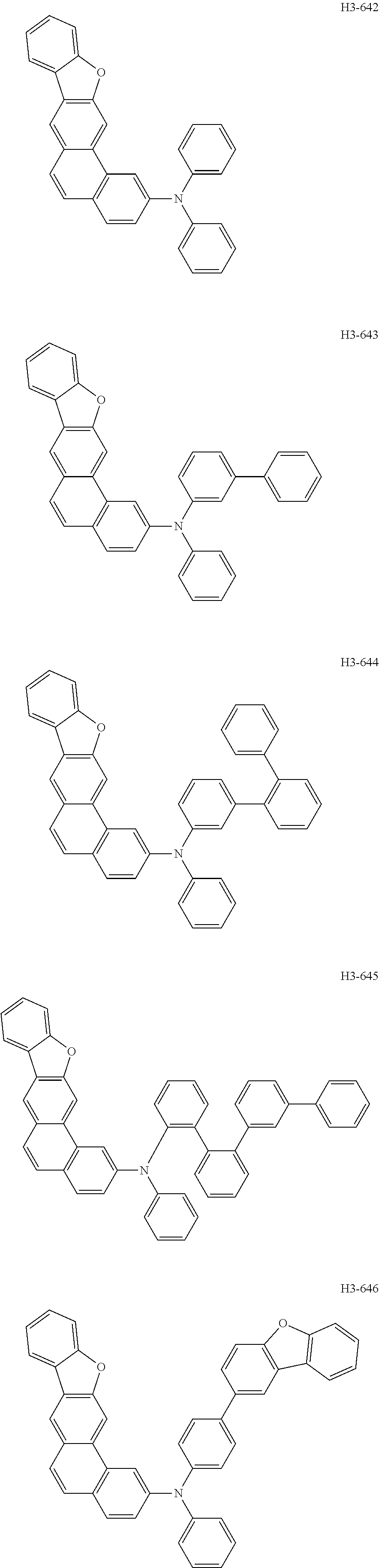 [SMILES: c1ccc(N(c2ccccc2)c2ccc3ccc4cc5c(cc4c3c2)oc2ccccc25)cc1 ; c1ccc(-c2cccc(N(c3ccccc3)c3ccc4ccc5cc6c(cc5c4c3)oc3ccccc36)c2)cc1 ; c1ccc(-c2ccccc2-c2cccc(N(c3ccccc3)c3ccc4ccc5cc6c(cc5c4c3)oc3ccccc36)c2)cc1 ; c1ccc(-c2cccc(-c3ccccc3-c3ccccc3N(c3ccccc3)c3ccc4ccc5cc6c(cc5c4c3)oc3ccccc36)c2)cc1 ; c1ccc(N(c2ccc(-c3ccc4oc5ccccc5c4c3)cc2)c2ccc3ccc4cc5c(cc4c3c2)oc2ccccc25)cc1]

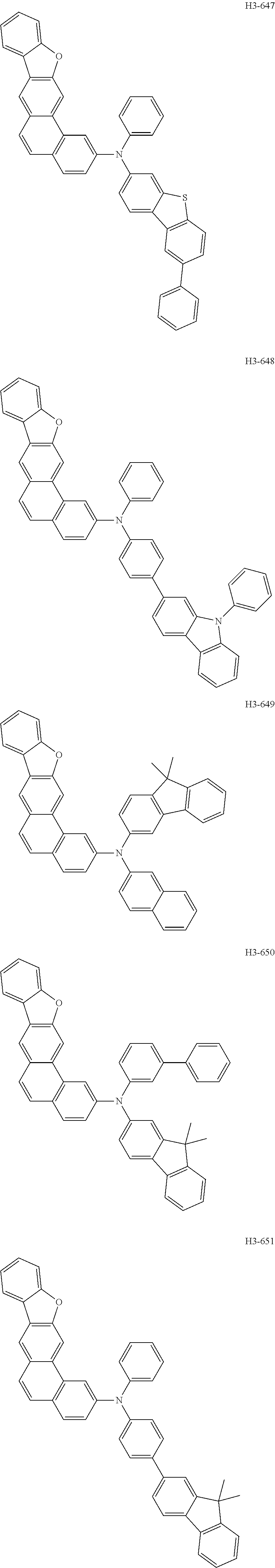 [SMILES: c1ccc(-c2ccc3sc4cc(N(c5ccccc5)c5ccc6ccc7cc8c(cc7c6c5)oc5ccccc58)ccc4c3c2)cc1 ; c1ccc(N(c2ccc(-c3ccc4c5ccccc5n(-c5ccccc5)c4c3)cc2)c2ccc3ccc4cc5c(cc4c3c2)oc2ccccc25)cc1 ; CC1(C)c2ccccc2-c2cc(N(c3ccc4ccccc4c3)c3ccc4ccc5cc6c(cc5c4c3)oc3ccccc36)ccc21 ; CC1(C)c2ccccc2-c2ccc(N(c3cccc(-c4ccccc4)c3)c3ccc4ccc5cc6c(cc5c4c3)oc3ccccc36)cc21 ; CC1(C)c2ccccc2-c2ccc(-c3ccc(N(c4ccccc4)c4ccc5ccc6cc7c(cc6c5c4)oc4ccccc47)cc3)cc21]

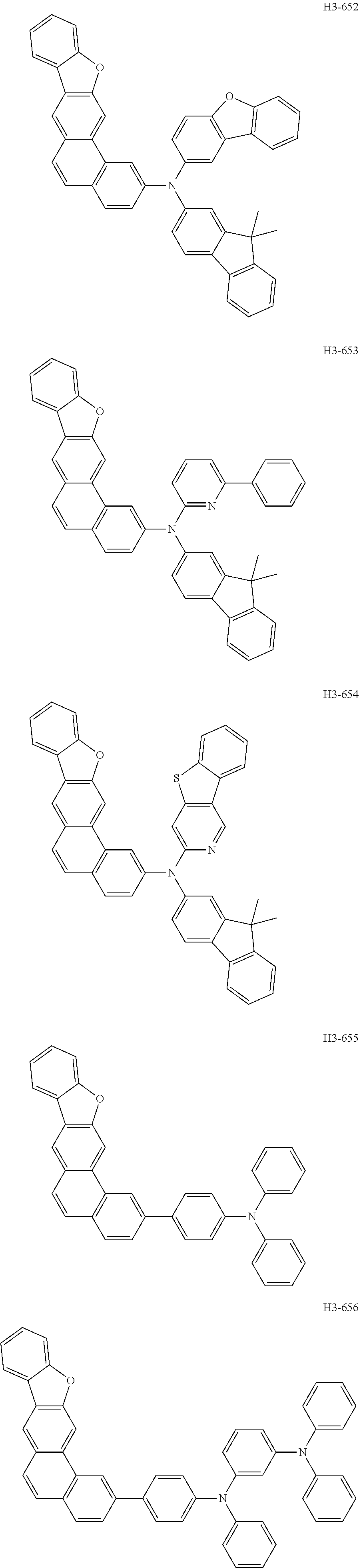 [SMILES: CC1(C)c2ccccc2-c2ccc(N(c3ccc4ccc5cc6c(cc5c4c3)oc3ccccc36)c3ccc4oc5ccccc5c4c3)cc21 ; CC1(C)c2ccccc2-c2ccc(N(c3ccc4ccc5cc6c(cc5c4c3)oc3ccccc36)c3cccc(-c4ccccc4)n3)cc21 ; CC1(C)c2ccccc2-c2ccc(N(c3ccc4ccc5cc6c(cc5c4c3)oc3ccccc36)c3cc4sc5ccccc5c4cn3)cc21 ; c1ccc(N(c2ccccc2)c2ccc(-c3ccc4ccc5cc6c(cc5c4c3)oc3ccccc36)cc2)cc1 ; c1ccc(N(c2ccccc2)c2cccc(N(c3ccccc3)c3ccc(-c4ccc5ccc6cc7c(cc6c5c4)oc4ccccc47)cc3)c2)cc1]

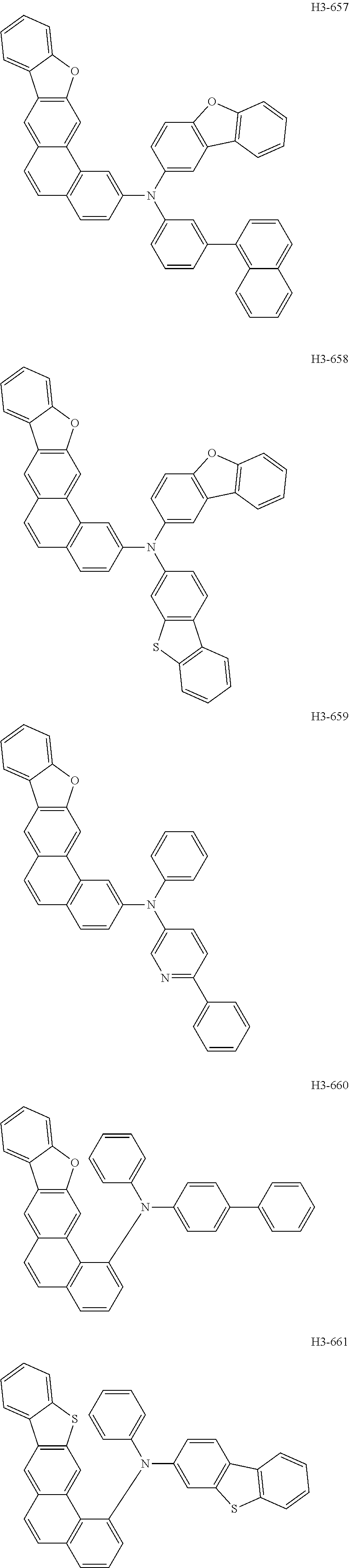 [SMILES: c1cc(-c2cccc3ccccc23)cc(N(c2ccc3ccc4cc5c(cc4c3c2)oc2ccccc25)c2ccc3oc4ccccc4c3c2)c1 ; c1ccc2c(c1)oc1ccc(N(c3ccc4c(c3)sc3ccccc34)c3ccc4ccc5cc6c(cc5c4c3)oc3ccccc36)cc12 ; c1ccc(-c2ccc(N(c3ccccc3)c3ccc4ccc5cc6c(cc5c4c3)oc3ccccc36)cn2)cc1 ; c1ccc(-c2ccc(N(c3ccccc3)c3cccc4ccc5cc6c(cc5c34)oc3ccccc36)cc2)cc1 ; c1ccc(N(c2ccc3c(c2)sc2ccccc23)c2cccc3ccc4cc5c(cc4c23)sc2ccccc25)cc1]

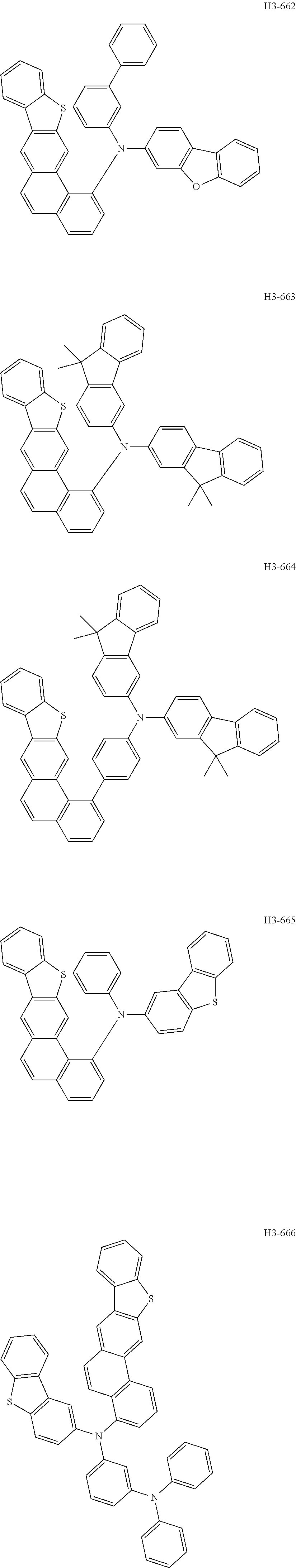 [SMILES: c1ccc(-c2cccc(N(c3ccc4c(c3)oc3ccccc34)c3cccc4ccc5cc6c(cc5c34)sc3ccccc36)c2)cc1 ; CC1(C)c2ccccc2-c2cc(N(c3ccc4c(c3)C(C)(C)c3ccccc3-4)c3cccc4ccc5cc6c(cc5c34)sc3ccccc36)ccc21 ; CC1(C)c2ccccc2-c2cc(N(c3ccc(-c4cccc5ccc6cc7c(cc6c45)sc4ccccc47)cc3)c3ccc4c(c3)C(C)(C)c3ccccc3-4)ccc21 ; c1ccc(N(c2ccc3sc4ccccc4c3c2)c2cccc3ccc4cc5c(cc4c23)sc2ccccc25)cc1 ; c1ccc(N(c2ccccc2)c2cccc(N(c3ccc4sc5ccccc5c4c3)c3cccc4c3ccc3cc5c(cc34)sc3ccccc35)c2)cc1]

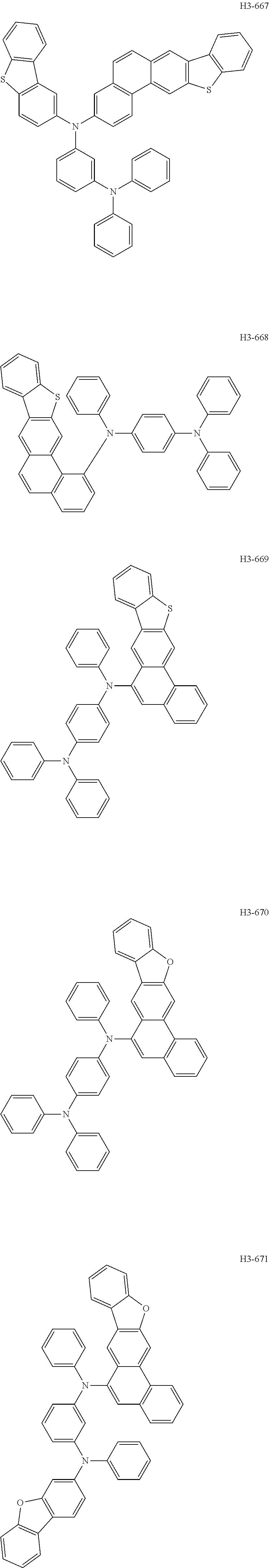 [SMILES: c1ccc(N(c2ccccc2)c2cccc(N(c3ccc4c(ccc5cc6c(cc54)sc4ccccc46)c3)c3ccc4sc5ccccc5c4c3)c2)cc1 ; c1ccc(N(c2ccccc2)c2ccc(N(c3ccccc3)c3cccc4ccc5cc6c(cc5c34)sc3ccccc36)cc2)cc1 ; c1ccc(N(c2ccccc2)c2ccc(N(c3ccccc3)c3cc4ccccc4c4cc5sc6ccccc6c5cc34)cc2)cc1 ; c1ccc(N(c2ccccc2)c2ccc(N(c3ccccc3)c3cc4ccccc4c4cc5oc6ccccc6c5cc34)cc2)cc1 ; c1ccc(N(c2cccc(N(c3ccccc3)c3cc4ccccc4c4cc5oc6ccccc6c5cc34)c2)c2ccc3c(c2)oc2ccccc23)cc1]

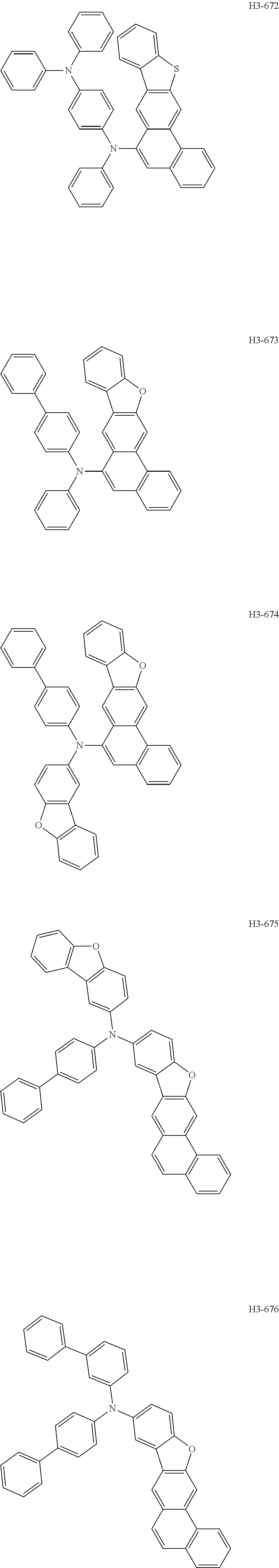 [SMILES: c1ccc(N(c2ccccc2)c2ccc(N(c3ccccc3)c3cc4ccccc4c4cc5sc6ccccc6c5cc34)cc2)cc1 ; c1ccc(-c2ccc(N(c3ccccc3)c3cc4ccccc4c4cc5oc6ccccc6c5cc34)cc2)cc1 ; c1ccc(-c2ccc(N(c3ccc4oc5ccccc5c4c3)c3cc4ccccc4c4cc5oc6ccccc6c5cc34)cc2)cc1 ; c1ccc(-c2ccc(N(c3ccc4oc5ccccc5c4c3)c3ccc4oc5cc6c(ccc7ccccc76)cc5c4c3)cc2)cc1 ; c1ccc(-c2ccc(N(c3cccc(-c4ccccc4)c3)c3ccc4oc5cc6c(ccc7ccccc76)cc5c4c3)cc2)cc1]

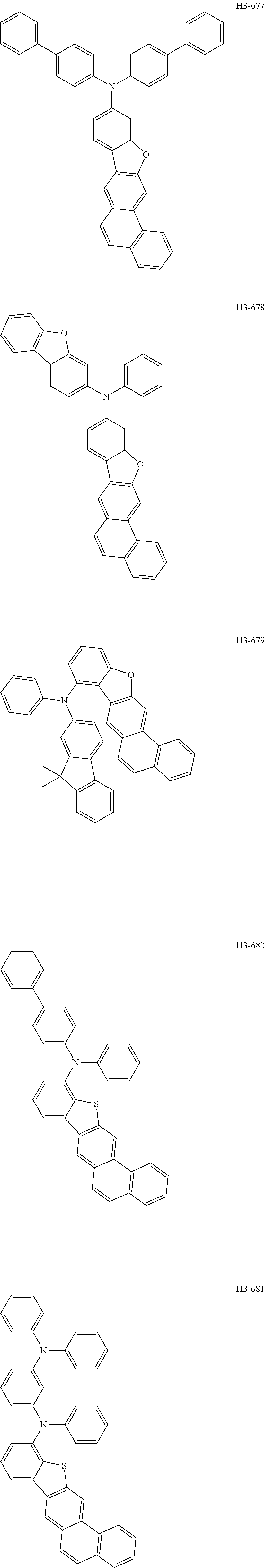 [SMILES: c1ccc(-c2ccc(N(c3ccc(-c4ccccc4)cc3)c3ccc4c(c3)oc3cc5c(ccc6ccccc65)cc34)cc2)cc1 ; c1ccc(N(c2ccc3c(c2)oc2ccccc23)c2ccc3c(c2)oc2cc4c(ccc5ccccc54)cc23)cc1 ; CC1(C)c2ccccc2-c2ccc(N(c3ccccc3)c3cccc4oc5cc6c(ccc7ccccc76)cc5c34)cc21 ; c1ccc(-c2ccc(N(c3ccccc3)c3cccc4c3sc3cc5c(ccc6ccccc65)cc34)cc2)cc1 ; c1ccc(N(c2ccccc2)c2cccc(N(c3ccccc3)c3cccc4c3sc3cc5c(ccc6ccccc65)cc34)c2)cc1]

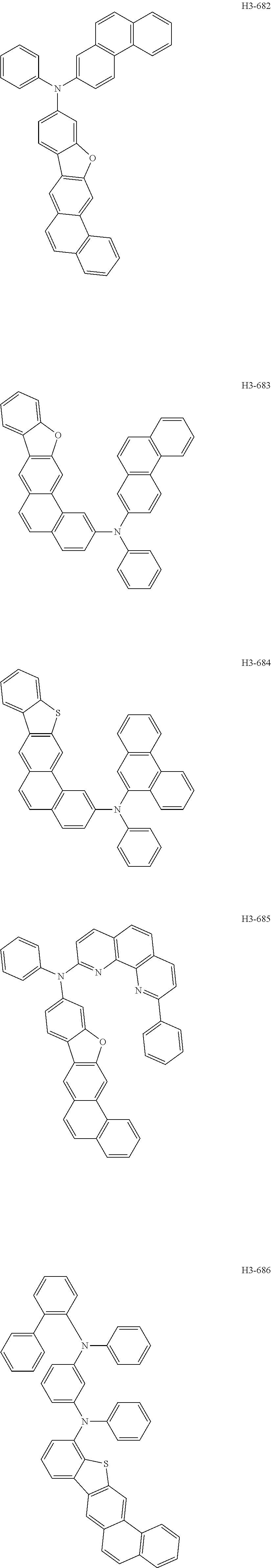 [SMILES: c1ccc(N(c2ccc3c(ccc4ccccc43)c2)c2ccc3c(c2)oc2cc4c(ccc5ccccc54)cc23)cc1 ; c1ccc(N(c2ccc3c(ccc4ccccc43)c2)c2ccc3ccc4cc5c(cc4c3c2)oc2ccccc25)cc1 ; c1ccc(N(c2ccc3ccc4cc5c(cc4c3c2)sc2ccccc25)c2cc3ccccc3c3ccccc23)cc1 ; c1ccc(-c2ccc3ccc4ccc(N(c5ccccc5)c5ccc6c(c5)oc5cc7c(ccc8ccccc87)cc56)nc4c3n2)cc1 ; c1ccc(-c2ccccc2N(c2ccccc2)c2cccc(N(c3ccccc3)c3cccc4c3sc3cc5c(ccc6ccccc65)cc34)c2)cc1]

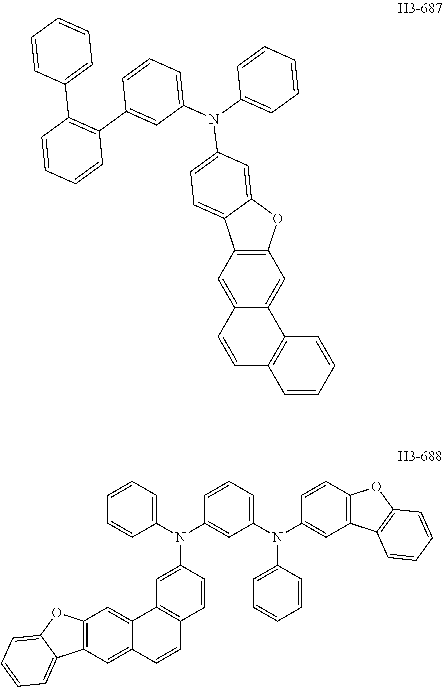 [SMILES: c1ccc(-c2ccccc2-c2cccc(N(c3ccccc3)c3ccc4c(c3)oc3cc5c(ccc6ccccc65)cc34)c2)cc1 ; c1ccc(N(c2cccc(N(c3ccccc3)c3ccc4oc5ccccc5c4c3)c2)c2ccc3ccc4cc5c(cc4c3c2)oc2ccccc25)cc1]

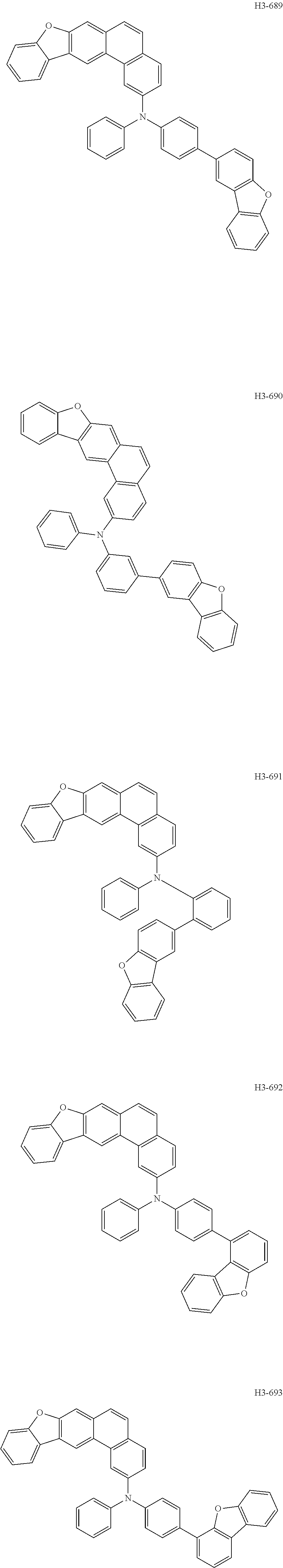 [SMILES: c1ccc(N(c2ccc(-c3ccc4oc5ccccc5c4c3)cc2)c2ccc3ccc4cc5oc6ccccc6c5cc4c3c2)cc1 ; c1ccc(N(c2cccc(-c3ccc4oc5ccccc5c4c3)c2)c2ccc3ccc4cc5oc6ccccc6c5cc4c3c2)cc1 ; c1ccc(N(c2ccc3ccc4cc5oc6ccccc6c5cc4c3c2)c2ccccc2-c2ccc3oc4ccccc4c3c2)cc1 ; c1ccc(N(c2ccc(-c3cccc4oc5ccccc5c34)cc2)c2ccc3ccc4cc5oc6ccccc6c5cc4c3c2)cc1 ; c1ccc(N(c2ccc(-c3cccc4c3oc3ccccc34)cc2)c2ccc3ccc4cc5oc6ccccc6c5cc4c3c2)cc1]

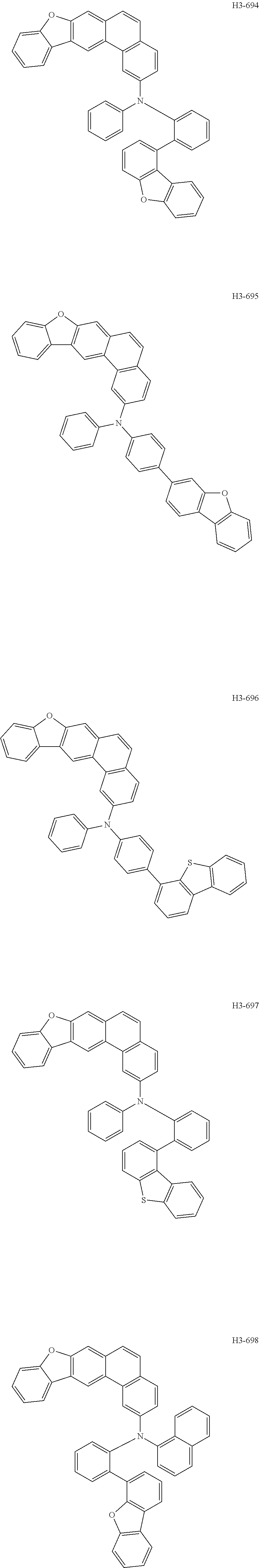 [SMILES: c1ccc(N(c2ccc3ccc4cc5oc6ccccc6c5cc4c3c2)c2ccccc2-c2cccc3oc4ccccc4c23)cc1 ; c1ccc(N(c2ccc(-c3ccc4c(c3)oc3ccccc34)cc2)c2ccc3ccc4cc5oc6ccccc6c5cc4c3c2)cc1 ; c1ccc(N(c2ccc(-c3cccc4c3sc3ccccc34)cc2)c2ccc3ccc4cc5oc6ccccc6c5cc4c3c2)cc1 ; c1ccc(N(c2ccc3ccc4cc5oc6ccccc6c5cc4c3c2)c2ccccc2-c2cccc3sc4ccccc4c23)cc1 ; c1ccc(N(c2ccc3ccc4cc5oc6ccccc6c5cc4c3c2)c2cccc3ccccc23)c(-c2cccc3c2oc2ccccc23)c1]

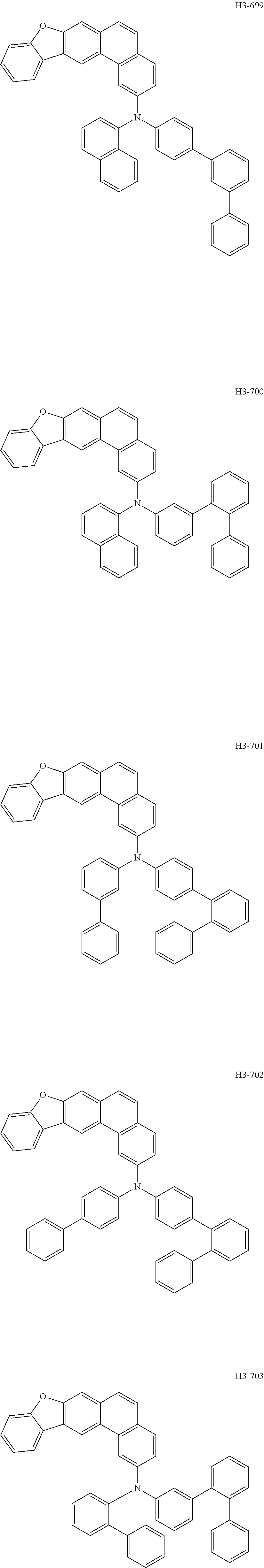 [SMILES: c1ccc(-c2cccc(-c3ccc(N(c4ccc5ccc6cc7oc8ccccc8c7cc6c5c4)c4cccc5ccccc45)cc3)c2)cc1 ; c1ccc(-c2ccccc2-c2cccc(N(c3ccc4ccc5cc6oc7ccccc7c6cc5c4c3)c3cccc4ccccc34)c2)cc1 ; c1ccc(-c2cccc(N(c3ccc(-c4ccccc4-c4ccccc4)cc3)c3ccc4ccc5cc6oc7ccccc7c6cc5c4c3)c2)cc1 ; c1ccc(-c2ccc(N(c3ccc(-c4ccccc4-c4ccccc4)cc3)c3ccc4ccc5cc6oc7ccccc7c6cc5c4c3)cc2)cc1 ; c1ccc(-c2ccccc2-c2cccc(N(c3ccc4ccc5cc6oc7ccccc7c6cc5c4c3)c3ccccc3-c3ccccc3)c2)cc1]

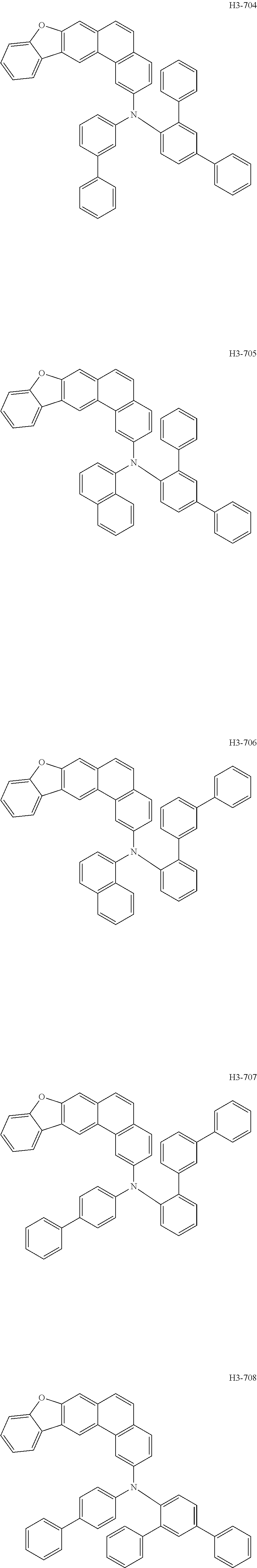 [SMILES: c1ccc(-c2cccc(N(c3ccc4ccc5cc6oc7ccccc7c6cc5c4c3)c3ccc(-c4ccccc4)cc3-c3ccccc3)c2)cc1 ; c1ccc(-c2ccc(N(c3ccc4ccc5cc6oc7ccccc7c6cc5c4c3)c3cccc4ccccc34)c(-c3ccccc3)c2)cc1 ; c1ccc(-c2cccc(-c3ccccc3N(c3ccc4ccc5cc6oc7ccccc7c6cc5c4c3)c3cccc4ccccc34)c2)cc1 ; c1ccc(-c2ccc(N(c3ccc4ccc5cc6oc7ccccc7c6cc5c4c3)c3ccccc3-c3cccc(-c4ccccc4)c3)cc2)cc1 ; c1ccc(-c2ccc(N(c3ccc4ccc5cc6oc7ccccc7c6cc5c4c3)c3ccc(-c4ccccc4)cc3-c3ccccc3)cc2)cc1]

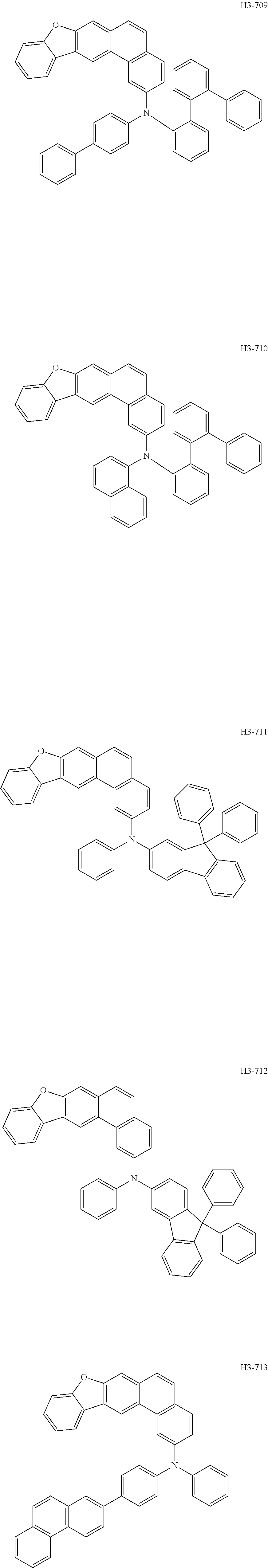 [SMILES: c1ccc(-c2ccc(N(c3ccc4ccc5cc6oc7ccccc7c6cc5c4c3)c3ccccc3-c3ccccc3-c3ccccc3)cc2)cc1 ; c1ccc(-c2ccccc2-c2ccccc2N(c2ccc3ccc4cc5oc6ccccc6c5cc4c3c2)c2cccc3ccccc23)cc1 ; c1ccc(N(c2ccc3c(c2)C(c2ccccc2)(c2ccccc2)c2ccccc2-3)c2ccc3ccc4cc5oc6ccccc6c5cc4c3c2)cc1 ; c1ccc(N(c2ccc3c(c2)-c2ccccc2C3(c2ccccc2)c2ccccc2)c2ccc3ccc4cc5oc6ccccc6c5cc4c3c2)cc1 ; c1ccc(N(c2ccc(-c3ccc4c(ccc5ccccc54)c3)cc2)c2ccc3ccc4cc5oc6ccccc6c5cc4c3c2)cc1]

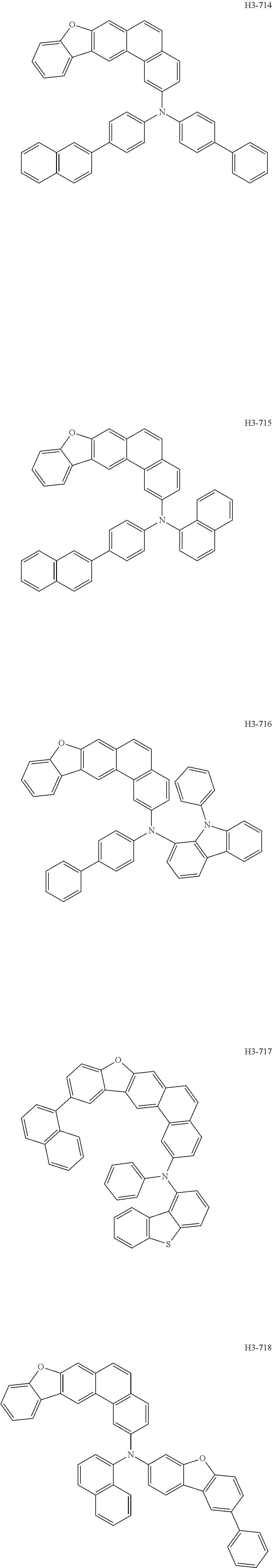 [SMILES: c1ccc(-c2ccc(N(c3ccc(-c4ccc5ccccc5c4)cc3)c3ccc4ccc5cc6oc7ccccc7c6cc5c4c3)cc2)cc1 ; c1ccc2cc(-c3ccc(N(c4ccc5ccc6cc7oc8ccccc8c7cc6c5c4)c4cccc5ccccc45)cc3)ccc2c1 ; c1ccc(-c2ccc(N(c3ccc4ccc5cc6oc7ccccc7c6cc5c4c3)c3cccc4c5ccccc5n(-c5ccccc5)c34)cc2)cc1 ; c1ccc(N(c2ccc3ccc4cc5oc6ccc(-c7cccc8ccccc78)cc6c5cc4c3c2)c2cccc3sc4ccccc4c23)cc1 ; c1ccc(-c2ccc3oc4cc(N(c5ccc6ccc7cc8oc9ccccc9c8cc7c6c5)c5cccc6ccccc56)ccc4c3c2)cc1]

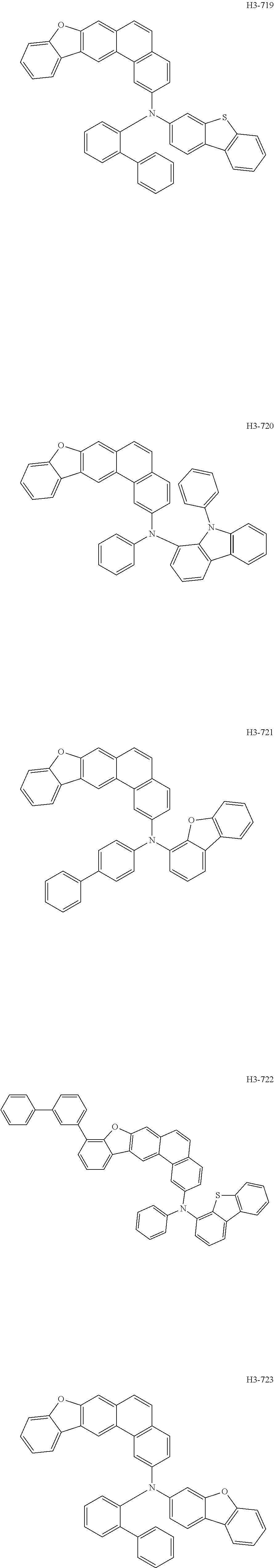 [SMILES: c1ccc(-c2ccccc2N(c2ccc3c(c2)sc2ccccc23)c2ccc3ccc4cc5oc6ccccc6c5cc4c3c2)cc1 ; c1ccc(N(c2ccc3ccc4cc5oc6ccccc6c5cc4c3c2)c2cccc3c4ccccc4n(-c4ccccc4)c23)cc1 ; c1ccc(-c2ccc(N(c3ccc4ccc5cc6oc7ccccc7c6cc5c4c3)c3cccc4c3oc3ccccc34)cc2)cc1 ; c1ccc(-c2cccc(-c3cccc4c3oc3cc5ccc6ccc(N(c7ccccc7)c7cccc8c7sc7ccccc78)cc6c5cc34)c2)cc1 ; c1ccc(-c2ccccc2N(c2ccc3c(c2)oc2ccccc23)c2ccc3ccc4cc5oc6ccccc6c5cc4c3c2)cc1]

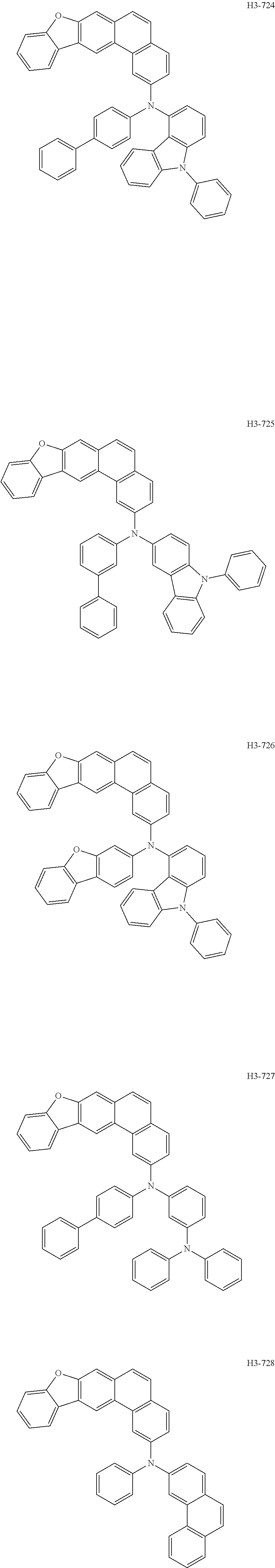 [SMILES: c1ccc(-c2ccc(N(c3ccc4ccc5cc6oc7ccccc7c6cc5c4c3)c3cccc4c3c3ccccc3n4-c3ccccc3)cc2)cc1 ; c1ccc(-c2cccc(N(c3ccc4ccc5cc6oc7ccccc7c6cc5c4c3)c3ccc4c(c3)c3ccccc3n4-c3ccccc3)c2)cc1 ; c1ccc(-n2c3ccccc3c3c(N(c4ccc5c(c4)oc4ccccc45)c4ccc5ccc6cc7oc8ccccc8c7cc6c5c4)cccc32)cc1 ; c1ccc(-c2ccc(N(c3cccc(N(c4ccccc4)c4ccccc4)c3)c3ccc4ccc5cc6oc7ccccc7c6cc5c4c3)cc2)cc1 ; c1ccc(N(c2ccc3ccc4ccccc4c3c2)c2ccc3ccc4cc5oc6ccccc6c5cc4c3c2)cc1]

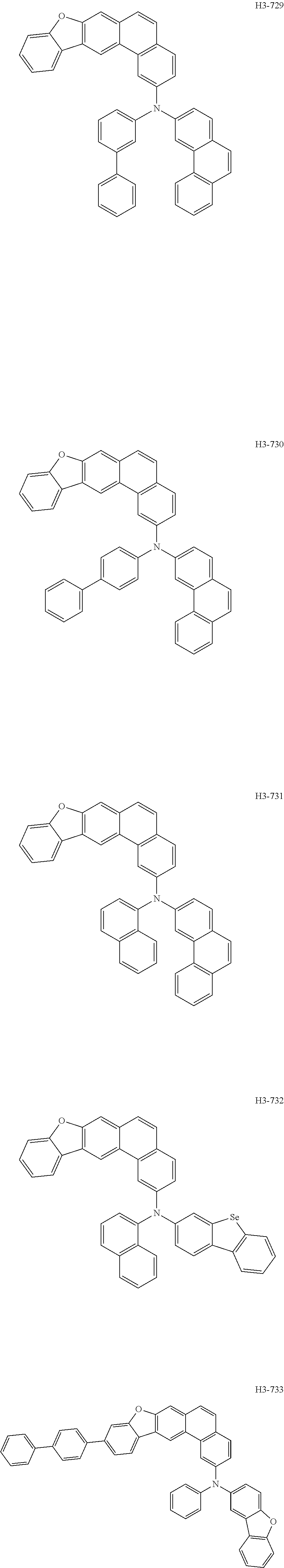 [SMILES: c1ccc(-c2cccc(N(c3ccc4ccc5ccccc5c4c3)c3ccc4ccc5cc6oc7ccccc7c6cc5c4c3)c2)cc1 ; c1ccc(-c2ccc(N(c3ccc4ccc5ccccc5c4c3)c3ccc4ccc5cc6oc7ccccc7c6cc5c4c3)cc2)cc1 ; c1ccc2c(N(c3ccc4ccc5ccccc5c4c3)c3ccc4ccc5cc6oc7ccccc7c6cc5c4c3)cccc2c1 ; c1ccc2c(N(c3ccc4c(c3)[se]c3ccccc34)c3ccc4ccc5cc6oc7ccccc7c6cc5c4c3)cccc2c1 ; c1ccc(-c2ccc(-c3ccc4c(c3)oc3cc5ccc6ccc(N(c7ccccc7)c7ccc8oc9ccccc9c8c7)cc6c5cc34)cc2)cc1]

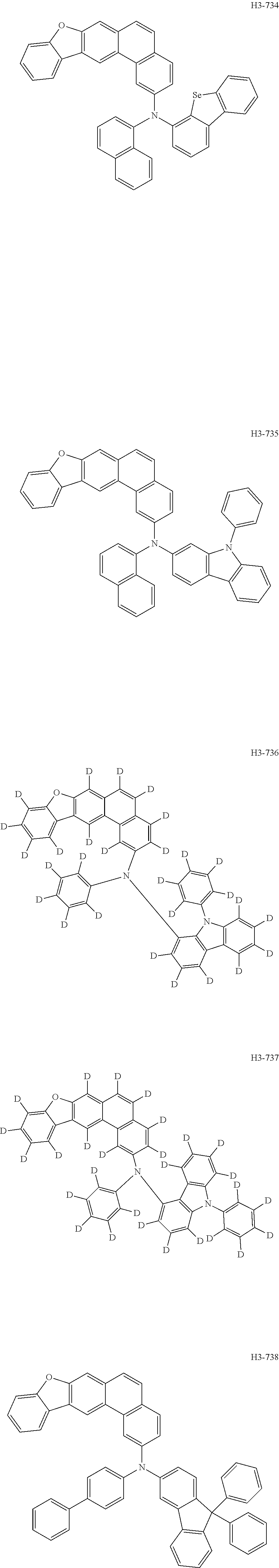

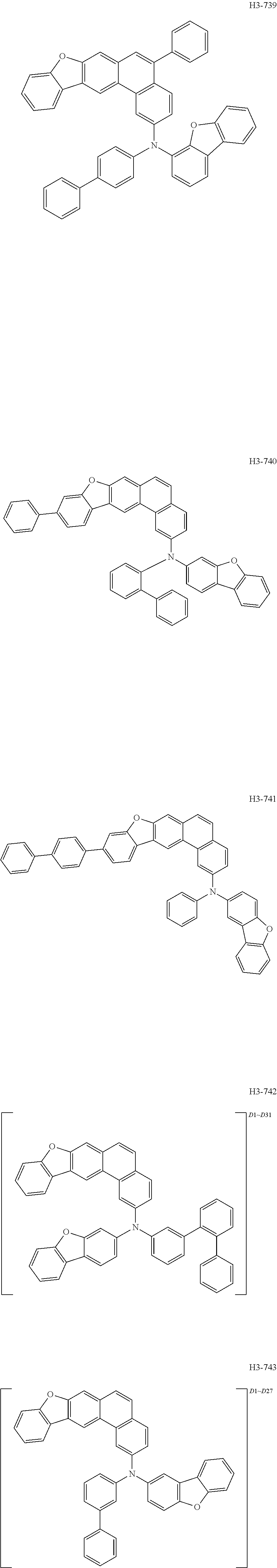

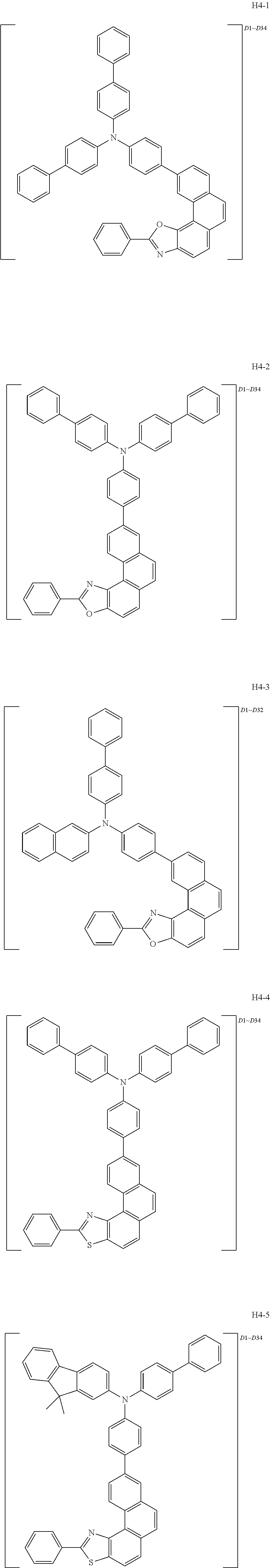

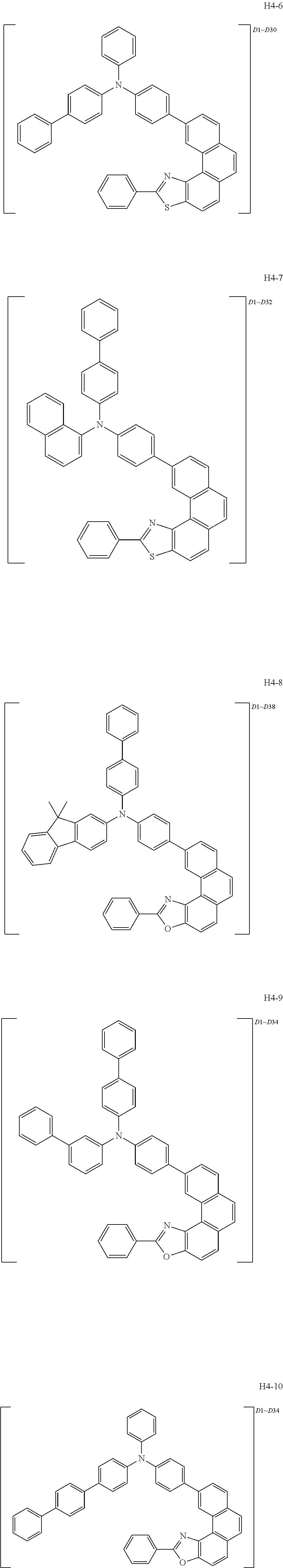

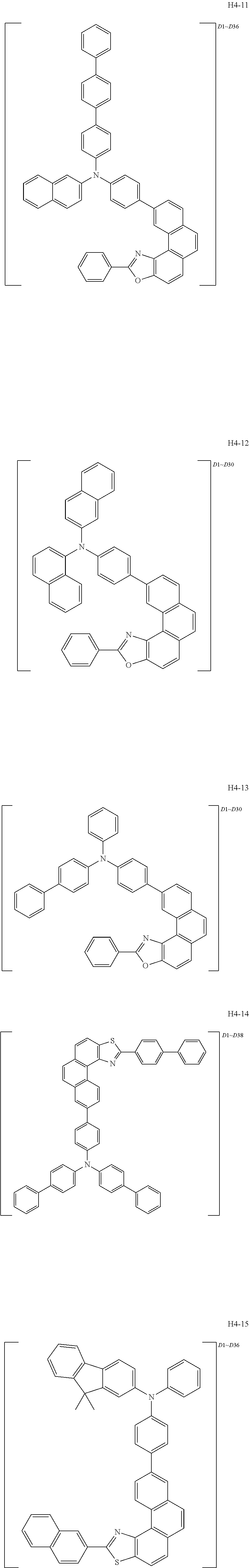

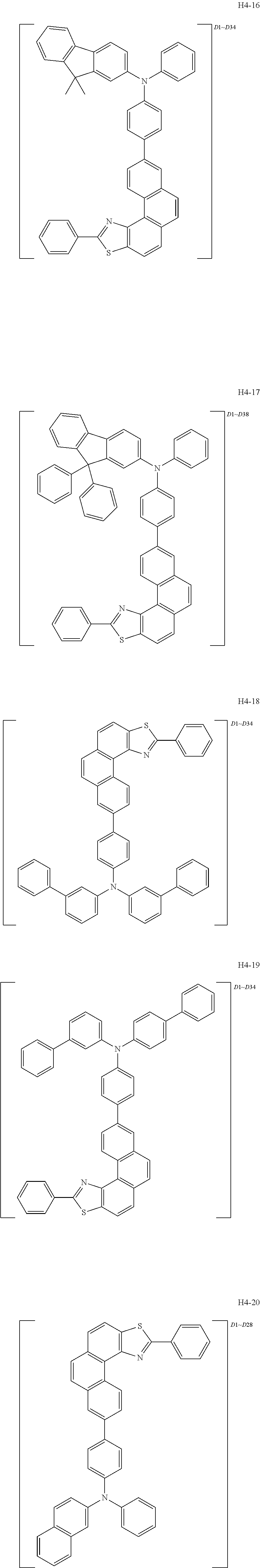

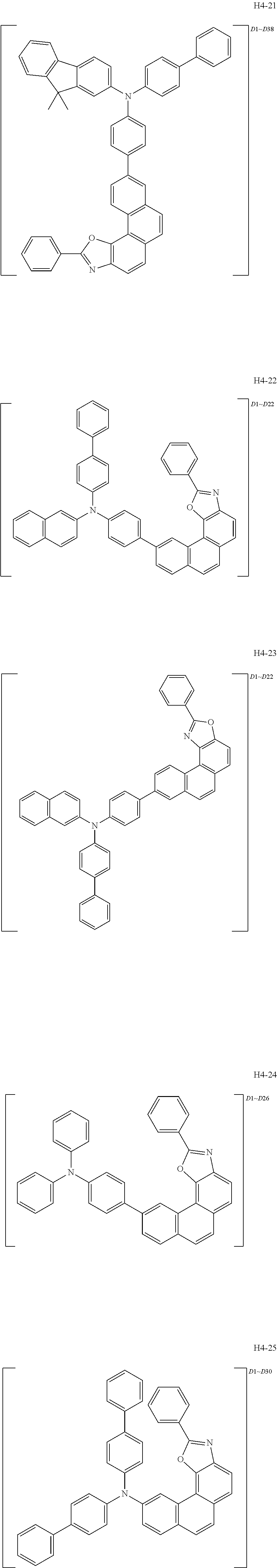

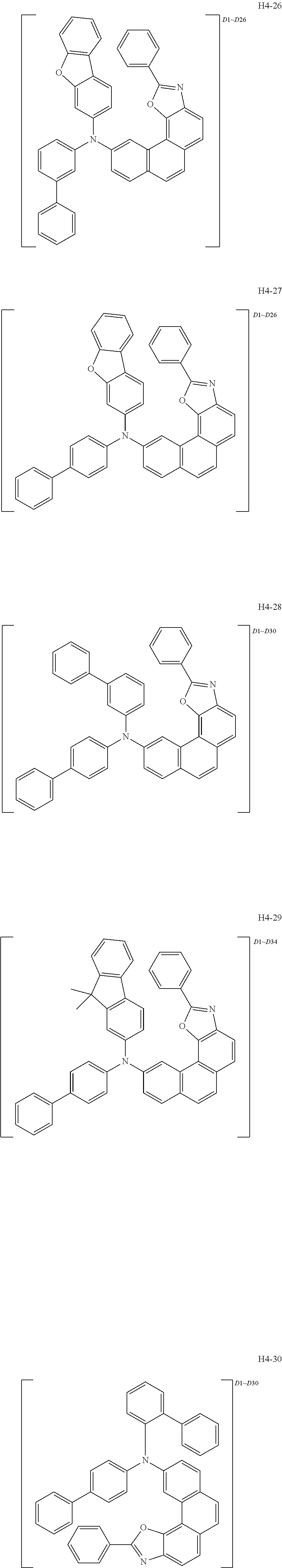

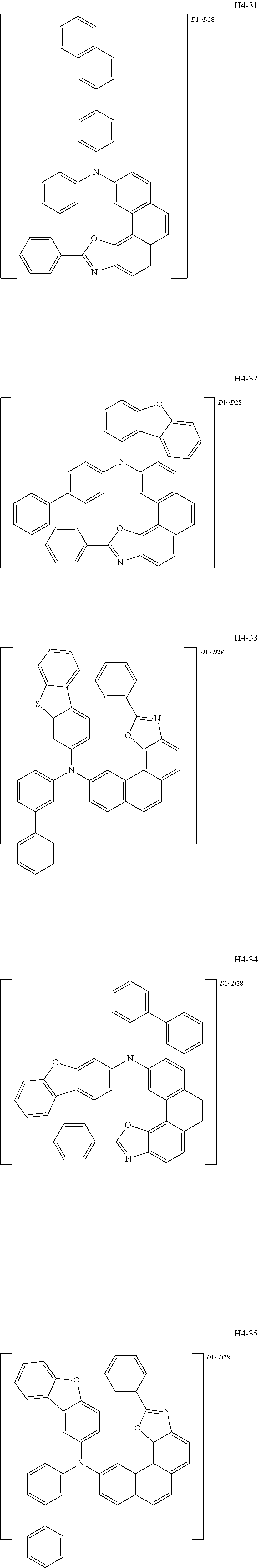

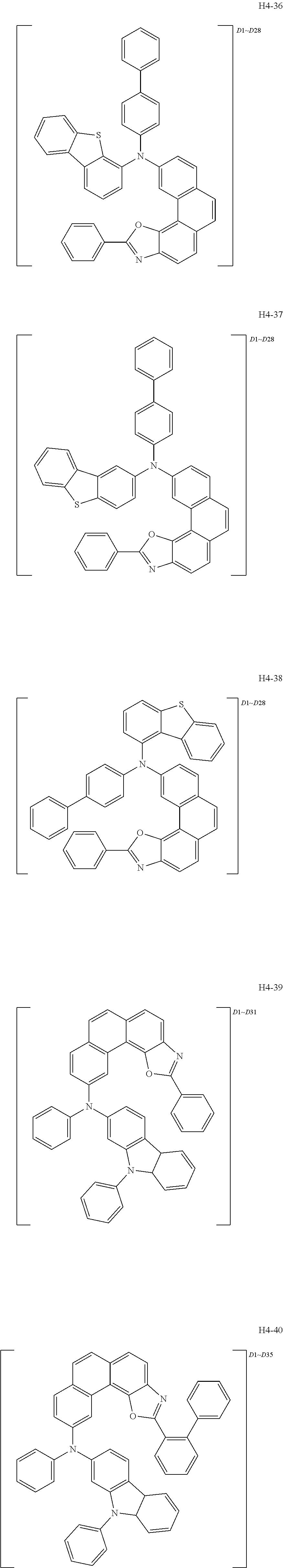

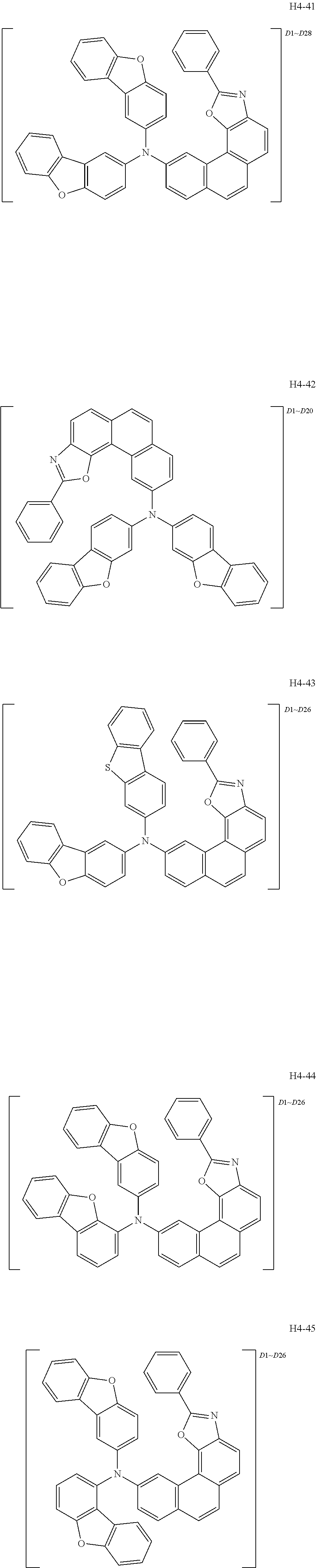

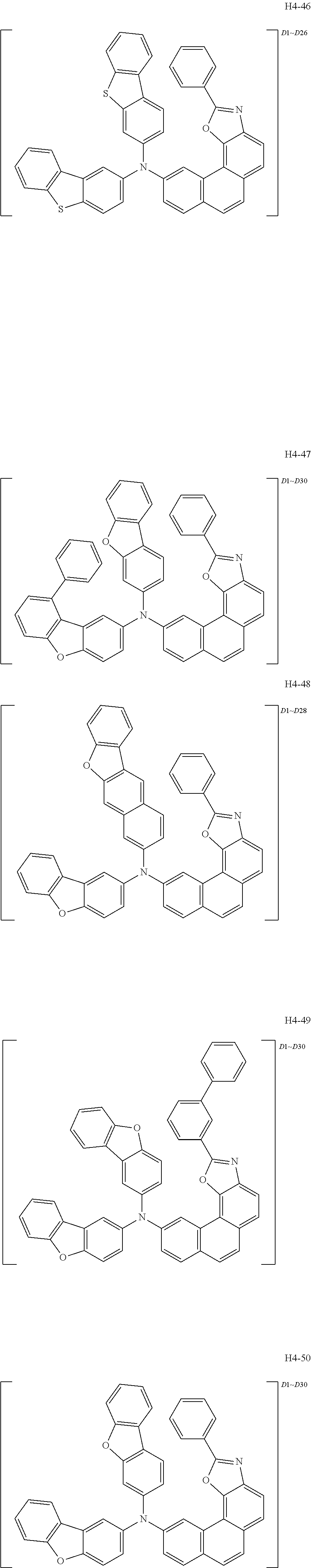

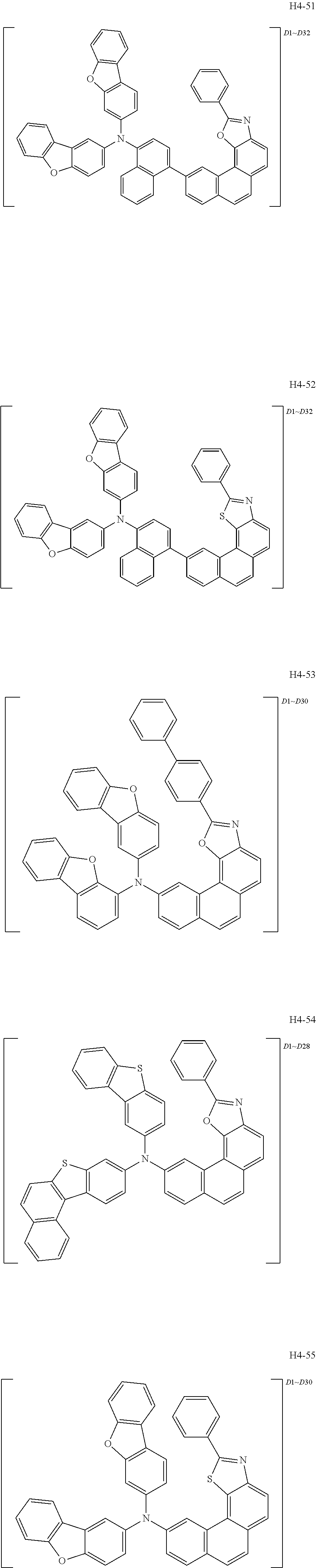

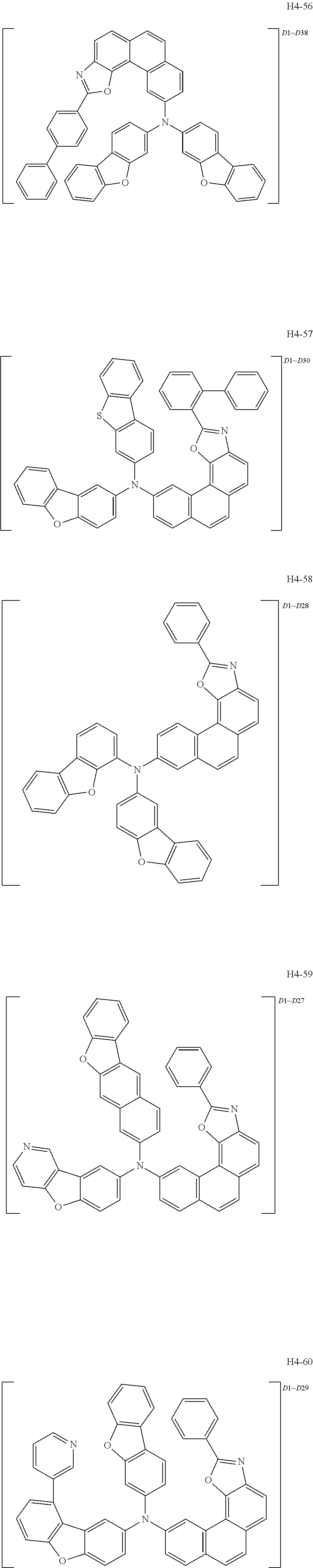

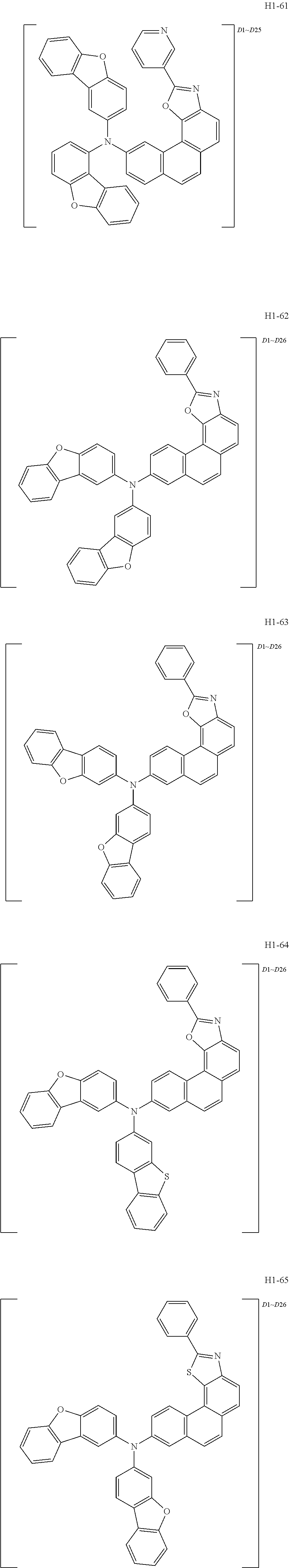

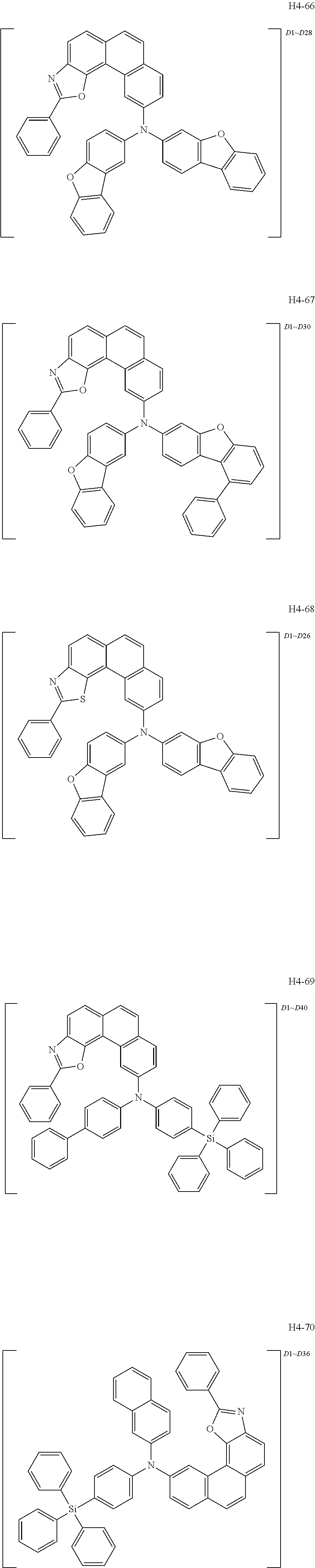

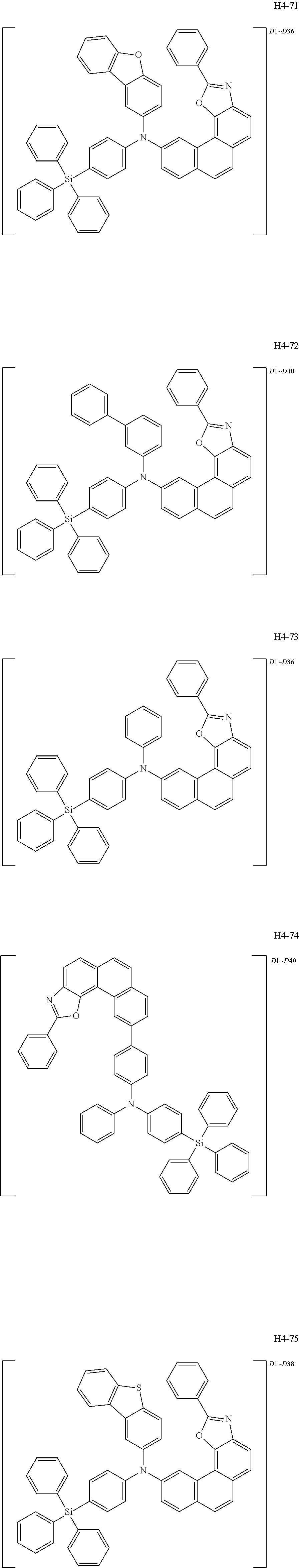

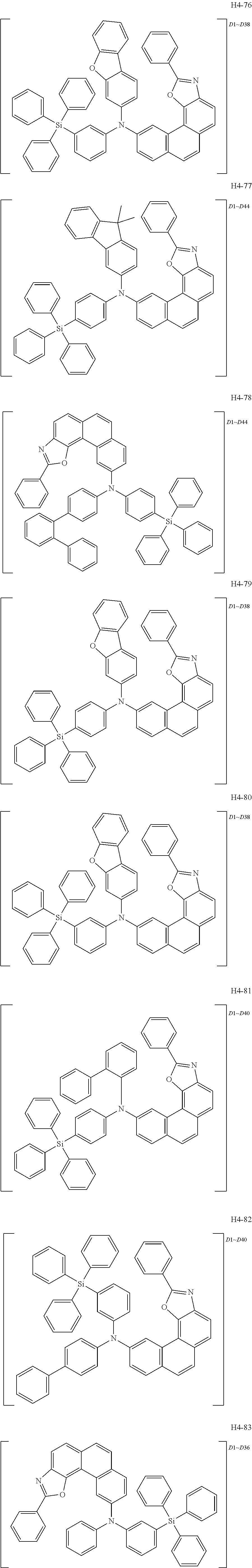

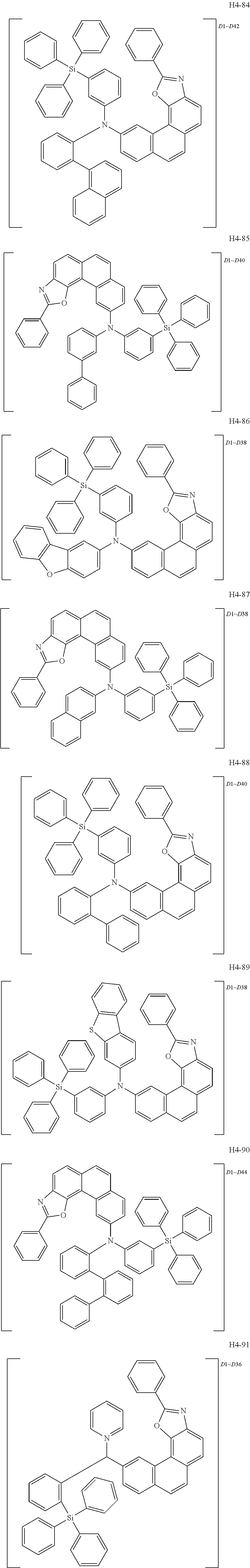

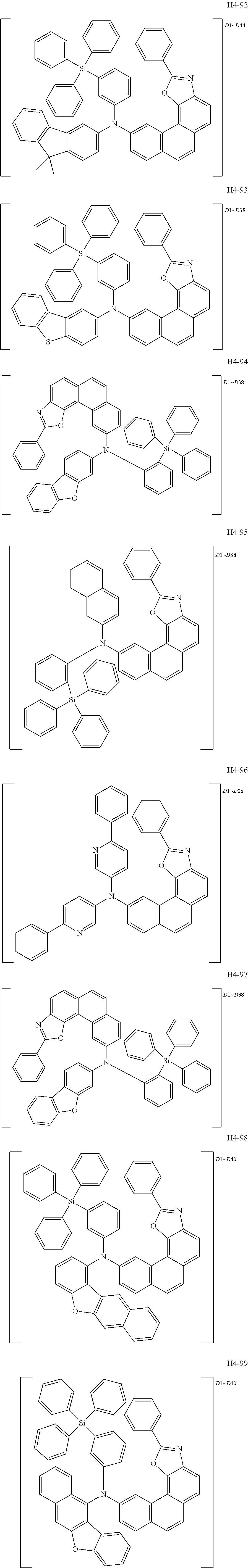

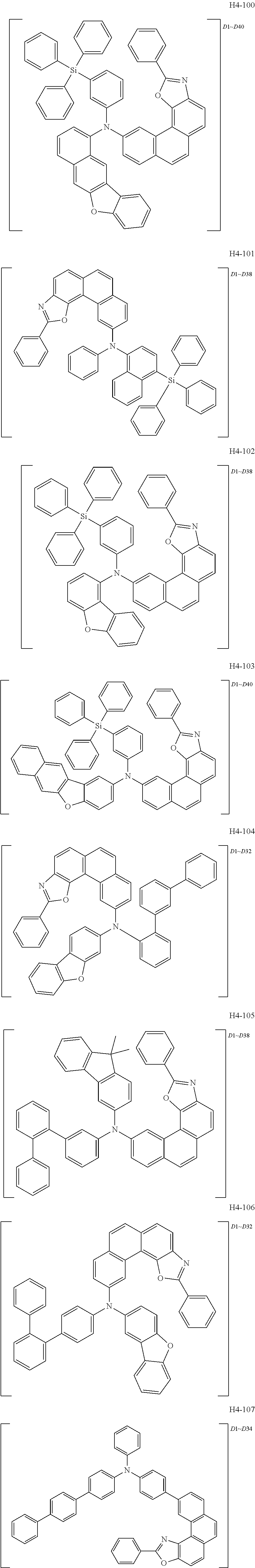

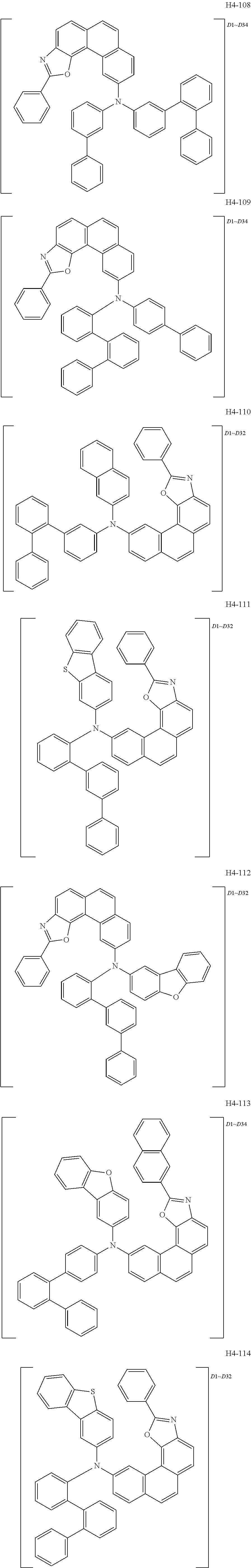

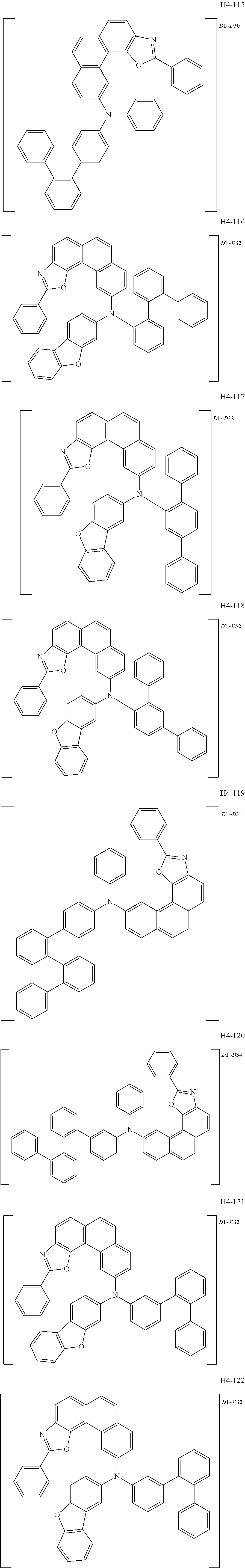

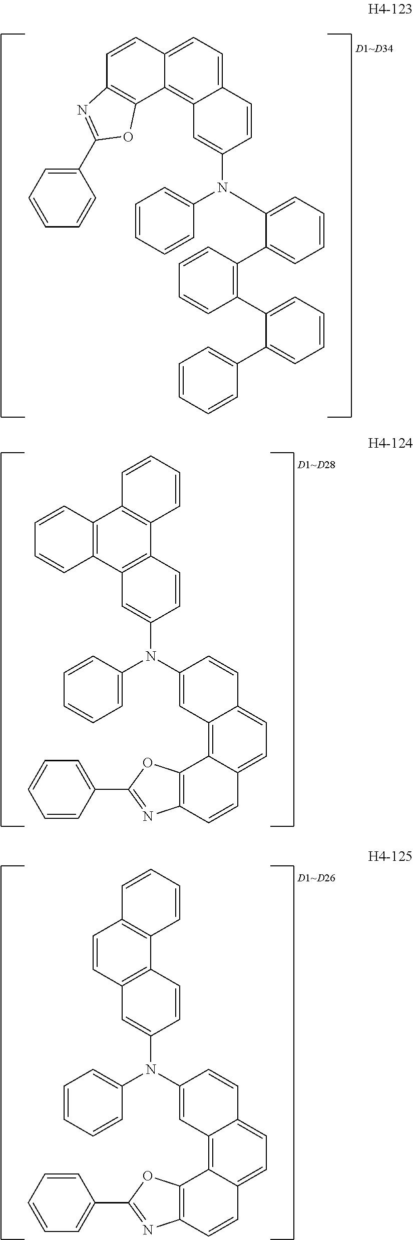

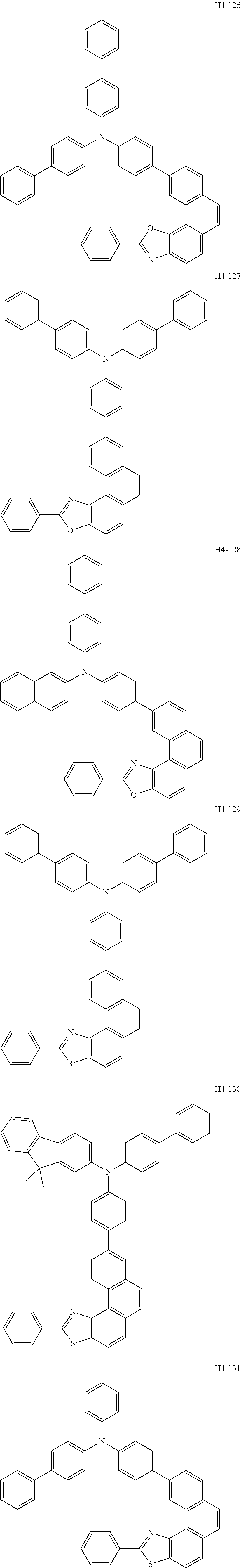

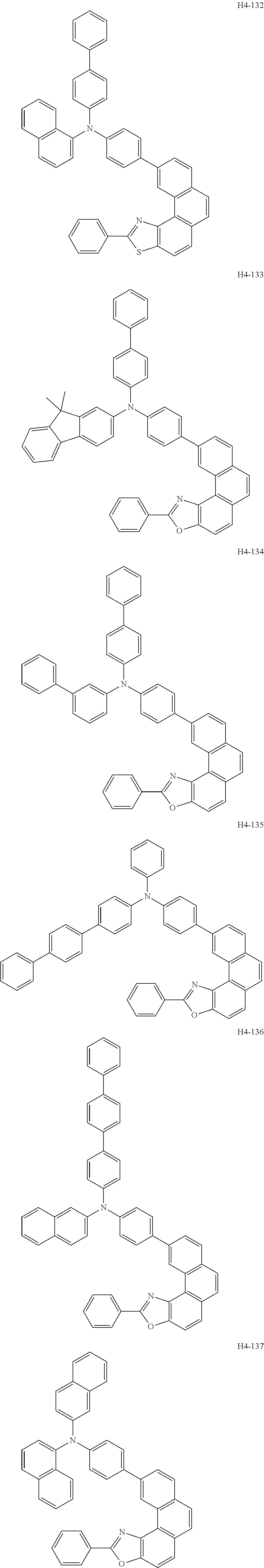

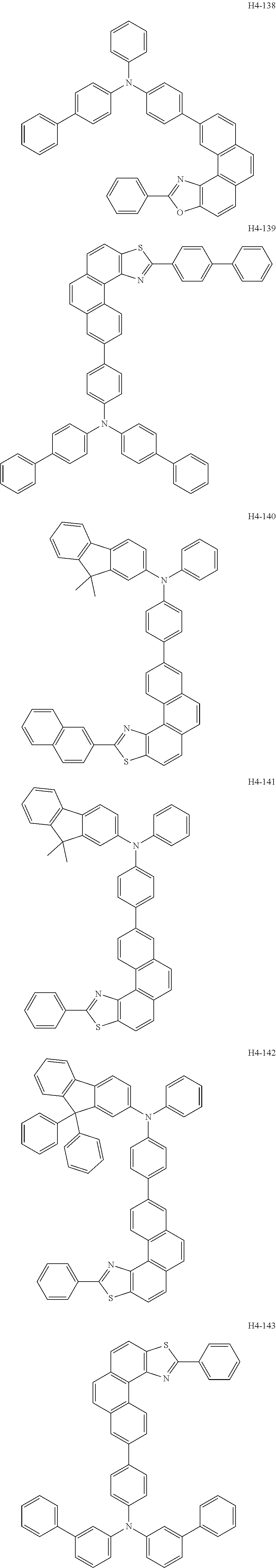

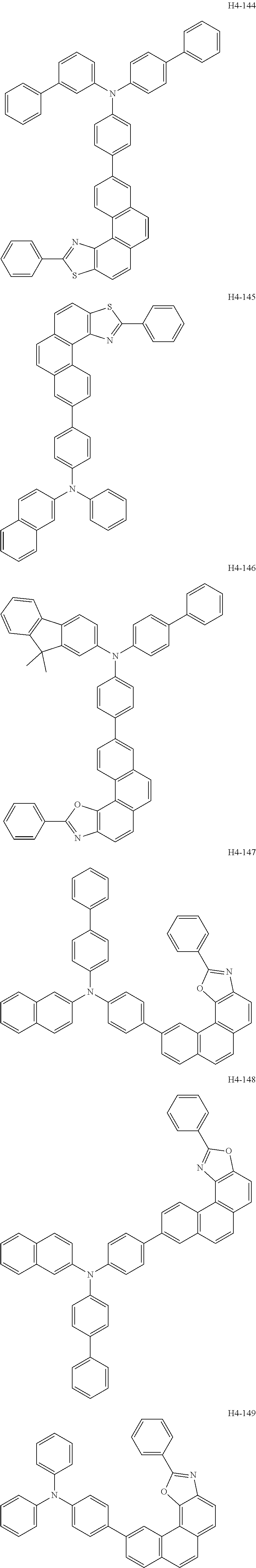

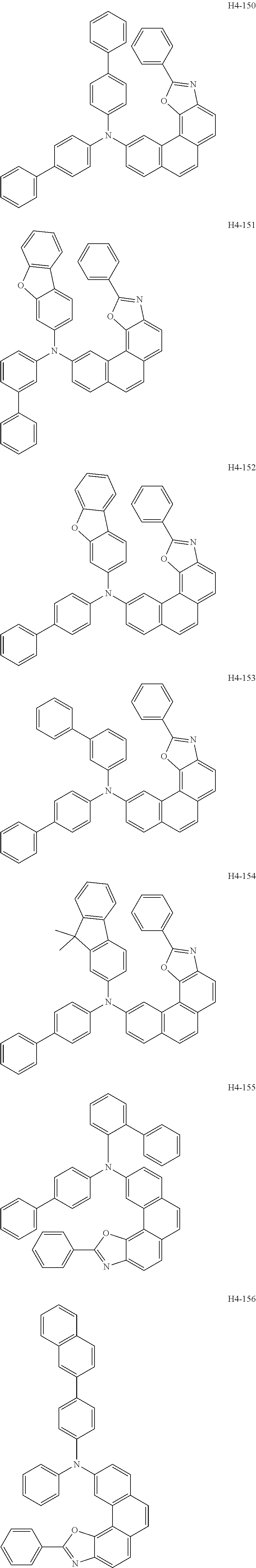

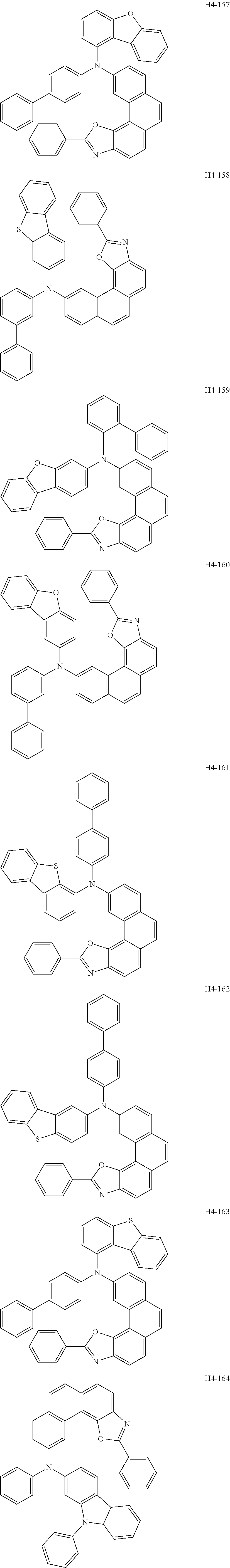

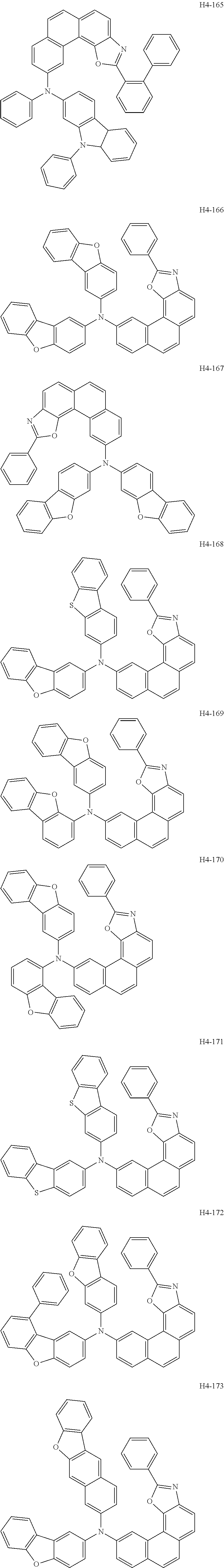

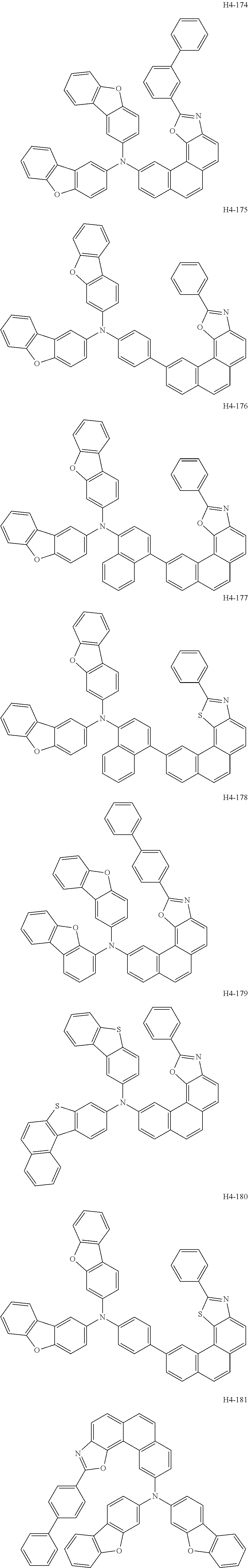

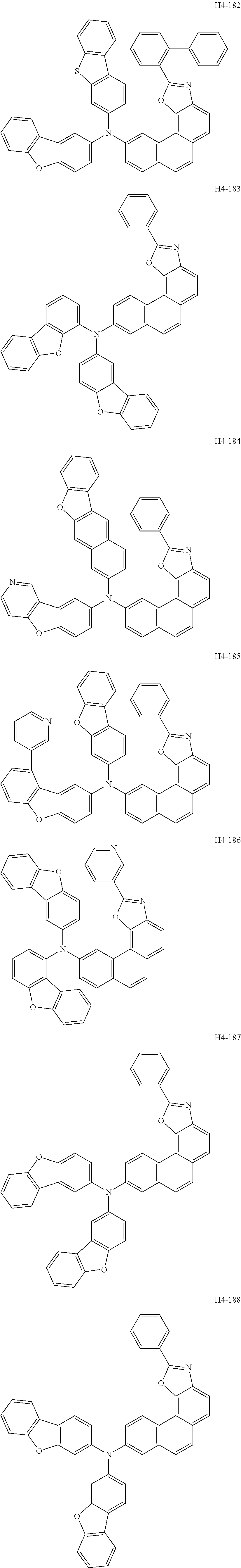

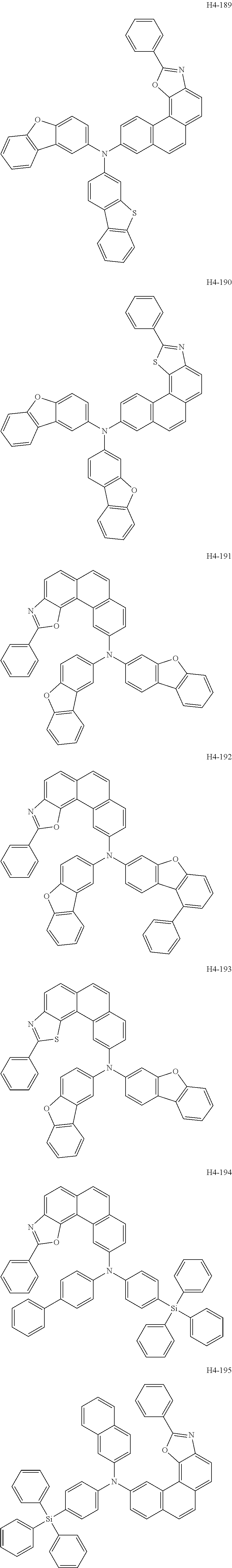

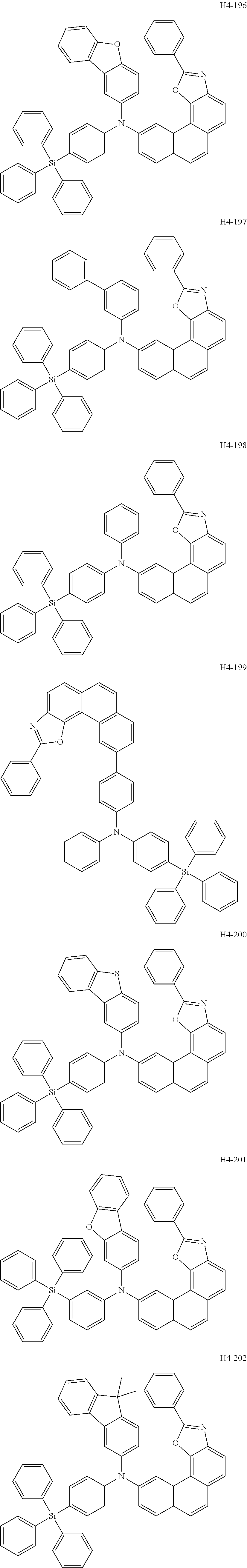

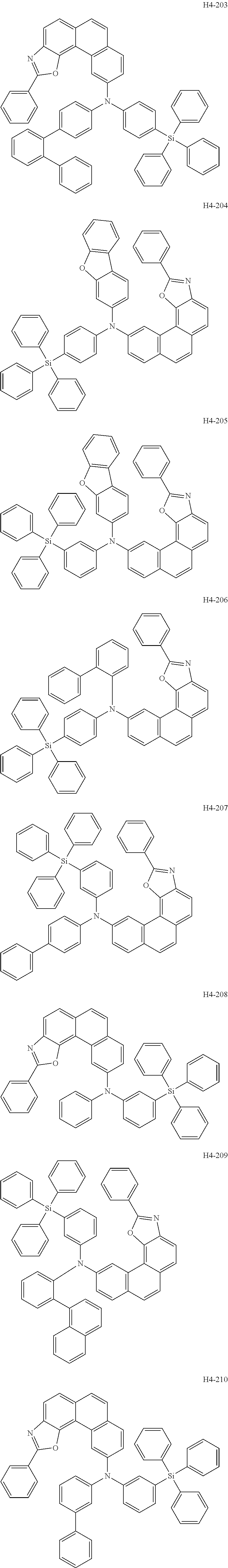

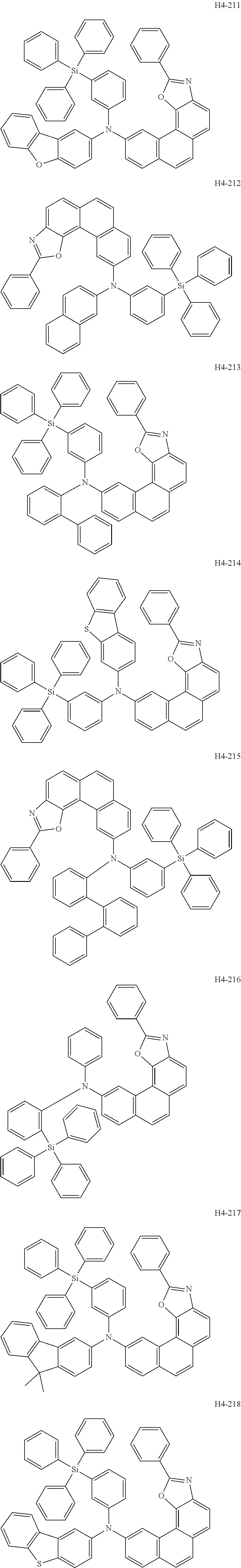

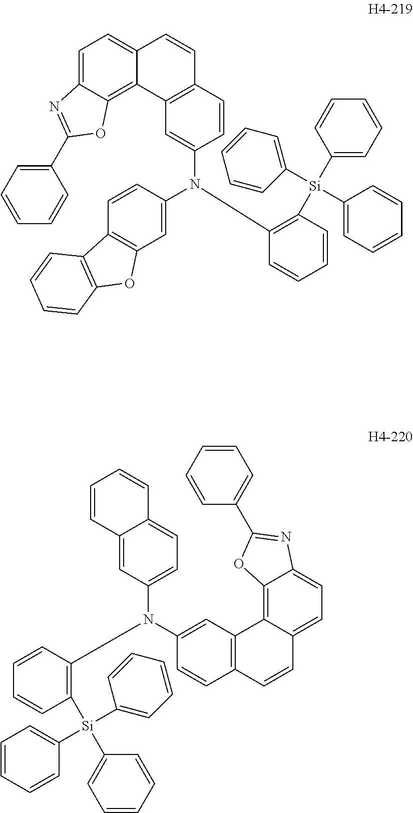 [SMILES: c1ccc(-c2nc3ccc4ccc5ccc(N(c6ccc7c(c6)oc6ccccc67)c6ccccc6[Si](c6ccccc6)(c6ccccc6)c6ccccc6)cc5c4c3o2)cc1 ; c1ccc(-c2nc3ccc4ccc5ccc(N(c6ccc7ccccc7c6)c6ccccc6[Si](c6ccccc6)(c6ccccc6)c6ccccc6)cc5c4c3o2)cc1]

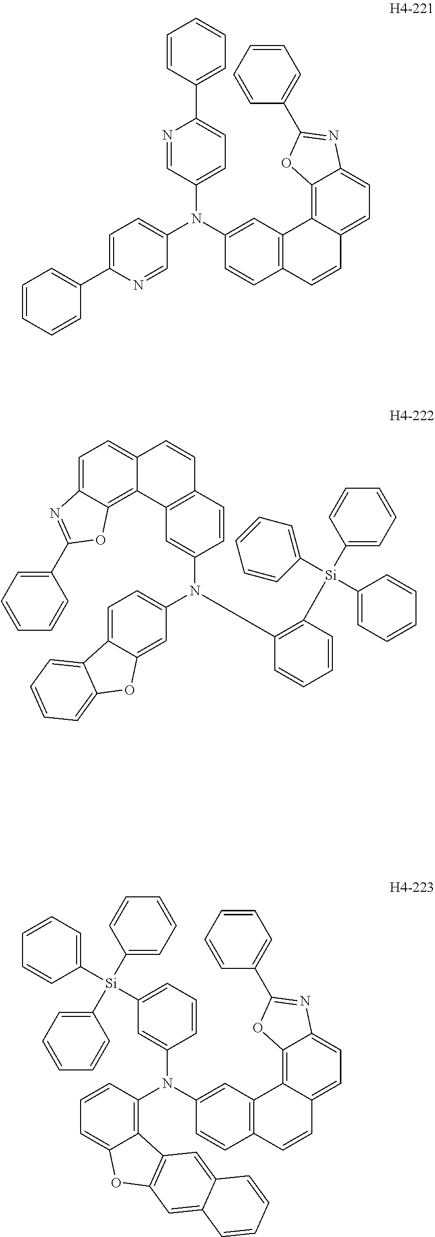 [SMILES: c1ccc(-c2ccc(N(c3ccc(-c4ccccc4)nc3)c3ccc4ccc5ccc6nc(-c7ccccc7)oc6c5c4c3)cn2)cc1 ; c1ccc(-c2nc3ccc4ccc5ccc(N(c6ccc7c(c6)oc6ccccc67)c6ccccc6[Si](c6ccccc6)(c6ccccc6)c6ccccc6)cc5c4c3o2)cc1 ; c1ccc(-c2nc3ccc4ccc5ccc(N(c6cccc([Si](c7ccccc7)(c7ccccc7)c7ccccc7)c6)c6cccc7oc8cc9ccccc9cc8c67)cc5c4c3o2)cc1]

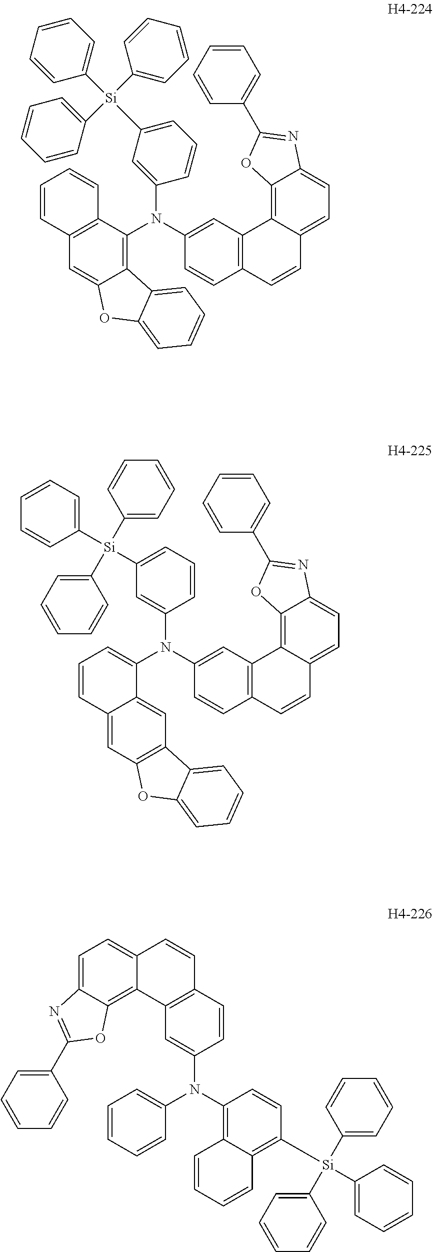 [SMILES: c1ccc(-c2nc3ccc4ccc5ccc(N(c6cccc([Si](c7ccccc7)(c7ccccc7)c7ccccc7)c6)c6c7ccccc7cc7oc8ccccc8c67)cc5c4c3o2)cc1 ; c1ccc(-c2nc3ccc4ccc5ccc(N(c6cccc([Si](c7ccccc7)(c7ccccc7)c7ccccc7)c6)c6cccc7cc8oc9ccccc9c8cc67)cc5c4c3o2)cc1 ; c1ccc(-c2nc3ccc4ccc5ccc(N(c6ccccc6)c6ccc([Si](c7ccccc7)(c7ccccc7)c7ccccc7)c7ccccc67)cc5c4c3o2)cc1]

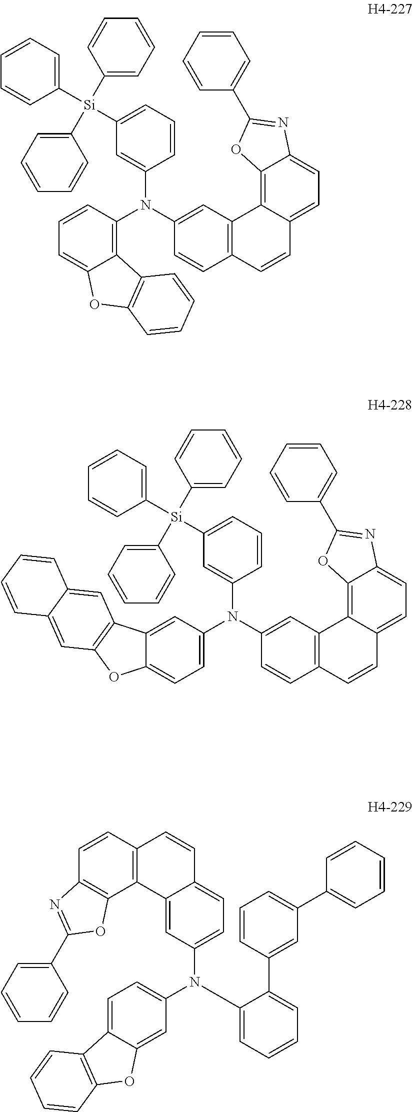 [SMILES: c1ccc(-c2nc3ccc4ccc5ccc(N(c6cccc([Si](c7ccccc7)(c7ccccc7)c7ccccc7)c6)c6cccc7oc8ccccc8c67)cc5c4c3o2)cc1 ; c1ccc(-c2nc3ccc4ccc5ccc(N(c6cccc([Si](c7ccccc7)(c7ccccc7)c7ccccc7)c6)c6ccc7oc8cc9ccccc9cc8c7c6)cc5c4c3o2)cc1 ; c1ccc(-c2cccc(-c3ccccc3N(c3ccc4c(c3)oc3ccccc34)c3ccc4ccc5ccc6nc(-c7ccccc7)oc6c5c4c3)c2)cc1]

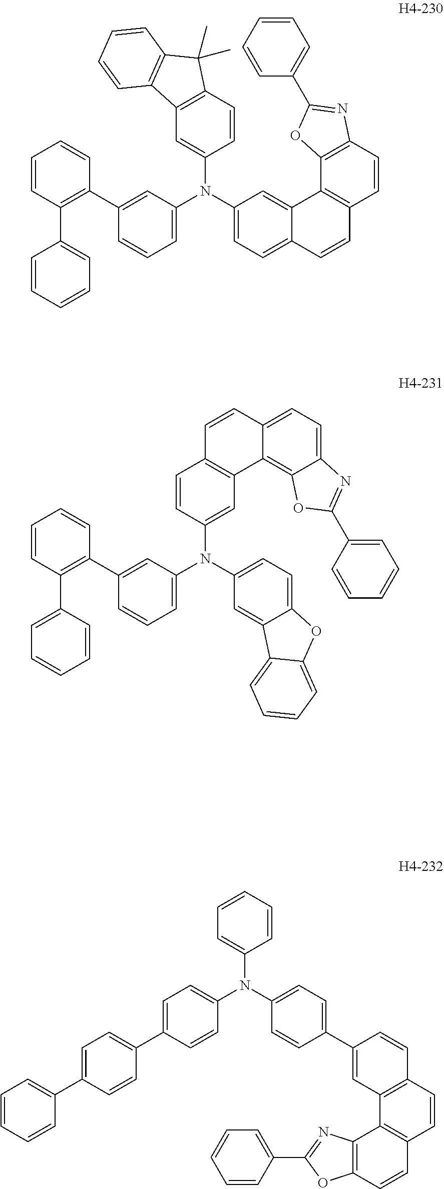 [SMILES: CC1(C)c2ccccc2-c2cc(N(c3cccc(-c4ccccc4-c4ccccc4)c3)c3ccc4ccc5ccc6nc(-c7ccccc7)oc6c5c4c3)ccc21 ; c1ccc(-c2nc3ccc4ccc5ccc(N(c6cccc(-c7ccccc7-c7ccccc7)c6)c6ccc7oc8ccccc8c7c6)cc5c4c3o2)cc1 ; c1ccc(-c2ccc(-c3ccc(N(c4ccccc4)c4ccc(-c5ccc6ccc7ccc8oc(-c9ccccc9)nc8c7c6c5)cc4)cc3)cc2)cc1]

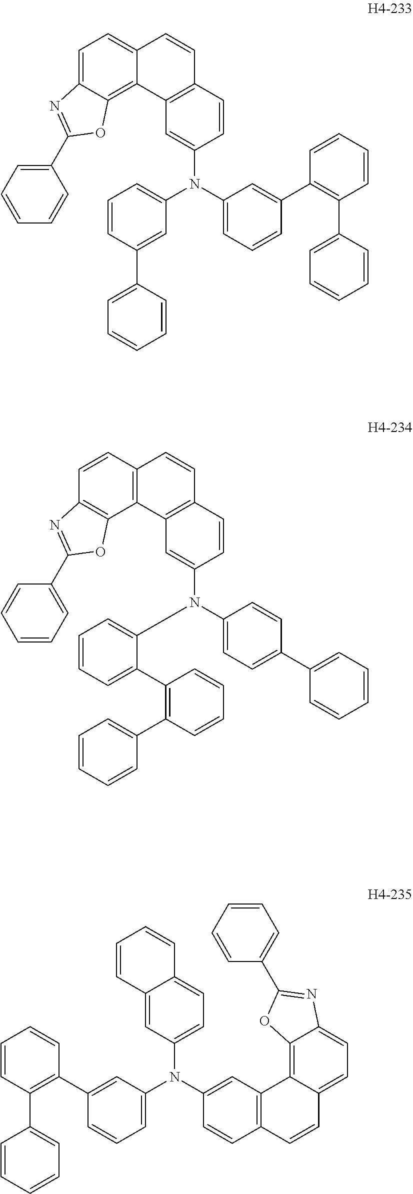 [SMILES: c1ccc(-c2cccc(N(c3cccc(-c4ccccc4-c4ccccc4)c3)c3ccc4ccc5ccc6nc(-c7ccccc7)oc6c5c4c3)c2)cc1 ; c1ccc(-c2ccc(N(c3ccc4ccc5ccc6nc(-c7ccccc7)oc6c5c4c3)c3ccccc3-c3ccccc3-c3ccccc3)cc2)cc1 ; c1ccc(-c2nc3ccc4ccc5ccc(N(c6cccc(-c7ccccc7-c7ccccc7)c6)c6ccc7ccccc7c6)cc5c4c3o2)cc1]

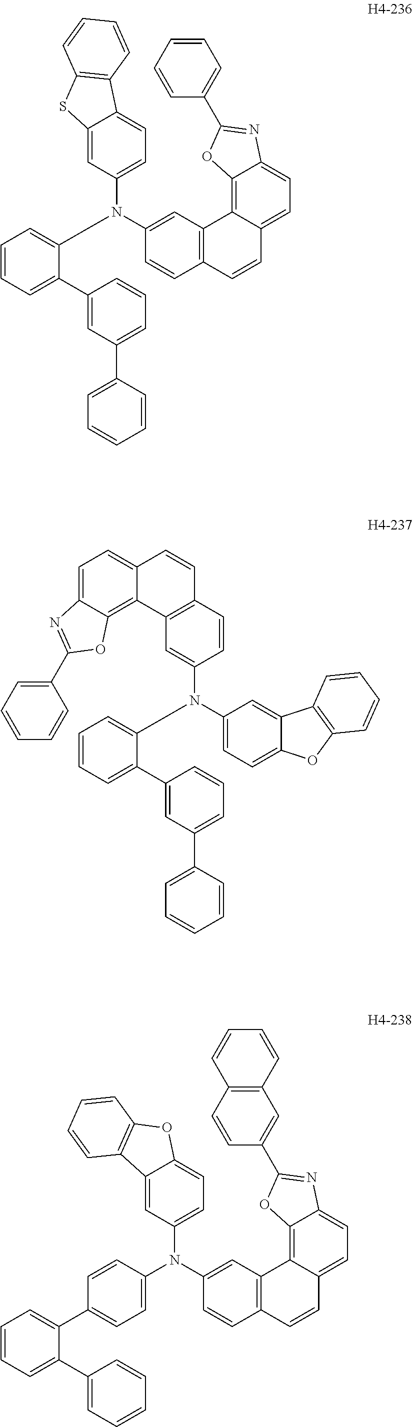 [SMILES: c1ccc(-c2cccc(-c3ccccc3N(c3ccc4c(c3)sc3ccccc34)c3ccc4ccc5ccc6nc(-c7ccccc7)oc6c5c4c3)c2)cc1 ; c1ccc(-c2cccc(-c3ccccc3N(c3ccc4oc5ccccc5c4c3)c3ccc4ccc5ccc6nc(-c7ccccc7)oc6c5c4c3)c2)cc1 ; c1ccc(-c2ccccc2-c2ccc(N(c3ccc4oc5ccccc5c4c3)c3ccc4ccc5ccc6nc(-c7ccc8ccccc8c7)oc6c5c4c3)cc2)cc1]

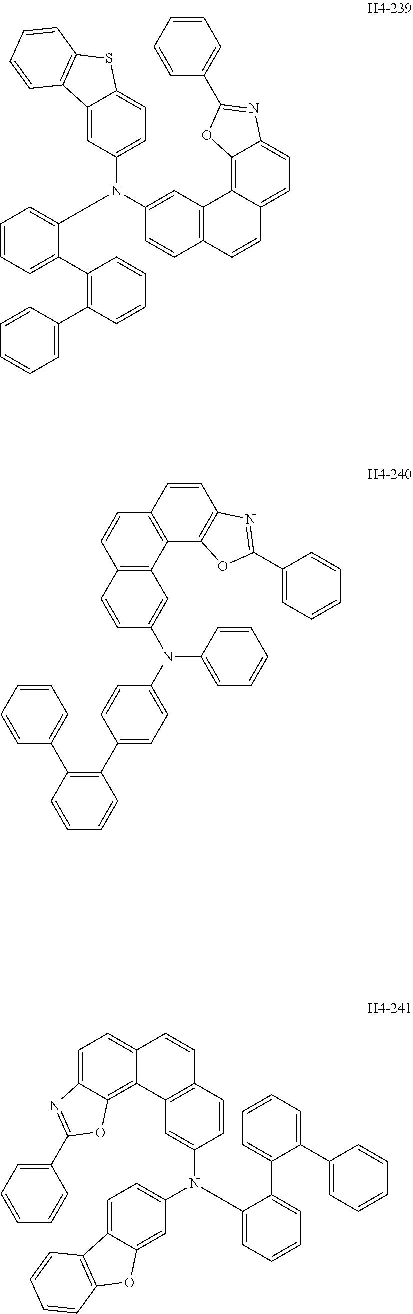 [SMILES: c1ccc(-c2nc3ccc4ccc5ccc(N(c6ccc7sc8ccccc8c7c6)c6ccccc6-c6ccccc6-c6ccccc6)cc5c4c3o2)cc1 ; c1ccc(-c2nc3ccc4ccc5ccc(N(c6ccccc6)c6ccc(-c7ccccc7-c7ccccc7)cc6)cc5c4c3o2)cc1 ; c1ccc(-c2nc3ccc4ccc5ccc(N(c6ccc7c(c6)oc6ccccc67)c6ccccc6-c6ccccc6-c6ccccc6)cc5c4c3o2)cc1]

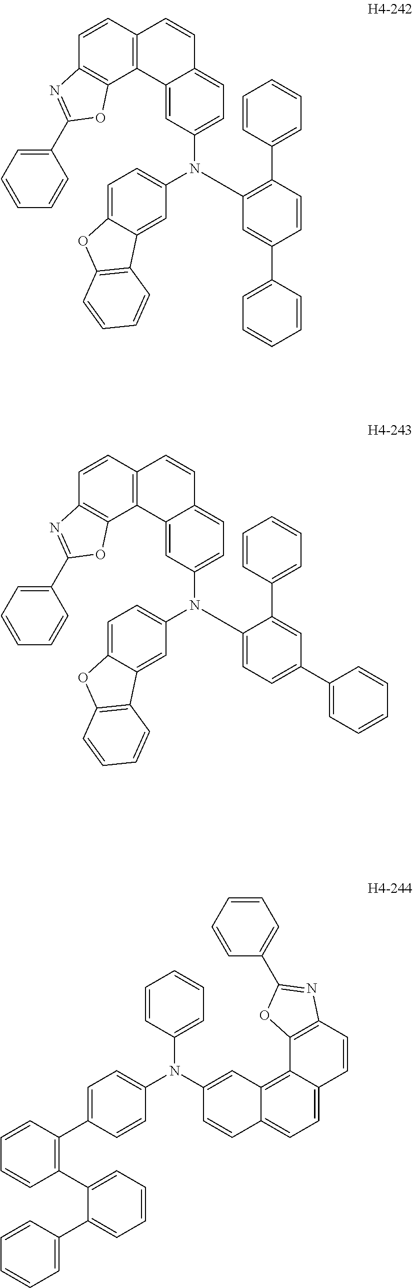 [SMILES: c1ccc(-c2ccc(-c3ccccc3)c(N(c3ccc4oc5ccccc5c4c3)c3ccc4ccc5ccc6nc(-c7ccccc7)oc6c5c4c3)c2)cc1 ; c1ccc(-c2ccc(N(c3ccc4oc5ccccc5c4c3)c3ccc4ccc5ccc6nc(-c7ccccc7)oc6c5c4c3)c(-c3ccccc3)c2)cc1 ; c1ccc(-c2nc3ccc4ccc5ccc(N(c6ccccc6)c6ccc(-c7ccccc7-c7ccccc7-c7ccccc7)cc6)cc5c4c3o2)cc1]

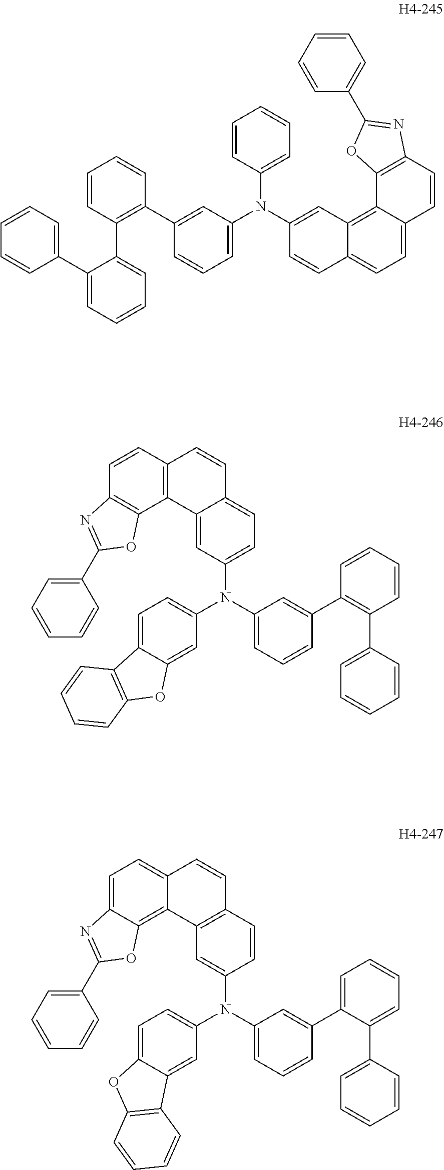 [SMILES: c1ccc(-c2nc3ccc4ccc5ccc(N(c6ccccc6)c6cccc(-c7ccccc7-c7ccccc7-c7ccccc7)c6)cc5c4c3o2)cc1 ; c1ccc(-c2nc3ccc4ccc5ccc(N(c6cccc(-c7ccccc7-c7ccccc7)c6)c6ccc7c(c6)oc6ccccc67)cc5c4c3o2)cc1 ; c1ccc(-c2nc3ccc4ccc5ccc(N(c6cccc(-c7ccccc7-c7ccccc7)c6)c6ccc7oc8ccccc8c7c6)cc5c4c3o2)cc1]

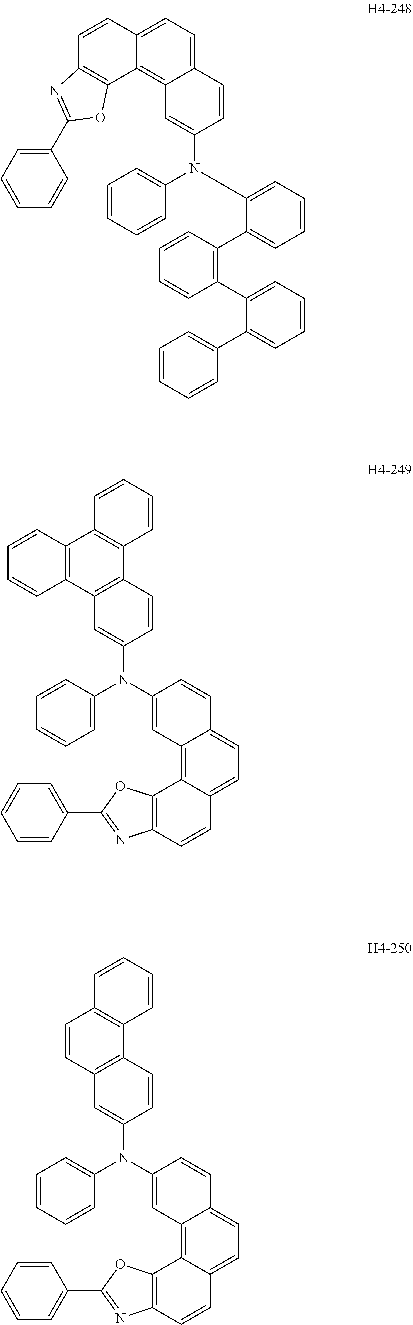 [SMILES: c1ccc(-c2nc3ccc4ccc5ccc(N(c6ccccc6)c6ccccc6-c6ccccc6-c6ccccc6-c6ccccc6)cc5c4c3o2)cc1 ; c1ccc(-c2nc3ccc4ccc5ccc(N(c6ccccc6)c6ccc7c8ccccc8c8ccccc8c7c6)cc5c4c3o2)cc1 ; c1ccc(-c2nc3ccc4ccc5ccc(N(c6ccccc6)c6ccc7c(ccc8ccccc87)c6)cc5c4c3o2)cc1]

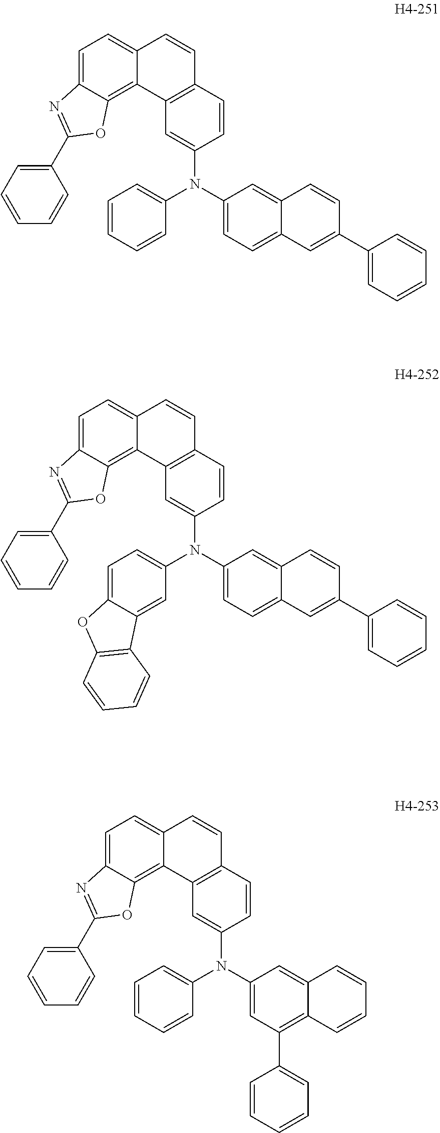 [SMILES: c1ccc(-c2ccc3cc(N(c4ccccc4)c4ccc5ccc6ccc7nc(-c8ccccc8)oc7c6c5c4)ccc3c2)cc1 ; c1ccc(-c2ccc3cc(N(c4ccc5oc6ccccc6c5c4)c4ccc5ccc6ccc7nc(-c8ccccc8)oc7c6c5c4)ccc3c2)cc1 ; c1ccc(-c2nc3ccc4ccc5ccc(N(c6ccccc6)c6cc(-c7ccccc7)c7ccccc7c6)cc5c4c3o2)cc1]

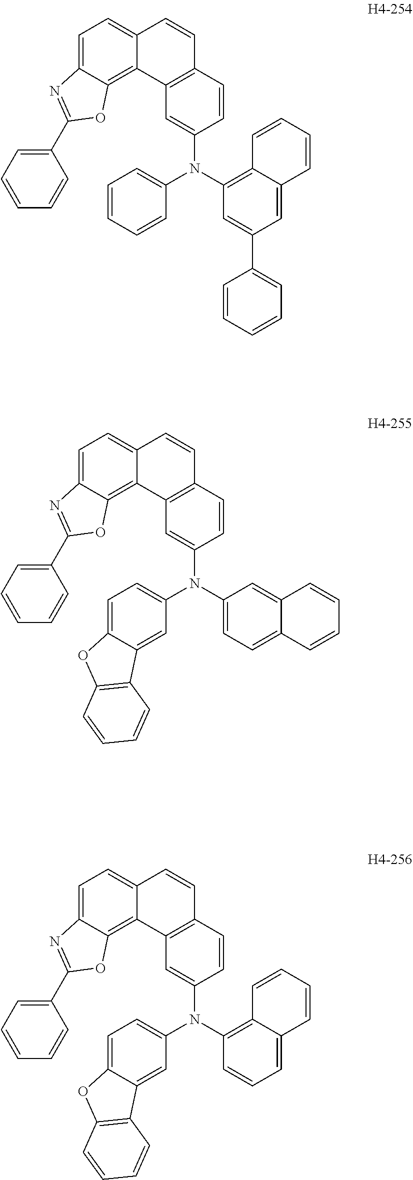 [SMILES: c1ccc(-c2cc(N(c3ccccc3)c3ccc4ccc5ccc6nc(-c7ccccc7)oc6c5c4c3)c3ccccc3c2)cc1 ; c1ccc(-c2nc3ccc4ccc5ccc(N(c6ccc7ccccc7c6)c6ccc7oc8ccccc8c7c6)cc5c4c3o2)cc1 ; c1ccc(-c2nc3ccc4ccc5ccc(N(c6ccc7oc8ccccc8c7c6)c6cccc7ccccc67)cc5c4c3o2)cc1]

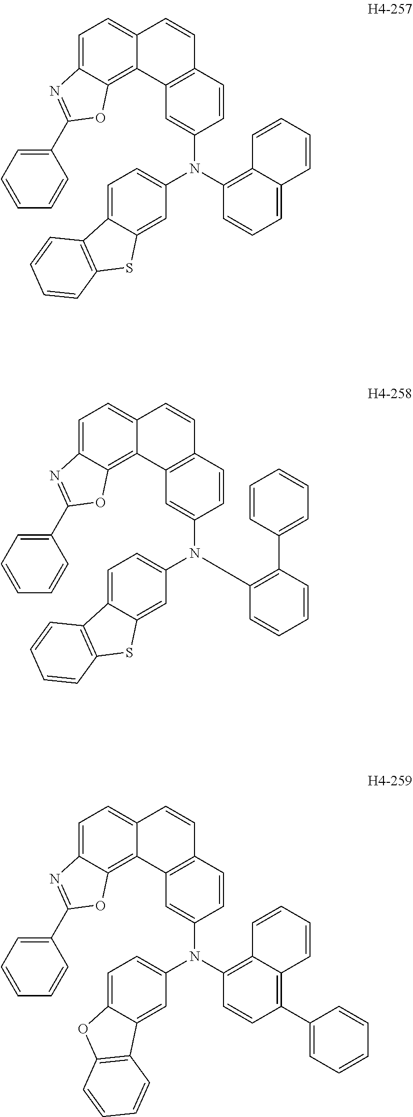 [SMILES: c1ccc(-c2nc3ccc4ccc5ccc(N(c6ccc7c(c6)sc6ccccc67)c6cccc7ccccc67)cc5c4c3o2)cc1 ; c1ccc(-c2nc3ccc4ccc5ccc(N(c6ccc7c(c6)sc6ccccc67)c6ccccc6-c6ccccc6)cc5c4c3o2)cc1 ; c1ccc(-c2nc3ccc4ccc5ccc(N(c6ccc7oc8ccccc8c7c6)c6ccc(-c7ccccc7)c7ccccc67)cc5c4c3o2)cc1]

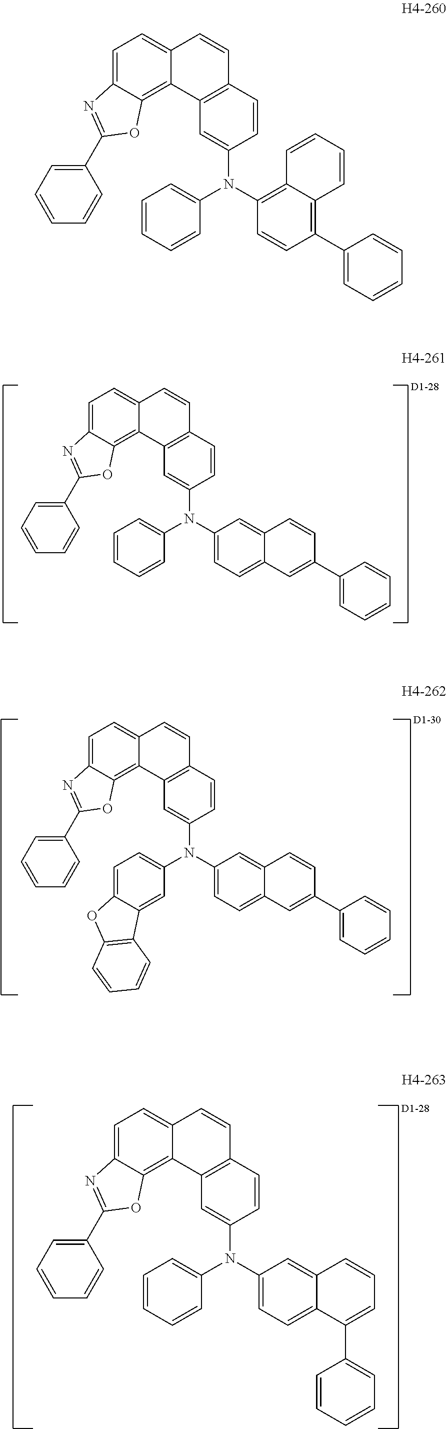

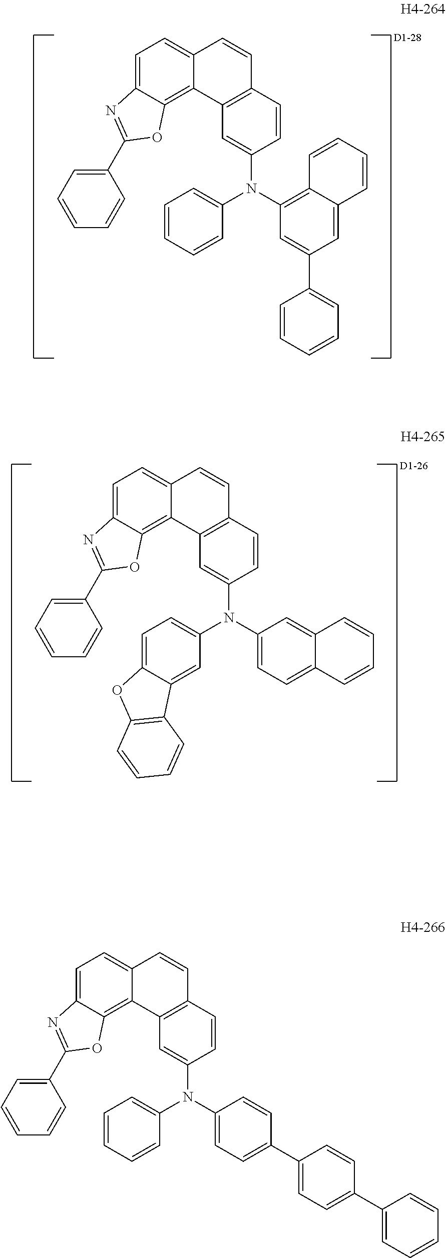

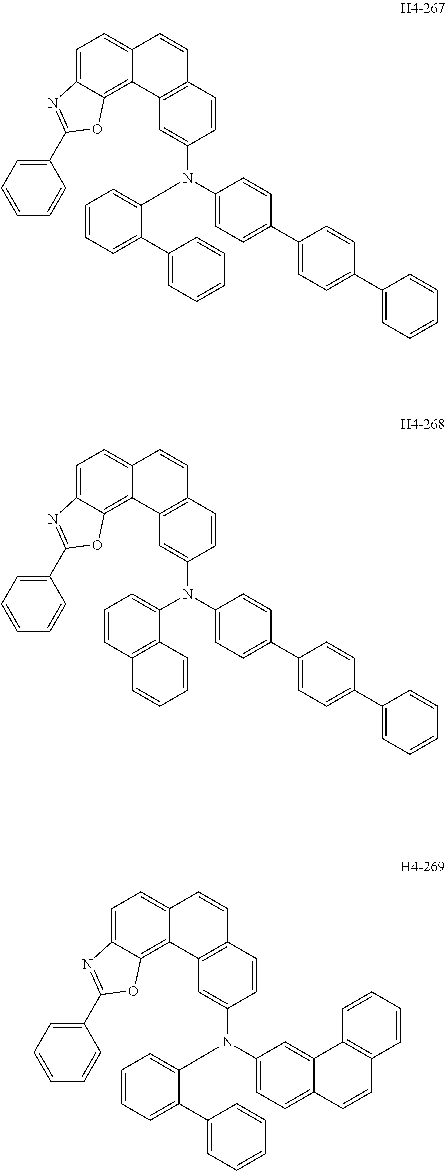 [SMILES: c1ccc(-c2ccc(-c3ccc(N(c4ccc5ccc6ccc7nc(-c8ccccc8)oc7c6c5c4)c4ccccc4-c4ccccc4)cc3)cc2)cc1 ; c1ccc(-c2ccc(-c3ccc(N(c4ccc5ccc6ccc7nc(-c8ccccc8)oc7c6c5c4)c4cccc5ccccc45)cc3)cc2)cc1 ; c1ccc(-c2nc3ccc4ccc5ccc(N(c6ccc7ccc8ccccc8c7c6)c6ccccc6-c6ccccc6)cc5c4c3o2)cc1]

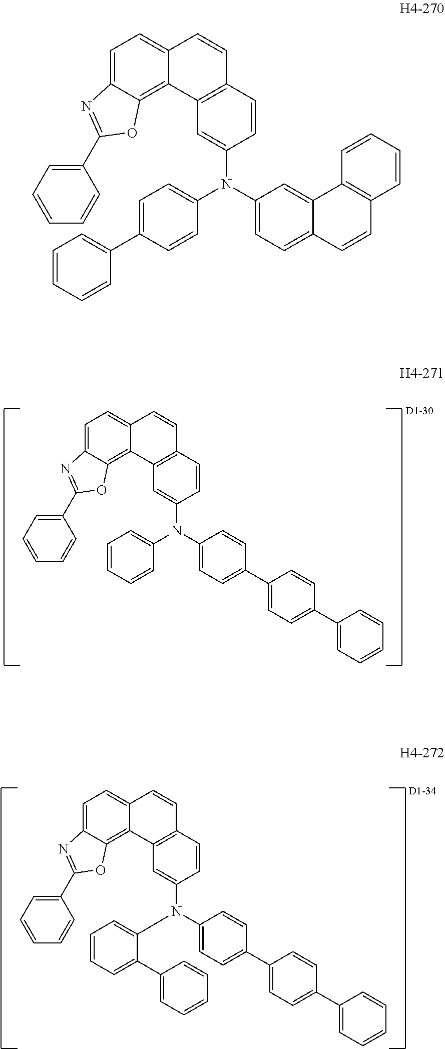

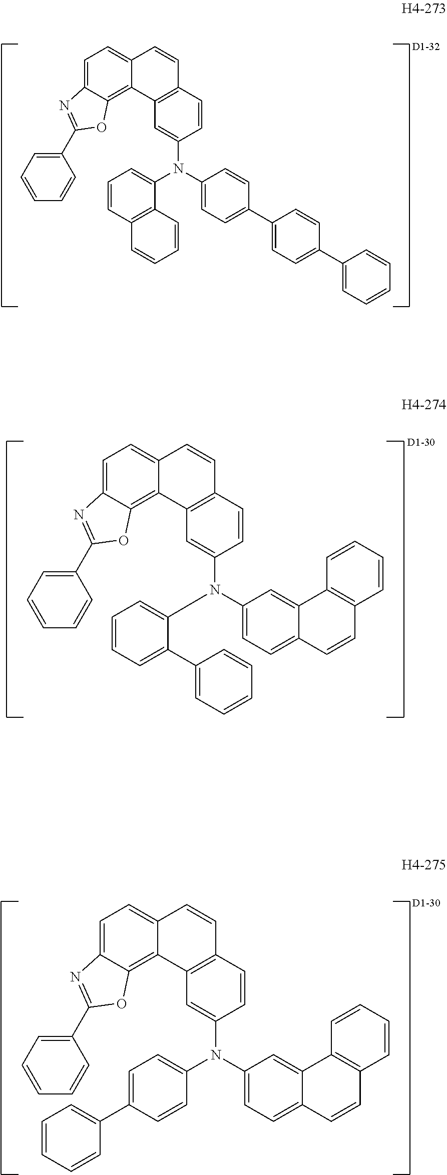

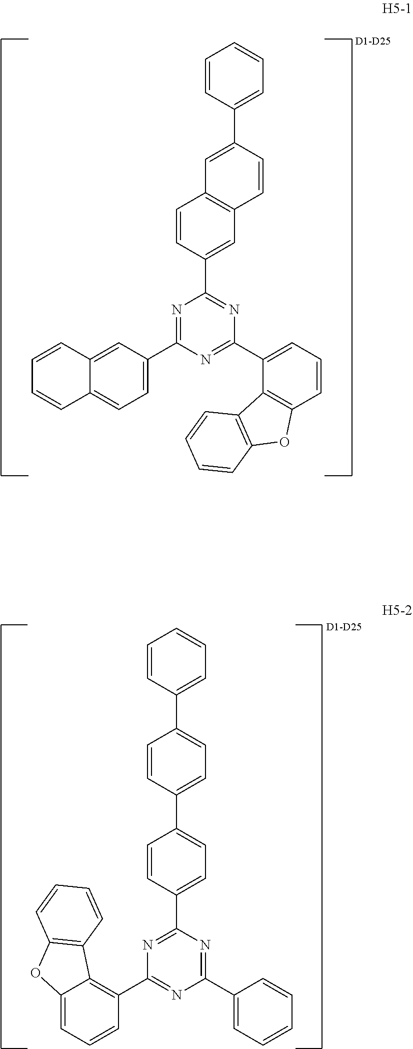

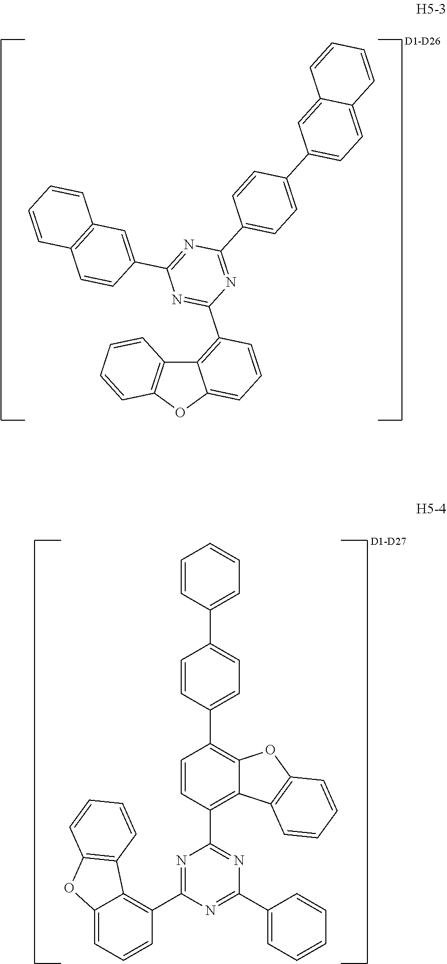

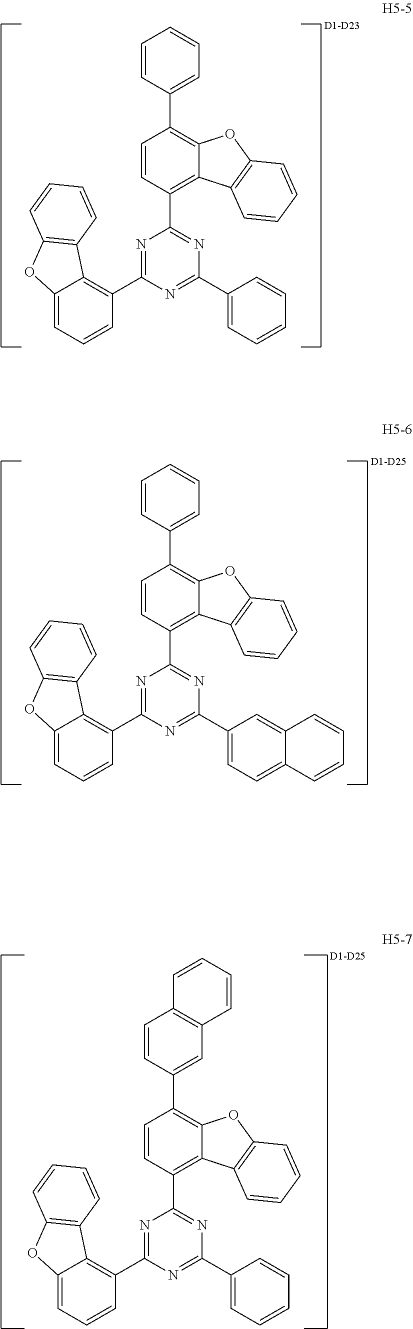

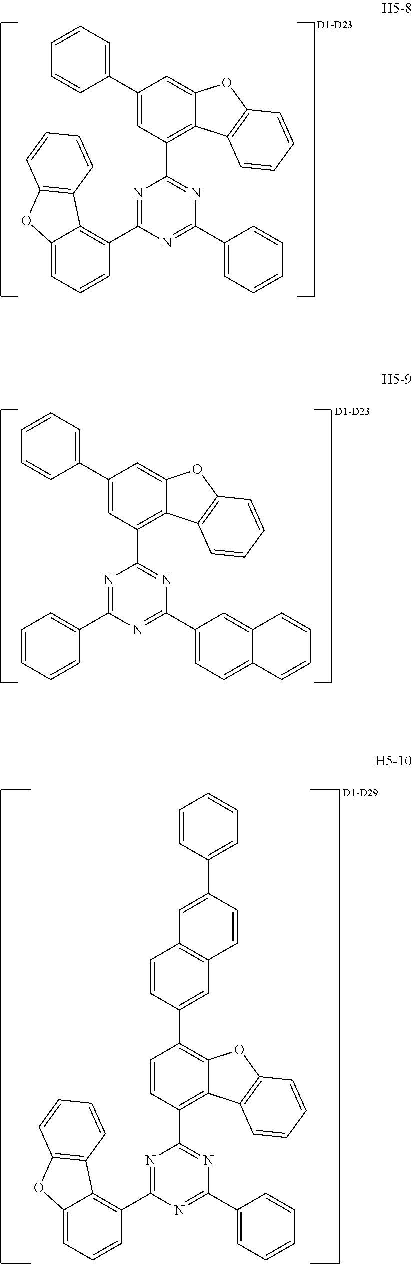

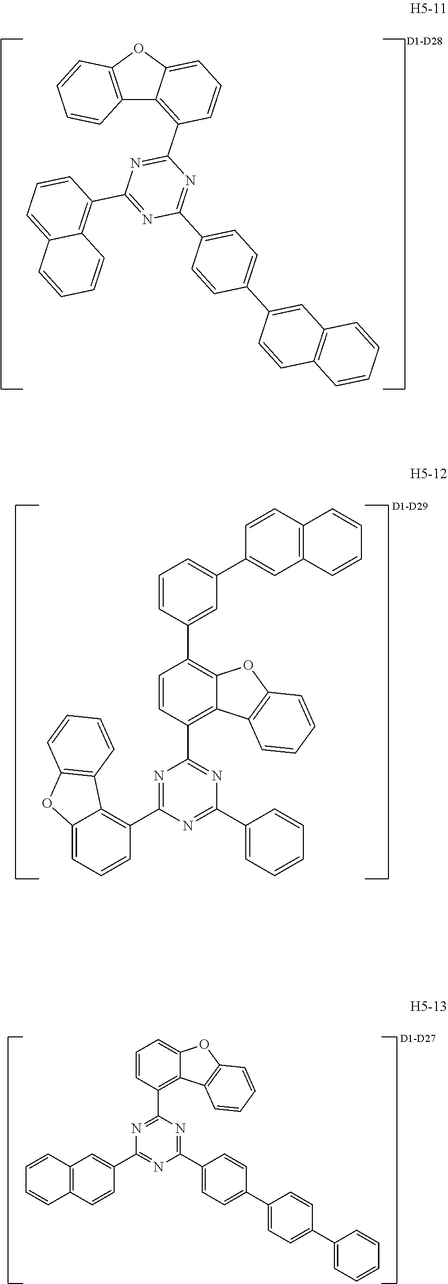

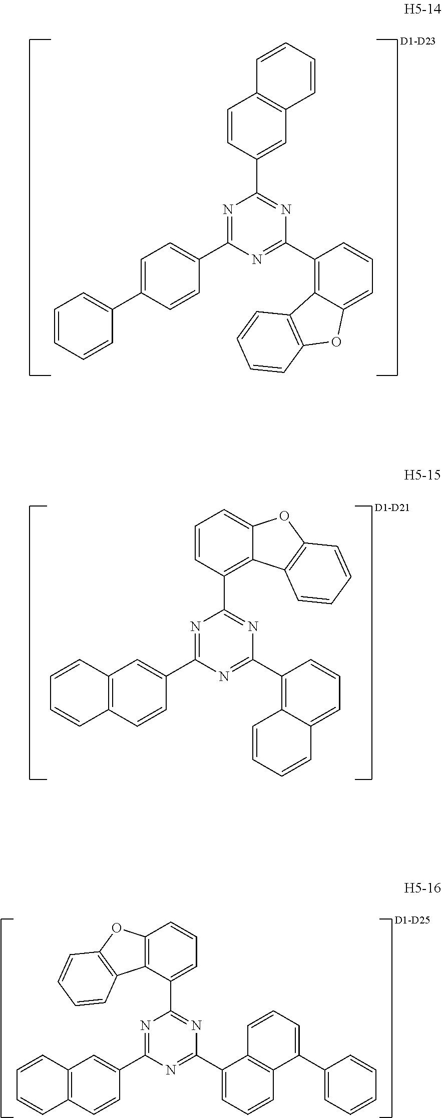

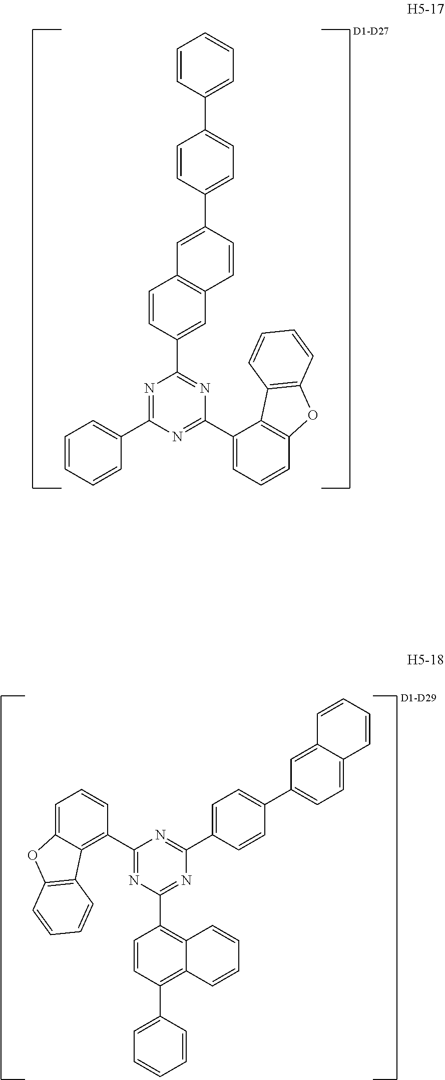

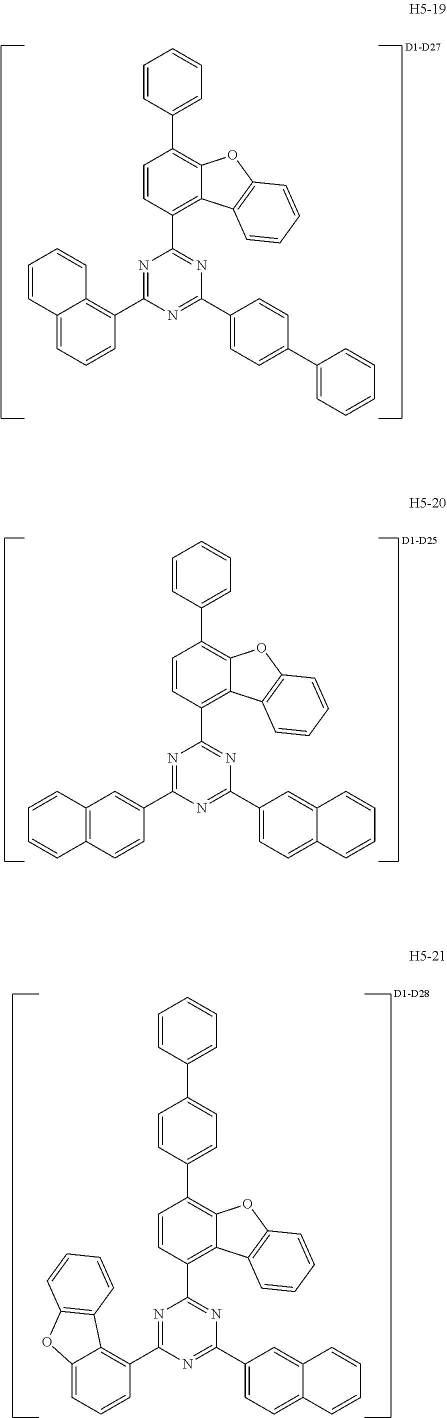

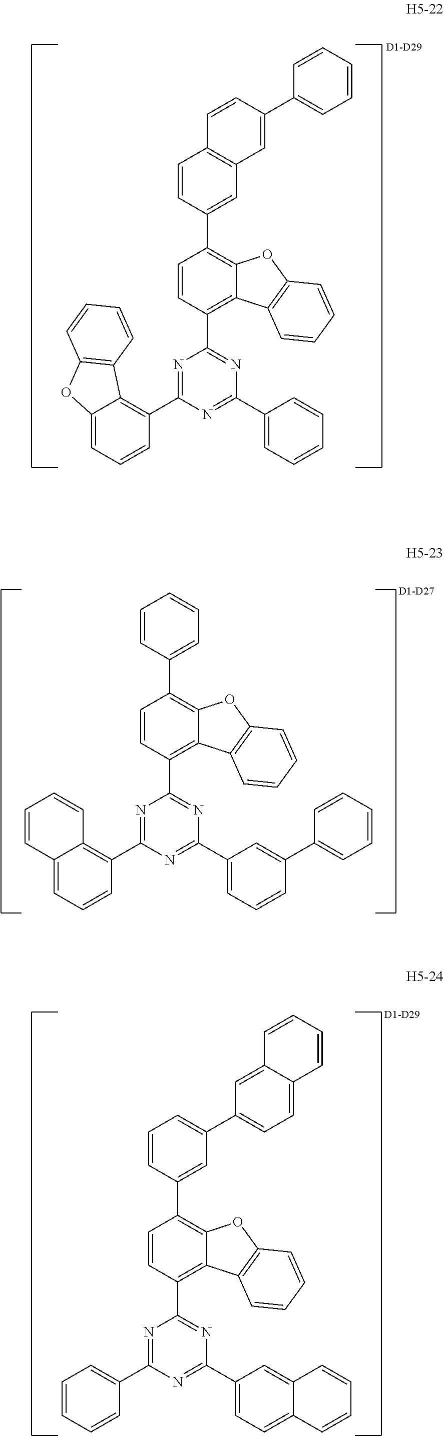

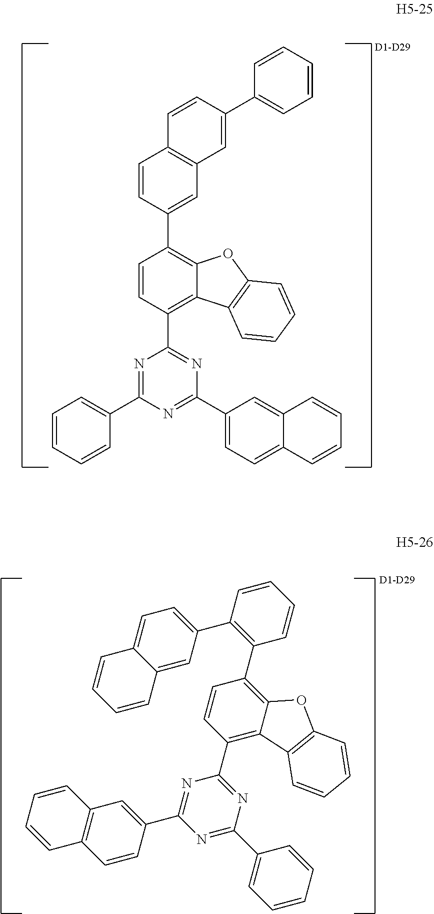

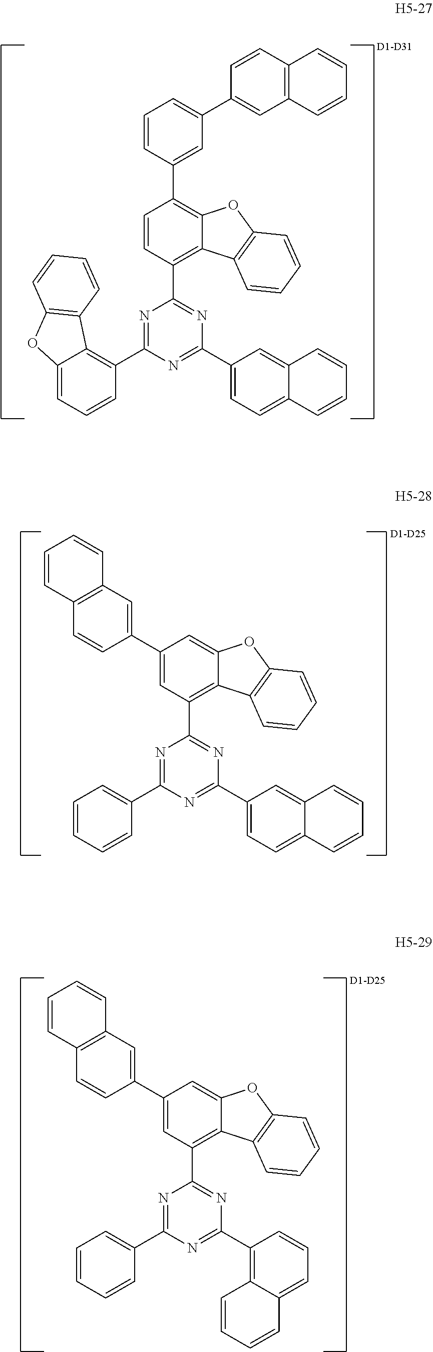

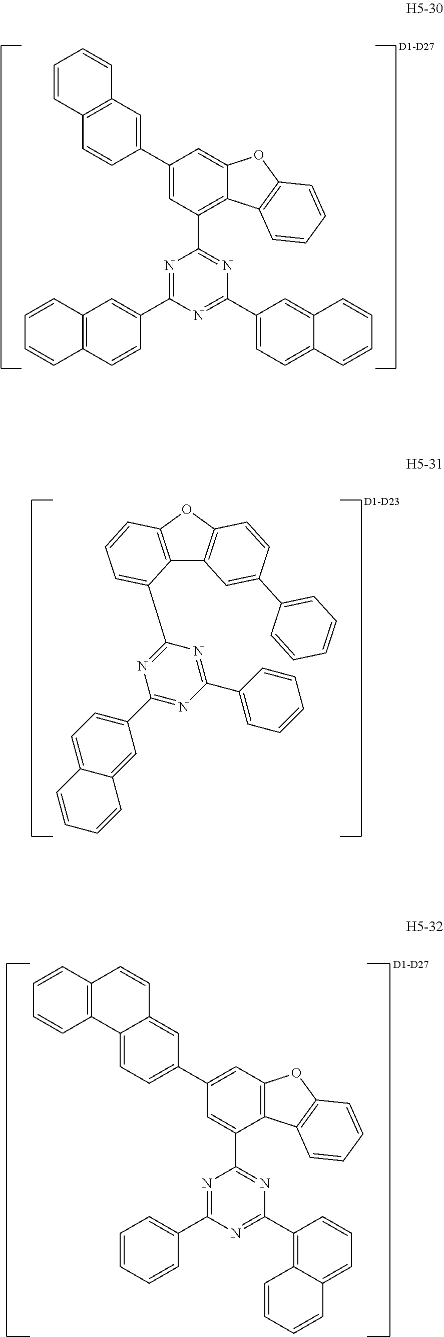

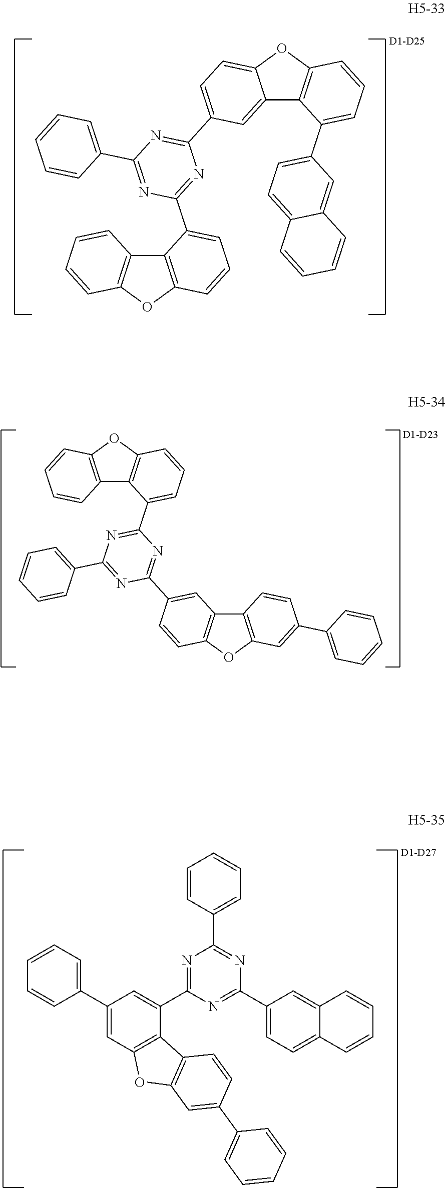

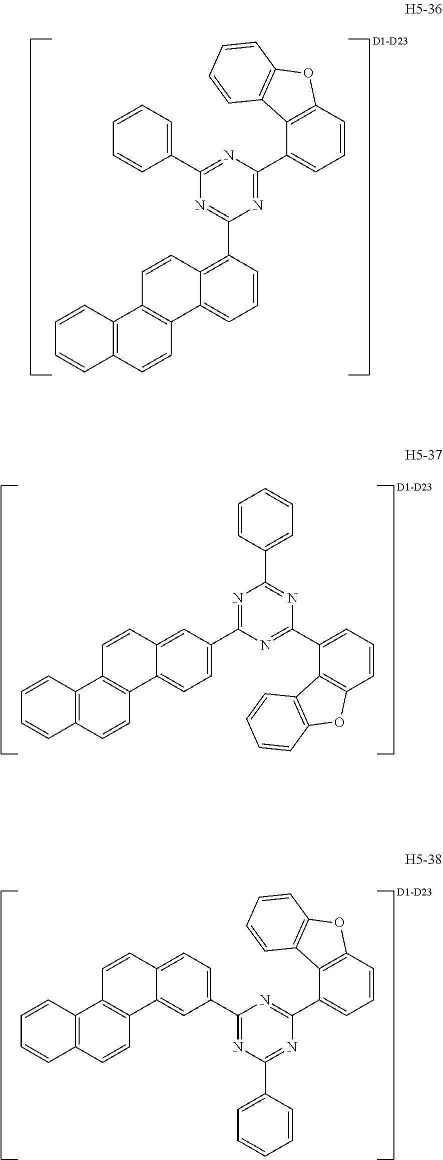

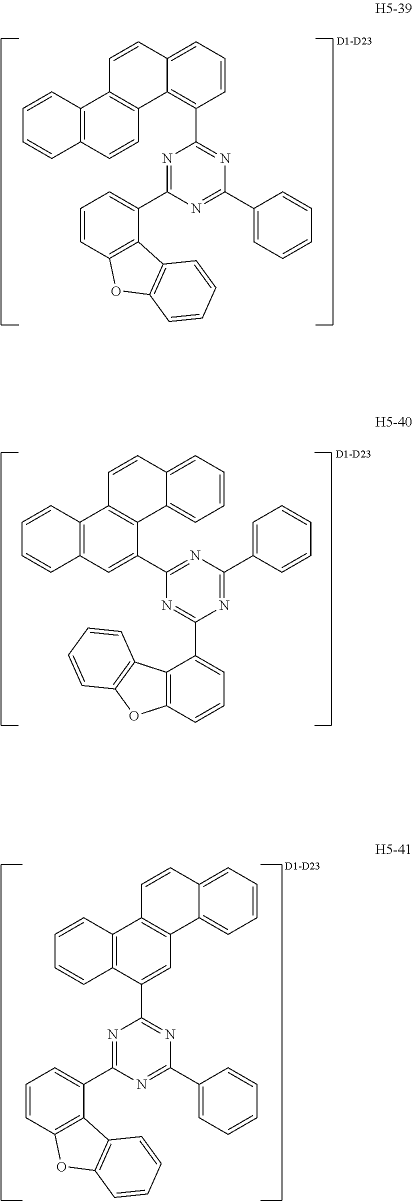

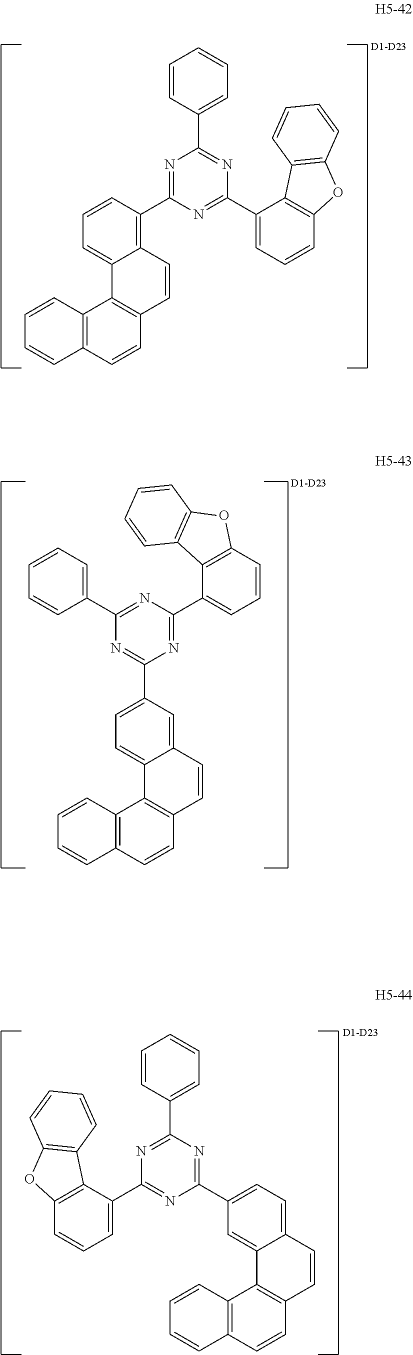

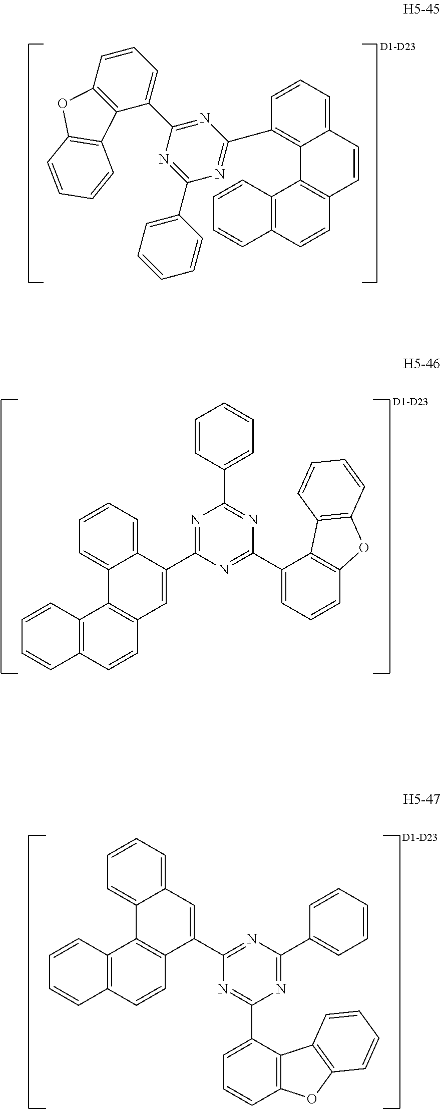

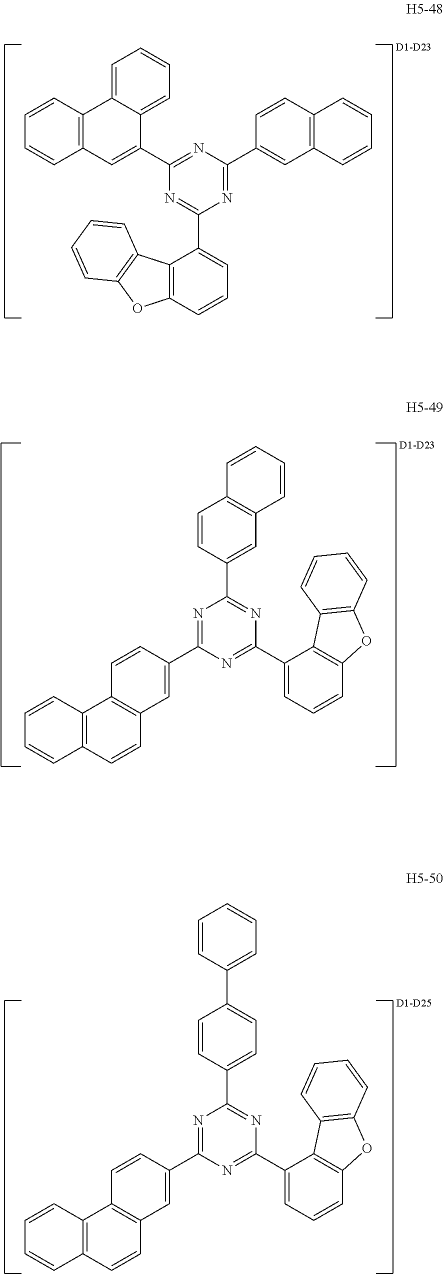

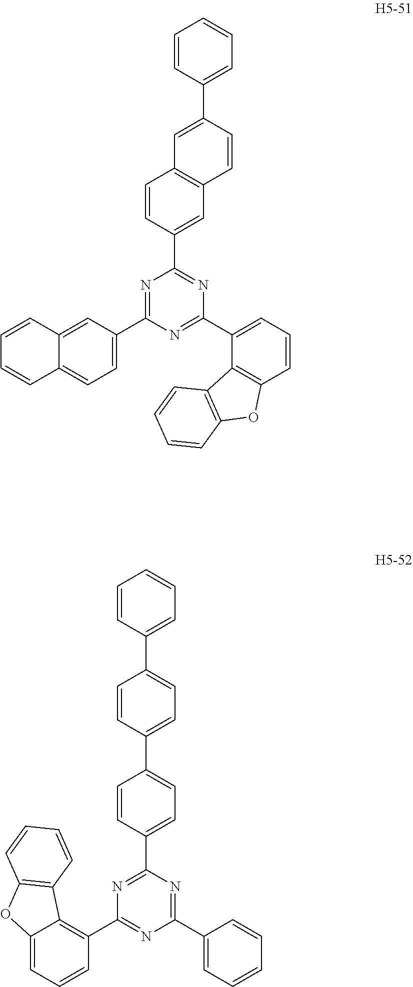 [SMILES: c1ccc(-c2ccc3cc(-c4nc(-c5ccc6ccccc6c5)nc(-c5cccc6oc7ccccc7c56)n4)ccc3c2)cc1 ; c1ccc(-c2ccc(-c3ccc(-c4nc(-c5ccccc5)nc(-c5cccc6oc7ccccc7c56)n4)cc3)cc2)cc1]

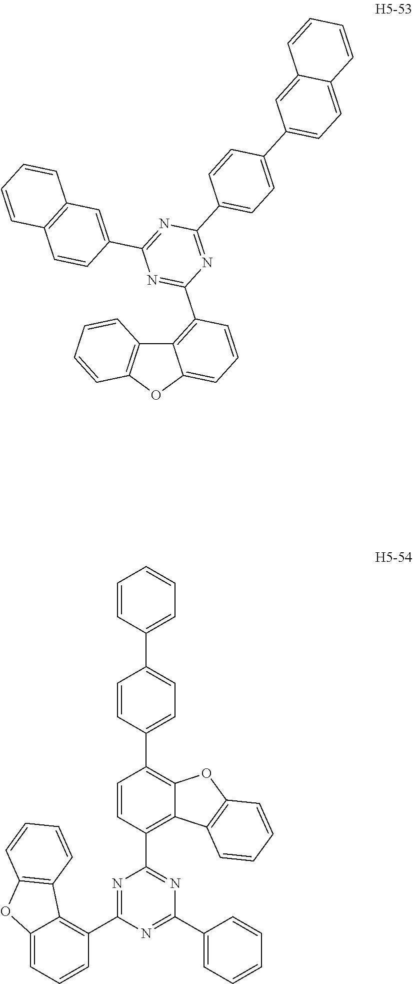 [SMILES: c1ccc2cc(-c3ccc(-c4nc(-c5ccc6ccccc6c5)nc(-c5cccc6oc7ccccc7c56)n4)cc3)ccc2c1 ; c1ccc(-c2ccc(-c3ccc(-c4nc(-c5ccccc5)nc(-c5cccc6oc7ccccc7c56)n4)c4c3oc3ccccc34)cc2)cc1]

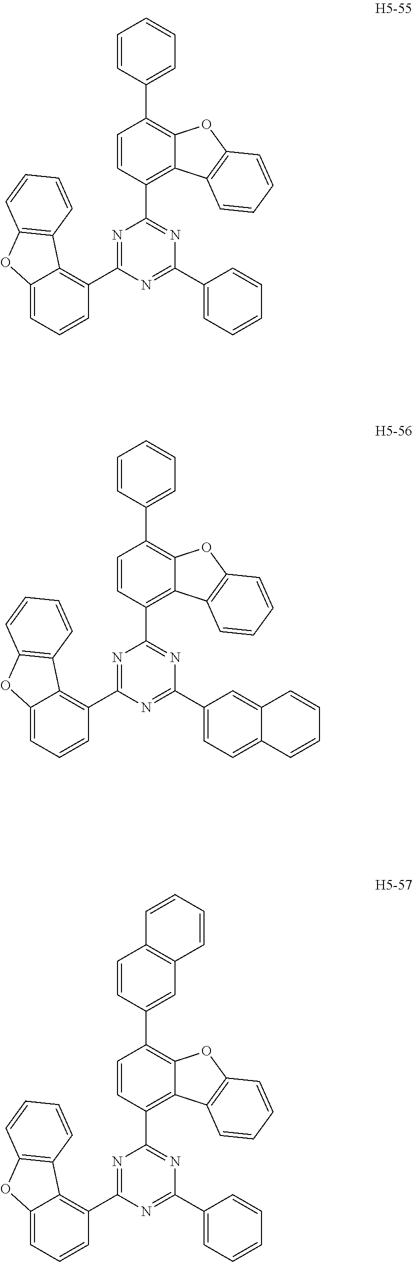 [SMILES: c1ccc(-c2nc(-c3cccc4oc5ccccc5c34)nc(-c3ccc(-c4ccccc4)c4oc5ccccc5c34)n2)cc1 ; c1ccc(-c2ccc(-c3nc(-c4ccc5ccccc5c4)nc(-c4cccc5oc6ccccc6c45)n3)c3c2oc2ccccc23)cc1 ; c1ccc(-c2nc(-c3cccc4oc5ccccc5c34)nc(-c3ccc(-c4ccc5ccccc5c4)c4oc5ccccc5c34)n2)cc1]

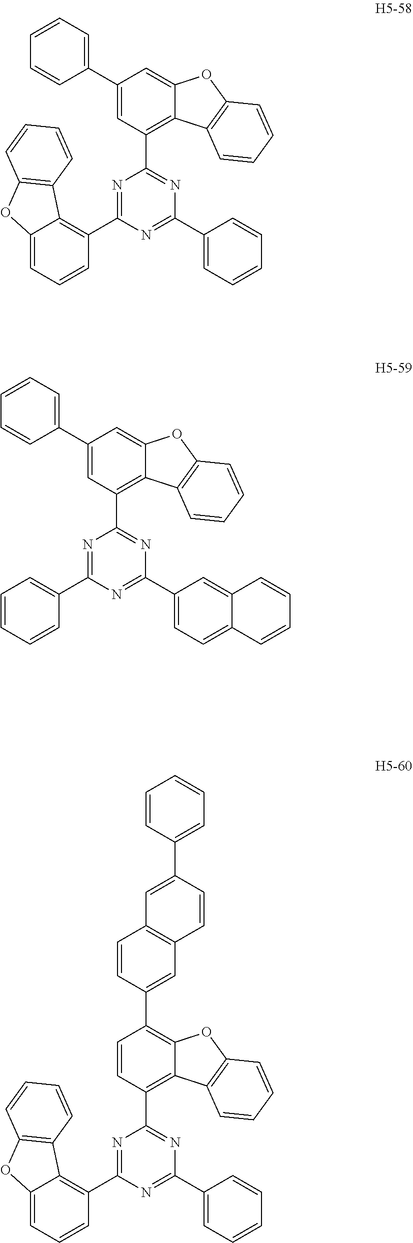 [SMILES: c1ccc(-c2cc(-c3nc(-c4ccccc4)nc(-c4cccc5oc6ccccc6c45)n3)c3c(c2)oc2ccccc23)cc1 ; c1ccc(-c2cc(-c3nc(-c4ccccc4)nc(-c4ccc5ccccc5c4)n3)c3c(c2)oc2ccccc23)cc1 ; c1ccc(-c2ccc3cc(-c4ccc(-c5nc(-c6ccccc6)nc(-c6cccc7oc8ccccc8c67)n5)c5c4oc4ccccc45)ccc3c2)cc1]

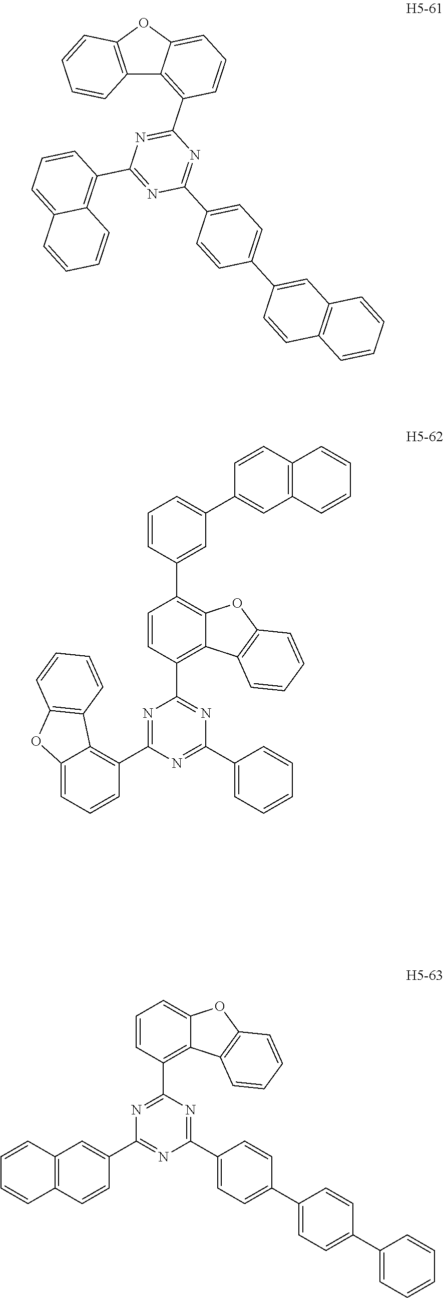 [SMILES: c1ccc2cc(-c3ccc(-c4nc(-c5cccc6ccccc56)nc(-c5cccc6oc7ccccc7c56)n4)cc3)ccc2c1 ; c1ccc(-c2nc(-c3cccc4oc5ccccc5c34)nc(-c3ccc(-c4cccc(-c5ccc6ccccc6c5)c4)c4oc5ccccc5c34)n2)cc1 ; c1ccc(-c2ccc(-c3ccc(-c4nc(-c5ccc6ccccc6c5)nc(-c5cccc6oc7ccccc7c56)n4)cc3)cc2)cc1]

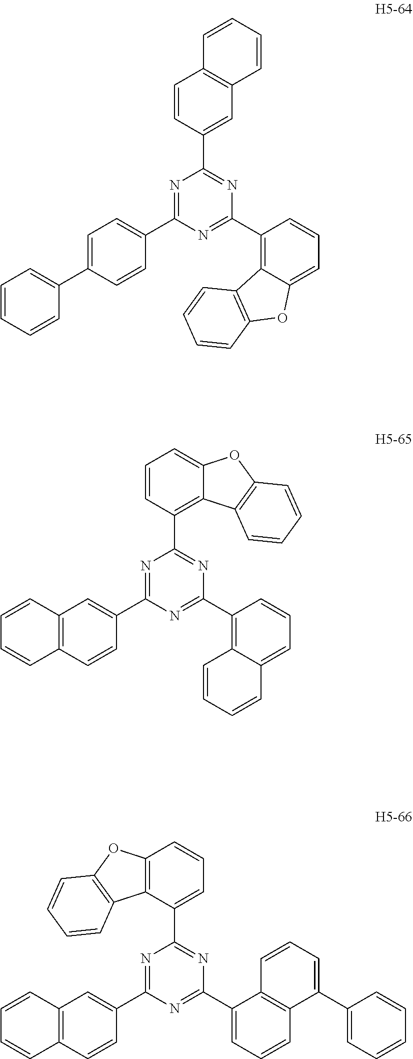 [SMILES: c1ccc(-c2ccc(-c3nc(-c4ccc5ccccc5c4)nc(-c4cccc5oc6ccccc6c45)n3)cc2)cc1 ; c1ccc2cc(-c3nc(-c4cccc5ccccc45)nc(-c4cccc5oc6ccccc6c45)n3)ccc2c1 ; c1ccc(-c2cccc3c(-c4nc(-c5ccc6ccccc6c5)nc(-c5cccc6oc7ccccc7c56)n4)cccc23)cc1]

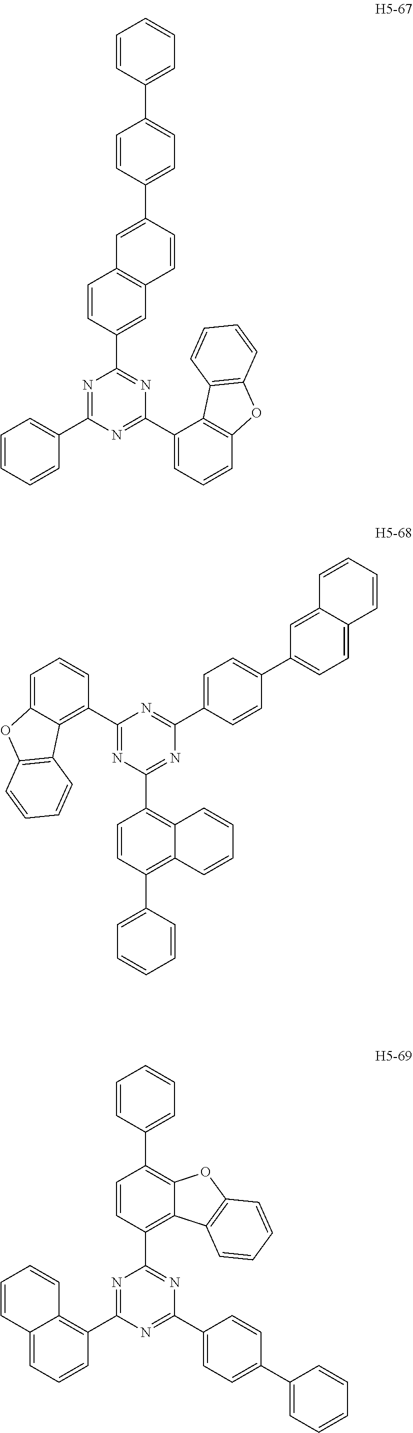 [SMILES: c1ccc(-c2ccc(-c3ccc4cc(-c5nc(-c6ccccc6)nc(-c6cccc7oc8ccccc8c67)n5)ccc4c3)cc2)cc1 ; c1ccc(-c2ccc(-c3nc(-c4ccc(-c5ccc6ccccc6c5)cc4)nc(-c4cccc5oc6ccccc6c45)n3)c3ccccc23)cc1 ; c1ccc(-c2ccc(-c3nc(-c4cccc5ccccc45)nc(-c4ccc(-c5ccccc5)c5oc6ccccc6c45)n3)cc2)cc1]

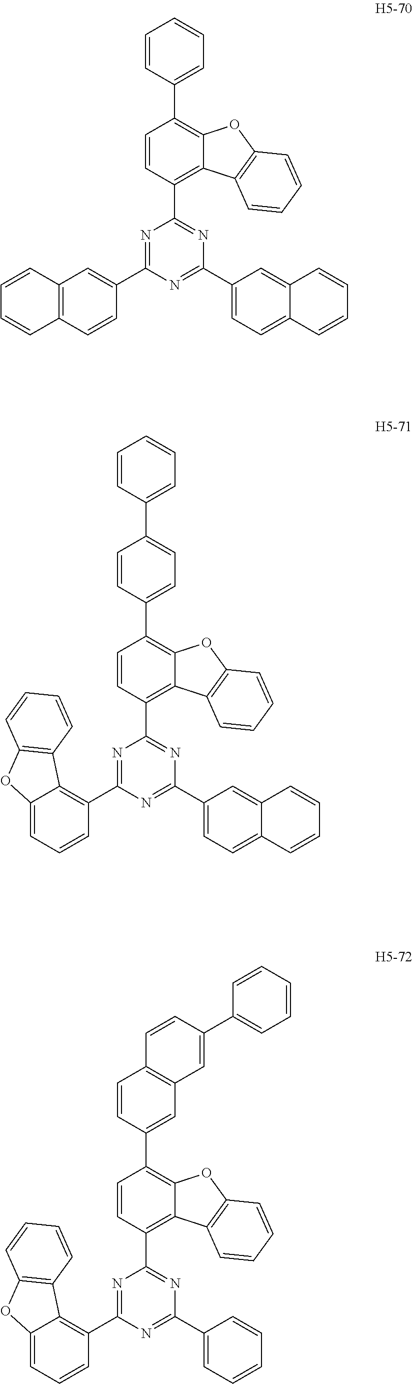 [SMILES: c1ccc(-c2ccc(-c3nc(-c4ccc5ccccc5c4)nc(-c4ccc5ccccc5c4)n3)c3c2oc2ccccc23)cc1 ; c1ccc(-c2ccc(-c3ccc(-c4nc(-c5ccc6ccccc6c5)nc(-c5cccc6oc7ccccc7c56)n4)c4c3oc3ccccc34)cc2)cc1 ; c1ccc(-c2ccc3ccc(-c4ccc(-c5nc(-c6ccccc6)nc(-c6cccc7oc8ccccc8c67)n5)c5c4oc4ccccc45)cc3c2)cc1]

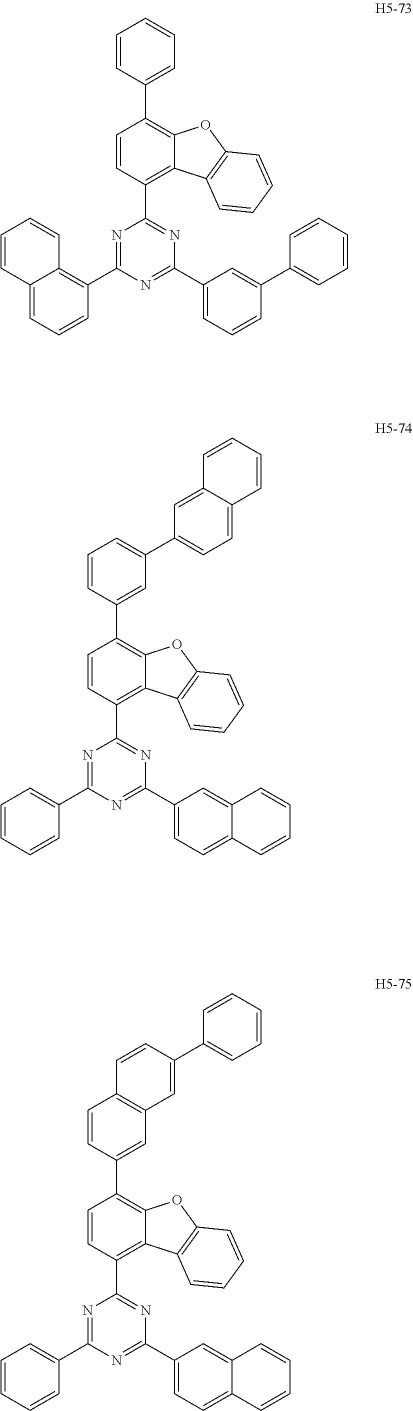 [SMILES: c1ccc(-c2cccc(-c3nc(-c4cccc5ccccc45)nc(-c4ccc(-c5ccccc5)c5oc6ccccc6c45)n3)c2)cc1 ; c1ccc(-c2nc(-c3ccc4ccccc4c3)nc(-c3ccc(-c4cccc(-c5ccc6ccccc6c5)c4)c4oc5ccccc5c34)n2)cc1 ; c1ccc(-c2ccc3ccc(-c4ccc(-c5nc(-c6ccccc6)nc(-c6ccc7ccccc7c6)n5)c5c4oc4ccccc45)cc3c2)cc1]

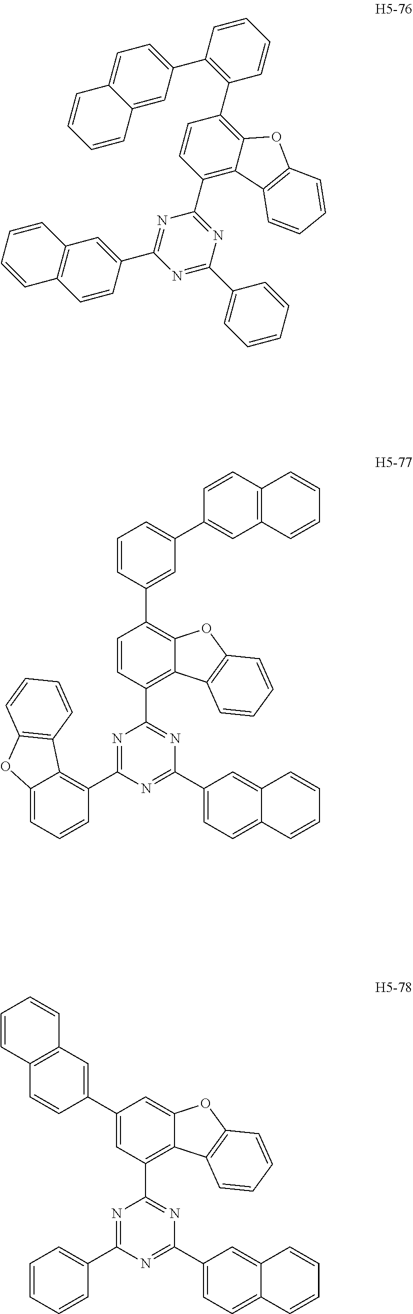 [SMILES: c1ccc(-c2nc(-c3ccc4ccccc4c3)nc(-c3ccc(-c4ccccc4-c4ccc5ccccc5c4)c4oc5ccccc5c34)n2)cc1 ; c1cc(-c2ccc3ccccc3c2)cc(-c2ccc(-c3nc(-c4ccc5ccccc5c4)nc(-c4cccc5oc6ccccc6c45)n3)c3c2oc2ccccc23)c1 ; c1ccc(-c2nc(-c3ccc4ccccc4c3)nc(-c3cc(-c4ccc5ccccc5c4)cc4oc5ccccc5c34)n2)cc1]

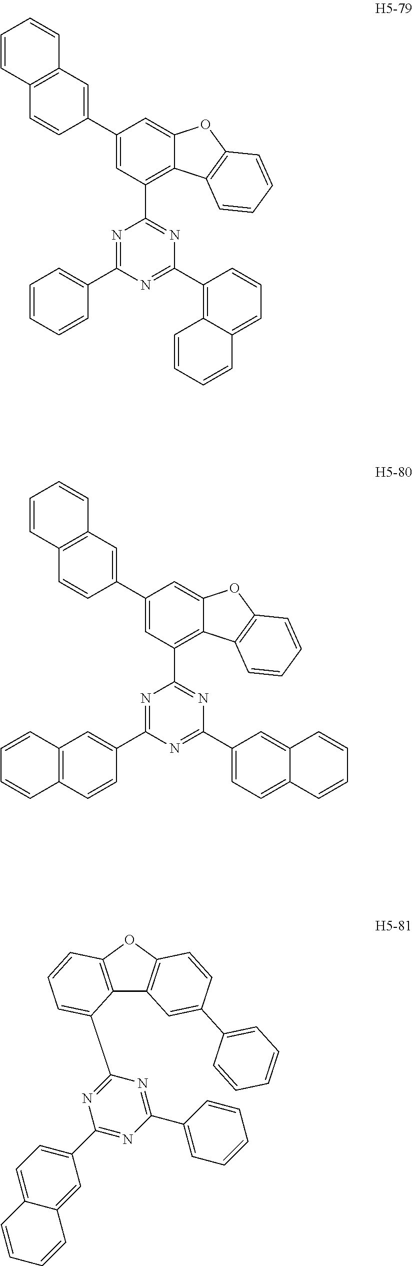 [SMILES: c1ccc(-c2nc(-c3cccc4ccccc34)nc(-c3cc(-c4ccc5ccccc5c4)cc4oc5ccccc5c34)n2)cc1 ; c1ccc2cc(-c3cc(-c4nc(-c5ccc6ccccc6c5)nc(-c5ccc6ccccc6c5)n4)c4c(c3)oc3ccccc34)ccc2c1 ; c1ccc(-c2ccc3oc4cccc(-c5nc(-c6ccccc6)nc(-c6ccc7ccccc7c6)n5)c4c3c2)cc1]

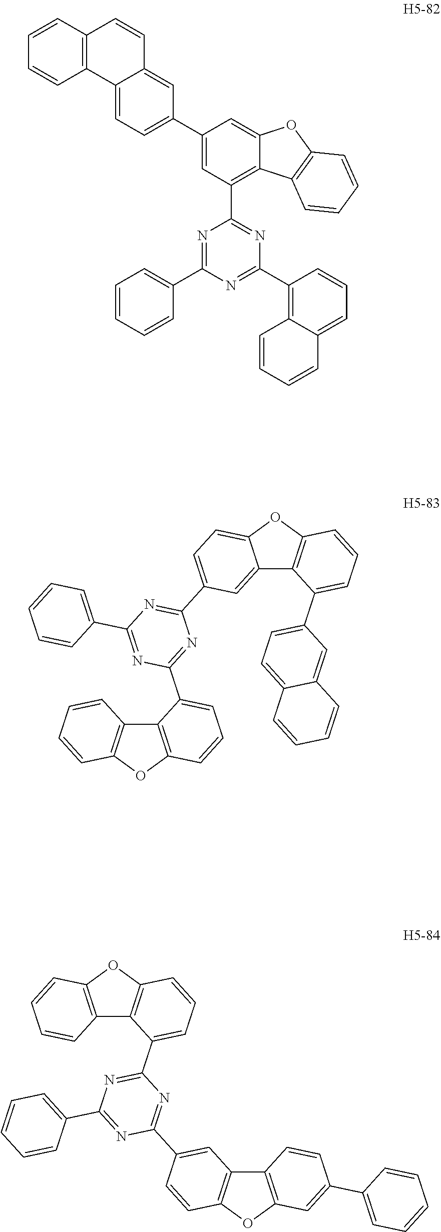 [SMILES: c1ccc(-c2nc(-c3cccc4ccccc34)nc(-c3cc(-c4ccc5c(ccc6ccccc65)c4)cc4oc5ccccc5c34)n2)cc1 ; c1ccc(-c2nc(-c3ccc4oc5cccc(-c6ccc7ccccc7c6)c5c4c3)nc(-c3cccc4oc5ccccc5c34)n2)cc1 ; c1ccc(-c2ccc3c(c2)oc2ccc(-c4nc(-c5ccccc5)nc(-c5cccc6oc7ccccc7c56)n4)cc23)cc1]

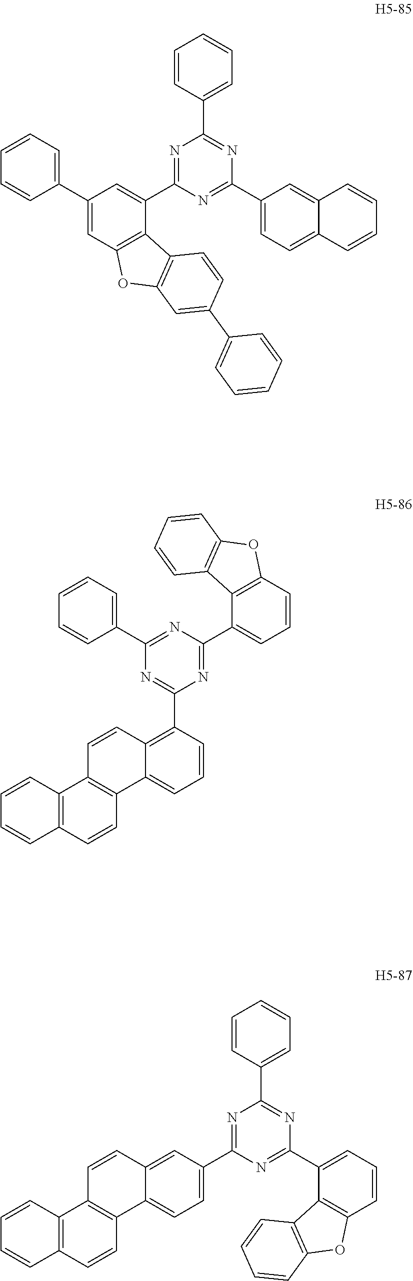 [SMILES: c1ccc(-c2ccc3c(c2)oc2cc(-c4ccccc4)cc(-c4nc(-c5ccccc5)nc(-c5ccc6ccccc6c5)n4)c23)cc1 ; c1ccc(-c2nc(-c3cccc4c3ccc3c5ccccc5ccc43)nc(-c3cccc4oc5ccccc5c34)n2)cc1 ; c1ccc(-c2nc(-c3ccc4c(ccc5c6ccccc6ccc45)c3)nc(-c3cccc4oc5ccccc5c34)n2)cc1]

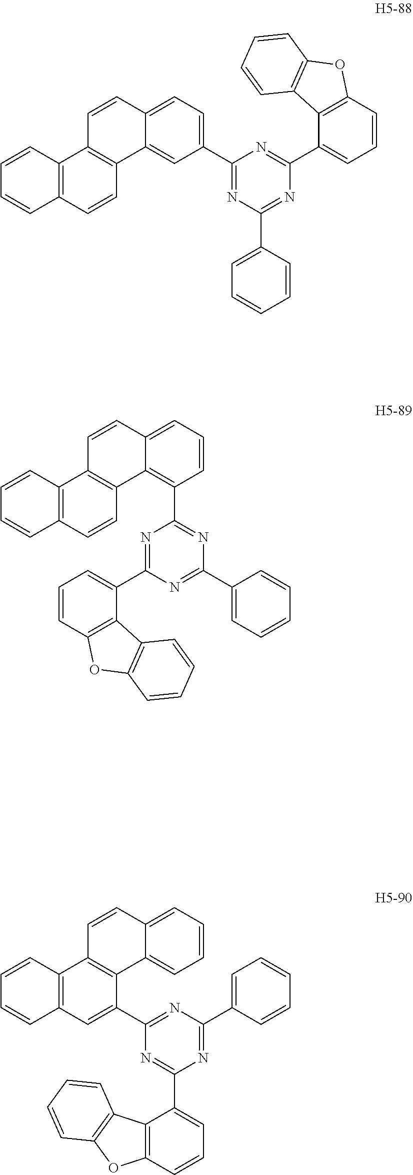 [SMILES: c1ccc(-c2nc(-c3ccc4ccc5c6ccccc6ccc5c4c3)nc(-c3cccc4oc5ccccc5c34)n2)cc1 ; c1ccc(-c2nc(-c3cccc4ccc5c6ccccc6ccc5c34)nc(-c3cccc4oc5ccccc5c34)n2)cc1 ; c1ccc(-c2nc(-c3cccc4oc5ccccc5c34)nc(-c3cc4ccccc4c4ccc5ccccc5c34)n2)cc1]

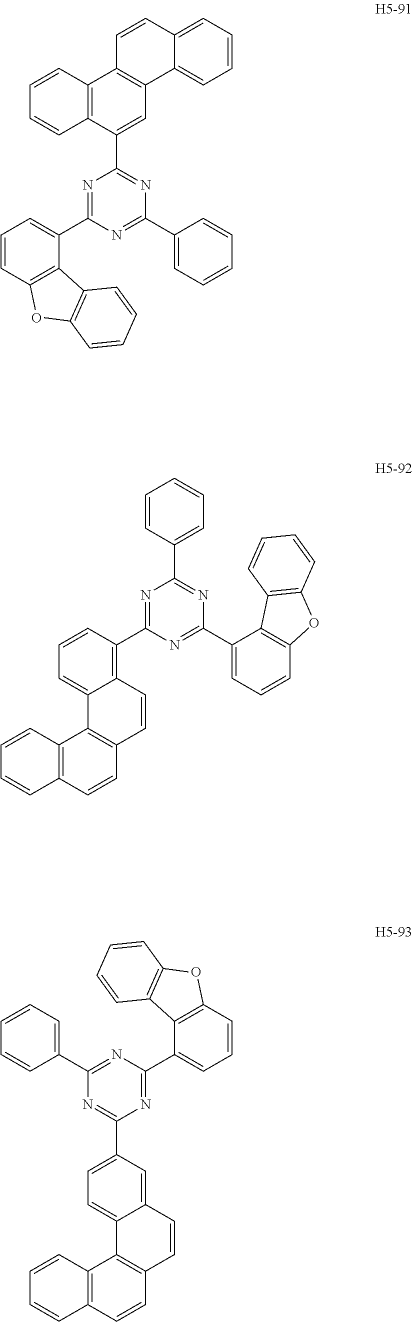 [SMILES: c1ccc(-c2nc(-c3cc4c5ccccc5ccc4c4ccccc34)nc(-c3cccc4oc5ccccc5c34)n2)cc1 ; c1ccc(-c2nc(-c3cccc4c3ccc3ccc5ccccc5c34)nc(-c3cccc4oc5ccccc5c34)n2)cc1 ; c1ccc(-c2nc(-c3ccc4c(ccc5ccc6ccccc6c54)c3)nc(-c3cccc4oc5ccccc5c34)n2)cc1]

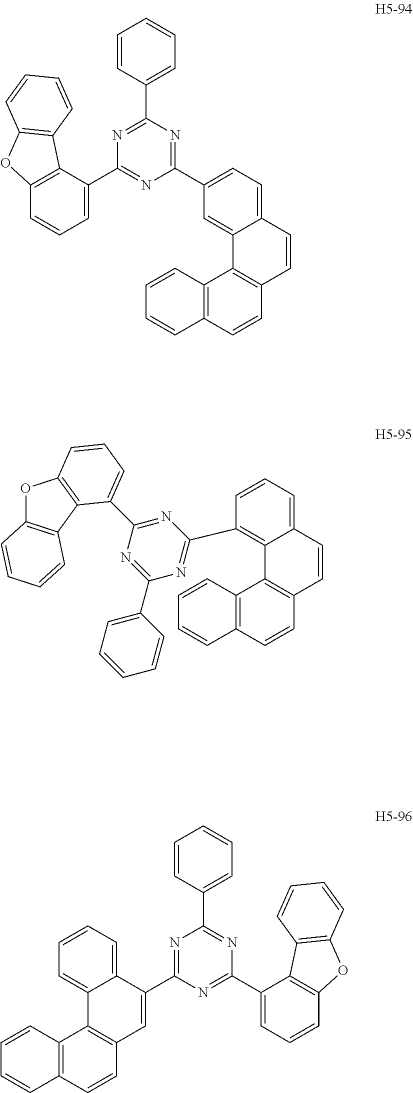 [SMILES: c1ccc(-c2nc(-c3ccc4ccc5ccc6ccccc6c5c4c3)nc(-c3cccc4oc5ccccc5c34)n2)cc1 ; c1ccc(-c2nc(-c3cccc4oc5ccccc5c34)nc(-c3cccc4ccc5ccc6ccccc6c5c34)n2)cc1 ; c1ccc(-c2nc(-c3cc4ccc5ccccc5c4c4ccccc34)nc(-c3cccc4oc5ccccc5c34)n2)cc1]

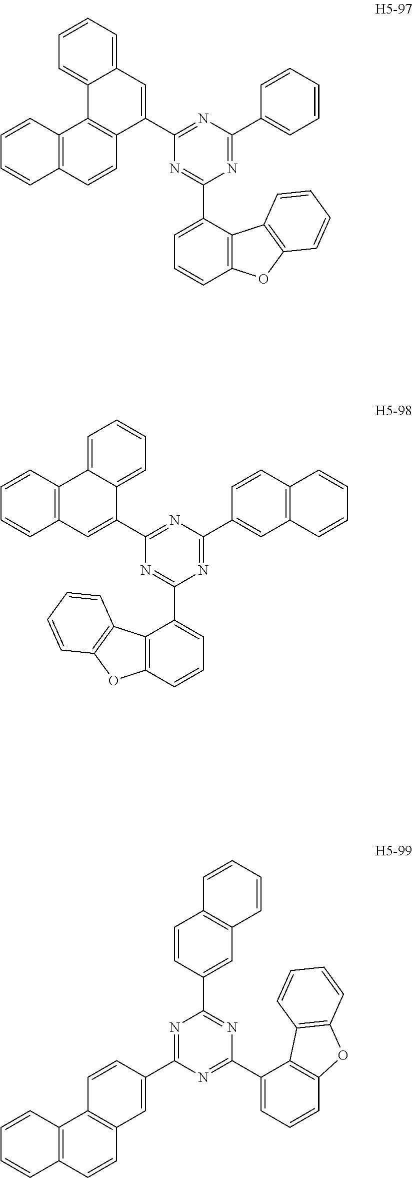 [SMILES: c1ccc(-c2nc(-c3cc4ccccc4c4c3ccc3ccccc34)nc(-c3cccc4oc5ccccc5c34)n2)cc1 ; c1ccc2cc(-c3nc(-c4cc5ccccc5c5ccccc45)nc(-c4cccc5oc6ccccc6c45)n3)ccc2c1 ; c1ccc2cc(-c3nc(-c4ccc5c(ccc6ccccc65)c4)nc(-c4cccc5oc6ccccc6c45)n3)ccc2c1]

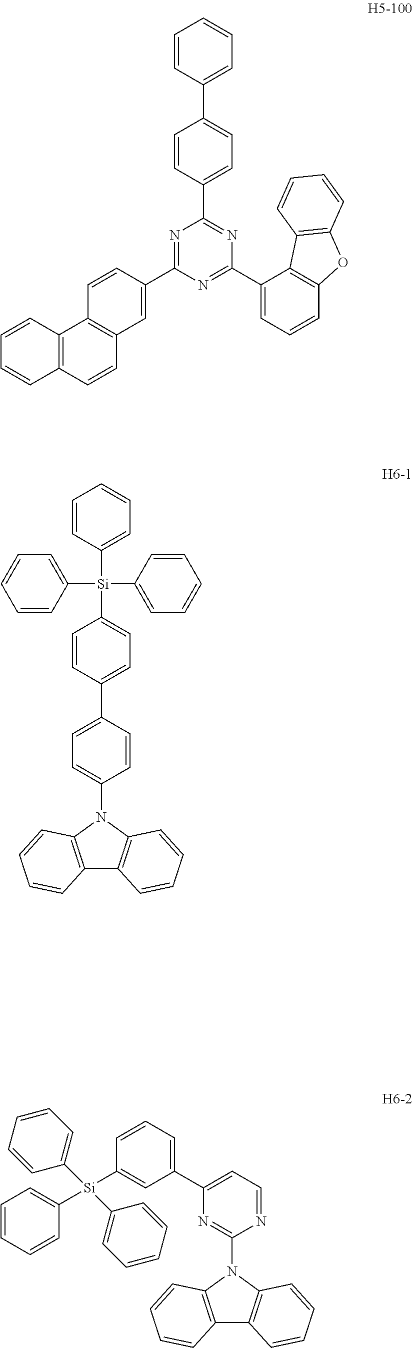 [SMILES: c1ccc(-c2ccc(-c3nc(-c4ccc5c(ccc6ccccc65)c4)nc(-c4cccc5oc6ccccc6c45)n3)cc2)cc1 ; c1ccc([Si](c2ccccc2)(c2ccccc2)c2ccc(-c3ccc(-n4c5ccccc5c5ccccc54)cc3)cc2)cc1 ; c1ccc([Si](c2ccccc2)(c2ccccc2)c2cccc(-c3ccnc(-n4c5ccccc5c5ccccc54)n3)c2)cc1]

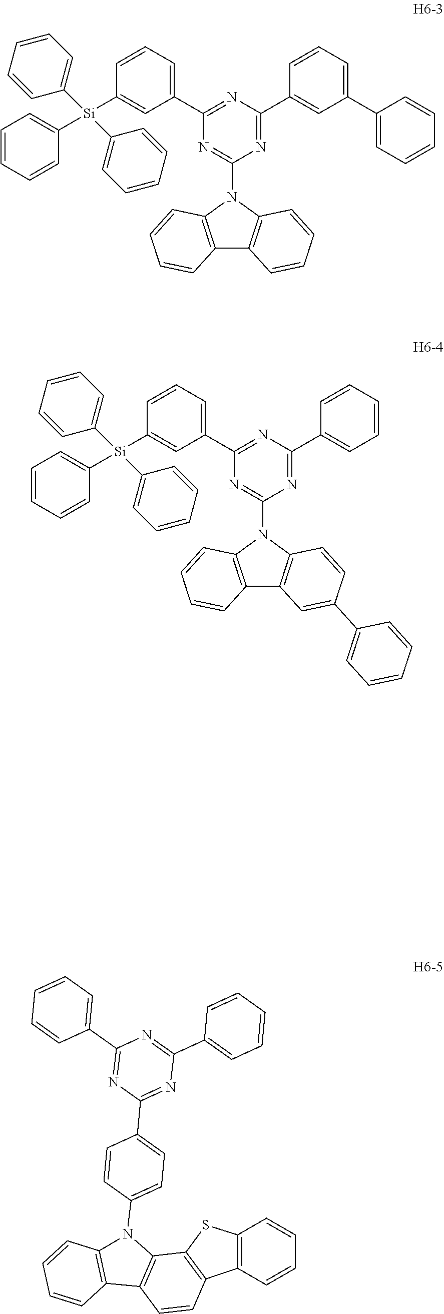 [SMILES: c1ccc(-c2cccc(-c3nc(-c4cccc([Si](c5ccccc5)(c5ccccc5)c5ccccc5)c4)nc(-n4c5ccccc5c5ccccc54)n3)c2)cc1 ; c1ccc(-c2ccc3c(c2)c2ccccc2n3-c2nc(-c3ccccc3)nc(-c3cccc([Si](c4ccccc4)(c4ccccc4)c4ccccc4)c3)n2)cc1 ; c1ccc(-c2nc(-c3ccccc3)nc(-c3ccc(-n4c5ccccc5c5ccc6c7ccccc7sc6c54)cc3)n2)cc1]

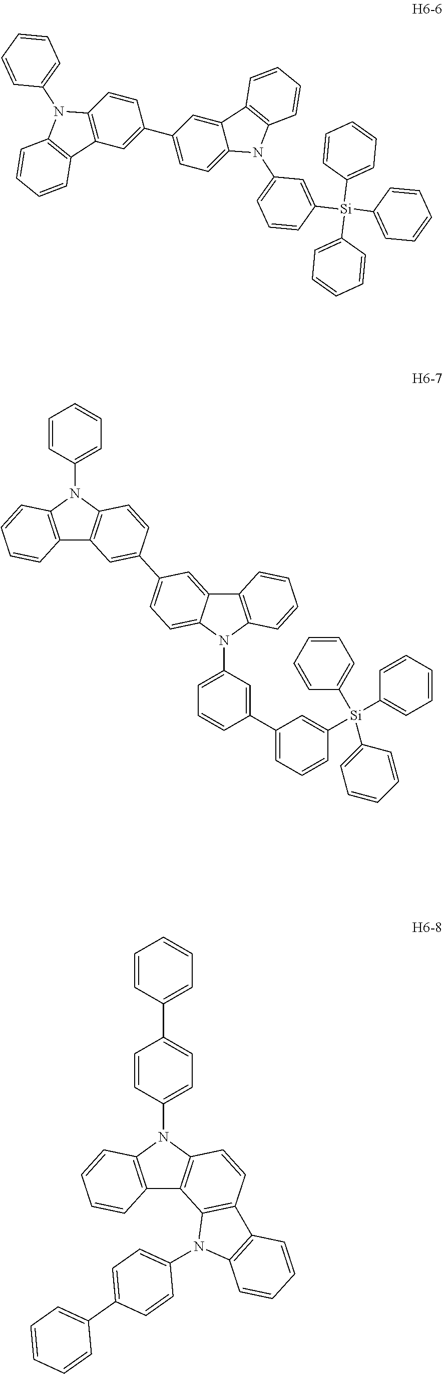 [SMILES: c1ccc(-n2c3ccccc3c3cc(-c4ccc5c(c4)c4ccccc4n5-c4cccc([Si](c5ccccc5)(c5ccccc5)c5ccccc5)c4)ccc32)cc1 ; c1ccc(-n2c3ccccc3c3cc(-c4ccc5c(c4)c4ccccc4n5-c4cccc(-c5cccc([Si](c6ccccc6)(c6ccccc6)c6ccccc6)c5)c4)ccc32)cc1 ; c1ccc(-c2ccc(-n3c4ccccc4c4c3ccc3c5ccccc5n(-c5ccc(-c6ccccc6)cc5)c34)cc2)cc1]

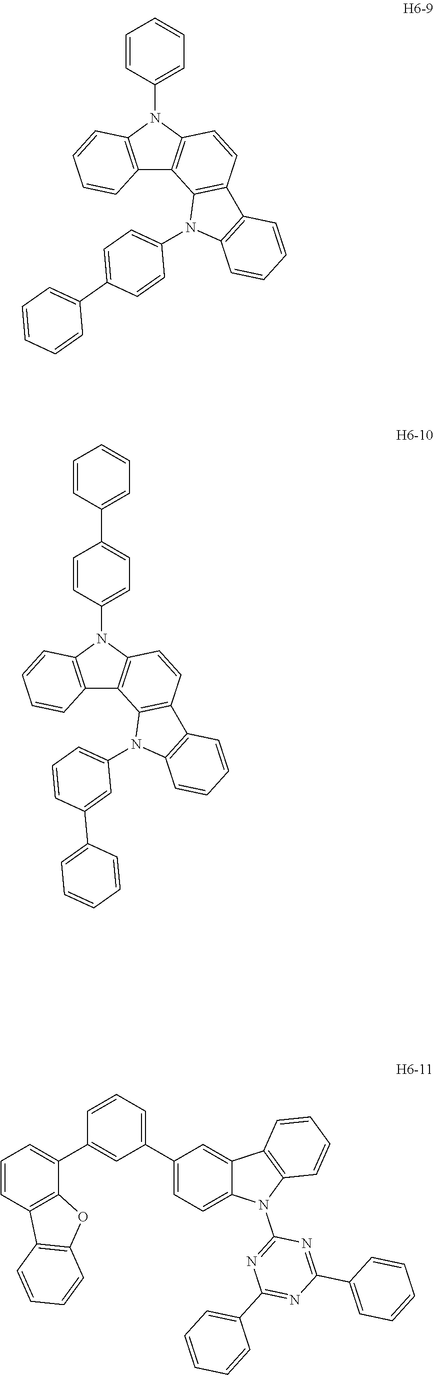 [SMILES: c1ccc(-c2ccc(-n3c4ccccc4c4ccc5c(c6ccccc6n5-c5ccccc5)c43)cc2)cc1 ; c1ccc(-c2ccc(-n3c4ccccc4c4c3ccc3c5ccccc5n(-c5cccc(-c6ccccc6)c5)c34)cc2)cc1 ; c1ccc(-c2nc(-c3ccccc3)nc(-n3c4ccccc4c4cc(-c5cccc(-c6cccc7c6oc6ccccc67)c5)ccc43)n2)cc1]

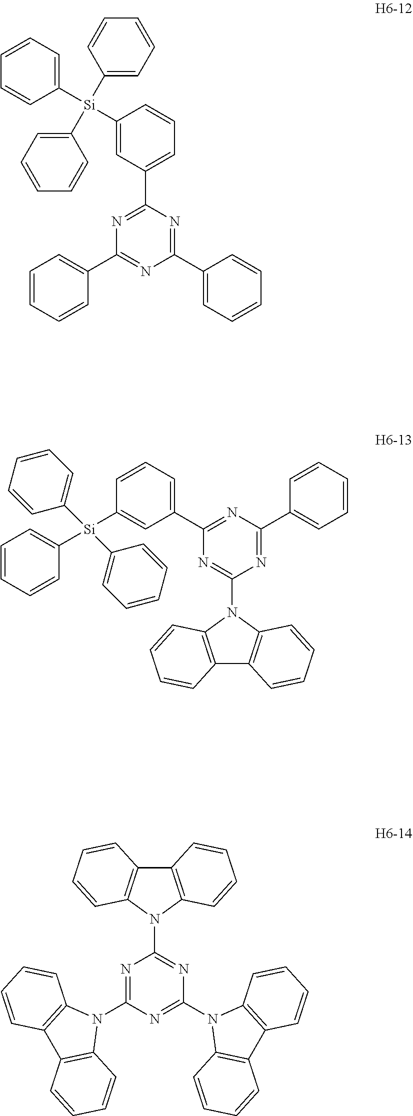 [SMILES: c1ccc(-c2nc(-c3ccccc3)nc(-c3cccc([Si](c4ccccc4)(c4ccccc4)c4ccccc4)c3)n2)cc1 ; c1ccc(-c2nc(-c3cccc([Si](c4ccccc4)(c4ccccc4)c4ccccc4)c3)nc(-n3c4ccccc4c4ccccc43)n2)cc1 ; c1ccc2c(c1)c1ccccc1n2-c1nc(-n2c3ccccc3c3ccccc32)nc(-n2c3ccccc3c3ccccc32)n1]

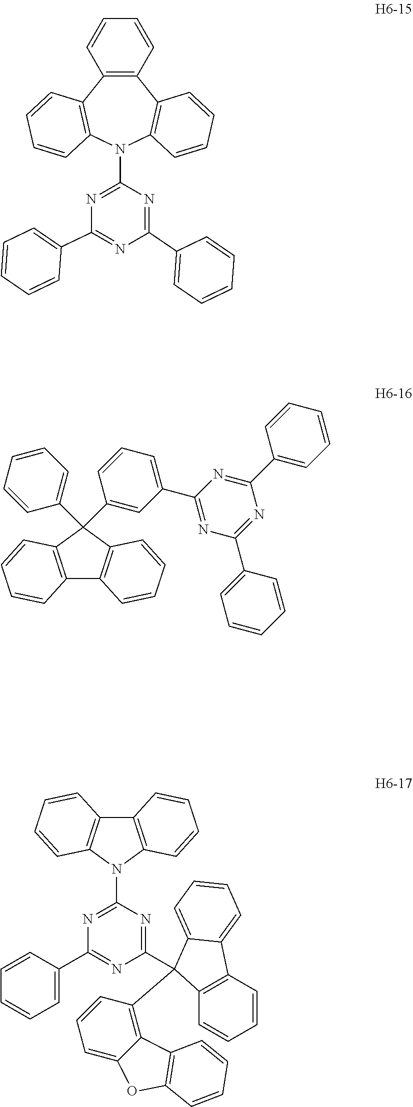 [SMILES: c1ccc(-c2nc(-c3ccccc3)nc(N3c4ccccc4-c4ccccc4-c4ccccc43)n2)cc1 ; c1ccc(-c2nc(-c3ccccc3)nc(-c3cccc(C4(c5ccccc5)c5ccccc5-c5ccccc54)c3)n2)cc1 ; c1ccc(-c2nc(-n3c4ccccc4c4ccccc43)nc(C3(c4cccc5oc6ccccc6c45)c4ccccc4-c4ccccc43)n2)cc1]

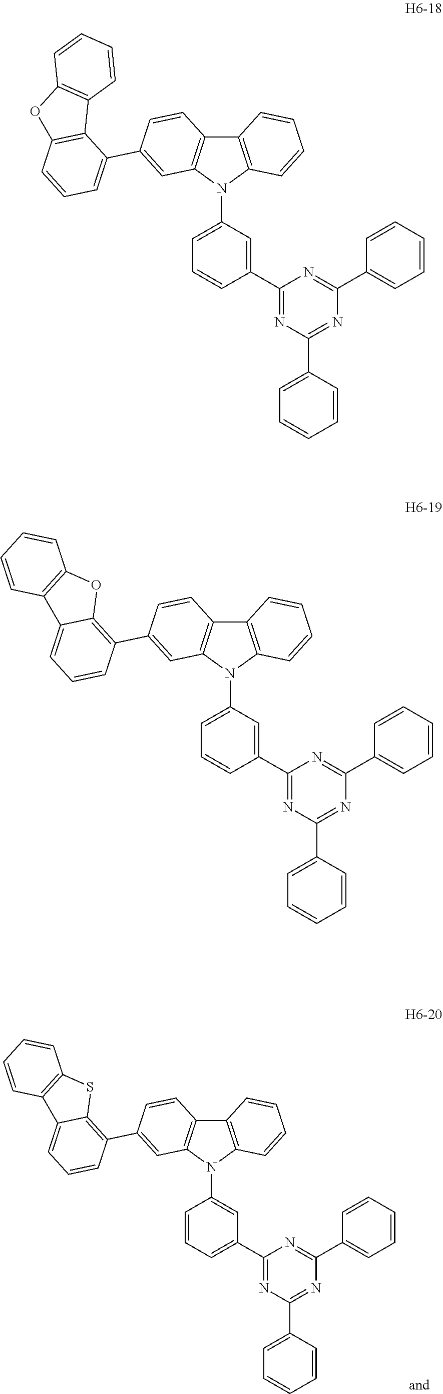 [SMILES: c1ccc(-c2nc(-c3ccccc3)nc(-c3cccc(-n4c5ccccc5c5ccc(-c6cccc7oc8ccccc8c67)cc54)c3)n2)cc1 ; c1ccc(-c2nc(-c3ccccc3)nc(-c3cccc(-n4c5ccccc5c5ccc(-c6cccc7c6oc6ccccc67)cc54)c3)n2)cc1 ; c1ccc(-c2nc(-c3ccccc3)nc(-c3cccc(-n4c5ccccc5c5ccc(-c6cccc7c6sc6ccccc67)cc54)c3)n2)cc1]

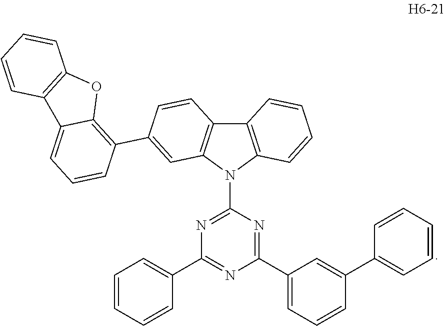 [SMILES: c1ccc(-c2cccc(-c3nc(-c4ccccc4)nc(-n4c5ccccc5c5ccc(-c6cccc7c6oc6ccccc67)cc54)n3)c2)cc1]

11. The organic electroluminescent device according to claim 8, wherein the compound represented by Formula 2 is selected from the following compounds: 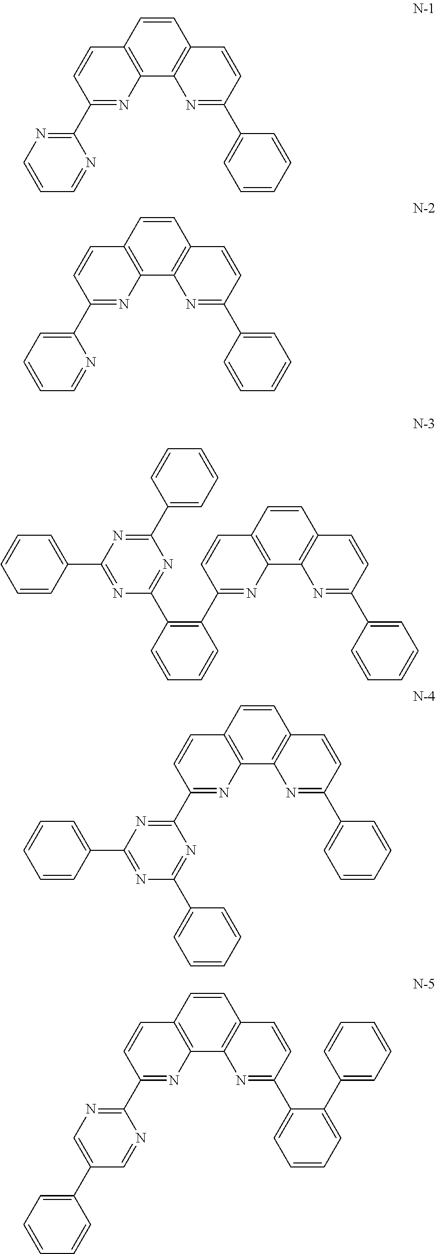 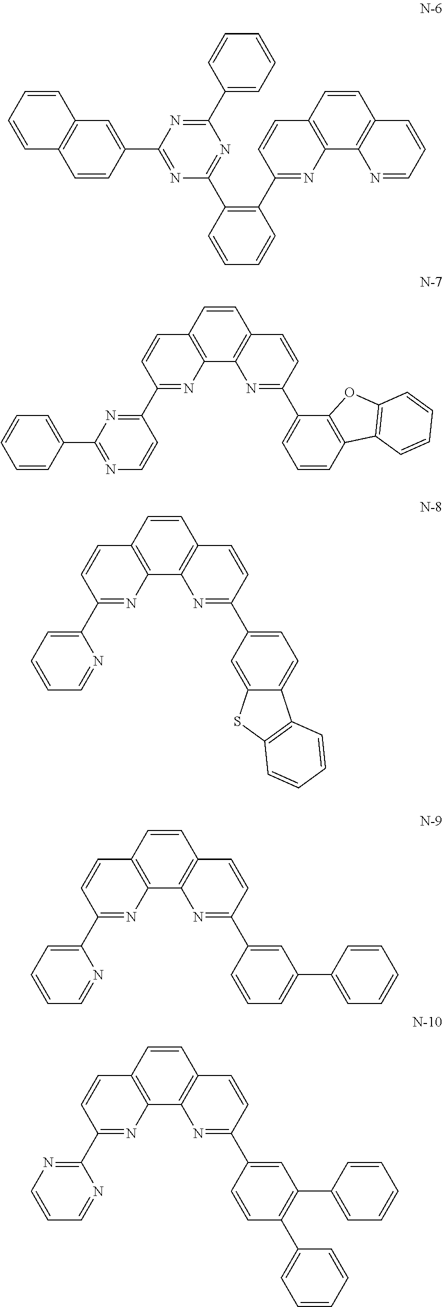 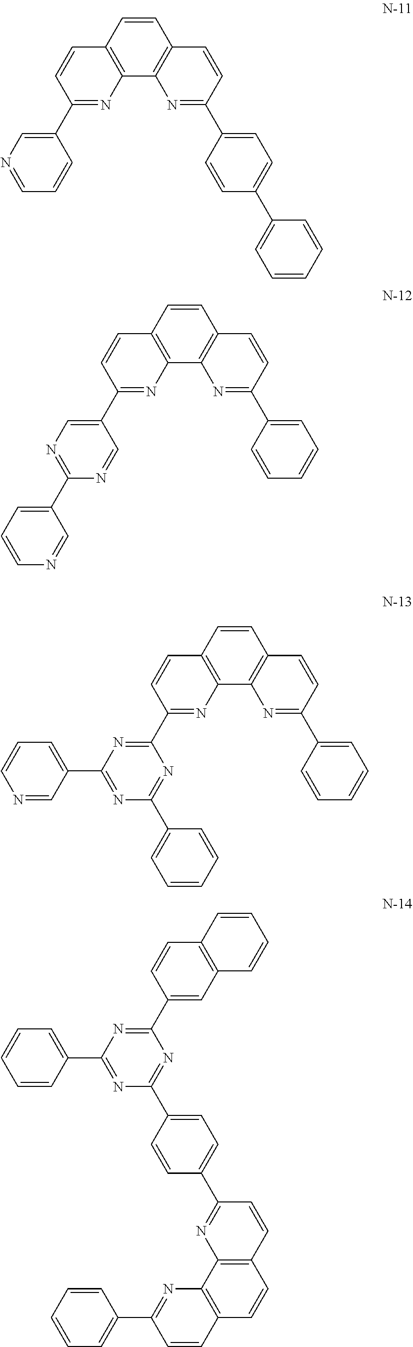 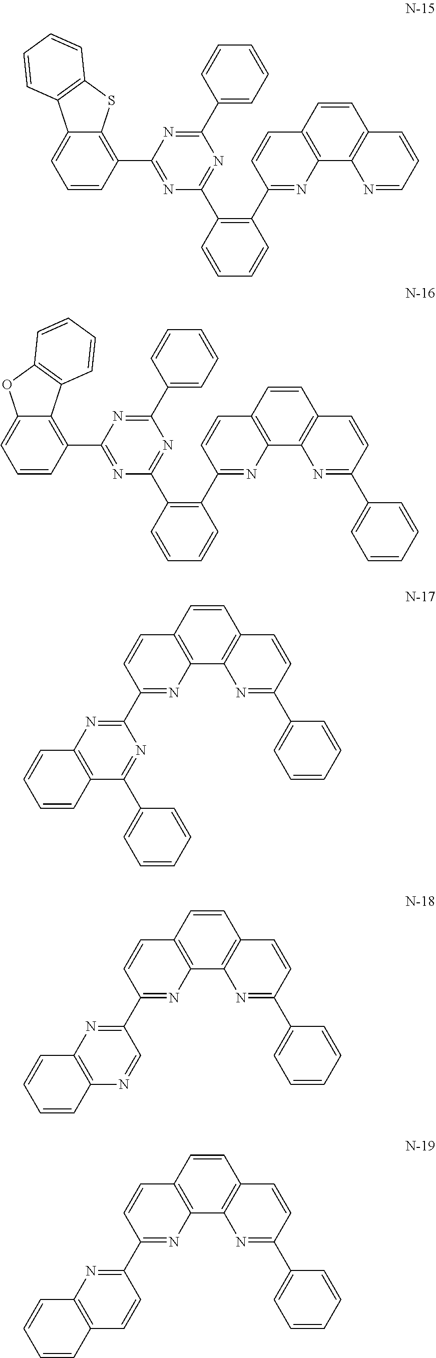 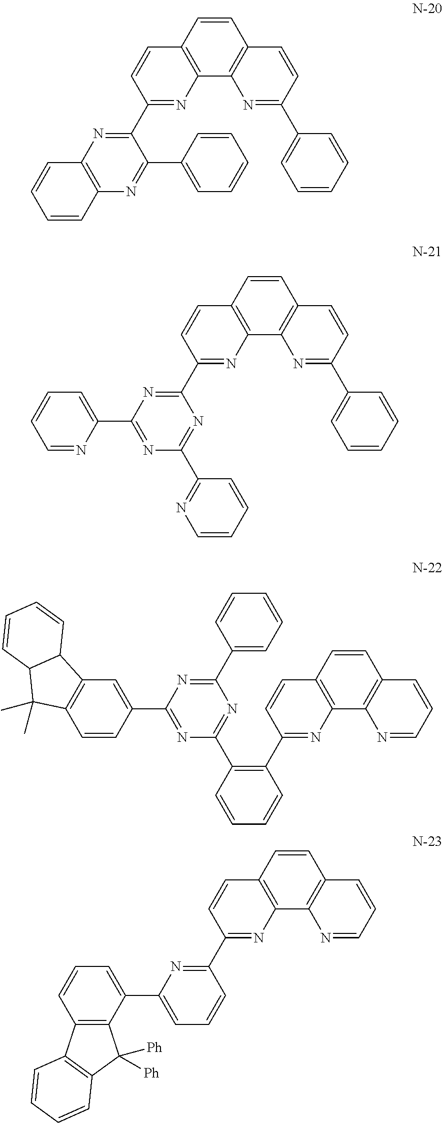 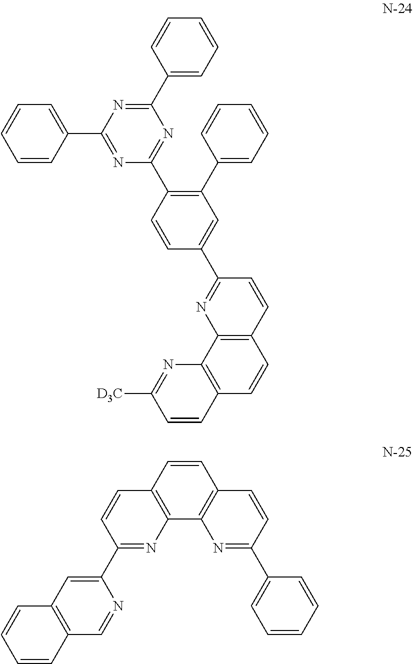 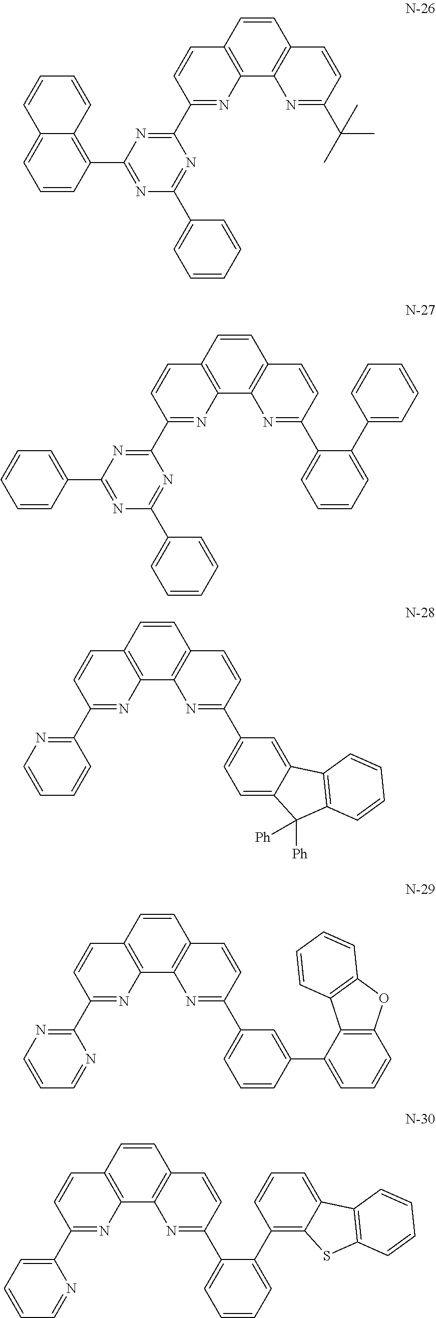 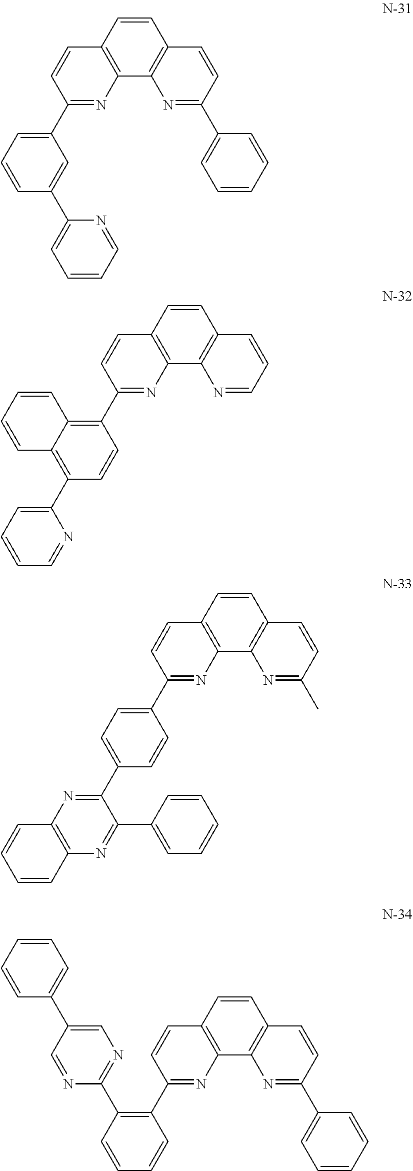 [SMILES: c1ccc(-c2ccc3ccc4ccc(-c5cccc(-c6ccccn6)c5)nc4c3n2)cc1 ; c1ccc(-c2ccc(-c3ccc4ccc5cccnc5c4n3)c3ccccc23)nc1 ; Cc1ccc2ccc3ccc(-c4ccc(-c5nc6ccccc6nc5-c5ccccc5)cc4)nc3c2n1 ; c1ccc(-c2cnc(-c3ccccc3-c3ccc4ccc5ccc(-c6ccccc6)nc5c4n3)nc2)cc1] 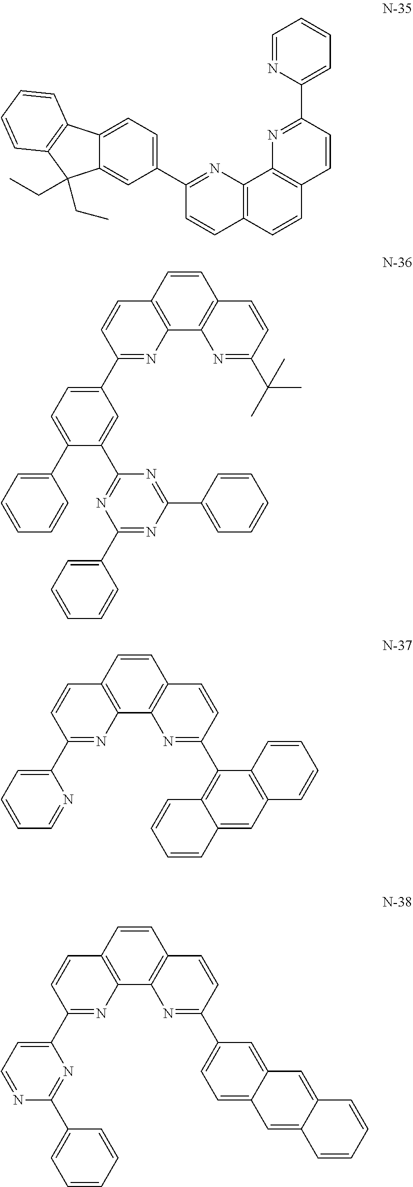 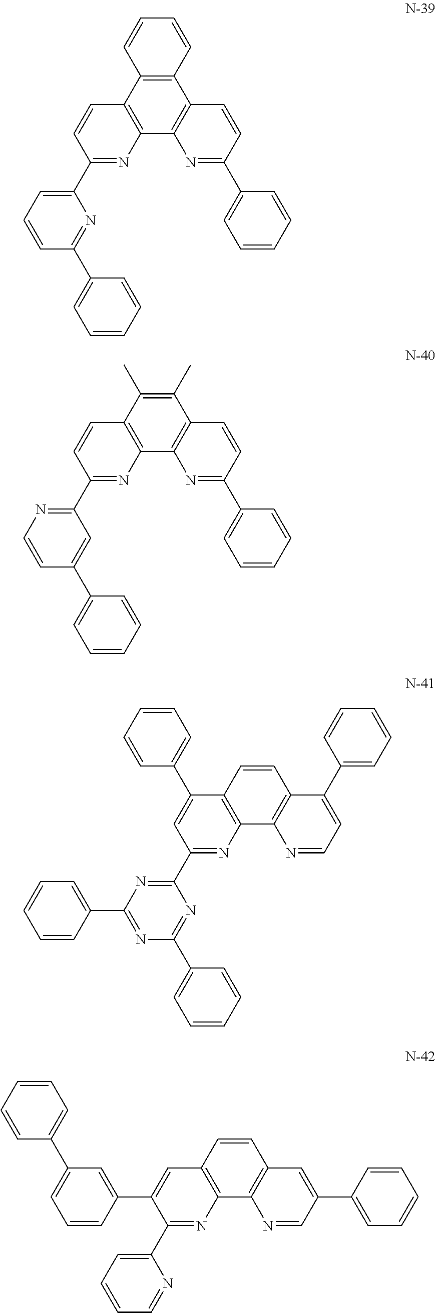 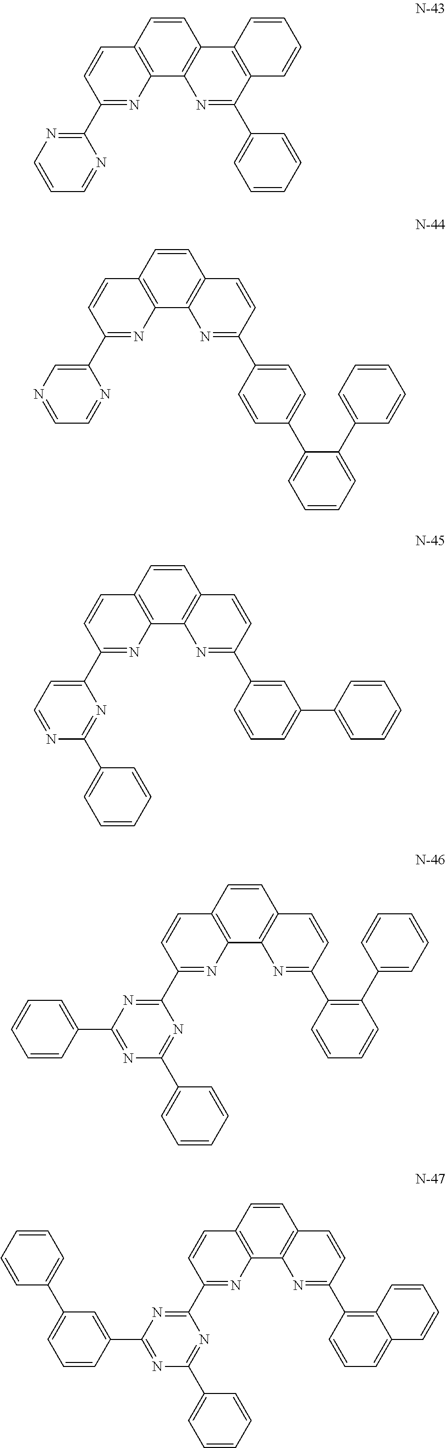 [SMILES: c1ccc(-c2nc3c(ccc4ccc(-c5ncccn5)nc43)c3ccccc23)cc1 ; c1ccc(-c2ccccc2-c2ccc(-c3ccc4ccc5ccc(-c6cnccn6)nc5c4n3)cc2)cc1 ; c1ccc(-c2cccc(-c3ccc4ccc5ccc(-c6ccnc(-c7ccccc7)n6)nc5c4n3)c2)cc1 ; c1ccc(-c2nc(-c3ccccc3)nc(-c3ccc4ccc5ccc(-c6ccccc6-c6ccccc6)nc5c4n3)n2)cc1 ; c1ccc(-c2cccc(-c3nc(-c4ccccc4)nc(-c4ccc5ccc6ccc(-c7cccc8ccccc78)nc6c5n4)n3)c2)cc1] 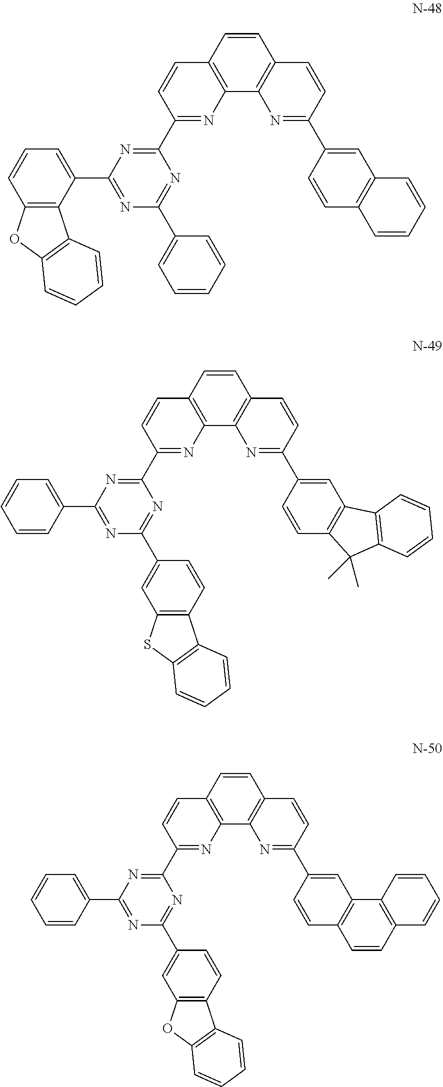 [SMILES: c1ccc(-c2nc(-c3ccc4ccc5ccc(-c6ccc7ccccc7c6)nc5c4n3)nc(-c3cccc4oc5ccccc5c34)n2)cc1 ; CC1(C)c2ccccc2-c2cc(-c3ccc4ccc5ccc(-c6nc(-c7ccccc7)nc(-c7ccc8c(c7)sc7ccccc78)n6)nc5c4n3)ccc21 ; c1ccc(-c2nc(-c3ccc4c(c3)oc3ccccc34)nc(-c3ccc4ccc5ccc(-c6ccc7ccc8ccccc8c7c6)nc5c4n3)n2)cc1] 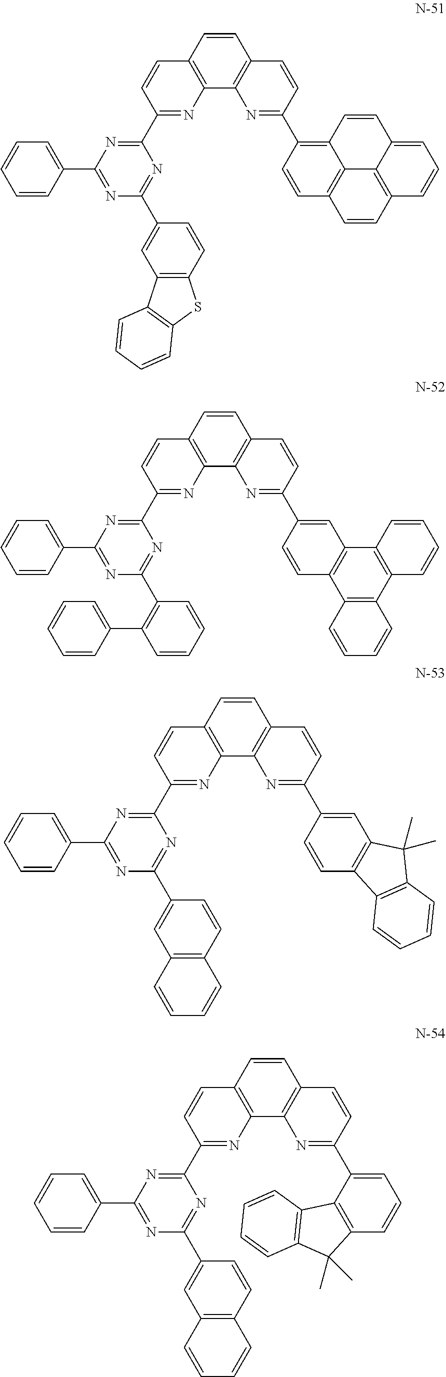 [SMILES: c1ccc(-c2nc(-c3ccc4sc5ccccc5c4c3)nc(-c3ccc4ccc5ccc(-c6ccc7ccc8cccc9ccc6c7c89)nc5c4n3)n2)cc1 ; c1ccc(-c2nc(-c3ccc4ccc5ccc(-c6ccc7c8ccccc8c8ccccc8c7c6)nc5c4n3)nc(-c3ccccc3-c3ccccc3)n2)cc1 ; CC1(C)c2ccccc2-c2ccc(-c3ccc4ccc5ccc(-c6nc(-c7ccccc7)nc(-c7ccc8ccccc8c7)n6)nc5c4n3)cc21 ; CC1(C)c2ccccc2-c2c(-c3ccc4ccc5ccc(-c6nc(-c7ccccc7)nc(-c7ccc8ccccc8c7)n6)nc5c4n3)cccc21] 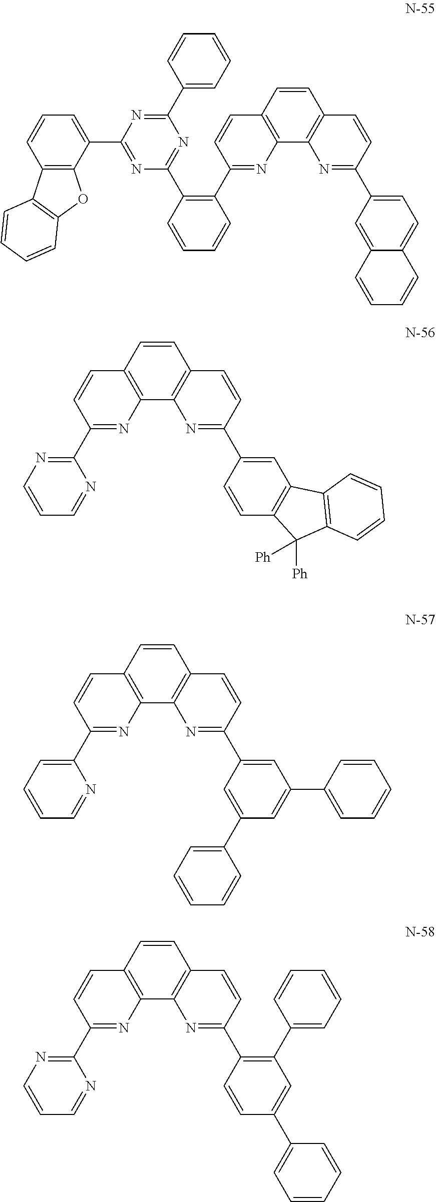 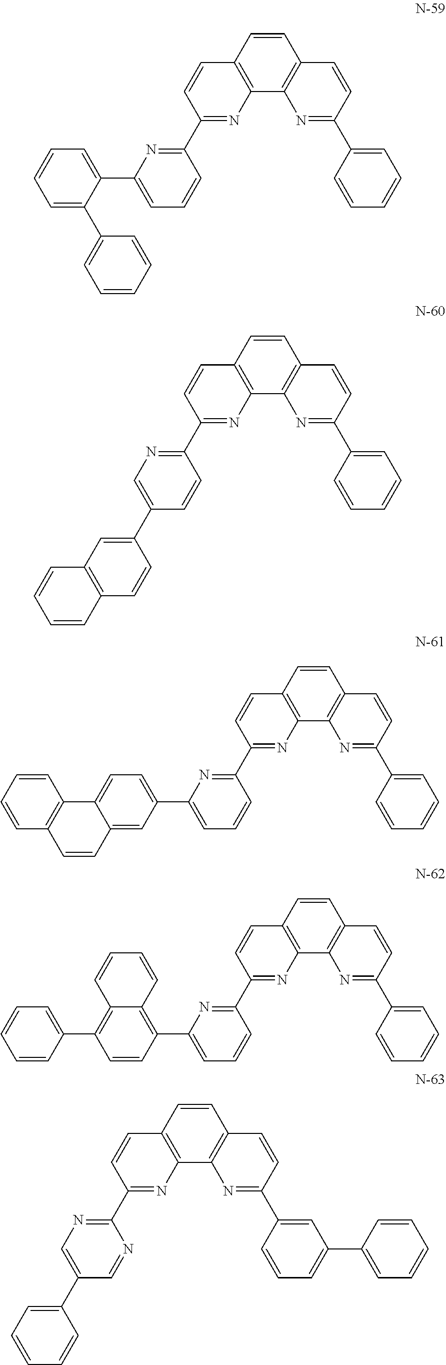 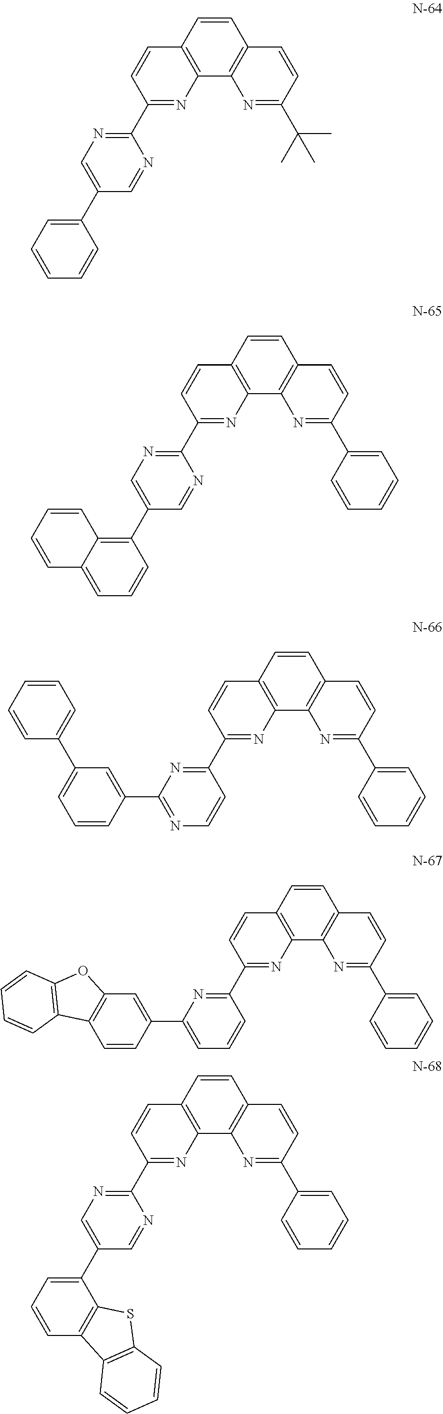 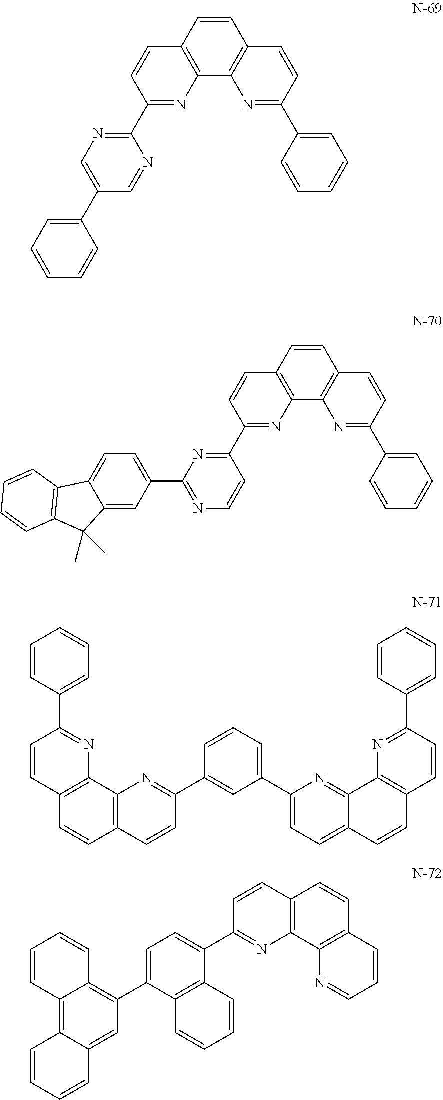 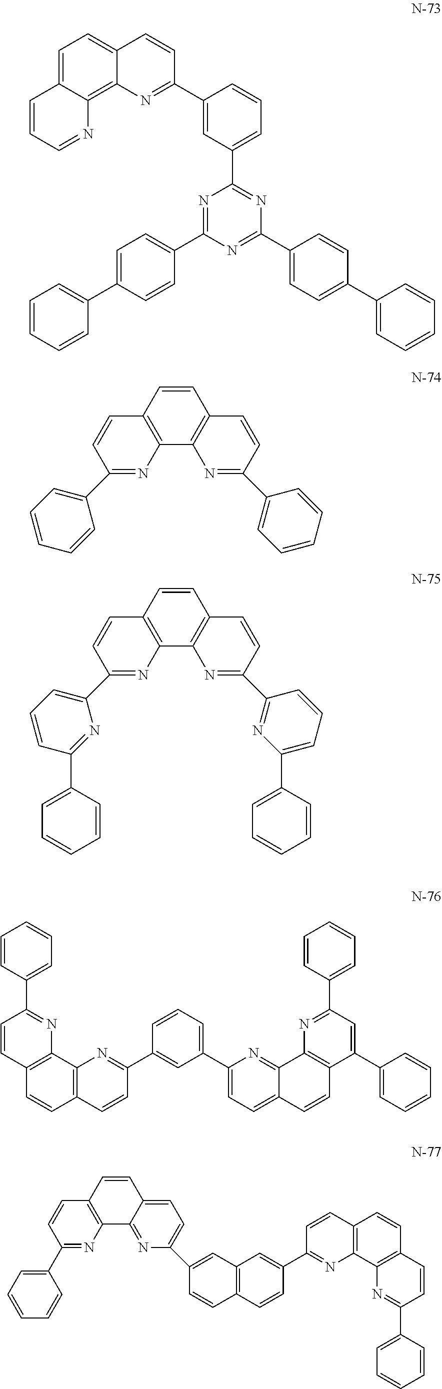 [SMILES: c1ccc(-c2ccc(-c3nc(-c4ccc(-c5ccccc5)cc4)nc(-c4cccc(-c5ccc6ccc7cccnc7c6n5)c4)n3)cc2)cc1 ; c1ccc(-c2ccc3ccc4ccc(-c5ccccc5)nc4c3n2)cc1 ; c1ccc(-c2cccc(-c3ccc4ccc5ccc(-c6cccc(-c7ccccc7)n6)nc5c4n3)n2)cc1 ; c1ccc(-c2ccc3ccc4ccc(-c5cccc(-c6ccc7ccc8c(-c9ccccc9)cc(-c9ccccc9)nc8c7n6)c5)nc4c3n2)cc1 ; c1ccc(-c2ccc3ccc4ccc(-c5ccc6ccc(-c7ccc8ccc9ccc(-c%10ccccc%10)nc9c8n7)cc6c5)nc4c3n2)cc1] 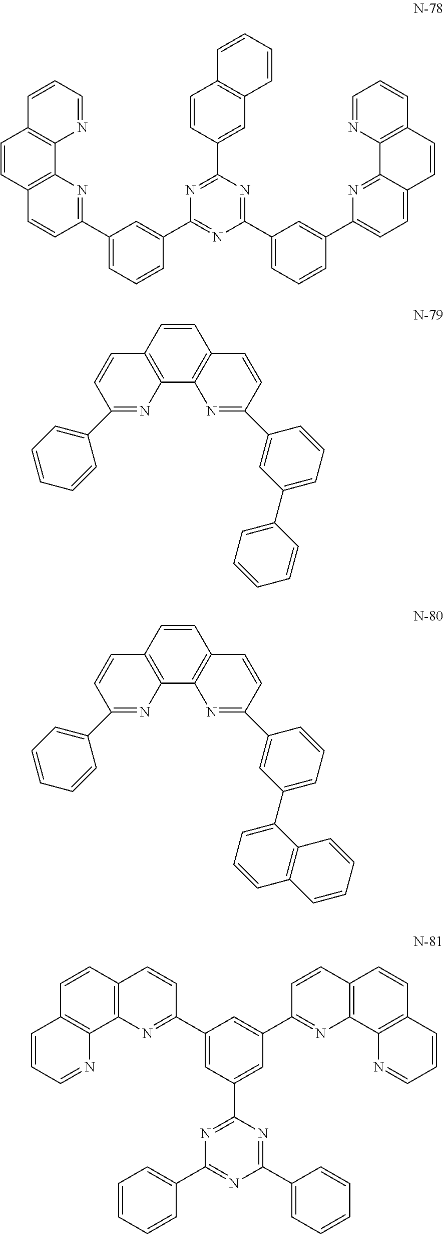 [SMILES: c1cc(-c2ccc3ccc4cccnc4c3n2)cc(-c2nc(-c3cccc(-c4ccc5ccc6cccnc6c5n4)c3)nc(-c3ccc4ccccc4c3)n2)c1 ; c1ccc(-c2cccc(-c3ccc4ccc5ccc(-c6ccccc6)nc5c4n3)c2)cc1 ; c1ccc(-c2ccc3ccc4ccc(-c5cccc(-c6cccc7ccccc67)c5)nc4c3n2)cc1 ; c1ccc(-c2nc(-c3ccccc3)nc(-c3cc(-c4ccc5ccc6cccnc6c5n4)cc(-c4ccc5ccc6cccnc6c5n4)c3)n2)cc1] 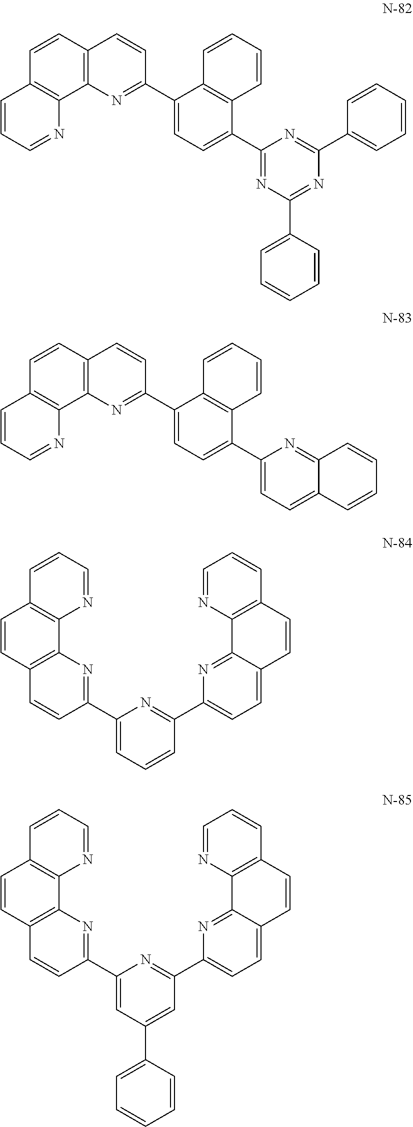 [SMILES: c1ccc(-c2nc(-c3ccccc3)nc(-c3ccc(-c4ccc5ccc6cccnc6c5n4)c4ccccc34)n2)cc1 ; c1ccc2nc(-c3ccc(-c4ccc5ccc6cccnc6c5n4)c4ccccc34)ccc2c1 ; c1cc(-c2ccc3ccc4cccnc4c3n2)nc(-c2ccc3ccc4cccnc4c3n2)c1 ; c1ccc(-c2cc(-c3ccc4ccc5cccnc5c4n3)nc(-c3ccc4ccc5cccnc5c4n3)c2)cc1] 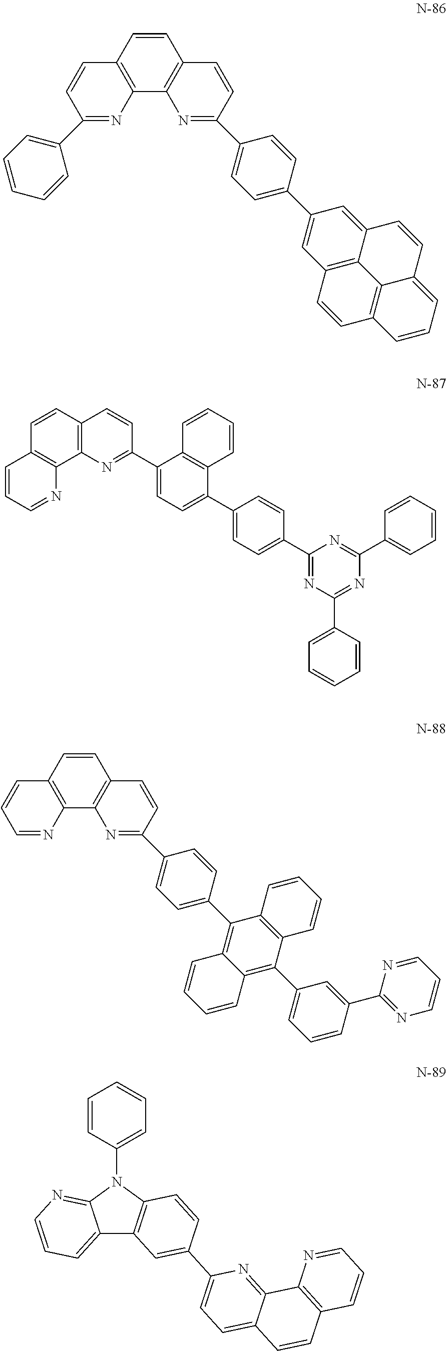 [SMILES: c1ccc(-c2ccc3ccc4ccc(-c5ccc(-c6cc7ccc8cccc9ccc(c6)c7c89)cc5)nc4c3n2)cc1 ; c1ccc(-c2nc(-c3ccccc3)nc(-c3ccc(-c4ccc(-c5ccc6ccc7cccnc7c6n5)c5ccccc45)cc3)n2)cc1 ; c1cnc(-c2cccc(-c3c4ccccc4c(-c4ccc(-c5ccc6ccc7cccnc7c6n5)cc4)c4ccccc34)c2)nc1 ; c1ccc(-n2c3ccc(-c4ccc5ccc6cccnc6c5n4)cc3c3cccnc32)cc1] 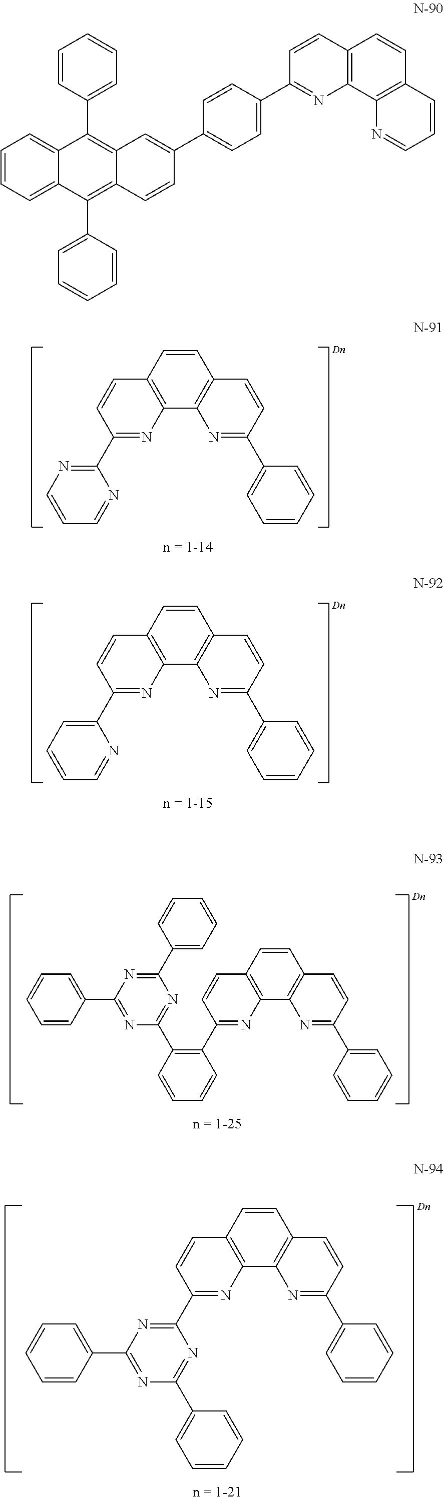 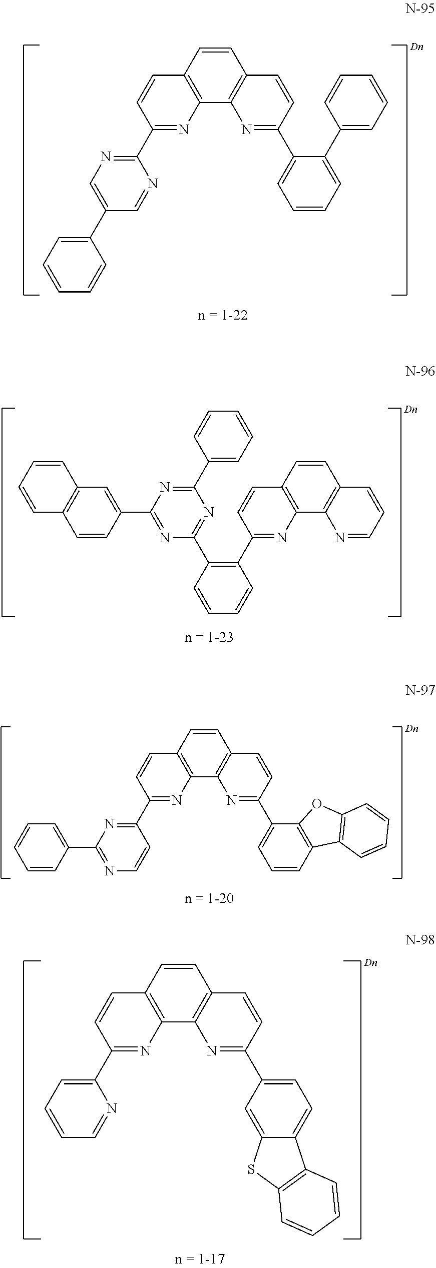 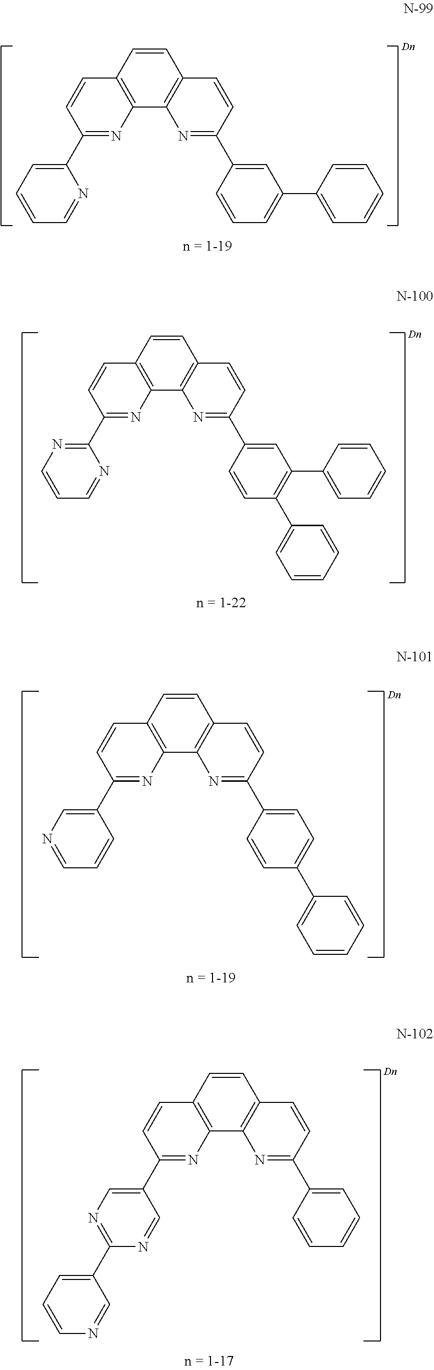 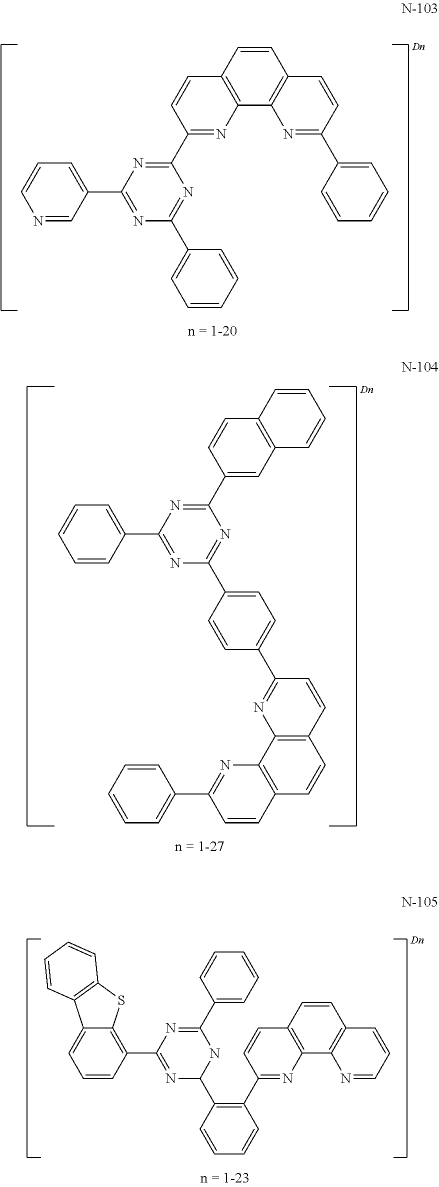 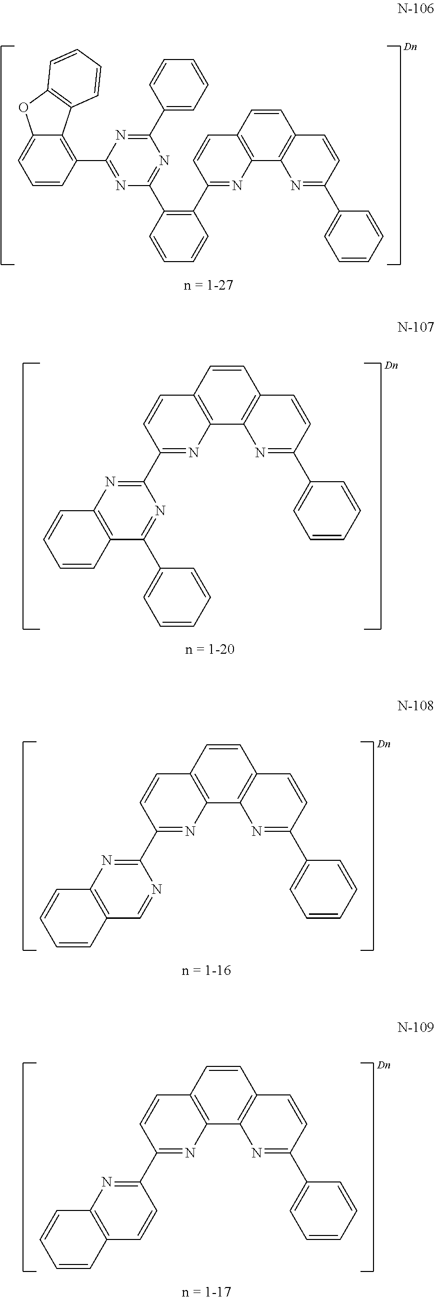 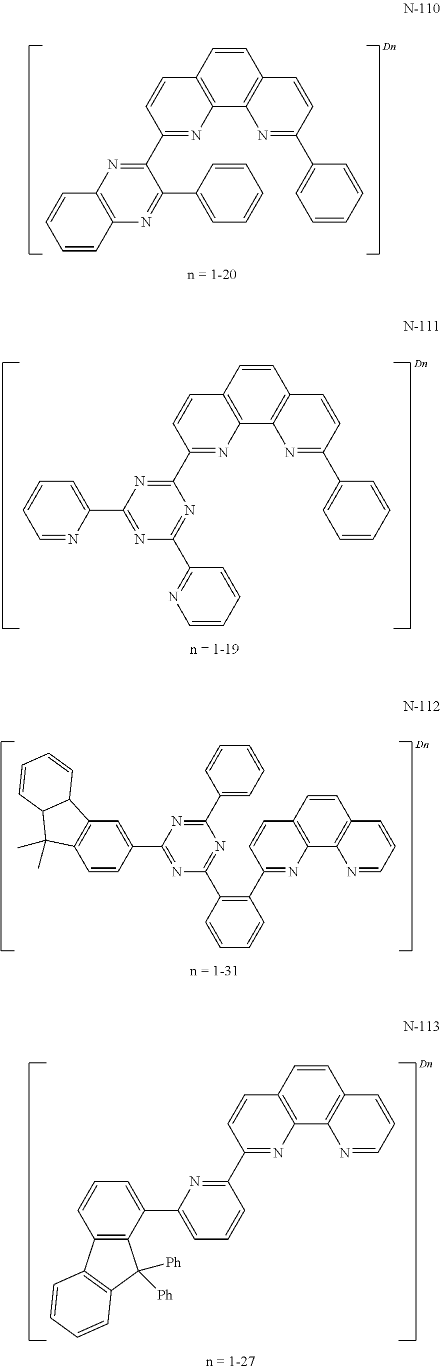 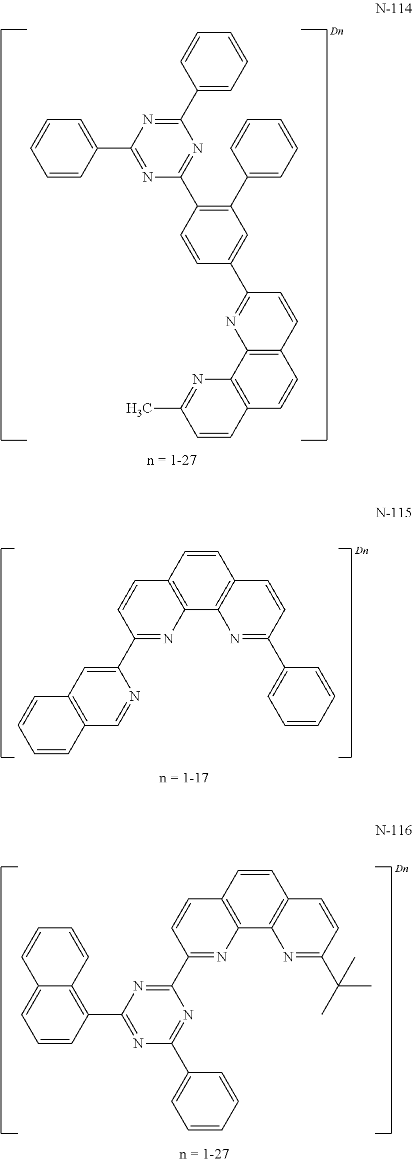 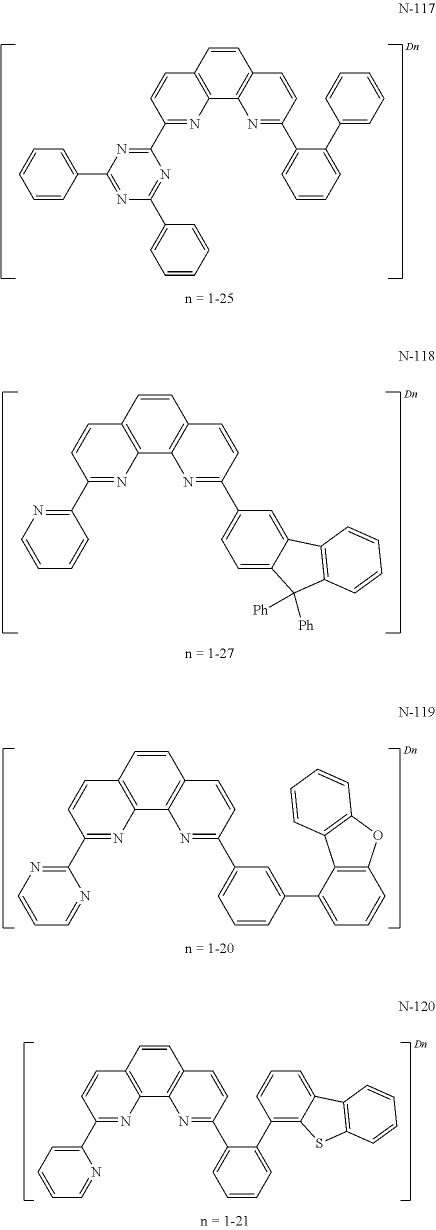 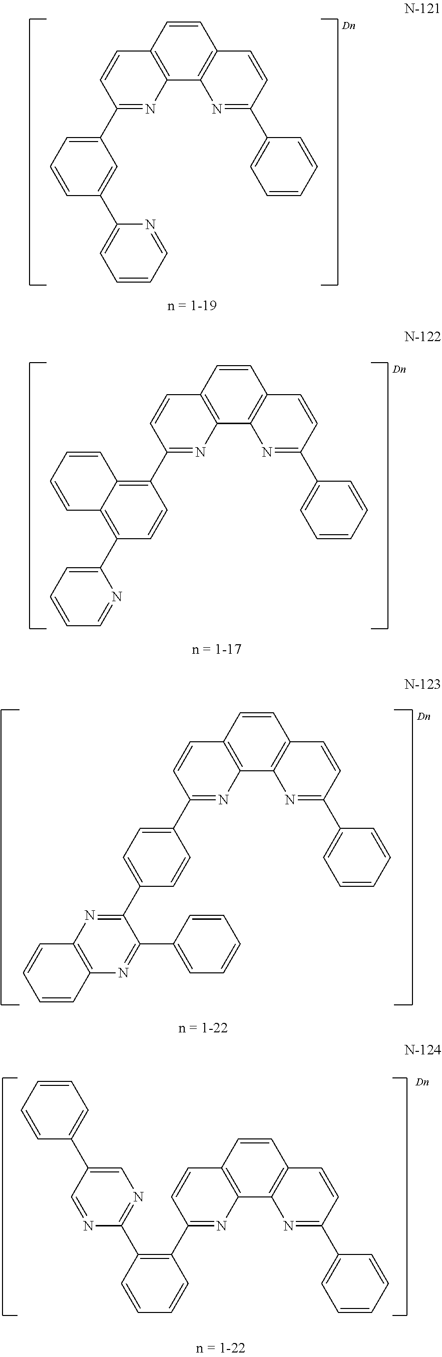 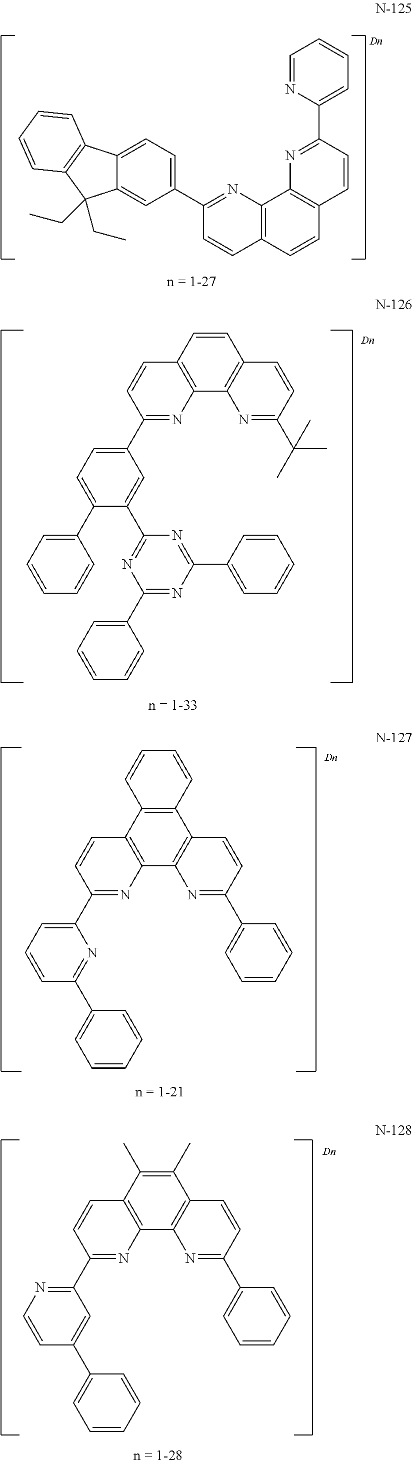 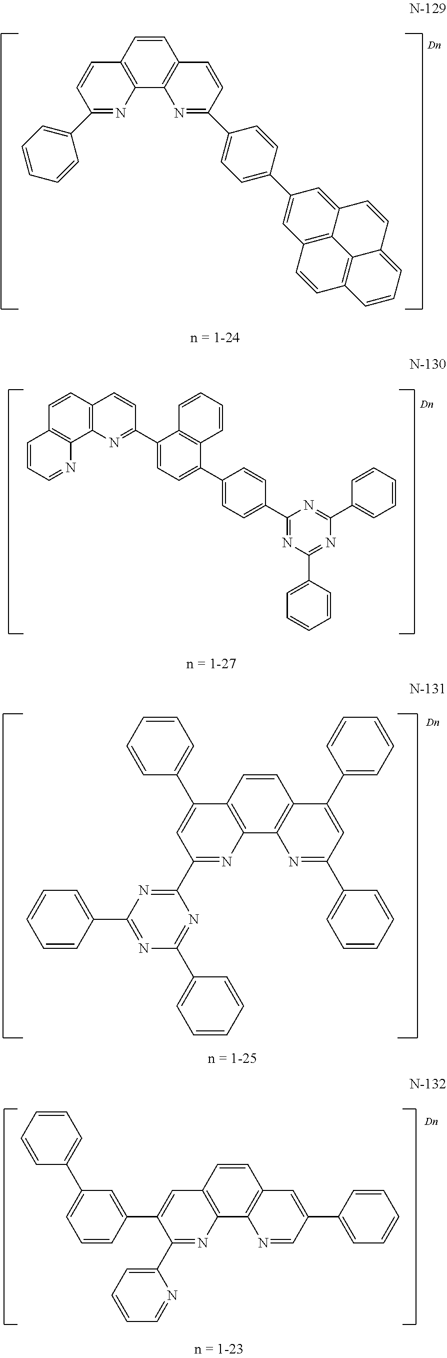 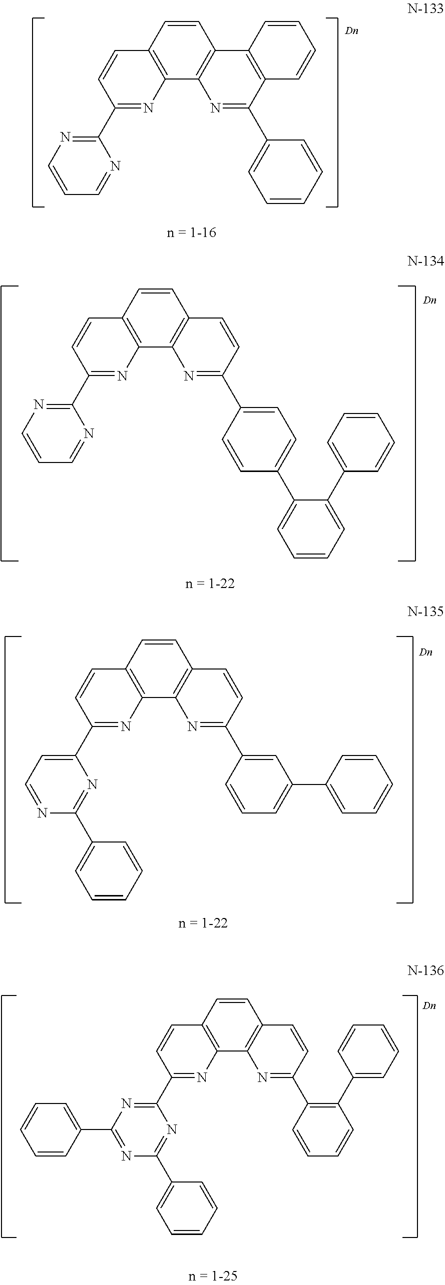 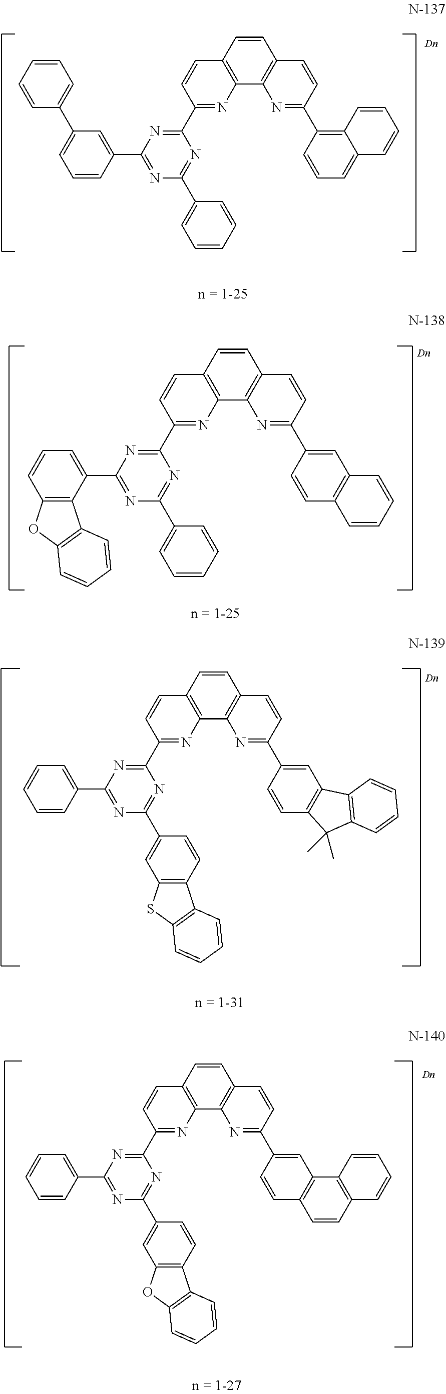 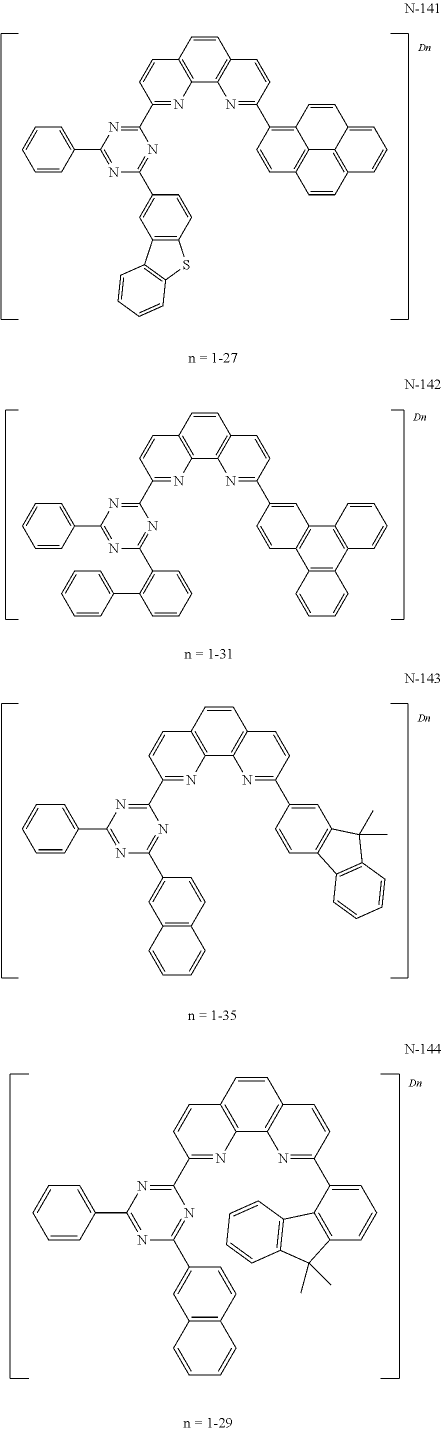 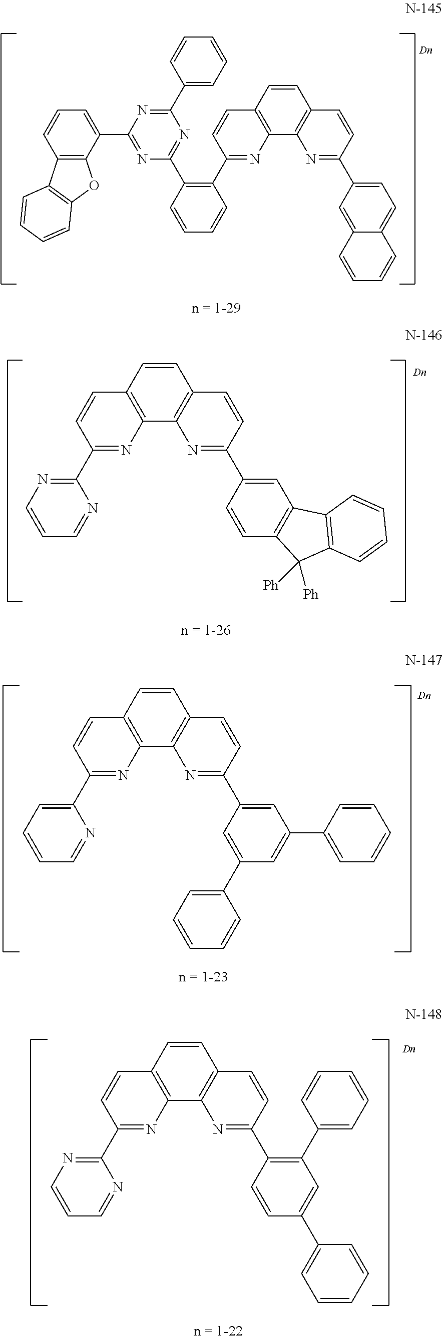 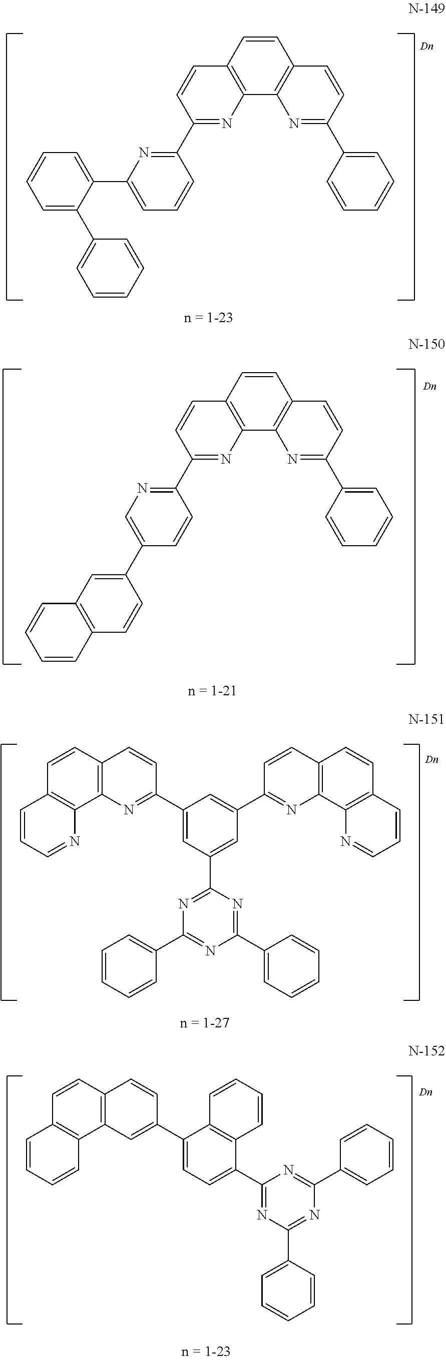 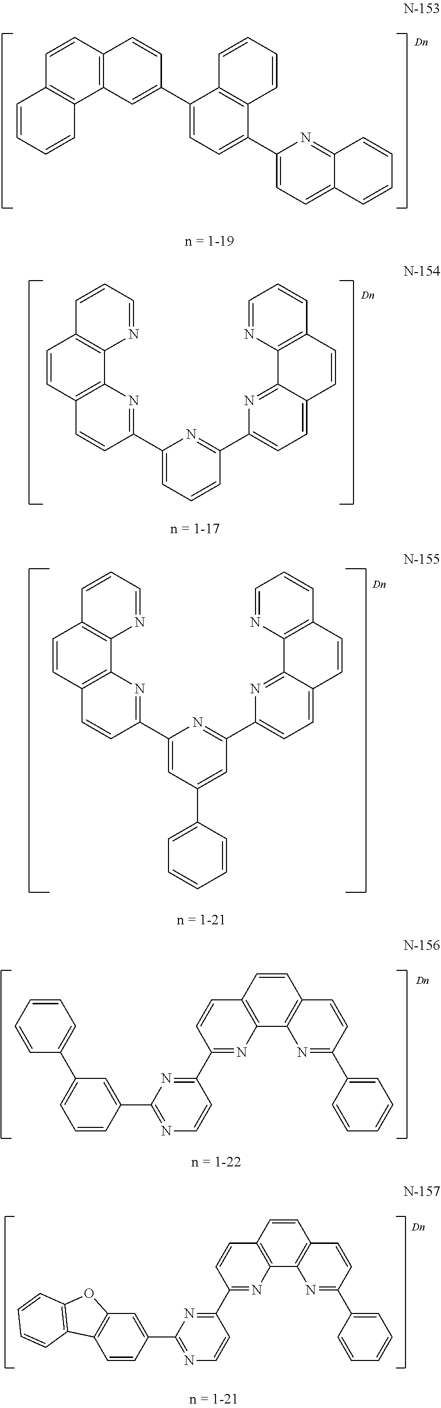 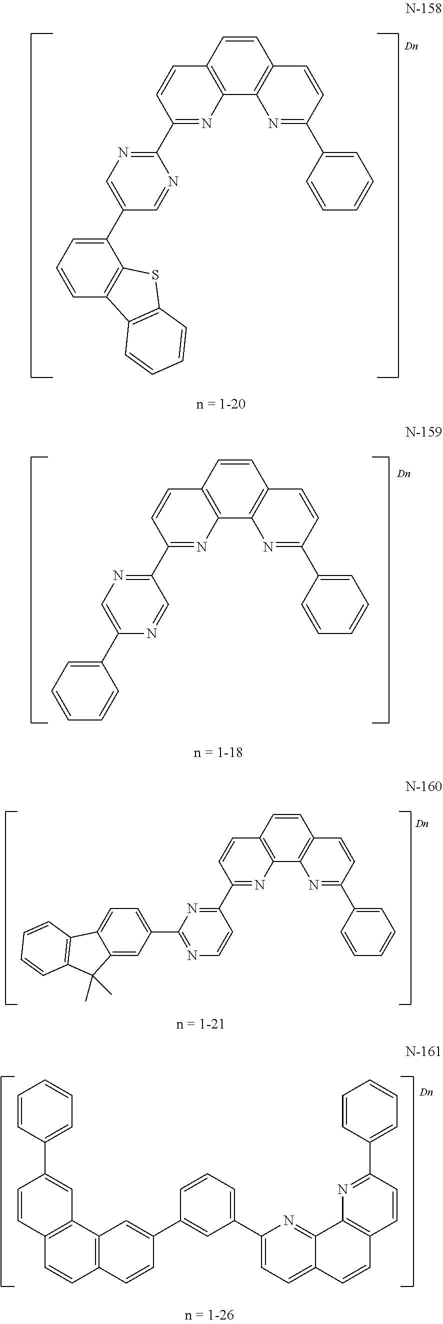 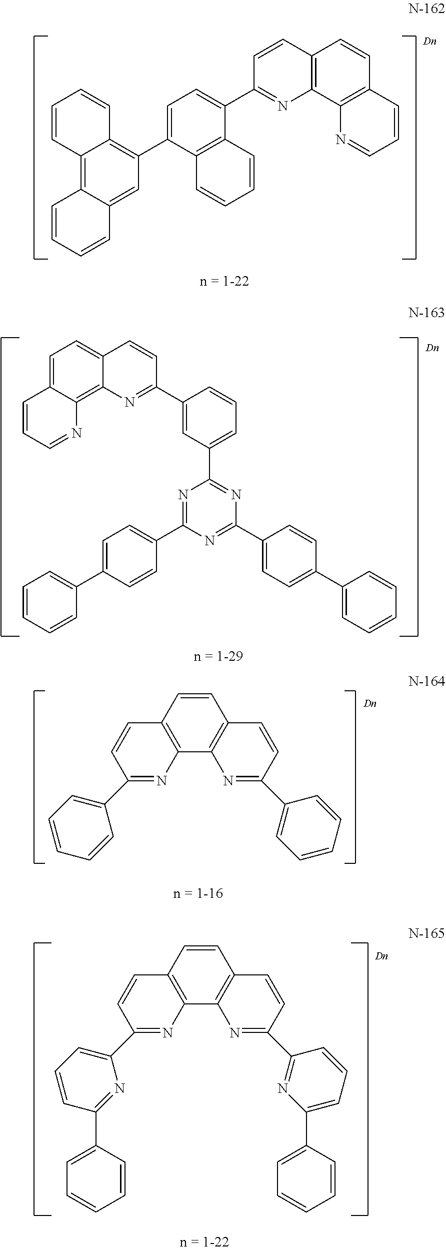 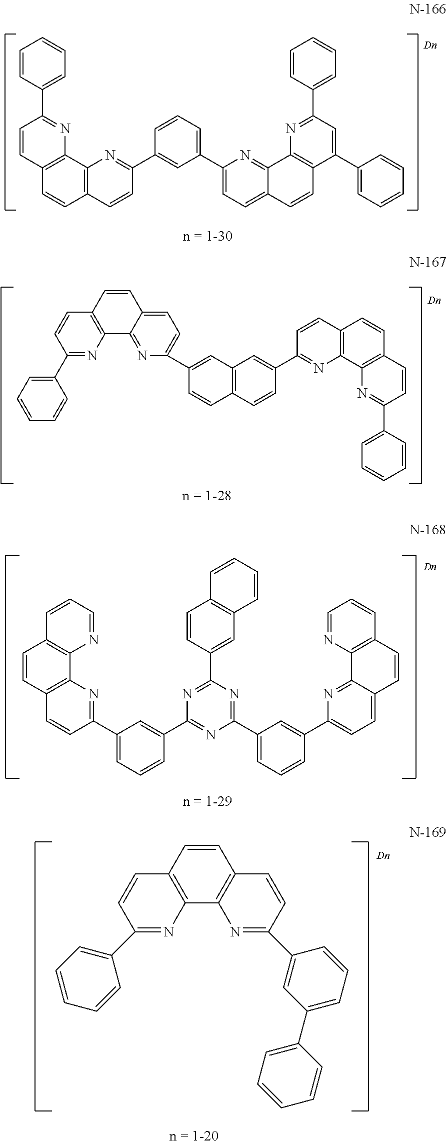 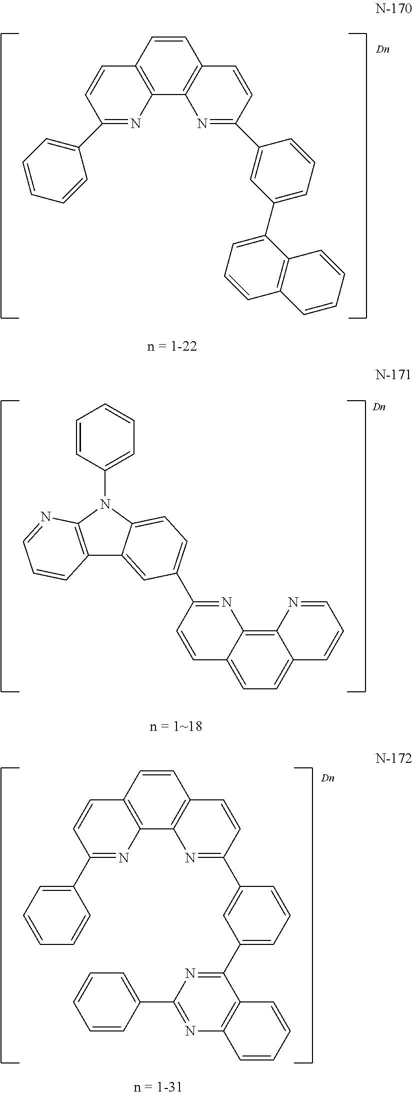 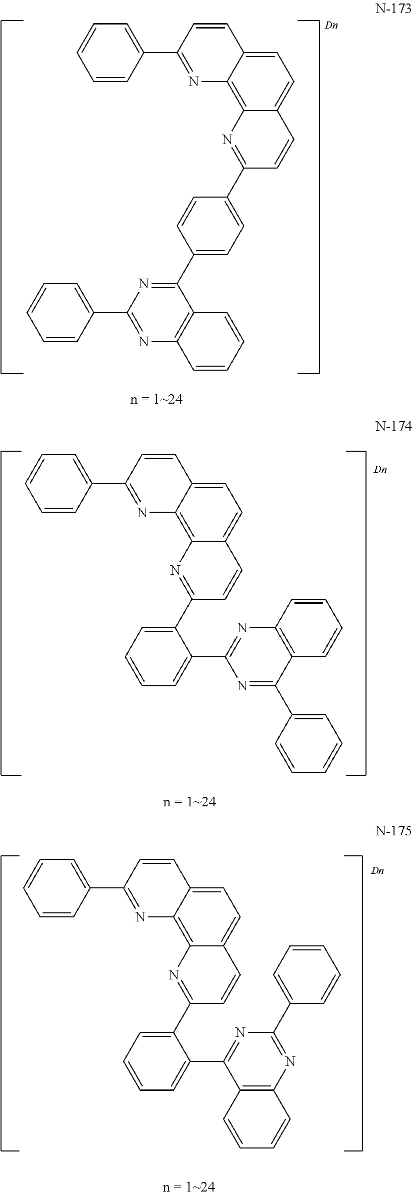 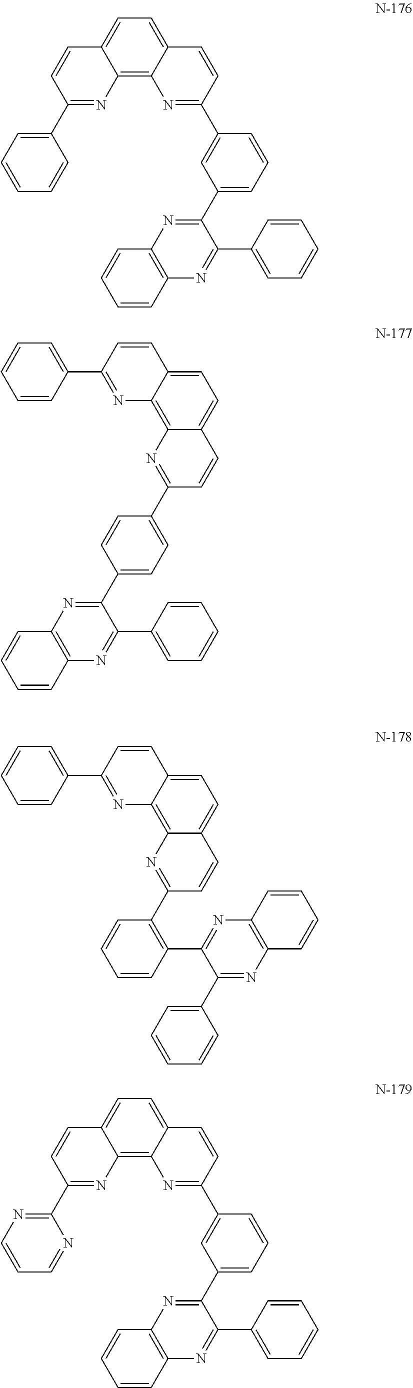 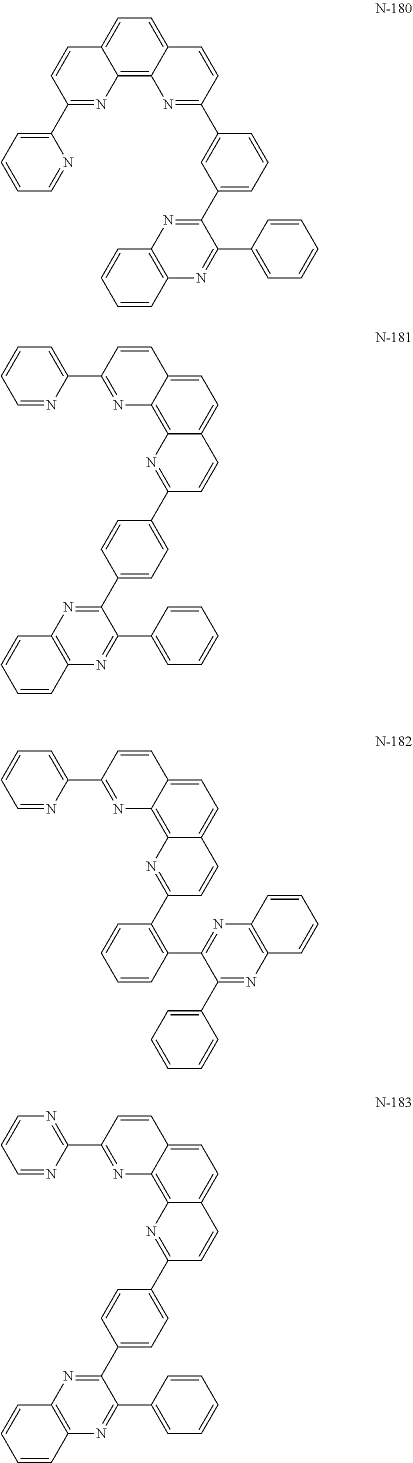 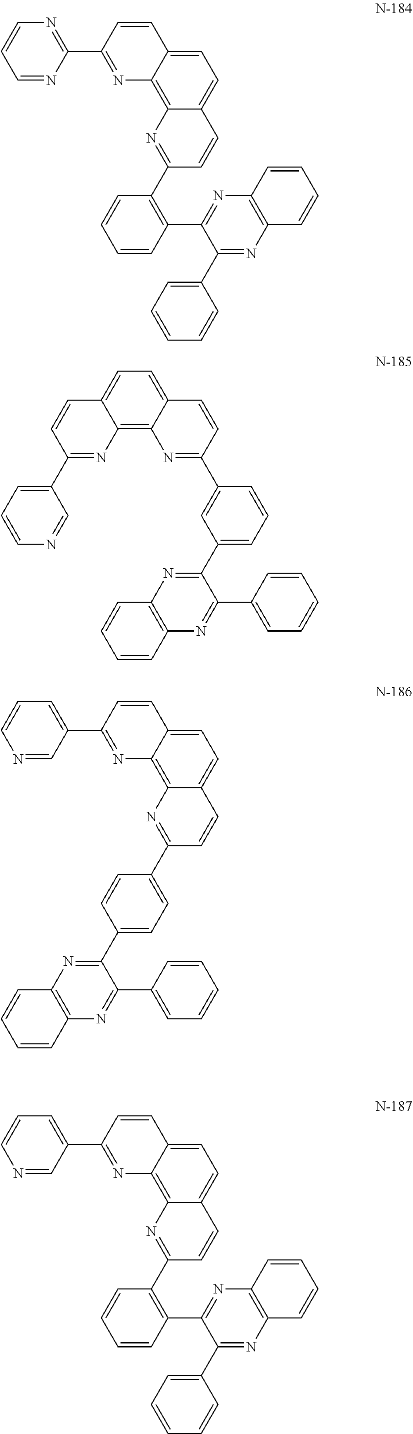 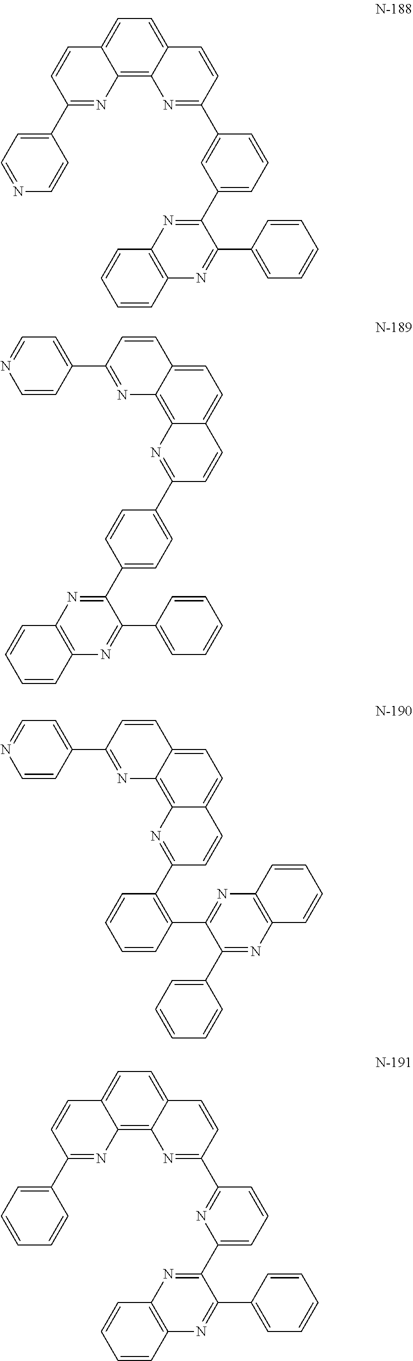 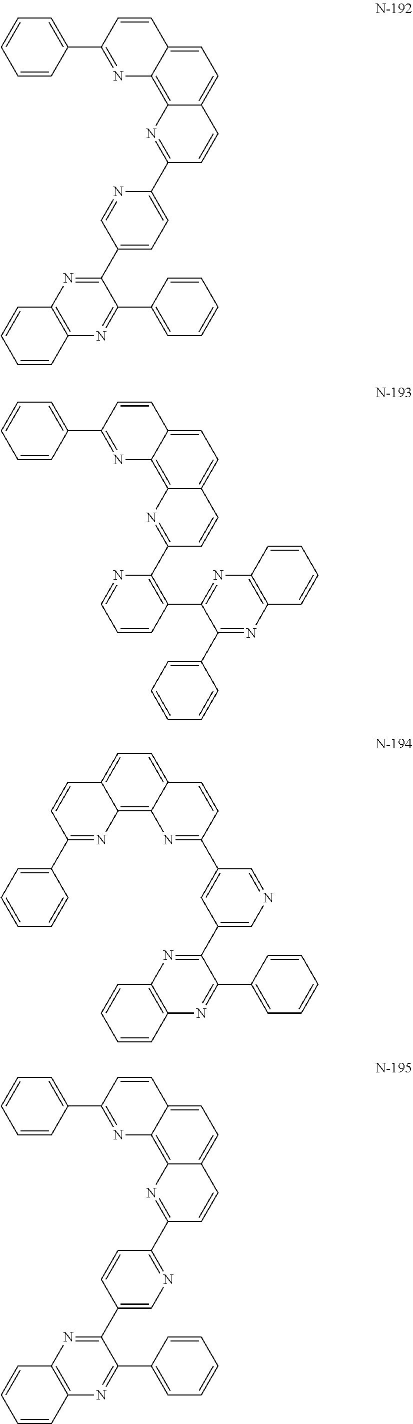 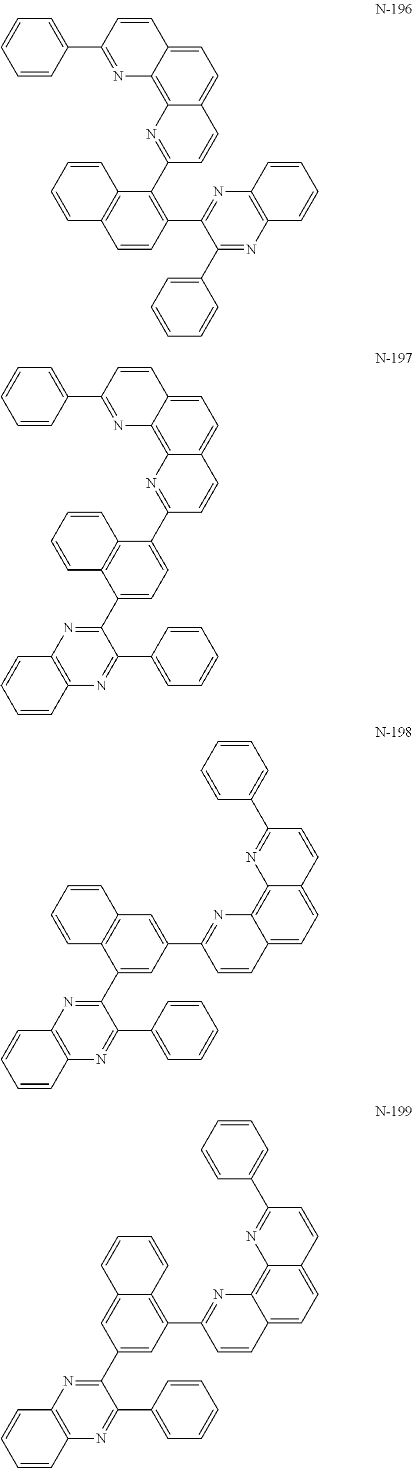 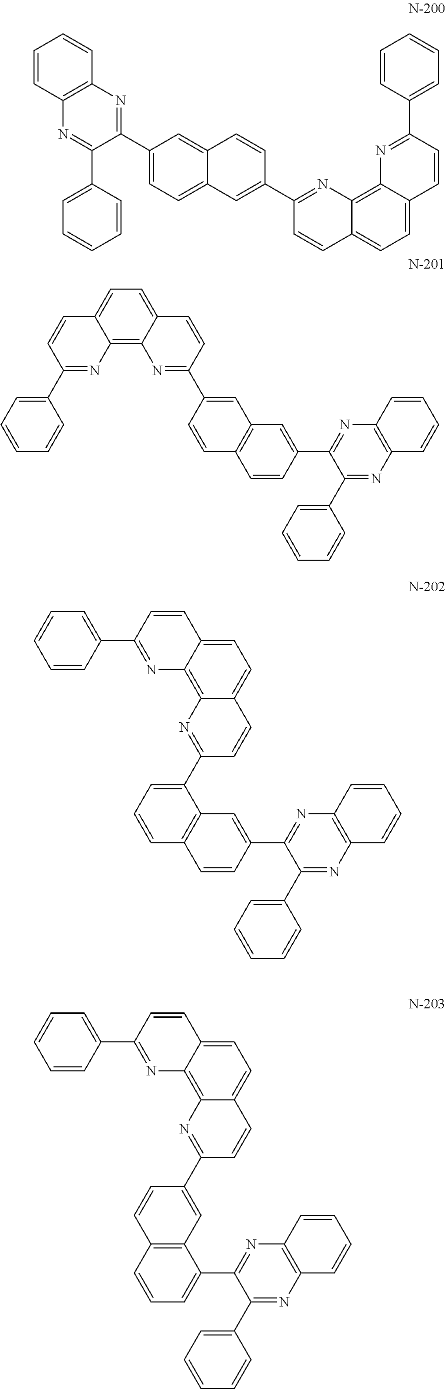 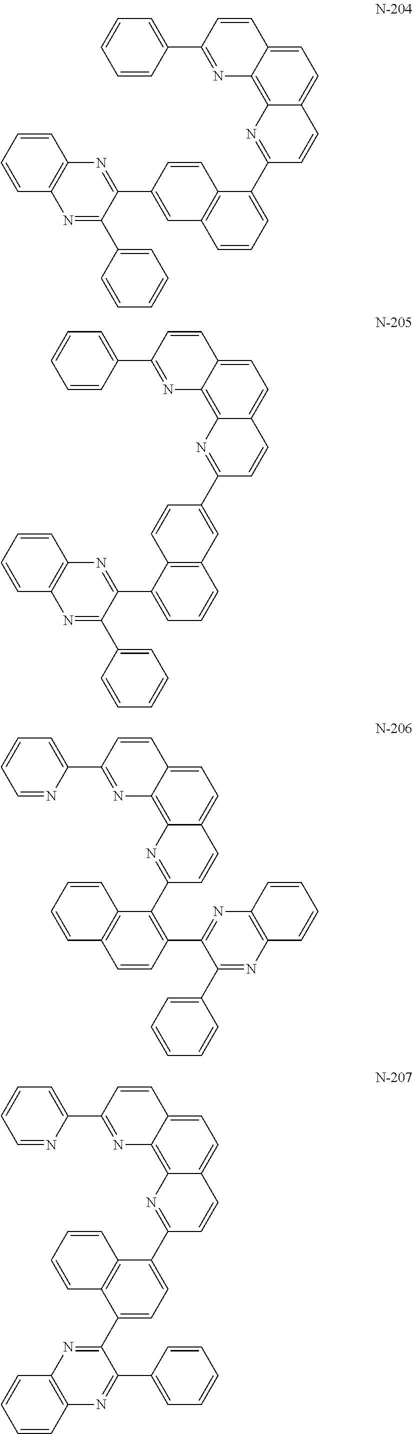 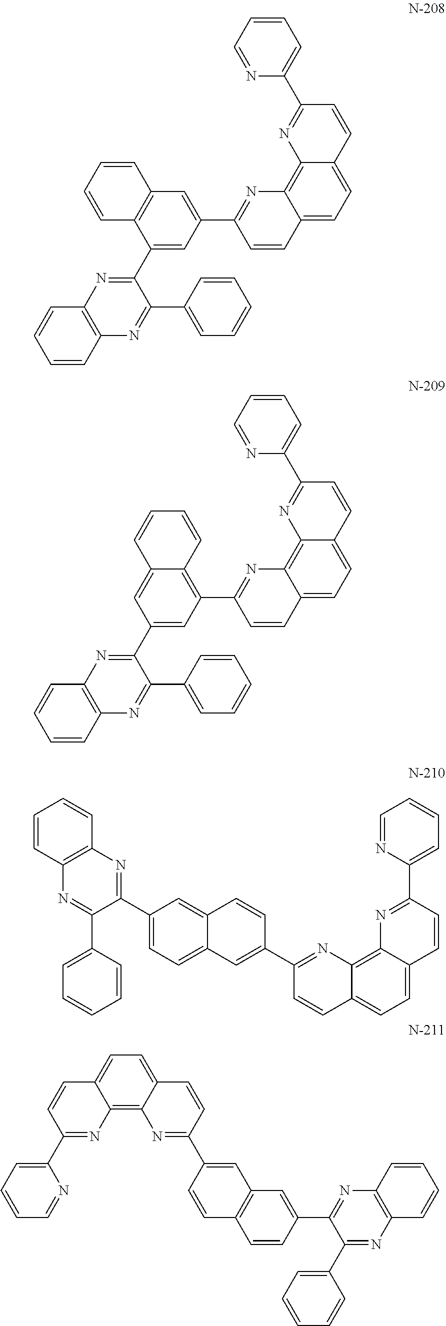 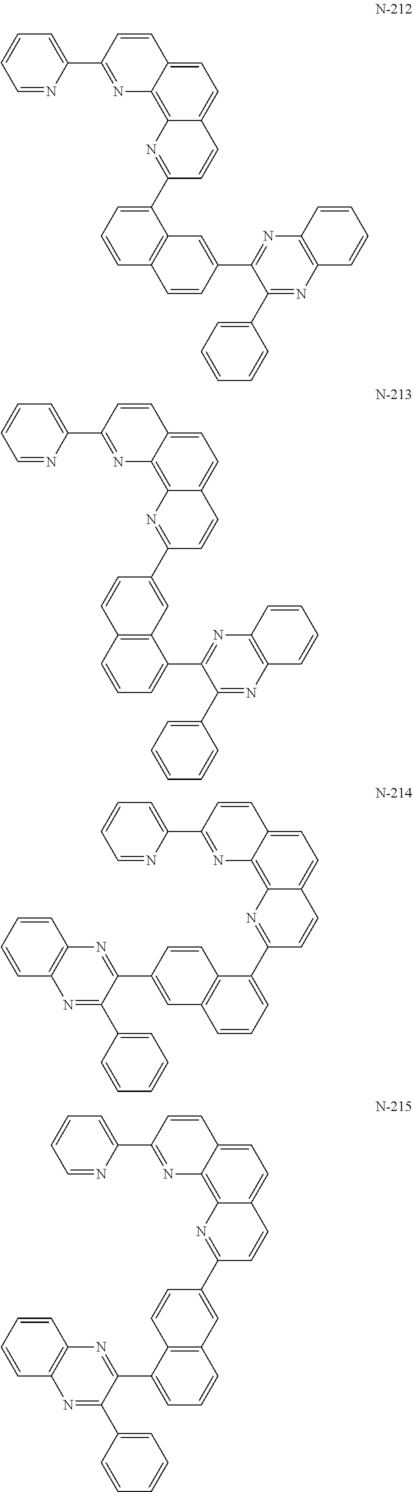 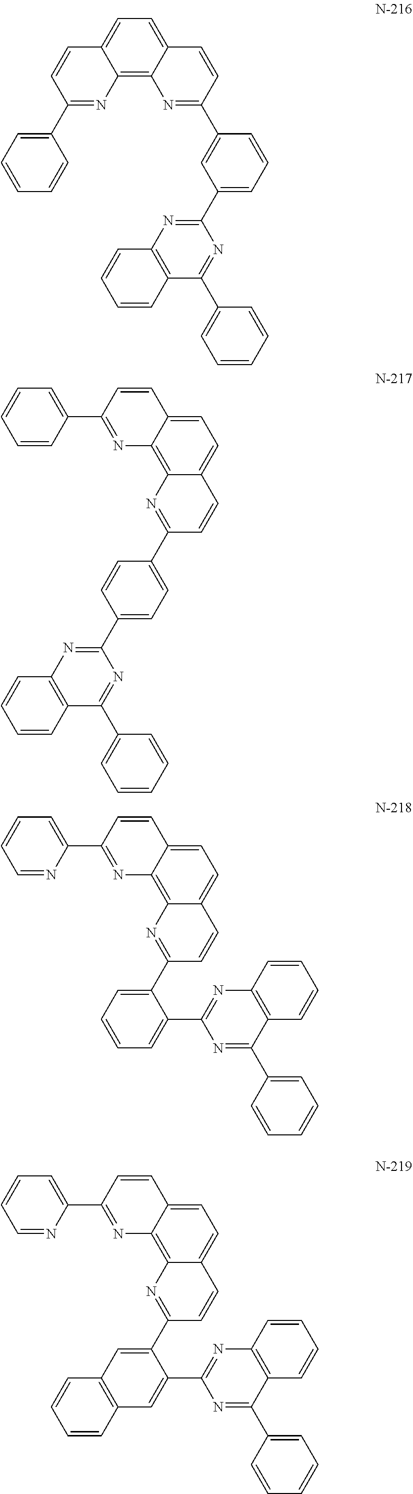 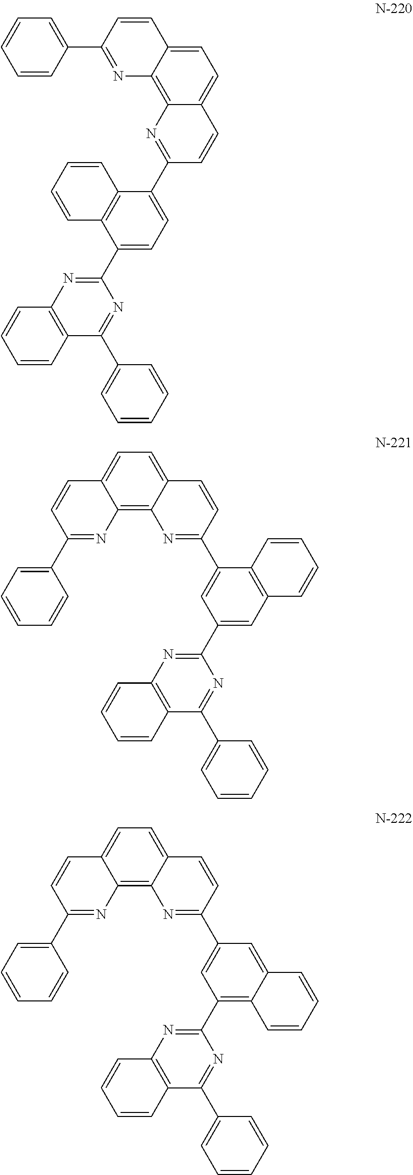 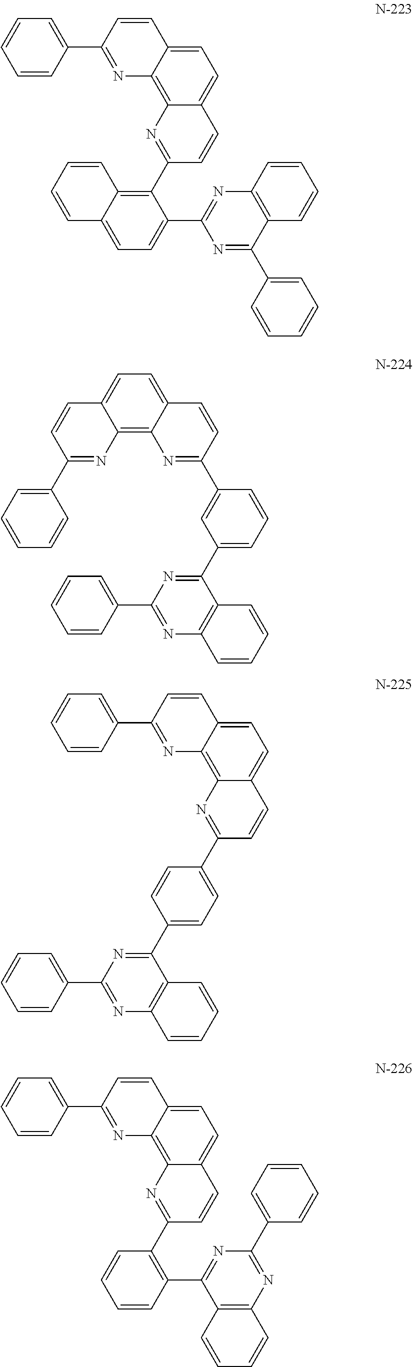 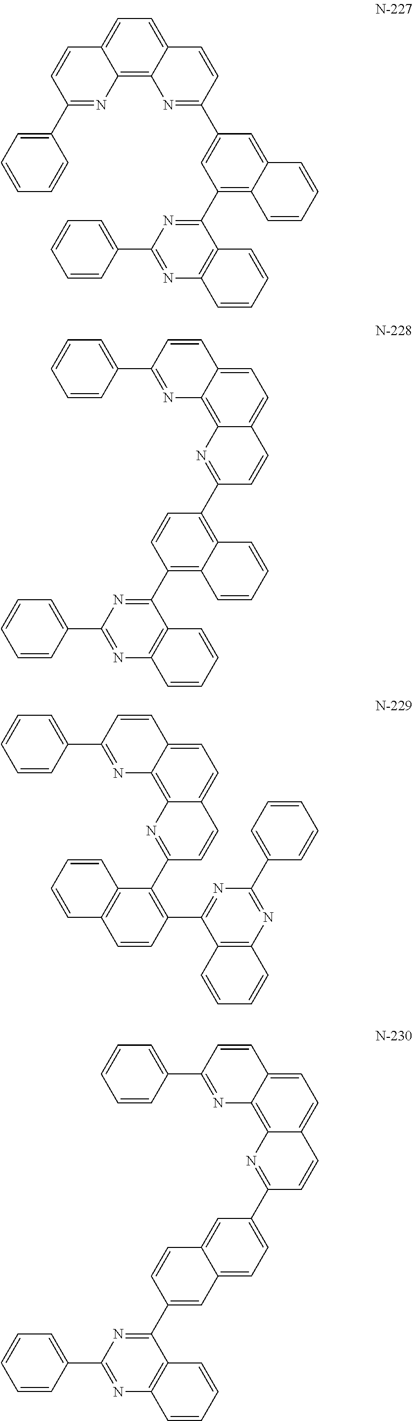 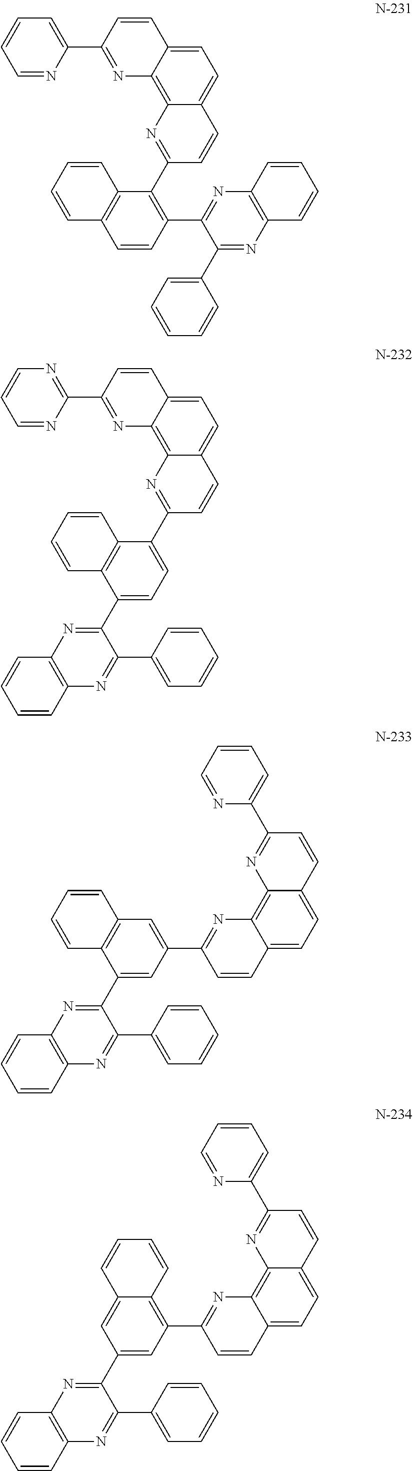 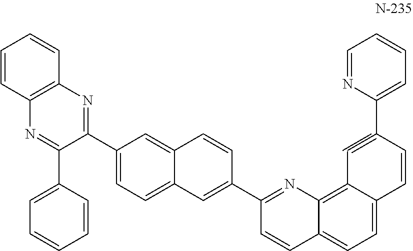 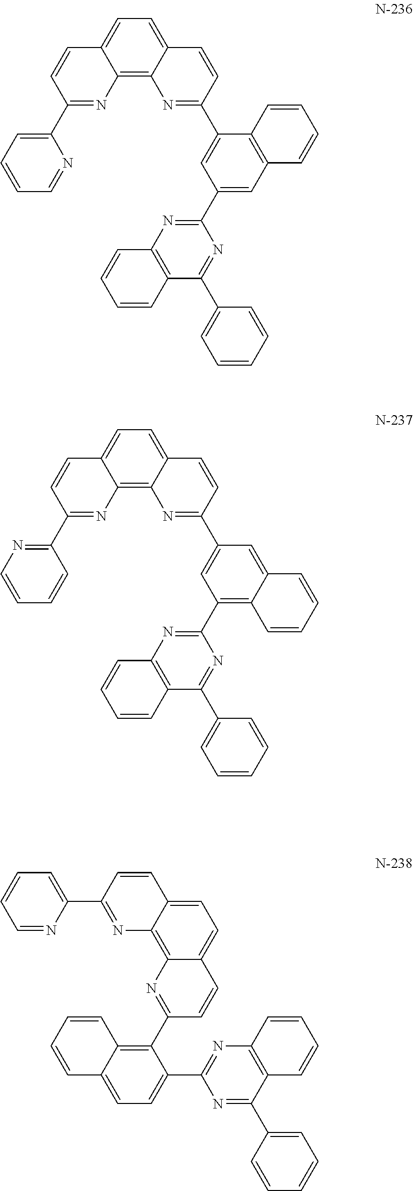 [SMILES: c1ccc(-c2nc(-c3cc(-c4ccc5ccc6ccc(-c7ccccn7)nc6c5n4)c4ccccc4c3)nc3ccccc23)cc1 ; c1ccc(-c2nc(-c3cc(-c4ccc5ccc6ccc(-c7ccccn7)nc6c5n4)cc4ccccc34)nc3ccccc23)cc1 ; c1ccc(-c2nc(-c3ccc4ccccc4c3-c3ccc4ccc5ccc(-c6ccccn6)nc5c4n3)nc3ccccc23)cc1] 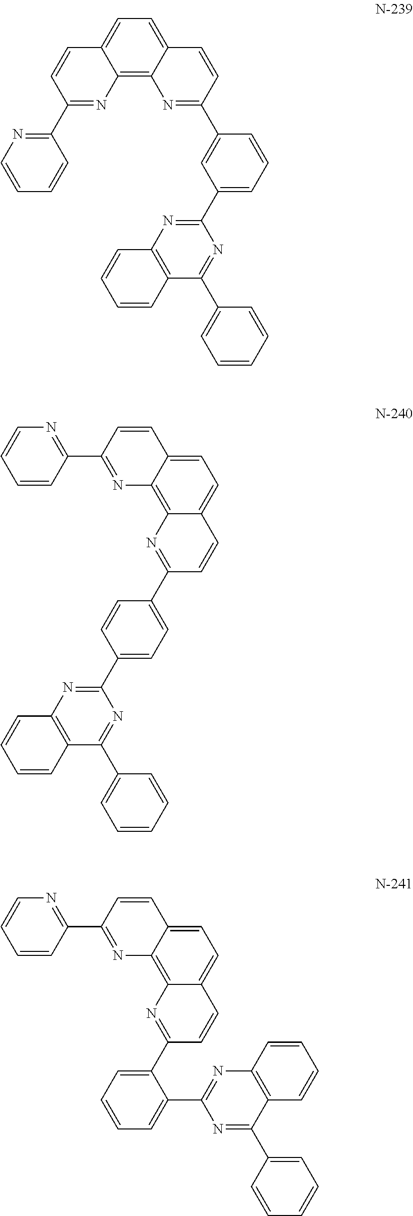 [SMILES: c1ccc(-c2nc(-c3cccc(-c4ccc5ccc6ccc(-c7ccccn7)nc6c5n4)c3)nc3ccccc23)cc1 ; c1ccc(-c2nc(-c3ccc(-c4ccc5ccc6ccc(-c7ccccn7)nc6c5n4)cc3)nc3ccccc23)cc1 ; c1ccc(-c2nc(-c3ccccc3-c3ccc4ccc5ccc(-c6ccccn6)nc5c4n3)nc3ccccc23)cc1] 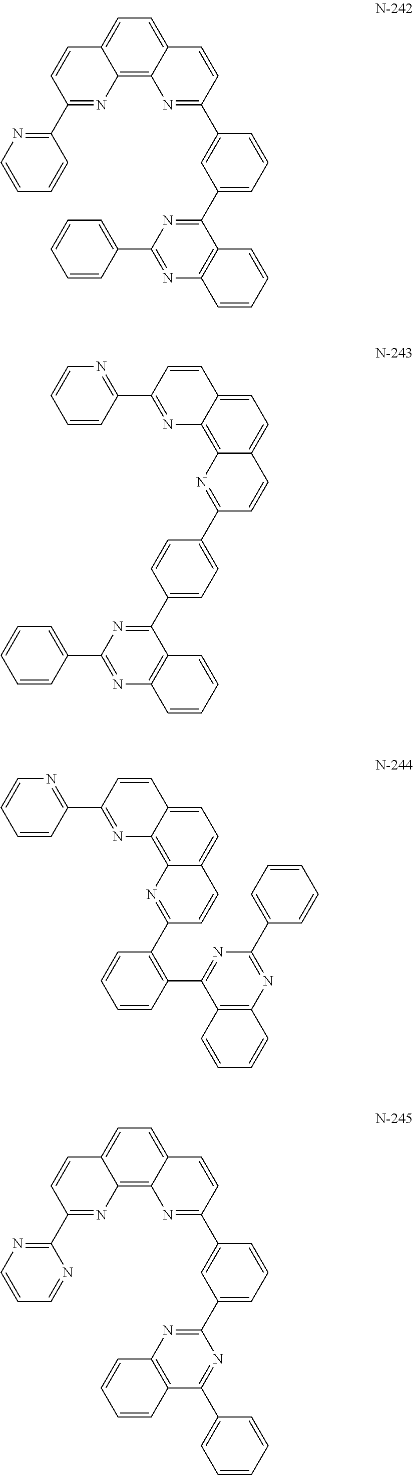 [SMILES: c1ccc(-c2nc(-c3cccc(-c4ccc5ccc6ccc(-c7ccccn7)nc6c5n4)c3)c3ccccc3n2)cc1 ; c1ccc(-c2nc(-c3ccc(-c4ccc5ccc6ccc(-c7ccccn7)nc6c5n4)cc3)c3ccccc3n2)cc1 ; c1ccc(-c2nc(-c3ccccc3-c3ccc4ccc5ccc(-c6ccccn6)nc5c4n3)c3ccccc3n2)cc1 ; c1ccc(-c2nc(-c3cccc(-c4ccc5ccc6ccc(-c7ncccn7)nc6c5n4)c3)nc3ccccc23)cc1] 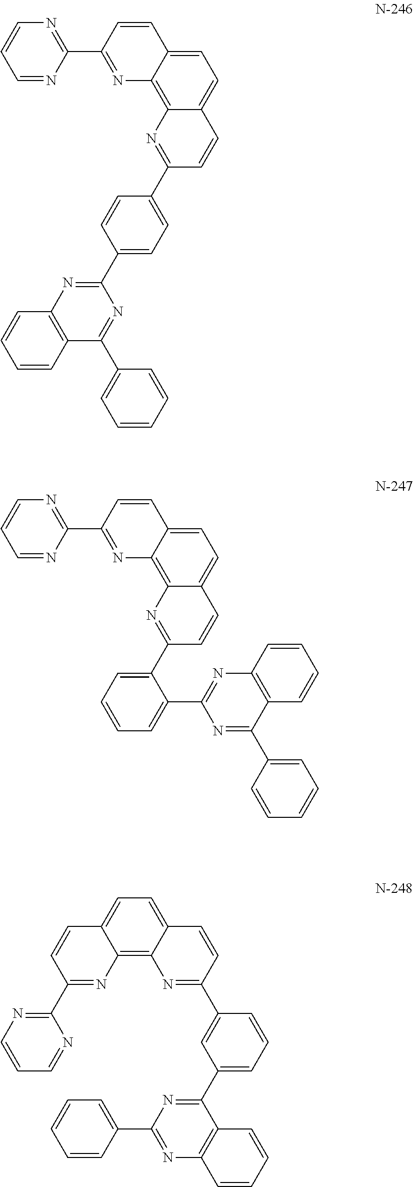 [SMILES: c1ccc(-c2nc(-c3ccc(-c4ccc5ccc6ccc(-c7ncccn7)nc6c5n4)cc3)nc3ccccc23)cc1 ; c1ccc(-c2nc(-c3ccccc3-c3ccc4ccc5ccc(-c6ncccn6)nc5c4n3)nc3ccccc23)cc1 ; c1ccc(-c2nc(-c3cccc(-c4ccc5ccc6ccc(-c7ncccn7)nc6c5n4)c3)c3ccccc3n2)cc1] 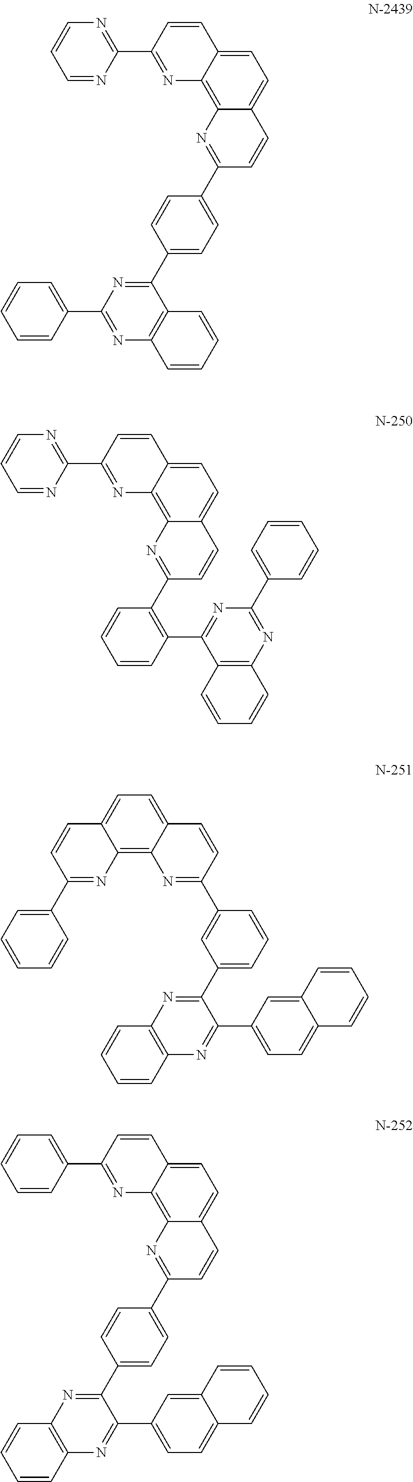 [SMILES: c1ccc(-c2nc(-c3ccc(-c4ccc5ccc6ccc(-c7ncccn7)nc6c5n4)cc3)c3ccccc3n2)cc1 ; c1ccc(-c2nc(-c3ccccc3-c3ccc4ccc5ccc(-c6ncccn6)nc5c4n3)c3ccccc3n2)cc1 ; c1ccc(-c2ccc3ccc4ccc(-c5cccc(-c6nc7ccccc7nc6-c6ccc7ccccc7c6)c5)nc4c3n2)cc1 ; c1ccc(-c2ccc3ccc4ccc(-c5ccc(-c6nc7ccccc7nc6-c6ccc7ccccc7c6)cc5)nc4c3n2)cc1] 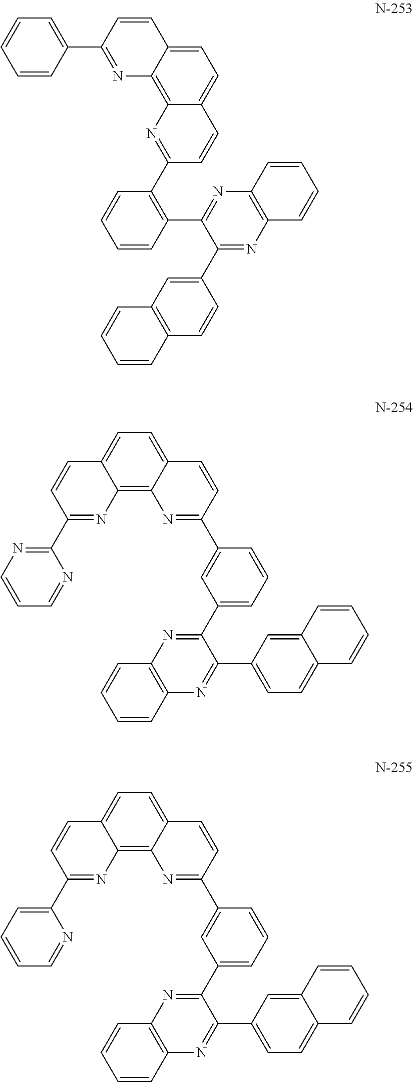 [SMILES: c1ccc(-c2ccc3ccc4ccc(-c5ccccc5-c5nc6ccccc6nc5-c5ccc6ccccc6c5)nc4c3n2)cc1 ; c1cnc(-c2ccc3ccc4ccc(-c5cccc(-c6nc7ccccc7nc6-c6ccc7ccccc7c6)c5)nc4c3n2)nc1 ; c1ccc(-c2ccc3ccc4ccc(-c5cccc(-c6nc7ccccc7nc6-c6ccc7ccccc7c6)c5)nc4c3n2)nc1] 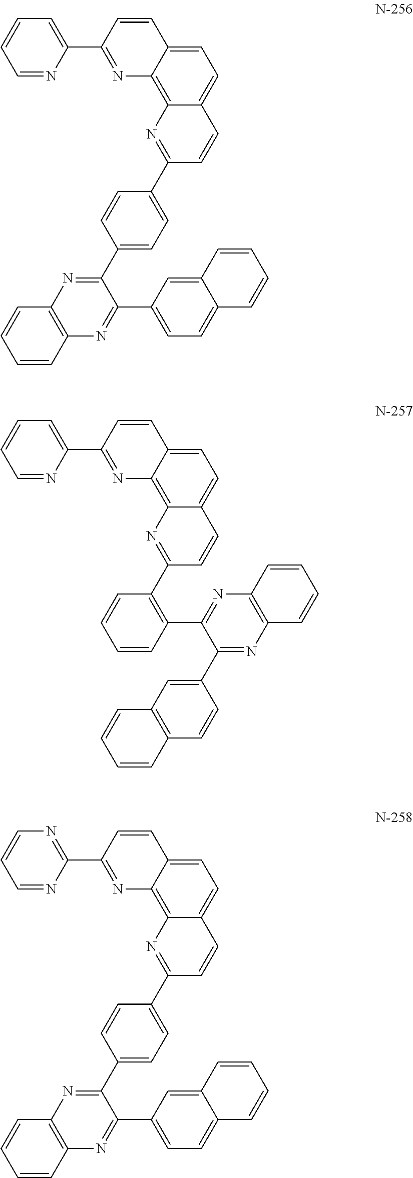 [SMILES: c1ccc(-c2ccc3ccc4ccc(-c5ccc(-c6nc7ccccc7nc6-c6ccc7ccccc7c6)cc5)nc4c3n2)nc1 ; c1ccc(-c2ccc3ccc4ccc(-c5ccccc5-c5nc6ccccc6nc5-c5ccc6ccccc6c5)nc4c3n2)nc1 ; c1cnc(-c2ccc3ccc4ccc(-c5ccc(-c6nc7ccccc7nc6-c6ccc7ccccc7c6)cc5)nc4c3n2)nc1] 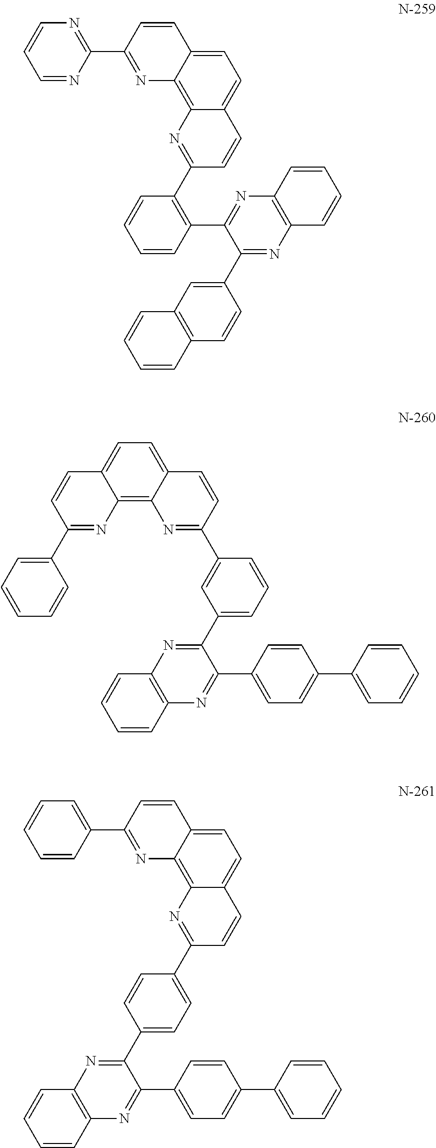 [SMILES: c1cnc(-c2ccc3ccc4ccc(-c5ccccc5-c5nc6ccccc6nc5-c5ccc6ccccc6c5)nc4c3n2)nc1 ; c1ccc(-c2ccc(-c3nc4ccccc4nc3-c3cccc(-c4ccc5ccc6ccc(-c7ccccc7)nc6c5n4)c3)cc2)cc1 ; c1ccc(-c2ccc(-c3nc4ccccc4nc3-c3ccc(-c4ccc5ccc6ccc(-c7ccccc7)nc6c5n4)cc3)cc2)cc1] 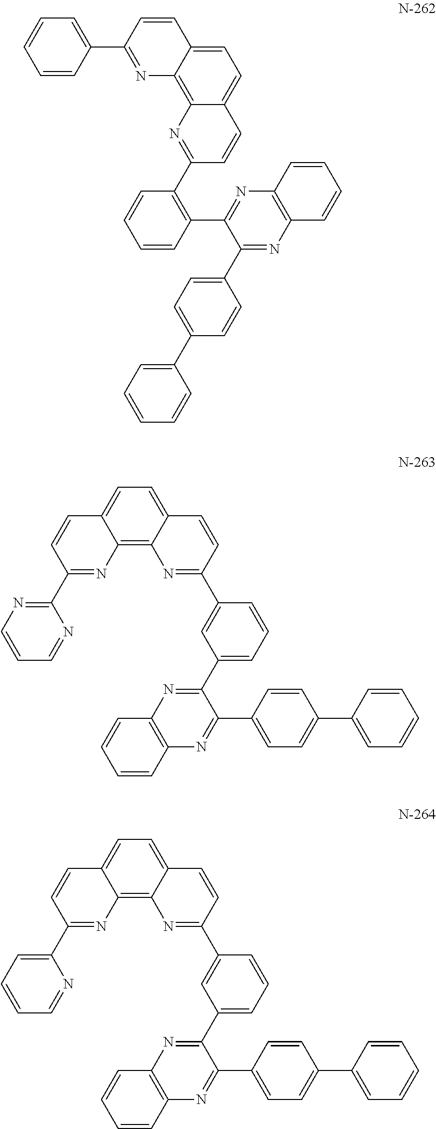 [SMILES: c1ccc(-c2ccc(-c3nc4ccccc4nc3-c3ccccc3-c3ccc4ccc5ccc(-c6ccccc6)nc5c4n3)cc2)cc1 ; c1ccc(-c2ccc(-c3nc4ccccc4nc3-c3cccc(-c4ccc5ccc6ccc(-c7ncccn7)nc6c5n4)c3)cc2)cc1 ; c1ccc(-c2ccc(-c3nc4ccccc4nc3-c3cccc(-c4ccc5ccc6ccc(-c7ccccn7)nc6c5n4)c3)cc2)cc1] 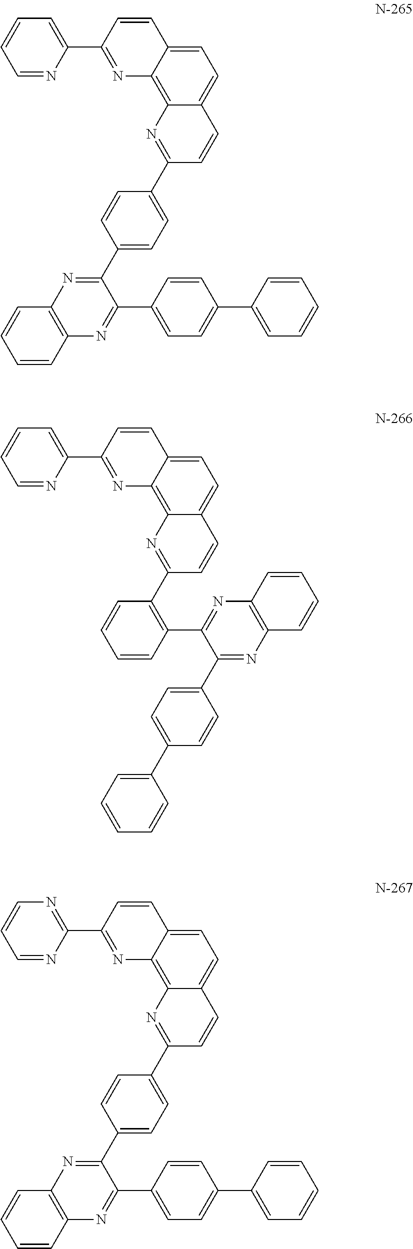 [SMILES: c1ccc(-c2ccc(-c3nc4ccccc4nc3-c3ccc(-c4ccc5ccc6ccc(-c7ccccn7)nc6c5n4)cc3)cc2)cc1 ; c1ccc(-c2ccc(-c3nc4ccccc4nc3-c3ccccc3-c3ccc4ccc5ccc(-c6ccccn6)nc5c4n3)cc2)cc1 ; c1ccc(-c2ccc(-c3nc4ccccc4nc3-c3ccc(-c4ccc5ccc6ccc(-c7ncccn7)nc6c5n4)cc3)cc2)cc1] 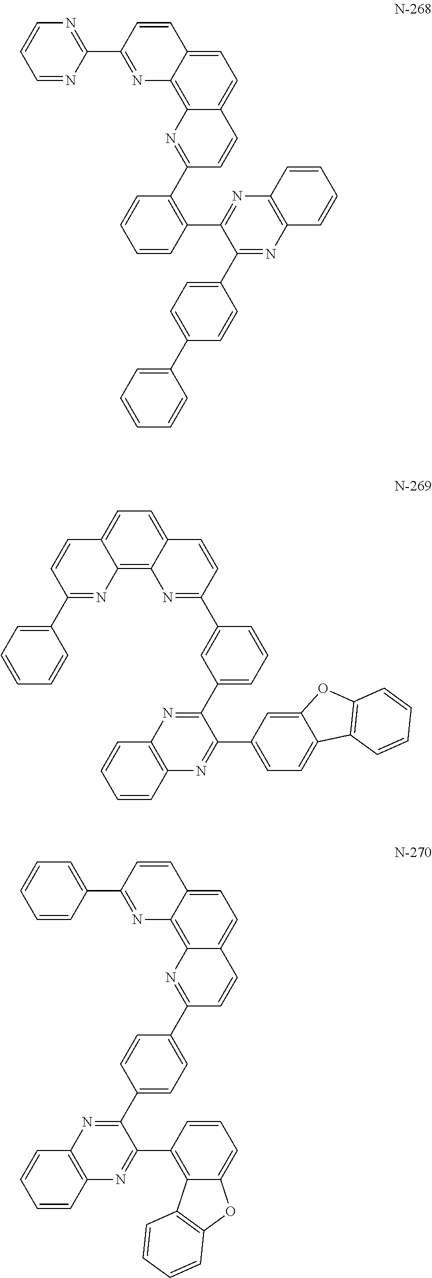 [SMILES: c1ccc(-c2ccc(-c3nc4ccccc4nc3-c3ccccc3-c3ccc4ccc5ccc(-c6ncccn6)nc5c4n3)cc2)cc1 ; c1ccc(-c2ccc3ccc4ccc(-c5cccc(-c6nc7ccccc7nc6-c6ccc7c(c6)oc6ccccc67)c5)nc4c3n2)cc1 ; c1ccc(-c2ccc3ccc4ccc(-c5ccc(-c6nc7ccccc7nc6-c6cccc7oc8ccccc8c67)cc5)nc4c3n2)cc1] 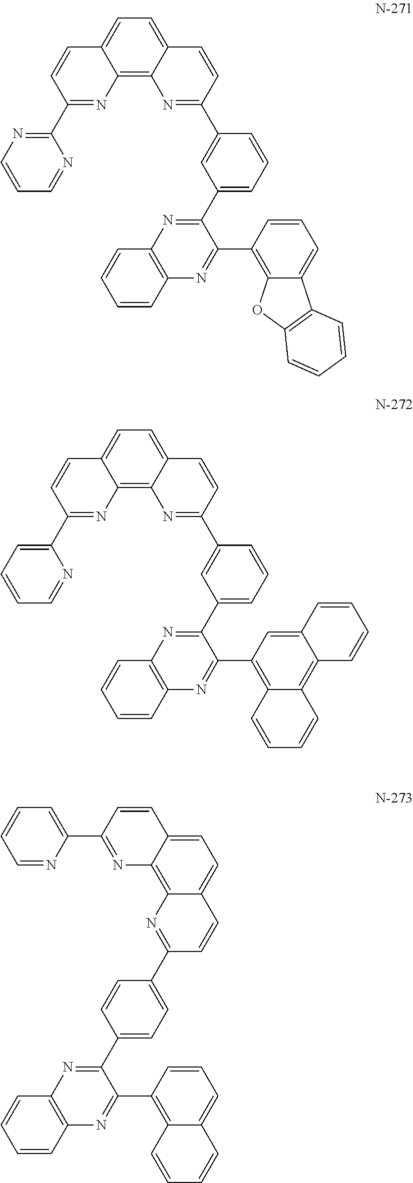 [SMILES: c1cnc(-c2ccc3ccc4ccc(-c5cccc(-c6nc7ccccc7nc6-c6cccc7c6oc6ccccc67)c5)nc4c3n2)nc1 ; c1ccc(-c2ccc3ccc4ccc(-c5cccc(-c6nc7ccccc7nc6-c6cc7ccccc7c7ccccc67)c5)nc4c3n2)nc1 ; c1ccc(-c2ccc3ccc4ccc(-c5ccc(-c6nc7ccccc7nc6-c6cccc7ccccc67)cc5)nc4c3n2)nc1] 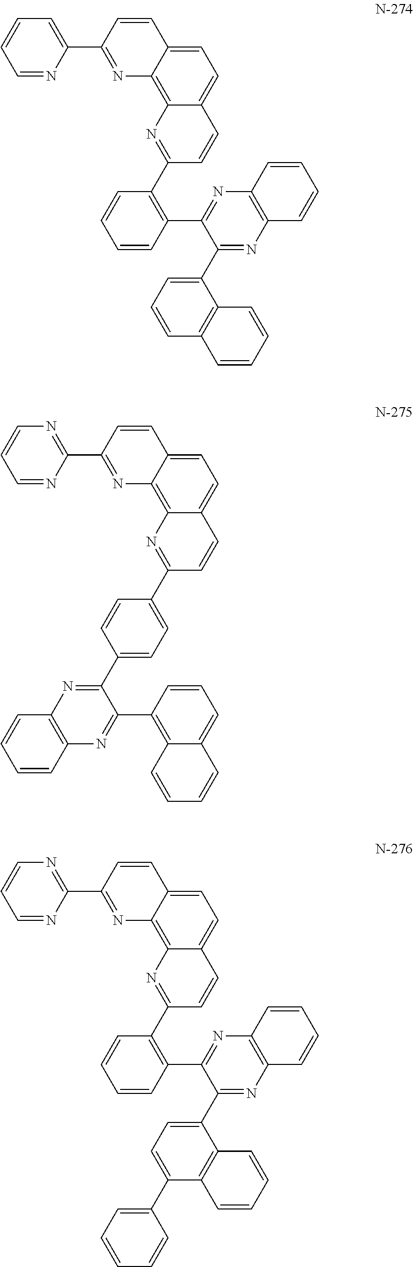 [SMILES: c1ccc(-c2ccc3ccc4ccc(-c5ccccc5-c5nc6ccccc6nc5-c5cccc6ccccc56)nc4c3n2)nc1 ; c1cnc(-c2ccc3ccc4ccc(-c5ccc(-c6nc7ccccc7nc6-c6cccc7ccccc67)cc5)nc4c3n2)nc1 ; c1ccc(-c2ccc(-c3nc4ccccc4nc3-c3ccccc3-c3ccc4ccc5ccc(-c6ncccn6)nc5c4n3)c3ccccc23)cc1] 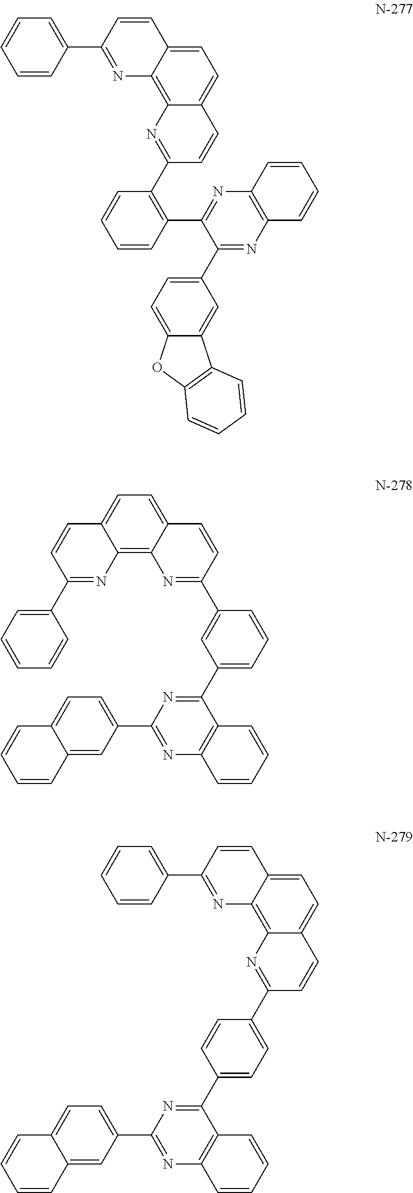 [SMILES: c1ccc(-c2ccc3ccc4ccc(-c5ccccc5-c5nc6ccccc6nc5-c5ccc6oc7ccccc7c6c5)nc4c3n2)cc1 ; c1ccc(-c2ccc3ccc4ccc(-c5cccc(-c6nc(-c7ccc8ccccc8c7)nc7ccccc67)c5)nc4c3n2)cc1 ; c1ccc(-c2ccc3ccc4ccc(-c5ccc(-c6nc(-c7ccc8ccccc8c7)nc7ccccc67)cc5)nc4c3n2)cc1] 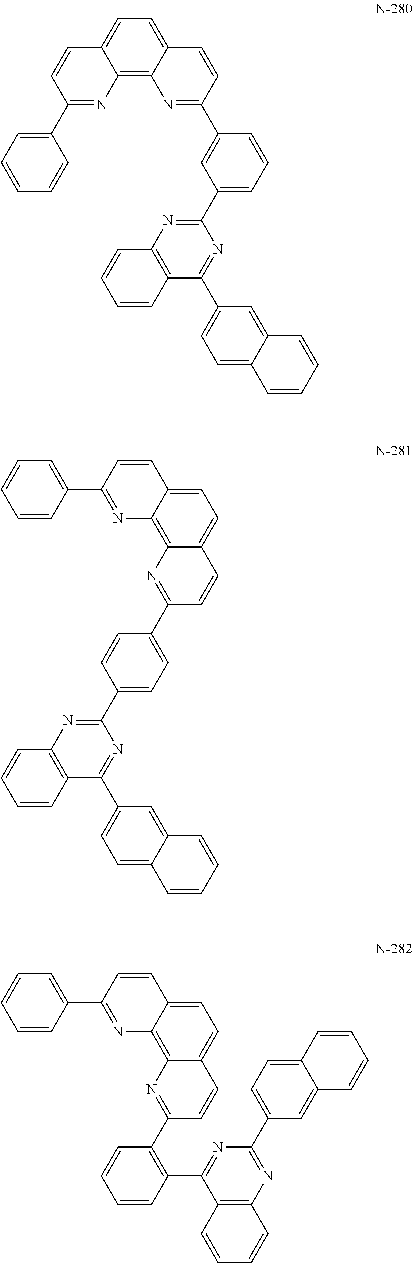 [SMILES: c1ccc(-c2ccc3ccc4ccc(-c5cccc(-c6nc(-c7ccc8ccccc8c7)c7ccccc7n6)c5)nc4c3n2)cc1 ; c1ccc(-c2ccc3ccc4ccc(-c5ccc(-c6nc(-c7ccc8ccccc8c7)c7ccccc7n6)cc5)nc4c3n2)cc1 ; c1ccc(-c2ccc3ccc4ccc(-c5ccccc5-c5nc(-c6ccc7ccccc7c6)nc6ccccc56)nc4c3n2)cc1] 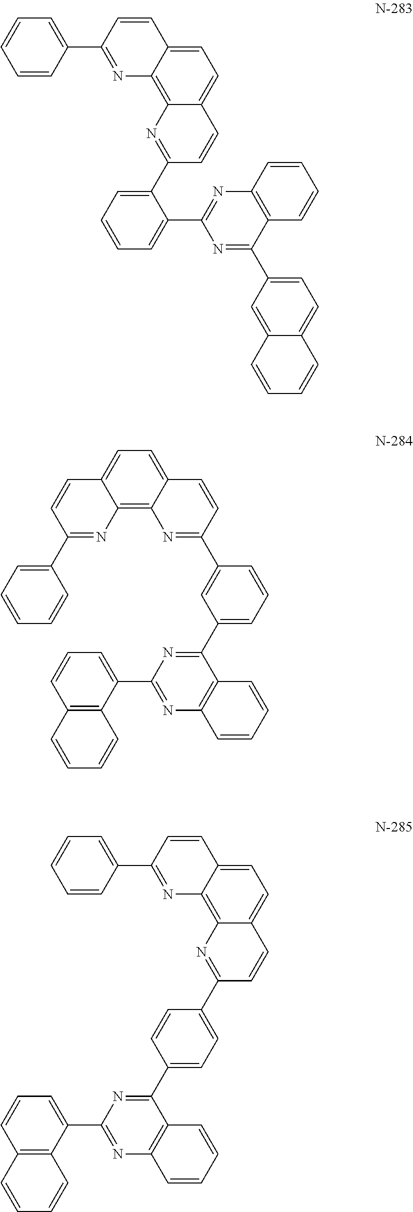 [SMILES: c1ccc(-c2ccc3ccc4ccc(-c5ccccc5-c5nc(-c6ccc7ccccc7c6)c6ccccc6n5)nc4c3n2)cc1 ; c1ccc(-c2ccc3ccc4ccc(-c5cccc(-c6nc(-c7cccc8ccccc78)nc7ccccc67)c5)nc4c3n2)cc1 ; c1ccc(-c2ccc3ccc4ccc(-c5ccc(-c6nc(-c7cccc8ccccc78)nc7ccccc67)cc5)nc4c3n2)cc1] 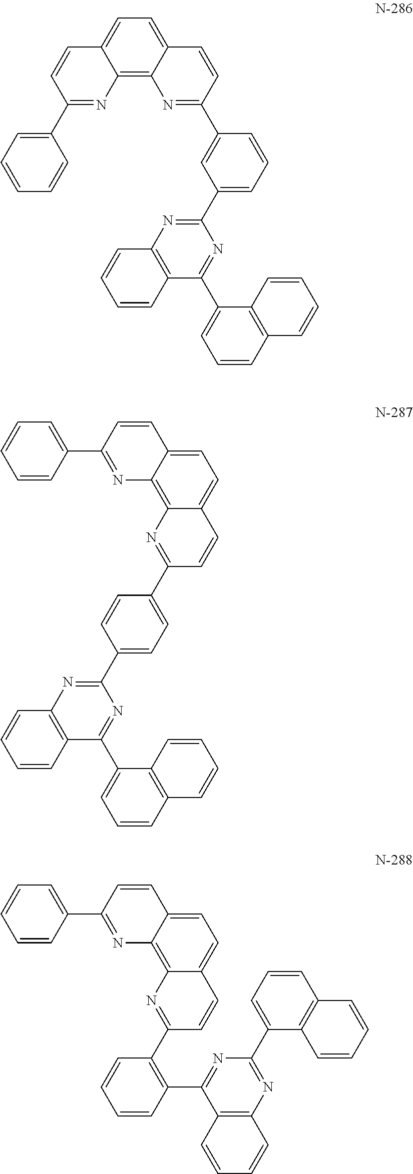 [SMILES: c1ccc(-c2ccc3ccc4ccc(-c5cccc(-c6nc(-c7cccc8ccccc78)c7ccccc7n6)c5)nc4c3n2)cc1 ; c1ccc(-c2ccc3ccc4ccc(-c5ccc(-c6nc(-c7cccc8ccccc78)c7ccccc7n6)cc5)nc4c3n2)cc1 ; c1ccc(-c2ccc3ccc4ccc(-c5ccccc5-c5nc(-c6cccc7ccccc67)nc6ccccc56)nc4c3n2)cc1] 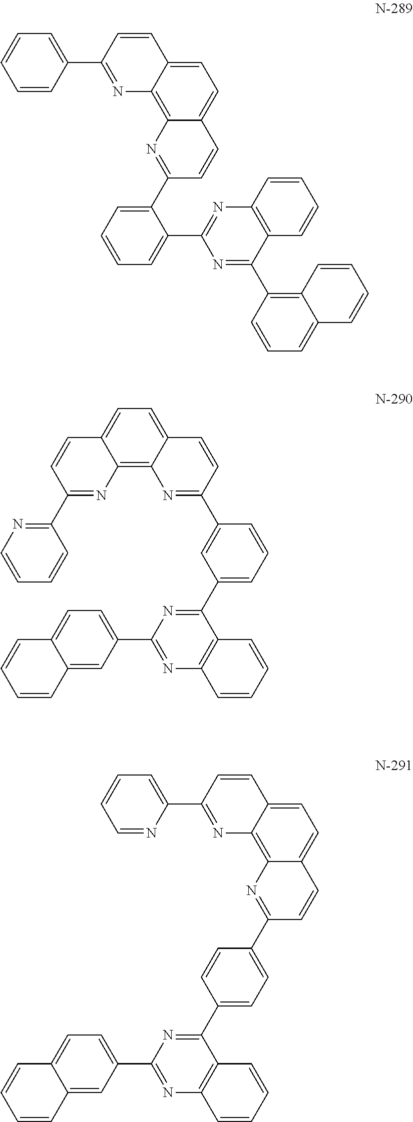 [SMILES: c1ccc(-c2ccc3ccc4ccc(-c5ccccc5-c5nc(-c6cccc7ccccc67)c6ccccc6n5)nc4c3n2)cc1 ; c1ccc(-c2ccc3ccc4ccc(-c5cccc(-c6nc(-c7ccc8ccccc8c7)nc7ccccc67)c5)nc4c3n2)nc1 ; c1ccc(-c2ccc3ccc4ccc(-c5ccc(-c6nc(-c7ccc8ccccc8c7)nc7ccccc67)cc5)nc4c3n2)nc1] 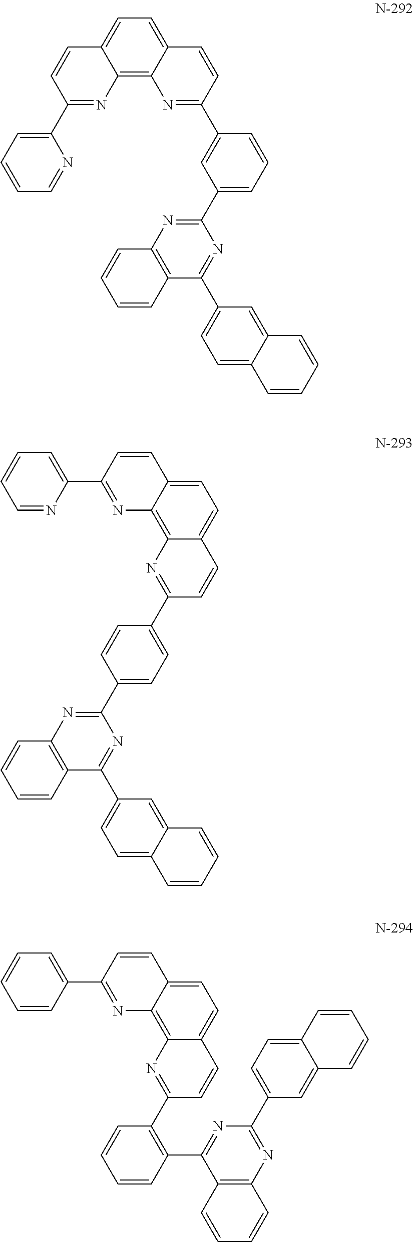 [SMILES: c1ccc(-c2ccc3ccc4ccc(-c5cccc(-c6nc(-c7ccc8ccccc8c7)c7ccccc7n6)c5)nc4c3n2)nc1 ; c1ccc(-c2ccc3ccc4ccc(-c5ccc(-c6nc(-c7ccc8ccccc8c7)c7ccccc7n6)cc5)nc4c3n2)nc1 ; c1ccc(-c2ccc3ccc4ccc(-c5ccccc5-c5nc(-c6ccc7ccccc7c6)nc6ccccc56)nc4c3n2)cc1] 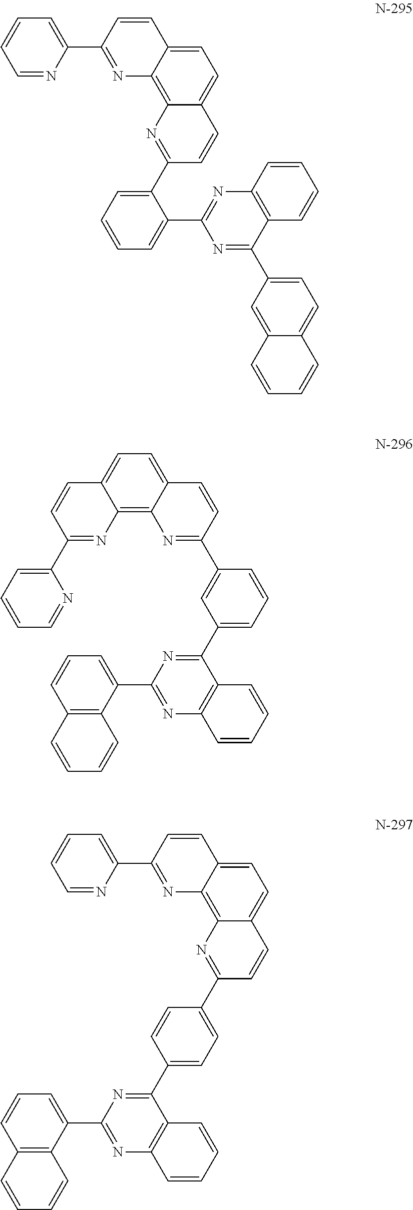 [SMILES: c1ccc(-c2ccc3ccc4ccc(-c5ccccc5-c5nc(-c6ccc7ccccc7c6)c6ccccc6n5)nc4c3n2)nc1 ; c1ccc(-c2ccc3ccc4ccc(-c5cccc(-c6nc(-c7cccc8ccccc78)nc7ccccc67)c5)nc4c3n2)nc1 ; c1ccc(-c2ccc3ccc4ccc(-c5ccc(-c6nc(-c7cccc8ccccc78)nc7ccccc67)cc5)nc4c3n2)nc1] 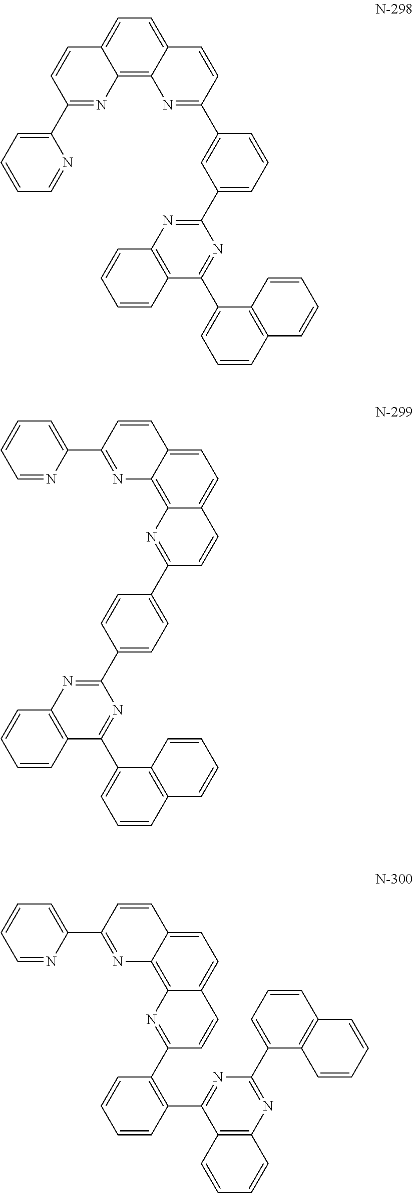 [SMILES: c1ccc(-c2ccc3ccc4ccc(-c5cccc(-c6nc(-c7cccc8ccccc78)c7ccccc7n6)c5)nc4c3n2)nc1 ; c1ccc(-c2ccc3ccc4ccc(-c5ccc(-c6nc(-c7cccc8ccccc78)c7ccccc7n6)cc5)nc4c3n2)nc1 ; c1ccc(-c2ccc3ccc4ccc(-c5ccccc5-c5nc(-c6cccc7ccccc67)nc6ccccc56)nc4c3n2)nc1] 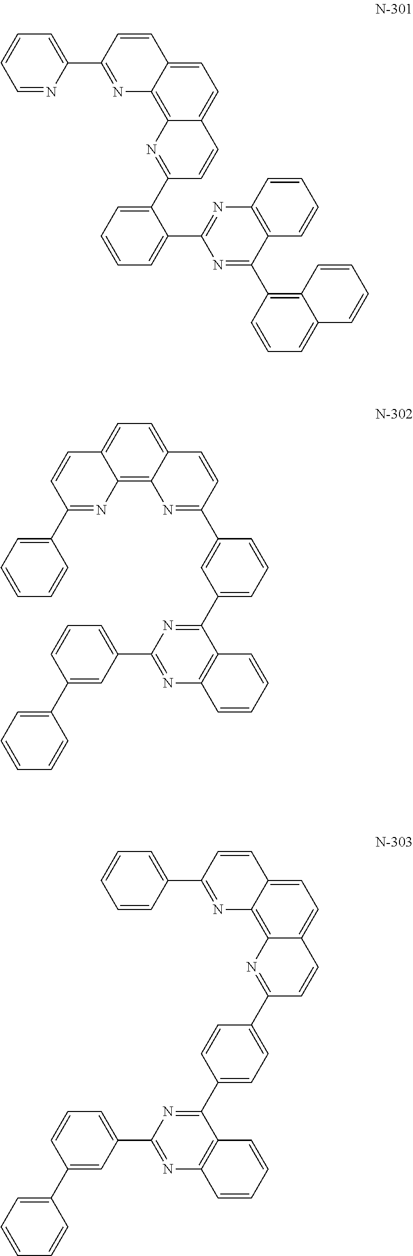 [SMILES: c1ccc(-c2ccc3ccc4ccc(-c5ccccc5-c5nc(-c6cccc7ccccc67)c6ccccc6n5)nc4c3n2)nc1 ; c1ccc(-c2cccc(-c3nc(-c4cccc(-c5ccc6ccc7ccc(-c8ccccc8)nc7c6n5)c4)c4ccccc4n3)c2)cc1 ; c1ccc(-c2cccc(-c3nc(-c4ccc(-c5ccc6ccc7ccc(-c8ccccc8)nc7c6n5)cc4)c4ccccc4n3)c2)cc1] 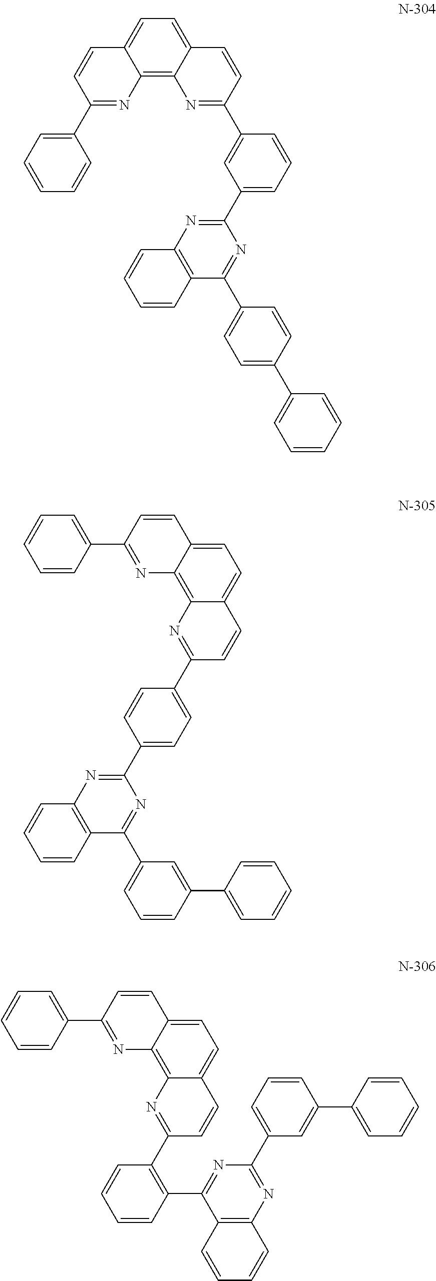 [SMILES: c1ccc(-c2ccc(-c3nc(-c4cccc(-c5ccc6ccc7ccc(-c8ccccc8)nc7c6n5)c4)nc4ccccc34)cc2)cc1 ; c1ccc(-c2cccc(-c3nc(-c4ccc(-c5ccc6ccc7ccc(-c8ccccc8)nc7c6n5)cc4)nc4ccccc34)c2)cc1 ; c1ccc(-c2cccc(-c3nc(-c4ccccc4-c4ccc5ccc6ccc(-c7ccccc7)nc6c5n4)c4ccccc4n3)c2)cc1] 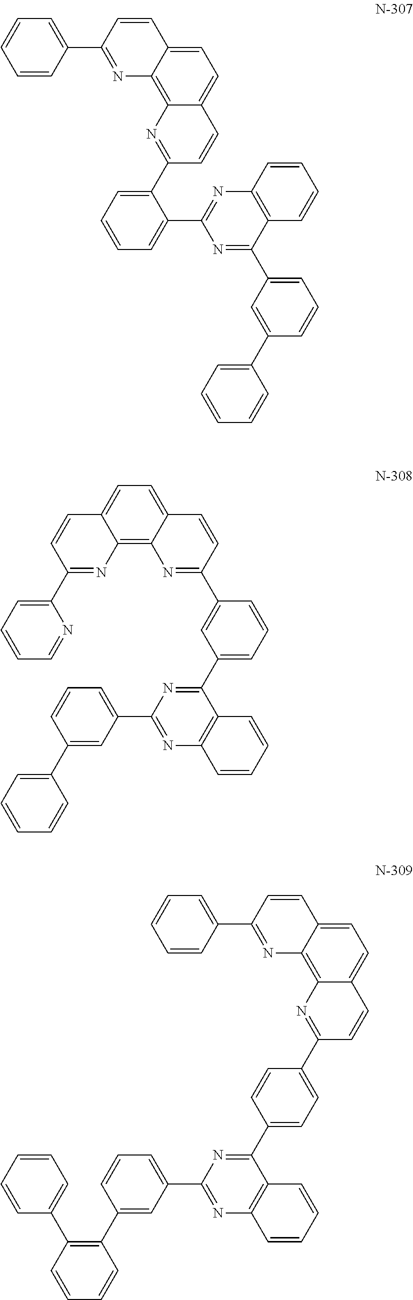 [SMILES: c1ccc(-c2cccc(-c3nc(-c4ccccc4-c4ccc5ccc6ccc(-c7ccccc7)nc6c5n4)nc4ccccc34)c2)cc1 ; c1ccc(-c2cccc(-c3nc(-c4cccc(-c5ccc6ccc7ccc(-c8ccccn8)nc7c6n5)c4)c4ccccc4n3)c2)cc1 ; c1ccc(-c2ccc3ccc4ccc(-c5ccc(-c6nc(-c7cccc(-c8ccccc8-c8ccccc8)c7)nc7ccccc67)cc5)nc4c3n2)cc1] 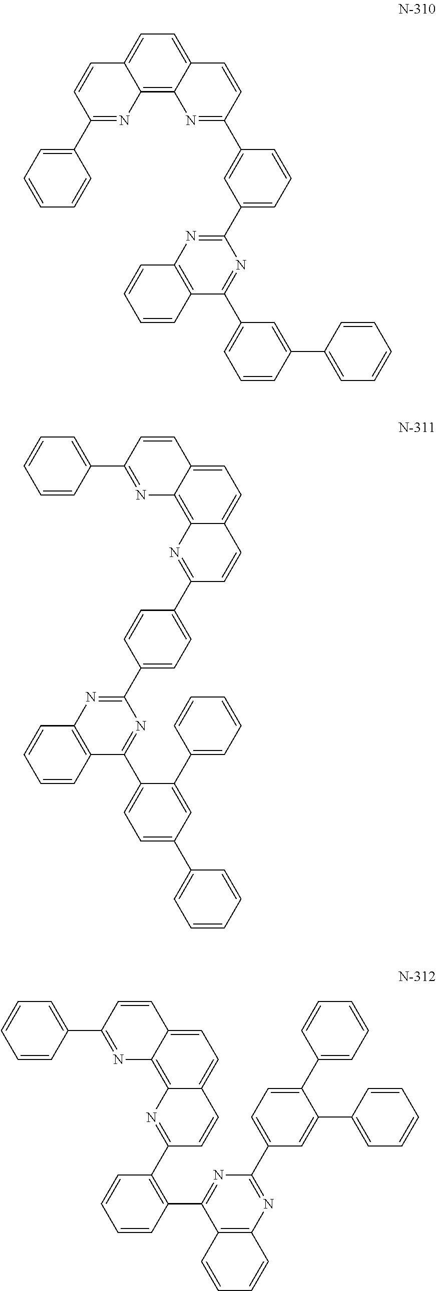 [SMILES: c1ccc(-c2cccc(-c3nc(-c4cccc(-c5ccc6ccc7ccc(-c8ccccc8)nc7c6n5)c4)nc4ccccc34)c2)cc1 ; c1ccc(-c2ccc(-c3nc(-c4ccc(-c5ccc6ccc7ccc(-c8ccccc8)nc7c6n5)cc4)nc4ccccc34)c(-c3ccccc3)c2)cc1 ; c1ccc(-c2ccc3ccc4ccc(-c5ccccc5-c5nc(-c6ccc(-c7ccccc7)c(-c7ccccc7)c6)nc6ccccc56)nc4c3n2)cc1] 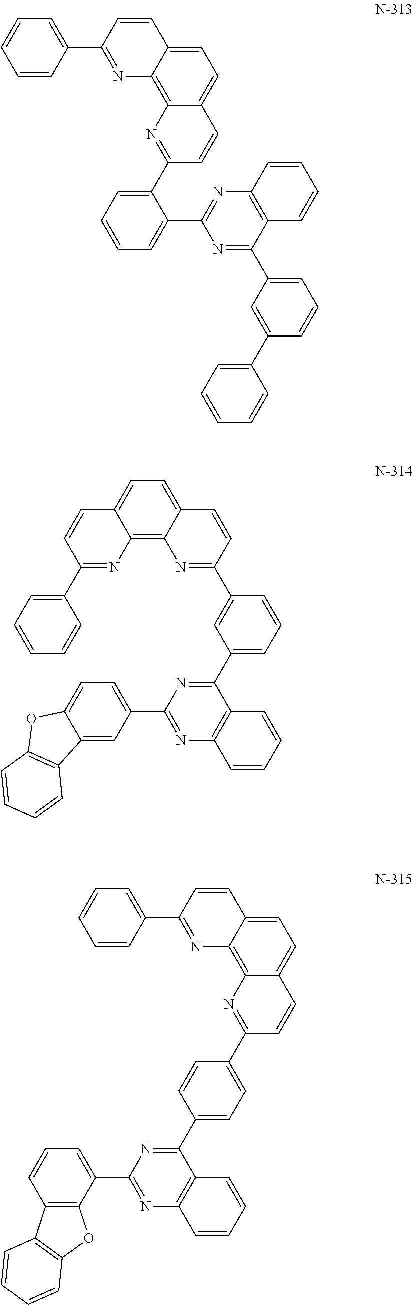 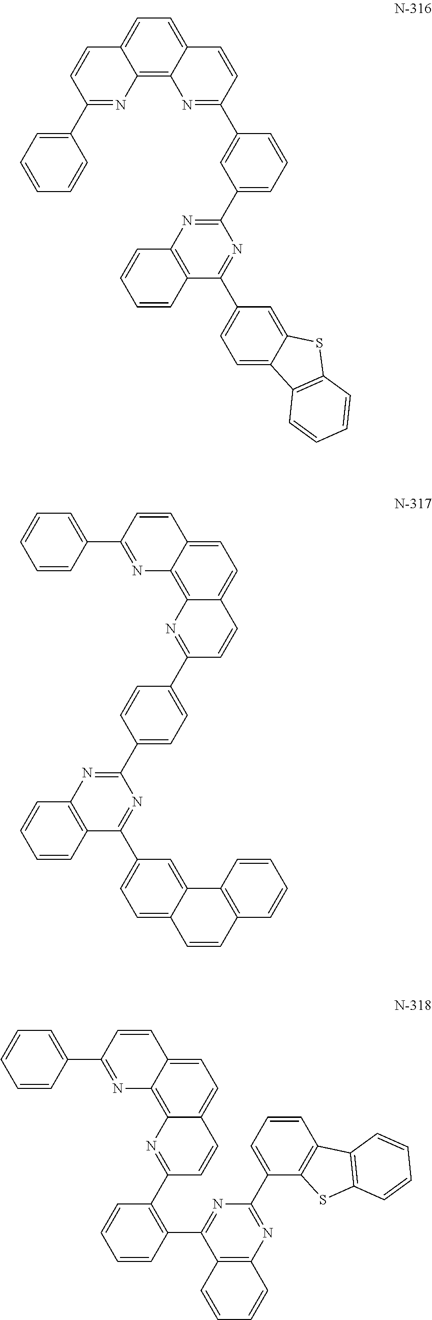 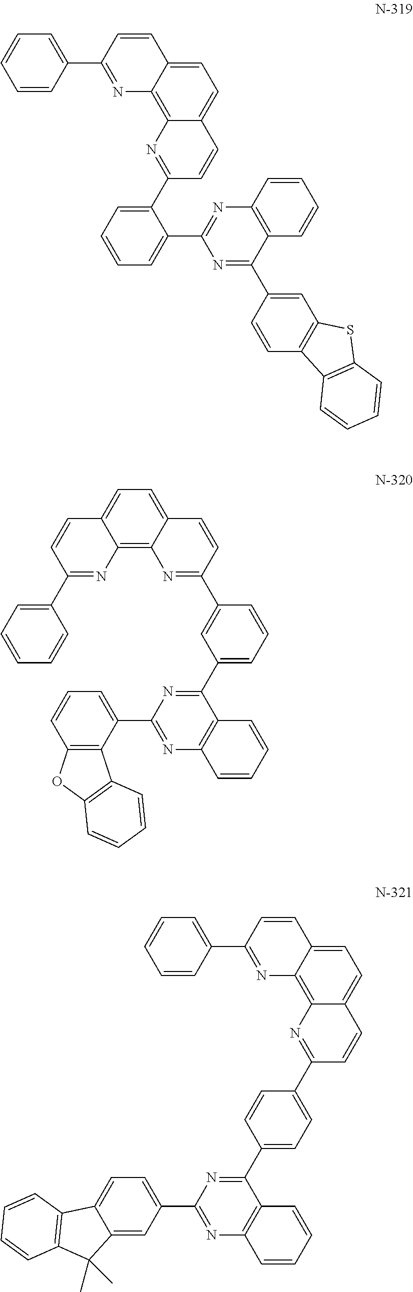 [SMILES: c1ccc(-c2ccc3ccc4ccc(-c5ccccc5-c5nc(-c6ccc7c(c6)sc6ccccc67)c6ccccc6n5)nc4c3n2)cc1 ; c1ccc(-c2ccc3ccc4ccc(-c5cccc(-c6nc(-c7cccc8oc9ccccc9c78)nc7ccccc67)c5)nc4c3n2)cc1 ; CC1(C)c2ccccc2-c2ccc(-c3nc(-c4ccc(-c5ccc6ccc7ccc(-c8ccccc8)nc7c6n5)cc4)c4ccccc4n3)cc21] 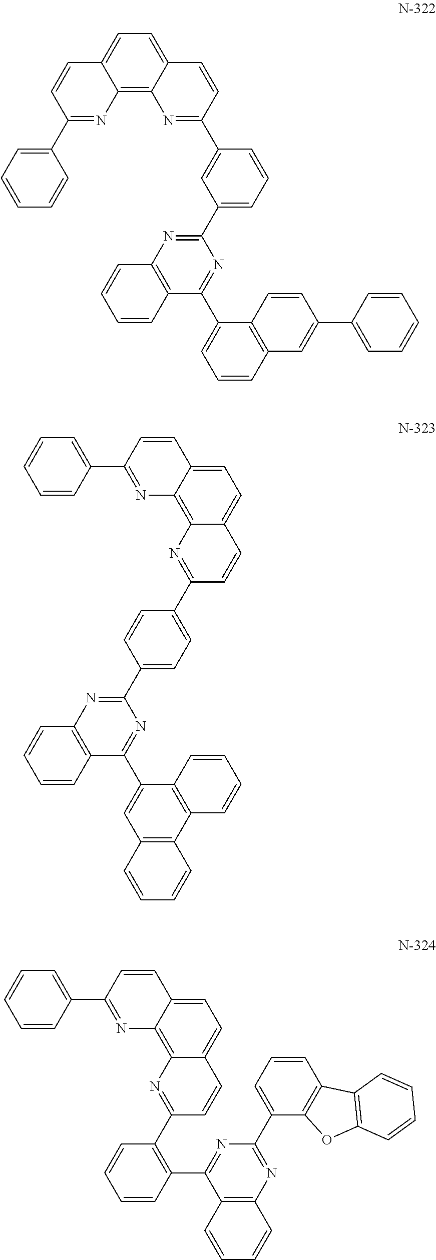 [SMILES: c1ccc(-c2ccc3c(-c4nc(-c5cccc(-c6ccc7ccc8ccc(-c9ccccc9)nc8c7n6)c5)nc5ccccc45)cccc3c2)cc1 ; c1ccc(-c2ccc3ccc4ccc(-c5ccc(-c6nc(-c7cc8ccccc8c8ccccc78)c7ccccc7n6)cc5)nc4c3n2)cc1 ; c1ccc(-c2ccc3ccc4ccc(-c5ccccc5-c5nc(-c6cccc7c6oc6ccccc67)nc6ccccc56)nc4c3n2)cc1] 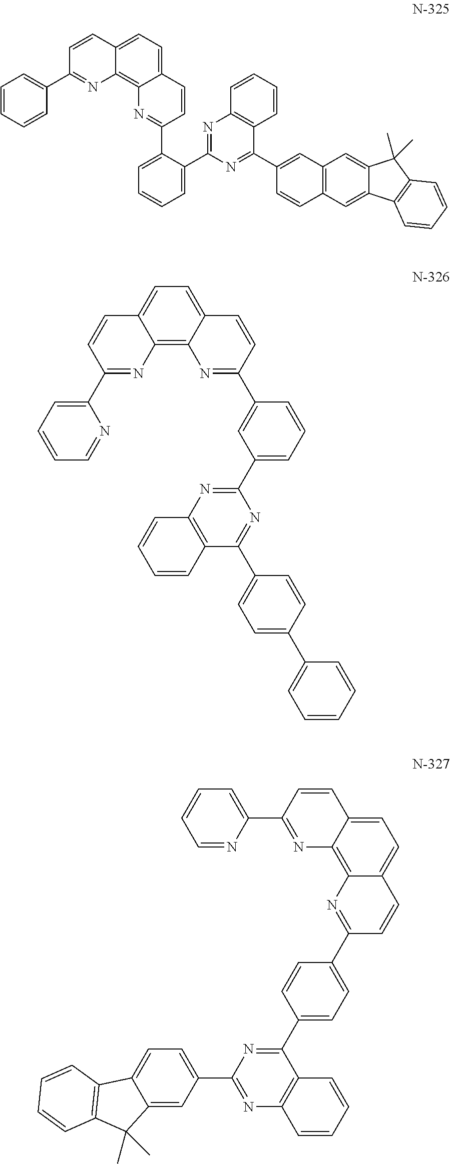 [SMILES: CC1(C)c2ccccc2-c2cc3ccc(-c4nc(-c5ccccc5-c5ccc6ccc7ccc(-c8ccccc8)nc7c6n5)nc5ccccc45)cc3cc21 ; c1ccc(-c2ccc(-c3nc(-c4cccc(-c5ccc6ccc7ccc(-c8ccccn8)nc7c6n5)c4)nc4ccccc34)cc2)cc1 ; CC1(C)c2ccccc2-c2ccc(-c3nc(-c4ccc(-c5ccc6ccc7ccc(-c8ccccn8)nc7c6n5)cc4)c4ccccc4n3)cc21] 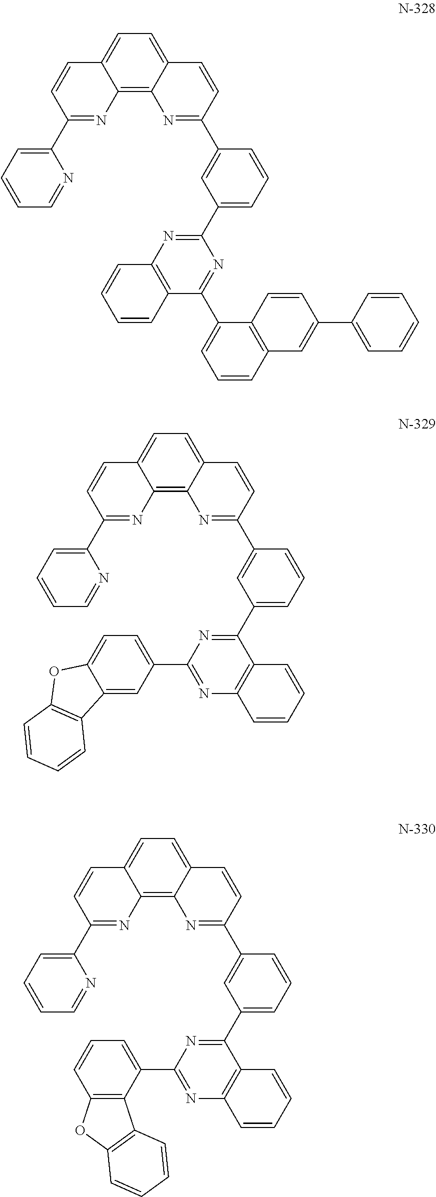 [SMILES: c1ccc(-c2ccc3c(-c4nc(-c5cccc(-c6ccc7ccc8ccc(-c9ccccn9)nc8c7n6)c5)nc5ccccc45)cccc3c2)cc1 ; c1ccc(-c2ccc3ccc4ccc(-c5cccc(-c6nc(-c7ccc8oc9ccccc9c8c7)nc7ccccc67)c5)nc4c3n2)nc1 ; c1ccc(-c2ccc3ccc4ccc(-c5cccc(-c6nc(-c7cccc8oc9ccccc9c78)nc7ccccc67)c5)nc4c3n2)nc1] 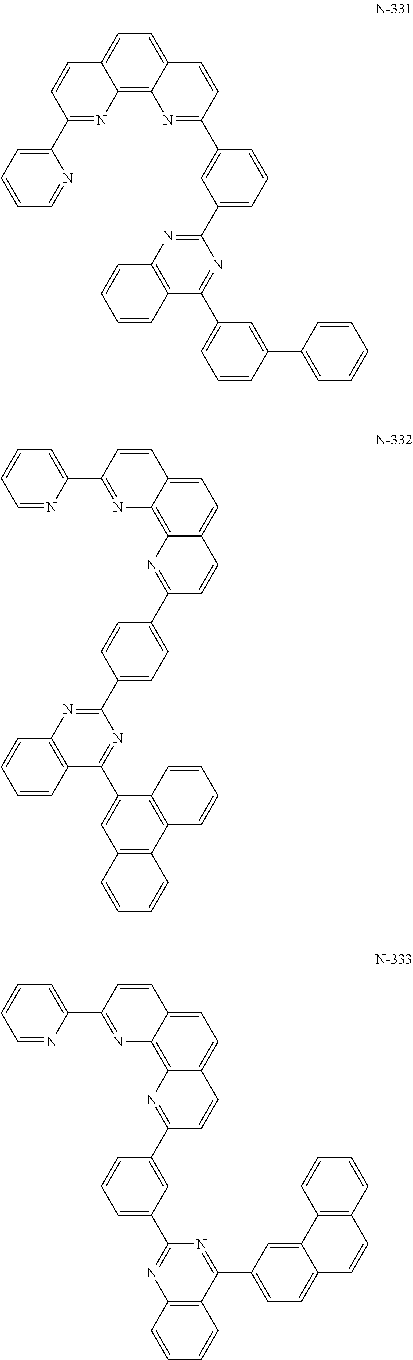 [SMILES: c1ccc(-c2cccc(-c3nc(-c4cccc(-c5ccc6ccc7ccc(-c8ccccn8)nc7c6n5)c4)nc4ccccc34)c2)cc1 ; c1ccc(-c2ccc3ccc4ccc(-c5ccc(-c6nc(-c7cc8ccccc8c8ccccc78)c7ccccc7n6)cc5)nc4c3n2)nc1 ; c1ccc(-c2ccc3ccc4ccc(-c5cccc(-c6nc(-c7ccc8ccc9ccccc9c8c7)c7ccccc7n6)c5)nc4c3n2)nc1] 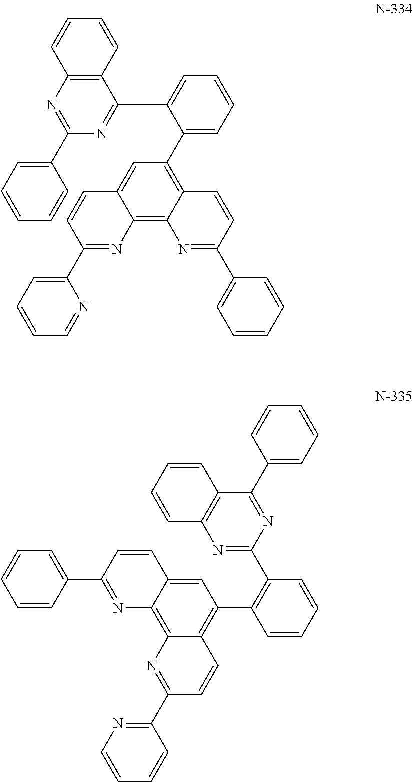 [SMILES: c1ccc(-c2ccc3c(-c4ccccc4-c4nc(-c5ccccc5)nc5ccccc45)cc4ccc(-c5ccccn5)nc4c3n2)cc1 ; c1ccc(-c2ccc3cc(-c4ccccc4-c4nc(-c5ccccc5)c5ccccc5n4)c4ccc(-c5ccccn5)nc4c3n2)cc1] 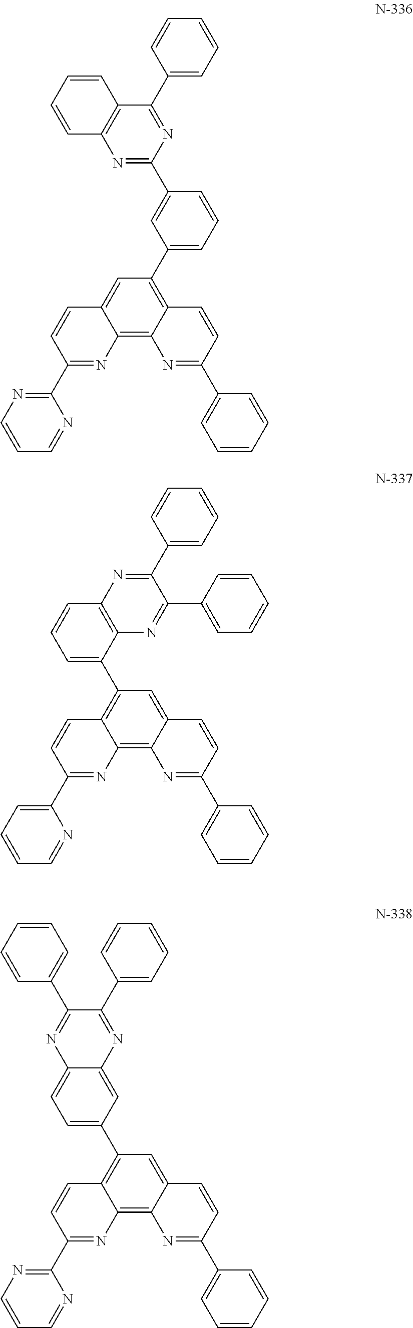 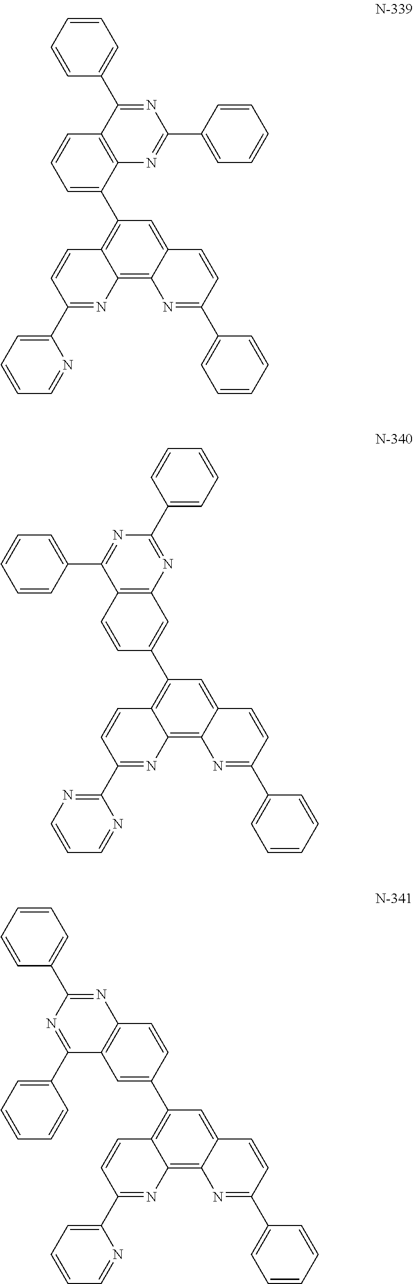 [SMILES: c1ccc(-c2ccc3cc(-c4cccc5c(-c6ccccc6)nc(-c6ccccc6)nc45)c4ccc(-c5ccccn5)nc4c3n2)cc1 ; c1ccc(-c2ccc3cc(-c4ccc5c(-c6ccccc6)nc(-c6ccccc6)nc5c4)c4ccc(-c5ncccn5)nc4c3n2)cc1 ; c1ccc(-c2ccc3cc(-c4ccc5nc(-c6ccccc6)nc(-c6ccccc6)c5c4)c4ccc(-c5ccccn5)nc4c3n2)cc1] 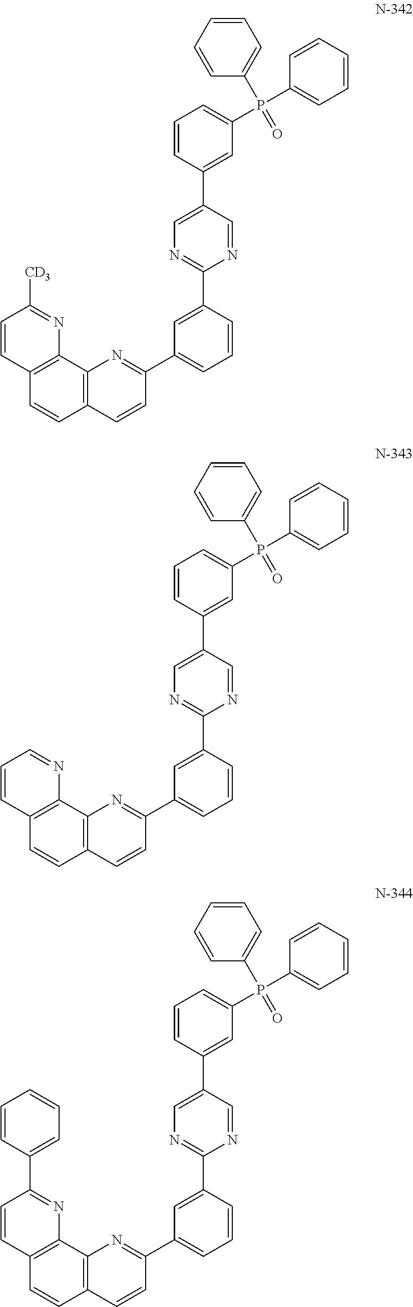 [SMILES: [2H]C([2H])([2H])c1ccc2ccc3ccc(-c4cccc(-c5ncc(-c6cccc(P(=O)(c7ccccc7)c7ccccc7)c6)cn5)c4)nc3c2n1 ; O=P(c1ccccc1)(c1ccccc1)c1cccc(-c2cnc(-c3cccc(-c4ccc5ccc6cccnc6c5n4)c3)nc2)c1 ; O=P(c1ccccc1)(c1ccccc1)c1cccc(-c2cnc(-c3cccc(-c4ccc5ccc6ccc(-c7ccccc7)nc6c5n4)c3)nc2)c1] 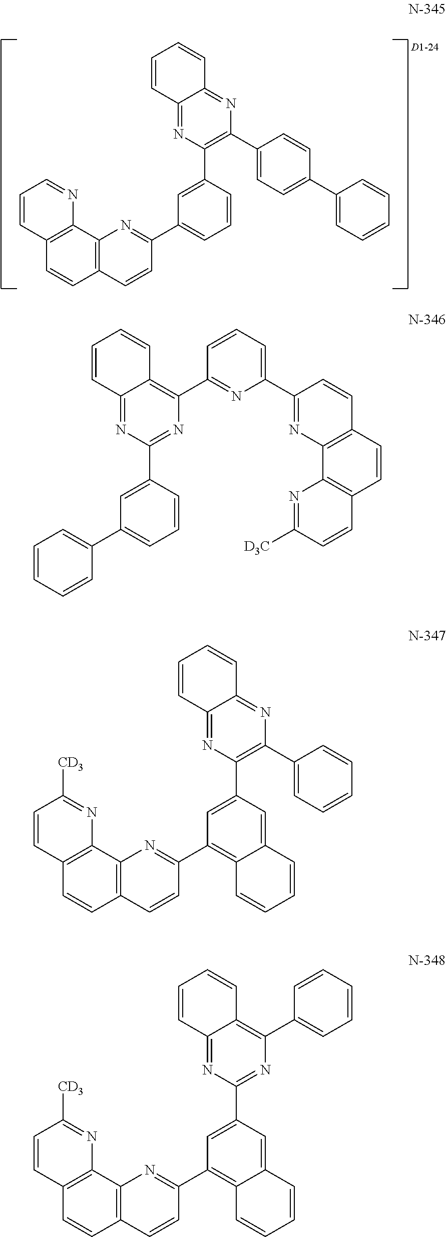 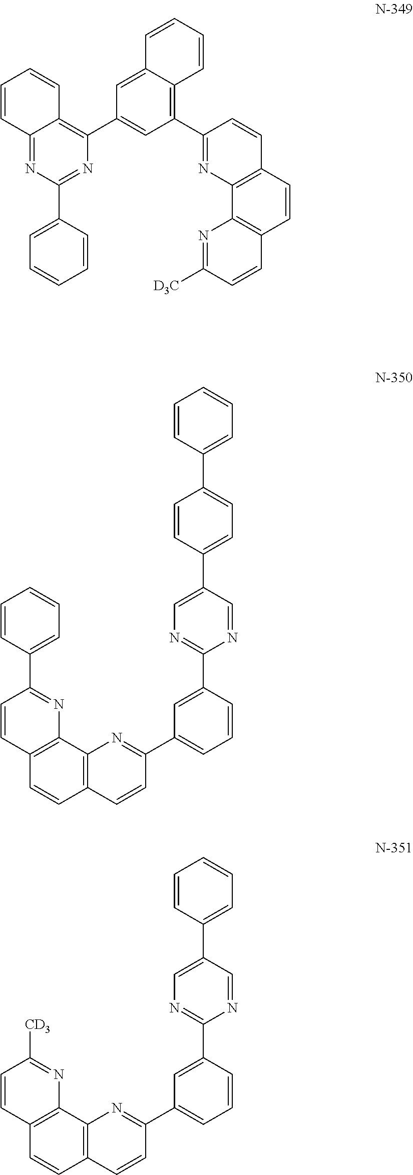 [SMILES: [2H]C([2H])([2H])c1ccc2ccc3ccc(-c4cc(-c5nc(-c6ccccc6)nc6ccccc56)cc5ccccc45)nc3c2n1 ; c1ccc(-c2ccc(-c3cnc(-c4cccc(-c5ccc6ccc7ccc(-c8ccccc8)nc7c6n5)c4)nc3)cc2)cc1 ; [2H]C([2H])([2H])c1ccc2ccc3ccc(-c4cccc(-c5ncc(-c6ccccc6)cn5)c4)nc3c2n1] 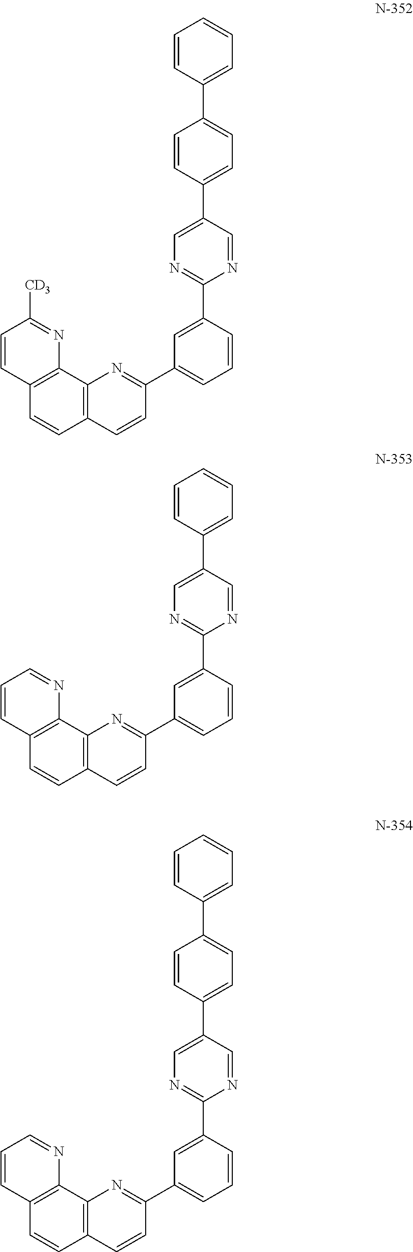 [SMILES: [2H]C([2H])([2H])c1ccc2ccc3ccc(-c4cccc(-c5ncc(-c6ccc(-c7ccccc7)cc6)cn5)c4)nc3c2n1 ; c1ccc(-c2cnc(-c3cccc(-c4ccc5ccc6cccnc6c5n4)c3)nc2)cc1 ; c1ccc(-c2ccc(-c3cnc(-c4cccc(-c5ccc6ccc7cccnc7c6n5)c4)nc3)cc2)cc1] 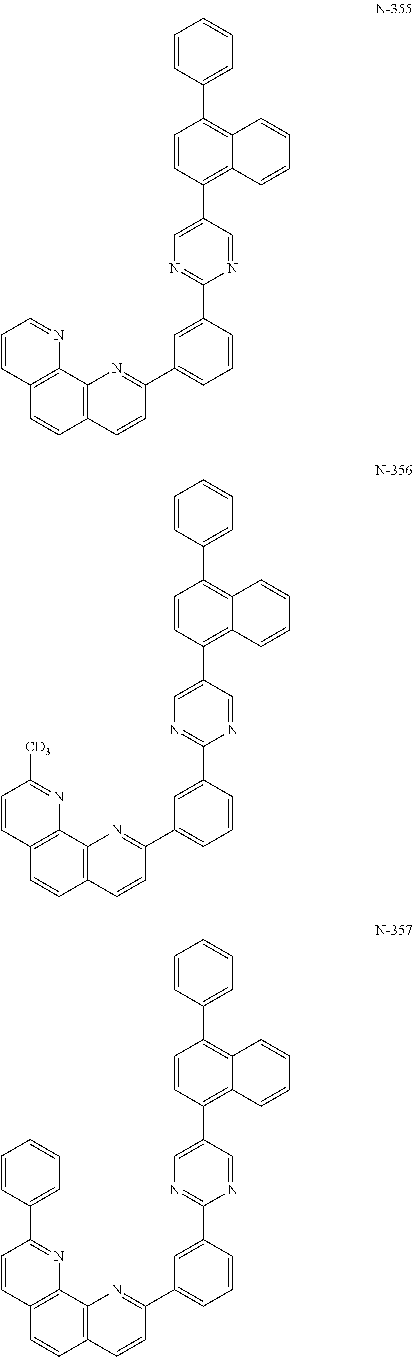 [SMILES: c1ccc(-c2ccc(-c3cnc(-c4cccc(-c5ccc6ccc7cccnc7c6n5)c4)nc3)c3ccccc23)cc1 ; [2H]C([2H])([2H])c1ccc2ccc3ccc(-c4cccc(-c5ncc(-c6ccc(-c7ccccc7)c7ccccc67)cn5)c4)nc3c2n1 ; c1ccc(-c2ccc3ccc4ccc(-c5cccc(-c6ncc(-c7ccc(-c8ccccc8)c8ccccc78)cn6)c5)nc4c3n2)cc1] 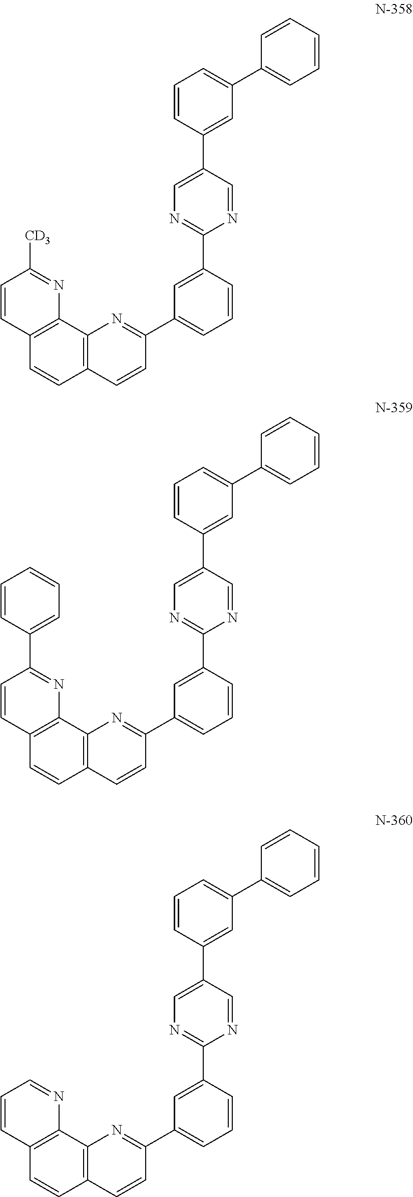 [SMILES: [2H]C([2H])([2H])c1ccc2ccc3ccc(-c4cccc(-c5ncc(-c6cccc(-c7ccccc7)c6)cn5)c4)nc3c2n1 ; c1ccc(-c2cccc(-c3cnc(-c4cccc(-c5ccc6ccc7ccc(-c8ccccc8)nc7c6n5)c4)nc3)c2)cc1 ; c1ccc(-c2cccc(-c3cnc(-c4cccc(-c5ccc6ccc7cccnc7c6n5)c4)nc3)c2)cc1] 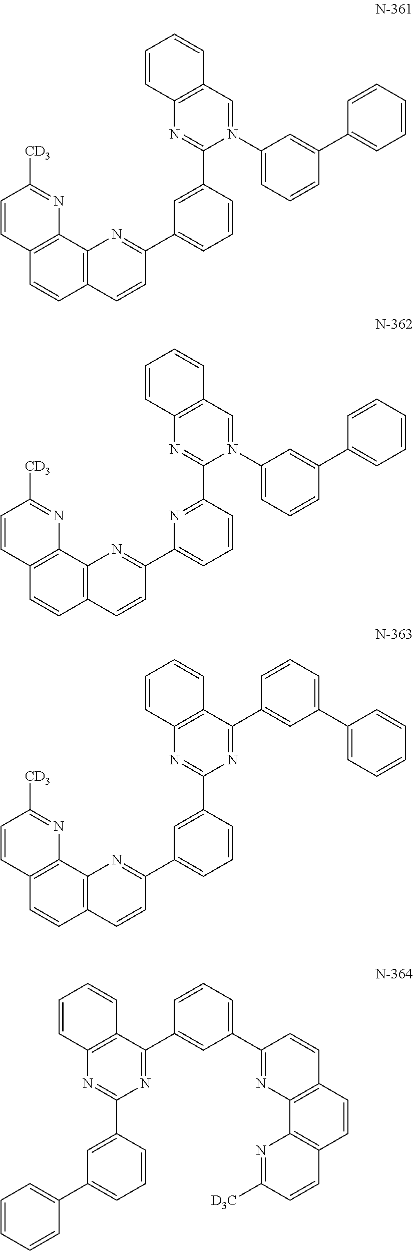 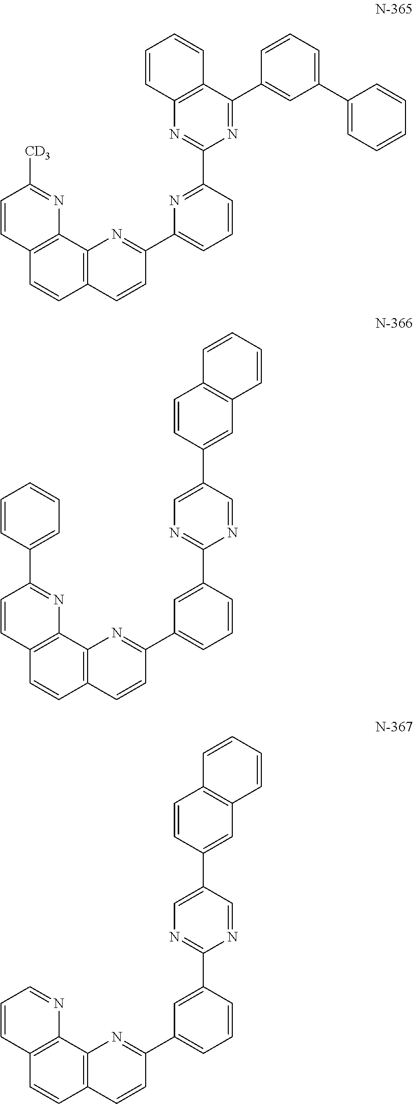 [SMILES: [2H]C([2H])([2H])c1ccc2ccc3ccc(-c4cccc(-c5nc(-c6cccc(-c7ccccc7)c6)c6ccccc6n5)n4)nc3c2n1 ; c1ccc(-c2ccc3ccc4ccc(-c5cccc(-c6ncc(-c7ccc8ccccc8c7)cn6)c5)nc4c3n2)cc1 ; c1cc(-c2ccc3ccc4cccnc4c3n2)cc(-c2ncc(-c3ccc4ccccc4c3)cn2)c1] 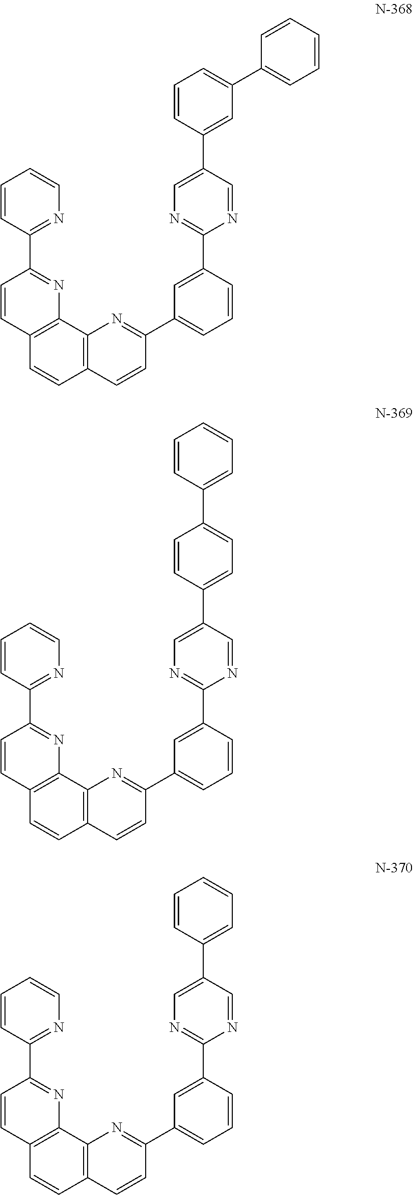 [SMILES: c1ccc(-c2cccc(-c3cnc(-c4cccc(-c5ccc6ccc7ccc(-c8ccccn8)nc7c6n5)c4)nc3)c2)cc1 ; c1ccc(-c2ccc(-c3cnc(-c4cccc(-c5ccc6ccc7ccc(-c8ccccn8)nc7c6n5)c4)nc3)cc2)cc1 ; c1ccc(-c2cnc(-c3cccc(-c4ccc5ccc6ccc(-c7ccccn7)nc6c5n4)c3)nc2)cc1] 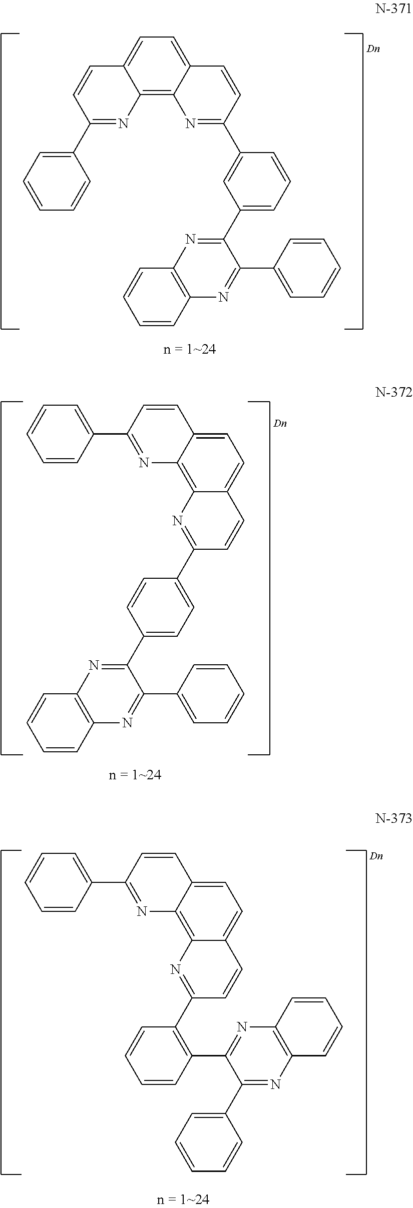 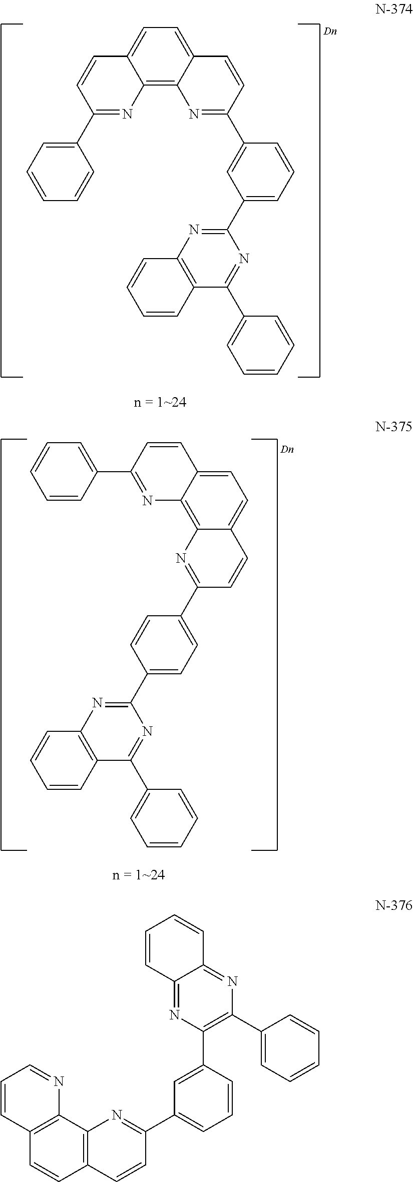 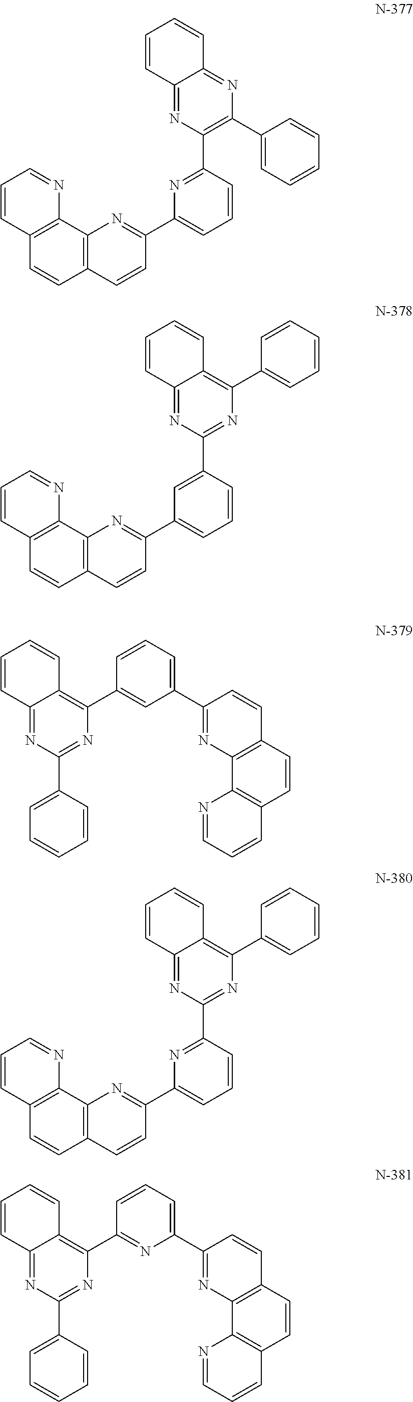 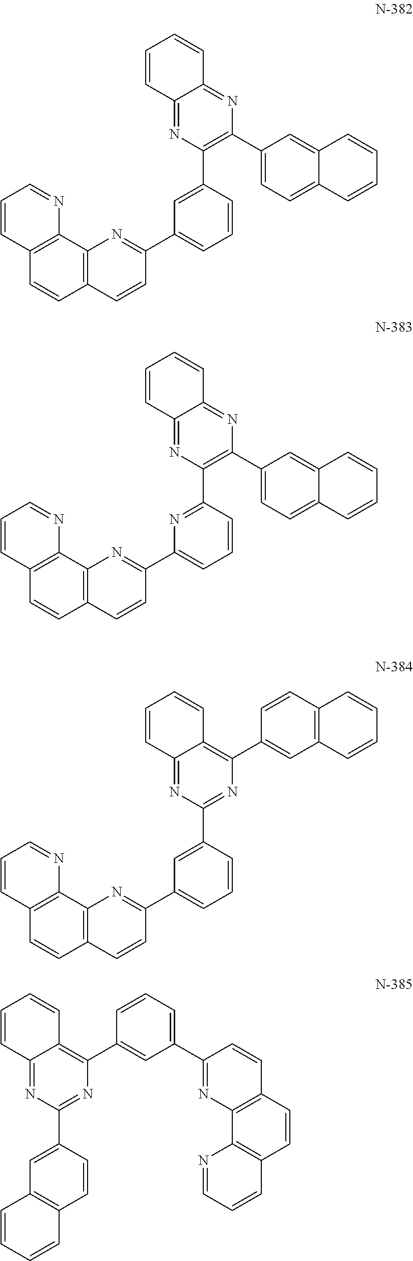 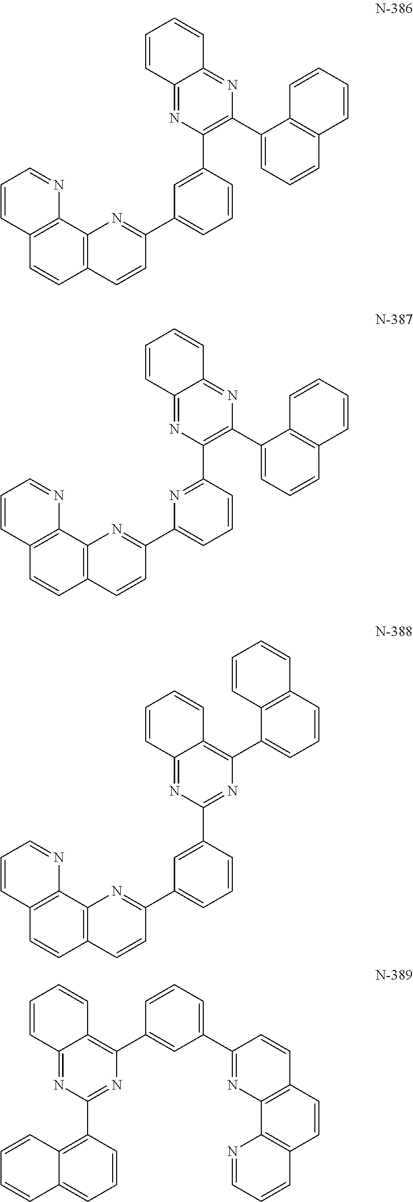 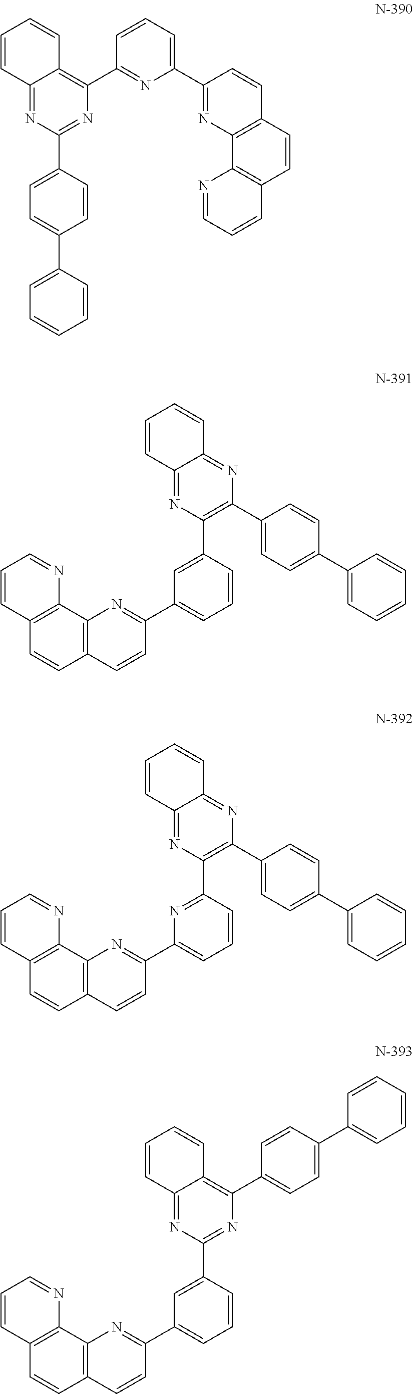 [SMILES: c1ccc(-c2ccc(-c3nc(-c4cccc(-c5ccc6ccc7cccnc7c6n5)n4)c4ccccc4n3)cc2)cc1 ; c1ccc(-c2ccc(-c3nc4ccccc4nc3-c3cccc(-c4ccc5ccc6cccnc6c5n4)c3)cc2)cc1 ; c1ccc(-c2ccc(-c3nc4ccccc4nc3-c3cccc(-c4ccc5ccc6cccnc6c5n4)n3)cc2)cc1 ; c1ccc(-c2ccc(-c3nc(-c4cccc(-c5ccc6ccc7cccnc7c6n5)c4)nc4ccccc34)cc2)cc1] 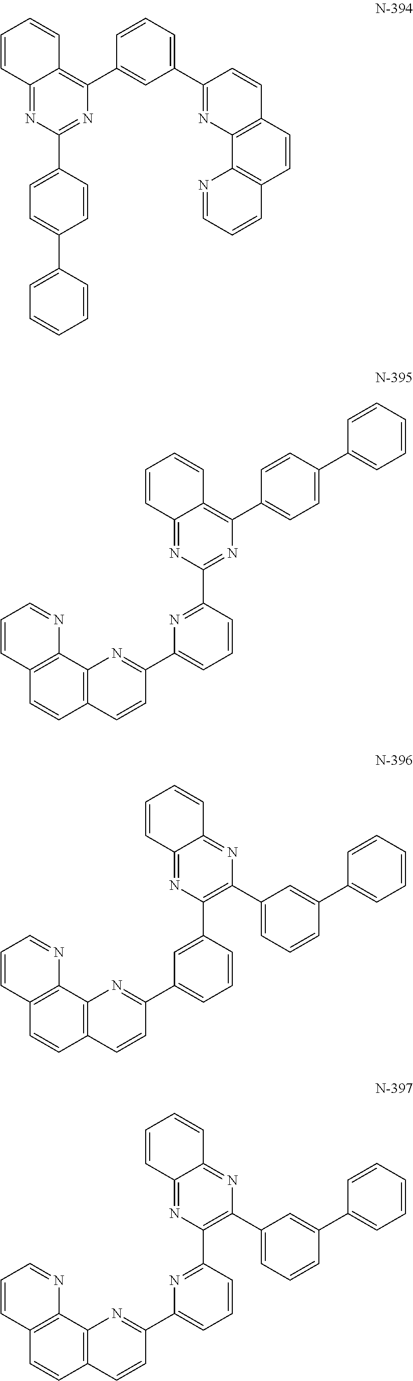 [SMILES: c1ccc(-c2ccc(-c3nc(-c4cccc(-c5ccc6ccc7cccnc7c6n5)c4)c4ccccc4n3)cc2)cc1 ; c1ccc(-c2ccc(-c3nc(-c4cccc(-c5ccc6ccc7cccnc7c6n5)n4)nc4ccccc34)cc2)cc1 ; c1ccc(-c2cccc(-c3nc4ccccc4nc3-c3cccc(-c4ccc5ccc6cccnc6c5n4)c3)c2)cc1 ; c1ccc(-c2cccc(-c3nc4ccccc4nc3-c3cccc(-c4ccc5ccc6cccnc6c5n4)n3)c2)cc1] 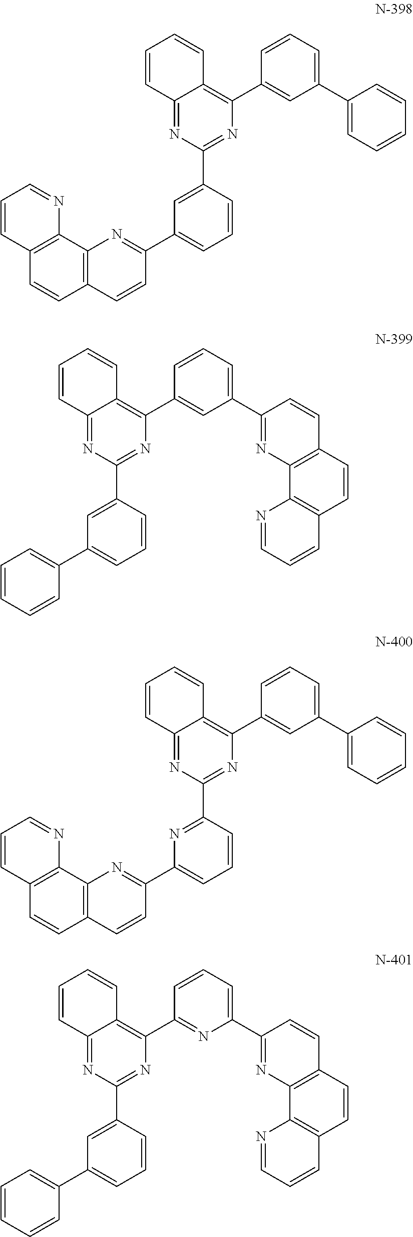 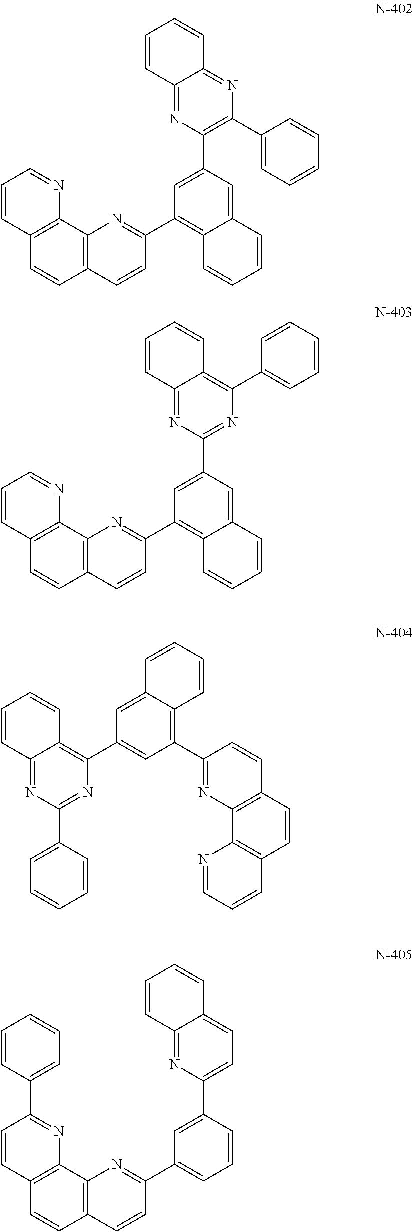 [SMILES: c1ccc(-c2nc3ccccc3nc2-c2cc(-c3ccc4ccc5cccnc5c4n3)c3ccccc3c2)cc1 ; c1ccc(-c2nc(-c3cc(-c4ccc5ccc6cccnc6c5n4)c4ccccc4c3)nc3ccccc23)cc1 ; c1ccc(-c2nc(-c3cc(-c4ccc5ccc6cccnc6c5n4)c4ccccc4c3)c3ccccc3n2)cc1 ; c1ccc(-c2ccc3ccc4ccc(-c5cccc(-c6ccc7ccccc7n6)c5)nc4c3n2)cc1] 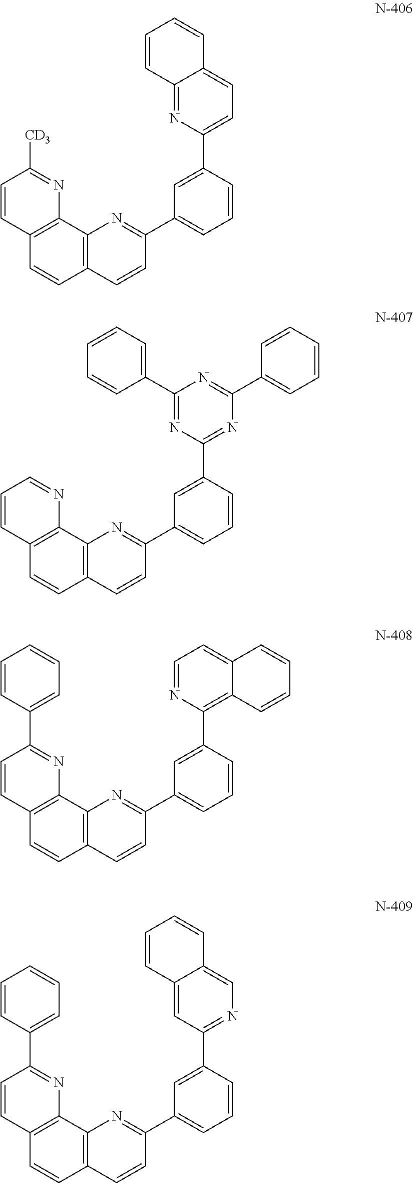 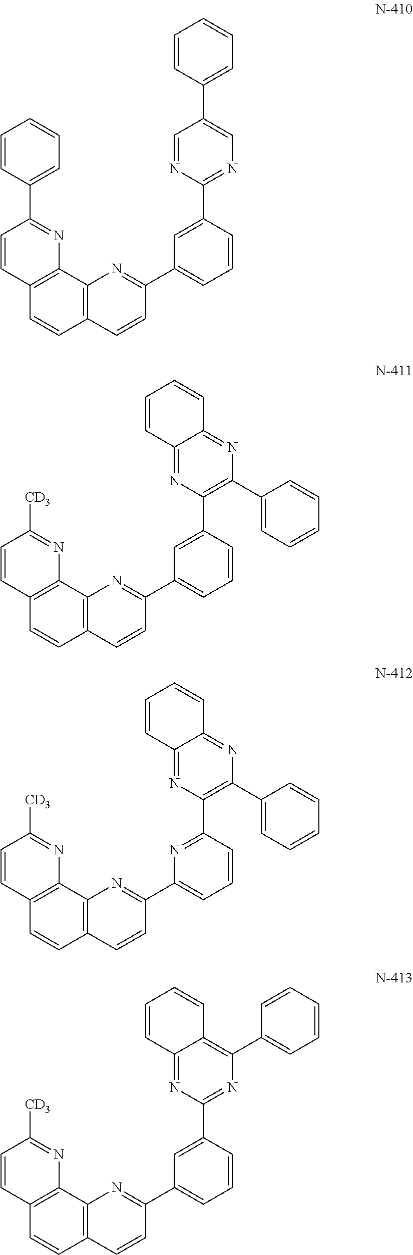 [SMILES: c1ccc(-c2cnc(-c3cccc(-c4ccc5ccc6ccc(-c7ccccc7)nc6c5n4)c3)nc2)cc1 ; [2H]C([2H])([2H])c1ccc2ccc3ccc(-c4cccc(-c5nc6ccccc6nc5-c5ccccc5)c4)nc3c2n1 ; [2H]C([2H])([2H])c1ccc2ccc3ccc(-c4cccc(-c5nc6ccccc6nc5-c5ccccc5)n4)nc3c2n1 ; [2H]C([2H])([2H])c1ccc2ccc3ccc(-c4cccc(-c5nc(-c6ccccc6)c6ccccc6n5)c4)nc3c2n1] 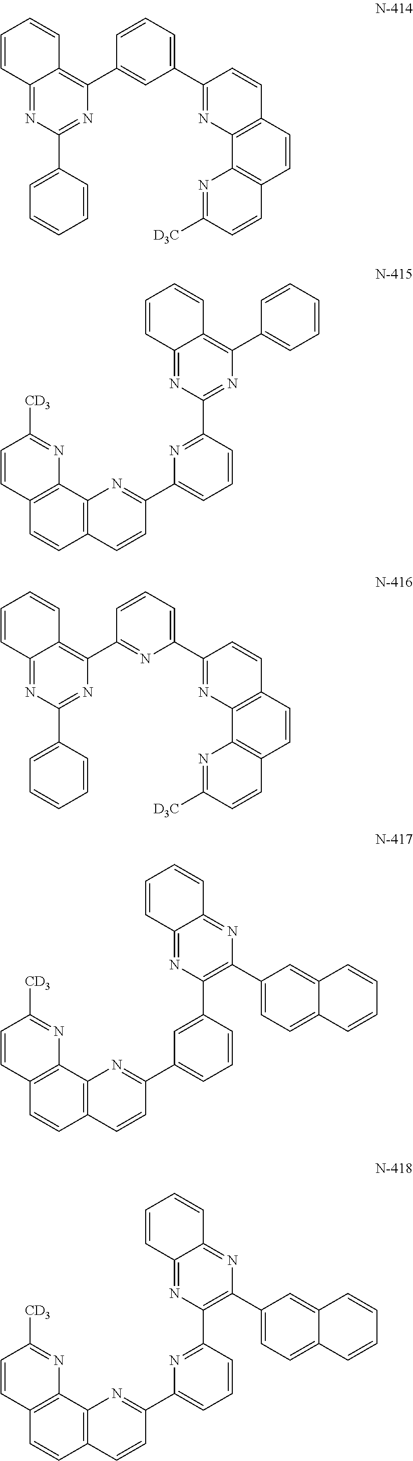 [SMILES: [2H]C([2H])([2H])c1ccc2ccc3ccc(-c4cccc(-c5nc(-c6ccccc6)nc6ccccc56)c4)nc3c2n1 ; [2H]C([2H])([2H])c1ccc2ccc3ccc(-c4cccc(-c5nc(-c6ccccc6)c6ccccc6n5)n4)nc3c2n1 ; [2H]C([2H])([2H])c1ccc2ccc3ccc(-c4cccc(-c5nc(-c6ccccc6)nc6ccccc56)n4)nc3c2n1 ; [2H]C([2H])([2H])c1ccc2ccc3ccc(-c4cccc(-c5nc6ccccc6nc5-c5ccc6ccccc6c5)c4)nc3c2n1 ; [2H]C([2H])([2H])c1ccc2ccc3ccc(-c4cccc(-c5nc6ccccc6nc5-c5ccc6ccccc6c5)n4)nc3c2n1] 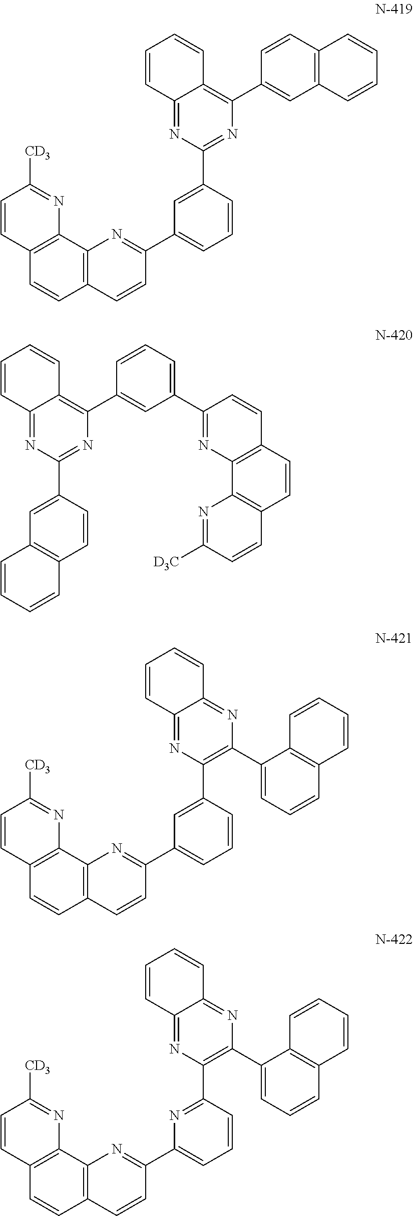 [SMILES: [2H]C([2H])([2H])c1ccc2ccc3ccc(-c4cccc(-c5nc(-c6ccc7ccccc7c6)c6ccccc6n5)c4)nc3c2n1 ; [2H]C([2H])([2H])c1ccc2ccc3ccc(-c4cccc(-c5nc(-c6ccc7ccccc7c6)nc6ccccc56)c4)nc3c2n1 ; [2H]C([2H])([2H])c1ccc2ccc3ccc(-c4cccc(-c5nc6ccccc6nc5-c5cccc6ccccc56)c4)nc3c2n1 ; [2H]C([2H])([2H])c1ccc2ccc3ccc(-c4cccc(-c5nc6ccccc6nc5-c5cccc6ccccc56)n4)nc3c2n1] 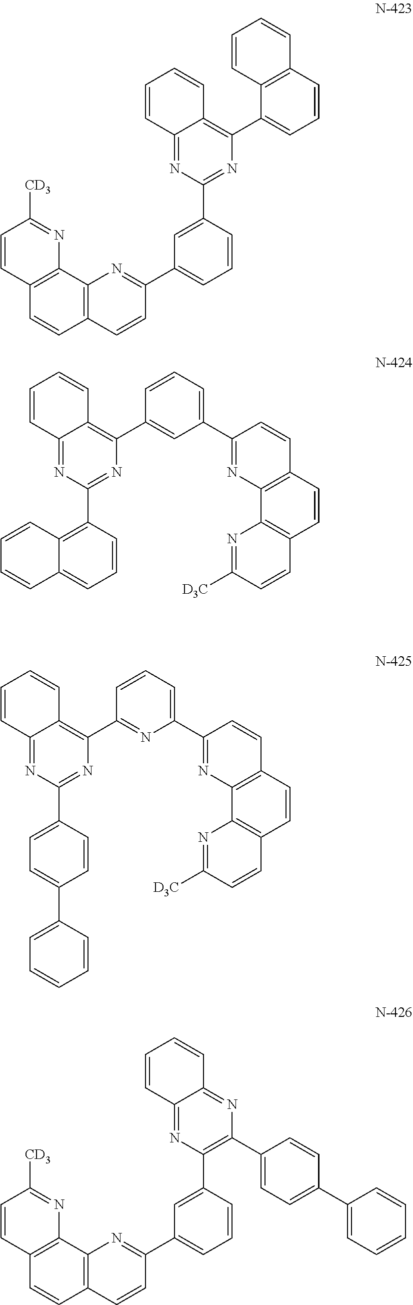 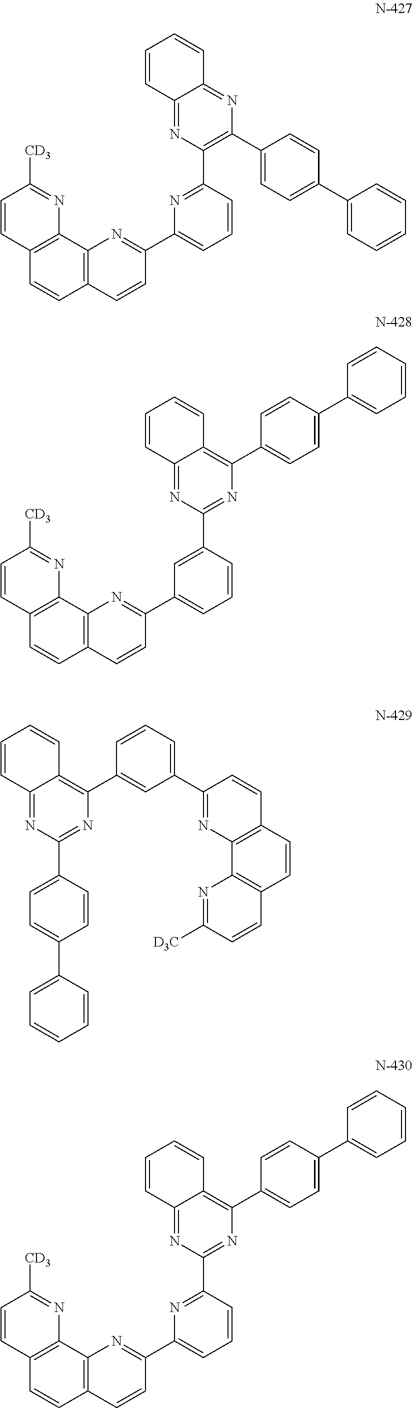 [SMILES: [2H]C([2H])([2H])c1ccc2ccc3ccc(-c4cccc(-c5nc6ccccc6nc5-c5ccc(-c6ccccc6)cc5)n4)nc3c2n1 ; [2H]C([2H])([2H])c1ccc2ccc3ccc(-c4cccc(-c5nc(-c6ccc(-c7ccccc7)cc6)c6ccccc6n5)c4)nc3c2n1 ; [2H]C([2H])([2H])c1ccc2ccc3ccc(-c4cccc(-c5nc(-c6ccc(-c7ccccc7)cc6)nc6ccccc56)c4)nc3c2n1 ; [2H]C([2H])([2H])c1ccc2ccc3ccc(-c4cccc(-c5nc(-c6ccc(-c7ccccc7)cc6)c6ccccc6n5)n4)nc3c2n1] 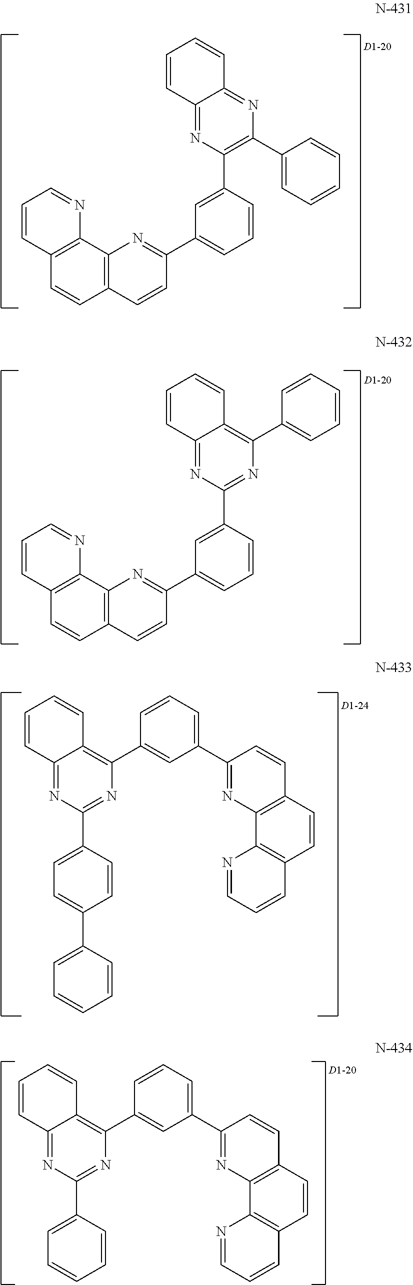 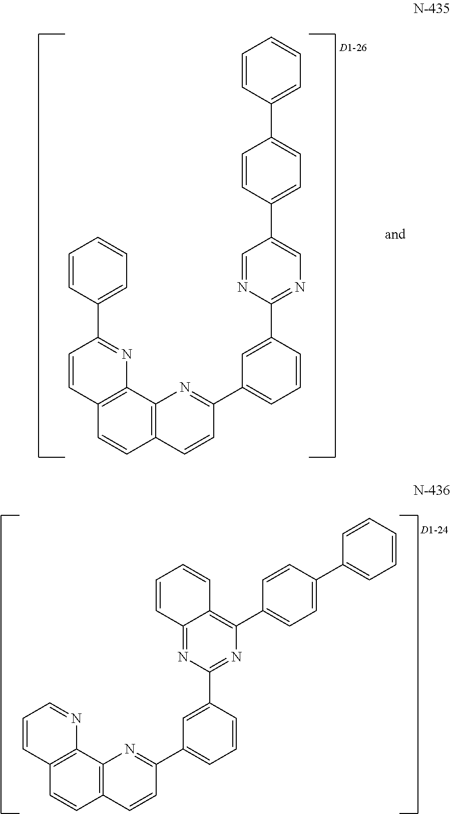
In the above compounds, Dn means that n hydrogens are replaced with deuterium, and n is from 1 to the maximum number of hydrogens in the compound.

12. An organic electroluminescent compound represented by the following Formula 2-1: 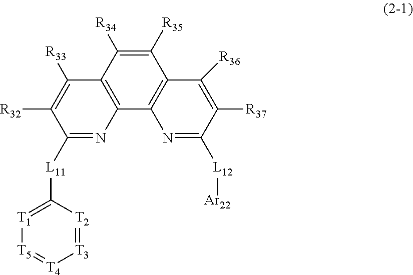
wherein,
R₃₂ to R₃₇ each independently represent, hydrogen, deuterium, a substituted or unsubstituted (C1-C30)alkyl, a substituted or unsubstituted (C3-C30)cycloalkyl, a substituted or unsubstituted (3- to 7-membered)heterocycloalkyl, or a substituted or unsubstituted (C6-C30)aryl; or may be linked to an adjacent substituent to form a ring;
L₁₁ represents a single bond, a substituted or unsubstituted (3- to 30-membered)heteroarylene, a substituted or unsubstituted naphthylene, a substituted or unsubstituted o-phenylene, a substituted or unsubstituted m-phenylene, or a substituted or unsubstituted p-phenylene;
L₁₂ represents a single bond, a substituted or unsubstituted (C1-C30)alkylene, a substituted or unsubstituted (C6-C30)arylene, a substituted or unsubstituted (3- to 30-membered)heteroarylene, or a substituted or unsubstituted (C3-C30)cycloalkylene;
T₁ to T₅ each independently represent, CRₐ or N; provided that at least one of T₁ to T₅ represent(s) N;
Rₐ represents hydrogen, deuterium, a substituted or unsubstituted (C1-C30)alkyl, a substituted or unsubstituted (C6-C30)aryl, or a substituted or unsubstituted (3- to 30-membered)heteroaryl; or may be linked to an adjacent substituent to form a ring;
Ar₂ represents hydrogen, deuterium, a substituted or unsubstituted (C1-C6)alkyl, a substituted or unsubstituted (C6-C30)aryl, or a substituted or unsubstituted (3- to 30-membered)heteroaryl;
provided that in Formula 2-1, the compounds where L₁₁ and L₁₂ are a single bond or phenylene, Ar₂₂ is naphthyl, dimethylfluorenyl, phenanthrenyl, pyrenyl, triphenylenyl, or fluoranthenyl, and 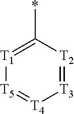
is pyridyl, pyrimidyl, quinolinyl, diphenyl-substituted triazinyl, dipyridyl-substituted pyridyl, or benzoquinolinyl, are excluded.

13. The organic electroluminescent compound of claim 12, 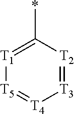
in Formula 2-1 is represented by the following Formula 2-1-1 or 2-1-2: 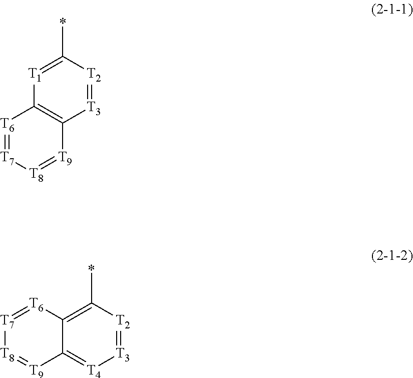
wherein,
T₁ to T₄ are as defined in claim 12;
T₆ to T₉ each independently represent, CRₐ; and
Rₐ is as defined in claim 12.

14. The organic electroluminescent compound of claim 12, wherein the organic electroluminescent compound represented by Formula 2-1 is selected from the following compounds:

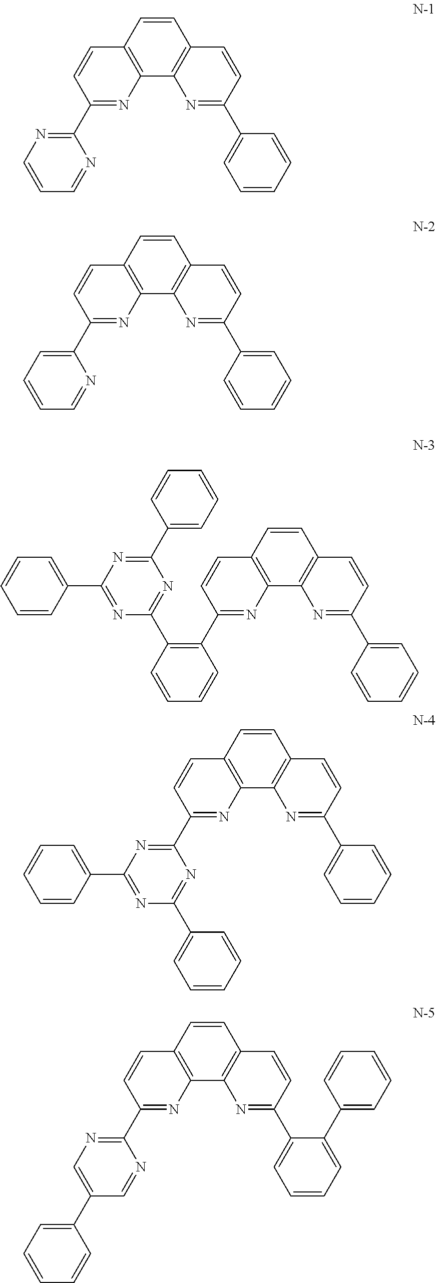

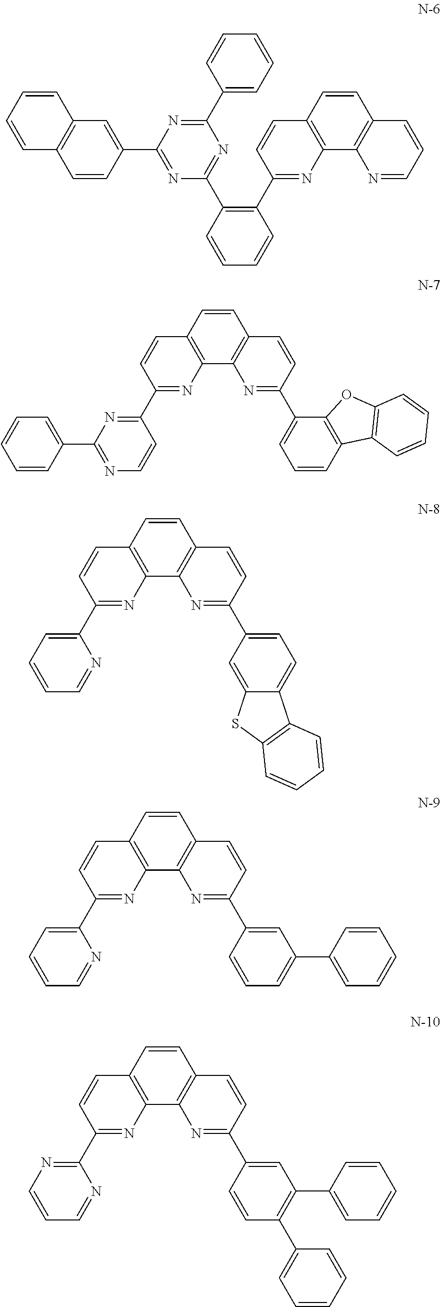

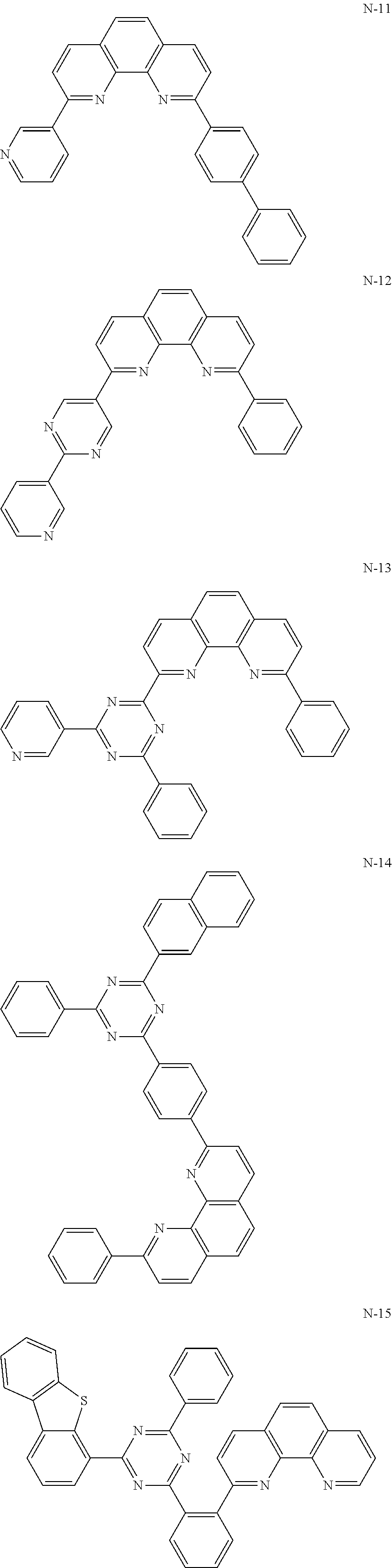

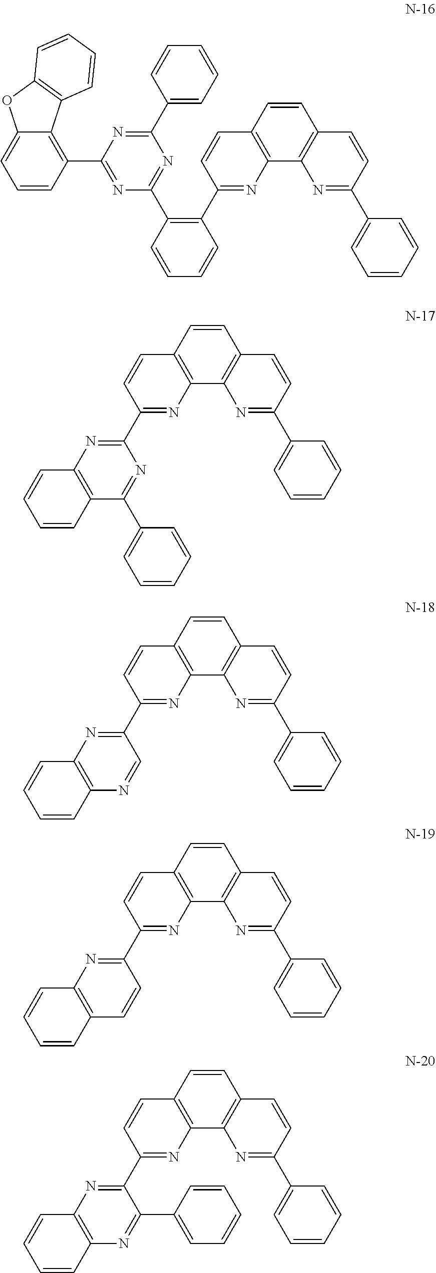

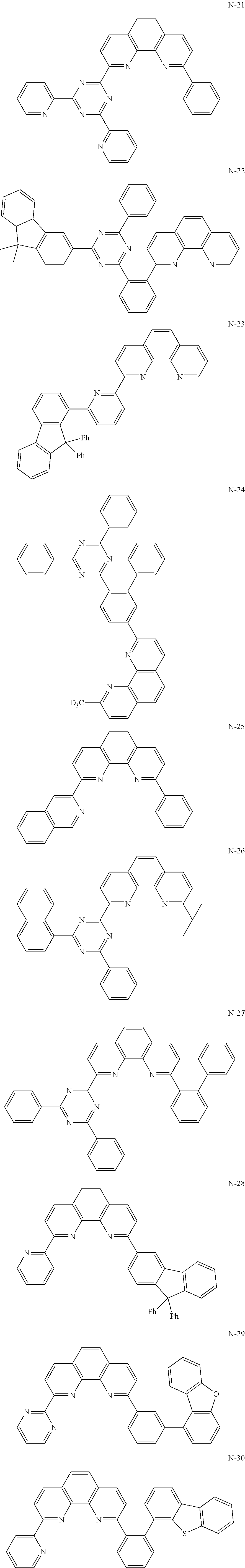

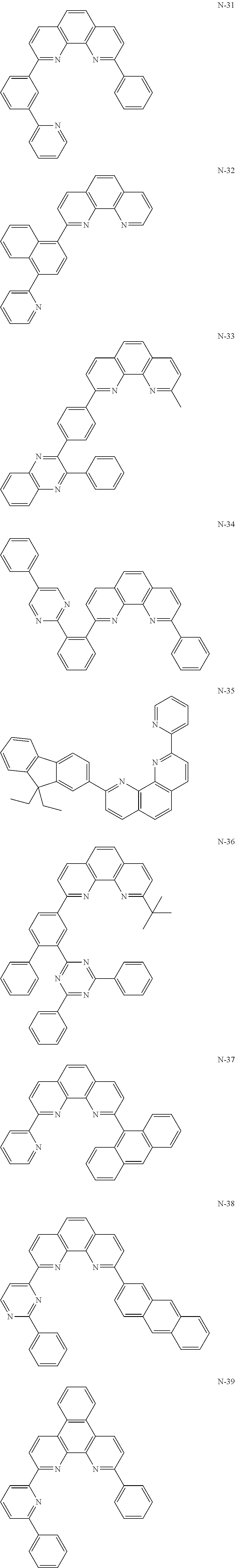

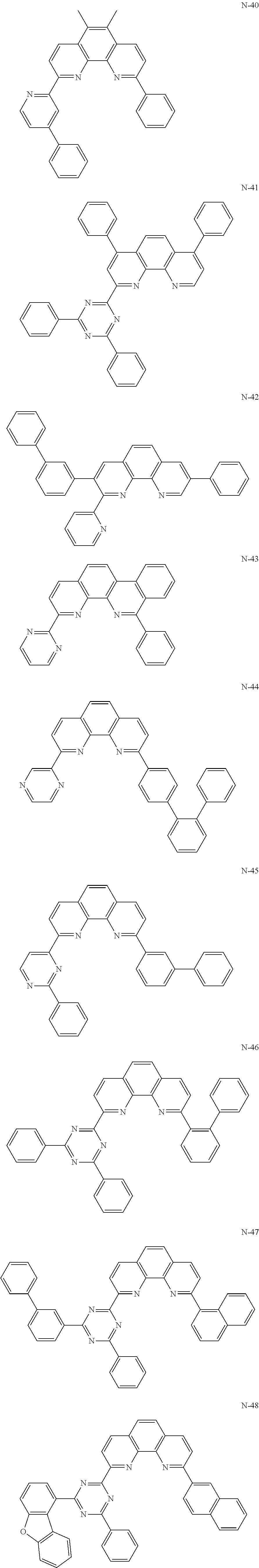

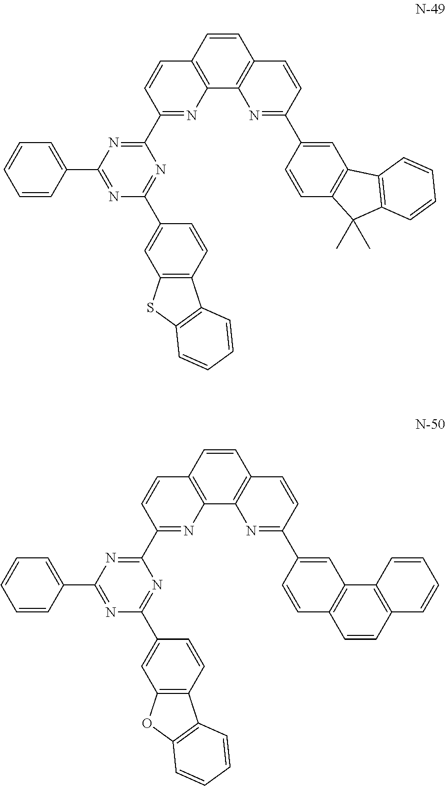 [SMILES: CC1(C)c2ccccc2-c2cc(-c3ccc4ccc5ccc(-c6nc(-c7ccccc7)nc(-c7ccc8c(c7)sc7ccccc78)n6)nc5c4n3)ccc21 ; c1ccc(-c2nc(-c3ccc4c(c3)oc3ccccc34)nc(-c3ccc4ccc5ccc(-c6ccc7ccc8ccccc8c7c6)nc5c4n3)n2)cc1]

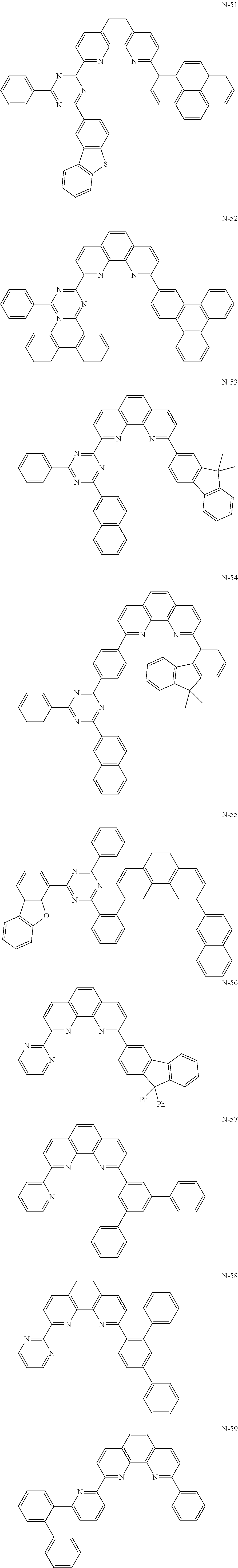

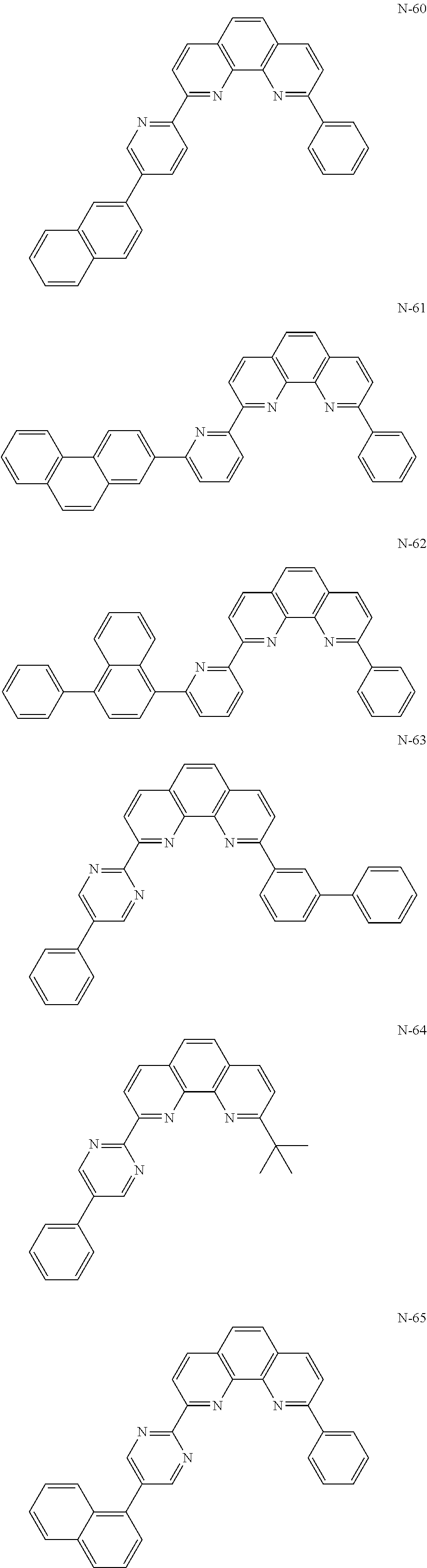 [SMILES: c1ccc(-c2ccc3ccc4ccc(-c5ccc(-c6ccc7ccccc7c6)cn5)nc4c3n2)cc1 ; c1ccc(-c2ccc3ccc4ccc(-c5cccc(-c6ccc7c(ccc8ccccc87)c6)n5)nc4c3n2)cc1 ; c1ccc(-c2ccc3ccc4ccc(-c5cccc(-c6ccc(-c7ccccc7)c7ccccc67)n5)nc4c3n2)cc1 ; c1ccc(-c2cnc(-c3ccc4ccc5ccc(-c6cccc(-c7ccccc7)c6)nc5c4n3)nc2)cc1 ; CC(C)(C)c1ccc2ccc3ccc(-c4ncc(-c5ccccc5)cn4)nc3c2n1 ; c1ccc(-c2ccc3ccc4ccc(-c5ncc(-c6cccc7ccccc67)cn5)nc4c3n2)cc1]

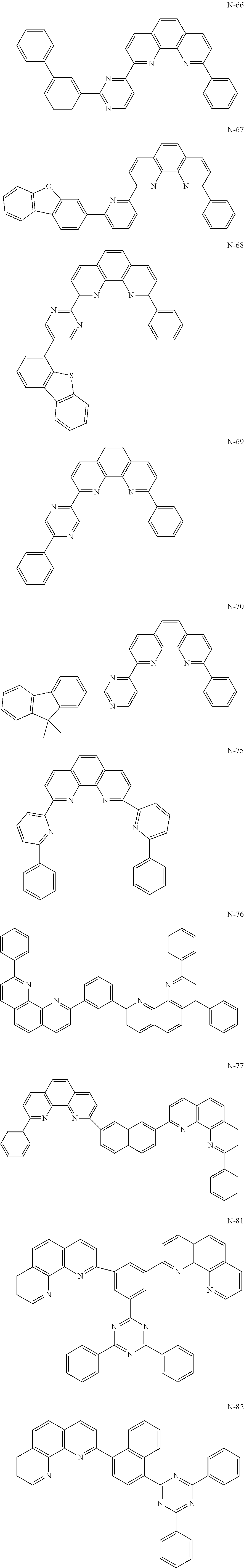

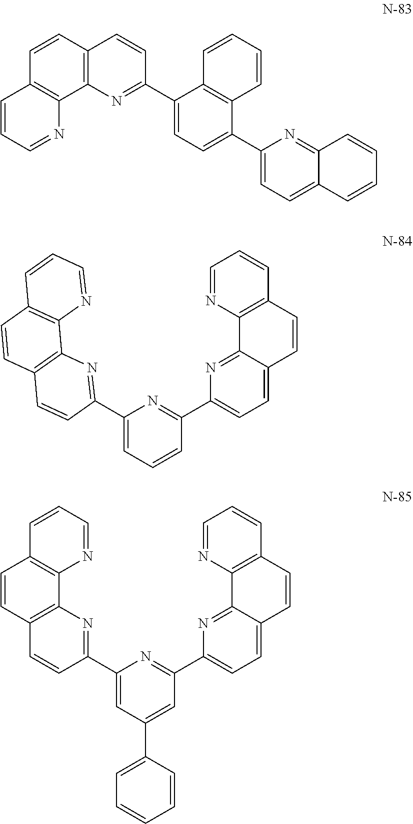 [SMILES: c1ccc2nc(-c3ccc(-c4ccc5ccc6cccnc6c5n4)c4ccccc34)ccc2c1 ; c1cc(-c2ccc3ccc4cccnc4c3n2)nc(-c2ccc3ccc4cccnc4c3n2)c1 ; c1ccc(-c2cc(-c3ccc4ccc5cccnc5c4n3)nc(-c3ccc4ccc5cccnc5c4n3)c2)cc1]

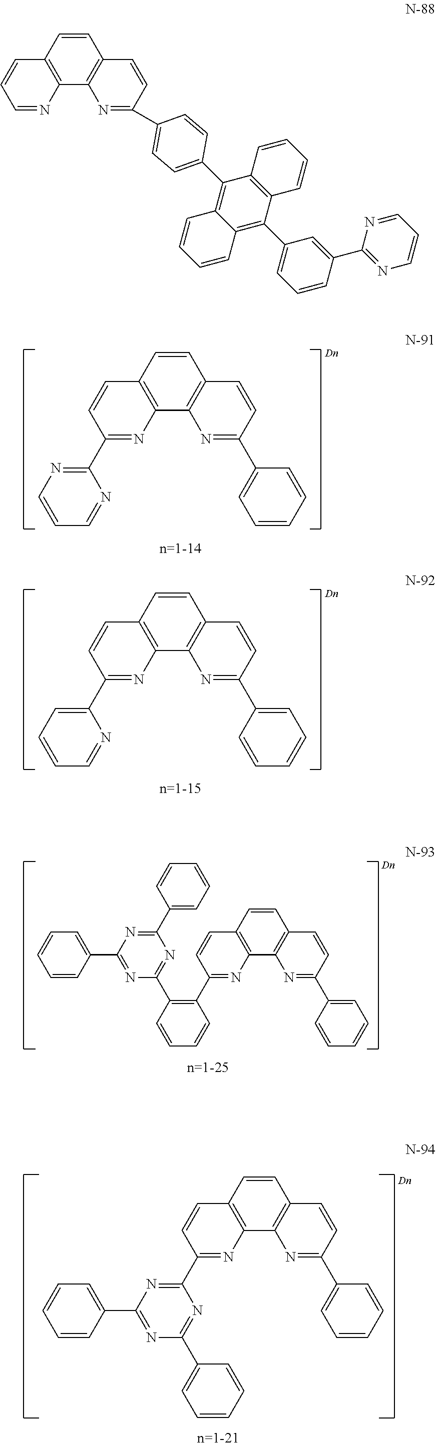

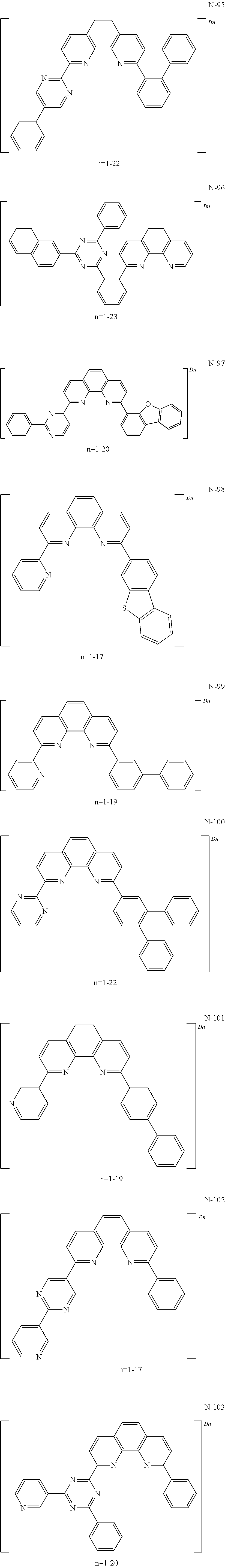

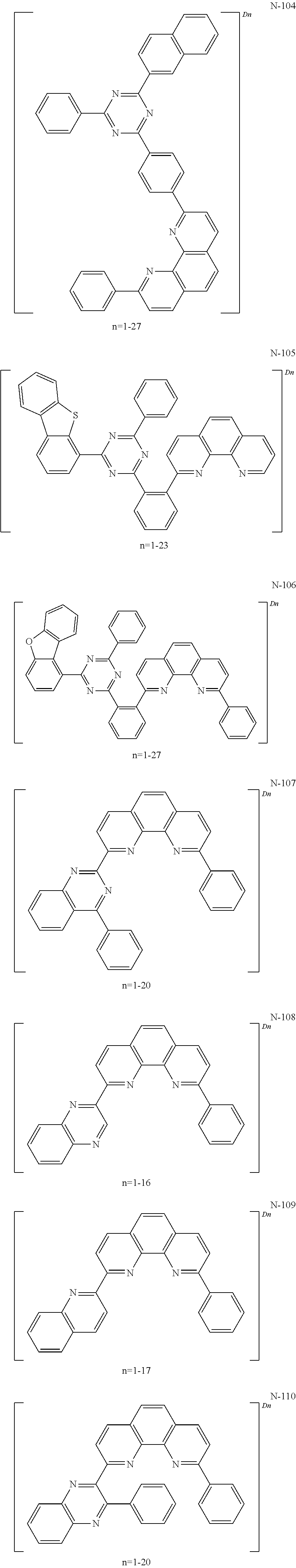

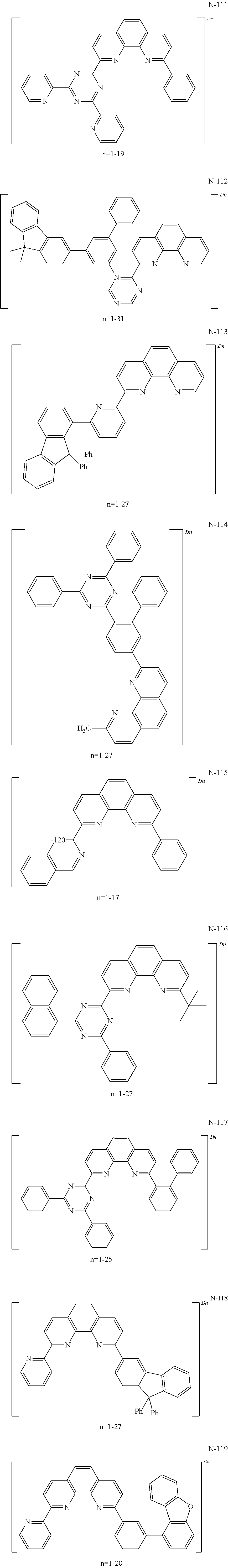

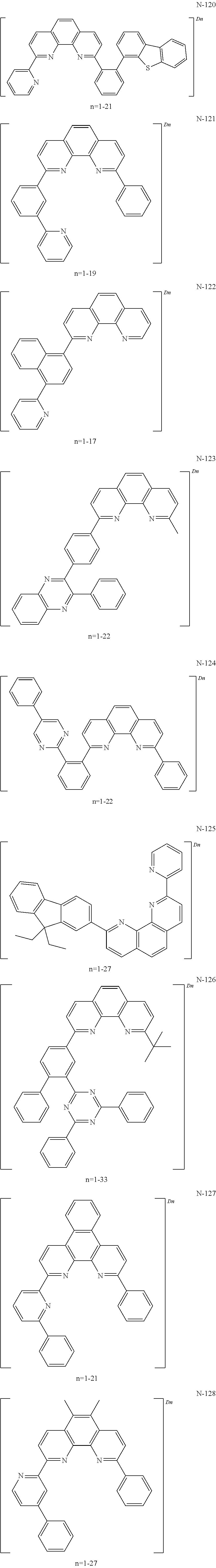

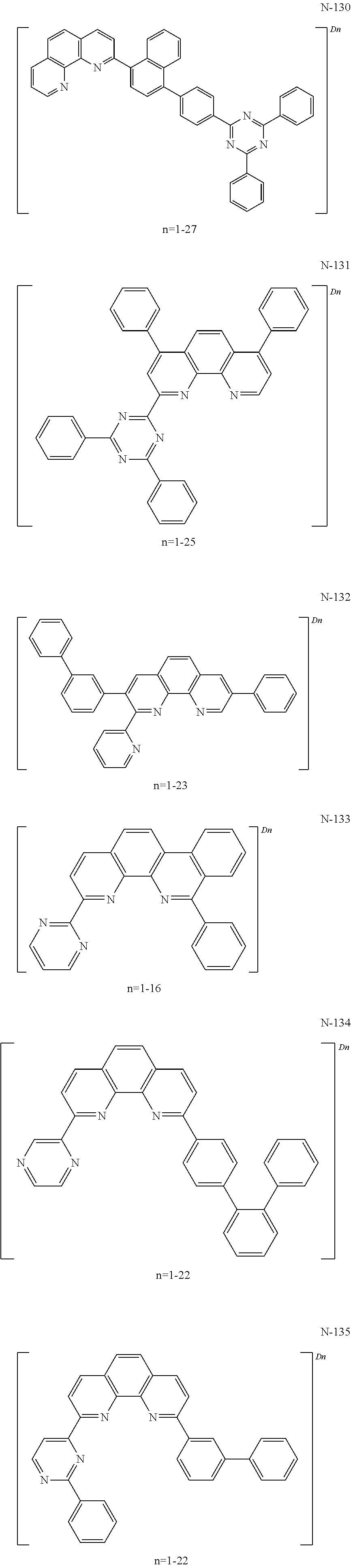

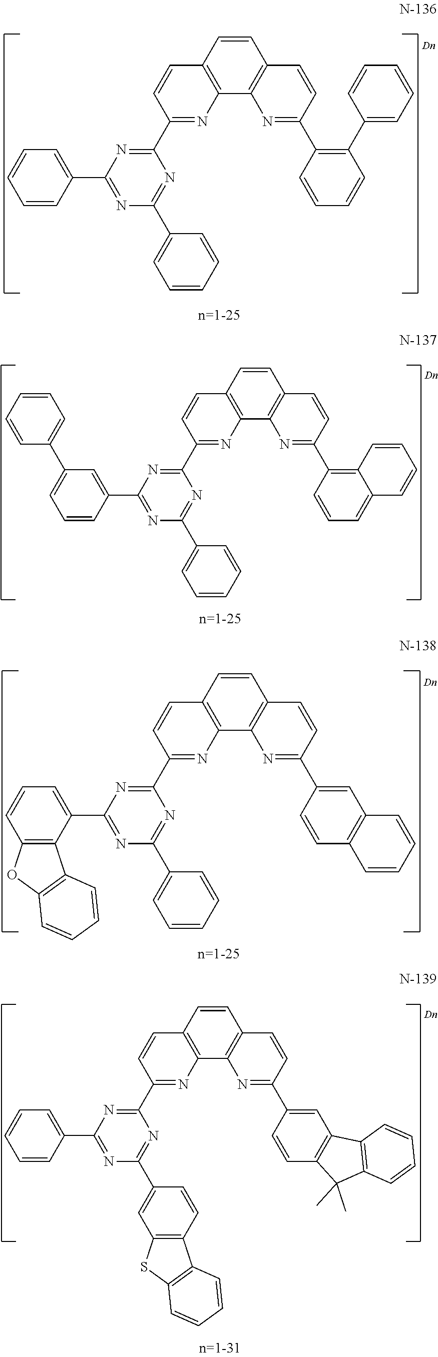

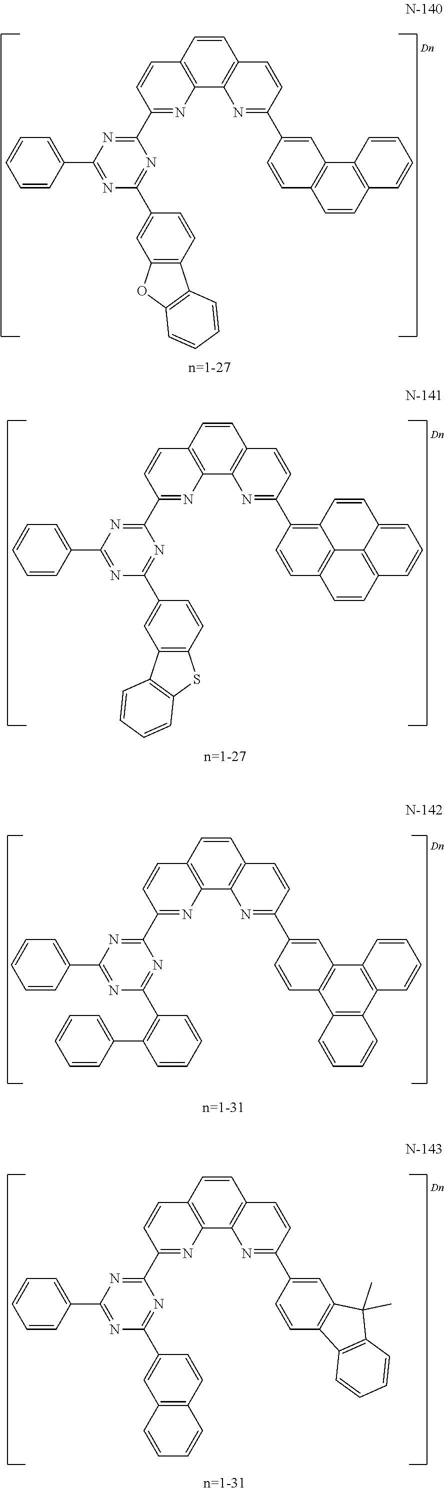

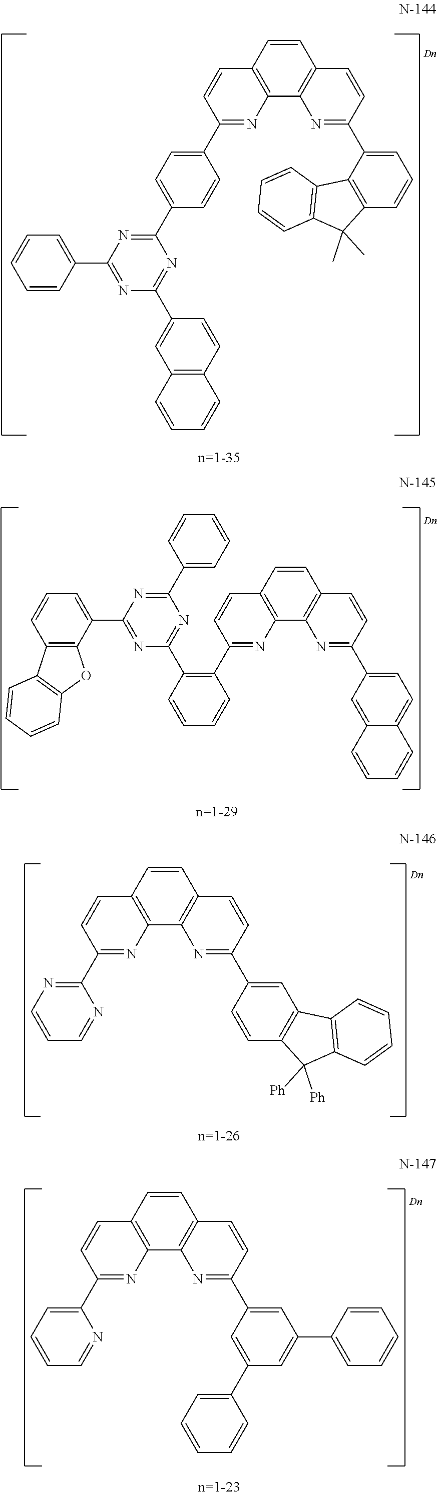

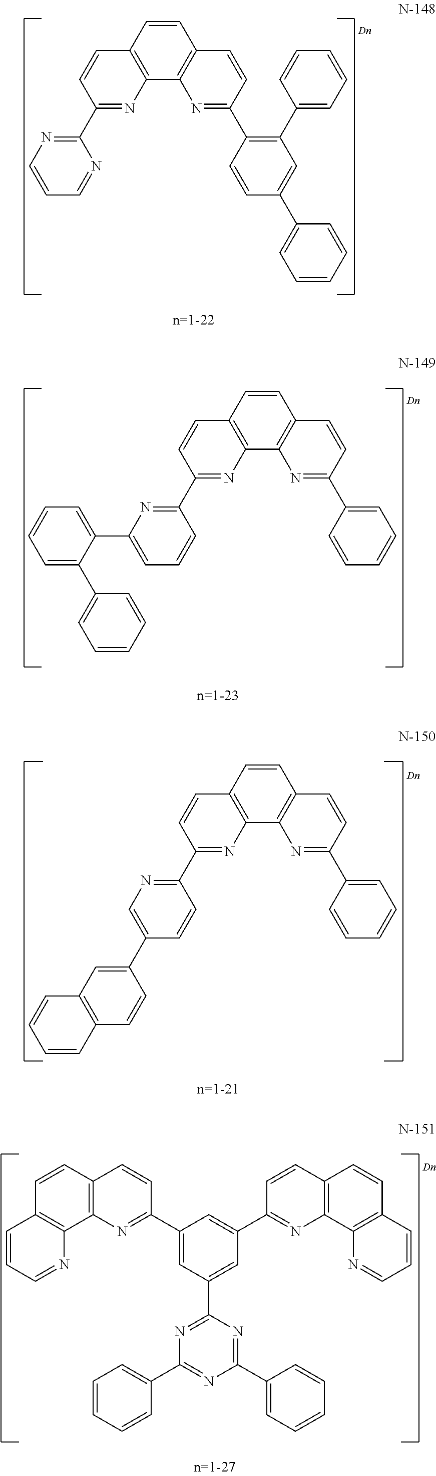

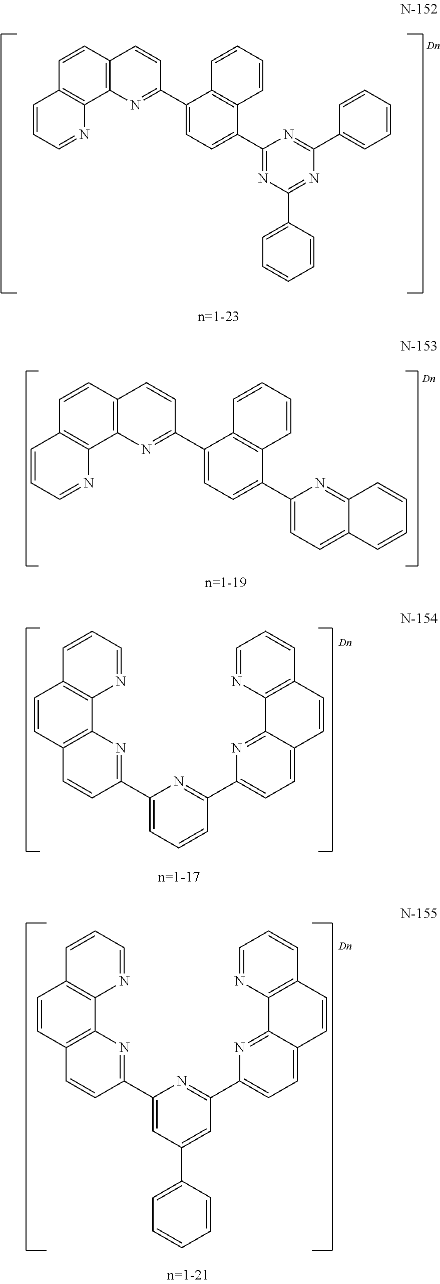

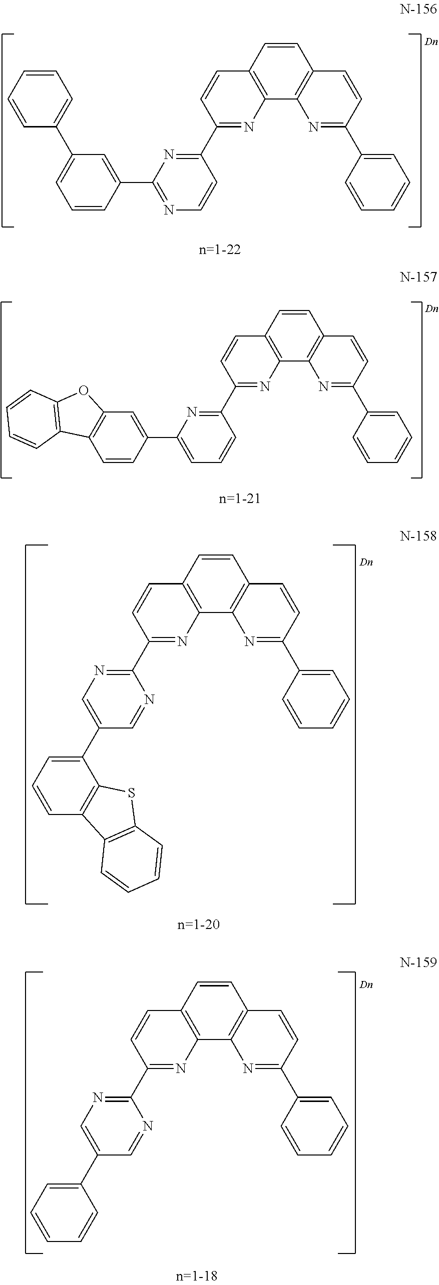

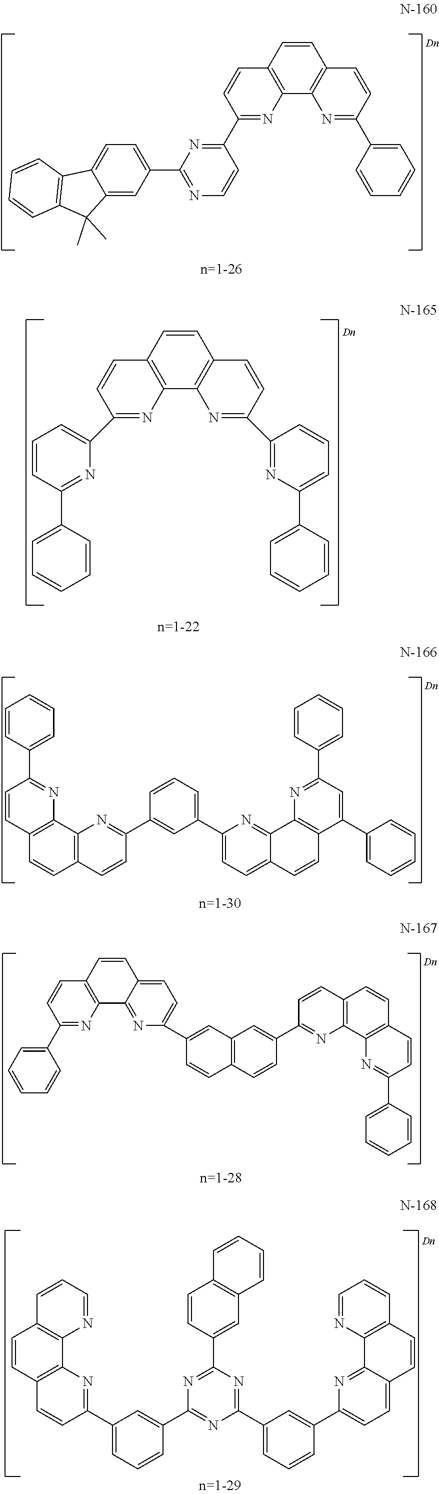

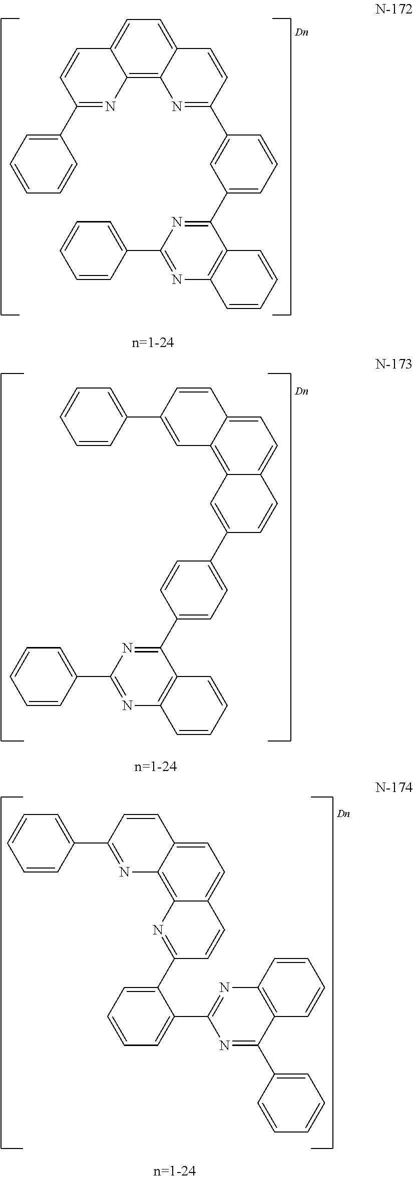

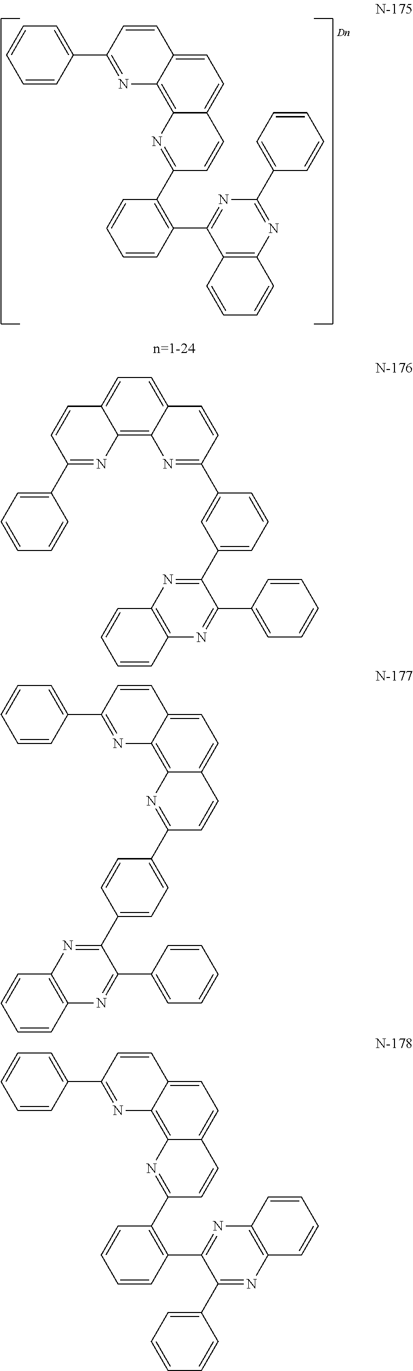

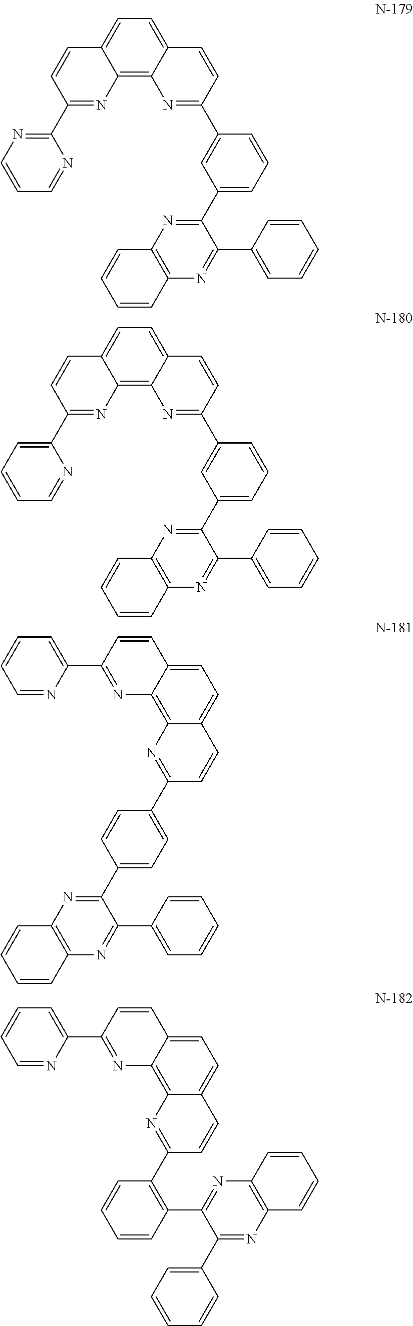

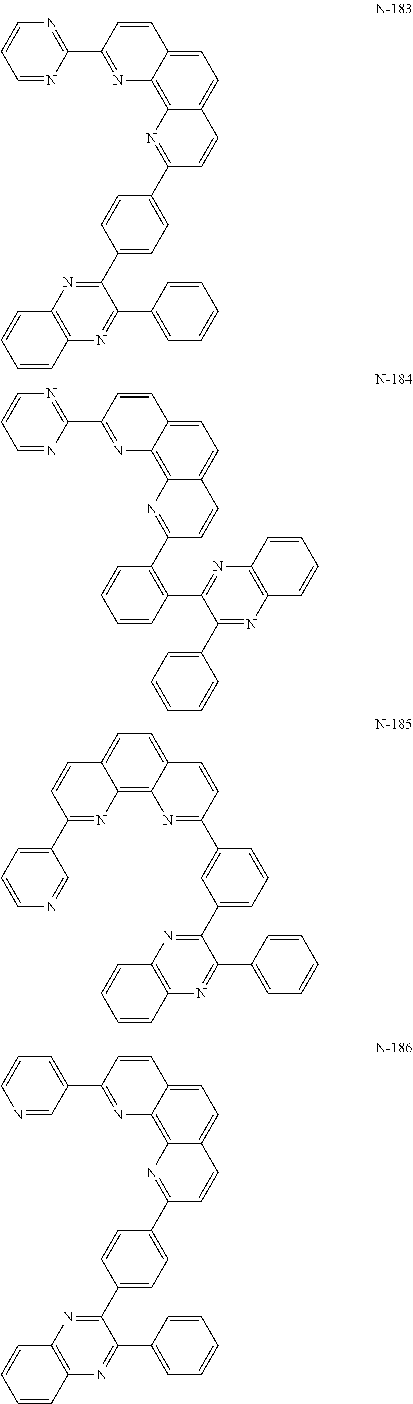

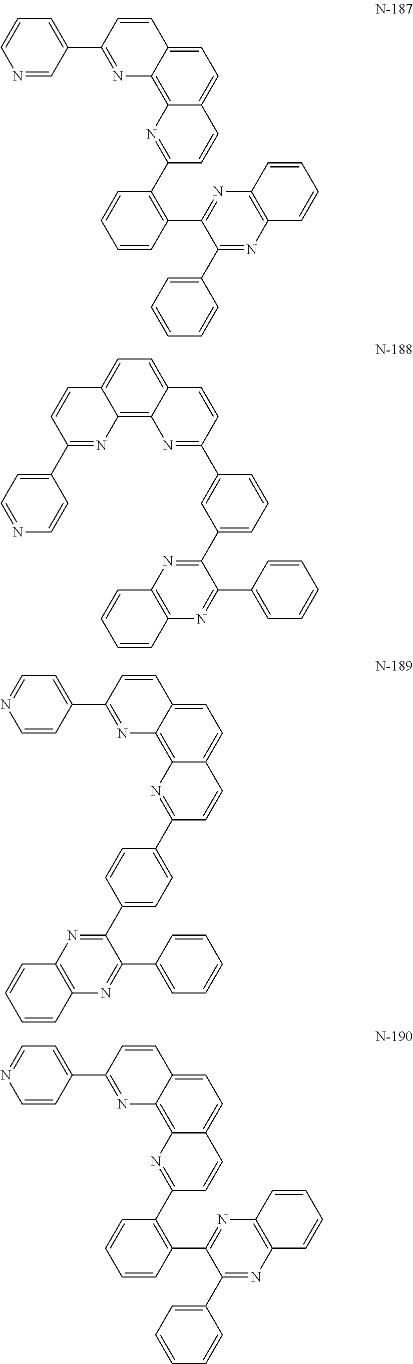

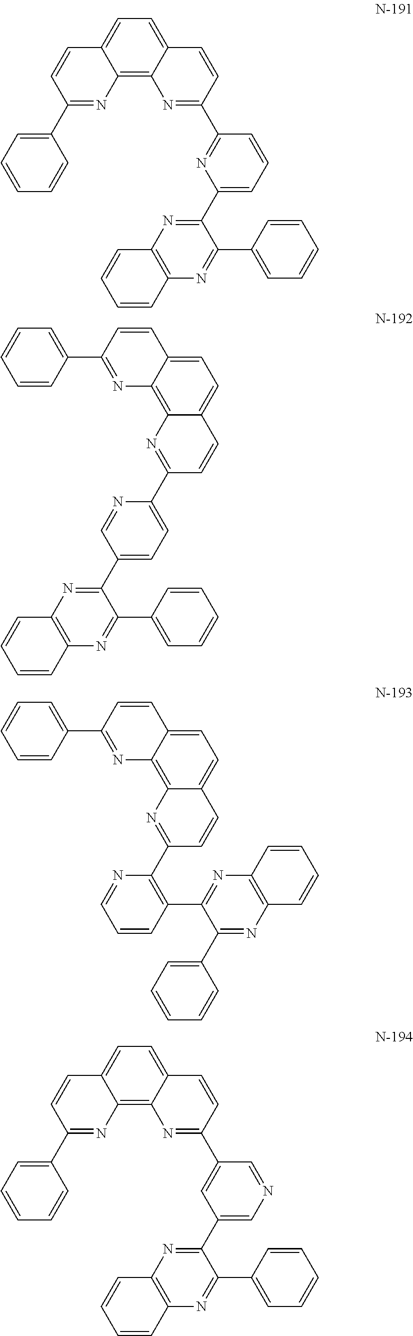

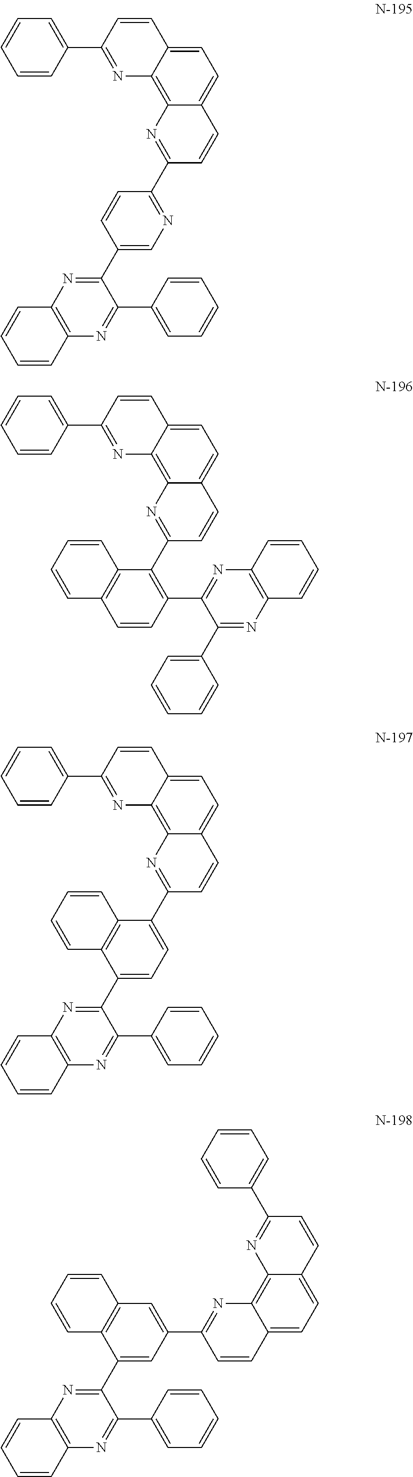

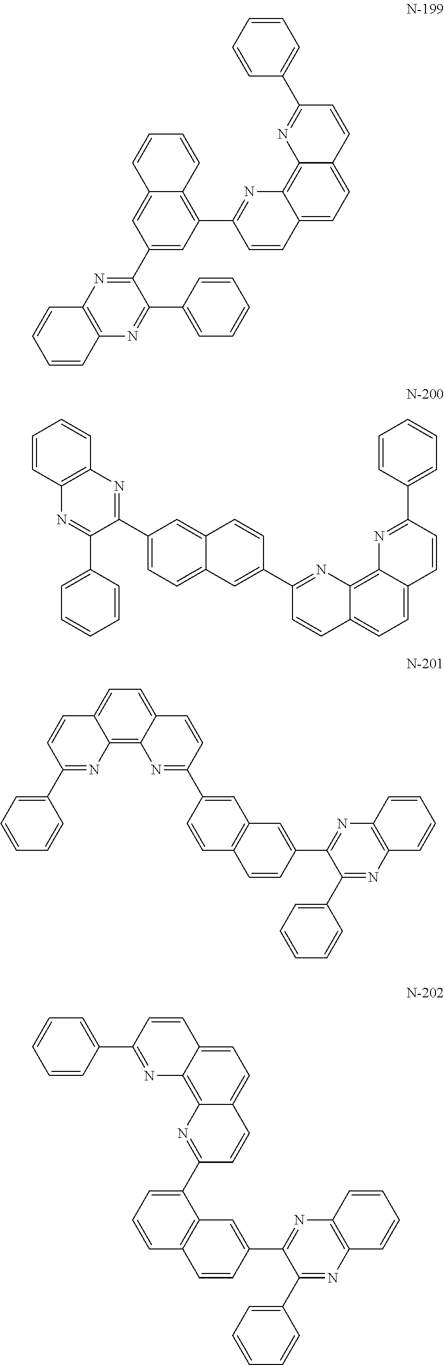 [SMILES: c1ccc(-c2ccc3ccc4ccc(-c5cc(-c6nc7ccccc7nc6-c6ccccc6)cc6ccccc56)nc4c3n2)cc1 ; c1ccc(-c2ccc3ccc4ccc(-c5ccc6cc(-c7nc8ccccc8nc7-c7ccccc7)ccc6c5)nc4c3n2)cc1 ; c1ccc(-c2ccc3ccc4ccc(-c5ccc6ccc(-c7nc8ccccc8nc7-c7ccccc7)cc6c5)nc4c3n2)cc1 ; c1ccc(-c2ccc3ccc4ccc(-c5cccc6ccc(-c7nc8ccccc8nc7-c7ccccc7)cc56)nc4c3n2)cc1]

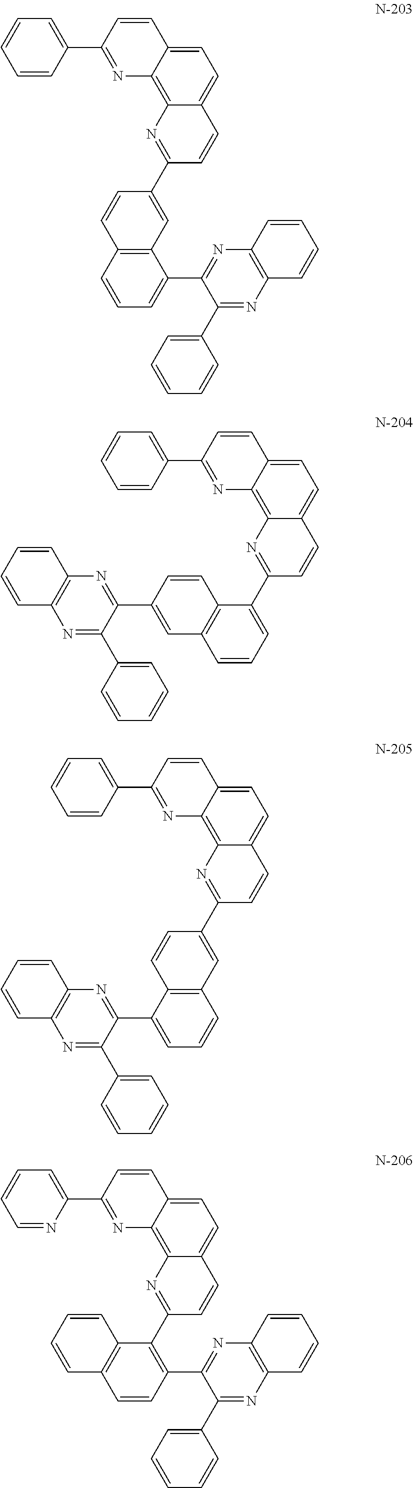

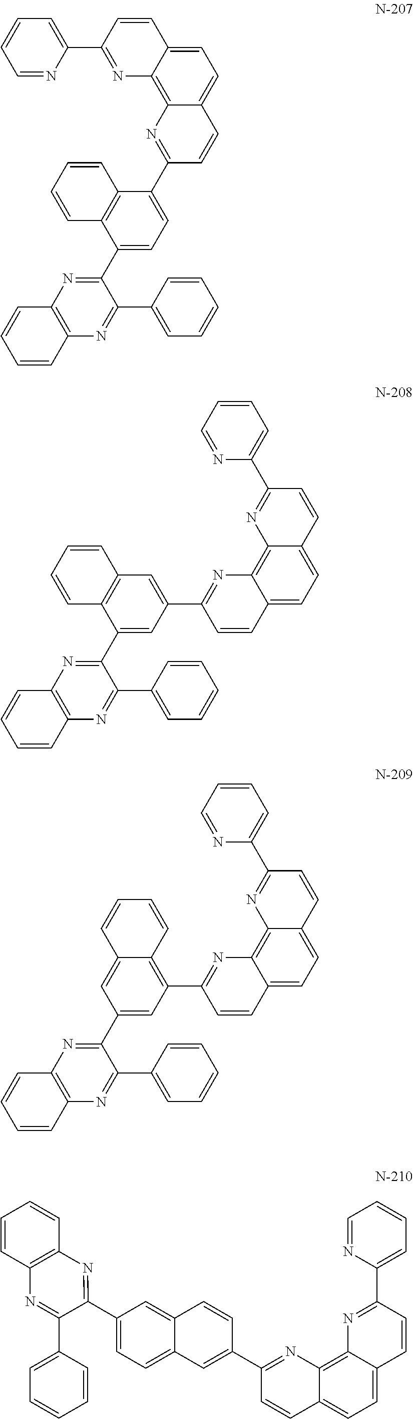 [SMILES: c1ccc(-c2nc3ccccc3nc2-c2ccc(-c3ccc4ccc5ccc(-c6ccccn6)nc5c4n3)c3ccccc23)cc1 ; c1ccc(-c2nc3ccccc3nc2-c2cc(-c3ccc4ccc5ccc(-c6ccccn6)nc5c4n3)cc3ccccc23)cc1 ; c1ccc(-c2nc3ccccc3nc2-c2cc(-c3ccc4ccc5ccc(-c6ccccn6)nc5c4n3)c3ccccc3c2)cc1 ; c1ccc(-c2nc3ccccc3nc2-c2ccc3cc(-c4ccc5ccc6ccc(-c7ccccn7)nc6c5n4)ccc3c2)cc1]

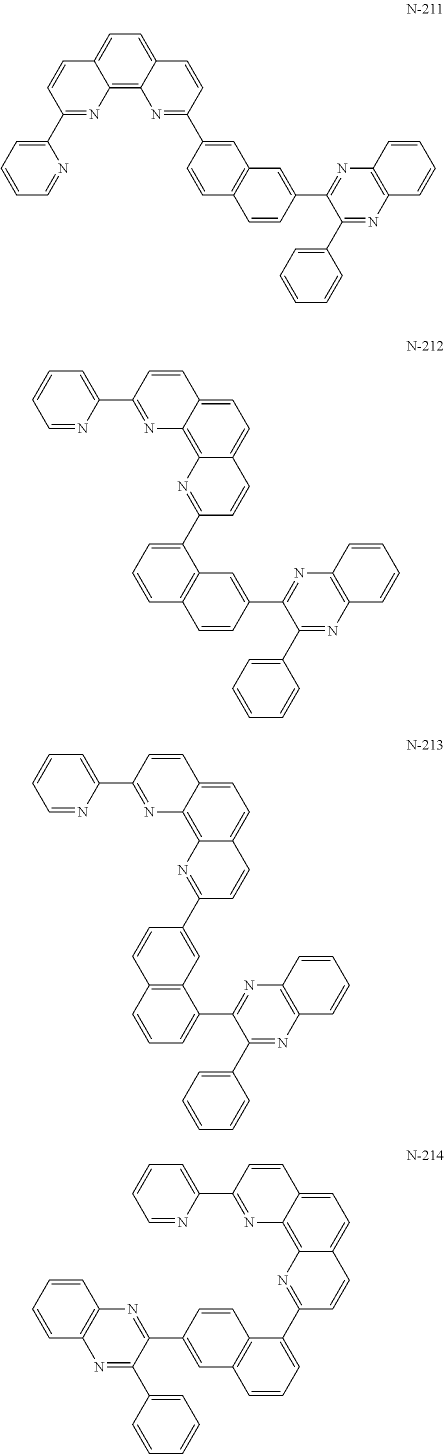

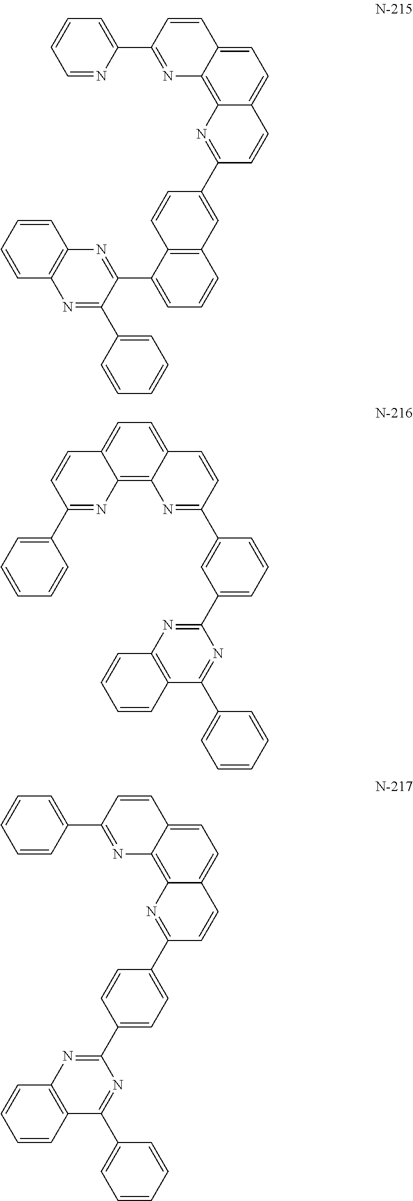

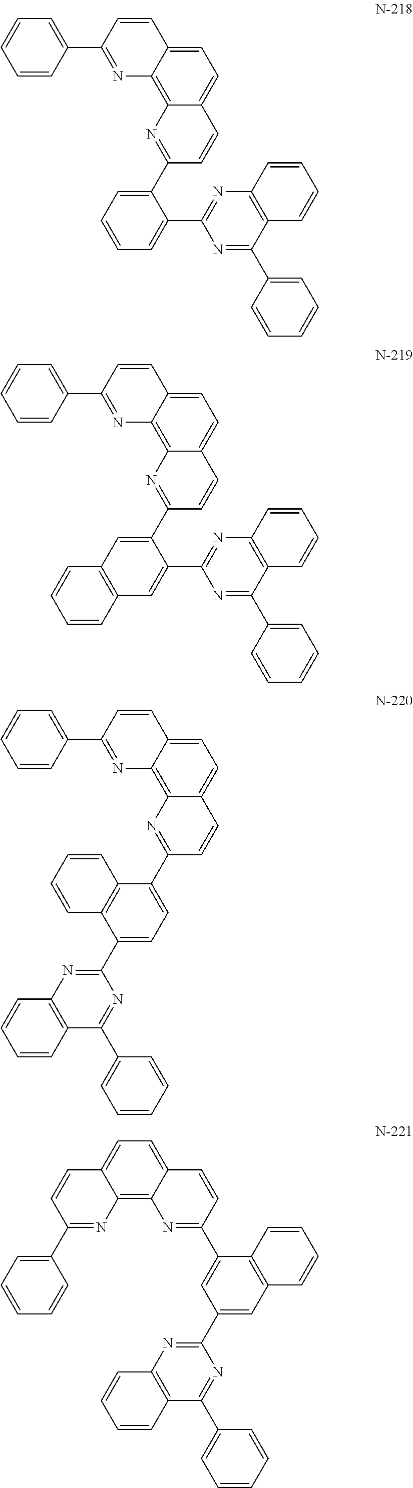

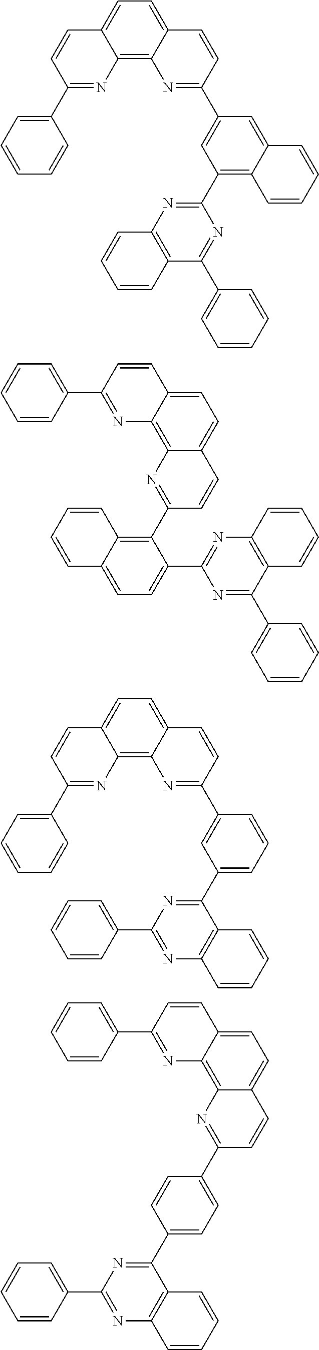

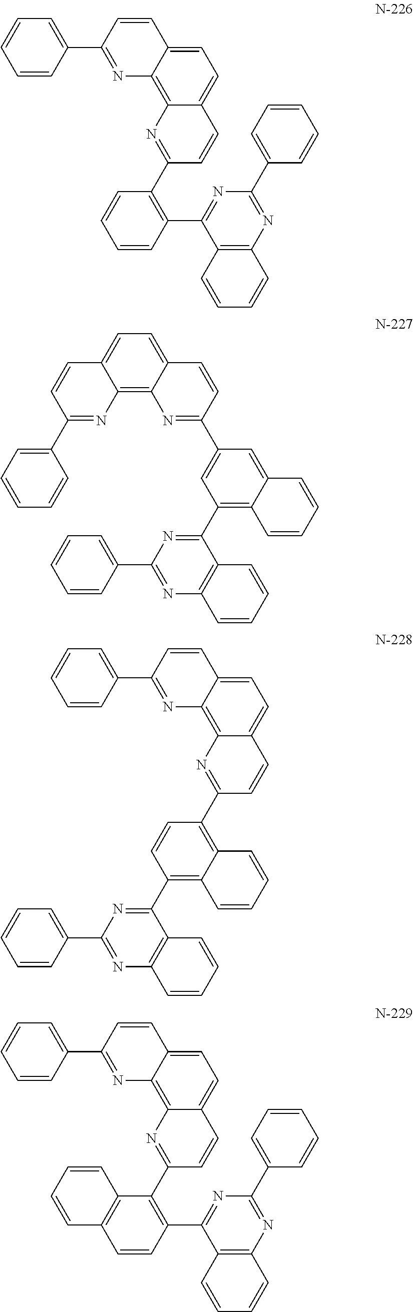

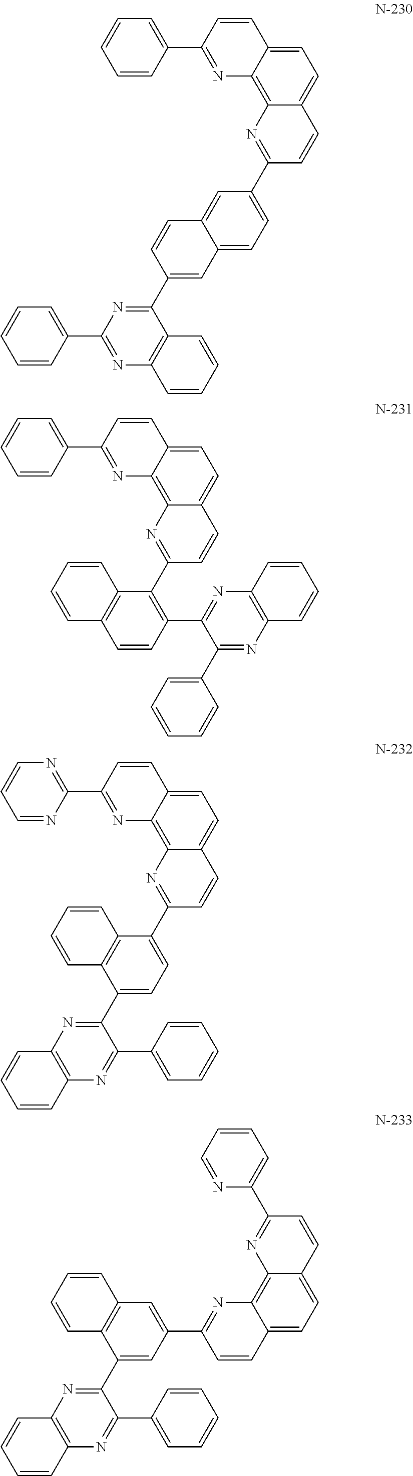

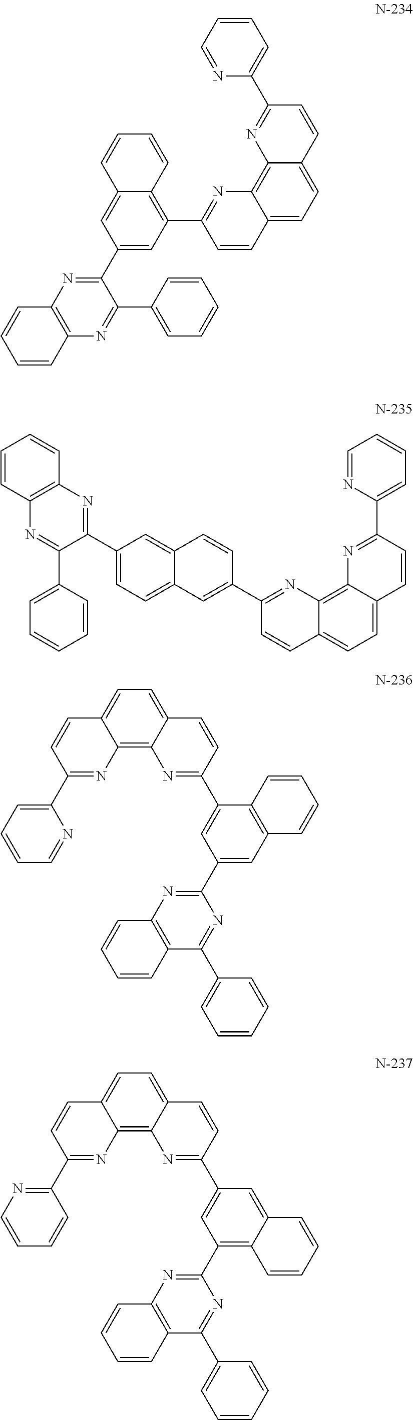 [SMILES: c1ccc(-c2nc3ccccc3nc2-c2cc(-c3ccc4ccc5ccc(-c6ccccn6)nc5c4n3)c3ccccc3c2)cc1 ; c1ccc(-c2nc3ccccc3nc2-c2ccc3cc(-c4ccc5ccc6ccc(-c7ccccn7)nc6c5n4)ccc3c2)cc1 ; c1ccc(-c2nc(-c3cc(-c4ccc5ccc6ccc(-c7ccccn7)nc6c5n4)c4ccccc4c3)nc3ccccc23)cc1 ; c1ccc(-c2nc(-c3cc(-c4ccc5ccc6ccc(-c7ccccn7)nc6c5n4)cc4ccccc34)nc3ccccc23)cc1]

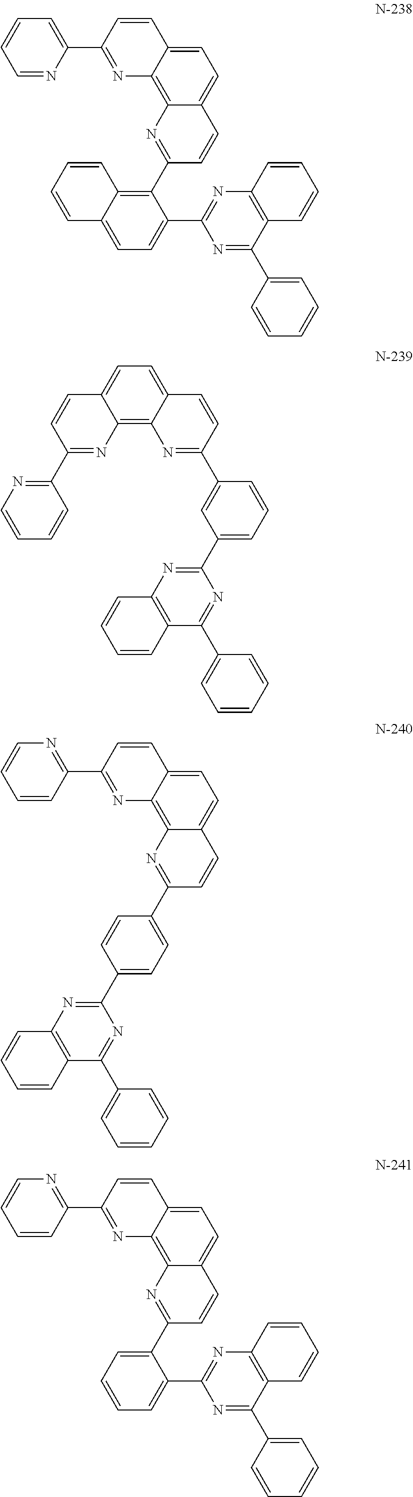

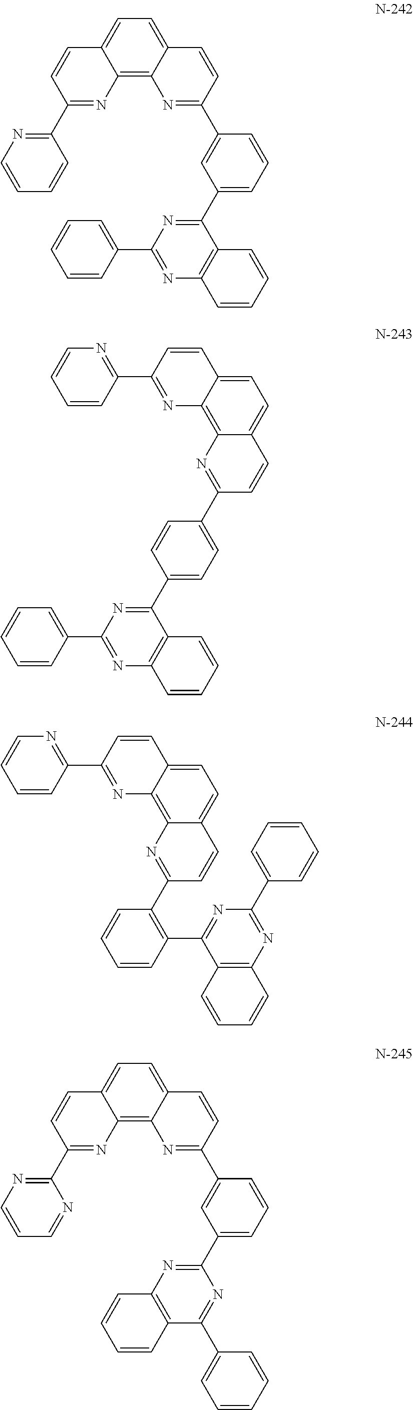

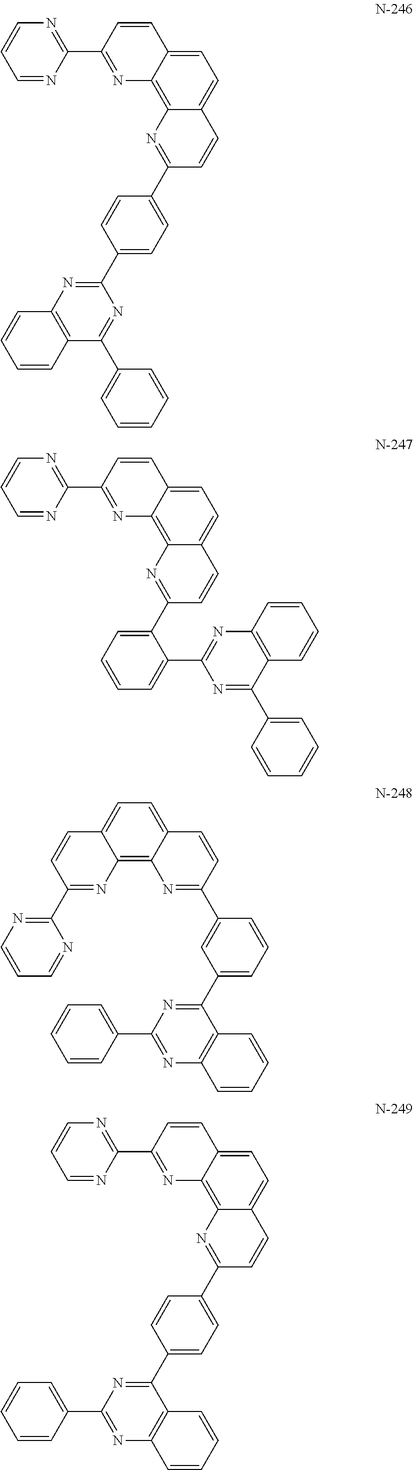

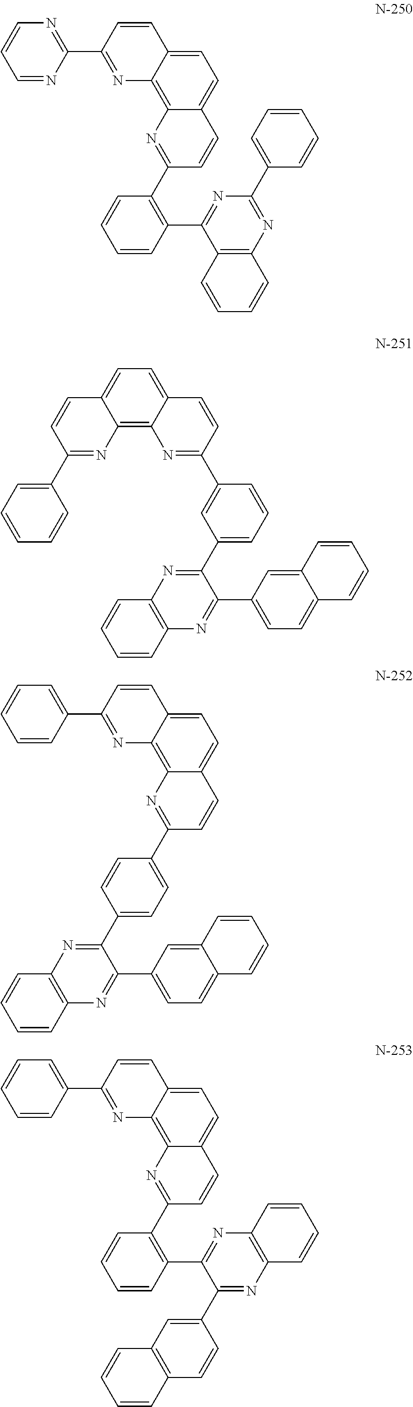

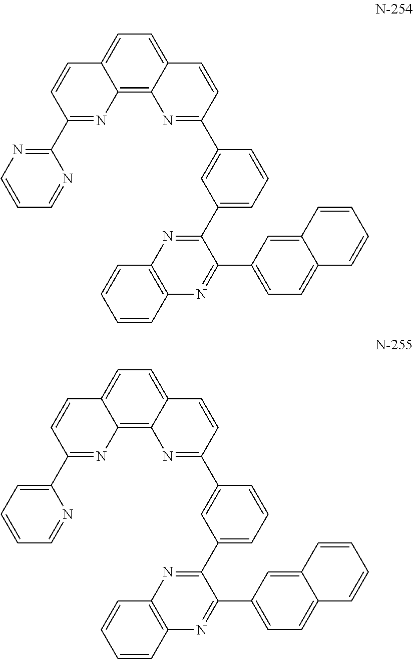 [SMILES: c1cnc(-c2ccc3ccc4ccc(-c5cccc(-c6nc7ccccc7nc6-c6ccc7ccccc7c6)c5)nc4c3n2)nc1 ; c1ccc(-c2ccc3ccc4ccc(-c5cccc(-c6nc7ccccc7nc6-c6ccc7ccccc7c6)c5)nc4c3n2)nc1]

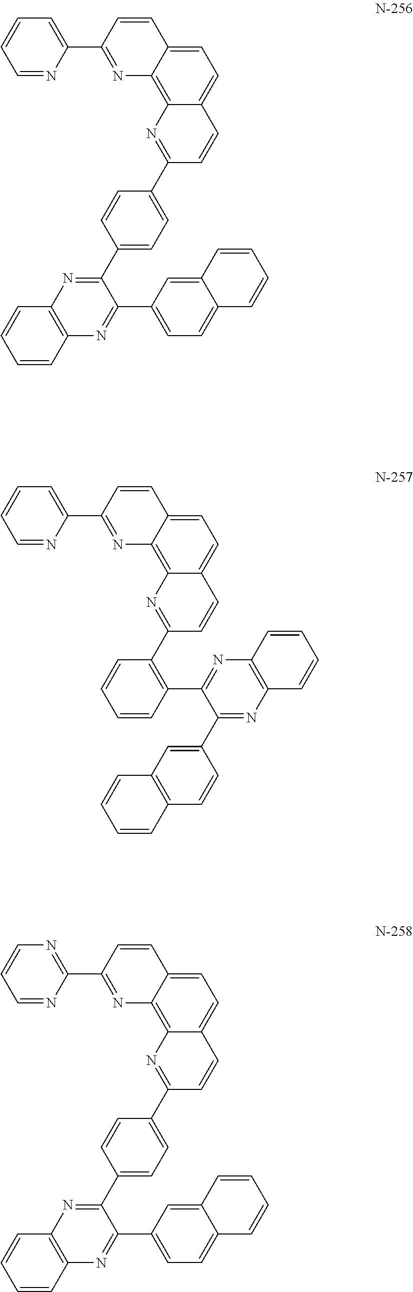 [SMILES: c1ccc(-c2ccc3ccc4ccc(-c5ccc(-c6nc7ccccc7nc6-c6ccc7ccccc7c6)cc5)nc4c3n2)nc1 ; c1ccc(-c2ccc3ccc4ccc(-c5ccccc5-c5nc6ccccc6nc5-c5ccc6ccccc6c5)nc4c3n2)nc1 ; c1cnc(-c2ccc3ccc4ccc(-c5ccc(-c6nc7ccccc7nc6-c6ccc7ccccc7c6)cc5)nc4c3n2)nc1]

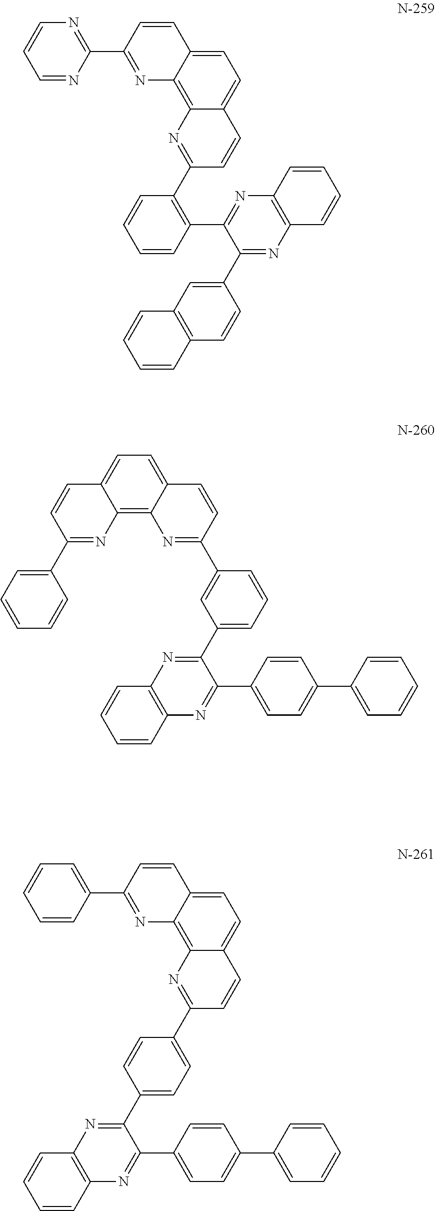 [SMILES: c1cnc(-c2ccc3ccc4ccc(-c5ccccc5-c5nc6ccccc6nc5-c5ccc6ccccc6c5)nc4c3n2)nc1 ; c1ccc(-c2ccc(-c3nc4ccccc4nc3-c3cccc(-c4ccc5ccc6ccc(-c7ccccc7)nc6c5n4)c3)cc2)cc1 ; c1ccc(-c2ccc(-c3nc4ccccc4nc3-c3ccc(-c4ccc5ccc6ccc(-c7ccccc7)nc6c5n4)cc3)cc2)cc1]

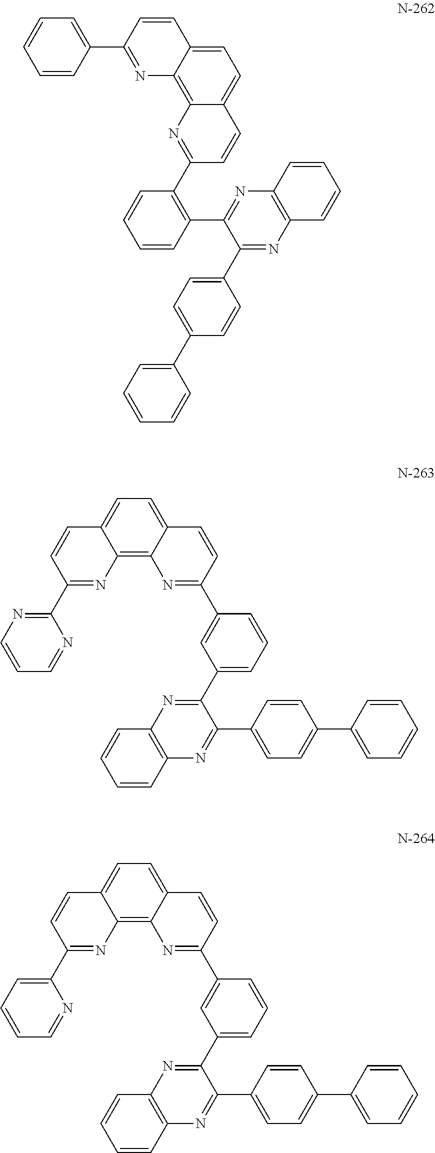 [SMILES: c1ccc(-c2ccc(-c3nc4ccccc4nc3-c3ccccc3-c3ccc4ccc5ccc(-c6ccccc6)nc5c4n3)cc2)cc1 ; c1ccc(-c2ccc(-c3nc4ccccc4nc3-c3cccc(-c4ccc5ccc6ccc(-c7ncccn7)nc6c5n4)c3)cc2)cc1 ; c1ccc(-c2ccc(-c3nc4ccccc4nc3-c3cccc(-c4ccc5ccc6ccc(-c7ccccn7)nc6c5n4)c3)cc2)cc1]

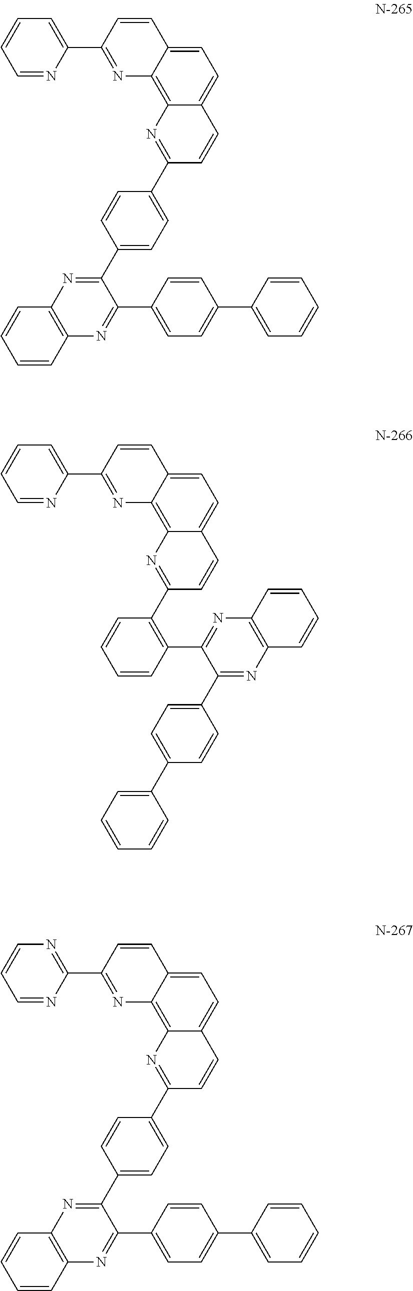 [SMILES: c1ccc(-c2ccc(-c3nc4ccccc4nc3-c3ccc(-c4ccc5ccc6ccc(-c7ccccn7)nc6c5n4)cc3)cc2)cc1 ; c1ccc(-c2ccc(-c3nc4ccccc4nc3-c3ccccc3-c3ccc4ccc5ccc(-c6ccccn6)nc5c4n3)cc2)cc1 ; c1ccc(-c2ccc(-c3nc4ccccc4nc3-c3ccc(-c4ccc5ccc6ccc(-c7ncccn7)nc6c5n4)cc3)cc2)cc1]

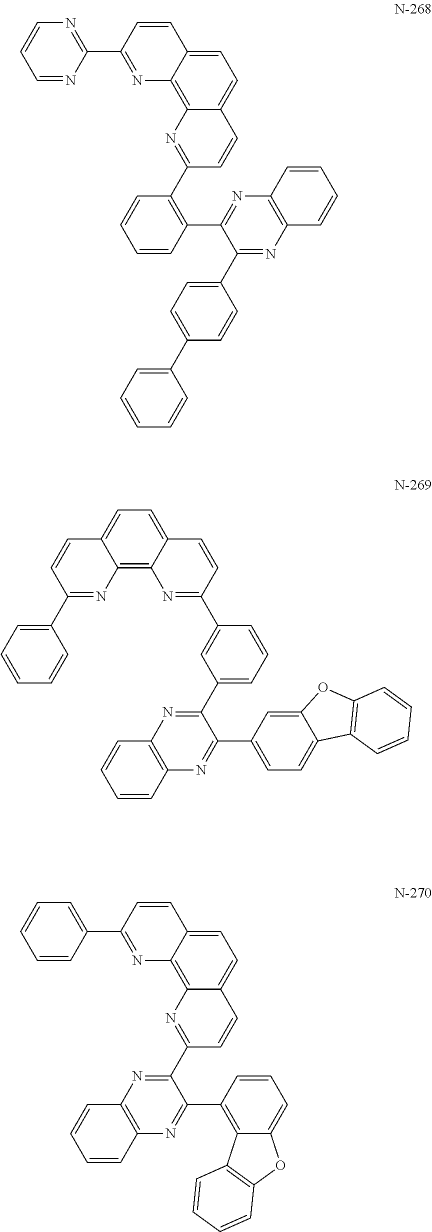 [SMILES: c1ccc(-c2ccc(-c3nc4ccccc4nc3-c3ccccc3-c3ccc4ccc5ccc(-c6ncccn6)nc5c4n3)cc2)cc1 ; c1ccc(-c2ccc3ccc4ccc(-c5cccc(-c6nc7ccccc7nc6-c6ccc7c(c6)oc6ccccc67)c5)nc4c3n2)cc1 ; c1ccc(-c2ccc3ccc4ccc(-c5nc6ccccc6nc5-c5cccc6oc7ccccc7c56)nc4c3n2)cc1]

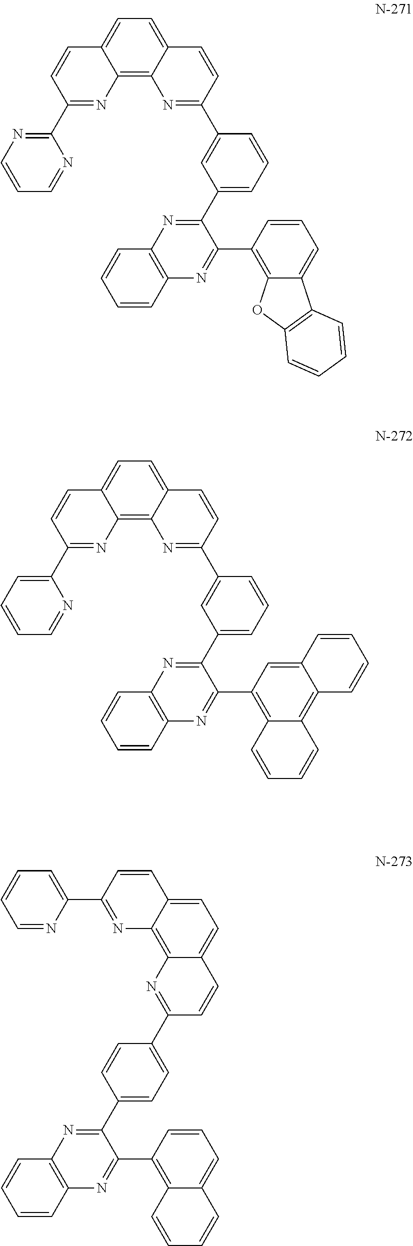 [SMILES: c1cnc(-c2ccc3ccc4ccc(-c5cccc(-c6nc7ccccc7nc6-c6cccc7c6oc6ccccc67)c5)nc4c3n2)nc1 ; c1ccc(-c2ccc3ccc4ccc(-c5cccc(-c6nc7ccccc7nc6-c6cc7ccccc7c7ccccc67)c5)nc4c3n2)nc1 ; c1ccc(-c2ccc3ccc4ccc(-c5ccc(-c6nc7ccccc7nc6-c6cccc7ccccc67)cc5)nc4c3n2)nc1]

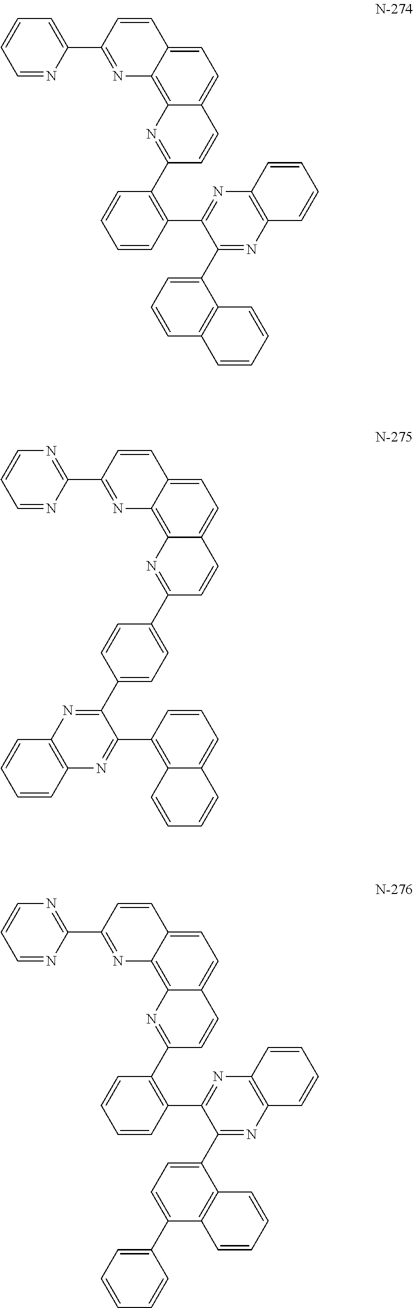 [SMILES: c1ccc(-c2ccc3ccc4ccc(-c5ccccc5-c5nc6ccccc6nc5-c5cccc6ccccc56)nc4c3n2)nc1 ; c1cnc(-c2ccc3ccc4ccc(-c5ccc(-c6nc7ccccc7nc6-c6cccc7ccccc67)cc5)nc4c3n2)nc1 ; c1ccc(-c2ccc(-c3nc4ccccc4nc3-c3ccccc3-c3ccc4ccc5ccc(-c6ncccn6)nc5c4n3)c3ccccc23)cc1]

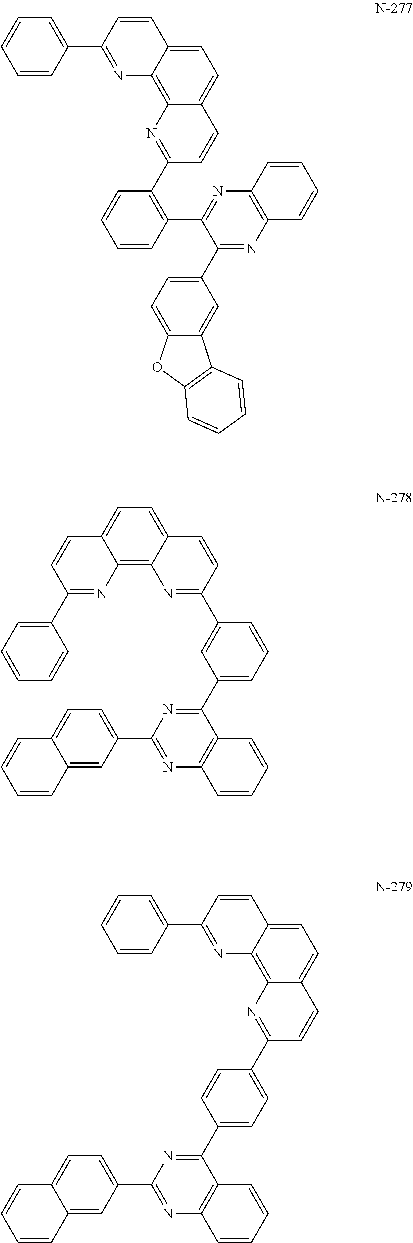 [SMILES: c1ccc(-c2ccc3ccc4ccc(-c5ccccc5-c5nc6ccccc6nc5-c5ccc6oc7ccccc7c6c5)nc4c3n2)cc1 ; c1ccc(-c2ccc3ccc4ccc(-c5cccc(-c6nc(-c7ccc8ccccc8c7)nc7ccccc67)c5)nc4c3n2)cc1 ; c1ccc(-c2ccc3ccc4ccc(-c5ccc(-c6nc(-c7ccc8ccccc8c7)nc7ccccc67)cc5)nc4c3n2)cc1]

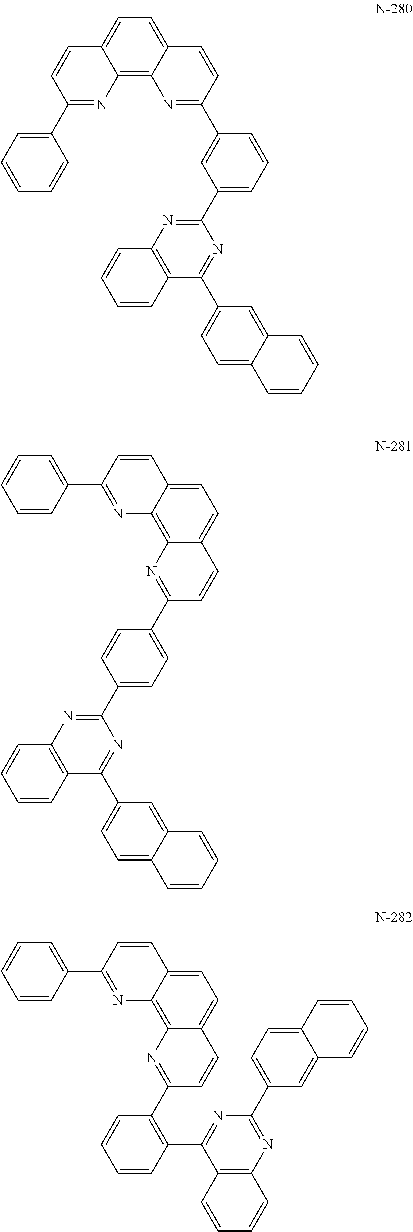 [SMILES: c1ccc(-c2ccc3ccc4ccc(-c5cccc(-c6nc(-c7ccc8ccccc8c7)c7ccccc7n6)c5)nc4c3n2)cc1 ; c1ccc(-c2ccc3ccc4ccc(-c5ccc(-c6nc(-c7ccc8ccccc8c7)c7ccccc7n6)cc5)nc4c3n2)cc1 ; c1ccc(-c2ccc3ccc4ccc(-c5ccccc5-c5nc(-c6ccc7ccccc7c6)nc6ccccc56)nc4c3n2)cc1]

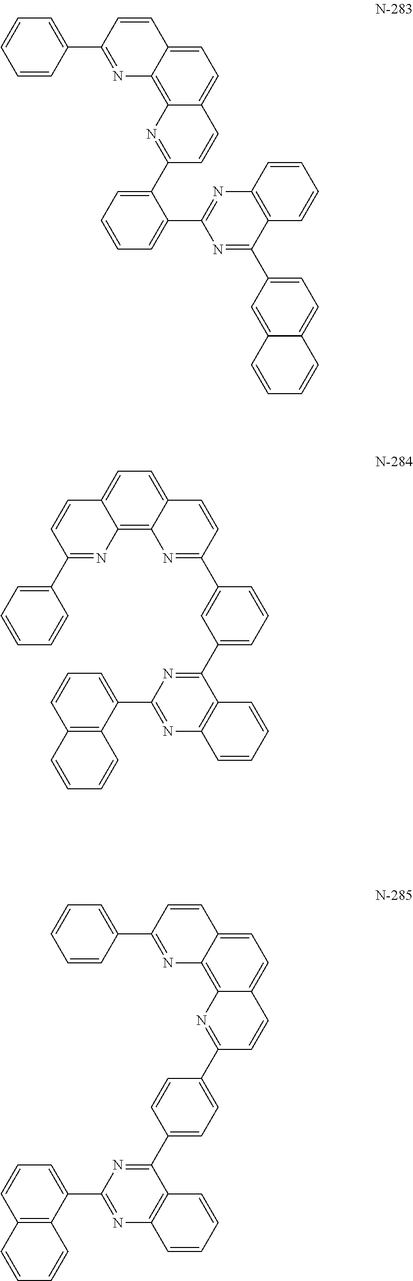 [SMILES: c1ccc(-c2ccc3ccc4ccc(-c5ccccc5-c5nc(-c6ccc7ccccc7c6)c6ccccc6n5)nc4c3n2)cc1 ; c1ccc(-c2ccc3ccc4ccc(-c5cccc(-c6nc(-c7cccc8ccccc78)nc7ccccc67)c5)nc4c3n2)cc1 ; c1ccc(-c2ccc3ccc4ccc(-c5ccc(-c6nc(-c7cccc8ccccc78)nc7ccccc67)cc5)nc4c3n2)cc1]

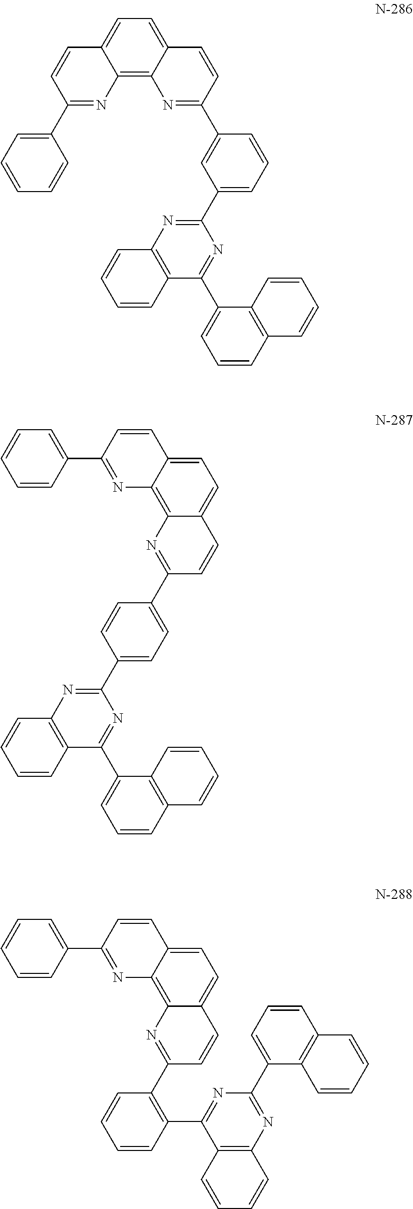 [SMILES: c1ccc(-c2ccc3ccc4ccc(-c5cccc(-c6nc(-c7cccc8ccccc78)c7ccccc7n6)c5)nc4c3n2)cc1 ; c1ccc(-c2ccc3ccc4ccc(-c5ccc(-c6nc(-c7cccc8ccccc78)c7ccccc7n6)cc5)nc4c3n2)cc1 ; c1ccc(-c2ccc3ccc4ccc(-c5ccccc5-c5nc(-c6cccc7ccccc67)nc6ccccc56)nc4c3n2)cc1]

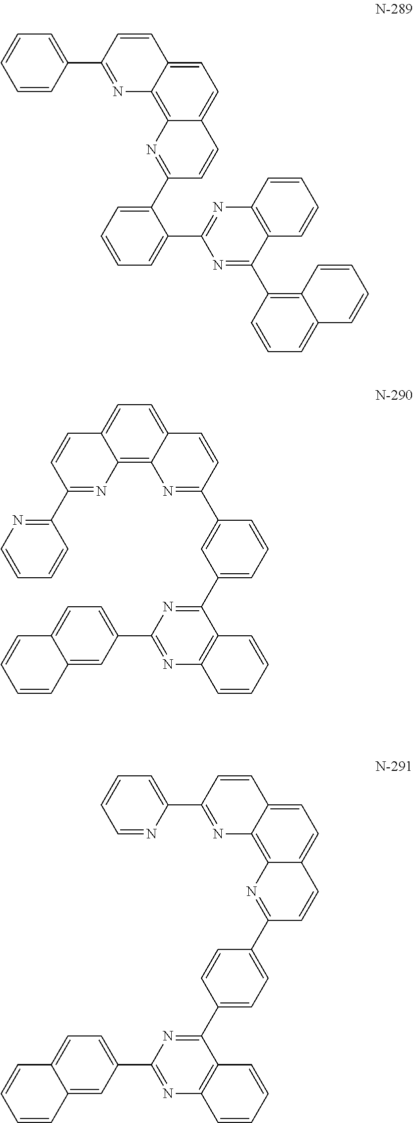 [SMILES: c1ccc(-c2ccc3ccc4ccc(-c5ccccc5-c5nc(-c6cccc7ccccc67)c6ccccc6n5)nc4c3n2)cc1 ; c1ccc(-c2ccc3ccc4ccc(-c5cccc(-c6nc(-c7ccc8ccccc8c7)nc7ccccc67)c5)nc4c3n2)nc1 ; c1ccc(-c2ccc3ccc4ccc(-c5ccc(-c6nc(-c7ccc8ccccc8c7)nc7ccccc67)cc5)nc4c3n2)nc1]

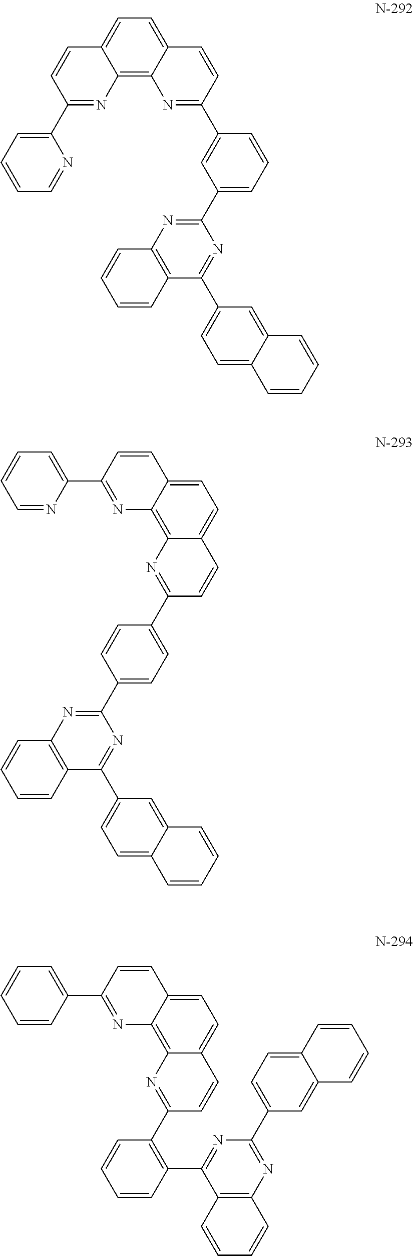 [SMILES: c1ccc(-c2ccc3ccc4ccc(-c5cccc(-c6nc(-c7ccc8ccccc8c7)c7ccccc7n6)c5)nc4c3n2)nc1 ; c1ccc(-c2ccc3ccc4ccc(-c5ccc(-c6nc(-c7ccc8ccccc8c7)c7ccccc7n6)cc5)nc4c3n2)nc1 ; c1ccc(-c2ccc3ccc4ccc(-c5ccccc5-c5nc(-c6ccc7ccccc7c6)nc6ccccc56)nc4c3n2)cc1]

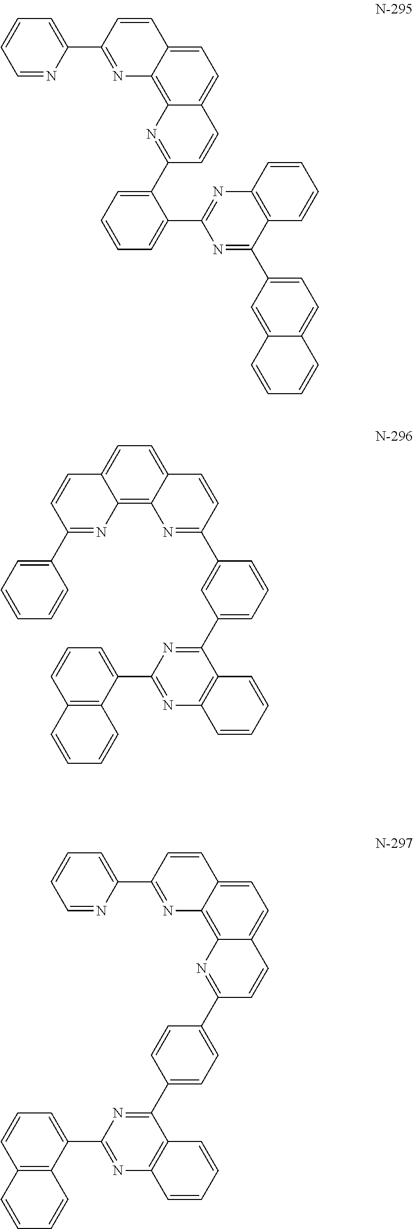 [SMILES: c1ccc(-c2ccc3ccc4ccc(-c5ccccc5-c5nc(-c6ccc7ccccc7c6)c6ccccc6n5)nc4c3n2)nc1 ; c1ccc(-c2ccc3ccc4ccc(-c5cccc(-c6nc(-c7cccc8ccccc78)nc7ccccc67)c5)nc4c3n2)cc1 ; c1ccc(-c2ccc3ccc4ccc(-c5ccc(-c6nc(-c7cccc8ccccc78)nc7ccccc67)cc5)nc4c3n2)nc1]

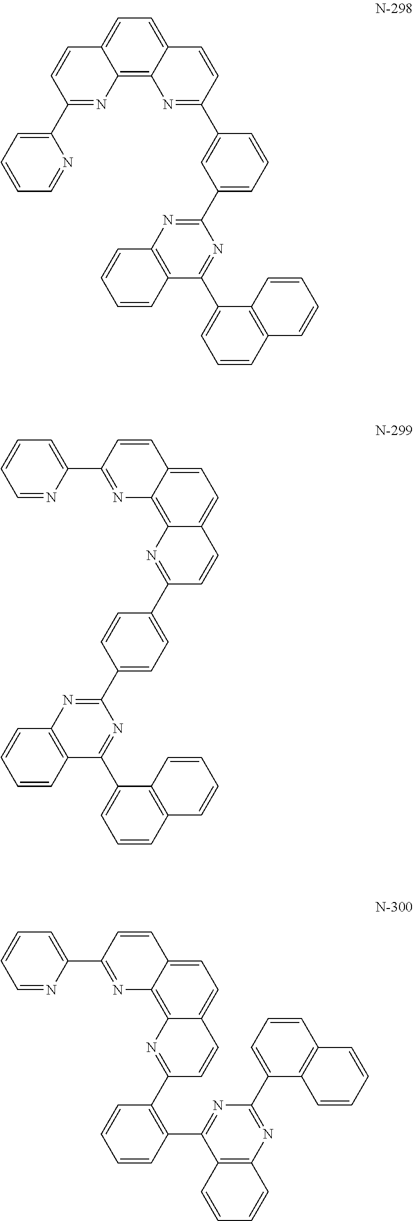 [SMILES: c1ccc(-c2ccc3ccc4ccc(-c5cccc(-c6nc(-c7cccc8ccccc78)c7ccccc7n6)c5)nc4c3n2)nc1 ; c1ccc(-c2ccc3ccc4ccc(-c5ccc(-c6nc(-c7cccc8ccccc78)c7ccccc7n6)cc5)nc4c3n2)nc1 ; c1ccc(-c2ccc3ccc4ccc(-c5ccccc5-c5nc(-c6cccc7ccccc67)nc6ccccc56)nc4c3n2)nc1]

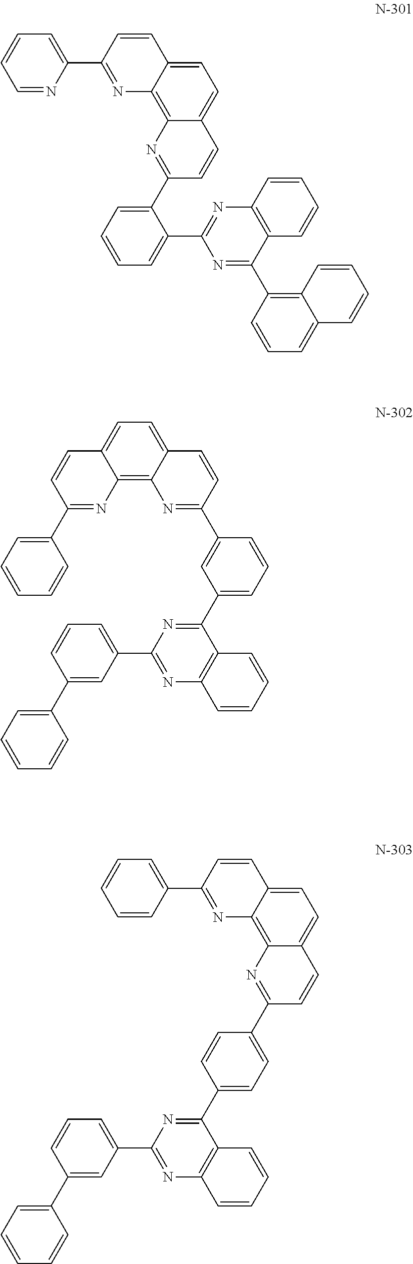 [SMILES: c1ccc(-c2ccc3ccc4ccc(-c5ccccc5-c5nc(-c6cccc7ccccc67)c6ccccc6n5)nc4c3n2)nc1 ; c1ccc(-c2cccc(-c3nc(-c4cccc(-c5ccc6ccc7ccc(-c8ccccc8)nc7c6n5)c4)c4ccccc4n3)c2)cc1 ; c1ccc(-c2cccc(-c3nc(-c4ccc(-c5ccc6ccc7ccc(-c8ccccc8)nc7c6n5)cc4)c4ccccc4n3)c2)cc1]

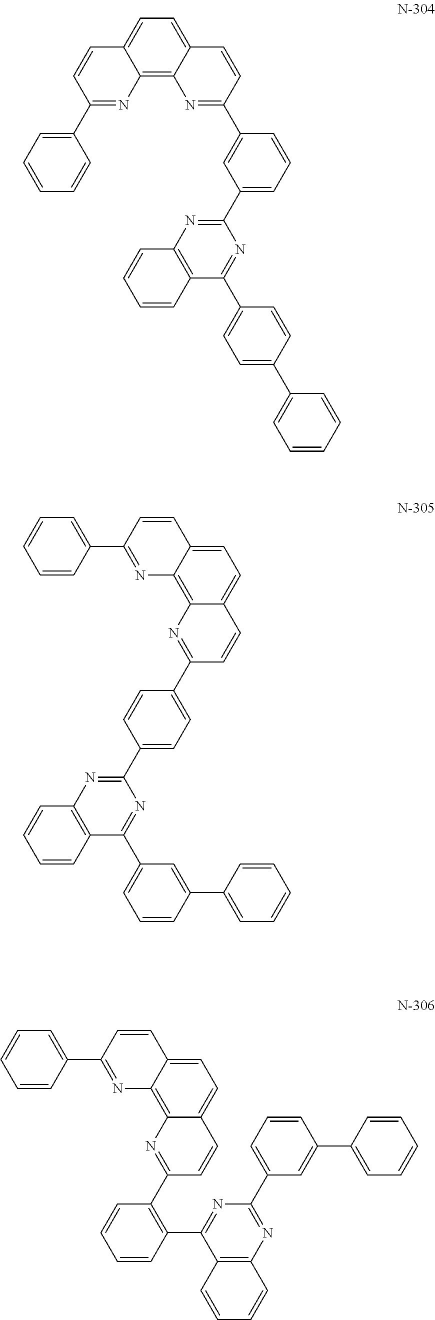 [SMILES: c1ccc(-c2ccc(-c3nc(-c4cccc(-c5ccc6ccc7ccc(-c8ccccc8)nc7c6n5)c4)nc4ccccc34)cc2)cc1 ; c1ccc(-c2cccc(-c3nc(-c4ccc(-c5ccc6ccc7ccc(-c8ccccc8)nc7c6n5)cc4)nc4ccccc34)c2)cc1 ; c1ccc(-c2cccc(-c3nc(-c4ccccc4-c4ccc5ccc6ccc(-c7ccccc7)nc6c5n4)c4ccccc4n3)c2)cc1]

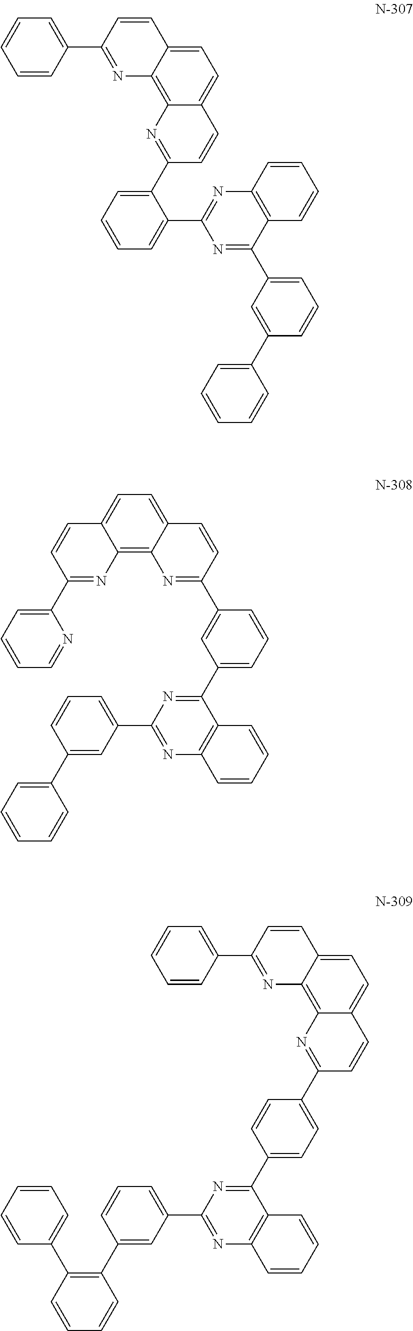 [SMILES: c1ccc(-c2cccc(-c3nc(-c4ccccc4-c4ccc5ccc6ccc(-c7ccccc7)nc6c5n4)nc4ccccc34)c2)cc1 ; c1ccc(-c2cccc(-c3nc(-c4cccc(-c5ccc6ccc7ccc(-c8ccccn8)nc7c6n5)c4)c4ccccc4n3)c2)cc1 ; c1ccc(-c2ccc3ccc4ccc(-c5ccc(-c6nc(-c7cccc(-c8ccccc8-c8ccccc8)c7)nc7ccccc67)cc5)nc4c3n2)cc1]

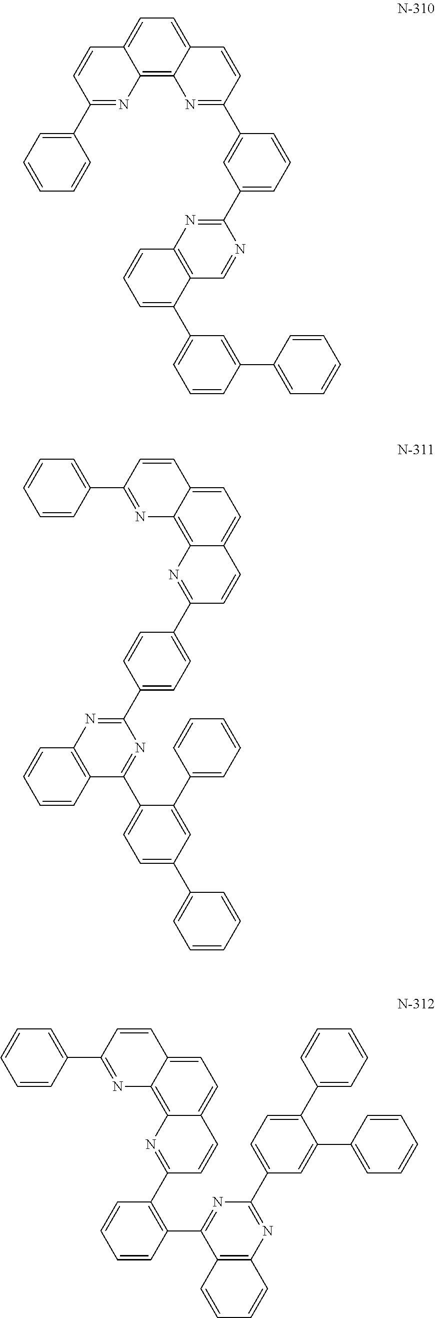 [SMILES: c1ccc(-c2cccc(-c3cccc4nc(-c5cccc(-c6ccc7ccc8ccc(-c9ccccc9)nc8c7n6)c5)ncc34)c2)cc1 ; c1ccc(-c2ccc(-c3nc(-c4ccc(-c5ccc6ccc7ccc(-c8ccccc8)nc7c6n5)cc4)nc4ccccc34)c(-c3ccccc3)c2)cc1 ; c1ccc(-c2ccc3ccc4ccc(-c5ccccc5-c5nc(-c6ccc(-c7ccccc7)c(-c7ccccc7)c6)nc6ccccc56)nc4c3n2)cc1]

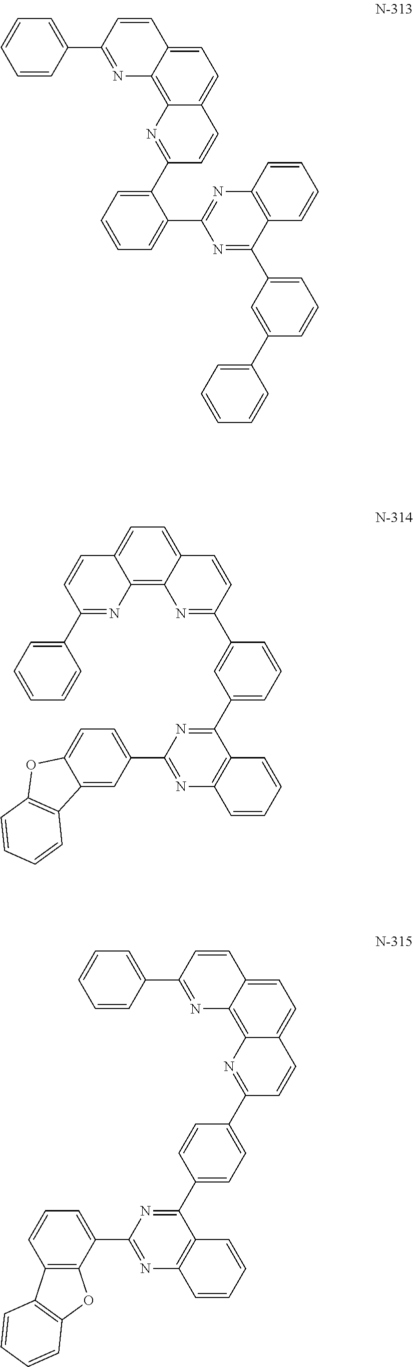 [SMILES: c1ccc(-c2cccc(-c3nc(-c4ccccc4-c4ccc5ccc6ccc(-c7ccccc7)nc6c5n4)nc4ccccc34)c2)cc1 ; c1ccc(-c2ccc3ccc4ccc(-c5cccc(-c6nc(-c7ccc8oc9ccccc9c8c7)nc7ccccc67)c5)nc4c3n2)cc1 ; c1ccc(-c2ccc3ccc4ccc(-c5ccc(-c6nc(-c7cccc8c7oc7ccccc78)nc7ccccc67)cc5)nc4c3n2)cc1]

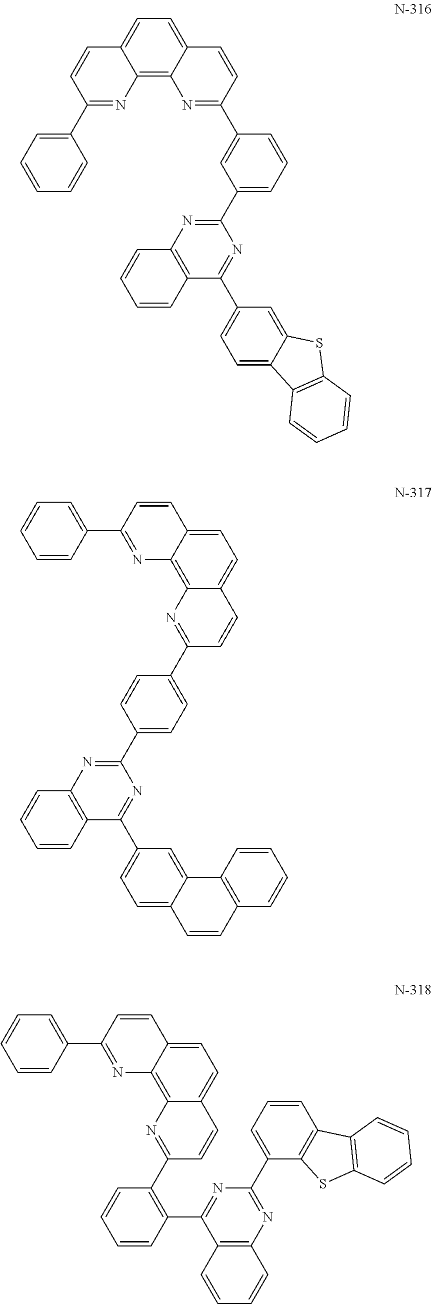 [SMILES: c1ccc(-c2ccc3ccc4ccc(-c5cccc(-c6nc(-c7ccc8c(c7)sc7ccccc78)c7ccccc7n6)c5)nc4c3n2)cc1 ; c1ccc(-c2ccc3ccc4ccc(-c5ccc(-c6nc(-c7ccc8ccc9ccccc9c8c7)c7ccccc7n6)cc5)nc4c3n2)cc1 ; c1ccc(-c2ccc3ccc4ccc(-c5ccccc5-c5nc(-c6cccc7c6sc6ccccc67)nc6ccccc56)nc4c3n2)cc1]

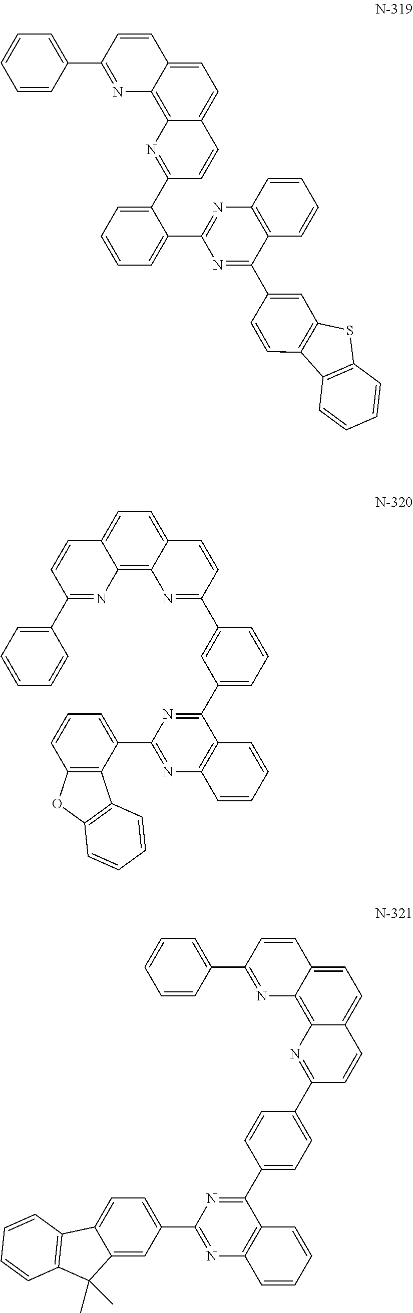 [SMILES: c1ccc(-c2ccc3ccc4ccc(-c5ccccc5-c5nc(-c6ccc7c(c6)sc6ccccc67)c6ccccc6n5)nc4c3n2)cc1 ; c1ccc(-c2ccc3ccc4ccc(-c5cccc(-c6nc(-c7cccc8oc9ccccc9c78)nc7ccccc67)c5)nc4c3n2)cc1 ; CC1(C)c2ccccc2-c2ccc(-c3nc(-c4ccc(-c5ccc6ccc7ccc(-c8ccccc8)nc7c6n5)cc4)c4ccccc4n3)cc21]

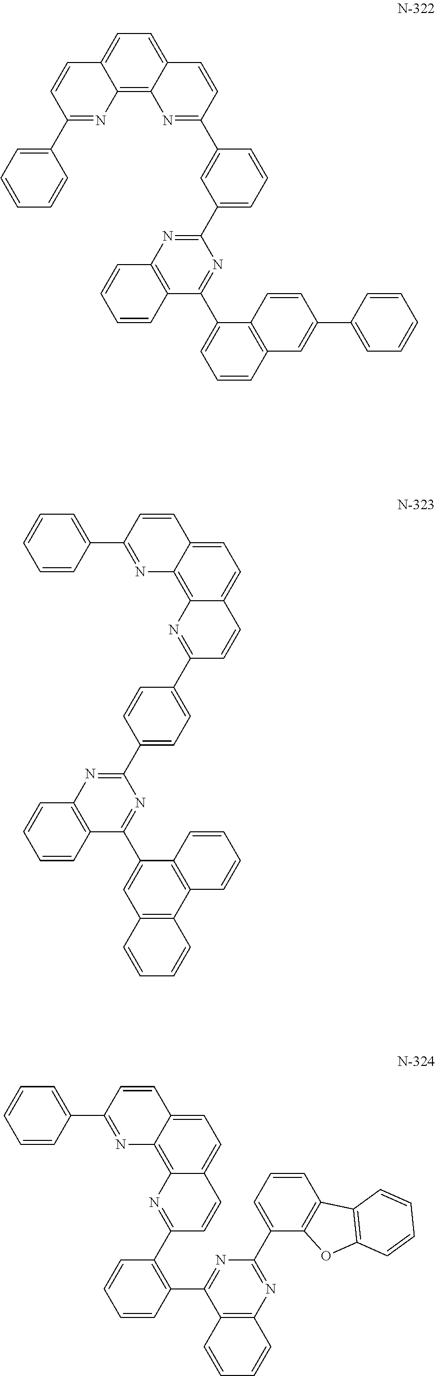 [SMILES: c1ccc(-c2ccc3c(-c4nc(-c5cccc(-c6ccc7ccc8ccc(-c9ccccc9)nc8c7n6)c5)nc5ccccc45)cccc3c2)cc1 ; c1ccc(-c2ccc3ccc4ccc(-c5ccc(-c6nc(-c7cc8ccccc8c8ccccc78)c7ccccc7n6)cc5)nc4c3n2)cc1 ; c1ccc(-c2ccc3ccc4ccc(-c5ccccc5-c5nc(-c6cccc7c6oc6ccccc67)nc6ccccc56)nc4c3n2)cc1]

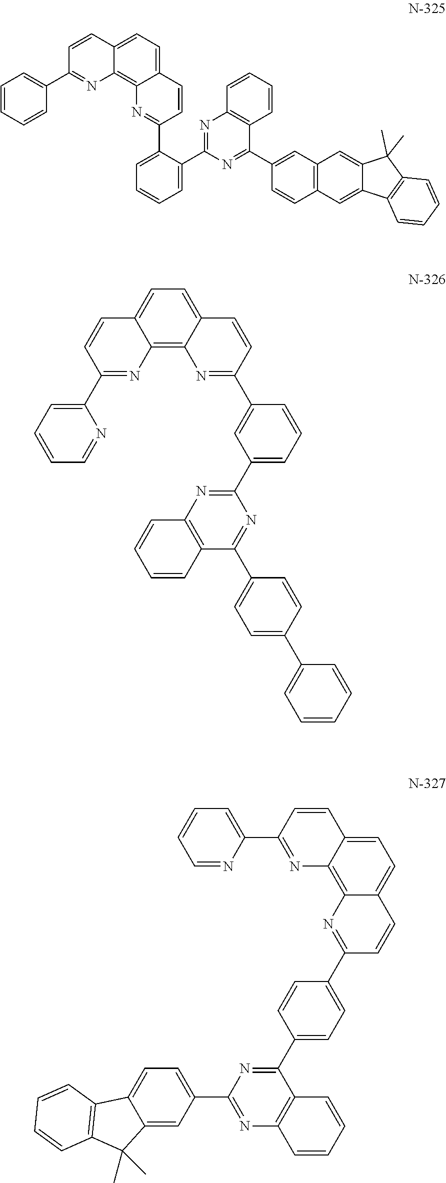 [SMILES: CC1(C)c2ccccc2-c2cc3ccc(-c4nc(-c5ccccc5-c5ccc6ccc7ccc(-c8ccccc8)nc7c6n5)nc5ccccc45)cc3cc21 ; c1ccc(-c2ccc(-c3nc(-c4cccc(-c5ccc6ccc7ccc(-c8ccccn8)nc7c6n5)c4)nc4ccccc34)cc2)cc1 ; CC1(C)c2ccccc2-c2ccc(-c3nc(-c4ccc(-c5ccc6ccc7ccc(-c8ccccn8)nc7c6n5)cc4)c4ccccc4n3)cc21]

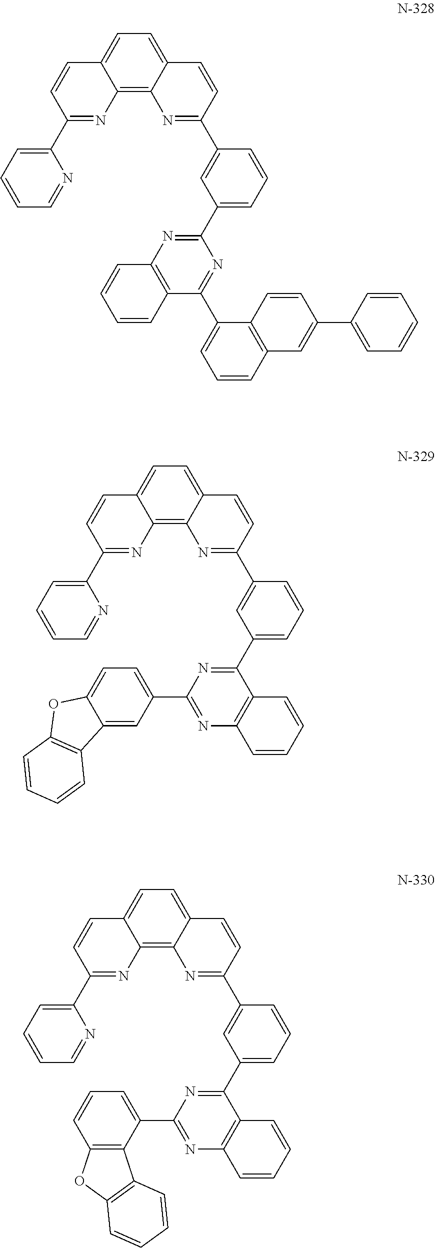 [SMILES: c1ccc(-c2ccc3c(-c4nc(-c5cccc(-c6ccc7ccc8ccc(-c9ccccn9)nc8c7n6)c5)nc5ccccc45)cccc3c2)cc1 ; c1ccc(-c2ccc3ccc4ccc(-c5cccc(-c6nc(-c7ccc8oc9ccccc9c8c7)nc7ccccc67)c5)nc4c3n2)nc1 ; c1ccc(-c2ccc3ccc4ccc(-c5cccc(-c6nc(-c7cccc8oc9ccccc9c78)nc7ccccc67)c5)nc4c3n2)nc1]

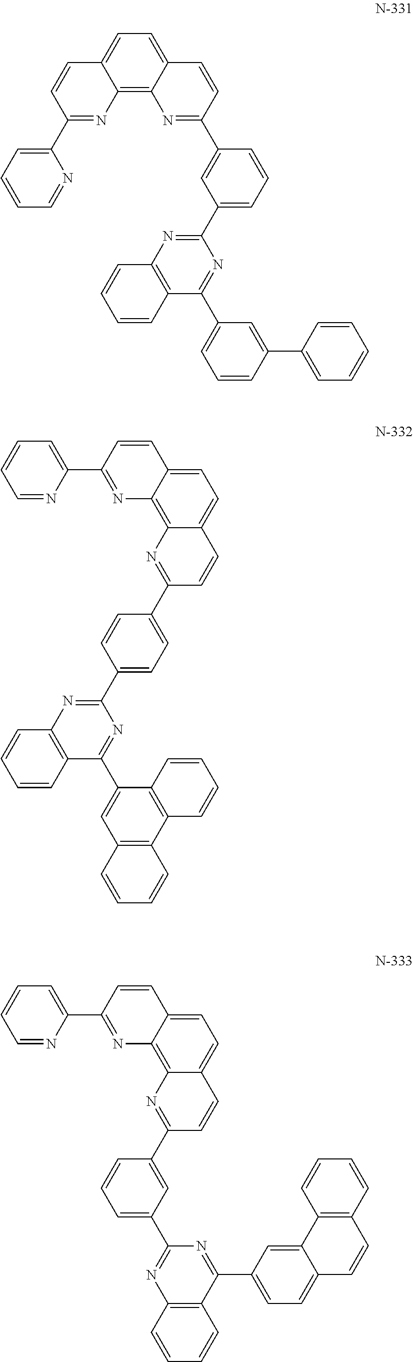 [SMILES: c1ccc(-c2cccc(-c3nc(-c4cccc(-c5ccc6ccc7ccc(-c8ccccn8)nc7c6n5)c4)nc4ccccc34)c2)cc1 ; c1ccc(-c2ccc3ccc4ccc(-c5ccc(-c6nc(-c7cc8ccccc8c8ccccc78)c7ccccc7n6)cc5)nc4c3n2)nc1 ; c1ccc(-c2ccc3ccc4ccc(-c5cccc(-c6nc(-c7ccc8ccc9ccccc9c8c7)c7ccccc7n6)c5)nc4c3n2)nc1]

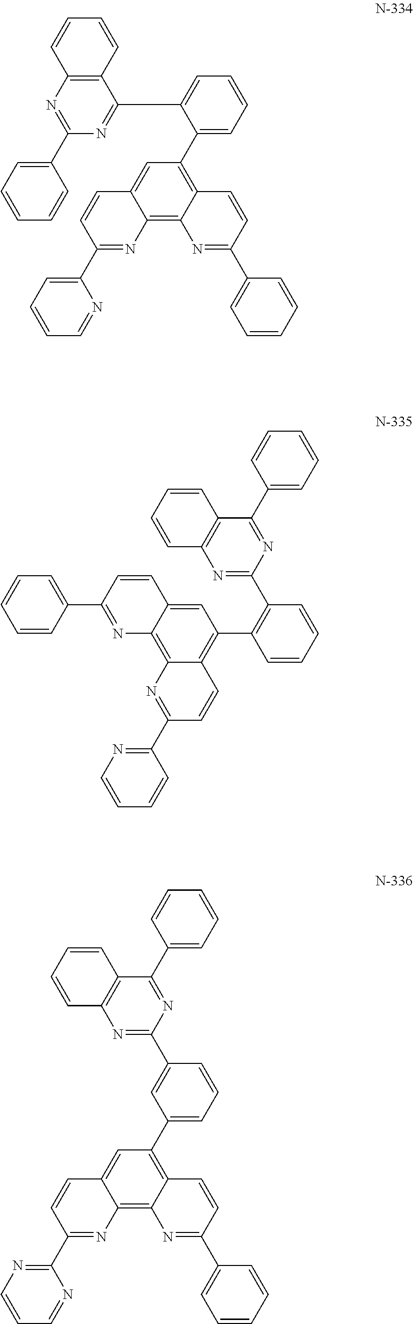 [SMILES: c1ccc(-c2ccc3c(-c4ccccc4-c4nc(-c5ccccc5)nc5ccccc45)cc4ccc(-c5ccccn5)nc4c3n2)cc1 ; c1ccc(-c2ccc3cc(-c4ccccc4-c4nc(-c5ccccc5)c5ccccc5n4)c4ccc(-c5ccccn5)nc4c3n2)cc1 ; c1ccc(-c2ccc3c(-c4cccc(-c5nc(-c6ccccc6)c6ccccc6n5)c4)cc4ccc(-c5ncccn5)nc4c3n2)cc1]

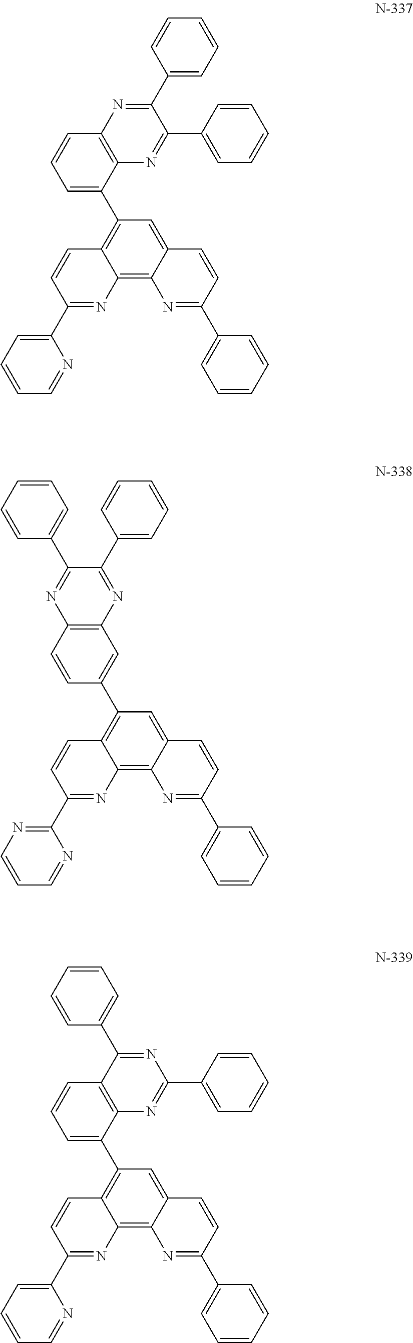 [SMILES: c1ccc(-c2ccc3cc(-c4cccc5nc(-c6ccccc6)c(-c6ccccc6)nc45)c4ccc(-c5ccccn5)nc4c3n2)cc1 ; c1ccc(-c2ccc3cc(-c4ccc5nc(-c6ccccc6)c(-c6ccccc6)nc5c4)c4ccc(-c5ncccn5)nc4c3n2)cc1 ; c1ccc(-c2ccc3cc(-c4cccc5c(-c6ccccc6)nc(-c6ccccc6)nc45)c4ccc(-c5ccccn5)nc4c3n2)cc1]

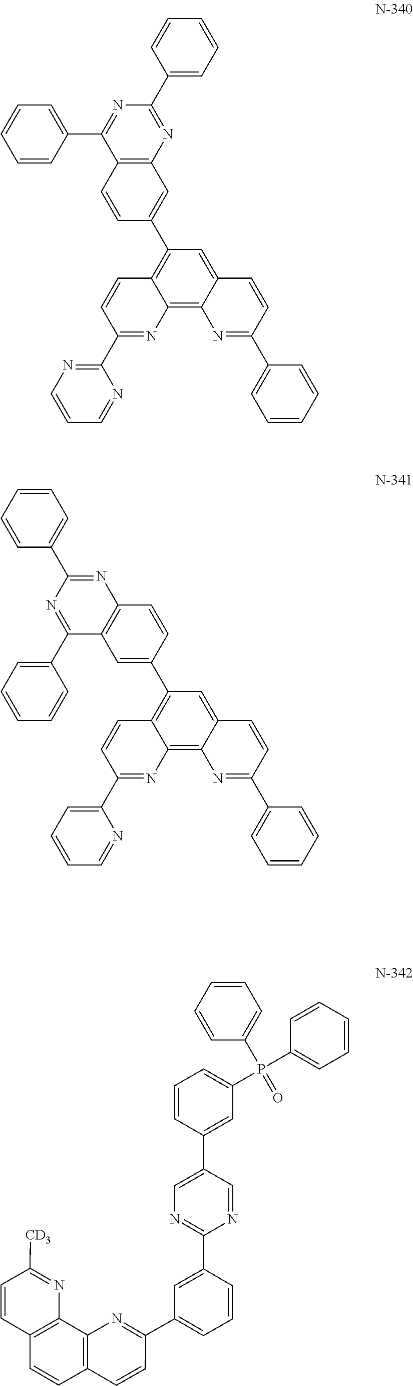 [SMILES: c1ccc(-c2ccc3cc(-c4ccc5c(-c6ccccc6)nc(-c6ccccc6)nc5c4)c4ccc(-c5ncccn5)nc4c3n2)cc1 ; c1ccc(-c2ccc3cc(-c4ccc5nc(-c6ccccc6)nc(-c6ccccc6)c5c4)c4ccc(-c5ccccn5)nc4c3n2)cc1 ; [2H]C([2H])([2H])c1ccc2ccc3ccc(-c4cccc(-c5ncc(-c6cccc(P(=O)(c7ccccc7)c7ccccc7)c6)cn5)c4)nc3c2n1]

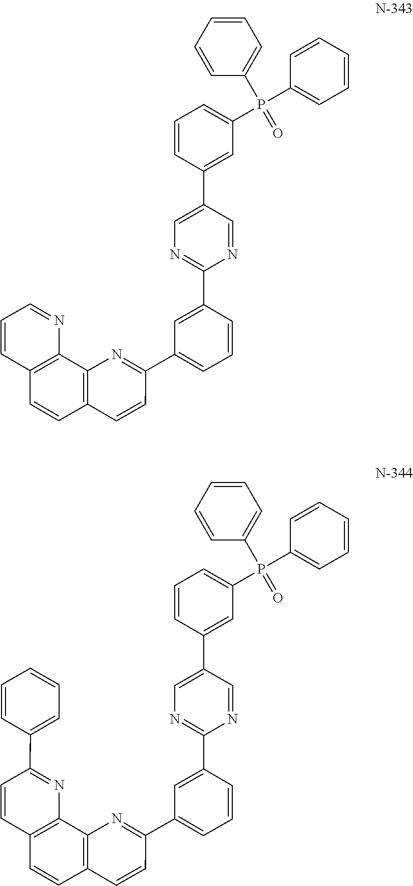 [SMILES: O=P(c1ccccc1)(c1ccccc1)c1cccc(-c2cnc(-c3cccc(-c4ccc5ccc6cccnc6c5n4)c3)nc2)c1 ; O=P(c1ccccc1)(c1ccccc1)c1cccc(-c2cnc(-c3cccc(-c4ccc5ccc6ccc(-c7ccccc7)nc6c5n4)c3)nc2)c1]

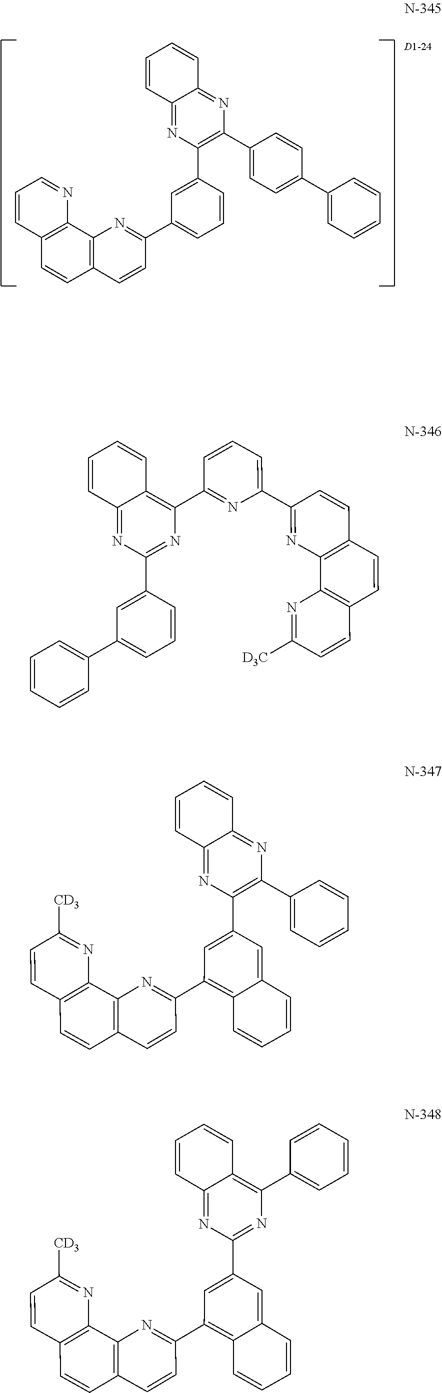

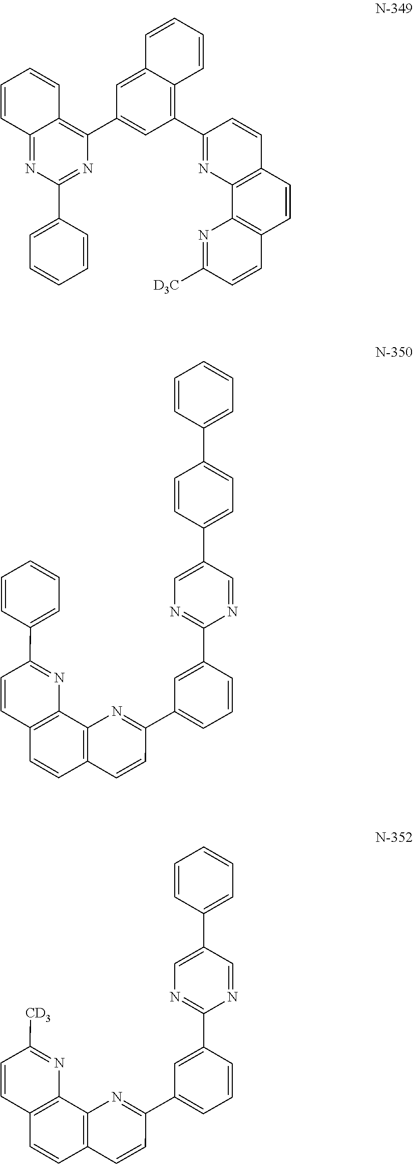 [SMILES: [2H]C([2H])([2H])c1ccc2ccc3ccc(-c4cc(-c5nc(-c6ccccc6)nc6ccccc56)cc5ccccc45)nc3c2n1 ; c1ccc(-c2ccc(-c3cnc(-c4cccc(-c5ccc6ccc7ccc(-c8ccccc8)nc7c6n5)c4)nc3)cc2)cc1 ; [2H]C([2H])([2H])c1ccc2ccc3ccc(-c4cccc(-c5ncc(-c6ccccc6)cn5)c4)nc3c2n1]

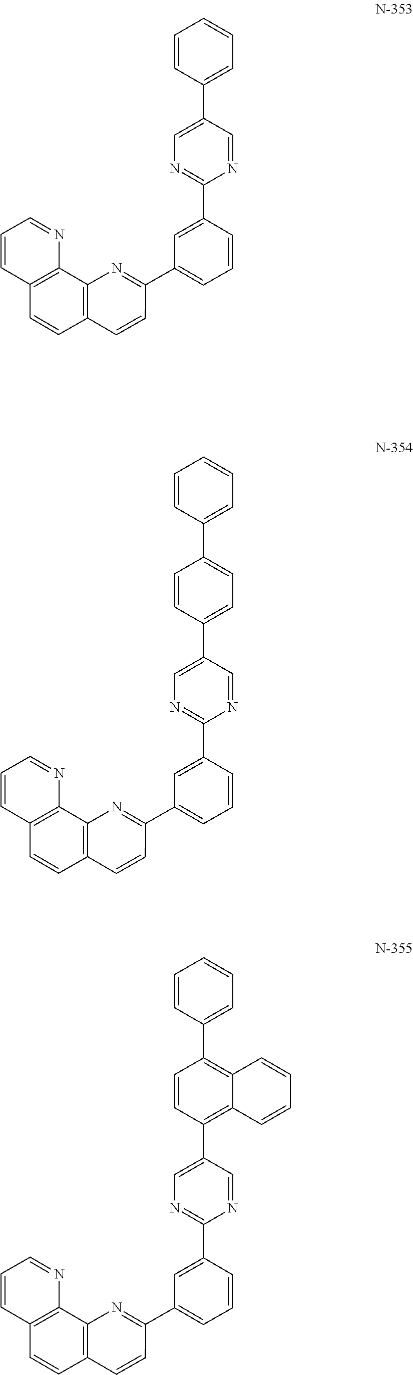 [SMILES: c1ccc(-c2cnc(-c3cccc(-c4ccc5ccc6cccnc6c5n4)c3)nc2)cc1 ; c1ccc(-c2ccc(-c3cnc(-c4cccc(-c5ccc6ccc7cccnc7c6n5)c4)nc3)cc2)cc1 ; c1ccc(-c2ccc(-c3cnc(-c4cccc(-c5ccc6ccc7cccnc7c6n5)c4)nc3)c3ccccc23)cc1]

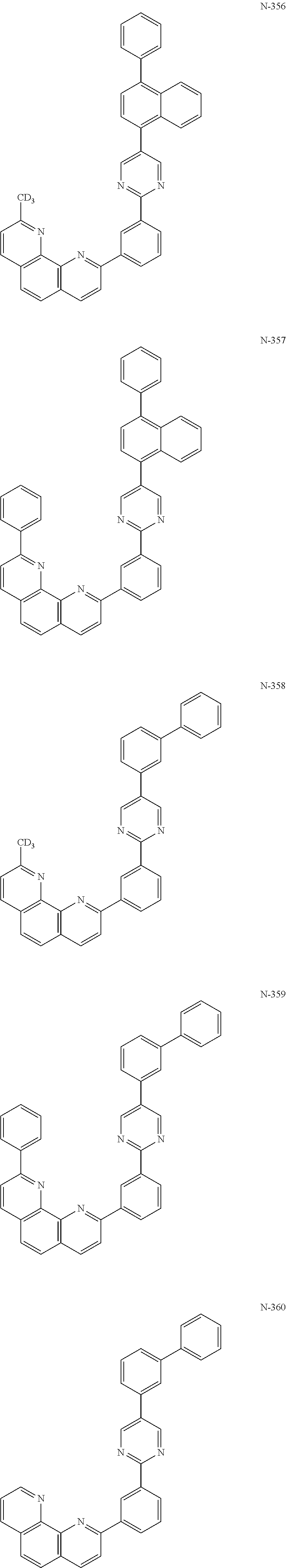 [SMILES: [2H]C([2H])([2H])c1ccc2ccc3ccc(-c4cccc(-c5ncc(-c6ccc(-c7ccccc7)c7ccccc67)cn5)c4)nc3c2n1 ; c1ccc(-c2ccc3ccc4ccc(-c5cccc(-c6ncc(-c7ccc(-c8ccccc8)c8ccccc78)cn6)c5)nc4c3n2)cc1 ; [2H]C([2H])([2H])c1ccc2ccc3ccc(-c4cccc(-c5ncc(-c6cccc(-c7ccccc7)c6)cn5)c4)nc3c2n1 ; c1ccc(-c2cccc(-c3cnc(-c4cccc(-c5ccc6ccc7ccc(-c8ccccc8)nc7c6n5)c4)nc3)c2)cc1 ; c1ccc(-c2cccc(-c3cnc(-c4cccc(-c5ccc6ccc7cccnc7c6n5)c4)nc3)c2)cc1]

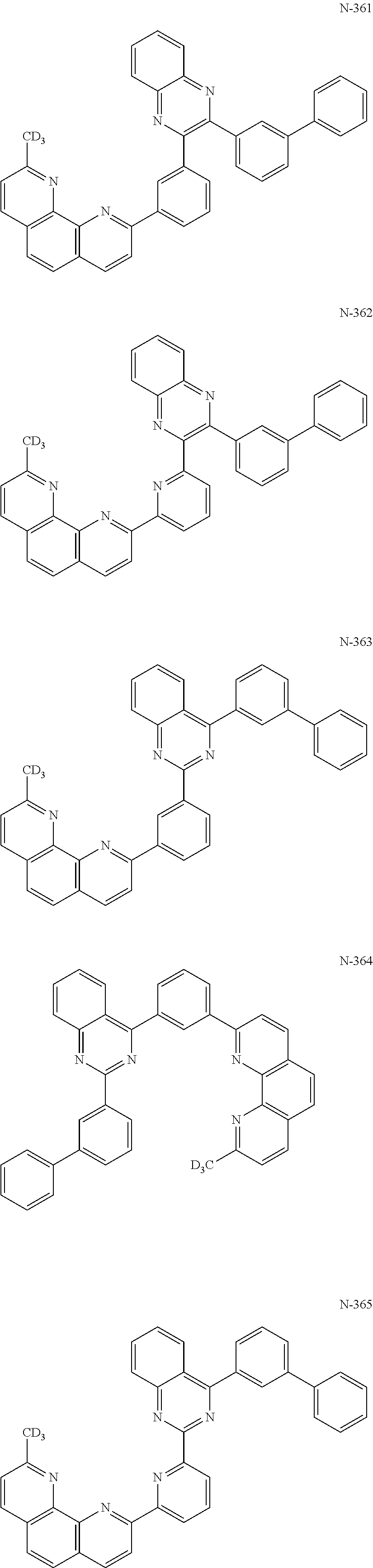 [SMILES: [2H]C([2H])([2H])c1ccc2ccc3ccc(-c4cccc(-c5nc6ccccc6nc5-c5cccc(-c6ccccc6)c5)c4)nc3c2n1 ; [2H]C([2H])([2H])c1ccc2ccc3ccc(-c4cccc(-c5nc6ccccc6nc5-c5cccc(-c6ccccc6)c5)n4)nc3c2n1 ; [2H]C([2H])([2H])c1ccc2ccc3ccc(-c4cccc(-c5nc(-c6cccc(-c7ccccc7)c6)c6ccccc6n5)c4)nc3c2n1 ; [2H]C([2H])([2H])c1ccc2ccc3ccc(-c4cccc(-c5nc(-c6cccc(-c7ccccc7)c6)nc6ccccc56)c4)nc3c2n1 ; [2H]C([2H])([2H])c1ccc2ccc3ccc(-c4cccc(-c5nc(-c6cccc(-c7ccccc7)c6)c6ccccc6n5)n4)nc3c2n1]

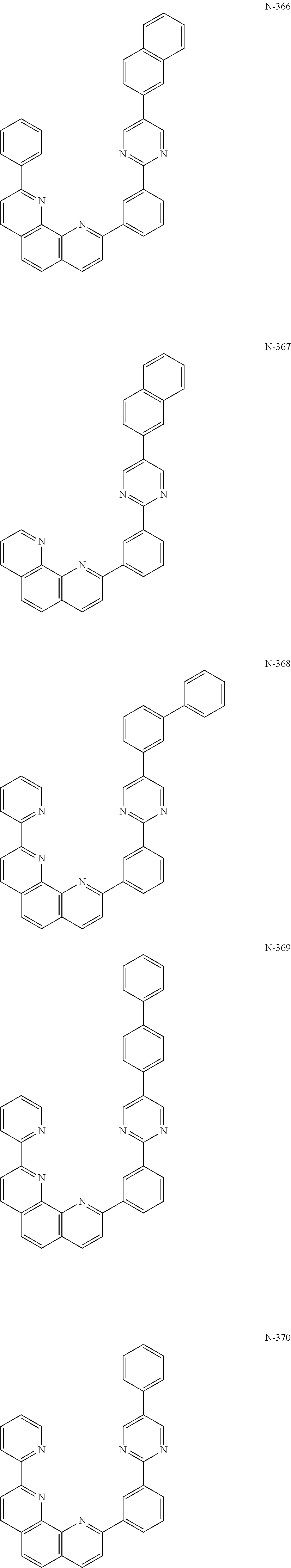 [SMILES: c1ccc(-c2ccc3ccc4ccc(-c5cccc(-c6ncc(-c7ccc8ccccc8c7)cn6)c5)nc4c3n2)cc1 ; c1cc(-c2ccc3ccc4cccnc4c3n2)cc(-c2ncc(-c3ccc4ccccc4c3)cn2)c1 ; c1ccc(-c2cccc(-c3cnc(-c4cccc(-c5ccc6ccc7ccc(-c8ccccn8)nc7c6n5)c4)nc3)c2)cc1 ; c1ccc(-c2ccc(-c3cnc(-c4cccc(-c5ccc6ccc7ccc(-c8ccccn8)nc7c6n5)c4)nc3)cc2)cc1 ; c1ccc(-c2cnc(-c3cccc(-c4ccc5ccc6ccc(-c7ccccn7)nc6c5n4)c3)nc2)cc1]

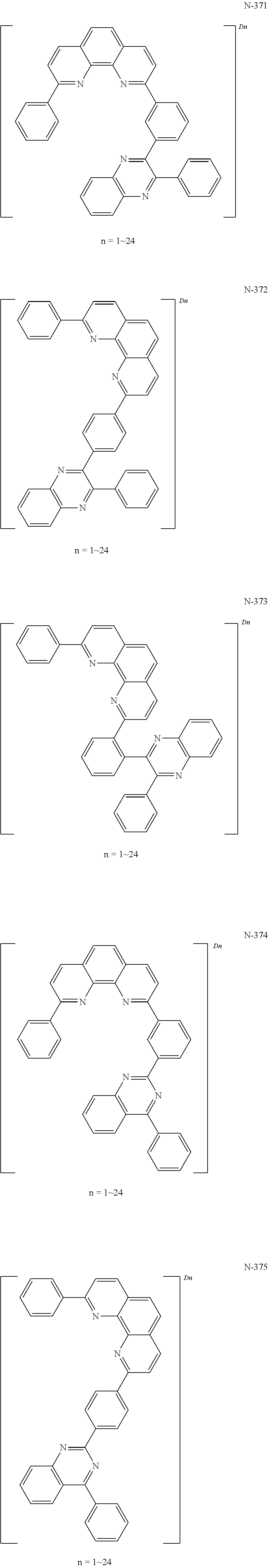

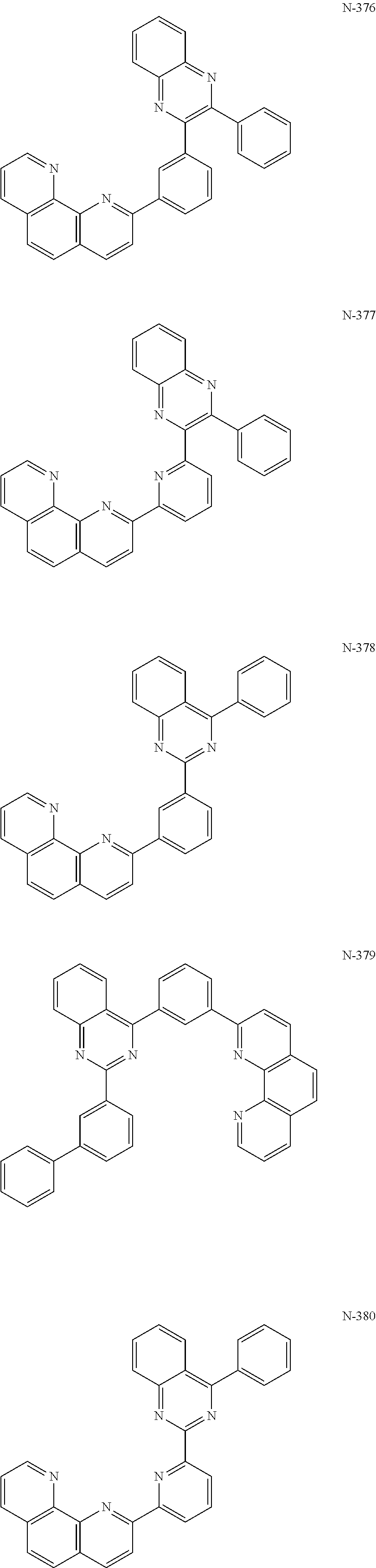 [SMILES: c1ccc(-c2nc3ccccc3nc2-c2cccc(-c3ccc4ccc5cccnc5c4n3)c2)cc1 ; c1ccc(-c2nc3ccccc3nc2-c2cccc(-c3ccc4ccc5cccnc5c4n3)n2)cc1 ; c1ccc(-c2nc(-c3cccc(-c4ccc5ccc6cccnc6c5n4)c3)nc3ccccc23)cc1 ; c1ccc(-c2cccc(-c3nc(-c4cccc(-c5ccc6ccc7cccnc7c6n5)c4)c4ccccc4n3)c2)cc1 ; c1ccc(-c2nc(-c3cccc(-c4ccc5ccc6cccnc6c5n4)n3)nc3ccccc23)cc1]

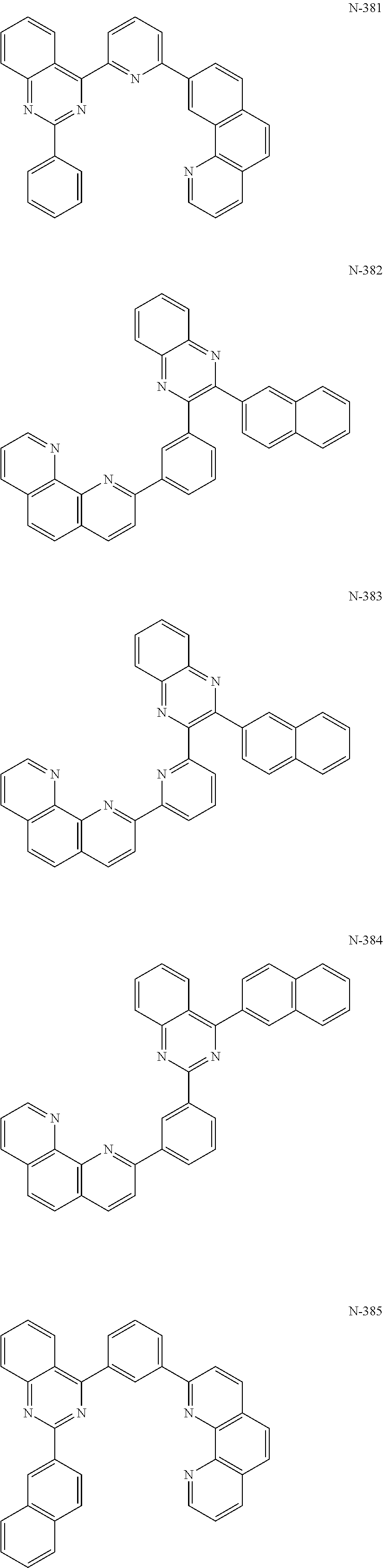 [SMILES: c1ccc(-c2nc(-c3cccc(-c4ccc5ccc6cccnc6c5c4)n3)c3ccccc3n2)cc1 ; c1cc(-c2ccc3ccc4cccnc4c3n2)cc(-c2nc3ccccc3nc2-c2ccc3ccccc3c2)c1 ; c1cc(-c2ccc3ccc4cccnc4c3n2)nc(-c2nc3ccccc3nc2-c2ccc3ccccc3c2)c1 ; c1cc(-c2ccc3ccc4cccnc4c3n2)cc(-c2nc(-c3ccc4ccccc4c3)c3ccccc3n2)c1 ; c1cc(-c2ccc3ccc4cccnc4c3n2)cc(-c2nc(-c3ccc4ccccc4c3)nc3ccccc23)c1]

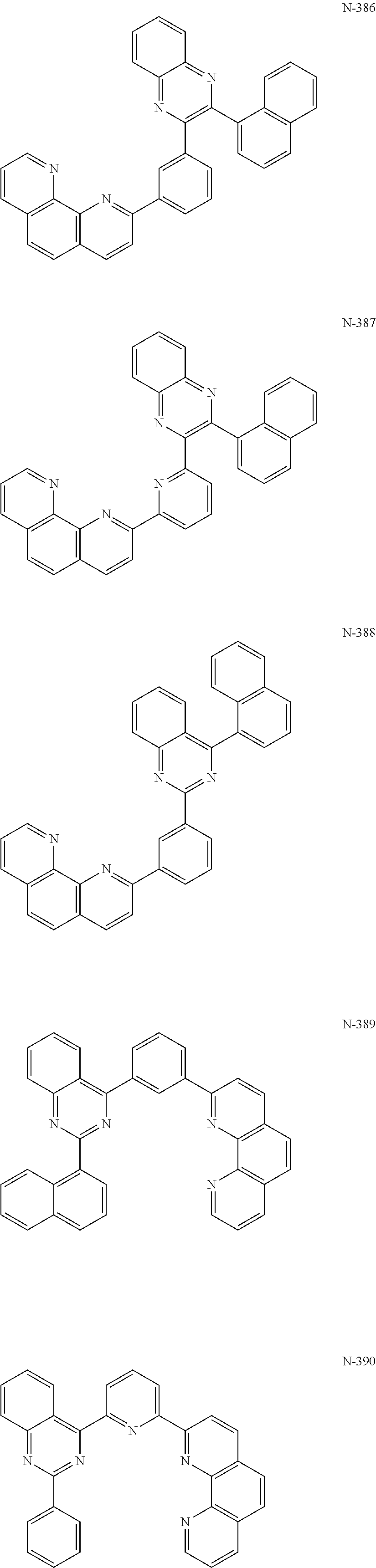 [SMILES: c1cc(-c2ccc3ccc4cccnc4c3n2)cc(-c2nc3ccccc3nc2-c2cccc3ccccc23)c1 ; c1cc(-c2ccc3ccc4cccnc4c3n2)nc(-c2nc3ccccc3nc2-c2cccc3ccccc23)c1 ; c1cc(-c2ccc3ccc4cccnc4c3n2)cc(-c2nc(-c3cccc4ccccc34)c3ccccc3n2)c1 ; c1cc(-c2ccc3ccc4cccnc4c3n2)cc(-c2nc(-c3cccc4ccccc34)nc3ccccc23)c1 ; c1ccc(-c2nc(-c3cccc(-c4ccc5ccc6cccnc6c5n4)n3)c3ccccc3n2)cc1]

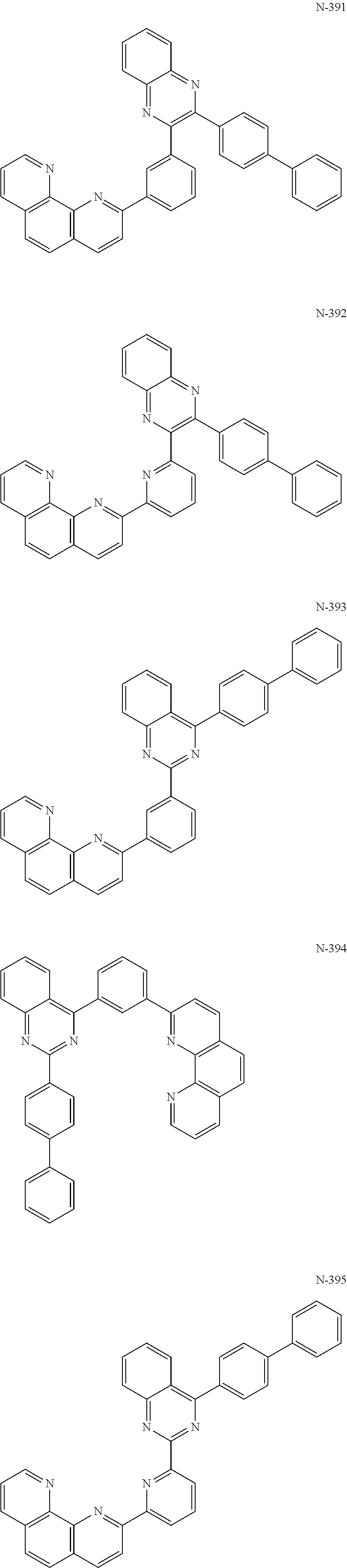 [SMILES: c1ccc(-c2ccc(-c3nc4ccccc4nc3-c3cccc(-c4ccc5ccc6cccnc6c5n4)c3)cc2)cc1 ; c1ccc(-c2ccc(-c3nc4ccccc4nc3-c3cccc(-c4ccc5ccc6cccnc6c5n4)n3)cc2)cc1 ; c1ccc(-c2ccc(-c3nc(-c4cccc(-c5ccc6ccc7cccnc7c6n5)c4)nc4ccccc34)cc2)cc1 ; c1ccc(-c2ccc(-c3nc(-c4cccc(-c5ccc6ccc7cccnc7c6n5)c4)c4ccccc4n3)cc2)cc1 ; c1ccc(-c2ccc(-c3nc(-c4cccc(-c5ccc6ccc7cccnc7c6n5)n4)nc4ccccc34)cc2)cc1]

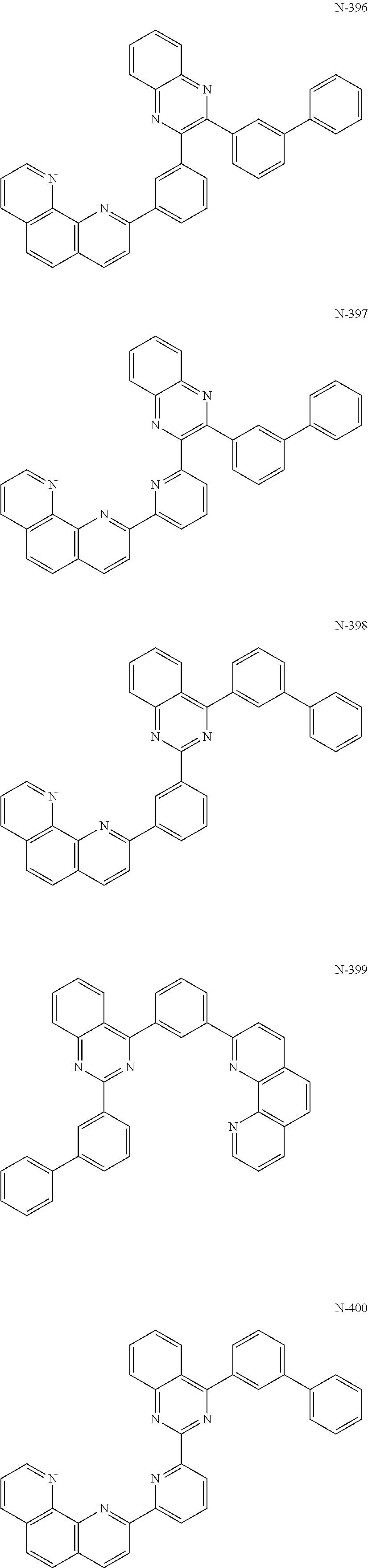 [SMILES: c1ccc(-c2cccc(-c3nc4ccccc4nc3-c3cccc(-c4ccc5ccc6cccnc6c5n4)c3)c2)cc1 ; c1ccc(-c2cccc(-c3nc4ccccc4nc3-c3cccc(-c4ccc5ccc6cccnc6c5n4)n3)c2)cc1 ; c1ccc(-c2cccc(-c3nc(-c4cccc(-c5ccc6ccc7cccnc7c6n5)c4)nc4ccccc34)c2)cc1 ; c1ccc(-c2cccc(-c3nc(-c4cccc(-c5ccc6ccc7cccnc7c6n5)c4)c4ccccc4n3)c2)cc1 ; c1ccc(-c2cccc(-c3nc(-c4cccc(-c5ccc6ccc7cccnc7c6n5)n4)nc4ccccc34)c2)cc1]

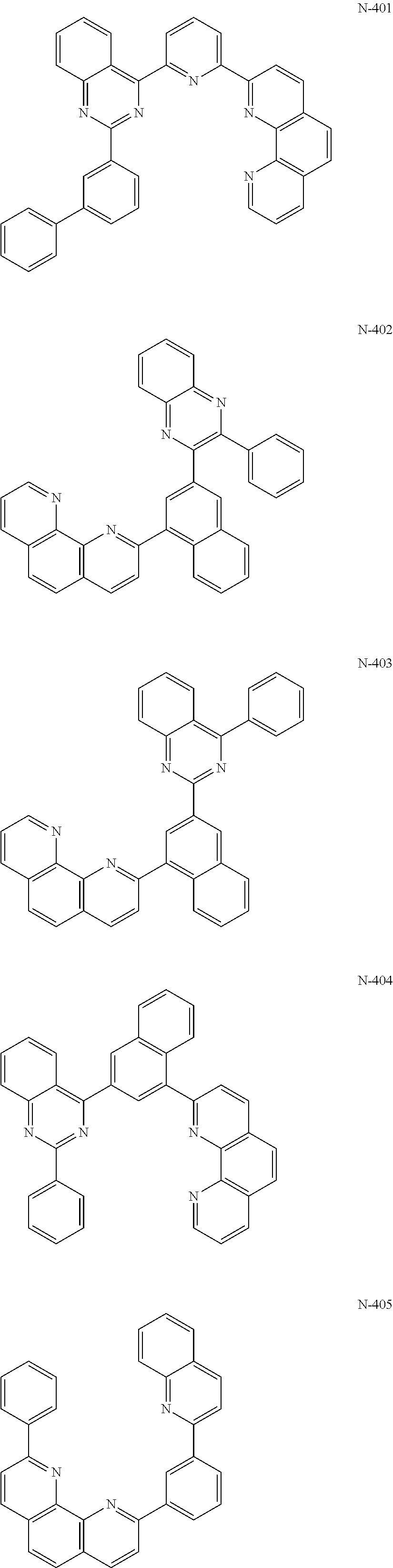 [SMILES: c1ccc(-c2cccc(-c3nc(-c4cccc(-c5ccc6ccc7cccnc7c6n5)n4)c4ccccc4n3)c2)cc1 ; c1ccc(-c2nc3ccccc3nc2-c2cc(-c3ccc4ccc5cccnc5c4n3)c3ccccc3c2)cc1 ; c1ccc(-c2nc(-c3cc(-c4ccc5ccc6cccnc6c5n4)c4ccccc4c3)nc3ccccc23)cc1 ; c1ccc(-c2nc(-c3cc(-c4ccc5ccc6cccnc6c5n4)c4ccccc4c3)c3ccccc3n2)cc1 ; c1ccc(-c2ccc3ccc4ccc(-c5cccc(-c6ccc7ccccc7n6)c5)nc4c3n2)cc1]

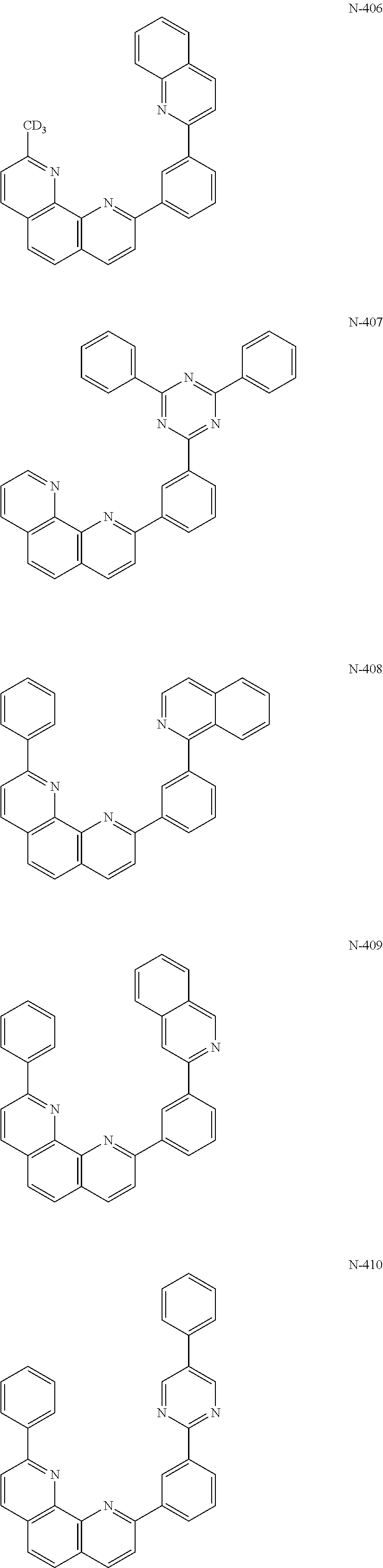 [SMILES: [2H]C([2H])([2H])c1ccc2ccc3ccc(-c4cccc(-c5ccc6ccccc6n5)c4)nc3c2n1 ; c1ccc(-c2nc(-c3ccccc3)nc(-c3cccc(-c4ccc5ccc6cccnc6c5n4)c3)n2)cc1 ; c1ccc(-c2ccc3ccc4ccc(-c5cccc(-c6nccc7ccccc67)c5)nc4c3n2)cc1 ; c1ccc(-c2ccc3ccc4ccc(-c5cccc(-c6cc7ccccc7cn6)c5)nc4c3n2)cc1 ; c1ccc(-c2cnc(-c3cccc(-c4ccc5ccc6ccc(-c7ccccc7)nc6c5n4)c3)nc2)cc1]

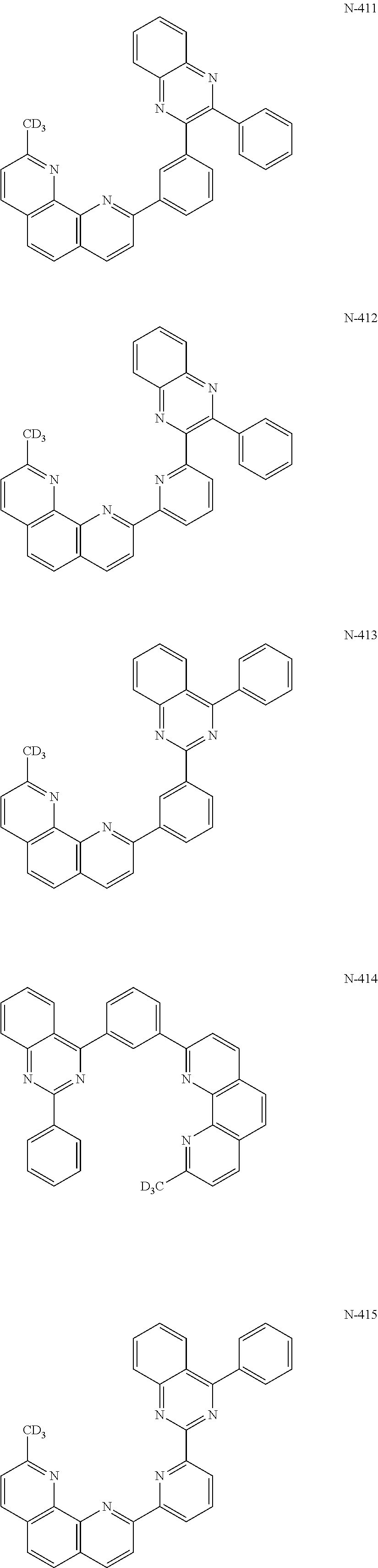 [SMILES: [2H]C([2H])([2H])c1ccc2ccc3ccc(-c4cccc(-c5nc6ccccc6nc5-c5ccccc5)c4)nc3c2n1 ; [2H]C([2H])([2H])c1ccc2ccc3ccc(-c4cccc(-c5nc6ccccc6nc5-c5ccccc5)n4)nc3c2n1 ; [2H]C([2H])([2H])c1ccc2ccc3ccc(-c4cccc(-c5nc(-c6ccccc6)c6ccccc6n5)c4)nc3c2n1 ; [2H]C([2H])([2H])c1ccc2ccc3ccc(-c4cccc(-c5nc(-c6ccccc6)nc6ccccc56)c4)nc3c2n1 ; [2H]C([2H])([2H])c1ccc2ccc3ccc(-c4cccc(-c5nc(-c6ccccc6)c6ccccc6n5)n4)nc3c2n1]

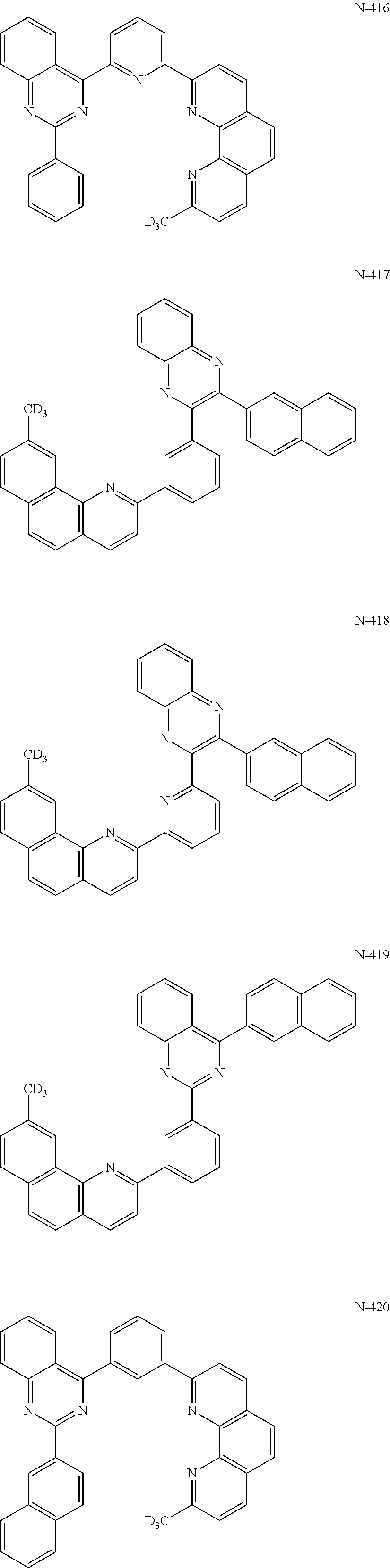 [SMILES: [2H]C([2H])([2H])c1ccc2ccc3ccc(-c4cccc(-c5nc(-c6ccccc6)nc6ccccc56)n4)nc3c2n1 ; [2H]C([2H])([2H])c1ccc2ccc3ccc(-c4cccc(-c5nc6ccccc6nc5-c5ccc6ccccc6c5)c4)nc3c2c1 ; [2H]C([2H])([2H])c1ccc2ccc3ccc(-c4cccc(-c5nc6ccccc6nc5-c5ccc6ccccc6c5)n4)nc3c2c1 ; [2H]C([2H])([2H])c1ccc2ccc3ccc(-c4cccc(-c5nc(-c6ccc7ccccc7c6)c6ccccc6n5)c4)nc3c2c1 ; [2H]C([2H])([2H])c1ccc2ccc3ccc(-c4cccc(-c5nc(-c6ccc7ccccc7c6)nc6ccccc56)c4)nc3c2n1]

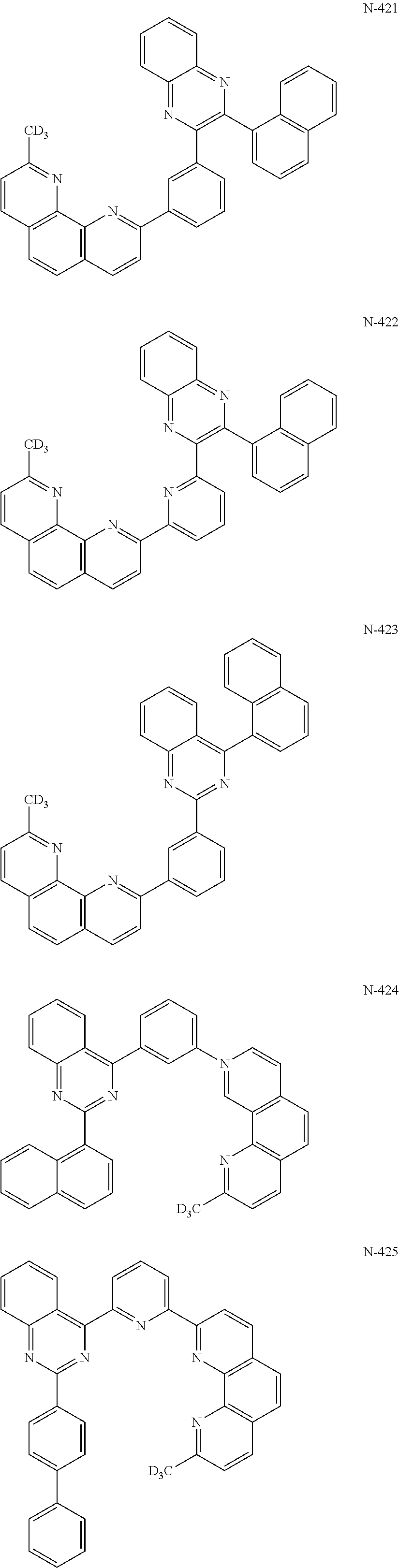

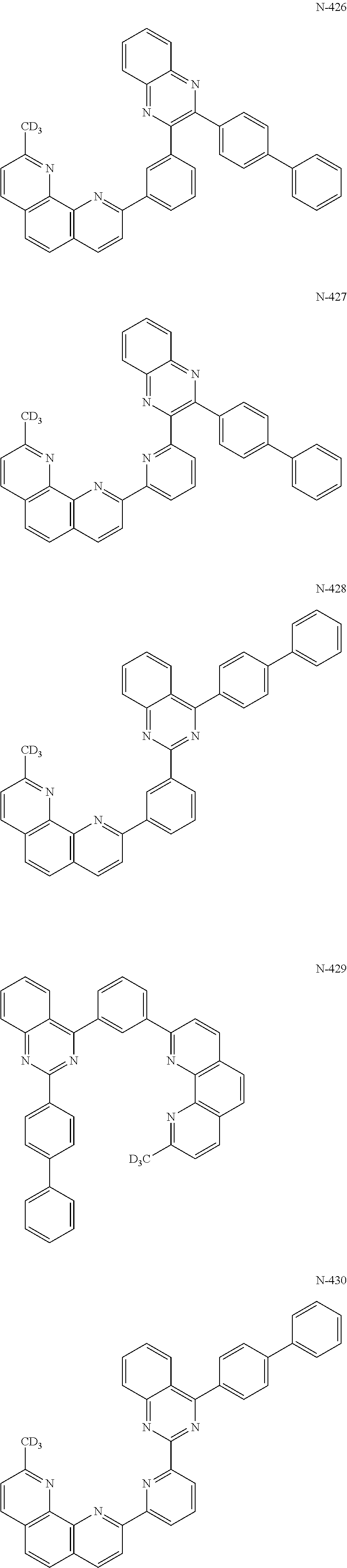 [SMILES: [2H]C([2H])([2H])c1ccc2ccc3ccc(-c4cccc(-c5nc6ccccc6nc5-c5ccc(-c6ccccc6)cc5)c4)nc3c2n1 ; [2H]C([2H])([2H])c1ccc2ccc3ccc(-c4cccc(-c5nc6ccccc6nc5-c5ccc(-c6ccccc6)cc5)n4)nc3c2n1 ; [2H]C([2H])([2H])c1ccc2ccc3ccc(-c4cccc(-c5nc(-c6ccc(-c7ccccc7)cc6)c6ccccc6n5)c4)nc3c2n1 ; [2H]C([2H])([2H])c1ccc2ccc3ccc(-c4cccc(-c5nc(-c6ccc(-c7ccccc7)cc6)nc6ccccc56)c4)nc3c2n1 ; [2H]C([2H])([2H])c1ccc2ccc3ccc(-c4cccc(-c5nc(-c6ccc(-c7ccccc7)cc6)c6ccccc6n5)n4)nc3c2n1]

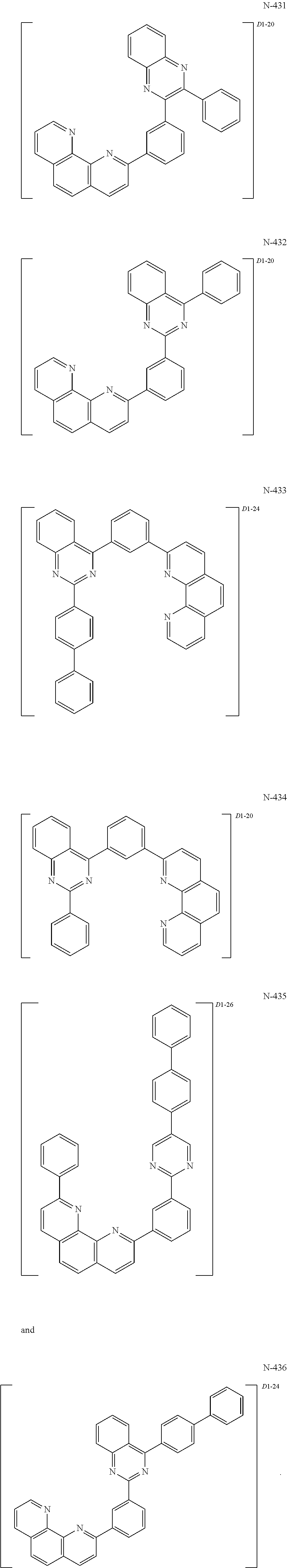

15. An organic electroluminescent device comprising the organic electroluminescent compound of claim 12.
